(19) United States
(12) Patent Application Publication
(10) Pub. No.: US 2018/0148429 A1
(43) Pub. Date: May 31, 2018

(54) SUBSTITUTED QUINOXALINE DERIVATIVES

(71) Applicant: SELVITA S.A., Kraków (PL)

(72) Inventors: Charles-Henry Robert Yves Fabritius, Kraków (PL); Mateusz Oktawian Nowak, Kraków (PL); Katarzyna Anna Wiklik, Kraków (PL); Aleksandra Barbara Sabiniarz, Kraków (PL); Marcin Dominik Bien, Suloszowa (PL); Anna Malgorzata Buda, Kraków (PL); Pawel Szczepan Guzik, Kraków (PL); Krzysztof Roman Jakubiec, Katowice (PL); Monika Maciuszek, Kraków (PL); Katarzyna Kwiecinska, Wroblew (PL); Mateusz Michal Tomczyk, Zabrze (PL); Michal Mikolaj Galezowski, Kraków (PL); Andrzej Gondela, Tychy (PL); Lukasz Piotr Dudek, Kraków (PL)

(21) Appl. No.: 15/572,470

(22) PCT Filed: May 12, 2016
(86) PCT No.: PCT/EP2016/000783

§ 371 Date: Nov. 7, 2017

(30) Foreign Application Priority Data
May 13, 2015 (EP) 15001451.2
May 13, 2015 (EP) 15460017.5

(51) Int. Cl. C07D 401/14; A61P 35/00; A61P 35/02; C07D 403/10; C07D 405/14; C07D 491/052; C07D 403/14; C07D 417/14; C07D 413/14; C07D 471/04; C07D 405/10; C07D 241/42; C07D 403/12; C07D 409/14

(52) U.S. Cl. CPC C07D 401/14; A61P 35/00; A61P 35/02; C07D 403/10; C07D 405/14; C07D 491/052; C07D 409/14; C07D 417/14; C07D 413/14; C07D 471/04; C07D 405/10; C07D 241/42; C07D 403/12; C07D 403/14

(57) ABSTRACT

The present invention relates to substituted quinoxaline derivatives. These compounds are useful for the prevention and/or treatment of several medical conditions including hyperproliferative disorders and diseases.

### FIELD OF THE INVENTION

The present invention relates to substituted quinoxaline derivatives. These compounds are useful for inhibiting 6-phosphofructo-2-kinase/fructose-2,6-bisphosphatase (PFKFB) and for the prevention and/or treatment of medical conditions affected by PFKFB activity. They are in particular useful for the prevention and/or treatment of cancer diseases.

### BACKGROUND OF THE INVENTION

Glycolysis is a non-oxidative metabolic pathway in which glucose is degraded by cells to generate ATP (adenosine triphosphate), i.e. energy. While normal, i.e. healthy cells are usually favoring this pathway for generating ATP only under anaerobic conditions, many cancer cells generate ATP—even in the presence of oxygen—from glucose via glycolysis; the glycolytic rate can be up to 200 times greater in malignant rapidly-growing tumor cells than in healthy cells. This switch of energy metabolism in cancer cells to the process of “aerobic glycolysis” is known as the “Warburg Effect” (D. G. Brooke et al., Biorganic & Medicinal Chemistry 22 (2014) 1029-1039; T. V. Pyrkov et al., ChemMedChem 2013, 8, 1322-1329).

The rate of glycolysis is regulated by several enzymes, including phosphofructokinase, that catalyze irreversible reactions in the course of glycolysis. 6-phosphofructo-1-kinase (PFK-1), the precursor of anaerobic ATP production, which converts fructose-6-phosphate (F6P) to fructose-1,6-bisphosphate (F1,6-BP), is considered to be the rate-limiting enzyme in the process of converting glucose into pyruvate. PFK-1 is allosterically activated by fructose-2,6-bisphosphate (F2,6-BP) which is synthesized from F6P by phosphofructokinase-2 (PFK-2; 6-phosphofructo-2-kinase/fructose-2,6-bisphosphatase, PFKFB). Four isoforms of the PFK-2 family are known, namely PFKFB1, PFKFB2, PFKFB3, and PFKFB4 (D. G. Brooke et al., Biorganic & Medicinal Chemistry 22 (2014) 1029-1039; T. V. Pyrkov et al., ChemMedChem 2013, 8, 1322-1329).

Many different cancer types exhibit an overexpression of PFK-2, particularly its isozymes PFKFB4 and hypoxia-inducible form PFKFB3. PFKFB3 is overexpressed in many cancer types including colon, prostate, pancreatic, breast, thyroid, leukemia, lung, ovarian tumors (D. G. Brooke et al., Biorganic & Medicinal Chemistry 22 (2014) 1029-1039; T. V. Pyrkov et al., ChemMedChem 2013, 8, 1322-1329). Overexpression of PFKFB4 has been associated, inter alia, with glioma, hepatic, bladder, and prostate cancer (T. V. Pyrkov et al., ChemMedChem 2013, 8, 1322-1329). Thus, 6-phosphofructo-2-kinase/fructose-2,6-bisphosphatase and in particular isoforms PFKFB3 and PFKFB4 are promising targets for cancer therapy by utilizing small molecules as inhibitors of these enzymes.

### DESCRIPTION OF THE INVENTION

It is an object of the present invention to provide inhibitors of PFKFB3 and/or PFKFB4 wherein that inhibitors may be useful for the prevention and/or treatment of medical conditions, disorders and/or diseases that are affected by PFKFB3 and/or PFKFB4 activity. It is a particular object of the present invention to provide compounds and such inhibitors for the treatment of hyperproliferative disorders, in particular cancer diseases.

The object has surprisingly been solved by compounds of formula (I)

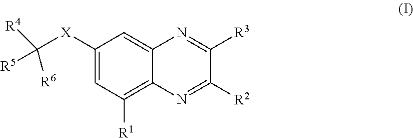

wherein
X denotes N—R⁷ or O;
R¹ denotes Ar^{X}, Ar^{X}—Ar^{Y}, Ar^{X}-Hetar^{Y}, Ar^{X}-Hetcyc^{Y}, Ar^{X}-LA^{Z}-Ar^{Y}, Ar^{X}-LA^{Z}-Hetar^{Y}, Ar^{X}-LA^{Z}-Hetcyc^{Y}, Hetar^{X}, Hetar^{X}-Ar^{Y}, Hetar^{X}-Hetar^{Y}, Hetar^{X}-Hetcyc^{Y}, Hetar^{X}-LA^{Z}-Ar^{Y}, Hetar^{X}-LA^{Z}-Hetar^{Y}, Hetar^{X}-LA^{Z}-Hetcyc^{Y}, Hetcyc^{X}, Hetcyc^{X}-Ar^{Y}, Hetcyc^{X}-Hetar^{Y}, Hetcyc^{X}-Hetcyc^{Y}, Hetcyc^{X}-LA^{Z}-Ar^{Y}, Hetcyc^{X}-LA^{Z}-Hetar^{Y}, Hetcyc^{X}-LA^{Z}-Hetcyc^{Y}, CA^{X};
R² and R³ denote independently from each other H, OH, SH, unsubstituted straight-chain or branched —C₁₋₆-alkyl, straight-chain or branched —C₂₋₆-alkenyl, unsubstituted straight-chain or branched —O—C₁₋₆-alkyl, straight-chain or branched —S—C₁₋₆-alkyl, Hal, —CN, —C(═O)—NH₂, —C(═O)—NH(C₁₋₄-alkyl), —C(═O)—N(C₁₋₄-alkyl)₂, —NH₂, —NH(C₁₋₄-alkyl), —N(C₁₋₄-alkyl)₂ which C₁₋₄-alkyl substituents may be the same or different and may be straight-chain or branched;
R⁴ denotes Ar^{X}, Ar^{X}—Ar^{Y}, Ar^{X}-Hetar^{Y}, Ar^{X}-Hetcyc^{Y}, Ar^{X}-LA^{Z}-Ar^{Y}, Ar^{X}-LA^{Z}-Hetar^{Y}, Ar^{X}-LA^{Z}-Hetcyc^{Y}, Hetar^{X}, Hetar^{X}-Ar^{Y}, Hetar^{X}-Hetar^{Y}, Hetar^{X}-Hetcyc^{Y}, Hetar^{X}-LA^{Z}-Ar^{Y}, Hetar^{X}-LA^{Z}-Hetar^{Y}, Hetar^{X}-LA^{Z}-Hetcyc^{Y}, Hetcyc^{X}, Hetcyc^{X}-Ar^{Y}, Hetcyc^{X}-Hetar^{Y}, Hetcyc^{X}-Hetcyc^{Y}, Hetcyc^{X}-LA^{Z}-Ar^{Y}, Hetcyc^{X}-LA^{Z}-Hetar^{Y}, Hetcyc^{X}-LA^{Z}-Hetcyc^{Y}, LA^{X}, LA^{Z}-Ar^{Y}, LA^{Z}-Hetar^{Y}, LA^{Z}-Hetcyc^{Y}, CA^{X};
R⁵ denotes H, Ar^{X}, Ar^{X}—Ar^{Y}, Ar^{X}-Hetar^{Y}, Ar^{X}-Hetcyc^{Y}, Ar^{X}-LA^{Z}-Ar^{Y}, Ar^{X}-LA^{Z}-Hetar^{Y}, Ar^{X}-LA^{Z}-Hetcyc^{Y}, Hetar^{X}, Hetar^{X}-Ar^{Y}, Hetar^{X}-Hetar^{Y}, Hetar^{X}-Hetcyc^{Y}, Hetar^{X}-LA^{Z}-Ar^{Y}, Hetar^{X}-LA^{Z}-Hetar^{Y}, Hetar^{X}-LA^{Z}-Hetcyc^{Y}, Hetcyc^{X}, Hetcyc^{X}-Ar^{Y}, Hetcyc^{X}-Hetar^{Y}, Hetcyc^{X}-Hetcyc^{Y}, Hetcyc^{X}-LA^{Z}-Ar^{Y}, Hetcyc^{X}-LA^{Z}-Hetar^{Y}, Hetcyc^{X}-LA^{Z}-Hetcyc^{Y}, LA^{X}, LA^{Z}-Ar^{Y}, LA^{Z}-Hetar^{Y}, LA^{Z}-Hetcyc^{Y}, CA^{X}, —CN, —NO₂, —SO₂NH₂, —SO₂NHR^{X7}, —SO₂NR^{X7}R^{X8}, —NH—SO₂—R^{X9}, —NR^{X7}—SO₂—R^{X9}, —SO₂—R^{X9}, —NH₂, —NHR^{X7}, —NR^{X7}R^{X8}, —OH, —O—R^{X9}, —CHO, —C(═O)—R^{X9}, —NH—(C₁₋₃-alkylene)-C(═O)—NH₂, —NH—(C₁₋₃-alkylene)-C(═O)—NHR^{X7}, —NH—(C₁₋₃-alkylene)-C(═O)—NR^{X7}R^{X8}, —NH—C(═O)—R^{X9}, —NR^{X7}—C(═O)—R^{X9}, —C(═O)—NH₂, —C(═O)—NHR^{X7}, —C(═O)—NR^{X7}R^{X8}, —C(═O)OH, —C(═O)OR^{X9};
or
R⁴ and R⁵ form together with the carbon atom to which they are attached to a saturated or partially unsaturated ring system A which ring system A is mono- or bicyclic and has 3, 4, 5, 6, 7, 8, 9, 10, 11 ring atoms and may contain no hetero ring atom or 1, 2, 3 hetero ring atom(s) independently from each other selected from N, O and/or S that ring system A may be unsubstituted or mono-, di- or trisubstituted with independently from each other R^{A1}, R^{A2}, R^{A3};
R⁶ denotes H, Ar^{X}, Ar^{X}—Ar^{Y}, Ar^{X}-Hetar^{Y}, Ar^{X}-Hetcyc^{Y}, Ar^{X}-LA^{Z}-Ar^{Y}, Ar^{X}-LA^{Z}-Hetar^{Y}, Ar^{X}-LA^{Z}-Hetcyc^{Y}, Hetar^{X}, Hetar^{X}-Ar^{Y}, Hetar^{X}-Hetar^{Y}, Hetar^{X}-Hetcyc^{Y}, Hetar^{X}-LA^{Z}-Ar^{Y}, Hetar^{X}-LA^{Z}-Hetar^{Y}, Hetar^{X}-LA^{Z}-Hetcyc^{Y}, Hetcyc^{X}, Hetcyc^{X}-Ar^{Y}, Hetcyc^{X}-Hetar^{Y}, Hetcyc^{X}-Hetcyc^{Y}, Hetcyc^{X}-LA^{Z}-Ar^{Y}, Hetcyc^{X}-LA^{Z}-Hetar^{Y}, Hetcyc^{X}-LA^{Z}-Hetcyc^{Y}, LA^{X}, LA^{Z}-Ar^{Y}, LA^{Z}-Hetar^{Y}, LA^{Z}-Hetcyc^{Y}, CA^{X};
or
R⁵ and R⁶ form together with the carbon atom to which they are attached to a saturated or partially unsaturated ring system D which ring system D is mono- or bicyclic and has 3, 4, 5, 6, 7, 8, 9, 10, 11 ring atoms and may contain no hetero ring atom or 1, 2, 3 hetero ring atom(s) independently from each other selected from N, O and/or S that ring system D may be unsubstituted or mono-, di- or trisubstituted with independently from each other R^{D1}, R^{D2}, R^{D3};
or
R⁵ and R⁶ form together with the carbon atom to which they are attached to a C═CHR^{D4} moiety;
R⁷ denotes H, Hetar^{X}, Hetcyc^{X}, LA^{X}, CA^{X};
Ar^{X} denotes a mono-, bi- or tricyclic aromatic ring system with 5, 6, 7, 8, 9, 10, 11, 12, 13, 14 ring carbon atoms which ring system may be unsubstituted or mono-, di- or trisubstituted with independently from each other R^{X1}, R^{X2}, R^{X3};
Ar^{Y} denotes a mono-, bi- or tricyclic aromatic ring system with 5, 6, 7, 8, 9, 10, 11, 12, 13, 14 ring carbon atoms which ring system may be unsubstituted or mono-, di- or trisubstituted with independently from each other R^{Y1}, R^{Y2}, R^{Y3};
Hetar^{X} denotes a mono, bi- or tricyclic aromatic ring system with 5, 6, 7, 8, 9, 10, 11, 12, 13, 14 ring atoms wherein 1, 2, 3, 4, 5 of said ring atoms is/are a hetero atom(s) selected from N, O and/or S and the remaining are carbon atoms, wherein that aromatic ring system may be unsubstituted or mono-, di- or trisubstituted with independently from each other R^{X1}, R^{X2}, R^{X3};
Hetar^{Y} denotes a mono, bi- or tricyclic aromatic ring system with 5, 6, 7, 8, 9, 10, 11, 12, 13, 14 ring atoms wherein 1, 2, 3, 4, 5 of said ring atoms is/are a hetero atom(s) selected from N, O and/or S and the remaining are carbon atoms, wherein that aromatic ring system may be unsubstituted or mono-, di- or trisubstituted with independently from each other R^{Y1}, R^{Y2}, R^{Y3};
Hetcyc^{X} denotes a saturated or partially unsaturated mono-, bi- or tricyclic heterocycle with 3, 4, 5, 6, 7, 8, 9, 10, 11, 12, 13, 14 ring atoms wherein 1, 2, 3, 4, 5 ring atom(s) is/are heteroatom(s) selected from N, O and/or S and the remaining ring atoms are carbon atoms, wherein that heterocycle may be unsubstituted or mono-, di- or trisubstituted with R^{X4}, R^{X5}, R^{X6};
Hetcyc^{Y} denotes a saturated or partially unsaturated mono-, bi- or tricyclic heterocycle with 3, 4, 5, 6, 7, 8, 9, 10, 11, 12, 13, 14 ring atoms wherein 1, 2, 3, 4, 5 ring atom(s) is/are heteroatom(s) selected from N, O and/or S and the remaining ring atoms are carbon atoms, wherein that heterocycle may be unsubstituted or mono-, di- or trisubstituted with R^{Y4}, R^{Y5}, R^{Y6};
R^{X1}, R^{X2}, R^{X3} denote independently from each other other H, Hal, LA^{X}, CA^{X}, —CN, —NO₂, —SO₂NH₂, —SO₂NHR^{X7}, —SO₂NR^{X7}R^{X8}, —NH—SO₂—R^{X9}, —NR^{X7}—SO₂—R^{X9}, —S—R^{X9}, S(═O)—R^{X9}, —SO₂—R^{X9}, —NH₂, —NHR^{X7}, —NR^{X7}R^{X8}, —OH, —O—R^{X9}, —CHO, —C(═O)—R^{X9}, —COOH, —C(═O)—O—R^{X9}, —C(═O)—NH₂, —C(═O)—NHR^{X7}, —C(═O)—NR^{X7}R^{X8}, —NH—(C₁₋₃-alkylene)-C(═O)—NH₂, —NH—(C₁₋₃-alkylene)-C(═O)—NHR^{X7}, —NH—(C₁₋₃-alkylene)-C(═O)—NR^{X7}R^{X8}, —NH—C(═O)—R^{X9}, —NR^{X7}—C(═O)—R^{X9}
or
two of R^{X1}, R^{X2}, R^{X3} form a divalent alkylene chain with 3, 4, 5 chain carbon atoms wherein 1 or 2 of non-adjacent CH₂ groups of the divalent alkylene chain may be replaced independently from each other by —N(H)—, —N(C₁₋₆-alkyl)-, —N(—C(═O)—C₁₋₄-alkyl)-, —O— —wherein that C₁₋₆-alkyl and C₁₋₄-alkyl radicals may be straight-chain or branched—and wherein 2 adjacent CH₂ groups may together be replaced by a —CH═CH— moiety, which divalent alkylene chain may be unsubstituted or mono- or disubstituted with independently from each other straight-chain or branched —C₁₋₆-alkyl or ═O (oxo);
R^{X4}, R^{X5}, R^{X6} denote independently from each other H, Hal, LA^{X}, CA^{X}, —CN, —NO₂, —SO₂NH₂, —SO₂NHR^{X7}, —SO₂NR^{X7}R^{X8}, —NH—SO₂—R^{X9}, —NR^{X7}—SO₂—R^{X9}, —S—R^{X9}, —S(═O)—R^{X9}, —SO₂—R^{X9}, —NH₂, —NHR^{X7}, —NR^{X7}R^{X8}, —OH, —O—R^{X9}, —CHO, —C(═O)—R^{X9}, —COOH, —C(═O)—O—R^{X9}, —C(═O)—NH₂, —C(═O)—NHR^{X7}, —C(═O)—NR^{X7}R^{X8}, —NH—(C₁₋₃-alkylene)-C(═O)—NH₂, —NH—(C₁₋₃-alkylene)-C(═O)—NHR^{X7}, —NH—(C₁₋₃-alkylene)-C(═O)—NR^{X7}R^{X8}, —NH—C(═O)—R^{X9}, —NR^{X7}—C(═O)—R^{X9}, oxo (═O);
R^{Y1}, R^{Y2}, R^{Y3} denote independently from each other H, Hal, LA^{Y}, CA^{Y}, —CN, —NO₂, —SO₂NH₂, —SO₂NHR^{Y7}, —SO₂NR^{Y7}R^{Y8}, —NH—SO₂—R^{Y9}, —NR^{Y7}—SO₂—R^{Y9}, —S—R^{Y9}, —S(═O)—R^{Y9}, —SO₂—R^{Y9}, —NH₂, —NHR^{Y7}, —NR^{Y7}R^{Y8}, —OH, —O—R^{Y9}, —CHO, —C(═O)—R^{Y9}, —COOH, —C(═O)—O—R^{Y9}, —C(═O)—NH₂, —C(═O)—NHR^{Y7}, —C(═O)—NR^{Y7}R^{Y8}, —NH—(C₁₋₃-alkylene)-C(═O)—NH₂, —NH—(C₁₋₃-alkylene)-C(═O)—NHR^{Y7}, —NH—(C₁₋₃-alkylene)-C(═O)—NR^{Y7}R^{Y8}, —NH—C(═O)—R^{Y9}, —NR^{Y7}—C(═O)—R^{Y9}
or
two of R^{Y1}, R^{Y2}, R^{Y3} form a divalent alkylene chain with 3, 4, 5 chain carbon atoms wherein 1 or 2 non-adjacent CH₂ groups of the divalent alkylene chain may be replaced independently from each other by —N(H)—, —N(C₁₋₆-alkyl)-, —N(—C(═O)—C₁₋₄-alkyl)-, —O— —wherein that C₁₋₆-alkyl and C₁₋₄-alkyl radicals may be straight-chain or branched—and wherein 2 adjacent CH₂ groups may together be replaced by a —CH═CH— moiety, which divalent alkylene chain may be unsubstituted or mono- or disubstituted with independently from each other straight-chain or branched —C₁₋₆-alkyl or ═O (oxo);
R^{Y4}, R^{Y5}, R^{Y6} denote independently from each other H, Hal, LA^{Y}, CA^{Y}, —CN, —NO₂, —SO₂NH₂, —SO₂NHR⁷, —SO₂NR^{Y7}R^{Y8}, —NH—SO₂—R^{Y9}, —NR^{Y7}—SO₂—R^{Y9}, —S—R^{Y9}, —S(═O)—R^{Y9}, —SO₂—R^{Y9}, —NH₂, —NHR^{Y7}, —NR^{Y7}R^{Y8}, —OH, —O—R^{Y9}, —CHO, —C(═O)—R^{Y9}, —COOH, —C(═O)—O—R^{Y9}, —C(═O)—NH₂, —C(═O)—NHR^{Y7}, —C(═O)—NR^{Y7}R^{Y8}, —NH—(C₁₋₃-alkylene)-C(═O)—NH₂, —NH—(C₁₋₃-alkylene)-C(═O)—NHR^{Y7}, —NH—(C₁₋₃-alkylene)-C(═O)—NR^{Y7}R^{Y8}, —NH—C(═O)—R^{Y9}, —NR^{Y7}—C(═O)—R^{Y9}, oxo (═O);
LA^{X} denotes straight-chain or branched C₁₋₆-alkyl which may be unsubstituted or mono-, di- or trisubstituted with independently from each other Hal, —CN, —NO₂, —SO₂NH₂, —SO₂NHR^{X7}, —SO₂NR^{X7}R^{X8}, —NH—SO₂—R^{X9}, —NR^{X7}—SO₂—R^{X9}, —S—R^{X9}, —S(═O)—R^{X9}, —SO₂—R^{X9}, —NH₂, —NHR^{X7}, —NR^{X7}R^{X8}, —OH, —O—R^{X9}, —CHO, —C(═O)—R^{X9}, —COOH, —C(═O)—O—R^{X9}, —C(═O)—NH₂, —C(═O)—NHR^{X7}, —C(═O)—NR^{X7}R^{X8}, —NH—(C₁₋₃-alkylene)-C(═O)—NH₂, —NH—(C₁₋₃-alkylene)-C(═O)—NHR^{X7}, —NH—(C₁₋₃-alkylene)-C(═O)—NR^{X7}R^{X8}, —NH—C(═O)—R^{X9}, —NR^{X7}—C(═O)—R^{X9}, oxo (═O), wherein 1 or 2 non-adjacent CH₂ groups of the C₁₋₆-alkyl radical may independently from each other be replaced by O, S, N(H) or N—R^{X7} and/or 1 or 2 non-adjacent CH groups of the C₁₋₆-alkyl radical may independently from each other be replaced by N;
LA^{Y} denotes straight-chain or branched C₁₋₆-alkyl which may be unsubstituted or mono-, di- or trisubstituted with independently from each other Hal, —CN, —NO₂, —SO₂NH₂, —SO₂NHR^{Y7}, —SO₂NR^{Y7}R^{Y8}, —NH—SO₂—R^{Y9}, —NR^{Y}—SO₂—R^{Y9}, —S—R^{Y9}, S(═O)—R^{Y9}, —SO₂—R^{Y9}, —NH₂, —NHR^{Y7}, —NR^{Y7}R^{Y8}, —OH, —O—R^{Y9}, —CHO, —C(═O)—R^{Y9}, —COOH, —C(═O)—O—R^{Y9}, —C(═O)—NH₂, —C(═O)—NHR^{Y7}, —C(═O)—NR^{Y7}R^{Y8}, —NH—(C₁₋₃-alkylene)-C(═O)—NH₂, —NH—(C₁₋₃-alkylene)-C(═O)—NHR^{Y7}, —NH—(C₁₋₃-alkylene)-C(═O)—NR^{Y7}R^{Y8}, —NH—C(═O)—R^{Y9}, —NR^{Y7}—C(═O)—R^{Y9}, oxo (═O), wherein 1 or 2 non-adjacent CH₂ groups of the C₁₋₆-alkyl radical may independently from each other be replaced by O, S, N(H) or N—R^{Y7} and/or 1 or 2 non-adjacent CH groups of the C₁₋₆-alkyl radical may independently from each other be replaced by N;
LA^{Z} denotes a divalent straight-chain or branched C₁₋₆-alkylene radical which alkylene radical may be unsubstituted or mono-, di- or trisubstituted with independently from each other Hal, —CN, —NO₂, —SO₂NH₂, —SO₂NHR^{Z7}, —SO₂NR^{Z7}R^{Z8}, —NH—SO₂—R^{Z9}, —NR^{Z7}—SO₂—R^{Z9}, —S—R^{Z9}, —S(═O)—R^{Z9}, —SO₂—R^{Z9}, —NH₂, —NHR^{Z7}, —NR^{Z7}R^{Z8}, —OH, —O—R^{Z9}, —CHO, —C(═O)—R^{Z9}, —COOH, —C(═O)—O—R^{Z9}, —C(═O)—NH₂, —C(═O)—NHR^{Z7}, —C(═O)—NR^{Z7}R^{Z8}, —NH—(C₁₋₃-alkylene)-C(═O)—NH₂, —NH—(C₁₋₃-alkylene)-C(═O)—NHR^{Z7}, —NH—(C₁₋₃-alkylene)-C(═O)—NR^{Z7}R^{Z8}, —NH—C(═O)—R^{Z9}, —NR^{Z7}—C(═O)—R^{Z9}, oxo (═O), wherein 1 or 2 non-adjacent CH₂ groups of that divalent alkylene radical may be replaced independently from each other by O, S or —N(H) and/or 1 or 2 non-adjacent CH groups of that divalent alkylene radical may be replaced by N;
R^{X7}, R^{X8}, R^{Y7}, R^{Y8}, R^{Z7}, R^{Z8} denote independently from each other straight-chain or branched C₁₋₆-alkyl, phenyl, a mono- or bicyclic aromatic ring system with 5, 6, 7, 8, 9, 10, 11 ring atoms wherein 1, 2, 3, 4 of said ring atoms is/are a hetero atom(s) selected from N, O and/or S and the remaining are carbon atoms and wherein that aromatic ring system may be unsubstituted or mono- or disubstituted with independently from each other straight-chain or branched C₁₋₆-alkyl or —O—C₁₋₆-alkyl or —NH₂, or a saturated monocyclic carbocycle with 3, 4, 5, 6, 7 carbon atoms
or
each pair R^{X7} and R^{X8}; R^{Y7} and R^{Y8}; R^{Z7} and R^{Z8} form independently from each other pair together with the nitrogen atom to which they are attached to a 3, 4, 5, 6 or 7 membered heterocycle wherein that heterocycle may not contain any further heteroatom or may contain besides said nitrogen atom one further hetero ring atom selected from N, O and S, wherein, if that further hetero atom is N, that further N may be substituted with H or straight-chain or branched C₁₋₆-alkyl;
R^{X9}, R^{Y9}, R^{Z9} denote independently from each other straight-chain or branched —C₁₋₆-alkyl, which may be unsubstituted or mono-, di- or trisubstituted with Hal, phenyl, a mono- or bicyclic aromatic ring system with 5, 6, 7, 8, 9, 10, 11 ring atoms wherein 1, 2, 3, 4 of said ring atoms is/are a hetero atom(s) selected from N, O and/or S and the remaining are carbon atoms and wherein that aromatic ring system may be unsubstituted or mono- or disubstituted with independently from each other straight-chain or branched C₁₋₆-alkyl or —O—C₁₋₆-alkyl or —NH₂, or a saturated monocyclic carbocycle with 3, 4, 5, 6, 7 carbon atoms;
R^{A1}, R^{A2}, R^{A3} denote independently from each other H, Hal, Ar^{X}, Hetar^{X}, Hetcyc^{X}, LA^{X}, CA^{X}, —CN, —NO₂, —SO₂NH₂, —SO₂NHR^{X7}, —SO₂NR^{X7}R^{X8}, —NH—SO₂—R^{X9}, —NR^{X7}—SO₂—R^{X9}, —S—R^{X9}, —S(═O)—R^{X9}, —SO₂—R^{X9}, —NH₂, —NHR^{X7}, —NR^{X7}R⁸, —OH, —O—R^{X9}, —CHO, —C(═O)—R^{X9}, —COOH, —C(═O)—O—R^{X9}, —C(═O)—NH₂, —C(═O)—NHR^{X7}, —C(═O)—NR^{X7}R^{X8}, —NH—(C₁₋₃-alkylene)-C(═O)—NH₂, —NH—(C₁₋₃-alkylene)-C(═O)—NHR^{X7}, —NH—(C₁₋₃-alkylene)-C(═O)—NR^{X7}R^{X8}, —NH—C(═O)—R^{X9}, —NR^{X7}—C(═O)—R^{X9}, oxo (═O);
or
two of R^{A1}, R^{A2} and R^{A3} form together with one carbon atom of that ring system A to which they both are attached to a saturated or partially unsaturated ring system E which ring system E is mono- or bicyclic and has 3, 4, 5, 6, 7, 8, 9, 10 ring atoms and may contain no hetero atom or 1, 2, 3 hetero atom(s) independently from each other selected from N, O and/or S that ring system E may be unsubstituted or mono-, di- or trisubstituted with independently from each other R^{E1}, R^{E2}, R^{E3};
R^{D1}, R^{D2}, R^{D3}, R^{E1}, R^{E2}, R^{E3} denote independently from each other H, Hal, Ar^{X}, Hetar^{X}, Hetcyc^{X}, LA^{X}, CA^{X}, —CN, —NO₂, —SO₂NH₂, —SO₂NHR^{X7}, —SO₂NR^{X7}R^{X8}, —NH—SO₂—R^{X9}, —NR^{X7}—SO₂—R^{X9}, —S—R^{X9}, —S(═O)—R^{X9}, —SO₂—R^{X9}, —NH₂, —NHR^{X7}, —NR^{X7}R^{X8}, —OH, —O—R^{X9}, —CHO, —C(═O)—R^{X9}, —COOH, —C(═O)—O—R^{X9}, —C(═O)—NH₂, —C(═O)—NHR^{X7}, —C(═O)—NR^{X7}R^{X8}, —NH—(C₁₋₃-alkylene)-C(═O)—NH₂, —NH—(C₁₋₃-alkylene)-C(═O)—NHR^{X7}, —NH—(C₁₋₃-alkylene)-C(═O)—NR^{X7}R^{X8}, —NH—C(═O)—R^{X9}, —NR^{X7}—C(═O)—R^{X9}, oxo (═O);
R^{D4} denotes H, Hal, Ar^{X}, Hetar^{X}, Hetcyc^{X}, LA^{X}, CA^{X}, —CN, —NO₂, —SO₂NH₂, —SO₂NHR^{X7}, —SO₂NR^{X7}R^{X8}, —NH—SO₂—R^{X9}, —NR^{X7}—SO₂—R^{X9}, —S—R^{X9}, —S(═O)—R^{X9}, —SO₂—R^{X9}, —NH₂, —NHR^{X7}, —NR^{X7}R^{X8}, —OH, —O—R^{X9}, —CHO, —C(═O)—R^{X9}, —COOH, —C(═O)—O—R^{X9}, —C(═O)—NH₂, —C(═O)—NHR^{X7}, —C(═O)—NR^{X7}R^{X8}, —NH—(C₁₋₃-alkylene)-C(═O)—NH₂, —NH—(C₁₋₃-alkylene)-C(═O)—NHR^{X7}, —NH—(C₁₋₃-alkylene)-C(═O)—NR^{X7}R^{X8}, —NH—C(═O)—R^{X9}, —NR^{X7}—C(═O)—R^{X9};
CA^{X}, CA^{Y} denote independently from each other a saturated monocyclic carbocycle with 3, 4, 5, 6, 7 carbon atoms which carbocycle may be unsubstituted or mono- or disubstituted with independently from each other R^{CA1}, R^{CA2};
R^{CA1}, R^{CA2} denote independently from each other H, Hal, Ar^{X}, Ar^{X}—Ar^{Y}, Ar^{X}-Hetar^{Y}, Ar^{X}-Hetcyc^{Y}, Ar^{X}-LA^{Z}-Ar^{Y}, Ar^{X}-LA^{Z}-Hetar^{Y}, Ar^{X}-LA^{Z}-Hetcyc^{Y}, Hetar^{X}, Hetar^{X}-Ar^{Y}, Hetar^{X}-Hetar^{Y}, Hetar^{X}-Hetcyc^{Y}, Hetar^{X}-LA^{Z}-Ar^{Y}, Hetar^{X}-LA^{Z}-Hetar^{Y}, Hetar^{X}-LA^{Z}-Hetcyc^{Y}, Hetcyc^{X}, Hetcyc^{X}-Ar^{Y}, Hetcyc^{X}-Hetar^{Y}, Hetcyc^{X}-Hetcyc^{Y}, Hetcyc^{X}-LA^{Z}-Ar^{Y}, Hetcyc^{X}-LA^{Z}-Hetar^{Y}, Hetcyc^{X}-LA^{Z}-Hetcyc^{Y}, LA^{X}, LA^{Z}-Ar^{Y}, LA^{Z}-Hetar^{Y}, LA^{Z}-Hetcyc^{Y}, —CN, —NO₂, —SO₂NH₂, —SO₂NHR^{X7}, —SO₂NR^{X7}R^{X8}, —NH—SO₂—R^{X9}, —NR^{X7}—SO₂—R^{X9}, —S—R^{X9}, S(═O)—R^{X9}, —SO₂—R^{X9}, —NH₂, —NHR^{X7}, —NR^{X7}R^{X8}, —OH, —O—R^{X9}, —CHO, —C(═O)—R^{X9}, —COOH, —C(═O)—O—R^{X9}, —C(═O)—NH₂, —C(═O)—NHR^{X7}, —C(═O)—NR^{X7}R^{X8}, —NH—(C₁₋₃-alkylene)-C(═O)—NH₂, —NH—(C₁₋₃-alkylene)-C(═O)—NHR^{X7}, —NH—(C₁₋₃-alkylene)-C(═O)—NR^{X7}R^{X8}, —NH—C(═O)—R^{X9}, —NR^{X7}—C(═O)—R^{X9}, oxo (═O),
   with the proviso that if R^{CA1} or R^{CA2} denotes Ar^{X}, Ar^{X}—Ar^{Y}, Ar^{X}-Hetar^{Y}, Ar^{X}-Hetcyc^{Y}, Ar^{X}-LA^{Z}-Ar^{Y}, Ar^{X}-LA^{Z}-Hetar^{Y}, Ar^{X}-LA^{Z}-Hetcyc^{Y}, Hetar^{X}, Hetar^{X}-Ar^{Y}, Hetar^{X}-Hetar^{Y}, Hetar^{X}-Hetcyc^{Y}, Hetar^{X}-LA^{Z}-Ar^{Y}, Hetar^{X}-LA^{Z}-Hetar^{Y}, Hetar^{X}-LA^{Z}-Hetcyc^{Y}, Hetcyc^{X}, Hetcyc^{X}-Ar^{Y}, Hetcyc^{X}-Hetar^{Y}, Hetcyc^{X}-Hetcyc^{Y}, Hetcyc^{X}-LA^{Z}-Ar^{Y}, Hetcyc^{X}-LA^{Z}-Hetar^{Y}, Hetcyc^{X}-LA^{Z}-Hetcyc^{Y}, LA^{Z}-Ar^{Y}, LA^{Z}-Hetar^{Y}, LA^{Z}-Hetcyc^{Y}, then Ar^{X}, Ar^{Y}, Hetar^{X}, Hetar^{Y}, Hetcyc^{X}, Hetcyc^{Y} may not be substituted with CA^{X} or CA^{Y};
Hal denotes F, Cl, Br, I;
or derivatives, N-oxides, prodrugs, solvates, tautomers or stereoisomers thereof as well as the physiologically acceptable salts of each of the foregoing, including mixtures thereof in all ratios.

It is to be noted with regard to ring system A as defined hereinbefore and hereinafter that said mono- or bicyclic ring system A may be saturated or partially unsaturated. In this context, the term “partially unsaturated” refers either to (i) a mono- or bicyclic ring system with one or more sites of unsaturation, however, without being aromatic (also referred to as “non-aromatic partially unsaturated”); or to (ii) a bicyclic ring system with one ring being a non-aromatic carbocyclic (cycloaliphatic) or heterocyclic ring and the other ring being an aromatic (aryl) or heteroaromatic (heteroaryl) ring fused to that non-aromatic ring (also referred to as “partially aromatic”). Examples for such a partially aromatic ring system A may be tetrahydronaphthalinyl (tetralinyl), 1,2- or 1,4-dihydrobenzopyranyl and tetrahydroquinolinyl. If ring system A denotes such a partially aromatic ring system, it is attached to the rest of the molecule (its pendant group) via the non-aromatic ring.

Likewise, it is to be noted with regard to ring systems D and E, respectively, as defined hereinbefore and hereinafter that said mono- or bicyclic ring system D or E may be saturated or partially unsaturated. In this context, the term “partially unsaturated” refers either to (i) a mono- or bicyclic ring system with one or more sites of unsaturation, however, without being aromatic (also referred to as “non-aromatic partially unsaturated”); or to (ii) a bicyclic ring system with one ring being a non-aromatic carbocyclic (cycloaliphatic) or heterocyclic ring and the other ring being an aromatic (aryl) or heteroaromatic (heteroaryl) ring fused to that non-aromatic ring (also referred to as “partially aromatic”). Examples for such a partially aromatic ring system D or E may be tetrahydronaphthalinyl (tetralinyl) and tetrahydroquinolinyl. If ring system D or E denotes such a partially aromatic ring system, it is attached to the rest of the molecule (its pendant group) via the non-aromatic ring.

Furthermore, it is to be noted that if a ring system E is present in a compound of the present invention, it is connected to ring system A to form a spiro ring system, which means a bicyclic moiety is formed by both ring systems which are connected through just one, i.e. the same, atom (also referred to as “spiro atom”) which is shared by both ring systems.

In general, all residues which occur more than once may be identical or different, i.e. are independent of one another. Above and below, the residues and parameters have the meanings indicated for formula (I), unless expressly indicated otherwise. Accordingly, the invention relates, in particular, to the compounds of formula (I) in which at least one of the said residues has one of the preferred meanings indicated below.

Any of those preferred or particular embodiments of the present invention as specified below and in the claims do not only refer to the specified compounds of formula (I) but to derivatives, N-oxides, prodrugs, solvates, tautomers or stereoisomers thereof as well as the physiologically acceptable salts of each of the foregoing, including mixtures thereof in all ratios, too, unless indicated otherwise.

In a particular embodiment, PE1, the compounds of the present invention are compounds of formula (I)

wherein
X denotes N—R⁷ or O;
R¹ denotes Ar^{X}, Ar^{X}-Hetar^{Y}, Ar^{X}-Hetcyc^{Y}, Hetar^{X}, Hetcyc^{X}, Hetar^{X}-LA^{Z}-Ar^{Y};
R² and R³ denote independently from each other H, —OH, unsubstituted straight-chain or branched —C₁₋₆-alkyl, unsubstituted straight-chain or branched —O—C₁₋₆-alkyl, Hal, —CN, —C(═O)—NH₂;
R⁴ denotes Ar^{X}, Ar^{X}—Ar^{Y}, Ar^{X}-Hetar^{Y}, Ar^{X}-Hetcyc^{Y}, Hetar^{X}, Hetar^{X}-Ar^{Y}, Hetar^{X}-Hetar^{Y}, Hetar^{X}-Hetcyc^{Y}, Hetcyc^{X}, Hetcyc^{X}-Hetar^{Y}, Hetcyc^{X}-LA^{Z}-Ar^{Y}, LA^{X}, LA^{Z}-Hetar^{Y}, LA^{Z}-Hetcyc^{Y};
R⁵ denotes H, Hetar^{X}, Hetcyc^{X}, LA^{X}, CA^{X}, —C(═O)—NR^{X7}R^{X8};
or
R⁴ and R⁵ form together with the carbon atom to which they are attached to a saturated or partially unsaturated ring system A which ring system A is mono- or bicyclic and has 3, 4, 5, 6, 7, 8, 9, 10 ring atoms and may contain no hetero ring atom or 1, 2, 3 hetero ring atom(s) independently from each other selected from N, O and/or S that ring system A may be unsubstituted or mono-, di- or trisubstituted with independently from each other R^{A1}, R^{A2}, R^{A3};
R⁶ denotes denotes H, Hetar^{X}, Hetcyc^{X}, LA^{X};
or
R⁵ and R⁶ form together with the carbon atom to which they are attached to a saturated or partially unsaturated ring system D which ring system D is mono- or bicyclic and has 3, 4, 5, 6, 7, 8, 9, 10 ring atoms and may contain no hetero ring atom or 1, 2, 3 hetero ring atom(s) independently from each other selected from N, O and/or S that ring system D may be unsubstituted or mono-, di- or trisubstituted with independently from each other R^{D1}, R^{D2}, R^{D3};
or
R⁵ and R⁶ form together with the carbon atom to which they are attached to a C═CHR^{D4} moiety;
R⁷ denotes H, Hetar^{X}, Hetcyc^{X}, LA^{X};
Ar^{X} denotes a mono-, bi- or tricyclic aromatic ring system with 5, 6, 7, 8, 9, 10, 11, 12, 13, 14 ring carbon atoms which ring system may be unsubstituted or mono-, di- or trisubstituted with independently from each other R^{X1}, R^{X2}, R^{X3};
Ar^{Y} denotes a mono-, bi- or tricyclic aromatic ring system with 5, 6, 7, 8, 9, 10, 11, 12, 13, 14 ring carbon atoms which ring system may be unsubstituted or mono-, di- or trisubstituted with independently from each other R^{Y1}, R^{Y2}, R^{Y3};
Hetar^{X} denotes a mono, bi- or tricyclic aromatic ring system with 5, 6, 7, 8, 9, 10, 11, 12, 13, 14 ring atoms wherein 1, 2, 3, 4, 5 of said ring atoms is/are a hetero atom(s) selected from N, O and/or S and the remaining are carbon atoms, wherein that aromatic ring system may be unsubstituted or mono-, di- or trisubstituted with independently from each other R^{X1}, R^{X2}, R^{X3};
Hetar^{Y} denotes a mono, bi- or tricyclic aromatic ring system with 5, 6, 7, 8, 9, 10, 11, 12, 13, 14 ring atoms wherein 1, 2, 3, 4, 5 of said ring atoms is/are a hetero atom(s) selected from N, O and/or S and the remaining are carbon atoms, wherein that aromatic ring system may be unsubstituted or mono-, di- or trisubstituted with independently from each other R^{Y1}, R^{Y2}, R^{Y3};
Hetcyc^{X} denotes a saturated or partially unsaturated mono-, bi- or tricyclic heterocycle with 3, 4, 5, 6, 7, 8, 9, 10, 11, 12, 13, 14 ring atoms wherein 1, 2, 3, 4, 5 ring atom(s) is/are heteroatom(s) selected from N, O and/or S and the remaining ring atoms are carbon atoms, wherein that heterocycle may be unsubstituted or mono-, di- or trisubstituted with R^{X4}, R^{X5}, R^{X6};
Hetcyc^{Y} denotes a saturated or partially unsaturated mono-, bi- or tricyclic heterocycle with 3, 4, 5, 6, 7, 8, 9, 10, 11, 12, 13, 14 ring atoms wherein 1, 2, 3, 4, 5 ring atom(s) is/are heteroatom(s) selected from N, O and/or S and the remaining ring atoms are carbon atoms, wherein that heterocycle may be unsubstituted or mono-, di- or trisubstituted with R^{Y4}, R^{Y5}, R^{Y6};
R^{X1}, R^{X2}, R^{X3} denote independently from each other H, Hal, LA^{X}, —CN, —NO₂, —SO₂NH₂, —SO₂NHR^{X7}, —SO₂NR^{X7}R^{X8}, —NH—SO₂—R^{X9}, —NR^{X7}—SO₂—R^{X9}, —SO₂—R^{X9}, —NH₂, —NHR^{X7}, —NR^{X7}R^{X8}, —OH, —O—R^{X9}, —CHO, —C(═O)—R^{X9}, —COOH, —C(═O)—O—R^{X9}, —C(═O)—NH₂, —C(═O)—NHR^{X7}, —C(═O)—NR^{X7}R^{X8}, —NH—(C₁₋₃-alkylene)-C(═O)—NH₂, —NH—(C₁₋₃-alkylene)-C(═O)—NHR^{X7}, —NH—(C₁₋₃-alkylene)-C(═O)—NR^{X7}R^{X8}, —NH—C(═O)—R^{X9}, —NR^{X7}—C(═O)—R^{X9}
or
two of R^{X1}, R^{X2}, R^{X3} form a divalent alkylene chain with 3, 4, 5 chain carbon atoms wherein 1 or 2 of non-adjacent CH₂ groups of the divalent alkylene chain may be replaced independently from each other by —N(H)—, —N(C₁₋₆-alkyl)-, —N(—C(═O)—C₁₋₄-alkyl)-, —O— —wherein that C₁₋₆-alkyl and C₁₋₄-alkyl radicals may be straight-chain or branched—and wherein 2 adjacent CH₂ groups may together be replaced by a —CH═CH— moiety, which divalent alkylene chain may be unsubstituted or mono- or disubstituted with independently from each other straight-chain or branched —C₁₋₆-alkyl or ═O (oxo);
R^{X4}, R^{X5}, R^{X6} denote independently from each other H, Hal, LA^{X}, —CN, —NO₂, —SO₂NH₂, —SO₂NHR^{X7}, —SO₂NR^{X7}R^{X8}, —NH—SO₂—R^{X9}, —NR^{X7}—SO₂—R^{X9}, —SO₂—R^{X9}, —NH₂, —NHR⁷, —NR^{X7}R^{X8}, —OH, —O—R^{X9}, —CHO, —C(═O)—R^{X9}, —COOH, —C(═O)—O—R^{X9}, —C(═O)—NH₂, —C(═O)—NHR^{X7}, —C(═O)—NR^{X7}R^{X8}, —NH—(C₁₋₃-alkylene)-C(═O)—NH₂, —NH—(C₁₋₃-alkylene)-C(═O)—NHR^{X7}, —NH—(C₁₋₃-alkylene)-C(═O)—NR^{X7}R^{X8}, —NH—C(═O)—R^{X9}, —NR^{X7}—C(═O)—R^{X9}, oxo (═O);
R^{Y1}, R^{Y2}, R^{Y3} denote independently from each other H, Hal, LA^{Y}, —CN, —NO₂, —SO₂NH₂, —SO₂NHR^{Y7}, —SO₂NR^{Y7}R^{Y8}, —NH—SO₂—R^{Y9}, —NR^{Y7}—SO₂—R^{Y9}, —SO₂—R^{Y9}, —NH₂, —NHR^{Y7}, —NR^{Y7}R^{Y8}, —OH, —O—R^{Y9}, —CHO, —C(═O)—R^{Y9}, —COOH, —C(═O)—O—R^{Y9}, —C(═O)—NH₂, —C(═O)—NHR^{Y7}, —C(═O)—NR^{Y7}R^{Y8}, —NH—(C₁₋₃-alkylene)-C(═O)—NH₂, —NH—(C₁₋₃-alkylene)-C(═O)—NHR^{Y7}, —NH—(C₁₋₃-alkylene)-C(═O)—NR^{Y7}R^{Y8}, —NH—C(═O)—R^{Y9}, —NR^{Y7}—C(═O)—R^{Y9}
or
two of R^{Y1}, R^{Y2}, R^{Y3} form a divalent alkylene chain with 3, 4, 5 chain carbon atoms wherein 1 or 2 of non-adjacent CH₂ groups of the divalent alkylene chain may be replaced independently from each other by —N(H)—, —N(C₁₋₆-alkyl)-, —N(—C(═O)—C₁₋₄-alkyl)-, —O— —wherein that C₁₋₆-alkyl and C₁₋₄-alkyl radicals may be straight-chain or branched—and wherein 2 adjacent CH₂ groups may together be replaced by a —CH═CH— moiety, which divalent alkylene chain may be unsubstituted or mono- or disubstituted with independently from each other straight-chain or branched —C₁₋₆-alkyl or ═O (oxo);
R^{Y4}, R^{Y5}, R^{Y6} denote independently from each other H, Hal, LA^{Y}, —CN, —NO₂, —SO₂NH₂, —SO₂NHR^{Y7}, —SO₂NR^{Y7}R^{Y8}, —NH—SO₂—R^{Y9}, —NR^{Y7}—SO₂—R^{Y9}, —SO₂—R^{Y9}, —NH₂, —NHR^{Y7}, —NR^{Y7}R^{Y8}, —OH, —O—R^{Y9}, —CHO, —C(═O)—R^{Y9}, —COOH, —C(═O)—O—R^{Y9}, —C(═O)—NH₂, —C(═O)—NHR^{Y7}, —C(═O)—NR^{Y7}R^{Y8}, —NH—(C₁₋₃-alkylene)-C(═O)—NH₂, —NH—(C₁₋₃-alkylene)-C(═O)—NHR^{Y7}, —NH—(C₁₋₃-alkylene)-C(═O)—NR^{Y7}R^{Y8}, —NH—C(═O)—R^{Y9}, —NR^{Y7}—C(═O)—R^{Y9}, oxo (═O);
LA^{X} denotes straight-chain or branched —C₁₋₆-alkyl which may be unsubstituted or mono-, di- or trisubstituted with independently from each other Hal, —CN, —NO₂, —SO₂NH₂, —SO₂NHR^{X7}, —SO₂NR^{X7}R⁸, —NH—SO₂—R^{X9}, —NR^{X7}—SO₂—R^{X9}, —SO₂—R^{X9}, —NH₂, —NHR^{X7}, —NR^{X7}R^{X8}, —OH, —O—R^{X9}, —CHO, —C(═O)—R^{X9}, —COOH, —C(═O)—O—R^{X9}, —C(═O)—NH₂, —C(═O)—NHR^{X7}, —C(═O)—NR^{X7}R^{X8}, —NH—(C₁₋₃-alkylene)-C(═O)—NH₂, —NH—(C₁₋₃-alkylene)-C(═O)—NHR^{X7}, —NH—(C₁₋₃-alkylene)-C(═O)—NR^{X7}R⁸, —NH—C(═O)—R^{X9}, —NR^{X7}—C(═O)—R^{X9}, oxo (═O), wherein 1 or 2 non-adjacent CH₂ groups of the C₁₋₆-alkyl radical may independently from each other be replaced by O, S, N(H) or N—R^{X7} and/or 1 or 2 non-adjacent CH groups of the C₁₋₆-alkyl radical may independently from each other be replaced by N;
LA^{Y} denotes straight-chain or branched C₁₋₆-alkyl which may be unsubstituted or mono-, di- or trisubstituted with independently from each other Hal, —CN, —NO₂, —SO₂NH₂, —SO₂NHR^{Y7}, —SO₂NR^{Y7}R^{Y8}, —NH—SO₂—R^{Y9}, —NR^{Y7}—SO₂—R^{Y9}, —SO₂—R^{Y9}, —NH₂, —NHR^{Y7}, —NR^{Y7}R^{Y8}, —OH, —O—R^{Y9}, —CHO, —C(═O)—R^{Y}, —COOH, —C(═O)—O—R^{Y9}, —C(═O)—NH₂, —C(═O)—NHR^{Y7}, —C(═O)—NR^{Y7}R^{Y8}, —NH—(C₁₋₃-alkylene)-C(═O)—NH₂, —NH—(C₁₋₃-alkylene)-C(═O)—NHR^{Y7}, —NH—(C₁₋₃-alkylene)-C(═O)—NR^{Y7}R^{Y8}, —NH—C(═O)—R^{Y9}, —NR^{Y7}—C(═O)—R^{Y9}, oxo (═O), wherein 1 or 2 non-adjacent CH₂ groups of the alkyl chain may independently from each other be replaced by O, S, N(H) or N—R^{Y7} and/or 1 or 2 non-adjacent CH groups of the alkyl chain may independently from each other be replaced by N;
LA^{Z} denotes a divalent straight-chain or branched C₁₋₆-alkylene radical which divalent alkylene radical may be unsubstituted or mono-, di- or trisubstituted with independently from each other Hal, —CN, —NO₂, —SO₂NH₂, —SO₂NHR^{Z7}, —SO₂NR^{Z7}R^{Z8}, —NH—SO₂—R^{Z9}, —NR^{Z7}—SO₂—R^{Z9}, —SO₂—R^{Z9}, —NH₂, —NHR^{Z7}, —NR^{Z7}R^{Z8}, —OH, —O—R^{Z9}, —CHO, —C(═O)—R^{Z9}, —COOH, —C(═O)—O—R^{Z9}, —C(═O)—NH₂, —C(═O)—NHR^{Z7}, —C(═O)—NR^{Z7}R^{Z8}, —NH—(C₁₋₃-alkylene)-C(═O)—NH₂, —NH—(C₁₋₃-alkylene)-C(═O)—NHR^{Z7}, —NH—(C₁₋₃-alkylene)-C(═O)—NR^{Z7}R^{Z8}, —NH—C(═O)—R^{Z9}, —NR^{Z7}—C(═O)—R^{Z9}, oxo (═O), wherein 1 or 2 non-adjacent CH₂ groups of the divalent alkylene radical may be replaced independently from each other by O, S or N(H) and/or 1 or 2 non-adjacent CH groups of the divalent alkylene radical may be replaced by N;
CA^{X} denotes a saturated monocyclic carbocycle with 3, 4, 5, 6, 7 carbon atoms which carbocycle may be unsubstituted or mono- or disubstituted with independently from each other R^{CA1}, R^{CA2};
R^{X7}, R^{X8}, R^{X9}, R^{Y7}, R^{Y8}, R^{Y9}, R^{Z7}, R^{Z8}, R^{Z9} denote independently from each other straight-chain or branched C₁₋₆-alkyl, phenyl, a monocyclic aromatic ring system with 5, 6, 7 ring atoms wherein 1, 2, 3, 4 of said ring atoms is/are a hetero atom(s) selected from N, O and/or S and the remaining are carbon atoms and wherein that aromatic ring system may be unsubstituted or mono- or disubstituted with independently from each other straight-chain or branched C₁₋₆-alkyl, or a saturated monocyclic carbocycle with 3, 4, 5, 6, 7 carbon atoms
or
each pair R^{X7} and R^{X8}; R^{Y7} and R^{Y8}; R^{Z7} and R^{Z8} form independently from each other pair together with the nitrogen atom to which they are attached to a 3, 4, 5, 6 or 7 membered heterocycle wherein that heterocycle may not contain any further heteroatom or may contain besides said nitrogen atom one further hetero ring atom selected from N, O and S, wherein, if that further hetero atom is N, that further N may be substituted with H or straight-chain or branched C₁₋₆-alkyl;
R^{X9}, R^{Y9}, R^{Z9} denote independently from each other straight-chain or branched —C₁₋₆-alkyl, which may be unsubstituted or mono-, di- or trisubstituted with Hal, phenyl, a monocyclic aromatic ring system with 5, 6, 7 ring atoms wherein 1, 2, 3, 4 of said ring atoms is/are a hetero atom(s) selected from N, O and/or S and the remaining are carbon atoms and wherein that aromatic ring system may be unsubstituted or mono- or disubstituted with independently from each other straight-chain or branched C₁₋₆-alkyl, or a saturated monocyclic carbocycle with 3, 4, 5, 6, 7 carbon atoms;
R^{A1}, R^{A2}, R^{A3} denote independently from each other H, Hal, LA^{X}, Ar^{X}, Hetar^{X}, —CN, —NO₂, —SO₂NH₂, —SO₂NHR^{X7}, —SO₂NR^{X7}R^{X8}, —NH—SO₂—R^{X9}, —NR^{X7}—SO₂—R^{X9}, —SO₂—R^{X9}, —NH₂, —NHR^{X7}, —NR^{X7}R^{X8}, —OH, —O—R^{X9}, —CHO, —C(═O)—R^{X9}, —COOH, —C(═O)—O—R^{X9}, —C(═O)—NH₂, —C(═O)—NHR^{X7}, —C(═O)—NR^{X7}R^{X8}, —NH—(C₁₋₃-alkylene)-C(═O)—NH₂, —NH—(C₁₋₃-alkylene)-C(═O)—NHR^{X7}, —NH—(C₁₋₃-alkylene)-C(═O)—NR^{X7}R^{X8}, —NH—C(═O)—R^{X9}, —NR^{X7}—C(═O)—R^{X9}, oxo (═O);
or
two of R^{A1}, R^{A2} and R^{A3} form together with one carbon atom of that ring system A to which they both are attached to a saturated or partially unsaturated ring system E which ring system E is mono- or bicyclic and has 3, 4, 5, 6, 7, 8, 9, 10 ring atoms and may contain no hetero atom or 1, 2, 3 hetero atom(s) independently from each other selected from N, O and/or S that ring system E may be unsubstituted or mono-, di- or trisubstituted with independently from each other R^{E1}, R^{E2}, R^{E3};
R^{D1}, R^{D2}, R^{D3}, R^{E1}, R^{E2}, R^{E3} denote independently from each other H, Hal, LA^{X}, —CN, —NO₂, —SO₂NH₂, —SO₂NHR⁷, —SO₂NR^{X7}R^{X8}, —NH—SO₂—R^{X9}, —NR^{X7}—SO₂—R^{X9}, —SO₂—R^{X9}, —NH₂, —NHR^{X7}, —NR^{X7}R^{X8}, —OH, —O—R^{X9}, —CHO, —C(═O)—R^{X9}, —COOH, —C(═O)—O—R^{X9}, —C(═O)—NH₂, —C(═O)—NHR^{X7}, —C(═O)—NR^{X7}R^{X8}, —NH—(C₁₋₃-alkylene)-C(═O)—NH₂, —NH—(C₁₋₃-alkylene)-C(═O)—NHR^{X7}, —NH—(C₁₋₃-alkylene)-C(═O)—NR^{X7}R^{X8}, —NH—C(═O)—R^{X9}, —NR^{X7}—C(═O)—R^{X9}, oxo (═O);
R^{D4} denotes —COOH;
R^{CA1}, R^{CA2} denote independently from each other H, Hal, R^{X9}, —CN, —NO₂, —SO₂NH₂, —SO₂NHR^{X7}, —SO₂NR^{X7}R^{X8}, —NH—SO₂—R^{X9}, —NR^{X7}—SO₂—R^{X9}, —S—R^{X9}, S(═O)—R^{X9}, —SO₂—R^{X9}, —NH₂, —NHR^{X7}, —NR^{X7}R^{X8}, —OH, —O—R^{X9}, —CHO, —C(═O)—R^{X9}, —COOH, —C(═O)—O—R^{X9}, —C(═O)—NH₂, —C(═O)—NHR^{X7}, —C(═O)—NR^{X7}R^{X8}, —NH—(C₁₋₃-alkylene)-C(═O)—NH₂, —NH—(C₁₋₃-alkylene)-C(═O)—NHR^{X7}, —NH—(C₁₋₃-alkylene)-C(═O)—NR^{X7}R^{X8}, —NH—C(═O)—R^{X9}, —NR^{X7}—C(═O)—R^{X9}, oxo (═O);
Hal denotes F, Cl, Br, I.

In another particular embodiment, PE1a, of the present invention—which may also be an embodiment of particular embodiment PE1—the substituent R¹, that denotes Ar^{X}, Ar^{X}-Hetar^{Y}, Ar^{X}-Hetcyc^{Y}, Hetar^{X}, Hetcyc^{X}, Hetcyc^{X}, Hetar^{X}-LA^{Z}-Ar^{Y}, is attached to the core quinoxaline ring system of formula (I) via a ring carbon atom.

A further particular embodiment of the present invention, PE2, which may optionally be part of the above described particular embodiments PE1 and/or PE1a, comprises compounds of formula (I) wherein
R² denotes H, unsubstituted straight-chain or branched —C₁₋₆-alkyl, OH, —CN; preferably, it denotes H;
R³ denotes H, unsubstituted straight-chain or branched —C₁₋₆-alkyl, OH; preferably, it denotes H.

Another particular embodiment of the present invention, PE3, which may optionally be part of the above described particular embodiments PE1, PE1a and/or PE2, comprises compounds of formula (I) wherein
X denotes N—R⁷ or O; preferably, it denotes NR⁷;
R⁷ denotes H or straight-chain or branched C₁₋₆-alkyl or Hetar^{X}; preferably, it denotes H.

In a preferred embodiment, PE3a, of this particular embodiment PE3 the compounds of present invention of formula (I) are those wherein
both R² and R³ denote H (see PE2).

If, however, R⁷ denotes Hetar^{X} it is then preferred that
Hetar^{X} denotes a monocyclic aromatic ring system with 5, 6, 7 ring atoms wherein 1, 2, 3, 4 of said ring atoms is/are a hetero atom(s) selected from N, O and/or S and the remaining are carbon atoms, wherein that aromatic ring system may be unsubstituted or mono- or disubstituted with independently from each other R^{X71a}, R^{X72a}; preferably triazolyl or pyridinyl, each of which is unsubstituted or monosubstituted with R^{X79}, —C(═O)—NH₂, —SO₂—R^{X79};
R^{X71a}, R^{X72a} denotes independently from each other Hal, R^{X79}, —CN, —NO₂, —SO₂NH₂, —SO₂NHR^{X77}, —SO₂NR^{X77}R^{X78}, —NH—SO₂—R^{X79}, —NR^{X77}—SO₂—R^{X79}, —SO₂—R^{X79}, —NH₂, —NHR^{X77}, —NR^{X77}R^{X78}, —OH, —O—R^{X79}, —CHO, —C(═O)—R^{X79}, —COOH, —C(═O)—O—R^{X79}, —C(═O)—NH₂, —C(═O)—NHR^{X77}, —C(═O)—NR^{X77}R^{X78}, —NH—C(═O)—R^{X79}, —NR^{X77}—C(═O)—R^{X79};
R^{X77}, R^{X78}, R^{X79} denote independently from each other straight-chain or branched C₁₋₆-alkyl or a saturated monocyclic carbocycle with 3, 4, 5, 6, 7 carbon atoms
or
R^{X77} and R^{X78} form together with the nitrogen atom to which they are attached to a 3, 4, 5, 6 or 7 membered heterocycle wherein that heterocycle may not contain any further heteroatom or may contain besides said nitrogen atom one further hetero ring atom selected from N, O and S, wherein, if that further hetero atom is N, that further N may be substituted with H or straight-chain or branched C₁₋₆-alkyl.

Still another particular embodiment of the present invention, PE4, which may also be part of other particular embodiments PE1, PE1a, PE2, PE3, PE3a, comprises compounds wherein
R¹ denotes Ar^{X}, Hetar^{X} or Hetar^{X}-LA^{Z}-Ar^{Y}.

In a preferred embodiment, PE4a, of this particular embodiment PE4
R¹ denotes Ar^{X1}, Hetar^{X1} or Hetar^{X1}-LA^{Z}-Ar^{Y1};
Ar^{X1} denotes a mono- or bicyclic aromatic ring system with 6, 7, 8, 9, 10 ring carbon atoms which ring system may be unsubstituted or mono-, di- or trisubstituted with independently from each other R^{X1a}, R^{X2a}, R^{X3}a;
Ar^{Y1} denotes a mono- or bicyclic aromatic ring system with 6, 7, 8, 9, 10 ring carbon atoms which ring system may be unsubstituted or mono-, di- or trisubstituted with independently from each other R^{Y1a}, R^{Y2a}, R^{Y3a};
Hetar^{X1} denotes a mono or bicyclic aromatic ring system with 5, 6, 8, 9, 10 ring atoms wherein 1, 2, 3 of said ring atoms is/are a hetero atom(s) selected from N, O and/or S and the remaining are carbon atoms, wherein that aromatic ring system may be unsubstituted or mono-, di- or trisubstituted with independently from each other R^{X1b}, R^{X2b}, R^{X3b};
LA^{Z1} denotes a divalent straight-chain or branched C₁₋₆-alkylene radical;
R^{X1a}, R^{X2a}, R^{X3a}, R^{X1b}, R^{X2b}, R^{X3b}, R^{Y1a}, R^{Y2a}, R^{Y3a} denote independently from each other LA^{X1}, Br, —CN, —C(═O)—NH₂, —C(═O)—R^{X9a}, —NH₂, —NHR^{X7a}, —NR^{X7a}R^{X8a}, —NO₂, —OR^{X9a}
or
two of R^{X1a}, R^{X2a}, R^{X3a} form a divalent alkylene chain with 3, 4 or 5 chain carbon atoms wherein 1 or 2 of non-adjacent CH₂ groups of the divalent alkylene chain may be replaced independently from each other by —N(H)—, —N(C₁₋₆-alkyl)-, —N(—C(═O)—C₁₋₄-alkyl)-, —O— —wherein that C₁₋₆-alkyl and C₁₋₄-alkyl radicals may be straight-chain or branched—and wherein 2 adjacent CH₂ groups may together be replaced by a —CH═CH— moiety, which divalent alkylene chain may be unsubstituted or mono- or disubstituted with straight-chain or branched —C₁₋₆-alkyl and/or monosubstituted with ═O (oxo);
LA^{X1} denotes straight-chain or branched —C₁₋₆-alkyl which may be unsubstituted or monosubstituted with —OR^{X9a};
R^{X7a}, R^{X8a} denote independently from each other straight-chain or branched —C₁₋₆-alkyl or form together with the nitrogen atom to which they are attached to a 3, 4, 5, 6 or 7 membered heterocycle wherein that heterocycle may not contain any further heteroatom or may contain besides said nitrogen atom one further hetero ring atom selected from N, O and S, wherein, if that further hetero atom is N, that further N may be substituted with H or straight-chain or branched —C₁₋₆-alkyl;
R^{X9a} denotes straight-chain or branched —C₁₋₆-alkyl.

In a preferred particular embodiment, PE4b,
R¹ denotes Ar^{X1}, Hetar^{X1} or Hetar^{X1}-LA^{Z1}-Ar^{Y1}; in particular Hetar^{X1};
Ar^{X1} denotes phenyl or naphthyl which may be unsubstituted or mono- or disubstituted with R^{X1a}, R^{X2a};
Hetar^{X1} denotes (a) a monocyclic aromatic ring system with 6 ring atoms wherein 1 of said ring atoms is a nitrogen atom and the remaining are carbon atoms; or (b) a bicyclic aromatic ring system with 9 ring atoms wherein (i) 1 of said ring atoms is a nitrogen atom or an oxygen atom or a sulfur atom and the remaining are carbon atoms; or (ii) 2 of said ring atoms are nitrogen atoms and the remaining are carbon atoms; or (iii) 1 of said ring atoms is a nitrogen atom and 1 of said ring atoms is a sulfur atom and the remaining ring atoms are carbon atoms, wherein that mono- or bicyclic aromatic ring system may be unsubstituted or monosubstituted with straight-chain or branched C₁₋₄-alkyl or R^{X1b} or disubstituted with independently from each other straight-chain or branched C₁₋₄-alkyl; preferably, it denotes 1H-indol-6-yl, N-methyl-indol-6-yl (1-methyl-1H-indol-6-yl), 1-methyl-1H-indol-5-yl, 3-methyl-1H-indol-5-yl, 1,3-dimethyl-1H-indol-5-yl, 1-ethyl-1H-indol-6-yl, 1-ethyl-1H-indol-5-yl, 3-methyl-1-benzofuran-5-yl, 3-methyl-1-benzothiophen-5-yl, 1-methyl-1H-indazol-6-yl, 2-amino-1,3-benzothiazol-5-yl, 1-methyl-1H-pyrrolo[2,3-b]pyrdin-6-yl; in particular N-methyl-indol-6-yl, 3-methyl-1-benzofuran-5-yl, 1-methyl-1H-pyrrolo[2,3-b]pyrdin-6-yl;
Ar^{Y1} denotes phenyl;
LA^{Z1} denotes a divalent straight-chain or branched C₁₋₄-alkylene radical, preferably CH₂;
R^{X1a}, R^{X2a} denote independently from each other straight-chain or branched —C₁₋₆-alkyl or —O—C₁₋₆-alkyl, —NH₂, —NHR^{X7a}, —NR^{X7a}R^{X8a} or form together a divalent alkylene chain with 3, 4, 5 chain carbon atoms wherein 1 or 2 of non-adjacent CH₂ groups of the divalent alkylene chain may be replaced independently from each other by —N(H)—, —N(C₁₋₆-alkyl)-, —N(—C(═O)—C₁₋₄-alkyl)-, —O— —wherein that C₁₋₆-alkyl and C₁₋₄-alkyl radicals may be straight-chain or branched—, which divalent alkylene chain may be unsubstituted or mono- or di-substituted with independently from each other straight-chain or branched C₁₋₆-alkyl or ═O (oxo);
R^{X1b} denotes —O-methyl, —NH₂, —C(═O)-methyl;
R^{X7a}, R^{X8a} denote independently from each other straight-chain or branched —C₁₋₄-alkyl.

Still another preferred particular embodiment, PE4c, comprises compounds of formula (I) that embodiment being a combination of particular embodiment PE4 or PE4a or PE4b with one or more of other particular embodiments PE1, PE1a, PE2, PE3, PE3a. An especially preferred particular embodiment, PE4d, is a combination of particular embodiment PE4b with PE1, PE1a, PE2 and PE3 such that it comprises compounds of formula (I) wherein
R¹ denotes Ar^{X1} or Hetar^{X1}; in particular Hetar^{X1};
Ar^{X1} denotes 3-(methylamino)-4-methylphenyl, 3-(dimethylamino)-4-methylphenyl, 3-(dimethylamino)-4-methoxyphenyl, 1-methyl-2,3-dihydro-1H-indol-6-yl (phenyl with R^{X1a} in 3-position and R^{X2a} in 4-position, R^{X1a} and R^{X2a} forming together a —N(CH₃)—CH₂—CH₂-chain), 1-methyl-1,2,3,4-tetrahydroquinolin-7-yl (phenyl with R^{X1a} in 3-position and R^{X2a} in 4-position, R^{X1a} and R^{X2a} forming together a —N(CH₃)—CH₂—CH₂—CH₂— chain), 4-methyl-1,2,3,4-tetrahydroquinoxalin-6-yl (phenyl with with R^{X1a} in 3-position and R^{X2a} in 4-position, R^{X1a} and R^{X2a} forming together a —N(CH₃)—CH₂—CH₂—NH— chain), 5-methyl-2,3,4,5-tetrahydro-1H-1,5-benzodiazepin-7-yl, naphthyl;
Hetar^{X1} denotes 1H-indol-6-yl, N-methyl-indol-6-yl (1-methyl-1H-indol-6-yl), 1-methyl-1H-indol-5-yl, 3-methyl-1H-indol-5-yl, 1,3-dimethyl-1H-indol-5-yl, 1-ethyl-1H-indol-6-yl, 1-ethyl-1H-indol-5-yl, 3-methyl-1-benzofuran-5-yl, 3-methyl-1-benzothiophen-5-yl, 1-methyl-1H-indazol-6-yl, 2-amino-1,3-benzothiazol-5-yl, 1-methyl-1H-pyrrolo[2,3-b]pyrdin-6-yl; in particular N-methyl-1H-indol-6-yl, 3-methyl-1-benzofuran-5-yl, 1-methyl-1H-pyrrolo[2,3-b]pyrdin-6-yl.
R² denotes H;
R³ denotes H;
X denotes N—R⁷;
R⁷ denotes H;
and R⁴, R⁵, R⁶ have the meaning as given above for formula (I), preferably the meaning as given for PE1.

A further particular embodiment of the present invention, PE5, which may optionally be part of any of the above described particular embodiments PE1, PE1a, PE2, PE3, PE3a, PE4, PE4a, PE4b, PE4c and/or PE4d, comprises compounds of formula (I) wherein
R⁵ and R⁶ both denote H, i.e. compounds of formula (IA):

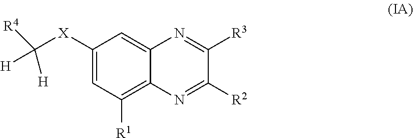

A preferred particular embodiment PE5a of particular embodiment PE5 comprises compounds of formula (I) or (IA) wherein
R⁴ denotes Ar^{X}, Ar^{X}-Hetar^{Y}, Ar^{X}-Hetcyc^{Y}, Hetar^{X}, Hetar^{X}-Hetar^{Y}, Hetar^{X}-Hetcyc^{Y}, Hetcyc^{X}, Hetcyc^{X}-Hetar^{Y}, LA^{Z}-Hetar^{Y}.

It is especially preferred and referred to as PE5b that
R⁴ denotes Ar^{X4}, Ar^{X4}-Hetar^{Y4}, Hetar^{X4}, Hetar^{X4}-Hetar^{Y4}, Hetar^{X4}-Hetcyc^{Y4}, Hetcyc^{X4}, LA^{Z4}-Hetar^{Y4};
Ar^{X4} denotes phenyl which may be unsubstituted or mono- or disubstituted with independently from each other R^{X1c}, R^{X2c};
Hetar^{X4} denotes a mono- or bicyclic aromatic ring system with 5, 6, 8, 9, 10 ring atoms wherein 1, 2, 3 of said ring atoms is/are a hetero atom(s) selected from N, O and/or S and the remaining are carbon atoms, wherein that aromatic ring system may be unsubstituted or mono- or disubstituted with independently from each other R^{X1d}, R^{X2}d;
Hetcyc^{X4} denotes a saturated or partially unsaturated mono-cyclic heterocycle with 3, 4, 5, 6, 7 ring atoms wherein
   (i) 1 ring atom is a heteroatom selected from N, O; or (ii) 1 ring atom is N and 1 ring atom is O; or (iii) 2 ring atoms are N;
   and the remaining ring atoms are carbon atoms,
wherein that heterocycle may be unsubstituted or monosubstituted with R^{X4a};
Hetar^{Y4} denotes a monocyclic aromatic ring system with 5 or 6 ring atoms wherein 1, 2, 3, 4 of said ring atoms are N and the remaining are carbon atoms. wherein that aromatic ring system may be unsubstituted or monosubstituted with R^{Y4a};
Hetcyc^{Y4} denotes a saturated or partially unsaturated mono-cyclic heterocycle with 3, 4, 5, 6, 7 ring atoms wherein
   (i) 1 ring atom is a heteroatom selected from N, O; or (ii) 1 ring atom is N and 1 ring atom is O; or (iii) 2 ring atoms are N;
   and the remaining ring atoms are carbon atoms,
wherein that heterocycle may be unsubstituted or mono-substituted with R^{Y4b};
LA^{Z4} denotes a divalent straight-chain or branched C₁₋₆-alkylene radical;
R^{X1c}, R^{X2c}, R^{X1d}, R^{X2d} denote independently from each other Hal, R^{X9b}, —CN, —NO₂, —SO₂NH₂, —SO₂—R^{X9b}, —NH₂, —OH, —O—R^{X9b}, —C(═O)—NH₂
or
R^{X1d} and R^{X2d} form a divalent alkylene chain with 3 or 4 carbon atoms wherein 1 or 2 of non-adjacent CH₂ groups of the divalent alkylene chain may be replaced independently from each other by —N(H)—, —O— which divalent alkylene chain may be unsubstituted or monosubstituted with ═O (oxo);
R^{X4a} denotes ═O (oxo), straight-chain or branched C₁₋₆-alkyl;
R^{X9b} denotes straight-chain or branched C₁₋₆-alkyl;
R^{Y4a} denotes NH₂, straight-chain or branched C₁₋₆-alkyl
R^{Y4b} denotes straight-chain or branched —C₁₋₆-alkyl, —C(═O)—R^{X9b};
Hal denotes F, Cl, Br, I.

In an especially preferred particular embodiment PE5c compounds of formula (I) or (IA) are comprised wherein
R⁴ denotes pyridin-3-yl-methyl, pyridinyl, oxanyl, thiazol-4-yl, thiazol-5-yl, 1,2-thiazolyl, 1,3-thiazolyl, methylthiazolyl, 3-methyl-1,2-thiazol-5-yl, 5-(1-methyl-1H-pyrazol-4-yl)pyridin-3-yl, 4-benzonitrile, 3-benzonitrile, 5-(1H-imidazol-1-yl)pyridin-3-yl, 5-(2-aminopyrimidin-5-yl)pyridin-3-yl, 5-(1H-pyrazol-4-yl))pyridin-3-yl, 4-(1-methyl-1H-pyrazol-4-yl)pyridin-2-yl, 2-(1-methyl-1H-pyrazol-4-yl)pyridin-4-yl, 1-methyl-1H-imidazol-5-yl, dimethylimidazolyl, 1,2-dimethyl-1H-imidazol-5-yl, triazolyl, 4H-1,2,4-triazol-3-yl, methyltriazolyl, 4-methyl-4H-1,2,4-triazol-3-yl, 1-methyl-1H-1,2,3-triazol-5-yl, 5-methyl-1H-1,2,4-triazol-3-yl, oxazolyl (1,3-oxazolyl), methyloxazolyl, 2-methyl-1,3-oxazol-5-yl, isoxazolyl (1,2-oxazolyl), methyloxadiazolyl, 2-methyl-1,3,4-oxydiazol-5-yl, 4-(1H-1,2,3,4-tetrazol-5-yl)phenyl, 3-(1H-1,2,3,4-tetrazol-5-yl)phenyl, 3-benzamide, 3-aminophenyl, phenyl, furan-2-yl, piperindin-3-yl, morpholin-2-yl, 1H-pyrazol-4-yl, methylpyrazolyl, 1-methyl-1H-pyrazol-5-yl, 1-methyl-1H-pyrazol-4-yl, 2-methansulfonylphenyl, 4-methansulfonylphenyl, 3-methansulfonylphenyl, piperidin-2-yl, pyridazin-3-yl, pyridazin-4-yl, methoxypyridinyl, 4-methoxypyridin-3-yl, 5-bromopyridin-3-yl, 4-bromo-pyridin-2-yl, 2-bromopyridin4-yl, cyanopyridinyl, 4-cyanopyridin-3-yl, 5-(pyrimidin-5-yl)pyridin-3-yl, aminopyridinyl, 5-aminopyridin-3-yl, 4-amino-pyridin-3-yl, 5-(1H-pyrazol-5-yl)pyridin-3-yl, N-acetylpiperazinyl-pyridinyl, 4-(4-acetylpiperazin-1-yl)pyridin-3-yl, acetylmorpholinyl, pyrazolylpyridin-3-yl, imidazopyridinyl, methylpiperazinylpyridinyl, pyrimidinylpyridinyl, methylmorpholinyl, pyrimidinyl, chloropyrimidinyl, aminopyrimidinyl, acetylpiperidinyl, pyridinonyl (hydroxypyridinyl), methylpiperidinyl, hydroxypyridinyl, fluoropyridinyl, methylpyridinyl, methoxypyridinyl, morpholinylpyridinyl; preferably, it denotes pyridin-3-yl, 3-bromopyridin-3-yl, oxan-3-yl, 1,2-thiazol-4-yl, 1,2-thiazol-5-yl, 1,3-thiazol-5-yl, 1-methyl-1H-imidazol-5-yl, 5-(1-methyl-1H-pyrazol-4-yl)pyridin-3-yl, 5-(1H-imidazol-1-yl)pyridin-3-yl, 4H-1,2,4-triazol-3-yl, 1-methyl-1H-1,2,3-triazol-5-yl, 1,2-oxazol-4-yl, 1,3-oxazol-5-yl, 5-(2-aminopyrimidin-5-yl)pyridin-3-yl, 5-(1H-pyrazol-4-yl)pyridin-3-yl, morpholin-2-yl, piperidin-2-yl, 4-(4-acetylpiperazin-1-yl)pyridin-3-yl, 4-methoxypyridin-3-yl, 5-bromopyridin-3-yl, 4-acetylmorpholin-2-yl, methylpyrazolylpyridin-3-yl, 4-(1-methyl-1H-pyrazol-4-yl)-pyridin-3-yl, imidazo[1,2-a]pyridin-6-yl, 4-(4-methylpiperazinyl)pyridin-3-yl, 4-(pyrimidin-5-yl)pyridin-3-yl, 4-methylmorpholin-2-yl, 2-chloro-pyrimidin-5-yl, 2-aminopyrimidin-5-yl, N-acetylpiperidin-2-yl, 1,2-dihydropyridin-2-on-5-yl (2-hydroxypyridin-5-yl), N-methylpiperidin-2-yl, 3-hydroxypyridinyl, 4-fluoropyridin-3-yl, 4-methylpyridin-3-yl, 3-N-morpholinylpyridin-5-yl.

Still another preferred particular embodiment, PE5d, comprises compounds of formula (I) or (IA) that embodiment being a combination of particular embodiment PE5 or PE5a or PE5b or PE5c with one or more of other particular embodiments PE1, PE1a, PE2, PE3, PE3a, PE4, PE4a, PE4b, PE4c, PE4d. An especially preferred particular embodiment, PE5e, is a combination of particular embodiment PE5c with PE1, PE2, PE3, PE4 such that it comprises compounds of formula (I) wherein
R¹ denotes Hetar^{X1};
Hetar^{X1} denotes N-methyl-1H-indol-6-yl, 3-methyl-1-benzofuran-5-yl, 1-methyl-1H-indazol-6-yl, 1-methyl-1H-pyrrolo[2,3-b]pyrdin-6-yl.
R² denotes H;
R³ denotes H;
R⁴ denotes pyridin-3-yl, 3-bromopyridin-3-yl, oxan-3-yl, 1,2-thiazol-4-yl, 1,2-thiazol-5-yl, 1,3-thiazol-5-yl, 5-(1-methyl-1H-pyrazol-4-yl)pyridin-3-yl, 5-(1H-imidazol-1-yl)pyridin-3-yl, 4H-1,2,4-triazol-3-yl, 1-methyl-1H-1,2,3-triazol-5-yl, 1,2-oxazol-4-yl, 1,3-oxazol-5-yl, 5-(2-aminopyrimidin-5-yl)pyridin-3-yl, 5-(1H-pyrazol-4-yl)pyridin-3-yl, morpholin-2-yl, piperidin-2-yl, 4-(4-acetylpiperazin-1-yl)pyridin-3-yl, 4-methoxypyridin-3-yl, 5-bromopyridin-3-yl;
R⁵ and R⁶ both denote H;
X denotes N—R⁷;
R⁷ denotes H.

A further particular embodiment of the present invention, PE6, which may optionally be part of any of the above described particular embodiments PE1, PE1a, PE2, PE3, PE3a, PE4, PE4a, PE4b, PE4c, PE4d, comprises compounds of formula (I) wherein
R⁵ denotes Hetar^{X}, Hetcyc^{X}, LA^{X}, CA^{X};
R⁶ denotes H;
   i.e., compounds of formula (IB)

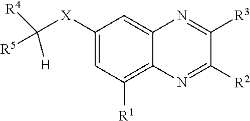

(with R⁵ being Hetar^{X}, Hetcyc^{X} or LA^{X}) (IB)

A preferred particular embodiment PE6a of particular embodiment PE6 comprises compounds of formula (I) or (IB) wherein
R⁵ denotes Hetar^{X5}, Hetcyc^{X5}, LA^{X5}, CA^{X5};
Hetar^{X5} denotes a mono- or bicyclic aromatic ring system with 5, 6, 8, 9, 10 ring atoms wherein 1, 2, 3, 4 of said ring atoms is/are a hetero atom(s) selected from N, O and/or S and the remaining are carbon atoms, wherein that aromatic ring system may be unsubstituted or mono- or disubstituted with independently from each other R^{X1e}, R^{X2e};
Hetcyc^{X5} denotes a saturated monocyclic heterocycle with 3, 4, 5, 6, 7 ring atoms wherein 1 or 2 ring atom(s) is/are heteroatom(s) selected from N and/or O and the remaining ring atoms are carbon atoms, wherein that heterocycle may be unsubstituted or monosubstituted with R^{X4a};
LA^{X5} denotes straight-chain or branched C₁₋₆-alkyl which may be unsubstituted or mono-, di- or trisubstituted with independently from each other Hal or —CN, or monosubstituted with —C(═O)—R^{X9c}, —COOH, —C(═O)—O—R^{X9c}, —C(═O)—NH₂, —C(═O)—NHR^{X7c}, —C(═O)NR^{X7c}R^{X8c};
CA^{X5} denotes a saturated monocyclic carbocycle with 3, 4, 5, 6, 7 carbon atoms which carbocycle may be unsubstituted or monosubstituted with —OH, —NH₂, —NH—C(═O)—R^{X9c};
R^{X1e}, R^{X2e} denote independently from each other Hal, R^{X9c}, —CN, —NO₂, —SO₂NH₂, —SO₂—R^{X9c}, —NH₂, —NHR^{X7c}, —NR^{X7c}R^{X8c}, —OH, —O—R^{X9c}, —C(═O)—NH₂;
R^{X4a} denotes H, LA^{X5a}, Hal, R^{X9c}, —SO₂—R^{X9c}, —CHO, —C(═O)—R^{X9c}, —COOH, —C(═O)—O—R^{X9c}, —C(═O)—NH₂, —C(═O)—NHR^{X7c}, —C(═O)—NR^{X7c}R^{X8c}, oxo (═O);
LA^{X5a} denotes straight-chain or branched —C₁₋₆-alkyl which may be unsubstituted or mono-, di- or trisubstituted with independently from each other Hal or mono- or disubstituted with independently from each other Hal, —CN, oxo, —O—R^{X9c}, —NH₂, —NHR^{X7c}, —NR^{X7c}R^{X8c}, —COOH, —C(═O)—O—R^{X9c}, —C(═O)—NH₂, —C(═O)—NHR^{X7c}, —C(═O)—NR^{X7c}R^{X8c} or —C(═O)—R^{X9c};
R^{X7c}, R^{X8c} denote independently from each other straight-chain or branched —C₁₋₆-alkyl or form together with the nitrogen atom to which they are attached to a 3, 4, 5, 6 or 7 membered heterocycle wherein that heterocycle may not contain any further heteroatom or may contain besides said nitrogen atom one further hetero ring atom selected from N, O and S, wherein, if that further hetero atom is N, that further N may be substituted with H or straight-chain or branched —C₁₋₆-alkyl;
R^{X9c} denote straight-chain or branched C₁₋₆-alkyl or a saturated monocyclic carbocycle with 3, 4, 5, 6, 7 carbon atoms;
Hal denotes F, Cl, Br, I.

An especially preferred particular embodiment, PE6b, of PE6a comprises compounds wherein
R⁵ denotes LA^{X5}—in particular straight-chain C₁₋₆-alkyl—, CA^{X5}, Hetar^{X5} or Hetcyc^{X5};
Hetar^{X5} denotes a substituted or in particular an unsubstituted monocyclic aromatic ring system with 5 or 6 ring atoms wherein 1, 2, 3 or 4—in particular 1 or 2—of said ring atoms is/are a nitrogen atom(s), 0 or 1 of said ring atoms is an oxygen or a sulfur atom and the remaining are carbon atoms, wherein that aromatic ring system may be unsubstituted or monosubstituted with R^{X1e};
Hetcyc^{X5} denotes a saturated monocyclic heterocycle with 3, 4, 5, 6, 7 ring atoms wherein 1 or 2 ring atom(s) is/are heteroatom(s) selected from N and/or O and the remaining ring atoms are carbon atoms, wherein that heterocycle may be unsubstituted or mono-substituted with R^{X4a};
LA^{X5} denotes straight-chain or branched —C₁₋₆-alkyl which may be unsubstituted or monosubstituted with —C(═O)—NH₂, —C(═O)—NHR^{X7c}, —C(═O)—NR^{X7c}R^{X8c};
CA^{X5} denotes a saturated monocyclic carbocycle with 3, 4, 5, 6, 7 carbon atoms which carbocycle may be unsubstituted or monosubstituted with —OH, —NH₂, —NH—C(═O)—R^{X9c};
R^{X1e} denotes R^{X9c};
R^{X4a} denotes H, LA^{X5a}, R^{X9c}, —SO₂—R^{X9c}, —C(═O)—R^{X9c}, —C(═O)—NHR^{X7c}, —C(═O)—NR^{X7c}R^{X8c}, oxo (═O);
LA^{X5a} denotes straight-chain or branched —C₁₋₆-alkyl which may be unsubstituted or monosubstituted with —CN, oxo, —COOH, —C(═O)—NH₂, —C(═O)—NHR^{X7c}, —C(═O)—NR^{X7c}R^{X8c} or —C(═O)—R^{X9c} or disubstituted with oxo and —O—R^{X9c} or —NH₂;
R^{X7c}, R^{X8c} denote independently from each other straight-chain or branched —C₁₋₆-alkyl or form together with the nitrogen atom to which they are attached to a 3, 4, 5, 6 or 7 membered heterocycle wherein that heterocycle may not contain any further heteroatom or may contain besides said nitrogen atom one further hetero ring atom selected from N, O and S, wherein, if that further hetero atom is N, that further N may be substituted with H or straight-chain or branched —C₁₋₆-alkyl;
R^{X9c} denotes straight-chain or branched —C₁₋₆-alkyl or a saturated monocyclic carbocycle with 3, 4, 5, 6, 7 carbon atoms.

A further particular embodiment, PE6c, which may also be part of the particular embodiments PE6, PE6a, PE6b, comprises compounds of formula (I) or (IB) wherein
R⁴ denotes Ar^{X}, Ar^{X}-Hetar^{Y}, Hetar^{X}, Hetar^{X}-Hetar^{Y}, Hetar^{X}-Hetcyc^{Y}, LA^{Z}-Hetcyc^{Y} or Hetcyc^{X}.

A preferred particular embodiment, PE6d, of PE6c comprises compounds of formula (I) or (IB) wherein
R⁴ denotes Ar^{X4}, Ar^{X4}-Hetar^{Y4}, Hetar^{X4}, Hetar^{X4}-Hetar^{Y4}, Hetar^{X4}-Hetcyc^{Y4}, Hetcyc^{X4};
Ar^{X4} denotes phenyl which may be unsubstituted or mono- or disubstituted with independently from each other R^{X1f}, R^{X2f};
Hetar^{X4} denotes a mono- or bicyclic aromatic ring system with 5, 6, 8, 9, 10 ring atoms wherein 1, 2, 3 of said ring atoms is/are a hetero atom(s) selected from N, O and/or S and the remaining are carbon atoms, wherein that aromatic ring system may be unsubstituted or mono- or disubstituted with independently from each other R^{X1g}, R^{X2g};
Hetar^{Y4} denotes a monocyclic aromatic ring system with 5, 6 ring atoms wherein 1, 2, 3, 4 of said ring atoms are N and the remaining are carbon atoms wherein that aromatic ring system may be unsubstituted or monosubstituted with R^{Y4b};
Hetcyc^{X4} denotes a partially unsaturated monocyclic heterocycle with 5, 6, 7, 8 ring atoms wherein 1, 2, 3, 4 ring atom(s) is/are heteroatom(s) selected from N, O and/or S and the remaining ring atoms are carbon atoms, wherein that heterocycle may be unsubstituted or mono- or disubstituted with R^{X4b}, R^{X5b};
Hetcyc^{Y4} denotes a saturated monocyclic heterocycle with 3, 4, 5, 6, 7 ring atoms wherein 1 or 2 ring atom(s) is/are heteroatom(s) selected from N and/or O and the remaining ring atoms are carbon atoms, wherein that heterocycle may be unsubstituted or mono-substituted with R^{Y4b};
R^{X1f}, R^{X2f}, R^{X1g}, R^{X2g} denote independently from each other Hal, R^{X9d}, —CN, —NO₂, —SO₂NH₂, —SO₂—R^{X9d}, —NH₂, —NHR^{X7d}, —NR^{X7d}R^{X8d}, —NH—C(═O)—R^{X9d}, —OH, —O—R^{X9d}, —C(═O)—NH₂;
R^{X4b}, R^{X5b} denote independently from each other oxo (═O), R^{X9d};
R^{Y4b} denotes NH₂, straight-chain or branched C₁₋₆-alkyl;
R^{X7d}, R^{X8d}, R^{X9d} denote independently from each other straight-chain or branched C₁₋₆-alkyl.

In an especially preferred particular embodiment, PE6e, of PE6d
R⁴ denotes pyridinyl, pyrazinyl, pyrimidinyl, methylpyridinyl, 4-methylpyridin-3-yl, methoxypyridinyl, 2-methoxy-pyridin-4-yl, 4-methoxy-pyridin-3-yl, 6-methoxy-pyridin-3-yl, aminopyridinyl, 2-amino-pyridin-4-yl, 6-aminopyridin-3-yl, methylaminopyridinyl, 6-methylaminopyridin-3-yl, methylpiperazinylpyridinyl, 4-(1-methylpiperazin-4-yl)pyridin-3-yl, methylpyrazolylpyridinyl, 4-(1-methyl-1H-pyrazol-4-yl)pyridin-3-yl, 5-(1-methyl-1H-pyrazolyl)pyridinyl, methylimidazolyl, 1-methyl-1H-imidazol-4-yl, 1-methyl-1H-imidazol-5-yl, methyltriazolyl, phenyl, 3-methoxyphenyl, 4-methoxyphenyl, 3-(SO₂NH₂)-phenyl (3-aminosulfonylphenyl); preferably pyridin-3-yl, pyridin-4-yl, pyrazin-2-yl, 5-(1-methyl-1H-pyrazol-4-yl)pyridin-3-yl, methyl-dihydropyridinonyl, 1-methyl-1,2-dihydropyridin-2-on-5-yl;
R⁵ denotes methyl, —CH₂—C(═O)—N(CH₃)₂, hydroxycyclohex-4-yl, aminocyclohex-4-yl, CH₃—C(═O)—NH—cyclohex-4-yl, acetylazetidinyl, 1-acetylazetidin-3-yl, piperidinyl, methylpiperidinyl, acetylpiperidinyl, N-cyanomethylpiperidinyl, N—(CH₃CH₂C(═O)-)piperidinyl, N—((CH₃)₂CH—C(═O)-)piperidinyl, 1-(2-methoxy-ethan-1-onyl)-piperdin-4-yl (1-(CH₃O—CH₂—C(═O)-)piperidin-4-yl), 1-(butan-1-on-1-yl)piperidin-4-yl, 1-(propan-2-on-1-yl)piperidin-4-yl (1-(CH₃—C(═O)—CH₂-)piperidin-4-yl, 1-(HOOC—CH₂-)piperidin-4-yl, 1-(CH₃—NH—C(═O)-)piperidin-4-yl, 1-((CH₃)₂N—C(═O)-)piperidin-4-yl, 1-(NH₂—C(═O)—CH₂)piperidin-4-yl, 1-(CH₃—NH—C(═O)—CH₂)piperidin-4-yl, 1-((CH₃)₂N—C(═O)—CH₂)piperidin-4-yl, 1-((CH₃CH₂)₂N—C(═O)—CH₂)piperidin-4-yl, 1-cyclopropanecarbonylpiperidin-4-yl, 1-(NH₂—CH₂—C(═O)-)piperidin-4-yl, 1-(CH₃—CH(—NH₂)—C(═O)-)piperidin-4-yl, 1-methanesulfonylpiperidin-4-yl, dihydropyridinonyl, 1-(NH₂—CH₂CH₂—C(═O)-)piperidin-4-yl, 1,2-dihydropyridin-2-on-5-yl (6-hydroxypyridin-3-yl), 1,2-dihydropyridin-2-on-4-yl (2-hydroxypyridin-4-yl), oxanyl, imidazolyl, methylimidazolyl, 1-methyl-1H-imidazol-5-yl, pyrazolyl, methylpyrazolyl, 1-methyl-1H-pyrazol-5-yl, triazolyl, methyltriazolyl, or pyridinyl; preferably methyl, piperidin-4-yl, N-acetylpipridin-4-yl, N-methylpiperidin-4-yl, 1-methyl-1H-1,2,3-triazol-5-yl, oxan-4-yl, 1-methyl-1H-1,2,3-triazol-5-yl, tetrazolyl, methyltetrazolyl, 1-methyl-1H-1,2,3,4-tetrazol-5-yl or pyridin-3-yl.

Still another preferred particular embodiment, PE6f, comprises compounds of formula (I) or (IB) that embodiment being a combination of particular embodiment PE6 or PE6a or PE6b or PE6c or PE6d or PE6e with one or more of other particular embodiments PE1, PE1a, PE2, PE3, PE3a, PE4, PE4a, PE4b, PE4c, PE4d. An especially preferred particular embodiment, PE6g, is a combination of particular embodiment PE6e with PE1, PE1a, PE2, PE3, PE4 such that it comprises compounds of formula (I) wherein
R¹ denotes Hetar^{X1};
Hetar^{X1} denotes N-methyl-1H-indol-6-yl, 3-methyl-1-benzofuran-5-yl, 3-methyl-1-benzothiophen-5-yl, 2-amino-1,3-benzothiazol-5-yl, 1-methyl-1H-pyrrolo[2,3-b]pyrdin-6-yl;
R² denotes H;
R³ denotes H;
R⁴ denotes pyridin-3-yl, pyridin-4-yl, pyrazin-2-yl, 4-methylpyridin-3-yl, 2-methoxy-pyridin-4-yl, 6-methoxy-pyridin-3-yl, 2-amino-pyridin-4-yl, 6-aminopyridin-3-yl, 4-(1-methylpiperazin-4-yl)pyridin-3-yl, 4-(1-methyl-1H-pyrazol-4-yl)pyridin-3-yl, 5-(1-methyl-1H-pyrazol-4-yl)pyridin-3-yl, 1-methyl-1H-imidazol-5-yl, 1-methyl-1H-1,2,3-triazol-5-yl;
R⁵ denotes methyl, aminocyclohex-4-yl, CH₃—C(═O)—NH—cyclohex-4-yl, piperidin-4-yl, 1-acetylpiperidin-3-yl, N-acetylpiperidin-4-yl, N-methylpiperidin-4-yl, 1-cyanomethylpiperidin-4-yl, 1-(CH₃CH₂C(═O)-)piperidin-4-yl (1-(ethylcarbonyl)piperidin-4-yl), 1-((CH₃)₂CH—C(═O)-)piperidin-4-yl, 1-(2-methoxy-ethan-1-onyl)-piperdin-4-yl (1-(CH₃O—CH₂—C(═O)-)piperidin-4-yl), 1-(butan-1-on-1-yl)piperidin-4-yl, 1-(propan-2-on-1-yl)piperidin-4-yl, 1-cyclopropanecarbonylpiperidin-4-yl, 1-(CH₃—NH—C(═O)-)piperidin-4-yl, 1-((CH₃)₂N—C(═O)-)piperidin-4-yl, 1-(NH₂—C(═O)—CH₂)piperidin-4-yl, 1-(CH₃—NH—C(═O)—CH₂)piperidin-4-yl, 1-((CH₃)₂N—C(═O)—CH₂)piperidin-4-yl, 1,2-dihydropyridin-2-on-5-yl (6-hydroxypyridin-3-yl), 1,2-dihydropyridin-2-on-4-yl, 1-methyl-1H-imidazol-5-yl, 1-methyl-1H-1,2,3-triazol-5-yl, oxan-4-yl or pyridin-3-yl;
R⁶ denotes H;
X denotes N—R⁷;
R⁷ denotes H.

A further particular embodiment of the present invention, PE7, which may optionally be part of any of the above described particular embodiments PE1, PE1a, PE2, PE3, PE3a, PE4, PE4a, PE4b, PE4c, PE4d, comprises compounds of formula (I) wherein
R⁵, R⁶ both denote independently from each other Ar^{X}, Hetar^{X}, Hetcyc^{X}, LA^{X} or
R⁵ and R⁶ form together with the carbon atom to which they are attached to a saturated ring system D which ring system D is mono- or bicyclic and has 3, 4, 5, 6, 7, 8, 9, 10 ring atoms and may contain no hetero ring atom or 1, 2, 3 hetero ring atom(s) independently from each other selected from N, O and/or S that ring system D may be unsubstituted or mono-, di- or trisubstituted with independently from each other R^{D1}, R^{D2}, R^{D3};
R^{D1}, R^{D2}, R^{D3} are as defined above for compounds of formula (I) or in claim 1.

A preferred particular embodiment PE7a of particular embodiment PE7 comprises compounds of formula (I) wherein
R⁵ denotes LA^{X5};
R⁶ denotes LA^{X6};
or
R⁵ and R⁶ form together with the carbon atom to which they are attached to a saturated ring system D which ring system D is mono- or bicyclic and has 3, 4, 5, 6, 7, ring atoms and may contain no hetero ring atom or 1 hetero ring atom selected from N, O and/or S that ring system D may be unsubstituted or monosubstituted with straight-chain or branched —C₁₋₆-alkyl;
LA^{X5}, LA^{X6} denote independently from each other straight-chain or branched —C₁₋₆-alkyl.

In an especially preferred particular embodiment, PE7b, of PE7a R⁵ and R⁶ both have the same meaning, preferably straight-chain or branched —C₁₋₆-alkyl, more preferably methyl.

In still another especially preferred particular embodiment, PE7c, of PE7a R⁵ and R⁶ form together with the carbon atom to which they are attached to a saturated ring system D which ring system D is selected from

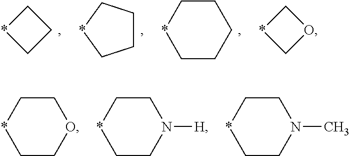

wherein the asterisk “*” denotes the carbon atom to which R⁵ and R⁶ are attached to.

A further particular embodiment of the present invention, PE7c, which may optionally be part of any of the above described particular embodiments PE1, PE1a, PE2, PE3, PE3a, PE4, PE4a, PE4b, PE4c, PE4d, comprises compounds of formula (I) wherein
R¹ denotes Hetar^{X1};
Hetar^{X1} denotes N-methyl-1H-indol-6-yl, 3-methyl-1-benzofuran-5-yl, 1-methyl-1H-pyrrolo[2,3-b]pyrdin-6-yl.
R² denotes H;
R³ denotes H;
R⁴ denotes pyridinyl, pyridin-3-yl, pyridin-4-yl, 5-(1-methyl-1H-pyrazol-4-yl)pyridin-3-yl, 5-(1H-imidazol-1-yl)pyridin-3-yl, 5-(2-aminopyrimidin-5-yl)pyridin-3-yl, 5-(1H-pyrazol-4-yl))pyridin-3-yl, 5-bromopyridin-3-yl, 5-(pyrimidin-5-yl)pyridin-3-yl, 5-aminopyridin-3-yl, 5-(1H-pyrazol-5-yl)pyridin-3-yl;
R⁵ and R⁶ both denote methyl;
or
R⁵ and R⁶ form together with the carbon atom to which they are attached to a saturated ring system D which ring system D is selected from

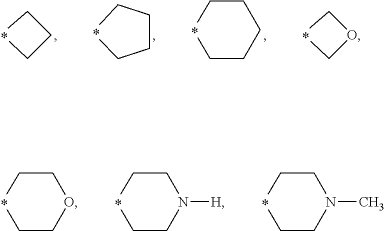

wherein the asterisk “*” denotes the carbon atom to which R⁵ and R⁶ are attached to;
X denotes N—R⁷;
R⁷ denotes H.

A further particular embodiment of the present invention, PE8, which may optionally be part of any of the above described particular embodiments PE1, PE1a, PE2, PE3, PE3a, PE4, PE4a, PE4b, PE4c, PE4d, comprises compounds of formula (I) wherein
R⁴ and R⁵ form together with the carbon atom to which they are attached to a saturated or partially unsaturated ring system A which ring system A is mono- or bicyclic and has 3, 4, 5, 6, 7, 8, 9, 10 ring atoms and may contain no hetero ring atom or 1, 2, 3 hetero ring atom(s) independently from each other selected from N, O and/or S that ring system A may be unsubstituted or mono-, di- or trisubstituted with independently from each other R^{A1}, R^{A2}, R^{A3};
R^{A1}, R^{A2}, R^{A3} are as defined in claim 1 or for the compound of formula (I) hereinabove.

In a preferred particular embodiment, PE8a, of PE8
R⁴ and R⁵ form together with the carbon atom to which they are attached to a saturated or partially unsaturated ring system A which ring system A is mono- or bicyclic and has 4, 5, 6, 7, 8, 9, 10 ring atoms and may contain no hetero ring atom or 1, 2, 3 hetero ring atom(s) independently from each other selected from N, O and/or S that ring system A may be unsubstituted or mono-, disubstituted with independently from each other R^{A1a}, R^{A2a};
R^{A1a}, R^{A2}a denote independently from each other LA^{XA}, —C(═O)—R^{X9A}, oxo (═O), —NH—C(═O)—R^{X9A}, —SO₂—R^{X9A}, phenyl, pyridinyl, methylpyridinyl, pyrimidinyl, hydroxypyrimidinyl, methylpyrimidinyl, pyrazinyl, benzodiazolyl or form together with one carbon atom of ring system A to which they both are attached to a saturated ring system E which ring system E is mono-cyclic and has 3, 4, 5, 6, 7 ring atoms and may contain no hetero atom or 1 hetero atom selected from N and O, that ring system E may be unsubstituted or mono- or disubstituted with independently from each other R^{E1a}, R^{E1b};
LA^{XA}, R^{E1a}, R^{E1b} denote independently from each other straight-chain or branched C₁₋₆-alkyl
R^{X9A} denotes straight-chain or branched —C₁₋₆-alkyl, which may be unsubstituted or monosubstituted with —NH₂, a saturated monocyclic carbocycle with 3, 4, 5, 6, 7 carbon atoms, phenyl or pyridinyl.

It is an especially preferred particular embodiment PE8b of PE8 or PE8a in which
R⁴ and R⁵ form together with the carbon atom to which they are attached to
   (i) a saturated or partially unsaturated monocyclic ring system A with 4, 5, 6 or 7 ring atoms which may contain no hetero ring atom or 1 hetero ring atom selected from N and O that ring system A may be unsubstituted or mono-, disubstituted with independently from each other R^{A1a}, R^{A2a}, or
   (ii) a saturated or partially unsaturated bicyclic ring system A with 9 or 10 ring atoms which may contain no hetero ring atom or 1 hetero ring atom selected from N and O that ring system A may be unsubstituted or mono-, disubstituted with independently from each other R^{A1a}, R^{A2a};
R^{A1a}, R^{A2a} denote independently from each other methyl, —C(═O)-methyl, —C(═O)-ethyl, —C(═O)—CH(CH₃)₂, —C(═O)-(cyclo-C₃H₅), —C(═O)-phenyl, —C(═O)-pyridinyl, —C(═O)—CH₂NH₂, oxo (═O), —NH—C(═O)-methyl, —SO₂-methyl, phenyl, pyridin-2-yl, pyridin-3-yl, 3-methylpyridin-2-yl, pyrimidin-2-yl, pyrimidin-4-yl, pyrimidin-5-yl, 2-hydroxypyrimidin-4-yl, 2-methylpyrimidin-4-yl, pyrazin-2-yl, 1H-1,3-benzodiazol-2-yl or form together with one carbon atom of ring system A to which they both are attached to a saturated ring

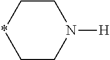 [SMILES: *1CCNCC1]

wherein the asterisk “*” denotes the carbon atom to which R^{A1a} and R^{A2a} are attached to.

It is especially preferred and referred to a PE8c that
R⁴ and R⁵ form together with the carbon atom to which they are attached to oxanyl, dimethyloxanyl, tetrahydronaphthalenyl, tetrahydroquinolinyl, N-acetyltetrahydroquinolinyl, dihydrobenzopyranyl, azetidinyl, N-acetylazetidinyl, pyrrolidinyl, N-methylpyrrolidinyl, N-phenylpyrrolidinyl, N-acetylpyrrolidinyl, N-ethylcarbonylpyrrolidinyl, N—((CH₃)₂—CH—C(═O)-)pyrrolidinyl, N-cyclopropanecarbonylpyrrolidinyl, N-benzoylpyrrolidinyl, N-(pyridinylcarbonyl)pyrrolidinyl, N-(aminomethylcarbonyl)-pyrrolidinyl, N-methanesulfonylpyrrolidinyl, N-(pyridinyl)-pyrrolidinyl, N-(methylpyridinyl)pyrrolidinyl, N-(pyrimidinyl)-pyrrolidinyl, N-(hydroxypyrimidinyl)pyrrolidinyl, N-(methyl-pyrimidinyl)pyrrolidinyl, N-(pyranzinyl)pyrrolidinyl, piperidinyl, N-acetylpiperidinyl, N-(pyrimidinyl)piperidinyl, N-(benzodiazolyl)-pyrrolidinyl, azepanyl, N-acetylazepanyl, N-cyclopropanecarbonylazepanyl, 7-azaspiro[3.5]nonan-1-yl, (CH₃—C(═O)—NH—)cyclohexyl, cyclohexanonyl, piperidinonyl, 2H,3H,4H-pyrano[3,2-b]pyridin-4-yl, 5,6,7,8-tetrahydroquinoxalin-5-yl; preferably, they form oxan-4-yl, 2,3-dimethyloxan-4-yl, 1,2,3,4-tetrahydronaphthalen-1-yl, 5,6,7,8-tetrahydroquinolin-5-yl, 5,6,7,8-tetrahydroquinolin-8-yl, N-acetyl-1,2,3,4-tetrahydroquinolin-4-yl, 3,4-dihydro-2H-1-benzopyran-4-yl, cyclohexan-4-onyl, 2H,3H,4H-pyrano[3,2-b]pyridin-4-yl, 5,6,7,8-tetrahydroquinoxalin-5-yl, 1-acetylazetidin-3-yl, pyrrolidin-3-yl, 1-methylpyrroldin-3-yl, 1-phenylpyrrolidin-3-yl, 1-acetylpyrrolidin-3-yl, 1-(ethylcarbonyl)-pyrrolidin-3-yl, 1-((CH₃)₂—CH—C(═O)-)pyrrolidin-3-yl, 1-cyclopropanecarbonylpyrrolidin-3-yl, 1-benzoylpyrrolidin-3-yl, 1-(pyridin-2-ylcarbonyl)pyrrolidin-3-yl, 1-(aminomethylcarbonyl)-pyrrolidin-3-yl, 1-methanesulfonylpyrrolidin-3-yl, 1-(pyridin-2-yl)pyrrolidin-3-yl, 1-(pyridin-3-yl)pyrrolidin-3-yl, 1-(3-methyl-pyridin-2-yl)pyrrolidin-3-yl, 1-(pyrimidin-2-yl)pyrroldin-3-yl, 1-(pyrimidin-4-yl)pyrroldin-3-yl, 1-(pyrimidin-5-yl)pyrroldin-3-yl, 1-(2-hydroxypyrimidin-4-yl)pyrrolidin-3-yl, 1-(2-methylpyrimidin-4-yl)pyrrolidin-3-yl, 1-(pyranzin-2-yl)pyrrolidin-3-yl, 1-(1H-1,3-benzodiazol-2-yl)pyrrolidin-3-yl, 1-acetylpiperidin-3-yl, 1-acetylpiperidin-4-yl, 1-(pyrimidin-2-yl)piperidin-4-yl, 1-acetylazepan-4-yl, 1-(cyclopropanecarbonyl)azepan-4-yl, 1-(CH₃—C(═O)—NH-)cyclohex-4-yl.

Another particular embodiment, PE8d, of PE8, which may also optionally be part of any of particular embodiments of PE8a, PE8b, PE8c, comprises compounds of formula (I) wherein
R⁶ denotes H.

Still another preferred particular embodiment, PE8e, comprises compounds of formula (I) that embodiment being a combination of particular embodiment PE8 or PE8a or PE8b or PE8c or PE8d with one or more of other particular embodiments PE1, PE1a, PE2, PE3, PE3a, PE4, PE4a, PE4b, PE4c, PE4d. An especially preferred particular embodiment, PE8f, is a combination of particular embodiment PE8d with PE1, PE1a, PE2, PE3, PE4 such that it comprises compounds of formula (I) wherein
R¹ denotes Ar^{X1} or Hetar^{X1};
Ar^{X1} denotes 3-(methylamino)-4-methylphenyl, 3-(dimethylamino)-4-methylphenyl, 3-(dimethylamino)-4-methoxyphenyl, naphthyl, 1-methyl-2,3-dihydro-1H-indol-6-yl (i.e., phenyl with substituent R^{X1a} in 3-position and substituent R^{X2a} in 4-position, wherein R^{X1a} and R^{X2a} are forming together a —N(CH₃)—CH₂—CH₂— chain with the —N(CH₃)-terminus of that chain replacing R^{X1a} and the CH₂-terminus of that chain replacing the R^{X2a} substituent), 4-methyl-1,2,3,4-tetrahydroquinoxalin-6-yl (i.e., phenyl with with substituent R^{X1a} in 3-position and substituent R^{X2a} in 4-position, wherein R^{X1a} and R^{X2a} are forming together a —N(CH₃)—CH₂—CH₂—NH— chain with the —N(CH₃)-terminus of that chain replacing R^{X1a} and the CH₂-terminus of that chain replacing the R^{X2a} substituent), 5-methyl-2,3,4,5-tetrahydro-1H-1,5-benzodiazepin-7-yl (i.e., phenyl with with substituent R^{X1a} in 3-position and substituent R^{X2a} in 4-position, wherein R^{X1a} and R^{X2a} are forming together a —N(CH₃)—CH₂—CH₂—CH₂—NH— chain with the —N(CH₃)-terminus of that chain replacing R^{X1a} and the CH₂-terminus of that chain replacing the R^{X2a} substituent);
Hetar^{X1} denotes N-methyl-1H-indol-6-yl, 1-methyl-1H-indol-5-yl, 1-ethyl-1H-indol-6-yl, 1-ethyl-1H-indol-5-yl, 3-methyl-1H-indol-5-yl, 1,3-dimethyl-1H-indol-5-yl, 3-methyl-1-benzofuran-5-yl, 3-methyl-1-benzothiophen-5-yl, 1-methyl-1H-indazol-6-yl, 1-methyl-1H-pyrrolo[2,3-b]pyrdin-6-yl.
R² denotes H;
R³ denotes H;
R⁴ and R⁵ form together with the carbon atom to which they are attached to oxan-4-yl, 2,3-dimethyloxan-4-yl, 1,2,3,4-tetrahydronaphthalen-1-yl, 5,6,7,8-tetrahydroquinolin-5-yl, 5,6,7,8-tetrahydroquinolin-8-yl, N-acetyl-1,2,3,4-tetrahydroquinolin-4-yl, 3,4-dihydro-2H-1-benzopyran-4-yl, cyclohexan-4-onyl, 1-acetylazetidin-3-yl, pyrrolidin-3-yl, 1-methylpyrroldin-3-yl, 1-phenylpyrrolidin-3-yl, 1-acetylpyrrolidin-3-yl, 1-(ethylcarbonyl)pyrrolidin-3-yl, 1-((CH₃)₂—CH—C(═O)-)pyrrolidin-3-yl, 1-cyclopropanecarbonylpyrrolidin-3-yl, 1-benzoylpyrrolidin-3-yl, 1-(pyridin-2-ylcarbonyl)pyrrolidin-3-yl, 1-(aminomethylcarbonyl)pyrrolidin-3-yl, 1-methanesulfonylpyrrolidin-3-yl, 1-(pyridin-2-yl)pyrrolidin-3-yl, 1-(pyridin-3-yl)pyrrolidin-3-yl, 1-(3-methylpyridin-2-yl)pyrrolidin-3-yl, 1-(pyrimidin-2-yl)pyrroldin-3-yl, 1-(pyrimidin-4-yl)pyrroldin-3-yl, 1-(pyrimidin-5-yl)pyrroldin-3-yl, 1-(2-hydroxypyrimidin-4-yl)pyrrolidin-3-yl, 1-(2-methylpyrimidin-4-yl)pyrrolidin-3-yl, 1-(pyranzin-2-yl)pyrrolidin-3-yl, 1-(1H-1,3-benzodiazol-2-yl)pyrrolidin-3-yl, 1-acetylpiperidin-3-yl, 1-acetylpiperidin-4-yl, 1-(pyrimidin-2-yl)piperidin-4-yl, 1-acetylazepan-4-yl, 1-(cyclopropanecarbonyl)azepan-4-yl, 1-(CH₃—C(═O)—NH—)cyclohex-4-yl;
R⁶ denotes H;
X denotes N—R⁷;
R⁷ denotes H.

It is still another particular embodiment, PE9, of the present invention, that comprises a compound selected from the following group, N-oxides thereof and physiologically acceptable salts either of the compound or any of its N-oxides, the group consisting of:
8-(1-methyl-1H-indol-6-yl)-N-[(1R)-1,2,3,4-tetrahydronaphthalen-1-yl]quinoxalin-6-amine
8-(1-methyl-1H-indol-6-yl)-N-[1-(pyridin-3-yl)ethyl]quinoxalin-6-amine
8-(1-methyl-1H-indol-6-yl)-N-[2-(pyridin-3-yl)ethyl]quinoxalin-6-amine
8-(1-methyl-1H-indol-6-yl)-N-[1-(pyridin-4-yl)ethyl]quinoxalin-6-amine
8-(1-methyl-1H-indol-6-yl)-N-[1-(pyridin-2-yl)ethyl]quinoxalin-6-amine
N-[(1 S)-1-(3-methoxyphenyl)ethyl]-8-(1-methyl-1H-indol-6-yl)quinoxalin-6-amine
2-methoxy-4-(7-{[(1R)-1,2,3,4-tetrahydronaphthalen-1-yl]amino}quinoxalin-5-yl)benzonitrile
8-(1-methyl-1H-1,3-benzodiazol-6-yl)-N-[(1R)-1,2,3,4-tetrahydronaphthalen-1-yl]quinoxalin-6-amine
8-chloro-N-[(1R)-1,2,3,4-tetrahydronaphthalen-1-yl]quinoxalin-6-amine
8-(1-methyl-1H-indol-6-yl)-N-(pyridin-3-ylmethyl)quinoxalin-6-amine
N-[(1R)-1-(3-methoxyphenyl)ethyl]-8-(1-methyl-1H-indol-6-yl)quinoxalin-6-amine
8-(4-amino-3-methoxyphenyl)-N-[(1R)-1,2,3,4-tetrahydronaphthalen-1-yl]quinoxalin-6-amine
8-(5-amino-6-methylpyridin-3-yl)-N-[(1R)-1,2,3,4-tetrahydronaphthalen-1-yl]quinoxalin-6-amine
N-(3,4-dihydro-2H-1-benzopyran-4-yl)-8-(1-methyl-1H-indol-6-yl)quinoxalin-6-amine
N-[1-(4-methoxyphenyl)ethyl]-8-(1-methyl-1H-indol-6-yl)quinoxalin-6-amine
8-(1-methyl-1H-indol-6-yl)-N-(5,6,7,8-tetrahydroisoquinolin-8-yl)quinoxalin-6-amine
8-(2,3-dihydro-1,4-benzodioxin-6-yl)-N-[(1R)-1,2,3,4-tetrahydronaphthalen-1-yl]quinoxalin-6-amine
2-methoxy-4-(7-{[(1R)-1,2,3,4-tetrahydronaphthalen-1-yl]amino}quinoxalin-5-yl)benzamide
8-(1-methyl-1H-indol-6-yl)-N-(5,6,7,8-tetrahydroquinolin-5-yl)quinoxalin-6-amine
8-(1,3-dimethyl-1H-pyrazol-4-yl)-N-[(1R)-1,2,3,4-tetrahydronaphthalen-1-yl]quinoxalin-6-amine
2-{[8-(1-methyl-1H-indol-6-yl)quinoxalin-6-yl]amino}-1-(pyrrolidin-1-yl)propan-1-one
N-(2,2-dimethyloxan-4-yl)-8-(1-methyl-1H-indol-6-yl)quinoxalin-6-amine
8-(1-methyl-1H-indol-6-yl)-N-(oxan-3-ylmethyl)quinoxalin-6-amine
8-(3-amino-4-methoxyphenyl)-N-[(1R)-1,2,3,4-tetrahydronaphthalen-1-yl]quinoxalin-6-amine
8-(4-methoxy-3-nitrophenyl)-N-[(1R)-1,2,3,4-tetrahydronaphthalen-1-yl]quinoxalin-6-amine
8-chloro-N-[1-(pyridin-3-yl)ethyl]quinoxalin-6-amine
8-(1-methyl-1H-indol-6-yl)-N-(1,3-thiazol-4-ylmethyl)quinoxalin-6-amine
3-(1-{[8-(1-methyl-1H-indol-6-yl)quinoxalin-6-yl]amino}ethyl)benzene-1-sulfonamide
1-methyl-6-(7-{[(1R)-1,2,3,4-tetrahydronaphthalen-1-yl]amino}quinoxalin-5-yl)-1H,6H,7H-pyrrolo[2,3-c]pyridin-7-one
N-(furan-2-ylmethyl)-8-(1-methyl-1H-indol-6-yl)quinoxalin-6-amine
1-(4-{[8-(1-methyl-1H-indol-6-yl)quinoxalin-6-yl]amino}-1,2,3,4-tetrahydroquinolin-1-yl)ethan-1-one
N-benzyl-8-(1-methyl-1H-indol-6-yl)quinoxalin-6-amine
2-methyl-8-(1-methyl-1H-indol-6-yl)-N-[1-(pyridin-3-yl)ethyl]quinoxalin-6-amine
3-methyl-8-(1-methyl-1H-indol-6-yl)-N-[1-(pyridin-3-yl)ethyl]quinoxalin-6-amine
8-(1-methyl-1H-indol-6-yl)-N-[(1R)-1-(pyridin-3-yl)ethyl]quinoxalin-6-amine
8-(1-methyl-1H-indol-6-yl)-N-[(1 S)-1-(pyridin-3-yl)ethyl]quinoxalin-6-amine
8-(1-methyl-1H-indol-6-yl)-N-[1-(pyrazin-2-yl)ethyl]quinoxalin-6-amine
8-(1-methyl-1H-indol-6-yl)quinoxalin-6-ol
8-(1-methyl-1H-indol-6-yl)-N-(piperidin-3-yl)quinoxalin-6-amine
8-(1-methyl-1H-indol-6-yl)quinoxalin-6-amine
8-(1-methyl-1H-indol-6-yl)-N-[1-(pyrimidin-5-yl)ethyl]quinoxalin-6-amine
8-(1H-indazol-6-yl)-N-[1-(pyridin-3-yl)ethyl]quinoxalin-6-amine
5-(1-methyl-1H-indol-6-yl)-7-(pyridin-3-ylmethoxy)quinoxaline
8-{1-methyl-1H-pyrrolo[3,2-b]pyridin-6-yl}-N-[1-(pyridin-3-yl)ethyl]quinoxalin-6-amine
8-(1H-indol-6-yl)-N-[1-(pyridin-3-yl)ethyl]quinoxalin-6-amine
8-(1-methyl-1H-indol-6-yl)-6-{[1-(pyridin-3-yl)ethyl]amino}quinoxalin-2-ol
5-(1-methyl-1H-indol-6-yl)-7-{[1-(pyridin-3-yl)ethyl]amino}quinoxalin-2-ol
N-[bis(pyridin-3-yl)methyl]-8-(1-methyl-1H-indol-6-yl)quinoxalin-6-amine
N-[bis(pyridin-3-yl)methyl]-8-chloroquinoxalin-6-amine
8-{1-methyl-1H-pyrrolo[2,3-b]pyridin-6-yl}-N-[1-(pyridin-3-yl)ethyl]quinoxalin-6-amine
2,2,2-trifluoro-N-[8-(1-methyl-1H-indol-6-yl)quinoxalin-6-yl]-N-(piperidin-4-yl)acetamide
8-[1-(2-methoxyethyl)-1H-indol-6-yl]-N-[1-(pyridin-3-yl)ethyl]quinoxalin-6-amine
N-[(4-methanesulfonylphenyl)methyl]-8-(1-methyl-1H-indol-6-yl)quinoxalin-6-amine
8-(1-methyl-1H-indol-6-yl)-N-(pyridazin-3-ylmethyl)quinoxalin-6-amine
N-[(3-methanesulfonylphenyl)methyl]-8-(1-methyl-1H-indol-6-yl)quinoxalin-6-amine
N-[(2-methanesulfonylphenyl)methyl]-8-(1-methyl-1H-indol-6-yl)quinoxalin-6-amine
8-(1-methyl-1H-indol-6-yl)-N-(piperidin-2-ylmethyl)quinoxalin-6-amine
8-(1-methyl-1H-indol-6-yl)-N-(piperidin-3-ylmethyl)quinoxalin-6-amine
5-(7-{[1-(pyridin-3-yl)ethyl]amino}quinoxalin-5-yl)-2,3-dihydro-1H-isoindol-1-one
8-(1-methyl-1H-indol-6-yl)-N-(morpholin-2-ylmethyl)quinoxalin-6-amine
8-(1-methyl-1H-indol-6-yl)-N-(1H-pyrazol-4-ylmethyl)quinoxalin-6-amine
8-(1,3-benzothiazol-6-yl)-N-[1-(pyridin-3-yl)ethyl]quinoxalin-6-amine
3-{[8-(1-methyl-1H-indol-6-yl)quinoxalin-6-yl]amino}-3-(pyridin-3-yl)prop-2-enoic acid
8-[3-(3-aminoazetidin-1-yl)phenyl]-N-[1-(pyridin-3-yl)ethyl]quinoxalin-6-amine
1-[6-(7-{[1-(pyridin-3-yl)ethyl]amino}quinoxalin-5-yl)-2,3-dihydro-1H-indol-1-yl]ethan-1-one
8-{octahydrocyclopenta[c]pyrrol-2-yl}-N-[1-(pyridin-3-yl)ethyl]quinoxalin-6-amine
8-(1-methyl-1H-indol-6-yl)-N-(oxan-4-yl)quinoxalin-6-amine
3-{[8-(1-methyl-1H-indol-6-yl)quinoxalin-6-yl]amino}-3-(pyridin-3-yl)propanoic acid
6-(7-{[1-(pyridin-3-yl)ethyl]amino}quinoxalin-5-yl)-4H-chromen-4-one
8-(1-methyl-1H-indol-6-yl)-N-{[5-(1-methyl-1H-pyrazol-4-yl)pyridin-3-yl]methyl}quinoxalin-6-amine
4-({[8-(1-methyl-1H-indol-6-yl)quinoxalin-6-yl]amino}methyl)benzonitrile
3-({[8-(1-methyl-1H-indol-6-yl)quinoxalin-6-yl]amino}methyl)benzonitrile
N-{[5-(1H-imidazol-1-yl)pyridin-3-yl]methyl}-8-(1-methyl-1H-indol-6-yl)quinoxalin-6-amine
N-{[5-(2-aminopyrimidin-5-yl)pyridin-3-yl]methyl}-8-(1-methyl-1H-indol-6-yl)quinoxalin-6-amine
8-(1-methyl-1H-indol-6-yl)-N-[(4-nitrophenyl)methyl]quinoxalin-6-amine
N-[(4-aminophenyl)methyl]-8-(1-methyl-1H-indol-6-yl)quinoxalin-6-amine
N-[1-(6-methoxypyridin-3-yl)ethyl]-8-(1-methyl-1H-indol-6-yl)quinoxalin-6-amine
8-(1-methyl-1H-indol-6-yl)-N-[(3-nitrophenyl)methyl]quinoxalin-6-amine
N-[(3-aminophenyl)methyl]-8-(1-methyl-1H-indol-6-yl)quinoxalin-6-amine
4-{[8-(1-methyl-1H-indol-6-yl)quinoxalin-6-yl]amino}cyclohexan-1-one
5-{[8-(1-methyl-1H-indol-6-yl)quinoxalin-6-yl]amino}piperidin-2-one
8-(1-methyl-1H-indol-6-yl)-N-[2-(pyridin-3-yl)propan-2-yl]quinoxalin-6-amine
8-(1-methyl-1H-indol-5-yl)-N-[1-(pyridin-3-yl)ethyl]quinoxalin-6-amine
3-({[8-(1-methyl-1H-indol-6-yl)quinoxalin-6-yl]amino}methyl)benzamide
8-(1-methyl-1H-indol-6-yl)-N-{[3-(1H-1,2,3,4-tetrazol-5-yl)phenyl]methyl}quinoxalin-6-amine
N-[(2-methoxypyridin-3-yl)methyl]-8-(1-methyl-1H-indol-6-yl)quinoxalin-6-amine
3-({[8-(1-methyl-1H-indol-6-yl)quinoxalin-6-yl]amino}methyl)-1,2-dihydropyridin-2-one
4-({[8-(1-methyl-1H-indol-6-yl)quinoxalin-6-yl]amino}methyl)benzamide
8-(1-methyl-1H-indol-6-yl)-N-{[4-(1H-1,2,3,4-tetrazol-5-yl)phenyl]methyl}quinoxalin-6-amine
N-methyl-8-(1-methyl-1H-indol-6-yl)-N-(pyridin-3-ylmethyl)quinoxalin-6-amine
8-(1-methyl-1H-indol-6-yl)-N-[(8S)-5,6,7,8-tetrahydroisoquinolin-8-yl]quinoxalin-6-amine
8-(1-methyl-1H-indol-6-yl)-N-[(8R)-5,6,7,8-tetrahydroisoquinolin-8-yl]quinoxalin-6-amine
8-(1-methyl-1H-indol-4-yl)-N-[1-(pyridin-3-yl)ethyl]quinoxalin-6-amine
4-{[8-(1-methyl-1H-indol-6-yl)quinoxalin-6-yl]amino}piperidin-2-one
8-(1-methyl-1H-indol-6-yl)-N-{[5-(1H-pyrazol-4-yl)pyridin-3-yl]methyl}quinoxalin-6-amine
N-[(5-bromopyridin-3-yl)methyl]-8-(1-methyl-1H-indol-6-yl)quinoxalin-6-amine
8-(1-methyl-1H-indol-6-yl)-N-(piperidin-4-yl)quinoxalin-6-amine
8-(3-methyl-1-benzofuran-5-yl)-N-{1-[5-(1-methyl-1H-pyrazol-4-yl)pyridin-3-yl]ethyl}quinoxalin-6-amine
8-(1-methyl-1H-indol-6-yl)-N-{[5-(pyrimidin-5-yl)pyridin-3-yl]methyl}quinoxalin-6-amine
N-[(5-aminopyridin-3-yl)methyl]-8-(1-methyl-1H-indol-6-yl)quinoxalin-6-amine
8-(1-methyl-1H-indol-6-yl)-N-{[5-(1H-pyrazol-5-yl)pyridin-3-yl]methyl}quinoxalin-6-amine
8-(3-methyl-1-benzofuran-5-yl)-N-(oxan-4-yl)quinoxalin-6-amine
1-(4-{[8-(1-methyl-1H-indol-6-yl)quinoxalin-6-yl]amino}piperidin-1-yl)ethan-1-one
N-{7-azaspiro[3.5]nonan-1-yl}-8-(1-methyl-1H-indol-6-yl)quinoxalin-6-amine
8-(1-methyl-1H-indol-6-yl)-N-[piperidin-4-yl(pyridin-3-yl)methyl]quinoxalin-6-amine
8-(1-methyl-1H-indol-6-yl)-N-{[5-(morpholin-4-yl)pyridin-3-yl]methyl}quinoxalin-6-amine
8-(3-methyl-1-benzofuran-5-yl)-N-(morpholin-2-ylmethyl)quinoxalin-6-amine
8-(1-methyl-1H-indol-6-yl)-N-[(4-methylpyridin-3-yl)methyl]quinoxalin-6-amine
N-[(4-fluoropyridin-3-yl)methyl]-8-(1-methyl-1H-indol-6-yl)quinoxalin-6-amine
5-({[8-(1-methyl-1H-indol-6-yl)quinoxalin-6-yl]amino}methyl)pyridin-3-ol
3-(7-{[1-(pyridin-3-yl)ethyl]amino}quinoxalin-5-yl)benzene-1-sulfonamide
8-(1-methyl-1H-indol-6-yl)-N-(5,6,7,8-tetrahydroquinoxalin-5-yl)quinoxalin-6-amine
8-(3-methyl-1-benzofuran-5-yl)-N-[(1S)-1-[3-(1-methyl-1H-pyrazol-4-yl)phenyl]ethyl]quinoxalin-6-amine
N-[1-(pyridin-3-yl)ethyl]-8-(quinolin-6-yl)quinoxalin-6-amine
8-(1-methyl-1H-indol-6-yl)-N-[oxan-4-yl(pyridin-3-yl)methyl]quinoxalin-6-amine
8-(1-methyl-1H-indol-6-yl)-N-[(1-methylpiperidin-2-yl)methyl]quinoxalin-6-amine
5-({[8-(1-methyl-1H-indol-6-yl)quinoxalin-6-yl]amino}methyl)-1,2-dihydropyridin-2-one
N-[1-(pyridin-3-yl)ethyl]-8-(quinolin-7-yl)quinoxalin-6-amine
8-(1-methyl-1H-indol-6-yl)-N-{2H,3H,4H-pyrano[3,2-b]pyridin-4-yl}quinoxalin-6-amine
1-[2-({[8-(1-methyl-1H-indol-5-yl)quinoxalin-6-yl]amino}methyl)piperidin-1-yl]ethan-1-one
N-[(2-aminopyrimidin-5-yl)methyl]-8-(1-methyl-1H-indol-6-yl)quinoxalin-6-amine
8-(3-methyl-1-benzofuran-5-yl)-N-{[5-(1-methyl-1H-pyrazol-4-yl)pyridin-3-yl]methyl}quinoxalin-6-amine
1-[4-({[8-(1-methyl-1H-indol-6-yl)quinoxalin-6-yl]amino}(pyridin-3-yl)methyl)piperidin-1-yl]ethan-1-one
N-[(2-chloropyrimidin-5-yl)methyl]-8-(1-methyl-1H-indol-6-yl)quinoxalin-6-amine
8-(1-methyl-1H-indol-6-yl)-N-[(4-methylmorpholin-2-yl)methyl]quinoxalin-6-amine
8-(1-methyl-1H-indol-6-yl)-N-{[4-(pyrimidin-5-yl)pyridin-3-yl]methyl}quinoxalin-6-amine
8-(1-methyl-1H-indol-6-yl)-N-{[4-(4-methylpiperazin-1-yl)pyridin-3-yl]methyl}quinoxalin-6-amine
N-{imidazo[1,2-a]pyridin-6-ylmethyl}-8-(1-methyl-1H-indol-6-yl)quinoxalin-6-amine
8-(1-methyl-1H-indol-6-yl)-N-{[4-(1-methyl-1H-pyrazol-4-yl)pyridin-3-yl]methyl}quinoxalin-6-amine
1-[2-({[8-(1-methyl-1H-indol-6-yl)quinoxalin-6-yl]amino}methyl)morpholin-4-yl]ethan-1-one
8-(1-methyl-1H-indol-6-yl)-N-(morpholin-3-ylmethyl)quinoxalin-6-amine
1-methyl-4-{[8-(1-methyl-1H-indol-6-yl)quinoxalin-6-yl]amino}piperidin-2-one
1-methyl-5-{[8-(1-methyl-1H-indol-6-yl)quinoxalin-6-yl]amino}piperidin-2-one
N-[(1-methyl-1H-imidazol-5-yl)methyl]-8-(1-methyl-1H-indol-6-yl)quinoxalin-6-amine
N-[(4-bromopyridin-2-yl)methyl]-8-(1-methyl-1H-indol-6-yl)quinoxalin-6-amine
8-(1-methyl-1H-indol-6-yl)-N-{[4-(1-methyl-1H-pyrazol-4-yl)pyridin-2-yl]methyl}quinoxalin-6-amine
N-[(2-bromopyridin-4-yl)methyl]-8-(1-methyl-1H-indol-6-yl)quinoxalin-6-amine
8-(1-methyl-1H-indol-6-yl)-N-{[2-(1-methyl-1H-pyrazol-4-yl)pyridin-4-yl]methyl}quinoxalin-6-amine
N-[(1-methyl-1H-1,2,3-triazol-5-yl)(pyridin-3-yl)methyl]-8-(1-methyl-1H-indol-6-yl)quinoxalin-6-amine
8-(1-methyl-1H-indol-6-yl)-N-[(1-methylpiperidin-4-yl)(pyridin-3-yl)methyl]quinoxalin-6-amine
N-[(4-benzylmorpholin-3-yl)methyl]-8-(1-methyl-1H-indol-6-yl)quinoxalin-6-amine
8-(1-methyl-1H-indol-6-yl)-N-{[4-(pyrimidin-5-yl)morpholin-2-yl]methyl}quinoxalin-6-amine
8-(1-methyl-1H-indol-6-yl)-N-[piperidin-4-yl(pyridin-4-yl)methyl]quinoxalin-6-amine
8-(1-methyl-1H-indol-6-yl)-N-[piperidin-4-yl(pyridazin-3-yl)methyl]quinoxalin-6-amine
N-[(4-aminopyridin-3-yl)methyl]-8-(1-methyl-1H-indol-6-yl)quinoxalin-6-amine
N-[(4-methoxypyridin-3-yl)methyl]-8-(l-methyl-1H-indol-6-yl)quinoxalin-6-amine
1-{4-[3-({[8-(1-methyl-1H-indol-6-yl)quinoxalin-6-yl]amino}methyl)pyridin-4-yl]piperazin-1-yl}ethan-1-one
1-[4-({[8-(3-methyl-1-benzofuran-5-yl)quinoxalin-6-yl]amino}(pyridin-3-yl)-methyl)piperidin-1-yl]ethan-1-one
N-[(1-methyl-1H-imidazol-4-yl)(piperid in-4-yl)methyl]-8-(1-methyl-1H-indol-6-yl)quinoxalin-6-amine
N-[(6-methoxypyridin-3-yl)(pyridin-3-yl)methyl]-8-(1-methyl-1H-indol-6-yl)-quinoxalin-6-amine
2-methyl-1-[4-({[8-(1-methyl-1H-indol-6-yl)quinoxalin-6-yl]amino}(pyridin-3-yl)methyl)piperidin-1-yl]propan-1-one
1-[4-({[8-(1-methyl-1H-indol-6-yl)quinoxalin-6-yl]amino}(pyridin-3-yl)-methyl)piperidin-1-yl]propan-1-one
2-[4-({[8-(1-methyl-1H-indol-6-yl)quinoxalin-6-yl]amino}(pyridin-3-yl)-methyl)piperidin-1-yl]acetonitrile
N-[(2-methoxypyridin-4-yl)(pyridin-3-yl)methyl]-8-(1-methyl-1H-indol-6-yl)-quinoxalin-6-amine
8-(1-methyl-1H-indol-6-yl)-N-{1-[4-(1-methyl-1H-pyrazol-4-yl)pyridin-3-yl]-ethyl}quinoxalin-6-amine
8-(1-methyl-1H-indol-6-yl)-N-{1-[4-(4-methylpiperazin-1-yl)pyridin-3-yl]-ethyl}quinoxalin-6-amine
N-[(1-methyl-1H-1,2,3-triazol-5-yl)methyl]-8-(1-methyl-1H-indol-6-yl)quinoxalin-6-amine
5-({[8-(1-methyl-1H-indol-6-yl)quinoxalin-6-yl]amino}(pyridin-3-yl)methyl)-1,2-dihydropyridin-2-one
N-[(1-cyclopropanecarbonylpiperidin-4-yl)(pyridin-3-yl)methyl]-8-(1-methyl-1H-indol-6-yl)quinoxalin-6-amine
8-(1-methyl-1H-indol-6-yl)-N-[pyridin-3-yl(pyridin-4-yl)methyl]quinoxalin-6-amine
1-[4-({[8-(1-methyl-1H-indol-6-yl)quinoxalin-6-yl]amino}(pyridin-3-yl)methyl)-piperidin-1-yl]propan-2-one
1-[4-({[8-(1-methyl-1H-indol-6-yl)quinoxalin-6-yl]amino}(pyridin-3-yl)methyl)-piperidin-1-yl]butan-1-one
1-[3-((S){[8-(1-methyl-1H-indol-6-yl)quinoxalin-6-yl]amino}(pyridin-3-yl)-methyl)piperidin-1-yl]ethan-1-one
1-[3-((R){[8-(1-methyl-1H-indol-6-yl)quinoxalin-6-yl]amino}(pyridin-3-yl)-methyl)piperidin-1-yl]ethan-1-one
3-({[8-(1-methyl-1H-indol-6-yl)quinoxalin-6-yl]amino}methyl)pyridine-4-carbonitrile
2-[4-({[8-(1-methyl-1H-indol-6-yl)quinoxalin-6-yl]amino}(pyridin-3-yl)methyl)-piperidin-1-yl]acetic acid
2-[4-({[8-(1-methyl-1H-indol-6-yl)quinoxalin-6-yl]amino}(pyridin-3-yl)methyl)-piperidin-1-yl]acetamide
1-{4-[(6-methoxypyridin-3-yl)({[8-(1-methyl-1H-indol-6-yl)quinoxalin-6-yl]-amino})methyl]piperidin-1-yl}ethan-1-one
2-methoxy-1-[4-({[8-(1-methyl-1H-indol-6-yl)quinoxalin-6-yl]amino}(pyridin-3-yl)methyl)piperidin-1-yl]ethan-1-one
8-(1-methyl-1H-indol-6-yl)-N-[pyridin-3-yl(pyrimidin-5-yl)methyl]quinoxalin-6-amine
N-[(6-methoxypyridin-3-yl)(pyridin-3-yl)methyl]-8-(3-methyl-1-benzothiophen-5-yl)quinoxalin-6-amine
8-(1-methyl-1H-indol-6-yl)-N-(1,3-oxazol-5-ylmethyl)quinoxalin-6-amine
8-(1-methyl-1H-indol-6-yl)-N-(1,2-thiazol-4-ylmethyl)quinoxalin-6-amine
8-(1-methyl-1H-indol-6-yl)-N-(1,2-oxazol-4-ylmethyl)quinoxalin-6-amine
8-(1-methyl-1H-indol-6-yl)-N-(1,3-thiazol-5-ylmethyl)quinoxalin-6-amine
5-({[8-(3-methyl-1-benzothiophen-5-yl)quinoxalin-6-yl]amino}(pyridin-3-yl)-methyl)-1,2-dihydropyridin-2-one
2-amino-1-[4-({[8-(1-methyl-1H-indol-6-yl)quinoxalin-6-yl]amino}(pyridin-3-yl)-methyl)piperidin-1-yl]ethan-1-one
N-[(1-methyl-1H-imidazol-5-yl)(pyridin-3-yl)methyl]-8-(1-methyl-1H-indol-6-yl)-quinoxalin-6-amine
1-{4-[(1-methyl-1H-1,2,3-triazol-5-yl)({[8-(1-methyl-1H-indol-6-yl)quinoxalin-6-yl]amino})methyl]piperidin-1-yl}ethan-1-one
4-({[8-(1-methyl-1H-indol-6-yl)quinoxalin-6-yl]amino}(pyridin-3-yl)methyl)-1,2-dihydropyridin-2-one
8-(3-methyl-1-benzothiophen-5-yl)-N-[piperidin-4-yl(pyridin-3-yl)methyl]-quinoxalin-6-amine
N-[4-({[8-(1-methyl-1H-indol-6-yl)quinoxalin-6-yl]amino}(pyridin-3-yl)methyl)-cyclohexyl]acetamide
1-[4-({[8-(3-methyl-1-benzothiophen-5-yl)quinoxalin-6-yl]amino}(pyridin-3-yl)-methyl)piperidin-1-yl]ethan-1-one
N—[(S)-(6-methoxypyridin-3-yl)(pyridin-3-yl)methyl]-8-(1-methyl-1H-indol-6-yl)-quinoxalin-6-amine
N—[(R)-(6-methoxypyridin-3-yl)(pyridin-3-yl)methyl]-8-(1-methyl-1H-indol-6-yl)-quinoxalin-6-amine
N,N-dimethyl-3-{[8-(1-methyl-1H-indol-6-yl)quinoxalin-6-yl]amino}-3-(pyridin-3-yl)propanamide
2-amino-1-[4-({[8-(1-methyl-1H-indol-6-yl)quinoxalin-6-yl]amino}(pyridin-3-yl)-methyl)piperidin-1-yl]propan-1-one
N-methyl-2-[4-({[8-(1-methyl-1H-indol-6-yl)quinoxalin-6-yl]amino}(pyridin-3-yl)methyl)piperidin-1-yl]acetamide
N,N-dimethyl-2-[4-({[8-(1-methyl-1H-indol-6-yl)quinoxalin-6-yl]amino}(pyridin-3-yl)methyl)piperidin-1-yl]acetamide
N,N-diethyl-2-[4-({[8-(1-methyl-1H-indol-6-yl)quinoxalin-6-yl]amino}(pyridin-3-yl)methyl)piperidin-1-yl]acetamide
3-amino-1-[4-({[8-(1-methyl-1H-indol-6-yl)quinoxalin-6-yl]amino}(pyridin-3-yl)-methyl)piperidin-1-yl]propan-1-one
8-(1-methyl-1H-indol-6-yl)-N-[(4-methyl-4H-1,2,4-triazol-3-yl)methyl]quinoxalin-6-amine
N-[(3-methyl-1,2thiazol-5-yl)methyl]-8-(1-methyl-1H-indol-6-yl)quinoxalin-6-amine
8-(1-methyl-1H-indol-6-yl)-N-[(1,2-thiazol-5-yl)methyl]quinoxalin-6-amine
N-[(5-methyl-1,3,4-oxadiazol-2-yl)methyl]-8-(1-methyl-1H-indol-6-yl)-quinoxalin-6-amine
N-[(5-methyl-1H-1,2,4-triazol-3-yl)methyl]-8-(1-methyl-1H-indol-6-yl)quinoxalin-6-amine
N-(1H-imidazol-4-ylmethyl)-8-(1-methyl-1H-indol-6-yl)quinoxalin-6-amine
N-[(1,2-dimethyl-1H-imidazol-5-yl)methyl]-8-(1-methyl-1H-indol-6-yl)-quinoxalin-6-amine
8-(1-methyl-1H-indol-6-yl)-N-(4H-1,2,4-triazol-3-ylmethyl)quinoxalin-6-amine
1-[4-({[8-(1-methyl-1H-indol-6-yl)quinoxalin-6-yl]amino}(4-methylpyridin-3-yl)methyl)piperidin-1-yl]ethan-1-one
N-[(2-aminopyridin-4-yl)(pyridin-3-yl)methyl]-8-(1-methyl-1H-indol-6-yl)quinoxalin-6-amine
1-[3-({[8-(1-methyl-1H-indol-6-yl)quinoxalin-6-yl]amino}(pyridin-3-yl)methyl)-azetidin-1-yl]ethan-1-one
N-[(1-methyl-1H-imidazol-4-yl)(pyridin-3-yl)methyl]-8-(1-methyl-1H-indol-6-yl)-quinoxalin-6-amine
1-[4-({[8-(2-amino-1,3-benzothiazol-5-yl)quinoxalin-6-yl]amino}(6-methoxy-pyridin-3-yl)methyl)piperidin-1-yl]ethan-1-one
1-[4-({[8-(4-bromophenyl)quinoxalin-6-yl]amino}(pyridin-3-yl)methyl)piperidin-1-yl]ethan-1-one
1-[4-({[8-(2-amino-1,3-benzothiazol-5-yl)quinoxalin-6-yl]amino}(pyridin-3-yl)-methyl)piperidin-1-yl]ethan-1-one
5-[(1-acetylpiperidin-4-yl)({[8-(1-methyl-1H-indol-6-yl)quinoxalin-6-yl]amino})-methyl]-1-methyl-1,2-dihydropyridin-2-one
8-(2-amino-1,3-benzothiazol-5-yl)-N-[(6-methoxypyridin-3-yl)(pyridin-3-yl)-methyl]quinoxalin-6-amine
N-[(6-aminopyridin-3-yl)(pyridin-3-yl)methyl]-8-(1-methyl-1H-indol-6-yl)-quinoxalin-6-amine
N-[(6-methoxypyridin-3-yl)(pyridin-3-yl)methyl]-N-methyl-8-(1-methyl-1H-indol-6-yl)quinoxalin-6-amine
N-methyl-4-({[8-(1-methyl-1H-indol-6-yl)quinoxalin-6-yl]amino}(pyridin-3-yl)methyl)piperidine-1-carboxamide
N-[(6-methoxypyridin-3-yl)(1-methyl-1H-1,2,3-triazol-5-yl)methyl]-8-(1-methyl-1H-indol-6-yl)quinoxalin-6-amine
N,N-dimethyl-4-({[8-(1-methyl-1H-indol-6-yl)quinoxalin-6-yl]amino}(pyridin-3-yl)methyl)piperidine-1-carboxamide
3-({[8-(1-methyl-1H-indol-5-yl)quinoxalin-6-yl]amino}methyl)benzonitrile
3-({[8-(1-methyl-1H-indol-5-yl)quinoxalin-6-yl]amino}methyl)benzamide
1-(4-{[8-(1-ethyl-1H-indol-6-yl)quinoxalin-6-yl]amino}piperidin-1-yl)ethan-1-one
1-(4-{[8-(1-methyl-1H-indol-5-yl)quinoxalin-6-yl]amino}piperidin-1-yl)ethan-1-one
1-(4-{[8-(1-ethyl-1H-indol-5-yl)quinoxalin-6-yl]amino}piperidin-1-yl)ethan-1-one
1-[4-({8-[3-(dimethylamino)phenyl]quinoxalin-6-yl}amino)piperidin-1-yl]ethan-1-one
N-[(2-chloropyrimidin-5-yl)methyl]-8-(1-methyl-1H-indol-5-yl)quinoxalin-6-amine
1-(4-{[8-(1-benzyl-1H-indol-5-yl)quinoxalin-6-yl]amino}piperidin-1-yl)ethan-1-one
1-(4-{[8-(1-benzyl-1H-indol-6-yl)quinoxalin-6-yl]amino}piperidin-1-yl)ethan-1-one
1-[4-({8-[1-(propan-2-yl)-1H-indol-6-yl]quinoxalin-6-yl}amino)piperidin-1-yl]-ethan-1-one
1-(4-{[8-(1-methyl-1H-indazol-6-yl)quinoxalin-6-yl]amino}piperidin-1-yl)ethan-1-one
1-(3-{[8-(1-methyl-1H-indol-6-yl)quinoxalin-6-yl]amino}pyrrolidin-1-yl)ethan-1-one
1-(3-{[8-(1-methyl-1H-indol-6-yl)quinoxalin-6-yl]amino}azetidin-1-yl)ethan-1-one
1-(4-{[8-(1-methyl-1H-1,3-benzodiazol-6-yl)quinoxalin-6-yl]amino}piperidin-1-yl)ethan-1-one
1-(4-{[8-(2-methyl-2H-indazol-5-yl)quinoxalin-6-yl]amino}piperidin-1-yl)ethan-1-one
N-[(2-aminopyrimidin-5-yl)methyl]-8-(1-methyl-1H-indol-5-yl)quinoxalin-6-amine
1-[(3R)-3-{[8-(1-methyl-1H-indol-6-yl)quinoxalin-6-yl]amino}piperidin-1-yl]-ethan-1-one
1-(5-{7-[(1-acetylpiperidin-4-yl)amino]quinoxalin-5-yl}pyridin-2-yl)ethan-1-one
N-[(5-bromopyridin-3-yl)methyl]-8-(1-methyl-1H-indazol-6-yl)quinoxalin-6-amine
1-[(3S)-3-{[8-(1-methyl-1H-indol-6-yl)quinoxalin-6-yl]amino}pyrrolidin-1-yl]-ethan-1-one
1-[(3S)-3-{[8-(1-methyl-1H-indol-6-yl)quinoxalin-6-yl]amino}piperidin-1-yl]-ethan-1-one
1-[(3S)-3-{[8-(1-methyl-1H-indazol-6-yl)quinoxalin-6-yl]amino}piperidin-1-yl]-ethan-1-one
1-(4-{[8-(1H-1,3-benzodiazol-6-yl)quinoxalin-6-yl]amino}piperidin-1-yl)ethan-1-one
1-[(3R)-3-{[8-(1-methyl-1H-indol-6-yl)quinoxalin-6-yl]amino}pyrrolidin-1-yl]-ethan-1-one
8-(1-methyl-1H-indol-6-yl)-N-(pyrrolidin-3-yl)quinoxalin-6-amine
1-[(3S)-3-{[8-(1-methyl-1H-indazol-6-yl)quinoxalin-6-yl]amino}pyrrolidin-1-yl]-ethan-1-one
1-(4-{[8-(1H-indol-6-yl)quinoxalin-6-yl]amino}piperidin-1-yl)ethan-1-one
1-(4-{[8-(1-methyl-1H-indol-2-yl)quinoxalin-6-yl]amino}piperidin-1-yl)ethan-1-one
3-{7-[(1-acetylpyrrolidin-3-yl)amino]quinoxalin-5-yl}benzamide
1-(4-{[8-(2-methoxypyridin-4-yl)quinoxalin-6-yl]amino}piperidin-1-yl)ethan-1-one
1-(3-{[8-(1-methyl-1H-indol-6-yl)quinoxalin-6-yl]amino}pyrrolidin-1-yl)propan-1-one
1-(3-{[8-(1-methyl-1H-indazol-6-yl)quinoxalin-6-yl]amino}azetidin-1-yl)ethan-1-one
1-[(3S)-3-{[8-(1-methyl-2,3-dihydro-1H-indol-6-yl)quinoxalin-6-yl]amino}-pyrrolidin-1-yl]ethan-1-one
1-(3-{[8-(3-methyl-1-benzothiophen-5-yl)quinoxalin-6-yl]amino}pyrrolidin-1-yl)ethan-1-one
1-(4-{[8-(1-methyl-2,3-dihydro-1H-indol-6-yl)quinoxalin-6-yl]amino}piperidin-1-yl)ethan-1-one
N-(1-benzoylpyrrolidin-3-yl)-8-(1-methyl-1H-indol-6-yl)quinoxalin-6-amine
N-(1-methanesulfonylpyrrolidin-3-yl)-8-(1-methyl-1H-indol-6-yl)quinoxalin-6-amine
2-methyl-1-(3-{[8-(1-methyl-1H-indol-6-yl)quinoxalin-6-yl]amino}pyrrolidin-1-yl)propan-1-one
6-[(1-acetylpyrrolidin-3-yl)amino]-8-(1-methyl-1H-indol-6-yl)quinoxaline-2-carbonitrile
N-(1-cyclopropanecarbonylpyrrolidin-3-yl)-8-(1-methyl-1H-indol-6-yl)quinoxalin-6-amine
1-(3-{[8-(naphthalen-2-yl)quinoxalin-6-yl]amino}pyrrolidin-1-yl)ethan-1-one
1-(3-{[8-(1-methyl-1,2,3,4-tetrahydroquinolin-7-yl)quinoxalin-6-yl]amino}-pyrrolidin-1-yl)ethan-1-one
1-[(3S)-3-({8-[3-(dimethylamino)-4-methylphenyl]quinoxalin-6-yl}amino)-pyrrolidin-1-yl]ethan-1-one
1-(4-{[8-(1-methyl-1H-indol-6-yl)quinoxalin-6-yl]amino}azepan-1-yl)ethan-1-one
N-(1-cyclopropanecarbonylazepan-4-yl)-8-(1-methyl-1H-indol-6-yl)quinoxalin-6-amine
1-[(3S)-3-({8-[4-methyl-3-(methylamino)phenyl]quinoxalin-6-yl}amino)pyrrolidin-1-yl]ethan-1-one
1-[(3S)-3-{[8-(1H-1,3-benzodiazol-2-yl)quinoxalin-6-yl]amino}pyrrolidin-1-yl]ethan-1-one
1-(4-{[8-(1-methyl-1,2,3,4-tetrahydroquinolin-7-yl)quinoxalin-6-yl]amino}-piperidin-1-yl)ethan-1-one
8-(1-methyl-1H-indol-6-yl)-N-[1-(pyridin-3-yl)pyrrolidin-3-yl]quinoxalin-6-amine
8-(1-methyl-1H-indol-6-yl)-N-[(3S)-1-(pyrimidin-4-yl)pyrrolidin-3-yl]quinoxalin-6-amine
8-(1-methyl-1H-indol-6-yl)-N-[(3S)-1-(pyrimidin-2-yl)pyrrolidin-3-yl]quinoxalin-6-amine
1-[(3S)-3-{[8-(5-methyl-2,3,4,5-tetrahydro-1H-1,5-benzodiazepin-7-yl)quinoxalin-6-yl]amino}pyrrolidin-1-yl]ethan-1-one
1-[(3S)-3-{[8-(4-methyl-1,2,3,4-tetrahydroquinoxalin-6-yl)quinoxalin-6-yl]-amino}pyrrolidin-1-yl]ethan-1-one
1-[(3S)-3-({8-[3-(dimethylamino)-4-methoxyphenyl]quinoxalin-6-yl}amino)-pyrrolidin-1-yl]ethan-1-one
8-(1-methyl-1H-indol-6-yl)-N-[(3S)-1-(pyridin-2-yl)pyrrolidin-3-yl]quinoxalin-6-amine
8-(1-methyl-1H-indol-6-yl)-N-[(3S)-1-(pyrimidin-5-yl)pyrrolidin-3-yl]quinoxalin-6-amine
4-[(3S)-3-{[8-(1-methyl-1H-indol-6-yl)quinoxalin-6-yl]amino}pyrrolidin-1-yl]-pyrimidin-2-ol
8-(1-methyl-1H-indol-6-yl)-N-(1-phenylpyrrolidin-3-yl)quinoxalin-6-amine
8-(1-methyl-1H-indol-6-yl)-N-[1-(pyrimidin-2-yl)piperidin-4-yl]quinoxalin-6-amine
8-(1-methyl-1H-indol-6-yl)-N-[(3S)-1-methylpyrrolidin-3-yl]quinoxalin-6-amine
2-amino-1-[(3S)-3-{[8-(1-methyl-1H-indol-6-yl)quinoxalin-6-yl]amino}-pyrrolidin-1-yl]ethan-1-one
8-(1-methyl-1H-indol-6-yl)-N-[(3S)-1-(3-methylpyridin-2-yl)pyrrolidin-3-yl]-quinoxalin-6-amine
1-[(3S)-3-[(8-{3-[ethyl(methyl)amino]-4-methylphenyl}quinoxalin-6-yl)amino]-pyrrolidin-1-yl]ethan-1-one
8-(3-methyl-1H-indol-5-yl)-N-[(3S)-1-(pyrimidin-4-yl)pyrrolidin-3-yl]quinoxalin-6-amine
8-(1,3-dimethyl-1H-indol-5-yl)-N-[(3S)-1-(pyrimidin-2-yl)pyrrolidin-3-yl]quinoxalin-6-amine
8-(1-methyl-1H-indol-6-yl)-N-[(3S)-1-(pyrazin-2-yl)pyrrolidin-3-yl]quinoxalin-6-amine
8-(1-methyl-1H-indol-6-yl)-N-[(3S)-1-(2-methylpyrimidin-4-yl)pyrrolidin-3-yl]-quinoxalin-6-amine
8-(1-methyl-1H-indol-6-yl)-N-[(3S)-1-(pyridine-2-carbonyl)pyrrolidin-3-yl]-quinoxalin-6-amine
N-[(3S)-1-(1H-1,3-benzodiazol-2-yl)pyrrolidin-3-yl]-8-(1-methyl-1H-indol-6-yl)-quinoxalin-6-amine
N-[(1,4-cis)-4-{[8-(1-methyl-1H-indol-6-yl)quinoxalin-6-yl]amino}cyclohexyl]-acetamide
N-(4-methanesulfonylpyridin-2-yl)-8-(1-methyl-1H-indol-6-yl)-N-[(pyridin-3-yl)-methyl]quinoxalin-6-amine
N-(4-methanesulfonylpyridin-3-yl)-8-(1-methyl-1H-indol-6-yl)-N-[(pyridin-3-yl)-methyl]quinoxalin-6-amine
8-(1-methyl-1H-indol-6-yl)-N-(pyridin-3-yl)-N-[(pyridin-4-yl)methyl]quinoxalin-6-amine
N-(1-methyl-1H-1,2,3-triazol-5-yl)-8-(1-methyl-1H-indol-6-yl)-N-[(pyridin-3-yl)-methyl]quinoxalin-6-amine
1-[3-({[8-(1-methyl-1H-indol-6-yl)quinoxalin-6-yl](pyridin-3-yl)amino}-methyl)piperidin-1-yl]ethan-1-one
N-(5-methanesulfonylpyridin-3-yl)-8-(1-methyl-1H-indol-6-yl)-N-[(pyridin-3-yl)-methyl]quinoxalin-6-amine
N-(2-methanesulfonylpyridin-4-yl)-8-(1-methyl-1H-indol-6-yl)-N-[(pyridin-3-yl)-methyl]quinoxalin-6-amine
3-{[8-(1-methyl-1H-indol-6-yl)quinoxalin-6-yl][(pyridin-3-yl)methyl]amino}-pyridine-4-carboxamide
8-(1-methyl-1H-indol-6-yl)-N-[(1-methyl-1H-pyrazol-5-yl)methyl]-quinoxalin-6-amine
N-[(6-methoxypyridin-3-yl)(1-methyl-1H-1,2,3-triazol-5-yl)methyl]-8-(3-methyl-1-benzothiophen-5-yl)quinoxalin-6-amine
8-(1-methyl-1H-indol-6-yl)-N-({8-methyl-8-azabicyclo[3.2.1]octan-3-yl}(pyridin-3-yl)methyl)quinoxalin-6-amine
8-(1-methyl-1H-indol-6-yl)-N-{[6-(methylamino)pyridin-3-yl](pyridin-3-yl)methyl}quinoxalin-6-amine
8-(1-methyl-1H-indol-6-yl)-N-[(1-methyl-1H-pyrazol-4-yl)methyl]quinoxalin-6-amine
N-[5-({[8-(1-methyl-1H-indol-6-yl)quinoxalin-6-yl]amino}(pyridin-3-yl)methyl)-pyridin-2-yl]acetamide
N-[(4-aminocyclohexyl)(pyridin-3-yl)methyl]-8-(1-methyl-1H-indol-6-yl)-quinoxalin-6-amine
N-[bis(6-methoxypyridin-3-yl)methyl]-8-(1-methyl-1H-indol-6-yl)quinoxalin-6-amine
1-{4-[(R)-{[8-(3-methyl-1-benzothiophen-5-yl)quinoxalin-6-yl]amino}(pyridin-3-yl)methyl]piperidin-1-yl}ethan-1-one
1-{4-[(S)-{[8-(3-methyl-1-benzothiophen-5-yl)quinoxalin-6-yl]amino}(pyridin-3-yl)methyl]piperid in-1-yl}ethan-1-one
N-[(2-methyl-1,3-oxazol-5-yl)methyl]-8-(1-methyl-1H-indol-6-yl)quinoxalin-6-amine
8-(3-methyl-1-benzothiophen-5-yl)-N-[(1-methyl-1H-imidazol-5-yl)(pyridin-3-yl)methyl]quinoxalin-6-amine
N-[(6-methoxypyridin-3-yl)(1-methyl-1H-imidazol-5-yl)methyl]-8-(1-methyl-1H-indol-6-yl)quinoxalin-6-amine
N-[(6-methoxypyridin-3-yl)(1-methyl-1H-imidazol-5-yl)methyl]-8-(3-methyl-1-benzothiophen-5-yl)quinoxalin-6-amine
N-[(6-methoxypyridin-3-yl)(1-methyl-1H-1,2,3-triazol-5-yl)methyl]-8-(3-methyl-1-benzofuran-5-yl)quinoxalin-6-amine
N-[(6-methoxypyridin-3-yl)(1-methyl-1H-pyrazol-5-yl)methyl]-8-(1-methyl-1H-indol-6-yl)quinoxalin-6-amine
N-[(1-methanesulfonylpiperidin-4-yl)(pyridin-3-yl)methyl]-8-(1-methyl-1H-indol-6-yl)quinoxalin-6-amine
N-[(6-methoxypyridin-3-yl)(1,2-thiazol-5-yl)methyl]-8-(1-methyl-1H-indol-6-yl)quinoxalin-6-amine
8-(1-methyl-1H-indol-6-yl)-N-{[2-(methylamino)pyridin-4-yl](pyridin-3-yl)methy I}quinoxalin-6-amine
1-methyl-5-({[8-(1-methyl-1H-indol-6-yl)quinoxalin-6-yl]amino}(pyridin-3-yl)-methyl)-1,2-dihydropyridin-2-one
1-[4-(2-{[8-(1-methyl-1H-indol-6-yl)quinoxalin-6-yl]amino}-2-(pyridin-3-yl)-ethyl)piperidin-1-yl]ethan-1-one
N-[(6-methoxypyridin-3-yl)(1,3-oxazol-5-yl)methyl]-8-(1-methyl-1H-indol-6-yl)-quinoxalin-6-amine
8-(1-methyl-1H-indol-6-yl)-N-[2-(1-methylpyrrolidin-3-yl)-1-(pyridin-3-yl)-ethyl]quinoxalin-6-amine
4-({[8-(1-methyl-1H-indol-6-yl)quinoxalin-6-yl]amino}(pyridin-3-yl)methyl)-cyclohexan-1-ol
N-[1,1-bis(pyridin-3-yl)ethyl]-8-(1-methyl-1H-indol-6-yl)quinoxalin-6-amine
N-[4-({[8-(3-methyl-1-benzothiophen-5-yl)quinoxalin-6-yl]amino}(pyridin-3-yl)-methyl)pyridin-2-yl]acetamide
N-[(6-methoxypyridin-3-yl)methyl]-8-(1-methyl-1H-indol-6-yl)quinoxalin-6-amine
N-[(6-methoxypyridin-3-yl)(1-methyl-1H-1,2,3,4-tetrazol-5-yl)methyl]-8-(1-methyl-1H-indol-6-yl)quinoxalin-6-amine
N-[(6-methoxypyridin-3-yl)methyl]-8-(1-methyl-1H-indol-6-yl)quinoxalin-6-amine
8-(1-methyl-1H-indol-6-yl)-N-(pyridazin-4-ylmethyl)quinoxalin-6-amine
N—[(R)-(6-methoxypyridin-3-yl)(1-methyl-1H-1,2,3-triazol-5-yl)methyl]-8-(3-methyl-1-benzothiophen-5-yl)quinoxalin-6-amine
N—[(S)-(6-methoxypyridin-3-yl)(1-methyl-1H-1,2,3-triazol-5-yl)methyl]-8-(3-methyl-1-benzothiophen-5-yl)quinoxalin-6-amine
N—[(R)-(6-methoxypyridin-3-yl)(1-methyl-1H-1,2,3-triazol-5-yl)methyl]-8-(1-methyl-1H-indol-6-yl)quinoxalin-6-amine
N—[(S)-(6-methoxypyridin-3-yl)(1-methyl-1H-1,2,3-triazol-5-yl)methyl]-8-(1-methyl-1H-indol-6-yl)quinoxalin-6-amine
N-[(1R,4r)-4-[(R)-{[8-(1-methyl-1H-indol-6-yl)quinoxalin-6-yl]amino}(pyridin-3-yl)methyl]cyclohexyl]acetamide
N-[(1S,4r)-4-[(S)-{[8-(1-methyl-1H-indol-6-yl)quinoxalin-6-yl]amino}(pyridin-3-yl)methyl]cyclohexyl]acetamide
[8-(1-Methyl-1H-indol-6-yl)-quinoxalin-6-yl]-(1-oxy-pyridin-3-ylmethyl)-amine

As used herein, the following definitions shall apply unless otherwise indicated or defined specifically elsewhere in the description and/or the claims for specific substituents, radicals, groups or moieties.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, such as one or more C═C double bond(s) and/or C≡C triple bond(s), but which is not aromatic (also referred to herein as “carbocycle”, “cycloaliphatic” or “cycloalkyl”), that has a single point of attachment to the rest of the molecule. Unless otherwise specified, aliphatic groups contain 1-8 or 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” (or “carbocycle” or “cycloalkyl”) refers to a monocyclic C₃-C₇ hydrocarbon that is completely saturated or that contains one or more units of unsaturation, but which is not aromatic, that has a single point of attachment to the rest of the molecule. The term “alkyl” usually refers to a saturated and acyclic aliphatic moiety, while the term “alkenyl” usually refers to an unsaturated and acyclic aliphatic moiety with one or more C═C double bonds and the term “alkynyl” usually refers to an aliphatic and acyclic moiety with one or more C≡C triple bonds. Exemplary aliphatic groups are linear or branched, substituted or unsubstituted C₁₋₈-alkyl, C₁₋₆-alkyl, C₁₋₄-alkyl, C₂₋₈-alkenyl, C₂₋₆-alkenyl, C₂₋₈-alkynyl, C₂₋₆-alkynyl groups and hybrids thereof such as (cycloalkyl)alkyl, (cycloalkenyl)alkyl or (cycloalkyl)alkenyl.

In particular, the term “C₁₋₃-alkyl” refers to alkyl groups, i.e. saturated acyclic aliphatic groups, having 1, 2 or 3 carbon atoms. Exemplary C₁₋₃-alkyl groups are methyl, ethyl, propyl and isopropyl. The term “C₁₋₄-alkyl” refers to alkyl groups having 1, 2, 3 or 4 carbon atoms. Exemplary C₁₋₄-alkyl groups are methyl, ethyl, propyl, isopropyl, butyl, isobutyl, and tert-butyl. The term “C₁₋₆-alkyl” refers to alkyl groups having 1, 2, 3, 4, 5 or 6 carbon atoms. Exemplary C₁₋₆-alkyl groups are methyl, ethyl, propyl, isopropyl, butyl, isobutyl, tert-butyl, n-pentyl, 2-pentyl, n-hexyl, and 2-hexyl. The term “C₁₋₈-alkyl” refers to alkyl groups having 1, 2, 3, 4, 5, 6, 7, or 8 carbon atoms. Exemplary C₁₋₈-alkyl groups are methyl, ethyl, propyl, isopropyl, butyl, isobutyl, tert-butyl, n-pentyl, 2-pentyl, n-hexyl, 2-hexyl n-heptyl, 2-heptyl, n-octyl, 2-octyl, and 2,2,4-trimethylpentyl. Each of these alkyl groups may be straight-chain or—except for C₁-alkyl and C₂-alkyl—branched; they may be unsubstituted. However, in certain instances, which instances are usually specifically indicated in the definition of specific radicals, residues, groups or substituents elsewhere in this specification and/or the accompanying claims, each of these alkyl groups may be substituted with 1, 2 or 3 substituents that may be the same or different; typical examples of these substituents include but are not limited to halogen, hydroxy, alkoxy, unsubstituted or mono- or disubstituted amino.

In some instances, which instances are usually specifically indicated in the definition of specific radicals, residues, groups or substituents elsewhere in this specification and/or the accompanying claims, the C₁₋₃-alkyl, C₁₋₄-alkyl, C₁₋₆-alkyl, C₁₋₈-alkyl groups may also comprise those residues in which 1 or 2 of non-terminal and non-adjacent —CH₂— (methylene) groups are replaced by —O—, —S— and/or 1 or 2 non-terminal and non-adjacent —CH₂— or —CH— groups are replaced by —NH— or —N—. These replacements yield, for instance, alkyl groups like —CH₂—CH₂—O—CH₃, —CH₂—CH₂—CH₂—S—CH₃, CH₂—CH₂—NH—CH₂—CH₃, CH₂—CH₂—O—CH₂—CH₂—O—CH₃, CH₂—CH₂—N(CH₃)—CH₂—CH₃, and the like. Further and/or different replacements of —CH— and —CH₂— groups may be defined for specific alkyl substituents or radicals elsewhere in the description and/or the claims.

The term “C₃₋₇-cycloalkyl” refers to a cycloaliphatic hydrocarbon, as defined above, with 3, 4, 5, 6 or 7 ring carbon atoms. C₃₋₇-cycloalkyl groups may be unsubstituted or substituted with—unless specified differently elsewhere in this specification—1, 2 or 3 substituents that may be the same of different and are—unless specified differently elsewhere in this specification—selected from the group comprising C₁₋₆-alkyl, O—C₁₋₆-alkyl (alkoxy), halogen, hydroxy unsubstituted or mono- or disubstituted amino. Exemplary C₃₋₇-cycloalkyl groups are cyclopropyl, 2-methyl-cyclopropyl, cyclopropenyl, cyclobutyl, cyclobutenyl, cyclopentyl, cyclopentenyl, cyclohexyl, cyclohexenyl, cycloheptyl, cycloheptenyl.

The term “alkoxy” refers to alkyl substituents and residues that are connected to another structural moiety via an oxygen atom (—O—). Sometimes, it is also referred to as “O-alkyl” and more specifically as “O—C₁₋₄-alkyl”, “O—C₁₋₆-alkyl”, “O—C₁₋₈-alkyl”. Like the similar alkyl groups, it may be straight-chain or—except for —O—C₁-alkyl and —O—C₂-alkyl—branched and may be unsubstituted or substituted with 1, 2 or 3 substituents that may be the same or different and are, if not specified differently elsewhere in this specification, selected from the group comprising halogen, unsubstituted or mono- or disubstituted amino. Exemplary alkoxy groups are methoxy, trifluoromethoxy, ethoxy, 2,2,2-trifluoroethoxy, n-propoxy, iso-propoxy, n-butoxy, sec-butoxy, tert-butoxy, n-pentoxy.

The term “alkylene” refers to a divalent alkyl group. An “alkylene chain” is a polymethylene group, i.e., —(CH₂)ₙ—, wherein n is a positive integer, preferably 1, 2, 3, 4, 5 or 6. In the context of the present invention “C₁₋₃-alkylene” refers to an alkylene moiety with 1, 2 and 3, respectively, —CH₂— groups; the term “alkylene”, however, not only comprises linear alkylene groups, i.e. “alkylene chains”, but branched alkylene groups as well. The term “C₁₋₆-alkylene” refers to an alkylene moiety that is either linear, i.e. an alkylene chain, or branched and has 1, 2, 3, 4, 5 or 6 carbon atoms. A substituted alkylene chain is a polymethylene group in which one or more methylene hydrogen atoms are replaced by (or with) a substituent. Suitable substituents include those described herein for a substituted alkyl group. In some instances 1 or 2 non-adjacent methylene groups of the alkylene chain may be replaced by, for instance, O, S and/or NH or N—C₁₋₄-alkyl. Exemplary alkylene groups are —CH₂—, —CH₂—CH₂—, —CH₂—CH₂—CH₂—CH₂—, —O—CH₂—O—, —O—CH₂—CH₂—O—, —CH₂—NH—CH₂—CH₂—, —CH₂—N(CH₃)—CH₂—CH₂—.

The term “halogen” means F, Cl, Br, or I.

The term “heteroatom” means one or more of oxygen (O), sulfur (S), or nitrogen (N), including, any oxidized form of nitrogen or sulfur, e.g. N-oxides, sulfoxides and sulfones; the quaternized form of any basic nitrogen or a substitutable nitrogen of a heterocyclic or heteroaromatic ring, for example N (as in 3,4-dihydro-2H-pyrrolyl), NH (as in pyrrolidinyl) or N-SUB with SUB being a suitable substituent (as in N-substituted pyrrolidinyl).

The term “aryl” used alone or as part of a larger moiety as in “aralkyl”, “aralkoxy”, or “aryloxyalkyl”, refers to monocyclic, bicyclic and tricyclic ring systems having a total of five to fourteen ring members, that ring members being carbon atoms, wherein at least one ring in the system is aromatic, i.e., it has (4n+2) π (pi) electrons (with n being an integer selected from 0, 1, 2, 3), which electrons are delocalized over the system, and wherein each ring in the system contains three to seven ring members. Preferably, all rings in the aryl system or the entire ring system are aromatic. The term “aryl” is used interchangeably with the term “aryl ring”. In certain embodiments of the present invention, “aryl” refers to an “aromatic ring system”. More specifically, those aromatic ring systems may be mono-, bi- or tricyclic with 5, 6, 7, 8, 9, 10, 11, 12, 13, 14 ring carbon atoms. Even more specifically, those aromatic ring systems may be mono- or bicyclic with 6, 7, 8, 9, 10 ring carbon atoms. Exemplary aryl groups are phenyl, biphenyl, naphthyl, anthracyl and the like, which may be unsubstituted or substituted with one or more identical or different substituents. Also included within the scope of the terms “aryl” or “aromatic ring system”, as they are used herein, is a group in which an aromatic ring is fused to one or more non-aromatic rings, such as indanyl, phthalimidyl, naphthimidyl, phenanthridinyl, or tetrahydronaphthyl, and the like. In the latter case the “aryl” group or substituent is attached to its pendant group via the aromatic part of the ring system.

The terms “heteroaryl” and “heteroar-”, used alone or as part of a larger moiety, e.g., “heteroaralkyl”, or “heteroaralkoxy”, refer to groups having 3, 4, 5, 6, 7, 8, 9, 10, 11, 12, 13, 14 ring atoms (which atoms are carbon and hetero atoms), preferably 5, 6, or 9 ring atoms; having 6, 10, or 14 π (pi) electrons shared in a cyclic array; and having, in addition to carbon atoms, 1, 2, 3, 4 or 5 heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, furazanyl, pyridyl, pyridazinyl, pyrimidinyl, pyrazinyl, indolizinyl, purinyl, naphthyridinyl, pteridinyl, and pyrrolopyridinyl, in particular pyrrolo[2,3-b]pyridinyl. The terms “heteroaryl” and “heteroar-”, as used herein, also include groups in which a heteroaromatic ring is fused to one or more aryl, cycloaliphatic, or heterocyclyl rings, where the radical or point of attachment is preferably on the heteroaromatic or, if present, the aryl ring. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. For example, an indolyl ring may be attached via one of the ring atoms of the six-membered aryl ring or via one of the ring atoms of the five-membered heteroaromatic ring. A heteroaryl group is optionally mono-, bi- or tricyclic. The term “heteroaryl” is used interchangeably with the terms “heteroaryl ring”, “heteroaryl group”, or “heteroaromatic”, any of which terms include rings that are unsubstituted or substituted with one or more identical or different substituents. The term “heteroaralkyl” refers to an alkyl group substituted by a heteroaryl, wherein the alkyl and heteroaryl portions independently are optionally substituted.

A heteroaryl ring can be attached to its pendant group at any of its hetero or carbon ring atoms which attachment results in a stable structure or molecule: any of the ring atoms may be unsubstituted or substituted.

The structures of typical examples of “heteroaryl” substituents as used in the present invention are depicted below:

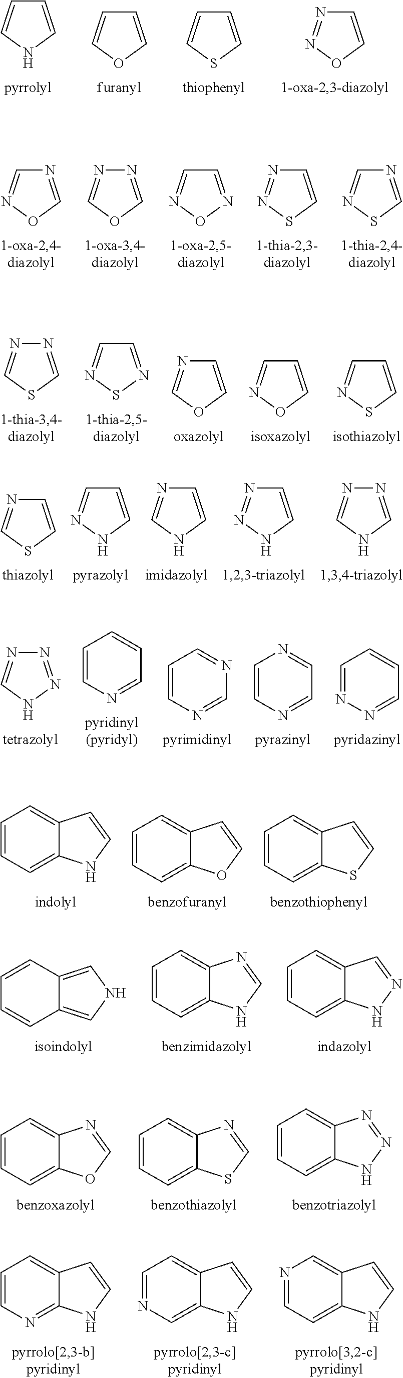

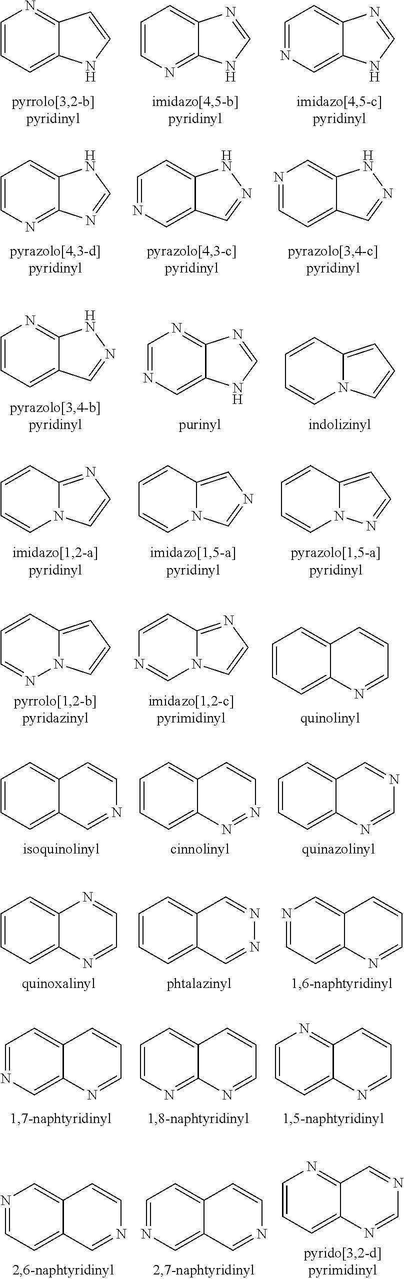

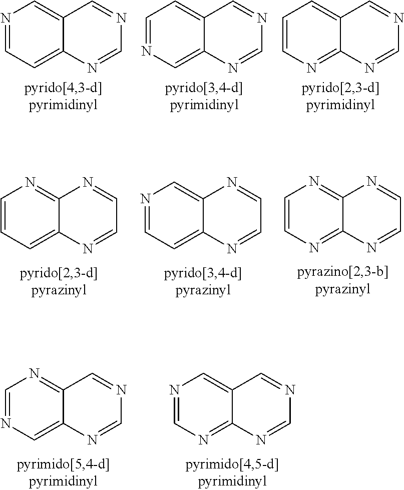

Those heteroaryl substituents can be attached to any pendant group via any of its ring atoms suitable for such an attachment.

As used herein, the terms “heterocycle”, “heterocyclyl”, “heterocyclic radical”, and “heterocyclic ring” are used interchangeably and refer to a stable mono-bi- or tricyclic heterocyclic moiety with 5, 6, 7, 8, 9, 10, 11, 12, 13, 14 ring atoms wherein 1, 2, 3, 4, 5 of said ring atoms are hetero atoms and wherein that heterocyclic moiety is either saturated or partially unsaturated. Preferably, the heterocycle is a stable saturated or partially unsaturated 3-, 4-, 5-, 6-, or 7-membered monocyclic or 7-, 8-, 9-, 10-, or 11-membered bicyclic or 11-, 12-, 13-, or 14-membered tricyclic heterocyclic moiety.

When used in reference to a ring atom of a heterocycle, the term “nitrogen” includes a substituted nitrogen. As an example, in a saturated or partially unsaturated ring having 1-3 heteroatoms selected from oxygen, sulfur or nitrogen, the nitrogen is N (as in 3,4-dihydro-2H-pyrrolyl), NH (as in pyrrolidinyl), or N-SUB with SUB being a suitable substituent (as in N-substituted pyrrolidinyl).

In the context of the term “heterocycle” the term “saturated” refers to a completely saturated heterocyclic system, like pyrrolidinyl, piperidinyl, morpholinyl, and piperidinonyl. With regard to the term “heterocycle” the term “partially unsaturated” refers to heterocyclic systems (i) that contain one or more units of unsaturation, e.g. a C═C or a C=Heteroatom bond, but that are not aromatic, for instance, tetrahydropyridinyl; or (ii) in which a (saturated or unsaturated but non-aromatic) heterocyclic ring is fused with an aromatic or heteroaromatic ring system, wherein, however, the “partially unsaturated heterocycle” is attached to the rest of the molecule (its pendant group) via one of the ring atoms of the “heterocyclic” part of the system and not via the aromatic or heteroaromatic part. This first class (i) of “partially unsaturated” heterocycles may also be referred to as “non-aromatic partially unsaturated” heterocycles. This second class (ii) of “partially unsaturated” heterocycles may also be referred to as (bicyclic or tricyclic) “partially aromatic” heterocycles indicating that at least one of the rings of that heterocycle is a saturated or unsaturated but non-aromatic heterocycle that is fused with at least one aromatic or heteroaromatic ring system. Typical examples of these “partially aromatic” heterocycles are 1,2,3,4-tetrahydroquinolinyl and 1,2,3,4-tetrahydroisoquinolinyl.

A heterocyclic ring can be attached to its pendant group at any heteroatom or carbon atom that results in a stable structure and any of the ring atoms may be unsubstituted or substituted. Examples of such saturated or partially unsaturated heterocyclic radicals include, without limitation, tetrahydrofuranyl, tetrahydropyranyl, tetrahydrothiophenyl pyrrolidinyl, piperidinyl, pyrrolinyl, morpholinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle”, “heterocyclyl”, “heterocyclyl ring”, “heterocyclic group”, “heterocyclic moiety”, and “heterocyclic radical”, are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl, where the radical or point of attachment is on the heterocyclyl ring. A heterocyclyl group is optionally mono-, bi- or tricyclic. The term “heterocyclylalkyl” refers to an alkyl group substituted by a heterocyclyl, wherein the alkyl and heterocyclyl portions independently are unsubstituted or substituted.

The term “unsaturated”, as used herein, means that a moiety has one or more units of unsaturation.

As used herein with reference to any rings, ring systems, ring moieties, and the like, the term “partially unsaturated” refers to a ring moiety that includes at least one double or triple bond. The term “partially unsaturated” is intended to encompass rings having multiple sites of unsaturation. In particular, it encompasses (i) non-saturated (mono-, bi- or tricyclic) ring systems without any aromatic or heteroaromatic moiety or part; and (ii) bi- or tricyclic ring systems in which one of the rings of that system is an aromatic or heteroaromatic ring which is fused with another ring that is neither an aromatic nor a heteroaromatic ring, e.g. tetrahydronaphthyl or tetrahydroquinolinyl. The first class (i) of “partially unsaturated” rings, ring systems, ring moieties may also be referred to as “non-aromatic partially unsaturated” rings, ring systems, ring moieties, while the second class (ii) may be referred to as “partially aromatic” rings, ring systems, ring moieties.

As described herein, certain compounds of the invention contain “substituted” or “optionally substituted” moieties. In general, the term “substituted”, whether preceded by the term “optionally” or not, means that one or more hydrogens of the designated moiety are replaced with a suitable substituent. “Substituted” applies to one or more hydrogens that are either explicit or implicit from the structure. Unless otherwise indicated, a “substituted” or “optionally substituted” group has a suitable substituent at each substitutable position of the group, and when more than one position in any given structure is substituted with more than one substituent selected from a specified group, the substituent is either the same or different at every position. If a certain group, substituent, moiety or radical is “mono-substituted”, it bears one (1) substituent. If it is “di-substituted”, it bears two (2) substituents, being either the same or different; if it is “trisubstituted”, it bears three (3) substituents, wherein all three are the same or two are the same and the third is different or all three are different from each other. Combinations of substituents envisioned by this invention are preferably those that result in the formation of stable or chemically feasible compounds. The term “stable”, as used herein, refers to compounds that are not substantially altered when subjected to conditions to allow for their production, detection, and, in certain embodiments, their recovery, purification, and use for one or more of the purposes disclosed herein.

In the context of the present invention the term “derivative” means any non-toxic salt, ester, salt of an ester or other derivative of a compound of this invention that, upon administration to a recipient, is capable of providing, either directly or indirectly, a compound of this invention or an inhibitorily active metabolite or residue thereof.

The compounds of the present invention can be in the form of a prodrug compound. “Prodrug” and “prodrug compound” mean a derivative that is converted into a biologically active compound according to the present invention under physiological conditions in the living body, e.g., by oxidation, reduction, hydrolysis or the like, each of which is carried out enzymatically, or without enzyme involvement. Examples of prodrugs are compounds, in which the amino group in a compound of the present invention is acylated, alkylated or phosphorylated, e.g., eicosanoylamino, alanylamino, pivaloyloxymethylamino or in which the hydroxyl group is acylated, alkylated, phosphorylated or converted into the borate, e.g. acetyloxy, palmitoyloxy, pivaloyloxy, succinyloxy, fumaryloxy, alanyloxy or in which the carboxyl group is esterified or amidated, or in which a sulfhydryl group forms a disulfide bridge with a carrier molecule, e.g. a peptide, that delivers the drug selectively to a target and/or to the cytosol of a cell. These compounds can be produced from compounds of the present invention according to well-known methods. Other examples of prodrugs are compounds, wherein the carboxylate in a compound of the present invention is for example converted into an alkyl-, aryl-, choline-, amino-, acyloxymethylester, linolenoyl-ester.

The term “solvates” means addition forms of the compounds of the present invention with solvents, preferably pharmaceutically acceptable solvents, that contain either stoichiometric or non stoichiometric amounts of solvent. Some compounds have a tendency to trap a fixed molar ratio of solvent molecules in the crystalline solid state, thus forming a solvate. If the solvent is water the solvate formed is a hydrate, e.g. a mono- or dihydrate. If the solvent is alcohol, the solvate formed is an alcoholate, e.g., a methanolate or ethanolate. If the solvent is an ether, the solvate formed is an etherate, e.g., diethyl etherate.

The term “N-oxides” means such compounds of the present invention that contain an amine oxide moiety, i.e. the oxide of a tertiary amine group.

The compounds of formula (I) may have one or more centres of chirality. They may accordingly occur in various enantiomeric and diastereomeric forms, as the case may be, and be in racemic or optically active form. The invention, therefore, also relates to the optically active forms, enantiomers, racemates, diastereomers, mixtures thereof in all ratios, collectively: “stereoisomers” for the purpose of the present invention, of these compounds. Since the pharmaceutical activity of the racemates or stereoisomers of the compounds according to the invention may differ, it may be desirable to use a specific stereoisomer, e.g. one specific enantiomer or diastereomer. In these cases, a compound according to the present invention obtained as a racemate—or even intermediates thereof—may be separated into the stereoisomeric (enantiomeric, diastereoisomeric) compounds by chemical or physical measures known to the person skilled in the art. Another approach that may be applied to obtain one or more specific stereoisomers of a compound of the present invention in an enriched or pure form makes use of stereoselective synthetic procedures, e.g. applying starting material in a stereoisomerically enriched or pure form (for instance using the pure or enriched (R)- or (S)-enantiomer of a particular starting material bearing a chiral center) or utilizing chiral reagents or catalysts, in particular enzymes. In the context of the present invention the term “pure enantiomer” usually refers to a relative purity of one enantiomer over the other (its antipode) of equal to or greater than 95%, preferably ≥98%, more preferably ≥98.5%, still more preferably ≥99%.

Thus, for example, the compounds of the invention which have one or more centers of chirality and which occur as racemates or as mixtures of enantiomers or diastereoisomers can be fractionated or resolved by methods known per se into their optically pure or enriched isomers, i.e. enantiomers or diastereomers. The separation of the compounds of the invention can take place by chromatographic methods, e.g. column separation on chiral or nonchiral phases, or by recrystallization from an optionally optically active solvent or by use of an optically active acid or base or by derivatization with an optically active reagent such as, for example, an optically active alcohol, and subsequent elimination of the radical.

In the context of the present invention the term “tautomer” refers to compounds of the present invention that may exist in tautomeric forms and show tautomerism; for instance, carbonyl compounds may be present in their keto and/or their enol form and show keto-enol tautomerism. Those tautomers may occur in their individual forms, e.g., the keto or the enol form, or as mixtures thereof and are claimed separately and together as mixtures in any ratio. The same applies for cis/trans isomers, E/Z isomers, conformers and the like.

The compounds of the present invention can be in the form of a pharmaceutically acceptable salt, a pharmaceutically acceptable solvate, or a pharmaceutically acceptable solvate of a pharmaceutically acceptable salt.

The term “pharmaceutically acceptable salts” refers to salts prepared from pharmaceutically acceptable bases or acids, including inorganic bases or acids and organic bases or acids. In cases where the compounds of the present invention contain one or more acidic or basic groups, the invention also comprises their corresponding pharmaceutically acceptable salts. Thus, the compounds of the present invention which contain acidic groups can be present in salt form, and can be used according to the invention, for example, as alkali metal salts, alkaline earth metal salts or as ammonium salts. More precise examples of such salts include sodium salts, potassium salts, calcium salts, magnesium salts or salts with ammonia or organic amines such as, for example, ethylamine, ethanolamine, triethanolamine or amino acids. Compounds of the present invention which contain one or more basic groups, e.g. groups which can be protonated, can be present in salt form, and can be used according to the invention in the form of their addition salts with inorganic or organic acids. Examples of suitable acids include hydrogen chloride, hydrogen bromide, hydrogen iodide, phosphoric acid, sulfuric acid, nitric acid, methanesulfonic acid, p-toluenesulfonic acid, naphthalenedisulfonic acid, sulfoacetic acid, trifluoroacetic acid, oxalic acid, acetic acid, tartaric acid, lactic acid, salicylic acid, benzoic acid, carbonic acid, formic acid, propionic acid, pivalic acid, diethylacetic acid, malonic acid, succinic acid, pimelic acid, fumaric acid, malonic acid, maleic acid, malic acid, embonic acid, mandelic acid, sulfaminic acid, phenylpropionic acid, gluconic acid, ascorbic acid, isonicotinic acid, citric acid, adipic acid, taurocholic acid, glutaric acid, stearic acid, glutamic acid or aspartic acid, and other acids known to the person skilled in the art. The salts which are formed are, inter alia, hydrochlorides, chlorides, hydrobromides, bromides, iodides, sulfates, phosphates, methanesulfonates (mesylates), tosylates, carbonates, bicarbonates, formates, acetates, sulfoacetates, triflates, oxalates, malonates, maleates, succinates, tartrates, malates, embonates, mandelates, fumarates, lactates, citrates, glutarates, stearates, aspartates and glutamates. The stoichiometry of the salts formed from the compounds of the invention may moreover be an integral or non-integral multiple of one.

If the compounds of the present invention simultaneously contain acidic and basic groups in the molecule, the invention also includes, in addition to the salt forms mentioned, inner salts or betaines (zwitterions). The respective salts can be obtained by customary methods which are known to a person skilled in the art, for example by contacting these with an organic or inorganic acid or base in a solvent or dispersant, or by anion exchange or cation exchange with other salts. The present invention also includes all salts of the compounds of the present invention which, owing to low physiological compatibility, are not directly suitable for use in pharmaceuticals but which can be used, for example, as intermediates for chemical reactions or for the preparation of pharmaceutically acceptable salts.

Therefore, the following items are also in accordance with the invention:
(a) all stereoisomers or tautomers of the compounds, including mixtures thereof in all ratios;
(b) prodrugs of the compounds, or stereoisomers or tautomers of these prodrugs;
(c) pharmaceutically acceptable salts of the compounds and of the items mentioned under (a) and (b);
(d) pharmaceutically acceptable solvates of the compounds and of the items mentioned under (a), (b) and (c);
(e) N-oxides of the compounds and of the items mentioned under (a), (b), (c), and (d).

It should be understood that all references to compounds above and below are meant to include these items, in particular pharmaceutically acceptable solvates of the compounds, or pharmaceutically acceptable solvates of their pharmaceutically acceptable salts.

Furthermore, the present invention relates to pharmaceutical compositions comprising at least one compound of formula (I), or its derivatives, prodrugs, solvates, tautomers or stereoisomers thereof as well as the physiologically acceptable salts of each of the foregoing, including mixtures thereof in all ratios, as active ingredient, together with a pharmaceutically acceptable carrier.

For the purpose of the present invention the term “pharmaceutical composition” refers to a composition or product comprising one or more active ingredients, and one or more inert ingredients that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing at least one compound of the present invention and a pharmaceutically acceptable carrier. It may further comprise physiologically acceptable excipients, auxiliaries, adjuvants, diluents and/or additional pharmaceutically active substance other than the compounds of the invention.

The pharmaceutical compositions include compositions suitable for oral, rectal, topical, parenteral (including subcutaneous, intramuscular, and intravenous), ocular (ophthalmic), pulmonary (nasal or buccal inhalation), or nasal administration, although the most suitable route in any given case will depend on the nature and severity of the conditions being treated and on the nature of the active ingredient. They may be conveniently presented in unit dosage form and prepared by any of the methods well-known in the art of pharmacy.

A pharmaceutical composition of the present invention may additionally comprise one or more other compounds as active ingredients (drugs), such as one or more additional compounds of the present invention. In a particular embodiment the pharmaceutical composition further comprises a second active ingredient or its derivatives, prodrugs, solvates, tautomers or stereoisomers thereof as well as the physiologically acceptable salts of each of the foregoing, including mixtures thereof in all ratios, wherein that second active ingredient is other than a compound of formula (I); preferably, that second active ingredient is a compound that is useful in the treatment, prevention, suppression and/or amelioration of medicinal conditions or pathologies for which the compounds of the present invention are useful as well and which are listed elsewhere hereinbefore or hereinafter. Such combination of two or more active ingredients or drugs may be safer or more effective than either drug or active ingredient alone, or the combination is safer or more effective than it would be expected based on the additive properties of the individual drugs. Such other drug(s) may be administered, by a route and in an amount commonly used contemporaneously or sequentially with a compound of the invention. When a compound of the invention is used contemporaneously with one or more other drugs or active ingredients, a combination product containing such other drug(s) and the compound of the invention—also referred to as “fixed dose combination”—is preferred. However, combination therapy also includes therapies in which the compound of the present invention and one or more other drugs are administered on different overlapping schedules. It is contemplated that when used in combination with other active ingredients, the compound of the present invention or the other active ingredient or both may be used effectively in lower doses than when each is used alone. Accordingly, the pharmaceutical compositions of the present invention include those that contain one or more other active ingredients, in addition to a compound of the invention.

The compounds of the present invention can be used as medicaments. They exhibit pharmacological activity by inhibiting 6-phosphofructo-2-kinase/fructose-2,6-bisphosphatase (PFKFB), in particular its isoforms PFKFB3 and/or PFKFB4, more particular PFKFB3. Even more particular, the compounds of the present invention exhibit inhibition of the kinase enzymatic activity of PFKFB, especially of PFKFB3 and/or PFKFB4, more especially of PFKFB3. Thus, they are useful for the treatment, prevention, suppression and/or amelioration of medicinal conditions or pathologies that are affected by PFKFB activity, in particular by PFKFB3 and/or PFKFB4 activity, more particular by PFKFB3 activity. The compounds of the present invention are thus particularly useful for the treatment of a hyperproliferative disorder. More specifically, they are useful for the treatment of a disorder or disease selected from the group consisting of cancer, in particular adipose cancer, anogenital cancer, bladder cancer, breast cancer, central nervous system cancer, cervical cancer, colon cancer, connective tissue cancer, glioblastoma, glioma, kidney cancer, leukemia, lung cancer, lymphoid cancer, ovarian cancer, pancreatic cancer, prostate cancer, retinal cancer, skin cancer, stomach cancer, uterine cancer.

Furthermore, some of the compounds of formula (I) may not only exhibit inhibiting activity on PFKFB but further exhibit activity by modulating the activity of other pharmacological target molecules than PFKFB, for instance autotaxin, Brk, BTK, cyclophilin, ERK, Gcn2, hexokinase I, hexokinase II, IKK-epsilon, IRAK1, IRAK4, Ire1, JNK, LDHA/B, LPA, PDK-1, TGF-beta or VEGF target molecules which modulating activity may be useful for the treatment of one or more of the hyperproliferative disorders mentioned above. Thus, those compounds of formula (I) exhibiting activity on PFKFB and another pharmacological target may also be described as having a dual mode of action and may allow for targeting two different target molecules involved in the genesis and progression of a hyperproliferative disorder, in particular cancer.

Compounds of the present invention that exhibit inhibiting activity on PFKFB and modulating, in particular inhibiting activity on another pharmacological target molecule at the same time may exhibit more pronounced activity on one of the targets, usually on PFKFB, than on the other target on which they are active, or, in a few instances, they may exhibit the same or nearly the same activity on both targets (in term of, e.g., IC50 values). While the vast majority of the compounds of the present invention is more active on PFKFB than on any other target, if any, several compounds of the present invention may be to some extent more active on a target other than PFKFB, like one of those mentioned above, e.g. BRK. BRK (breast cancer kinase; also known as PTK6), for instance, is a tyrosine kinase reported to have a significantly higher total activity in malignant mammary tissue than in normal mammary tissue which makes it an attractive target for the prevention and/or treatment of certain cancer diseases, in particular breast cancer (H. A. Hussain, A. J. Harvey, World J Clin Oncol 2014 Aug. 10; 5(3): 299-310).

The disclosed compounds of the formula (I) can be administered and/or used in combination with other known therapeutic agents, including anticancer agents. As used herein, the term “anticancer agent” relates to any agent which is administered to a patient with cancer for the purposes of treating the cancer.

The anti-cancer treatment defined above may be applied as a monotherapy or may involve, in addition to the herein disclosed compounds of formula (I), conventional surgery or radiotherapy or medicinal therapy. Such medicinal therapy, e.g. a chemotherapy or a targeted therapy, may include one or more, but preferably one, of the following anti-tumor agents:

### Alkylating Agents

such as altretamine, bendamustine, busulfan, carmustine, chlorambucil, chlormethine, cyclophosphamide, dacarbazine, ifosfamide, improsulfan, tosilate, lomustine, melphalan, mitobronitol, mitolactol, nimustine, ranimustine, temozolomide, thiotepa, treosulfan, mechloretamine, carboquone; apaziquone, fotemustine, glufosfamide, palifosfamide, pipobroman, trofosfamide, uramustine, TH-302⁴, VAL-083⁴;

### Platinum Compounds

such as carboplatin, cisplatin, eptaplatin, miriplatine hydrate, oxaliplatin, lobaplatin, nedaplatin, picoplatin, satraplatin;

### DNA Altering Agents

such as amrubicin, bisantrene, decitabine, mitoxantrone, procarbazine, trabectedin, clofarabine;
amsacrine, brostallicin, pixantrone, laromustine^{1,3};

### Topoisomerase Inhibitors

such as etoposide, irinotecan, razoxane, sobuzoxane, teniposide, topotecan; amonafide, belotecan, elliptinium acetate, voreloxin;

### Microtubule Modifiers

such as cabazitaxel, docetaxel, eribulin, ixabepilone, paclitaxel, vinblastine, vincristine, vinorelbine, vindesine, vinflunine;
fosbretabulin, tesetaxel;

### Antimetabolites

such as asparaginase³, azacitidine, calcium levofolinate, capecitabine, cladribine, cytarabine, enocitabine, floxuridine, fludarabine, fluorouracil, gemcitabine, mercaptopurine, methotrexate, nelarabine, pemetrexed, pralatrexate, azathioprine, thioguanine, carmofur;
doxifluridine, elacytarabine, raltitrexed, sapacitabine, tegafur^{2,3}, trimetrexate;

### Anticancer Antibiotics

such as bleomycin, dactinomycin, doxorubicin, epirubicin, idarubicin, levamisole, miltefosine, mitomycin C, romidepsin, streptozocin, valrubicin, zinostatin, zorubicin, daunurobicin, plicamycin;
aclarubicin, peplomycin, pirarubicin;

### Hormones/Antagonists

such as abarelix, abiraterone, bicalutamide, buserelin, calusterone, chlorotrianisene, degarelix, dexamethasone, estradiol, fluocortolone fluoxymesterone, flutamide, fulvestrant, goserelin, histrelin, leuprorelin, megestrol, mitotane, nafarelin, nandrolone, nilutamide, octreotide, prednisolone, raloxifene, tamoxifen, thyrotropin alfa, toremifene, trilostane, triptorelin, diethylstilbestrol;
acolbifene, danazol, deslorelin, epitiostanol, orteronel, enzalutamide^{1,3};

### Aromatase Inhibitors

such as aminoglutethimide, anastrozole, exemestane, fadrozole, letrozole, testolactone;
formestane;

### Small Molecule Kinase Inhibitors

such as crizotinib, dasatinib, erlotinib, imatinib, lapatinib, nilotinib, pazopanib, regorafenib, ruxolitinib, sorafenib, sunitinib, vandetanib, vemurafenib, bosutinib, gefitinib, axitinib;
afatinib, alisertib, dabrafenib, dacomitinib, dinaciclib, dovitinib, enzastaurin, nintedanib, lenvatinib, linifanib, linsitinib, masitinib, midostaurin, motesanib, neratinib, orantinib, perifosine, ponatinib, radotinib, rigosertib, tipifarnib, tivantinib, tivozanib, trametinib, pimasertib, brivanib alaninate, cediranib, apatinib⁴, cabozantinib S-malate^{1,3}, ibrutinib^{1,3}, icotinib⁴, buparlisib², cipatinib⁴, cobimetinib^{1,3}, idelalisib^{1,3}, fedratinib¹, XL-647⁴;

### Photosensitizers

such as methoxsalen³;
porfimer sodium, talaporfin, temoporfin;

### Antibodies

such as alemtuzumab, besilesomab, brentuximab vedotin, cetuximab, denosumab, ipilimumab, ofatumumab, panitumumab, rituximab, tositumomab, trastuzumab, bevacizumab, pertuzumab^{2,3;}
catumaxomab, elotuzumab, epratuzumab, farletuzumab, mogamulizumab, necitumumab, nimotuzumab, obinutuzumab, ocaratuzumab, oregovomab, ramucirumab, rilotumumab, siltuximab, tocilizumab, zalutumumab, zanolimumab, matuzumab, dalotuzumab^{1,2,3}, onartuzumab^{1,3}, racotumomab¹, tabalumab^{1,3}, EMD-525797⁴, nivolumab^{1,3};

### Cytokines

such as aldesleukin, interferon alfa², interferon alfa2a³, interferon alfa2b²,3; celmoleukin, tasonermin, teceleukin, oprelvekin^{1,3}, recombinant interferon beta-1a⁴;

### Drug Conjugates

such as denileukin diftitox, ibritumomab tiuxetan, iobenguane I123, prednimustine, trastuzumab emtansine, estramustine, gemtuzumab, ozogamicin, aflibercept;
cintredekin besudotox, edotreotide, inotuzumab ozogamicin, naptumomab estafenatox, oportuzumab monatox, technetium (99mTc) arcitumomab^{1,3} vintafolide^{1,3};

### Vaccines

such as sipuleucel³; vitespen³, emepepimut-S³, oncoVAX⁴, rindopepimut³, troVax⁴, MGN-1601⁴, MGN-1703⁴;

### Miscellaneous

alitretinoin, bexarotene, bortezomib, everolimus, ibandronic acid, imiquimod, lenalidomide, lentinan, metirosine, mifamurtide, pamidronic acid, pegaspargase, pentostatin, sipuleucel³, sizofiran, tamibarotene, temsirolimus, thalidomide, tretinoin, vismodegib, zoledronic acid, vorinostat;
celecoxib, cilengitide, entinostat, etanidazole, ganetespib, idronoxil, iniparib, ixazomib, lonidamine, nimorazole, panobinostat, peretinoin, plitidepsin, pomalidomide, procodazol, ridaforolimus, tasquinimod, telotristat, thymalfasin, tirapazamine, tosedostat, trabedersen, ubenimex, valspodar, gendicine⁴, picibanil⁴, reolysin⁴, retaspimycin hydrochloride^{1,3}, trebananib^{2,3}, virulizin⁴, carfilzomib^{1,3}, endostatin⁴, immucothel⁴, belinostat³, MGN-1703⁴; ¹ Prop. INN (Proposed International Nonproprietary Name)² Rec. INN (Recommended International Nonproprietary Names)³ USAN (United States Adopted Name)⁴ no INN.

A further embodiment of the present invention is a process for the manufacture of the pharmaceutical compositions of the present invention, characterized in that one or more compounds according to the invention and one or more compounds selected from the group consisting of solid, liquid or semiliquid excipients, auxiliaries, adjuvants, diluents, carriers and pharmaceutically active agents other than the compounds according to the invention, are converted in a suitable dosage form.

In another aspect of the invention, a set or kit is provided comprising a therapeutically effective amount of at least one compound of the invention and/or at least one pharmaceutical composition as described herein and a therapeutically effective amount of at least one further pharmacologically active substance other than the compounds of the invention. It is preferred that this set or kit comprises separate packs of
a) an effective amount of a compound of formula (I), or its derivatives, prodrugs, solvates, tautomers or stereoisomers thereof as well as the physiologically acceptable salts of each of the foregoing, including mixtures thereof in all ratios, and
b) an effective amount of a further active ingredient that further active ingredient not being a compound of formula (I).

The pharmaceutical compositions of the present invention may be administered by any means that achieve their intended purpose. For example, administration may be via oral, parenteral, topical, enteral, intravenous, intramuscular, inhalant, nasal, intraarticular, intraspinal, transtracheal, transocular, subcutaneous, intraperitoneal, transdermal, or buccal routes. Alternatively, or concurrently, administration may be via the oral route. The dosage administered will be dependent upon the age, health, and weight of the recipient, kind of concurrent treatment, if any, frequency of treatment, and the nature of the effect desired. Parenteral administration is preferred. Oral administration is especially preferred.

Suitable dosage forms include, but are not limited to capsules, tablets, pellets, dragees, semi-solids, powders, granules, suppositories, ointments, creams, lotions, inhalants, injections, cataplasms, gels, tapes, eye drops, solution, syrups, aerosols, suspension, emulsion, which can be produced according to methods known in the art, for example as described below:

Tablets: mixing of active ingredient/s and auxiliaries, compression of said mixture into tablets (direct compression), optionally granulation of part of mixture before compression.

Capsules: mixing of active ingredient/s and auxiliaries to obtain a flowable powder, optionally granulating powder, filling powders/granulate into opened capsules, capping of capsules.

Semi-solids (ointments, gels, creams): dissolving/dispersing active ingredient/s in an aqueous or fatty carrier; subsequent mixing of aqueous/fatty phase with complementary fatty/aqueous phase, homogenization (creams only).

Suppositories (rectal and vaginal): dissolving/dispersing active ingredient/s in carrier material liquified by heat (rectal: carrier material normally a wax; vaginal: carrier normally a heated solution of a gelling agent), casting said mixture into suppository forms, annealing and withdrawal suppositories from the forms.

Aerosols: dispersing/dissolving active agent/s in a propellant, bottling said mixture into an atomizer.

In general, non-chemical routes for the production of pharmaceutical compositions and/or pharmaceutical preparations comprise processing steps on suitable mechanical means known in the art that transfer one or more compounds of the invention into a dosage form suitable for administration to a patient in need of such a treatment. Usually, the transfer of one or more compounds of the invention into such a dosage form comprises the addition of one or more compounds, selected from the group consisting of carriers, excipients, auxiliaries and pharmaceutical active ingredients other than the compounds of the invention. Suitable processing steps include, but are not limited to combining, milling, mixing, granulating, dissolving, dispersing, homogenizing, casting and/or compressing the respective active and non-active ingredients. Mechanical means for performing said processing steps are known in the art, for example from Ullmann's Encyclopedia of Industrial Chemistry, 5th Edition. In this respect, active ingredients are preferably at least one compound of the invention and optionally one or more additional compounds other than the compounds of the invention, which show valuable pharmaceutical properties, preferably those pharmaceutical active agents other than the compounds of the invention, which are disclosed herein.

Particularly suitable for oral use are tablets, pills, coated tablets, capsules, powders, granules, syrups, juices or drops, suitable for rectal use are suppositories, suitable for parenteral use are solutions, preferably oil-based or aqueous solutions, furthermore suspensions, emulsions or implants, and suitable for topical use are ointments, creams or powders. The compounds of the invention may also be lyophilised and the resultant lyophilisates used, for example, for the preparation of injection preparations. The preparations indicated may be sterilised and/or comprise assistants, such as lubricants, preservatives, stabilisers and/or wetting agents, emulsifiers, salts for modifying the osmotic pressure, buffer substances, dyes, flavours and/or a plurality of further active ingredients, for example one or more vitamins.

Suitable excipients are organic or inorganic substances, which are suitable for enteral (for example oral), parenteral or topical administration and do not react with the compounds of the invention, for example water, vegetable oils, benzyl alcohols, alkylene glycols, polyethylene glycols, glycerol triacetate, gelatine, carbohydrates, such as lactose, sucrose, mannitol, sorbitol or starch (maize starch, wheat starch, rice starch, potato starch), cellulose preparations and/or calcium phosphates, for example tricalcium phosphate or calcium hydrogen phosphate, magnesium stearate, talc, gelatine, tragacanth, methyl cellulose, hydroxypropylmethylcellulose, sodium carboxymethylcellulose, polyvinyl pyrrolidone and/or vaseline.

If desired, disintegrating agents may be added such as the above-mentioned starches and also carboxymethyl-starch, cross-linked polyvinyl pyrrolidone, agar, or alginic acid or a salt thereof, such as sodium alginate. Auxiliaries include, without limitation, flow-regulating agents and lubricants, for example, silica, talc, stearic acid or salts thereof, such as magnesium stearate or calcium stearate, and/or polyethylene glycol. Dragee cores are provided with suitable coatings, which, if desired, are resistant to gastric juices. For this purpose, concentrated saccharide solutions may be used, which may optionally contain gum arabic, talc, polyvinyl pyrrolidone, polyethylene glycol and/or titanium dioxide, lacquer solutions and suitable organic solvents or solvent mixtures. In order to produce coatings resistant to gastric juices or to provide a dosage form affording the advantage of prolonged action, the tablet, dragee or pill can comprise an inner dosage and an outer dosage component the latter being in the form of an envelope over the former. The two components can be separated by an enteric layer, which serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, acetyl alcohol, solutions of suitable cellulose preparations such as acetyl-cellulose phthalate, cellulose acetate or hydroxypropylmethylcellulose phthalate, are used. Dye stuffs or pigments may be added to the tablets or dragee coatings, for example, for identification or in order to characterize combinations of active compound doses.

Suitable carrier substances are organic or inorganic substances which are suitable for enteral (e.g. oral) or parenteral administration or topical application and do not react with the novel compounds, for example water, vegetable oils, benzyl alcohols, polyethylene glycols, gelatin, carbohydrates such as lactose or starch, magnesium stearate, talc and petroleum jelly. In particular, tablets, coated tablets, capsules, syrups, suspensions, drops or suppositories are used for enteral administration, solutions, preferably oily or aqueous solutions, furthermore suspensions, emulsions or implants, are used for parenteral administration, and ointments, creams or powders are used for topical application. The compounds of the invention can also be lyophilized and the lyophilizates obtained can be used, for example, for the production of injection preparations.

Other pharmaceutical preparations, which can be used orally include push-fit capsules made of gelatine, as well as soft, sealed capsules made of gelatine and a plasticizer such as glycerol or sorbitol. The push-fit capsules can contain the active compounds in the form of granules, which may be mixed with fillers such as lactose, binders such as starches, and/or lubricants such as talc or magnesium stearate and, optionally, stabilizers. In soft capsules, the active compounds are preferably dissolved or suspended in suitable liquids, such as fatty oils, or liquid paraffin. In addition, stabilizers may be added.

The liquid forms in which the novel compositions of the present invention may be incorporated for administration orally include aqueous solutions, suitably flavoured syrups, aqueous or oil suspensions, and flavoured emulsions with edible oils such as cottonseed oil, sesame oil, coconut oil or peanut oil, as well as elixirs and similar pharmaceutical vehicles. Suitable dispersing or suspending agents for aqueous suspensions include synthetic and natural gums such as tragacanth, acacia, alginate, dextran, sodium carboxymethylcellulose, methylcellulose, polyvinyl-pyrrolidone or gelatine.

Suitable formulations for parenteral administration include aqueous solutions of the active compounds in water-soluble form, for example, water-soluble salts and alkaline solutions. In addition, suspensions of the active compounds as appropriate oily injection suspensions may be administered. Suitable lipophilic solvents or vehicles include fatty oils, for example, sesame oil, or synthetic fatty acid esters, for example, ethyl oleate or triglycerides or polyethylene glycol-400 (the compounds are soluble in PEG-400).

Aqueous injection suspensions may contain substances, which increase the viscosity of the suspension, including, for example, sodium carboxymethyl cellulose, sorbitol, and/or dextran, optionally, the suspension may also contain stabilizers.

For administration as an inhalation spray, it is possible to use sprays in which the active ingredient is either dissolved or suspended in a propellant gas or propellant gas mixture (for example CO₂ or chlorofluorocarbons). The active ingredient is advantageously used here in micronized form, in which case one or more additional physiologically acceptable solvents may be present, for example ethanol. Inhalation solutions can be administered with the aid of conventional inhalers.

Possible pharmaceutical preparations, which can be used rectally include, for example, suppositories, which consist of a combination of one or more of the active compounds with a suppository base. Suitable suppository bases are, for example, natural or synthetic triglycerides, or paraffin hydrocarbons. In addition, it is also possible to use gelatine rectal capsules, which consist of a combination of the active compounds with a base. Possible base materials include, for example, liquid triglycerides, polyethylene glycols, or paraffin hydrocarbons.

For use in medicine, the compounds of the present invention may be in the form of pharmaceutically acceptable salts. Other salts may, however, be useful in the preparation of the compounds of the invention or of their pharmaceutically acceptable salts. Suitable pharmaceutically acceptable salts of the compounds of this invention are those described hereinbefore and include acid addition salts which may, for example be formed by mixing a solution of the compound according to the invention with a solution of a pharmaceutically acceptable acid such as hydrochloric acid, sulphuric acid, methanesulphonic acid, fumaric acid, maleic acid, succinic acid, acetic acid, benzoic acid, oxalic acid, citric acid, tartaric acid, carbonic acid or phosphoric acid. Furthermore, where the compounds of the invention carry an acidic moiety, suitable pharmaceutically acceptable salts thereof may include alkali metal salts, e.g. sodium or potassium salts; alkaline earth metal salts, e.g. calcium or magnesium salts; and salts formed with suitable organic bases, e.g. quaternary ammonium salts.

The pharmaceutical preparations can be employed as medicaments in human and veterinary medicine. As used herein, the term “effective amount” means that amount of a drug or pharmaceutical agent that will elicit the biological or medical response of a tissue, system, animal or human that is being sought, for instance, by a researcher or clinician. Furthermore, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function. Said therapeutic effective amount of one or more of the compounds of the invention is known to the skilled artisan or can be easily determined by standard methods known in the art.

The compounds of the present invention and the optional additional active substances are generally administered analogously to commercial preparations. Usually, suitable doses that are therapeutically effective lie in the range between 0.0005 mg and 1000 mg, preferably between 0.005 mg and 500 mg and especially between 0.5 mg and 100 mg per dose unit. The daily dose is preferably between about 0.001 mg/kg and 10 mg/kg of body weight.

Those of skill will readily appreciate that dose levels can vary as a function of the specific compound, the severity of the symptoms and the susceptibility of the subject to side effects. Some of the specific compounds are more potent than others. Preferred dosages for a given compound are readily determinable by those of skill in the art by a variety of means. A preferred means is to measure the physiological potency of a given compound.

The specific dose for the individual patient, in particular for the individual human patient, depends, however, on the multitude of factors, for example on the efficacy of the specific compounds employed, on the age, body weight, general state of health, the sex, the kind of diet, on the time and route of administration, on the excretion rate, the kind of administration and the dosage form to be administered, the pharmaceutical combination and severity of the particular disorder to which the therapy relates. The specific therapeutic effective dose for the individual patient can readily be determined by routine experimentation, for example by the doctor or physician, which advises or attends the therapeutic treatment.

The compounds of the present invention can be prepared according to the procedures of the following Schemes and Examples, using appropriate materials, and are further exemplified by the following specific examples. They may also be prepared by methods known per se, as described in the literature (for example in standard works, such as Houben-Weyl, Methoden der Organischen Chemie [Methods of Organic Chemistry], Georg Thieme Verlag, Stuttgart; Organic Reactions, John Wiley & Sons, Inc., New York), to be precise under reaction conditions which are known and suitable for the said reactions. Use can also be made of variants which are known per se, but are not mentioned here in greater detail.

Likewise, the starting materials for the preparation of compounds of the present invention can be prepared by methods as described in the examples or by methods known per se, as described in the literature of synthetic organic chemistry and known to the skilled person, or can be obtained commercially. The starting materials for the processes claimed and/or utilized may, if desired, also be formed in situ by not isolating them from the reaction mixture, but instead immediately converting them further into the compounds of the invention or intermediate compounds. On the other hand, in general it is possible to carry out the reaction stepwise.

Preferably, the reaction of the compounds is carried out in the presence of a suitable solvent, which is preferably inert under the respective reaction conditions. Examples of suitable solvents comprise but are not limited to hydrocarbons, such as hexane, petroleum ether, benzene, toluene or xylene; chlorinated hydrocarbons, such as trichlorethylene, 1,2-dichloroethane, tetrachloromethane, chloroform or dichloromethane; alcohols, such as methanol, ethanol, isopropanol, n-propanol, n-butanol or tert-butanol; ethers, such as diethyl ether, diisopropyl ether, tetrahydrofuran (THF) or dioxane; glycol ethers, such as ethylene glycol monomethyl or monoethyl ether or ethylene glycol dimethyl ether (diglyme); ketones, such as acetone or butanone; amides, such as acetamide, dimethylacetamide, dimethylformamide (DMF) or N-methyl pyrrolidinone (NMP); nitriles, such as acetonitrile; sulfoxides, such as dimethyl sulfoxide (DMSO); nitro compounds, such as nitromethane or nitrobenzene; esters, such as ethyl acetate, or mixtures of the said solvents or mixtures with water.

The reaction temperature is between about −100° C. and 300° C., depending on the reaction step and the conditions used.

Reaction times are generally in the range between a fraction of a minute and several days, depending on the reactivity of the respective compounds and the respective reaction conditions. Suitable reaction times are readily determinable by methods known in the art, for example reaction monitoring. Based on the reaction temperatures given above, suitable reaction times generally lie in the range between 10 minutes and 48 hours.

Moreover, by utilizing the procedures described herein, in conjunction with ordinary skills in the art, additional compounds of the present invention claimed herein can be readily prepared. The compounds illustrated in the examples are not, however, to be construed as forming the only genus that is considered as the invention. The examples further illustrate details for the preparation of the compounds of the present invention. Those skilled in the art will readily understand that known variations of the conditions and processes of the following preparative procedures can be used to prepare these compounds.

The present invention also refers to a process for manufacturing a compound according to formula (I), or derivatives, N-oxides, prodrugs, solvates, tautomers or stereoisomers thereof as well as the physiologically acceptable salts of each of the foregoing. This process is characterized in that
(a) a compound of formula (II)

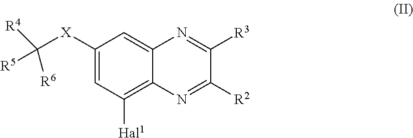

wherein
Hal¹ denotes Cl, Br or I;
R², R³, R⁴, R⁵, R⁶, X have the same meaning as defined hereinabove and in claims 1 to 31 for compounds of formula (I);
is reacted under C-C coupling reaction conditions which conditions may utilize one or more suitable C-C coupling reaction reagents including catalysts
with a compound R¹—RG^{a}
wherein
R¹ have the same meaning as defined hereinabove and in claims 1 to 31 for compounds of formula (I);
RG^{a} denotes a chemical moiety being reactive under the particular C-C coupling reaction conditions utilized;
or
(b) a compound of formula (III)

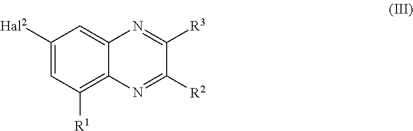

wherein
Hal² denotes Cl, Br or I;
R¹, R², R³ have the same meaning as defined hereinabove and in claims 1 to 31 for compounds of formula (I);
is reacted under C—N coupling reaction conditions which conditions may utilize one or more suitable C—N coupling reaction reagents including catalysts
with a compound R⁴R⁵R⁶C—NHR⁷
wherein
R⁴, R⁵, R⁶, R⁷ have the same meaning as defined hereinabove and in claims 1 to 31 for compounds of formula (I);
or
(c) a compound of formula (III)

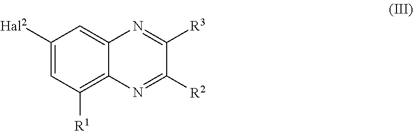

wherein
Hal² denotes Cl, Br or I;
R¹, R², R³ have the same meaning as defined hereinabove and in claims 1 to 31 for compounds of formula (I);
is reacted under C—O coupling reaction conditions which conditions may utilize one or more suitable C—O coupling reaction reagents including catalysts
with a compound R⁴R⁵R⁶C—OH
wherein
X denotes O;
R⁴, R⁵, R⁶ have the same meaning as defined hereinabove and in claims 1 to 31 for compounds of formula (I).

As will be understood by the person skilled in the art of organic synthesis compounds of the present invention, in particular compounds of formula (I), are readily accessible by various synthetic routes, some of which are exemplified in the accompanying Experimental Part. The skilled artisan will easily recognize which kind of reagents and reactions conditions are to be used and how they are to be applied and adapted in any particular instance—wherever necessary or useful—in order to obtain the compounds of the present invention. Furthermore, some of the compounds of the present invention can readily be synthesized by reacting other compounds of the present invention under suitable conditions, for instance, by converting one particular functional group being present in a compound of the present invention, or a suitable precursor molecule thereof, into another one by applying standard synthetic methods, like reduction, oxidation, addition or substitution reactions; those methods are well known to the skilled person. Likewise, the skilled artisan will apply—whenever necessary or useful—synthetic protecting groups; suitable protecting groups as well as methods for introducing and removing them are well-known to the person skilled in the art of chemical synthesis and are described, in more detail, in, e.g., P.G.M. Wuts, T.W. Greene, “Greene's Protective Groups in Organic Synthesis”, 4th edition (2006) (John Wiley & Sons).

A particularly versatile starting point for making compounds of formula (I) are 5-bromo-7-chloroquinoxaline (Int 2) and 7-bromo-5-chloroquinoxaline (Int 3) both of which are readily available by applying in analogy synthetic methods described in WO 2010/20363 A1.

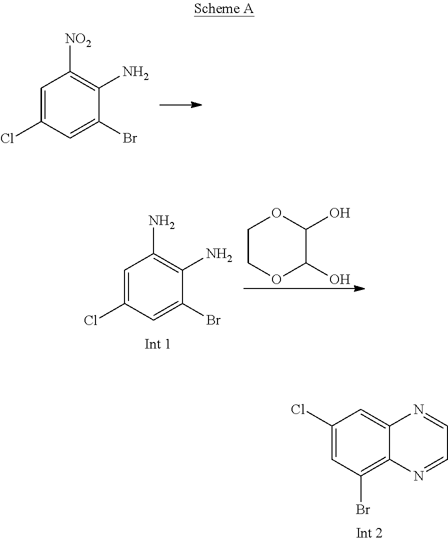

2-Bromo-4-chloro-6-nitrophenylamine is converted into 3-bromo-5-chlorobenzene-2-diamine (Int 1) by utilizing suitable reduction means, e.g., tin(II)-chloride, which in turn is converted into 5-bromo-7-chloroquinoxaline (Int 2) by reacting it with 2,3-dihydroxy-1,4-dioxane.

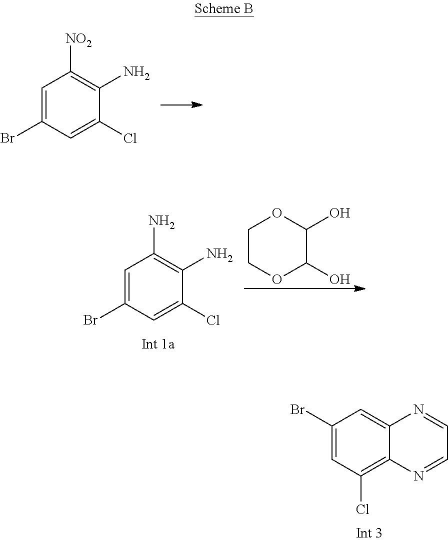

Likewise, 7-bromo-5-chloroquinoxaline (Int 3) is available by applying the same methodology under similar conditions (see Scheme B). It is to be noted that compounds of formula (I) in which either one or both substituents R² and R³ do not denote hydrogen, are available from precursor molecules similar to Int 2 and Int 3 by applying similar methods and optional purification/separation from isomers (see Scheme C):

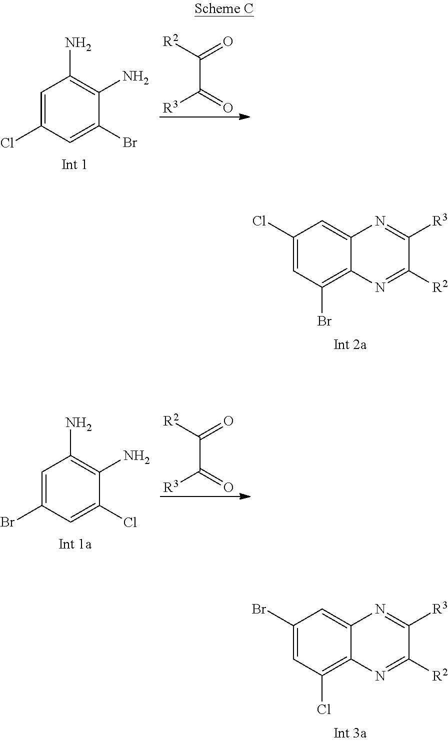

In one particular approach for making compounds of the present invention precursor molecule Int 2 (or Int 2a, as the case may be) is converted into a compound of formula (III) with Hal² being bromine and R¹ being defined as in the description hereinabove and in the claims by applying either C—C coupling reaction conditions (if R¹ is connected to the quinoxaline system via a carbon atom) or C—N coupling reaction conditions (if R¹ is connected to the quinoxaline system via a nitrogen atom).

Typical suitable C-C coupling reactions are, among others, the Heck reaction, the Suzuki coupling, the Stille coupling, the Negishi coupling and coupling reactions utilizing organo cuprates, and well-known variants thereof. Depending on the specific method applied reagents, solvents and reaction conditions are selected accordingly. For instance, in case the introduction of R¹ is performed by utilizing Suzuki coupling conditions, precursor molecule Int 2 (or Int 2a) may be reacted with a suitable borate or boronate ester (B(OSub)₃, with Sub being a suitable substituent, radical or residue) (like trimethylborate or 4,4,5,5-tetramethyl-2-(tetramethyl-1,3,2-dioxaborolan-2-yl)-1,3,2-dioxaborolane) in the presence of an organometallic palladium (II) catalyst (like [1,1′-bis(diphenyl)phosphino)ferrocene]-dichloropalladium(II) dichloromethane complex) and optionally potassium acetate in order to form a derivative of Int 2 (or Int 2a) in which the bromine substituent is replaced by —B(OH)₂ or —B(OSub)₂, as the case may be; this derivative may then be reacted with a suitable halide R¹-Hal in the presence of a palladium(0) complex (e.g., tetrakis(triphenylphosphine)palladium(0)) and a base (e.g., sodium, potassium or cesium carbonate) to build a compound of formula (III). Similarly, the same compound of formula (III) can be obtained by forming a boron-substituted precursor R¹—B(OH)₂ or R¹—B(OSub)₂ and reacting it with Int 2 (or Int 2a) under similar conditions.

Likewise, C—N coupling reactions may be any suitable C—N coupling reaction of a heterocyclic system or a molecule bearing a reactive amino group with precursor molecule Int 2 (or Int 2a). Depending on the specific coupling reaction applied, it may well be that one or both of the reaction partners are subject to chemical transformation into intermediates before the reaction with the appropriate reaction partner occurs; for instance, the suitably substituted halide may be transformed into a respective boronic acid or boronic acid ester derivative before the reaction with the heterocyclic system or the reactive amine derivative occurs. Preferably, this coupling reaction is performed in the presence of a transition metal catalyst. Well-known examples of such C—N coupling reactions are, among others, the Hartwig-Buchwald reaction, the Ullmann coupling reaction, reactions similar to Suzuki or Heck reaction and coupling reactions utilizing organo cuprates. Depending on the specific method applied reagents, solvents and reaction conditions are selected accordingly.

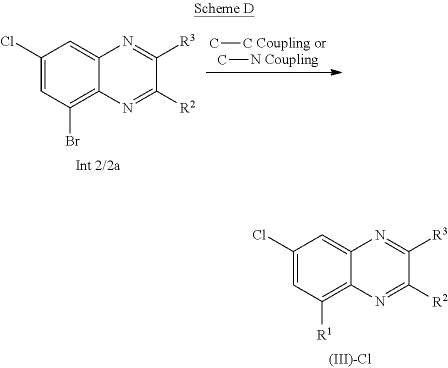

In order to obtain various compounds of formula (I) compounds of formula (III)-Cl obtained as shown in Scheme D may then be subjected to further synthetic modifications for introducing suitable functional groups that allow for, if required, still further modifications. One of these various methods is depicted in Scheme E showing the conversion of a compound of formula (III)-Cl into a compound of formula (IV)-NH2, i.e., of a chloride into an amine, which may then be subjected to further reactions.

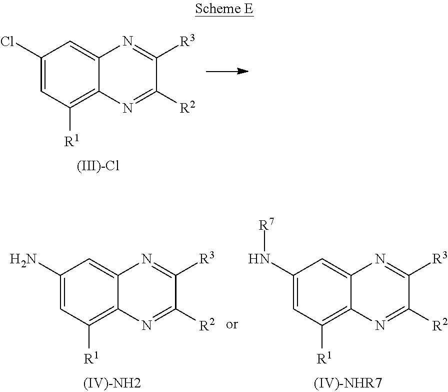

This functional group conversion to the amine (IV)-NH2 may be achieved by subjecting the chloride (III)-Cl to a Hartwig-Buchwald reaction, i.e., by reacting it with ammonia (or an ammonia solution) in the presence of a palladium(II) catalyst, a suitable phosphine ligand and sodium tert.-butylate (e.g., Pd₂(dba)₃/Me₄tBuXPhos/NaOtBu/NH₃). If an amine R⁷—NH₂ (with R⁷ being as defined in the specification herein or in the claims and not being hydrogen) is used instead of ammonia (which could also be denoted as R⁷—NH₂ with R⁷ being H), compounds of formula (IV)-NHR7 may be obtained.

Compounds of formula (IV)-NH2 or (IV)-NHR7 may be the starting point for obtaining compounds of formula (I) with X being N—R⁷ (with R⁷ being as defined in the specification hereinabove or in the claims). For instance, compounds of the present invention of formula (I) with R⁵ and R⁶ forming together a C═CHR^{D4} moiety can readily be obtained by reacting the amine (IV)-NH2 with a suitably substituted ketone as shown in Scheme F; the resulting olefinic double bond may optionally be converted into an aliphatic C—C single bond by utilizing a suitable reductions means, e.g. NaBH(OAc)₃. Optional nucleophilic substitution then yields compounds of formula (I) with X being N—R⁷ wherein R⁷ is not hydrogen. Alternatively, these latter compounds may be obtained by utilizing a compound of formula (IV)-NHR7 as starting material.

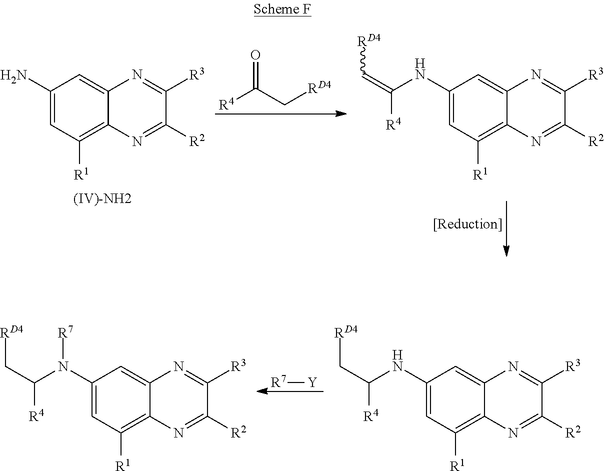

Compounds of formula (IV)-NH2 may also be the starting point for the formation of compounds of the present invention with X being NR⁷ and R⁵ and R⁶ both being hydrogen; the compound (IV)-NH2 may be reacted with a suitably substituted aldehyde, followed by reduction and optional introduction of a moiety R⁷ being different than H (Scheme G). Again, in some instances these compounds of formula (I) may be obtained by utilizing a compound of formula (IV)-NHR7 instead of formula (IV)-NH2 as starting material for the reaction with aldehyde R⁴—CHO and subsequent reduction:

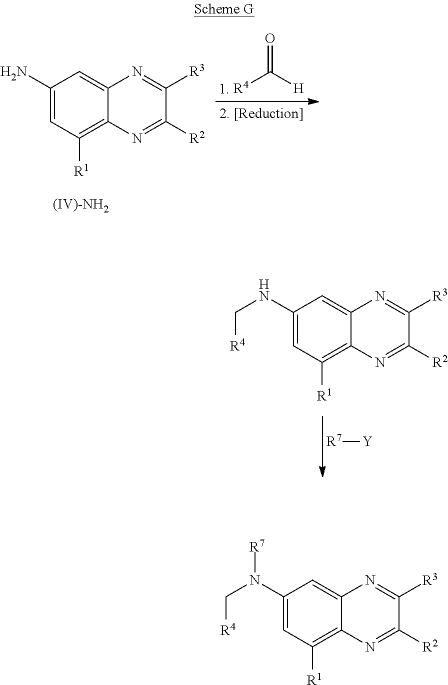

This methodology may be particular useful for the introduction of functionalized or rather complex substituents R⁴; it can be used, inter alia, to prepare compounds of formula (I) in which R⁴ denotes Ar^{X}, Ar^{X}—Ar^{Y}, Ar^{X}-Hetar^{Y}, Ar^{X}-Hetcyc^{Y}, Ar^{X}-LA^{Z}-Ar^{Y}, Ar^{X}-LA^{Z}-Hetar^{Y}, Ar^{X}-LA^{Z}-Hetcyc^{Y}, Hetar^{X}, Hetar^{X}-Ar^{Y}, Hetar^{X}-Hetar^{Y}, Hetar^{X}-Hetcyc^{Y}, Hetar^{X}-LA^{Z}-Ar^{Y}, Hetar^{X}-LA^{Z}-Hetar^{Y}, Hetar^{X}-LA^{Z}-Hetcyc^{Y}, Hetcyc^{X}, Hetcyc^{X}-Ar^{Y}, Hetcyc^{X}-Hetar^{Y}, Hetcyc^{X}-Hetcyc^{Y}, Hetcyc^{X}-LA^{Z}-Ar^{Y}, Hetcyc^{X}-LA^{Z}-Hetar^{Y}, Hetcyc^{X}-LA^{Z}-Hetcyc^{Y}, LA^{X}, LA^{Z}-Ar^{Y}, LA^{Z}-Hetar^{Y}, LA^{Z}-Hetcyc^{Y}, CA^{X}, and optionally bear a halogen substituent Hal. Depending on the very nature of R⁴, it may be introduced directly by reacting a compound of formula (IV)-NH2 or (IV)-NHR7 with a suitably substituted aldehyde R⁴—CHO; in some instances it may be preferable or even necessary to build up a particular substituent in stepwise manner. This approach is exemplified in Scheme H and can easily be adapted to different substitution pattern, e.g., where Ar^{X} is replaced by, for instance, Hetar^{X}, Hetcyc^{X} or CA^{X}.

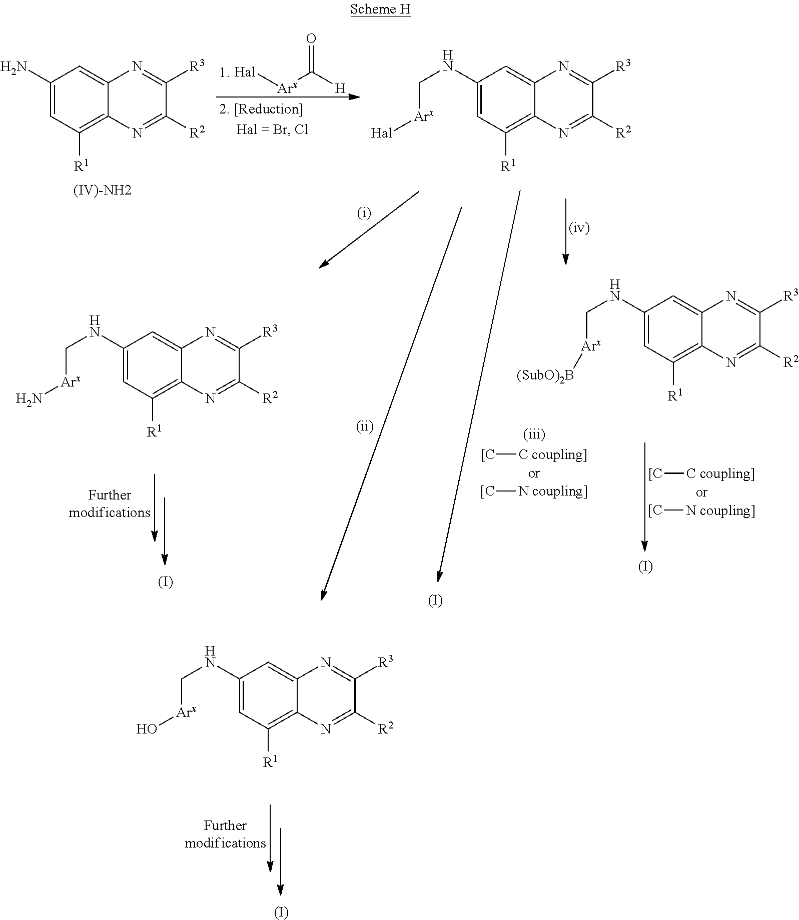

Similar to the conversion depicted in Scheme E, the halogen functional group can be converted to the respective amino group (see route (i)) by subjecting the halogen compound to a Hartwig-Buchwald reaction, i.e., by reacting it with ammonia in the presence of a palladium(II) catalyst, a suitable phosphine ligand and sodium tert-butylate (e.g., Pd₂(dba)₃/Me₄tBuXPhos/NaOtBu/NH₃). The amine thus obtained can subsequently be converted into other compounds of the present invention of formula (I). The conversion of the halogen functional group into a hydroxyl functional group (see route (ii) in Scheme H) can be effected, for instance, by applying a palladium(II) catalyst in the presence of a suitable phosphine and potassium hydroxide. Again, the hydroxyl-substituted compound thus obtained can subsequently be converted into other compounds of the present invention of formula (I).

According to reaction route (iii) of Scheme H, utilizing well-known C—C coupling or C—N coupling reactions yields still further compounds of the present invention. Typical suitable C-C coupling reactions that can be applied are, among others, the Heck reaction, the Suzuki coupling, the Stille coupling, the Negishi coupling and coupling reactions utilizing organo cuprates, and well-known variants thereof. Depending on the specific method applied reagents, solvents and reaction conditions are selected accordingly. For instance, in case the introduction of a Hetar^{Y} residue is performed by utilizing Suzuki coupling conditions, the halogen-substituted compound depicted in Scheme H may be reacted with a suitable Hetar^{Y} boronate (Hetar^{Y}-B(OH)₂ or Hetar^{Y}-B(OSub)₂ (with Sub being a suitable substituent)) in the presence of an organometallic palladium (II) catalyst (like [1,1′-bis(diphenyl)phosphino)ferrocene]-dichloropalladiun(II) dichloromethane complex) and optionally potassium acetate in order to form a compound of formula (I) in which R⁴ denotes Ar^{X}-Hetar^{Y}. Likewise, an appropriate C—N coupling reaction may be any suitable C—N coupling reaction of a heterocyclic system or a molecule bearing a reactive amino group with the halogen-substituted compound shown in Scheme H. Depending on the specific coupling reaction applied, it may well be that one or both of the reaction partners are subject to chemical transformation into intermediates before the reaction with the appropriate reaction partner occurs. Preferably, this coupling reaction is performed in the presence of a transition metal catalyst. Well-known examples of such C—N coupling reactions are, among others, the Hartwig-Buchwald reaction, the Ullmann coupling reaction, reactions similar to Suzuki or Heck reaction and coupling reactions utilizing organo cuprates. Depending on the specific method applied reagents, solvents and reaction conditions are selected accordingly.

Similar C-C couplings or C—N couplings, as the case may be, can be utilized, when synthetic approach (iv) of Scheme H is applied: Here the halogen-substituted compound of Scheme H is converted into a suitable boronic acid or boronic acid ester precursor which is then reacted, typically in the presence of a palladium(II) catalyst, an appropriate phosphine ligand and a base, with a bromine or chlorine substituted reaction partner (e.g., Ar^{Y}—Br, Hetar^{Y}-Br, Hetcyc^{Y}-Br) to afford the respective compound of formula (I).

Another approach for making compounds of the present invention of formula (I) utilizes one of the above-mentioned precursors Int 3 and Int 3a. By applying one of the C—N coupling methodologies already described in some detail hereinabove Int 3 (or Int 3a) can be converted into a compound of formula (II) with Hal¹ being Cl and X being NH (Scheme I):

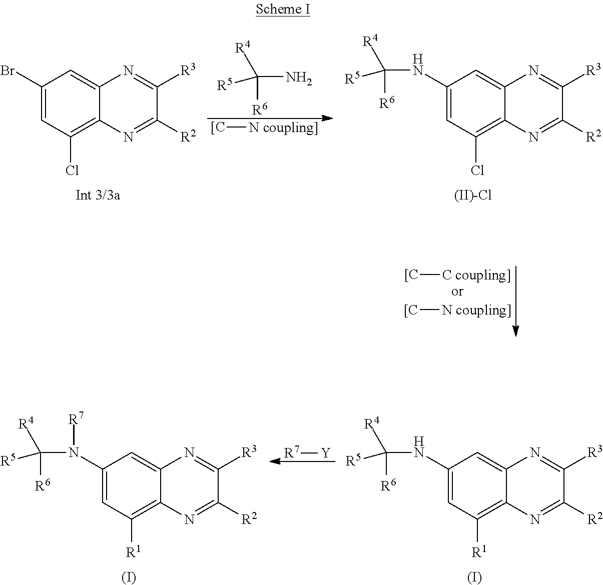

Replacing the chlorine substituent of compound (II)-Cl by substituent R¹ can then be effected by utilizing similar reaction methods already described above for making compounds of formula (III)-Cl (Scheme D), i.e. C—C coupling or C—N coupling reactions described herein. Introduction of a substituent R⁷ not being hydrogen can be effected, e.g., by nucleophilic substitution with a suitable reaction partner R⁷—Y (Y being an appropriate leaving group). Alternatively, the moiety R⁷ not being hydrogen may be introduced by utilizing a suitably substituted amine R⁴R⁵R⁶C—NHR⁷ in the C—N coupling reaction with Int 3 or Int 3a.

Compounds of formula (I) with X denoting O (oxygen) are available by the synthetic route depicted in Scheme J:

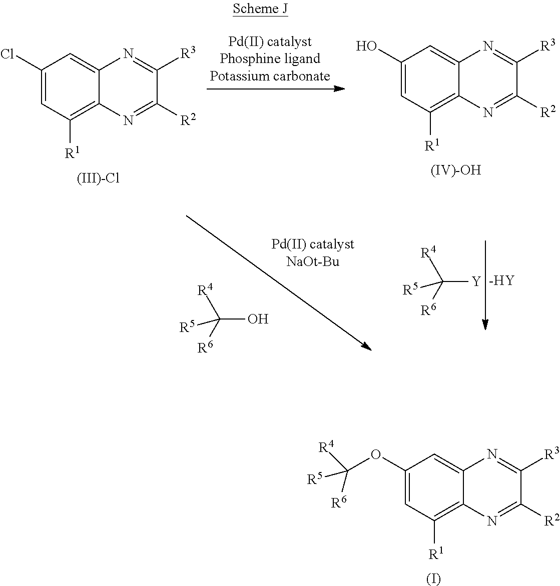

A compound of formula (III)-Cl may be converted into the respective hydroxyl-substituted compound of formula (IV)-OH by utilizing a suitable palladium(II) catalyst in the presence of an appropriate phosphine ligand and K₂CO₃. The hydroxyl compound (IV)-OH can then be reacted with a compound of formula R⁴R⁵R⁶C—Y (with Y being a typical leaving group) under conditions that are usually applied for nucleophilic substitution reactions to afford the compound of formula (I). Alternatively, a compound of formula (III)-Cl may directly be converted into the respective compound of formula (I) by reacting it with the alcohol R⁴R⁵R⁶C—OH under palladium(II)/phosphine ligand catalysis in the presence of sodium tert-butylate. This alternative route is in particular useful for making compounds of formula (I) with R⁵═R⁶═H.

The present invention also refers to a compound of formula (II) or (Ill) which are useful intermediates for making compounds of the present invention of formula (I)

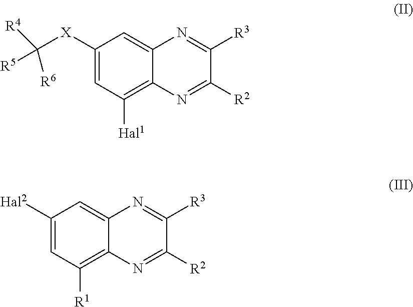

or salts thereof,
wherein
Hal¹ and Hal² denote independently from each other Cl, Br or I;
R¹, R², R³, R⁴, R⁵, R⁶, X have the same meaning as defined in claims 1 to 31 for compounds of formula (I) and hereinabove;
with the proviso that
7-chloro-5-[2-fluoro-4-methyl-5-(2,2,2-trifluoroethanesulfinyl)phenyl]-quinoxaline and
7-chloro-5-{2-fluoro-4-methyl-5-[(2,2,2-trifluoroethyl)sulfanyl]-phenyl}quinoxaline, which are both disclosed in WO 2012/176856 A2, are excluded.

### Experimental Part

### Abbreviations

Some abbreviations that may appear in this application are defined as follows hereinafter:

| Abbreviation | Meaning |
|---|---|
| ACN | Acetonitrile |
| Ac₂O | Acetic anhydride |
| AdBrettPhos Pd | [2-(Di-1-adamantylphosphino)-2′,4′,6′-triisopropyl-3,6- |
| G3 | dimethoxybiphenyl][2-(2′-amino-1,1′- |
| | biphenyl)]palladium(II) methanesulfonate |
| [(Cinnamyl)PdCl]₂ | Palladium(π-cinnamyl) chloride dimer |
| BINAP | (±)-2,2′-Bis(diphenylphosphino)-1,1′-binaphthalene |
| BippyPhos | 5-(Di-tert-butylphosphino)-1′,3′,5′-triphenyl-1′H- |
| | [1,4′]bipyrazole |
| Boc | tert-Butoxycarbonyl |
| Boc₂O | di-tert-Butyl dicarbonate (Boc anhydride) |
| BrettPhos | 2-(Dicyclohexylphosphino)3,6-dimethoxy-2′,4′,6′- |
| | triisopropyl-1,1′-biphenyl |
| BrettPhos | Chloro[2-(dicyclohexylphosphino)-3,6-dimethoxy-2′,4′, |
| precatalyst | 6′-triisopropyl-1,1′-biphenyl][2-(2- |
| | aminoethyl)phenyl]palladium(II) |
| t-BuBrettPhos | 2-(Di-tert-butylphosphino)-2′,4′,6′-triisopropyl-3,6-dimethoxy- |
| | 1,1′-biphenyl |
| n-BuOH | n-Butanol |
| t-BuOH | 2-Methylpropan-2-ol |
| tBuXPhos | 2-Di-tert-butylphosphino-2′,4′,6′-triisopropylbiphenyl |
| CH₃BrMg | Methylmagnesium bromide |
| CH₃I | Iodomethane |
| CuBr₂ | Copper(II) bromide |
| DCC | N,N′-Dicyclohexylcarbodiimide |
| DCM | Dichloromethane |
| Dess-Martin | 1,1,1-Triacetoxy-1,1-dihydro-1,2-benziodoxol-3(1H)- |
| Reagent | one |
| DIPEA | Ethyldiisopropylamine |
| DMA | N,N-Dimethylacetamide |
| DMAP | 4-(N,N-Dimethylamine)pirydyne |
| DME | 1,2-Dimethoxyethane |
| DMF | N,N-Dimethylformamide |
| DMT-MM | 2,4-dimethoxy-6-(1-methyl-1λ⁴-piperidin-1-yl)-1,3,5- |
| | triazine hydrochloride |
| DMSO | Dimethyl sulfoxide |
| dppf | Diphenylphosphinoferrocene |
| EtOAc | Ethyl acetate |
| EtOH | Ethanol |
| Et₂O | Diethyl ether |
| Hantzsch ester | Diethyl 1,4-dihydro-2,6-dimethyl-3,5- |
| | pyridinedicarboxylate |
| Herrmann's | trans-Bis(acetato)bis[o-(di-o- |
| catalyst | tolylphosphino)benzyl]dipalladium(II) |
| HPLC | High-performance liquid chromatography |
| KOAc | Potassium acetate |
| LiHMDS | Lithium bis(trimethylsilyl)amide solution |
| Me₄tBuXPhos | 2-Di-tert-butylphosphino-3,4,5,6-tetramethyl-2′,4′,6′- |
| | triisopropyl-1,1′-biphenyl |
| MeOH | Methanol |
| MnO₂ | Manganese(IV) oxide |
| MW | Microwave |
| NaOAc | Sodium acetate |
| NaBH(OAc)₃ | Sodium triacetoxyborohydride |
| NaOtBu | Sodium tert-butoxide |
| NH₄OAc | Ammonia acetate |
| Pd(dppf)Cl₂ | [1,1′-Bis(diphenylphosphino)ferrocene]dichloropalladium(II) |
| Pd(dppf)Cl₂—CH₂Cl₂ | [1,1′- |
| | Bis(diphenylphosphino)ferrocene]dichloropalladium(II), complex |
| | with dichloromethane |
| Pd(OAc)₂ | Palladium(II) acetate |
| Pd(PPh₃)₄ | Tetrakis(triphenylphosphine)palladium(0) |
| Pd₂(dba)₃ | Tris(dibenzylideneacetone)dipalladium(0) |
| PTSA | p-Toluenesulfonic acid monohydrate |
| RM | reaction mixture |
| rt | room temperature |
| TBN | tert-Butyl nitrite |
| t-BuBrettPhos | 2-(Di-tert-butylphosphino)-2′,4′,6′-triisopropyl-3,6- |
| | dimethoxy-1,1′-biphenyl |
| TEA | Triethylamine |
| TEA*HCl | Triethylamine hydrochloride |
| TFA | Trifluoroacetic acid |
| TFAA | Trifluoroacetic acid anhydride |
| THF | Tetrahydrofuran |
| Ti(OEt)₄ | Titanium(IV) butoxide |
| TMCS | Chlorotrimethylsilane |
| TTIP | Titanium(IV) isopropoxide |
| Trityl-Cl | Chlorotriphenylmethane |
| Trityl | Triphenylmethane |
| Xantphos | 4,5-Bis(diphenylphosphino)-9,9-dimethylxanthene |
| Xphos | 2-Dicyclohexylphosphino-2′,4′,6′-triisopropylbiphenyl |

The compounds of the present invention can be prepared according to the procedures of the following Schemes and Examples, using appropriate materials and are further exemplified by the following specific examples. Analytical data of compounds made according to the following examples are shown in Table 1.

The invention will be illustrated, but not limited, by reference to the specific embodiments described in the following examples. Unless otherwise indicated in the schemes, the variables have the same meaning as described above and in the claims.

Unless otherwise specified, all starting materials are obtained from commercial suppliers and used without further purifications. Unless otherwise specified, all temperatures are expressed in ° C. and all reactions are conducted at rt. Compounds are purified by either silica chromatography or preparative HPLC.

¹H NMR:

¹H NMR is recorded on 400 MHz spectrometers. Chemical shifts (6) are reported in ppm relative to the residual solvent signal (δ=2.5 ppm for ¹H NMR in DMSO-d6). ¹H NMR data are reported as follows: chemical shift (multiplicity, coupling constants and number of hydrogens). Multiplicity is abbreviated as follows: s (singlet), d (doublet), t (triplet), q (quartet), m (multiplet), br (broad).

NMR, UPLC, HPLC and MS data provided in the examples described below are registered on:

NMR: Bruker Avance III HD 400 MHz, probe BBO

UHPLC-MS
Shimadzu LC-MS 2020
HPLC with UV-Vis or DAD detector
Column: Waters Acquity UPLC HSS C18, 50 mm×2.1 mm×1.8 μm

HPLC-MS:
DIONEX ULTIMATE 3000
Bruker HCT ION TRAP

Methods:

Shimadzu

Equipment:
UHPLC with UV-Vis detector
column: Waters Acquity UPLC HSS C18, 2.1×50 mm 1.8 μm with guard column

Eluents:
(A) 0.1% formic acid-water solution
(B) 0.1% formic acid-ACN solution

Analytical Method:

Autosampler:
injection volume: 1 μL

Pump:
flow: 0.5 mL/min

| Time [min] | [%] A | [%] B |
|---|---|---|
| 0.0 | 95 | 5 |
| 4.0 | 5 | 95 |
| 5.0 | 5 | 95 |
| 5.2 | 95 | 5 |
| 6.0 | 95 | 5 |

Column Compartment:
column temperature: 25° C.
time of analysis: 6.0 min

Detector:
wavelength: 214 nm, 254 nm, 280 nm

MS: Single Quadrupole

Ionization method: ESI

DL temperature: 230° C.

Heat block temperature: 230° C.

Drying gas flow: 10.0 L/min

Positive Ion Polarity

Scan range: 100-1000 m/z

Rot-C18-1

Equipment:
HPLC with UV-Vis or DAD detector
column: Waters Symmetry C18 3.9×150 mm 5 μm

Eluents:
(A) 0.1% formic acid-water solution
(B) 0.1% formic acid-ACN solution

Analytical Method:

Autosampler:
injection volume: 3 μL

Pump:
flow: 1.0 mL/min

| Time [min] | [%] A | [%] B |
|---|---|---|
| 0.0 | 95 | 5 |
| 5.0 | 95 | 5 |
| 25.0 | 20 | 80 |
| 27.0 | 20 | 80 |
| 28.0 | 95 | 5 |
| 30.0 | 95 | 5 |

Column Compartment:
column temperature: 25° C.
time of analysis: 30 min

Detector:
DAD

MS: HCT

Drying gas temperature: 365° C.

Drying gas flow: 9.0 L/min

Nebulization gas pressure: 40 psi

Positive Ion Polarity

Scan range: 100-1000 m/z

BCM-30

Equipment:
HPLC with UV-Vis or DAD detector
column: Waters Symmetry C18 3.9×150 mm 5 μm

Eluents:
(A) 0.1% formic acid-water solution
(B) 0.1% formic acid-ACN solution

Analytical Method:

Autosampler:
injection volume: 3 μL

Pump:
flow: 1.2 mL/min

| Time [min] | [%] B |
|---|---|
| 0.0 | 20 |
| 20.0 | 80 |
| 22.0 | 80 |
| 22.5 | 95 |
| 25.0 | 95 |
| 25.3 | 20 |
| 30.0 | 20 |

Column Compartment:
column temperature: 25° C.
time of analysis: 30 min

Detector:
wavelength: 200 nm

### SYNTHETIC EXAMPLES

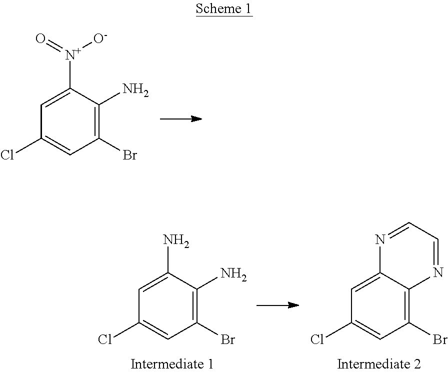 [SMILES: Nc1c(Br)cc(Cl)cc1[N+](=O)[O-] ; Nc1cc(Cl)cc(Br)c1N ; Clc1cc(Br)c2nccnc2c1]

### Intermediate 1 (see US2013/116262 A1)

### 3-Bromo-5-chlorobenzene-1,2-diamine

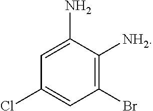

To a stirred solution of tin(II)chloride dihydrate (53.8 g; 238 mmol; 6.00 eq.) in EtOAc (400 mL), 2-bromo-4-chloro-6-nitrophenylamine (10 g; 39.8 mmol; 1.0 eq.) is added in three portions. The reaction is refluxed for 2 h. After this time, the solvent is evaporated and dry residue is suspended in DCM (1 L) and then aqueous solution of NaOH is added (˜300 mL, 10 M, >50 eq.). All reagents are stirred for 4 h and after this time, an organic layer is separated, washed with water and brine and dried over anhydrous Na₂SO₄. Drying agent is filtered off and solvent is evaporated under reduced pressure. 3-Bromo-5-chlorobenzene-1,2-diamine (Intermediate 1) (8.4 g; yield 95%; 97% by UPLC) is obtained as a beige solid and used in the next step without further purification.

### Intermediate 2 (cf. WO2010/20363 A1)

### 5-bromo-7-chloroquinoxaline

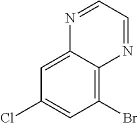 [SMILES: Clc1cc(Br)c2nccnc2c1]

3-bromo-5-chloro-1,2-diaminobenzene Intermediate 1 (8.4 g; 37.9 mmol; 1.0 eq.) is dissolved in EtOH (250 mL) and then 2,3-dihydroxy-1,4-dioxane (4.5 g, 37.9 mmol; 1.0 eq.) is added. The mixture is stirred for 4 h at rt and a second portion of 2,3-dihydroxy-1,4-dioxane (2.3 g; 18.9 mmol; 0.5 eq.) is added. After stirring for 24 h at rt, precipitate is filtered off, washed with EtOH and dried under vacuo to give 5-bromo-7-chloroquinoxaline (Intermediate 2) as a beige solid (6.71 g; yield 74%; 96% by UPLC).

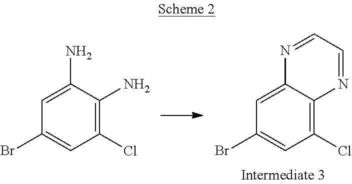 [SMILES: Nc1cc(Br)cc(Cl)c1N ; Clc1cc(Br)cc2nccnc12]

### Intermediate 3 (cf. WO2010/20363 A1)

### 7-bromo-5-chloroquinoxaline

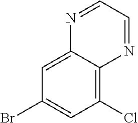 [SMILES: Clc1cc(Br)cc2nccnc12]

5-bromo-3-chloro-1,2-diaminobenzene (4.6 g; 20 mmol; 1.0 eq.) is dissolved in EtOH (200 mL) and then 2,3-dihydroxy-1,4-dioxane (2.5 g, 20 mmol; 1.0 eq.) is added. The mixture is stirred for 4 h at rt and a second portion of 2,3-dihydroxy-1,4-dioxane (1.3 g; 10 mmol; 0.5 eq.) is added. After stirring for 24 h at rt, RM is concentrated in a rotary evaporator and the residue is purified by FCC to provide 7-bromo-5-chloroquinoxaline (Intermediate 3) as a beige solid (4.7 g; yield 92%; 98% by UPLC).

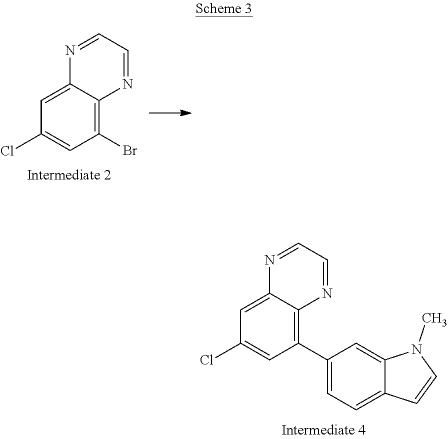 [SMILES: Clc1cc(Br)c2nccnc2c1 ; Cn1ccc2ccc(-c3cc(Cl)cc4nccnc34)cc21]

### Intermediate 4—General Procedure 1

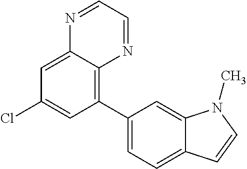 [SMILES: Cn1ccc2ccc(-c3cc(Cl)cc4nccnc34)cc21]

A sealed tube is charged with 5-bromo-7-chloroquinoxaline (Intermediate 2) (3.0 g; 12.2 mmol; 1.0 eq.), 1-methyl-6-(4,4,5,5,-tetramethyl-1,3,2-dioxaborolan-2-yl)-1H-indole (2.5 g; 9.8 mmol; 1.0 eq.), DIPEA (3.2 g; 24.4 mmol; 2.0 eq.), 1,4-dioxane (16 mL) and water (16 mL). The suspension is purged with argon and then Pd(dppf)Cl₂ (0.89 g; 1.22 mmol; 0.10 eq.) is added. RM is sealed and heated at 85° C. for 3 h. After this time, the mixture is filtered through a Celite® pad and the filtrate is diluted with DCM and extracted with water. The organic phase is washed with brine, dried over Na₂SO₄ and then the solvent is evaporated. Crude product is purified by FCC (hexane/EtOAc; gradient) to afford 7-chloro-5-(-1-methyl-1H-indol-6-yl)-quinoxaline (Intermediate 4) (2.2 g; yield 56%; 92% by UPLC) as a yellow solid.

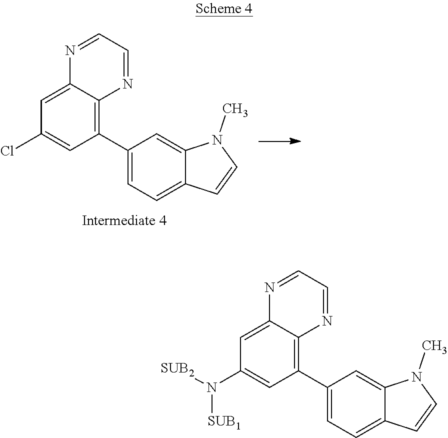

### Example 1 General Procedure 2

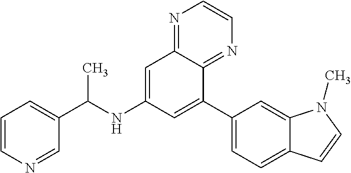 [SMILES: CC(Nc1cc(-c2ccc3ccn(C)c3c2)c2nccnc2c1)c1cccnc1]

A sealed tube is charged with 7-chloro-5-(1-methyl-1H-indol-6-yl)-quinoxaline (60.00 mg; 0.20 mmol; 1.0 eq.) (Intermediate 4), 1-pyridin-3-yl-ethylamine (0.05 mL; 0.41 mmol; 2.0 eq.), NaOtBu (58.77 mg; 0.61 mmol; 3.00 eq.) and toluene (2.0 mL). RM is purged with argon and then BINAP (25.39 mg; 0.04 mmol; 0.20 eq.) and Pd₂(dba)₃ (18.67 mg; 0.02 mmol; 0.10 eq.) are added. RM is sealed and heated at 110° C. for 16 h. After this time, the mixture is filtered through a Celite® pad and the filtrate is diluted with EtOAc and extracted with water. Combined organic phases are washed with brine, dried over Na₂SO₄. Solvent is evaporated and the residue is purified by FCC (hexane/EtOAc; gradient). [8-(1-Methyl-1H-indol-6-yl)-quinoxalin-6-yl]-(1-pyridin-3-yl-ethyl)-amine is obtained as a yellow powder (60.00 mg; yield 79%; 97% by HPLC).

### Example 2

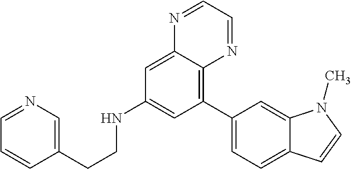 [SMILES: Cn1ccc2ccc(-c3cc(NCCc4cccnc4)cc4nccnc34)cc21]

The product is prepared according to General Procedure 2, described in Example 1 with 7-chloro-5-(1-methyl-1H-indol-6-yl)-quinoxaline (Intermediate 4) (60.00 mg; 0.20 mmol; 1.00 eq.), 2-pyridin-3-yl-ethylamine (0.05 mL; 0.41 mmol; 2.00 eq.), NaOtBu (58.77 mg; 0.61 mmol; 3.00 eq.), BINAP (25.39 mg; 0.04 mmol; 0.20 eq.), Pd₂(dba)₃ (18.67 mg; 0.02 mmol; 0.10 eq.) and toluene (1.5 mL). Reaction is carried out in a MW reactor at 150° C. for 30 min. Purification by FCC (hexane/EtOAc; gradient). [8-(1-Methyl-1H-indol-6-yl)-quinoxalin-6-yl]-(2-pyridin-3-yl-ethyl)-amine (45.00 mg; yield 57%; 97% by HPLC) is obtained as a yellow powder.

### Example 3

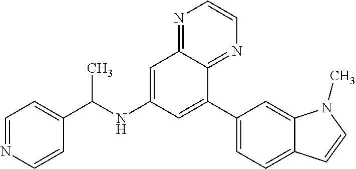 [SMILES: CC(Nc1cc(-c2ccc3ccn(C)c3c2)c2nccnc2c1)c1ccncc1]

The product is prepared according to General Procedure 2, described in Example 1 with 7-chloro-5-(1-methyl-1H-indol-6-yl)-quinoxaline (Intermediate 4) (60.00 mg; 0.20 mmol; 1.00 eq.), 1-pyridin-4-yl-ethylamine (0.05 g; 0.40 mmol; 2.00 eq.), NaOtBu (58.77 mg; 0.61 mmol; 3.00 eq.), BINAP (25.39 mg; 0.04 mmol; 0.20 eq.), Pd₂(dba)₃ (18.67 mg; 0.02 mmol; 0.10 eq.) and toluene (2.00 mL). Purification by FCC (DCM/MeOH; gradient). [8-(1-Methyl-1H-indol-6-yl)-quinoxalin-6-yl]-(1-pyridin-4-yl-ethyl)-amine (55.00 mg; yield 69%; 97% by HPLC) is obtained as a yellow powder.

### Example 4

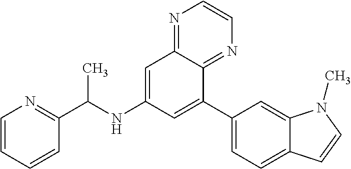 [SMILES: CC(Nc1cc(-c2ccc3ccn(C)c3c2)c2nccnc2c1)c1ccccn1]

The product is prepared according to General Procedure 2, described in Example 1 with 7-chloro-5-(1-methyl-1H-indol-6-yl)-quinoxaline (Intermediate 4) (60.00 mg; 0.20 mmol; 1.00 eq.), 1-pyridin-2-yl-ethylamine (0.05 mL; 0.40 mmol; 2.00 eq.), NaOtBu (58.77 mg; 0.60 mmol; 3.00 eq.), BINAP (25.39 mg; 0.04 mmol; 0.20 eq.), Pd₂(dba)₃ (18.67 mg; 0.02 mmol; 0.10 eq.) and toluene (2.00 mL). Purification by FCC (DCM/MeOH; gradient). Repurification by preparative HPLC and after evaporation extraction with saturated NaHCO₃ solution is done. [8-(1-Methyl-1H-indol-6-yl)-quinoxalin-6-yl]-(1-pyridin-2-yl-ethyl)-amine (0.06 g; yield 78%; 99% by HPLC) is obtained as a yellow powder.

### Example 5

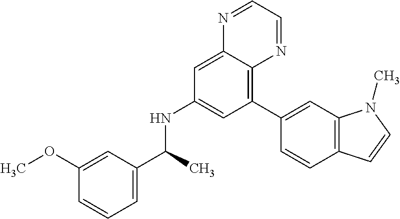 [SMILES: COc1cccc([C@H](C)Nc2cc(-c3ccc4ccn(C)c4c3)c3nccnc3c2)c1]

The product is prepared according to General Procedure 2, described in Example 1 with 7-chloro-5-(1-methyl-1H-indol-6-yl)-quinoxaline (Intermediate 4) (136.00 mg; 0.44 mmol; 1.00 eq.), (S)-1-(3-methoxyphenyl)-ethylamine (157.93 mg; 1.04 mmol; 2.40 eq.), NaOtBu (125.47 mg; 1.31 mmol; 3.00 eq.), BINAP (54.20 mg; 0.09 mmol; 0.20 eq.), Pd₂(dba)₃ (42.31 mg; 0.04 mmol; 0.10 eq.) and toluene (4.00 mL). Purification by FCC (hexane/EtOAc; gradient). [(S)-1-(3-methoxy-phenyl)-ethyl]-[8-(1-methyl-1H-indol-6-yl)-quinoxalin-6-yl]-amine (87.60 mg; yield 48%; 98% by HPLC) is obtained as a brown solid.

### Example 6

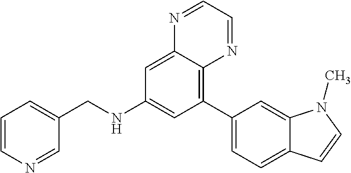 [SMILES: Cn1ccc2ccc(-c3cc(NCc4cccnc4)cc4nccnc34)cc21]

The product is prepared according to General Procedure 2, described in Example 1 with 7-chloro-5-(1-methyl-1H-indol-6-yl)-quinoxaline (Intermediate 4) (60.00 mg; 0.19 mmol; 1.00 eq.), pyridin-3-ylmethylamine (0.04 mL; 0.38 mmol; 2.00 eq.), NaOtBu (54.18 mg; 0.56 mmol; 3.00 eq.), BINAP (23.40 mg; 0.04 mmol; 0.20 eq.), Pd₂(dba)₃ (18.67 mg; 0.02 mmol; 0.10 eq.) and toluene (2.50 mL). Purification by FCC (DCM/MeOH; gradient). [8-(1-Methyl-1H-indol-6-yl)-quinoxalin-6-yl]-pyridin-3-ylmethyl-amine (61.00 mg; yield 78%; 90% by HPLC) is obtained as a yellow amorphous solid.

### Example 7

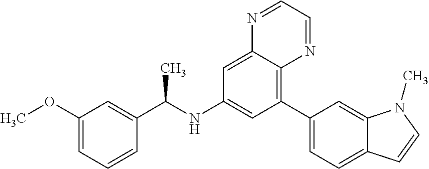 [SMILES: COc1cccc([C@@H](C)Nc2cc(-c3ccc4ccn(C)c4c3)c3nccnc3c2)c1]

The product is prepared according to General Procedure 2, described in Example 1 with 7-chloro-5-(1-methyl-1H-indol-6-yl)-quinoxaline (Intermediate 4) (60.00 mg; 0.19 mmol; 1.00 eq.), (R)-1-(3-methoxy-phenyl)-ethylamine (69.68 mg; 0.46 mmol; 2.40 eq.), NaOtBu (55.36 mg; 0.58 mmol; 3.00 eq.), BINAP (23.91 mg; 0.04 mmol; 0.20 eq.), Pd₂(dba)₃ (17.58 mg; 0.02 mmol; 0.10 eq.) and toluene (4.00 mL). Purification by FCC (hexane/EtOAc; gradient). [(R)-1-(3-Methoxyphenyl)-ethyl]-[8-(1-methyl-1H-indol-6-yl)-quinoxalin-6-yl]-amine (30.00 mg; yield 37%; 96% by HPLC) is obtained as a yellow amorphous powder.

### Example 8

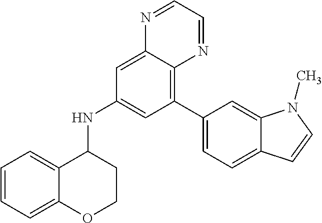 [SMILES: Cn1ccc2ccc(-c3cc(NC4CCOc5ccccc54)cc4nccnc34)cc21]

The product is prepared according to General Procedure 2, described in Example 1 with 7-chloro-5-(1-methyl-1H-indol-6-yl)-quinoxaline (Intermediate 4) (60.00 mg; 0.19 mmol; 1.00 eq.), 3,4-dihydro-2H-chromen-4-ylamine (0.03 mL; 0.23 mmol; 1.20 eq.), NaOtBu (22.38 mg; 0.23 mmol; 1.20 eq.), BINAP (2.42 mg; 0.0039 mmol; 0.20 eq.), Pd₂(dba)₃ (0.018 mg; 0.0019 mmol; 0.10 eq.) and toluene (2.00 mL). Purification by FCC (hexane/EtOAc; gradient). N-(3,4-Dihydro-2H-1-benzopyran-4-yl)-8-(1-methyl-1H-indol-6-yl)quinoxalin-6-amine (20.00 mg; yield 25%; 98% by HPLC) is obtained as a yellow powder.

### Example 9

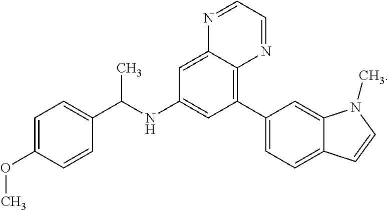 [SMILES: COc1ccc(C(C)Nc2cc(-c3ccc4ccn(C)c4c3)c3nccnc3c2)cc1]

The product is prepared according to General Procedure 2, described in Example 1 with 7-chloro-5-(1-methyl-1H-indol-6-yl)-quinoxaline (Intermediate 4) (60.00 mg; 0.19 mmol; 1.00 eq.), 1-(4-methoxyphenyl)-ethylamine (69.68 mg; 0.46 mmol; 2.40 eq.), NaOtBu (55.36 mg; 0.58 mmol; 3.00 eq.), BINAP (23.91 mg; 0.04 mmol; 0.20 eq.), Pd₂(dba)₃ (17.58 mg; 0.02 mmol; 0.10 eq.) and toluene (4.00 mL). Purification by FCC (hexane/EtOAc; gradient). N-[1-(4-methoxyphenyl)ethyl]-8-(1-methyl-1H-indol-6-yl)quinoxalin-6-amine (22.50 mg; yield 28%; 98% by HPLC) is obtained as a yellow amorphous powder.

### Example 10

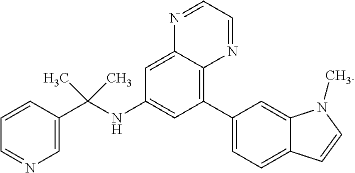 [SMILES: Cn1ccc2ccc(-c3cc(NC(C)(C)c4cccnc4)cc4nccnc34)cc21]

The product is prepared according to General Procedure 2, described in Example 1 with 7-chloro-5-(1-methyl-1H-indol-6-yl)-quinoxaline (Intermediate 4) (70.00 mg; 0.21 mmol; 1.00 eq.), 1-methyl-1-pyridin-3-yl-ethylamine (2-(pyridin-3-yl)propan-2-amine) (70.10 mg; 0.51 mmol; 2.40 eq.), NaOtBu (61.83 mg; 0.64 mmol; 3.00 eq.), BINAP (26.71 mg; 0.04 mmol; 0.20 eq.), Pd₂(dba)₃ (19.64 mg; 0.02 mmol; 0.10 eq.) and toluene (2.00 mL). Purification by FCC (DCM/MeOH; gradient). 8-(1-methyl-1H-indol-6-yl)-N-[2-(pyridin-3-yl)propan-2-yl]quinoxalin-6-amine (34.90 mg; yield 41%; 100% by HPLC) is obtained as a yellow amorphous powder.

### Example 11

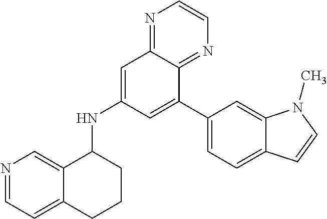 [SMILES: Cn1ccc2ccc(-c3cc(NC4CCCc5ccncc54)cc4nccnc34)cc21]

The product is prepared according to General Procedure 2, described in Example 1 with 7-chloro-5-(1-methyl-1H-indol-6-yl)-quinoxaline (Intermediate 4) (60.00 mg; 0.20 mmol; 1.00 eq.), 5,6,7,8-tetrahydroisoquinolin-8-ylamine dihydrochloride (89.43 mg; 0.40 mmol; 2.00 eq.), NaOtBu (58.30 mg; 0.61 mmol; 3.00 eq.), BINAP (25.18 mg; 0.04 mmol; 0.20 eq.), Pd₂(dba)₃ (18.52 mg; 0.02 mmol; 0.10 eq.) and toluene (1.00 mL). Purification by FCC (hexane/EtOAc; gradient). Repurification by preparative HPLC and after evaporation extraction with saturated NaHCO₃ solution is done. 8-(1-methyl-1H-indol-6-yl)-N-(5,6,7,8-tetrahydroisoquinolin-8-yl)quinoxalin-6-amine (45.00 mg; yield 55%; 99% by HPLC) is obtained as a yellow powder.

### Example 12

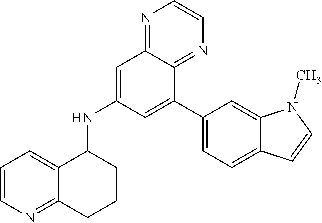 [SMILES: Cn1ccc2ccc(-c3cc(NC4CCCc5ncccc54)cc4nccnc34)cc21]

The product is prepared according to General Procedure 2, described in Example 1 with 7-chloro-5-(1-methyl-1H-indol-6-yl)-quinoxaline (Intermediate 4) (60.00 mg; 0.20 mmol; 1.00 eq.), 5,6,7,8-tetrahydroquinolin-5-ylamine hydrochloride (89.43 mg; 0.40 mmol; 2.00 eq.), NaOtBu (58.30 mg; 0.61 mmol; 3.00 eq.), BINAP (25.18 mg; 0.04 mmol; 0.20 eq.), Pd₂(dba)₃ (18.52 mg; 0.02 mmol; 0.10 eq.) and toluene (1.00 mL). Purification by FCC (hexane/EtOAc; gradient, then EtOAc/MeOH; gradient). 8-(1-Methyl-1H-indol-6-yl)-N-(5,6,7,8-tetrahydroquinolin-5-yl)quinoxalin-6-amine (60.00 mg; yield 69%; 94% by HPLC) is obtained as a yellow powder.

### Example 13

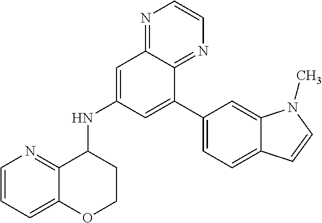 [SMILES: Cn1ccc2ccc(-c3cc(NC4CCOc5cccnc54)cc4nccnc34)cc21]

The product is prepared according to General Procedure 2, described in Example 1 with 7-chloro-5-(1-methyl-1H-indol-6-yl)-quinoxaline (Intermediate 4) (50.00 mg; 0.17 mmol; 1.00 eq.), 3,4-dihydro-2H-pyrano[3,2-b]pyridin-4-ylamine (59.57 mg; 0.40 mmol; 2.40 eq.), NaOtBu (47.65 mg; 0.50 mmol; 3.00 eq.), BINAP (20.58 mg; 0.03 mmol; 0.20 eq.), Pd₂(dba)₃ (15.13 mg; 0.02 mmol; 0.10 eq.) and toluene (4.00 mL). Purification by FCC (hexane/EtOAc; gradient). (3,4-Dihydro-2H-pyrano[3,2-b]pyridin-4-yl)-[8-(1-methyl-1H-indol-6-yl)-quinoxalin-6-yl]-amine (60.00 mg; yield 89%; 99% by HPLC) is obtained as a yellow solid.

### Example 14

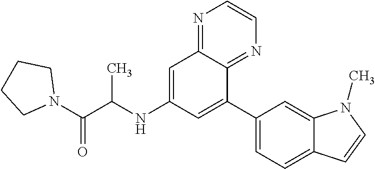 [SMILES: CC(Nc1cc(-c2ccc3ccn(C)c3c2)c2nccnc2c1)C(=O)N1CCCC1]

The product is prepared according to General Procedure 2, described in Example 1 with 7-chloro-5-(1-methyl-1H-indol-6-yl)-quinoxaline (Intermediate 4) (50.00 mg; 0.16 mmol; 1.00 eq.), 2-amino-1-pyrrolidin-1-yl-propan-1-one (55.19 mg; 0.39 mmol; 2.40 eq.), NaOtBu (46.62 mg; 0.49 mmol; 3.00 eq.), BINAP (20.14 mg; 0.03 mmol; 0.20 eq.), Pd₂(dba)₃ (37.54 mg; 0.02 mmol; 0.10 eq.) and toluene (4.00 mL). Purification by FCC (DCM/MeOH; gradient). 2-{[8-(1-Methyl-1H-indol-6-yl)quinoxalin-6-yl]amino}-1-(pyrrolidin-1-yl)propan-1-one (47.00 mg; yield 70%; 97% by HPLC) is obtained as a yellow amorphous powder.

### Example 15

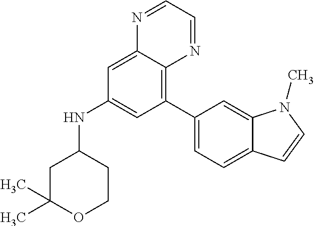 [SMILES: Cn1ccc2ccc(-c3cc(NC4CCOC(C)(C)C4)cc4nccnc34)cc21]

The product is prepared according to General Procedure 2, described in Example 1 with 7-chloro-5-(1-methyl-1H-indol-6-yl)-quinoxaline (Intermediate 4) (85.00 mg; 0.29 mmol; 1.00 eq.), 2,2-dimethyltetrahydropyran-4-ylamine (74.77 mg; 0.58 mmol; 2.00 eq.), NaOtBu (83.43 mg; 0.87 mmol; 3.00 eq.), BINAP (36.04 mg; 0.06 mmol; 0.20 eq.), Pd₂(dba)₃ (26.50 mg; 0.03 mmol; 0.10 eq.) and toluene (3.00 mL). Purification by FCC (hexane/EtOAc; gradient). Repurification by preparative HPLC and after evaporation extraction with saturated NaHCO₃ solution is done. N-(2,2-dimethyloxan-4-yl)-8-(1-methyl-1H-indol-6-yl)quinoxalin-6-amine (77.00 mg; yield 69%; 100% by HPLC) is obtained as a yellow brown powder.

### Example 16

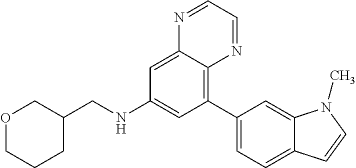 [SMILES: Cn1ccc2ccc(-c3cc(NCC4CCCOC4)cc4nccnc34)cc21]

The product is prepared according to General Procedure 2, described in Example 1 with 7-chloro-5-(1-methyl-1H-indol-6-yl)-quinoxaline (Intermediate 4) (85.00 mg; 0.29 mmol; 1.00 eq.), (tetrahydro-2H-pyran-3-yl)methylamine (66.65 mg; 0.58 mmol; 2.00 eq.), NaOtBu (83.43 mg; 0.87 mmol; 3.00 eq.), BINAP (36.04 mg; 0.06 mmol; 0.20 eq.), Pd₂(dba)₃ (26.50 mg; 0.03 mmol; 0.10 eq.) and toluene (3.00 mL). Purification by FCC (hexane/EtOAc; gradient). Repurification by preparative HPLC and after evaporation extraction with saturated NaHCO₃ solution is done. [8-(1-Methyl-1H-indol-6-yl)-quinoxalin-6-yl]-(tetrahydropyran-3-ylmethyl)amine (87.00 mg; yield 80%; 100% by HPLC) is obtained as a yellow-brown powder.

### Example 17

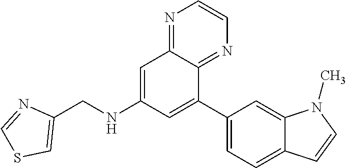 [SMILES: Cn1ccc2ccc(-c3cc(NCc4cscn4)cc4nccnc34)cc21]

The product is prepared according to General Procedure 2, described in Example 1 with 7-chloro-5-(1-methyl-1H-indol-6-yl)-quinoxaline (Intermediate 4) (50.00 mg; 0.16 mmol; 1.00 eq.), 1,3-thiazol-4-ylmethylamine hydrochloride (48.71 mg; 0.32 mmol; 2.00 eq.), NaOtBu (54.39 mg; 0.57 mmol; 3.50 eq.), BINAP (20.14 mg; 0.03 mmol; 0.20 eq.), Pd₂(dba)₃ (14.81 mg; 0.02 mmol; 0.10 eq.) and toluene (2.50 mL). Reaction is carried out in a MW reactor at 160° C. for 1 h. Purification by FCC (hexane/EtOAc; gradient). Repurification by preparative HPLC. [8-(1-Methyl-1H-indol-6-yl)-quinoxalin-6-yl]-thiazol-4-ylmethyl-amine trifluoroacetate (14.00 mg; yield 16%; 91% by HPLC) is obtained as a red amorphous solid.

### Example 18—General Procedure 3

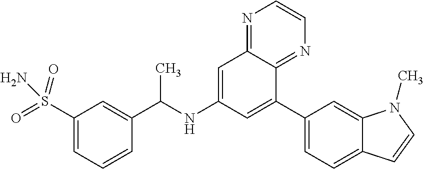 [SMILES: CC(Nc1cc(-c2ccc3ccn(C)c3c2)c2nccnc2c1)c1cccc(S(N)(=O)=O)c1]

A sealed tube is charged with 7-chloro-5-(1-methyl-1H-indol-6-yl)-quinoxaline (Intermediate 4) (100.00 mg; 0.34 mmol; 1.00 eq.), BrettPhos (12.79 mg; 0.02 mmol; 0.07 eq.) and BrettPhos precatalyst (19.04 mg; 0.02 mmol; 0.07 eq.). RM is sealed and then degassed and purged with argon twice. Then LiHMDS 1.0 M in THF (1.16 mL; 1.16 mmol; 3.40 eq.) and 3-(1-aminoethyl)-benzenesulfonamide (115.89 mg; 0.58 mmol; 1.70 eq.) are added by syringe. RM is stirred at 65° C. for 16 h and after this time, RM is diluted with MeOH. Solvents are evaporated and the residue is purified by FCC (hexane/EtOAc; gradient). 3-{1-[8-(1-Methyl-1H-indol-6-yl)-quinoxalin-6-ylamino]-ethyl}-benzenesulfonamide (12.00 mg; yield 8%; 98% by HPLC) is obtained as a yellow powder.

### Example 19

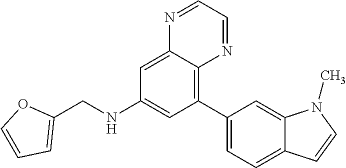 [SMILES: Cn1ccc2ccc(-c3cc(NCc4ccco4)cc4nccnc34)cc21]

The product is prepared according to General Procedure 2, described in Example 1 with 7-chloro-5-(1-methyl-1H-indol-6-yl)-quinoxaline (Intermediate 4) (136.00 mg; 0.44 mmol; 1.00 eq.), C-furan-2-ylmethanamine (101.43 mg; 1.04 mmol; 2.40 eq.), NaOtBu (125.47 mg; 1.31 mmol; 3.00 eq.), BINAP (54.20 mg; 0.09 mmol; 0.20 eq.), Pd₂(dba)₃ (42.31 mg; 0.04 mmol; 0.10 eq.) and toluene (4.00 mL). Reaction is carried out in a MW reactor at 160° C. for 1 h. Purification by FCC (hexane/EtOAc; gradient). Furan-2-ylmethyl-[8-(1-methyl-1H-indol-6-yl)-quinoxalin-6-yl]-amine (57.00 mg; yield 36%; 97% by HPLC) is obtained as a yellow amorphous powder.

### Example 20

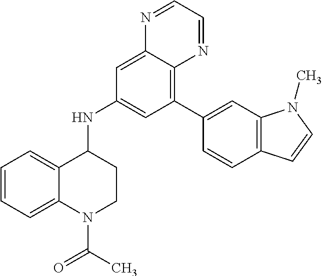 [SMILES: CC(=O)N1CCC(Nc2cc(-c3ccc4ccn(C)c4c3)c3nccnc3c2)c2ccccc21]

The product is prepared according to General Procedure 2, described in Example 1 with 7-chloro-5-(1-methyl-1H-indol-6-yl)-quinoxaline (Intermediate 4) (100.00 mg; 0.34 mmol; 1.00 eq.), 1-(4-amino-3,4-dihydro-2H-quinolin-1-yl)-ethanone (129.53 mg; 0.68 mmol; 2.00 eq.), NaOtBu (98.14 mg; 1.02 mmol; 3.00 eq.), BINAP (42.39 mg; 0.07 mmol; 0.20 eq.), Pd₂(dba)₃ (31.17 mg; 0.03 mmol; 0.10 eq.) and toluene (5.00 mL). Purification by FCC (hexane/EtOAc; gradient). 1-(4-{[8-(1-methyl-1H-indol-6-yl)quinoxalin-6-yl]amino}-1,2,3,4-tetrahydroquinolin-1-yl)ethan-1-one (70.00 mg; yield 46%; 96% by HPLC) is obtained as a yellow powder.

### Example 21

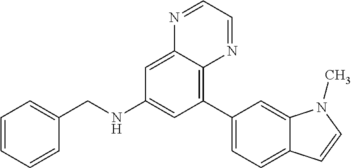 [SMILES: Cn1ccc2ccc(-c3cc(NCc4ccccc4)cc4nccnc34)cc21]

The product is prepared according to General Procedure 2, described in Example 1 with 7-chloro-5-(1-methyl-1H-indol-6-yl)-quinoxaline (Intermediate 4) (100.00 mg; 0.34 mmol; 1.00 eq.), benzylamine (0.06 mL; 0.51 mmol; 1.50 eq.), NaOtBu (98.14 mg; 1.02 mmol; 3.00 eq.), BINAP (9.41 mg; 0.07 mmol; 0.20 eq.), Pd₂(dba)₃ (31.17 mg; 0.03 mmol; 0.10 eq.) and toluene (10.00 mL). Purification by FCC (hexane/EtOAc; gradient). Benzyl-[8-(1-methyl-1H-indol-6-yl)quinoxalin-6-yl]amine (112.00 mg; yield 87%; 96% by HPLC) is obtained as a yellow powder.

### Example 22

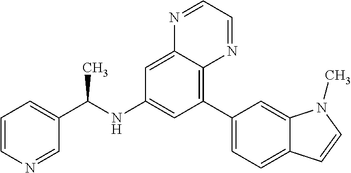 [SMILES: C[C@@H](Nc1cc(-c2ccc3ccn(C)c3c2)c2nccnc2c1)c1cccnc1]

The product is prepared according to General Procedure 2, described in Example 1 with 7-chloro-5-(1-methyl-1H-indol-6-yl)-quinoxaline (Intermediate 4) (100.00 mg; 0.34 mmol; 1.00 eq.), (R)-1-pyridin-3-yl-ethylamine hydrochloride (132.83 mg; 0.68 mmol; 2.00 eq.), NaOtBu (163.58 mg; 1.70 mmol; 5.00 eq.), BINAP (42.39 mg; 0.07 mmol; 0.20 eq.), Pd₂(dba)₃ (31.17 mg; 0.03 mmol; 0.10 eq.) and toluene (3.00 mL). Purification by FCC (DCM/MeOH; gradient). 8-(1-methyl-1H-indol-6-yl)-N-[(1R)-1-(pyridin-3-yl)ethyl]quinoxalin-6-amine (55.00 mg; yield 41%; 97% by HPLC) is obtained as a yellow powder.

### Example 23

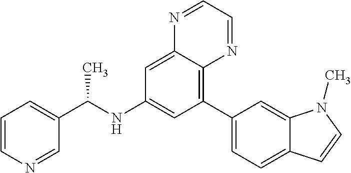 [SMILES: C[C@H](Nc1cc(-c2ccc3ccn(C)c3c2)c2nccnc2c1)c1cccnc1]

The product is prepared according to General Procedure 2, described in Example 1 with 7-chloro-5-(1-methyl-1H-indol-6-yl)-quinoxaline (Intermediate 4) (60.00 mg; 0.20 mmol; 1.00 eq.), (S)-1-pyridin-3-yl-ethylamine (49.91 mg; 0.41 mmol; 2.00 eq.), NaOtBu (98.15 mg; 1.02 mmol; 5.00 eq.), BINAP (25.44 mg; 0.04 mmol; 0.20 eq.), Pd₂(dba)₃ (18.70 mg; 0.02 mmol; 0.10 eq.) and toluene (3.00 mL). Reaction is carried out in a MW reactor at 160° C. for 1 h. Purification by FCC (hexane/EtOAc; gradient). 8-(1-Methyl-1H-indol-6-yl)-N-[(1S)-1-(pyridin-3-yl)ethyl]quinoxalin-6-amine (53.80 mg; yield 66%; 95% by HPLC) is obtained as a yellow powder.

### Example 24

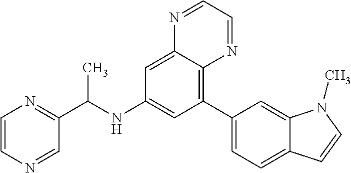 [SMILES: CC(Nc1cc(-c2ccc3ccn(C)c3c2)c2nccnc2c1)c1cnccn1]

The product is prepared according to General Procedure 2, described in Example 1 with 7-chloro-5-(1-methyl-1H-indol-6-yl)-quinoxaline (Intermediate 4) (60.00 mg; 0.20 mmol; 1.00 eq.), 1-pyrazin-2-yl-ethylamine (37.73 mg; 0.31 mmol; 1.50 eq.), NaOtBu (39.26 mg; 0.41 mmol; 2.00 eq.), BINAP (25.44 mg; 0.04 mmol; 0.20 eq.) and Pd₂(dba)₃ (18.70 mg; 0.02 mmol; 0.10 eq.) and toluene (1.00 mL). RM is sealed and heated at 110° C. for 16 h. RM is filtered through a pad of Celite® and product is extracted with DCM. Organic phase is washed with water and brine, dried and concentrated. Purification by FCC (hexane/EtOAc; gradient). 8-(1-Methyl-1H-indol-6-yl)-N-[1-(pyrazin-2-yl)ethyl]quinoxalin-6-amine (51.00 mg; yield 62%; 94% by HPLC) is obtained as a yellow powder.

### Intermediate 5

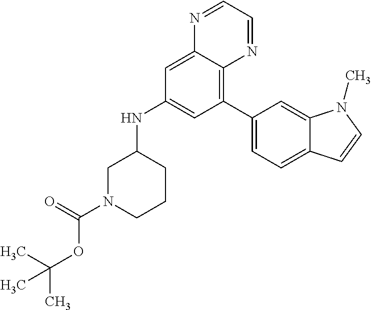 [SMILES: Cn1ccc2ccc(-c3cc(NC4CCCN(C(=O)OC(C)(C)C)C4)cc4nccnc34)cc21]

The product is prepared according to General Procedure 2, described in Example 1 with 7-chloro-5-(1-methyl-1H-indol-6-yl)-quinoxaline (Intermediate 4) (50.00 mg; 0.16 mmol; 1.00 eq.), 3-aminopiperidine-1-carboxylic acid tert-butyl ester (76.91 mg; 0.38 mmol; 2.40 eq.), NaOtBu (46.13 mg; 0.48 mmol; 3.00 eq.), BINAP (19.93 mg; 0.03 mmol; 0.20 eq.), Pd₂(dba)₃ (37.14 mg; 0.02 mmol; 0.10 eq.) and toluene (2.00 mL). Reaction is carried out in a MW reactor at 160° C. for 1 h. Purification by FCC (hexane/EtOAc; gradient). 3-[8-(1-methyl-1H-indol-6-yl)-quinoxalin-6-ylamino]-piperidine-1-carboxylic acid tert-butyl ester (50.90 mg; yield 67%; 97% by UPLC) is obtained as a yellow amorphous powder.

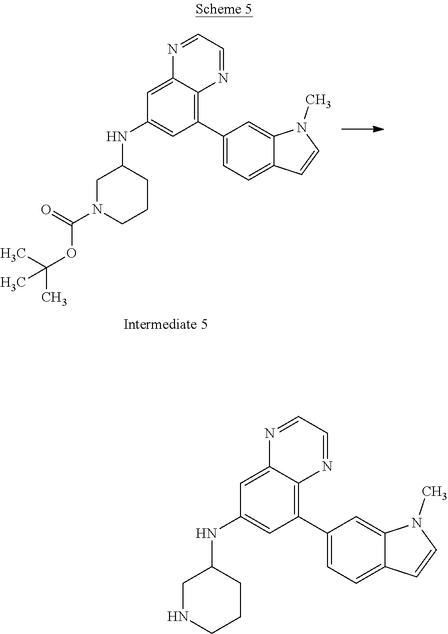 [SMILES: Cn1ccc2ccc(-c3cc(NC4CCCN(C(=O)OC(C)(C)C)C4)cc4nccnc34)cc21 ; Cn1ccc2ccc(-c3cc(NC4CCCNC4)cc4nccnc34)cc21]

### Example 25 General Procedure 4

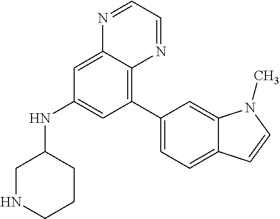 [SMILES: Cn1ccc2ccc(-c3cc(NC4CCCNC4)cc4nccnc34)cc21]

3-[8-(1-Methyl-1H-indol-6-yl)-quinoxalin-6-ylamino]-piperidine-1-carboxylic acid tert-butyl ester (Intermediate 5) (50.90 mg; 0.11 mmol; 1.00 eq.) is dissolved in DCM (1.00 mL), then 4N HCl in 1,4-dioxane (5.00 mL) is added. Resulting mixture is stirred at rt for 6 h. Product is purified by preparative HPLC and after evaporation extraction with saturated NaHCO₃ solution is done. 8-(1-methyl-1H-indol-6-yl)-N-(piperidin-3-yl)quinoxalin-6-amine (14.00 mg; yield 35%; 95% by HPLC) is obtained as a yellow amorphous powder.

### Example 26

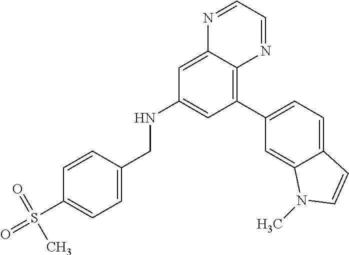 [SMILES: Cn1ccc2ccc(-c3cc(NCc4ccc(S(C)(=O)=O)cc4)cc4nccnc34)cc21]

The product is prepared according to General Procedure 2, described in Example 1 with 7-chloro-5-(1-methyl-1H-indol-6-yl)-quinoxaline (Intermediate 4) (100.00 mg; 0.32 mmol; 1.00 eq.), 4-methanesulfonylbenzylamine (151.35 mg; 0.78 mmol; 2.40 eq.), NaOtBu (93.24 mg; 0.97 mmol; 3.00 eq.), BINAP (41.10 mg; 0.06 mmol; 0.20 eq.), Pd₂(dba)₃ (31.12 mg; 0.03 mmol; 0.10 eq.) and toluene (8.00 mL). Purification by FCC (hexane/EtOAc; gradient). Repurification by preparative HPLC and after evaporation extraction with saturated NaHCO₃ solution is done. N-[(4-Methanesulfonylphenyl)methyl]-8-(1-methyl-1H-indol-6-yl)quinoxalin-6-amine (46.90 mg; yield 31%; 94% by HPLC) is obtained as a red powder.

### Example 27

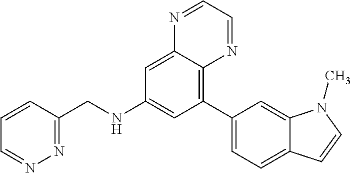 [SMILES: Cn1ccc2ccc(-c3cc(NCc4cccnn4)cc4nccnc34)cc21]

The product is prepared according to General Procedure 2, described in Example 1 with 7-chloro-5-(1-methyl-1H-indol-6-yl)-quinoxaline (Intermediate 4) (60.00 mg; 0.14 mmol; 1.00 eq.), pyridazin-3-ylmethanamine (31.21 mg; 0.29 mmol; 2.00 eq.), NaOtBu (41.22 mg; 0.43 mmol; 3.00 eq.), BINAP (17.81 mg; 0.03 mmol; 0.20 eq.), Pd₂(dba)₃ (13.09 mg; 0.01 mmol; 0.10 eq.) and toluene (5.00 mL). Purification by FCC (DCM/MeOH; gradient). [8-(1-Methyl-1H-indol-6-yl)-quinoxalin-6-yl]-pyridazin-3-ylmethyl-amine (20.00 mg; 37%; 98% by HPLC) is obtained as a dark brown powder.

### Example 28

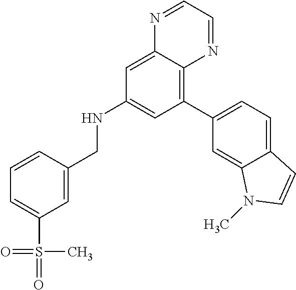 [SMILES: Cn1ccc2ccc(-c3cc(NCc4cccc(S(C)(=O)=O)c4)cc4nccnc34)cc21]

The product is prepared according to General Procedure 2, described in Example 1 with 7-chloro-5-(1-methyl-1H-indol-6-yl)-quinoxaline (Intermediate 4) (100.00 mg; 0.32 mmol; 1.00 eq.), 3-methanesulfonylbenzylamine (151.35 mg; 0.78 mmol; 2.40 eq.), NaOtBu (93.24 mg; 0.97 mmol; 3.00 eq.), BINAP (41.10 mg; 0.06 mmol; 0.20 eq.), Pd₂(dba)₃ (31.12 mg; 0.03 mmol; 0.10 eq.) and toluene (8.00 mL). Reaction is carried out in a MW reactor at 160° C. for 1 h. Purification by FCC (hexane/EtOAc; gradient). (3-Methanesulfonyl-benzyl)-[8-(1-methyl-1H-indol-6-yl)-quinoxalin-6-yl]-amine (59.10 mg; yield 40%; 97% by HPLC) is obtained as a dark yellow powder.

### Example 29

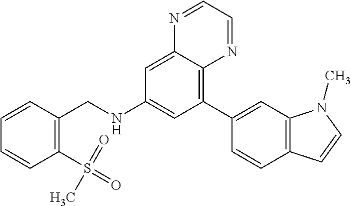 [SMILES: Cn1ccc2ccc(-c3cc(NCc4ccccc4S(C)(=O)=O)cc4nccnc34)cc21]

The product is prepared according to General Procedure 2, described in Example 1 with 7-chloro-5-(1-methyl-1H-indol-6-yl)-quinoxaline (Intermediate 4) (100.00 mg; 0.32 mmol; 1.00 eq.), 2-methanesulfonylbenzylamine (143.78 mg; 0.78 mmol; 2.40 eq.), NaOtBu (93.24 mg; 0.97 mmol; 3.00 eq.), BINAP (41.10 mg; 0.06 mmol; 0.20 eq.), Pd₂(dba)₃ (31.12 mg; 0.03 mmol; 0.10 eq.) and toluene (8.00 mL). Reaction is carried out in a MW reactor at 160° C. for 2 h. Purification by FCC (hexane/EtOAc; gradient). Repurification by preparative HPLC and after evaporation extraction with saturated NaHCO₃ solution is done. N-[(2-Methanesulfonylphenyl)methyl]-8-(1-methyl-1H-indol-6-yl)quinoxalin-6-amine (21.00 mg; 14%; 98% by HPLC) is obtained as a red powder.

### Example 30 General Procedure 5

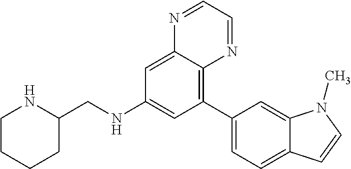 [SMILES: Cn1ccc2ccc(-c3cc(NCC4CCCCN4)cc4nccnc34)cc21]

A sealed tube is charged with 7-chloro-5-(1-methyl-1H-indol-6-yl)-quinoxaline (Intermediate 4) (100.00 mg; 0.34 mmol; 1.00 eq.), 2-piperidylmethylamine (93.29 mg; 0.82 mmol; 2.40 eq.), NaOtBu (45.80 mg; 0.48 mmol; 1.40 eq.) and anhydrous toluene (5.00 mL). RM is purged with argon and then [(Cinnamyl)PdCl]₂ (8.82 mg; 0.02 mmol; 0.05 eq.) and BippyPhos (13.80 mg; 0.03 mmol; 0.08 eq.) are added. RM is sealed and stirred at 110° C. for 12 h. After this time, RM is diluted with DCM, filtered through the Celite® pad. The filtrate is washed with water, brine, dried over Na₂SO₄, filtered and evaporated. Crude product is purified by FCC (DCM/MeOH; gradient). 8-(1-Methyl-1H-indol-6-yl)-N-(piperidin-2-ylmethyl)quinoxalin-6-amine (106.00 mg; yield 80%; 95% by HPLC) is obtained as a yellow solid.

### Example 31

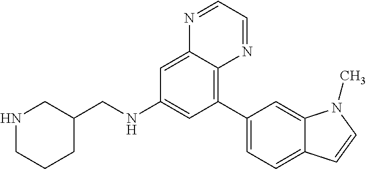 [SMILES: Cn1ccc2ccc(-c3cc(NCC4CCCNC4)cc4nccnc34)cc21]

The product is prepared according to General Procedure 5, described in Example 30 with 7-chloro-5-(1-methyl-1H-indol-6-yl)-quinoxaline (Intermediate 4) (100.00 mg; 0.34 mmol; 1.00 eq.), 3-piperidylmethylamine (93.29 mg; 0.82 mmol; 2.40 eq.), NaOtBu (45.80 mg; 0.48 mmol; 1.40 eq.), [(Cinnamyl)PdCl]₂ (8.82 mg; 0.02 mmol; 0.05 eq.), BippyPhos (13.80 mg; 0.03 mmol; 0.08 eq.) and anhydrous toluene (5.00 mL). Purification by FCC (DCM/MeOH; gradient). 8-(1-methyl-1H-indol-6-yl)-N-(piperidin-3-ylmethyl)quinoxalin-6-amine (50.00 mg; yield 38%; 95% by HPLC) is obtained as a yellow solid.

### Example 32

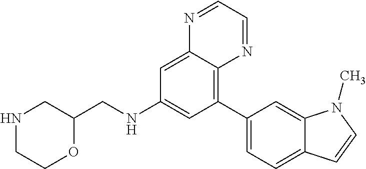 [SMILES: Cn1ccc2ccc(-c3cc(NCC4CNCCO4)cc4nccnc34)cc21]

The product is prepared according to General Procedure 5, described in Example 30 with 7-chloro-5-(1-methyl-1H-indol-6-yl)-quinoxaline (Intermediate 4) (45.00 mg; 0.15 mmol; 1.00 eq.), and morpholin-2-ylmethanamine (21.79 μl; 0.18 mmol; 1.20 eq.), NaOtBu (20.61 mg; 0.21 mmol; 1.40 eq.), [(Cinnamyl)PdCl]₂ (3.97 mg; 0.01 mmol; 0.05 eq.), BippyPhos (7.76 mg; 0.02 mmol; 0.10 eq.), and anhydrous toluene (1.50 mL). Purification by FCC (DCM/MeOH; gradient). Repurification by preparative HPLC and after evaporation extraction with saturated NaHCO₃ solution is done. 8-(1-methyl-1H-indol-6-yl)-N-(morpholin-2-ylmethyl)quinoxalin-6-amine (20.00 mg; yield 35%; 99% by HPLC) is obtained as a yellow powder.

### Example 33

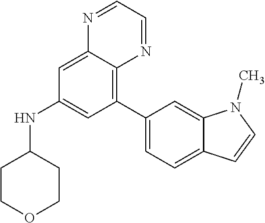 [SMILES: Cn1ccc2ccc(-c3cc(NC4CCOCC4)cc4nccnc34)cc21]

The product is prepared according to General Procedure 5, described in Example 30 with 7-chloro-5-(1-methyl-1H-indol-6-yl)-quinoxaline (Intermediate 4) (100.00 mg; 0.34 mmol; 1.00 eq.), 4-aminotetrahydropyran (41.32 mg; 0.41 mmol; 1.20 eq.), NaOtBu (45.75 mg; 0.48 mmol; 1.40 eq.), bis[(Cinnamyl)PdCl]₂ (8.82 mg; 0.02 mmol; 0.05 eq.), BippyPhos (13.80 mg; 0.03 mmol; 0.08 eq.), anhydrous toluene (5.00 mL). Purification by FCC (DCM/MeOH; gradient). 8-(1-methyl-1H-indol-6-yl)-N-(oxan-4-yl)quinoxalin-6-amine (74.00 mg; yield 58%; 96% by HPLC) is obtained as a yellow powder.

### Example 34

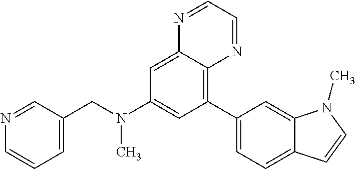 [SMILES: CN(Cc1cccnc1)c1cc(-c2ccc3ccn(C)c3c2)c2nccnc2c1]

The product is prepared according to General Procedure 2, described in Example 1 with 7-chloro-5-(1-methyl-1H-indol-6-yl)-quinoxaline (Intermediate 4) (75.00 mg; 0.26 mmol; 1.00 eq.), N-methyl-1-(pyridin-3-yl)methanamine (35.87 mg; 0.29 mmol; 1.15 eq.), NaOtBu (73.53 mg; 0.77 mmol; 3.00 eq.), BINAP (31.80 mg; 0.05 mmol; 0.20 eq.), Pd₂(dba)₃ (23.38 mg; 0.03 mmol; 0.10 eq.) and anhydrous toluene (5.00 mL). Purification by FCC (EtOAc/MeOH; gradient). N-Methyl-8-(1-methyl-1H-indol-6-yl)-N-(pyridin-3-ylmethyl)quinoxalin-6-amine (37.00 mg; yield 36%; 95% by HPLC) is obtained as a yellow powder.

### Intermediate 6—General Procedure 6

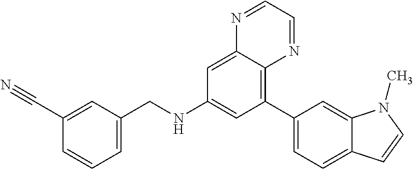 [SMILES: Cn1ccc2ccc(-c3cc(NCc4cccc(C#N)c4)cc4nccnc34)cc21]

A sealed tube is charged with 7-chloro-5-(1-methyl-1H-indol-6-yl)-quinoxaline (175.00 mg; 0.57 mmol; 1.00 eq.) (Intermediate 4), 3-aminomethylbenzonitrile (0.11 mL; 0.85 mmol; 1.50 eq.), Cs₂CO₃ (558.79 mg; 1.70 mmol; 3.00 eq.) and 1,4-dioxane (10.00 mL). RM is purged with argon and then BINAP (17.98 mg; 0.03 mmol; 0.05 eq.) and Pd(OAc)₂ (6.69 mg; 0.03 mmol; 0.05 eq.) are added. RM is sealed and stirred at 150° C. for 1 h. After this time, the mixture is filtered through a Celite® pad and the filtrate is diluted with EtOAc and extracted with water. Combined organic phases are washed with brine, dried over Na₂SO₄. Solvent is evaporated and the residue is purified by FCC (hexane/EtOAc; gradient). 3-({[8-(1-Methyl-1H-indol-6-yl)quinoxalin-6-yl]amino}methyl)benzonitrile (193.00 mg; yield 86%; 98% by HPLC) is obtained as a yellow powder.

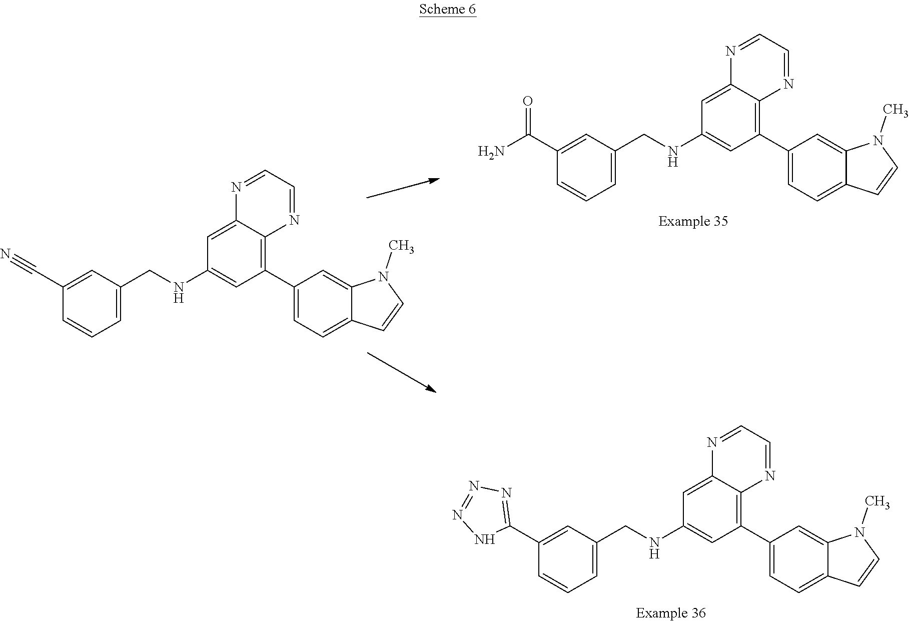 [SMILES: Cn1ccc2ccc(-c3cc(NCc4cccc(C#N)c4)cc4nccnc34)cc21 ; Cn1ccc2ccc(-c3cc(NCc4cccc(C(N)=O)c4)cc4nccnc34)cc21 ; Cn1ccc2ccc(-c3cc(NCc4cccc(-c5nnn[nH]5)c4)cc4nccnc34)cc21]

### Example 35 General Procedure 7

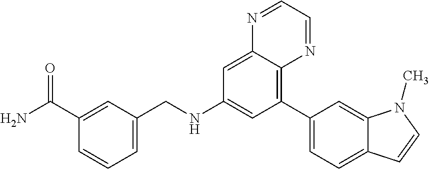 [SMILES: Cn1ccc2ccc(-c3cc(NCc4cccc(C(N)=O)c4)cc4nccnc34)cc21]

A round-bottom flask is charged with solution of KOH (21.61 mg; 0.39 mmol; 3.00 eq.) in t-BuOH (4.00 mL). To this solution 3-{[8-(1-methyl-1H-indol-6-yl)-quinoxalin-6-ylamino]-methyl}-benzonitrile (50.00 mg; 0.13 mmol; 1.00 eq.) (Intermediate 6) is added and then RM is stirred at 80° C. for 3h. After this time, RM is diluted with EtOAc and washed with water and brine. Organic layer is dried with Na₂SO₄, filtered and evaporated. Crude product is purified by FCC (hexane/EtOAc; gradient). 3-({[8-(1-Methyl-1H-indol-6-yl)quinoxalin-6-yl]amino}methyl)benzamide (46.00 mg; yield 85%; 96% by HPLC) is obtained as a yellow powder.

### Example 36 General Procedure 8

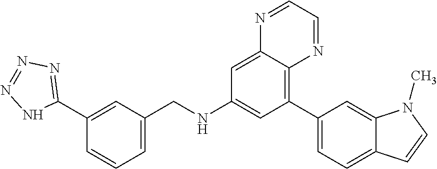 [SMILES: Cn1ccc2ccc(-c3cc(NCc4cccc(-c5nnn[nH]5)c4)cc4nccnc34)cc21]

A sealed tube is charged with 3-{[8-(1-methyl-1H-indol-6-yl)-quinoxalin-6-ylamino]-methyl}-benzonitrile (Intermediate 6) (50.00 mg; 0.13 mmol; 1.00 eq.), NaN₃ (24.86 mg; 0.38 mmol; 3.00 eq.), TEA*HCl (52.64 mg; 0.38 mmol; 3.00 eq.) and anhydrous toluene (5.00 mL). SM is stirred under Ar atmosphere at 110° C. for 20h. After this time, RM is diluted with EtOAc and washed with saturated NaHCO₃ and brine. Organic layer is dried with Na₂SO₄, filtered and evaporated. Crude product is purified by FCC (EtOAc/MeOH; gradient; silica gel, deactivated with NH₃). 8-(1-methyl-1H-indol-6-yl)-N-{[3-(1H-1,2,3,4-tetrazol-5-yl)phenyl]methyl}quinoxalin-6-amine (37.00 mg; yield 63%; 93% by HPLC) is obtained as a red solid.

### Intermediate 7

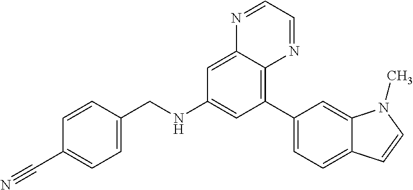 [SMILES: Cn1ccc2ccc(-c3cc(NCc4ccc(C#N)cc4)cc4nccnc34)cc21]

The product is prepared according to General Procedure 2, described in Example 1 with 7-chloro-5-(1-methyl-1H-indol-6-yl)-quinoxaline (Intermediate 4) (125.00 mg; 0.42 mmol; 1.00 eq.), 4-aminomethylbenzonitrile (85.22 mg; 0.63 mmol; 1.50 eq.), NaOtBu (72.36 mg; 0.63 mmol; 1.50 eq.), BINAP (10.71 mg; 0.02 mmol; 0.04 eq.) and Pd₂(dba)₃ (8.12 mg; 0.01 mmol; 0.02 eq.) and 1,4-dioxane (7.50 mL). RM is stirred at 150° C. for 32 h. Purification by FCC (hexane/EtOAc; gradient). 4-({[8-(1-Methyl-1H-indol-6-yl)quinoxalin-6-yl]amino}methyl)benzonitrile (91.00 mg; yield 55%; 99% by HPLC) is obtained as a yellow powder.

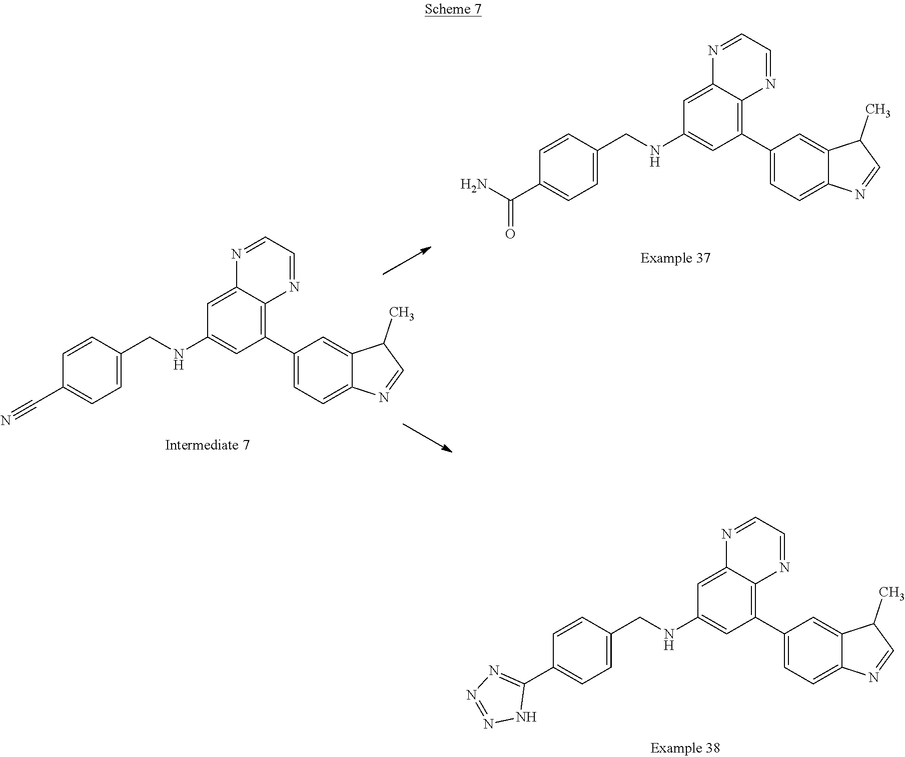 [SMILES: CC1C=Nc2ccc(-c3cc(NCc4ccc(C(N)=O)cc4)cc4nccnc34)cc21 ; CC1C=Nc2ccc(-c3cc(NCc4ccc(C#N)cc4)cc4nccnc34)cc21 ; CC1C=Nc2ccc(-c3cc(NCc4ccc(-c5nnn[nH]5)cc4)cc4nccnc34)cc21]

### Example 37

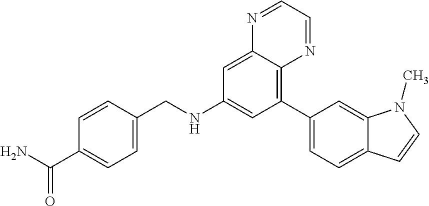 [SMILES: Cn1ccc2ccc(-c3cc(NCc4ccc(C(N)=O)cc4)cc4nccnc34)cc21]

The product is prepared according to General Procedure 7, described in Example 35 with 4-{[8-(1-methyl-1H-indol-6-yl)-quinoxalin-6-ylamino]-methyl}-benzonitrile (Intermediate 7) (41.00 mg; 0.10 mmol; 1.00 eq.), KOH (17.24 mg; 0.31 mmol; 3.00 eq.) and t-BuOH (4.00 mL). Purification by FCC (hexane/EtOAc; gradient then EtOAc/MeOH; gradient). 4-({[8-(1-Methyl-1H-indol-6-yl)quinoxalin-6-yl]amino}methyl)benzamide (29.00 mg; yield 64%; 92% by HPLC) is obtained as a yellow powder.

### Example 38

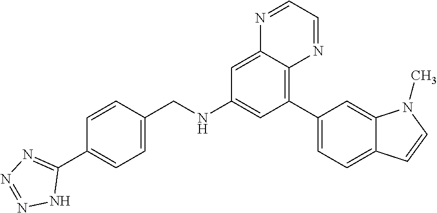 [SMILES: Cn1ccc2ccc(-c3cc(NCc4ccc(-c5nnn[nH]5)cc4)cc4nccnc34)cc21]

The product is prepared according to General Procedure 8, described in Example 36 with 4-{[8-(1-methyl-1H-indol-6-yl)-quinoxalin-6-ylamino]-methyl}-benzonitrile (Intermediate 7) (45.00 mg; 0.11 mmol; 1.00 eq.), NaN₃ (33.35 mg; 0.51 mmol; 4.50 eq.), TEA*HCl (70.61 mg; 0.51 mmol; 4.50 eq.) and anhydrous toluene (5.00 mL). Purification by FCC (EtOAc/MeOH; gradient). 8-(1-Methyl-1H-indol-6-yl)-N-{[4-(1H-1,2,3,4-tetrazol-5-yl)phenyl]methyl}-quinoxalin-6-amine (31.00 mg; yield 59%; 93% by HPLC) is obtained as a yellow powder.

### Example 39

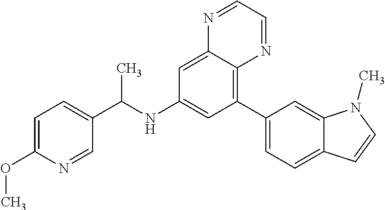 [SMILES: COc1ccc(C(C)Nc2cc(-c3ccc4ccn(C)c4c3)c3nccnc3c2)cn1]

The product is prepared according to General Procedure 2, described in Example 1 with 7-chloro-5-(1-methyl-1H-indol-6-yl)-quinoxaline (Intermediate 4) (100.00 mg; 0.34 mmol; 1.00 eq.), 1-(6-methoxypyridin-3-yl)ethylamine (77.72 mg; 0.51 mmol; 1.50 eq.), NaOtBu (98.14 mg; 1.02 mmol; 3.00 eq.), BINAP (42.39 mg; 0.07 mmol; 0.20 eq.), Pd₂(dba)₃ (31.17 mg; 0.03 mmol; 0.10 eq.) and toluene (5.00 mL). Purification by FCC (hexane/EtOAc; gradient). N-[1-(6-methoxypyridin-3-yl)ethyl]-8-(1-methyl-1H-indol-6-yl)quinoxalin-6-amine (114.00 mg; yield 79%; 96% by HPLC) is obtained as a yellow powder.

### Example 40

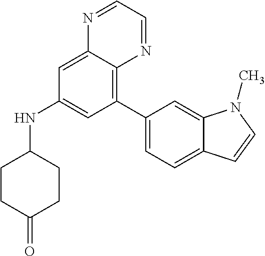 [SMILES: Cn1ccc2ccc(-c3cc(NC4CCC(=O)CC4)cc4nccnc34)cc21]

The product is prepared according to General Procedure 3, described in Example 18 with 7-chloro-5-(1-methyl-1H-indol-6-yl)-quinoxaline (Intermediate 4) (50.00 mg; 0.17 mmol; 1.00 eq.), 4-amino-cyclohexanone hydrochloride (38.20 mg; 0.26 mmol; 1.50 eq.), BrettPhos (5.48 mg; 0.01 mmol; 0.06 eq.), BrettPhos precatalyst (8.16 mg; 0.01 mmol; 0.06 eq.) and LiHMDS 1.0 M in THF (0.51 mL; 0.51 mmol; 3.00 eq.). RM is stirred at 60° C. for 21 h. Purification by FCC (DCM/MeOH; gradient). Repurification by preparative HPLC and after evaporation extraction with saturated NaHCO₃ solution is done. 4-{[8-(1-Methyl-1H-indol-6-yl)quinoxalin-6-yl]amino}cyclohexan-1-one (7.00 mg; 11%; 100% by HPLC) is obtained as a yellow powder.

### Example 41—General Procedure 9

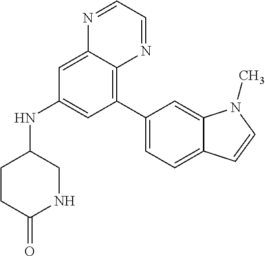 [SMILES: Cn1ccc2ccc(-c3cc(NC4CCC(=O)NC4)cc4nccnc34)cc21]

A sealed tube is charged with 7-chloro-5-(1-methyl-1H-indol-6-yl)-quinoxaline (120.00 mg; 0.40 mmol; 1.00 eq.) (Intermediate 4), 5-aminopiperidin-2-one hydrochloride (73.09 mg; 0.49 mmol; 1.20 eq.), K₂CO₃ (111.78 mg; 0.81 mmol; 2.00 eq.) and 2-methylpropan-2-ol (1.40 mL). RM is purged with argon and then Xphos (38.55 mg; 0.08 mmol; 0.20 eq.) and Pd₂(dba)₃ (18.52 mg; 0.02 mmol; 0.05 eq.) are added. RM is sealed and heated at 100° C. for 24 h. After this time, the mixture is filtered through a Celite® pad and the filtrate is poured into water and obtained mixture is extracted with DCM. The combined organic layers are washed with water, brine, dried over Na₂SO₄ and concentrated in vacuo. The residue is purified by FCC (DCM/MeOH; gradient). 5-{[8-(1-methyl-1H-indol-6-yl)quinoxalin-6-yl]amino}piperidin-2-one (38.00 mg; yield 12.0%; 95% by HPLC) is obtained as a yellow powder.

### Example 42

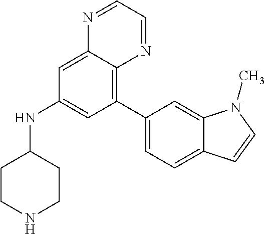 [SMILES: Cn1ccc2ccc(-c3cc(NC4CCNCC4)cc4nccnc34)cc21]

The product is prepared according to General Procedure 5, described in Example 30 with 7-chloro-5-(1-methyl-1H-indol-6-yl)-quinoxaline (Intermediate 4) (50.00 mg; 0.17 mmol; 1.00 eq.), piperidin-4-yl-amine (20.80 μl; 0.20 mmol; 1.20 eq.), NaOtBu (22.21 mg; 0.23 mmol; 1.40 eq.), BippyPhos (8.36 mg; 0.02 mmol; 0.10 eq.), [(Cinnamyl)PdCl]₂ (4.28 mg; 0.01 mmol; 0.05 eq.) and toluene (1.50 mL). Purification by FCC (DCM/MeOH; gradient). 8-(1-Methyl-1H-indol-6-yl)-N-(piperidin-4-yl)quinoxalin-6-amine (28.00 mg; yield 47%; 98% by HPLC) is obtained as a light orange solid.

### Example 43

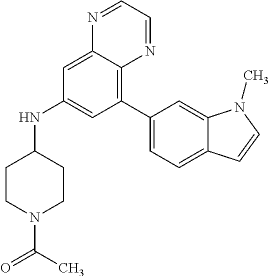 [SMILES: CC(=O)N1CCC(Nc2cc(-c3ccc4ccn(C)c4c3)c3nccnc3c2)CC1]

The product is prepared according to General Procedure 2, described in Example 1 with 7-chloro-5-(1-methyl-1H-indol-6-yl)-quinoxaline (Intermediate 4) (50.00 mg; 0.17 mmol; 1.00 eq.), 1-(4-aminopiperidin-1-yl)-ethanone (48.41 mg; 0.34 mmol; 2.00 eq.), NaOtBu (49.07 mg; 0.51 mmol; 3.00 eq.), BINAP (21.20 mg; 0.03 mmol; 0.20 eq.), Pd₂(dba)₃ (15.59 mg; 0.02 mmol; 0.10 eq.) and toluene (2.00 mL). Purification by FCC (hexane/EtOAc; gradient). 1-(4-{[8-(1-methyl-1H-indol-6-yl)quinoxalin-6-yl]amino}piperidin-1-yl)ethan-1-one (48.60 mg; yield 69%; 96% by HPLC) is obtained as a yellow powder.

### Intermediate 8

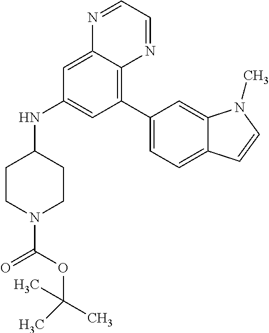 [SMILES: Cn1ccc2ccc(-c3cc(NC4CCN(C(=O)OC(C)(C)C)CC4)cc4nccnc34)cc21]

The product is prepared according to General Procedure 2, described in Example 1 with 7-chloro-5-(1-methyl-1H-indol-6-yl)-quinoxaline (Intermediate 4) (70.00 mg; 0.24 mmol; 1.00 eq.), 4-amino-piperidine-1-carboxylic acid tert-butyl ester (94.50 mg; 0.47 mmol; 2.00 eq.), NaOtBu (68.02 mg; 0.71 mmol; 3.00 eq.), BINAP (29.38 mg; 0.05 mmol; 0.20 eq.), Pd₂(dba)₃ (21.60 mg; 0.02 mmol; 0.10 eq.) and toluene (2.50 mL). Purification by FCC (DCM/MeOH; gradient). 4-[8-(1-Methyl-1H-indol-6-yl)-quinoxalin-6-ylamino]-piperidine-1-carboxylic acid tert-butyl ester (70.00 mg; yield 63%; 97% by UPLC) is obtained as a yellow solid.

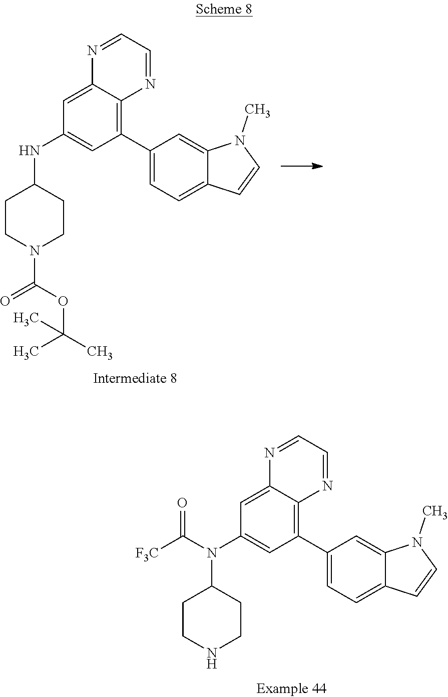 [SMILES: Cn1ccc2ccc(-c3cc(NC4CCN(C(=O)OC(C)(C)C)CC4)cc4nccnc34)cc21 ; Cn1ccc2ccc(-c3cc(N(C(=O)C(F)(F)F)C4CCNCC4)cc4nccnc34)cc21]

### Example 44—General Procedure 10

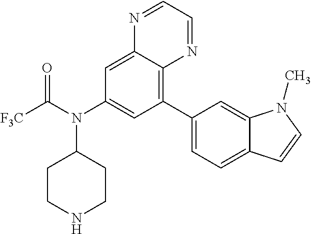 [SMILES: Cn1ccc2ccc(-c3cc(N(C(=O)C(F)(F)F)C4CCNCC4)cc4nccnc34)cc21]

To solution of 4-[8-(1-methyl-1H-indol-6-yl)-quinoxalin-6-ylamino]-piperidine-1-carboxylic acid tert-butyl ester (Intermediate 8) (68.00 mg; 0.15 mmol; 1.00 eq.) in DCM (4.00 mL) mixture of TFAA in DCM is added dropwise. RM is stirred at rt overnight, diluted with DCM and extracted with aqueous solution of NaHCO₃. Organic phase is dried over MgSO₄, filtered and concentrated. 2,2,2-Trifluoro-N-[8-(1-methyl-1H-indol-6-yl)quinoxalin-6-yl]-N-(piperidin-4-yl)acetamide (24.00 mg; yield 32%; 88% by HPLC) is obtained as a yellow solid.

### Example 45

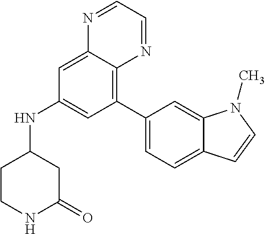 [SMILES: Cn1ccc2ccc(-c3cc(NC4CCNC(=O)C4)cc4nccnc34)cc21]

The product is prepared according to General Procedure 9, described in Example 41 with 7-chloro-5-(1-methyl-1H-indol-6-yl)-quinoxaline (Intermediate 4) (100.00 mg; 0.34 mmol; 1.00 eq.), 4-amino-piperidin-2-one trifluoroacetate (92.28 mg; 0.40 mmol; 1.20 eq.), K₂CO₃ (93.15 mg; 0.67 mmol; 2.00 eq.), Pd₂(dba)₃ (30.86 mg; 0.03 mmol; 0.10 eq.), Xphos (32.13 mg; 0.07 mmol; 0.20 eq.) and 2-methylpropan-2-ol (2.00 mL). Purification by FCC (hexane/EtOAc; gradient). 4-{[8-(1-Methyl-1H-indol-6-yl)quinoxalin-6-yl]amino}piperidin-2-one (110.00 mg; yield 82%; 93% by HPLC) is obtained as a red powder.

### Intermediate 9

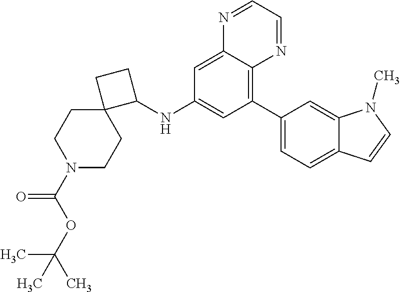 [SMILES: Cn1ccc2ccc(-c3cc(NC4CCC45CCN(C(=O)OC(C)(C)C)CC5)cc4nccnc34)cc21]

The product is prepared according to General Procedure 2, described in Example 1 with 7-chloro-5-(1-methyl-1H-indol-6-yl)-quinoxaline (Intermediate 4) (80.00 mg; 0.26 mmol; 1.00 eq.), 1-amino-7-azaspiro[3.5]nonane-7-carboxylic acid tert-butyl ester (152.54 mg; 0.63 mmol; 2.40 eq.), NaOtBu (76.24 mg; 0.79 mmol; 3.00 eq.), BINAP (32.93 mg; 0.05 mmol; 0.20 eq.), Pd₂(dba)₃ (24.22 mg; 0.03 mmol; 0.10 eq.) and anhydrous toluene (4.00 mL). Purification by FCC (hexane/EtOAc; gradient). 1-[8-(1-Methyl-H-indol-6-yl)-quinoxalin-6-ylamino]-7-aza-spiro[3.5]nonane-7-carboxylic acid tert-butyl ester (110.00 mg; yield 84%; 100% by UPLC) is obtained as an orange solid.

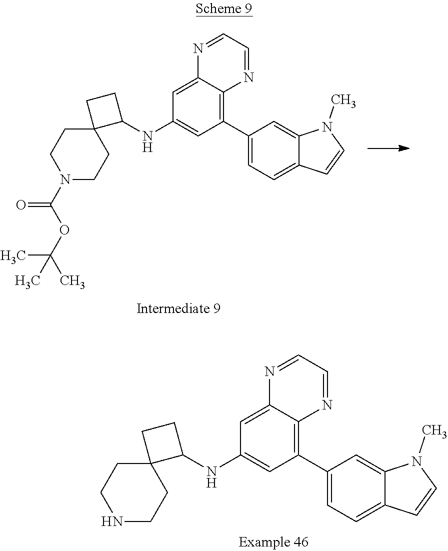 [SMILES: Cn1ccc2ccc(-c3cc(NC4CCC45CCN(C(=O)OC(C)(C)C)CC5)cc4nccnc34)cc21 ; Cn1ccc2ccc(-c3cc(NC4CCC45CCNCC5)cc4nccnc34)cc21]

### Example 46—General Procedure 11

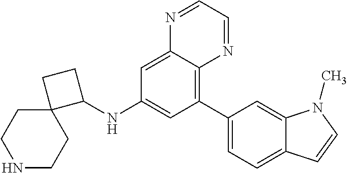 [SMILES: Cn1ccc2ccc(-c3cc(NC4CCC45CCNCC5)cc4nccnc34)cc21]

1-[8-(1-Methyl-1H-indol-6-yl)-quinoxalin-6-ylamino]-7-aza-spiro[3.5]nonane-7-carboxylic acid tert-butyl ester (Intermediate 9) (110.00 mg; 0.22 mmol; 1.00 eq.) is dissolved in DCM (2.00 mL) and to this solution 2M HCl in Et₂O (2.21 mL; 4.42 mmol; 20.00 eq.) is added. RM is stirred at rt overnight and then EtOAc and 1 M NaOH (5 mL) are added. Organic layer is washed with brine, dried over Na₂SO₄, filtered and evaporated. Purification by preparative HPLC. N-{7-Azaspiro[3.5]nonan-1-yl}-8-(1-methyl-1H-indol-6-yl)quinoxalin-6-amine (45.00 mg; yield 45%; 88% by HPLC) is obtained as a yellow solid.

### Example 47

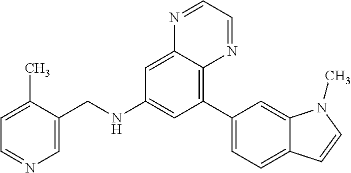 [SMILES: Cc1ccncc1CNc1cc(-c2ccc3ccn(C)c3c2)c2nccnc2c1]

The product is prepared according to General Procedure 6, described for Intermediate 6 with 7-chloro-5-(1-methyl-1H-indol-6-yl)-quinoxaline (Intermediate 4) (50.0 mg, 0.16 mmol; 1.0 eq.), (4-methylpyridin-3-yl)methylamine (31.19 mg; 0.24 mmol; 1.50 eq.), Cs₂CO₃ (159.65 mg; 0.49 mmol; 3.00 eq.), BINAP (10.27 mg; 0.02 mmol; 0.10 eq.), Pd(OAc)₂ (3.82 mg; 0.02 mmol; 0.10 eq.) and 1,4-dioxane (2.00 mL. Purification by FCC (hexane/EtOAc: gradient). [8-(1-Methyl-1H-indol-6-yl)-quinoxalin-6-yl]-(4-methyl-pyridin-3-ylmethyl)-amine (50.00 mg; yield 78%; 95% by HPLC) is obtained as a yellow powder.

### Example 48

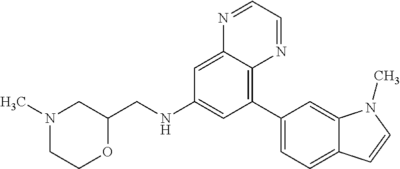 [SMILES: CN1CCOC(CNc2cc(-c3ccc4ccn(C)c4c3)c3nccnc3c2)C1]

The product is prepared according to General Procedure 2, described in Example 1 with 7-chloro-5-(1-methyl-1H-indol-6-yl)-quinoxaline (Intermediate 4) (50.0 mg, 0.11 mmol; 1.0 eq.), C-(4-Methyl-morpholin-2-yl)-methylamine (45.41 μl; 0.34 mmol; 2.00 eq.), NaOtBu (49.07 mg; 0.51 mmol; 3.00 eq.), BINAP (21.63 mg; 0.03 mmol; 0.20 eq.), Pd₂(dba)₃ (15.59 mg; 0.02 mmol; 0.10 eq.) and toluene (5.0 mL). Purification by FCC (hexane/EtOAc; gradient). Repurification by preparative HPLC. [8-(1-Methyl-1H-indol-6-yl)-quinoxalin-6-yl]-(4-methyl-morpholin-2-ylmethyl)-amine (35.0 mg; yield 53%; 99% by HPLC) is obtained as a yellow powder.

### Example 49

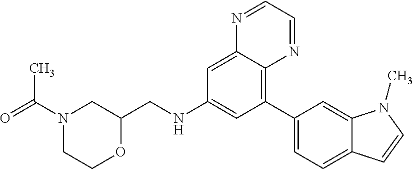 [SMILES: CC(=O)N1CCOC(CNc2cc(-c3ccc4ccn(C)c4c3)c3nccnc3c2)C1]

The product is prepared according to General Procedure 6, described for Intermediate 6 with 7-chloro-5-(1-methyl-1H-indol-6-yl)-quinoxaline (Intermediate 4) (60.0 mg, 0.20 mmol; 1.0 eq.), [1-(4-acetyl-2-morpholinyl)methyl]amine*2 HCl (113.30 mg; 0.49 mmol; 2.40 eq.), Cs₂CO₃ (532.40 mg; 1.63 mmol; 8.00 eq.), Pd(OAc)₂ (2.29 mg; 0.01 mmol; 0.05 eq.) and BINAP (10.17 mg; 0.02 mmol; 0.08 eq.). Purification by FCC (DCM/MeOH; gradient). 1-(2-{[8-(1-Methyl-1H-indol-6-yl)quinoxalin-6-ylamino]methyl}morpholin-4-yl)ethanone (32.00 mg; yield 36%; 94% by HPLC) is obtained as a yellow solid.

### Example 50

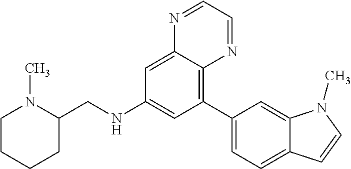 [SMILES: CN1CCCCC1CNc1cc(-c2ccc3ccn(C)c3c2)c2nccnc2c1]

The product is prepared according to General Procedure 5, described in Example 30 with 7-chloro-5-(1-methyl-1H-indol-6-yl)-quinoxaline (Intermediate 4) (75.00 mg; 0.26 mmol; 1.00 eq.), C-(1-methylpiperidin-2-yl)-methylamine (40.92 mg; 0.32 mmol; 1.25 eq.), NaOtBu (34.35 mg; 0.36 mmol; 1.40 eq.), [(Cinnamyl)PdCl]₂ (6.61 mg; 0.01 mmol; 0.05 eq.), BippyPhos (10.35 mg; 0.02 mmol; 0.08 eq.) and toluene (5.00 mL). Purification by FCC (EtOAc/DCM/MeOH; gradient). [8-(1-Methyl-1H-indol-6-yl)quinoxalin-6-yl]-(1-methylpiperidin-2-ylmethyl)amine (52.00 mg; yield 51%; 97% by HPLC) is obtained as a yellow solid.

### Example 51

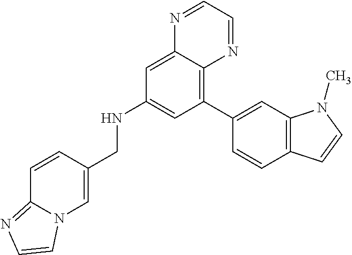 [SMILES: Cn1ccc2ccc(-c3cc(NCc4ccc5nccn5c4)cc4nccnc34)cc21]

The product is prepared according to General Procedure 6, described for Intermediate 6 with 7-chloro-5-(1-methyl-1H-indol-6-yl)-quinoxaline (Intermediate 4) (50.00 mg; 0.17 mmol; 1.00 eq.), C-imidazo[1,2-a]pyridin-6-yl-methylamine (38.34 mg; 0.26 mmol; 1.50 eq.), Cs₂CO₃ (168.06 mg; 0.51 mmol; 3.00 eq.), BINAP (10.81 mg; 0.02 mmol; 0.10 eq.), Pd(OAc)₂ (4.02 mg; 0.02 mmol; 0.10 eq.) and 1,4-dioxane (2.00 mL). Purification by FCC (DCM/MeOH: gradient). Imidazo[1,2-a]pyridin-6-ylmethyl-[8-(1-methyl-1H-indol-6-yl)-quinoxalin-6-yl]-amine (38.00 mg; yield 54%; 97% by HPLC) is obtained as a yellow powder.

### Example 52

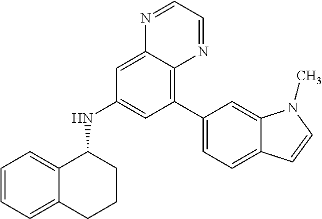 [SMILES: Cn1ccc2ccc(-c3cc(N[C@@H]4CCCc5ccccc54)cc4nccnc34)cc21]

The product is prepared according to General Procedure 2, described in Example 1 with 7-chloro-5-(1-methyl-1H-indol-6-yl)-quinoxaline (Intermediate 4) (33.00 mg, 0.11 mmol; 1.0 eq.), (R)-(1,2,3,4-tetrahydronaphthalen-1-yl)amine (0.04 mL; 0.25 mmol; 2.40 eq.), NaOtBu (30.45 mg; 0.32 mmol; 3.00 eq.), BINAP (13.15 mg; 0.02 mmol; 0.20 eq.), Pd₂(dba)₃ (10.00 mg; 0.0 mmol; 0.10 eq.) and toluene (4.0 mL). Purification by FCC (hexane/EtOAc; gradient). 8-(1-methyl-1H-indol-6-yl)-N-[(1R)-1,2,3,4-tetrahydronaphthalen-1-yl]quinoxalin-6-amine (27.00 mg; yield 60%; 96% by HPLC) is obtained as a yellow powder.

### Example 53

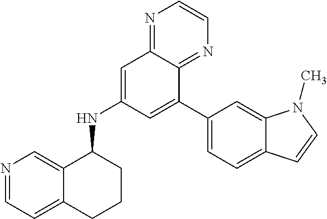 [SMILES: Cn1ccc2ccc(-c3cc(N[C@H]4CCCc5ccncc54)cc4nccnc34)cc21]

The product is prepared according to General Procedure 2, described in Example 1 with 7-chloro-5-(1-methyl-1H-indol-6-yl)-quinoxaline (Intermediate 4) (60.00 mg; 0.20 mmol; 1.00 eq.), (S)-(5,6,7,8-tetrahydroisoquinolin-8-yl)amine hydrochloride (65.72 mg; 0.30 mmol; 1.50 eq.), NaOtBu (38.08 mg; 0.40 mmol; 2.00 eq.), BINAP (24.67 mg; 0.04 mmol; 0.20 eq.), Pd₂(dba)₃ (18.14 mg; 0.02 mmol; 0.10 eq.) and toluene (1.00 mL). Purification by FCC (hexane/EtOAc; gradient). Repurification by preparative HPLC. 8-(1-Methyl-1H-indol-6-yl)-N-[(8S)-5,6,7,8-tetrahydroisoquinolin-8-yl]quinoxalin-6-amine (23.00 mg; yield 28%; 98% by HPLC) is obtained as a yellow powder.

### Example 54

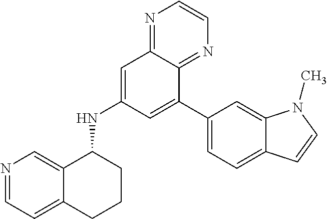 [SMILES: Cn1ccc2ccc(-c3cc(N[C@@H]4CCCc5ccncc54)cc4nccnc34)cc21]

The product is prepared according to General Procedure 2, described in Example 1 with 7-chloro-5-(1-methyl-1H-indol-6-yl)-quinoxaline (Intermediate 4) (60.00 mg; 0.20 mmol; 1.00 eq.), (R)-(5,6,7,8-tetrahydroisoquinolin-8-yl)amine hydrochloride (65.72 mg; 0.30 mmol; 1.50 eq.), NaOtBu (76.16 mg; 0.79 mmol; 4.00 eq.), BINAP (12.34 mg; 0.02 mmol; 0.10 eq.), Pd₂(dba)₃ (9.07 mg; 0.01 mmol; 0.05 eq.) and toluene (3.00 mL). Purification by FCC (hexane/EtOAc; gradient). 8-(1-Methyl-1H-indol-6-yl)-N-[(8R)-5,6,7,8-tetrahydroisoquinolin-8-yl]quinoxalin-6-amine (53.00 mg; yield 64%; 97% by HPLC) is obtained as a yellow powder.

### Example 55

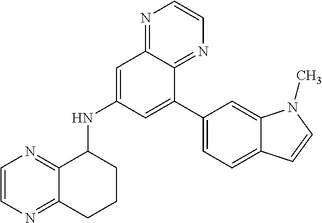 [SMILES: Cn1ccc2ccc(-c3cc(NC4CCCc5nccnc54)cc4nccnc34)cc21]

The product is prepared according to General Procedure 2, described in Example 1 with 7-chloro-5-(1-methyl-1H-indol-6-yl)-quinoxaline (Intermediate 4) (50.00 mg; 0.17 mmol; 1.00 eq.), 5,6,7,8-Tetrahydro-quinoxalin-5-ylamine (0.04 mL; 0.34 mmol; 2.00 eq.), NaOtBu (49.07 mg; 0.51 mmol; 3.00 eq.), BINAP (21.20 mg; 0.03 mmol; 0.20 eq.), Pd₂(dba)₃ (15.59 mg; 0.02 mmol; 0.10 eq.) and toluene (2.00 mL). Purification by FCC (DCM/MeOH; gradient), repurification by preparative HPLC. [8-(1-Methyl-1H-indol-6-yl)-quinoxalin-6-yl]-(5,6,7,8-tetrahydro-quinoxalin-5-yl)-amine (20.00 mg; yield 28%; 99% by HPLC) is obtained as a yellow powder.

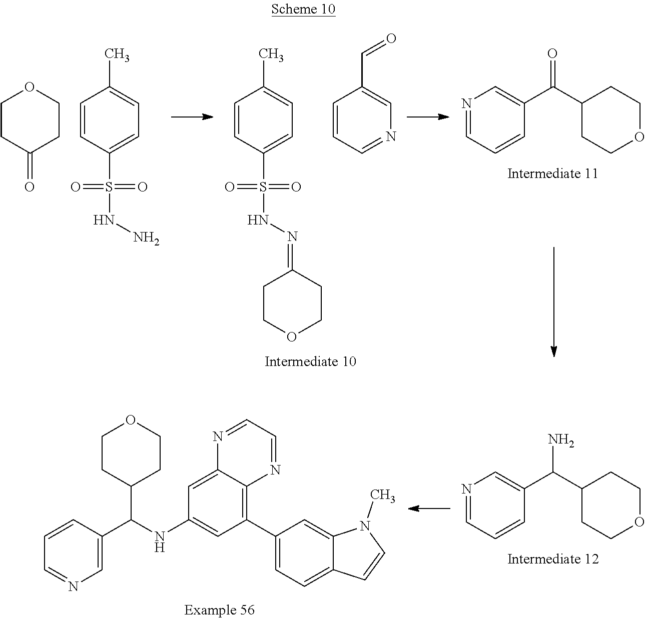

### Intermediate 10—General Procedure 12

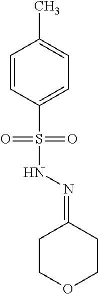 [SMILES: Cc1ccc(S(=O)(=O)NN=C2CCOCC2)cc1]

A round bottom flask is charged with suspension of 4-methylbenzenesulfonohydrazide (0.87 mL; 3.00 mmol; 1.00 eq.) in MeOH (6.00 mL) under argon atmosphere. After stirring for 5 min tetrahydropyran-4-one (0.28 mL; 3.00 mmol; 1.00 eq.) is added. Clear solution is stirred at rt for 3h. After this time, solvent is evaporated to afford 4-methyl-N-(tetrahydropyran-4-ylideneamino)-benzenesulfonamide (874.20 mg; yield 108.7%; 100% by UPLC).

### Intermediate 11 General Procedure 13

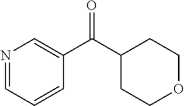 [SMILES: O=C(c1cccnc1)C1CCOCC1]

A sealed tube is charged with 4-methyl-N-(tetrahydropyran-4-ylideneamino)benzenesulfonamide (Intermediate 10) (874.00 mg; 3.26 mmol; 1.00 eq.) and Cs₂CO₃ (1591.87 mg; 4.89 mmol; 1.50 eq.). The tube is sealed and solids are purged with argon and then pyridine-3-carbaldehyde (348.87 mg; 3.26 mmol; 1.00 eq.) and 1,4-dioxane (12.00 mL) are added. RM is stirred at 110° C. for 18 h. After this time, the mixture is quenched with solution of NH₄Cl in water, extracted with DCM. Organic phase is washed with brine, dried over Na₂SO₄ and then solvent is evaporated. Crude product is purified by FCC (hexane/EtOAc; gradient) to afford pyridin-3-yl-(tetrahydro-pyran-4-yl)-methanone (241.90 mg; yield 36%; 94% by UPLC).

### Intermediate 12 General Procedure 14

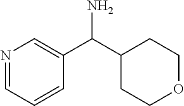 [SMILES: NC(c1cccnc1)C1CCOCC1]

To a solution of pyridin-3-yl-(tetrahydropyran-4-yl)-methanone (Intermediate 11) (100.00 mg; 0.49 mmol; 1.00 eq.) in 7M NH₃ in MeOH (1.00 mL), TTIP (0.29 mL; 0.98 mmol; 2.00 eq.) is added. RM is stirred at 60° C. overnight. Aftert this time solution is cooled to 0° C. and NaBH₄ (74.39 mg; 1.97 mmol; 4.00 eq.) is added. RM is stirred at rt for 3 h and then water is added. RM is extracted with EtOAc. The organic layer is washed with water and brine, dried over anhydrous Na₂SO₄, filtered and evaporated. C-Pyridin-3-yl-C-(tetrahydro-pyran-4-yl)-methylamine (100.40 mg; yield 90%; 85% by UPLC) is directly used in the next step without further purification.

### Example 56

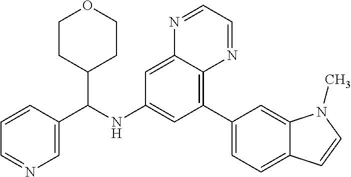 [SMILES: Cn1ccc2ccc(-c3cc(NC(c4cccnc4)C4CCOCC4)cc4nccnc34)cc21]

The product is prepared according to General Procedure 3, described in Example 18 with 7-chloro-5-(1-methyl-1H-indol-6-yl)-quinoxaline (Intermediate 4) (50.00 mg; 0.17 mmol; 1.00 eq.), C-pyridin-3-yl-C-(tetrahydro-pyran-4-yl)-methylamine (Intermediate 12) (36.57 mg; 0.16 mmol; 0.95 eq.), BrettPhos (3.65 mg; 0.01 mmol; 0.04 eq.), BrettPhos precatalyst (5.44 mg; 0.01 mmol; 0.04 eq.) and LiHMDS 1.0 M in THF (272.34 μl; 0.27 mmol; 1.60 eq.). Purification by FCC (hexane/EtOAc; gradient). Repurification by preparative HPLC. [8-(1-Methyl-1H-indol-6-yl)-quinoxalin-6-yl]-[pyridin-3-yl-(tetrahydro-pyran-4-yl)-methyl]-amine (4.50 mg; yield 6%; 97% by HPLC) is obtained as a yellow powder.

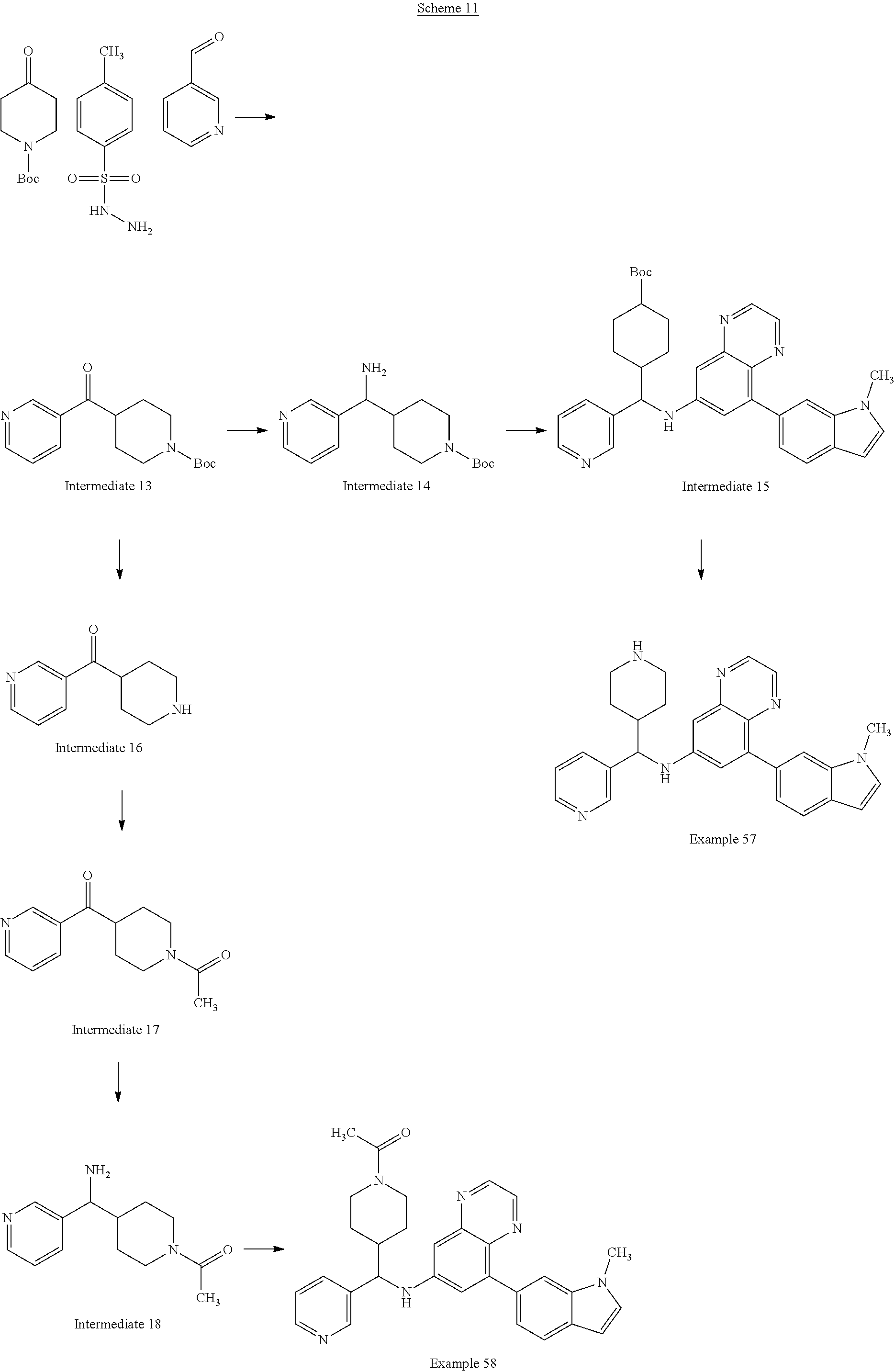

### Intermediate 13 General Procedure 15

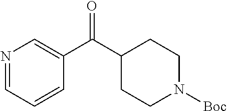 [SMILES: CC(C)(C)OC(=O)N1CCC(C(=O)c2cccnc2)CC1]

A sealed tube is charged with 4-oxopiperidine-1-carboxylic acid tert-butyl ester (200.00 mg; 1.00 mmol; 1.00 eq.), 4-methylbenzenesulfonohydrazide (186.94 mg; 1.00 mmol; 1.00 eq.) and MeOH (3.00 mL). RM is stirred at rt for 3 h. After this time, solvent is evaporated and residue is dissolved in 1,4-dioxane (3.00 mL). Then Cs₂CO₃ (245.29 mg; 0.75 mmol; 0.75 eq.) and pyridine-3-carbaldehyde (107.51 mg; 1.00 mmol; 1.00 eq.) are added and RM is stirred at 120° C. for 6 h. Solvent is evaporated and oily residue is purified by FCC (hexane/EtOAc; gradient; silica gel deactivated with NH₃). 4-(Pyridine-4-carbonyl)-piperidine-1-carboxylic acid tert-butyl ester (145.50 mg; yield 46%; 92% by UPLC) is obtained as a colorless oil.

### Intermediate 14

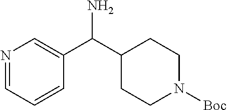 [SMILES: CC(C)(C)OC(=O)N1CCC(C(N)c2cccnc2)CC1]

The product is prepared according to General Procedure 14, described for Intermediate 12 with 4-(pyridine-4-carbonyl)-piperidine-1-carboxylic acid tert-butyl ester (Intermediate 13) (88.00 mg; 0.28 mmol; 1.00 eq.), TTIP (0.17 mL; 0.56 mmol; 2.00 eq.), NaBH₄ (42.52 mg; 1.12 mmol; 4.00 eq.) and 7M NH₃ in MeOH (1.00 mL). After extraction 4-(aminopyridin-3-yl-methyl)-piperidine-1-carboxylic acid tert-butyl ester (113.00 mg; yield 100%, 73% by UPLC) is directly used in the next step.

### Intermediate 15

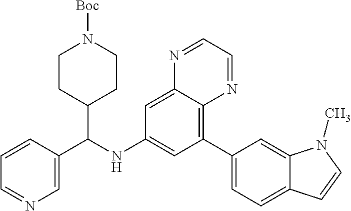 [SMILES: Cn1ccc2ccc(-c3cc(NC(c4cccnc4)C4CCN(C(=O)OC(C)(C)C)CC4)cc4nccnc34)cc21]

The product is prepared according to General Procedure 2, described in Example 1 with 7-chloro-5-(1-methyl-1H-indol-6-yl)-quinoxaline (Intermediate 4) (37.00 mg; 0.13 mmol; 1.00 eq.), 4-(aminopyridin-3-ylmethyl)-piperidine-1-carboxylic acid tert-butyl ester (Intermediate 14) (75.42 mg; 0.19 mmol; 1.50 eq.), NaOtBu (42.37 mg; 0.44 mmol; 3.50 eq.), BINAP (15.69 mg; 0.03 mmol; 0.20 eq.), Pd₂(dba)₃ (11.53 mg; 0.01 mmol; 0.10 eq.) and toluene (3.00 mL). Purification by FCC (DCM/MeOH; gradient). 4-{[8-(1-Methyl-1H-indol-6-yl)-quinoxalin-6-ylamino]-pyridin-3-yl-methyl}-piperidine-1-carboxylic acid tert-butyl ester (64.10 mg; yield 87%; 94% by UPLC) is obtained as a yellow amorphous powder.

### Example 57

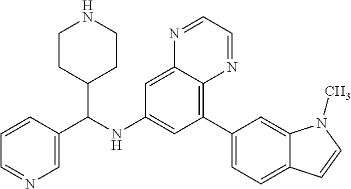 [SMILES: Cn1ccc2ccc(-c3cc(NC(c4cccnc4)C4CCNCC4)cc4nccnc34)cc21]

The product is prepared according to General Procedure 11, described in Example 46 with 4-{[8-(1-methyl-1H-indol-6-yl)-quinoxalin-6-ylamino]-pyridin-3-yl-methyl}-piperidine-1-carboxylic acid tert-butyl ester (Intermediate 15) (32.00 mg; 0.05 mmol; 1.00 eq.), 2M HCl in Et₂O (3.00 mL; 6.00 mmol; 109.44 eq.) and DCM (1.00 mL). The precipitated product is purified by preparative HPLC. [8-(1-Methyl-1H-indol-6-yl)-quinoxalin-6-yl]-(piperidin-4-yl-pyridin-3-yl-methyl)-amine (6.00 mg; yield 19%; 80% by HPLC) is obtained as a yellow powder.

### Intermediate 16

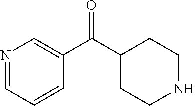 [SMILES: O=C(c1cccnc1)C1CCNCC1]

The product is prepared according to General Procedure 11, described in Example 46 with 4-(pyridine-4-carbonyl)-piperidine-1-carboxylic acid tert-butyl ester (Intermediate 13), TFA (3.00 mL) and DCM (1.00 mL). Purification by FCC (NH₂ column; DCM/MeOH; gradient). Piperidin-4-yl-pyridin-3-yl-methanone (71.00 mg; yield 62%, 100% by UPLC) is obtained as a white powder.

### Intermediate 17—General Procedure 16

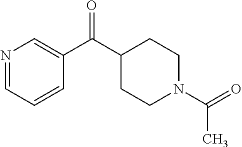 [SMILES: CC(=O)N1CCC(C(=O)c2cccnc2)CC1]

A piperidin-4-yl-pyridin-3-yl-methanone (Intermediate 16) (71.00 mg; 0.37 mmol; 1.00 eq.) is added to mixture of anhydrous DCM (2.00 mL) and TEA (0.12 mL; 0.93 mmol; 2.50 eq.). Acetic anhydride (0.04 mL; 0.41 mmol; 1.10 eq.) is added portionwise at 0° C. and resulted mixture is stirred at rt overnight. The reaction is quenched with water, extracted with n-BuOH. Organic layer is washed with brine, dried over Na₂SO₄, filtered and concentrated in vacuo. Crude 1-[4-(pyridine-3-carbonyl)-piperidin-1-yl]-ethanone (70.70 mg; yield 71%; 87% by UPLC) is used in the next step.

### Intermediate 18

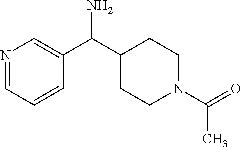 [SMILES: CC(=O)N1CCC(C(N)c2cccnc2)CC1]

The product is prepared according to General Procedure 14, described for Intermediate 12 with 1-[4-(pyridine-3-carbonyl)-piperidin-1-yl]-ethanone (Intermediate 17) (70.70 mg; 0.26 mmol; 1.00 eq.), TTIP (0.16 mL; 0.53 mmol; 2.00 eq.), NaBH₄ (40.07 mg; 1.06 mmol; 4.00 eq.) and 7M NH₃ in MeOH (1.00 mL). Crude 1-[4-(aminopyridin-3-yl-methyl)-piperidin-1-yl]-ethanone (128.00 mg; yield 190.6%; 92% by UPLC) is directly used in the next step without further purification.

### Example 58

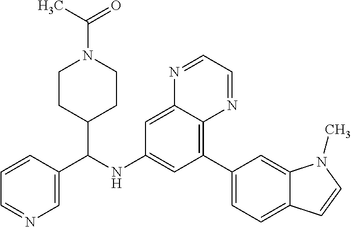 [SMILES: CC(=O)N1CCC(C(Nc2cc(-c3ccc4ccn(C)c4c3)c3nccnc3c2)c2cccnc2)CC1]

The product is prepared according to General Procedure 2, described in Example 1 with 7-chloro-5-(1-methyl-1H-indol-6-yl)-quinoxaline (Intermediate 4) (30.00 mg; 0.10 mmol; 1.00 eq.), 1-[4-(aminopyridin-3-yl-methyl)-piperidin-1-yl]-ethanone (Intermediate 18) (38.46 mg; 0.15 mmol; 1.50 eq.), NaOtBu (34.01 mg; 0.35 mmol; 3.50 eq.), BINAP (12.59 mg; 0.02 mmol; 0.20 eq.), Pd₂(dba)₃ (9.26 mg; 0.01 mmol; 0.10 eq.) and toluene (3.00 mL). Purification by FCC (DCM/MeOH; gradient). 1-(4-{[8-(1-Methyl-1H-indol-6-yl)-quinoxalin-6-ylamino]-pyridin-3-yl-methyl}-piperidin-1-yl)-ethanone (10.00 mg; yield 19%; 94% by HPLC) is obtained as a yellow powder.

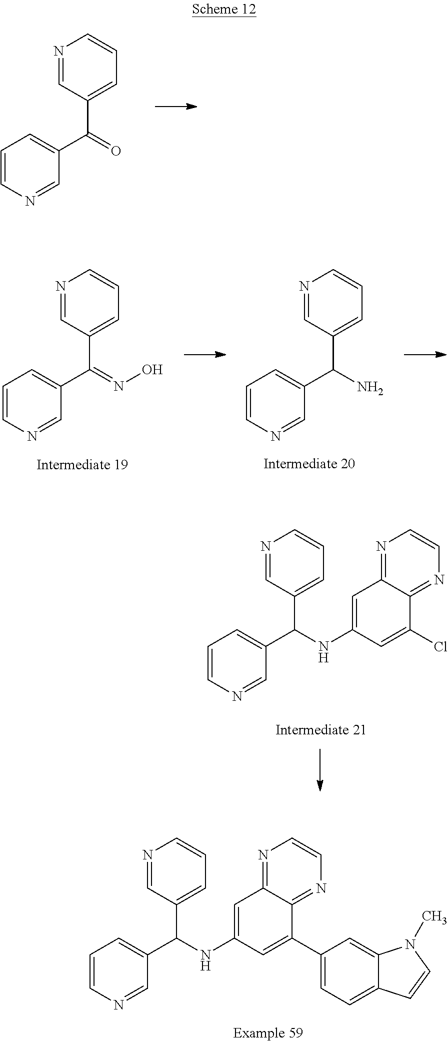 [SMILES: O=C(c1cccnc1)c1cccnc1 ; ON=C(c1cccnc1)c1cccnc1 ; NC(c1cccnc1)c1cccnc1 ; Clc1cc(NC(c2cccnc2)c2cccnc2)cc2nccnc12 ; Cn1ccc2ccc(-c3cc(NC(c4cccnc4)c4cccnc4)cc4nccnc34)cc21]

### Intermediate 19—General Procedure 17

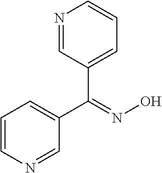 [SMILES: ON=C(c1cccnc1)c1cccnc1]

A solution of dipyridin-3-yl-methanone (200.00 mg; 1.09 mmol; 1.00 eq.) in anhydrous MeOH is added to the well stirred solution of NaOAc (222.68 mg; 2.71 mmol; 2.50 eq.) and hydroxylamine hydrochloride (188.63 mg; 2.71 mmol; 2.50 eq.) in anhydrous MeOH. RM is refluxed under argon atmosphere for 2 h. After this time, solvent is evaporated and the residue is extracted with water and EtOAc. Organic layer is washed with brine, dried over Na₂SO₄, filtered and evaporated. Crude dipyridin-3-yl-methanone oxime (216.00 mg; yield 99%; 99% by HPLC) is used in the next step without purification.

### Intermediate 20—General Procedure 18

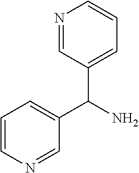 [SMILES: NC(c1cccnc1)c1cccnc1]

Dipyridin-3-yl-methanone oxime (Intermediate 19) (216.30 mg; 1.09 mmol; 1.00 eq.) and NH₄OAc (125.54 mg; 1.63 mmol; 1.50 eq.) are dissolved in EtOH (5.00 mL), water (5.00 mL) and ammonia 28% (5.00 mL). The mixture is heated to 80° C. and zinc dust (355.01 mg; 5.43 mmol; 5.00 eq.) is added over a period of 1 h. RM is then stirred at 80° C. overnight. After this time, the mixture is filtered through a Celite® pad and the filtrate is diluted with EtOAc and extracted with water. Organic phase is washed with brine, dried over Na₂SO₄ and then solvent is evaporated. Crude product is purified by FCC (DCM/MeOH; gradient). C,C-Di-pyridin-3-yl-methylamine (136.00 mg; yield 67%; 99% by UPLC) is obtained as a white solid.

### Intermediate 21

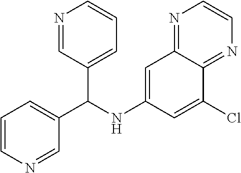 [SMILES: Clc1cc(NC(c2cccnc2)c2cccnc2)cc2nccnc12]

The product is prepared according to General Procedure 2, described in Example 1 with C,C-dipyridin-3-ylmethylamine (Intermediate 20) (0.13 mL; 0.46 mmol; 1.00 eq.), 7-bromo-5-chloroquinoxaline (Intermediate 3) (101.51 mg; 0.42 mmol; 0.90 eq.), NaOtBu (111.29 mg; 1.16 mmol; 2.50 eq.), BINAP (51.92 mg; 0.08 mmol; 0.18 eq.), Pd₂(dba)₃ (42.42 mg; 0.05 mmol; 0.10 eq.) and toluene (3.00 mL). Purification by FCC (hexane/EtOAc; gradient). N-[Bis(pyridin-3-yl)methyl]-8-chloroquinoxalin-6-amine (130.00 mg; yield 80.0%; 99% by HPLC) is obtained as an orange powder.

### Example 59

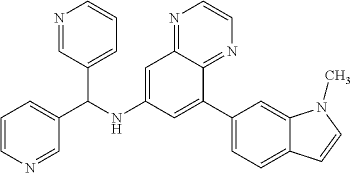 [SMILES: Cn1ccc2ccc(-c3cc(NC(c4cccnc4)c4cccnc4)cc4nccnc34)cc21]

The product is prepared according to General Procedure 1, described for Intermediate 4 with (8-chloroquinoxalin-6-yl)-(dipyridin-3-yl-methyl)-amine (Intermediate 21) (100.00 mg; 0.29 mmol; 1.00 eq.), 1-methyl-6-(4,4,5,5-tetramethyl-[1,3,2]dioxaborolan-2-yl)-1H-indole (95.40 mg; 0.35 mmol; 1.20 eq.), DIPEA (0.10 mL; 0.58 mmol; 2.00 eq.), Pd(dppf)Cl₂ (21.04 mg; 0.03 mmol; 0.10 eq.), 1,4-dioxane (3.00 mL) and water (3.00 mL). Purification by FCC (hexane/EtOAc; gradient). N-[Bis(pyridin-3-yl)methyl]-8-(1-methyl-1H-indol-6-yl)quinoxalin-6-amine (35.00 mg; yield 27%; 98% by HPLC) is obtained as a yellow powder.

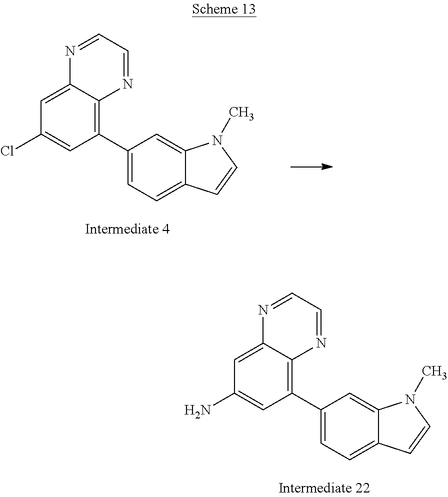 [SMILES: Cn1ccc2ccc(-c3cc(Cl)cc4nccnc34)cc21 ; Cn1ccc2ccc(-c3cc(N)cc4nccnc34)cc21]

### Intermediate 22—General Procedure 19

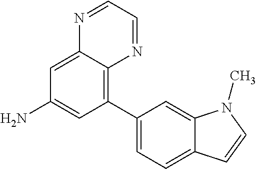 [SMILES: Cn1ccc2ccc(-c3cc(N)cc4nccnc34)cc21]

A sealed tube equipped with a stir bar is charged with 7-chloro-5-(1-methyl-1H-indol-6-yl)-quinoxaline (Intermediate 4) (100.00 mg; 0.31 mmol; 1.00 eq.), Pd₂(dba)₃ (28.84 mg; 0.03 mmol; 0.10 eq.), Me₄tBuXPhos (15.14 mg; 0.03 mmol; 0.10 eq.) and NaOtBu (42.37 mg; 0.44 mmol; 1.40 eq.). The tube is then sealed, evacuated and backfilled with argon (three times). Ammonia solution 0.5 M in dioxane (12.60 mL; 6.30 mmol; 20.00 eq.) is added into the tube via syringe. RM is then stirred at 80° C. for 5 h. After this time, the mixture is filtered through a Celite® pad and the filtrate is extracted with EtOAc and water. The combined organic layers are washed with brine, dried over Na₂SO₄ and concentrated in vacuo. The residue is purified by FCC (DCM:MeOH; gradient). 8-(1-Methyl-1H-indol-6-yl)quinoxalin-6-amine (70.00 mg; yield 78%; 96% by HPLC) is obtained as a yellow powder.

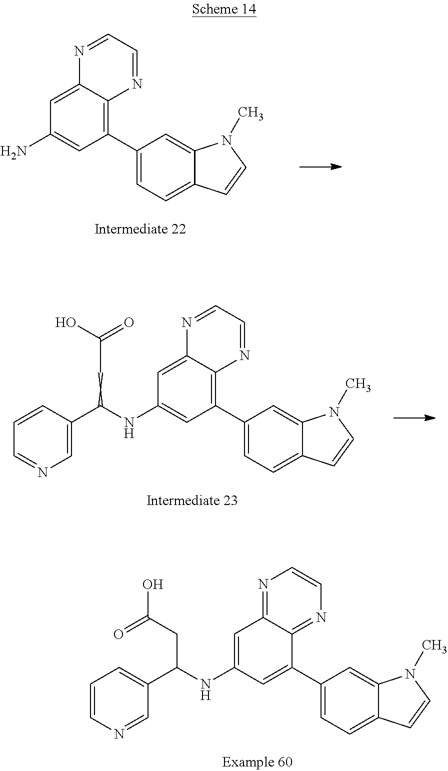 [SMILES: Cn1ccc2ccc(-c3cc(N)cc4nccnc34)cc21 ; Cn1ccc2ccc(-c3cc(NC(=CC(=O)O)c4cccnc4)cc4nccnc34)cc21 ; Cn1ccc2ccc(-c3cc(NC(CC(=O)O)c4cccnc4)cc4nccnc34)cc21]

### Intermediate 23—General Procedure 20

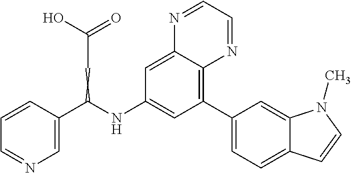 [SMILES: Cn1ccc2ccc(-c3cc(NC(=CC(=O)O)c4cccnc4)cc4nccnc34)cc21]

To a mixture of 8-(1-methyl-1H-indol-6-yl)-quinoxalin-6-ylamine (Intermediate 22) (200.00 mg; 0.73 mmol; 1.00 eq.), 3-oxo-3-pyridin-3-yl-propionic acid ethyl ester (0.15 mL; 0.80 mmol; 1.10 eq.), PTSA (12.68 mg; 0.07 mmol; 0.10 eq.) in toluene (5.00 mL) are added freshly dried molecular sieves. RM is heated at 110° C. overnight. After this time, the mixture is filtered through a Celite® pad and the filtrate is evaporated. The crude product is purified by FCC (DCM/MeOH; gradient). Repurification by preparative HPLC and after evaporation extraction with saturated NaHCO₃ solution is done. (E/Z)-3-{[8-(1-Methyl-1H-indol-6-yl)quinoxalin-6-yl]amino}-3-(pyridin-3-yl)prop-2-enoic acid (55.00 mg; yield 18%; 98% by HPLC) is obtained as a yellow powder (mixture of cis/trans isomers).

### Example 60—General Procedure 21

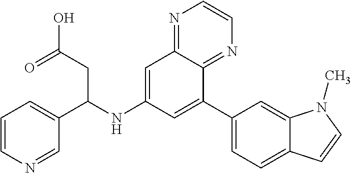 [SMILES: Cn1ccc2ccc(-c3cc(NC(CC(=O)O)c4cccnc4)cc4nccnc34)cc21]

To a solution of (E/Z)-3-[8-(1-methyl-1H-indol-6-yl)-quinoxalin-6-ylamino]-3-pyridin-3-yl-acrylic acid (Intermediate 23) (65.00 mg; 0.15 mmol; 1.00 eq.) in THF (3.00 mL) is added CH₃COOH (0.50 mL). Then RM is stirred at rt for 2h and then NaBH(OAc)₃ (102.73 mg; 0.46 mmol; 3.00 eq.) is added. The mixture reaction is stirred at rt overnight and then RM is evaporated. The residue is extracted with EtOAc and water, washed with NaHCO₃, dried over Na₂SO₄ and concentrated. Crude product is purified by preparative HPLC. 3-{[8-(1-methyl-1H-indol-6-yl)quinoxalin-6-yl]amino}-3-(pyridin-3-yl)propanoic acid (30.00 mg; yield 46%; 99% by HPLC) is obtained as a red solid.

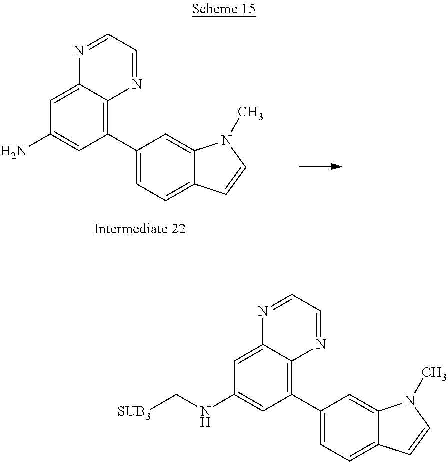

### Example 61—General Procedure 22

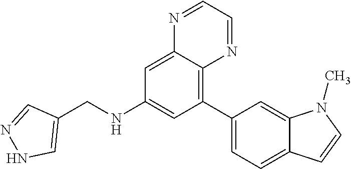 [SMILES: Cn1ccc2ccc(-c3cc(NCc4cn[nH]c4)cc4nccnc34)cc21]

A mixture of 8-(1-methyl-1H-indol-6-yl)-quinoxalin-6-ylamine (Intermediate 22) (100.00 mg; 0.34 mmol; 1.00 eq.), 1H-pyrazole-4-carbaldehyde (41.89 mg; 0.44 mmol; 1.30 eq.) and CH₃COOH (0.10 mL; 1.75 mmol; 5.22 eq.) in 1,2-dichloroethane (5.00 mL) under argon at 5° C. is stirred for 10 min and next 1h at rt. After this time, RM is cooled to 5° C. and NaBH(OAc)₃ (96.80 mg; 0.44 mmol; 1.30 eq.) is added and then RM is stirred at rt overnight. RM is diluted with water and extracted with EtOAc. Combined organic layers are washed with brine, dried over Na₂SO₄, filtered and evaporated. Brown residue is purified by FCC (DCM/MeOH; gradient) and repurified by preparative HPLC. 8-(1-methyl-1H-indol-6-yl)-N-(1H-pyrazol-4-ylmethyl)quinoxalin-6-amine (55.00 mg; yield 46%; 99% by HPLC) as a yellow solid.

### Example 62

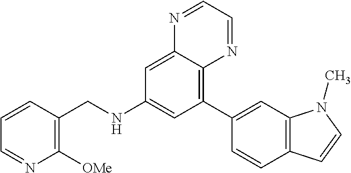 [SMILES: COc1ncccc1CNc1cc(-c2ccc3ccn(C)c3c2)c2nccnc2c1]

The product is prepared according to General Procedure 22, described in Example 61 with 8-(1-methyl-1H-indol-6-yl)-quinoxalin-6-ylamine (Intermediate 22) (100.00 mg; 0.34 mmol; 1.00 eq.), 2-methoxypyridine-3-carbaldehyde (51.50 μl; 0.44 mmol; 1.30 eq.), NaBH(OAc)₃ (96.80 mg; 0.44 mmol; 1.30 eq.), CH₃COOH (100.18 μl; 1.75 mmol; 5.22 eq.) and 1,2-dichloroethane (5.00 mL). Purification by FCC (hexane/EtOAc; gradient). N-[(2-Methoxypyridin-3-yl)methyl]-8-(1-methyl-1H-indol-6-yl)quinoxalin-6-amine (44.00 mg; yield 32%; 95% by HPLC) is obtained as a brown powder.

### Example 63—General Procedure 23

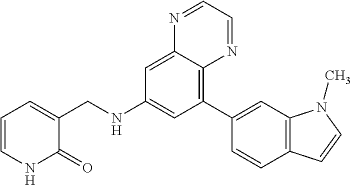 [SMILES: Cn1ccc2ccc(-c3cc(NCc4ccc[nH]c4=O)cc4nccnc34)cc21]

To a stirred solution of 8-(1-methyl-1H-indol-6-yl)-quinoxalin-6-ylamine (Intermediate 22) (50.00 mg; 0.18 mmol; 1.00 eq.) and 2-oxo-1,2-dihydropyridine-3-carbaldehyde (22.21 mg; 0.18 mmol; 1.00 eq.) in anhydrous DCM (4.00 mL), Hantzsch ester (57.13 mg; 0.23 mmol; 1.25 eq.) and TMCS (4.58 μl; 0.04 mmol; 0.20 eq.) are added in one portion under argon. RM is stirred for 18 h at rt, then poured onto saturated aqueous NaHCO₃ solution and extracted with DCM. The organic layer is washed with water, dried over MgSO₄, and concentrated in vacuo. The residue is purified by FCC (hexane/EtOAc; gradient) to give 3-({[8-(1-methyl-1H-indol-6-yl)quinoxalin-6-yl]amino}methyl)-1,2-dihydropyridin-2-one (42.00 mg; yield 60%; 98% by HPLC) as a yellow powder.

### Example 64—General Procedure 24

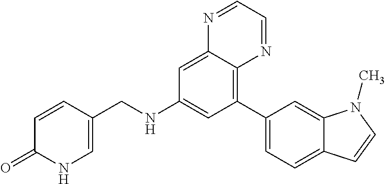 [SMILES: Cn1ccc2ccc(-c3cc(NCc4ccc(=O)[nH]c4)cc4nccnc34)cc21]

To a solution of 8-(1-methyl-1H-indol-6-yl)-quinoxalin-6-ylamine (Intermediate 22) (50.00 mg; 0.18 mmol; 1.00 eq.) and 6-hydroxynicotinaldehyde (44.88 mg; 0.36 mmol; 2.00 eq.) in DCM (5.00 mL), CH₃COOH (0.02 mL; 0.36 mmol; 2.00 eq.) is added. RM is stirred at rt for 1h. Next NaBH(OAc)₃ (122.00 mg; 0.55 mmol; 3.00 eq.) is added. RM is stirred at 40° C. overnight. After extraction, crude product is purified by FCC (DCM/MeOH; gradient). 5-{[8-(1-Methyl-1H-indol-6-yl)quinoxalin-6-ylamino]methyl}pyridin-2-ol (10.00 mg; yield 14%; 97% by HPLC) is obtained as a yellow powder.

### Example 65

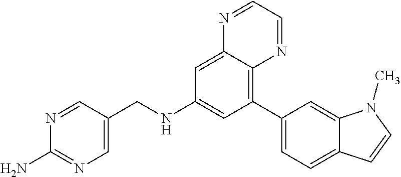 [SMILES: Cn1ccc2ccc(-c3cc(NCc4cnc(N)nc4)cc4nccnc34)cc21]

The product is prepared according to General Procedure 23, described for Example 63 with 8-(1-methyl-1H-indol-6-yl)-quinoxalin-6-ylamine (Intermediate 22) (50.00 mg; 0.17 mmol; 1.00 eq.), 2-Amino-pyrimidine-5-carbaldehyde (22.44 mg; 0.17 mmol; 1.00 eq., Hantzsch ester (57.71 mg; 0.22 mmol; 1.25 eq.), TMCS (4.49 μl; 0.03 mmol; 0.20 eq.) and DCM (3.5 mL). Purification by FCC (DCM/MeOH; gradient). (2-Aminopyrimidin-5-ylmethyl)-[8-(1-methyl-1H-indol-6-yl)-quinoxalin-6-yl]-amine (33.00 mg; yield 48%; 96% by HPLC) is obtained as a yellow powder.

### Example 66

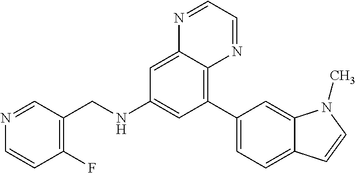 [SMILES: Cn1ccc2ccc(-c3cc(NCc4cnccc4F)cc4nccnc34)cc21]

The product is prepared according to General Procedure 23, described for Example 63 with 8-(1-methyl-1H-indol-6-yl)-quinoxalin-6-ylamine (Intermediate 22) (50.00 mg; 0.18 mmol; 1.00 eq.), 2-fluoropyridine-3-carbaldehyde (0.03 mL; 0.18 mmol; 1.00 eq.), Hantzsch ester (57.71 mg; 0.23 mmol; 1.25 eq.), TMCS (0.01 mL; 0.05 mmol; 0.30 eq.) and DCM (3.0 mL). Purification by FCC (DCM/MeOH; gradient). (2-Fluoropyridin-3-ylmethyl)-[8-(1-methyl-1H-indol-6-yl)-quinoxalin-6-yl]-amine (38.80 mg; yield 53%; 98% by HPLC) is obtained as a bright yellow solid.

### Example 67

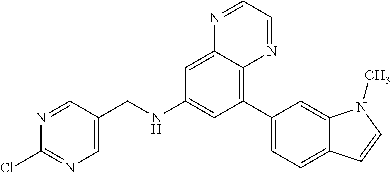 [SMILES: Cn1ccc2ccc(-c3cc(NCc4cnc(Cl)nc4)cc4nccnc34)cc21]

The product is prepared according to General Procedure 23, described for Example 63 with 8-(1-methyl-1H-indol-6-yl)-quinoxalin-6-ylamine (Intermediate 22) (100.00 mg; 0.36 mmol; 1.00 eq.), 2-Chloropyrimidine-5-carbaldehyde (70.40 mg; 0.47 mmol; 1.30 eq.), Hantzsch ester (120.28 mg; 0.45 mmol; 1.25 eq.), TMCS (9.35 μl; 0.07 mmol; 0.20 eq.) and DCE (8.0 mL). Purification by FCC (DCM/MeOH; gradient). (2-Chloro-pyrimidin-5-ylmethyl)-[8-(1-methyl-1H-indol-6-yl)-quinoxalin-6-yl]-amine (119.00 mg; 76% yield; 92% by HPLC) is obtained as a yellow powder.

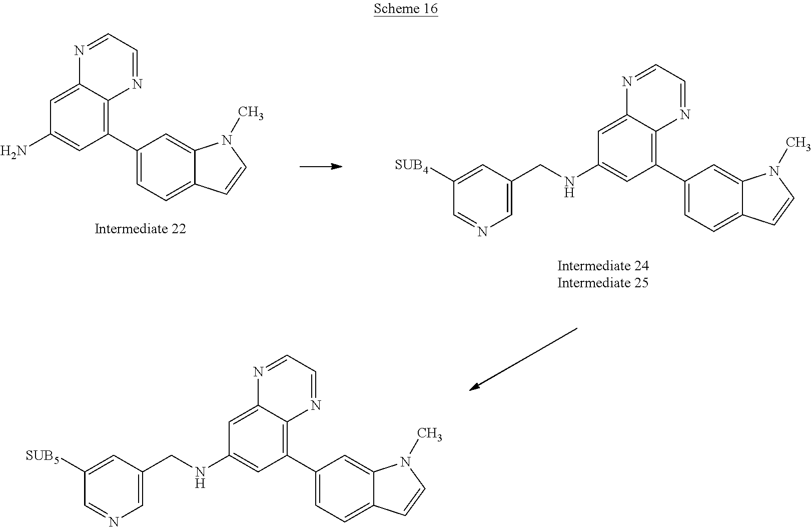

### Intermediate 24

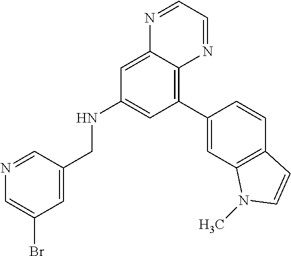 [SMILES: Cn1ccc2ccc(-c3cc(NCc4cncc(Br)c4)cc4nccnc34)cc21]

The product is prepared according to General Procedure 23, described for Example 63 with 8-(1-methyl-1H-indol-6-yl)-quinoxalin-6-ylamine (Intermediate 22) (200.00 mg; 0.72 mmol; 1.00 eq.), 5-bromonicotinaldehyde (134.26 mg; 0.72 mmol; 1.00 eq.), Hantzsch ester (228.53 mg; 0.90 mmol; 1.25 eq.), TMCS (18.32 μl; 0.14 mmol; 0.20 eq.) and anhydrous DCM (3.33 mL). Purification by FCC (DCM/MeOH; gradient). (5-Bromopyridin-3-ylmethyl)-[8-(1-methyl-1H-indol-6-yl)-quinoxalin-6-yl]-amine (250.00 mg; yield 65%; 83% by UPLC) is obtained as a yellow powder.

### Intermediate 25

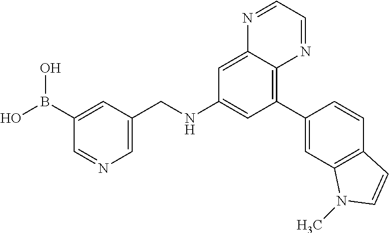 [SMILES: Cn1ccc2ccc(-c3cc(NCc4cncc(B(O)O)c4)cc4nccnc34)cc21]

The product is prepared according to General Procedure 23, described for Example 63 with 8-(1-methyl-1H-indol-6-yl)-quinoxalin-6-ylamine (Intermediate 22) (60.00 mg; 0.20 mmol; 1.00 eq.), 5-(4,4,5,5-tetramethyl-[1,3,2]dioxaborolan-2-yl)-pyridine-3-carbaldehyde (45.78 mg; 0.20 mmol; 1.00 eq.), Hantzsch ester (62.19 mg; 0.25 mmol; 1.25 eq.), TMCS (4.99 μl; 0.04 mmol; 0.20 eq.) and anhydrous DCM (1.00 mL). Purification by FCC (DCM/MeOH; gradient). [5-[[[8-(1-Methylindol-6-yl)quinoxalin-6-yl]amino]methyl]-3-pyridyl]boronic acid (130.00 mg; yield 108.4%; 67% by UPLC) is obtained as a yellow powder.

### Example 68—General Procedure 25

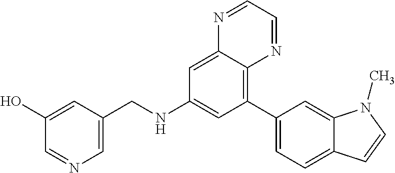 [SMILES: Cn1ccc2ccc(-c3cc(NCc4cncc(O)c4)cc4nccnc34)cc21]

A sealed tube is charged with 5-Bromopyridin-3-ylmethyl)-[8-(1-methyl-1H-indol-6-yl)-quinoxalin-6-yl]-amine (Intermediate 24) (80.00 mg; 0.14 mmol; 1.00 eq.), KOH (17.80 mg; 0.32 mmol; 3.00 eq.), Me₄tBuXPhos (5.09 mg; 0.01 mmol; 0.10 eq.), 1,4-dioxane (1.00 mL) and water (1.00 mL). The suspension is purged with argon and then Pd₂(dba)₃ (12.28 mg; 0.01 mmol; 0.05 eq.) is added. The resulting mixture is heated at 120° C. overnight. After this time, the mixture is diluted with EtOAc and water. The organic layer is washed with water, brine, dried over Na₂SO₄, and concentrated in vacuo. The residue is purified by FCC (hexane/EtOAc: gradient), repurification by preparative HPLC is done. 5-{[8-(1-Methyl-1H-indol-6-yl)-quinoxalin-6-ylamino]-methyl}-pyridin-3-ol (15.00 mg; yield 37%; 98% by HPLC) is obtained as a light orange powder.

### Example 69—General Procedure 26

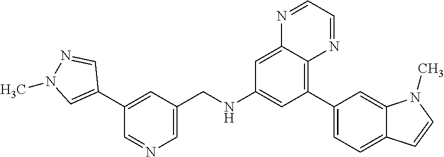 [SMILES: Cn1cc(-c2cncc(CNc3cc(-c4ccc5ccn(C)c5c4)c4nccnc4c3)c2)cn1]

A sealed tube is charged with (5-Bromo-pyridin-3-ylmethyl)-[8-(1-methyl-1H-indol-6-yl)-quinoxalin-6-yl]-amine (Intermediate 24) (80.00 mg; 0.14 mmol; 1.00 eq.), 1-methyl-4-(4,4,5,5-tetramethyl-[1,3,2]dioxaborolan-2-yl)-1H-pyrazole (35.96 mg; 0.17 mmol; 1.20 eq.), 1M Na₂CO₃ (0.22 mL; 0.43 mmol; 3.00 eq.) and DME (1.40 mL). The suspension is purged with argon and then Pd(dppf)Cl₂ (10.54 mg; 0.01 mmol; 0.10 eq.) is added. The resulting mixture is heated at 80° C. for 2 h. After this time, the mixture is diluted with EtOAc and water. The organic layer is washed with water, brine and dried over MgSO₄, and concentrated in vacuo. The residue is purified by FCC (EtOAc/MeOH; gradient). 8-(1-Methyl-1H-indol-6-yl)-N-{[5-(1-methyl-1H-pyrazol-4-yl)pyridin-3-yl]methyl}quinoxalin-6-amine (61.00 mg; yield 91%; 96% by HPLC) is obtained as yellow crystals.

### Example 70—General Procedure 27

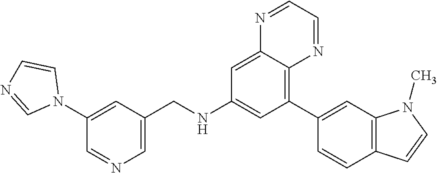 [SMILES: Cn1ccc2ccc(-c3cc(NCc4cncc(-n5ccnc5)c4)cc4nccnc34)cc21]

A sealed tube is charged with K₂CO₃ (39.81 mg; 0.29 mmol; 2.00 eq.), 1H-imidazole (14.71 mg; 0.22 mmol; 1.50 eq.), (5-bromo-pyridin-3-ylmethyl)-[8-(1-methyl-H-indol-6-yl)-quinoxalin-6-yl]-amine (Intermediate 24) (80.00 mg; 0.14 mmol; 1.00 eq.) and DMF (1.00 mL). The suspension is purged with argon and then N,N′-dimethylethylenediamine (7.90 μl; 0.07 mmol; 0.50 eq.) and CuI (13.72 mg; 0.07 mmol; 0.50 eq.) are added and RM is stirred at rt for 30 minutes, and then heated at 110° C. for 16 h. After this time, the mixture is cooled to rt, filtered through a plug of Celite® and extracted with EtOAc. The combined organic layers are washed with saturated brine, dried over MgSO₄ and concentrated in vacuo. The residue is purified by FCC (Hexane/EtOAc; gradient). N-{[5-(1H-imidazol-1-yl)pyridin-3-yl]methyl}-8-(1-methyl-1H-indol-6-yl)quinoxalin-6-amine (36.00 mg; yield 54%; 91% by HPLC) as a yellow powder.

### Example 71—General Procedure 28

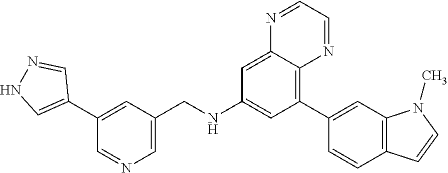 [SMILES: Cn1ccc2ccc(-c3cc(NCc4cncc(-c5cn[nH]c5)c4)cc4nccnc34)cc21]

A sealed tube is charged with (5-bromopyridin-3-ylmethyl)-[8-(1-methyl-1H-indol-6-yl)-quinoxalin-6-yl]-amine (Intermediate 24) (33.00 mg; 0.06 mmol; 1.00 eq.), 4-(4,4,5,5-tetramethyl-[1,3,2]dioxaborolan-2-yl)-1H-pyrazole (25.13 mg; 0.13 mmol; 2.00 eq.), K₂CO₃ (26.85 mg; 0.19 mmol; 3.00 eq.), 1,4-dioxane (1.00 mL) and water (0.50 mL). The suspension is purged with argon and then Pd(PPh₃)₄ (3.74 mg; 0.00 mmol; 0.05 eq.) is added. RM is stirred overnight at 110° C. After this time, the mixture is filtered through a Celite® pad and the filtrate is diluted with EtOAc and extracted with water. Organic phase is washed with brine, dried over Na₂SO₄ and then solvent is evaporated. Crude product is purified by FCC (DCM/MeOH; gradient) to afford 8-(1-methyl-1H-indol-6-yl)-N-{[5-(1H-pyrazol-4-yl)pyridin-3-yl]methyl}quinoxalin-6-amine (11.00 mg; yield 38%; 98% by HPLC) as a yellow solid.

### Example 72—General Procedure 29

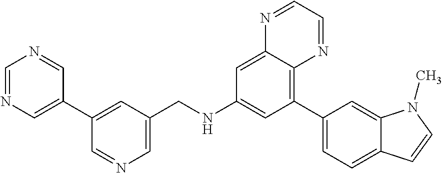 [SMILES: Cn1ccc2ccc(-c3cc(NCc4cncc(-c5cncnc5)c4)cc4nccnc34)cc21]

A microwave tube is charged with (5-bromopyridin-3-ylmethyl)-[8-(1-methyl-1H-indol-6-yl)-quinoxalin-6-yl]-amine (Intermediate 24) (25.00 mg; 0.05 mmol; 1.00 eq.), 5-(4,4,5,5-tetramethyl-[1,3,2]dioxaborolan-2-yl)-pyrimidine (21.10 mg; 0.10 mmol; 2.00 eq.), KOAc (30.15 mg; 0.31 mmol; 6.00 eq.), CH₃CN (1.00 mL) and water (0.50 mL). The suspension is purged with argon and then Pd(dppf)Cl₂ (9.37 mg; 0.01 mmol; 0.25 eq.) is added. Reaction is carried out in a MW reactor at 140° C. for 40 min. After this time, the mixture is filtered through a Celite® pad, the filtrate is evaporated and crude product is purified by FCC (DCM/MeOH; gradient; NH₂ column). 8-(1-methyl-1H-indol-6-yl)-N-{[5-(pyrimidin-5-yl)pyridin-3-yl]methyl}quinoxalin-6-amine (13.00 mg; yield 54%; 95% by HPLC) is obtained as a yellow powder.

### Example 73

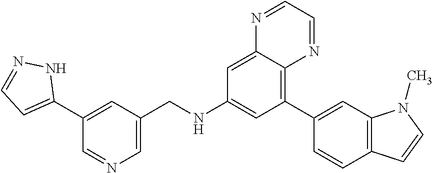 [SMILES: Cn1ccc2ccc(-c3cc(NCc4cncc(-c5ccn[nH]5)c4)cc4nccnc34)cc21]

The product is prepared according to General Procedure 26, described for Example 69 with (5-bromopyridin-3-ylmethyl)-[8-(1-methyl-1H-indol-6-yl)-quinoxalin-6-yl]-amine (Intermediate 24) (50.00 mg; 0.09 mmol; 1.00 eq.), 5-(4,4,5,5-tetramethyl-[1,3,2]dioxaborolan-2-yl)-1H-pyrazole (20.96 mg; 0.11 mmol; 1.20 eq.), 1 M Na₂CO₃ (0.14 mL; 0.27 mmol; 3.00 eq.), Pd(dppf)Cl₂ (6.59 mg; 0.01 mmol; 0.10 eq.) and DME (1.40 mL). Purification by FCC (EtOAc/MeOH; gradient). 8-(1-Methyl-1H-indol-6-yl)-N-{[5-(1H-pyrazol-5-yl)pyridin-3-yl]methyl}quinoxalin-6-amine (10.00 mg; yield 24%; 93% by HPLC) is obtained as a yellow solid.

### Example 74—General Procedure 30

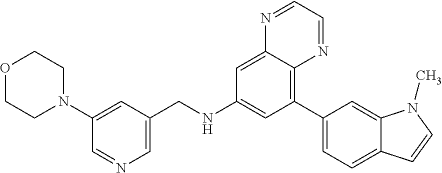 [SMILES: Cn1ccc2ccc(-c3cc(NCc4cncc(N5CCOCC5)c4)cc4nccnc34)cc21]

A sealed tube is charged with (5-bromopyridin-3-ylmethyl)-[8-(1-methyl-1H-indol-6-yl)-quinoxalin-6-yl]-amine (30.00 mg; 0.05 mmol; 1.00 eq.) (Intermediate 24), morpholine (9.09 μl; 0.11 mmol; 2.00 eq.), NaOtBu (7.58 mg; 0.08 mmol; 1.50 eq.) and toluene (3.00 mL). RM is purged with argon and then Xantphos (3.66 mg; 0.0063 mmol; 0.12 eq.) and Pd₂(dba)₃ (1.93 mg; 0.0021 mmol; 0.04 eq.) are added. RM is sealed and heated at 100° C. for 5h. Solvent is evaporated and the residue is purified by preparative HPLC. [8-(1-Methyl-1H-indol-6-yl)-quinoxalin-6-yl]-(5-morpholin-4-yl-pyridin-3-ylmethyl)-amine (3.50 mg; yield 15%; 91% by HPLC) is obtained as a yellow solid.

### Example 75

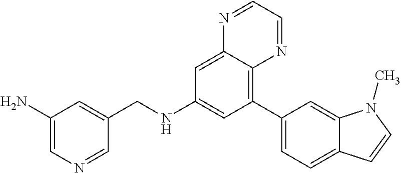 [SMILES: Cn1ccc2ccc(-c3cc(NCc4cncc(N)c4)cc4nccnc34)cc21]

The product is prepared according to General Procedure 19, described for Intermediate 22 (5-bromopyridin-3-ylmethyl)-[8-(1-methyl-1H-indol-6-yl)-quinoxalin-6-yl]-amine (Intermediate 24) (50.00 mg; 0.10 mmol; 1.00 eq.), Pd₂(dba)₃ (8.76 mg; 0.01 mmol; 0.10 eq.), Me₄tBuXPhos (4.60 mg; 0.01 mmol; 0.10 eq.), NaOtBu (12.87 mg; 0.13 mmol; 1.40 eq.) and ammonia solution 0.5 M in dioxane (3.25 mL; 1.63 mmol; 17.00 eq.). Purification by FCC (DCM/MeOH; gradient). Repurification by preparative HPLC. N-[(5-Aminopyridin-3-yl)methyl]-8-(1-methyl-1H-indol-6-yl)quinoxalin-6-amine (15.00 mg; yield 41%; 99% by HPLC) is obtained as a yellow powder.

### Example 76—General Procedure 31

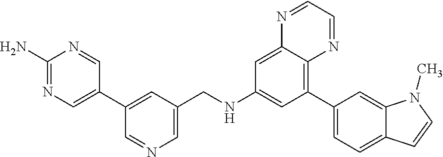 [SMILES: Cn1ccc2ccc(-c3cc(NCc4cncc(-c5cnc(N)nc5)c4)cc4nccnc34)cc21]

A sealed tube is charged with [5-[[[8-(1-methylindol-6-yl)quinoxalin-6-yl]amino]methyl]-3-pyridyl]boronic acid (Intermediate 25) (130.00 mg; 0.20 mmol; 1.00 eq.), 5-bromopyrimidin-2-ylamine (37.70 mg; 0.22 mmol; 1.10 eq.), solution of 2M Na₂CO₃ (0.30 mL; 0.59 mmol; 3.00 eq.), EtOH (2.00 mL) and toluene (2.00 mL). RM is purged with argon and then Pd(PPh₃)₄ (22.76 mg; 0.02 mmol; 0.10 eq.) is added. The resulting mixture is heated at 120° C. for 24 h. After this time, the mixture is diluted with EtOAc. The organic layer is washed with water, brine, dried over MgSO₄ and evaporated. The residue is purified by FCC (EtOAc/MeOH; gradient). N-{[5-(2-Aminopyrimidin-5-yl)pyridin-3-yl]methyl}-8-(1-methyl-1H-indol-6-yl)quinoxalin-6-amine (64.00 mg; yield 65.1%; 92% by HPLC) is obtained as a yellow powder.

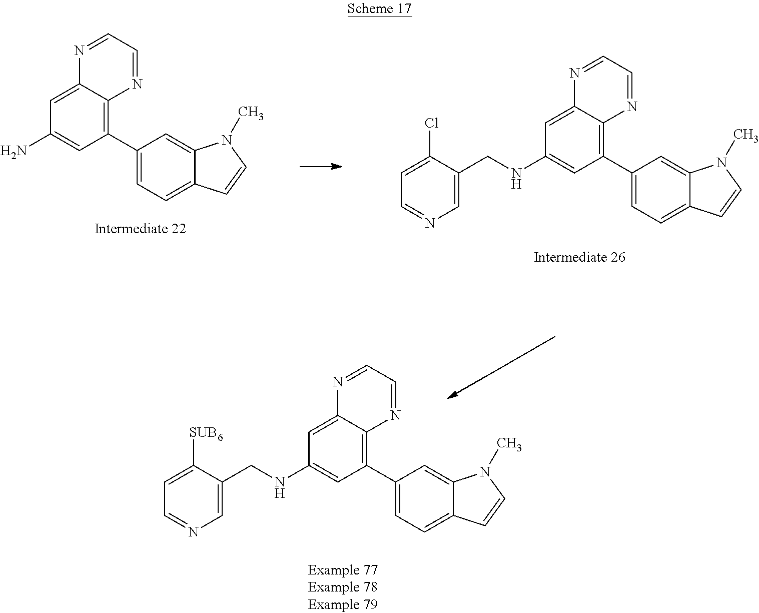

### Intermediate 26

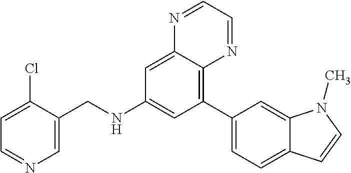 [SMILES: Cn1ccc2ccc(-c3cc(NCc4cnccc4Cl)cc4nccnc34)cc21]

The product is prepared according to General Procedure 23, described for Example 63 with 8-(1-methyl-1H-indol-6-yl)-quinoxalin-6-ylamine (Intermediate 22) (222.00 mg; 0.78 mmol; 1.00 eq.), 4-Chloro-pyridine-3-carbaldehyde (0.22 mL; 1.37 mmol; 1.75 eq.), Hantzsch ester (297.02 mg; 1.17 mmol; 1.5 eq.), TMCS (30.0 μl; 0.23 mmol; 0.30 eq.) and anhydrous DCM (5.00 mL). Purification by FCC (Hexane/EtOAc; gradient and next EtOAc/MeOH gradient). (4-Chloropyridin-3-ylmethyl)-[8-(1-methyl-1H-indol-6-yl)-quinoxalin-6-yl]-amine (160.00 mg; yield 50%; 98% by UPLC) is obtained as a bright yellow solid.

### Example 77—General Procedure 32

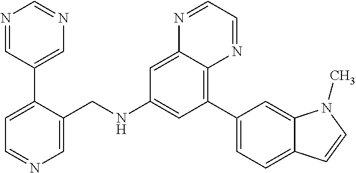 [SMILES: Cn1ccc2ccc(-c3cc(NCc4cnccc4-c4cncnc4)cc4nccnc34)cc21]

A sealed tube is charged with (4-chloropyridin-3-ylmethyl)-[8-(1-methyl-1H-indol-6-yl)quinoxalin-6-yl]-amine (40.00 mg; 0.10 mmol; 1.00 eq.) (Intermediate 26), 5-(4,4,5,5-tetramethyl-[1,3,2]dioxaborolan-2-yl)-pyrimidine (30.48 mg; 0.15 mmol; 1.50 eq.), THF (0.50 mL) and 1M solution of K₃PO₄ in water (0.30 mL; 0.30 mmol; 3.00 eq.). RM is purged with argon and then Pd(OAc)₂ (1.11 mg; 0.00 mmol; 0.05 eq.) and Xphos (4.70 mg; 0.01 mmol; 0.10 eq.) are added. RM is sealed and heated at 80° C. for 48 h. After this time, the mixture is filtered through a Celite® pad and the filtrate is diluted with EtOAc and extracted with water. Combined organic phases are washed with brine, dried over Na₂SO₄. Solvent is evaporated and the residue is purified by FCC (DCM/MeOH; gradient). Repurification by preparative HPLC. [8-(1-Methyl-1H-indol-6-yl)-quinoxalin-6-yl]-(4-pyrimidin-5-yl-pyridin-3-ylmethyl)-amine (5.00 mg; yield 11%; 97% by HPLC) is obtained as an orange powder.

### Example 78—General Procedure 33

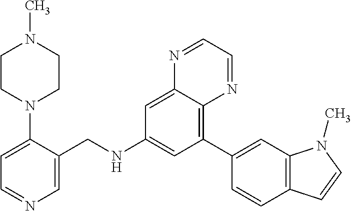 [SMILES: CN1CCN(c2ccncc2CNc2cc(-c3ccc4ccn(C)c4c3)c3nccnc3c2)CC1]

A sealed tube is charged with (4-chloropyridin-3-ylmethyl)-[8-(1-methyl-1H-indol-6-yl)-quinoxalin-6-yl]-amine (50.00 mg; 0.12 mmol; 1.00 eq.) (Intermediate 26), 1-methylpiperazine (0.06 mL; 0.5 mmol; 4.00 eq.) and Cs₂CO₃ (141.02 mg; 0.74 mmol; 6.00 eq.) and dioxane-1,4 (3.00 mL). RM is purged with argon and then Xantphos (14.26 mg; 0.02 mmol; 0.20 eq.) and Pd(OAc)₂ (5.54 mg; 0.02 mmol; 0.20 eq.) are added. RM is sealed and heated at 16h in 130° C. After this time, the mixture is filtered through a Celite® pad and the filtrate is diluted with EtOAc and extracted with water. Combined organic phases are washed with brine, dried over Na₂SO₄. Solvent is evaporated and the residue is purified by FCC (DCM/MeOH; gradient). [8-(1-Methyl-1H-indol-6-yl)-quinoxalin-6-yl]-[4-(4-methyl-piperazin-1-yl)-pyridin-3-ylmethyl]-amine (18.00 mg; yield 30.2%; 96% by HPLC) is obtained as a brown powder.

### Example 79

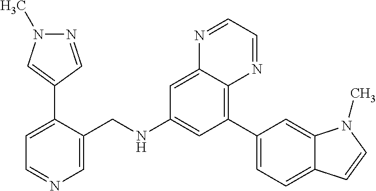 [SMILES: Cn1cc(-c2ccncc2CNc2cc(-c3ccc4ccn(C)c4c3)c3nccnc3c2)cn1]

The product is prepared according to General Procedure 26, described for Example 69 with (4-chloropyridin-3-ylmethyl)-[8-(1-methyl-1H-indol-6-yl)-quinoxalin-6-yl]-amine (Intermediate 26) (30.00 mg; 0.07 mmol; 1.00 eq.), 1-methyl-4-(4,4,5,5-tetramethyl-[1,3,2]dioxaborolan-2-yl)-1H-pyrazole (23.23 mg; 0.11 mmol; 1.50 eq.), Na₂CO₃ (0.11 mL; 0.22 mmol; 3.00 eq.), Pd(dppf)Cl2 (5.45 mg; 0.01 mmol; 0.10 eq.) and DME (2.00 mL). Purification by FCC (DCM/MeOH; gradient). [8-(1-Methyl-1H-indol-6-yl)-quinoxalin-6-yl]-[4-(1-methyl-1H-pyrazol-4-yl)-pyridin-3-ylmethyl]-amine (18.40 mg; yield 52%; 94% by HPLC) is obtained as a light yellow powder.

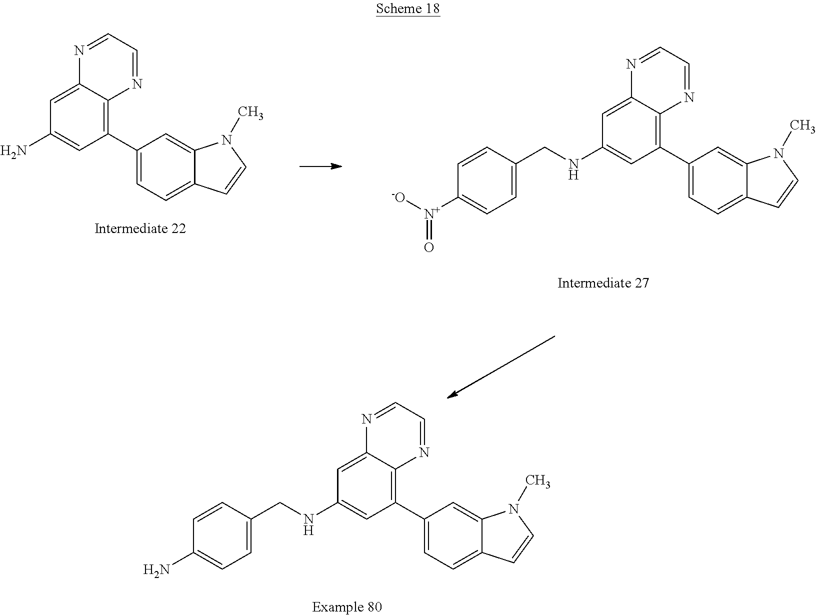

### Intermediate 27

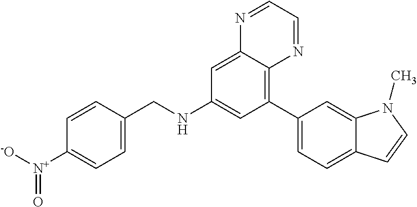

The product is prepared according to General Procedure 24, described for Example 64 with 8-(1-methyl-1H-indol-6-yl)-quinoxalin-6-ylamine (Intermediate 22) (60.00 mg; 0.22 mmol; 1.00 eq.), 4-nitrobenzaldehyde (34.71 mg; 0.23 mmol; 1.05 eq.), NaBH(OAc)₃ (58.28 mg; 0.26 mmol; 1.20 eq.), CH₃COOH (0.02 mL; 0.35 mmol; 1.60 eq.) and DCM (5.00 mL). Purification by FCC (hexane/EtOAc; gradient). 8-(1-Methyl-1H-indol-6-yl)-N-[(4-nitrophenyl)methyl]quinoxalin-6-amine (24.00 mg; yield 25%; 93% by HPLC) is obtained as a yellow powder.

### Example 80—General Procedure 34

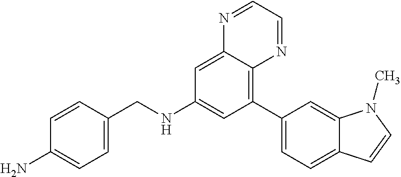 [SMILES: Cn1ccc2ccc(-c3cc(NCc4ccc(N)cc4)cc4nccnc34)cc21]

[8-(1-Methyl-1H-indol-6-yl)-quinoxalin-6-yl]-(4-nitro-benzyl)-amine (Intermediate 27) (35.00 mg; 0.08 mmol; 1.00 eq.) is dissolved in MeOH (1.00 mL). The mixture is degassed with argon and then 10% palladium on carbon (1.69 mg; 0.02 mmol; 0.20 eq.) and hydrazine monohydrate (0.08 mL; 1.59 mmol; 20.00 eq.) are added. Resulting mixture is flushed with argon again. RM is stirred at rt for 3h. The reaction is filtered through a pad of Celite® and washed with EtOAc. Organic layers are dried over Na₂SO₄ and evaporated. Product is purified by FCC (DCM/MeOH; gradient; silica gel, deactivated with NH₃). 8-(1-Methyl-1H-indol-6-yl)-N-[(3-aminophenyl)-methyl]quinoxalin-6-amine (3.10 mg; yield 8%; 84% by HPLC) is obtained as a dark film.

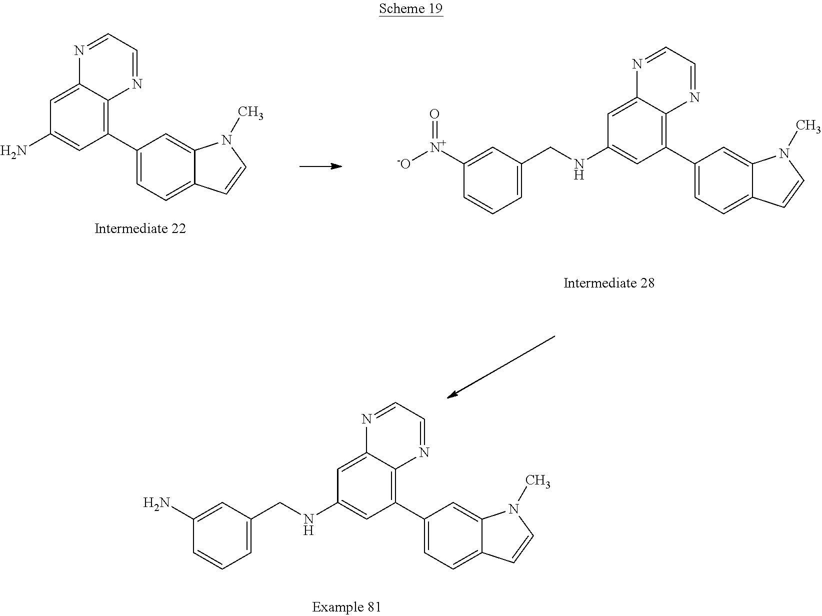

### Intermediate 28

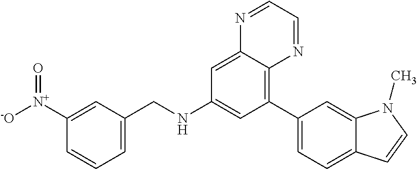

The product is prepared according to General Procedure 24, described in Example 64 with 8-(1-methyl-1H-indol-6-yl)-quinoxalin-6-ylamine (Intermediate 22) (50.00 mg; 0.18 mmol; 1.00 eq.), 3-nitrobenzaldehyde (30.30 mg; 0.20 mmol; 1.10 eq.), NaBH(OAc)₃ (48.56 mg; 0.22 mmol; 1.20 eq.), CH₃COOH (16.69 μl; 0.29 mmol; 1.60 eq.) and DCM (5.00 mL). Purification by FCC (hexane/EtOAc; gradient). 8-(1-Methyl-1H-indol-6-yl)-N-[(3-nitrophenyl)methyl]quinoxalin-6-amine (35.00 mg; yield 44%; 93% by HPLC) is obtained as a light orange solid.

### Example 81

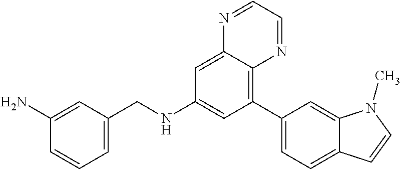 [SMILES: Cn1ccc2ccc(-c3cc(NCc4cccc(N)c4)cc4nccnc34)cc21]

The product is prepared according to General Procedure 34, described for Example 80 with [8-(1-methyl-1H-indol-6-yl)-quinoxalin-6-yl]-(3-nitrobenzyl)-amine (Intermediate 28) (60.00 mg; 0.15 mmol; 1.00 eq.), palladium on carbon 10% dry (3.12 mg; 0.03 mmol; 0.20 eq.), hydrazine monohydrate (145.07 μl; 2.93 mmol; 20.00 eq.) and MeOH (2.00 mL). Purification by FCC (DCM/MeOH; gradient; silica gel, deactivated with NH₃). N-[(3-Aminophenyl)methyl]-8-(1-methyl-1H-indol-6-yl)quinoxalin-6-amine (50.00 mg; yield 89%; 99% by HPLC) is obtained as a light orange powder.

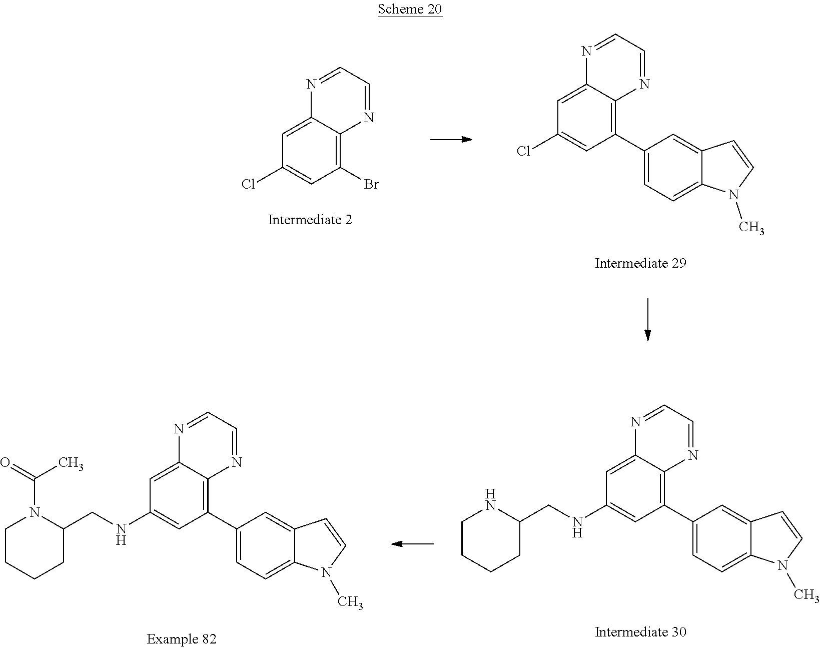 [SMILES: Clc1cc(Br)c2nccnc2c1 ; Cn1ccc2cc(-c3cc(Cl)cc4nccnc34)ccc21 ; CC(=O)N1CCCCC1CNc1cc(-c2ccc3c(ccn3C)c2)c2nccnc2c1 ; Cn1ccc2cc(-c3cc(NCC4CCCCN4)cc4nccnc34)ccc21]

### Intermediate 29

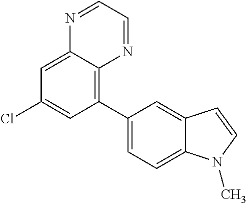 [SMILES: Cn1ccc2cc(-c3cc(Cl)cc4nccnc34)ccc21]

The product is prepared according to General Procedure 1, described for Intermediate 4 with 5-bromo-7-chloroquinoxaline (Intermediate 2) (4.0 g; 16.4 mmol; 1.0 eq.), 1-methyl-5-(4,4,5,5-tetramethyl-[1,3,2]dioxaborolan-2-yl)-1H-indole (4.35 g; 16.43 mmol; 1.00 eq.), DIPEA (5.72 mL; 32.86 mmol; 2.00 eq.), 1,4-dioxane (18 mL) and water (18 mL). Purification by FCC (hexane/EtOAc; gradient). 7-Chloro-5-(1-methyl-1H-indol-5-yl)-quinoxaline (2.70 g; yield 55%; 97% by UPLC) is obtained as a yellow solid.

### Intermediate 30

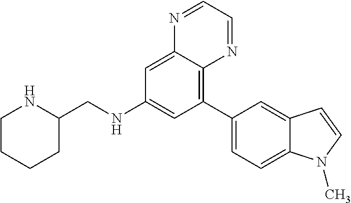 [SMILES: Cn1ccc2cc(-c3cc(NCC4CCCCN4)cc4nccnc34)ccc21]

The product is prepared according to General Procedure 5, described for Example 30 with 7-chloro-5-(1-methyl-1H-indol-5-yl)-quinoxaline (Intermediate 29) (100.00 mg; 0.34 mmol; 1.00 eq.), C-piperidin-2-yl-methylamine (99.25 μl; 0.82 mmol; 2.40 eq.), NaOtBu (45.80 mg; 0.48 mmol; 1.40 eq.), BippyPhos (13.80 mg; 0.03 mmol; 0.08 eq.), [(Cinnamyl)PdCl]₂ (8.82 mg; 0.02 mmol; 0.05 eq.) and toluene (5.00 mL). Purification by FCC (DCM/MeOH; gradient). [8-(1-Methyl-1H-indol-5-yl)-quinoxalin-6-yl]-piperidin-2-ylmethyl-amine (15.0 mg; yield 12%; 95% by UPLC) is obtained as a yellow solid.

### Example 82—General Procedure 35

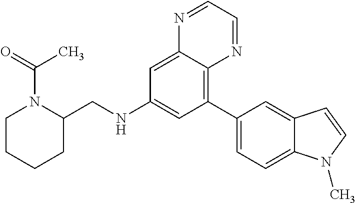 [SMILES: CC(=O)N1CCCCC1CNc1cc(-c2ccc3c(ccn3C)c2)c2nccnc2c1]

A [8-(1-methyl-1H-indol-5-yl)-quinoxalin-6-yl]-piperidin-2-ylmethyl-amine (Intermediate 30) (15.00 mg; 0.04 mmol; 1.00 eq.) is added to mixture of anhydrous DCM (10.00 mL) and TEA (144.10 μl; 1.11 mmol; 1.10 eq.). Acetyl chloride (8.90 μl; 0.13 mmol; 3.10 eq.) is added portionwise and resulted mixture is stirred at rt overnight. The reaction is quenched with water, extracted with DCM. Organic layer is washed with brine, dried over Na₂SO₄, filtered and concentrated in vacuo. The residue is purified by FCC (DCM/MeOH: gradient). 1-(2-{[8-(1-Methyl-1H-indol-5-yl)-quinoxalin-6-ylamino]-methyl}-piperidin-1-yl)-ethanone (10.00 mg; yield 55.5%; 92% by HPLC) is obtained as a yellow powder.

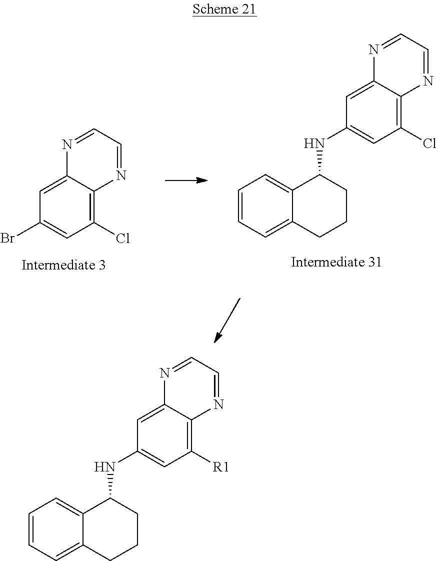

### Intermediate 31

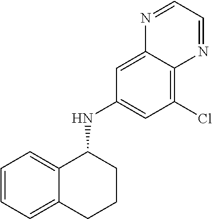 [SMILES: Clc1cc(N[C@@H]2CCCc3ccccc32)cc2nccnc12]

The product is prepared according to General Procedure 2, described in Example 1 with 7-bromo-5-chloroquinoxaline (Intermediate 3) (0.4 g; 1.64 mmol; 1.00 eq.), (R)-(1,2,3,4-tetrahydronaphthalen-1-yl)amine (0.28 mL; 1.97 mmol; 1.20 eq.), NaOtBu (0.19 g; 1.97 mmol; 1.20 eq.), BINAP (0.020 g; 0.03 mmol; 0.020 eq.), Pd₂(dba)₃ (0.015 g; 0.02 mmol; 0.010 eq.) and toluene (1.50 mL). Reaction is carried out in a MW reactor at 120° C. for 20 min. Purification by FCC (hexane/EtOAc; gradient). 8-Chloro-N-[(1R)-1,2,3,4-tetrahydronaphthalen-1-yl]quinoxalin-6-amine (470 mg; yield 92.4%; 100% by HPLC) as a yellow powder.

### Example 83

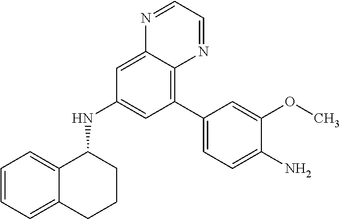 [SMILES: COc1cc(-c2cc(N[C@@H]3CCCc4ccccc43)cc3nccnc23)ccc1N]

The product is prepared according to General Procedure 26, described in Example 69 with (8-chloroquinoxalin-6-yl)-(R)-1,2,3,4-tetrahydronaphthalen-1-yl-amine (Intermediate 31) (0.090 g; 0.29 mmol; 1.00 eq.), 2-methoxy-4-(4,4,5,5-tetramethyl-[1,3,2]dioxaborolan-2-yl)-phenylamine (0.08 g; 0.32 mmol; 1.10 eq.), 1M Na₂CO₃ (1.50 mL; 1.50 mmol; 5.0 eq.), Pd(dppf)Cl₂*DCM (0.023 g; 0.03 mmol; 0.10 eq.) and DME (1.50 mL). Reaction is stirred at 110° C. overnight. Purification by FCC (DCM/MeOH; gradient). Repurification by HPLC. 8-(4-Amino-3-methoxyphenyl)-N-[(1R)-1,2,3,4-tetrahydronaphthalen-1-yl]quinoxalin-6-amine (0.083 g; yield 73%; 100% by HPLC) as a dark orange solid.

### Example 84

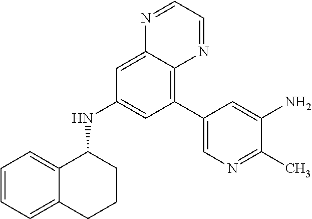 [SMILES: Cc1ncc(-c2cc(N[C@@H]3CCCc4ccccc43)cc3nccnc23)cc1N]

The product is prepared according to General Procedure 31, described in Example 76 with (8-chloroquinoxalin-6-yl)-(R)-1,2,3,4-tetrahydronaphthalen-1-yl-amine (Intermediate 31) (0.050 g; 0.16 mmol; 1.00 eq.), (5-amino-6-methyl-3-pyridyl)boronic acid (0.06 g; 0.19 mmol; 1.20 eq.), 2M Na₂CO₃ (0.16 mL; 0.32 mmol; 2.00 eq.), Pd(PPh₃)₄ (0.019 g; 0.02 mmol; 0.10 eq.) and 1,4-dioxane (2.00 mL). The mixture is stirred at 100° C. overnight. Purification by (DCM/MeOH; gradient). 8-(5-Amino-6-methylpyridin-3-yl)-N—[(1R)-1,2,3,4-tetrahydronaphthalen-1-yl]quinoxalin-6-amine (0.026 g; yield 42%; 99% by HPLC) is obtained as a yellow solid.

### Example 85

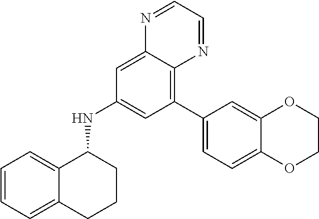 [SMILES: c1ccc2c(c1)CCC[C@H]2Nc1cc(-c2ccc3c(c2)OCCO3)c2nccnc2c1]

The product is prepared according to General Procedure 26, described in Example 69 with 8-chloroquinoxalin-6-yl)-(R)-1,2,3,4-tetrahydronaphthalen-1-yl-amine (Intermediate 31s) (80.00 mg; 0.26 mmol; 1.00 eq.), 6-(4,4,5,5-tetramethyl-[1,3,2]dioxaborolan-2-yl)-2,3-dihydrobenzo[1,4]dioxine (74.45 mg; 0.28 mmol; 1.10 eq.), Pd(dppf)Cl₂*DCM (21.09 mg; 0.03 mmol; 0.10 eq.), 1 M Na₂CO₃ (0.52 mL; 1.03 mmol; 4.00 eq.) and DME (1.00 mL). RM is stirred at 110° C. overnight. Purification by FCC (hexane/EtOAc; gradient). 8-(2,3-Dihydro-1,4-benzodioxin-6-yl)-N-[(1R)-1,2,3,4-tetrahydronaphthalen-1-yl]quinoxalin-6-amine (90.00 mg; yield 80%; 94% by HPLC) as a yellow powder.

### Example 86

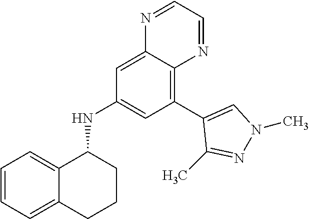 [SMILES: Cc1nn(C)cc1-c1cc(N[C@@H]2CCCc3ccccc32)cc2nccnc12]

The product is prepared according to General Procedure 28, described in Example 69 with (8-chloroquinoxalin-6-yl)-(R)-1,2,3,4-tetrahydronaphthalen-1-yl-amine (Intermediate 31) (70.00 mg; 0.23 mmol; 1.00 eq.), (1,3-dimethylpyrazol-4-yl)boronic acid (37.95 mg; 0.27 mmol; 1.20 eq.), 1 M Na₂CO₃ (1.50 mL; 1.50 mmol; 6.64 eq.), Pd(dppf)Cl₂*DCM (18.45 mg; 0.02 mmol; 0.10 eq.) and DME (1.50 mL). RM is stirred at 110° C. overnight. Purification by FCC (DCM/MeOH; gradient). Repurification by preparative HPLC. 8-(1,3-dimethyl-1H-pyrazol-4-yl)-N-[(1R)-1,2,3,4-tetrahydronaphthalen-1-yl]quinoxalin-6-amine (11.00 mg; yield 13%; 97% by HPLC) is obtained as a red solid.

### Intermediate 32

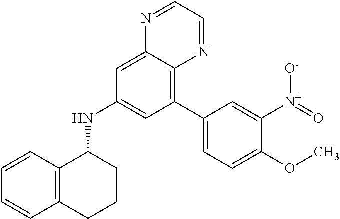 [SMILES: COc1ccc(-c2cc(N[C@@H]3CCCc4ccccc43)cc3nccnc23)cc1[N+](=O)[O-]]

The product is prepared according to General Procedure 22, described in Example 60 with (8-chloroquinoxalin-6-yl)-(R)-1,2,3,4-tetrahydronaphthalen-1-yl-amine (Intermediate 20) (80.00 mg; 0.26 mmol; 1.00 eq.), 2-(4-methoxy-3-nitro-phenyl)-4,4,5,5-tetramethyl-[1,3,2]dioxaborolane (216.22 mg; 0.77 mmol; 3.00 eq.), K₂CO₃ (107.07 mg; 0.77 mmol; 3.00 eq.), Pd(PPh₃)₄ (14.30 mg; 0.01 mmol; 0.05 eq.), water (1 mL) and 1,4-dioxane (2 mL). Purification by FCC (hexane/EtOAc; gradient). 8-(4-Methoxy-3-nitrophenyl)-N-[(1R)-1,2,3,4-tetrahydronaphthalen-1-yl]quinoxalin-6-amine (48.00 mg; yield 41%; 95% by HPLC) is obtained as a yellow amorphous solid.

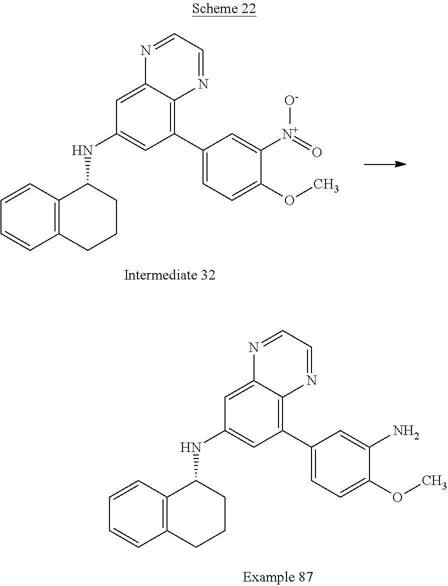 [SMILES: COc1ccc(-c2cc(N[C@@H]3CCCc4ccccc43)cc3nccnc23)cc1[N+](=O)[O-] ; COc1ccc(-c2cc(N[C@@H]3CCCc4ccccc43)cc3nccnc23)cc1N]

### Example 87—General Procedure 36

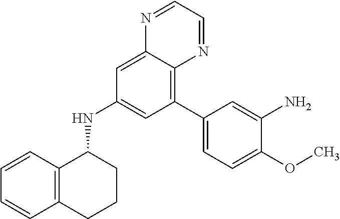 [SMILES: COc1ccc(-c2cc(N[C@@H]3CCCc4ccccc43)cc3nccnc23)cc1N]

To a suspension of Raney Ni (small spatula) in 5 mL of EtOH (9.91 mL), hydrazine monohydrate (32.28 μl; 0.43 mmol; 5.00 eq.) is added dropwise and to this mixture a suspension of 8-(4-methoxy-3-nitrophenyl)-N-[(1R)-1,2,3,4-tetrahydronaphthalen-1-yl]quinoxalin-6-amine (Intermediate 32) (38.00 mg; 0.09 mmol; 1.00 eq.) in 5 mL of EtOH is added. RM is stirred at rt for 1h. After this time, solvent is evaporated and residue is purified by FCC (hexane/EtOAc; gradient). 8-(3-amino-4-methoxyphenyl)-N-[(1R)-1,2,3,4-tetrahydronaphthalen-1-yl]quinoxalin-6-amine (8.00 mg; yield 23%; 98% by HPLC) is obtained as a yellow solid.

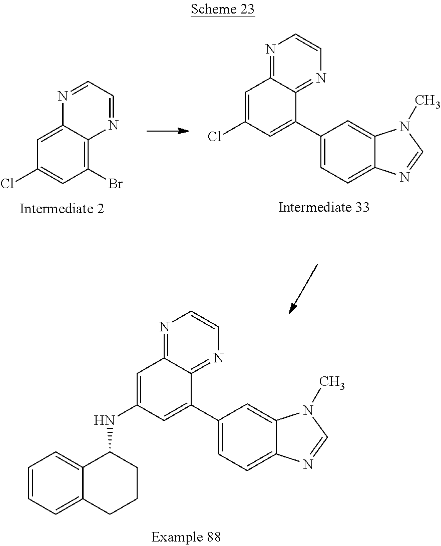 [SMILES: Clc1cc(Br)c2nccnc2c1 ; Cn1cnc2ccc(-c3cc(Cl)cc4nccnc34)cc21 ; Cn1cnc2ccc(-c3cc(N[C@@H]4CCCc5ccccc54)cc4nccnc34)cc21]

### Intermediate 33

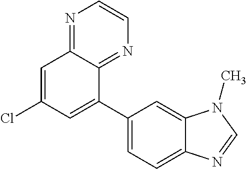 [SMILES: Cn1cnc2ccc(-c3cc(Cl)cc4nccnc34)cc21]

The product is prepared according to General Procedure 26, described in Example 69 with 5-bromo-7-chloroquinoxaline (Intermediate 2) (0.1 g; 0.41 mmol; 1.00 eq.), (3-methylbenzimidazol-5-yl)boronic acid (0.072 g; 0.41 mmol; 1.00 eq.), 1M Na₂CO₃ (1.50 mL; 1.50 mmol; 3.65 eq.), Pd(dppf)Cl₂*DCM (33.54 mg; 0.04 mmol; 0.10 eq.) and DME (1.50 mL). Reaction is stirred at 110° C. for 3h. Purification by FCC (DCM/MeOH; gradient). 7-Chloro-5-(3-methyl-3H-benzoimidazol-5-yl)-quinoxaline (0.080 g; yield 66%; 100% by UPLC) is obtained as a dark beige powder.

### Example 88

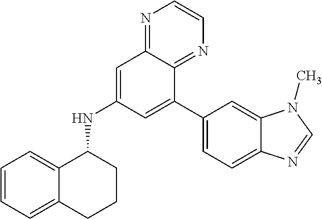 [SMILES: Cn1cnc2ccc(-c3cc(N[C@@H]4CCCc5ccccc54)cc4nccnc34)cc21]

The product is prepared according to General Procedure 2, described in Example 1 with 7-chloro-5-(3-methyl-3H-benzoimidazol-5-yl)-quinoxaline (Intermediate 33) (0.06 g; 0.20 mmol; 1.00 eq.), (R)-(1,2,3,4-tetrahydronaphthalen-1-yl)amine (0.06 mL; 0.41 mmol; 2.00 eq.), NaOtBu (0.080 g; 0.84 mmol; 3.00 eq.), BINAP (0.035 g; 0.06 mmol; 0.20 eq.), Pd₂(dba)₃ (0.012 g; 0.01 mmol; 0.10 eq.) and toluene (1.50 mL). Reaction is carried out in a MW reactor at 140° C. for 30 min. Purification by preparative HPLC. 8-(1-Methyl-1H-1,3-benzodiazol-6-yl)-N-[(1R)-1,2,3,4-tetrahydronaphthalen-1-yl]quinoxalin-6-amine (0.003 g; yield 4.0%; 97% by HPLC) is obtained as a yellow orange solid.

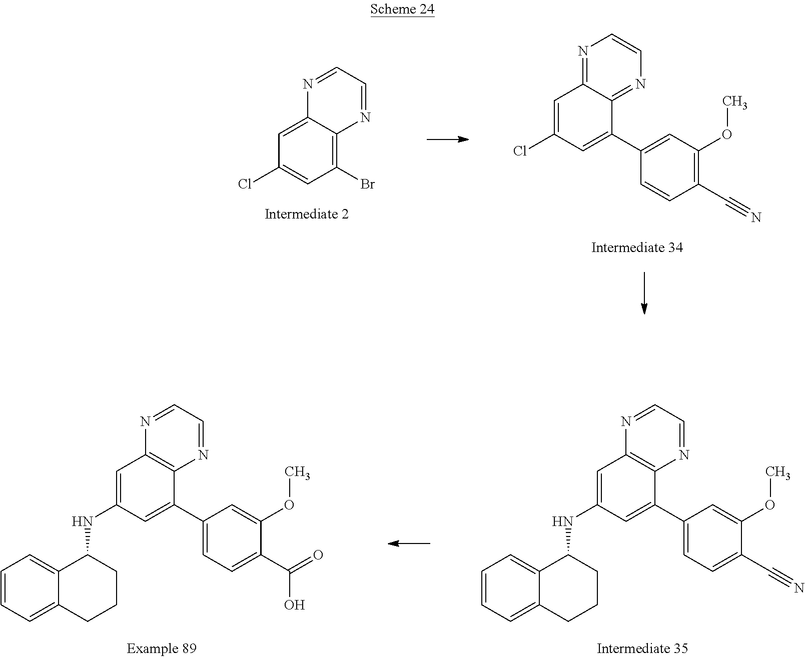 [SMILES: Clc1cc(Br)c2nccnc2c1 ; COc1cc(-c2cc(Cl)cc3nccnc23)ccc1C#N ; COc1cc(-c2cc(N[C@@H]3CCCc4ccccc43)cc3nccnc23)ccc1C(=O)O ; COc1cc(-c2cc(N[C@@H]3CCCc4ccccc43)cc3nccnc23)ccc1C#N]

### Intermediate 34

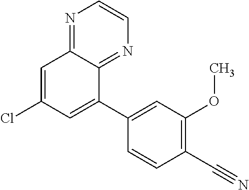 [SMILES: COc1cc(-c2cc(Cl)cc3nccnc23)ccc1C#N]

The product is prepared according to General Procedure 28, described in Example 71 with with 5-bromo-7-chloroquinoxaline (Intermediate 2) (0.15 g; 0.62 mmol; 1.00 eq.), 2-methoxy-4-(4,4,5,5-tetramethyl-[1,3,2]dioxaborolan-2-yl)-benzonitrile (0.18 g; 0.68 mmol; 1.10 eq.), K₂CO₃ (0.17 g; 1.23 mmol; 2.00 eq.), Pd(PPh₃)₄ (0.05 g; 0.04 mmol; 0.07 eq.), 1,4-dioxane (3.00 mL) and water (1.00 mL). Reaction is carried out in a MW reactor at 120° C. for 90 min. Purification by FCC (hexane/EtOAc; gradient). 4-(7-chloroquinoxalin-5-yl)-2-methoxybenzonitrile (0.096. g; yield 67%; 94% by UPLC) is obtained as a white powder. ¹H NMR (400 MHz, DMSO) δ 9.05 (d, J=1.7 Hz, 1H), 9.00 (d, J=1.7 Hz, 1H), 8.30 (d, J=2.4 Hz, 1H), 8.08 (d, J=2.4 Hz, 1H), 7.85 (d, J=7.9 Hz, 1H), 7.54 (d, J=1.0 Hz, 1H), 7.41 (dd, J=8.0, 1.3 Hz, 1H), 3.99 (s, 3H).

### Intermediate 35

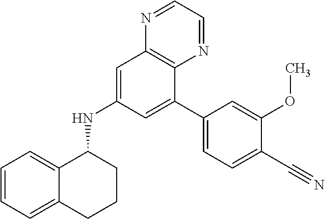 [SMILES: COc1cc(-c2cc(N[C@@H]3CCCc4ccccc43)cc3nccnc23)ccc1C#N]

The product is prepared according to General Procedure 2, described in Example 1 with 4-(7-chloroquinoxalin-5-yl)-2-methoxy-benzonitrile (Intermediate 34) (94.00 mg; 0.30 mmol; 1.00 eq.), (R)-(1,2,3,4-tetrahydronaphthalen-1-yl)amine (0.10 mL; 0.72 mmol; 2.40 eq.), NaOtBu (86.14 mg; 0.90 mmol; 3.00 eq.), BINAP (37.21 mg; 0.06 mmol; 0.20 eq.), Pd₂(dba)₃ (29.24 mg; 0.03 mmol; 0.10 eq.) and toluene (4.00 mL). Purification by FCC (hexane/EtOAc; gradient). Repurification by preparative HPLC. 2-Methoxy-4-(7-{[(1R)-1,2,3,4-tetrahydronaphthalen-1-yl]amino}quinoxalin-5-yl)benzonitrile (0.040 g; yield 27%; 99% by HPLC) is obtained as an orange powder.

¹H NMR (400 MHz, DMSO) δ 8.68 (d, J=1.9 Hz, 1H), 8.48 (d, J=1.9 Hz, 1H), 7.81 (d, J=7.9 Hz, 1H), 7.48 (d, J=2.5 Hz, 1H), 7.42 (s, 1H), 7.32 (dt, J=8.1, 3.9 Hz, 2H), 7.19 (dd, J=6.2, 3.1 Hz, 2H), 7.08 (s, 1H), 7.07 (d, J=2.5 Hz, 1H), 4.89 (s, 1H), 3.94 (s, 3H), 2.88-2.72 (m, 2H), 2.09-1.72 (m, 5H).

### Example 89—General Procedure 37

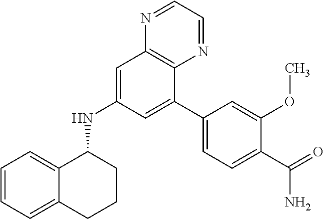 [SMILES: COc1cc(-c2cc(N[C@@H]3CCCc4ccccc43)cc3nccnc23)ccc1C(N)=O]

To a suspension of 2-methoxy-4-{7-[(R)-(1,2,3,4-tetrahydronaphthalen-1-yl)amino]-quinoxalin-5-yl}-benzonitrile (Intermediate 35) (55.00 mg; 0.13 mmol; 1.00 eq.) and K₂CO₃ (111.08 mg; 0.80 mmol; 6.00 eq.) in mixture of MeOH (2.50 mL) and water (0.50 mL), 30% solution of H₂O₂ (0.31 mL; 3.08 mmol; 23.00 eq.) is added slowly at 0° C. RM is stirred at 0° C. for 3h and then overnight at rt. The solvents are evaporated under reduced pressure and the residue is purified by FCC (hexane/EtOAc; gradient) to afford: 2-methoxy-4-(7-{[(1R)-1,2,3,4-tetrahydronaphthalen-1-yl]amino}-quinoxalin-5-yl)benzamide (12.40 mg; yield 20%; 92% by HPLC) as a yellow powder.

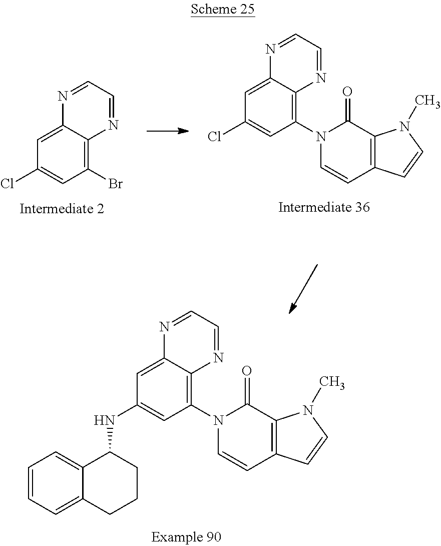 [SMILES: Clc1cc(Br)c2nccnc2c1 ; Cn1ccc2ccn(-c3cc(Cl)cc4nccnc34)c(=O)c21 ; Cn1ccc2ccn(-c3cc(N[C@@H]4CCCc5ccccc54)cc4nccnc34)c(=O)c21]

### Intermediate 36

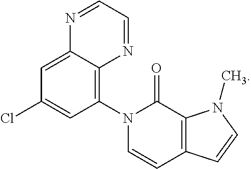 [SMILES: Cn1ccc2ccn(-c3cc(Cl)cc4nccnc34)c(=O)c21]

The product is prepared according to General Procedure 27, described in Example 70 with with 5-bromo-7-chloroquinoxaline (Intermediate 2) (50.00 mg; 0.21 mmol; 1.00 eq.), 1-methyl-1,6-dihydro-pyrrolo[2,3-c]pyridin-7-one (60.85 mg; 0.41 mmol; 2.00 eq.), K₃PO₄ (87.18 mg; 0.41 mmol; 2.00 eq.), N,N′-dimethylethylenediamine (17.68 μl; 0.16 mmol; 0.80 eq.), CuI (15.64 mg; 0.08 mmol; 0.40 eq.) and 1,4-dioxane (1.00 mL). RM is stirred at 60° C. for 5 h. Purification by FCC (hexane/EtOAc; gradient). 6-(7-Chloroquinoxalin-5-yl)-1-methyl-1,6-dihydro-pyrrolo[2,3-c]pyridin-7-one (19.00 mg; yield 20%; 67% by UPLC) is obtained as a yellow powder.

### Example 90

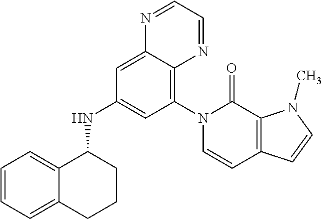 [SMILES: Cn1ccc2ccn(-c3cc(N[C@@H]4CCCc5ccccc54)cc4nccnc34)c(=O)c21]

The product is prepared according to General Procedure 2, described in Example 1 with 6-(7-chloroquinoxalin-5-yl)-1-methyl-1,6-dihydro-pyrrolo[2,3-c]pyridin-7-one (Intermediate 36) (19.00 mg; 0.04 mmol; 1.00 eq.), (R)-(1,2,3,4-tetrahydronaphthalen-1-yl)amine (11.94 μl; 0.08 mmol; 2.00 eq.), NaOtBu (11.74 mg; 0.12 mmol; 3.00 eq.), BINAP (10.14 mg; 0.02 mmol; 0.40 eq.), Pd₂(dba)₃ (7.46 mg; 0.01 mmol; 0.20 eq.) and toluene (1.00 mL). Purification by FCC (hexane/EtOAc; gradient, next EtOAc/MeOH; gradient). 1-Methyl-6-(7-{[(1R)-1,2,3,4-tetrahydronaphthalen-1-yl]amino}quinoxalin-5-yl)-1H,6H,7H-pyrrolo[2,3-c]pyridin-7-one (8.00 mg; yield 43%; 91% by HPLC) is obtained as a yellow powder.

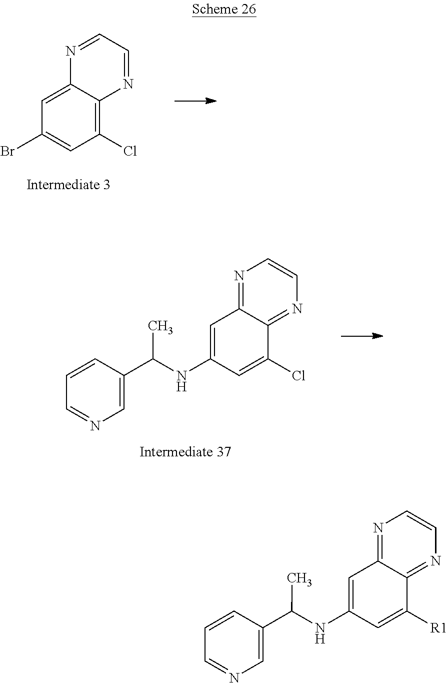

### Intermediate 37

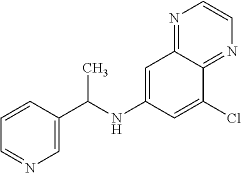 [SMILES: CC(Nc1cc(Cl)c2nccnc2c1)c1cccnc1]

The product is prepared according to General Procedure 2, described in Example 1 with 7-bromo-5-chloroquinoxaline (Intermediate 3) (300.00 mg; 1.23 mmol; 1.00 eq.), 1-pyridin-3-yl-ethylamine (0.18 mL; 1.48 mmol; 1.20 eq.), NaOtBu (142.09 mg; 1.48 mmol; 1.20 eq.), BINAP (15.34 mg; 0.02 mmol; 0.02 eq.), Pd₂(dba)₃ (11.28 mg; 0.01 mmol; 0.01 eq.) and toluene (2.00 mL). Reaction is carried out in a MW reactor at 120° C. for 50 min. Purification by FCC (DCM/MeOH; gradient). 8-Chloro-N-[1-(pyridin-3-yl)ethyl]quinoxalin-6-amine (239.00 mg; yield 65.4%; 96% by HPLC) is obtained as a yellow powder.

¹H NMR (400 MHz, DMSO) δ 8.67 (d, J=10.8 Hz, 2H), 8.54 (s, 1H), 8.43 (d, J=6.3 Hz, 1H), 7.81 (d, J=7.9 Hz, 1H), 7.57 (s, 1H), 7.35 (d, J=12.1 Hz, 2H), 6.60 (s, 1H), 4.81 (s, 1H), 1.53 (d, J=6.8 Hz, 3H).

### Example 91—General Procedure 29

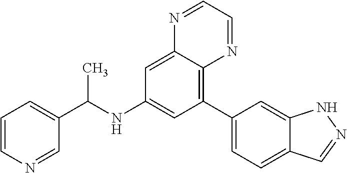 [SMILES: CC(Nc1cc(-c2ccc3cn[nH]c3c2)c2nccnc2c1)c1cccnc1]

A sealed tube is charged with (8-chloroquinoxalin-6-yl)-(1-pyridin-3-ylethyl)-amine (Intermediate 37) (90.00 mg; 0.32 mmol; 1.00 eq.), 1H-indazole-6-boronic acid pinacol ester (92.58 mg; 0.38 mmol; 1.20 eq.), K₂CO₃ (131.04 mg; 0.95 mmol; 3.00 eq.), DME (1.50 mL) and water (1.50 mL). The suspension is purged with argon and then Pd(dppf)Cl₂*DCM (25.81 mg; 0.03 mmol; 0.10 eq.) is added. RM is stirred at 85° C. for 2 h. After this time, the mixture is filtered through a Celite® pad and the filtrate is diluted with EtOAc and extracted with water. Organic phase is washed with brine, dried over Na₂SO₄ and then solvent is evaporated. Crude product is purified on FCC (DCM/MeOH; gradient) to afford 8-(1H-indazol-6-yl)-N-[1-(pyridin-3-yl)ethyl]-quinoxalin-6-amine (96.00 mg; yield 82%; 99% by HPLC) as a brown solid.

### Example 92

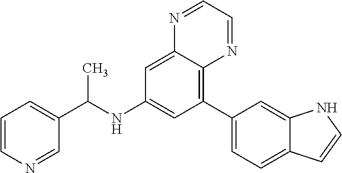 [SMILES: CC(Nc1cc(-c2ccc3cc[nH]c3c2)c2nccnc2c1)c1cccnc1]

The product is prepared according to General Procedure 31, described in Example 76 with (8-chloroquinoxalin-6-yl)-(1-pyridin-3-yl-ethyl)-amine (Intermediate 37) (60.00 mg; 0.21 mmol; 1.00 eq.), 6-(4,4,5,5-tetramethyl-[1,3,2]dioxaborolan-2-yl)-1H-indole (112.70 mg; 0.46 mmol; 2.20 eq.), 2M Na₂CO₃ (0.21 mL; 0.42 mmol; 2.00 eq.), Pd(PPh₃)₄ (48.68 mg; 0.04 mmol; 0.20 eq.) and 1,4-dioxane (1.00 mL). RM is stirred for 20 h at 100° C. Purification by FCC (DCM/MeOH; gradient). 8-(1H-Indol-6-yl)-N-[1-(pyridin-3-yl)ethyl]quinoxalin-6-amine (24.00 mg; yield 30%; 97% by HPLC) is obtained as a yellow solid.

### Example 93—General Procedure 39

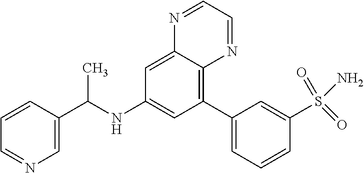 [SMILES: CC(Nc1cc(-c2cccc(S(N)(=O)=O)c2)c2nccnc2c1)c1cccnc1]

A sealed tube is charged with (8-chloroquinoxalin-6-yl)-(1-pyridin-3-yl-ethyl)-amine (Intermediate 37) (60.00 mg; 0.20 mmol; 1.00 eq.), 3-(4,4,5,5-tetramethyl-[1,3,2]dioxaborolan-2-yl)-benzenesulfonamide (112.16 mg; 0.31 mmol; 1.50 eq.), Cs₂CO₃ (199.99 mg; 0.61 mmol; 3.00 eq.), DME (2.00 mL) and water (1.00 mL). The suspension is purged with argon and then Pd(dppf)₂Cl₂*DCM (25.55 mg; 0.03 mmol; 0.15 eq.) is added. RM is sealed and heated at 120° C. for 16h. After this time, the mixture is filtered through a Celite® pad and the filtrate is diluted with EtOAc and extracted with water. Organic phase is washed with brine, dried over Na₂SO₄ and then solvent is evaporated. Crude product is purified by FCC (hexane/EtOAc; gradient) to afford 3-[7-(1-Pyridin-3-yl-ethylamino)-quinoxalin-5-yl]-benzenesulfonamide (46.00 mg; yield 55%: 99% by HPLC) as a yellow powder.

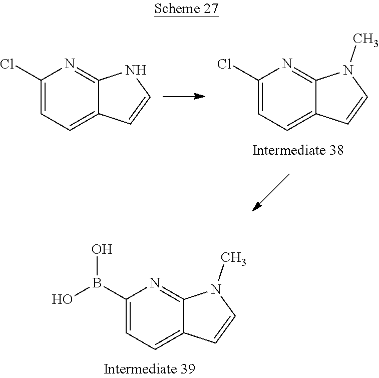

### Intermediate 38—General Procedure 40

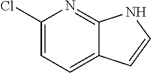

A NaH 60% in oil (100.92 mg; 2.52 mmol; 1.10 eq.) is added portionwise to a solution of 6-chloro-1H-pyrrolo[2,3-b]pyridine (350.00 mg; 2.29 mmol; 1.00 eq.) in anhydrous DMF (5.00 mL) at 0-5° C. After stirring for 30 minutes CH₃I (0.10 mL; 1.61 mmol; 0.70 eq.) is added dropwise. After addition, RM is stirred at 0-5° C. for 30 min and then at rt for 1 h. Reaction is quenched with water and extracted with EtOAc. Organic phase is washed with water, brine, dried over Na₂SO₄, filtered and concentrated to afford 6-chloro-1-methyl-1H-pyrrolo[2,3-b]pyridine (332.00 mg; yield 85%; 98% by UPLC) as a brown oil. The crude product is used in the next step.

### Intermediate 39 General Procedure 41

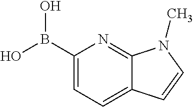

A sealed tube is charged with 6-chloro-1-methyl-1H-pyrrolo[2,3-b]pyridine (Intermediate 38) (320.00 mg; 1.88 mmol; 1.00 eq.), bis(pinacolato)diboron (573.58 mg; 2.26 mmol; 1.20 eq.), KOAc (923.65 mg; 9.41 mmol; 5.00 eq.) and 1,4-dioxane (8.00 mL). The suspension is purged with argon and then Pd(dppf)Cl₂*DCM (153.71 mg; 0.19 mmol; 0.10 eq.) is added. RM is sealed and heated at 100° C. for 18 h. After this time, the mixture is filtered through a Celite® pad and the filtrate is diluted with n-BuOH and extracted with water. Organic phase is washed with brine, dried over Na₂SO₄ and then solvent is evaporated. Crude (1-methylpyrrolo[2,3-b]pyridin-6-yl)boronic acid (2.00 g; yield 259%) as a brown solid is used for further reactions.

### Example 94

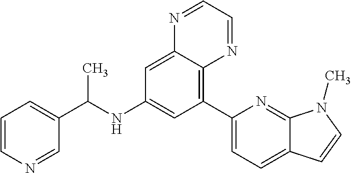 [SMILES: CC(Nc1cc(-c2ccc3ccn(C)c3n2)c2nccnc2c1)c1cccnc1]

The product is prepared according to General Procedure 31, described in Example 76 with (8-chloroquinoxalin-6-yl)-(1-pyridin-3-ylethyl)-amine (Intermediate 37) (70.00 mg; 0.25 mmol; 1.00 eq.), (1-methylpyrrolo[2,3-b]pyridin-6-yl)boronic acid (540.78 mg; 1.22 mmol; 5.00 eq.) (Intermediate 39), 2M Na₂CO₃ (0.25 mL; 0.49 mmol; 2.00 eq.), Pd(PPh₃)₄ (56.78 mg; 0.04 mmol; 0.20 eq.) and 1,4-dioxane (2.00 mL). RM is stirred at 100° C. for 8h. Purification by FCC (DCM/MeOH; gradient). Repurification by preparative HPLC and after evaporation extraction with saturated NaHCO₃ solution is done. 8-{1-Methyl-1H-pyrrolo[2,3-b]pyridin-6-yl}-N-[1-(pyridin-3-yl)ethyl]quinoxalin-6-amine (6.80 mg; yield 7%; 100% by HPLC) is obtained as a yellow solid.

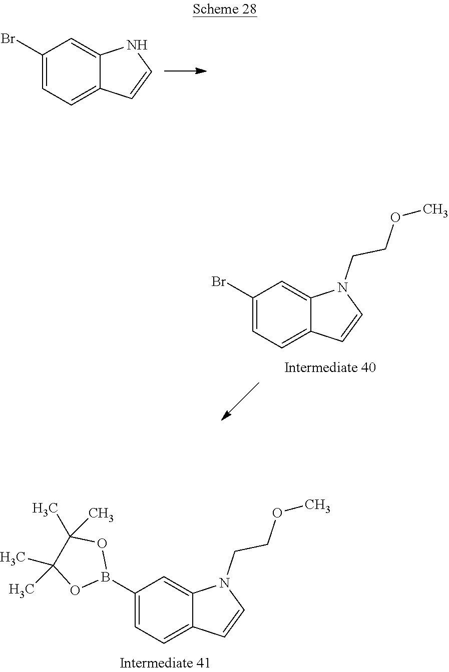

### Intermediate 40

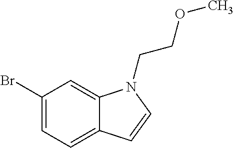

The product is prepared according to General Procedure 40, described for Intermediate 38 with 6-bromo-1H-indole (1.00 g; 5.10 mmol; 1.00 eq.), NaH 60% in oil (0.24 g; 6.12 mmol; 1.20 eq.), 2-bromoethyl methyl ether (0.58 mL; 6.12 mmol; 1.20 eq.) and anhydrous DMF (15.00 mL). RM is stirred at rt overnight. Crude 6-bromo-1-(2-methoxyethyl)-1H-indole (1.27 g; yield 93%; 95% by UPLC) as a dark red oil is used for further reactions.

### Intermediate 41

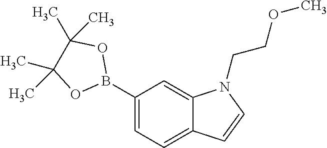

The product is prepared according to General Procedure 41, described for Intermediate 39 with 6-bromo-1-(2-methoxyethyl)-1H-indole (Intermediate 40) (1.27 g; 4.25 mmol; 1.00 eq.), bis(pinacolato)diboron (1.40 g; 5.52 mmol; 1.30 eq.), KOAc (0.83 g; 8.50 mmol; 2.00 eq.), Pd(dppf)Cl₂ (31.08 mg; 0.04 mmol; 0.01 eq.) and 1,4-dioxane (10.00 mL). Purification by FCC (hexane/EtOAc; gradient). 1-(2-Methoxyethyl)-6-(4,4,5,5-tetramethyl-[1,3,2]dioxaborolan-2-yl)-1H-indole (1.15 g; yield 89%; 99% by UPLC) is obtained as a light yellow oil.

### Example 95

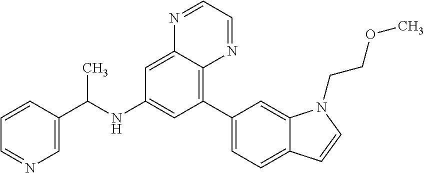 [SMILES: COCCn1ccc2ccc(-c3cc(NC(C)c4cccnc4)cc4nccnc34)cc21]

The product is prepared according to General Procedure 28 described in Example 71 with (8-chloroquinoxalin-6-yl)-(1-pyridin-3-yl-ethyl)-amine (Intermediate 37) (100.00 mg; 0.35 mmol; 1.00 eq.), 1-(2-methoxyethyl)-6-(4,4,5,5-tetramethyl-[1,3,2]dioxaborolan-2-yl)-1H-indole (Intermediate 41) (126.93 mg; 0.42 mmol; 1.20 eq.), K₂CO₃ (145.61 mg; 1.05 mmol; 3.00 eq.), Pd(PPh₃)₄ (81.12 mg; 0.08 mmol; 0.20 eq.), 1,4-dioxane (1.00 mL) and water (0.50 mL). Purification by FCC (hexane/EtOAc; gradient). 8-[1-(2-Methoxyethyl)-1H-indol-6-yl]-N-[1-(pyridin-3-yl)ethyl]quinoxalin-6-amine (108.00 mg; yield 69%; 95% by HPLC) is obtained as a green solid.

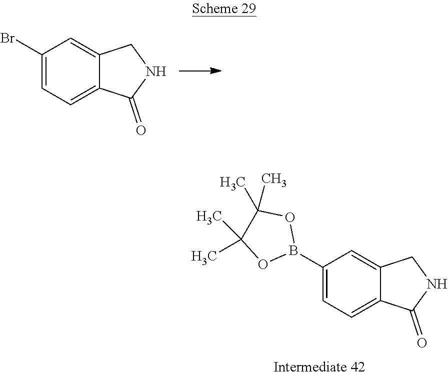 [SMILES: O=C1NCc2cc(Br)ccc21 ; CC1(C)OB(c2ccc3c(c2)CNC3=O)OC1(C)C]

### Intermediate 42—General Procedure 42

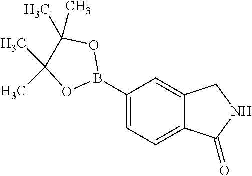 [SMILES: CC1(C)OB(c2ccc3c(c2)CNC3=O)OC1(C)C]

To a solution of 5-bromo-2,3-dihydro-isoindol-1-one (200.00 mg; 0.90 mmol; 1.00 eq.) in anhydrous 1,4-dioxane (4.00 mL), bis(pinacolato)diboron (255.40 mg; 0.99 mmol; 1.10 eq.), KOAc (307.79 mg; 3.14 mmol; 3.50 eq.) and dppf (49.68 mg; 0.09 mmol; 0.10 eq.) are added. RM is purged with argon and Pd(dppf)Cl₂ (65.54 mg; 0.09 mmol; 0.10 eq.) is added. RM is stirred at 110° C. for 16 h. RM is filtered through a pad of Celite® and partitioned between EtOAc and water. The aqueous phase is washed with EtOAc, combined organic layers are washed with brine, dried over Na₂SO₄ and concentrated in vacuo to afford 5-(4,4,5,5-tetramethyl-[1,3,2]dioxaborolan-2-yl)-2,3-dihydro-isoindol-1-one (377.00 mg; 68%; 42% by UPLC) as a brown powder. Crude product is used for further reactions.

### Example 96

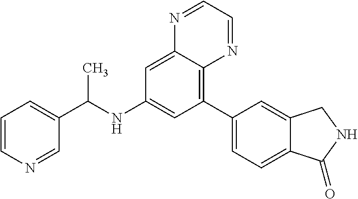 [SMILES: CC(Nc1cc(-c2ccc3c(c2)CNC3=O)c2nccnc2c1)c1cccnc1]

The product is prepared according to General Procedure 28, described in Example 71 with (8-chloroquinoxalin-6-yl)-(1-pyridin-3-yl-ethyl)-amine (Intermediate 37) (80.00 mg; 0.22 mmol; 1.00 eq.), 5-(4,4,5,5-tetramethyl-[1,3,2]dioxaborolan-2-yl)-2,3-dihydro-isoindol-1-one (Intermediate 31) (152.53 mg; 0.25 mmol; 1.10 eq.), 1 M Na₂CO₃ (0.45 mL; 0.90 mmol; 4.00 eq.), Pd(dppf)Cl₂*DCM (18.36 mg; 0.02 mmol; 0.10 eq.) and DME. Purification by FCC (hexane/EtOAC; gradient). 5-(7-{[1-(Pyridin-3-yl)ethyl]-amino}quinoxalin-5-yl)-2,3-dihydro-1H-isoindol-1-one (27.00 mg; 0.07 mmol; yield 31%; 99% by HPLC) is obtained as a yellow-brown powder.

### Example 97

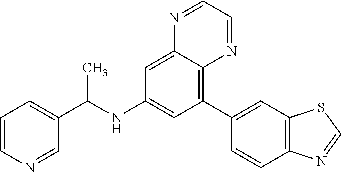 [SMILES: CC(Nc1cc(-c2ccc3ncsc3c2)c2nccnc2c1)c1cccnc1]

The product is prepared according to General Procedure 28, described in Example 71 with (8-chloroquinoxalin-6-yl)-(1-pyridin-3-yl-ethyl)-amine (Intermediate 37) (55.00 mg; 0.19 mmol; 1.00 eq.), 6-(4,4,5,5-tetramethyl-[1,3,2]dioxaborolan-2-yl)-benzothiazole (60.53 mg; 0.23 mmol; 1.20 eq.), K₂CO₃ (80.09 mg; 0.58 mmol; 3.00 eq.), Pd(PPh₃)₄ (44.62 mg; 0.04 mmol; 0.20 eq.), 1,4-dioxane (1.00 mL) and water (0.50 mL). RM is stirred at 100° C. for 24h. Purification by FCC (DCM/MeOH; gradient). 8-(1,3-benzothiazol-6-yl)-N-[1-(pyridin-3-yl)ethyl]quinoxalin-6-amine (23.00 mg; yield 28%; 91% by HPLC) is obtained as a yellow solid.

### Example 98

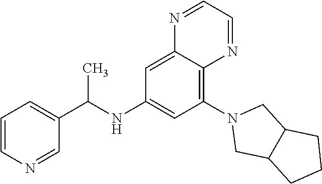 [SMILES: CC(Nc1cc(N2CC3CCCC3C2)c2nccnc2c1)c1cccnc1]

The product is prepared according to General Procedure 2, described in Example 1 with (8-chloroquinoxalin-6-yl)-(1-pyridin-3-yl-ethyl)-amine (Intermediate 37) (30.00 mg; 0.11 mmol; 1.00 eq.), octahydrocyclopenta[c]pyrrole hydrochloride (23.33 mg; 0.16 mmol; 1.50 eq.), NaOtBu (3.00 eq.), BINAP (1.31 mg; 0.00 mmol; 0.02 eq.), Pd₂(dba)₃ (4.89 mg; 0.00 mmol; 0.02 eq.) and toluene (1.00 mL). RM is stirred at 140° C. for 24 h. Purification by FCC (hexane/EtOAc; gradient and next EtOAc/MeOH; gradient). 8-{octahydrocyclopenta[c]pyrrol-2-yl}-N-[1-(pyridin-3-yl)ethyl]quinoxalin-6-amine (17.00 mg; yield 42%; 93% by HPLC) is obtained as a dark brown powder.

### Example 99

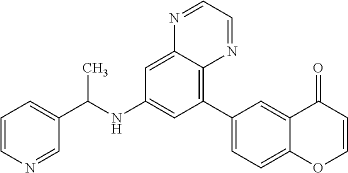 [SMILES: CC(Nc1cc(-c2ccc3occc(=O)c3c2)c2nccnc2c1)c1cccnc1]

The product is prepared according to General Procedure 38, described in Example 91 with (8-chloroquinoxalin-6-yl)-(1-pyridin-3-yl-ethyl)-amine (Intermediate 37) (100.00 mg; 0.35 mmol; 1.00 eq.), 6-(4,4,5,5-tetramethyl-[1,3,2]dioxaborolan-2-yl)-chromen-4-one (95.56 mg; 0.35 mmol; 1.00 eq.), K₂CO₃ (145.61 mg; 1.05 mmol; 3.00 eq.), Pd(dppf)Cl₂ (24.65 mg; 0.04 mmol; 0.10 eq.), DME (1 mL) and water (1 mL). RM is stirred at 100° C. overnight. Purification by FCC (hexane/EtOAc; gradient). Repurification by preparative HPLC and after evaporation extraction with saturated NaHCO₃ solution is done. 6-(7-{[1-(Pyridin-3-yl)ethyl]amino}quinoxalin-5-yl)-4H-chromen-4-one (14.00 mg; yield 10%; 94% by HPLC) is obtained as a yellow powder.

### Example 100—General Procedure 43

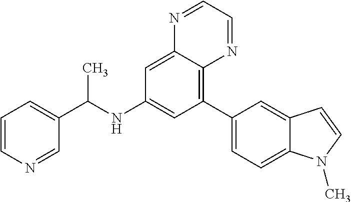 [SMILES: CC(Nc1cc(-c2ccc3c(ccn3C)c2)c2nccnc2c1)c1cccnc1]

A sealed tube is charged with (8-chloroquinoxalin-6-yl)-(1-pyridin-3-ylethyl)amine (Intermediate 37) (70.00 mg; 0.25 mmol; 1.00 eq.), 1-methylindole-5-boronic acid pinacol ester (66.37 mg; 0.26 mmol; 1.05 eq.), K₂CO₃ (101.92 mg; 0.74 mmol; 3.00 eq.), 1,4-dioxane (4.50 mL) and water (1.50 mL). The suspension is purged with argon and then Pd(dppf)Cl₂*DCM (20.08 mg; 0.02 mmol; 0.10 eq.) is added. RM is stirred at 110° C. for 16 h. After this time, the mixture is filtered through a Celite® pad and the filtrate is diluted with DCM and extracted with water. Organic phase is washed with brine, dried over Na₂SO₄ and then solvent is evaporated. Crude product is purified by FCC (DCM/MeOH; gradient). Repurification by preparative HPLC. 8-(1-Methyl-1H-indol-5-yl)-N-[1-(pyridin-3-yl)ethyl]quinoxalin-6-amine (14.00 mg; yield 15.0%; 99% by HPLC) is obtained as a yellow solid.

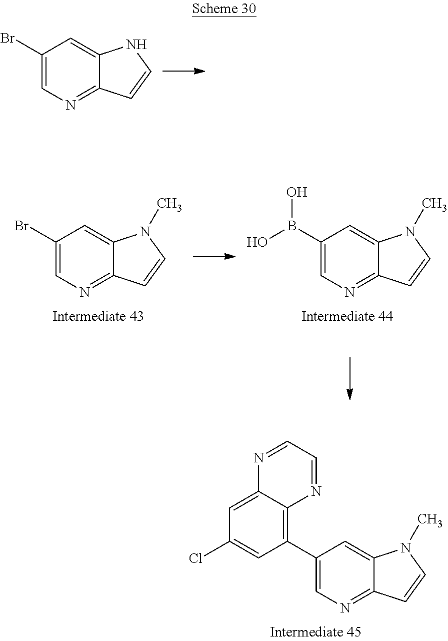 [SMILES: Brc1cnc2cc[nH]c2c1 ; Cn1ccc2ncc(Br)cc21 ; Cn1ccc2ncc(B(O)O)cc21 ; Cn1ccc2ncc(-c3cc(Cl)cc4nccnc34)cc21]

### Intermediate 43

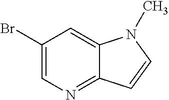

The product is prepared according to General Procedure 40, described for Intermediate 38 with NaH 60% in oil (66.99 mg; 1.67 mmol; 1.10 eq.), 6-bromo-1H-pyrrolo[3,2-b]pyridine (300.00 mg; 1.52 mmol; 1.00 eq.), CH₃I (0.11 mL; 1.83 mmol; 1.20 eq.) and anhydrous DMF (5 mL). RM is stirred at 0-5° C. for 30 min and next 1 h at rt. Crude 6-bromo-1-methyl-1H-pyrrolo[3,2-b]pyridine (330.00 mg; yield; 53.4%;) is used in the next step without purification.

### Intermediate 44

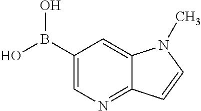

The product is prepared according to General Procedure 41, described for Intermediate 39 with 6-bromo-1-methyl-1H-pyrrolo[3,2-b]pyridine (Intermediate 43) (330.00 mg; 0.81 mmol; 1.00 eq.), bis(pinacolato)diboron (227.11 mg; 0.89 mmol; 1.10 eq.), KOAc (398.97 mg; 4.07 mmol; 5.00 eq.), Pd(dppf)Cl₂*DCM (66.40 mg; 0.08 mmol; 0.10 eq.) and 1,4-dioxane (4.00 mL). (1-methylpyrrolo[3,2-b]pyridin-6-yl)boronic acid (200.00 mg; yield 90.9%; 65% by UPLC) is used in the next step without purification.

### Intermediate 45

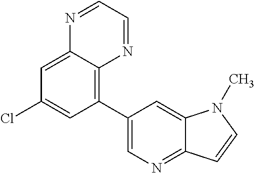 [SMILES: Cn1ccc2ncc(-c3cc(Cl)cc4nccnc34)cc21]

The product is prepared according to General Procedure 1, described for Intermediate 4 with 5-bromo-7-chloroquinoxaline (Intermediate 2) (0.07 mL; 0.49 mmol; 1.00 eq.), (1-methylpyrrolo[3,2-b]pyridin-6-yl)boronic acid (Intermediate 44) (173.46 mg; 0.59 mmol; 1.20 eq.), DIPEA (0.17 mL; 0.99 mmol; 2.00 eq.), Pd(dppf)Cl₂ (36.05 mg; 0.05 mmol; 0.10 eq.), 1,4-dioxane (4.00 mL) and water (4.00 mL). RM is heated to 85° C. for 3 h. Purification by FCC (hexane/EtOAct; gradient). 7-Chloro-5-(1-methyl-1H-pyrrolo[3,2-b]pyridin-6-yl)-quinoxaline (160.00 mg; yield 95%; 87% by UPLC) is obtained as a yellow solid.

¹H NMR (400 MHz, DMSO) δ 8.67 (d, J=10.8 Hz, 2H), 8.54 (s, 1H), 8.43 (d, J=6.3 Hz, 1H), 7.81 (d, J=7.9 Hz, 1H), 7.57 (s, 1H), 7.35 (d, J=12.1 Hz, 2H), 6.60 (s, 1H), 4.81 (s, 1H), 1.53 (d, J=6.8 Hz, 3H).

### Example 101

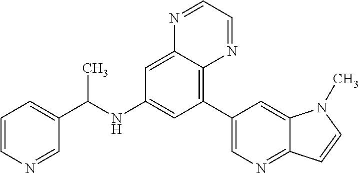 [SMILES: CC(Nc1cc(-c2cnc3ccn(C)c3c2)c2nccnc2c1)c1cccnc1]

The product is prepared according to General Procedure 2, described in Example 1 with 7-chloro-5-(1-methyl-1H-pyrrolo[3,2-b]pyridin-6-yl)-quinoxaline (Intermediate 45) (170.00 mg; 0.43 mmol; 1.00 eq.), 1-pyridin-3-yl-ethylamine (0.10 mL; 0.85 mmol; 2.00 eq.), NaOtBu (205.09 mg; 2.13 mmol; 5.00 eq.), BINAP (53.15 mg; 0.09 mmol; 0.20 eq.), Pd₂(dba)₃ (39.08 mg; 0.04 mmol; 0.10 eq.) and toluene (3.00 mL). Purification by FCC (DCM/MeOH; gradient). 8-{1-Methyl-1H-pyrrolo[3,2-b]pyridin-6-yl}-N-[1-(pyridin-3-yl)ethyl]quinoxalin-6-amine (60.00 mg; 0.15 mmol; yield 36%; 97% by HPLC) is obtained as a brown powder.

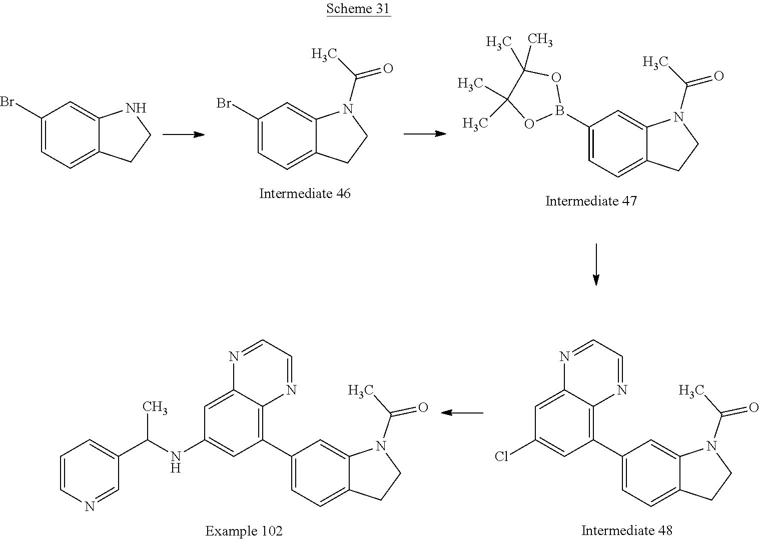 [SMILES: Brc1ccc2c(c1)NCC2 ; CC(=O)N1CCc2ccc(Br)cc21 ; CC(=O)N1CCc2ccc(B3OC(C)(C)C(C)(C)O3)cc21 ; CC(=O)N1CCc2ccc(-c3cc(NC(C)c4cccnc4)cc4nccnc34)cc21 ; CC(=O)N1CCc2ccc(-c3cc(Cl)cc4nccnc34)cc21]

### Intermediate 46

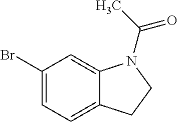

The product is prepared according to General Procedure 35, described in Example 82 with 6-bromo-2,3-dihydro-1H-indole (200.00 mg; 1.01 mmol; 1.00 eq.), acetyl chloride (78.98 μl; 1.11 mmol; 1.10 eq.), TEA (144.10 μl; 1.11 mmol; 1.10 eq.) and anhydrous THF (10.00 mL). The reaction is quenched with water, extracted with EtOAc. Organic layer is washed with brine, dried over Na₂SO₄, filtered and concentrated in vacuo to afford 1-(6-bromo-2,3-dihydroindol-1-yl) ethanone (380.00 mg; yield 99%; 64% by UPLC) as a dark gray solid.

### Intermediate 47

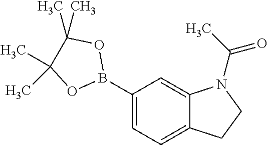 [SMILES: CC(=O)N1CCc2ccc(B3OC(C)(C)C(C)(C)O3)cc21]

The product is prepared according to General Procedure 41, described for Intermediate 39 with 1-(6-bromo-2,3-dihydroindol-1-yl) (Intermediate 46) (200.00 mg; 0.83 mmol; 1.00 eq.), bis(pinacolato)diboron (274.99 mg; 1.08 mmol; 1.30 eq.), KOAc (163.50 mg; 1.67 mmol; 2.00 eq.), Pd(dppf)Cl2 (14.63 mg; 0.02 mmol; 0.02 eq.) and 1,4-dioxane (5.00 mL). Purification by FCC (hexane/EtOAc; gradient). 1-[6-(4,4,5,5-Tetramethyl-[1,3,2]dioxaborolan-2-yl)-2,3-dihydroindol-1-yl]-ethanone (150.00 mg; yield 41%; 65% by UPLC) is obtained as a light yellow solid.

### Intermediate 48

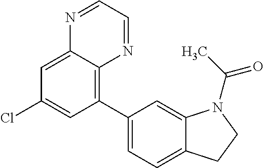 [SMILES: CC(=O)N1CCc2ccc(-c3cc(Cl)cc4nccnc34)cc21]

The product is prepared according to General Procedure 28, described in Example 71 with 5-bromo-7-chloroquinoxaline (Intermediate 2) (42.00 mg; 0.17 mmol; 1.00 eq.), 1-[6-(4,4,5,5-tetramethyl-[1,3,2]dioxaborolan-2-yl)-2,3-dihydro-indol-1-yl]-ethanone (Intermediate 47) (49.53 mg; 0.17 mmol; 1.00 eq.), K₂CO₃ (71.52 mg; 0.52 mmol; 3.00 eq.), Pd(PPh₃)₄ (1.99 mg; 0.00 mmol; 0.01 eq.), 1,4-dioxane (2 mL) and water (2 mL). Purification by FFC (hexane/EtOAc; gradient). 1-[6-(7-Chloro-quinoxalin-5-yl)-2,3-dihydro-indol-1-yl]-ethanone (31.00 mg; yield 37%, 66% by UPLC) is obtained as a light yellow solid.

### Example 102

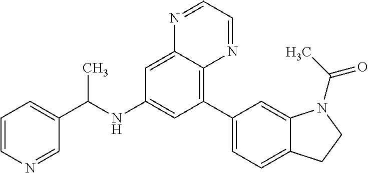 [SMILES: CC(=O)N1CCc2ccc(-c3cc(NC(C)c4cccnc4)cc4nccnc34)cc21]

The product is prepared according to General Procedure 2, described in Example 1 with 1-[6-(7-chloroquinoxalin-5-yl)-2,3-dihydro-indol-1-yl]-ethanone (Intermediate 48) (31.00 mg; 0.06 mmol; 1.00 eq.), 1-pyridin-3-yl-ethylamine (9.26 μl; 0.08 mmol; 1.20 eq.), NaOtBu (7.29 mg; 0.08 mmol; 1.20 eq.), BINAP (1.57 mg; 0.00 mmol; 0.04 eq.) and Pd₂(dba)₃ (1.16 mg; 0.00 mmol; 0.02 eq.) and toluene (1.00 mL). RM is stirred at 130° C. overnight. Purification by FCC (DCM/MeOH; gradient). 1-[6-(7-{[1-(Pyridin-3-yl)ethyl]-amino}quinoxalin-5-yl)-2,3-dihydro-1H-indol-1-yl]ethan-1-one (12.00 mg; yield 44%; 95% by HPLC) is obtained as a yellow amorphous powder.

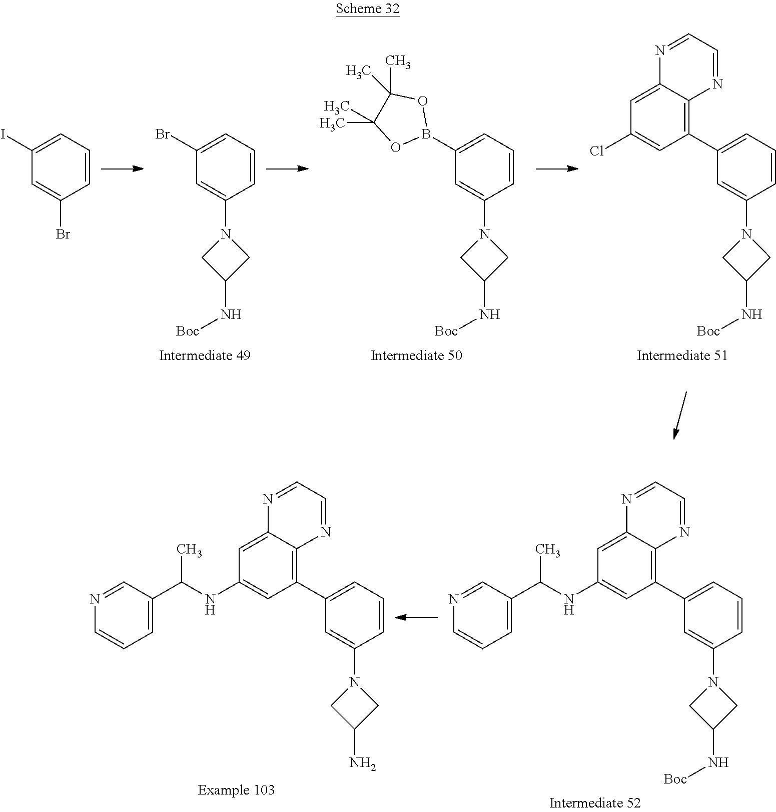 [SMILES: Brc1cccc(I)c1 ; CC(C)(C)OC(=O)NC1CN(c2cccc(Br)c2)C1 ; CC(C)(C)OC(=O)NC1CN(c2cccc(B3OC(C)(C)C(C)(C)O3)c2)C1 ; CC(C)(C)OC(=O)NC1CN(c2cccc(-c3cc(Cl)cc4nccnc34)c2)C1 ; CC(Nc1cc(-c2cccc(N3CC(N)C3)c2)c2nccnc2c1)c1cccnc1 ; CC(Nc1cc(-c2cccc(N3CC(NC(=O)OC(C)(C)C)C3)c2)c2nccnc2c1)c1cccnc1]

### Intermediate 49

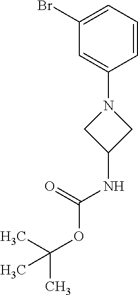 [SMILES: CC(C)(C)OC(=O)NC1CN(c2cccc(Br)c2)C1]

The product is prepared according to General Procedure 30, described in Example 74 with 1-bromo-3-iodobenzene (77.30 μl; 0.60 mmol; 1.30 eq.), azetidin-3-yl-carbamic acid tert-butyl ester (80.00 mg; 0.46 mmol; 1.00 eq.), NaOtBu (68.30 mg; 0.71 mmol; 1.53 eq.), Xantphos (80.63 mg; 0.14 mmol; 0.30 eq.), Pd₂(dba)₃ (42.54 mg; 0.05 mmol; 0.10 eq.) and toluene (3.00 mL). Purification by FCC (hexane/EtOAc; gradient). [1-(3-Bromophenyl)-azetidin-3-yl]-carbamic acid tert-butyl ester (150.00 mg; yield 64%; 65% by UPLC) is obtained as a beige solid.

### Intermediate 50

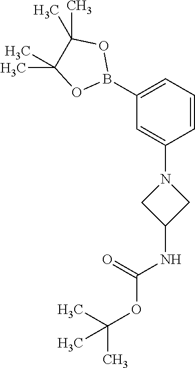 [SMILES: CC(C)(C)OC(=O)NC1CN(c2cccc(B3OC(C)(C)C(C)(C)O3)c2)C1]

The product is prepared according to General Procedure 41, described for Intermediate 39 with [1-(3-Bromo-phenyl)-azetidin-3-yl]-carbamic acid tert-butyl ester (Intermediate 49) (222.00 mg; 0.68 mmol; 1.00 eq.), bis(pina-colato)diborone (67.70 mg; 0.27 mmol; 1.10 eq.), KOAc (71.35 mg; 0.73 mmol; 3.00 eq.), Pd(dppf)Cl₂ (35.47 mg; 0.05 mmol; 0.20 eq.) and 1,4-dioxane (5.00 mL). RM is stirred at 80° C. for 20 h. Crude product {1-[3-(4,4,5,5-tetramethyl-[1,3,2]dioxaborolan-2-yl)-phenyl]-azetidin-3-yl}-carbamic acid tert-butyl ester (138.00 mg; yield 109.5%; 72% by UPLC) is used in the next step.

### Intermediate 51

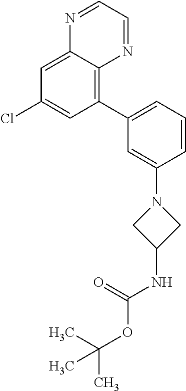 [SMILES: CC(C)(C)OC(=O)NC1CN(c2cccc(-c3cc(Cl)cc4nccnc34)c2)C1]

The product is prepared according to General Procedure 1, described for Intermediate 4 with 5-bromo-7-chloroquinoxaline (Intermediate 2) (50.00 mg; 0.20 mmol; 1.00 eq.), {1-[3-(4,4,5,5-tetramethyl-[1,3,2]dioxaborolan-2-yl)-phenyl]-azetidin-3-yl}-carbamic acid tert-butyl ester (Intermediate 50) (76.40 mg; 0.20 mmol; 1.00 eq.), DIPEA (70.82 μl; 0.41 mmol; 2.00 eq.), Pd(dppf)Cl₂ (14.87 mg; 0.02 mmol; 0.10 eq.), 1,4-dioxane (2.00 mL) and water (2.00 mL). Purification by FCC (hexane/EtOAc; gradient). {1-[3-(7-Chloroquinoxalin-5-yl)-phenyl]-azetidin-3-yl}-carbamic acid tert-butyl ester (49.00 mg; yield 56%; 96% by UPLC) is obtained as a yellow solid.

### Intermediate 52

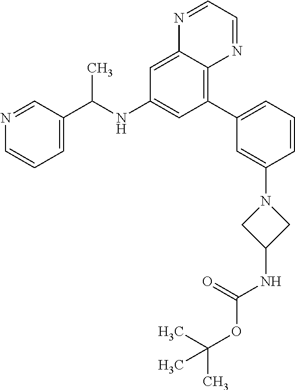 [SMILES: CC(Nc1cc(-c2cccc(N3CC(NC(=O)OC(C)(C)C)C3)c2)c2nccnc2c1)c1cccnc1]

The product is prepared according to General Procedure 2, described in Example 1 with {1-[3-(7-chloroquinoxalin-5-yl)-phenyl]-azetidin-3-yl}-carbamic acid tert-butyl ester (Intermediate 51) (49.00 mg; 0.12 mmol; 1.00 eq.), 1-pyridin-3-ylethylamine (28.29 μl; 0.24 mmol; 2.00 eq.), NaOtBu (34.38 mg; 0.36 mmol; 3.00 eq.), BINAP (14.85 mg; 0.02 mmol; 0.20 eq.), Pd₂(dba)₃ (10.92 mg; 0.01 mmol; 0.10 eq.) and toluene (2.50 mL). Purification by FCC (hexane/EtOAc; gradient and next EtOAc/MeOH; gradient). (1-{3-[7-(1-Pyridin-3-yl-ethylamino)-quinoxalin-5-yl]-phenyl}-azetidin-3-yl)-carbamic acid tert-butyl ester (21.00 mg; yield 35%; 100% by HPLC) is obtained as a yellow solid.

### Example 103

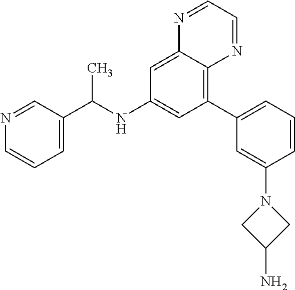 [SMILES: CC(Nc1cc(-c2cccc(N3CC(N)C3)c2)c2nccnc2c1)c1cccnc1]

The product is prepared according to General Procedure 10, described in Example 44 with (1-{3-[7-(1-Pyridin-3-yl-ethylamino)-quinoxalin-5-yl]-phenyl}-azetidin-3-yl)-carbamic acid tert-butyl ester (Intermediate 52) (20.00 mg; 0.04 mmol; 1.00 eq.), mixture of TFA in DCM and DCM (2.00 mL). 8-[3-(3-Aminoazetidin-1-yl)phenyl]-N-[1-(pyridin-3-yl)ethyl]quinoxalin-6-amine (15.00 mg; yield 92%; 98% by HPLC) is obtained as a light orange solid.

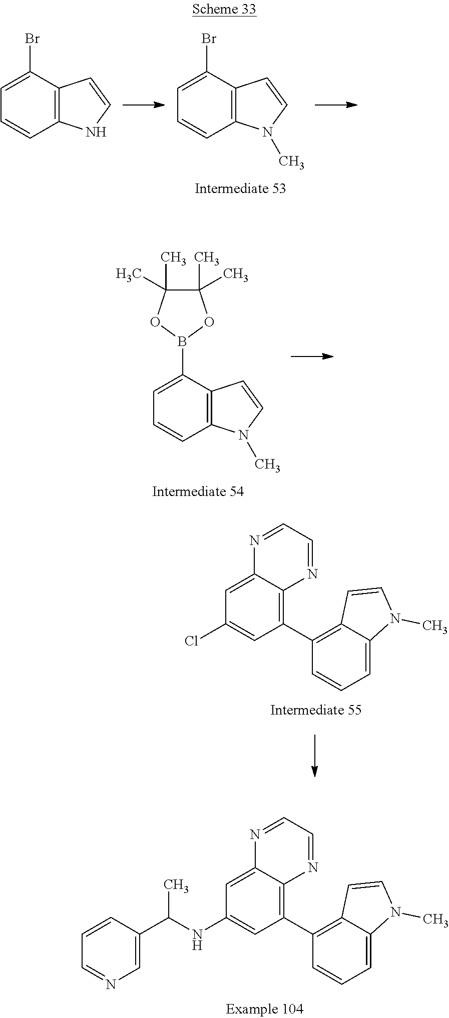 [SMILES: Brc1cccc2[nH]ccc12 ; Cn1ccc2c(Br)cccc21 ; Cn1ccc2c(B3OC(C)(C)C(C)(C)O3)cccc21 ; Cn1ccc2c(-c3cc(Cl)cc4nccnc34)cccc21 ; CC(Nc1cc(-c2cccc3c2ccn3C)c2nccnc2c1)c1cccnc1]

### Intermediate 53

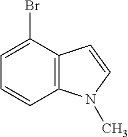

The product is prepared according to General Procedure 40, described for Intermediate 38 with 4-bromo-1H-indole (500.00 mg; 2.55 mmol; 1.00 eq.), NaH 60% in oil (204.04 mg; 5.10 mmol; 2.00 eq.), CH₃I (0.21 mL; 3.32 mmol; 1.30 eq.) and anhydrous DMF (5.00 mL). Crude product, 4-bromo-1-methyl-1H-indole (555.40 mg; yield 91%; 88% by UPLC) is used in the next step without purification.

### Intermediate 54

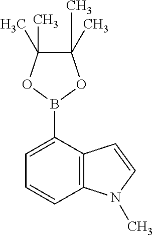 [SMILES: Cn1ccc2c(B3OC(C)(C)C(C)(C)O3)cccc21]

The product is prepared according to General Procedure 41, described for Intermediate 39 with 4-bromo-1-methyl-1H-indole (Intermediate 53) (250.00 mg; 1.06 mmol; 1.00 eq.), bis(pinacolato)diboron (349.65 mg; 1.38 mmol; 1.30 eq.), KOAc (207.90 mg; 2.12 mmol; 2.00 eq.), Pd(dppf)Cl₂ (7.75 mg; 0.01 mmol; 0.01 eq.) and 1,4-dioxane (20.00 mL). RM is stirred at 100° C. for 5H. Purification by FCC (hexane/EtOAc; gradient). 1-Methyl-4-(4,4,5,5-tetramethyl-[1,3,2]dioxaborolan-2-yl)-1H-indole (154.00 mg; yield 45%; 80% by UPLC) is obtained as a light yellow solid.

### Intermediate 55

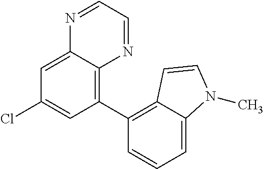 [SMILES: Cn1ccc2c(-c3cc(Cl)cc4nccnc34)cccc21]

The product is prepared according to General Procedure 1, described for Intermediate 4 with 5-bromo-7-chloroquinoxaline (Intermediate 2) (45.00 mg; 0.18 mmol; 1.00 eq.), 1-methyl-4-(4,4,5,5-tetramethyl-[1,3,2]dioxaborolan-2-yl)-1H-indole (Intermediate 54) (65.34 mg; 0.20 mmol; 1.10 eq.), DIPEA (0.06 mL; 0.37 mmol; 2.00 eq.), Pd(dppf)Cl₂*DCM (6.04 mg; 0.01 mmol; 0.04 eq.), 1,4-dioxane (2.00 mL) and water (2.00 mL). Crude product, 7-chloro-5-(1-methyl-1H-indol-4-yl)-quinoxaline (52.40 mg; yield 88%; 90% by UPLC) is used in the next step.

### Example 104

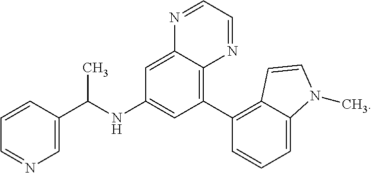 [SMILES: CC(Nc1cc(-c2cccc3c2ccn3C)c2nccnc2c1)c1cccnc1]

The product is prepared according to General Procedure 2, described in Example 1 with 7-chloro-5-(1-methyl-1H-indol-4-yl)-quinoxaline (Intermediate 55) (25.00 mg; 0.08 mmol; 1.00 eq.), 1-pyridin-3-yl-ethylamine (22.96 mg; 0.19 mmol; 2.40 eq.), NaOtBu (22.57 mg; 0.23 mmol; 3.00 eq.), BINAP (9.75 mg; 0.02 mmol; 0.20 eq.), Pd₂(dba)₃ (18.18 mg; 0.01 mmol; 0.10 eq.) and toluene (3.00 mL). Purification by FCC (DCM/MeOH; gradient). 8-(1-Methyl-1H-indol-4-yl)-N-[1-(pyridin-3-yl)ethyl]quinoxalin-6-amine (20.60 mg; yield 68%; 98% by HPLC) is obtained as a yellow powder.

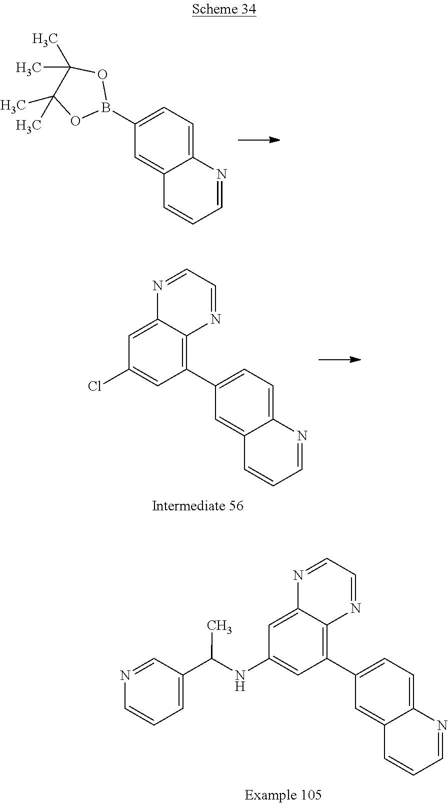

### Intermediate 56

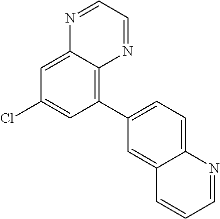 [SMILES: Clc1cc(-c2ccc3ncccc3c2)c2nccnc2c1]

The product is prepared according to General Procedure 39, described in Example 93 with 5-bromo-7-chloroquinoxaline (Intermediate 2) (450.00 mg; 1.77 mmol; 1.00 eq.), 6-(4,4,5,5-tetramethyl-[1,3,2]dioxaborolan-2-yl)-quinoline (466.64 mg; 1.77 mmol; 1.00 eq.), Cs₂CO₃ (1157.31 mg; 3.55 mmol; 2.00 eq.), Pd(dppf)2Cl2*DCM (221.77 mg; 0.27 mmol; 0.15 eq.), DME (10.00 mL) and water (5.00 mL). Purification by FCC (hexane/EtOAc; gradient). 7-Chloro-5-quinolin-6-yl-quinoxaline (337.00 mg; yield 64%; 94% by HPLC) is obtained as a beige powder.

### Example 105

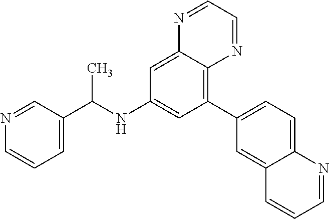 [SMILES: CC(Nc1cc(-c2ccc3ncccc3c2)c2nccnc2c1)c1cccnc1]

The product is prepared according to General Procedure 6, described for Intermediate 6 with 7-chloro-5-quinolin-6-yl-quinoxaline (Intermediate 56) (50.00 mg; 0.17 mmol; 1.00 eq.), 1-pyridin-3-yl-ethylamine (31.73 mg; 0.25 mmol; 1.50 eq.), Cs₂CO₃ (165.83 mg; 0.50 mmol; 3.00 eq.), BINAP (10.67 mg; 0.02 mmol; 0.10 eq.), Pd(OAc)₂ (3.97 mg; 0.02 mmol; 0.10 eq.) and 1,4-dioxane (2.00 mL). Purification by FCC (hexane/EtOAc; gradient). 1-Pyridin-3-yl-ethyl)-(8-quinolin-6-yl-quinoxalin-6-yl)-amine (46.00 mg; yield 68%; 94% by HPLC) is obtained as a yellow powder.

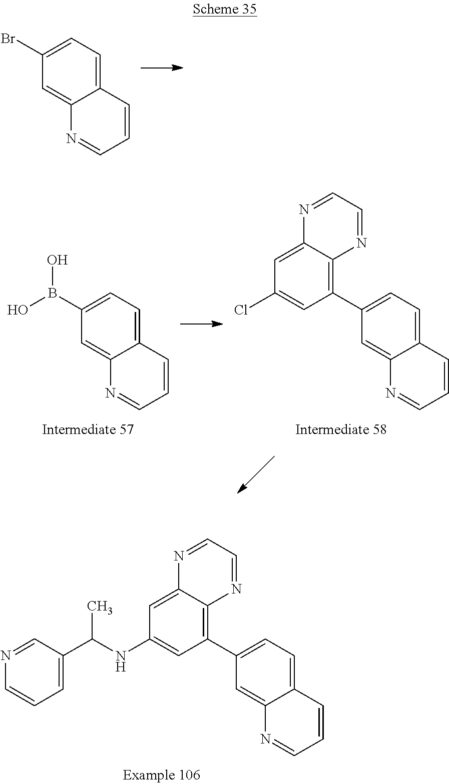 [SMILES: Brc1ccc2cccnc2c1 ; OB(O)c1ccc2cccnc2c1 ; Clc1cc(-c2ccc3cccnc3c2)c2nccnc2c1 ; CC(Nc1cc(-c2ccc3cccnc3c2)c2nccnc2c1)c1cccnc1]

### Intermediate 57

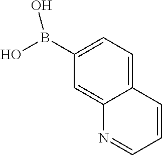 [SMILES: OB(O)c1ccc2cccnc2c1]

The product is prepared according to General Procedure 41, described for Intermediate 39 with 7-bromoquinoline (300.00 mg; 1.44 mmol; 1.00 eq.), bis(pinacolato)diboron (439.39 mg; 1.73 mmol; 1.20 eq.), Pd(dppf)Cl₂ (52.75 mg; 0.07 mmol; 0.05 eq.), KOAc (424.54 mg; 4.33 mmol; 3.00 eq.) and anhydrous 1,4-dioxane (5.00 mL). After extraction 7-quinolylboronic acid (175.00 mg; yield 55%; 78% by UPLC) is used in the next step.

### Intermediate 58

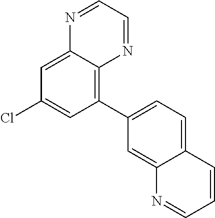 [SMILES: Clc1cc(-c2ccc3cccnc3c2)c2nccnc2c1]

The product is prepared according to General Procedure 1, described for Intermediate 4 with 5-bromo-7-chloroquinoxaline (Intermediate 2) (387.00 mg; 1.59 mmol; 1.00 eq.), 7-quinolylboronic acid (Intermediate 57) (175.00 mg; 0.79 mmol; 1.00 eq.), DIPEA (0.56 mL; 3.18 mmol; 2.00 eq.), 1,4-dioxane (1.50 mL) and water (1.50 mL). Purification by FCC (hexane/EtOAc; gradient). 7-Chloro-5-quinolin-7-yl-quinoxaline (56.00 mg; yield 22%; 91% by UPLC) is obtained as a light yellow solid.

### Example 106

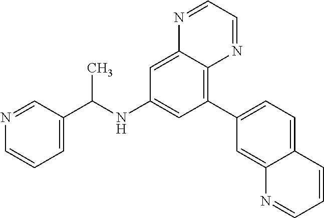 [SMILES: CC(Nc1cc(-c2ccc3cccnc3c2)c2nccnc2c1)c1cccnc1]

The product is prepared according to General Procedure 2, described in Example 1 with 7-chloro-5-quinolin-7-yl-quinoxaline (Intermediate 58) (50.00 mg; 0.17 mmol; 1.00 eq.), 1-pyridin-3-yl-ethylamine (41.88 mg; 0.34 mmol; 2.00 eq.), NaOtBu (49.36 mg; 0.51 mmol; 3.00 eq.), BINAP (21.34 mg; 0.03 mmol; 0.20 eq.), Pd₂(dba)₃ (15.69 mg; 0.02 mmol; 0.10 eq.) and toluene (2.00 mL). Purification by FCC (DCM/MeOH; gradient). (1-Pyridin-3-yl-ethyl)-(8-quinolin-7-yl-quinoxalin-6-yl)-amine (17.00 mg; yield 25%; 95% by HPLC) is obtained as a yellow solid.

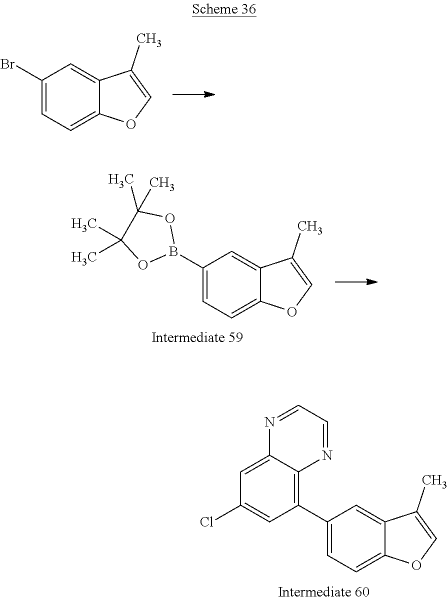 [SMILES: Cc1coc2ccc(Br)cc12 ; Cc1coc2ccc(B3OC(C)(C)C(C)(C)O3)cc12 ; Cc1coc2ccc(-c3cc(Cl)cc4nccnc34)cc12]

### Intermediate 59

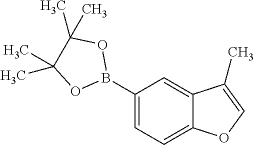 [SMILES: Cc1coc2ccc(B3OC(C)(C)C(C)(C)O3)cc12]

The product is prepared according to General Procedure 41, described for Intermediate 39 with 5-bromo-3-methylbenzofuran (150.00 mg; 0.71 mmol; 1.00 eq.), bis(pinacolato)diboron (216.57 mg; 0.85 mmol; 1.20 eq.), KOAc (209.25 mg; 2.13 mmol; 3.00 eq.), Pd(dppf)Cl₂ (52.00 mg; 0.07 mmol; 0.10 eq.) and 1,4-dioxane (4.00 mL). Purification by FCC (hexane/EtOAc: gradient). 3-Methyl-5-(4,4,5,5-tetramethyl-[1,3,2]dioxaborolan-2-yl)-benzofuran (409.00 mg; yield 73%; 83% by UPLC) is obtained as a brown solid.

### Intermediate 60

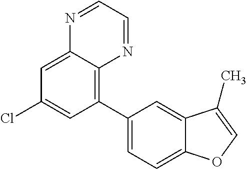 [SMILES: Cc1coc2ccc(-c3cc(Cl)cc4nccnc34)cc12]

The product is prepared according to General Procedure 39, described for Example 93 with 5-bromo-7-chloroquinoxaline (Intermediate 2) (360.00 mg; 1.42 mmol; 1.00 eq.), 3-methyl-5-(4,4,5,5-tetramethyl-[1,3,2]dioxaborolan-2-yl)-benzofuran (Intermediate 59) (385.65 mg; 1.42 mmol; 1.00 eq.), Cs₂CO₃ (924.92 mg; 2.84 mmol; 2.00 eq.), Pd(dppf)₂Cl₂*DCM (173.87 mg; 0.21 mmol; 0.15 eq.), DME (15.00 mL) and water (5.00 mL) is added. Purification by FCC (hexane/EtOAc; gradient). 7-Chloro-5-(3-methylbenzofuran-5-yl)-quinoxaline (374.00 mg; yield 65%; 73% by UPLC) is obtained as a yellow solid.

### Example 107

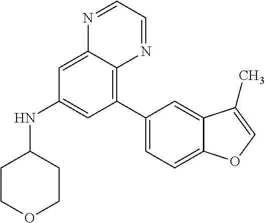 [SMILES: Cc1coc2ccc(-c3cc(NC4CCOCC4)cc4nccnc34)cc12]

The product is prepared according to General Procedure 2, described in Example 1 with 7-chloro-5-(3-methylbenzofuran-5-yl)-quinoxaline (Intermediate 46) (40.00 mg; 0.14 mmol; 1.00 eq.), tetrahydropyran-4-ylamine (16.47 mg; 0.16 mmol; 1.20 eq.), NaOtBu (31.30 mg; 0.33 mmol; 2.40 eq.), BINAP (8.45 mg; 0.01 mmol; 0.10 eq.), Pd₂(dba)₃ (15.75 mg; 0.01 mmol; 0.05 eq.) and toluene (3.00 mL). RM is stirred at 120° C. for 17 h. Purification by FCC (DCM/MeOH; gradient, NH₂ column). 8-(3-Methyl-1-benzofuran-5-yl)-N-(oxan-4-yl)quinoxalin-6-amine (11.00 mg; yield 22%; 97% by HPLC) is obtained as a yellow powder.

### Example 108

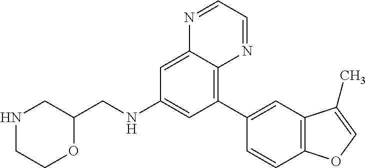 [SMILES: Cc1coc2ccc(-c3cc(NCC4CNCCO4)cc4nccnc34)cc12]

The product is prepared according to General Procedure 5, described in Example 30 with 7-chloro-5-(3-methylbenzofuran-6-yl)quinoxaline (Intermediate 60) (50.0 mg, 0.71 mmol; 1.0 eq.), C-morpholin-2-ylmethylamine. 24.63 mg; 0.21 mmol; 1.25 eq.), NaOtBu (22.82 mg; 0.24 mmol; 1.40 eq.), [(Cinnamyl)PdCl]₂ (4.39 mg; 0.01 mmol; 0.05 eq.), BippyPhos (6.88 mg; 0.01 mmol; 0.08 eq.) and anhydrous toluene (5.00 mL). Purification by FCC (EtOAc/DCM/MeOH; gradient). [8-(3-Methylbenzofuran-6-yl)quinoxalin-6-yl]morpholin-2-ylmethylamine (12.60 mg; yield 18%; 92% by HPLC) is obtained as a yellow powder.

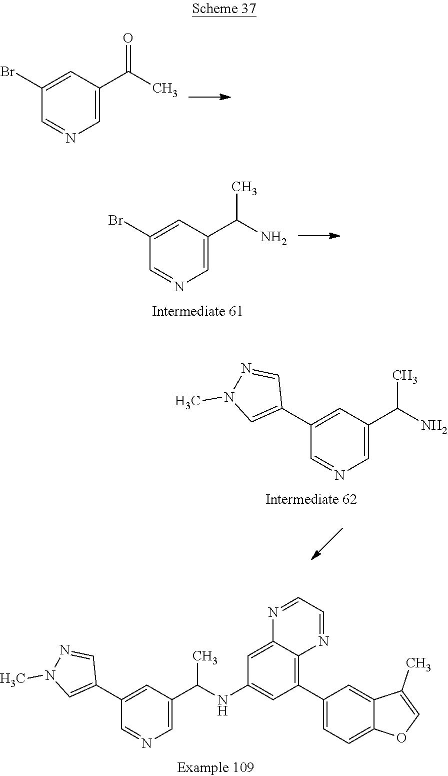

### Intermediate 61

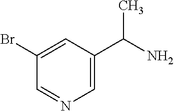

The product is prepared according to General Procedure 14, described for Intermediate 12 with 1-(5-bromopyridin-3-yl)-ethanone (400.00 mg; 2.00 mmol; 1.00 eq.), TTIP (1.18 mL; 4.00 mmol; 2.00 eq.), NaBH₄ (151.31 mg; 4.00 mmol; 2.00 eq.) and 7M NH₃ in MeOH (5.00 mL). After extraction 1-(5-bromopyridin-3-yl)-ethylamine (402.00 mg; yield 94%; 91% by UPLC) is directly used in the next step without further purification.

### Intermediate 62

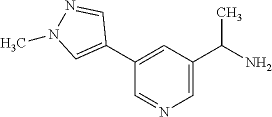

The product is prepared according to General Procedure 29, described in Example 72 with 1-(5-bromopyridin-3-yl)-ethylamine (Intermediate 61) (80.00 mg; 0.37 mmol; 1.00 eq.), 1-methyl-4-(4,4,5,5-tetramethyl-[1,3,2]dioxaborolan-2-yl)-1H-pyrazole (155.64 mg; 0.75 mmol; 2.00 eq.), KOAc (220.24 mg; 2.24 mmol; 6.00 eq.), Pd(dppf)Cl₂ (68.42 mg; 0.09 mmol; 0.25 eq.) and CH₃CN (2.00 mL) and water (1.00 mL). 1-[5-(1-Methyl-1H-pyrazol-4-yl)-pyridin-3-yl]-ethylamine (46.00 mg; yield 56%; 90% by UPLC) is used in the next step, without further purification.

### Example 109

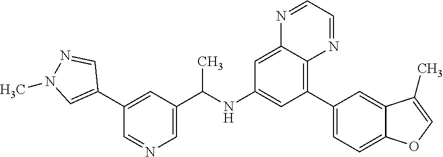 [SMILES: Cc1coc2ccc(-c3cc(NC(C)c4cncc(-c5cnn(C)c5)c4)cc4nccnc34)cc12]

The product is prepared according to General Procedure 2, described in Example 1 with 7-chloro-5-(3-methyl-benzofuran-5-yl)-quinoxaline (35.00 mg; 0.12 mmol; 1.00 eq.) (Intermediate 60), 1-[5-(1-methyl-1H-pyrazol-4-yl)-pyridin-3-yl]-ethylamine (Intermediate 62) (44.34 mg; 0.14 mmol; 1.20 eq.), NaOtBu (27.39 mg; 0.29 mmol; 2.40 eq.), BINAP (7.39 mg; 0.01 mmol; 0.10 eq.), Pd₂(dba)₃ (54.37 mg; 0.06 mmol; 0.50 eq.) and toluene (3.00 mL). Purification by FCC (hexane/EtOAc; gradient). 8-(3-Methyl-1-benzofuran-5-yl)-N-{1-[5-(1-methyl-1H-pyrazol-4-yl)pyridin-3-yl]ethyl}quinoxalin-6-amine (6.00 mg; yield 10%; 96% by HPLC) is obtained as a yellow powder.

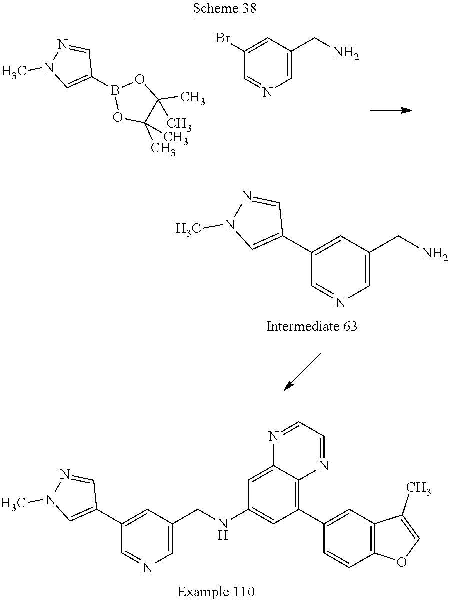

### Intermediate 63

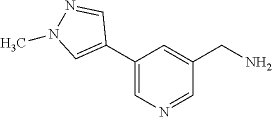

The product is prepared according to General Procedure 29, described in Example 72 with C-(5-bromopyridin-3-yl)-methylamine (80.00 mg; 0.40 mmol; 1.00 eq.), 1-methyl-4-(4,4,5,5-tetramethyl-[1,3,2]dioxaborolan-2-yl)-1H-pyrazole (167.31 mg; 0.80 mmol; 2.00 eq.), KOAc (236.75 mg; 2.41 mmol; 6.00 eq.), CH₃CN (2.00 mL) and water (1.00 mL). After extraction crude C-[5-(1-methyl-1H-pyrazol-4-yl)-pyridin-3-yl]-methylamine (85.00 mg; yield 88%; 78% by UPLC) is used in the next step.

### Example 110

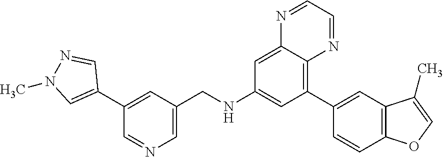 [SMILES: Cc1coc2ccc(-c3cc(NCc4cncc(-c5cnn(C)c5)c4)cc4nccnc34)cc12]

The product is prepared according to General Procedure 2, described in Example 1 with 7-chloro-5-(3-methylbenzofuran-5-yl)-quinoxaline (Intermediate 60) (45.00 mg; 0.15 mmol; 1.00 eq.), C-[5-(1-methyl-1H-pyrazol-4-yl)-pyridin-3-yl]-methylamine (Intermediate 63) (84.01 mg; 0.35 mmol; 2.40 eq.), NaOtBu (41.82 mg; 0.44 mmol; 3.00 eq.), BINAP (18.06 mg; 0.03 mmol; 0.20 eq.), Pd₂(dba)₃ (13.28 mg; 0.01 mmol; 0.10 eq.) and toluene (3.00 mL). Purification by FCC (NH₂ column; DCM/MeOH; gradient). [8-(3-Methylbenzofuran-5-yl)-quinoxalin-6-yl]-[5-(1-methyl-1H-pyrazol-4-yl)-pyridin-3-ylmethyl]-amine (17.70 mg; yield 25%; 93% by HPLC) is obtained as a yellow powder.

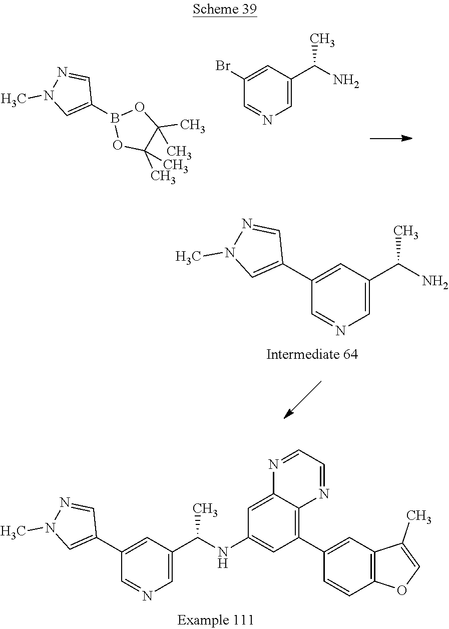

### Intermediate 64—General Procedure 44

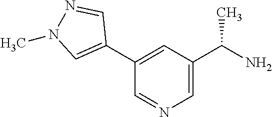

A sealed tube is charged with (S)-1-(3-Bromo-phenyl)-ethylamine (100.00 mg; 0.50 mmol; 1.00 eq.), 1-methyl-4-(4,4,5,5-tetramethyl-[1,3,2]dioxaborolan-2-yl)-1H-pyrazole (109.19 mg; 0.52 mmol; 1.05 eq.), Na₂CO₃ (52.97 mg; 0.5 mmol; 1.0 eq.), CH₃CN (1.5 mL) and water (0.5 mL). The suspension is purged with argon and then Pd(PPh₃)₄ (28.88 mg; 0.02 mmol; 0.05 eq.) is added. The tube is sealed and reaction is carried out in a MW reactor at 100° C. for 30 min. After this time, the mixture is filtered through a Celite® pad and the filtrate is diluted with EtOAc and extracted with water. Organic phase is washed with brine, dried over Na₂SO₄ and then solvent is evaporated. Crude (S)-1-[3-(1-methyl-1H-pyrazol-4-yl)-phenyl]-ethylamine (80 mg, yield 80%, 96% by UPLC) is used in the next step.

### Example 111

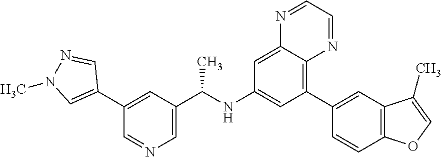 [SMILES: Cc1coc2ccc(-c3cc(N[C@@H](C)c4cncc(-c5cnn(C)c5)c4)cc4nccnc34)cc12]

The product is prepared according to General Procedure 2, described in Example 1 with 7-chloro-5-(3-methylbenzofuran-5-yl)-quinoxaline (Intermediate 60) (40.00 mg; 0.13 mmol; 1.00 eq.), (S)-1-[3-(1-methyl-1H-pyrazol-4-yl)-phenyl]-ethylamine (Intermediate 64) (63.86 mg; 0.31 mmol; 2.40 eq.), NaOtBu (36.78 mg; 0.38 mmol; 3.00 eq.), BINAP (15.89 mg; 0.03 mmol; 0.20 eq.), Pd2(dba)3 (11.68 mg; 0.01 mmol; 0.10 eq.) and toluene (3.00 mL). Purification by FCC (hexane/EtOAc; gradient). [8-(3-Methyl-benzofuran-5-yl)-quinoxalin-6-yl]-{(S)-1-[3-(1-methyl-1H-pyrazol-4-yl)-phenyl]-ethyl}-amine (11.50 mg; yield 18%; 94% by HPLC) is obtained as a yellow powder.

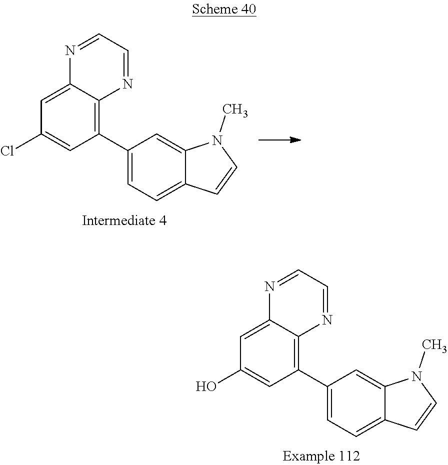 [SMILES: Cn1ccc2ccc(-c3cc(Cl)cc4nccnc34)cc21 ; Cn1ccc2ccc(-c3cc(O)cc4nccnc34)cc21]

### Example 112—General Procedure 45

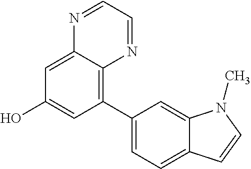 [SMILES: Cn1ccc2ccc(-c3cc(O)cc4nccnc34)cc21]

A pressure vessel is charged with 7-chloro-5-(1-methyl-1H-indol-6-yl)-quinoxaline (Intermediate 4) (50.00 mg; 0.16 mmol; 1.00 eq.), K₂CO₃ (66.34 mg; 0.48 mmol; 3.00 eq.), tBuXPhos (10.87 mg; 0.03 mmol; 0.16 eq.), DMF (2 mL) and water (2 mL). RM is purged with argon, then Herrmann's catalyst (6.00 mg; 0.01 mmol; 0.04 eq.) is added. RM is sealed and stirred at MW at 115° C. for 30 minutes. Solvent is evaporated and product is purified by FCC (hexane/EtOAc; gradient). 8-(1-Methyl-1H-indol-6-yl)-quinoxalin-6-ol (38.00 mg; yield 83%; 96% by HPLC) is obtained as an orange solid.

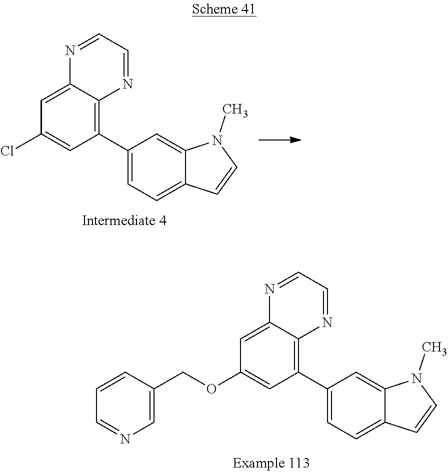 [SMILES: Cn1ccc2ccc(-c3cc(Cl)cc4nccnc34)cc21 ; Cn1ccc2ccc(-c3cc(OCc4cccnc4)cc4nccnc34)cc21]

### Example 113—General Procedure 46

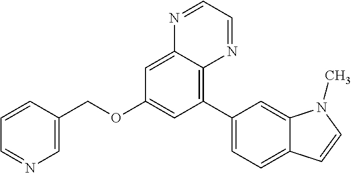 [SMILES: Cn1ccc2ccc(-c3cc(OCc4cccnc4)cc4nccnc34)cc21]

A sealed tube is charged with 7-chloro-5-(1-methyl-1H-indol-6-yl)-quinoxaline (Intermediate 4) (50.00 mg; 0.15 mmol; 1.00 eq.), pyridin-3-ylmethanol (33.43 mg; 0.31 mmol; 2.00 eq.), BrettPhos (5.76 mg; 0.01 mmol; 0.07 eq.), NaOtBu (20.61 mg; 0.21 mmol; 1.40 eq.), BrettPhos precatalyst (8.57 mg; 0.01 mmol; 0.07 eq.) and 1,4-dioxane (3.00 mL). It is sealed, degassed and purged with argon. RM is stirred at 100° C. for 4 h. Solvent is evaporated and the residue is purified by FCC (hexane/EtOAc; gradient, then EtOAc/MeOH; gradient). 5-(1-Methyl-1H-indol-6-yl)-7-(pyridin-3-ylmethoxy)quinoxaline (17.80 mg; yield 30%; 93% by HPLC) is obtained as a yellow powder.

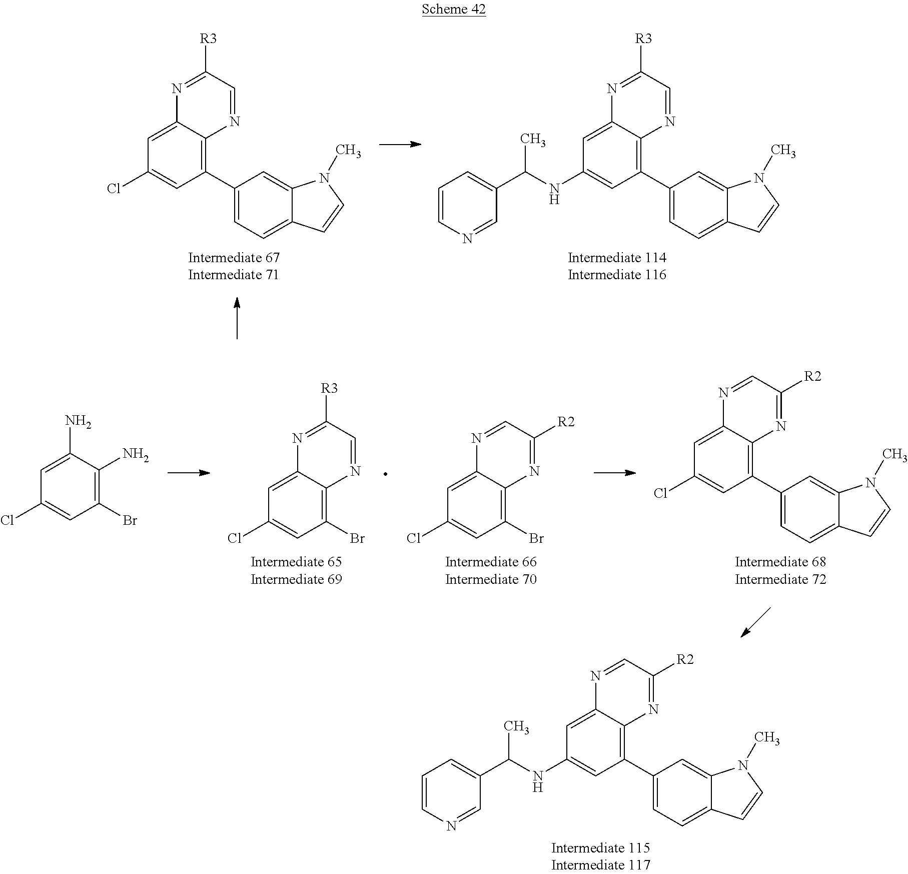

### Intermediate 65 & 66—General Procedure 47

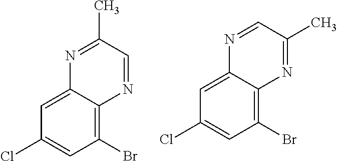

A sealed tube is charged with 3-bromo-5-chlorobenzene-1,2-diamine (Intermediate 1) (0.50 g; 2.19 mmol; 1.00 eq.), 2-oxopropionaldehyde (0.33 mL; 2.19 mmol; 1.00 eq.) and water (1.00 mL). RM is sealed and stirred at 40° C. for 2 h. Solvent is evaporated and mixture of desired products is separated by FCC (hexane/EtOAc; gradient). 5-bromo-7-chloro-2-methyl-quinoxaline (Intermediate 65) (115.00 mg; yield 20%; 98% by UPLC) and 8-bromo-6-chloro-2-methyl-quinoxaline (Intermediate 66) (320.00 mg; yield 56%; 97% by UPLC) are obtained as a white amorphous foams.

### Intermediate 65

¹H NMR (400 MHz, DMSO) δ 8.97 (s, 1H), 8.27 (d, J=2.2 Hz, 1H), 8.14 (d, J=2.2 Hz, 1H), 2.75 (s, 3H).

### Intermediate 66

¹H NMR (400 MHz, DMSO) δ 8.95 (s, 1H), 8.30 (d, J=2.2 Hz, 1H), 8.19 (d, J=2.3 Hz, 1H), 2.76 (s, 3H).

### Intermediate 67

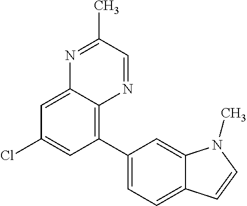 [SMILES: Cc1cnc2c(-c3ccc4ccn(C)c4c3)cc(Cl)cc2n1]

The product is prepared according to General Procedure 1, described for Intermediate 4 with 5-bromo-7-chloro-2-methylquinoxaline (Intermediate 65) (85.00 mg; 0.33 mmol; 1.00 eq.), 1-methyl-6-(4,4,5,5-tetramethyl-[1,3,2]dioxaborolan-2-yl)-1H-indole (93.36 mg; 0.36 mmol; 1.10 eq.), DIPEA (0.11 mL; 0.66 mmol; 2.00 eq.), Pd(dppf)Cl₂ (24.14 mg; 0.03 mmol; 0.10 eq.) and 1,4-dioxane (7.00 mL). Purification by FCC (hexane/EtOAc, gradient). 7-Chloro-2-methyl-5-(1-methyl-1H-indol-6-yl)-quinoxaline (64.00 mg; yiled 61%; 97% by HPLC) is obtained as a yellow solid.

¹H NMR (400 MHz, DMSO) δ 8.87 (s, 1H), 8.05 (d, J=2.4 Hz, 1H), 7.86 (d, J=2.4 Hz, 1H), 7.76-7.72 (m, 1H), 7.63 (dd, J=8.2, 0.6 Hz, 1H), 7.42 (d, J=3.0 Hz, 1H), 7.35 (dd, J=8.2, 1.5 Hz, 1H), 6.49 (dd, J=3.1, 0.8 Hz, 1H), 3.84 (s, 3H), 2.73 (s, 3H).

### Intermediate 68

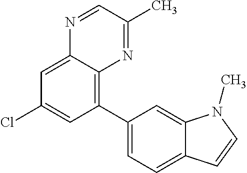 [SMILES: Cc1cnc2cc(Cl)cc(-c3ccc4ccn(C)c4c3)c2n1]

The product is prepared according to General Procedure 1, described for Intermediate 4 with 8-bromo-6-chloro-2-methylquinoxaline (Intermediate 66) (85.00 mg; 0.33 mmol; 1.00 eq.), 1-methyl-6-(4,4,5,5-tetramethyl-[1,3,2]dioxaborolan-2-yl)-1H-indole (54.92 mg; 0.21 mmol; 1.10 eq.), DIPEA (0.07 mL; 0.39 mmol; 2.00 eq.), Pd(dppf)Cl₂ (14.20 mg; 0.02 mmol; 0.10 eq.) and 1,4-dioxane (7.00 mL). Purification by FCC (hexane/EtOAc; gradient). 6-Chloro-2-methyl-8-(1-methyl-1H-indol-6-yl)-quinoxaline (55.00 mg; yield 89%; 97% by HPLC) is obtained as a red amorphous solid.

¹H NMR (400 MHz, DMSO) δ 8.87 (s, 1H), 8.05 (d, J=2.4 Hz, 1H), 7.86 (d, J=2.4 Hz, 1H), 7.76-7.72 (m, 1H), 7.63 (dd, J=8.2, 0.6 Hz, 1H), 7.42 (d, J=3.0 Hz, 1H), 7.35 (dd, J=8.2, 1.5 Hz, 1H), 6.49 (dd, J=3.1, 0.8 Hz, 1H), 3.84 (s, 3H), 2.73 (s, 3H).

### Example 114

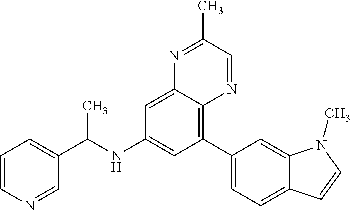 [SMILES: Cc1cnc2c(-c3ccc4ccn(C)c4c3)cc(NC(C)c3cccnc3)cc2n1]

The product is prepared according to General Procedure 2, described in Example 1 with 7-chloro-2-methyl-5-(1-methyl-1H-indol-6-yl)-quinoxaline (Intermediate 67) (48.00 mg; 0.15 mmol; 1.00 eq.), 1-pyridin-3-yl-ethylamine (43.44 mg; 0.36 mmol; 2.40 eq.), NaOtBu (42.72 mg; 0.44 mmol; 3.00 eq.), BINAP (18.45 mg; 0.03 mmol; 0.20 eq.), Pd₂(dba)₃ (34.39 mg; 0.01 mmol; 0.10 eq.) and toluene (4.00 mL). RM is carried out in a MW reactor at 160° C. for 1 h. Purification by FCC (DCM/MeOH; gradient). 3-methyl-8-(1-methyl-1H-indol-6-yl)-N-[1-(pyridin-3-yl)ethyl]quinoxalin-6-amine (21.50 mg; yield 33%; 91% by HPLC) is obtained as an orange solid.

### Example 115

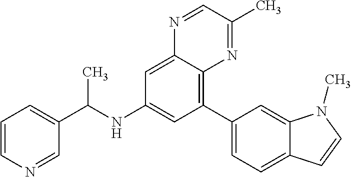 [SMILES: Cc1cnc2cc(NC(C)c3cccnc3)cc(-c3ccc4ccn(C)c4c3)c2n1]

The product is prepared according to General Procedure 2, described in Example 1 with 6-chloro-2-methyl-8-(1-methyl-1H-indol-6-yl)-quinoxaline (Intermediate 68) (41.00 mg; 0.13 mmol; 1.00 eq.), 1-pyridin-3-yl-ethylamine (37.11 mg; 0.30 mmol; 2.40 eq.), NaOtBu (36.49 mg; 0.38 mmol; 3.00 eq.), BINAP (15.76 mg; 0.03 mmol; 0.20 eq.), Pd₂(dba)₃ (29.38 mg; 0.01 mmol; 0.10 eq.) and toluene (4.00 mL). RM is carried out in a MW reactor at 160° C. for 1 h. Purification by FCC (DCM/MeOH; gradient). 2-Methyl-8-(1-methyl-1H-indol-6-yl)-N-[1-(pyridin-3-yl)ethyl]quinoxalin-6-amine (31.00 mg; yield 59%; 95% by HPLC) is obtained as a yellow amorphous solid.

### Intermediate 69 & 70—General Procedure 48

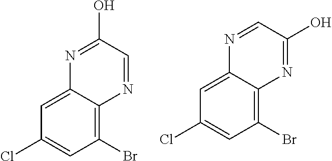

A sealed tube is charged with 3-bromo-5-chlorobenzene-1,2-diamine (Intermediate 1) (1.00 g; 4.38 mmol; 1.00 eq.), ethyl glyoxalate (0.94 mL; 4.60 mmol; 1.05 eq.) and EtOH (40.00 mL). RM is sealed and stirred at 40° C. for 2h. Solvent is evaporated and product is purified by FCC (hexane/EtOAc; gradient). Product (767.00 mg; yield 67%; 99% by UPLC) is obtained as a mixture of both isomers: 5-bromo-7-chloroquinoxalin-2-ol (Intermediate 69) and 8-bromo-6-chloroquinoxalin-2-ol (Intermediate 70).

### Intermediate 71 & 72

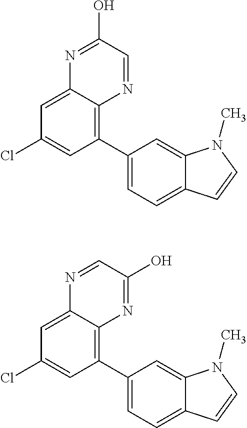 [SMILES: Cn1ccc2ccc(-c3cc(Cl)cc4nc(O)cnc34)cc21 ; Cn1ccc2ccc(-c3cc(Cl)cc4ncc(O)nc34)cc21]

The product is prepared according to General Procedure 1, described for Intermediate 4 with 5-bromo-7-chloroquinoxalin-2-ol (Intermediate 69) and 8-bromo-6-chloroquinoxalin-2-ol (Intermediate 70) (520.00 mg; 2.00 mmol; 1.00 eq.), 1-methyl-6-(4,4,5,5-tetramethyl-[1,3,2]dioxaborolan-2-yl)-1H-indole (566.82 mg; 2.20 mmol; 1.10 eq.), DIPEA (0.70 mL; 4.01 mmol; 2.00 eq.), Pd(dppf)Cl2 (146.57 mg; 0.20 mmol; 0.10 eq.) and 1,4-dioxane (7.00 mL). Separation by FCC (hexane/EtOAc; gradient). 7-Chloro-5-(1-methyl-1H-indol-6-yl)-quinoxalin-2-ol (Intermediate 71) (154.00 mg; yield 18.6; 75% by UPLC) and 6-Chloro-8-(1-methyl-1H-indol-6-yl)-quinoxalin-2-ol (Intermediate 72) (238.00 mg; yield 35.7%; 91% by UPLC) are obtained as a yellow solids.

### Example 116

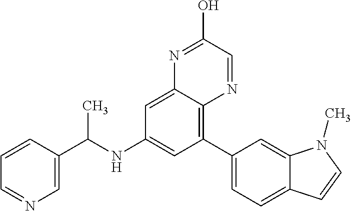 [SMILES: CC(Nc1cc(-c2ccc3ccn(C)c3c2)c2ncc(O)nc2c1)c1cccnc1]

The product is prepared according to General Procedure 3, described in Example 18 with 7-chloro-5-(1-methyl-1H-indol-6-yl)-quinoxalin-2-ol (Intermediate 71) (40.00 mg; 0.12 mmol; 1.00 eq.), 1-pyridin-3-ylethylamine (0.02 mL; 0.14 mmol; 1.20 eq.), BrettPhos (4.37 mg; 0.01 mmol; 0.07 eq.), BrettPhos precatalyst (6.50 mg; 0.01 mmol; 0.07 eq.) and LiHMDS 1.0 M in THF solution (0.28 mL; 0.28 mmol; 2.40 eq.). RM is stirred at 65° C. for 5 h. Purification by FCC (hexane/EtOAc; gradient; silica gel deactivated with NH₃). 5-(1-Methyl-1H-indol-6-yl)-7-{[1-(pyridin-3-yl)ethyl]amino}quinoxalin-2-ol (14.00 mg; yield 30%; 98% by HPLC) is obtained as a yellow powder.

### Example 117

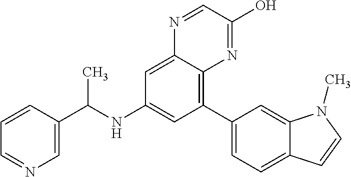 [SMILES: CC(Nc1cc(-c2ccc3ccn(C)c3c2)c2nc(O)cnc2c1)c1cccnc1]

The product is prepared according to General Procedure 3, described in Example 18 with 6-chloro-8-(1-methyl-1H-indol-6-yl)-quinoxalin-2-ol (Intermediate 72) (45.00 mg; 0.14 mmol; 1.00 eq.), 1-pyridin-3-ylethylamine (0.02 mL; 0.17 mmol; 1.20 eq.), BrettPhos (5.45 mg; 0.01 mmol; 0.07 eq.), BrettPhos precatalyst (8.11 mg; 0.01 mmol; 0.07 eq.) and LiHMDS 1.0 M in THF solution (347.97 μl; 0.35 mmol; 2.40 eq.). RM is stirred at 65° C. for 5 h. Purification by FCC (hexane/EtOAc; gradient; silica gel deactivated with NH₃(aq)). 8-(1-Methyl-1H-indol-6-yl)-6-{[1-(pyridin-3-yl)ethyl]amino}quinoxalin-2-ol (39.80 mg; yield 68%; 99% by HPLC) is obtained as an orange powder.

### Example 118

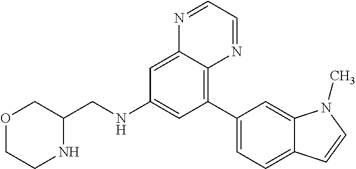 [SMILES: Cn1ccc2ccc(-c3cc(NCC4COCCN4)cc4nccnc34)cc21]

The product is prepared according to General Procedure 5, described in Example 30 with 7-chloro-5-(1-methyl-1H-indol-6-yl)-quinoxaline (Intermediate 4) (75.00 mg; 0.26 mmol; 1.00 eq.), C-morpholin-3-yl-methylamine (70.85 mg; 0.61 mmol; 2.40 eq.), NaOtBu (34.35 mg; 0.36 mmol; 1.40 eq.), [(Cinnamyl)PdCl]₂ (6.61 mg; 0.01 mmol; 0.05 eq.), BippyPhos (10.35 mg; 0.02 mmol; 0.08 eq.) and toluene anhydrous (5.00 mL). Purification by FCC (hexane/EtOAc; gradient; and next EtOAc/MeOH; gradient). [8-(1-Methyl-1H-indol-6-yl)-quinoxalin-6-yl]-morpholin-3-ylmethyl-amine (45.00 mg; yield 44.5%; 94% by HPLC) is obtained as a yellow powder.

### Example 119

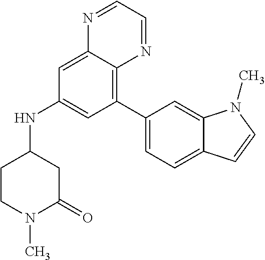 [SMILES: CN1CCC(Nc2cc(-c3ccc4ccn(C)c4c3)c3nccnc3c2)CC1=O]

The product is prepared according to General Procedure 2, described in Example 1 with 7-chloro-5-(1-methyl-1H-indol-6-yl)-quinoxaline (Intermediate 4) (60.00 mg; 0.20 mmol; 1.00 eq.), 4-amino-1-methyl-piperidin-2-one hydrochloride (39.95 mg; 0.24 mmol; 1.20 eq.), NaOtBu (58.30 mg; 0.61 mmol; 3.00 eq.), Pd₂(dba)₃ (18.52 mg; 0.02 mmol; 0.10 eq.), BINAP (25.18 mg; 0.04 mmol; 0.20 eq.) and toluene (2.50 mL). Purification by FCC (DCM/MeOH; gradient). 1-Methyl-4-[8-(1-methyl-1H-indol-6-yl)-quinoxalin-6-ylamino]-piperidin-2-one (46.00 mg; yield 55%; 93% by HPLC) is obtained as a yellow solid.

### Example 120

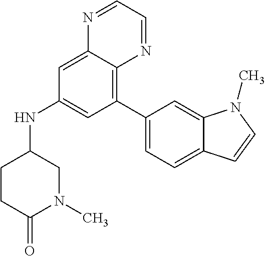 [SMILES: CN1CC(Nc2cc(-c3ccc4ccn(C)c4c3)c3nccnc3c2)CCC1=O]

The product is prepared according to General Procedure 3, described in Example 18 with 7-chloro-5-(1-methyl-1H-indol-6-yl)-quinoxaline (Intermediate 4) (56.00 mg; 0.19 mmol; 1.00 eq.), 5-amino-1-methylpiperidin-2-one (26.61 mg; 0.21 mmol; 1.10 eq.), BrettPhos (5.07 mg; 0.01 mmol; 0.05 eq.), BrettPhos precatalyst (7.54 mg; 0.01 mmol; 0.05 eq.) and LiHMDS 1.0 M in THF (339.72 μl; 0.34 mmol; 1.80 eq.). Purification by FCC (hexane/EtOAc; gradient). 1-Methyl-5-[8-(1-methyl-1H-indol-6-yl)-quinoxalin-6-ylamino]-piperidin-2-one (25.00 mg; yield 34%; 99% by HPLC) is obtained as a yellow solid.

### Example 121

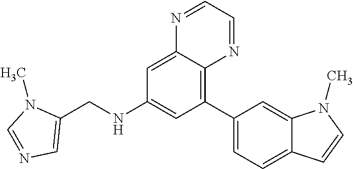 [SMILES: Cn1cncc1CNc1cc(-c2ccc3ccn(C)c3c2)c2nccnc2c1]

The product is prepared according to General Procedure 6, described for Intermediate 6 with 7-chloro-5-(1-methyl-1H-indol-6-yl)-quinoxaline (Intermediate 4) (60.00 mg; 0.20 mmol; 1.00 eq.), C-(3-methyl-3H-imidazol-4-yl)-methylamine (33.37 mg; 0.30 mmol; 1.50 eq.), Cs₂CO₃ (197.63 mg; 0.60 mmol; 3.00 eq.), BINAP (12.72 mg; 0.02 mmol; 0.10 eq.), Pd(OAc)₂ (4.73 mg; 0.02 mmol; 0.10 eq.) and dioxane-1,4 (2.00 mL). Purification by FCC (DCM/MeOH; gradient). N-[(1-methyl-1H-imidazol-5-yl)methyl]-8-(1-methyl-1H-indol-6-yl)quinoxalin-6-amine (32.80 mg; yield 43%; 96% by HPLC) is obtained as a yellow powder.

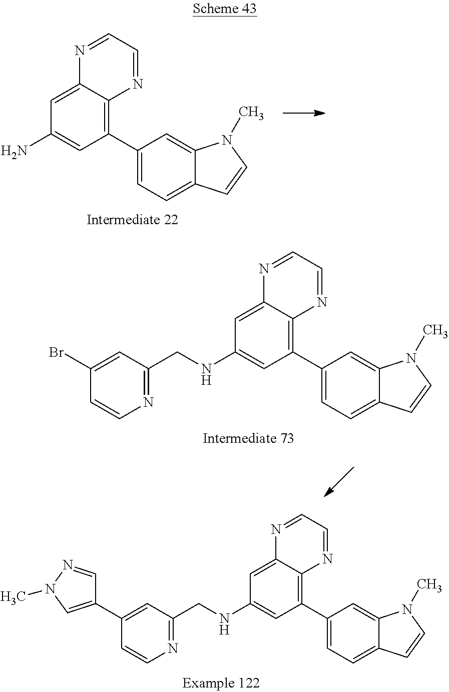 [SMILES: Cn1ccc2ccc(-c3cc(N)cc4nccnc34)cc21 ; Cn1ccc2ccc(-c3cc(NCc4cc(Br)ccn4)cc4nccnc34)cc21 ; Cn1cc(-c2ccnc(CNc3cc(-c4ccc5ccn(C)c5c4)c4nccnc4c3)c2)cn1]

### Intermediate 73

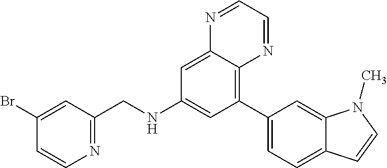 [SMILES: Cn1ccc2ccc(-c3cc(NCc4cc(Br)ccn4)cc4nccnc34)cc21]

The product is prepared according to General Procedure 23, described in Example 63 with 8-(1-methyl-1H-indol-6-yl)-quinoxalin-6-ylamine (Intermediate 22) (100.00 mg; 0.35 mmol; 1.00 eq.), 4-bromopyridine-2-carbaldehyde (65.77 mg; 0.35 mmol; 1.00 eq.), Hantzsch ester (111.96 mg; 0.44 mmol; 1.25 eq.), TMSC (8.98 μl; 0.07 mmol; 0.20 eq.) and DCM anhydrous (4.00 mL). Purification by FCC (DCM/MeOH; gradient). N-[(4-bromopyridin-2-yl)methyl]-8-(1-methyl-1H-indol-6-yl)quinoxalin-6-amine (35.00 mg; yield 21.2%; 95% by HPLC) is obtained as a yellow powder.

### Example 122

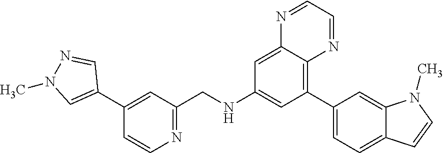 [SMILES: Cn1cc(-c2ccnc(CNc3cc(-c4ccc5ccn(C)c5c4)c4nccnc4c3)c2)cn1]

The product is prepared according to General Procedure 29, described in Example 72 with N-[(4-bromopyridin-2-yl)methyl]-8-(1-methyl-1H-indol-6-yl)quinoxalin-6-amine (Intermediate 73) (25.00 mg; 0.05 mmol; 1.00 eq.), 1-methyl-4-(4,4,5,5-tetramethyl-[1,3,2]dioxaborolan-2-yl)-1H-pyrazole (22.29 mg; 0.11 mmol; 2.00 eq.), KOAc (31.54 mg; 0.32 mmol; 6.00 eq.), Pd(dppf)Cl₂ (9.80 mg; 0.01 mmol; 0.25 eq.), CH₃CN (1.00 mL) and water (0.50 mL). Purification by FCC (column-NH₂ 30 μM; DCM/MeOH; gradient). 8-(1-Methyl-1H-indol-6-yl)-N-{[4-(1-methyl-1H-pyrazol-4-yl)pyridin-2-yl]methyl}quinoxalin-6-amine (23.00 mg; yield 91%; 94% by HPLC) is obtained as a yellow powder.

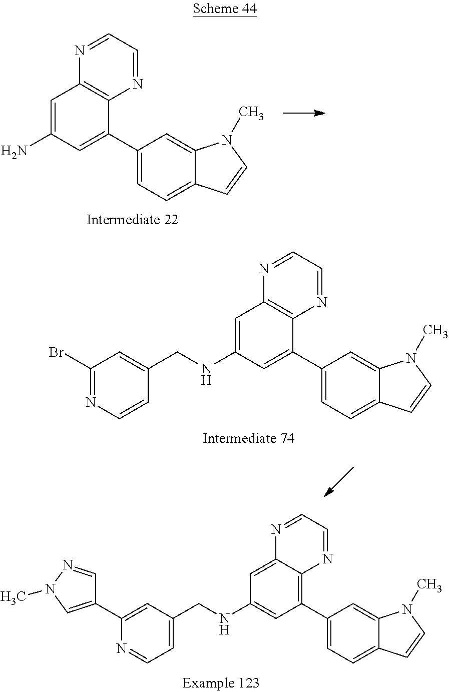 [SMILES: Cn1ccc2ccc(-c3cc(N)cc4nccnc34)cc21 ; Cn1ccc2ccc(-c3cc(NCc4ccnc(Br)c4)cc4nccnc34)cc21 ; Cn1cc(-c2cc(CNc3cc(-c4ccc5ccn(C)c5c4)c4nccnc4c3)ccn2)cn1]

### Intermediate 74

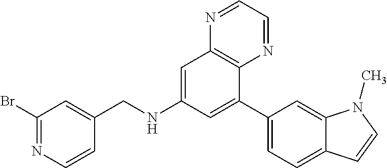 [SMILES: Cn1ccc2ccc(-c3cc(NCc4ccnc(Br)c4)cc4nccnc34)cc21]

The product is prepared according to General Procedure 23, described in Example 63 with 8-(1-methyl-1H-indol-6-yl)-quinoxalin-6-ylamine (Intermediate 22) (100.00 mg; 0.35 mmol; 1.00 eq.), 2-bromopyridine-4-carbaldehyde (65.77 mg; 0.35 mmol; 1.00 eq.), Hantzsch ester (111.96 mg; 0.44 mmol; 1.25 eq.), TMSC (8.98 μl; 0.07 mmol; 0.20 eq.) and DCM anhydrous (4.00 mL). Purification by FCC (DCM/MeOH; gradient). N-[(2-Bromopyridin-4-yl)methyl]-8-(1-methyl-1H-indol-6-yl)quinoxalin-6-amine (65.00 mg; yield 38%; 92% by HPLC) is obtained as a yellow powder.

### Example 123

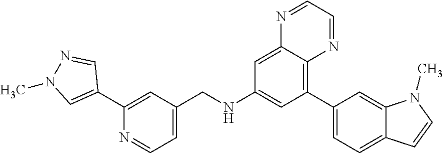 [SMILES: Cn1cc(-c2cc(CNc3cc(-c4ccc5ccn(C)c5c4)c4nccnc4c3)ccn2)cn1]

The product is prepared according to General Procedure 29, described in Example 72 with (2-bromo-pyridin-4-ylmethyl)-[8-(1-methyl-1H-indol-6-yl)-quinoxalin-6-yl]-amine (Intermediate 74) (55.00 mg; 0.11 mmol; 1.00 eq.), 1-methyl-4-(4,4,5,5-tetramethyl-[1,3,2]dioxaborolan-2-yl)-1H-pyrazole (47.54 mg; 0.23 mmol; 2.00 eq.), KOAc (67.28 mg; 0.69 mmol; 6.00 eq.), Pd(dppf)Cl₂ (20.90 mg; 0.03 mmol; 0.25 eq.), CH₃CN (2.00 mL) and water (1.00 mL). Purification by FCC (column-NH₂ 30 μM chromatography; MeOH/DCM; gradient). 8-(1-methyl-1H-indol-6-yl)-N-{[2-(1-methyl-1H-pyrazol-4-yl)236yridine-4-yl]methyl}quinoxalin-6-amine (35.00 mg; yield 66%; 96% by HPLC) as a yellow powder.

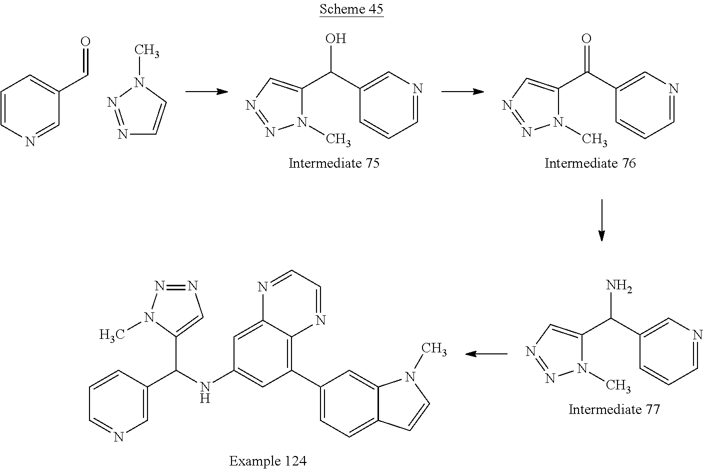

### Intermediate 75—General Procedure 49

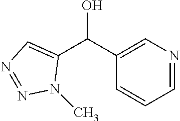

To a 2-necked flask containing 1-methyl-1H-[1,2,3]triazole (162.91 mg; 1.96 mmol; 1.05 eq.) is added anhydrous THF (4.00 mL) and the solution is cooled between −40 to −20° C. To this colorless solution n-BuLi in hexane 1.6M (1.23 ml; 1.96 mmol; 1.05 eq.) is added dropwise. After stirring at 0° C. for 1 hour, a solution of pyridine-3-carbaldehyde (175.28 μL; 1.87 mmol; 1.00 eq.) in anhydrous THF (3.00 mL) is added and the reaction mixture is stirred for 3 h. After this time, RM is quenched by pouring into a saturated solution of NH₄Cl. Aqueous phase is extracted 3 times with n-BuOH. Organic layer is dried over Na₂SO₄, filtered and concentrated in vacuo to obtain: (3-methyl-3H-[1,2,3]triazol-4-yl)-pyridin-3-yl-methanol (243.00 mg; yield 66%; 99% by UPLC) is obtained as a beige oil.

### Intermediate 76—General Procedure 50

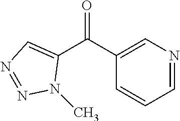

A flask containing Dess-Martin Reagent (858.34 mg; 2.02 mmol; 1.60 eq.) in DCM is cooled to 0° C. and then a solution of (3-methyl-3H-[1,2,3]triazol-4-yl)-pyridin-3-yl-methanol (Intermediate 75) (243.00 mg; 1.26 mmol; 1.00 eq.) in DCM is added. After 5 min ice bath is removed and the mixture is allowed to stir at RT for 45 minutes. RM is quenched with a saturated NaHCO₃ solution and 1N NaOH solution. Aqueous layer is extracted with DCM. Crude product is purified by FCC (Hexane/EtOAc; gradient). (3-Methyl-3H-[1,2,3]triazol-4-yl)-pyridin-3-yl-methanone (160.00 mg; yield 64%; 95% by UPLC) is obtained as a beige solid.

### Intermediate 77

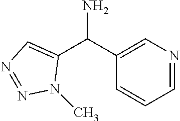

The product is prepared according to General Procedure 14, described for Intermediate 12 with (3-methyl-3H-[1,2,3]triazol-4-yl)-pyridin-3-yl-methanone (Intermediate 76) (160.00 mg; 0.81 mmol; 1.00 eq.), 7M NH₃ in MeOH (3.50 mL), TTIP (0.48 mL; 1.62 mmol; 2.00 eq.) and NaBH₄ (122.23 mg; 3.23 mmol; 4.00 eq.). Extraction with EtOAc and n-BuOH. C-(3-Methyl-3H-[1,2,3]triazol-4-yl)-C-pyridin-3-ylmethylamine (130.00 mg; yield 38%; 45% by UPLC) is directly used in the next step without further purification.

### Example 124

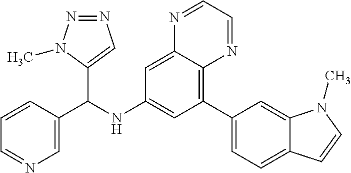 [SMILES: Cn1nncc1C(Nc1cc(-c2ccc3ccn(C)c3c2)c2nccnc2c1)c1cccnc1]

The product is prepared according to General Procedure 2, described in Example 1 with 7-chloro-5-(1-methyl-1H-indol-6-yl)-quinoxaline (Intermediate 4) (40.00 mg; 0.13 mmol; 1.00 eq.), C-(3-methyl-3H-[1,2,3]triazol-4-yl)-C-pyridin-3-yl-methylamine (Intermediate 77) (84.17 mg; 0.20 mmol; 1.50 eq.), NaOtBu (44.89 mg; 0.47 mmol; 3.50 eq.), BINAP (16.62 mg; 0.03 mmol; 0.20 eq.), Pd₂(dba)₃ (12.22 mg; 0.01 mmol; 0.10 eq.) and toluene (3.00 mL). Purification by FCC (hexane/EtOAc; gradient). N-[(1-methyl-1H-1,2,3-triazol-5-yl)(pyridin-3-yl)methyl]-8-(1-methyl-1H-indol-6-yl)quinoxalin-6-amine (28.00 mg; yield 43%; 92% by HPLC) as an orange solid.

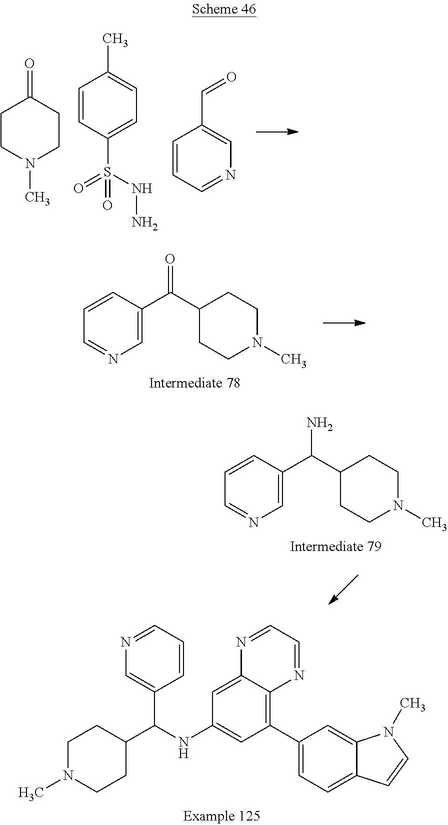

### Intermediate 78

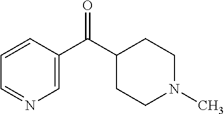

The product is prepared according to General Procedure 15, described for Intermediate 13 with 1-methylpiperidin-4-one (300.00 mg; 2.65 mmol; 1.00 eq.), 4-methylbenzenesulfonohydrazide (493.73 mg; 2.65 mmol; 1.00 eq.), Cs₂CO₃ (647.85 mg; 1.99 mmol; 0.75 eq.), pyridine-3-carbaldehyde (283.97 mg; 2.65 mmol; 1.00 eq.), MeOH (3.00 mL) and 1,4-dioxane (3.00 mL). Purification by FCC (DCM/MeOH; gradient; silica gel, deactivated with TEA). (1-Methyl-piperidin-4-yl)-pyridin-3-yl-methanone (124.00 mg; yield 14%; 62% by UPLC) is obtained as a yellow oil.

### Intermediate 79

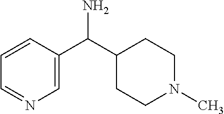

The product is prepared according to General Procedure 14, described for Intermediate 12 with (1-methylpiperidin-4-yl)-pyridin-3-yl-methanone (Intermediate 78) (124.00 mg; 0.38 mmol; 1.00 eq.), TTIP (0.23 ml; 0.76 mmol; 2.00 eq.), 7M NH₃ in MeOH (2.00 mL) and NaBH₄ (57.88 mg; 1.53 mmol; 4.00 eq.). Extraction with EtOAc and n-BuOH. C-(1-Methylpiperidin-4-yl)-C-pyridin-3-yl-methylamine (98.00 mg; yield 77%, 62% by UPLC) is directly used in the next step without further purification.

### Example 125

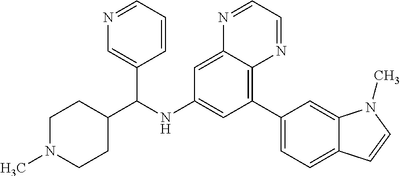 [SMILES: CN1CCC(C(Nc2cc(-c3ccc4ccn(C)c4c3)c3nccnc3c2)c2cccnc2)CC1]

The product is prepared according to General Procedure 2, described in Example 1 with 7-chloro-5-(1-methyl-1H-indol-6-yl)-quinoxaline (Intermediate 4) (45.00 mg; 0.15 mmol; 1.00 eq.), C-(1-methylpiperidin-4-yl)-C-pyridin-3-yl-methylamine (Intermediate 79) (74.57 mg; 0.23 mmol; 1.50 eq.), NaOtBu (50.50 mg; 0.53 mmol; 3.50 eq.), BINAP (18.70 mg; 0.03 mmol; 0.20 eq.), Pd₂(dba)₃ (13.75 mg; 0.02 mmol; 0.10 eq.) and toluene (3.00 mL). Purification by FCC (column-NH₂ 30 μM; DCM/MeOH; gradient). 8-(1-methyl-1H-indol-6-yl)-N-[(1-methylpiperidin-4-yl)(pyridin-3-yl)methyl]quinoxalin-6-amine (32.50 mg; yield 45%; 96% by HPLC) is obtained as a yellow amorphous powder.

### Example 126

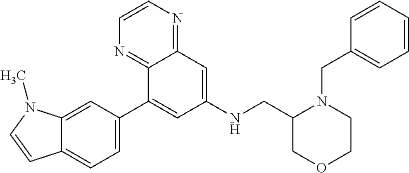 [SMILES: Cn1ccc2ccc(-c3cc(NCC4COCCN4Cc4ccccc4)cc4nccnc34)cc21]

The product is prepared according to General Procedure 2, described in Example 1 with 7-chloro-5-(1-methyl-1H-indol-6-yl)-quinoxaline (Intermediate 4) (150.00 mg; 0.51 mmol; 1.00 eq.), C-(4-benzylmorpholin-3-yl)-methylamine (0.15 ml; 0.77 mmol; 1.50 eq.), NaOtBu (98.15 mg; 1.02 mmol; 2.00 eq.), BINAP (64.89 mg; 0.10 mmol; 0.20 eq.), Pd₂(dba)₃ (46.76 mg; 0.05 mmol; 0.10 eq.) and toluene anhydrous (5.00 mL). Purification by FCC (DCM/MeOH; gradient) and repurification by preparative HPLC. N-[(4-Benzylmorpholin-3-yl)methyl]-8-(1-methyl-1H-indol-6-yl)quinoxalin-6-amine (210.00 mg; yield 86.0%; 96.9% by HPLC) is obtained as a yellow powder.

### Example 127

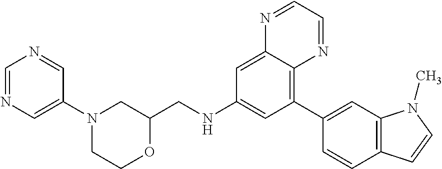 [SMILES: Cn1ccc2ccc(-c3cc(NCC4CN(c5cncnc5)CCO4)cc4nccnc34)cc21]

The product is prepared according to General Procedure 30, described in Example 74 with [8-(1-methyl-1H-indol-6-yl)-quinoxalin-6-yl]-morpholin-2-ylmethyl-amine (Example 32) (20.00 mg; 0.05 mmol; 1.00 eq.), 5-bromopyrimidine (7.83 mg; 0.05 mmol; 1.00 eq.), NaOtBu (7.09 mg; 0.07 mmol; 1.50 eq.), Pd₂(dba)₃ (1.80 mg; 0.00 mmol; 0.04 eq.), Xantphos (3.42 mg; 0.01 mmol; 0.12 eq.) and toluene (1.5 mL). Purification by FFC (PF-ALN/7G; hexane/EtOAc; gradient next EtOAc/MeOH; gradient). 8-(1-methyl-1H-indol-6-yl)-N-{[4-(pyrimidin-5-yl)morpholin-2-yl]methyl}quinoxalin-6-amine (12.00 mg; yield 49.6%; 92% by HPLC) is obtained as a yellow solid.

### Example 128

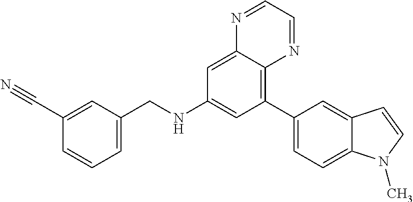 [SMILES: Cn1ccc2cc(-c3cc(NCc4cccc(C#N)c4)cc4nccnc34)ccc21]

The product is prepared according to General Procedure 6 described for the Intermediate 6 with 7-chloro-5-(1-methyl-1H-indol-5-yl)-quinoxaline (Intermediate 29) (200.00 mg; 0.65 mmol; 1.00 eq.), 3-aminomethylbenzonitrile (0.12 mL; 0.97 mmol; 1.50 eq.), Cs₂CO₃ (638.62 mg; 1.94 mmol; 3.00 eq.), BINAP (20.55 mg; 0.03 mmol; 0.05 eq.) and Pd(OAc)₂ (7.64 mg; 0.03 mmol; 0.05 eq.) and 1,4-dioxane (10.00 mL). Purification by FCC (DCM/MeOH; gradient). 3-({[8-(1-Methyl-1H-indol-5-yl)quinoxalin-6-yl]amino}methyl)benzonitrile (28.90 mg; 0.07 mmol; yield 10.9%; 95% by HPLC) is obtained as a yellow powder.

### Example 129

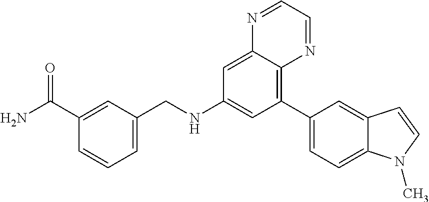 [SMILES: Cn1ccc2cc(-c3cc(NCc4cccc(C(N)=O)c4)cc4nccnc34)ccc21]

The product is prepared according to General Procedure 7 described for Example 35 with 3-({[8-(1-methyl-1H-indol-5-yl)quinoxalin-6-yl]amino}methyl)benzonitrile (Example 128), tert-butanol (4.00 mL) and potassium hydroxide (21.61 mg; 0.39 mmol; 3.00 eq.). Purification by FCC (MeOH/DCM, gradient). 3-({[8-(1-Methyl-1H-indol-5-yl)quinoxalin-6-yl]amino}methyl)benzamide benzamide (30.50 mg; 0.07 mmol; yield 57.3%; 98.2 by HPLC) is obtained as yellow foam.

### Example 130

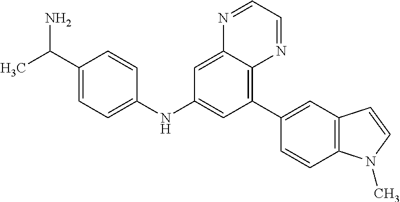 [SMILES: CC(N)c1ccc(Nc2cc(-c3ccc4c(ccn4C)c3)c3nccnc3c2)cc1]

The product is prepared according to General Procedure 2, described in Example 1 with 7-chloro-5-(1-methyl-1H-indol-5-yl)-quinoxaline (Intermediate 29) (70.00 mg; 0.23 mmol; 1.00 eq.), 4-(1-aminoethyl)aniline (74.00 mg; 0.54 mmol; 2.40 eq.), NaOtBu (65.27 mg; 0.68 mmol; 3.00 eq.), BINAP (28.19 mg; 0.05 mmol; 0.20 eq.), Pd₂(dba)₃ (52.55 mg; 0.02 mmol; 0.10 eq.) and toluene (4.00 mL). Purification by FCC (MeOH/DCM, gradient). Repurification by preparative HPLC (ACN/0.1% aq ammonia, gradient). N-[4-(1-aminoethyl)phenyl]-8-(1-methyl-1H-indol-5-yl)quinoxalin-6-amine (47.30 mg; 0.12 mmol; yield 51.5%; 96.6% by HPLC) is obtained as yellow amorphous powder.

### Example 131

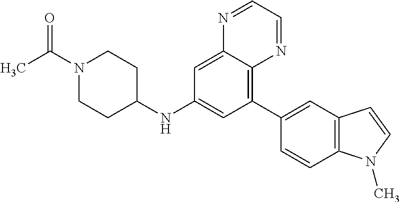 [SMILES: CC(=O)N1CCC(Nc2cc(-c3ccc4c(ccn4C)c3)c3nccnc3c2)CC1]

The product is prepared according to General Procedure 2, described in Example 1 with 7-chloro-5-(1-methyl-1H-indol-5-yl)-quinoxaline (Intermediate 29) 50.00 mg; 0.17 mmol; 1.00 eq.), 1-(4-aminopiperidin-1-yl)ethan-1-one (48.41 mg; 0.34 mmol; 2.00 eq.), NaOtBu (49.07 mg; 0.51 mmol; 3.00 eq.), BINAP (21.20 mg; 0.03 mmol; 0.20 eq.), Pd₂(dba)₃ (15.59 mg; 0.02 mmol; 0.10 eq.), toluene (2.00 mL). Reaction is carried out in a well-sealed tube with silicone PTFE coated cap at 120° C. for 24 h. Purification by FCC (DCM/MeOH; gradient). 1-(4-{[8-(1-Methyl-1H-indol-5-yl)quinoxalin-6-yl]amino}piperidin-1-yl)ethan-1-one (48.60 mg; 0.12 mmol; yield 68.6%; 96.2 by HPLC) is obtained as yellow powder.

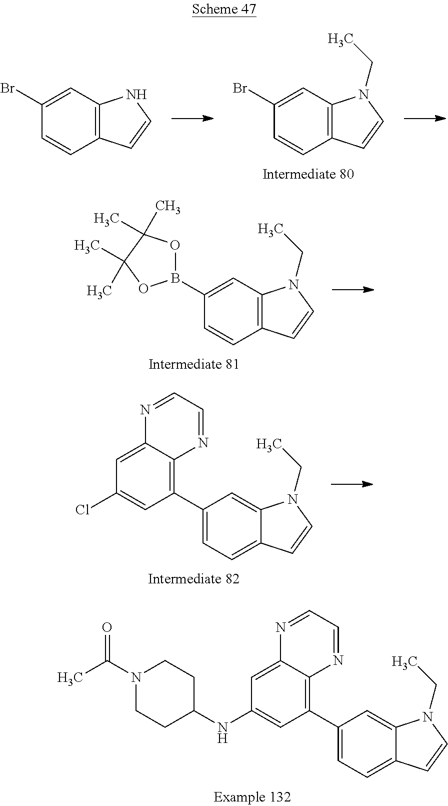 [SMILES: Brc1ccc2cc[nH]c2c1 ; CCn1ccc2ccc(Br)cc21 ; CCn1ccc2ccc(B3OC(C)(C)C(C)(C)O3)cc21 ; CCn1ccc2ccc(-c3cc(Cl)cc4nccnc34)cc21 ; CCn1ccc2ccc(-c3cc(NC4CCN(C(C)=O)CC4)cc4nccnc34)cc21]

### Intermediate 80

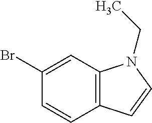

Product is prepared according to procedure described in literature (Fraile, J. M.; Le Jeune, K.; Mayoral, J. A.; Ravasio, N.; Zaccheria, F.; Org. Biomol. Chem. 2013, v:11, pp: 4327-4332): A solution of 6-bromo-1H-indole (0.50 g; 2.55 mmol; 1.00 eq.) in dry THF (10.00 ml), is cooled to 0-5° C. then NaH (60% immersion in mineral oil) (0.20 g; 5.10 mmol; 2.00 eq.) is added in small portions over 10 min. RM is stirred for 1h then Iodoethane (0.27 ml; 3.32 mmol; 1.30 eq.) is added dropwise. The reaction mixture is stirred for next 30 min at 0° C. and 1 h at rt. After mentioned time RM is poured onto ice and extracted with diethyl ether. Organic layers is washed with brine and dried over Na₂SO₄. Solvent is evaporated in vacuo to provide desired product 6-Bromo-1-ethyl-1H-indole (0.594 g; 2.35 mmol; yield 92.3%; 89% by UPLC) is obtained as brown oil.

### Intermediate 81—General Procedure 51 for Miyaura Coupling Borylation

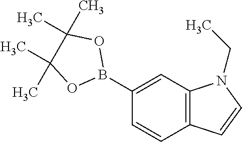 [SMILES: CCn1ccc2ccc(B3OC(C)(C)C(C)(C)O3)cc21]

The sealed tube is charged with 6-bromo-1-ethyl-1H-indole (Intermediate 80) (0.59 g; 2.35 mmol; 1.00 eq.), 4,4,5,5-tetramethyl-2-(tetramethyl-1,3,2-dioxaborolan-2-yl)-1,3,2-dioxaborolane (0.78 g; 3.06 mmol; 1.30 eq.), 1,4-Dioxane (5.00 ml) and KOAc (0.46 g; 4.71 mmol; 2.00 eq.). Resulted slurry is flashed with argon and then Pd(dppf)Cl₂ (172 mg; 0.02 mmol; 0.1 eq.) is added under argon and tube is capped. RM is heated in an oil bath preheated to 100° C. under stirring for 18 h. After this time, the mixture is diluted with EtOAc, filtered through a Celite® pad. The filtrate is collected and evaporated. Crude product is purified by FCC (hexane/EtOAc; gradient). 1-Ethyl-6-(tetramethyl-1,3,2-dioxaborolan-2-yl)-1H-indole (0.258 g; 0.84 mmol; Yield 35.5%; 88% by UPLC) is obtained as light brown oil.

### Intermediate 82

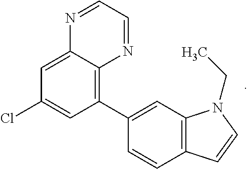 [SMILES: CCn1ccc2ccc(-c3cc(Cl)cc4nccnc34)cc21]

The product is prepared according to General Procedure 1, described for Intermediate 4 with 5-bromo-7-chloroquinoxaline (Intermediate 2) (0.26 g; 1.07 mmol; 1.00 eq.), 1-Ethyl-6-(tetramethyl-1,3,2-dioxaborolan-2-yl)-1H-indole (Intermediate 81) (0.25 g; 0.80 mmol; 0.75 eq.), DIPEA (0.37 ml; 2.14 mmol; 2.00 eq.), Pd(dppf)Cl₂ ((78 mg; 0.11 mmol; 0.10 eq.), water (6 ml) and [1,4]-dioxane (12.00 ml). Reaction is carried out for 2.5 h at 85° C. Purification by FCC (Hexane/EtOAc, gradient). 7-Chloro-5-(1-ethyl-1H-indol-6-yl)quinoxaline (0.19 g; 0.63 mmol; yield 59.0%; 100% by UPLC) is obtained as yellow solid.

### Example 132

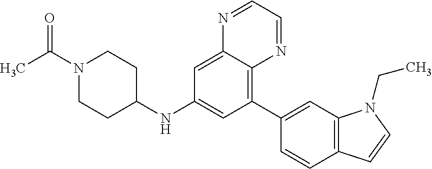 [SMILES: CCn1ccc2ccc(-c3cc(NC4CCN(C(C)=O)CC4)cc4nccnc34)cc21]

Product is prepared according to General Procedure 2, described in Example 1 with 7-chloro-5-(1-ethyl-1H-indol-6-yl)quinoxaline (Intermediate 80) (50.00 mg; 0.17 mmol; 1.00 eq.), 1-(4-aminopiperidin-1-yl)ethan-1-one (48.41 mg; 0.34 mmol; 2.00 eq.), NaOtBu (62 mg; 0.65 mmol; 4.00 eq.), BINAP (21.20 mg; 0.03 mmol; 0.20 eq.), Pd₂(dba)₃ (16 mg; 0.02 mmol; 0.10 eq.) and Toluene (2.00 ml). Reaction is carried out for 18 h at 120° C. Purification by FCC (MeOH/DCM, gradient). 1-(4-{[8-(1-Ethyl-1H-indol-6-yl)quinoxalin-6-yl]amino}piperidin-1-yl)ethan-1-one ethanone (48.60 mg; 0.12 mmol; 68.6%; 91.5% by HPLC) is obtained as yellow powder.

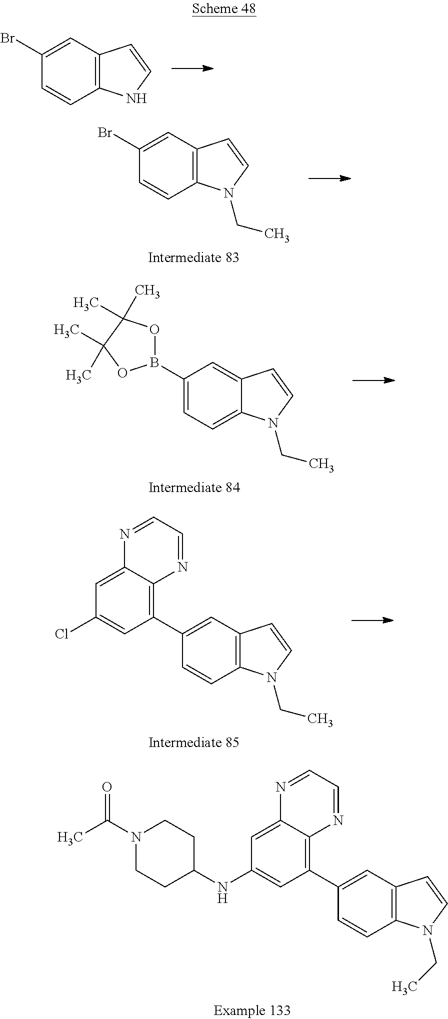

### Intermediate 83

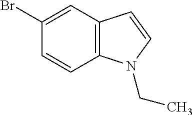

Product is prepared according to procedure described in literature (Fraile, J. M.; Le Jeune, K.; Mayoral, J. A.; Ravasio, N.; Zaccheria, F.; Org. Biomol. Chem. 2013, v:11, pp: 4327-4332). A solution of 5-bromo-1H-indole (0.50 g; 2.55 mmol; 1.00 eq.) in dry THF (10.00 ml), is cooled to 0-5° C. then NaH (60% immersion in mineral oil) (0.20 g; 5.10 mmol; 2.00 eq.) is added in small portions over 10 min. RM is stirred for 1h then Iodoethane (0.27 ml; 3.32 mmol; 1.30 eq.) is added dropwise. Reaction mixture is stirred for next 30 min at 0° C. and 1h at rt. After mentioned time RM is poured onto ice and extracted with diethyl ether. Organic layers is washed with brine and dried over Na₂SO₄. Solvent is evaporated in vacuo to provide desired product 6-Bromo-1-ethyl-1H-indole (0.612 g; 2.51 mmol; yield 98.4%; 92% by UPLC) is obtained as light brown oil.

### Intermediate 84

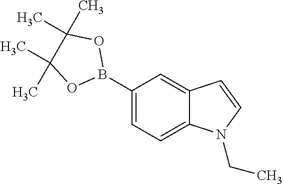 [SMILES: CCn1ccc2cc(B3OC(C)(C)C(C)(C)O3)ccc21]

Product is prepared according to General Procedure 51 for Miyaura coupling borylation, described for Intermediate 81 with with 5-Bromo-1-ethyl-1H-indole (Intermediate 83) (0.59 g; 2.35 mmol; 1.00 eq.), 4,4,5,5-tetramethyl-2-(tetramethyl-1,3,2-dioxaborolan-2-yl)-1,3,2-dioxaborolane (0.78 g; 3.06 mmol; 1.30 eq.), KOAc (0.46 g; 4.71 mmol; 2.00 eq.), Pd(dppf)Cl₂ (172 mg; 0.02 mmol; 0.1 eq.) and [1,4]-dioxane (5.00 ml). Reaction is carried out for 18 h at 100° C. Purification by FCC (hexane/EtOAc; gradient). 1-Ethyl-6-(tetramethyl-1,3,2-dioxaborolan-2-yl)-1H-indole (0.54 g; 1.64 mmol; Yield 68%; 82% by UPLC) is obtained as colorless oil.

### Intermediate 85

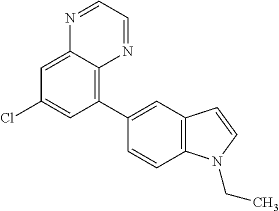 [SMILES: CCn1ccc2cc(-c3cc(Cl)cc4nccnc34)ccc21]

Product is prepared according to General Procedure 1, described for Intermediate 4 with 5-Bromo-7-chloroquinoxaline (Intermediate 2) (0.37 g; 1.52 mmol; 1.00 eq.), 1-Ethyl-6-(tetramethyl-1,3,2-dioxaborolan-2-yl)-1H-indole (Intermediate 84) (0.55 g; 1.67 mmol; 1.10 eq.), DIPEA (0.53 ml; 3.04 mmol; 2.00 eq.), Pd(dppf)Cl₂ (111 mg; 0.15 mmol; 0.10 eq.), water (2.5 ml) and [1,4]-dioxane (5.0 mL). Reaction is carried out for 3 h at 85° C. Purification by FCC (Hexane/EtOAc, gradient). 7-Chloro-5-(1-ethyl-1H-indol-6-yl)quinoxaline (0.304 g; 0.99 mmol; yield 65.0%; 82% by UPLC) is obtained as beige solid.

### Example 133

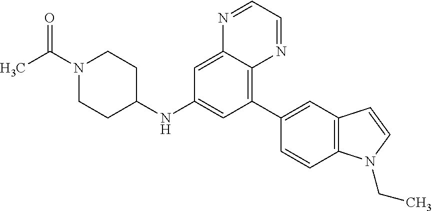 [SMILES: CCn1ccc2cc(-c3cc(NC4CCN(C(C)=O)CC4)cc4nccnc34)ccc21]

Product is prepared according to General Procedure 2, described in Example 1 with 7-Chloro-5-(1-ethyl-1H-indol-5-yl)quinoxaline (Intermediate 85) (50.00 mg; 0.16 mmol; 1.00 eq.), 1-(4-aminopiperidin-1-yl)ethan-1-one hydrochloride (58 mg; 0.32 mmol; 2.00 eq.), NaOtBu (63 mg; 0.65 mmol; 4.00 eq.), BINAP (20 mg; 0.03 mmol; 0.20 eq.), Pd₂(dba)₃ (15 mg; 0.02 mmol; 0.10 eq.) and Toluene (2.00 mL). Reaction is carried out for 18 h at 120° C. Purification by FCC (MeOH/DCM, gradient). 1-(4-{[8-(1-Ethyl-1H-indol-6-yl)quinoxalin-6-yl]amino}piperidin-1-yl)ethan-1-one ethanone (33.20 mg; 0.08 mmol; yield 49.2%; 99.5% by HPLC) is obtained as yellow-greenish solid.

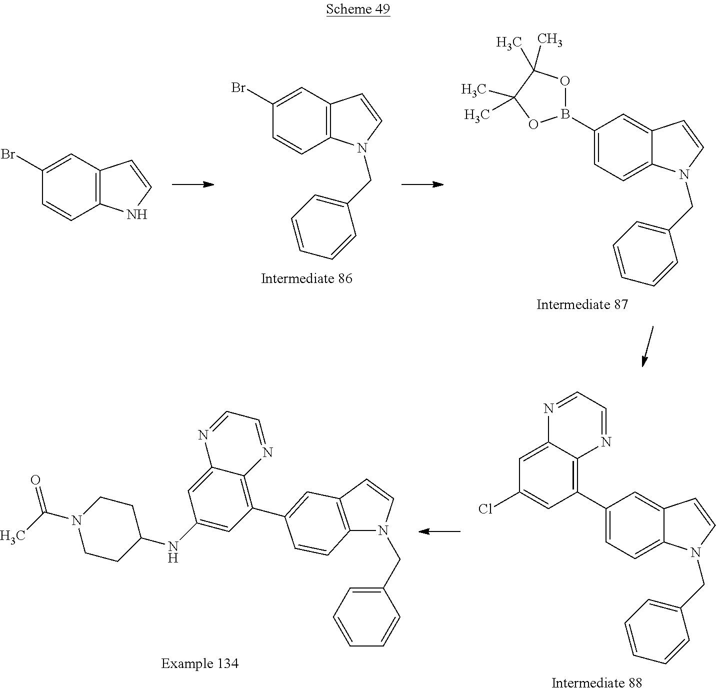 [SMILES: Brc1ccc2[nH]ccc2c1 ; Brc1ccc2c(ccn2Cc2ccccc2)c1 ; CC1(C)OB(c2ccc3c(ccn3Cc3ccccc3)c2)OC1(C)C ; CC(=O)N1CCC(Nc2cc(-c3ccc4c(ccn4Cc4ccccc4)c3)c3nccnc3c2)CC1 ; Clc1cc(-c2ccc3c(ccn3Cc3ccccc3)c2)c2nccnc2c1]

### Intermediate 86

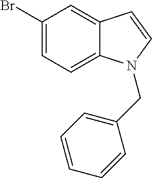 [SMILES: Brc1ccc2c(ccn2Cc2ccccc2)c1]

The product is prepared similar to procedure described in US2003125371 A1. To a solution of 5-bromo-1H-indole (1.00 g; 5.10 mmol; 1.00 eq.) in DMF (10.00 ml) sodium hydride (60% in mineral oil) (0.44 g; 11.00 mmol; 2.16 eq.) is added and the resulting mixture is stirred for 30 minutes at rt. Then the resulting mixture is placed in an ice bath and (bromomethyl)benzene (1.22 mL; 10.17 mmol; 1.99 eq.) is added. RM is stirred at rt for 1 h. Then it is poured on water. The resulting mixture is acidified using 2M HCl and then it is extracted with EtOAc. The organic layer is washed with water, brine, dried over anhydrous Na₂SO₄ and filtered. The filtrate is evaporated under reduced pressure and the residue is purified by FCC (hexane/DCM; gradient) to give 1-benzyl-5-bromo-1H-indole (1.25 g; yield 67.7%; 79.3% by UPLC) as a colorless crystallizing oil.

### Intermediate 87

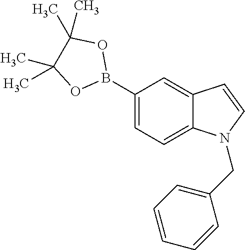 [SMILES: CC1(C)OB(c2ccc3c(ccn3Cc3ccccc3)c2)OC1(C)C]

Product is prepared according to General Procedure 51 for Miyaura coupling borylation, described for Intermediate 81 with 1-benzyl-5-bromo-1H-indole (Intermediate 86) (1.247 g; 3.46 mmol; 1.00 eq.), bis(pinacolato)diboron (1.141 g; 4.49 mmol; 1.30 eq.), Pd(dppf)Cl₂—CH₂Cl₂ (25 mg; 0.03 mmol; 0.01 eq.), 1,4-dioxane (5.000 mL) and KOAc (0.678 g; 6.91 mmol; 2.00 eq.). Reaction is carried out overnight at 100° C. After filtration through Celite® diluted RM is partitioned between EtOAc and water. The organic phase is dried and evaporated. Purification by FCC (hexane/EtOAc; gradient) to give 1-benzyl-5-(tetramethyl-1,3,2-dioxaborolan-2-yl)-1H-indole (925 mg; yield 59.0%; 73.4% by UPLC) as a colorless oil.

### Intermediate 88

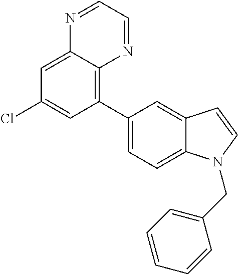 [SMILES: Clc1cc(-c2ccc3c(ccn3Cc3ccccc3)c2)c2nccnc2c1]

The product is prepared according to General Procedure 1, described for Intermediate 4 with 5-bromo-7-chloroquinoxaline (Intermediate 2) (679 mg; 2.77 mmol; 1.36 eq.), 1-benzyl-5-(tetramethyl-1,3,2-dioxaborolan-2-yl)-1H-indole (Intermediate 87) (925 mg; 2.04 mmol; 1.00 eq.), DIPEA (0.967 ml; 5.55 mmol; 2.72 eq.), Pd(dppf)Cl₂ (203 mg; 0.28 mmol; 0.14 eq.), 1,4-dioxane (2.500 ml) and water (2.500 ml). Reaction is carried out for 2.5 h at 85° C. Then RM is diluted with AcOEt and filtered through Celite®. The filtrate is concentrated and the residue is purified by FCC (hexane/AcOEt; gradient) to give 5-(1-benzyl-1H-indol-5-yl)-7-chloroquinoxaline (803.70 mg; yield 88.1%; 82.6% by UPLC) as a yellow solid.

### Example 134

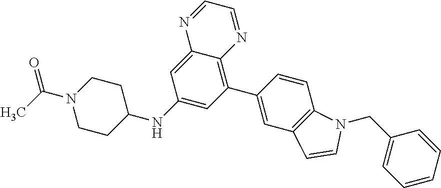 [SMILES: CC(=O)N1CCC(Nc2cc(-c3ccc4c(ccn4Cc4ccccc4)c3)c3nccnc3c2)CC1]

The product is prepared according to General Procedure 2, described in Example 1 with 1-(4-aminopiperidin-1-yl)ethan-1-one hydrochloride (55.9 mg; 0.31 mmol; 2.00 eq.), Pd₂(dba)₃ (14.3 mg; 0.02 mmol; 0.10 eq.), NaOtBu (60.1 mg; 0.63 mmol; 4.00 eq.), BINAP (19.5 mg; 0.03 mmol; 0.20 eq.), 5-(1-benzyl-1H-indol-5-yl)-7-chloroquinoxaline (Intermediate 88) (70 mg; 0.16 mmol; 1.00 eq.) and toluene (2.00 mL). Reaction is carried out overnight at 120° C. RM is diluted with AcOEt and filtered thorough Celite®. The filtrate is evaporated and the residue is purified by FCC (DCM/MeOH; gradient) to give 1-(4-{[8-(1-benzyl-1H-indol-5-yl)quinoxalin-6-yl]amino}piperidin-1-yl)ethan-1-one (27.6 mg; yield 36.3%; 97.90% by HPLC) as a green yellow powder.

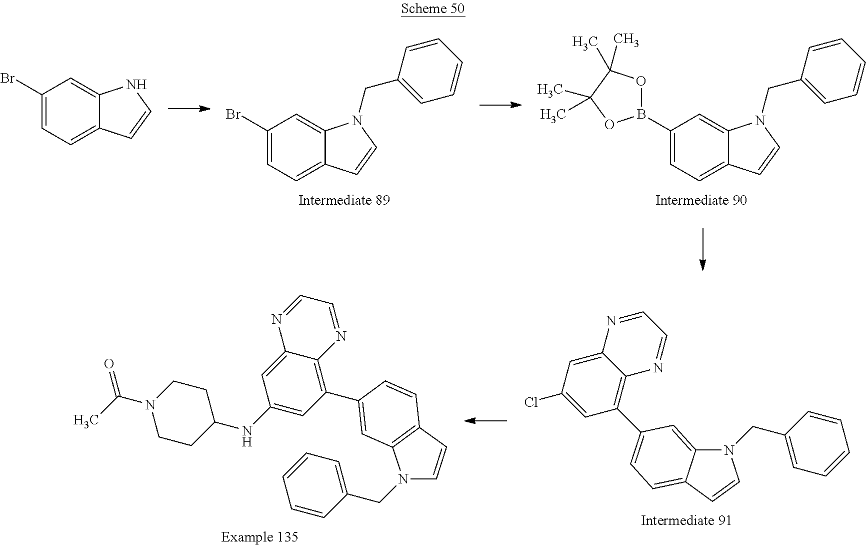 [SMILES: Brc1ccc2cc[nH]c2c1 ; Brc1ccc2ccn(Cc3ccccc3)c2c1 ; CC1(C)OB(c2ccc3ccn(Cc4ccccc4)c3c2)OC1(C)C ; CC(=O)N1CCC(Nc2cc(-c3ccc4ccn(Cc5ccccc5)c4c3)c3nccnc3c2)CC1 ; Clc1cc(-c2ccc3ccn(Cc4ccccc4)c3c2)c2nccnc2c1]

### Intermediate 89

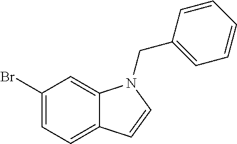 [SMILES: Brc1ccc2ccn(Cc3ccccc3)c2c1]

The product is prepared similar to procedure described in US 2003/125371 A1. To a solution of 6-bromo-1H-indole (1 g; 5.10 mmol; 1 eq.) in DMF (10 ml) NaH 60% (in mineral oil) (0.44 g; 11.00 mmol; 2.16 eq.) is added and the resulting mixture is stirred for 30 minutes at RT. Then the mixture is placed in an ice bath and (bromomethyl)benzene (1.22 ml; 10.17 mmol; 1.99 eq.) is added. RM is stirred for 1 h at rt and then it is poured on water. The resulting mixture is acidified using 2M HCl and then it is extracted with EtOAc. The organic is washed with water, brine, dried over anhydrous Na₂SO₄ and filtered. The filtrate is evaporated under reduced pressure and the residue is purified by FCC (hexane/DCM; gradient) to give 1-benzyl-6-bromo-1H-indole (1.02 g; yield 50.9%; 72.8% by UPLC) as a white solid.

### Intermediate 90

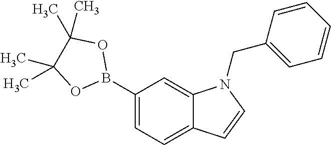 [SMILES: CC1(C)OB(c2ccc3ccn(Cc4ccccc4)c3c2)OC1(C)C]

Product is prepared according to General Procedure 51 for Miyaura coupling borylation, described for Intermediate 81 with 1-benzyl-6-bromo-1H-indole (Intermediate 89) (1.02 g; 2.59 mmol; 1 eq.), bis(pinacolato)diboron (0.857 g; 3.37 mmol; 1.3 eq.), KOAc (0.509 g; 5.19 mmol; 2 eq.), Pd(dppf)Cl₂—CH₂Cl₂ (25 mg; 0.03 mmol; 0.01 eq.) and 1,4-dioxane (5 ml). Reaction is carried out overnight at 100° C. After filtration through Celite® diluted RM is partitioned between EtOAc and water. The organic phase is dried and evaporated. Purification by FCC (hexane/EtOAc; gradient) to give 1-benzyl-6-(tetramethyl-1,3,2-dioxaborolan-2-yl)-1H-indole (621 mg; yield 25.9%; 36.0% by UPLC) as a colorless oil.

### Intermediate 91

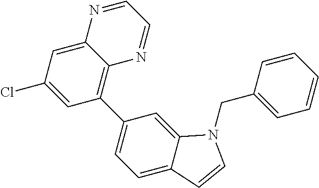 [SMILES: Clc1cc(-c2ccc3ccn(Cc4ccccc4)c3c2)c2nccnc2c1]

The product is prepared according to general Procedure 1, described for Intermediate 4 with 5-bromo-7-chloroquinoxaline (Intermediate 2) (399 mg; 1.63 mmol; 0.87 eq.), 1-benzyl-6-(tetramethyl-1,3,2-dioxaborolan-2-yl)-1H-indole (Intermediate 90) (621 mg; 1.86 mmol; 1 eq.), Pd(dppf)Cl₂ (120 mg; 0.16 mmol; 0.09 eq.), DIPEA (0.571 ml; 3.28 mmol; 1.76 eq.), 1,4-dioxane (4 ml) and water (4 ml). Reaction is carried out for 2.5 h at 85° C. Then RM is diluted with EtOAc and filtered through Celite®. The filtrate is concentrated and the residue is purified by FCC (hexane/DCM; gradient) to give 5-(1-benzyl-1H-indol-6-yl)-7-chloroquinoxaline (332.9 mg; yield 46%; 95.3% by UPLC) as a yellow solid.

### Example 135

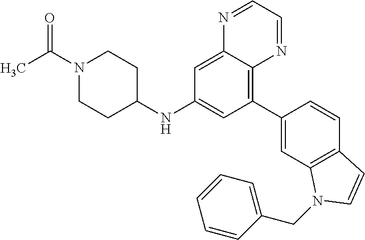 [SMILES: CC(=O)N1CCC(Nc2cc(-c3ccc4ccn(Cc5ccccc5)c4c3)c3nccnc3c2)CC1]

The product is prepared according to General Procedure 2, described in Example 1 with 5-(1-benzyl-1H-indol-6-yl)-7-chloroquinoxaline (Intermediate 91) (70 mg; 0.18 mmol; 1.00 eq.), 1-(4-aminopiperidin-1-yl)ethan-1-one hydrochloride (64.45 mg; 0.36 mmol; 2.00 eq.), NaOtBu (69.34 mg; 0.72 mmol; 4.00 eq.), Pd₂(dba)₃ (16.52 mg; 0.02 mmol; 0.10 eq.), BINAP (22.46 mg; 0.04 mmol; 0.20 eq.) and toluene (2 ml). Reaction is carried out overnight at 120° C. Then RM is diluted with AcOEt and filtered thorough Celite®. The filtrate is evaporated and the residue is purified by FCC (DCM/MeOH; gradient) to give 1-(4-{[8-(1-benzyl-1H-indol-6-yl)quinoxalin-6-yl]amino}piperidin-1-yl)ethan-1-one (42.2 mg; yield 48.1%; 97.8% by HPLC) as a green yellow powder.

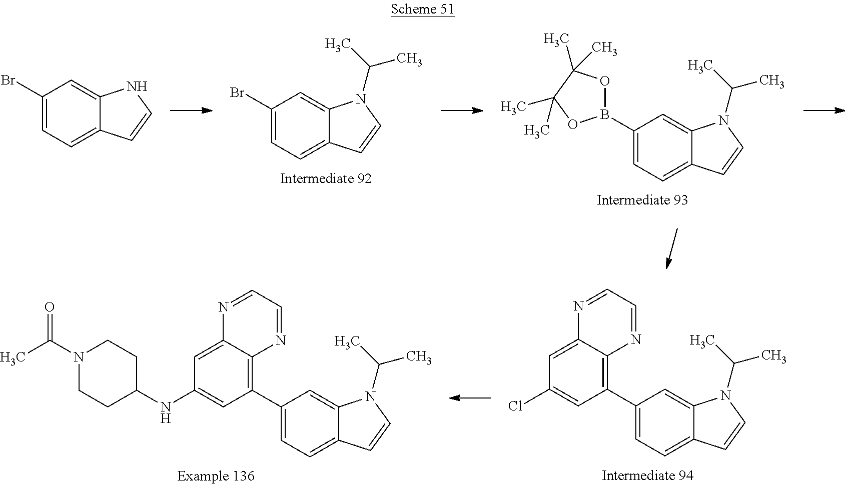 [SMILES: Brc1ccc2cc[nH]c2c1 ; CC(C)n1ccc2ccc(Br)cc21 ; CC(C)n1ccc2ccc(B3OC(C)(C)C(C)(C)O3)cc21 ; CC(=O)N1CCC(Nc2cc(-c3ccc4ccn(C(C)C)c4c3)c3nccnc3c2)CC1 ; CC(C)n1ccc2ccc(-c3cc(Cl)cc4nccnc34)cc21]

### Intermediate 92

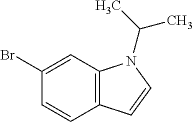

To an ice bath cooled solution of 6-Bromo-1H-indole (1.00 g; 5.10 mmol; 1.00 eq.) in anhydrous THF (10.00 ml) is added sodium hydride (60% in mineral oil) (0.24 g; 6.12 mmol; 1.20 eq.) under argon. The mixture is left with stirring for 30 minutes and 2-iodopropane (0.66 ml; 6.63 mmol; 1.30 eq.) was added dropwise at 0° C. The mixture is allowed to reach room temperature slowly, and then left with stirring at 60° C. under argon overnight. RM is poured onto ice and the mixture is extracted with Et₂O/Hexane 1/1 (3 times) The combined organic layers are washed with water, brine, dried over Na₂SO₄ and evaporated. The crude product is filtrated through a pad of silica gel, eluting with 4% AcOEt in hexanes to give 6-bromo-1-(propan-2-yl)-1H-indole (1.06 g; yield 83.8%; 96.00% by UPLC) as a light yellow oil.

### Intermediate 93

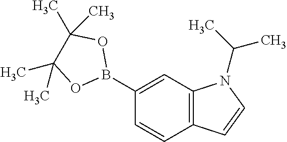 [SMILES: CC(C)n1ccc2ccc(B3OC(C)(C)C(C)(C)O3)cc21]

Product is prepared according to General Procedure 51 for Miyaura coupling borylation described for Intermediate 81 with 6-bromo-1-(propan-2-yl)-1H-indole (Intermediate 92) (1.00 g; 4.03 mmol; 1.00 eq.), bis(pinacolato)diboron (1.33 g; 5.24 mmol; 1.30 eq.), 1,4-dioxane (10.00 ml), Pd(dppf)Cl₂ (29.50 mg; 0.04 mmol; 0.01 eq.) and KOAc (0.79 g; 8.06 mmol; 2.00 eq.). Reaction is carried out overnight at 100° C. DCM is used to dilute RM. Purification by FCC (hexane/DCM; gradient) to give 1-(propan-2-yl)-6-(tetramethyl-1,3,2-dioxaborolan-2-yl)-1H-indole (586.00 mg; yield 46.9%; 92.00% by UPLC) as a colorless oil which crystallizes on standing.

### Intermediate 94

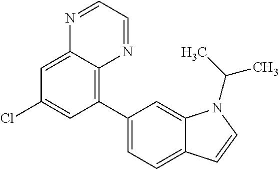 [SMILES: CC(C)n1ccc2ccc(-c3cc(Cl)cc4nccnc34)cc21]

The product is prepared according to General Procedure 1, described for Intermediate 4 with 1-(propan-2-yl)-6-(tetramethyl-1,3,2-dioxaborolan-2-yl)-1H-indole (Intermediate 93) (200 mg; 0.65 mmol; 1.00 eq.), 5-bromo-7-chloroquinoxaline (Intermediate 2) (157.88 mg; 0.65 mmol; 1.00 eq.), DIPEA (0.22 ml; 1.29 mmol; 2.00 eq.), Pd(dppf)Cl₂ (47.19 mg; 0.06 mmol; 0.10 eq.), 1,4-dioxane (2.50 ml) and water (2.50 ml). Reaction is carried out for 2.5 h 85° C. Then RM is diluted with EtOAc and filtered through Celite®. The filtrate is concentrated and the residue is purified by FCC (hexane/DCM; gradient then DCM/MeOH; gradient) to give 7-chloro-5-[1-(propan-2-yl)-1H-indol-6-yl]quinoxaline (141 mg; yield 67.2%; 98.9% by UPLC) as a yellow powder.

### Example 136

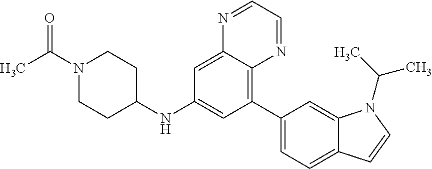 [SMILES: CC(=O)N1CCC(Nc2cc(-c3ccc4ccn(C(C)C)c4c3)c3nccnc3c2)CC1]

The product is prepared according to General Procedure 2, described in Example 1 with 1-(4-Aminopiperidin-1-yl)ethan-1-one hydrochloride (76.87 mg; 0.43 mmol; 2.00 eq.), NaOtBu (82.7 mg; 0.86 mmol; 4.00 eq.), Pd₂(dba)₃ (19.7 mg; 0.02 mmol; 0.10 eq.), BINAP (26.79 mg; 0.04 mmol; 0.20 eq.), 7-chloro-5-[1-(propan-2-yl)-1H-indol-6-yl]quinoxaline (Intermediate 94) (70 mg; 0.22 mmol; 1.00 eq.) and toluene (2.00 ml). Reaction is carried out overnight at 120° C. Then RM is diluted with AcOEt and filtered thorough Celite® and evaporated under reduced pressure. The residue is purified by FCC (DCM/MeOH; gradient) to give 1-[4-({8-[1-(propan-2-yl)-1H-indol-6-yl]quinoxalin-6-yl}amino)piperidin-1-yl]ethan-1-one (37.30 mg; yield 40.1%; 98.8% by HPLC) as a yellow green powder.

### Example 137

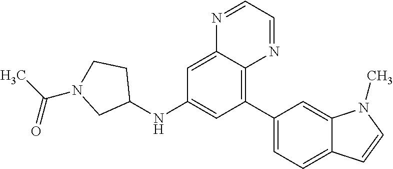 [SMILES: CC(=O)N1CCC(Nc2cc(-c3ccc4ccn(C)c4c3)c3nccnc3c2)C1]

The product is prepared according to General Procedure 2, described in Example 1 with 7-chloro-5-(1-methyl-1H-indol-6-yl)-quinoxaline (Intermediate 4) (50.00 mg; 0.17 mmol; 1.00 eq.), 1-[(3S)-3-aminopyrrolidin-1-yl]ethan-1-one (26.2 mg; 0.20 mmol; 1.2 eq.), NaOtBu (32.7 mg; 0.34 mmol; 2.00 eq.), BINAP (21.2 mg; 0.03 mmol; 0.20 eq.), Pd₂(dba)₃ (15.6 mg; 0.02 mmol; 0.10 eq.) and toluene (1.0 mL). Reaction is carried out in sealed tube at 120° C. for 18 h. Purification by FCC (MeOH/DCM; gradient). 1-[3-{[8-(1-methyl-1H-indol-6-yl)quinoxalin-6-yl]amino}pyrrolidin-1-yl]ethan-1-one (44.0 mg; 0.11 mmol; yield 65.7%; 97.9% by HPLC) is obtained as a yellow foam.

### Example 138

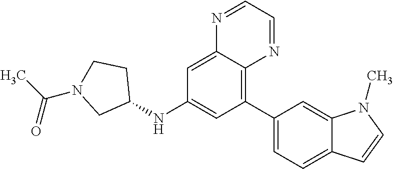 [SMILES: CC(=O)N1CC[C@H](Nc2cc(-c3ccc4ccn(C)c4c3)c3nccnc3c2)C1]

The product is prepared according to General Procedure 2, described in Example 1 with 7-chloro-5-(1-methyl-1H-indol-6-yl)-quinoxaline (Intermediate 4) (40.00 mg; 0.14 mmol; 1.00 eq.), 1-[(3S)-3-aminopyrrolidin-1-yl]ethan-1-one hydrochloride (56.49 mg; 0.34 mmol; 2.52 eq.), NaOtBu (52.35 mg; 0.54 mmol; 4.00 eq.), BINAP (16.96 mg; 0.03 mmol; 0.20 eq.), Pd₂(dba)₃ (12.47 mg; 0.01 mmol; 0.10 eq.) and toluene (1.20 mL). Reaction is carried out in sealed tube at 120° C. for 24 h. Purification by FCC (hexane/EtOAc; gradient). Repurification by preparative HPLC is done. 1-[(3S)-3-{[8-(1-methyl-1H-indol-6-yl)quinoxalin-6-yl]amino}pyrrolidin-1-yl]ethan-1-one (13.30 mg; yield 25.3%; 100% by HPLC) is obtained as a yellow powder.

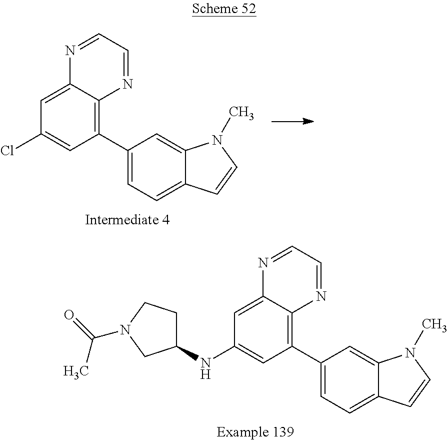 [SMILES: Cn1ccc2ccc(-c3cc(Cl)cc4nccnc34)cc21 ; CC(=O)N1CC[C@@H](Nc2cc(-c3ccc4ccn(C)c4c3)c3nccnc3c2)C1]

### Example 139

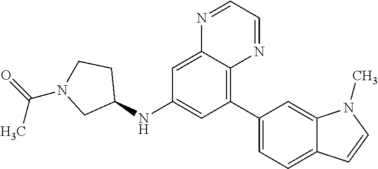 [SMILES: CC(=O)N1CC[C@@H](Nc2cc(-c3ccc4ccn(C)c4c3)c3nccnc3c2)C1]

The product is prepared according to General Procedure 2, described in Example 1 with 7-chloro-5-(1-methyl-1H-indol-6-yl)quinoxaline (Intermediate 4) (58.88 mg; 0.20 mmol; 0.50 eq.), Pd₂(dba)₃ (36.71 mg; 0.04 mmol; 0.10 eq.), NaOtBu (154.11 mg; 1.60 mmol; 4.00 eq.), BINAP (49.925 mg; 0.08 mmol; 0.20 eq.), 1-[(3R)-3-aminopyrrolidin-1-yl]ethan-1-one hydrochloride (66.00 mg; 0.40 mmol; 1.00 eq.) and toluene (1.50 ml). Reaction is carried out overnight at 120° C. Then it is diluted with EtOAc and DCM and filtered through Celite®. The filtrate is evaporated under reduced pressure and the residue is purified by FCC (DCM/MeOH; gradient) and preparative HPLC to give 1-[(3R)-3-{[8-(1-methyl-1H-indol-6-yl)quinoxalin-6-yl]amino}pyrrolidin-1-yl]ethan-1-one (16.00 mg; yield 10.3%; 99.0% by HPLC) as a yellow solid.

### Example 140

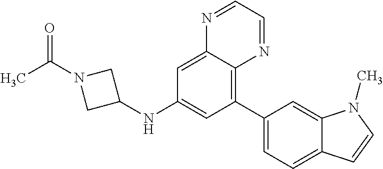 [SMILES: CC(=O)N1CC(Nc2cc(-c3ccc4ccn(C)c4c3)c3nccnc3c2)C1]

The product is prepared according to General Procedure 2, described in Example 1 with 7-chloro-5-(1-methyl-1H-indol-6-yl)-quinoxaline (Intermediate 4) (100.00 mg; 0.34 mmol; 1.00 eq.), 1-(3-aminoazetidin-1-yl)ethan-1-one (77.7 mg; 0.68 mmol; 2.0 eq.), NaOtBu (130.9 mg; 1.36 mmol; 4.00 eq.), BINAP (63.5 mg; 0.1 mmol; 0.30 eq.), Pd₂(dba)₃ (46.8 mg; 0.05 mmol; 0.15 eq.) and [1,4]-dioxane (1.20 mL). Reaction is carried out in sealed tube at 120° C. for 18 h. Purification by preparative HPLC (ACN/0.1% aquas ammonia, gradient). 1-(3-{[8-(1-Methyl-1H-indol-6-yl)quinoxalin-6-yl]amino}azetidin-1-yl)ethan-1-one (24.2 mg; 0.06 mmol; yield 19.1%; 99.7% by HPLC) is obtained as an orange-yellow glass.

### Example 141

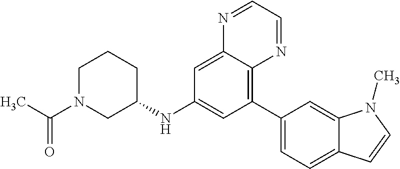 [SMILES: CC(=O)N1CCC[C@H](Nc2cc(-c3ccc4ccn(C)c4c3)c3nccnc3c2)C1]

The product is prepared according to General Procedure 2, with 7-chloro-5-(1-methyl-1H-indol-6-yl)-quinoxaline (Intermediate 4) (40.00 mg; 0.14 mmol; 1.00 eq.), (3S)-1-(3-aminopiperidin-1-yl)ethan-1-one hydrochloride (61.31 mg; 0.34 mmol; 2.52 eq.), NaOtBu (52.35 mg; 0.54 mmol; 4.00 eq.), BINAP (16.96 mg; 0.03 mmol; 0.20 eq.), Pd₂(dba)₃ (12.47 mg; 0.01 mmol; 0.10 eq.) and toluene (1.20 mL). Reaction is carried out in sealed tube at 120° C. for 24 h. Purification by FCC (hexane/EtOAc; gradient). Repurification by preparative HPLC is done. 1-[(3S)-3-{[8-(1-methyl-1H-indol-6-yl)quinoxalin-6-yl]amino}piperidin-1-yl]ethan-1-one (10.60 mg; yield 18.3%; 94.1% by HPLC) is obtained as a yellow powder.

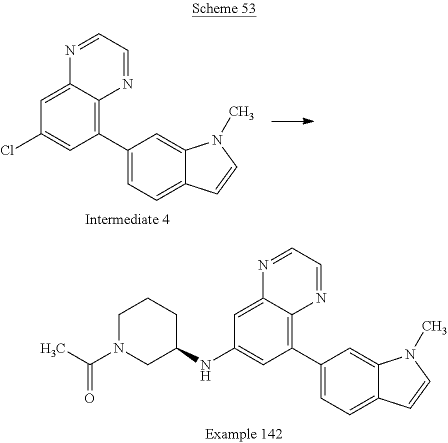 [SMILES: Cn1ccc2ccc(-c3cc(Cl)cc4nccnc34)cc21 ; CC(=O)N1CCC[C@@H](Nc2cc(-c3ccc4ccn(C)c4c3)c3nccnc3c2)C1]

### Example 142

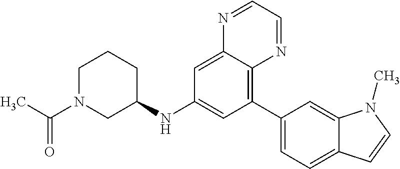 [SMILES: CC(=O)N1CCC[C@@H](Nc2cc(-c3ccc4ccn(C)c4c3)c3nccnc3c2)C1]

The product is prepared according to General Procedure 2, described in Example 1 with 7-chloro-5-(1-methyl-1H-indol-6-yl)quinoxaline (Intermediate 4) (40.00 mg; 0.14 mmol; 1.00 eq.), Pd₂(dba)₃ (12.47 mg; 0.01 mmol; 0.10 eq.), NaOtBu (52.35 mg; 0.54 mmol; 4.00 eq.), BINAP (16.96 mg; 0.03 mmol; 0.20 eq.), 1-[(3R)-3-aminopiperidin-1-yl]ethan-1-one hydrochloride (48.66 mg; 0.27 mmol; 2.00 eq.) and toluene (1.50 ml). Reaction is carried out for 3 h at 120° C. Then RM is diluted with AcOEt, filtered thorough Celite® and evaporated under reduced pressure. The residue is purified by FCC (DCM/MeOH; gradient) to give 1-[(3R)-3-{[8-(1-methyl-1H-indol-6-yl)quinoxalin-6-yl]amino}piperidin-1-yl]ethan-1-one (22.10 mg; yield 39.9%; 98.1% by HPLC) as a green yellow powder.

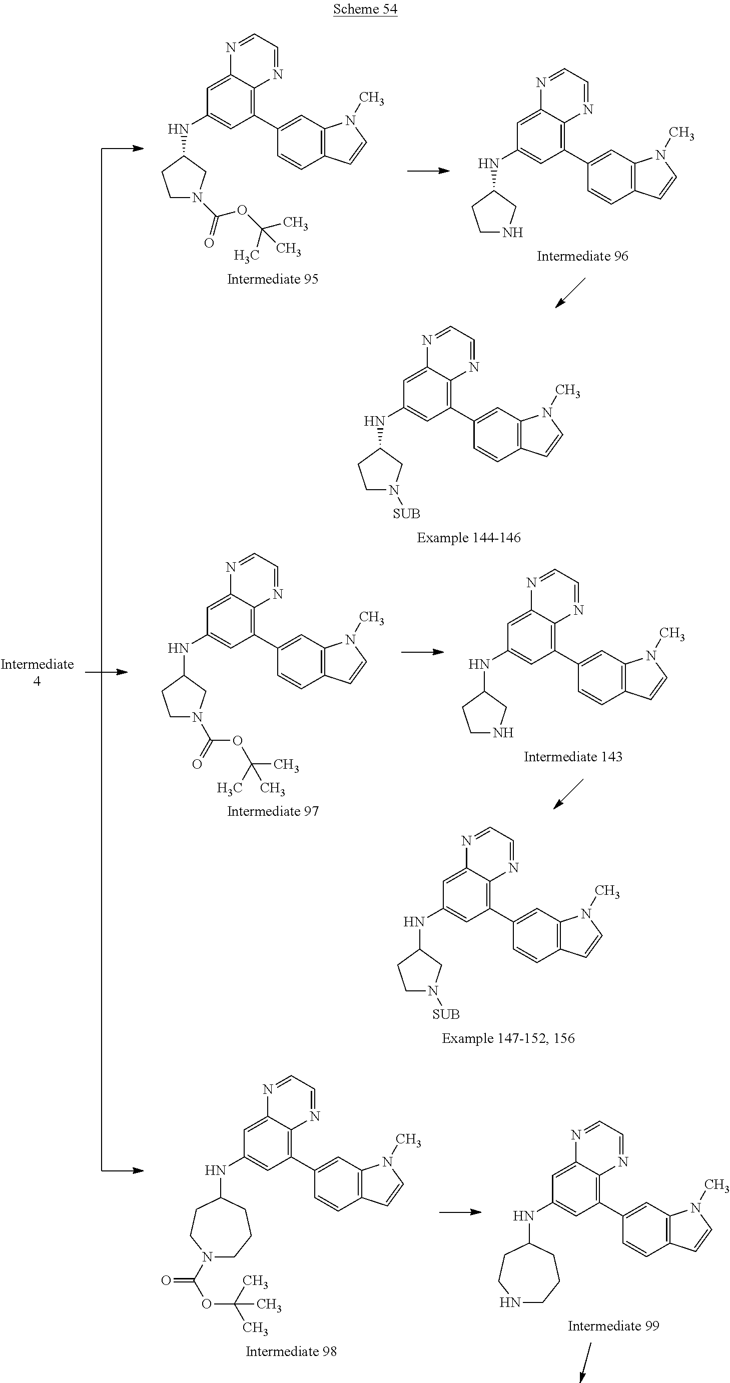

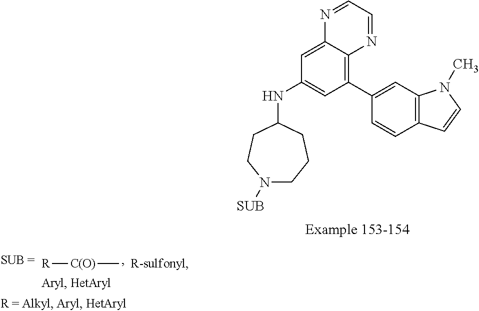

### Intermediate 95

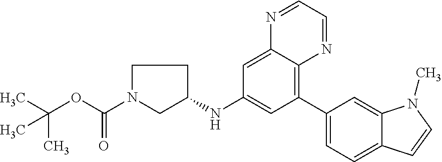 [SMILES: Cn1ccc2ccc(-c3cc(N[C@H]4CCN(C(=O)OC(C)(C)C)C4)cc4nccnc34)cc21]

The product is prepared according to modified General Procedure 2, described in Example 1 with 7-Chloro-5-(1-methyl-1H-indol-6-yl)quinoxaline (Intermediate 4) (0.400 g; 1.35 mmol; 1.00 eq.), tert-butyl (3S)-3-aminopyrrolidine-1-carboxylate (0.303 g; 1.63 mmol; 1.21 eq.), Pd₂(dba)₃ (0.123 g; 0.13 mmol; 0.10 eq.), NaOtBu (0.311 g; 3.24 mmol; 2.40 eq.), BINAP (0.168 g; 0.27 mmol; 0.20 eq.) and toluene (3.000 ml). Reaction is carried out overnight at 110° C. Then RM is diluted with EtOAc, DCM and filtered through Celite®. The filtrate is concentrated under reduced pressure and passed through a short pad of silica and the pad is washed with EtOAc. The solution is evaporated under reduced pressure and the residue is purified by FCC (DCM/MeOH; gradient) followed by FCC (hexane/EtOAc; gradient) to give tert-butyl (3S)-3-{[8-(1-methyl-1H-indol-6-yl)quinoxalin-6-yl]amino}pyrrolidine-1-carboxylate (586.30 mg; yield 97.2%; 99.2% by UPLC) as yellow amorphous solid.

### Intermediate 96—General Procedure 52

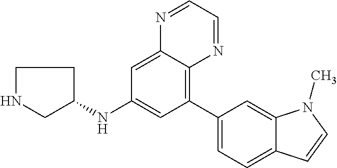 [SMILES: Cn1ccc2ccc(-c3cc(N[C@H]4CCNC4)cc4nccnc34)cc21]

Tert-butyl (3S)-3-{[8-(1-methyl-1H-indol-6-yl)quinoxalin-6-yl]amino}pyrrolidine-1-carboxylate (Intermediate 95) (582.60 mg; 1.30 mmol; 1.00 eq.), PTSA (495.71 mg; 2.61 mmol; 2.00 eq.), toluene (8.000 ml) and MeOH (2.000 ml) are placed in a MW reaction vessel. The vessel is capped and the air is evacuated and back filled with argon. RM is heated at MW 110° C. for 10 minutes. RM is diluted with toluene and a small volume of methanol then 2M NaOH is added. The resulting mixture is stirred vigorously and the stirring is continued after addition of EtOAc. The organic layer is washed with 2M NaOH, water, dried over anhydrous Na₂SO₄ and filtered. The filtrate is evaporated under reduced pressure to give 8-(1-methyl-1H-indol-6-yl)-N-[(3S)-pyrrolidin-3-yl]quinoxalin-6-amine (369.80 mg; yield 80.9%; 97.90% by UPLC) as a yellow foam.

### Intermediate 97

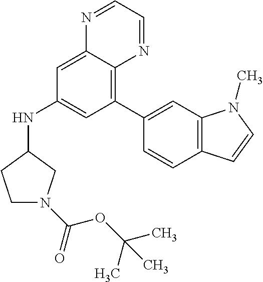 [SMILES: Cn1ccc2ccc(-c3cc(NC4CCN(C(=O)OC(C)(C)C)C4)cc4nccnc34)cc21]

The product is prepared according to General Procedure 2, described in Example 1 with 7-chloro-5-(1-methyl-1H-indol-6-yl)-quinoxaline (Intermediate 4) (200.0 mg; 0.68 mmol; 1.00 eq.), tert-butyl 3-aminopyrrolidine-1-carboxylate (151.8 mg; 0.28 mmol; 1.2 eq.), NaOtBu (156.7 mg; 1.63 mmol; 2.4 eq.), BINAP (84.6 mg; 0.14 mmol; 0.20 eq.), Pd₂(dba)₃ (62.8 mg; 0.07 mmol; 0.1 eq.) and [1,4]-dioxane (2.0 mL). Reaction is carried out in sealed tube at 120° C. for 18 h. Purification by FCC (MeOH/DCM, gradient). tert-Butyl 3-{[8-(1-methyl-1H-indol-6-yl)quinoxalin-6-yl]amino}pyrrolidine-1-carboxylate (Intermediate 95) (0.26 g; 0.56 mmol; yield 83.1%; 96.3% by UPLC) is obtained as yellow glass.

### Example 143

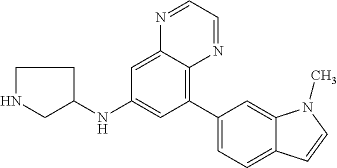 [SMILES: Cn1ccc2ccc(-c3cc(NC4CCNC4)cc4nccnc34)cc21]

The product is prepared according to General Procedure 52, described for Intermediate 96 with tert-butyl 3-{[8-(1-methyl-1H-indol-6-yl)quinoxalin-6-yl]amino}pyrrolidine-1-carboxylate (Intermediate 97) (40.00 mg; 0.08 mmol; 1.00 eq.), PTSA monohydrate (31.77 mg; 0.17 mmol; 2.00 eq.) and toluene (2.00 ml). Reaction is carried out at MW 100-110° C. for 5 min. Then 2M NaOH is added and the resulting mixture is extracted with EtOAc. The aqueous layer is extracted with DCM. The combined organic layers are dried over anhydrous Na₂SO₄ and filtered. The filtrate is evaporated under reduced pressure and the residue is purified by FCC (hexane/DCM; gradient then DCM/MeOH; gradient, NH₂-silica) to give 8-(1-methyl-1H-indol-6-yl)-N-(pyrrolidin-3-yl)quinoxalin-6-amine (12.90 mg; yield 42.4%; 94.20% by HPLC) as a yellow powder.

### Intermediate 98

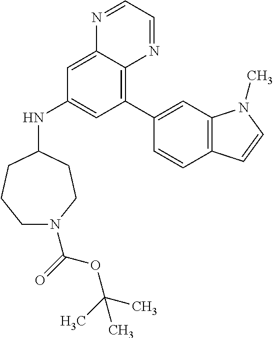 [SMILES: Cn1ccc2ccc(-c3cc(NC4CCCN(C(=O)OC(C)(C)C)CC4)cc4nccnc34)cc21]

The product is prepared according to General Procedure 2, described in Example 1 with 7-chloro-5-(1-methyl-1H-indol-6-yl)-quinoxaline (Intermediate 4) (89 mg; 0.30 mmol; 1.00 eq.), tert-butyl 4-aminoazepane-1-carboxylate (85.0 mg; 0.40 mmol; 1.3 eq.), NaOtBu (60.0 mg; 0.62 mmol; 2.0 eq.), BINAP (37.0 mg; 0.06 mmol; 0.20 eq.), Pd₂(dba)₃ (27.0 mg; 0.03 mmol; 0.1 eq.) and [1,4]-dioxane (2.0 mL). Reaction is carried out in sealed tube at 120° C. for 18 h. Purification by FCC (MeOH/DCM, gradient). tert-Butyl 4-{[8-(1-methyl-1H-indol-6-yl)quinoxalin-6-yl]amino}azepane-1-carboxylate (Intermediate 96) (0.111 g; 0.23 mmol; yield 76.3%; 98% by UPLC) is obtained as yellow-brownish solid.

### Intermediate 99

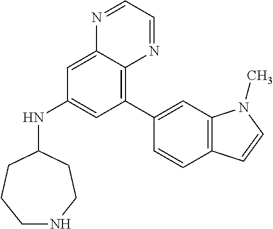 [SMILES: Cn1ccc2ccc(-c3cc(NC4CCCNCC4)cc4nccnc34)cc21]

The product is prepared according to General Procedure 52 for MW BOC-deprotection, described for Intermediate 96 with with tert-Butyl 4-{[8-(1-methyl-1H-indol-6-yl)quinoxalin-6-yl]amino}azepane-1-carboxylate (Intermediate 96) (0.111 g; 0.23 mmol; 1.0 eq), PTSA (89.5 mg; 0.47 mmol; 2.0 eq.), anhydrous toluene (4.00 ml) and anhydrous methanol (1.00 ml). The vessel is sealed and RM is heated to 100° C. and irradiated with MW in the Biotage Initiator unit for 10 min. Purification by pH dependent extraction. Crude N-(azepan-4-yl)-8-(1-methyl-1H-indol-6-yl)quinoxalin-6-amine (Intermediate 97) (78.40 mg; 0.20 mmol; yield 83.2%; 92.8% by UPLC) is obtained as yellow-greenish foam.

### Example 144

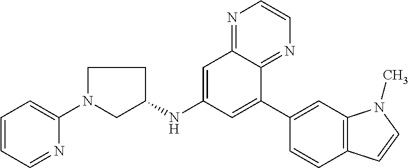 [SMILES: Cn1ccc2ccc(-c3cc(N[C@H]4CCN(c5ccccn5)C4)cc4nccnc34)cc21]

8-(1-methyl-1H-indol-6-yl)-N-[(3S)-pyrrolidin-3-yl]quinoxalin-6-amine (Intermediate 96) (40.00 mg; 0.11 mmol; 1.00 eq.), 2-fluoropyridine (0.010 ml; 0.11 mmol; 1.00 eq.), potassium carbonate (17.34 mg; 0.13 mmol; 1.10 eq.) and ACN (1.000 ml) are placed in a MW reaction vessel and the resulting mixture is purged with argon. Then the vessel is capped and RM is heated at MW 150° C. for 3.5 h. Then 8-(1-methyl-1H-indol-6-yl)-N-[(3S)-pyrrolidin-3-yl]quinoxalin-6-amine (Intermediate 96) (20.00 mg; 0.06 mmol; 0.50 eq.) and ACN (0.500 ml) is added and RM is purged again with argon and heated at MW 150° C. for additional 3 h. RM is evaporated under reduced pressure and the residue is purified by FCC (DCM/MeOH; gradient) to give 8-(1-methyl-1H-indol-6-yl)-N-[(3S)-1-(pyridin-2-yl)pyrrolidin-3-yl]quinoxalin-6-amine (15.90 mg; yield 32.9%; 99.30% by HPLC) as a yellow powder.

### Example 145

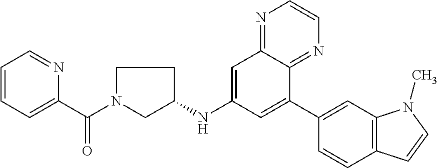 [SMILES: Cn1ccc2ccc(-c3cc(N[C@H]4CCN(C(=O)c5ccccn5)C4)cc4nccnc34)cc21]

To a mixture of 8-(1-methyl-1H-indol-6-yl)-N-[(3S)-pyrrolidin-3-yl]quinoxalin-6-amine (Intermediate 96) (32.00 mg; 0.09 mmol; 1.00 eq.), DIPEA (0.048 ml; 0.28 mmol; 3.02 eq.) and DCM (2.000 ml) placed in an ice bath pyridine-2-carbonyl chloride hydrochloride (17.05 mg; 0.10 mmol; 1.05 eq.) is added. RM is stirred for 2 h at rt. Then RM is placed again in an ice bath and DCM (0.500 ml), DIPEA (0.500 ml; 2.87 mmol; 31.47 eq.) and pyridine-2-carbonyl chloride hydrochloride (19 mg; 0.11 mmol; 1.17 eq.) are added. The resulting mixture is stirred overnight at rt. Then water is added followed by DCM. The organic layer is washed with water, brine, dried over anhydrous Na₂SO₄ and filtered. The filtrate is evaporated under reduced pressure and the residue is purified by FCC (DCM/MeOH; gradient) to give 8-(1-methyl-1H-indol-6-yl)-N-[(3S)-1-(pyridine-2-carbonyl)pyrrolidin-3-yl]quinoxalin-6-amine (2.50 mg; yield 6.0%; 98.70% by HPLC) as a yellow film.

### Example 146

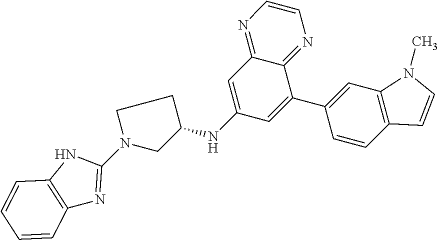 [SMILES: Cn1ccc2ccc(-c3cc(N[C@H]4CCN(c5nc6ccccc6[nH]5)C4)cc4nccnc34)cc21]

2-Bromo-1H-benzoimidazole (17.00 mg; 0.09 mmol; 1.00 eq.), 8-(1-methyl-1H-indol-6-yl)-N-[(3S)-pyrrolidin-3-yl]quinoxalin-6-amine (Intermediate 96) (38.95 mg; 0.11 mmol; 1.30 eq.), TEA (0.032 ml; 0.23 mmol; 2.70 eq.) and DMF (1.000 ml) are placed in a reaction vessel and the resulting mixture is purged with argon. Then the vessel is closed and RM is heated overnight at 100° C. with stirring and then again overnight at 110° C. RM is evaporated under reduced pressure and the residue is purified by FCC (DCM/MeOH; gradient) and preparative HPLC. The fraction is concentrated under reduced pressure and DCM is added followed by 2M NaOH with stirring. The organic layer is washed with water and evaporated under reduced pressure to give N-[(3S)-1-(1H-1,3-benzodiazol-2-yl)pyrrolidin-3-yl]-8-(1-methyl-1H-indol-6-yl)-quinoxalin-6-amine (8.50 mg; yield 21.6%; 99.80% by HPLC) as a yellow powder.

### Example 147

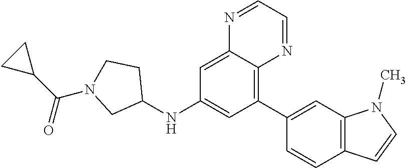 [SMILES: Cn1ccc2ccc(-c3cc(NC4CCN(C(=O)C5CC5)C4)cc4nccnc34)cc21]

The roundbottom flask is charged with 8-(1-methyl-1H-indol-6-yl)-N-(pyrrolidin-3-yl)quinoxalin-6-amine (example 143) (25 mg; 0.07 mmol; 1.0 eq.), DIPEA (25 μl; 0.14 mmol; 2.00 eq.) and anhydrous DCM (2.0 ml). Resulted mixture is cooled to 0° C. in an ice bath. The cyclopropanecarbonyl chloride (7.0 μl; 0.07 mmol; 1.00 eq.) is added through syringe under inert atmosphere. RM is stirred overnight, then solvent is evaporated in vacuo. Purification by FCC (MeOH/DCM, gradient). N-(1-cyclopropanecarbonylpyrrolidin-3-yl)-8-(1-methyl-1H-indol-6-yl)quinoxalin-6-amine (23.5 mg; 0.06 mmol; yield 77.3%; 97.6% by HPLC) is obtained as green-yellow foam.

### Example 148

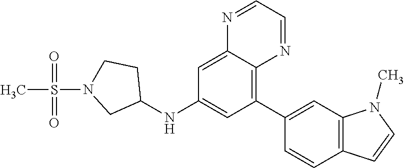 [SMILES: Cn1ccc2ccc(-c3cc(NC4CCN(S(C)(=O)=O)C4)cc4nccnc34)cc21]

The roundbottom flask is charged with 8-(1-methyl-1H-indol-6-yl)-N-(pyrrolidin-3-yl)quinoxalin-6-amine (Example 134) (40 mg; 0.12 mmol; 1.0 eq.), DIPEA (40 μl; 0.23 mmol; 2.00 eq.) and anhydrous DCM (2.0 ml). Resulted mixture is cooled to −10° C. in an salt-ice bath. The methanesulfonyl chloride (9.0 μl; 0.12 mmol; 1.00 eq.) is added through syringe under inert atmosphere. RM is stirred overnight, then solvent is evaporated in vacuo. Purification by FCC (MeOH/DCM, gradient). N-(1-Methanesulfonylpyrrolidin-3-yl)-8-(1-methyl-1H-indol-6-yl)quinoxalin-6-amine (43.2 mg; 0.10 mmol; yield 87.5%; 98.6% by HPLC) is obtained as green-yellow foam.

### Example 149

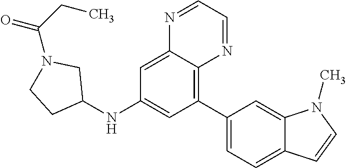 [SMILES: CCC(=O)N1CCC(Nc2cc(-c3ccc4ccn(C)c4c3)c3nccnc3c2)C1]

The product is prepared according to General Procedure 35, described in Example 82 with 8-(1-methyl-1H-indol-6-yl)-N-(pyrrolidin-3-yl)quinoxalin-6-amine (Example 143) (58.00 mg; 0.15 mmol; 1.00 eq.), TEA (0.100 ml; 0.72 mmol; 4.84 eq.), propanoyl chloride (0.013 ml; 0.15 mmol; 1.00 eq.) and DCM (2.000 ml). Reaction is carried out for 2 h at 0° C. Purification by FCC (DCM/MeOH; gradient) (twice) to give 1-(3-{[8-(1-methyl-1H-indol-6-yl)-quinoxalin-6-yl]amino}pyrrolidin-1-yl)propan-1-one (25.40 mg; yield 39.9%; 93.00% by HPLC) as a yellow powder.

### Example 150

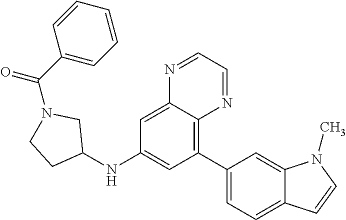 [SMILES: Cn1ccc2ccc(-c3cc(NC4CCN(C(=O)c5ccccc5)C4)cc4nccnc34)cc21]

To a mixture of 8-(1-methyl-1H-indol-6-yl)-N-(pyrrolidin-3-yl)quinoxalin-6-amine (Example 143) (31.00 mg; 0.09 mmol; 1.00 eq.), DIPEA (0.200 ml; 1.14 mmol; 13.12 eq.) and DCM (2.000 ml) benzoyl chloride (0.011 ml; 0.09 mmol; 1.05 eq.) is added and RM is stirred for 2 h at rt. Then solution of NaHCO₃ is added followed by DCM. The organic phase is washed with water, brine, dried over anhydrous MgSO₄ and filtered. The filtrate is evaporated under reduced pressure and the residue is purified by FCC (DCM/MeOH; gradient) and preparative HPLC. Fractions are concentrated under reduced pressure then 2M NaOH is added followed by DCM with mixing. The organic phase is washed with water, dried over anhydrous Na₂SO₄ and filtered. The filtrate is evaporated under reduced pressure to give N-(1-benzoylpyrrolidin-3-yl)-8-(1-methyl-1H-indol-6-yl)quinoxalin-6-amine (19.00 mg; yield 48.9%; 99.80% by HPLC) as a yellow powder.

### Example 151

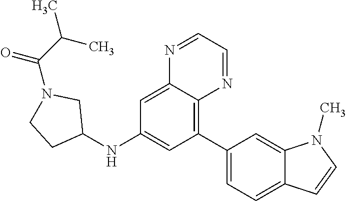 [SMILES: CC(C)C(=O)N1CCC(Nc2cc(-c3ccc4ccn(C)c4c3)c3nccnc3c2)C1]

To a mixture of 8-(1-methyl-1H-indol-6-yl)-N-(pyrrolidin-3-yl)quinoxalin-6-amine (Example 143) (33.00 mg; 0.09 mmol; 1.00 eq.), DIPEA (0.100 ml; 0.57 mmol; 6.01 eq.) and DCM (2.000 ml) placed in an ice bath 2-methylpropanoyl chloride (0.010 ml; 0.09 mmol; 1.00 eq.) in DCM (1.000 ml) is added and RM is stirred overnight at rt. Then solution of NaHCO₃ is added followed by DCM. The organic layer is washed with water, brine, dried over anhydrous MgSO4 and filtered. The filtrate is evaporated under reduced pressure and the residue is purified by FCC (DCM/MeOH; gradient) to give 2-methyl-1-(3-{[8-(1-methyl-1H-indol-6-yl)quinoxalin-6-yl]amino}pyrrolidin-1-yl)propan-1-one (17.60 mg; yield 43.7%; 97.20% by HPLC) as a yellow powder.

### Example 152

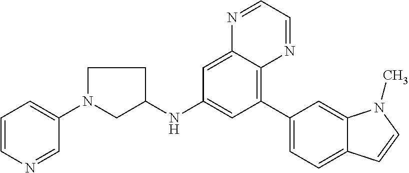 [SMILES: Cn1ccc2ccc(-c3cc(NC4CCN(c5cccnc5)C4)cc4nccnc34)cc21]

The product is prepared according to General Procedure 5, described in Example 30 with 3-bromopyridine (0.008 ml; 0.08 mmol; 1.00 eq.), 8-(1-methyl-1H-indol-6-yl)-N-(pyrrolidin-3-yl)quinoxalin-6-amine (Example 143) (26.48 mg; 0.08 mmol; 1.00 eq.), BippyPhos (3.08 mg; 0.01 mmol; 0.08 eq.), t-BuONa (16.79 mg; 0.17 mmol; 2.30 eq.), [(Cinnamyl)PdCl]₂ (1.97 mg; 0.008 mmol; 0.05 eq.) and toluene (1.500 ml). Reaction is carried out for 3 h at 110° C. Then RM is diluted with EtOAc, DCM and filtered through Celite®. The filtrate is evaporated under reduced pressure and the residue is purified by FCC (DCM/MeOH; gradient) to give 8-(1-methyl-1H-indol-6-yl)-N-[1-(pyridine-3-yl)pyrrolidin-3-yl]quinoxalin-6-amine (3.00 mg; yield 8.8%; 93.90% by HPLC) as a yellow solid.

### Example 153

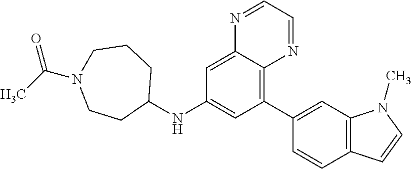 [SMILES: CC(=O)N1CCCC(Nc2cc(-c3ccc4ccn(C)c4c3)c3nccnc3c2)CC1]

The roundbottom flask is charged with N-(azepan-4-yl)-8-(1-methyl-1H-indol-6-yl)quinoxalin-6-amine (Intermediate 99) (35 mg; 0.09 mmol; 1.0 eq.), DIPEA (30 μl; 0.17 mmol; 2.00 eq.) and anhydrous DCM (2.0 ml). Resulted mixture is cooled to 0° C. in an ice bath. The acetyl chloride (7.0 μl; 0.10 mmol; 1.00 eq.) is added through syringe under inert atmosphere. RM is stirred 1 h at rt, then solvent is evaporated in vacuo. Purification by FCC (MeOH/DCM, gradient). 1-(4-{[8-(1-Methyl-1H-indol-6-yl)quinoxalin-6-yl]amino}azepan-1-yl)ethan-1-one (29.0 mg; 0.07 mmol; yield 78.0%; 97.2% by HPLC) is obtained as yellow glass.

### Example 154

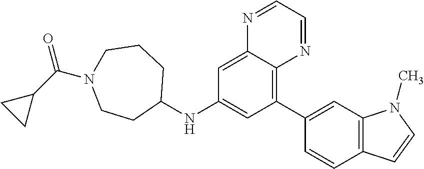 [SMILES: Cn1ccc2ccc(-c3cc(NC4CCCN(C(=O)C5CC5)CC4)cc4nccnc34)cc21]

The roundbottom flask is charged with N-(azepan-4-yl)-8-(1-methyl-1H-indol-6-yl)quinoxalin-6-amine (Intermediate 99) (35 mg; 0.09 mmol; 1.0 eq.), DIPEA (30 μl; 0.17 mmol; 2.00 eq.) and anhydrous DCM (2.0 ml). Resulted mixture is cooled to 0° C. in an ice bath. The cyclopropanecarbonyl chloride (9.0 μl; 0.01 mmol; 1.1 eq.) is added through syringe under inert atmosphere. RM is stirred overnight, then solvent is evaporated in vacuo. Purification by FCC (MeOH/DCM, gradient). N-(1-cyclopropanecarbonylazepan-4-yl)-8-(1-methyl-1H-indol-6-yl)quinoxalin-6-amine (34.0 mg; 0.08 mmol; yield 88.5%; 96.0% by HPLC) is obtained as yellow glass.

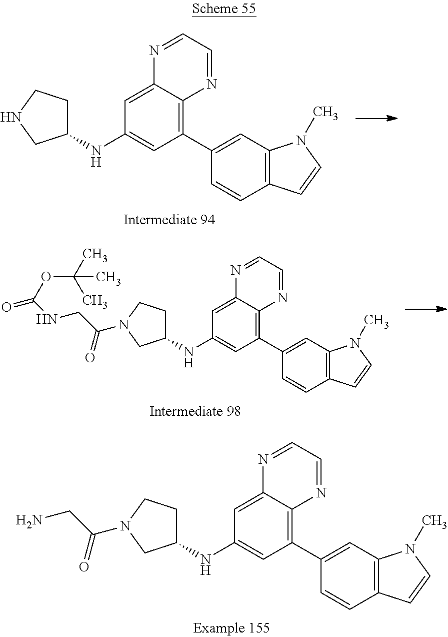 [SMILES: Cn1ccc2ccc(-c3cc(N[C@H]4CCNC4)cc4nccnc34)cc21 ; Cn1ccc2ccc(-c3cc(N[C@H]4CCN(C(=O)CNC(=O)OC(C)(C)C)C4)cc4nccnc34)cc21 ; Cn1ccc2ccc(-c3cc(N[C@H]4CCN(C(=O)CN)C4)cc4nccnc34)cc21]

### Intermediate 100

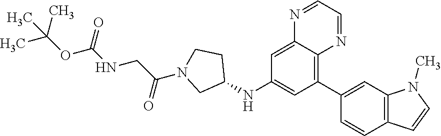 [SMILES: Cn1ccc2ccc(-c3cc(N[C@H]4CCN(C(=O)CNC(=O)OC(C)(C)C)C4)cc4nccnc34)cc21]

A sealed tube is charged with 8-(1-methyl-1H-indol-6-yl)-N-(pyrrolidin-3-yl)quinoxalin-6-amine (35.00 mg; 0.10 mmol; 1.0 eq.) (Intermediate 96), HBTU (46.38 mg; 0.12 mmol; 1.2 eq.) and 2-{[(tert-butoxy)carbonyl]amino}-acetic acid (17.85 mg; 0.10 mmol; 1.0 eq.). Tube is closed and air is evacuated to the vacuum and content is backfilled with argon. The cycle is repeated three times. Anhydrous dimethylformamide (2.0 mL) is added under argon atmosphere along with DIPEA (26.34 mg; 0.20 mmol; 2.0 eq.) and content is stirred for 24h. After this time reaction is diluted with DCM (10 mL) and washed with 30% aqueous citric acid (10 mL), brine (10 mL) and saturated aqueous solution of NaHCO₃ (10 mL). The DCM phase is dried over Na₂SO₄ for 24h. Organic solvent is then evaporated to afford tert-butyl N-[2-(3-{[8-(1-methyl-1H-indol-6-yl)quinoxalin-6-yl]amino}pyrrolidin-1-yl)-2-oxoethyl]carbamate (Intermediate 98) (54.00 mg; yield 83.7%; 79.1% by UPLC) as green powder.

### Example 155

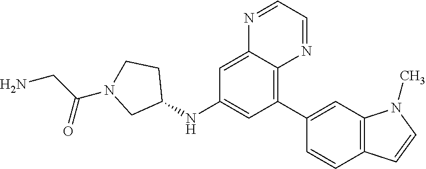 [SMILES: Cn1ccc2ccc(-c3cc(N[C@H]4CCN(C(=O)CN)C4)cc4nccnc34)cc21]

Microwave reactor vessel is charged with tert-butyl N-[2-(3-{[8-(1-methyl-1H-indol-6-yl)quinoxalin-6-yl]amino}pyrrolidin-1-yl)-2-oxoethyl] carbamate (intermediate 100) (25.00 mg; 0.05 mmol; 1.0 eq.), PTSA (17.82 mg; 0.09 mmol; 2.0 eq.), anhydrous toluene (1.00 mL) and anhydrous methanol (0.50 mL). Vessel is capped and air is evacuated to the vacuum and content is backfilled with argon. Vessel is heated at 110° C. under microwave irradiation for 10 minutes. After this time RM is diluted with a toluene (5 mL) and a small amount of methanol. 2M aq. NaOH is added and phases are stirred vigorously. After 5 minutes ethyl acetate (10 mL) is added. The organic layer is separated, washed with aq. 2 M NaOH (10 mL), water (10 mL) and dried over anhydrous Na₂SO₄ for 24h. Organic solvent is then evaporated to afford 2-Amino-1-[(3S)-3-{[8-(1-methyl-1H-indol-6-yl)quinoxalin-6-yl]amino}pyrrolidin-1-yl]ethan-1-one (19.30 mg; yield 97.0%; 94.3% by HPLC) as yellow solid.

### Example 156

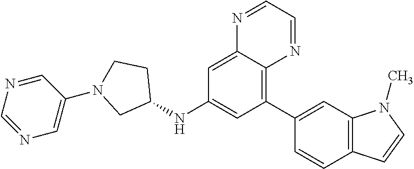 [SMILES: Cn1ccc2ccc(-c3cc(N[C@H]4CCN(c5cncnc5)C4)cc4nccnc34)cc21]

Sealed tube is charged with 8-(1-methyl-1H-indol-6-yl)-N-(pyrrolidin-3-yl)quinoxalin-6-amine (43 mg; 0.12 mmol; 1.0 eq.), NaOtBu (74 mg; 0.77 mmol; 6.2 eq.), 5-Bromopyrimidine (50 mg; 0.31 mmol; 2.5 eq.) and AdBrettPhos Pd G3 (3.00 mg; 0.00 mmol; 0.02 eq.). The tube is sealed with PTFE-coated silicone cap. Air from tube is evacuated invacuo and backfilled with argon (cycle is repeated three times) and anhydrous toluene is injected through syringe. Resulted mixture is stirred and heated for 18 h at 120° C. RM is diluted with EtOAc and filtered through Celite® pad. Filtrate is evaporated to dark oil which is purified by FCC (MeOH/EtOAc, gradient). 8-(1-Methyl-1H-indol-6-yl)-N-[(3S)-1-(pyrimidin-5-yl)pyrrolidin-3-yl]quinoxalin-6-amine (12.7 mg; 0.03 mmol; yield 23.0%; 93.5% by HPLC) is obtained as orange-yellow solid.

### Example 157

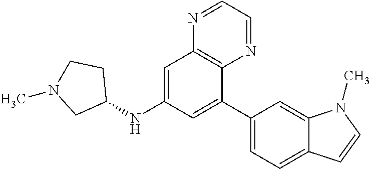 [SMILES: CN1CC[C@H](Nc2cc(-c3ccc4ccn(C)c4c3)c3nccnc3c2)C1]

The product is prepared according to General Procedure 2, described in Example 1 with 7-chloro-5-(1-methyl-1H-indol-6-yl)-quinoxaline (Intermediate 4) (50.00 mg; 0.17 mmol; 1.00 eq.), (3S)-1-methylpyrrolidin-3-amine hydrochloride (69.76 mg; 0.51 mmol; 3.00 eq.), NaOtBu (81.79 mg; 0.85 mmol; 5.00 eq.), BINAP (21.20 mg; 0.03 mmol; 0.20 eq.), Pd₂(dba)₃ (15.59 mg; 0.02 mmol; 0.10 eq.) and toluene (1.25 mL). Reaction is carried out in sealed tube at 120° C. for 24 h. Purification by FCC (DCM/MeOH; gradient). Repurification by preparative HPLC is done. 8-(1-methyl-1H-indol-6-yl)-N-[(3S)-1-methylpyrrolidin-3-yl]quinoxalin-6-amine (22.80 mg; yield 35.7%; 95.2% by HPLC) is obtained as a brown green solid.

### Example 158

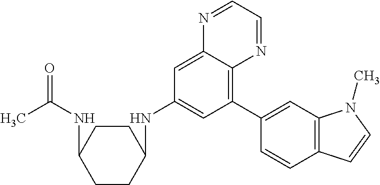 [SMILES: CC(=O)NC1CCC(Nc2cc(-c3ccc4ccn(C)c4c3)c3nccnc3c2)CC1]

The product is prepared according to modified General Procedure 2, described in Example 1 with 7-chloro-5-(1-methyl-1H-indol-6-yl)-quinoxaline (Intermediate 4) (50.00 mg; 0.17 mmol; 1.00 eq.), tert-butyl N-({1,4-cis}-4-aminocyclohexyl)carbamate 36.40 mg; 0.17 mmol; 1.00 eq.), NaOtBu (36.6 mg; 0.34 mmol; 2.00 eq.), BINAP (10.6 mg; 0.02 mmol; 0.10 eq.), Pd₂(dba)₃ (7.8 mg; 0.01 mmol; 0.05 eq.) and [1,4]-dioxane (1.0 mL). Reaction is carried out in sealed tube at 110° C. for 18 h. RM is cooled to rt and acetyl chloride (31 μl; 0.42 mmol; 2.50 eq.) is added through syringe. Resulted slurry is stirred for additional 2 h at rt. Then RM is diluted with EtOAC and filtered by Celite® pad. Filtrate is evaporated to give green foam. Purification by FCC (DCM/MeOH; gradient). N-({1,4-cis}-4-{[8-(1-Methyl-1H-indol-6-yl)quinoxalin-6-yl]amino}cyclohexyl)acetamide (10.0 mg; 0.02 mmol; yield 13.9%; 97.9% by HPLC) is obtained as a yellow-rgreen glass.

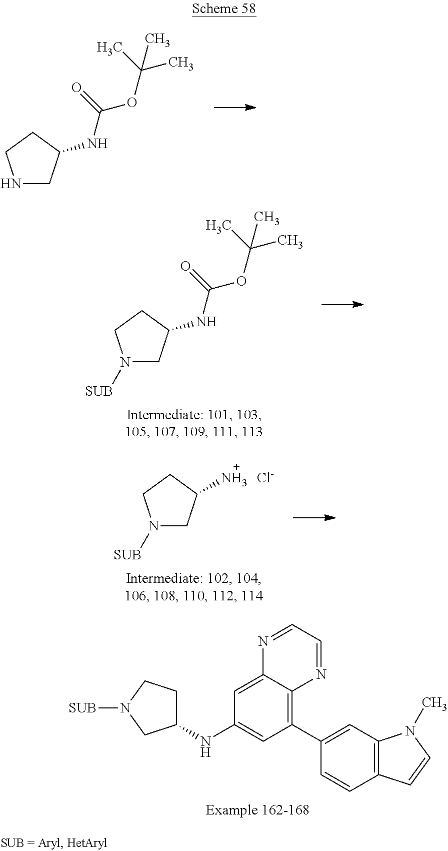

### Intermediate 101

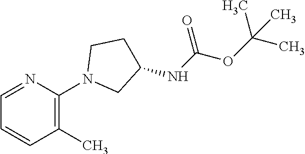 [SMILES: Cc1cccnc1N1CC[C@H](NC(=O)OC(C)(C)C)C1]

A sealed tube is charged with tert-butyl N-[(3S)-pyrrolidin-3-yl]carbamate (729.99 mg; 3.92 mmol; 2.5 eq.), 2-chloro-3-methylpyridine (200 mg; 1.57 mmol; 1.0 eq.) and triethylamine (0.66 mL; 4.70 mmol; 3.0 eq.). RM is sealed and heated at 130° C. for 24h. After this time, the mixture is diluted with DCM and is purified by FCC (DCM/MeOH; gradient) to afford tert-butyl N-[(3S)-1-(3-methylpyridin-2-yl)pyrrolidin-3-yl]carbamate (Intermediate 101) (143.10 mg; yield 32.5%; 98.8% by UPLC) as a brown solid.

### Intermediate 102—General Procedure 53

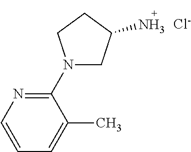

Roundbottom flask is charged with afford tert-butyl N-[(3S)-1-(3-methylpyridin-2-yl)pyrrolidin-3-yl]carbamate (Intermediate 101) (143.10 mg; 0.51 mmol; 1.0 eq.) and anhydrous Et₂O (7.16 mL). RM is cooled down to 0° C. and 2M HCl in Et₂O (0.76 mL; 1.53 mmol; 3.0 eq.) is added dropwise in this temperature. RM is stirred at room temperature for 24 h. After this time solvent is evaporated to afford (3S)-1-(3-methylpyridin-2-yl)pyrrolidin-3-amine hydrochloride (Intermediate 102) (143.30 mg; yield 97.0%; 86.3% by UPLC) as a beige solid.

### Example 162

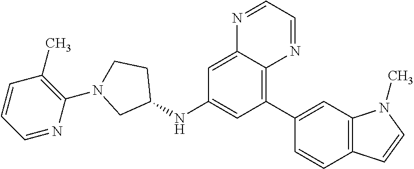 [SMILES: Cc1cccnc1N1CC[C@H](Nc2cc(-c3ccc4ccn(C)c4c3)c3nccnc3c2)C1]

The product is prepared according to General Procedure 2, described in Example 1 with 7-chloro-5-(1-methyl-1H-indol-6-yl)-quinoxaline (Intermediate 4) (50.00 mg; 0.17 mmol; 1.00 eq.), (3S)-1-(3-methylpyridin-2-yl)pyrrolidin-3-amine hydrochloride (Intermediate 102) (109.13 mg; 0.34 mmol; 3.00 eq.), NaOtBu (81.79 mg; 0.85 mmol; 5.00 eq.), BINAP (21.20 mg; 0.03 mmol; 0.20 eq.), Pd₂(dba)₃ (15.59 mg; 0.02 mmol; 0.10 eq.) and toluene (1.20 mL). Reaction is carried out in sealed tube at 120° C. for 24 h. Purification by FCC (DCM/MeOH; gradient). Repurification by preparative HPLC is done. 8-(1-methyl-1H-indol-6-yl)-N-[(3S)-1-(3-methylpyridin-2-yl)pyrrolidin-3-yl]quinoxalin-6-amine (30.80 mg; yield 40.7%; 97.8% by HPLC) is obtained as a green powder.

### Intermediate 103

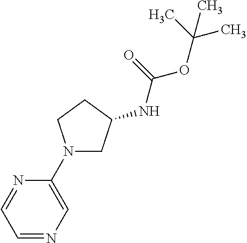 [SMILES: CC(C)(C)OC(=O)N[C@H]1CCN(c2cnccn2)C1]

A sealed tube is charged with tert-butyl N-[(3S)-pyrrolidin-3-yl]carbamate (813.09 mg; 4.37 mmol; 5.0 eq.), 2-chloropyrazine (100 mg; 0.87 mmol; 1.0 eq.) and triethylamine (0.37 mL; 2.62 mmol; 3.0 eq.). RM is sealed and heated at 130° C. for 24h. After this time, the mixture is diluted with DCM and is purified by FCC (DCM/MeOH; gradient) to afford tert-butyl N-[(3S)-1-(pyrazin-2-yl)pyrrolidin-3-yl]carbamate (Intermediate 101) (178.50 mg; yield 77.3%; 100% by UPLC) as a brown solid.

### Intermediate 104

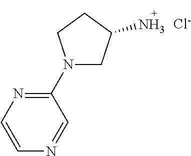 [SMILES: N[C@H]1CCN(c2cnccn2)C1 ; [Cl-]]

The product is prepared according to General Procedure 53, described for Intermediate 102 with tert-butyl N-[1-(pyrazin-2-yl)pyrrolidin-3-yl]carbamate (Intermediate 103) (178.50 mg; 0.68 mmol; 1.0 eq.), anhydrous Et₂O (8.93 mL) and 2M HCl in Et₂O (1.01 mL; 2.03 mmol; 3.0 eq.). Solvent is evaporated to afford (3S)-1-(pyrazin-2-yl)pyrrolidin-3-amine hydrochloride (Intermediate 104) (158.00 mg; yield 98.7%; 100% by UPLC) as a light brown solid.

### Example 163

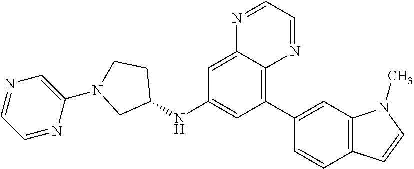 [SMILES: Cn1ccc2ccc(-c3cc(N[C@H]4CCN(c5cnccn5)C4)cc4nccnc34)cc21]

The product is prepared according to General Procedure 2, described in Example 1 with 7-chloro-5-(1-methyl-1H-indol-6-yl)-quinoxaline (Intermediate 4) (50.00 mg; 0.17 mmol; 1.00 eq.), (3S)-1-(pyrazin-2-yl)pyrrolidin-3-amine hydrochloride (Intermediate 104) (102.47 mg; 0.51 mmol; 3.00 eq.), NaOtBu (81.79 mg; 0.85 mmol; 5.00 eq.), BINAP (21.20 mg; 0.03 mmol; 0.20 eq.), Pd₂(dba)₃ (15.59 mg; 0.02 mmol; 0.10 eq.) and toluene (1.25 mL). Reaction is carried out in sealed tube at 120° C. for 24 h. Purification by FCC (DCM/MeOH; gradient). Repurification by preparative HPLC is done. 8-(1-methyl-1H-indol-6-yl)-N-[(3S)-1-(pyrazin-2-yl)pyrrolidin-3-yl]quinoxalin-6-amine (3.00 mg; yield 4.00%; 94.6% by HPLC) is obtained as a yellow powder.

### Intermediate 105

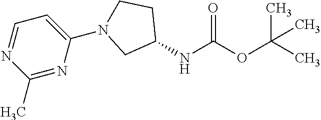 [SMILES: Cc1nccc(N2CC[C@H](NC(=O)OC(C)(C)C)C2)n1]

A sealed tube is charged with tert-butyl N-[(3S)-pyrrolidin-3-yl]carbamate (695.41 mg; 3.73 mmol; 1.2 eq.), 4-chloro-2-methylpyrimidine (400 mg; 3.11 mmol; 1.0 eq.), DIPEA (1.08 mL; 6.22 mmol; 2.0 eq.) and 1-BuOH (8.0 mL). RM is sealed and heated at 130° C. for 24h. After this time, the mixture is diluted with EtOAc, filtered through a Celite® pad. The filtrate is collected and evaporated to tert-butyl N-[(3S)-1-(2-methylpyrimidin-4-yl)pyrrolidin-3-yl]carbamate (403.40 mg; yield 46.6%; 100% by UPLC) as a beige solid.

### Intermediate 106

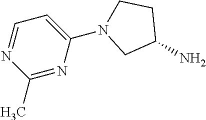

Roundbottom flask is charged with tert-butyl N-[(3S)-1-(2-methylpyrimidin-4-yl)pyrrolidin-3-yl]carbamate (Intermediate 105) (403.40 mg; 1.45 mmol; 1.0 eq.) and anhydrous DCM (20.17 mL). RM is cooled down to 0° C. and trifluoroacetic acid (0.58 mL; 7.25 mmol; 5.0 eq.) is added dropwise in this temperature. RM is stirred at room temperature for 24 h. After this time solvent is evaporated and crude product is dissolved in DCM. Organic solvent is washed with 2M NaOH_{(aq)}, water and brine. Organic solvent is evaporated to afford (3S)-1-(2-methylpyrimidin-4-yl)pyrrolidin-3-amine (Intermediate 104) (65.70 mg; yield 25.4%; 100% by UPLC) as a white solid.

### Example 164

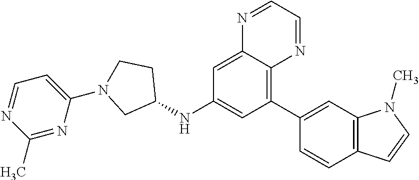 [SMILES: Cc1nccc(N2CC[C@H](Nc3cc(-c4ccc5ccn(C)c5c4)c4nccnc4c3)C2)n1]

The product is prepared according to General Procedure 2, described in Example 1 with 7-chloro-5-(1-methyl-1H-indol-6-yl)-quinoxaline (Intermediate 4) (50.00 mg; 0.17 mmol; 1.00 eq.), (3S)-1-(2-methylpyrimidin-4-yl)pyrrolidin-3-amine (Intermediate 106) (91.01 mg; 0.51 mmol; 3.00 eq.), NaOtBu (81.79 mg; 0.85 mmol; 5.00 eq.), BINAP (21.20 mg; 0.03 mmol; 0.20 eq.), Pd₂(dba)₃ (15.59 mg; 0.02 mmol; 0.10 eq.) and toluene (1.25 mL). Reaction is carried out in sealed tube at 120° C. for 24 h. Purification by FCC (DCM/MeOH; gradient). Repurification by preparative HPLC is done. 8-(1-methyl-1H-indol-6-yl)-N-[(3S)-1-(2-methylpyrimidin-4-yl)pyrrolidin-3-yl]quinoxalin-6-amine (23.30 mg; yield 30.0%; 95.5% by HPLC) is obtained as a green powder.

### Intermediate 107

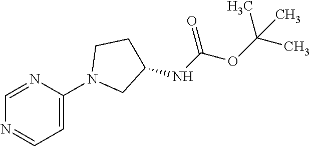 [SMILES: CC(C)(C)OC(=O)N[C@H]1CCN(c2ccncn2)C1]

Product is prepared according to procedure described in Example 164 for Intermediate 105 with N-[(3S)-pyrrolidin-3-yl]carbamate (325.0 mg; 1.75 mmol; 2.0 eq.), 4-chloropyrimidine (100 mg; 0.87 mmol; 1.0 eq.), DIPEA (0.3 mL; 6.22 mmol; 2.0 eq.) and 1-BuOH (2.0 mL). RM is stirred in a sealed tube at 16° C. for 4 h. Purification by FCC (MeOH/DCM, gradient). tert-Butyl N-[(3S)-1-(pyrimidin-4-yl)pyrrolidin-3-yl]carbamate (Intermediate 105) (186.0 mg; 0.70 mmol; yield 80.6%; 100% by UPLC) is obtained as colorless crystallizing oil.

### Intermediate 108

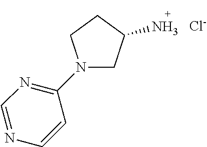 [SMILES: N[C@H]1CCN(c2ccncn2)C1 ; [Cl-]]

Product is prepared according to procedure described in Example 162 for Intermediate 102 with tert-butyl N-[(3S)-1-(pyrimidin-4-yl)pyrrolidin-3-yl]carbamate (Intermediate 107) (186.0 mg; 0.70 mmol; 1.0 eq), anhydrous Et₂O (5 mL) and 2M HCl in Et₂O (1.76 mL; 3.5 mmol; 5.0 eq.) RM is stirred at room temperature for 24 h and evaporated to give (3S)-1-(pyrimidin-4-yl)pyrrolidin-3-aminium chloride (156 mg; 0.66 mmol; yield 93.5%; 100% by UPLC) as gray solid.

### Example 165

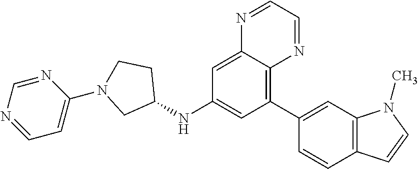 [SMILES: Cn1ccc2ccc(-c3cc(N[C@H]4CCN(c5ccncn5)C4)cc4nccnc34)cc21]

The product is prepared according to General Procedure 2, described in Example 1 with 7-chloro-5-(1-methyl-1H-indol-6-yl)-quinoxaline (Intermediate 4) (50.00 mg; 0.17 mmol; 1.00 eq.), (3S)-1-(pyrimidin-4-yl)pyrrolidin-3-aminium chloride (Intermediate 108) (80.6 mg; 0.34 mmol; 2.00 eq.), NaOtBu (98.0 mg; 1.02 mmol; 6.00 eq.), BINAP (21.20 mg; 0.03 mmol; 0.20 eq.), Pd₂(dba)₃ (15.6 mg; 0.02 mmol; 0.10 eq.) and [1,4]-dioxane (1.0 mL). Reaction is carried out in sealed tube at 120° C. for 16 h. Purification by FCC (DCM/MeOH; gradient). 8-(1-methyl-1H-indol-6-yl)-N-[(3S)-1-(pyrimidin-4-yl)-pyrrolidin-3-yl]quinoxalin-6-amine (72.8 mg; 0.17 mmol, yield 100%; 99.2% by HPLC) is obtained as a yellow foam.

### Intermediate 109

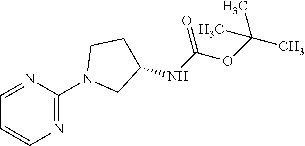 [SMILES: CC(C)(C)OC(=O)N[C@H]1CCN(c2ncccn2)C1]

Product is prepared according to procedure described in Example 164 for Intermediate 105 with N-[(3S)-pyrrolidin-3-yl]carbamate (325.0 mg; 1.75 mmol; 2.0 eq.), 2-chloropyrimidine (100 mg; 0.87 mmol; 1.0 eq.), DIPEA (0.3 mL; 6.22 mmol; 2.0 eq.) and 1-BuOH (2.0 mL). RM is stirred in a sealed tube at 160 C for 4 h. Purification by FCC (MeOH/DCM, gradient). tert-Butyl N-[(3S)-1-(pyrimidin-2-yl)pyrrolidin-3-yl]carbamate (169.0 mg; 0.64 mmol; yield 73.2%; 100% by UPLC) is obtained as white waxy solid.

### Intermediate 110

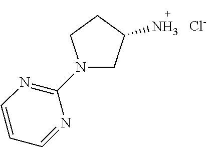 [SMILES: N[C@H]1CCN(c2ncccn2)C1 ; [Cl-]]

Product is prepared according to procedure described in Example 162 for Intermediate 102 with tert-butyl N-[(3S)-1-(pyrimidin-2-yl)pyrrolidin-3-yl]carbamate (Intermediate 109) (169.0 mg; 0.64 mmol; 1.0 eq), anhydrous Et₂O (5 mL) and 2M HCl in Et₂O (1.6 mL; 3.5 mmol; 5.0 eq.) RM is stirred at room temperature for 24 h and evaporated to give (3S)-1-(Pyrimidin-2-yl)pyrrolidin-3-aminium chloride (Intermediate 108) (146 mg; 0.66 mmol; yield 96.3%; 100% by UPLC) as gray solid.

### Example 166

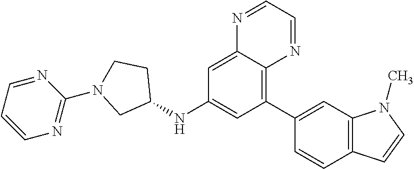 [SMILES: Cn1ccc2ccc(-c3cc(N[C@H]4CCN(c5ncccn5)C4)cc4nccnc34)cc21]

The product is prepared according to General Procedure 2, described in Example 1 with 7-chloro-5-(1-methyl-1H-indol-6-yl)-quinoxaline (Intermediate 4) (50.00 mg; 0.17 mmol; 1.00 eq.), (3S)-1-(Pyrimidin-2-yl)pyrrolidin-3-aminium chloride (Intermediate 110) (80.6 mg; 0.34 mmol; 2.00 eq.), NaOtBu (98.0 mg; 1.02 mmol; 6.00 eq.), BINAP (21.20 mg; 0.03 mmol; 0.20 eq.), Pd₂(dba)₃ (15.6 mg; 0.02 mmol; 0.10 eq.) and [1,4]-dioxane (1.0 mL). Reaction is carried out in sealed tube at 120° C. for 16 h. Purification by FCC (Hexane/EtOAc; gradient). 8-(1-Methyl-1H-indol-6-yl)-N-[(3S)-1-(pyrimidin-2-yl)pyrrolidin-3-yl]quinoxalin-6-amine (49.4 mg; 0.12 mmol, yield 68.7%; 99.6% by HPLC) is obtained as a neon yellow-green foam.

### Intermediate 111

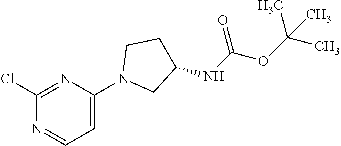 [SMILES: CC(C)(C)OC(=O)N[C@H]1CCN(c2ccnc(Cl)n2)C1]

Product is prepared according to procedure described in Example 164 for Intermediate 105 with N-[(3S)-pyrrolidin-3-yl]carbamate (150.0 mg; 0.81 mmol; 1.2 eq.), 2,4-dichloropyrimidine (100 mg; 0.67 mmol; 1.0 eq.), DIPEA (0.23 mL; 1.34 mmol; 2.0 eq.) and 1-BuOH (2.0 mL). RM is stirred in a sealed tube at 160 C for 4 h. Purification by FCC (MeOH/DCM, gradient). tert-Butyl N-[(3S)-1-(2-chloropyrimidin-4-yl)pyrrolidin-3-yl]carbamate (Intermediate 109) (142.0 mg; 0.49 mmol; yield 72.9%; 100% by UPLC) is obtained as colorless glossy oil.

### Intermediate 112

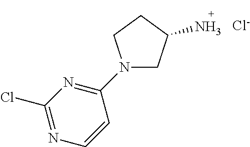 [SMILES: N[C@H]1CCN(c2ccnc(Cl)n2)C1 ; [Cl-]]

Product is prepared according to procedure described in Example 162 for Intermediate 102 with tert-butyl N-[(3S)-1-(2-chloropyrimidin-4-yl)pyrrolidin-3-yl]carbamate (Intermediate 111) (142.0 mg; 0.49 mmol; 1.0 eq), anhydrous Et₂O (5 mL) and 2M HCl in Et₂O (2.0 mL; 4.0 mmol; 8.2 eq.) RM is stirred at room temperature for 24 h and evaporated to give (3S)-1-(2-Chloropyrimidin-4-yl)pyrrolidin-3-aminium chloride (111 mg; 0.47 mmol; yield 96.5%; 100% by UPLC) as white powder.

### Example 167

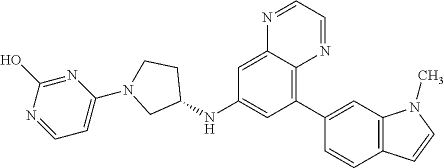 [SMILES: Cn1ccc2ccc(-c3cc(N[C@H]4CCN(c5ccnc(O)n5)C4)cc4nccnc34)cc21]

The product is prepared according to General Procedure 2, described in Example 1 with 7-chloro-5-(1-methyl-1H-indol-6-yl)-quinoxaline (Intermediate 4) (40.00 mg; 0.14 mmol; 1.00 eq.), (3S)-1-(2-Chloropyrimidin-4-yl)pyrrolidin-3-aminium chloride (Intermediate 112) (50 mg; 0.21 mmol; 1.56 eq.), NaOtBu (39.2 mg; 0.41 mmol; 3.00 eq.), BINAP (8.5 mg; 0.01 mmol; 0.10 eq.), Pd₂(dba)₃ (6.2 mg; 0.01 mmol; 0.05 eq.) and [1,4]-dioxane (2.0 mL). Reaction is carried out in sealed tube at 100° C. for 18 h. Purification by preparative HPLC (CAN/0.1% FA; gradient). 4-[(3S)-3-{[8-(1-Methyl-1H-indol-6-yl)quinoxalin-6-yl]amino}pyrrolidin-1-yl]pyrimidin-2-ol (13.2 mg; 0.03 mmol, yield 20.9%; 94.3% by HPLC) is obtained as a yellow powder.

### Intermediate 113

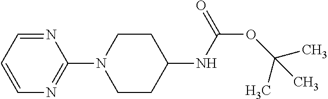 [SMILES: CC(C)(C)OC(=O)NC1CCN(c2ncccn2)CC1]

Product is prepared according to procedure described in Example 164 for Intermediate 105 with tert-butyl N-[piperidin-4-yl]carbamate (378.2 mg; 1.85 mmol; 2.0 eq.), 2-chloropyrimidine (106 mg; 0.93 mmol; 1.0 eq.), DIPEA (0.32 mL; 1.85 mmol; 2.0 eq.) and 1-BuOH (2.0 mL). RM is stirred in a sealed tube at 160 C for 4 h. Purification by FCC (MeOH/DCM, gradient). tert-Butyl N-[1-(pyrimidin-2-yl)piperidin-4-yl]carbamate (217.0 mg; 0.78 mmol; yield 84.2%; 98% by UPLC) is obtained as white flakes.

### Intermediate 114

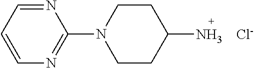 [SMILES: NC1CCN(c2ncccn2)CC1 ; [Cl-]]

Product is prepared according to procedure described in Example 162 for Intermediate 102 tert-butyl N-[1-(pyrimidin-2-yl)piperidin-4-yl]carbamate (Intermediate 113) (217.0 mg; 0.78 mmol; 1.0 eq), anhydrous Et₂O (5 mL) and 2M HCl in Et₂O (1.6 mL; 4.0 mmol; 4.0 eq.) RM is stirred at room temperature for 24 h and evaporated to give 1-(Pyrimidin-2-yl)piperidin-4-aminium chloride (199.4 mg; 0.78 mmol; yield 99.8%; 98% by UPLC) as beige powder.

### Example 168

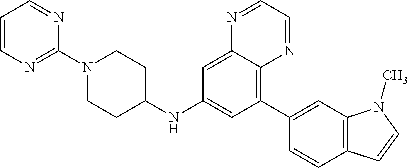 [SMILES: Cn1ccc2ccc(-c3cc(NC4CCN(c5ncccn5)CC4)cc4nccnc34)cc21]

The product is prepared according to General Procedure 2, described in Example 1 with 7-chloro-5-(1-methyl-1H-indol-6-yl)-quinoxaline (Intermediate 4) (50.00 mg; 0.17 mmol; 1.00 eq.), (1-(Pyrimidin-2-yl)piperidin-4-aminium chloride (Intermediate 114) (52.2 mg; 0.2 mmol; 1.2 eq.), NaOtBu (65.3 g; 0.68 mmol; 4.0 eq.), BINAP (10.6 mg; 0.02 mmol; 0.10 eq.), Pd₂(dba)₃ (7.8 mg; 0.01 mmol; 0.05 eq.) and [1,4]-dioxane (1.0 mL). Reaction is carried out in sealed tube at 120° C. for 16 h. Purification by FCC (MeOH/EtOAc; gradient). 8-(1-Methyl-1H-indol-6-yl)-N-[1-(pyrimidin-2-yl)piperidin-4-yl]quinoxalin-6-amine (33.5 mg; 0.08 mmol, yield 45.1%; 99.7% by HPLC) is obtained as a neon greenish-yellow foam.

### Example 169

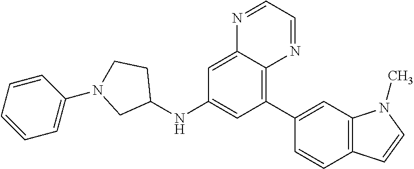 [SMILES: Cn1ccc2ccc(-c3cc(NC4CCN(c5ccccc5)C4)cc4nccnc34)cc21]

The product is prepared according to General Procedure 2, described in Example 1 with 7-chloro-5-(1-methyl-1H-indol-6-yl)-quinoxaline (Intermediate 4) (40.00 mg; 0.14 mmol; 1.00 eq.), 1-phenylpyrrolidin-3-amine (55.67 mg; 0.34 mmol; 2.52 eq.), NaOtBu (52.35 mg; 0.54 mmol; 4.00 eq.), BINAP (16.96 mg; 0.03 mmol; 0.20 eq.), Pd₂(dba)₃ (12.47 mg; 0.01 mmol; 0.10 eq.) and toluene (1.20 mL). Reaction is carried out in sealed tube at 120° C. for 24 h. Purification by FCC (DCM/MeOH; gradient). 8-(1-methyl-1H-indol-6-yl)-N-(1-phenylpyrrolidin-3-yl)quinoxalin-6-amine (54.00 mg; yield 90.3%; 95.5% by HPLC) is obtained as a yellow powder.

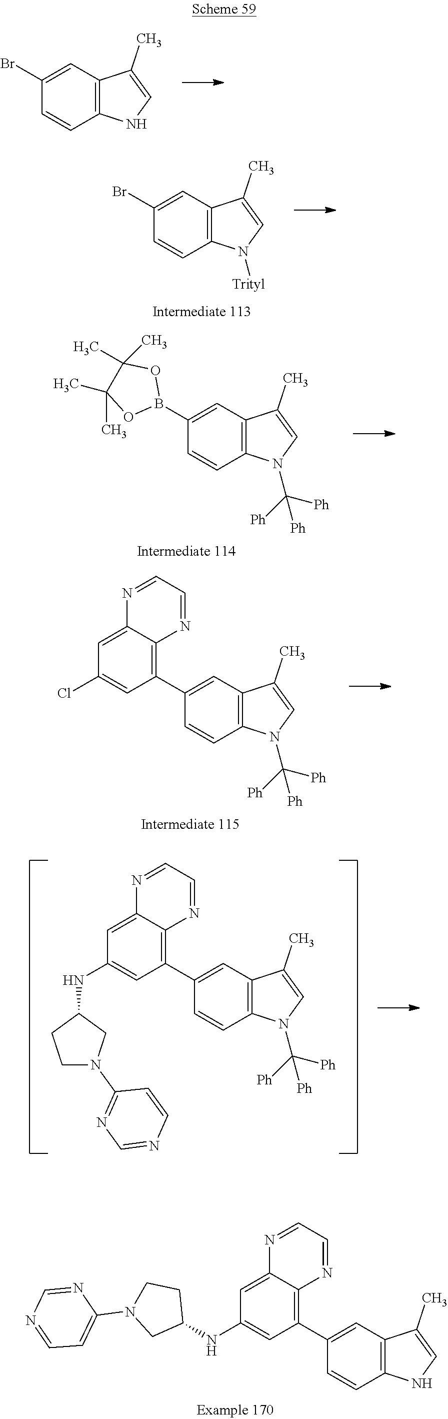

### Intermediate 115

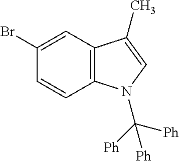 [SMILES: Cc1cn(C(c2ccccc2)(c2ccccc2)c2ccccc2)c2ccc(Br)cc12]

Roundbottom flask is charged with 5-bromo-3-methyl-1H-indole (330 mg; 1.57 mmol; 1.00 eq.) and anhydrous THF (5 mL). The NaH (126 mg; 3.14 mmol; 2.00 eq.) is added in small portions over 5 min to the stirred RM. When gas evolution is stopped the Trityl-Cl (876 mg; 3.14 mmol; 2.00 eq.) is added in one portion. Resulted mixture is stirred for 18 h, and RM is quenched by MeOH (2 mL) addition. Resulted slurry is evaporated in vacuo. Resulted residue is purified by FCC (Hexane/EtOAc, gradient). 5-Bromo-3-methyl-1-(triphenylmethyl)-1H-indole (Intermediate 115) (180.0 mg; 0.33 mmol; yield 20.8%; 82% by UPLC) is obtained as light brown solid.

### Intermediate 116

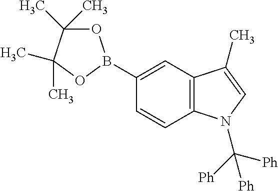 [SMILES: Cc1cn(C(c2ccccc2)(c2ccccc2)c2ccccc2)c2ccc(B3OC(C)(C)C(C)(C)O3)cc12]

Product is prepared according to General Procedure 51, described for Intermediate 81 with 5-bromo-3-methyl-1-(triphenylmethyl)-1H-indole (Intermediate 115) (180.0 mg; 0.33 mmol; 1.00 eq.), 4,4,5,5-tetramethyl-2-(tetramethyl-1,3,2-dioxaborolan-2-yl)-1,3,2-dioxaborolane (108 mg; 0.42 mmol; 1.30 eq.), KOAc (64 mg; 0.65 mmol; 2.00 eq.), Pd(dppf)Cl₂ (24 mg; 0.03 mmol; 0.1 eq.) and [1,4]-dioxane (5.00 ml). Reaction is carried out for 18 h at 100° C. Purification by FCC (hexane/EtOAc; gradient). 3-methyl-5-(tetramethyl-1,3,2-dioxaborolan-2-yl)-1-(triphenylmethyl)-1H-indole (138.3 mg; 0.24 mmol; Yield 74.7%; 88% by UPLC) is obtained as colorless oil.

### Intermediate 117—General Procedure 54

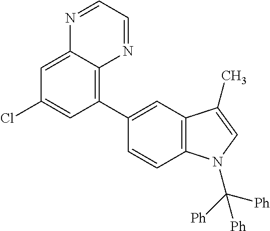 [SMILES: Cc1cn(C(c2ccccc2)(c2ccccc2)c2ccccc2)c2ccc(-c3cc(Cl)cc4nccnc34)cc12]

The product is prepared according to modified General Procedure 1, described for Intermediate 4 with 5-bromo-7-chloroquinoxaline (Intermediate 2) (50 mg; 0.20 mmol; 1.00 eq.), 3-methyl-5-(tetramethyl-1,3,2-dioxaborolan-2-yl)-1-(triphenylmethyl)-1H-indole (Intermediate 116) (13 mg; 0.23 mmol; 1.13 eq.), DIPEA (0.07 ml; 0.04 mmol; 2.00 eq.), Pd(dppf)Cl₂ (14.7 mg; 0.02 mmol; 0.10 eq.), water (0.3 ml) and [1,4]-dioxane (1.0 ml). RM is heated to 120° C. and irradiated with MW in the Biotage Initiator unit for 30 min. Purification by FCC (Hexane/EtOAc, gradient). 7-Chloro-5-(1-ethyl-1H-indol-6-yl)quinoxaline (Intermediate 117) (19.0 mg; 0.03 mmol; yield 16%; 93% by UPLC) is obtained as yellow foam.

### Example 170

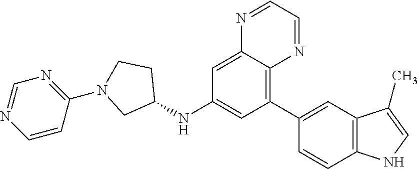 [SMILES: Cc1c[nH]c2ccc(-c3cc(N[C@H]4CCN(c5ccncn5)C4)cc4nccnc34)cc12]

The product is prepared according to General Procedure 2, described in Example 1 with 7-Chloro-5-(1-ethyl-1H-indol-6-yl)quinoxaline (Intermediate 117) (19.0 mg; 0.03 mmol 1.00 eq.), (3S)-1-(pyrimidin-4-yl)pyrrolidin-3-aminium chloride (Intermediate 108) (15.6 mg; 0.07 mmol; 20 eq.), NaOtBu (16 mg; 0.16 mmol; 5.0 eq.), BINAP (2.0 mg; 0.01 mmol; 0.10 eq.), Pd₂(dba)₃ (1.5 mg; 0.01 mmol; 0.05 eq.) and [1,4]-dioxane (0.5 mL). Reaction is carried out in sealed tube at 110° C. for 18 h. Purification by FCC (MeOH/EtOAc; gradient). 8-(1-Methyl-1H-indol-6-yl)-N-[1-(pyrimidin-2-yl)piperidin-4-yl]quinoxalin-6-amine (12.1 mg; 0.03 mmol; yield 81.7%; 93.8% by HPLC) is obtained as a yellow-green solid.

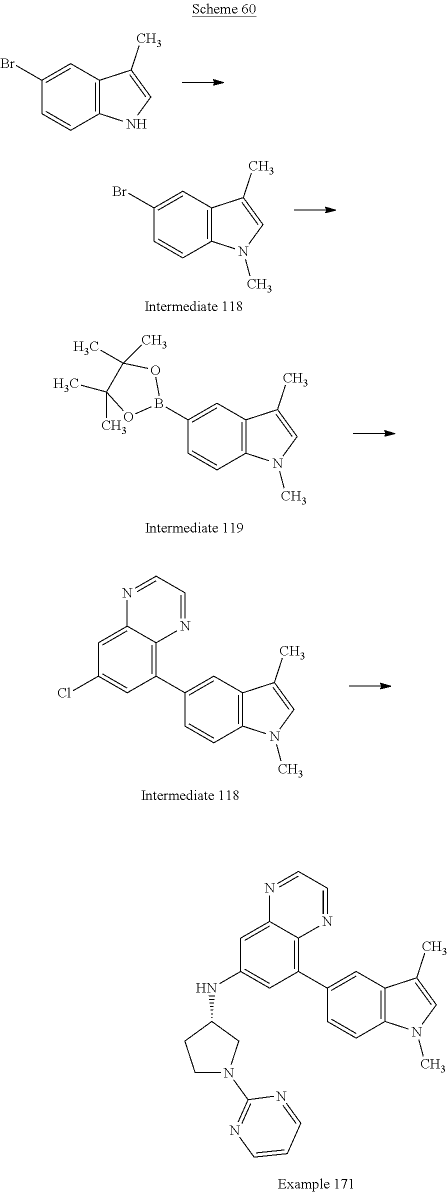 [SMILES: Cc1c[nH]c2ccc(Br)cc12 ; Cc1cn(C)c2ccc(Br)cc12 ; Cc1cn(C)c2ccc(B3OC(C)(C)C(C)(C)O3)cc12 ; Cc1cn(C)c2ccc(-c3cc(Cl)cc4nccnc34)cc12 ; Cc1cn(C)c2ccc(-c3cc(N[C@H]4CCN(c5ncccn5)C4)cc4nccnc34)cc12]

### Intermediate 118

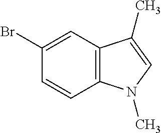

Product is prepared according to procedure described in literature (Fraile, J. M.; Le Jeune, K.; Mayoral, J. A.; Ravasio, N.; Zaccheria, F.; Org. Biomol. Chem. 2013, v:11, pp: 4327-4332). A solution of 5-bromo-3-methyl-1H-indole (0.30 g; 1.437 mmol; 1.00 eq.) in dry THF (5.0 ml), is cooled to 0-5° C. then NaH (60% immersion in mineral oil) (0.14 g; 2.86 mmol; 2.00 eq.) is added in small portions over 10 min. RM is stirred for 1h then Iodoethane (0.21 ml; 2.86 mmol; 2.0 eq.) is added dropwise. Reaction mixture is stirred for next 30 min at 0° C. and 18 h at rt. After mentioned time RM is poured onto ice and extracted with diethyl ether. Organic layers is washed with brine and dried over Na₂SO₄. Solvent is evaporated in vacuo to provide desired product 5-bromo-1,3-dimethyl-1H-indole (0.355 g; 1.33 mmol; yield 93.2%; 84% by UPLC) is obtained as light yellow oil.

### Intermediate 119

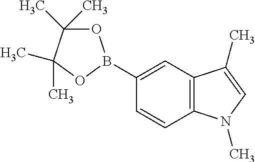 [SMILES: Cc1cn(C)c2ccc(B3OC(C)(C)C(C)(C)O3)cc12]

Product is prepared according to General Procedure 51, described for Intermediate 81 with 5-bromo-1,3-dimethyl-1H-indole (Intermediate 118) (0.355 g; 1.33 mmol; 1.00 eq.), 4,4,5,5-tetramethyl-2-(tetramethyl-1,3,2-dioxaborolan-2-yl)-1,3,2-dioxaborolane (439.5 mg; 1.73 mmol; 1.30 eq.), KOAc (261 mg; 2.66 mmol; 2.00 eq.), Pd(dppf)Cl₂ (97.4 mg; 0.13 mmol; 0.1 eq.) and [1,4]-dioxane (5.00 ml). Reaction is carried out for 18 h at 100° C. Purification by FCC (hexane/EtOAc; gradient). 1,3-dimethyl-5-(tetramethyl-1,3,2-dioxaborolan-2-yl)-1H-indole (Intermediate 119) (282 mg; 1.01 mmol; Yield 75.8%; 97% by UPLC) is obtained as colorless oil.

### Intermediate 120

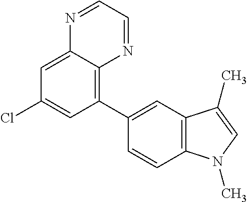 [SMILES: Cc1cn(C)c2ccc(-c3cc(Cl)cc4nccnc34)cc12]

The product is prepared according to modified General procedure for Suzuki-Miyaura cross couplings under MW conditions, described for Intermediate 154 with 5-bromo-7-chloroquinoxaline (Intermediate 2) (100 mg; 0.40 mmol; 1.00 eq.), 1,3-dimethyl-5-(tetramethyl-1,3,2-dioxaborolan-2-yl)-1H-indole (Intermediate 119) (142 mg; 0.52 mmol, 1.3 eq) DIPEA (0.14 ml; 0.08 mmol; 2.00 eq.), Pd(dppf)Cl₂ (29.5 mg; 0.04 mmol; 0.10 eq.), water (1.0 ml) and [1,4]-dioxane (3.0 ml). RM is heated to 120° C. and irradiated with MW in the Biotage Initiator unit for 30 min. Purification by FCC (Hexane/EtOAc, gradient). 7-Chloro-5-(1,3-dimethyl-1H-indol-5-yl)quinoxaline (Intermediate 120) (78.0 mg; 0.25 mmol; yield 62.8%; 100% by UPLC) is obtained as yellow solid.

### Example 171

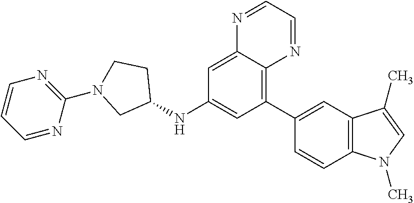 [SMILES: Cc1cn(C)c2ccc(-c3cc(N[C@H]4CCN(c5ncccn5)C4)cc4nccnc34)cc12]

The product is prepared according to General Procedure 2, described in Example 1 with 7-chloro-5-(1,3-dimethyl-1H-indol-5-yl)quinoxaline (Intermediate 120) (39.0 mg; 0.13 mmol 1.00 eq.), (3S)-1-(Pyrimidin-2-yl)pyrrolidin-3-aminium chloride (Intermediate 110) (45.1 mg; 0.19 mmol; 1.50 eq.), NaOtBu (48.7 mg; 0.51 mmol; 4.0 eq.), BINAP (7.9 mg; 0.01 mmol; 0.10 eq.), Pd₂(dba)₃ (5.8 mg; 0.01 mmol; 0.05 eq.) and [1,4]-dioxane (1.0 mL). Reaction is carried out in sealed tube at 110° C. for 18 h. Purification by FCC (Hexane/EtOAc; gradient). 8-(1,3-Dimethyl-1H-indol-5-yl)-N-[(3S)-1-(pyrimidin-2-yl)pyrrolidin-3-yl]quinoxalin-6-amine (42.9 mg; 0.10 mmol; yield 77.7%; 99.5% by HPLC) is obtained as a yellow solid.

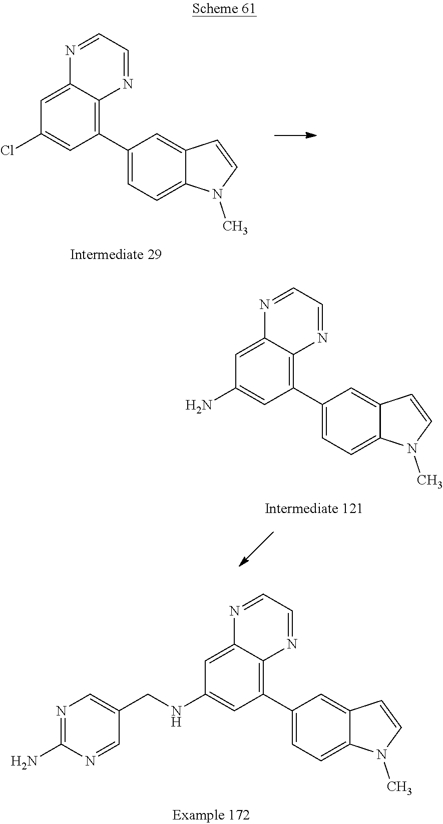 [SMILES: Cn1ccc2cc(-c3cc(Cl)cc4nccnc34)ccc21 ; Cn1ccc2cc(-c3cc(N)cc4nccnc34)ccc21 ; Cn1ccc2cc(-c3cc(NCc4cnc(N)nc4)cc4nccnc34)ccc21]

### Intermediate 121

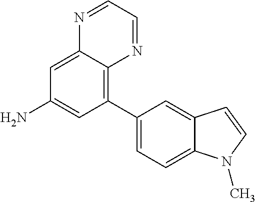 [SMILES: Cn1ccc2cc(-c3cc(N)cc4nccnc34)ccc21]

The product is prepared according to General Procedure 19, described for Intermediate 22 with 7-chloro-5-(1-methyl-1H-indol-5-yl)quinoxaline (Intermediate 29) (500.00 mg; 1.67 mmol; 1.00 eq.), ammonia (0.5 M in 1,4-dioxane) (50.04 ml; 25.02 mmol; 15.00 eq.), Pd₂(dba)₃ (112.55 mg; 0.12 mmol; 0.07 eq.), Me₄tBuXPhos (57.28 mg; 0.12 mmol; 0.07 eq.) and NaOtBu (224.43 mg; 2.34 mmol; 1.40 eq.). Reaction is carried out for 5 h at 80° C. Then it is filtered through Celite® and the filtrate is evaporated under reduced pressure. Purification by FCC (DCM/AcOEt; gradient) to give 8-(1-methyl-1H-indol-5-yl)quinoxalin-6-amine (405.00 mg; yield 82.7%; 93.4% by UPLC) as a yellow powder.

### Example 172

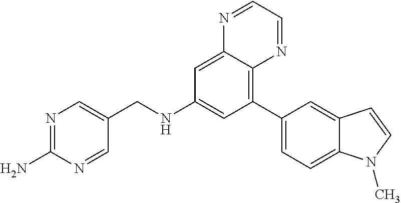 [SMILES: Cn1ccc2cc(-c3cc(NCc4cnc(N)nc4)cc4nccnc34)ccc21]

The product is prepared according to General Procedure 23, described in Example 63 with 8-(1-methyl-1H-indol-5-yl)quinoxalin-6-amine (Intermediate 121) (100.00 mg; 0.34 mmol; 1.00 eq.), 2-amino-pyrimidine-5-carbaldehyde (69.09 mg; 0.53 mmol; 1.57 eq.), DCE (16.00 ml), Hantzsch ester (355.36 mg; 1.32 mmol; 3.9 eq.) and TMCS (0.111 ml; 0.87 mmol; 2.52 eq.) (added in portions during heating up). Reaction is carried out for 6 h at RT then RM is heated additionally at 55° C. until no further substantial progress of the reaction is observed. Purification by FCC (DCM/MeOH; gradient) to give N-[(2-aminopyrimidin-5-yl)methyl]-8-(1-methyl-1H-indol-5-yl)quinoxalin-6-amine (9.50 mg; yield 7.2%; 98.4% by HPLC) as a yellow solid.

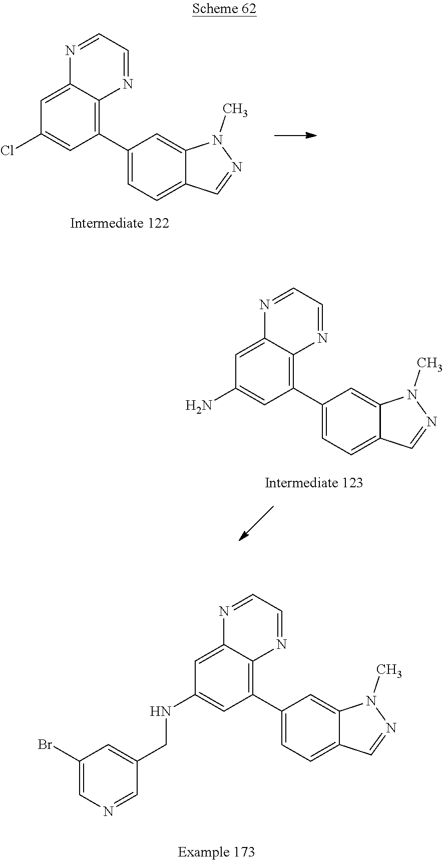 [SMILES: Cn1ncc2ccc(-c3cc(Cl)cc4nccnc34)cc21 ; Cn1ncc2ccc(-c3cc(N)cc4nccnc34)cc21 ; Cn1ncc2ccc(-c3cc(NCc4cncc(Br)c4)cc4nccnc34)cc21]

### Intermediate 122

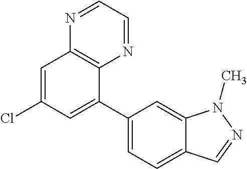 [SMILES: Cn1ncc2ccc(-c3cc(Cl)cc4nccnc34)cc21]

The product is prepared according to General Procedure 1, described for Intermediate 4 with 5-bromo-7-chloroquinoxaline (Intermediate 2) (276.72 mg; 1.14 mmol; 1.00 eq.), 1-methyl-6-(tetramethyl-1,3,2-dioxaborolan-2-yl)-1H-indazole (200 mg; 1.14 mmol; 1.00 eq.), DIPEA (0.40 mL; 2.27 mmol; 2.00 eq.), Pd(dppf)Cl₂ (83.12 mg; 0.11 mmol; 0.10 eq.), 1,4-dioxane (2.50 mL) and water (2.50 mL). Purification by FCC (hexane/EtOAc; gradient). 7-Chloro-5-(1-methyl-1H-indazol-6-yl)quinoxaline (Intermediate 122) (150 mg; yield 25.1%; 56% by HPLC) is obtained as a yellow crystals.

### Intermediate 123

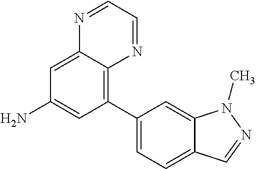 [SMILES: Cn1ncc2ccc(-c3cc(N)cc4nccnc34)cc21]

The product is prepared according to General Procedure 19, described for Intermediate 122 with 7-chloro-5-(1-methyl-1H-indazol-6-yl)-quinoxaline (Intermediate 122) (93.00 mg; 0.27 mmol; 1.00 eq.), Pd₂(dba)₃ (18.18 mg; 0.02 mmol; 0.07 eq.), Me₄tBuXPhos (9.25 mg; 0.02 mmol; 0.07 eq.), ammonia (0.5 M in 1,4-dioxane) (8.08 ml; 4.04 mmol; 15.00 eq.) and NaOtBu (36.26 mg; 0.38 mmol; 1.40 eq.). Reaction is carried out for 5 h at 80° C. with stirring. Purification by FCC (DCM/MeOH; gradient) to give 8-(1-methyl-1H-indazol-6-yl)quinoxalin-6-amine (35.00 mg; yield 46.5%; 98.6% by UPLC) as a yellow film.

### Example 173

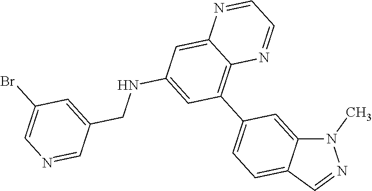 [SMILES: Cn1ncc2ccc(-c3cc(NCc4cncc(Br)c4)cc4nccnc34)cc21]

The product is prepared according to General Procedure 23, described in Example 63 with 8-(1-methyl-1H-indazol-6-yl)quinoxalin-6-amine (Intermediate 123) (28.00 mg; 0.10 mmol; 1.00 eq.), 5-bromopyridine-3-carbaldehyde (38.00 mg; 0.20 mmol; 2.04 eq.), Hantzsch ester (31.00 mg; 0.12 mmol; 1.22 eq.), TCMS (10.00 μl; 0.08 mmol; 0.79 eq.) and DCM (8.00 ml)/DCE (2.00 ml) mixture. Reaction is carried out for 30 h at RT. Purification by FCC (DCM/MeOH; gradient) to give N-[(5-bromopyridin-3-yl)methyl]-8-(1-methyl-1H-indazol-6-yl)quinoxalin-6-amine (19.80 mg; yield 43.4%; 97.8% by HPLC) as a brown orange solid.

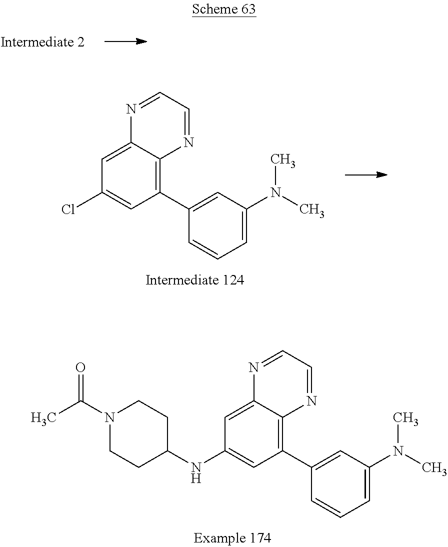

### Intermediate 124

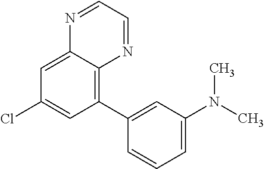 [SMILES: CN(C)c1cccc(-c2cc(Cl)cc3nccnc23)c1]

Product is prepared according to General Procedure 1, described for Intermediate 4 with (0.300 g; 1.23 mmol; 1.00 eq.), [3-(dimethylamino)-phenyl]boronic acid (0.224 g; 1.36 mmol; 1.10 eq.), DIPEA (0.43 ml; 2.46 mmol; 2.00 eq.), Pd(dppf)Cl₂ (90 mg; 0.12 mmol; 0.10 eq.), [1,4]-dioxane (3.00 ml) and water (3.00 ml). Reaction is carried out for 18 h at 85° C. Purification by FCC (hexane/EtOAc, gradient). 3-(7-Chloroquinoxalin-5-yl)-N,N-dimethylaniline (158.00 mg; 0.55 mmol; yield 44.8%; 00% by UPLC) is obtained as yellow flakes.

### Example 174

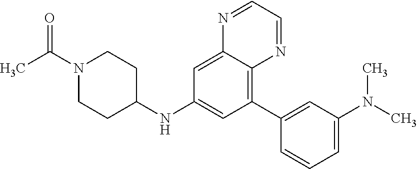 [SMILES: CC(=O)N1CCC(Nc2cc(-c3cccc(N(C)C)c3)c3nccnc3c2)CC1]

Product is prepared according to General Procedure 2, described in Example 1 with 3-(7Chloroquinoxalin-5-yl)-N,N-dimethylaniline (Intermediate 124) (50.00 mg; 0.16 mmol; 1.00 eq.), 1-(4-aminopiperidin-1-yl)ethan-1-one hydrochloride (50 mg; 0.28 mmol; 2.00 eq.), NaOtBu (54 mg; 0.56 mmol; 4.00 eq.), BINAP (18 mg; 0.03 mmol; 0.20 eq.), Pd₂(dba)₃ (13 mg; 0.02 mmol; 0.10 eq.) and Toluene (3.00 ml). Reaction is carried out for 18 h at 120° C. Purification by FCC (MeOH/DCM, gradient). 1-[4-({8-[3-(Dimethyl-amino)phenyl]quinoxalin-6-yl}amino)piperidin-1-yl]ethan-1-one (20.40 mg; 0.05 mmol; yield 36.7%; 97.9% by HPLC) is obtained as brownish-yellow solid.

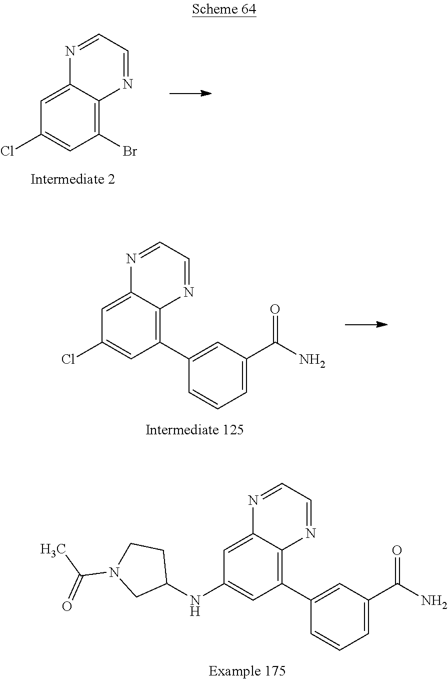 [SMILES: Clc1cc(Br)c2nccnc2c1 ; NC(=O)c1cccc(-c2cc(Cl)cc3nccnc23)c1 ; CC(=O)N1CCC(Nc2cc(-c3cccc(C(N)=O)c3)c3nccnc3c2)C1]

### Intermediate 125

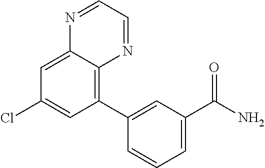 [SMILES: NC(=O)c1cccc(-c2cc(Cl)cc3nccnc23)c1]

The product is prepared according to modified General procedure for Suzuki-Miyaura cross couplings under MW conditions, described for Intermediate 154 with 5-bromo-7-chloroquinoxaline (Intermediate 2) (0.25 g; 1.21 mmol; 1.00 eq.), 3-(tetramethyl-1,3,2-dioxaborolan-2-yl)benzamide (299 mg; 1.21 mmol; 1.2 eq.), cesium carbonate (730.77 mg; 2.22 mmol; 2.20 eq.), Pd(dppf)Cl₂ (37 mg; 0.05 mmol; 0.05 eq.), 1,4-dioxane (2.5 mL) and water (0.8 mL). RM is heated to 120° C. and irradiated with MW in the Biotage Initiator unit for 30 min. RM is diluted with EtOAc and filtered by Celite® pad. Filtrate is extracted with water and brine, dried over Na₂SO₄. Drying agent is filtered off and solvent evaporated to give crude 3-(7-chloroquinoxalin-5-yl)benzamide (Intermediate 125) (278 mg; 0.83 mmol, yield 83%; 85% by UPLC) as a dark solid.

### Example 175

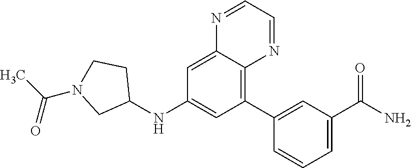 [SMILES: CC(=O)N1CCC(Nc2cc(-c3cccc(C(N)=O)c3)c3nccnc3c2)C1]

The product is prepared according to modified General Procedure 2, described in Example 1 with 3-(7-chloroquinoxalin-5-yl)benzamide (Intermediate 125) (70.00 mg; 0.21 mmol; 1.00 eq.), 1-(3-aminopyrrolidin-1-yl)-ethan-1-one (32.3 mg; 0.25 mmol; 1.2 eq.), NaOtBu (60 mg; 0.63 mmol; 3.00 eq.), BINAP (26 mg; 0.04 mmol; 0.20 eq.), Pd₂(dba)₃ (19.2 mg; 0.02 mmol; 0.10 eq.) and [1,4]-dioxane (1.40 mL). Reaction is carried out in sealed tube at 120° C. for 18 h. Purification by FCC (DCM/MeOH; gradient) gives 15.3 mg (80% by UPLC) crude product. 3-{7-[(1-Acetylpyrrolidin-3-yl)amino]quinoxalin-5-yl}benzamide (11.3 mg; 0.03 mmol; yield 14.3%; 99.8% by HPLC) is obtained after repurification by prep HPLC (ACN/0.05% formic acid, gradient) as a bright yellow solid.

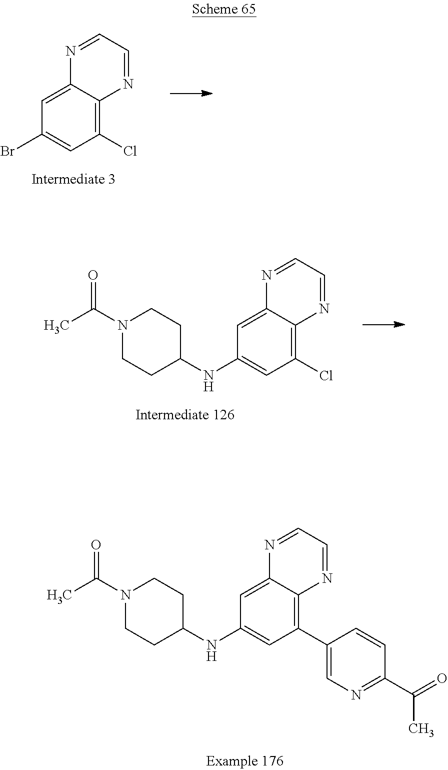 [SMILES: Clc1cc(Br)cc2nccnc12 ; CC(=O)N1CCC(Nc2cc(Cl)c3nccnc3c2)CC1 ; CC(=O)c1ccc(-c2cc(NC3CCN(C(C)=O)CC3)cc3nccnc23)cn1]

### Intermediate 126

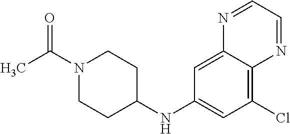 [SMILES: CC(=O)N1CCC(Nc2cc(Cl)c3nccnc3c2)CC1]

The sealed tube is charged with 7-bromo-5-chloroquinoxaline (50 mg; 0.21 mmol; 1.00 eq.), 1-(4-aminopiperidin-1-yl)ethan-1-one hydrochloride (38 mg; 0.27 mmol; 1.30 eq.), NaOtBu (59 mg; 0.62 mmol; 3.00 eq.), BrettPhos Pd G1 (3.3 mg; 0.00 mmol; 0.02 eq.), BrettPhos (4.4 mg; 0.01 mmol; 0.04 eq.) and sealed with silicone PTFE coated cap. The air from the vessel is evacuated in vacuo thorough syringe and backfilled with argon. The cycle is repeated 3 times and anhydrous [1,4]-dioxane (1.00 ml) is added thorough syringe. RM is heated and stirred for 1 h at 120° C. Then RM is diluted with EtOAc and filtered through Celite® pad. Filtrate is evaporated resulted oily residue is purified by FCC (MeOH/DCM, gradient). 1-{4-[(8-Chloroquinoxalin-6-yl)amino]piperidin-1-yl}ethan-1-one (6.9 mg; 0.02 mmol; yield 10.4%; 94% by UPLC) is obtained as brown glass.

### Example 176

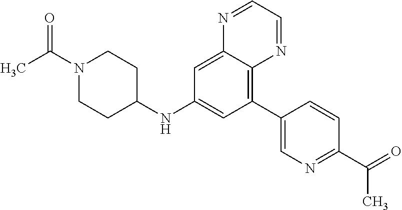 [SMILES: CC(=O)c1ccc(-c2cc(NC3CCN(C(C)=O)CC3)cc3nccnc23)cn1]

The MW reacting vessel is charged with 1-{4-[(8-Chloroquinoxalin-6-yl)amino]piperidin-1-yl}ethan-1-one (Intermediate 126) (6.90 mg; 0.02 mmol; 1.00 eq.), 1-[5-(tetramethyl-1,3,2-dioxaborolan-2-yl)pyridin-2-yl]ethan-1-one (13.5 mg; 0.05 mmol; 2.50 eq.), cessium carbonate (21 mg; 0.06 mmol; 3.00 eq.), water (0.02 ml), [1,4]-dioxane (0.07 ml). Resulted slurry is flushed with argon and and XPhos Pd G3 (0.90 mg; 0.00 mmol; 0.05 eq.) is added under argon. The vessel is capped and the RM is heated to 120° C. and irradiated with MW in the Biotage Initiator unit for 60 min. RM is diluted with EtOAc and filtrated through Celite® pad. Filtrate is extracted with water and brine. Organic layer is dried over Na₂SO₄ and evaporated. Purification by FCC (MeOH/DCM, gradient). 1-(5-{7-[(1-Acetylpiperidin-4-yl)amino]quinoxalin-5-yl}pyridin-2-yl)ethan-1-one (4.10 mg; 0.01 mmol; yield 46.2%; 93.3% by HPLC) is obtained as yellow-brownish glass.

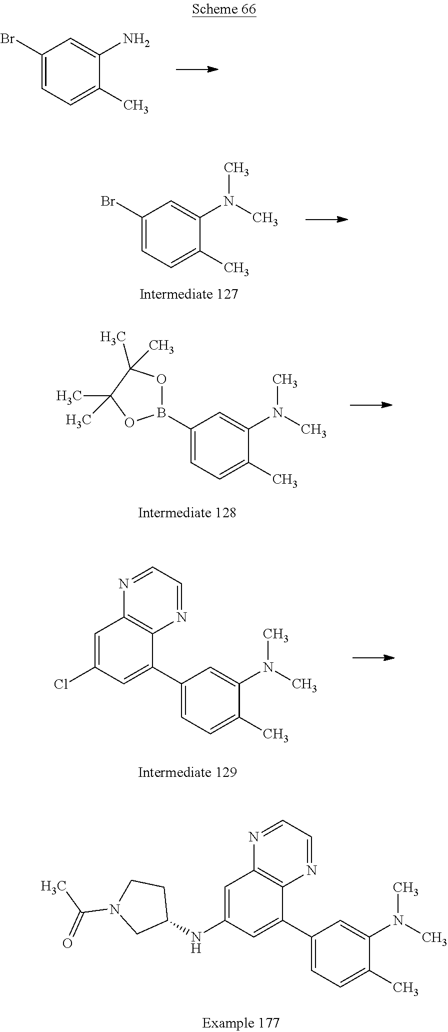

### Intermediate 127

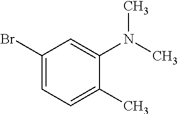

Roundbottom flask is charged with 5-bromo-2-methylaniline (100.00 mg; 0.54 mmol; 1.0 eq), methanol (1.5 mL), 37% formaldehyde solution in methanol (289.18 mg; 3.56 mmol; 2.40 eq.) and acetic acid (104.68 mg; 1.29 mmol; 2.40 eq.). RM is stirred at room temperature for 10 minutes. After this time RM is cooled down to 00° C. and sodiumcyanoborohydride (70.93 mg; 1.13 mmol; 2.10 eq.) is added. Reaction mixture is stirred in this temperature for 1 h. Then the solvent is evaporated and reaction is quenched with saturated aqueous solution of NaHCO3 (5 mL) and extracted with DCM (3×5 mL). Combined organic extracts are washed with water (2×10 mL). Organic solvent is then evaporated and crude product is further purified by FCC (DCM/EtOAc, gradient) to 5-bromo-N,N,2-trimethylaniline (74.20 mg; yield 61.6%; 95.5% by UPLC) as a colorless oil.

### Intermediate 128

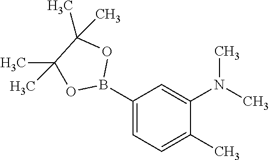

Product is obtained according to general procedure 51 described for Intermediate 81. A sealed tube is charged with 5-bromo-N,N,2-trimethylaniline (Intermediate 127) (74.50 mg; 0.34 mmol; 1.0 eq.), 4,4,5,5-tetramethyl-2-(tetramethyl-1,3,2-dioxaborolan-2-yl)-1,3,2-dioxaborolane (113.72 mg; 0.45 mmol; 1.3 eq.), KOAc (67.62 mg; 0.69 mmol; 2.0 eq.) and dioxane (0.75 mL), Then RM is purged with argon and then Pd(dppf)Cl2 (25.21 mg; 0.03 mmol; 0.10 eq.) is added. RM is sealed and heated at 100° C. for 18h. After this time, the mixture is diluted with EtOAc, filtered through a Celite® pad. The filtrate is collected and evaporated. Crude product is purified by FCC (hexane/EtOAc; gradient) N,N-2-trimethyl-5-(tetramethyl-1,3,2-dioxaborolan-2-yl)aniline (Intermediate 126)(42.50 mg; yield 47.2%; 100% by HPLC) is obtained as an oil.

### Intermediate 129

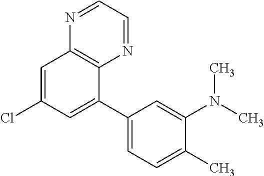 [SMILES: Cc1ccc(-c2cc(Cl)cc3nccnc23)cc1N(C)C]

The product is prepared according to General Procedure 1, described for Intermediate 4 with 5-bromo-7-chloroquinoxaline (Intermediate 2) (39.62 mg; 0.16 mmol; 1.00 eq.), N,N,2-trimethyl-5-(tetramethyl-1,3,2-dioxaborolan-2-yl)aniline (Intermediate 128) (42.50 mg; 0.16 mmol; 1.00 eq.), DIPEA (0.06 mL; 0.33 mmol; 2.00 eq.), Pd(dppf)Cl₂ (11.90 mg; 0.02 mmol; 0.10 eq.), 1,4-dioxane (2.50 mL) and water (2.50 mL). Purification by FCC (hexane/EtOAc; gradient). 5-(7-chloroquinoxalin-5-yl)-N,N,2-trimethylaniline (Intermediate 127) (23.90 mg; yield 49.1%; 99.5% by HPLC) is obtained as a yellow crystals.

### Example 177

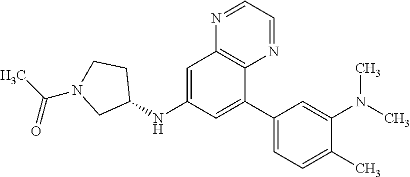 [SMILES: CC(=O)N1CC[C@H](Nc2cc(-c3ccc(C)c(N(C)C)c3)c3nccnc3c2)C1]

The product is prepared according to General Procedure 2, described in Example 1 with 5-(7-chloroquinoxalin-5-yl)-N,N,2-trimethylaniline (Intermediate 129) (23.90 mg; 0.14 mmol; 1.00 eq.), 1-[(3S)-3-aminopyrrolidin-1-yl]ethan-1-one hydrochloride (30.86 mg; 0.34 mmol; 3.00 eq.), NaOtBu (30.85 mg; 0.32 mmol; 4.00 eq.), BINAP (10.00 mg; 0.02 mmol; 0.20 eq.), Pd₂(dba)₃ (7.35 mg; 0.01 mmol; 0.10 eq.) and toluene (1.00 mL). Reaction is carried out in a well-sealed tube with silicone PTFE coated cap at 120° C. for 24 h. Purification by FCC (DCM/MeOH; gradient). 1-[(3S)-3-({8-[3-(dimethyl-amino)-4-methylphenyl]quinoxalin-6-yl}amino)pyrrolidin-1-yl]ethan-1-one (29.60 mg; yield 88.4%; 93.4% by HPLC) is obtained as a beige powder.

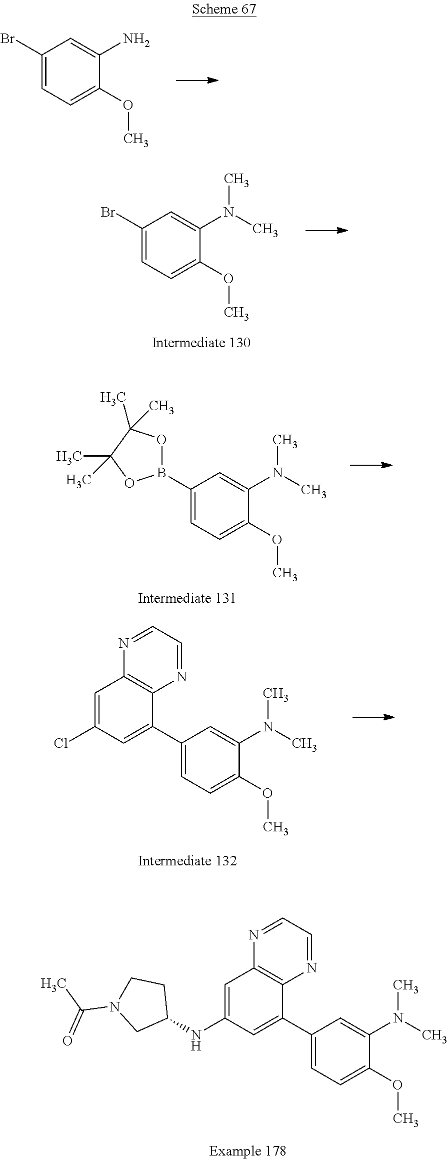

### Intermediate 130

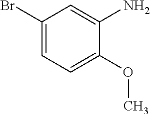

Roundbottom flask is charged with 5-bromo-2-methoxyaniline (300.00 mg; 1.48 mmol; 1.0 eq), methanol (6.0 mL), 37% formaldehyde solution in methanol (289.18 mg; 3.56 mmol; 2.40 eq.) and acetic acid (213.99 mg; 3.56 mmol; 2.40 eq.). RM is stirred at room temperature for 10 minutes. After this time RM is cooled down to 0° C. and sodiumcyanoborohydride (195.94 mg; 3.12 mmol; 2.10 eq.) is added. Reaction mixture is stirred in this temperature for 1h. Then the solvent is evaporated and reaction is quenched with saturated aqueous solution of NaHCO3 (5 mL) and extracted with DCM (3×5 mL). Combined organic extracts are washed with water (2×10 mL). Organic solvent is then evaporated to afford 5-bromo-2-methoxy-N,N-dimethylaniline (330.90 mg; yield 90.1%; 93.0% by UPLC) as a colorless oil.

### Intermediate 131

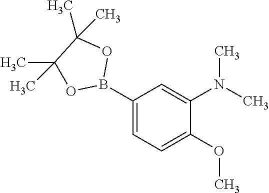

The product is prepared according to General Procedure 51, described for Intermediate 79 with 5-bromo-2-methoxy-N,N-dimethylaniline (Intermediate 130) (330.90 mg; 1.34 mmol; 1.0 eq.), 4,4,5,5-tetramethyl-2-(tetramethyl-1,3,2-dioxaborolan-2-yl)-1,3,2-dioxaborolane (441.50 mg; 1.74 mmol; 1.3 eq.), KOAc (262.51 mg; 2.67 mmol; 2.0 eq.) and dioxane (3.31 mL) and Pd(dppf)Cl2 (97.86 mg; 0.13 mmol; 0.10 eq.). Purification by FCC (hexane/EtOAc; gradient). 2-methoxy-N,N-dimethyl-5-(tetramethyl-1,3,2-dioxaborolan-2-yl)aniline (269.70 mg; yield 48.7%; 66.9% by HPLC) is obtained as an oil.

### Intermediate 132

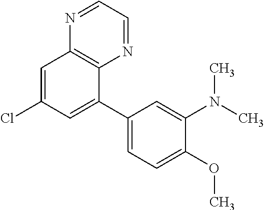 [SMILES: COc1ccc(-c2cc(Cl)cc3nccnc23)cc1N(C)C]

The product is prepared according to General Procedure 1, described for Intermediate 4 with 5-bromo-7-chloroquinoxaline (Intermediate 2) (158.51 mg; 0.65 mmol; 1.00 eq.), 2-methoxy-N,N-dimethyl-5-(tetramethyl-1,3,2-dioxaborolan-2-yl)aniline (Intermediate 130) (269.70 mg; 0.65 mmol; 1.00 eq.), DIPEA (0.23 mL; 1.30 mmol; 2.00 eq.), Pd(dppf)Cl₂ (47.61 mg; 0.07 mmol; 0.10 eq.), 1,4-dioxane (2.50 mL) and water (2.50 mL). Purification by FCC (hexane/EtOAc; gradient). 5-(7-chloroquinoxalin-5-yl)-2-methoxy-N,N-dimethylaniline (134.80 mg; yield 59.7%; 90.4% by HPLC) is obtained as a white yellow solid.

### Example 178

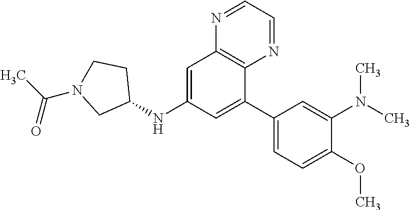 [SMILES: COc1ccc(-c2cc(N[C@H]3CCN(C(C)=O)C3)cc3nccnc23)cc1N(C)C]

The product is prepared according to General Procedure 2, described in Example 1 with 5-(7-chloroquinoxalin-5-yl)-2-methoxy-N,N-dimethylaniline (Intermediate 132) (50.00 mg; 0.16 mmol; 1.00 eq.), 1-[(3S)-3-aminopyrrolidin-1-yl]ethan-1-one hydrochloride (61.27 mg; 0.48 mmol; 3.00 eq.), NaOtBu (61.25 mg; 0.64 mmol; 4.00 eq.), BINAP (19.84 mg; 0.03 mmol; 0.20 eq.), Pd₂(dba)₃ (14.59 mg; 0.01 mmol; 0.10 eq.) and toluene (2.00 mL). Reaction is carried out in sealed tube at 1200° C. for 24 h. Purification by FCC (DCM/MeOH; gradient). Repurification by preparative HPLC is done 1-[(3S)-3-({8-[3-(dimethylamino)-4-methoxyphenyl]quinoxalin-6-yl}amino)pyrrolidin-1-yl]ethan-1-one (12.90 mg; yield 19.1%; 95.8% by HPLC) is obtained as a yellow powder.

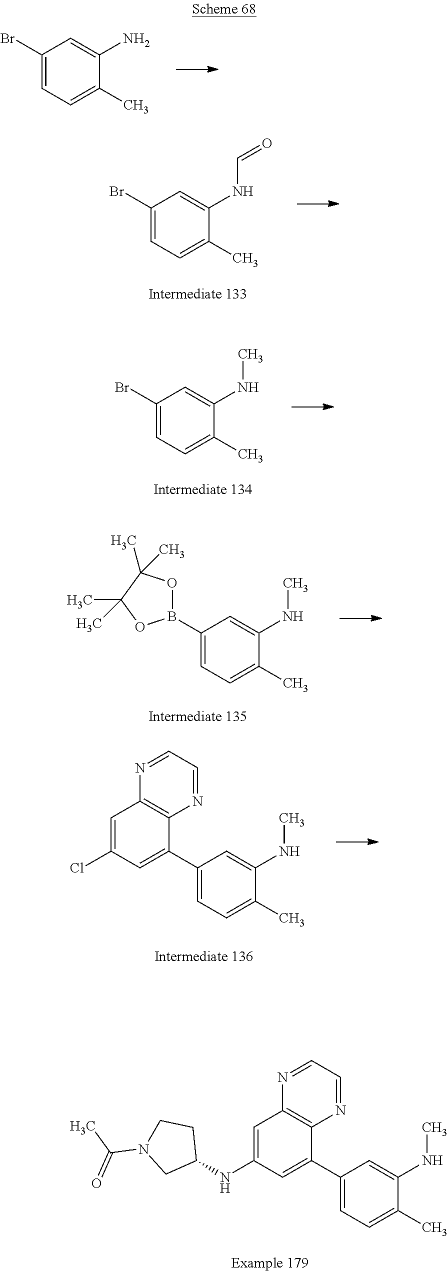

### Intermediate 133

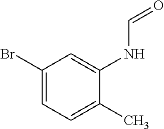

Roundbottom flask is charged with 5-bromo-2-methylaniline (1000 mg; 5.37 mmol; 1.0 eq.) and formic acid (1.22 mL; 32.25 mmol; 6.0 eq.). RM is cooled down to 0° C. and sodium formate (73.11 mg; 1.07 mmol; 0.2 eq.) is added in this temperature. RM is stirred at room temperature for 2 h. After this time RM is diluted with DCM and sodium formate is filtered off. Filtrate is washed with water and saturated aqueous solution of NaHCO₃. Organic solvent is dried over anhydrous Na₂SO₄ overnight. After this organic solvent is evaporated to afford N-(5-bromo-2-methylphenyl)formamide (869.50 mg; yield 68.8%; 91.0% by UPLC) as a brown solid.

### Intermediate 134

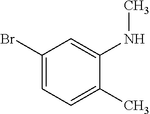

Roundbottom flask is charged with N-(5-bromo-2-methylphenyl)formamide (Intermediate 133) (869.50 mg; 2.82 mmol; 1.0 eq.) and anhydrous tetrahydrofuran (30.43 mL). RM is cooled down to 0° C. and lithium aluminium hydride 2.0 M solution in tetrahydrofuran (3.11 mL; 6.21 mmol; 2.2 eq.) is added in this temperature while RM is stirred. RM is allowed to warmed up to room temperature. RM is heated and stirred at reflux for 24 h. After this time RM is cooled to room temperature and water is added to RM. Then consecutively 5M aqueous solution of NaOH (10 mL) and water (30 mL). RM is stirred for 30 minutes and then extracted with EtOAc (3×30 mL). Organic solvents are then collected, combined and washed with brine (2 times). Then organic solvents are dried over Na₂SO₄ overnight. After this organic solvent is evaporated to afford 5-bromo-N,2-dimethylaniline (692.40 mg; yield 116.0%; 94.6% by UPLC) as a black oil.

### Intermediate 135

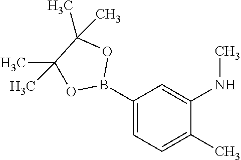

The product is prepared according to General Procedure 51, described for Intermediate 79 with with 5-bromo-N,2-dimethylaniline (Intermediate 134)(226.00 mg; 0.65 mmol; 1.0 eq.), 4,4,5,5-tetramethyl-2-(tetramethyl-1,3,2-dioxaborolan-2-yl)-1,3,2-dioxaborolane (215.91 mg; 0.85 mmol; 1.3 eq.), KOAc (128.37 mg; 1.31 mmol; 2.0 eq.) and dioxane (5.86 mL) and Pd(dppf)Cl2 (47.85 mg; 0.07 mmol; 0.10 eq.). Purification by FCC (hexane/EtOAc; gradient). N,2-dimethyl-5-(tetramethyl-1,3,2-dioxaborolan-2-yl)aniline (54.10 mg; yield 24.2%; 72.2% by HPLC) is obtained as an oil.

### Intermediate 136

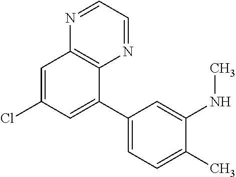 [SMILES: CNc1cc(-c2cc(Cl)cc3nccnc23)ccc1C]

The product is prepared according to General Procedure 1, described for Intermediate 4 with 5-bromo-7-chloroquinoxaline (Intermediate 2) (53.20 mg; 0.22 mmol; 1.00 eq.), N,2-dimethyl-5-(tetramethyl-1,3,2-dioxaborolan-2-yl)aniline (Intermediate 135) (54.00 mg; 0.22 mmol; 1.00 eq.), DIPEA (0.08 mL; 0.44 mmol; 2.00 eq.), Pd(dppf)Cl₂ (15.98 mg; 0.02 mmol; 0.10 eq.), 1,4-dioxane (2.50 mL) and water (2.50 mL). Purification by FCC (hexane/EtOAc; gradient). 5-(7-chloroquinoxalin-5-yl)-N,2-dimethylaniline (25.00 mg; yield 40.3%; 100% by HPLC) is obtained as a yellow crystals.

### Example 179

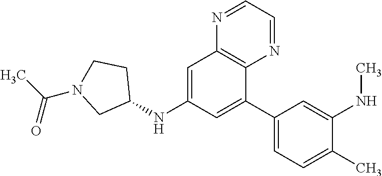 [SMILES: CNc1cc(-c2cc(N[C@H]3CCN(C(C)=O)C3)cc3nccnc23)ccc1C]

The product is prepared according to General Procedure 2, described in Example 1 with 5-(7-chloroquinoxalin-5-yl)-N,2-dimethylaniline (Intermediate 136) (23.90 mg; 0.08 mmol; 1.00 eq.), 1-[(3S)-3-aminopyrrolidin-1-yl]ethan-1-one hydrochloride (32.39 mg; 0.25 mmol; 3.00 eq.), NaOtBu (32.38 mg; 0.34 mmol; 4.00 eq.), BINAP (10.49 mg; 0.02 mmol; 0.20 eq.), Pd₂(dba)₃ (7.71 mg; 0.01 mmol; 0.10 eq.) and toluene (1.00 mL). Reaction is carried out in sealed tube at 120° C. for 24 h. Purification by FCC (DCM/MeOH; gradient). 1-[(3S)-3-({8-[4-methyl-3-(methylamino)phenyl]quinoxalin-6-yl}amino)pyrrolidin-1-yl]ethan-1-one (14.90 mg; yield 46.5%; 98.7% by HPLC) is obtained as a beige powder.

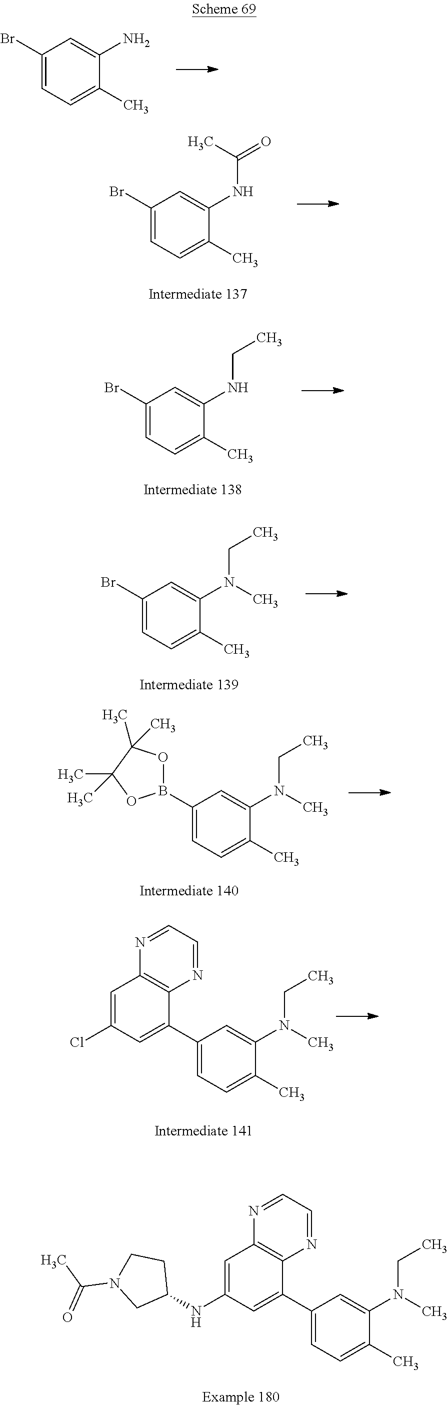

### Intermediate 137

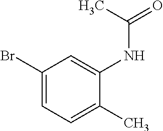

Roundbottom flask is charged with 5-bromo-2-methylaniline (400.00 mg; 2.15 mmol; 1.0 eq.), triethylamine (330.76 mL; 2.36 mmol; 1.1 eq.) and anhydrous dichloromethane (12 mL). Acetyl chloride (185.64 mg; 2.36 mmol; 1.1 eq.) is then added dropwise and reaction mixture is stirred at room temperature for 24h. After this time diethyl ether (24 mL) is added and mixture is washed with saturated aqueous solution of ammonium chloride (2×20 mL) and brine (2×20 mL). Organic layer is then dried over anhydrous Na₂SO₄ overnight. The second organic solvent is evaporated to afford N-(5-bromo-2-methylphenyl)acetamide (513.20 mg; yield 93.5%; 89.3% by UPLC) as brown solid.

### Intermediate 138

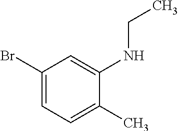

Roundbottom flask is charged with N-(5-bromo-2-methylphenyl)acetamide (Intermediate 137) (513.20 mg; 2.01 mmol; 1.0 eq.) and anhydrous tetrahydrofuran (17.96 mL). RM is cooled down to 0° C. and lithium aluminium hydride 2.0 M solution in tetrahydrofuran (2.21 mL; 4.42 mmol; 2.2 eq.) is added in this temperature while RM is stirred. RM is allowed to warmed up to room temperature. RM is heated and stirred at reflux for 24 h. After this time RM is cooled to room temperature and water is added to RM. Then consecutively 5M aqueous solution of NaOH (5 mL) and water (10 mL). RM is stirred for 30 minutes and then extracted with EtOAc (3×10 mL). Organic solvents are then collected, combined and washed with brine (2 times). Then organic solvents are dried over Na₂SO₄ overnight. After this organic solvent is evaporated to afford 5-bromo-N-ethyl-2-methylaniline (313.90 mg; yield 69.3%; 95.0% by UPLC) as a beige semisolid.

### Intermediate 139

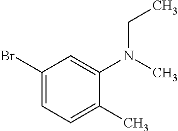

Roundbottom flask is charged with 5-bromo-N-ethyl-2-methylaniline (Intermediate 138) (313.90 mg; 1.39 mmol; 1.0 eq), methanol (7.50 mL), 37% formaldehyde solution in methanol (135.63 mg; 1.67 mmol; 1.20 eq.) and acetic acid (84.49 mg; 1.39 mmol; 1.0 eq.). RM is stirred at room temperature for 10 minutes. After this time RM is cooled down to 0° C. and sodiumcyanoborohydride (91.90 mg; 1.46 mmol; 1.05 eq.) is added. Reaction mixture is stirred in this temperature for 1 h. Then the solvent is evaporated and reaction is quenched with saturated aqueous solution of NaHCO₃ (5 mL) and extracted with EtOAc (3×5 mL). Combined organic extracts are washed with water (2×10 mL). Organic solvent is then evaporated and crude product is further purified by FCC (hexane/EtOAc, gradient) to afford 5-bromo-N-ethyl-N,2-dimethylaniline (150.40 mg; yield 47.3%; 100% by UPLC) as a beige oil.

### Intermediate 140

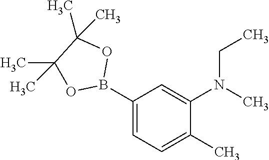

The product is prepared according to General Procedure 51, described for Intermediate 79 with 5-bromo-N-ethyl-N,2-dimethylaniline (Intermediate 139) (150.40 mg; 0.66 mmol; 1.0 eq.), 4,4,5,5-tetramethyl-2-(tetramethyl-1,3,2-dioxaborolan-2-yl)-1,3,2-dioxaborolane (217.64 mg; 0.86 mmol; 1.3 eq.), KOAc (129.41 mg; 1.32 mmol; 2.0 eq.) and dioxane (3.01 mL) and Pd(dppf)Cl2 (48.24 mg; 0.07 mmol; 0.10 eq.). Purification by FCC (hexane/EtOAc; gradient). N-ethyl-N,2-dimethyl-5-(tetramethyl-1,3,2-dioxaborolan-2-yl)aniline (119.00 mg; yield 29.0%; 47.9% by HPLC) is obtained as an oil.

### Intermediate 141

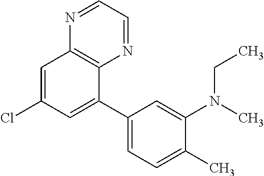 [SMILES: CCN(C)c1cc(-c2cc(Cl)cc3nccnc23)ccc1C]

The product is prepared according to General Procedure 1, described for Intermediate 4 with 5-bromo-7-chloroquinoxaline (Intermediate 2) (51.91 mg; 0.21 mmol; 1.00 eq.), N-ethyl-N,2-dimethyl-5-(tetramethyl-1,3,2-dioxaborolan-2-yl)aniline (Intermediate 140) (119.00 mg; 0.21 mmol; 1.00 eq.), DIPEA (0.07 mL; 0.43 mmol; 2.00 eq.), Pd(dppf)Cl₂ (15.59 mg; 0.02 mmol; 0.10 eq.), 1,4-dioxane (1.19 mL) and water (1.19 mL). Purification by FCC (hexane/EtOAc; gradient). 5-(7-chloroquinoxalin-5-yl)-N-ethyl-N,2-dimethylaniline (44.70 mg; yield 60.7%; 90.3% by HPLC) is obtained as a yellow solid.

### Example 180

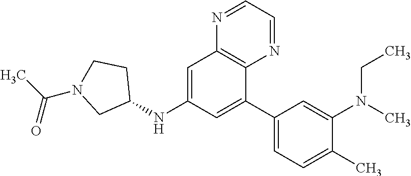 [SMILES: CCN(C)c1cc(-c2cc(N[C@H]3CCN(C(C)=O)C3)cc3nccnc23)ccc1C]

The product is prepared according to General Procedure 2, described in Example 1 with 5-(7-chloroquinoxalin-5-yl)-N-ethyl-N,2-dimethylaniline (Intermediate 141) (44.70 mg; 0.13 mmol; 1.00 eq.), 1-[(3S)-3-aminopyrrolidin-1-yl]ethan-1-one (58.26 mg; 0.39 mmol; 3.00 eq.), NaOtBu (49.76 mg; 0.52 mmol; 4.00 eq.), BINAP (16.12 mg; 0.03 mmol; 0.20 eq.), Pd₂(dba)₃ (11.85 mg; 0.01 mmol; 0.10 eq.) and toluene (1.12 mL). Reaction is carried out in sealed tube at 120° C. for 24 h. Purification by FCC (DCM/MeOH; gradient). Repurification by preparative HPLC is done. 1-[(3S)-3-[(8-{3-[ethyl(methyl)amino]-4-methylphenyl}quinoxalin-6-yl)amino]pyrrolidin-1-yl]ethan-1-one (6.00 mg; yield 10.8%; 94.2% by HPLC) is obtained as a yellow powder.

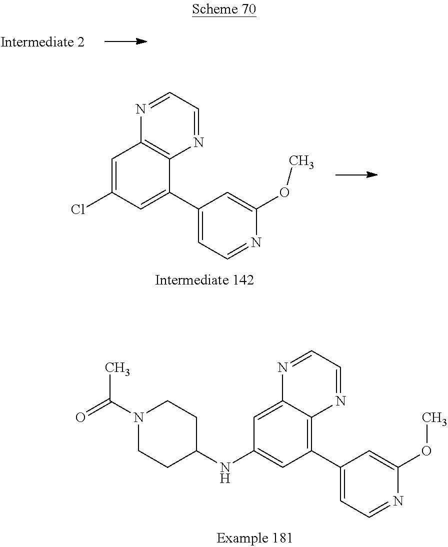 [SMILES: COc1cc(-c2cc(Cl)cc3nccnc23)ccn1 ; COc1cc(-c2cc(NC3CCN(C(C)=O)CC3)cc3nccnc23)ccn1]

### Intermediate 142

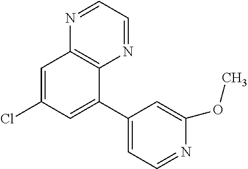 [SMILES: COc1cc(-c2cc(Cl)cc3nccnc23)ccn1]

The product is prepared according to General Procedure 1, described for Intermediate 4 with 5-bromo-7-chloroquinoxaline (Intermediate 2) (200.00 mg; 0.81 mmol; 1.00 eq.), (2-methoxypyridin-4-yl)boronic acid (137.21 mg; 0.89 mmol; 1.10 eq.), DIPEA (0.422 mL; 2.42 mmol; 3.00 eq.), Pd(dppf)Cl₂ (59.06 mg; 0.08 mmol; 0.10 eq.), 1,4-dioxane (1.70 mL) and water (0.60 mL). Purification by FCC (hexane/EtOAc; gradient). 7-chloro-5-(2-methoxypyridin-4-yl)quinoxaline (35.40 mg; yield 16.10%; 100% by HPLC) is obtained as a white fine powder.

### Example 181

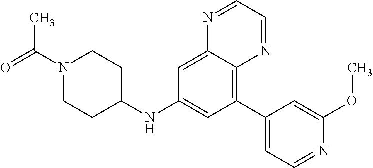 [SMILES: COc1cc(-c2cc(NC3CCN(C(C)=O)CC3)cc3nccnc23)ccn1]

The product is prepared according to General Procedure 2, described in Example 1 with 7-chloro-5-(2-methoxypyridin-4-yl)quinoxaline (Intermediate 142) (34.00 mg; 0.12 mmol; 1.00 eq.), 1-(4-aminopiperidin-1-yl)ethan-1-one (34.88 mg; 0.25 mmol; 2.00 eq.), NaOtBu (47.14 mg; 0.49 mmol; 4.00 eq.), BINAP (15.27 mg; 0.02 mmol; 0.20 eq.), Pd₂(dba)₃ (11.23 mg; 0.01 mmol; 0.10 eq.) and toluene (1.02 mL). Reaction is carried out in sealed tube at 120° C. for 24 h Purification by FCC (dichloromethane/methanol; gradient). 1-(4-{[8-(2-methoxypyridin-4-yl)quinoxalin-6-yl]amino}piperidin-1-yl)ethan-1-one (12.80 mg; yield 26.7%; 96.5% by HPLC) is obtained as a brown yellow powder.

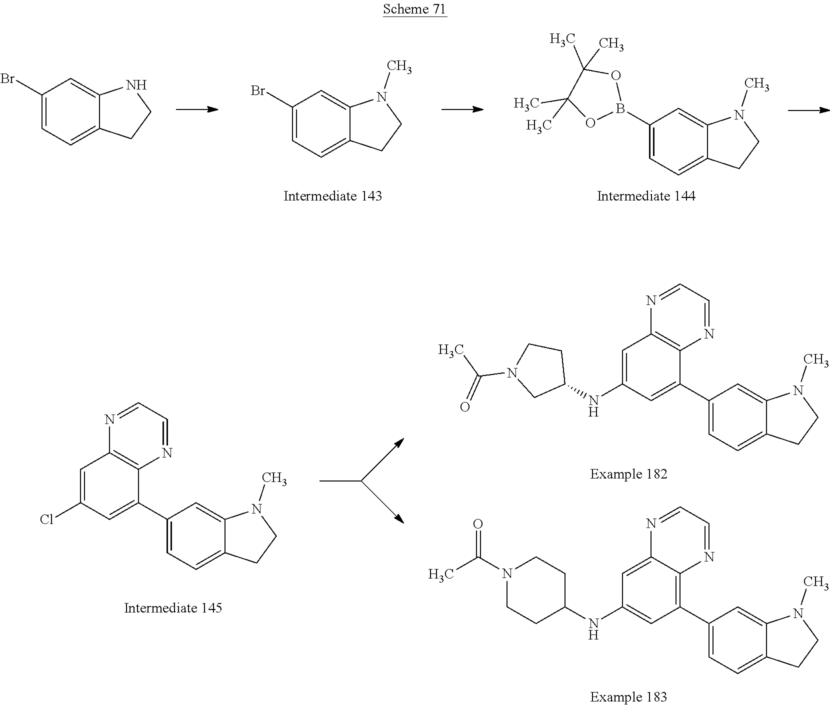 [SMILES: Brc1ccc2c(c1)NCC2 ; CN1CCc2ccc(Br)cc21 ; CN1CCc2ccc(B3OC(C)(C)C(C)(C)O3)cc21 ; CN1CCc2ccc(-c3cc(Cl)cc4nccnc34)cc21 ; CC(=O)N1CCC(Nc2cc(-c3ccc4c(c3)N(C)CC4)c3nccnc3c2)CC1 ; CC(=O)N1CC[C@H](Nc2cc(-c3ccc4c(c3)N(C)CC4)c3nccnc3c2)C1]

### Intermediate 143—General Procedure 55

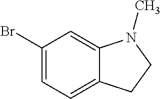

Roundbottom flask is charged with 6-bromo-2,3-dihydro-1H-indole (500.00 mg; 2.52 mmol; 1.00 eq.) methanol (7.50 ml), formaldehyde (245.84 mg; 3.03 mmol; 1.20 eq.) and acetic acid (153.13 mg; 2.52 mmol; 1.00 eq.). RM is stirred at rt for 10 minutes, then coolled down to 0° C. and sodiumcyanoborohydride (166.57 mg; 2.65 mmol; 1.05 eq.) is added. Reaction is stirred at this temperature for 1h. Then the solvent is removed under reduced pressure and reaction is quenched with saturated aqueous NaHCO₃. Product is taken up to the DCM by extraction (5×3 mL). The combined organic layers are washed two times with water and evaporated to give 6-bromo-1-methyl-2,3-dihydro-1H-indole (516.60 mg; 2.41 mmol; 95.5%; 99.% by UPLC) as na oil.

### Intermediate 144

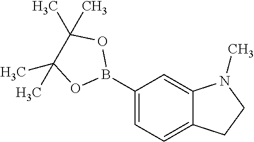 [SMILES: CN1CCc2ccc(B3OC(C)(C)C(C)(C)O3)cc21]

The product is prepared according to General Procedure 51, described for Intermediate 79 with 66-bromo-1-methyl-2,3-dihydro-1H-indole (Intermediate 143) (619.90 mg; 2.89 mmol; 1.0 eq.), 4,4,5,5-tetramethyl-2-(tetramethyl-1,3,2-dioxaborolan-2-yl)-1,3,2-dioxaborolane (955.25 mg; 3.76 mmol; 1.3 eq.), KOAc (567.98 mg; 5.79 mmol; 2.0 eq.) and dioxane (6.20 mL) and Pd(dppf)Cl2 (211.73 mg; 0.29 mmol; 0.10 eq.). In this case extraction was done instead of FCC: Crude product after evaporation is diluted with EtOAc and washed with water and brine (2 times each). 1-methyl-6-(tetramethyl-1,3,2-dioxaborolan-2-yl)-1H-indazole (990.60 mg; yield 132.10%; 90.5% by HPLC) is obtained as a dark oil contaminated with unreacted substrate.

### Intermediate 145

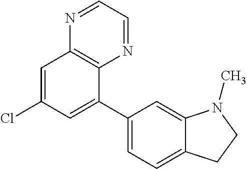 [SMILES: CN1CCc2ccc(-c3cc(Cl)cc4nccnc34)cc21]

The product is prepared according to General Procedure 1, described for Intermediate 4 with 5-bromo-7-chloroquinoxaline (Intermediate 2) (281.87 mg; 1.16 mmol; 1.00 eq.), 1-methyl-6-(tetramethyl-1,3,2-dioxaborolan-2-yl)-2,3-dihydro-1H-indole (Intermediate 144) (300 mg; 1.16 mmol; 1.00 eq.), DIPEA (0.40 mL; 2.32 mmol; 2.00 eq.), Pd(dppf)Cl₂ (84.67 mg; 0.12 mmol; 0.10 eq.), 1,4-dioxane (2.50 mL) and water (2.50 mL). Purification by FCC (hexane/EtOAc; gradient). 7-chloro-5-(1-methyl-2,3-dihydro-1H-indol-6-yl)quinoxaline (Intermediate 145) (223.20 mg; yield 57.4%; 88.0% by UPLC) is obtained as a yellow crystals.

### Example 182

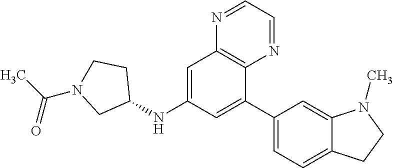 [SMILES: CC(=O)N1CC[C@H](Nc2cc(-c3ccc4c(c3)N(C)CC4)c3nccnc3c2)C1]

The product is prepared according to General Procedure 2, described in Example 1 with 7-chloro-5-(1-methyl-2,3-dihydro-1H-indol-6-yl)quinoxaline (Intermediate 145) (50.00 mg; 0.15 mmol; 1.00 eq.), 1-[(3S)-3-amino-pyrrolidin-1-yl]ethan-1-one hydrochloride (73.48 mg; 0.45 mmol; 3.00 eq.), NaOtBu (57.19 mg; 0.60 mmol; 4.00 eq.), BINAP (18.53 mg; 0.03 mmol; 0.20 eq.), Pd₂(dba)₃ (13.62 mg; 0.01 mmol; 0.10 eq.) and toluene (1.50 mL). Reaction is carried out in sealed tube at 120° C. for 24 h. Purification by FCC (DCM/MeOH; gradient). 1-[(3S)-3-{[8-(1-methyl-2,3-dihydro-1H-indol-6-yl)quinoxalin-6-yl]amino}pyrrolidin-1-yl]ethan-1-one (8.90 mg; yield 14.7%; 95.4% by HPLC) is obtained as a green powder.

### Example 183

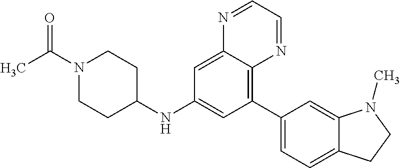 [SMILES: CC(=O)N1CCC(Nc2cc(-c3ccc4c(c3)N(C)CC4)c3nccnc3c2)CC1]

The product is prepared according to General Procedure 2, described in Example 1 with 7-chloro-5-(1-methyl-2,3-dihydro-1H-indol-6-yl)quinoxaline (Intermediate 145) (50.00 mg; 0.15 mmol; 1.00 eq.), 1-(4-aminopiperidin-1-yl)ethan-1-one (42.31 mg; 0.30 mmol; 2.00 eq.), NaOtBu (57.19 mg; 0.60 mmol; 4.00 eq.), BINAP (18.53 mg; 0.03 mmol; 0.20 eq.), Pd₂(dba)₃ (13.62 mg; 0.01 mmol; 0.10 eq.) and toluene (1.50 mL). Reaction is carried out in sealed tube at 120° C. for 24 h. Purification by FCC (DCM/MeOH; gradient). 1-(4-{[8-(1-methyl-2,3-dihydro-1H-indol-6-yl)quinoxalin-6-yl]amino}piperidin-1-yl)ethan-1-one (22.40 mg; yield 35.8%; 95.5% by HPLC) is obtained as a green powder.

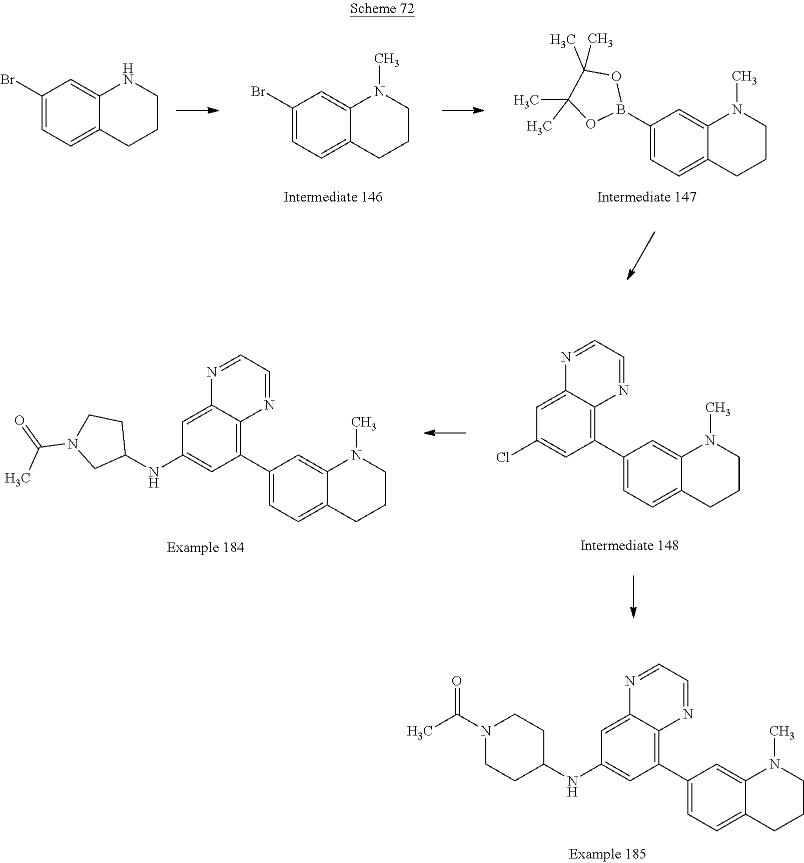 [SMILES: Brc1ccc2c(c1)NCCC2 ; CN1CCCc2ccc(Br)cc21 ; CN1CCCc2ccc(B3OC(C)(C)C(C)(C)O3)cc21 ; CC(=O)N1CCC(Nc2cc(-c3ccc4c(c3)N(C)CCC4)c3nccnc3c2)C1 ; CN1CCCc2ccc(-c3cc(Cl)cc4nccnc34)cc21 ; CC(=O)N1CCC(Nc2cc(-c3ccc4c(c3)N(C)CCC4)c3nccnc3c2)CC1]

### Intermediate 146

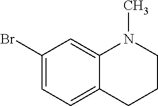

The product is prepared according to General Procedure 55 for N-methylation, described for Intermediate 143 with 7-bromo-1,2,3,4-tetrahydroquinoline (300.00 mg; 1.41 mmol; 1.00 eq.), MeOH (6.000 ml), formaldehyde 37% (0.126 ml; 1.70 mmol; 1.20 eq.), acetic acid (0.082 ml; 1.43 mmol; 1.01 eq.) and sodium cyanoborohydride (93.00 mg; 1.48 mmol; 1.05 eq.). Time of stirring of RM in an ice bath is extended to 2 h. 7-bromo-1-methyl-1,2,3,4-tetrahydroquinoline (234.00 mg; yield 72.4%; 99.00% by UPLC) is obtained as a light yellow oil.

### Intermediate 147

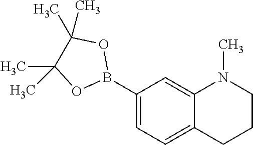 [SMILES: CN1CCCc2ccc(B3OC(C)(C)C(C)(C)O3)cc21]

The product is prepared according to General Procedure 1, described for Intermediate 4 with 7-bromo-1-methyl-1,2,3,4-tetrahydroquinoline (Intermediate 146) (230.00 mg; 1.01 mmol; 1.00 eq.), bis(pinacolato)diboron (332.44 mg; 1.31 mmol; 1.30 eq.), Pd(dppf)Cl2, (7.37 mg; 0.01 mmol; 0.01 eq.), KOAc (197.66 mg; 2.01 mmol; 2.00 eq.) and 1,4-dioxane (5.000 ml). Reaction is carried out overnight at 100° C. After filtration through Celite® diluted RM is partitioned between EtOAc and water. The organic phase is dried and evaporated. Purification by FCC (hexane/EtOAc; gradient) to give 1-methyl-7-(tetramethyl-1,3,2-dioxaborolan-2-yl)-1,2,3,4-tetrahydroquinoline (233.00 mg; yield 82.7%; 97.6% by UPLC) as a yellow oil.

### Intermediate 148

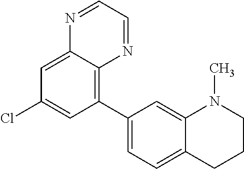 [SMILES: CN1CCCc2ccc(-c3cc(Cl)cc4nccnc34)cc21]

The product is prepared according to modified General Procedure 1, described for Intermediate 4 with 1-methyl-7-(tetramethyl-1,3,2-dioxaborolan-2-yl)-1,2,3,4-tetrahydroquinoline (Intermediate 147) (233.00 mg; 0.83 mmol; 1.00 eq.), 5-bromo-7-chloroquinoxaline (Intermediate 2) (203.71 mg; 0.83 mmol; 1.00 eq.), Pd(dppf)Cl₂ (60.9 mg; 0.08 mmol; 0.10 eq.), DIPEA (0.435 ml; 2.50 mmol; 3.00 eq.), 1,4-dioxane (7.000 ml) and water (3.000 ml). Reaction is carried out for 1 h at MW 120° C. Then it is diluted with EtOAc and filtered through Celite®. The filtrate is evaporated under reduced pressure and the residue is purified by FCC (hexane/EtOAc; gradient) to give 7-chloro-5-(1-methyl-1,2,3,4-tetrahydroquinolin-7-yl)quinoxaline (132.80 mg; yield 50.5%; 98.00% by UPLC) as a yellow glass.

### Example 184

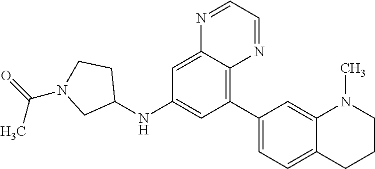 [SMILES: CC(=O)N1CCC(Nc2cc(-c3ccc4c(c3)N(C)CCC4)c3nccnc3c2)C1]

The product is prepared according to General Procedure 2, described in Example 1 with 7-chloro-5-(1-methyl-1,2,3,4-tetrahydroquinolin-7-yl)quinoxaline (Intermediate 148) (50.00 mg; 0.16 mmol; 1.00 eq.), 1-(3-aminopyrrolidin-1-yl)ethan-1-one (64.00 mg; 0.50 mmol; 3.16 eq.), NaOtBu (19.76 mg; 0.21 mmol; 1.30 eq.), BINAP (19.70 mg; 0.03 mmol; 0.20 eq.), Pd₂(dba)₃ (14.48 mg; 0.02 mmol; 0.10 eq.) and toluene (1.500 ml). Reaction is carried out overnight at 110° C. Then RM is diluted with EtOAc and filtered through Celite®. The filtrate is evaporated to dryness and the residue is purified by FCC (DCM/MeOH; gradient) to give 1-(3-{[8-(1-methyl-1,2,3,4-tetrahydroquinolin-7-yl)quinoxalin-6-yl]amino}pyrrolidin-1-yl)ethan-1-one (28.20 mg; yield 43.7%; 98.50% by HPLC) as an orange powder.

### Example 185

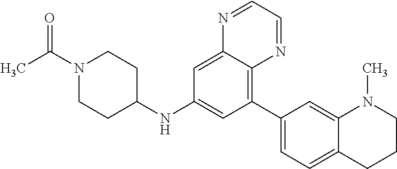 [SMILES: CC(=O)N1CCC(Nc2cc(-c3ccc4c(c3)N(C)CCC4)c3nccnc3c2)CC1]

The product is prepared according to General Procedure 2, described in Example 1 with 7-chloro-5-(1-methyl-1,2,3,4-tetrahydroquinolin-7-yl)-quinoxaline (Intermediate 148) (74.00 mg; 0.23 mmol; 1.00 eq.), 1-(4-aminopiperidin-1-yl)ethan-1-one (99.86 mg; 0.70 mmol; 3.00 eq.), NaOtBu (29.25 mg; 0.30 mmol; 1.30 eq.), BINAP (29.15 mg; 0.05 mmol; 0.20 eq.) Pd₂(dba)₃ (21.44 mg; 0.02 mmol; 0.10 eq.) and toluene (2.000 ml). Reaction is carried out overnight at 120° C. Then RM is diluted with EtOAc and filtered through Celite®. The filtrate is evaporated to dryness and the residue is purified by FCC (DCM/MeOH; gradient) to give 1-(4-{[8-(1-methyl-1,2,3,4-tetrahydroquinolin-7-yl)quinoxalin-6-yl]amino}piperidin-1-yl)ethan-1-one (20.40 mg; yield 20.8%; 99.10% by HPLC) as a brown powder.

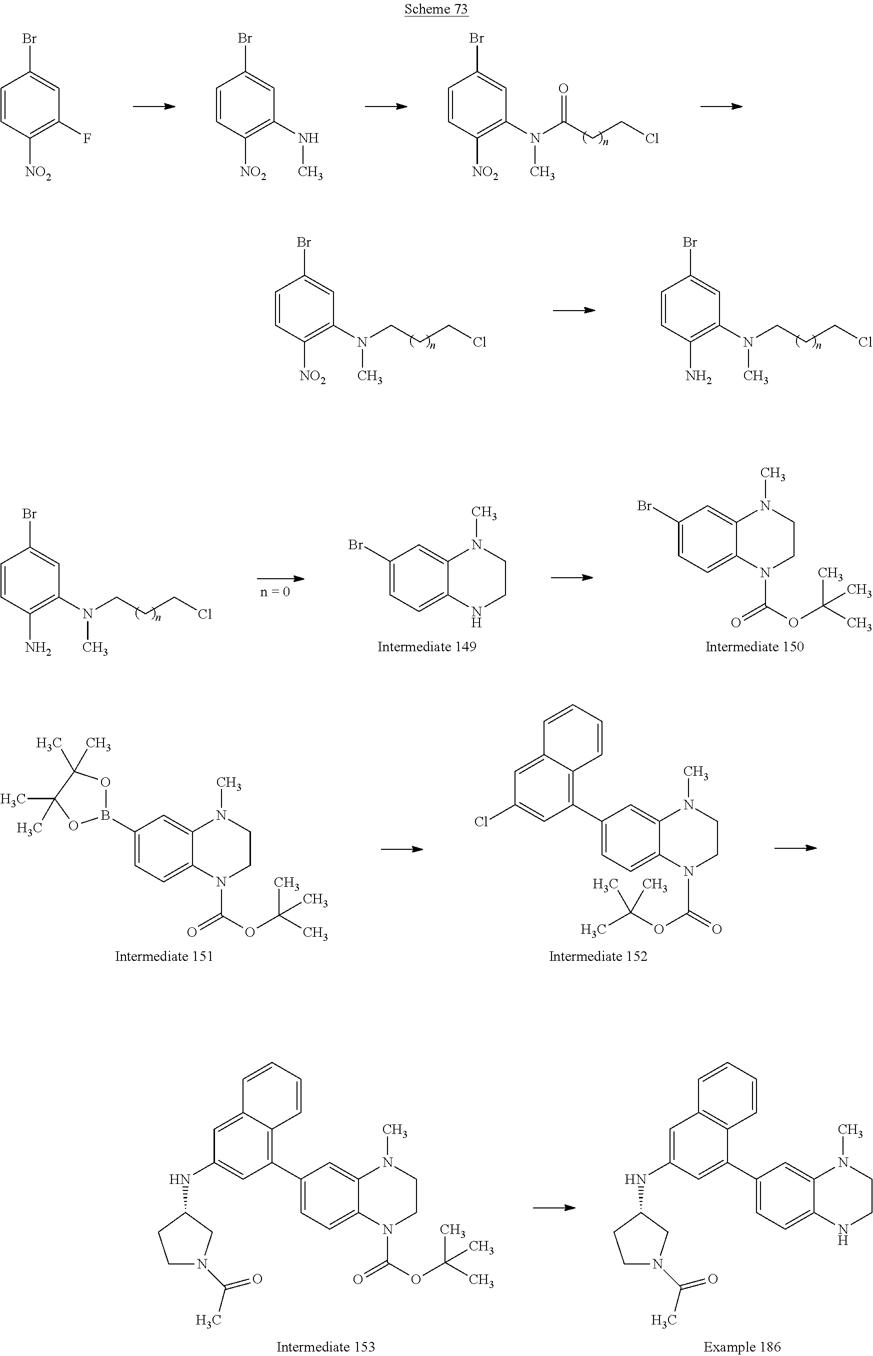

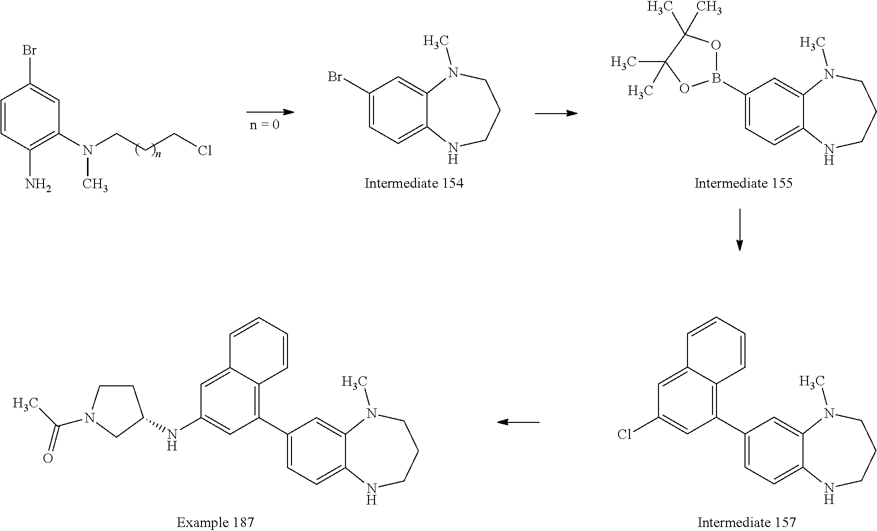

### Intermediate 149—General Procedure 56

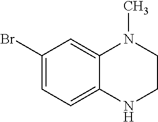

Product is obtained according to the procedures described in literature (Step 1 WO 2010027500, Steps 2, 3, 4 and 5 WO 2014008214).

Step 1. 2M Methylamine in ethanol (4.9 ml; 9.73 mmol; 2.00 eq.) is dropped in over 5 min at rt to the stirred solution of 4-bromo-2-fluoro-1-nitrobenzene (1.07 g; 4.86 mmol; 1.00 eq.) in ethanol (10.00 ml). RM is stirred at rt for 30 min, then solvent is evaporated and residue is triturated with water to remove methylamine hydrofluoride. The remaining residue is collected by filtration, washed with water and dried at 60 C in an oven to give 5-bromo-N-methyl-2-nitroaniline (1.07 g; 4.62 mmol; yield 95.0%; 100% by UPLC) as orange thin needles.

Step 2. To the solution of 5-bromo-N-methyl-2-nitroaniline (1.10 g; 4.76 mmol; 1.00 eq.) in anhydrous THF (11.00 ml) potassium carbonate (1.32 g; 9.52 mmol; 2.00 eq.) is added followed by addition of chloroacetyl chloride (0.57 ml; 7.14 mmol; 1.50 eq.). RM is refluxed for 3 h until SM decay on TLC. Then RM is diluted in 100 ml of EtOAc and extracted with water (3×10 ml). Organic layer is washed with brine and dried over Na₂SO₄. Drying agent is filtered off and solvent is evaporated under reduced pressure to give N-(5-bromo-2-nitrophenyl)-3-chloro-N-methylpropanamide (1.45 g; 4.67 mmol; 98.0%; 99% by UPLC) as an yellow oil.

Step 3. The mixture of N-(5-bromo-2-nitrophenyl)-3-chloro-N-methylpropanamide and THF borane complex (14.63 ml; 14.63 mmol; 4.50 eq.) is stirred at rt for 18 h under argon atmosphere. Then RM is quenched by addition of cold methanol. Resulted mixture is evaporated to give 5-bromo-N-(3-chloropropyl)-N-methyl-2-nitroaniline (0.88 g; 2.93 mmol; yield 90.1%; 97.4% by UPLC) as dark yellow oil.

Step 4. 5-Bromo-N-(3-chloropropyl)-N-methyl-2-nitroaniline (0.88 g; 3.01 mmol; 1.00 eq.), acetic acid (15.00 ml) and iron (0.84 g; 15.04 mmol; 5.00 eq.) are heated and sonificated at 50° C. for 1 h and then additionally stirred for 2 h at rt. Unreacted iron is collected by magnet and resulted slurry is diluted with water (100 mL), basified with 2M NaOH to pH 8. Resulted mixture is filtered through Celite® pad. Filtrate is extracted with Et₂O (3×50 mL). Combined organic layers are washed with water, brine and dried over MgSO₄. Drying agent is filtrated off and filtrate is evaporated in vacuo. 5-Bromo-1-N-(3-chloropropyl)-1-N-methylbenzene-1,2-diamine (0.68 g; 2.34 mmol; yield 77.7%; 91% by UPLC) is obtained as an yellow oil.

Step 5. Sealed tube is charged with 5-bromo-1-N-(3-chloropropyl)-1-N-methylbenzene-1,2-diamine (0.68 g; 2.34 mmol; 1.00 eq.), potassium iodide (1.16 g; 7.01 mmol; 3.00 eq), potassium carbonate (0.65 g; 4.67 mmol; 2.00 eq) and DMF (10 mL). Resulted mixture is stirred on an oil bath preheated to 80 C for 3 h. Then DMF is evaporated in vacuo and residue is portioned by Et₂O 50 mL and water (50 mL). Organic layer is collected and water layer is additionally extracted with Et₂O (2×50 mL). Combined organic layers are washed with water and brine, dried over MGSO₄ and evaporated. Purification by FCC (hexane/EtOAc, gradient). 7-Bromo-1-methyl-1,2,3,4-tetrahydroquinoxaline (Intermediate 147) (0.311 g, 1.22 mmol; yield 52%; 87% by UPLC) is obtained as yellow crystallizing oil.

### Intermediate 150

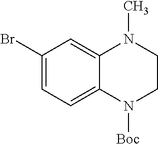 [SMILES: CN1CCN(C(=O)OC(C)(C)C)c2ccc(Br)cc21]

The Round bottom flask is charged with 7-Bromo-1-methyl-1,2,3,4-tetrahydroquinoxaline (Intermediate 149) (0.31 g, 1.22 mmol; 1.00 eq), Boc₂O (0.533 g; 4.44 mmol; 2 eq), DIPEA (0.43 mL; 2.44 mmol; 2 eq), DMAP (15 mg; 0.12 mmol; 0.1 eq) and anhydrous DCM (10 mL). RM is stirred at rt overnight. Then RM is diluted with DCM and extracted successively with water, and brine, dried over Na₂SO₄. Drying agent is filtered off and solvent evaporated. Crude product is purified by FCC (hexane/AcORe, gradient). tert-Butyl 6-bromo-4-methyl-1,2,3,4-tetrahydroquinoxaline-1-carboxylate (155 mg; 0.46 mmol; yield 37%; 97% by UPLC) is obtained as an oil.

### Intermediate 151

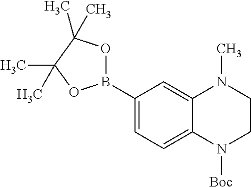 [SMILES: CN1CCN(C(=O)OC(C)(C)C)c2ccc(B3OC(C)(C)C(C)(C)O3)cc21]

The product is prepared according to General Procedure 51 for Miyaura coupling borylation, described for Intermediate 79 with tert-Butyl 6-bromo-4-methyl-1,2,3,4-tetrahydroquinoxaline-1-carboxylate (Intermediate 150) (155 mg; 0.46 mmol; 1 eq.), 4,4,5,5-tetramethyl-2-(tetramethyl-1,3,2-dioxaborolan-2-yl)-1,3,2-dioxaborolane (151.7 mg; 0.60 mmol; 1.30 eq.), KOAc (112.7 mg; 1.15 mmol; 2.50 eq.) and [1,4]-dioxane (5.00 ml). Resulted slurry is flashed with argon and Pd(dppf)Cl₂ (33.6 mg; 0.05 mmol; 0.10 eq.) is added under argon atmosphere. Reaction is carried out for 18 h at 100° C. Crude tert-butyl 4-methyl-6-(tetramethyl-1,3,2-dioxaborolan-2-yl)-1,2,3,4-tetrahydroquinoxaline-1-carboxylate ester (314.0 mg; 84% by UPLC) is obtained as dark oil which is used in next step without further purification.

### Intermediate 152

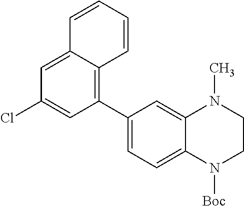 [SMILES: CN1CCN(C(=O)OC(C)(C)C)c2ccc(-c3cc(Cl)cc4ccccc34)cc21]

Product is prepared according to General Procedure 54, described for Intermediate 117 with 5-bromo-7-chloroquinoxaline (Intermediate 2) (100.00 mg; 0.40 mmol; 1.00 eq.), tert-butyl 4-methyl-6-(tetramethyl-1,3,2-dioxaborolan-2-yl)-1,2,3,4-tetrahydroquinoxaline-1-carboxylate ester (Intermediate 151) (310 mg; 0.44 mmol; 1.10 eq.), caesium carbonate (263. mg; 0.81 mmol; 2.00 eq.), Pd(dppf)Cl₂ (29.5 mg; 0.04 mmol; 0.10 eq.), water (3 mL) and [1,4]-dioxane. RM is heated to 120° C. and irradiated with MW in the Biotage Initiator unit for 30 min. Purification by FCC (hexane/EtOAc; gradient). tert-Butyl 6-(3-chloronaphthalen-1-yl)-4-methyl-1,2,3,4-tetrahydroquinoxaline-1-carboxylate (72.4 mg; 0.17 mmol; yield 43.2%; 99% by UPLC) is obtained as yellow film.

### Intermediate 153

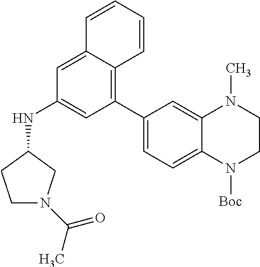 [SMILES: CC(=O)N1CC[C@H](Nc2cc(-c3ccc4c(c3)N(C)CCN4C(=O)OC(C)(C)C)c3ccccc3c2)C1]

The product is prepared according to the modified General Procedure 2, described in Example 1 with tert-Butyl 6-(3-chloronaphthalen-1-yl)-4-methyl-1,2,3,4-tetrahydroquinoxaline-1-carboxylate (Intermediate 152) (72.4 mg; 0.17 mmol; 1 eq), 1-[(3S)-3-aminopyrrolidin-1-yl]ethan-1-one (68 mg; 0.53 mmol; 3.00 eq.), NaOtBu (68 mg; 0.70 mmol; 4.00 eq.), BINAP (22 mg; 0.04 mmol; 0.20 eq.), Pd₂(dba)₃ (16 mg; 0.02 mmol; 0.10 eq.) and [1,4]-Dioxane (2.0 ml). Reaction is carried out in sealed tube at 120° C. for 18 h. Purification by FCC (MeOH/DCM, gradient). tert-Butyl 6-(3-{[(3S)-1-acetylpyrrolidin-3-yl]amino}naphthalen-1-yl)-4-methyl-1,2,3,4-tetrahydroquinoxaline-1-carboxylate (69.9 mg; 0.14 mmol; yield 78.9%; 100% by UPLC) is obtained as yellow foam.

### Example 186

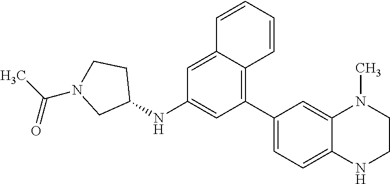 [SMILES: CC(=O)N1CC[C@H](Nc2cc(-c3ccc4c(c3)N(C)CCN4)c3ccccc3c2)C1]

The MW reacting vessel is charged with tert-Butyl 6-(3-{[(3S)-1-acetylpyrrolidin-3-yl]amino}naphthalen-1-yl)-4-methyl-1,2,3,4-tetrahydroquinoxaline-1-carboxylate (Intermediate 153) (69.9 mg; 0.14 mmol; 1.0 eq), PTSA (53 mg; 0.28 mmol; 2.00 eq.), anhydrous toluene (2.00 ml) and methanol (0.50 ml). The vessel is sealed and RM is heated to 100° C. and irradiated with MW in the Biotage Initiator unit for 10 min. Resulted deep red solution was portioned by DCM and water and basified to pH 8-10 with 2M NaOH (organic layer turns from red to neon green). Organic layer is successively washed with water and brine, dried over Na₂SO₄ and evaporated in vacuo. Resulted residue is purified by FCC (MeOH/EtOAc; gradient). 1-[(3S)-3-{[8-(5-Methyl-2,3,4,5-tetrahydro-1H-1,5-benzodiazepin-7-yl)quinoxalin-6-yl]amino}pyrrolidin-1-yl]ethan-1-one (36.5 mg; 0.09 mmol; yield 63.5%; 97.4% by HPLC) is obtained as orange-red foam.

### Intermediate 154

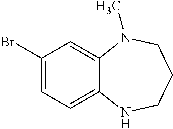

Product is prepared according to General Procedure 56 described for Intermediate 149. Starting from Step 2 with 5-bromo-N-methyl-2-nitroaniline (0.50 g; 2.16 mmol; 1.00 eq.) four subsequent steps is reproduced. The 8-bromo-1-methyl-2,3,4,5-tetrahydro-1H-1,5- (0.282 g; 1.12 mmol; yield 52% calculated after 4 steps; 96% by UPLC) is obtained as light yellow oil.

### Intermediate 155

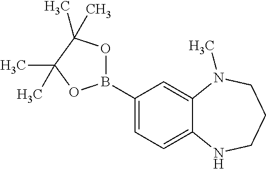 [SMILES: CN1CCCNc2ccc(B3OC(C)(C)C(C)(C)O3)cc21]

The product is prepared according to General Procedure 51, described for Intermediate 79 with 8-bromo-1-methyl-2,3,4,5-tetrahydro-1H-1,5-benzodiazepine (Intermediate 154) (0.282 g; 1.12 mmol; 1.0 eq.), 4,4,5,5-tetramethyl-2-(tetramethyl-1,3,2-dioxaborolan-2-yl)-1,3,2-dioxaborolane (0.386 g; 1.52 mmol; 1.3 eq.), KOAc (286.94 mg; 2.92 mmol; 2.50 eq.) and [1,4]-Dioxane (5.00 ml). Resulted slurry is flashed with argon and Pd(dppf)Cl₂ (85.57 mg; 0.12 mmol; 0.10 eq.) is added under argon atmosphere. Reaction is carried out for 18 h at 100° C. Crude 1-methyl-8-(tetramethyl-1,3,2-dioxaborolan-2-yl)-2,3,4,5-tetrahydro-1H-1,5-benzodiazepine (507 mg; 1.18 mmol; yield 100%; 67% by UPLC) is obtained as dark oil which is used in next step without further purification.

### Intermediate 156

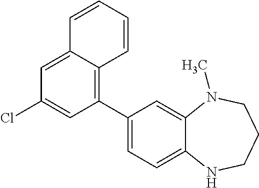 [SMILES: CN1CCCNc2ccc(-c3cc(Cl)cc4ccccc34)cc21]

Product is prepared according to General Procedure 54, described for Intermediate 117 with 5-bromo-7-chloroquinoxaline (Intermediate 2) (100.00 mg; 0.40 mmol; 1.00 eq.), 1-methyl-8-(tetramethyl-1,3,2-dioxaborolan-2-yl)-2,3,4,5-tetrahydro-1H-1,5-benzodiazepine (Intermediate 155) (507 mg; 1.18 mmol; yield 100%; 1.10 eq.), cessium carbonate (658. mg; 2.02 mmol; 2.00 eq.), Pd(dppf)Cl₂ (74 mg; 0.04 mmol; 0.10 eq.), water (3 mL) and [1,4]-dioxane 95 mL). RM is heated to 120° C. and irradiated with MW in the Biotage Initiator unit for 30 min. Purification by FCC (hexane/EtOAc; gradient). 8-(3-Chloronaphthalen-1-yl)-1-methyl-2,3,4,5-tetrahydro-1H-1,5-benzodiazepine (122.40 mg; 0.31 mmol; yield 30.6%; 82% by UPLC) is obtained as orange-red oil.

### Example 187

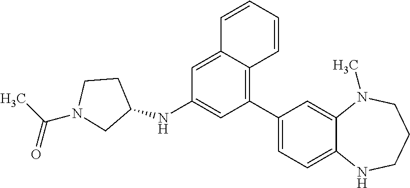 [SMILES: CC(=O)N1CC[C@H](Nc2cc(-c3ccc4c(c3)N(C)CCCN4)c3ccccc3c2)C1]

The product is prepared according to the modified General Procedure 2, described in Example 1 with 8-(3-Chloronaphthalen-1-yl)-1-methyl-2,3,4,5-tetrahydro-1H-1,5-benzodiazepine (Intermediate 156) (60 mg; 0.15 mmol; 1 eq), 1-[(3S)-3-aminopyrrolidin-1-yl]ethan-1-one (59 mg; 0.46 mmol; 3.00 eq.), NaOtBu (59 mg; 0.61 mmol; 4.00 eq.), BINAP (19 mg; 0.03 mmol; 0.20 eq.), Pd₂(dba)₃ (14 mg; 0.02 mmol; 0.10 eq.) and [1,4]-dioxane (2.0 ml). Reaction is carried out in sealed tube at 120° C. for 18 h. Purification by FCC (MeOH/DCM, gradient). 1-[(3S)-3-{[4-(5-methyl-2,3,4,5-tetrahydro-1H-1,5-benzodiazepin-7-yl)naphthalen-2-yl]amino}pyrrolidin-1-yl]ethan-1-one (33.0 mg; 0.08 mmol; yield 50.1%; 96.9% by HPLC) is obtained as brown foam.

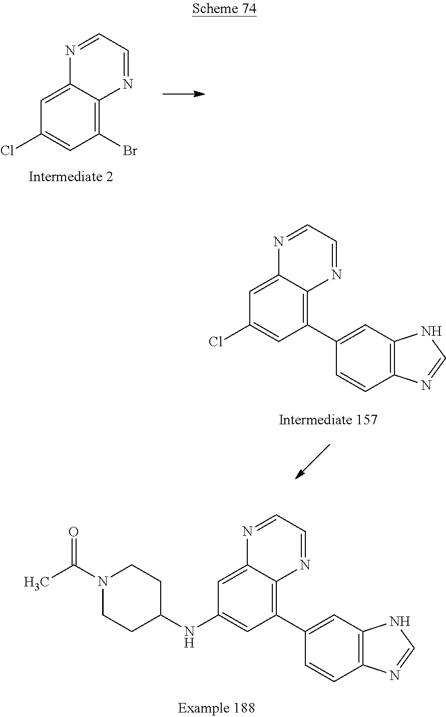 [SMILES: Clc1cc(Br)c2nccnc2c1 ; Clc1cc(-c2ccc3nc[nH]c3c2)c2nccnc2c1 ; CC(=O)N1CCC(Nc2cc(-c3ccc4nc[nH]c4c3)c3nccnc3c2)CC1]

### Intermediate 157

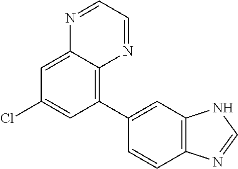 [SMILES: Clc1cc(-c2ccc3nc[nH]c3c2)c2nccnc2c1]

Product is prepared according to General Procedure 54, described for Intermediate 117 with 5-bromo-7-chloroquinoxaline (Intermediate 2) (0.23 g; 1.14 mmol; 1.00 eq.), 6-(tetramethyl-1,3,2-dioxaborolan-2-yl)-1H-1,3-benzo-diazole (279 mg; 2.83 mmol; 3.00 eq.), DIPEA (0.49 mL; 2.83 mmol; 3.00 eq.), Pd(dppf)Cl₂ (69 mg; 0.11 mmol; 0.10 eq.), 1,4-dioxane (2.0 mL) and water (0.7 mL). RM is heated to 120° C. and irradiated with MW in the Biotage Initiator unit for 30 min. Purification by FCC (hexane/EtOAc; gradient). 5-(1H-1,3-Benzodiazol-6-yl)-7-chloroquinoxaline (100 mg; 0.37 mmol, yield 39%; 100% by UPLC) is obtained as a brownish-gray solid.

### Example 188

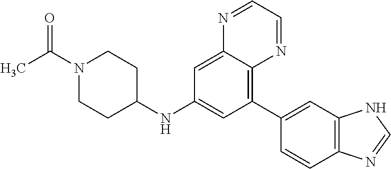 [SMILES: CC(=O)N1CCC(Nc2cc(-c3ccc4nc[nH]c4c3)c3nccnc3c2)CC1]

The product is prepared according to the modified General Procedure 2, described in Example 1 with 5-(1H-1,3-Benzodiazol-6-yl)-7-chloroquinoxaline (Intermediate 157) (50 mg; 0.18 mmol; 1.0 eq.), 1-(4-aminopiperidin-1-yl)ethan-1-one hydrochloride (31.8 mg; 0.18 mmol; 1.00 eq.), NaOtBu (68.5 mg; 0.71 mmol; 4.00 eq.), BINAP (22 mg; 0.04 mmol; 0.20 eq.), Pd₂(dba)₃ (16 mg; 0.02 mmol; 0.10 eq.) and [1,4]-dioxane (2.0 mL). Reaction is carried out in sealed tube at 120° C. for 18 h. RM is diluted with EtOAc and filtered through Celite® pad. Filtrate is evaporated to dynes and dissolved in water. pH of resulted mixture is adjusted to 7 with 1M HCl and extracted with n-butanol (3×10 mL). Combined extracts were dried over Na₂SO₄ and evaporated. Purification by preparative HPLC (ACN/0.1% aquas ammonia, gradient). 1-(4-{[8-(1H-1,3-benzodiazol-6-yl)quinoxalin-6-yl]amino}piperidin-1-yl)ethan-1-one (11.2 mg; 0.03 mmol; yield 15.3%; 94.1% by HPLC) is obtained as a yellow-greenish glass.

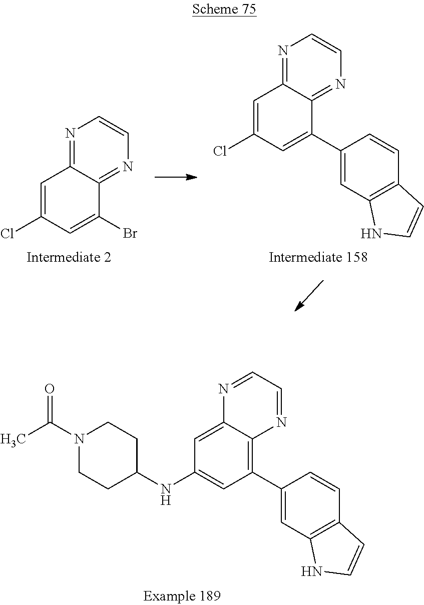 [SMILES: Clc1cc(Br)c2nccnc2c1 ; Clc1cc(-c2ccc3cc[nH]c3c2)c2nccnc2c1 ; CC(=O)N1CCC(Nc2cc(-c3ccc4cc[nH]c4c3)c3nccnc3c2)CC1]

### Intermediate 158

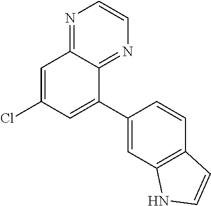 [SMILES: Clc1cc(-c2ccc3cc[nH]c3c2)c2nccnc2c1]

The product is prepared according to General Procedure 1, described for Intermediate 4 with 6-(tetramethyl-1,3,2-dioxaborolan-2-yl)-1H-indole (150.00 mg; 0.59 mmol; 1.00 eq.), 5-bromo-7-chloroquinoxaline (Intermediate 2) (292.00 mg; 1.19 mmol; 2.01 eq.), DIPEA (0.416 ml; 2.39 mmol; 4.04 eq.), Pd(dppf)Cl₂ (87 mg; 0.12 mmol; 0.20 eq.), 1,4-dioxane (2.500 ml) and water (2.500 ml). Reaction is carried out overnight at 85° C. with stirring. Then it is diluted with EtOAc and filtered through Celite®. The filtrate is washed with diluted HCl, water and brine. The organic layer is dried over anhydrous Na₂SO₄ and filtered. The filtrate is evaporated to dryness under reduced pressure and the residue is purified by FCC (hexane/EtOAc; gradient) to give 7-chloro-5-(1H-indol-6-yl)quinoxaline (148.00 mg; yield 89.1%; 99.8% by UPLC) as a yellow powder.

### Example 189

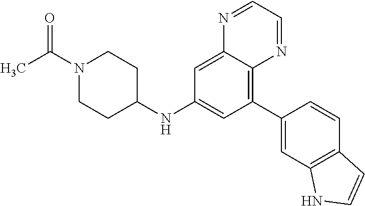 [SMILES: CC(=O)N1CCC(Nc2cc(-c3ccc4cc[nH]c4c3)c3nccnc3c2)CC1]

The product is prepared according to modified General Procedure 2, described in Example 1 with 7-chloro-5-(1H-indol-6-yl)quinoxaline (Intermediate 158) (120.00 mg; 0.43 mmol; 1.00 eq.), 1-(4-amino-piperidin-1-yl)-ethanone (121.76 mg; 0.86 mmol; 2.00 eq.), NaOtBu (144.01 mg; 1.50 mmol; 3.50 eq.), Pd₂(dba)₃ (39.21 mg; 0.04 mmol; 0.10 eq.), BINAP (59.32 mg; 0.1 mmol; 0.22 eq.) and 1,4-dioxane (2.000 ml). Reaction is carried out overnight at 100° C. with stirring. Then the mixture is diluted with EtOAc, DCM and filtered through Celite®. The sorbent is additionally washed with MeOH, DMF and acetone and the filtrate is evaporated to dryness under reduced pressure. The residue is purified by FCC (hexane/DCM; gradient then DCM/MeOH; gradient, NH₂-silica) and preparative HPLC then freeze-dried. The solid is dissolved in water then 2M NaOH is added and the product is extracted with DCM. The organic layer is washed with water and evaporated to dryness under reduced pressure to give 1-(4-{[8-(1H-indol-6-yl)quinoxalin-6-yl]amino}piperidin-1-yl)ethan-1-one (4.80 mg; yield 2.8%; 97.60% by HPLC) as a yellow powder.

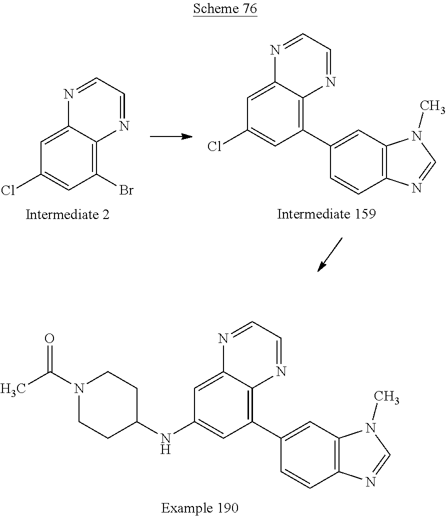 [SMILES: Clc1cc(Br)c2nccnc2c1 ; Cn1cnc2ccc(-c3cc(Cl)cc4nccnc34)cc21 ; CC(=O)N1CCC(Nc2cc(-c3ccc4ncn(C)c4c3)c3nccnc3c2)CC1]

### Intermediate 159

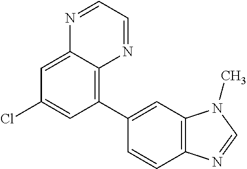 [SMILES: Cn1cnc2ccc(-c3cc(Cl)cc4nccnc34)cc21]

The product is prepared according to General Procedure, described for Intermediate 117 with 5-bromo-7-chloroquinoxaline (Intermediate 2) (0.28 g; 1.15 mmol; 1.00 eq.), (1-methyl-1H-1,3-benzodiazol-6-yl)boronic acid (245 mg; 1.26 mmol; 1.1 eq.), DIPEA (0.60 mL; 3.45 mmol; 3.00 eq.), Pd(dppf)Cl₂ (84 mg; 0.11 mmol; 0.10 eq.), 1,4-dioxane (2.4 mL) and water (0.8 mL). RM is heated to 120° C. and irradiated with MW in the Biotage Initiator unit for 30 min. Purification by crystallization from EtOAc. 7-chloro-5-(1-methyl-1H-1,3-benzodiazol-6-yl)quinoxaline (197 mg; 0.67 mmol, yield 58%; 99.6% by UPLC) is obtained as a greenish-yellow solid.

### Example 190

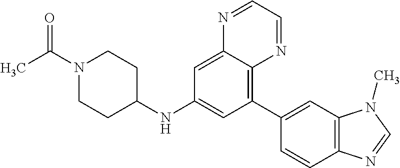 [SMILES: CC(=O)N1CCC(Nc2cc(-c3ccc4ncn(C)c4c3)c3nccnc3c2)CC1]

The product is prepared according to General Procedure 2, described in Example 1 with 7-chloro-5-(1-methyl-1H-1,3-benzodiazol-6-yl)quinoxaline (Intermediate 159) (50.00 mg; 0.17 mmol; 1.00 eq.), 1-(4-aminopiperidin-1-yl)ethan-1-one (39.4 mg; 0.22 mmol; 1.3 eq.), NaOtBu (65 mg; 0.68 mmol; 4.00 eq.), BINAP (21 mg; 0.03 mmol; 0.20 eq.), Pd₂(dba)₃ (15.5 mg; 0.02 mmol; 0.10 eq.) and toluene (1.00 mL). Reaction is carried out in sealed tube at 120° C. for 18 h. Purification by FCC (DCM/MeOH; gradient). 1-(4-{[8-(1-Methyl-1H-1,3-benzodiazol-6-yl)quinoxalin-6-yl]amino}piperidin-1-yl)ethan-1-one (18.6 mg; 0.04 mmol; yield 26%; 95.5% by HPLC) is obtained as a green-yellow glass.

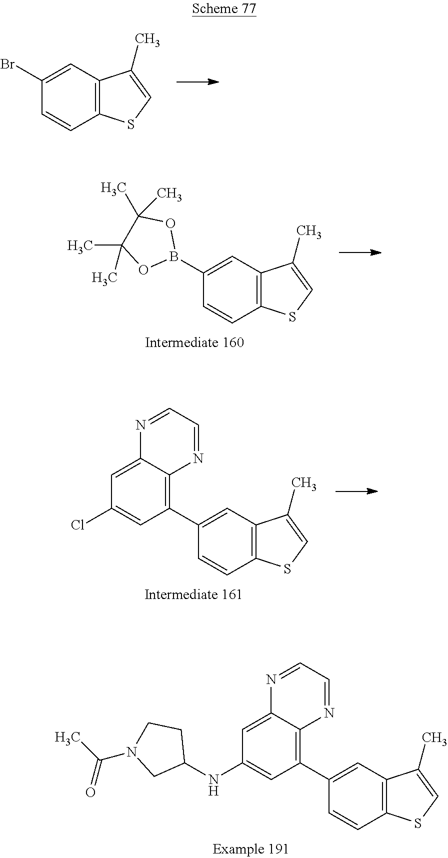 [SMILES: Cc1csc2ccc(Br)cc12 ; Cc1csc2ccc(B3OC(C)(C)C(C)(C)O3)cc12 ; Cc1csc2ccc(-c3cc(Cl)cc4nccnc34)cc12 ; CC(=O)N1CCC(Nc2cc(-c3ccc4scc(C)c4c3)c3nccnc3c2)C1]

### Intermediate 160

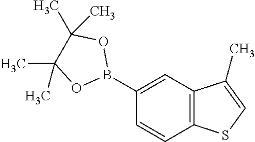 [SMILES: Cc1csc2ccc(B3OC(C)(C)C(C)(C)O3)cc12]

The product is prepared according to General Procedure 51, described for Intermediate 79 with 5-5-bromo-3-methyl-1-benzothiophene (0.50 g; 2.20 mmol; 1.00 eq.), 4,4,5,5-tetramethyl-2-(tetramethyl-1,3,2-dioxaborolan-2-yl)-1,3,2-dioxaborolane (726.75 mg; 2.86 mmol; 1.3 eq.), KOAc (432.11 mg; 4.40 mmol; 2.0 eq.) and [1,4]-Dioxane (5.00 ml). Resulted slurry is flashed with argon and Pd(dppf)Cl₂ (161.08 mg; 0.22 mmol; 0.10 eq.) is added under argon atmosphere. Reaction is carried out for 18 h at 100° C. Purification by FCC (Hexane/EtOAc, gradient). 4,4,5,5-Tetramethyl-2-(3-methyl-1-benzothiophen-5-yl)-1,3,2-dioxaborolane (536 mg; 1.91 mmol; yield 86.8%; 97.7% by UPLC) is obtained as colorless solid.

### Intermediate 161

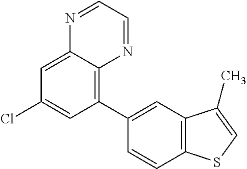 [SMILES: Cc1csc2ccc(-c3cc(Cl)cc4nccnc34)cc12]

Product is prepared according to General Procedure 54, described for Intermediate 117 with 5-bromo-7-chloroquinoxaline (Intermediate 2) (200.0 mg; 0.81 mmol; 1.00 eq.), 4,4,5,5-tetramethyl-2-(3-methyl-1-benzothiophen-5-yl)-1,3,2-dioxaborolane (Intermediate 160) (265.7 mg; 0.97 mmol; 1.2 eq.), DIPEA (0.28 ml; 1.61 mmol; 2.00 eq.), Pd(dppf)Cl₂ (59 mg; 0.08 mmol; 0.10 eq.), water (1 mL) and [1,4]-dioxane (2 mL). RM is heated to 120° C. and irradiated with MW in the Biotage Initiator unit for 30 min. Purification by FCC (hexane/EtOAc; gradient). 7-Chloro-5-(3-methyl-1-benzothiophen-5-yl)quinoxaline (222.0 mg; 0.64 mmol; yield 78.7%; 89% by UPLC) is obtained as white crystals.

### Example 191

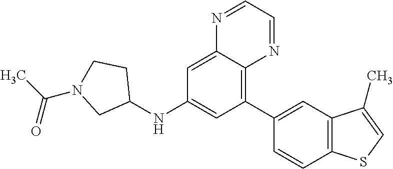 [SMILES: CC(=O)N1CCC(Nc2cc(-c3ccc4scc(C)c4c3)c3nccnc3c2)C1]

The product is prepared according to the modified General Procedure 2, described in Example 1 with 7-Chloro-5-(3-methyl-1-benzothiophen-5-yl)quinoxaline (Intermediate 161) (70 mg; 0.20 mmol; 1 eq), 1-(3-aminopyrrolidin-1-yl)ethan-1-one (30.8 mg; 0.24 mmol; 1.2 eq.), NaOtBu (38.5 mg; 0.40 mmol; 2.00 eq.), BINAP (25 mg; 0.04 mmol; 0.20 eq.), Pd₂(dba)₃ (18.3 mg; 0.02 mmol; 0.10 eq.) and [1,4]-Dioxane (2.0 ml). Reaction is carried out in sealed tube at 110° C. for 18 h. Purification by preparative HPLC (ACN/0.1% aquas ammonia, gradient). 1-(3-{[8-(3-Methyl-1-benzothiophen-5-yl)quinoxalin-6-yl]amino}pyrrolidin-1-yl)ethan-1-one (32.5 mg; 0.08 mmol; yield 40.1%; 99.5% by HPLC) is obtained as yellow-brownish solid.

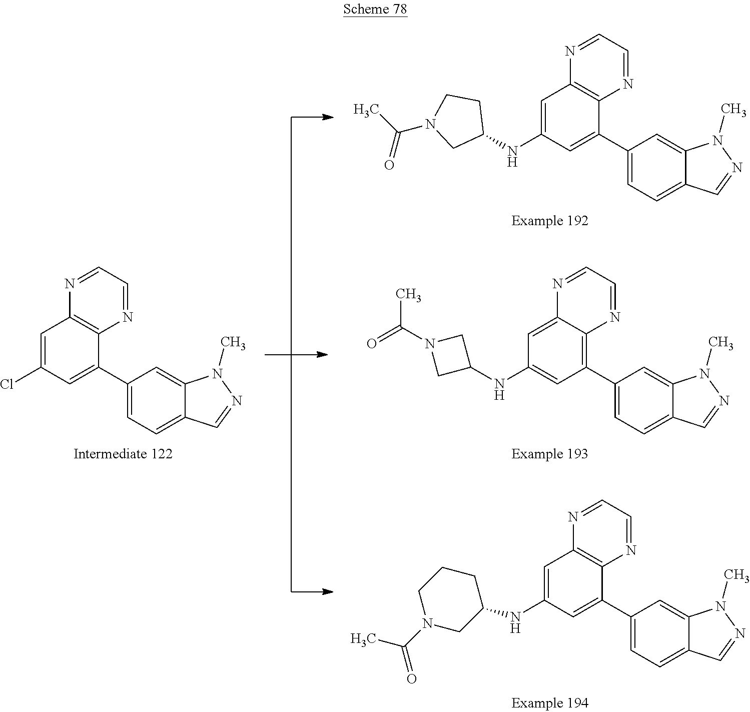 [SMILES: CC(=O)N1CC[C@H](Nc2cc(-c3ccc4cnn(C)c4c3)c3nccnc3c2)C1 ; Cn1ncc2ccc(-c3cc(Cl)cc4nccnc34)cc21 ; CC(=O)N1CC(Nc2cc(-c3ccc4cnn(C)c4c3)c3nccnc3c2)C1 ; CC(=O)N1CCC[C@H](Nc2cc(-c3ccc4cnn(C)c4c3)c3nccnc3c2)C1]

### Example 192

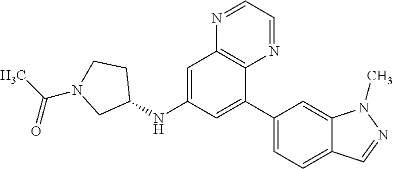 [SMILES: CC(=O)N1CC[C@H](Nc2cc(-c3ccc4cnn(C)c4c3)c3nccnc3c2)C1]

The product is prepared according to General Procedure 2, described in Example 1 with 7-chloro-5-(1-methyl-1H-indazol-6-yl)quinoxaline (Intermediate 122) (40.00 mg; 0.14 mmol; 1.00 eq.), 1-[(3S)-3-aminopyrrolidin-1-yl]ethan-1-one hydrochloride (56.30 mg; 0.34 mmol; 2.52 eq.), NaOtBu (52.17 mg; 0.54 mmol; 4.00 eq.), BINAP (16.90 mg; 0.03 mmol; 0.20 eq.), Pd₂(dba)₃ (12.43 mg; 0.01 mmol; 0.10 eq.) and toluene (1.50 mL). Reaction is carried out in sealed tube at 120° C. for 24 h. Purification by FCC (hexane/EtOAc; gradient). Repurification by preparative HPLC is done. 1-[(3S)-3-{[8-(1-methyl-1H-indazol-6-yl)quinoxalin-6-yl]amino}pyrrolidin-1-yl]ethan-1-one (7.90 mg; yield 14.2%; 94.5% by HPLC) is obtained as a green powder.

### Example 193

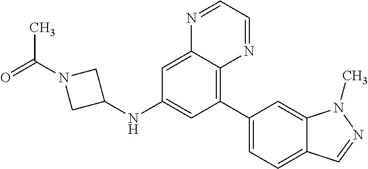 [SMILES: CC(=O)N1CC(Nc2cc(-c3ccc4cnn(C)c4c3)c3nccnc3c2)C1]

The product is prepared according to General Procedure 2, described in Example 1 with 7-chloro-5-(1-methyl-1H-indazol-6-yl)quinoxaline (Intermediate 122) (42.00 mg; 0.14 mmol; 1.00 eq.), 1-(3-aminoazetidin-1-yl)-ethan-1-one hydrochloride (42.92 mg; 0.28 mmol; 2.00 eq.), NaOtBu (54.78 mg; 0.57 mmol; 4.00 eq.), BINAP (17.75 mg; 0.03 mmol; 0.20 eq.), Pd₂(dba)₃ (13.05 mg; 0.01 mmol; 0.10 eq.) and toluene (1.26 mL). Reaction is carried out in sealed tube at 120° C. for 24 h. Purification by FCC (EtOAc/Methanol; gradient) 1-(3-{[8-(1-methyl-1H-indazol-6-yl)quinoxalin-6-yl]amino}azetidin-1-yl)ethan-1-one (13.80 mg; yield 25.6%; 98.3% by HPLC) is obtained as a green powder.

### Example 194

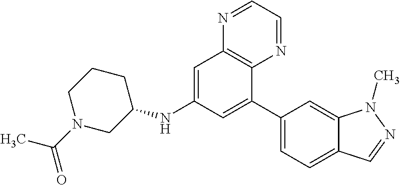 [SMILES: CC(=O)N1CCC[C@H](Nc2cc(-c3ccc4cnn(C)c4c3)c3nccnc3c2)C1]

The product is prepared according to General Procedure 2, described in Example 1 with 7-chloro-5-(1-methyl-1H-indazol-6-yl)quinoxaline (Intermediate 122) (40.00 mg; 0.14 mmol; 1.00 eq.), 1-[(3S)-(3-aminopiperidin-1-yl)]ethan-1-one hydrochloride (61.10 mg; 0.34 mmol; 2.52 eq.), NaOtBu (52.17 mg; 0.54 mmol; 4.00 eq.), BINAP (16.90 mg; 0.03 mmol; 0.20 eq.), Pd₂(dba)₃ (12.43 mg; 0.01 mmol; 0.10 eq.) and toluene (1.50 mL). Reaction is carried out in sealed tube at 120° C. for 24 h. Purification by FCC (hexane/EtOAc; gradient) followed by preparative HPLC repurification gives 1-[(3S)-3-{[8-(1-methyl-1H-indazol-6-yl)quinoxalin-6-yl]amino}piperidin-1-yl]ethan-1-one (10.10 mg; yield 18.5%; 99.6% by HPLC) is obtained as a green powder.

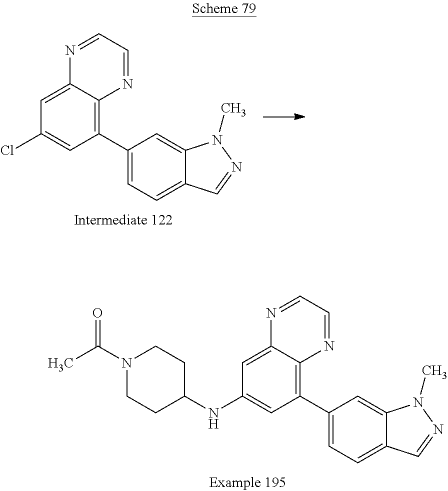 [SMILES: Cn1ncc2ccc(-c3cc(Cl)cc4nccnc34)cc21 ; CC(=O)N1CCC(Nc2cc(-c3ccc4cnn(C)c4c3)c3nccnc3c2)CC1]

### Example 195

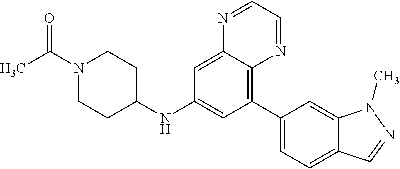 [SMILES: CC(=O)N1CCC(Nc2cc(-c3ccc4cnn(C)c4c3)c3nccnc3c2)CC1]

The product is prepared according to General Procedure 2, described in Example 1 with 1-(4-Aminopiperidin-1-yl)ethan-1-one hydrochloride (72.47 mg; 0.41 mmol; 2.00 eq.), NaOtBu (77.97 mg; 0.81 mmol; 4.00 eq.), Pd₂(dba)₃ (18.57 mg; 0.02 mmol; 0.10 eq.), BINAP (25.26 mg; 0.04 mmol; 0.20 eq.), 7-chloro-5-(1-methyl-1H-indazol-6-yl)quinoxaline (Intermediate 122) (70.00 mg; 0.20 mmol; 1.00 eq.) and toluene (2.00 ml). Reaction is carried out overnight at 120° C. Then RM is diluted with AcOEt, filtered thorough Celite® and evaporated. The residue is purified by FCC (DCM/MeOH; gradient) to give 1-(4-{[8-(1-methyl-1H-indazol-6-yl)quinoxalin-6-yl]amino}piperidin-1-yl)ethan-1-one (58.30 mg; yield 71.5%; 99.6% by HPLC) as a green yellow powder.

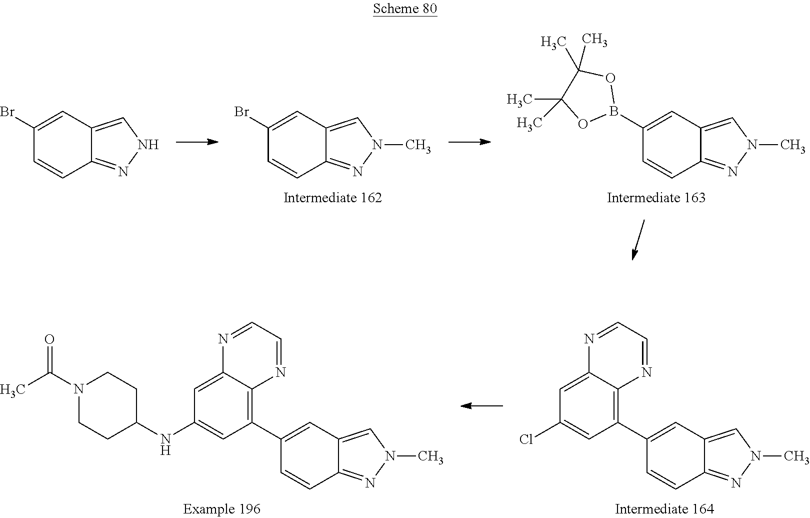

### Intermediate 162

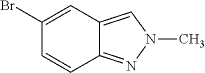

The product is prepared according to General Procedure 40, described for Intermediate 38 with 5-bromo-1H-indazole (1.00 g; 4.97 mmol; 1.00 eq.), NaH (60% in mineral oil), iodomethane (0.40 ml; 6.47 mmol; 1.30 eq.) and THF (50.00 ml). RM is stirred overnight at rt. Purification by FCC (hexane/EtOAc; gradient) to give 5-bromo-2-methyl-2H-indazole (407.00 mg; yield 38.6%; 99.5% by UPLC) as a yellow solid.

### Intermediate 163

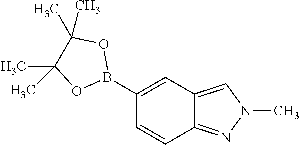

Product is prepared according to General Procedure 51, described for Intermediate 79 with 5-Bromo-2-methyl-2H-indazole (Intermediate 162) (397.00 mg; 1.87 mmol; 1.00 eq.), bis(pinacolato)diboron (617.85 mg; 2.43 mmol; 1.30 eq.), KOAc (367.36 mg; 3.74 mmol; 2.00 eq.), Pd(dppf)Cl₂ (14.00 mg; 0.02 mmol; 0.01 eq.) and 1,4-dioxane (5.00 ml). Reaction is carried out overnight at 100° C. with stirring. Purification by FCC (hexane/EtOAc; gradient) to give 2-methyl-5-(tetramethyl-1,3,2-dioxaborolan-2-yl)-2H-indazole (362.90 mg; yield 72.7%; 96.8% by UPLC) as a white solid.

### Intermediate 164

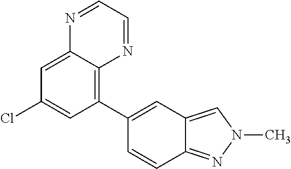 [SMILES: Cn1cc2cc(-c3cc(Cl)cc4nccnc34)ccc2n1]

The product is prepared according to General Procedure 1, described for Intermediate 4 with 2-Methyl-5-(tetramethyl-1,3,2-dioxaborolan-2-yl)-2H-indazole (Intermediate 163) (355.00 mg; 1.33 mmol; 1.00 eq.), 5-bromo-7-chloroquinoxaline (Intermediate 2) (325.78 mg; 1.33 mmol; 1.00 eq.), DIPEA (0.464 ml; 2.66 mmol; 2.00 eq.), Pd(dppf)Cl₂ (97.4 mg; 0.13 mmol; 0.10 eq.), 1,4-dioxane (2.50 ml) and water (2.50 ml). Reaction is carried out for 3 h at 85-90° C. Then it is diluted with EtOAc and filtered through Celite®. The filtrate is evaporated under reduced pressure and the residue is purified by FCC (hexane->EtOAc; gradient then EtOAc/MeOH; gradient) to give 7-chloro-5-(2-methyl-2H-indazol-5-yl)quinoxaline (276.00 mg; yield 70.3%; 100.0% by UPLC) as a yellow powder.

### Example 196

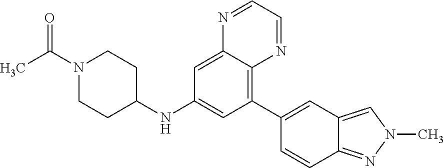 [SMILES: CC(=O)N1CCC(Nc2cc(-c3ccc4nn(C)cc4c3)c3nccnc3c2)CC1]

The product is prepared according to General Procedure 2, described in Example 1 with 1-(4-Aminopiperidin-1-yl)ethan-1-one (85.00 mg; 0.60 mmol; 2.52 eq.), Pd₂(dba)₃ (21.75 mg; 0.02 mmol; 0.10 eq.), NaOtBu (91.30 mg; 0.95 mmol; 4.00 eq.), BINAP (29.58 mg; 0.05 mmol; 0.20 eq.) and 7-chloro-5-(2-methyl-2H-indazol-5-yl)quinoxaline (Intermediate 164) (70.00 mg; 0.24 mmol; 1.00 eq.) and toluene (2.00 ml). Reaction is carried out for 3 h at 120° C. Then RM is diluted with AcOEt, filtered thorough Celite® and evaporated under reduced pressure. The residue is purified by FCC (DCM/MeOH; gradient) to give 1-(4-{[8-(2-methyl-2H-indazol-5-yl)quinoxalin-6-yl]amino}piperidin-1-yl)ethan-1-one (60.60 mg; yield 63.1%; 99.0% by HPLC) as a green yellow sticky powder.

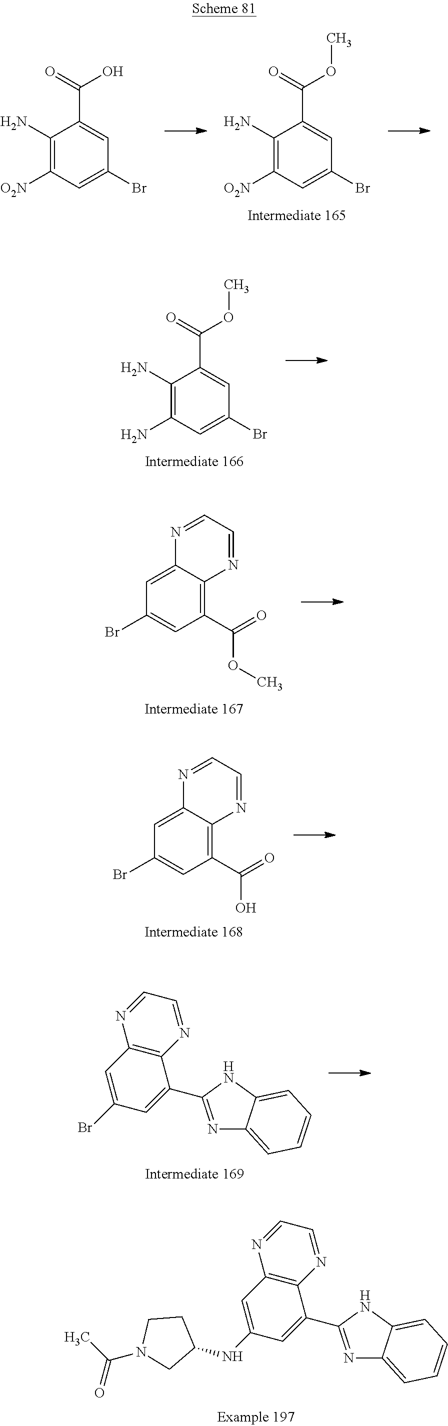

### Intermediate 167

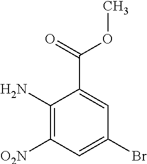

Roundbottom flask equipped with reflux condenser is charged with 2-Amino-5-bromo-3-nitro-benzoic acid (1.00 g; 3.64 mmol; 1.00 eq.) and anhydrous methanol (20.00 ml). The Thionyl chloride (0.53 ml; 7.28 mmol; 2.00 eq.) is added dropwise under ambient temperature. RM is refluxed for 18 h and then cooled down to room temperature and second portion of thionyl chloride is added (0.53 ml; 7.28 mmol; 2.00 eq.). RM is refluxed for additional 18 h. Then the RM is cooled down to room temperature. Methanol is evaporated in vacuo to give light yellow solid. Resulted solid is dissolved in ethyl acetate and washed successively with 1M HCl, water and brine. Organic extract is dried over Na₂SO₄ and evaporated. Crude product is purified by FCC (Silica, EtOAc/hexane gradient) to give methyl 2-amino-5-bromo-3-nitrobenzoate (0.86 g; 3.02 mmol; yield 83.0%; 97% by UPLC) as yellow needles.

### Intermediate 166

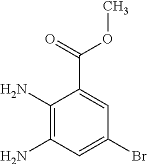

Roundbottom flask equipped with reflux condenser is charged with methyl 2-amino-5-bromo-3-nitrobenzoate (Intermediate 165) (0.86 g; 3.02 mmol; 1.00 eq.), iron (1.01 g; 18.13 mmol; 6.00 eq.) and ammonium chloride (1.62 g; 30.22 mmol; 10.00 eq.). Resulted slurry is stirred and refluxed for 4 h, then cooled down to room temperature, diluted with EtOAc and phased by pad of celite. Filtrate is extracted subsequently with water, and brine. Organic layer id dried over Na₂SO₄ and evaporated to give methyl 2,3-diamino-5-bromobenzoate (0.71 g; 2.69 mmol; yield 89.1%; 93% by UPLC) as brown solid which was used in next step without further purification.

### Intermediate 167

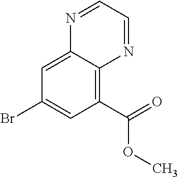

Intermediate 167 methyl 7-bromoquinoxaline-5-carboxylate is prepared according to procedure described for Intermediate 1 with methyl 2,3-diamino-5-bromobenzoate (Intermediate 166) (0.71 g; 2.69 mmol; 1.00 eq.), ethanol (50.00 ml) and the [1,4]-dioxane-2,3-diol (485.39 mg; 4.04 mmol; 1.50 eq.). Purification by FCC (silica, EtOAc gradient in hexane) gives methyl 7-bromoquinoxaline-5-carboxylate (0.54 g; 2.00 mmol; yield 74.2%, 98.9 by UPLC) as pink-gray solid.

### Intermediate 168

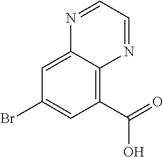 [SMILES: O=C(O)c1cc(Br)cc2nccnc12]

Roundbottom flask equipped with reflux condenser is charged with gives methyl 7-bromoquinoxaline-5-carboxylate (Intermediate 167) (54.00 mg; 2.04 mmol; 1.00 eq.), methanol (15.00 ml) and 5M NaOH (aq) (15.00 ml; 75 mmol, 38 eq). RM is heated to 50° C. with stirring for 2 h. Then RM is acidifying with 10 M HCl (7 mL), and mixture is stirred for 30 min. Then resulted slurry is extracted 4 times with DCM. Organic layer is raised with water and brine, dried with Na₂SO₄ and solvent is evaporated to give 7-Bromo-quinoxaline-5-carboxylic acid (517.00 mg; 2.04 mmol; yield 100%; 10% by UPLC) as colorless solid.

### Intermediate 169

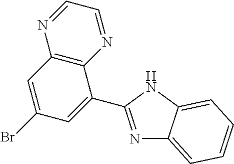 [SMILES: Brc1cc(-c2nc3ccccc3[nH]2)c2nccnc2c1]

A sealed tube charged with 7-Bromo-quinoxaline-5-carboxylic acid (Intermediate 168), benzene-1,2-diamine (44.87 mg; 0.41 mmol; 1.05 eq.) and polyphosphoric acid (2.00 mL) is stirred at 100° C. for 24h. After this time RM is cooled down to room temperature and is diluted with water. Then pH is adjusted to 6 using 5M aqueous solution of NaOH and mixture is extracted with EtOAc to afford 5-(1H-1,3-benzodiazol-2-yl)-7-chloroquinoxaline (23.80 mg; yield 16.5%; 89.1% by UPLC) as brown solid.

### Example 197

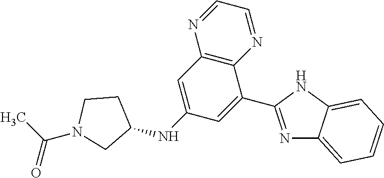 [SMILES: CC(=O)N1CC[C@H](Nc2cc(-c3nc4ccccc4[nH]3)c3nccnc3c2)C1]

The product is prepared according to General Procedure 2, described in Example 1 with 5-(1H-1,3-benzodiazol-2-yl)-7-chloroquinoxaline (Intermediate 169) (20.00 mg; 0.07 mmol; 1.00 eq.), 1-[(3S)-3-aminopyrrolidin-1-yl]ethan-1-one hydrochloride (27.40 mg; 0.21 mmol; 3.00 eq.), NaOtBu (27.39 mg; 0.28 mmol; 4.00 eq.), BINAP (8.87 mg; 0.01 mmol; 0.20 eq.), Pd₂(dba)₃ (6.52 mg; 0.01 mmol; 0.10 eq.) and toluene (1.00 mL). Reaction is carried out in sealed tube at 120° C. for 24 h. Purification by FCC (DCM/MeOH; gradient). Repurification by preparative HPLC is done. 1-[(3S)-3-{[8-(1H-1,3-benzodiazol-2-yl)quinoxalin-6-yl]amino}pyrrolidin-1-yl]ethan-1-one (1.8 mg; yield 6.3%; 93.4% by HPLC) is obtained as a yellow powder.

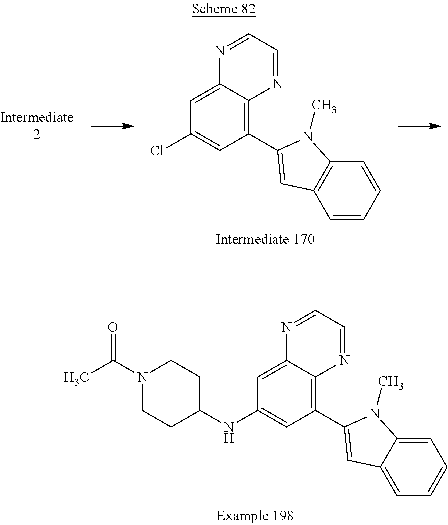 [SMILES: Cn1c(-c2cc(Cl)cc3nccnc23)cc2ccccc21 ; CC(=O)N1CCC(Nc2cc(-c3cc4ccccc4n3C)c3nccnc3c2)CC1]

### Intermediate 170

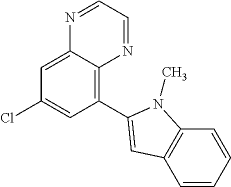 [SMILES: Cn1c(-c2cc(Cl)cc3nccnc23)cc2ccccc21]

Intermediate 170 7-chloro-5-(1-methyl-1H-indol-2-yl)quinoxaline is prepared by modification of General Procedure 1, described for Intermediate 4 with 5-Bromo-7-chloroquinoxaline (Intermediate 2) (400.00 mg; 1.61 mmol; 1.00 eq.), 1-methyl-2-(tetramethyl-1,3,2-dioxaborolan-2-yl)-1H-indole (461 mg; 1.78 mmol; 1.10 eq.), DIPEA (0.84 ml; 4.84 mmol; 3.00 eq.), Pd(dppf)Cl₂ (118 mg; 0.16 mmol; 0.10 eq.), [1,4]-dioxane (3.4 ml) and water (1.2 ml). RM is stirred overnight in an oil bath preheated to 100° C. RM is workuped accordingly to General procedure 1. Purification by FCC (silica, gradient of EtOAc in hexane) gives 7-chloro-5-(1-methyl-1H-indol-2-yl)quinoxaline (260.00 mg; 0.87 mmol; yield 53.7%; 98% by UPLC) as yellow crystals.

### Example 198

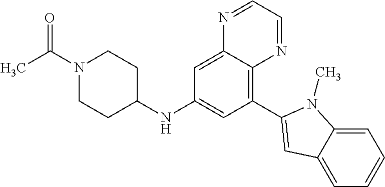 [SMILES: CC(=O)N1CCC(Nc2cc(-c3cc4ccccc4n3C)c3nccnc3c2)CC1]

The product is prepared according to General Procedure 2, described in Example 1 with 7-chloro-5-(1-methyl-1H-indol-2-yl)quinoxaline (40.00 mg; 0.14 mmol; 1.00 eq.) (Intermediate 170), 1-(4-aminopiperidin-1-yl)ethan-1-one (38.73 mg; 0.27 mmol; 2.00 eq.), NaOtBu (52.35 mg; 0.54 mmol; 4.00 eq.), BINAP (16.96 mg; 0.03 mmol; 0.20 eq.), Pd₂(dba)₃ (12.47 mg; 0.01 mmol; 0.10 eq.) and toluene (1.50 mL). Reaction is carried out in sealed tube at 120° C. for 24 h. Purification by FCC (dichloromethane/methanol; gradient). 1-(4-{[8-(1-methyl-1H-indol-2-yl)quinoxalin-6-yl]amino}piperidin-1-yl)ethan-1-one (17.00 mg; yield 30.3%; 97.0% by HPLC) is obtained as a yellow powder.

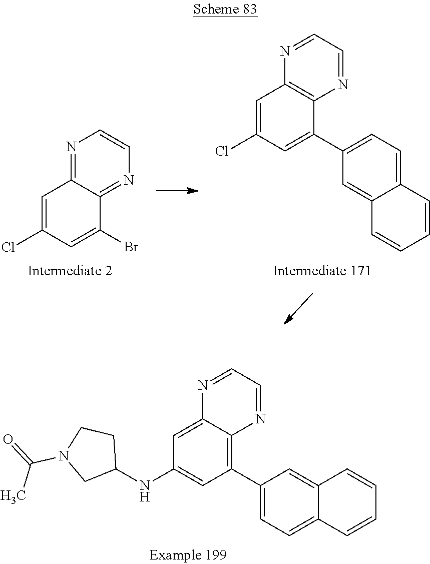 [SMILES: Clc1cc(Br)c2nccnc2c1 ; Clc1cc(-c2ccc3ccccc3c2)c2nccnc2c1 ; CC(=O)N1CCC(Nc2cc(-c3ccc4ccccc4c3)c3nccnc3c2)C1]

### Intermediate 171

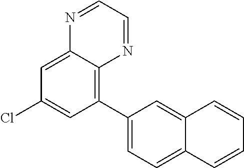 [SMILES: Clc1cc(-c2ccc3ccccc3c2)c2nccnc2c1]

The product is prepared according to modified General Procedure 1, described for Intermediate 4 with 5-bromo-7-chloroquinoxaline (Intermediate 2) (200.00 mg; 0.82 mmol; 1.00 eq.), (naphthalen-2-yl)boronic acid (154.62 mg; 0.90 mmol; 1.10 eq.), Pd(dppf)Cl2 (59.78 mg; 0.08 mmol; 0.10 eq.), DIPEA (0.427 ml; 2.45 mmol; 3.00 eq.), 1,4-dioxane (7.000 ml) and water (3.000 ml). Reaction is carried out at MW 120° C. for 1 h. Then it is diluted with EtOAc, DCM and filtered through Celite®. The filtrate is evaporated under reduced pressure and the residue is purified by FCC (hexane/DCM; gradient) to give 7-chloro-5-(naphthalen-2-yl)quinoxaline (154.80 mg; yield 64.7%; 99.30% by UPLC) as a white powder.

### Example 199

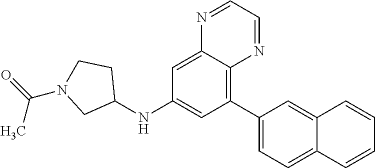 [SMILES: CC(=O)N1CCC(Nc2cc(-c3ccc4ccccc4c3)c3nccnc3c2)C1]

The product is prepared according to General Procedure 2, described in Example 1 with 7-chloro-5-(naphthalen-2-yl)quinoxaline (Intermediate 171) (48.00 mg; 0.16 mmol; 1.00 eq.), 1-(3-Amino-pyrrolidin-1-yl)-ethanone (63.04 mg; 0.49 mmol; 3.00 eq.), Pd₂(dba)₃ (15.01 mg; 0.02 mmol; 0.10 eq.), NaOtBu (20.00 mg; 0.21 mmol; 1.27 eq.) BINAP (20.42 mg; 0.03 mmol; 0.20 eq.) and toluene (1.500 ml). Reaction is carried out overnight at 110° C. Then it is diluted with EtOAc, DCM and filtered through Celite®. The filtrate is evaporated under reduced pressure and the residue is purified by FCC (DCM/MeOH; gradient) to give 1-(3-{[8-(naphthalen-2-yl)quinoxalin-6-yl]amino}pyrrolidin-1-yl)ethan-1-one (39.70 mg; yield 62.9%; 99.40% by HPLC) as a yellow green powder.

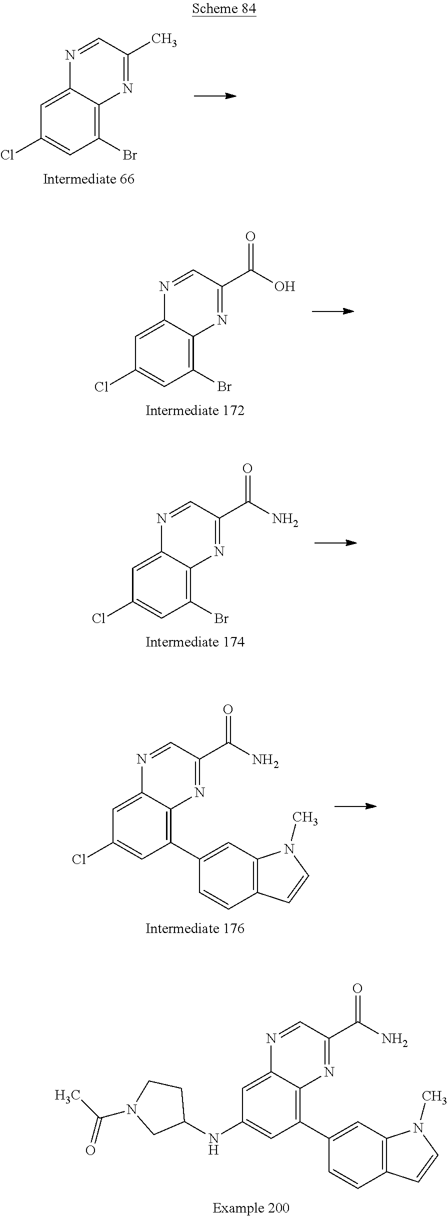 [SMILES: Cc1cnc2cc(Cl)cc(Br)c2n1 ; O=C(O)c1cnc2cc(Cl)cc(Br)c2n1 ; NC(=O)c1cnc2cc(Cl)cc(Br)c2n1 ; Cn1ccc2ccc(-c3cc(Cl)cc4ncc(C(N)=O)nc34)cc21 ; CC(=O)N1CCC(Nc2cc(-c3ccc4ccn(C)c4c3)c3nc(C(N)=O)cnc3c2)C1]

### Intermediate 172 & 173

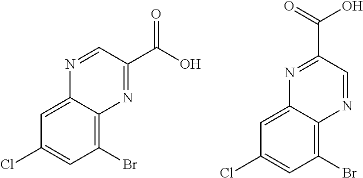 [SMILES: O=C(O)c1cnc2cc(Cl)cc(Br)c2n1 ; O=C(O)c1cnc2c(Br)cc(Cl)cc2n1]

The three necked roundbottom flask equipped with reflux condenser, argon inlet, the is charged with selenium(IV) oxide (2.82 g; 25.39 mmol; 3.00 eq.) and [1,4]-dioxane (15 ml). The flask content is flashed with argon and stirred with heating to reflux. Solution of 8-bromo-6-chloro-2-methylquinoxaline & 5-bromo-7-chloro-2-methylquinoxaline (Intermediates 65&66) (2.87 g; 11.0 mmol; 4:1 mol/mol by UPLC) (Prepared according to the General procedure 47 for Intermediates 65 & 66 and used as crude isomers separation) in [1,4]-dioxane (15 mL) is added dropwise to the refluxed slurry of Selenium(IV) oxide. The RM is refluxed for 18 h and then cooled to rt. RM is diluted with EtOAc and filtered through Celite® pad. Filtrate is treated with 60 mL of 1M NaOH. Formed precipitate is collected by filtration and washed successively with EtOAc, hexane and dried over air to give crude mixture of 8-bromo-6-chloroquinoxaline-2-carboxylic acid (Intermediate 172) & 5-bromo-7-chloro-quinoxaline-2-carboxylic acid (Intermediate 173) as sodium salt (1.51 g, 4.88 mmol, total yield 44.4%, 100% by UPLC; 4:1 ratio basing on the isomers ratio from starting material).

### Intermediate 174 & 175

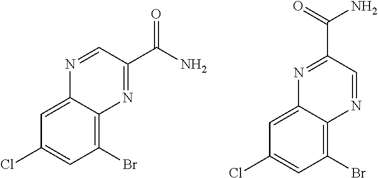 [SMILES: NC(=O)c1cnc2cc(Cl)cc(Br)c2n1 ; NC(=O)c1cnc2c(Br)cc(Cl)cc2n1]

### Stage 1.

Crude mixture of sodium 8-bromo-6-chloroquinoxaline-2-carboxylate (Intermediate 172) & sodium 5-bromo-7-chloroquinoxaline-2-carboxylate (Intermediate 173) (0.50 g; 1.62 mmol; 4:1 isomers ratio) is suspended in Methanol (30.00 ml). Resulted slurry is acidified with 1M HCl to obtain clear solution. Methanol is evaporated on rotavap and remaining water solution is extracted with EtOAc (3×25 mL). Combined organic layers are washed with water and brine, dried over Na₂SO₄ and evaporated. Crude mixture of 8-bromo-6-chloroquinoxaline-2-carboxylic acid and 5-bromo-7-chloroquinoxaline-2-carboxylic acid (0.394 g) is used in next ntext step as crude product.

### Stage 2.

The 4-Methylmorpholine (0.43 ml; 3.88 mmol; 3.00 eq.) and 2-chloro-4,6-dimethoxy-1,3,5-triazine (681 mg; 3.88 mmol; 3.00 eq.) are added to the stirred anhydrous THF (1.00 ml). Resulted mixture is stirred for 15 min to obtain white precipitate of DMT-MM. The slurry of DMT-MM is diluted with anhydrous DMF (5.00 ml) and crude product from Step 1 is added in one portion followed by addition of ammonia solution 0.5 M in dioxane (26 ml; 13 mmol; 10.00 eq.) addition. Resulted mixture is stirred at rt for 16 h. Then RM is diluted in EtOAc (25 ml) and washed brine (3×10 ml). Organic layer is evaporated in vacuo. Remaining residue is crystallized from MeOH. There was obtained mixture of 8-bromo-6-chloroquinoxaline-2-carboxamide and 5-bromo-7-chloroquinoxaline-2-carboxamide (0.35 g; 1.22 mmol; total yield 75.4%; in 4:1 molar ratio (based on isomers ratio from condensation step) as beige solid.

### Intermediate 176

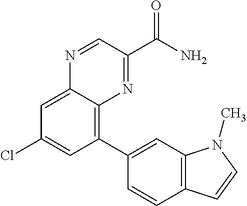 [SMILES: Cn1ccc2ccc(-c3cc(Cl)cc4ncc(C(N)=O)nc34)cc21]

The product is prepared according to modified General procedure for Suzuki-Miyaura cross couplings under MW conditions, described for Intermediate 154 with 8-bromo-6-chloroquinoxaline-2-carboxamide (Intermediate 174) (150 mg; 0.42 mmol; 1.00 eq.), 1-methyl-6-(tetramethyl-1,3,2-dioxaborolan-2-yl)-1H-indole (143.2 mg; 0.55 mmol; 1.3 eq.), cesium carbonate (345.4 mg; 1.06 mmol; 2.5 eq.), Pd(dppf)Cl₂ (31 mg; 0.04 mmol; 0.10 eq.), water (0.3 ml) and [1,4]-dioxane (1.5 ml). RM is heated to 120° C. and irradiated with MW in the Biotage Initiator unit for 30 min. RM is filtrated by Celite® pad and evaporated to give crude 6-chloro-8-(1-methyl-1H-indol-6-yl)quinoxaline-2-carboxamide (240 mg; 0.40 mmol; yield 56% by UPLC) brown solid.

### Example 200

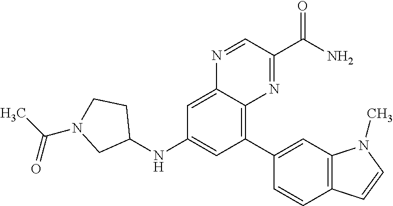 [SMILES: CC(=O)N1CCC(Nc2cc(-c3ccc4ccn(C)c4c3)c3nc(C(N)=O)cnc3c2)C1]

The product is prepared according to the modified General Procedure 2, described in Example 1 with 6-chloro-8-(1-methyl-1H-indol-6-yl)quinoxaline-2-carboxamide (Intermediate 176) (240 mg; 0.40 mmol; 1 eq), 1-[3-amino-pyrrolidin-1-yl]ethan-1-one (61.4 mg; 0.48 mmol; 1.2 eq.), NaOtBu (59 mg; 0.61 mmol; 4.00 eq.), BINAP (49.7 mg; 0.8 mmol; 0.2 eq.), Pd₂(dba)₃ (36.5 mg; 0.04 mmol; 0.10 eq.) and [1,4]-dioxane (2.4 ml). Reaction is carried out in sealed tube at 120° C. for 18 h. Purification by FCC (MeOH/DCM, gradient). Repurification by preparative HPLC (ACN/0.5% FA). 6-[(1-Acetylpyrrolidin-3-yl)amino]-8-(1-methyl-1H-indol-6-yl)quinoxaline-2-carboxamide (12.3 mg; 0.03 mmol; yield 7.1%; 99.4% by HPLC) is obtained as white solid.

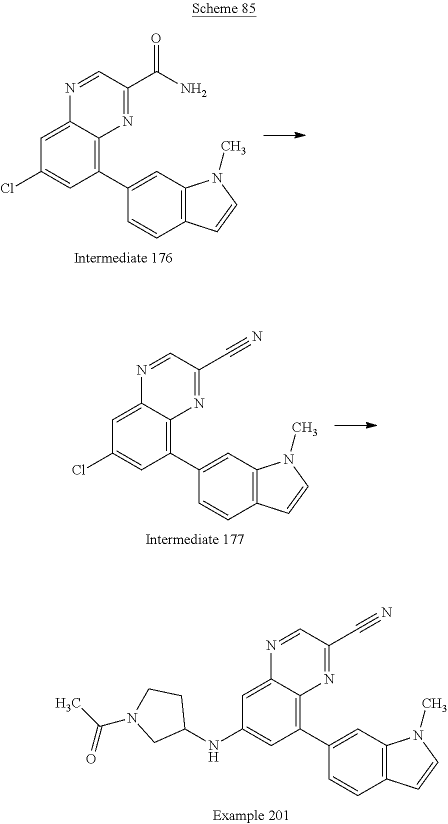 [SMILES: Cn1ccc2ccc(-c3cc(Cl)cc4ncc(C(N)=O)nc34)cc21 ; Cn1ccc2ccc(-c3cc(Cl)cc4ncc(C#N)nc34)cc21 ; CC(=O)N1CCC(Nc2cc(-c3ccc4ccn(C)c4c3)c3nc(C#N)cnc3c2)C1]

### Intermediate 177

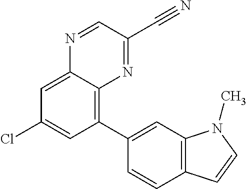 [SMILES: Cn1ccc2ccc(-c3cc(Cl)cc4ncc(C#N)nc34)cc21]

The two necked roundbottom flask equipped with reflux condenser septum is charged with To the mixture of 6-chloro-8-(1-methyl-1H-indol-6-yl)quinoxaline-2-carboxamide (Intermediate 176) (0.20 g; 0.52 mmol; 1.00 eq.) and anhydrous Acetonitrile (20.00 ml). The POCl₃ (0.1 ml; 1.04 mmol; 2.0 eq) is added dropwise at rt. Resulted mixture is refluxed for 2 h. Then RM is poured to 300 ml of water and resulted complex mixture is filtered by tine pad of Celite®. Filtrate is extracted with DCM (3×25 mL). Organic layers are combined and successively washed with water, saturated NaHCO₃, brine, dried over Na₂SO₄ and evaporated to give 160 mg brown oily residue. Purification by FCC (hexane/EtOAc, gradient). 6-Chloro-8-(1-methyl-1H-indol-6-yl)quinoxaline-2-carbonitrile (26.00 mg; 0.08 mmol; 15.2%; 97% by UPLC) is obtained as orange solid.

### Example 201

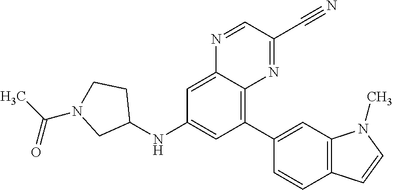 [SMILES: CC(=O)N1CCC(Nc2cc(-c3ccc4ccn(C)c4c3)c3nc(C#N)cnc3c2)C1]

The product is prepared according to the modified General Procedure 2, described in Example 1 with 6-Chloro-8-(1-methyl-1H-indol-6-yl)quinoxaline-2-carbonitrile (Intermediate 177) (26.00 mg; 0.08 mmol; 1 eq), 1-[3-aminopyrrolidin-1-yl]ethan-1-one (12.7 mg; 0.09 mmol; 1.2 eq.), NaOtBu (9.1 mg; 0.09 mmol; 1.2 eq.), t-BuBrettPhos (4.25 mg; 0.01 mmol; 0.10 eq.), t-BuBrettPhos Pd G3 (3.4 mg; 0.01 mmol; 0.05 eq.) and [1,4]-dioxane (1.0 ml). Reaction is carried out in sealed tube at 90° C. for 18 h. Purification by preparative HPLC (ACN/0.1% aquas ammonia). 6-[(1-Acetylpyrrolidin-3-yl)amino]-8-(1-methyl-1H-indol-6-yl)quinoxaline-2-carboxamide (3.5 mg; 0.01 mmol; yield 9.6%; 88.8% by HPLC) is obtained as white solid.

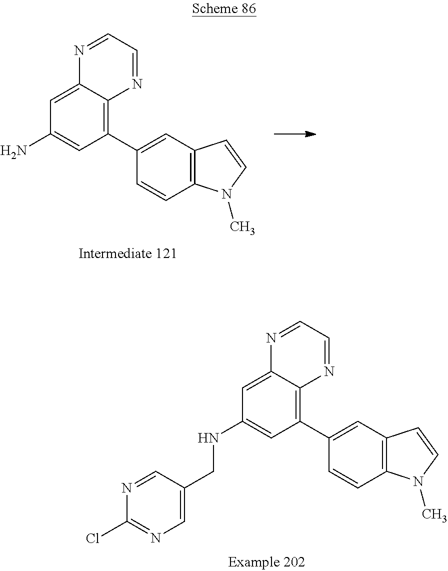 [SMILES: Cn1ccc2cc(-c3cc(N)cc4nccnc34)ccc21 ; Cn1ccc2cc(-c3cc(NCc4cnc(Cl)nc4)cc4nccnc34)ccc21]

### Example 202

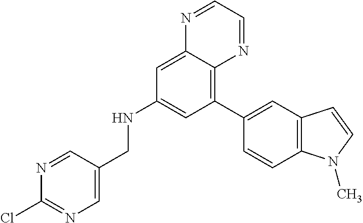 [SMILES: Cn1ccc2cc(-c3cc(NCc4cnc(Cl)nc4)cc4nccnc34)ccc21]

The product is prepared according to General Procedure 23, described in Example 63 with 8-(1-methyl-1H-indol-5-yl)quinoxalin-6-amine (Intermediate 121) (100.00 mg; 0.36 mmol; 1.00 eq.), 2-chloropyrimidine-5-carbaldehyde (69.33 mg; 0.46 mmol; 1.30 eq.), Hantzsch ester (118.46 mg; 0.47 mmol; 1.32 eq.), TMCS (18.41 μl; 0.14 mmol; 0.40 eq.) and DCE (8.00 ml). Reaction is carried out overnight at rt. RM is evaporated and the residue is purified by FCC (DCM/MeOH; gradient) (twice) to give N-[(2-chloropyrimidin-5-yl)methyl]-8-(1-methyl-1H-indol-5-yl)quinoxalin-6-amine (30.20 mg; yield 19.8%; 93.5% by HPLC) as a yellow powder.

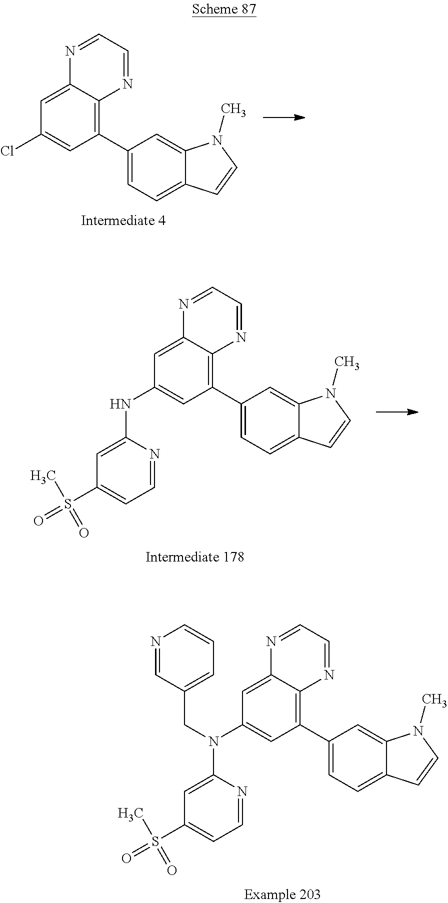 [SMILES: Cn1ccc2ccc(-c3cc(Cl)cc4nccnc34)cc21 ; Cn1ccc2ccc(-c3cc(Nc4cc(S(C)(=O)=O)ccn4)cc4nccnc34)cc21 ; Cn1ccc2ccc(-c3cc(N(Cc4cccnc4)c4cc(S(C)(=O)=O)ccn4)cc4nccnc34)cc21]

### Intermediate 178

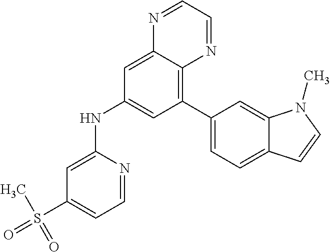 [SMILES: Cn1ccc2ccc(-c3cc(Nc4cc(S(C)(=O)=O)ccn4)cc4nccnc34)cc21]

The title compound is prepared according to General Procedure 2 described in Example 1, with 7-chloro-5-(1-methyl-1H-indol-6-yl)quinoxaline (Intermediate 4) (50 mg; 0.17 mmol; 1 eq.), 4-methanesulfonylpyridin-3-ylamine hydrochloride (43 mg; 0.20 mmol; 1.2 eq.), tBuONa (49 mg; 0.51 mmol; 3 eq.), BINAP (11 mg; 0.02 mmol; 0.1 eq.), Pd₂(dba)₃ (8 mg; 0.01 mmol; 0.05 eq.) in toluene (2 mL). Purification by FCC (EtOAc/hexane; gradient, next EtOAc/MeOH; gradient) provided N-(4-methanesulfonylpyridin-3-yl)-8-(1-methyl-H-indol-6-yl)quinoxalin-6-amine (50 mg; yield 68%; yellow powder; 99.3% by HPLC).

### Example 203—General Procedure 57

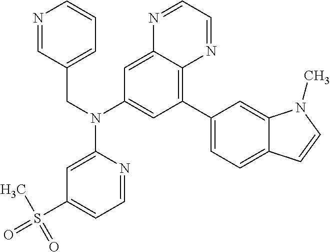 [SMILES: Cn1ccc2ccc(-c3cc(N(Cc4cccnc4)c4cc(S(C)(=O)=O)ccn4)cc4nccnc34)cc21]

A solution of N-(2-methanesulfonylpyridin-4-yl)-8-(1-methyl-1H-indol-6-yl)quinoxalin-6-amine (Intermediate 178) (70 mg; 0.12 mmol; 1 eq.) in anhydrous THF (2 mL) is cooled in an ice bath and NaH (60% in mineral oil, 15 mg; 0.37 mmol; 3 eq.) is added. The reaction mixture is stirred for 15 min., 3-bromomethyl-pyridine hydrobromide (37 mg; 0.15 mmol; 1.20 eq.) is added and stirring is continued overnight at room temperature. The reaction mixture is then poured onto ice and extracted twice with EtOAc. The combined organic layers are washed with brine, dried over Na₂SO₄ and filtered through a pad of Celite®. The filtrate is concentrated in vacuo and the crude product is purified by FCC (MeOH/EtOAc; gradient) to give N-(4-methanesulfonylpyridin-2-yl)-8-(1-methyl-1H-indol-6-yl)-N-[(pyridin-3-yl)methyl]quinoxalin-6-amine (29 mg; yield 44%; 97% by HPLC) is obtained as a yellow powder.

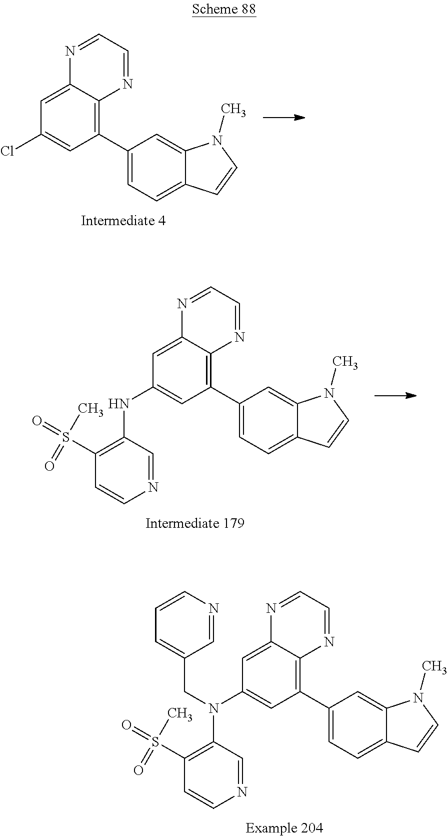 [SMILES: Cn1ccc2ccc(-c3cc(Cl)cc4nccnc34)cc21 ; Cn1ccc2ccc(-c3cc(Nc4cnccc4S(C)(=O)=O)cc4nccnc34)cc21 ; Cn1ccc2ccc(-c3cc(N(Cc4cccnc4)c4cnccc4S(C)(=O)=O)cc4nccnc34)cc21]

### Intermediate 179

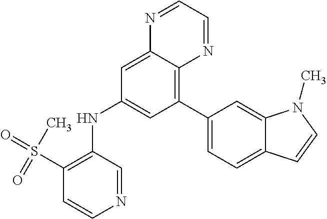 [SMILES: Cn1ccc2ccc(-c3cc(Nc4cnccc4S(C)(=O)=O)cc4nccnc34)cc21]

The title compound is prepared according to General Procedure 2 described in Example 1, with 7-chloro-5-(1-methyl-1H-indol-6-yl)quinoxaline (Intermediate 4) (50 mg; 0.17 mmol; 1 eq.), 4-methanesulfonylpyridin-3-ylamine hydrochloride (43 mg; 0.20 mmol; 1.2 eq.), tBuONa (49 mg; 0.51 mmol; 3 eq.), BINAP (11 mg; 0.02 mmol; 0.1 eq.), Pd₂(dba)₃ (8 mg; 0.01 mmol; 0.05 eq.) in toluene (2 mL). Purification by FCC (hexane/EtOAc; gradient, next EtOAc/MeOH; gradient) provided N-(4-methanesulfonylpyridin-3-yl)-8-(1-methyl-1H-indol-6-yl)quinoxalin-6-amine (50 mg; yield 68%; yellow powder; 99% by HPLC).

### Example 204

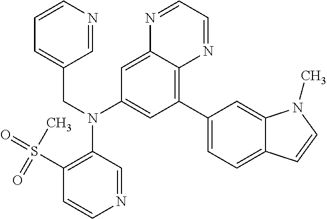 [SMILES: Cn1ccc2ccc(-c3cc(N(Cc4cccnc4)c4cnccc4S(C)(=O)=O)cc4nccnc34)cc21]

The title compound is prepared according to General Procedure 57 described in Example 203, using N-(4-methanesulfonylpyridin-3-yl)-8-(1-methyl-1H-indol-6-yl)quinoxalin-6-amine (Intermediate 179) (70 mg; 0.15 mmol; 1 eq.), NaH (60% in mineral oil, 37 mg; 0.92 mmol; 6 eq.), 3-bromomethylpyridine hydrobromide (124 mg; 0.49 mmol; 3.20 eq.) in anhydrous THF (6 mL). Conditions: overnight at room temperature. Purification by FCC (DCM/MeOH; gradient) provided N-(4-methanesulfonylpyridin-3-yl)-8-(1-methyl-1H-indol-6-yl)-N-[(pyridin-3-yl)methyl]quinoxalin-6-amine (15 mg; yield 18%; yellow powder; 95% by HPLC).

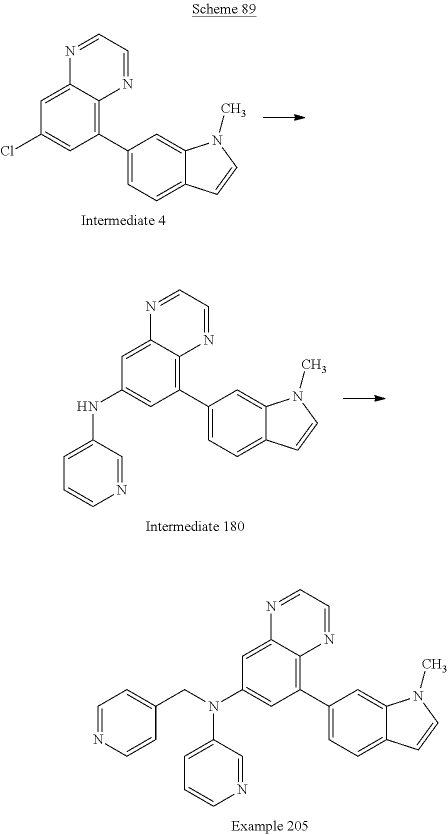 [SMILES: Cn1ccc2ccc(-c3cc(Cl)cc4nccnc34)cc21 ; Cn1ccc2ccc(-c3cc(Nc4cccnc4)cc4nccnc34)cc21 ; Cn1ccc2ccc(-c3cc(N(Cc4ccncc4)c4cccnc4)cc4nccnc34)cc21]

### Intermediate 180

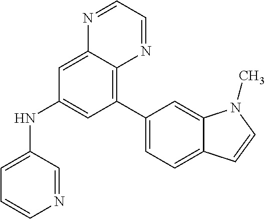 [SMILES: Cn1ccc2ccc(-c3cc(Nc4cccnc4)cc4nccnc34)cc21]

The title compound is prepared according to General Procedure 2 described in Example 1, using 7-chloro-5-(1-methyl-1H-indol-6-yl)quinoxaline (Intermediate 4) (100 mg; 0.31 mmol; 1 eq.), pyridin-2-ylamine (37 mg; 0.37 mmol; 1.20 eq.), Cs₂CO₃ (255 mg; 0.77 mmol; 2.50 eq.), BINAP (20 mg; 0.03 mmol; 0.10 eq.), Pd(OAc)₂ (7 mg; 0.03 mmol; 0.10 eq.) and anhydrous 1,4-dioxane (1 mL). Conditions: 150. C for 3 hours. Purification by FCC (hexane/EtOAc; gradient) provided 8-(1-methyl-1H-indol-6-yl)-N-(pyridin-2-yl)quinoxalin-6-amine (96 mg; yield 86%; yellow powder; 97% by HPLC).

### Example 205

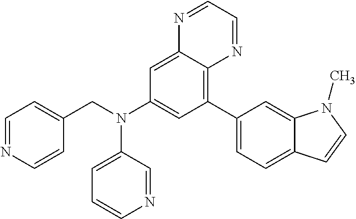 [SMILES: Cn1ccc2ccc(-c3cc(N(Cc4ccncc4)c4cccnc4)cc4nccnc34)cc21]

The title compound is prepared according to General Procedure 57 described in Example 203, using 8-(1-methyl-1H-indol-6-yl)-N-(pyridin-2-yl)quinoxalin-6-amine (Intermediate 180) (60 mg; 0.17 mmol; 1 eq.), NaH (60% in mineral oil, 20 mg; 0.50 mmol; 3 eq.), 3-bromomethylpyridine hydrobromide (51 mg; 0.20 mmol; 1.20 eq.) in anhydrous anhydrous THF (2 mL). Conditions: overnight at room temperature. Purification by FCC (hexane/EtOAc; gradient, next EtOAc/MeOH; gradient) provided 8-(1-methyl-1H-indol-6-yl)-N-(pyridin-2-yl)-N-[(pyridin-3-yl)methyl]quinoxalin-6-amine (60 mg; yield 79%; yellow powder; 98% by HPLC).

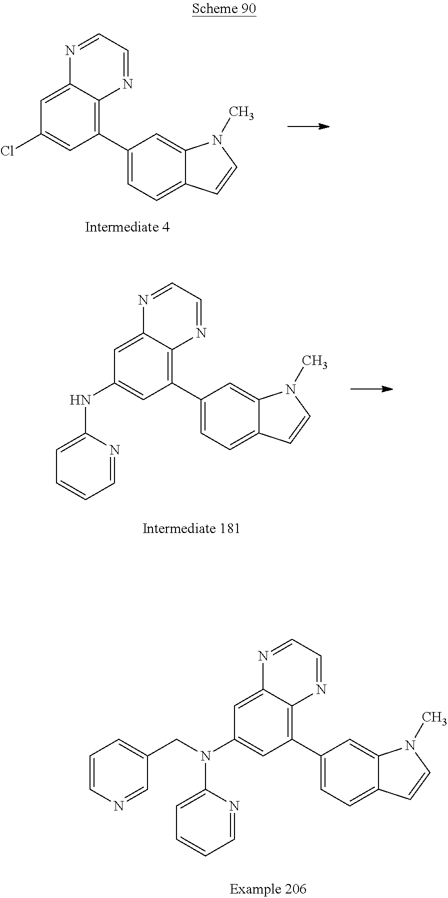 [SMILES: Cn1ccc2ccc(-c3cc(Cl)cc4nccnc34)cc21 ; Cn1ccc2ccc(-c3cc(Nc4ccccn4)cc4nccnc34)cc21 ; Cn1ccc2ccc(-c3cc(N(Cc4cccnc4)c4ccccn4)cc4nccnc34)cc21]

### Intermediate 181

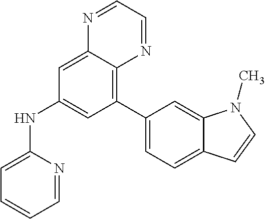 [SMILES: Cn1ccc2ccc(-c3cc(Nc4ccccn4)cc4nccnc34)cc21]

The title compound is prepared according to General Procedure 2 described in Example 1, using 7-chloro-5-(1-methyl-1H-indol-6-yl)quinoxaline (Intermediate 4) (100 mg; 0.31 mmol; 1 eq.), pyridin-2-ylamine (37 mg; 0.37 mmol; 1.20 eq.), Cs₂CO₃ (255 mg; 0.77 mmol; 2.50 eq.), BINAP (20 mg; 0.03 mmol; 0.10 eq.), Pd(OAc)₂ (7 mg; 0.03 mmol; 0.10 eq.) and anhydrous 1,4-dioxane (1 mL). Conditions: 150° C. for 3 hours. Purification by FCC (hexane/EtOAc; gradient) provided 8-(1-methyl-1H-indol-6-yl)-N-(pyridin-2-yl)quinoxalin-6-amine (96 mg; yield 86%; yellow powder; 97% by HPLC).

### Example 206

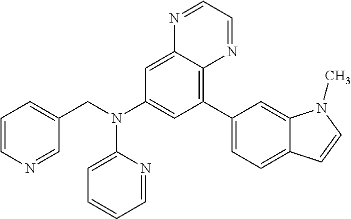 [SMILES: Cn1ccc2ccc(-c3cc(N(Cc4cccnc4)c4ccccn4)cc4nccnc34)cc21]

The title compound is prepared according to General Procedure 57 described in Example 203, using 8-(1-methyl-1H-indol-6-yl)-N-(pyridin-2-yl)quinoxalin-6-amine (Intermediate 181) (60 mg; 0.17 mmol; 1 eq.), NaH (60% in mineral oil, 20 mg; 0.50 mmol; 3 eq.), 3-bromomethylpyridine hydrobromide (51 mg; 0.20 mmol; 1.20 eq.) in anhydrous THF (2 mL). Conditions: overnight at room temperature. Purification by FCC (hexane/EtOAc; gradient, next EtOAc/MeOH; gradient) provided 8-(1-methyl-1H-indol-6-yl)-N-(pyridin-2-yl)-N—[(pyridin-3-yl)methyl]quinoxalin-6-amine (60 mg; yield 79%; yellow powder; 98% by HPLC).

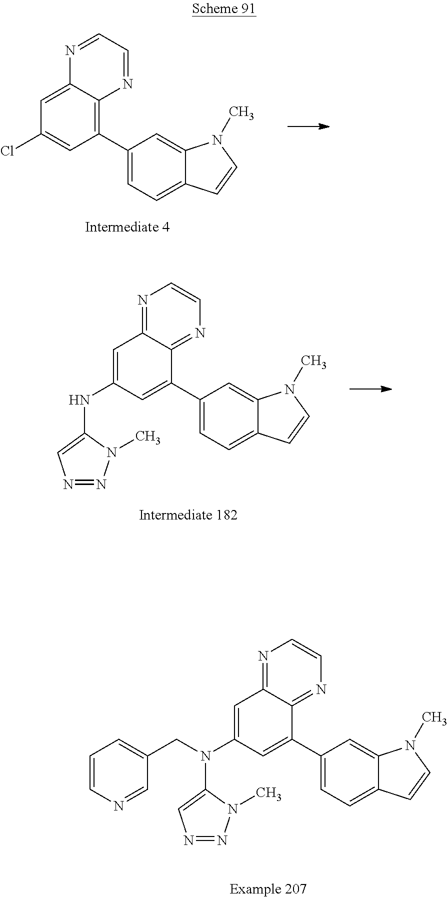 [SMILES: Cn1ccc2ccc(-c3cc(Cl)cc4nccnc34)cc21 ; Cn1nncc1Nc1cc(-c2ccc3ccn(C)c3c2)c2nccnc2c1 ; Cn1nncc1N(Cc1cccnc1)c1cc(-c2ccc3ccn(C)c3c2)c2nccnc2c1]

### Intermediate 182

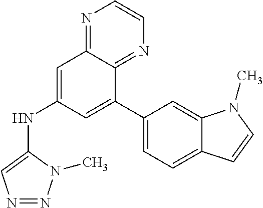 [SMILES: Cn1nncc1Nc1cc(-c2ccc3ccn(C)c3c2)c2nccnc2c1]

The title compound is prepared according to General Procedure 2 described in Example 1, using 7-chloro-5-(1-methyl-1H-indol-6-yl)quinoxaline (Intermediate 4) (103 mg; 0.34 mmol; 1.10 eq.), 3-methyl-3H-[1,2,3]triazol-4-ylamine (30 mg; 0.31 mmol; 1 eq.), Cs₂CO₃ (252 mg; 0.76 mmol; 2.50 eq.), BINAP (20 mg; 0.03 mmol; 0.10 eq.) and Pd(OAc)₂ (7 mg; 0.03 mmol; 0.10 eq.) in 1,4-dioxane anhydrous (2 mL). Conditions: 120° C. overnight. Purification by FCC (hexane/EtOAc; gradient, next EtOAc/MeOH; gradient) affords N-(1-methyl-1H-1,2,3-triazol-5-yl)-8-(1-methyl-1H-indol-6-yl)quinoxalin-6-amine (24 mg, yield 21%; yellow powder; 93% by HPLC).

### Example 207

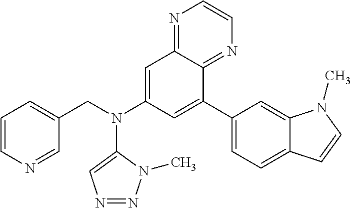 [SMILES: Cn1nncc1N(Cc1cccnc1)c1cc(-c2ccc3ccn(C)c3c2)c2nccnc2c1]

The title compound is prepared according to General Procedure 57 described in Example 203, using N-(1-methyl-1H-1,2,3-triazol-5-yl)-8-(1-methyl-1H-indol-6-yl)quinoxalin-6-amine (Intermediate 182) (27 mg; 0.05 mmol; 1 eq.), NaH (60% in mineral oil, 5 mg; 0.13 mmol; 2.50 eq.) in dry tetrahydrofuran (3 mL), and a suspension of 3-chloromethylpyridine hydrochloride (10 mg; 0.06 mmol; 1.20 eq.) and triethylamine (8 μl; 0.06 mmol; 1.20 eq.) in dry DMF (1 mL). Conditions: overnight at room temperature. Purification by FCC (hexane/EtOAc; gradient, next EtOAC/MeOH; gradient) provided N-(1-methyl-1H-1,2,3-triazol-5-yl)-8-(1-methyl-1H-indol-6-yl)-N-[(pyridin-3-yl)methyl]quinoxalin-6-amine (11 mg; yield 46%; yellow powder; 97% by HPLC).

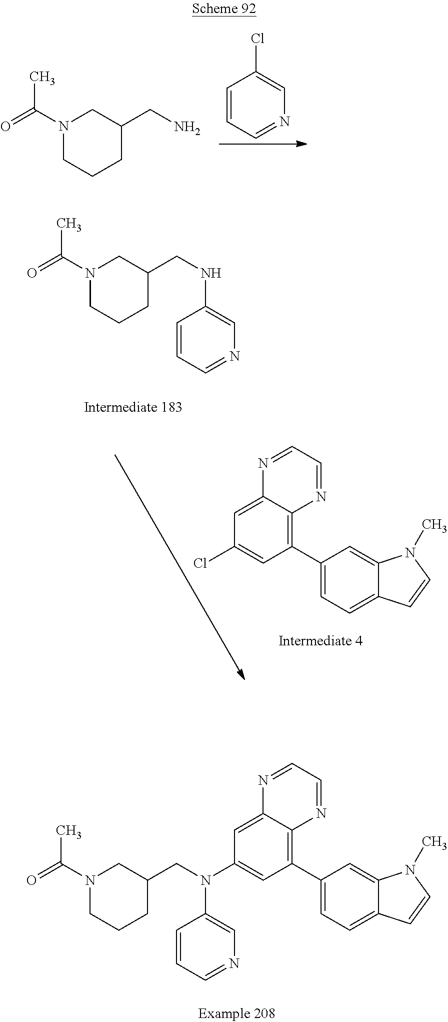 [SMILES: CC(=O)N1CCCC(CN)C1 ; Clc1cccnc1 ; CC(=O)N1CCCC(CNc2cccnc2)C1 ; Cn1ccc2ccc(-c3cc(Cl)cc4nccnc34)cc21 ; CC(=O)N1CCCC(CN(c2cccnc2)c2cc(-c3ccc4ccn(C)c4c3)c3nccnc3c2)C1]

### Intermediate 183

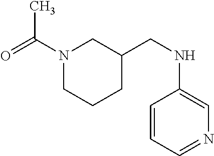 [SMILES: CC(=O)N1CCCC(CNc2cccnc2)C1]

The title compound is prepared according to General Procedure 2 described in Example 1, using 1-(3-aminomethylpiperidin-1-yl)-ethanone (200 mg; 1.22 mmol; 1 eq.), 3-chloropyridine (0.14 mL; 1.46 mmol; 1.20 eq.), Cs₂CO₃ (1 g; 3.04 mmol; 2.50 eq.), BINAP (77 mg; 0.12 mmol; 0.10 eq.) and Pd(OAc)₂ (28 mg; 0.12 mmol; 0.10 eq.) in anhydrous 1,4-dioxane (3 mL). Conditions: 120° C. overnight. Purification by FCC (DCM/MeOH; gradient) provided 1-(3-{[(pyridin-3-yl)amino]methyl}piperidin-1-yl)ethan-1-one (125 mg; yield 44%; orange oil; 99% by UPLC).

### Example 208 General Procedure 58

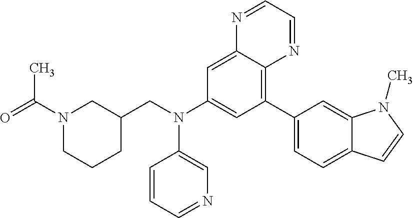 [SMILES: CC(=O)N1CCCC(CN(c2cccnc2)c2cc(-c3ccc4ccn(C)c4c3)c3nccnc3c2)C1]

A microwave vial is charged with 7-chloro-5-(1-methyl-1H-indol-6-yl)quinoxaline (Intermediate 4) (135 mg; 0.44 mmol; 1 eq.), 1-(3-{[(pyridin-3-yl)-amino]methyl}piperidin-1-yl)ethan-1-one (Intermediate 183) (125 mg; 0.53 mmol; 1.20 eq.), tBuONa (51 mg; 0.53 mmol; 1.20 eq.), RuPhos (22 mg; 0.04 mmol; 0.10 eq.) and RuPhos Pd G3 (39 mg; 0.04 mmol; 0.10 eq.). The tube is closed with a rubber septum, evacuated and backfilled with argon three times. Anhydrous THF (3 mL) is added via syringe before sealing the vial, and the reaction mixture is stirred at 85° C. overnight. It is then cooled to room temperature, diluted with EtOAc and filtered through a pad of Celite®. The solvents are evaporated and the crude product is purified by FCC (EtOAc/MeOH; gradient) to afford 1-[3-({[8-(1-methyl-1H-indol-6-yl)quinoxalin-6-yl](pyridin-3-yl)amino}methyl)piperidin-1-yl]ethan-1-one (113 mg; yield 51%; yellow powder; 98% by HPLC).

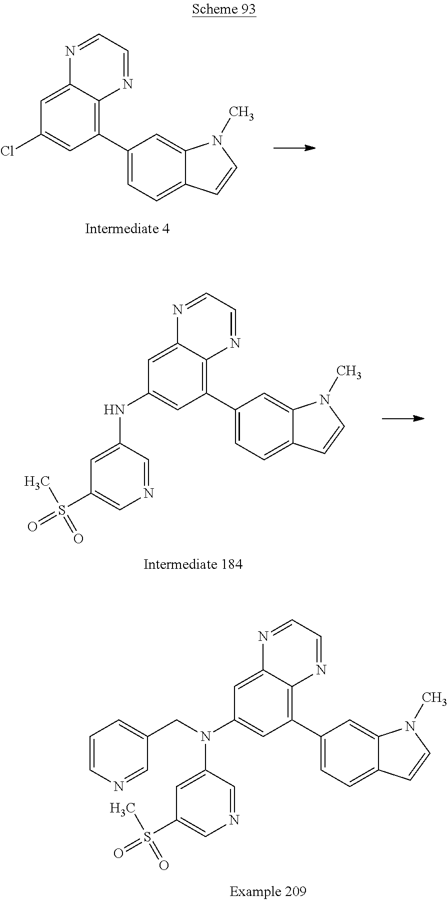 [SMILES: Cn1ccc2ccc(-c3cc(Cl)cc4nccnc34)cc21 ; Cn1ccc2ccc(-c3cc(Nc4cncc(S(C)(=O)=O)c4)cc4nccnc34)cc21 ; Cn1ccc2ccc(-c3cc(N(Cc4cccnc4)c4cncc(S(C)(=O)=O)c4)cc4nccnc34)cc21]

### Intermediate 184

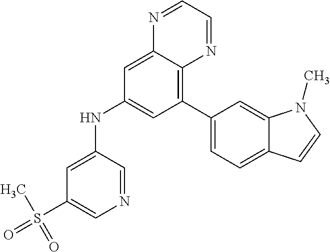 [SMILES: Cn1ccc2ccc(-c3cc(Nc4cncc(S(C)(=O)=O)c4)cc4nccnc34)cc21]

The title compound is prepared according to General Procedure 2 described in Example 1, using 3-bromo-5-methanesulfonylpyridine (50 mg; 0.20 mmol; 1 eq.), 7-chloro-5-(1-methyl-1H-indol-6-yl)quinoxaline (Intermediate 4) (59 mg; 0.20 mmol; 1 eq.), Cs₂CO₃ (166 mg; 0.50 mmol; 2.50 eq.), BINAP (13 mg; 0.02 mmol; 0.10 eq.), Pd(OAc)₂ (5 mg; 0.02 mmol; 0.10 eq.) in anhydrous 1,4-dioxane (2 mL). Conditions: 120° C. for 16 h. Purification by FCC (hexane/EtOAc; gradient, next EtOAc/MeOH; gradient) provided N-(5-methanesulfonylpyridin-3-yl)-8-(1-methyl-1H-indol-6-yl)quinoxalin-6-amine (72 mg; yield 77%; yellow powder; 93% by HPLC).

### Example 209

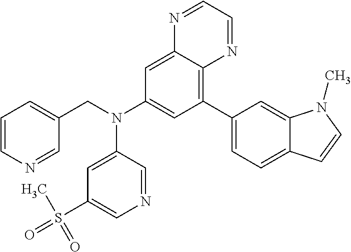 [SMILES: Cn1ccc2ccc(-c3cc(N(Cc4cccnc4)c4cncc(S(C)(=O)=O)c4)cc4nccnc34)cc21]

The title compound is prepared according to General Procedure 57 described in Example 203, using N-(5-methanesulfonylpyridin-3-yl)-8-(1-methyl-1H-indol-6-yl)quinoxalin-6-amine (Intermediate 184) (51 mg; 0.12 mmol; 1 eq.), NaH (60% in mineral oil, 14 mg; 0.35 mmol; 3 eq.), bromomethylpyridine hydrobromide (37 mg; 0.14 mmol; 1.20 eq.) in anhydrous THF (2 mL). Conditions: overnight at room temperature. Purification by FCC (DCM/MeOH; gradient) yields N-(5-methanesulfonylpyridin-3-yl)-8-(1-methyl-1H-indol-6-yl)-N—[(pyridin-3-yl)methyl]quinoxalin-6-amine (41 mg; yield 64%; yellow powder; 95% by HPLC).

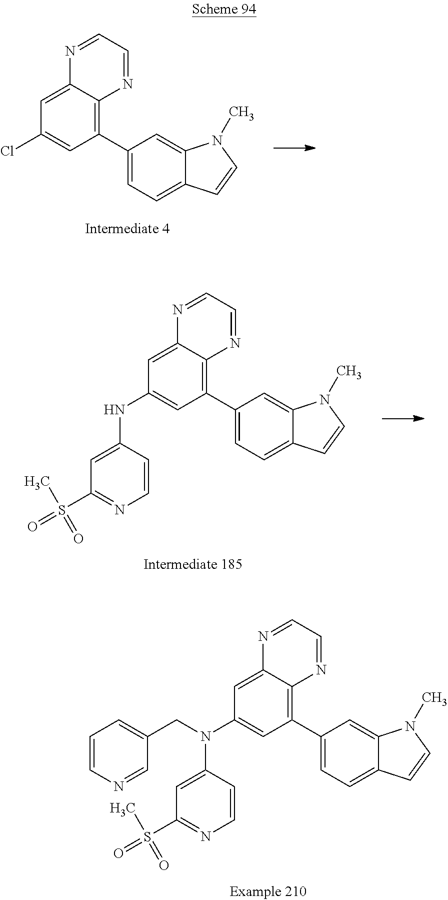 [SMILES: Cn1ccc2ccc(-c3cc(Cl)cc4nccnc34)cc21 ; Cn1ccc2ccc(-c3cc(Nc4ccnc(S(C)(=O)=O)c4)cc4nccnc34)cc21 ; Cn1ccc2ccc(-c3cc(N(Cc4cccnc4)c4ccnc(S(C)(=O)=O)c4)cc4nccnc34)cc21]

### Intermediate 185

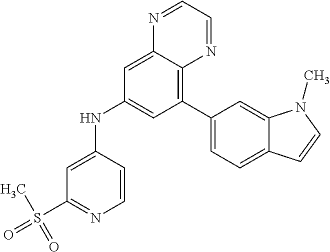 [SMILES: Cn1ccc2ccc(-c3cc(Nc4ccnc(S(C)(=O)=O)c4)cc4nccnc34)cc21]

The title compound is prepared according to General Procedure 2 described in Example 1, using 7-chloro-5-(1-methyl-1H-indol-6-yl)quinoxaline (Intermediate 4) (80 mg; 0.27 mmol; 1 eq.), 2-methanesulfonylpyridin-4-ylamine hydrochloride (76 mg; 0.35 mmol; 1.30 eq.), Cs₂CO₃ (306 mg; 0.93 mmol; 3.50 eq.), BINAP (26 mg; 0.04 mmol; 0.15 eq.) and Pd(OAc)₂ (9 mg; 0.04 mmol; 0.15 eq.) in anhydrous 1,4-dioxane (4 mL). Conditions: 125° C., 16 hours. Purification by FCC (hexane/EtOAc; gradient) provided N-(2-methanesulfonylpyridin-4-yl)-8-(1-methyl-1H-indol-6-yl)quinoxalin-6-amine (97 mg; yield 83%; yellow powder; 97% by HPLC).

### Example 210

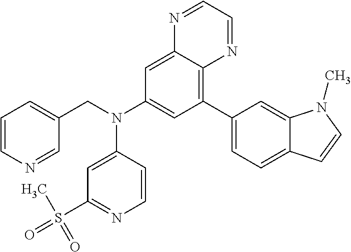 [SMILES: Cn1ccc2ccc(-c3cc(N(Cc4cccnc4)c4ccnc(S(C)(=O)=O)c4)cc4nccnc34)cc21]

The title compound is prepared according to General Procedure 97 described in Example 203, using N-(2-methanesulfonylpyridin-4-yl)-8-(1-methyl-1H-indol-6-yl)quinoxalin-6-amine (Intermediate 185) (70 mg; 0.16 mmol; 1 eq.), NaH (60% in mineral oil, 19 mg; 0.48 mmol; 3 eq.) and 3-bromomethylpyridine hydrobromide (51 mg; 0.19 mmol; 1.20 eq.) in anhydrous THF (2 mL). Conditions: room temperature overnight. Purification by FCC (DCM/MeOH; gradient) provided N-(2-methanesulfonylpyridin-4-yl)-8-(1-methyl-1H-indol-6-yl)-N-[(pyridin-3-yl)methyl]quinoxalin-6-amine (61 mg; yield 70%; yellow powder; 96% by HPLC).

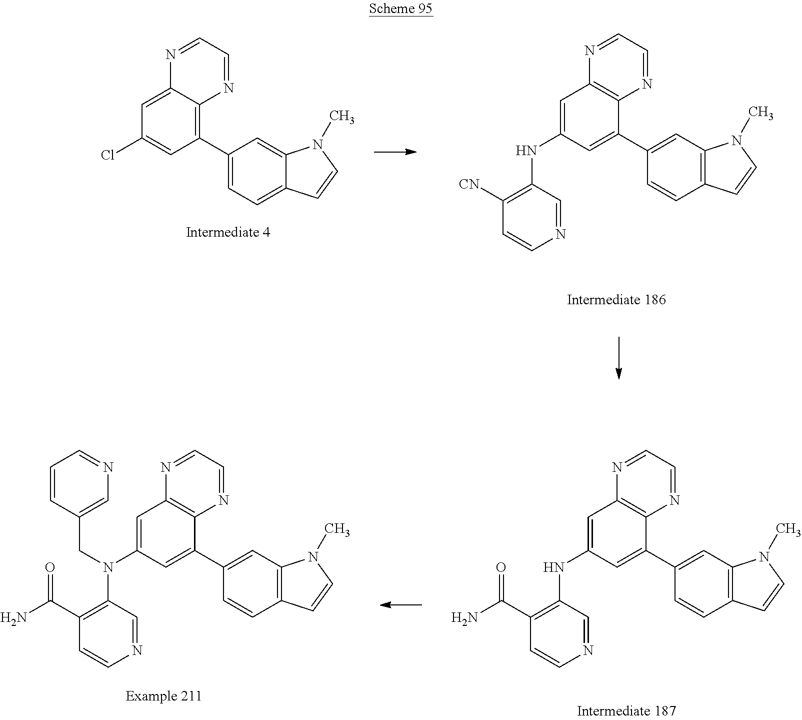

### Intermediate 186

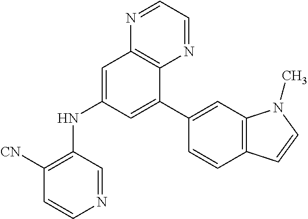

The title compound is prepared according to General Procedure 5 described in Example 30 with 7-chloro-5-(1-methyl-1H-indol-6-yl)quinoxaline (Intermediate 4) (60 mg; 0.20 mmol; 1 eq.), 3-amino-isonicotinonitrile (29 mg; 0.25 mmol; 1.2 eq.), K₂CO₃ (56 mg; 0.41 mmol; 2 eq.), BippyPhos (21 mg; 0.04 mmol; 0.2 eq.), (Pd(cinnamyl)Cl)₂ (4 mg; 0.01 mmol; 0.04 eq.) in 1,4-dioxane (3 mL). Conditions: 120° C. for 12 h. Purification by FCC (DCM/MeOH; gradient, column neutralized with 1% Et3N in DCM and washed with DCM beforehand) provided 3-{[8-(1-methyl-1H-indol-6-yl)quinoxalin-6-yl]amino}pyridine-4-carbonitrile (35 mg; yield 45%; yellow powder; 97% by HPLC).

### Intermediate 187

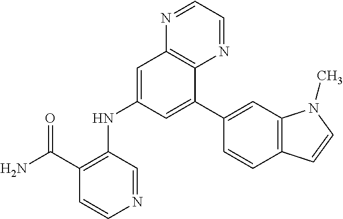 [SMILES: Cn1ccc2ccc(-c3cc(Nc4cnccc4C(N)=O)cc4nccnc34)cc21]

The title compound is prepared according to General Procedure 7 described in Example 35 with 3-{[8-(1-methyl-1H-indol-6-yl)quinoxalin-6-yl]amino}-pyridine-4-carbonitrile (Intermediate 186) (15 mg; 0.04 mmol; 1 eq.), KOH (7 mg; 0.12 mmol; 3 eq.) and t-BuOH (2 mL). The reaction mixture is stirred at 60° C. under argon for 5 h. 3-{[8-(1-methyl-1H-indol-6-yl)quinoxalin-6-yl]amino}pyridine-4-carboxamide (8 mg; yield 48%; 92% by HPLC) is obtained as a yellow powder.

### Example 211

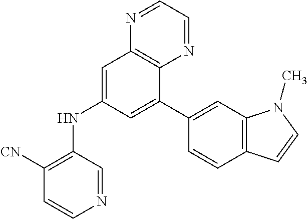

The title compound is prepared according to General Procedure 57 described in Example 203, using 3-{[8-(1-methyl-1H-indol-6-yl)quinoxalin-6-yl]amino}-N-(pyrimidin-5-yl)pyridine-4-carboxamide (Intermediate 187) (20 mg; 0.05 mmol; 1 eq.), NaH (60% in mineral oil, 6 mg; 0.14 mmol; 3 eq.), 3-bromomethylpyridine hydrobromide (15 mg; 0.06 mmol; 1.20 eq.) in anhydrous THF. Conditions: overnight at room temperature. Purification by FCC (hexane/EtOAc; gradient) provided 3-{[8-(1-methyl-1H-indol-6-yl)quinoxalin-6-yl][(pyridin-3-yl)methyl]amino}pyridine-4-carboxamide (6 mg; yield 24%; yellow powder; 92% by HPLC).

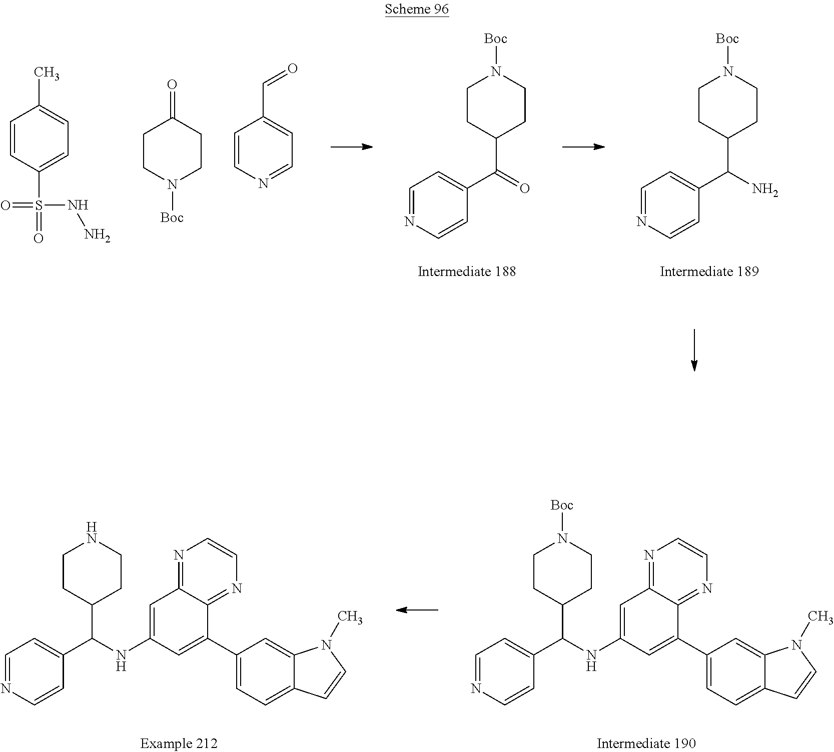

### Intermediate 188

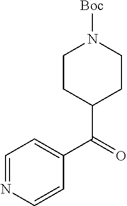 [SMILES: CC(C)(C)OC(=O)N1CCC(C(=O)c2ccncc2)CC1]

The product is prepared according to General Procedure 10, described for Intermediate 10, with 4-oxo-piperidine-1-carboxylic acid tert-butyl ester (500.00 mg; 2.51 mmol; 1.00 eq.), 4-methylbenzenesulfonohydrazide (467.34 mg; 2.51 mmol; 1.00 eq.), CS₂CO₃ (613.22 mg; 1.88 mmol; 0.75 eq.), pyridine-4-carbaldehyde (0.24 ml; 2.51 mmol; 1.00 eq.), methanol (5 mL) and 1,4-dioxane (5 mL). Purification by FCC (column is deactivated with 1% Et₃N/DCM and DCM; DCM/MeOH; gradient). 4-(Pyridine-4-carbonyl)-piperidine-1-carboxylic acid tert-butyl ester (200.00 mg; yield 21%; 77% by UPLC) is obtained as a colorless oil.

### Intermediate 189

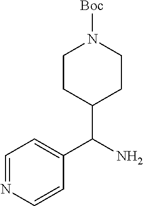 [SMILES: CC(C)(C)OC(=O)N1CCC(C(N)c2ccncc2)CC1]

The product is prepared according to General Procedure 14, described for Intermediate 12, with 4-(pyridine-4-carbonyl)-piperidine-1-carboxylic acid tert-butyl ester (Intermediate 188) (200.00 mg; 0.53 mmol; 1.00 eq.), TTIP (0.31 ml; 1.06 mmol; 2.00 eq.), NaBH₄ (80.26 mg; 2.12 mmol; 4.00 eq.) and 7M NH₃ in MeOH (2.50 mL). Purification by FCC (EtOAc/MeOH; gradient). 4-Amino-pyridin-4-yl-methyl)-piperidine-1-carboxylic acid tert-butyl ester (81.00 mg; yield 52.4%; 100% by UPLC) is obtained as a colorless oil.

### Intermediate 190

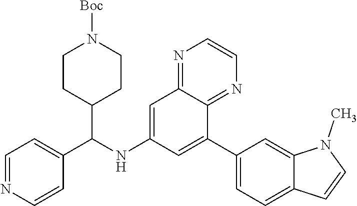 [SMILES: Cn1ccc2ccc(-c3cc(NC(c4ccncc4)C4CCN(C(=O)OC(C)(C)C)CC4)cc4nccnc34)cc21]

The product is prepared according to General Procedure 2, described in Example 1 with 7-chloro-5-(1-methyl-1H-indol-6-yl)-quinoxaline (Intermediate 4) (55.00 mg; 0.18 mmol; 1.00 eq.), 4-(amino-pyridin-4-yl-methyl)-piperidine-1-carboxylic acid tert-butyl ester (Intermediate 189) (80.20 mg; 0.28 mmol; 1.50 eq.), NaOtBu (61.72 mg; 0.64 mmol; 3.50 eq.), Pd₂(dba)₃, BINAP (22.85 mg; 0.04 mmol; 0.20 eq.) and toluene (5 mL). RM is stirred at 120° C. overnight. Purification by FCC (EtOAc/MeOH; gradient). 4-{[8-(1-Methyl-1H-indol-6-yl)-quinoxalin-6-ylamino]-pyridin-4-yl-methyl}-piperidine-1-carboxylic acid tert-butyl ester (75.00 mg; yield 74%; 99% by UPLC) is obtained as yellow amorphous powder.

### Example 212

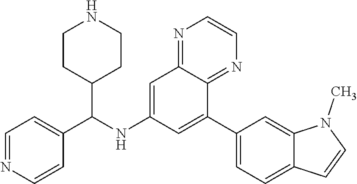 [SMILES: Cn1ccc2ccc(-c3cc(NC(c4ccncc4)C4CCNCC4)cc4nccnc34)cc21]

The product is prepared according to General Procedure 10, described in Example 44 with 4-{[8-(1-methyl-1H-indol-6-yl)-quinoxalin-6-ylamino]-pyridin-4-yl-methyl}-piperidine-1-carboxylic acid tert-butyl ester (Intermediate 190) (55.00 mg; 0.10 mmol; 1.00 eq.), TFA (1.00 ml; 13.07 mmol; 133.02 eq.) and DCM anhydrous (5.00 mL). Purification by HPLC. [8-(1-Methyl-1H-indol-6-yl)-quinoxalin-6-yl]-(piperidin-4-yl-pyridin-4-yl-methyl)-amine formic acid (5.30 mg; yield 10.5%; 92% by HPLC) is obtained as an orange amorphous powder.

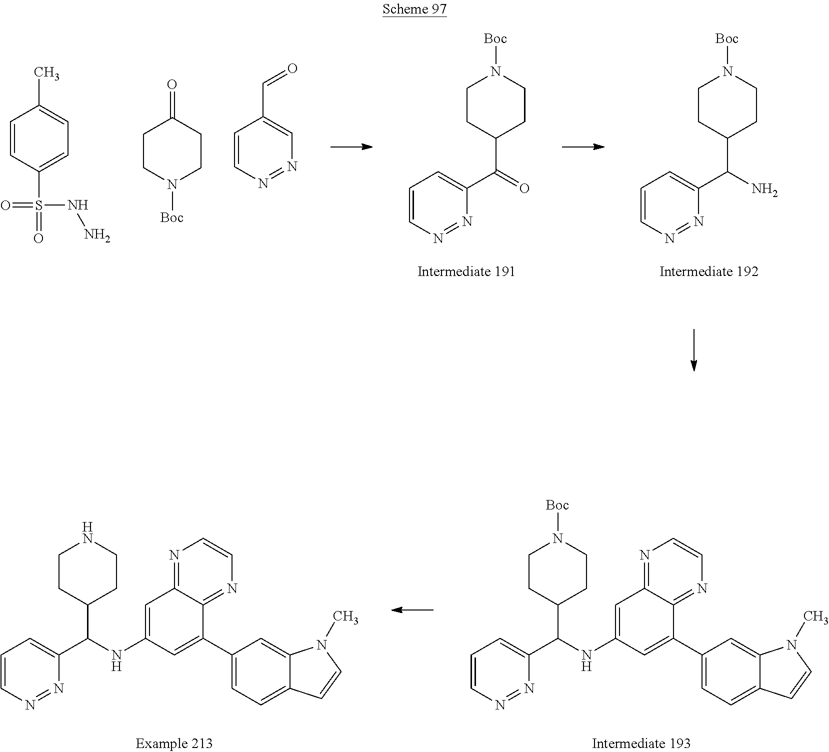

### Intermediate 191

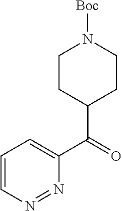 [SMILES: CC(C)(C)OC(=O)N1CCC(C(=O)c2cccnn2)CC1]

The product is prepared according to General Procedure 15, described for Intermediate 13, with 4-oxo-piperidine-1-carboxylic acid tert-butyl ester (450.00 mg; 2.26 mmol; 1.00 eq.), 4-methylbenzenesulfonohydrazide (420.61 mg; 2.26 mmol; 1.00 eq.), Cs₂O₃ (0.55 g; 1.69 mmol; 0.75 eq.), pyridazine-3-carbaldehyde (244.14 mg; 2.26 mmol; 1.00 eq.), MeOH (10 mL) and 1,4-dioxane (10 mL). Purification by FCC (column is deactivated with 1% Et₃N/DCM and DCM; hexane/EtOAc; gradient). 4-(Pyridazine-3-carbonyl)-piperidine-1-carboxylic acid tert-butyl ester (93.00 mg; yield 13.4%; 95% by UPLC) is obtained as a yellow solid.

### Intermediate 192

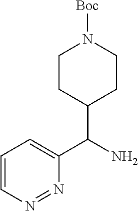 [SMILES: CC(C)(C)OC(=O)N1CCC(C(N)c2cccnn2)CC1]

The product is prepared according to General Procedure 14, described for Intermediate 12, with 4-(pyridazine-3-carbonyl)-piperidine-1-carboxylic acid tert-butyl ester (Intermediate 191) (92.00 mg; 0.30 mmol; 1.00 eq.), TTIP (0.18 ml; 0.60 mmol; 2.00 eq.), NaBH₄ (45.30 mg; 1.20 mmol; 4.00 eq.) and 7M NH₃ in MeOH (2.50 mL). Purification by FCC (EtOAc/MeOH; gradient). 4-(Aminopyridazin-3-yl-methyl)-piperidine-1-carboxylic acid tert-butyl ester (75.00 mg; yield 81.9%; 95% by UPLC) is obtained as a colorless oil.

### Intermediate 193

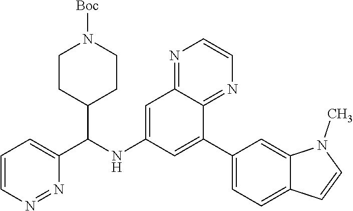 [SMILES: Cn1ccc2ccc(-c3cc(NC(c4cccnn4)C4CCN(C(=O)OC(C)(C)C)CC4)cc4nccnc34)cc21]

The product is prepared according to General Procedure 2, described in Example 1 with 7-chloro-5-(1-methyl-1H-indol-6-yl)-quinoxaline (Intermediate 4) (48.00 mg; 0.16 mmol; 1.00 eq.), 4-(Amino-pyridazin-3-yl-methyl)-piperidine-1-carboxylic acid tert-butyl ester (Intermediate 191) (74.96 mg; 0.25 mmol; 1.50 eq.), NaOtBu (54.96 mg; 0.57 mmol; 3.50 eq.), Pd₂(dba)₃ (16.91 mg; 0.02 mmol; 0.10 eq.), BINAP (20.35 mg; 0.03 mmol; 0.20 eq.) and toluene (5 mL). The reaction mixture was sealed and stirred at 120° C. overnight. Purification by FCC (DCM/MeOH; gradient). 4{[8-(1-Methyl-1H-indol-6-yl)-quinoxalin-6-ylamino]-pyridazin-3-yl-methyl}-piperidine-1-carboxylic acid tert-butyl ester (56.00 mg; yield 55.2%; 89% by UPLC) is obtained as a yellow oil.

### Example 213

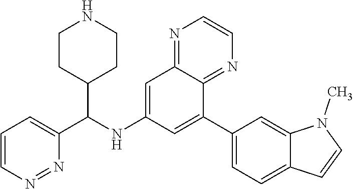 [SMILES: Cn1ccc2ccc(-c3cc(NC(c4cccnn4)C4CCNCC4)cc4nccnc34)cc21]

The product is prepared according to General Procedure 4, described in Example 25 with 4-{[8-(1-methyl-1H-indol-6-yl)-quinoxalin-6-ylamino]-pyridazin-3-yl-methyl}-piperidine-1-carboxylic acid tert-butyl ester (Intermediate 193) (56.00 mg; 0.09 mmol; 1.00 eq.) and 4N HCl in 1,4-dioxane (3.00 mL; 12.00 mmol; 132.94 eq.). Purification by HPLC. [8-(1-methyl-1H-indol-6-yl)-quinoxalin-6-yl]-(piperidin-4-yl-pyridazin-3-yl-methyl)-amine formic acid (1.60 mg; yield 3.6%; 100% by HPLC) is obtained as an orange powder.

### Example 214

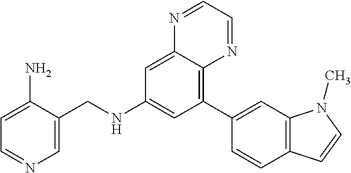 [SMILES: Cn1ccc2ccc(-c3cc(NCc4cnccc4N)cc4nccnc34)cc21]

The product is prepared according to General Procedure 5, described in Example 30 with 7-chloro-5-(1-methyl-1H-indol-6-yl)-quinoxaline (Intermediate 4, 75.00 mg; 0.26 mmol; 1.00 eq.), 3-aminomethylpyridin-4-ylamine (34.59 mg; 0.28 mmol; 1.10 eq.), NaOtBu (34.35 mg; 0.36 mmol; 1.40 eq.), [(Cinnamyl)PdCl]₂ (6.61 mg; 0.01 mmol; 0.05 eq.), BippyPhos (10.35 mg; 0.02 mmol; 0.08 eq.) and toluene anhydrous (5 mL). RM was stirred at 110° C. overnight in a sealed tube. Purification by FCC (hexane/EtOAc/MeOH; gradient). (4-Amino-pyridin-3-ylmethyl)-[8-(1-methyl-1H-indol-6-yl)-quinoxalin-6-yl]-amine (45.50 mg; yield 45.8%; 98% by HPLC) is obtained as a yellow solid.

### Example 215

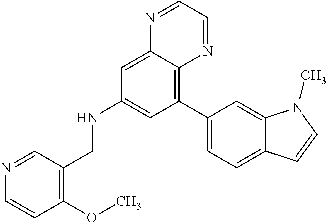 [SMILES: COc1ccncc1CNc1cc(-c2ccc3ccn(C)c3c2)c2nccnc2c1]

The product is prepared according to General Procedure 23, described in Example 63 with 4-methoxy-pyridine-3-carbaldehyde (0.04 ml; 0.26 mmol; 1.50 eq.), 8-(1-methyl-1H-indol-6-yl)-quinoxalin-6-ylamine (Intermediate 22, 50.00 mg; 0.18 mmol; 1.00 eq.), Hantzsch ester (66.90 mg; 0.26 mmol; 1.50 eq.), TMCS (0.01 ml; 0.05 mmol; 0.30 eq.) and DCM (5 mL). Purification by FFC (hexane/EtOAc/MeOH; gradient). (4-Methoxy-pyridin-3-ylmethyl)-[8-(1-methyl-1H-indol-6-yl)-quinoxalin-6-yl]-amine (56.90 mg; yield 81.1%; by HPLC) is obtained as a bright yellow solid.

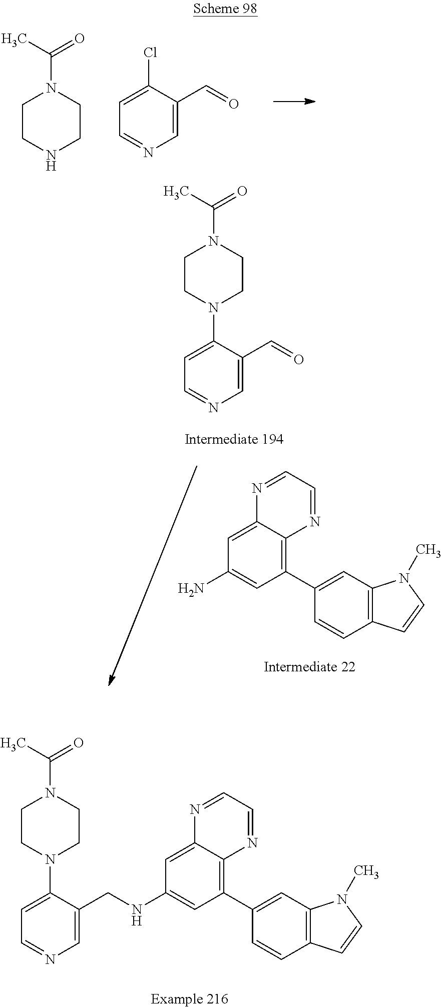

### Intermediate 194

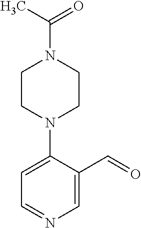 [SMILES: CC(=O)N1CCN(c2ccncc2C=O)CC1]

To a solution of 4-chloropyridine-3-carbaldehyde (44.18 mg; 0.31 mmol; 0.80 eq.), 1-piperazin-1-yl-ethanone (50.00 mg; 0.39 mmol; 1.00 eq.) in EtOH is TEA (86.03 μl; 0.66 mmol; 1.70 eq.) is added. RM is refluxed for 36 h. Purification by FCC (DCM/MeOH; gradient). 4-(4-Acetyl-piperazin-1-yl)-pyridine-3-carbaldehyde (30.00 mg; yield 32.0%; 97% by UPLC) is obtained as a light yellow oil.

### Example 216

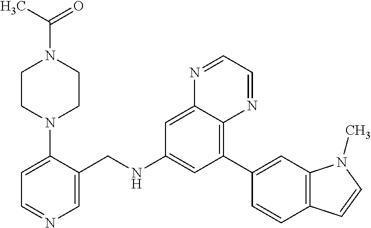 [SMILES: CC(=O)N1CCN(c2ccncc2CNc2cc(-c3ccc4ccn(C)c4c3)c3nccnc3c2)CC1]

The product is prepared according to General Procedure 23, described in Example 63 with 4-(4-acetyl-piperazin-1-yl)-pyridine-3-carbaldehyde (28.75 mg; 0.12 mmol; 1.75 eq.) (Intermediate 194), 8-(1-methyl-1H-indol-6-yl)-quinoxalin-6-ylamine (Intermediate 22) (20.00 mg; 0.07 mmol; 1.00 eq.), Hantzsch ester (26.76 mg; 0.11 mmol; 1.50 eq.), TMCS (2.68 μl; 0.02 mmol; 0.30 eq.) and DCM (5 mL). Purification by FFC (DCM/MeOH; gradient). 1-[4-(3-{[8-(1-Methyl-1H-indol-6-yl)-quinoxalin-6-ylamino]-methyl}-pyridin-4-yl)-piperazin-1-yl]-ethanone (30.00 mg; yield 81.2%; 94% by HPLC) is obtained as a bright yellow solid.

### Example 217

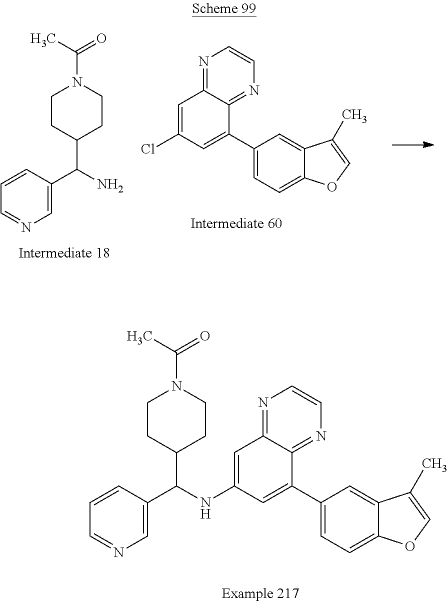 [SMILES: CC(=O)N1CCC(C(N)c2cccnc2)CC1 ; Cc1coc2ccc(-c3cc(Cl)cc4nccnc34)cc12 ; CC(=O)N1CCC(C(Nc2cc(-c3ccc4occ(C)c4c3)c3nccnc3c2)c2cccnc2)CC1]

The product is prepared according to General Procedure 2, described in Example 1 with 7-Chloro-5-(3-methyl-benzofuran-5-yl)-quinoxaline (Intermediate 60) (60.00 mg; 0.16 mmol; 1.00 eq.), 1-[4-(aminopyridin-3-yl-methyl)-piperidin-1-yl]-ethanone (Intermediate 18) (80 mg; 0.30 mmol; 1.8 eq.), NaOtBu (54.96 mg; 0.57 mmol; 3.50 eq.), Pd₂(dba)₃ (15.10 mg; 0.02 mmol; 0.10 eq.), BINAP (10.27 mg; 0.02 mmol; 0.10 eq.) and toluene (5 mL). The reaction mixture was sealed and stirred at 130° C. overnight. Purification by FCC (hexan/EtOAc/MeOH; gradient). 1-(4-{[8-(3-Methyl-benzofuran-5-yl)-quinoxalin-6-ylamino]-pyridin-3-yl-methyl}-piperidin-1-yl)-ethanone (33.00 mg; yield 38.7%; 95% by HPLC) is obtained as a pale yellow powder.

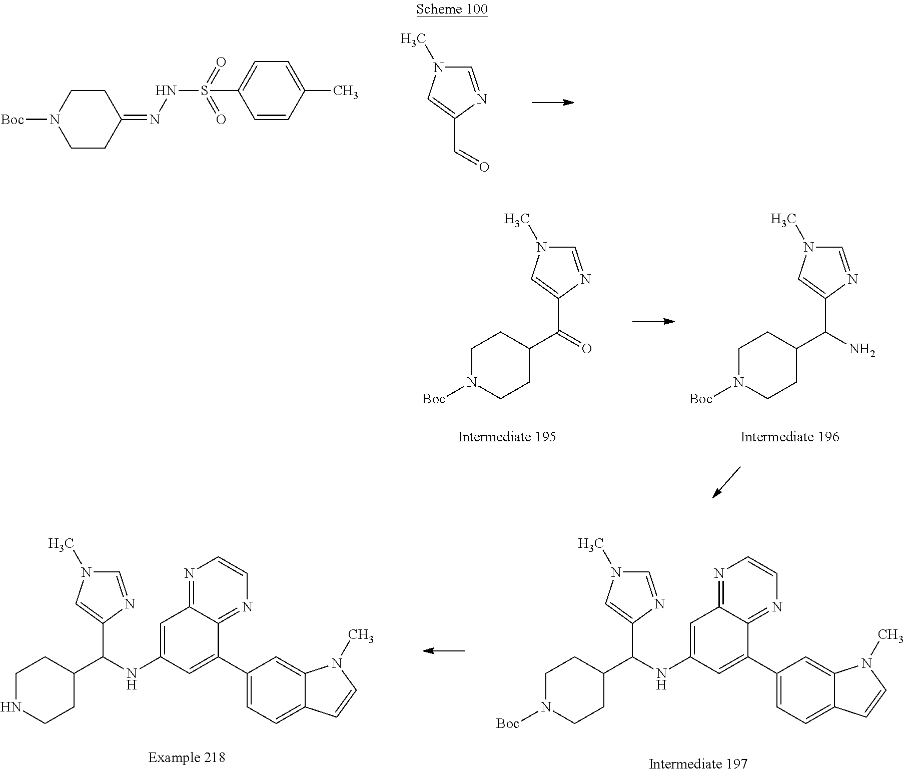

### Intermediate 195—General Procedure 59

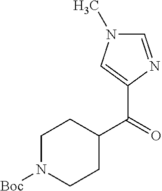 [SMILES: Cn1cnc(C(=O)C2CCN(C(=O)OC(C)(C)C)CC2)c1]

A sealed tube is charged with tert-butyl 4-(p-tolylsulfonylhydrazono)-piperidine-1-carboxylate (1284.79 mg; 3.50 mmol; 1.10 eq.), Cs₂CO₃ (1553.44 mg; 4.77 mmol; 1.50 eq.). The tube is RM is sealed, purged with argone and then 1,4-dioxane (12.00 m) and 1-methyl-00H-imidazole-4-argone and then 1,4-dioxane (12.00 ml-) and 1-methyl-1H-imidazole-4-carbaldehyde (350.00 mg; 3.18 mmol; 1.00 eq.) are added. RM is heated at 110° C. for 48 h. After this time, the mixture is filtered through a Celite® pad and then the solvent is evaporated. Crude product is purified by FCC (hexane/EtOAc; gradient) to afford 4-(1-methyl-1H-imidazole-4-carbonyl)-piperidine-1-carboxylic acid tert-butyl ester (483.90 mg; yield 51.0%; 98% by UPLC) as a beige solid.

### Intermediate 196

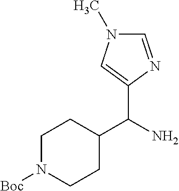 [SMILES: Cn1cnc(C(N)C2CCN(C(=O)OC(C)(C)C)CC2)c1]

The product is prepared according to General Procedure 14, described for Intermediate 12, with 4-(1-methyl-1H-imidazole-4-carbonyl)-piperidine-1-carboxylic acid tert-butyl ester (Intermediate 195) (350.00 mg; 1.17 mmol; 1.00 eq.), TTIP (0.69 ml; 2.35 mmol; 2.00 eq.), NaBH₄ (177.48 mg; 4.69 mmol; 4.00 eq.) and 7M NH₃ in MeOH (6.70 ml; 46.91 mmol; 40.00 eq.). Crude 4-[amino-(1-methyl-1H-imidazol-4-yl)-methyl]-piperidine-1-carboxylic acid tert-butyl ester (330.00 mg; yield 76.5%; colorless oil) is used in the next step without purification.

### Intermediate 197

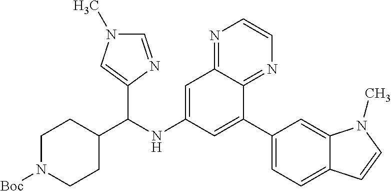 [SMILES: Cn1cnc(C(Nc2cc(-c3ccc4ccn(C)c4c3)c3nccnc3c2)C2CCN(C(=O)OC(C)(C)C)CC2)c1]

The product is prepared according to General Procedure 2, described in Example 1 with 7-chloro-5-(1-methyl-1H-indol-6-yl)-quinoxaline (Intermediate 4) (48.00 mg; 0.16 mmol; 1.00 eq.), 4-[amino-(1-methyl-1H-imidazol-4-yl)-methyl]-piperidine-1-carboxylic acid tert-butyl ester (Intermediate 196) (149.83 mg; 0.41 mmol; 1.00 eq.), NaOtBu (58.69 mg; 0.61 mmol; 1.50 eq.), Pd₂(dba)₃ (16.91 mg; 0.02 mmol; 0.10 eq.), BINAP (5.07 mg; 0.01 mmol; 0.02 eq.) and toluene (5 mL). The reaction mixture was sealed and stirred at 120° C. for 48 h. Purification by FCC (hexane/EtOAc/MeOH; gradient). 4-{(1-Methyl-1H-imidazol-4-yl)-[8-(1-methyl-1H-indol-6-yl)-quinoxalin-6-ylamino]-methyl}-piperidine-1-carboxylic acid tert-butyl ester (98.00 mg; yield 41.4%; 94% by UPLC) is obtained as an orange powder.

### Example 218

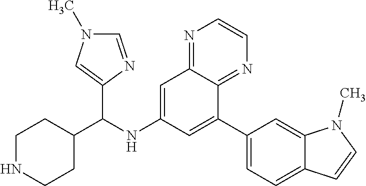 [SMILES: Cn1cnc(C(Nc2cc(-c3ccc4ccn(C)c4c3)c3nccnc3c2)C2CCNCC2)c1]

The product is prepared according to General Procedure 10, described in Example 44 with 4-{(1-methyl-1H-imidazol-4-yl)-[8-(1-methyl-1H-indol-6-yl)-quinoxalin-6-ylamino]-methyl}-piperidine-1-carboxylic acid tert-butyl ester (Intermediate 197) (85.00 mg; 0.15 mmol; 1.00 eq.) and TFA (3 mL). Purification by FCC (EtOAc/MeOH; gradient) and repurification by HPLC. [(1-Methyl-1H-imidazol-4-yl)-piperidin-4-yl-methyl]-[8-(1-methyl-1H-indol-6-yl)-quinoxalin-6-yl]-amine (5.00 mg; yield 7.2%; 100% by HPLC) is obtained as an orange powder.

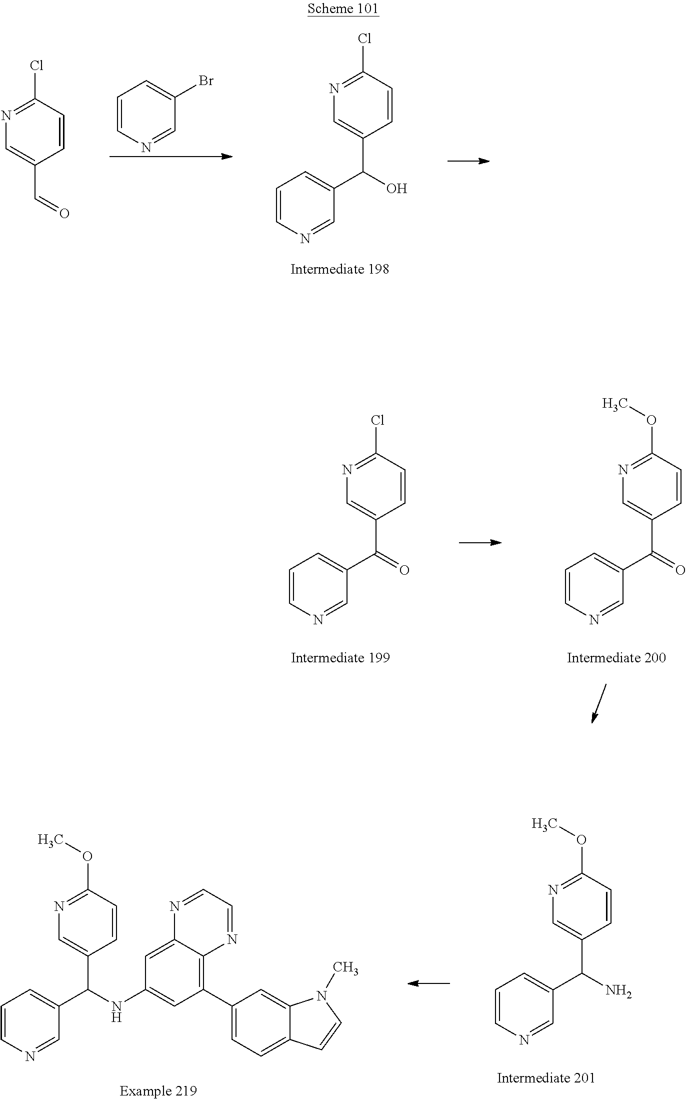

### Intermediate 198—General Procedure 60

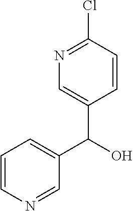 [SMILES: OC(c1cccnc1)c1ccc(Cl)nc1]

To a dry flask containing 3-bromo-pyridine (8.37 g; 52.98 mmol; 2.50 eq.) is added anhydrous THF (15 mL). The solution is sparged with argon for 30 min and then isopropylmagnesium chloride/LiCl solution 1.3 M in THF (40.76 ml; 52.98 mmol; 2.50 eq.) is added dropwise to the reaction flask over a period of 10 min. After stirring for 4 h at RT the resulting solution of 3-pyridylmagnesium bromide is added dropwise into another solution of 6-chloro-pyridine-3-carbaldehyde (3.00 g; 21.19 mmol; 1.00 eq.) in THF (50 mL) at 0° C. and the resulting mixture is stirred overnight. RM is quenched with NH₄Cl (100 mL) and extracted with ethyl acetate (2×200 mL). The organic layer is washed with brine (200 mL), dried over MgSO4, filtered and concentrated in vacuo to obtain: (6-chloro-pyridin-3-yl)-pyridin-3-yl-methanol (7.00 g; yield 106.3%; 71% by UPLC; brown oil).

### Intermediate 199

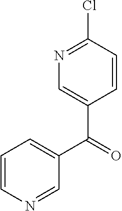 [SMILES: O=C(c1cccnc1)c1ccc(Cl)nc1]

Well grounded mixture of chloro-oxido-dioxo-chromium pyridine (9.02 g; 41.83 mmol; 2.00 eq.) and SiO₂ gel (9 g) is added to a solution of (6-chloro-pyridin-3-yl)-pyridin-3-yl-methanol (Intermediate 198) (6.50 g; 20.92 mmol; 1.00 eq.) in DCM (100 mL). After stirring for 15 minutes, RM is filtered and concentrated in vacuo to give crude product as brown oil (6 g). Purification by FCC (DCM/MeOH) provided (6-chloro-pyridin-3-yl)-pyridin-3-yl-methanone (1.78 g; yield 38.5%; 99% by UPLC; off-white solid).

### Intermediate 200

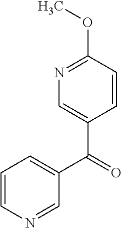 [SMILES: COc1ccc(C(=O)c2cccnc2)cn1]

A solution of (6-chloro-pyridin-3-yl)-pyridin-3-yl-methanone (Intermediate 199) (350.00 mg; 1.58 mmol; 1.00 eq.) and sodium methanolate (6.34 ml; 3.17 mmol; 2.00 eq.) in MeOH is stirred at 65° C. overnight. After this time the solvent is evaporated, residue is dissolved in water (6 mL) and the mixture is extracted with EtOAc. The organic layer is dried over Na₂SO₄, filtered and concentrated in vacuo. (6-Methoxy-pyridin-3-yl)-pyridin-3-yl-methanone (340.00 mg; yield 98.1%; 98% by UPLC) is obtained as an clear gum.

### Intermediate 201

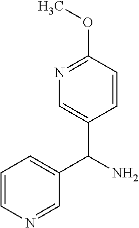 [SMILES: COc1ccc(C(N)c2cccnc2)cn1]

The product is prepared according to General Procedure 14, described for Intermediate 12, with (6-methoxy-pyridin-3-yl)-pyridin-3-yl-methanone (Intermediate 200) (340.00 mg; 1.56 mmol; 1.00 eq.), TTIP (0.92 ml; 3.11 mmol; 2.00 eq.), NaBH₄ (235.38 mg; 6.22 mmol; 4.00 eq.) and 7M NH₃ in MeOH (8.13 ml; 56.94 mmol; 36.61 eq.). Crude C-(6-methoxy-pyridin-3-yl)-C-pyridin-3-yl-methylamine (276.00 mg; yield 75.0%; yellow oil; 91% by UPLC) is used in the next step without purification.

### Example 219

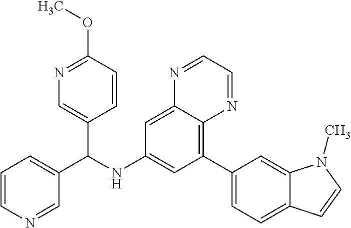 [SMILES: COc1ccc(C(Nc2cc(-c3ccc4ccn(C)c4c3)c3nccnc3c2)c2cccnc2)cn1]

The product is prepared according to General Procedure 6, described for Intermediate 6 with 7-chloro-5-(1-methyl-1H-indol-6-yl)-quinoxaline (Intermediate 4) (313.73 mg; 1.01 mmol; 1.00 eq.), C-(6-methoxy-pyridin-3-yl)-C-pyridin-3-yl-methylamine (Intermediate 201) (240.00 mg; 1.01 mmol; 1.00 eq.), Cs₂CO₃ (1001.78 mg; 3.04 mmol; 3.00 eq.), BINAP (64.47 mg; 0.10 mmol; 0.10 eq.), Pd(OAc)₂ (23.98 mg; 0.10 mmol; 0.10 eq.) and 1,4-dioxane (6.00 mL). RM is stirred for 2h at 130° C. Purification by HPLC. [(6-Methoxy-pyridin-3-yl)-pyridin-3-yl-methyl]-[8-(1-methyl-1H-indol-6-yl)-quinoxalin-6-yl]-amine (132.00 mg; yield 27.3%; 95% by HPLC) is obtained as a yellow solid.

### Example 220

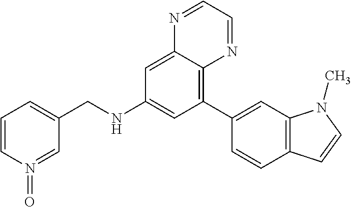 [SMILES: Cn1ccc2ccc(-c3cc(NCc4ccc[n+]([O-])c4)cc4nccnc34)cc21]

The product is prepared according to General Procedure 6, described for Intermediate 6 with 7-chloro-5-(1-methyl-1H-indol-6-yl)-quinoxaline (60.00 mg; 0.20 mmol; 1.00 eq.), C-(1-oxy-pyridin-3-yl)-methylamine hydrochloride (37.63 mg; 0.23 mmol; 1.20 eq.), Cs₂CO₃ (154.24 mg; 0.47 mmol; 2.40 eq.), BINAP (12.80 mg; 0.02 mmol; 0.10 eq.), Pd(OAc)₂ (4.61 mg; 0.02 mmol; 0.10 eq.) and 1,4-dioxane (2 mL). RM is stirred for 1 h at 150° C. Purification by FCC (EtOAc/MeOH; gradient). [8-(1-Methyl-1H-indol-6-yl)-quinoxalin-6-yl]-(1-oxy-pyridin-3-ylmethyl)-amine (20.00 mg; yield 23.9%; 89% by HPLC) is obtained as a yellow-brown powder

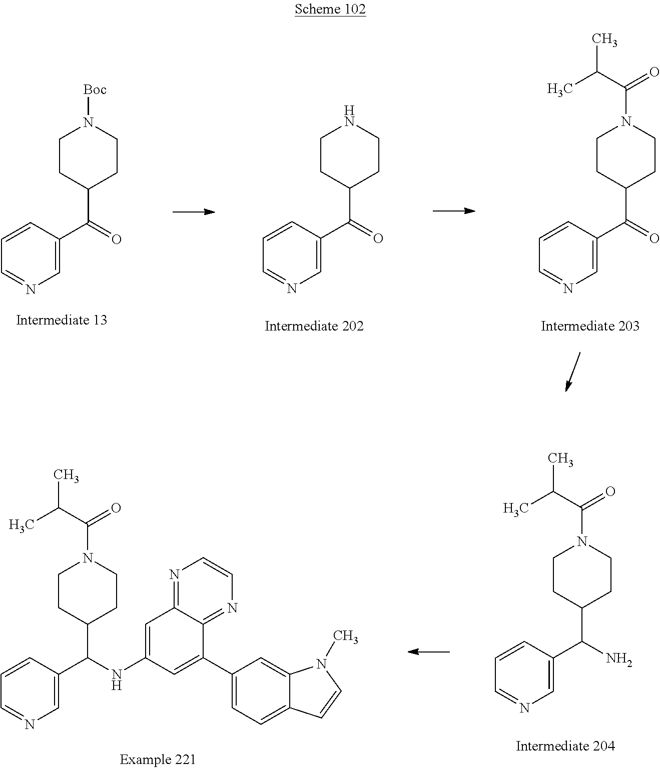 [SMILES: CC(C)(C)OC(=O)N1CCC(C(=O)c2cccnc2)CC1 ; O=C(c1cccnc1)C1CCNCC1 ; CC(C)C(=O)N1CCC(C(=O)c2cccnc2)CC1 ; CC(C)C(=O)N1CCC(C(Nc2cc(-c3ccc4ccn(C)c4c3)c3nccnc3c2)c2cccnc2)CC1 ; CC(C)C(=O)N1CCC(C(N)c2cccnc2)CC1]

### Intermediate 202

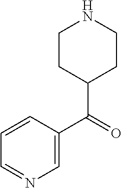 [SMILES: O=C(c1cccnc1)C1CCNCC1]

The product is prepared according to General Procedure 10, described in Example 44 with 4-(pyridine-4-carbonyl)-piperidine-1-carboxylic acid tert-butyl ester (Intermediate 13) (750.00 mg; 2.32 mmol; 1.00 eq.) and TFA (2.50 mL). Purification by FCC (DCM/MeOH; gradient). Piperidin-4-yl-pyridin-3-yl-methanone (360.00 mg; yield 81.4%; 100% by UPLC) is obtained as a yellow solid.

### Intermediate 202

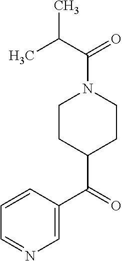 [SMILES: CC(C)C(=O)N1CCC(C(=O)c2cccnc2)CC1]

The product is prepared according to General Procedure 35, described in Example 82 with piperidin-4-yl-pyridin-3-yl-methanone (Intermediate 201) (80.00 mg; 0.42 mmol; 1.00 eq.), isobutyryl chloride (49.29 mg; 0.46 mmol; 1.10 eq.), TEA (136.38 μl; 1.05 mmol; 2.50 eq.) and DCM anhydrous (10.00 mL). Crude 2-methyl-1-[4-(pyridine-3-carbonyl)-piperidin-1-yl]-propan-1-one (100.00 mg; yield 86.4%; 94% by UPLC; yellow oil) is used in the next step, without purification.

### Intermediate 203

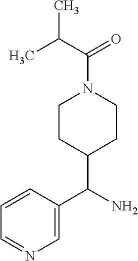 [SMILES: CC(C)C(=O)N1CCC(C(N)c2cccnc2)CC1]

The product is prepared according to General Procedure 14, described for Intermediate 12, with 2-methyl-1-[4-(pyridine-3-carbonyl)-piperidin-1-yl]-propan-1-one (Intermediate 202) (100.00 mg; 0.35 mmol; 1.00 eq.), TTIP (0.20 ml; 0.69 mmol; 2.00 eq.), NaBH₄ (52.32 mg; 1.38 mmol; 4.00 eq.) and 7M NH₃ in MeOH (10 mL). Crude 1-[4-(amino-pyridin-3-yl-methyl)-piperidin-1-yl]-2-methyl-propan-1-one (50.00 mg; yield 52.0%; white-yellow solid; 94% by UPLC) is used in the next step, without purification.

### Example 221

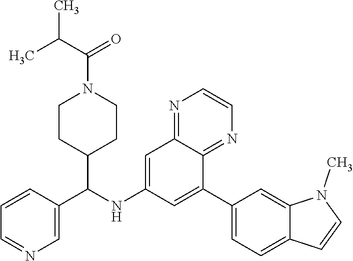 [SMILES: CC(C)C(=O)N1CCC(C(Nc2cc(-c3ccc4ccn(C)c4c3)c3nccnc3c2)c2cccnc2)CC1]

The product is prepared according to General Procedure 2, described in Example 1, with 7-chloro-5-(1-methyl-1H-indol-6-yl)-quinoxaline (Intermediate 4) (50.00 mg; 0.17 mmol; 1.00 eq.), 1-[4-(amino-pyridin-3-yl-methyl)-piperidin-1-yl]-2-methyl-propan-1-one (Intermediate 203) (66.73 mg; 0.26 mmol; 1.50 eq.), NaOtBu (57.25 mg; 0.60 mmol; 3.50 eq.), BINAP (21.20 mg; 0.03 mmol; 0.20 eq.), Pd₂(dba)₃ (15.59 mg; 0.02 mmol; 0.10 eq.) and toluene (5.00 mL). RM is stirred at 120° C. overnight. Purification by FCC (DCM/MeOH; gradient). 2-Methyl-1-(4-{[8-(1-methyl-1H-indol-6-yl)-quinoxalin-6-ylamino]-pyridin-3-yl-methyl}-piperidin-1-yl)-propan-1-one (20.00 mg; yield 22.2%; 97% by HPLC) is obtained as a yellow powder.

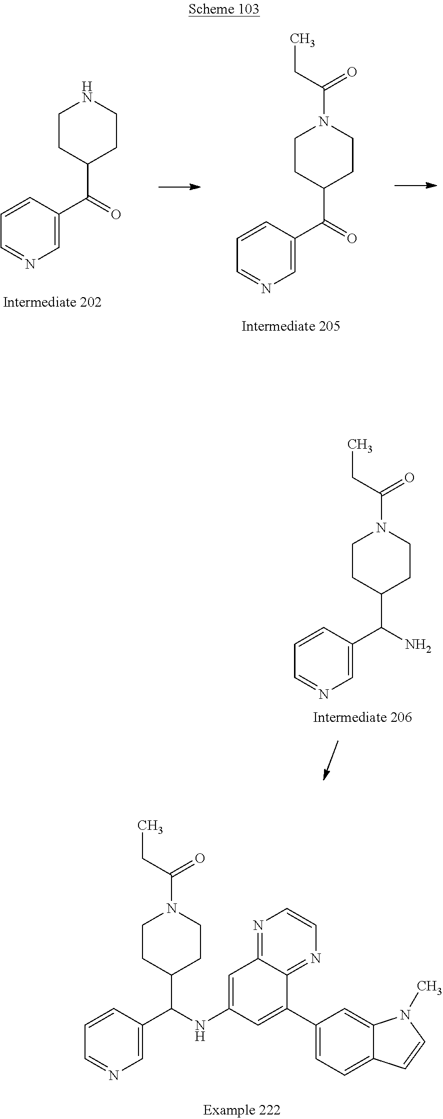 [SMILES: O=C(c1cccnc1)C1CCNCC1 ; CCC(=O)N1CCC(C(=O)c2cccnc2)CC1 ; CCC(=O)N1CCC(C(N)c2cccnc2)CC1 ; CCC(=O)N1CCC(C(Nc2cc(-c3ccc4ccn(C)c4c3)c3nccnc3c2)c2cccnc2)CC1]

### Intermediate 205

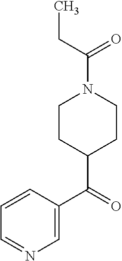 [SMILES: CCC(=O)N1CCC(C(=O)c2cccnc2)CC1]

The product is prepared according to General Procedure 35, described in Example 82 with piperidin-4-yl-pyridin-3-yl-methanone (Intermediate 202) (80.00 mg; 0.42 mmol; 1.00 eq.), propanoyl propanoate (60.20 mg; 0.46 mmol; 1.10 eq.), TEA (136.38 μl; 1.05 mmol; 2.50 eq.) and DCM anhydrous (10.00 mL). Crude 1-[4-(pyridine-3-carbonyl)-piperidin-1-yl]-propan-1-one (100.00 mg; yield 92.7%; 96% by UPLC) is used in the next step, without purification.

### Intermediate 206

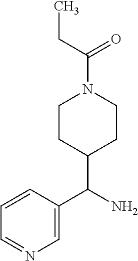 [SMILES: CCC(=O)N1CCC(C(N)c2cccnc2)CC1]

The product is prepared according to General Procedure 14, described for Intermediate 12, with 1-[4-(pyridine-3-carbonyl)-piperidin-1-yl]-propan-1-one (Intermediate 205) (100.00 mg; 0.37 mmol; 1.00 eq.), TTIP (0.22 ml; 0.73 mmol; 2.00 eq.), NaBH₄ (55.30 mg; 1.46 mmol; 4.00 eq.) and 7M NH₃ in MeOH (10 mL). Crude 1-[4-(amino-pyridin-3-yl-methyl)-piperidin-1-yl]-propan-1-one (70.00 mg; yield 73.9%; 82% by UPLC) is used in the next step, without purification.

### Example 222

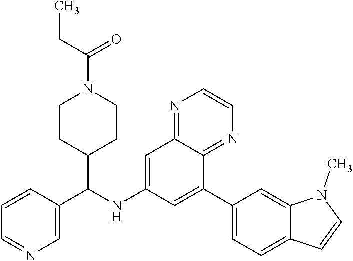 [SMILES: CCC(=O)N1CCC(C(Nc2cc(-c3ccc4ccn(C)c4c3)c3nccnc3c2)c2cccnc2)CC1]

The product is prepared according to General Procedure 2, described in Example 1, with 7-chloro-5-(1-methyl-1H-indol-6-yl)-quinoxaline (Intermediate 4) (50.00 mg; 0.17 mmol; 1.00 eq.), 1-[4-(amino-pyridin-3-yl-methyl)-piperidin-1-yl]-propan-1-one (Intermediate 206) (63.15 mg; 0.26 mmol; 1.50 eq.), NaOtBu (57.25 mg; 0.60 mmol; 3.50 eq.), BINAP (21.20 mg; 0.03 mmol; 0.20 eq.), Pd₂(dba)₃ (15.59 mg; 0.02 mmol; 0.10 eq.) and toluene (5.00 mL). RM is stirred at 120° C. overnight. Purification by FCC (Hexane/EtOAc/MeOH; gradient). 1-(4-{[8-(1-Methyl-1H-indol-6-yl)-quinoxalin-6-ylamino]-pyridin-3-yl-methyl}-piperidin-1-yl)-propan-1-one (60.00 mg; yield 67.2%; 96% by UPLC) is obtained as a yellow powder.

### Example 223—General Procedure 61

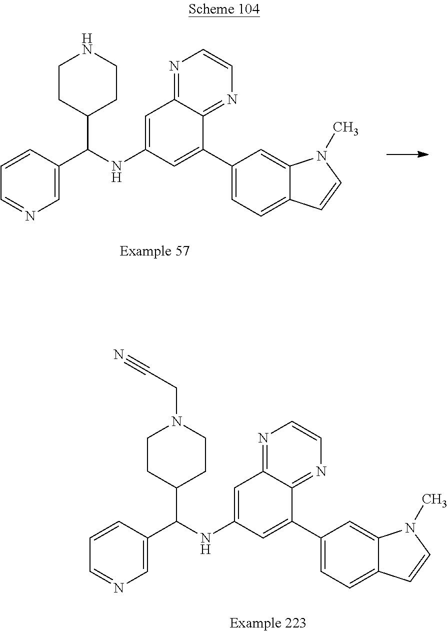 [SMILES: Cn1ccc2ccc(-c3cc(NC(c4cccnc4)C4CCNCC4)cc4nccnc34)cc21 ; Cn1ccc2ccc(-c3cc(NC(c4cccnc4)C4CCN(CC#N)CC4)cc4nccnc34)cc21]

Bromo-acetonitrile (8.12 μL; 0.12 mmol; 1.10 eq.) is added to a solution of [8-(1-methyl-1H-indol-6-yl)-quinoxalin-6-yl]-(piperidin-4-yl-pyridin-3-yl-methyl)-amine (Example 57) (60.00 mg; 0.11 mmol; 1.00 eq.) and K₂CO₃ (17.97 mg; 0.13 mmol; 1.20 eq.) in CH₃CN. The reaction mixture is stirred for 2h at 0° C. The reaction is quenched with NaHCO₃ and extracted with EtOAc. Purification by FCC (DCM/MeOH; gradient). (4-{[8-(1-Methyl-1H-indol-6-yl)-quinoxalin-6-ylamino]-pyridin-3-yl-methyl}-piperidin-1-yl)-acetonitrile (25.00 mg; yield 45.2%; 95% by HPLC) is obtained as a yellow powder.

### Example 224

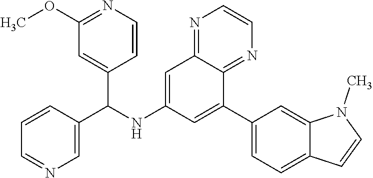 [SMILES: COc1cc(C(Nc2cc(-c3ccc4ccn(C)c4c3)c3nccnc3c2)c2cccnc2)ccn1]

The product is prepared according to General Procedure 6, described for Intermediate 6 with 7-chloro-5-(1-methyl-1H-indol-6-yl)-quinoxaline (Intermediate 4) (150.00 mg; 0.49 mmol; 1.00 eq.), C-(2-methoxy-pyridin-4-yl)-C-pyridin-3-yl-methylamine (184.27 mg; 0.73 mmol; 1.50 eq.), Cs₂CO₃ (632.23 mg; 1.94 mmol; 4.00 eq.), BINAP (30.21 mg; 0.05 mmol; 0.10 eq.) and Pd(OAc)₂ (10.89 mg; 0.05 mmol; 0.10 eq.) and 1,4-dioxane (2 mL). RM is stirred for 2h at 130° C. Purification by FCC (DCM/MeOH: gradient). [(2-Methoxy-pyridin-4-yl)-pyridin-3-yl-methyl]-[8-(1-methyl-1H-indol-6-yl)-quinoxalin-6-yl]-amine (80.00 mg; yield 31.9%; 91% by HPLC) is obtained as a yellow solid.

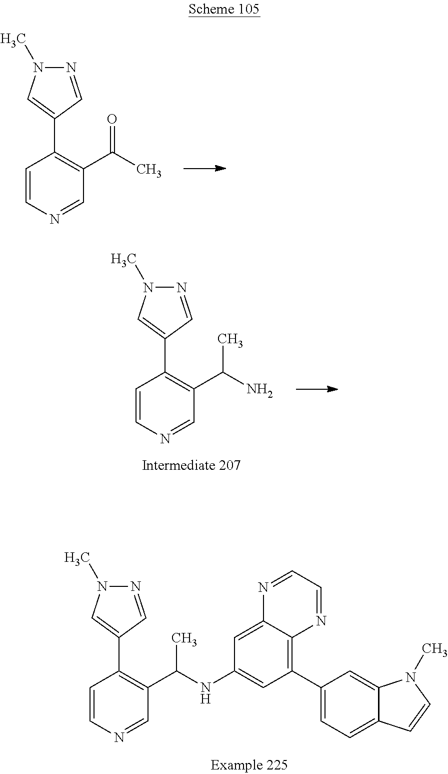 [SMILES: CC(=O)c1cnccc1-c1cnn(C)c1 ; CC(N)c1cnccc1-c1cnn(C)c1 ; CC(Nc1cc(-c2ccc3ccn(C)c3c2)c2nccnc2c1)c1cnccc1-c1cnn(C)c1]

### Intermediate 207

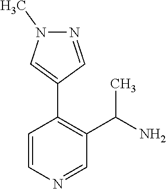

The product is prepared according to General Procedure 14, described for Intermediate 12, with 1-[4-(1-methyl-1H-pyrazol-4-yl)-pyridin-3-yl]-ethanone (86.40 mg; 0.42 mmol; 1.00 eq.), TTIP (0.25 ml; 0.84 mmol; 2.00 eq.), NaBH₄ (63.87 mg; 1.69 mmol; 4.00 eq.) and 7M NH₃ in MeOH (2.41 ml; 16.88 mmol; 40.00 eq.). Purification by FCC (Al₂O₃; DCM/MeOH; gradient). 1-[4-(1-Methyl-1H-pyrazol-4-yl)-pyridin-3-yl]-ethylamine (59.90 mg; yiled 62.5%; 89% by UPLC) is obtained as a colorless oil.

### Example 225

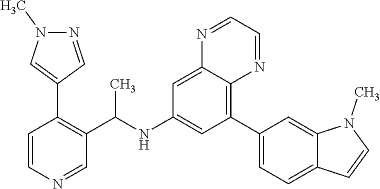 [SMILES: CC(Nc1cc(-c2ccc3ccn(C)c3c2)c2nccnc2c1)c1cnccc1-c1cnn(C)c1]

The product is prepared according to General Procedure 2, described in Example 1 with 7-chloro-5-(1-methyl-1H-indol-6-yl)-quinoxaline (Intermediate 4) (84.25 mg; 0.26 mmol; 1.00 eq.), 1-[4-(1-methyl-1H-pyrazol-4-yl)-pyridin-3-yl]-ethylamine (Intermediate 207) (59.90 mg; 0.26 mmol; 1.00 eq.), NaOtBu (38.04 mg; 0.40 mmol; 1.50 eq.), BINAP (3.29 mg; 0.01 mmol; 0.02 eq.) and Pd₂(dba)₃ (2.42 mg; 0.00 mmol; 0.01 eq.) and 1,4-dioxane (2.00 mL). RM is stirred at 120° C. for 48 h. Purification by FCC (hexane/EtOAc/MeOH; gradient). [8-(1-Methyl-1H-indol-6-yl)-quinoxalin-6-yl]-{1-[4-(1-methyl-1H-pyrazol-4-yl)-pyridin-3-yl]-ethyl}-amine (52.00 mg; yield 41.9%; 97% by HPLC) is obtained as a yellow solid).

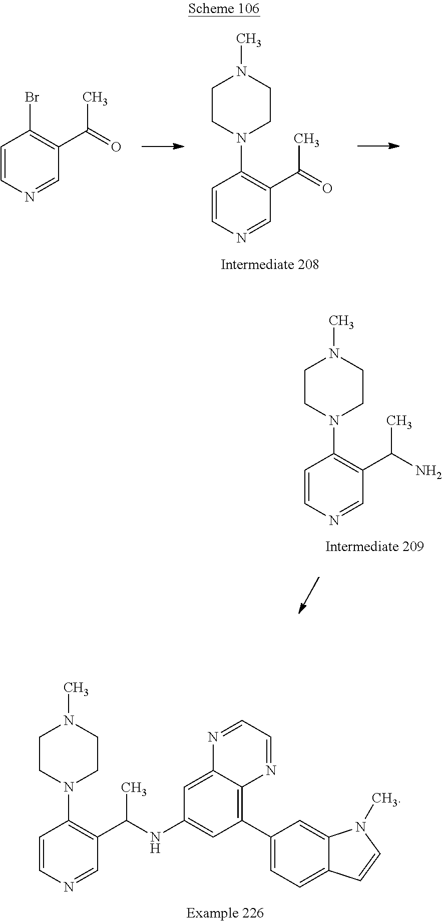

### Intermediate 208

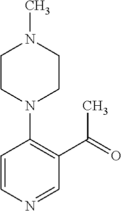 [SMILES: CC(=O)c1cnccc1N1CCN(C)CC1]

The product is prepared according to General Procedure 6, described for Intermediate 6 with 1-(4-bromo-pyridin-3-yl)-ethanone hydrobromide (150.00 mg; 0.53 mmol; 1.00 eq.), 1-methyl-piperazine (64.17 mg; 0.9 mmol; 1.2 eq.), Cs₂CO₃ (695.84 mg; 2.14 mmol; 4.00 eq.), BINAP (33.25 mg; 0.05 mmol; 0.10 eq.), Pd(OAc)₂ (11.99 mg; 0.05 mmol; 0.10 eq.) and 1,4-dioxane (3 mL). RM is stirred for 1h at 100° C. Purification by FCC (hexane/EtOAc; gradient). 1-[4-(4-Methyl-piperazin-1-yl)-pyridin-3-yl]-ethanone (68.40 mg; yield 32.1%; 55% by UPLC) is obtained as a colourless oil.

### Intermediate 209

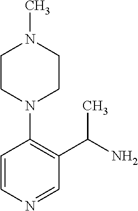 [SMILES: CC(N)c1cnccc1N1CCN(C)CC1]

The product is prepared according to General Procedure 14, described for Intermediate 12, with 1-[4-(4-methyl-piperazin-1-yl)-pyridin-3-yl]-ethanone (Intermediate 208) (60.00 mg; 0.27 mmol; 1.00 eq.), TTIP (0.16 ml; 0.54 mmol; 2.00 eq.), NaBH₄ (40.70 mg; 1.08 mmol; 4.00 eq.) and 7M NH₃ in MeOH (1.54 ml; 10.76 mmol; 40.00 eq.). Purification by FCC (Al₂O₃; DCM/MeOH; gradient). 1-[4-(4-Methyl-piperazin-1-yl)-pyridin-3-yl]-ethylamine (19.90 mg; yield 26.9%; 80% by UPLC) is obtained as a colorless oil.

### Example 226

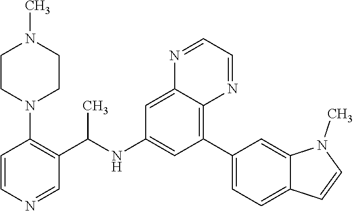 [SMILES: CC(Nc1cc(-c2ccc3ccn(C)c3c2)c2nccnc2c1)c1cnccc1N1CCN(C)CC1]

The product is prepared according to General Procedure 2, described in Example 1 with 7-chloro-5-(1-methyl-1H-indol-6-yl)-quinoxaline (Intermediate 4) (23.07 mg; 0.07 mmol; 1.00 eq.), 1-[4-(4-methyl-piperazin-1-yl)-pyridin-3-yl]-ethylamine (Intermediate 209) (19.90 mg; 0.07 mmol; 1.00 eq.), NaOtBu (10.42 mg; 0.11 mmol; 1.50 eq.), BINAP (0.90 mg; 0.001 mmol; 0.02 eq.), Pd₂(dba)₃ (0.66 mg; 0.00 mmol; 0.01 eq.) and 1,4-dioxane (2 mL). RM is stirred at 110° C. for 48 h. Purification by FCC (hexane/EtOAc/MeOH; gradient). [8-(1-Methyl-1H-indol-6-yl)-quinoxalin-6-yl]-{1-[4-(4-methyl-piperazin-1-yl)-pyridin-3-yl]-ethyl}-amine (15.10 mg; yield 42.7%; 97% by HPLC) is obtained as a yellow powder.

### Example 227

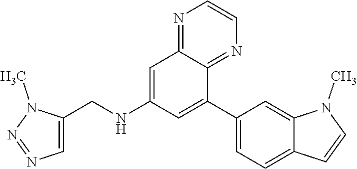 [SMILES: Cn1nncc1CNc1cc(-c2ccc3ccn(C)c3c2)c2nccnc2c1]

The product is prepared according to General Procedure 23, described in Example 63 with 3-methyl-3H-[1,2,3]triazole-4-carbaldehyde (16.84 mg; 0.15 mmol; 1.00 eq.), 8-(1-methyl-1H-indol-6-yl)-quinoxalin-6-ylamine (Intermediate 22) (42.00 mg; 0.15 mmol; 1.00 eq.), Hantzsch ester (66.90 mg; 0.26 mmol; 1.50 eq.), TMCS (3.85 μl; 0.03 mmol; 0.20 eq.) and DCM (4 mL). Purification by FFC (hexane/EtOAc/MeOH; gradient). [8-(1-Methyl-1H-indol-6-yl)-quinoxalin-6-yl]-(3-methyl-3H-[1,2, 3]triazol-4-ylmethyl)-amine (35.00 mg; yield 58.9%; 94% by HPLC) is obtained as a yellow powder.

### Example 228

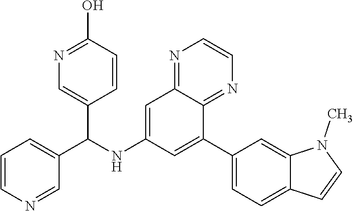 [SMILES: Cn1ccc2ccc(-c3cc(NC(c4cccnc4)c4ccc(O)nc4)cc4nccnc34)cc21]

The suspension of [(6-methoxy-pyridin-3-yl)-pyridin-3-yl-methyl]-[8-(1-methyl-1H-indol-6-yl)-quinoxalin-6-yl]-amine (Example 219) (500.00 mg; 1.05 mmol; 1.00 eq.) and methylsulfanylsodium (220.26 mg; 3.14 mmol; 3.00 eq.) in DMF (10.00 mL) is stirred for 48 h at 60° C. Acetic acid (3.0 ml) is added, and the mixture is evaporated to dryness. The yellow oily residue is purified by HPLC. 5-{[8-(1-methyl-1H-indol-6-yl)-quinoxalin-6-ylamino]-pyridin-3-yl-methyl}-pyridin-2-ol (233.00 mg; yield 46.1%; 95% by HPLC) is obtained as as orange solid.

### Example 229

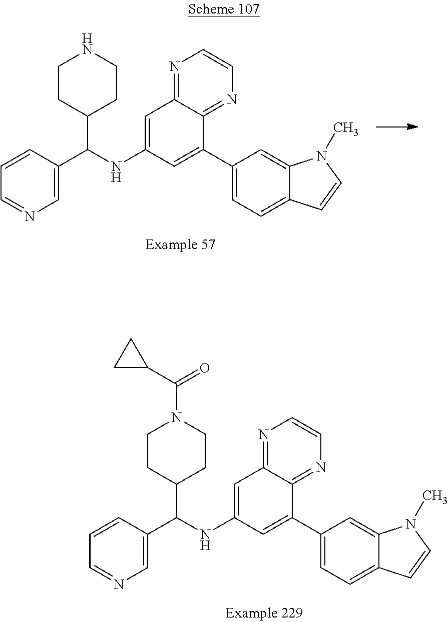 [SMILES: Cn1ccc2ccc(-c3cc(NC(c4cccnc4)C4CCNCC4)cc4nccnc34)cc21 ; Cn1ccc2ccc(-c3cc(NC(c4cccnc4)C4CCN(C(=O)C5CC5)CC4)cc4nccnc34)cc21]

The product is prepared according to General Procedure 35, described in Example 82 with [8-(1-methyl-1H-indol-6-yl)-quinoxalin-6-yl]-(piperidin-4-yl-pyridin-3-yl-methyl)-amine (Example 57) (70.00 mg; 0.16 mmol; 1.00 eq.), cyclopropanecarbonyl chloride (15.58 μl; 0.17 mmol; 1.10 eq.), TEA (50.61 μl; 0.39 mmol; 2.50 eq.) and DCM anhydrous (10.00 mL). Purification by FCC (DCM/MeOH; gradient). Cyclopropyl-(4-{[8-(1-methyl-1H-indol-6-yl)-quinoxalin-6-ylamino]-pyridin-3-yl-methyl}-piperidin-1-yl)-methanone (20.00 mg; yield 23.9%; 96% by HPLC) is obtained as a yellow powder.

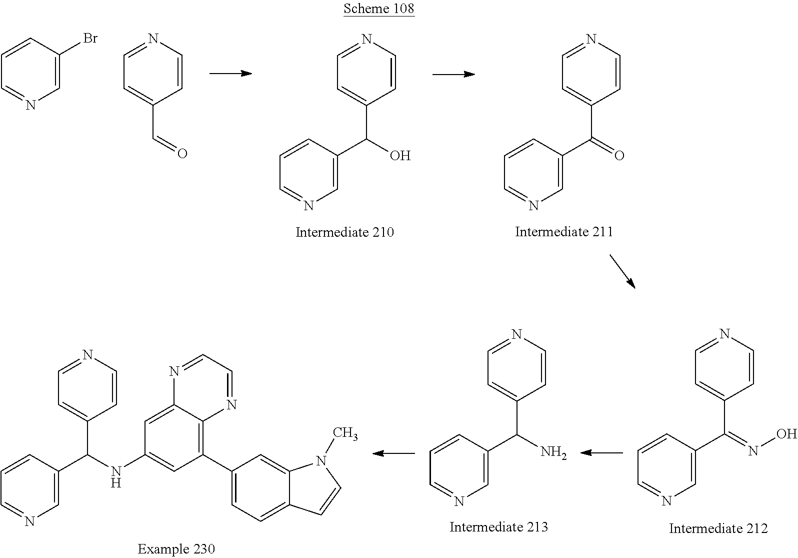

### Intermediate 210

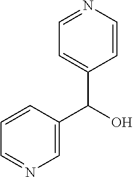 [SMILES: OC(c1ccncc1)c1cccnc1]

The product is prepared according to General Procedure 60, described for Intermediate 198 with 3-bromo-pyridine (346.15 μL; 3.50 mmol; 2.50 eq.) in THF (15 mL), isopropylmagnesium chloride/LiCl solution 1.3 M in THF (2.69 mL; 3.50 mmol; 2.50 eq.) and pyridine-4-carbaldehyde (132.16 μL; 1.40 mmol; 1.00 eq.) in THF (15.00 mL). Crude pyridin-3-yl-pyridin-4-yl-methanol (350.00 mg; yield 2.1%; amorphous yellow solid) is used in the next step without purification.

### Intermediate 211—General Procedure 62

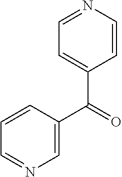 [SMILES: O=C(c1ccncc1)c1cccnc1]

To a solution of pyridin-3-yl-pyridin-4-yl-methanol (Intermediate 210) (350.00 mg; 1.41 mmol; 1.00 eq.) in THF (5 mL) MnO₂ (0.87 g; 2.82 mmol; 2.00 eq.) is added. RM is stirred at 70° C. overnight and reaction is filtrated by Celite® and concentrated. Crude pyridin-3-yl-pyridin-4-yl-methanone (400.00 mg; 52.7%; white solid) is directly used in the next step.

### Intermediate 212

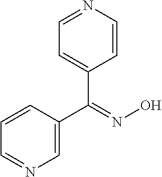 [SMILES: O/N=C(\c1ccncc1)c1cccnc1]

The product is prepared according to General Procedure 17, described for Intermediate 19 with pyridin-3-yl-pyridin-4-yl-methanone (Intermediate 211) (400.00 mg; 2.17 mmol; 1.00 eq.), hydroxylamine hydrochloride (377.27 mg; 5.43 mmol; 2.50 eq.), NaOAc (445.37 mg; 5.43 mmol; 2.50 eq.) and MeOH. The mixture is stirred at 80° C. for 2h. Solvent is evaporated. Crude product is used in the next step.

### Intermediate 213

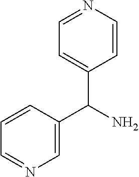 [SMILES: NC(c1ccncc1)c1cccnc1]

The product is prepared according to General Procedure 18, described for Intermediate 20 with pyridin-3-yl-pyridin-4-yl-methanone oxime (Intermediate 212) (400.00 mg; 2.01 mmol; 1.00 eq.), NH₄OAc (232.16 mg; 3.01 mmol; 1.50 eq.), zinc dust (656.50 mg; 10.04 mmol; 5.00 eq.), ethanol (5 mL), water (5 mL) and ammonia 25% solution in water (5 mL). Crude C-pyridin-4-yl-C-pyridin-3-yl-methylamine (365.00 mg; 1.42 mmol; 70.6%; beige solid) is used in the next step, without purification.

### Example 230

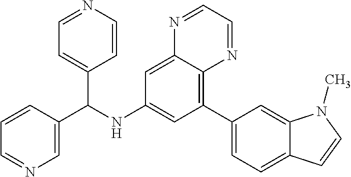 [SMILES: Cn1ccc2ccc(-c3cc(NC(c4ccncc4)c4cccnc4)cc4nccnc34)cc21]

The product is prepared according to General Procedure 2, described in Example 1 with C-pyridin-3-yl-C-pyridin-4-yl-methylamine (Intermediate 213) (94.58 mg; 0.51 mmol; 1.50 eq.), 7-chloro-5-(1-methyl-1H-indol-6-yl)-quinoxaline (Intermediate 4) (100.00 mg; 0.34 mmol; 1.00 eq.), NaOtBu (65.43 mg; 0.68 mmol; 2.00 eq.), Pd₂(dba)₃ (15.59 mg; 0.02 mmol; 0.05 eq.), BINAP (31.80 mg; 0.05 mmol; 0.15 eq.) and toluene (5 mL). Purification by FCC (DCM:MeOH; gradient), repurification by preparative HPLC. [8-(1-Methyl-1H-indol-6-yl)-quinoxalin-6-yl]-(pyridin-3-yl-pyridin-4-yl-methyl)-amine (10.00 mg; yield 6.5%; 97% by HPLC) is obtained as a yellow solid.

### Example 231

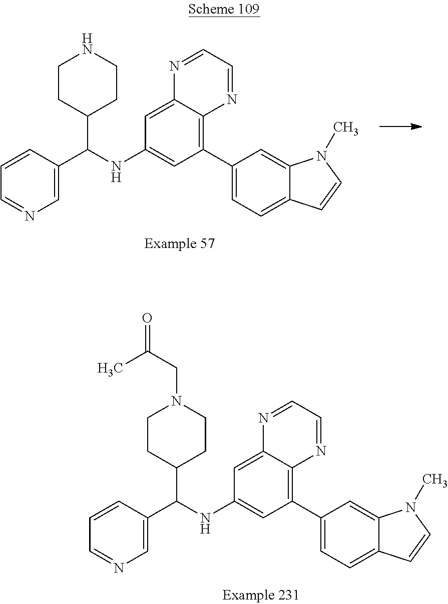 [SMILES: Cn1ccc2ccc(-c3cc(NC(c4cccnc4)C4CCNCC4)cc4nccnc34)cc21 ; CC(=O)CN1CCC(C(Nc2cc(-c3ccc4ccn(C)c4c3)c3nccnc3c2)c2cccnc2)CC1]

The product is prepared according to General Procedure 35, described in Example 82 with [8-(1-methyl-1H-indol-6-yl)-quinoxalin-6-yl]-(piperidin-4-yl-pyridin-3-yl-methyl)-amine (Example 57) (60.00 mg; 0.13 mmol; 1.00 eq.), 1-chloro-propan-2-one (12.12 μL; 0.15 mmol; 1.10 eq.), TEA (43.38 μL; 0.33 mmol; 2.50 eq.) and DCM anhydrous (5 mL). Purification by FCC (DCM:MeOH; gradient). 1-(4-{[8-(1-Methyl-1H-indol-6-yl)-quinoxalin-6-ylamino]-pyridin-3-yl-methyl}-piperidin-1-yl)-propan-2-one (30.00 mg; yield 42.2%) is obtained as a yellow powder.

### Example 232

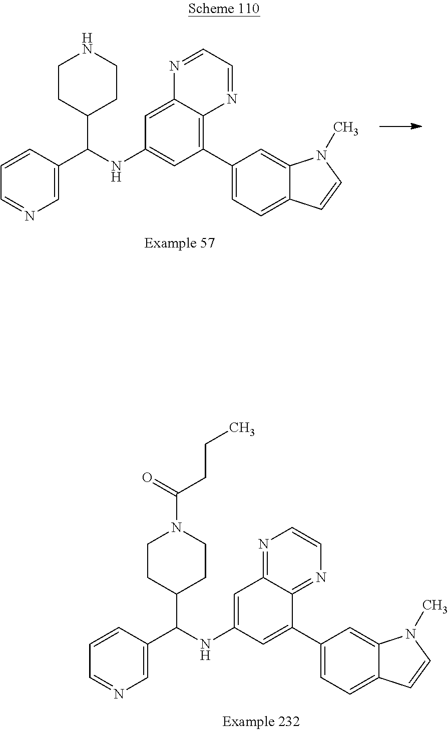 [SMILES: Cn1ccc2ccc(-c3cc(NC(c4cccnc4)C4CCNCC4)cc4nccnc34)cc21 ; CCCC(=O)N1CCC(C(Nc2cc(-c3ccc4ccn(C)c4c3)c3nccnc3c2)c2cccnc2)CC1]

The product is prepared according to General Procedure 35, described in Example 82 with [8-(1-methyl-1H-indol-6-yl)-quinoxalin-6-yl]-(piperidin-4-yl-pyridin-3-yl-methyl)-amine (Example 57) (60.00 mg; 0.13 mmol; 1.00 eq.), TEA (43.38 μL; 0.33 mmol; 2.50 eq.), butyryl chloride (15.28 μL; 0.15 mmol; 1.10 eq.) and DCM anhydrous (5 mL). Purification by FCC (DCM:MeOH; gradient). 1-(4-{[8-(1-Methyl-1H-indol-6-yl)-quinoxalin-6-ylamino]-pyridin-3-yl-methyl}-piperidin-1-yl)-butan-1-one (35.00 mg; yield 48.3%) is obtained as a yellow powder.

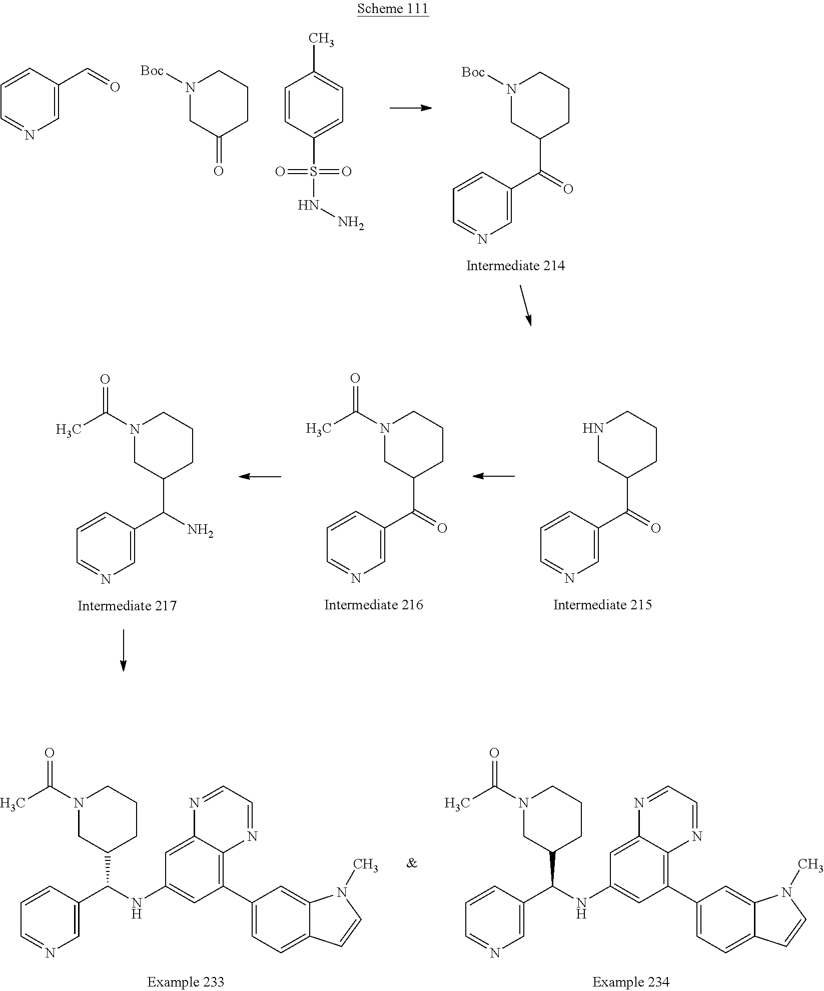

### Intermediate 214

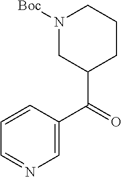 [SMILES: CC(C)(C)OC(=O)N1CCCC(C(=O)c2cccnc2)C1]

The product is prepared according to General Procedure 12, described for Intermediate 10 with 3-oxo-piperidine-1-carboxylic acid tert-butyl ester (0.91 mL; 5.02 mmol; 1.00 eq.), pyridine-3-carbaldehyde (0.47 mL; 5.02 mmol; 1.00 eq.), 4-methylbenzenesulfonohydrazide (0.93 g; 5.02 mmol; 1.00 eq.), Cs₂CO₃ (1.23 g; 3.76 mmol; 0.75 eq.), 1,4-dioxane (10 mL) and MeOH (10 mL). Purification by FCC (hexane/EtOAc; gradient; silica gel washed with 1% Et₃N/DCM and DCM). 3-(Pyridine-3-carbonyl)-piperidine-1-carboxylic acid tert-butyl ester (265.00 mg; yield 18.2%) is obtained as a yellow solid.

### Intermediate 215

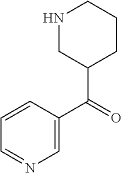 [SMILES: O=C(c1cccnc1)C1CCCNC1]

The product is prepared according to General Procedure 10, described in Example 44 with 3-(pyridine-3-carbonyl)-piperidine-1-carboxylic acid tert-butyl ester (Intermediate 214) (250.00 mg; 0.80 mmol; 1.00 eq.) and TFA (2 mL). Purification by FCC (puriflash NH2 20G; hexane/EtOAc; gradient). Piperidin-3-yl-pyridin-3-yl-methanone (300.00 mg; yield 181.2%) is obtained as a yellow gum.

### Intermediate 216

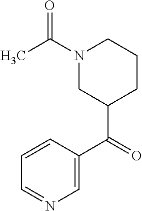 [SMILES: CC(=O)N1CCCC(C(=O)c2cccnc2)C1]

The product is prepared according to General Procedure 16, described for Intermediate 17 with piperidin-3-yl-pyridin-3-yl-methanone (Intermediate 215) (300.00 mg; 1.45 mmol; 1.00 eq.), acetic anhydride (0.15 mL; 1.60 mmol; 1.10 eq.), TEA (0.47 ml; 3.63 mmol; 2.50 eq.) and DCM anhydrous (3.00 mL). Crude 1-[3-(pyridine-3-carbonyl)-piperidin-1-yl]-ethanone (83.00 mg; yield 22.7%; yellow oil) is used for the next reaction without further purification.

### Intermediate 217

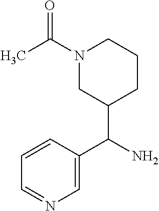 [SMILES: CC(=O)N1CCCC(C(N)c2cccnc2)C1]

The product is prepared according to General Procedure 14, described for Intermediate 12 with 1-[3-(pyridine-3-carbonyl)-piperidin-1-yl]-ethanone (Intermediate 216) (83.00 mg; 0.317 mmol; 1.0 eq.), 7M NH₃ in MeOH (3.50 mL), TTIP (0.19 mL; 0.63 mmol; 2.00 eq.) and NaBH₄ (47.95 mg; 1.27 mmol; 4.00 eq.). Crude 1-[3-(amino-pyridin-3-yl-methyl)-piperidin-1-yl]-ethanone (100.00 mg; yield 116.3%) is directly used in the next step without further purification.

### Example 233 & Example 234

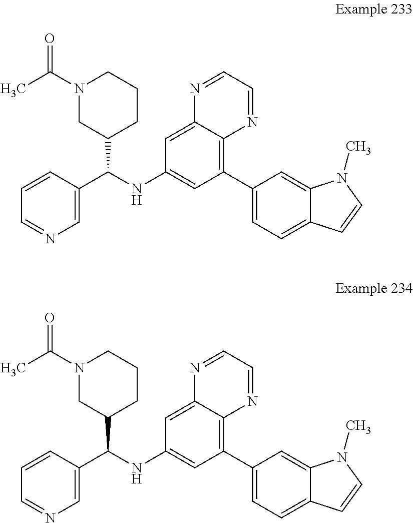 [SMILES: CC(=O)N1CCCC([C@H](Nc2cc(-c3ccc4ccn(C)c4c3)c3nccnc3c2)c2cccnc2)C1 ; CC(=O)N1CCCC([C@@H](Nc2cc(-c3ccc4ccn(C)c4c3)c3nccnc3c2)c2cccnc2)C1]

The products are prepared according to General Procedure 2, described in Example 1 with 7-chloro-5-(1-methyl-1H-indol-6-yl)-quinoxaline (60.00 mg; 0.19 mmol; 1.00 eq.), 1-[3-(amino-pyridin-3-yl-methyl)-piperidin-1-yl]-ethanone (Intermediate 217) (90.77 mg; 0.33 mmol; 1.80 eq.), NaOtBu (53.53 mg; 0.56 mmol; 3.00 eq.), BINAP (11.57 mg; 0.02 mmol; 0.10 eq.), Pd₂(dba)₃ (8.51 mg; 0.01 mmol; 0.05 eq.) and toluene (4 mL). Purification by FCC (Puriflash CN 50 uM 2×20G; hexane/EtOAc; gradient, next EtOAc/MeOH; gradient). Two diastereomers: 1-((R)-3-{(R)-[8-(1-methyl-1H-indol-6-yl)-quinoxalin-6-ylamino]-pyridin-3-yl-methyl}-piperidin-1-yl)-ethanone (24.50 mg; yield 23.7%; 88% by HPLC; yellow powder) and 1-((S)-3-{(R)-[8-(1-methyl-1H-indol-6-yl)-quinoxalin-6-ylamino]-pyridin-3-yl-methyl}-piperidin-1-yl)-ethanone (28.00 mg; yield 27.7%; 90.1% by HPLC; yellow powder) are obtained.

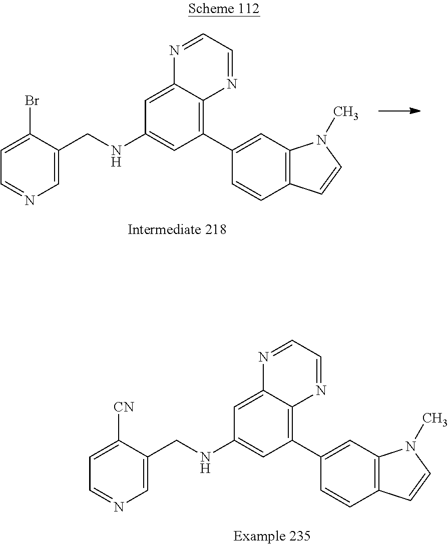 [SMILES: Cn1ccc2ccc(-c3cc(NCc4cnccc4Br)cc4nccnc34)cc21 ; Cn1ccc2ccc(-c3cc(NCc4cnccc4C#N)cc4nccnc34)cc21]

### Intermediate 218

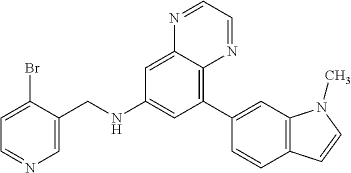 [SMILES: Cn1ccc2ccc(-c3cc(NCc4cnccc4Br)cc4nccnc34)cc21]

The product is prepared according to General Procedure 23, described in Example 63 with 4-bromo-pyridine-3-carbaldehyde hydrobromide (0.63 mL; 2.11 mmol; 1.50 eq.), 8-(1-methyl-1H-indol-6-yl)-quinoxalin-6-ylamine (Intermediate 22) (400.00 mg; 1.41 mmol; 1.00 eq.), Hantzsch ester (535.17 mg; 2.11 mmol; 1.50 eq.), TMCS (0.05 mL; 0.42 mmol; 0.30 eq.) and DCM anhydrous (5 mL). Purification by FCC (hexane/EtOAc; gradient; next EtOAc/MeOH; gradient). (4-Bromo-pyridin-3-ylmethyl)-[8-(1-methyl-1H-indol-6-yl)-quinoxalin-6-yl]-amine (257.90 mg; yield 41.0%) is obtained as a bright yellow foam.

### Example 235—General Procedure 63

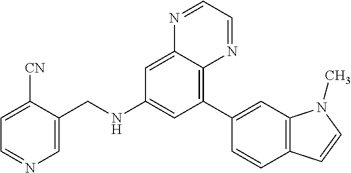 [SMILES: Cn1ccc2ccc(-c3cc(NCc4cnccc4C#N)cc4nccnc34)cc21]

To a glass tube (4-bromo-pyridin-3-ylmethyl)-[8-(1-methyl-1H-indol-6-yl)-quinoxalin-6-yl]-amine (Intermediate 218) (100.00 mg; 0.22 mmol; 1.00 eq.) and zinc cyanide (28.84 mg; 0.25 mmol; 1.10 eq.) are added followed by Pd(PPh₃)₄ (12.90 mg; 0.01 mmol; 0.05 eq.). RM is degassed and purged with argon thrice. Then DMA anhydrous (2.5 mL) is added and RM is stirred at 110° C. for 8h. Purification by FCC (hexane/EtOAc: gradient; next EtOAc/MeOH; gradient). 3-{[8-(1-Methyl-1H-indol-6-yl)-quinoxalin-6-ylamino]-methyl}-isonicotinonitrile (52.00 mg; yield 57.9%; 97% by HPLC) is obtained as a green powder.

### Example 236 & Example 237

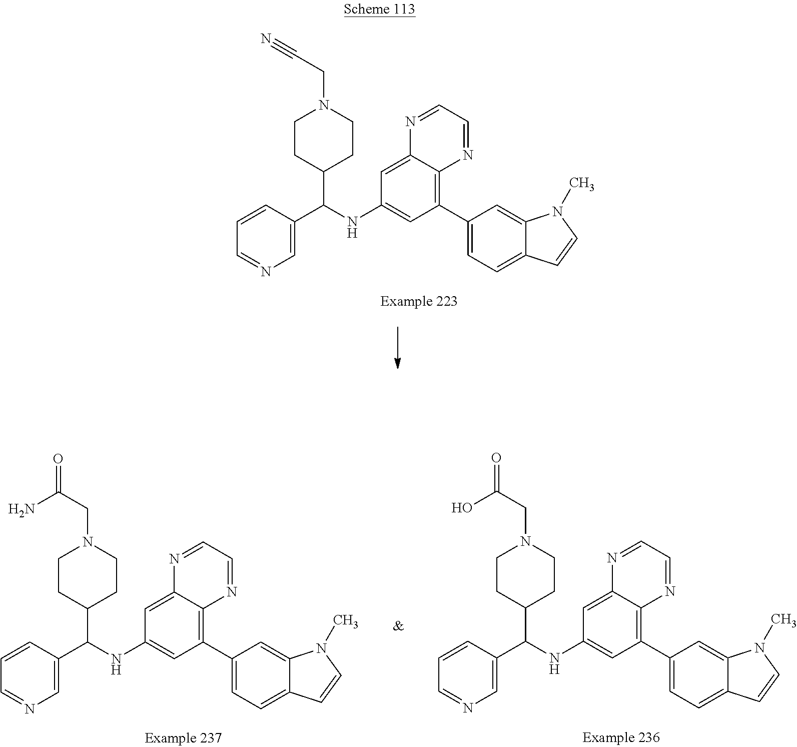 [SMILES: Cn1ccc2ccc(-c3cc(NC(c4cccnc4)C4CCN(CC#N)CC4)cc4nccnc34)cc21 ; Cn1ccc2ccc(-c3cc(NC(c4cccnc4)C4CCN(CC(N)=O)CC4)cc4nccnc34)cc21 ; Cn1ccc2ccc(-c3cc(NC(c4cccnc4)C4CCN(CC(=O)O)CC4)cc4nccnc34)cc21]

The products are prepared according to General Procedure 7, described in Example 35 with (4-{[8-(1-methyl-1H-indol-6-yl)-quinoxalin-6-ylamino]-pyridin-3-yl-methyl}-piperidin-1-yl)-acetonitrile (Example 223) (60.00 mg; 0.12 mmol; 1.00 eq.), KOH (20.71 mg; 0.37 mmol; 3.00 eq.) and t-BuOH (5 mL). Purification by FCC (DCM/MeOH; gradient). (4-{[8-(1-Methyl-1H-indol-6-yl)-quinoxalin-6-ylamino]-pyridin-3-yl-methyl}-piperidin-1-yl)-acetic acid (20.00 mg; yield 28.6%; 90.5% by HPLC) and 2-(4-{[8-(1-methyl-1H-indol-6-yl)-quinoxalin-6-ylamino]-pyridin-3-yl-methyl}-piperidin-1-yl)-acetamide (5.00 mg; yield 7.3%; 89% by HPLC) are obtained as yellow solids.

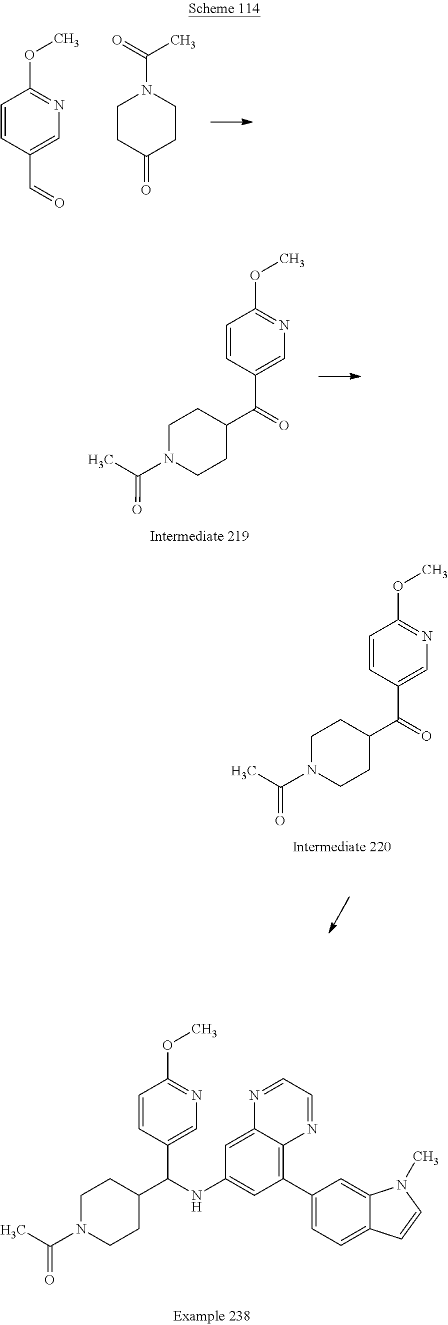

### Intermediate 219

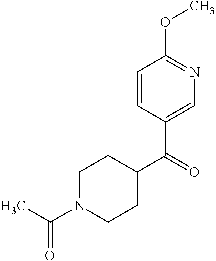 [SMILES: COc1ccc(C(=O)C2CCN(C(C)=O)CC2)cn1]

The product is prepared according to General Procedure 15, described for Intermediate 13 with 1-acetylpiperidin-4-one (500.00 mg; 3.54 mmol; 1.00 eq.), 6-methoxy-pyridine-3-carbaldehyde (0.43 mL; 3.54 mmol; 1.00 eq.), 4-methylbenzenesulfonohydrazide (659.62 mg; 3.54 mmol; 1.00 eq.), Cs₂CO₃ (2.89 g; 8.85 mmol; 2.50 eq.), MeOH (5 mL) and 1,4-dioxane (5 mL). Purification by FCC (DCM/MeOH; gradient). 1-[4-(6-Methoxy-pyridine-3-carbonyl)-piperidin-1-yl]-ethanone (278.00 mg; yield 28.6%) is obtained as a colorless oil.

### Intermediate 220

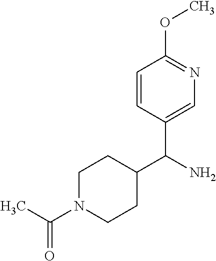 [SMILES: COc1ccc(C(N)C2CCN(C(C)=O)CC2)cn1]

The product is prepared according to General Procedure 14, described for Intermediate 12 with 1-[4-(6-methoxy-pyridine-3-carbonyl)-piperidin-1-yl]-ethanone (Intermediate 219) (278.00 mg; 1.01 mmol; 1.00 eq.), 7M NH₃ in MeOH (10 mL), TTIP (0.60 mL; 2.03 mmol; 2.00 eq.) and NaBH₄ (153.49 mg; 4.06 mmol; 4.00 eq.). Purification by FCC (EtOAc/MeOH; gradient). 1-{4-[Amino-(6-methoxy-pyridin-3-yl)-methyl]-piperidin-1-yl}-ethanone (96.90 mg; yield 24.7%) is obtained as a light brown oil.

### Example 238

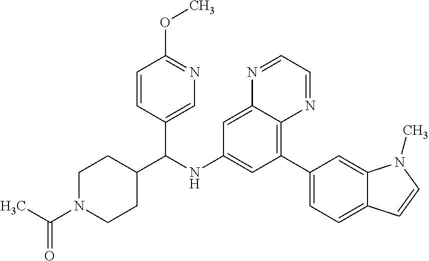 [SMILES: COc1ccc(C(Nc2cc(-c3ccc4ccn(C)c4c3)c3nccnc3c2)C2CCN(C(C)=O)CC2)cn1]

The product is prepared according to General Procedure 2, described in Example 1 with 7-chloro-5-(1-methyl-1H-indol-6-yl)-quinoxaline (Intermediate 4) (50.00 mg; 0.17 mmol; 1.00 eq.), 1-{4-[amino-(6-methoxy-pyridin-3-yl)-methyl]-piperidin-1-yl}-ethanone (Intermediate 220) (96.90 mg; 0.25 mmol; 1.50 eq.), NaOtBu (56.11 mg; 0.58 mmol; 3.50 eq.), BINAP (20.77 mg; 0.03 mmol; 0.20 eq.), Pd₂(dba)₃ (15.27 mg; 0.02 mmol; 0.10 eq.) and toluene (5 mL). Purification by FCC (EtOAc/MeOH; gradient). 1-(4-{(6-Methoxy-pyridin-3-yl)-[8-(1-methyl-1H-indol-6-yl)-quinoxalin-6-ylamino]-methyl}-piperidin-1-yl)-ethanone (61.00 mg; yield 65.7%; 93% by HPLC) is obtained as a yellow amorphous powder.

### Example 239

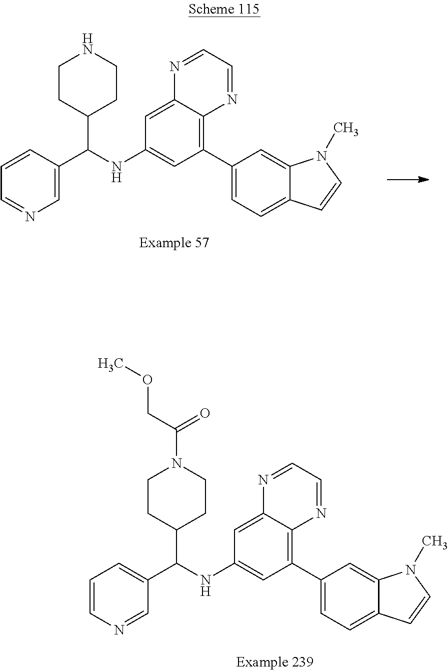 [SMILES: Cn1ccc2ccc(-c3cc(NC(c4cccnc4)C4CCNCC4)cc4nccnc34)cc21 ; COCC(=O)N1CCC(C(Nc2cc(-c3ccc4ccn(C)c4c3)c3nccnc3c2)c2cccnc2)CC1]

The product is prepared according to General Procedure 35, described in Example 82 with [8-(1-methyl-1H-indol-6-yl)-quinoxalin-6-yl]-(piperidin-4-yl-pyridin-3-yl-methyl)-amine (Example 57) (70.00 mg; 0.16 mmol; 1.00 eq.), methoxy-acetyl chloride (14.51 μL; 0.16 mmol; 1.00 eq.), TEA (50.61 μL; 0.39 mmol; 2.50 eq.) and DCM (2 mL). Purification by FCC (DCM/MeOH; gradient). 2-Methoxy-1-(4-{[8-(1-methyl-1H-indol-6-yl)-quinoxalin-6-ylamino]-pyridin-3-yl-methyl}-piperidin-1-yl)-ethanone (30.00 mg; yield 36.9%; 95% by HPLC) is obtained as a yellow powder.

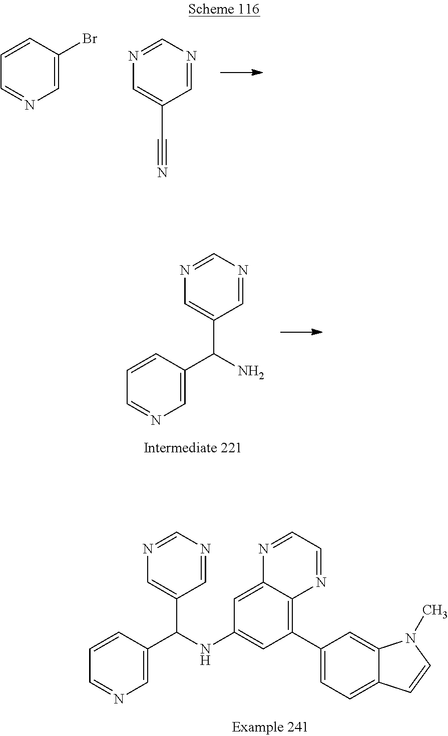 [SMILES: Brc1cccnc1 ; N#Cc1cncnc1 ; NC(c1cccnc1)c1cncnc1 ; Cn1ccc2ccc(-c3cc(NC(c4cccnc4)c4cncnc4)cc4nccnc34)cc21]

### Intermediate 221—General Procedure 64

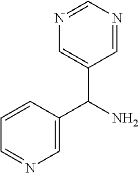 [SMILES: NC(c1cccnc1)c1cncnc1]

3-Bromo-pyridine (0.92 ml; 9.51 mmol; 2.00 eq.) and anhydrous THF (8 mL) are added in a dry flask. The solution is purged with nitrogen for 30 min. Isopropylmagnesium chloride/LiCl solution 1.3 M in THF (7.32 mL; 9.51 mmol; 2.00 eq.) is added dropwise to the reaction flask over a period of 10 min, and the mixture is stirred for 4 h at RT. The resulting solution of 3-pyridylmagnesium bromide is treated with solid pyrimidine-5-carbonitrile (0.50 g; 4.76 mmol; 1.00 eq.) at RT and to the resulting mixture NaBH₄ (0.72 g; 19.03 mmol; 4.00 eq.) is added, followed by water (0.2 mL, after 30 minutes). The mixture is stirred overnight. Next portion of water (10 mL) is added and the mixture concentrated in vacuo. The residue is dissolved in EtOAc and filtered. Oily residue is purified by FCC (DCM/MeOH; gradient) provided C-pyridin-3-yl-C-pyrimidin-5-yl-methylamine (100.00 mg; yield 11.3%; yellow oil).

### Example 241

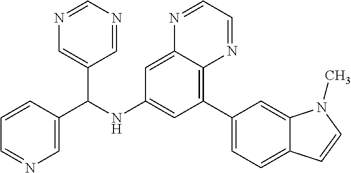 [SMILES: Cn1ccc2ccc(-c3cc(NC(c4cccnc4)c4cncnc4)cc4nccnc34)cc21]

The product is prepared according to General Procedure 6, described for Intermediate 6 with 7-chloro-5-(1-methyl-1H-indol-6-yl)-quinoxaline (Intermediate 4) (161.79 mg; 0.54 mmol; 1.00 eq.), C-pyridin-3-yl-C-pyrimidin-5-yl-methylamine (100.00 mg; 0.54 mmol; 1.00 eq.), Cs₂CO₃ (530.22 mg; 1.61 mmol; 3.00 eq.), BINAP (34.12 mg; 0.05 mmol; 0.10 eq.), Pd(OAc)₂ (12.69 mg; 0.05 mmol; 0.10 eq.) and 1,4-dioxane (3 mL). Purification by FCC (DCM/MeOH; gradient). [8-(1-Methyl-1H-indol-6-yl)-quinoxalin-6-yl]-(pyridin-3-yl-pyrimidin-5-yl-methyl)-amine (85.00 mg; yield 35.0%; 98% by HPLC) is obtained as a beige solid.

### Example 242

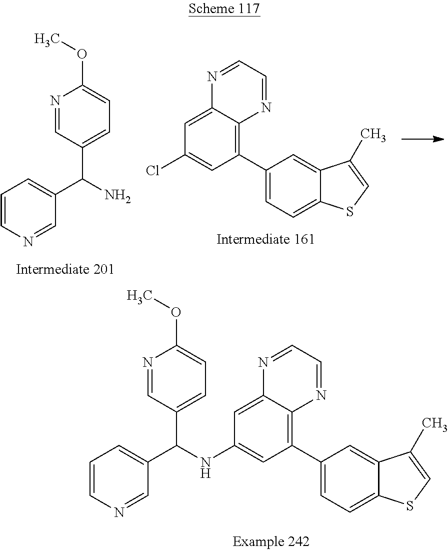 [SMILES: COc1ccc(C(N)c2cccnc2)cn1 ; Cc1csc2ccc(-c3cc(Cl)cc4nccnc34)cc12 ; COc1ccc(C(Nc2cc(-c3ccc4scc(C)c4c3)c3nccnc3c2)c2cccnc2)cn1]

The product is prepared according to General Procedure 6, described for Intermediate 6 with C-(6-methoxy-pyridin-3-yl)-C-pyridin-3-yl-methylamine (Intermediate 201) (26.00 mg; 0.10 mmol; 1.00 eq.), 7-chloro-5-(3-methyl-benzo[b]thiophen-5-yl)-quinoxaline (Intermediate 161) (36.28 mg; 0.10 mmol; 1.00 eq.), Cs₂CO₃ (102.56 mg; 0.31 mmol; 3.00 eq.), BINAP (6.60 mg; 0.01 mmol; 0.10 eq.), Pd(OAc)₂ (2.45 mg; 0.01 mmol; 0.10 eq.) and 1,4-dioxane (2 mL). Purification by preparative HPLC (TFA acidic conditions). [(6-Methoxy-pyridin-3-yl)-pyridin-3-yl-methyl]-[8-(3-methyl-benzo[b]thiophen-5-yl)-quinoxalin-6-yl]-amine (18.00 mg; yield 34.7%; 98% by HPLC) is obtained as an orange solid.

### Example 243

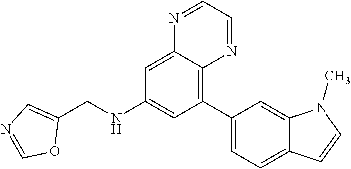 [SMILES: Cn1ccc2ccc(-c3cc(NCc4cnco4)cc4nccnc34)cc21]

The product is prepared according to General Procedure 23, described in Example 63 with 8-(1-methyl-1H-indol-6-yl)-quinoxalin-6-ylamine (Intermediate 22) (100.00 mg; 0.35 mmol; 1.00 eq.), oxazole-5-carbaldehyde (33.97 mg; 0.35 mmol; 1.00 eq.), Hantzsch ester (110.80 mg; 0.44 mmol; 1.25 eq.), TMCS (8.88 μL; 0.07 mmol; 0.20 eq.) and DCM anhydrous (3 mL). Purification by FCC (hexane/EtOAc: gradient and repurification by preparative HPLC (acidic conditions). [8-(1-Methyl-1H-indol-6-yl)-quinoxalin-6-yl]-oxazol-5-ylmethyl-amine (30.40 mg; yield 23.0%; 92% by HPLC) is obtained as a red powder; Purified Product).

### Example 244

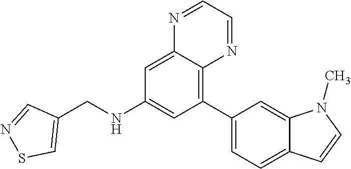 [SMILES: Cn1ccc2ccc(-c3cc(NCc4cnsc4)cc4nccnc34)cc21]

The product is prepared according to General Procedure 23, described in Example 63 with 8-(1-methyl-1H-indol-6-yl)-quinoxalin-6-ylamine (Intermediate 22) (100.00 mg; 0.35 mmol; 1.00 eq.), isothiazole-4-carbaldehyde (39.59 mg; 0.35 mmol; 1.00 eq.), Hantzsch ester (110.80 mg; 0.44 mmol; 1.25 eq.), TMCS (8.88 μL; 0.07 mmol; 0.20 eq.) and DCM anhydrous (3 mL). Purification by FCC (hexane/EtOAc: gradient and repurification by preparative HPLC (acidic conditions). Isothiazol-4-ylmethyl-[8-(1-methyl-1H-indol-6-yl)-quinoxalin-6-yl]-amine (79.20 mg; yield 60.4%; 99% by HPLC) is obtained as a red powder.

### Example 245

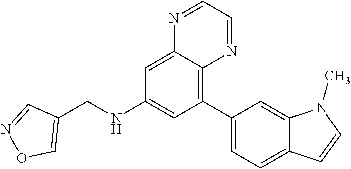 [SMILES: Cn1ccc2ccc(-c3cc(NCc4cnoc4)cc4nccnc34)cc21]

The product is prepared according to General Procedure 23, described in Example 63 with 8-(1-methyl-1H-indol-6-yl)-quinoxalin-6-ylamine (Intermediate 22) (100.00 mg; 0.35 mmol; 1.00 eq.), isoxazole-4-carbaldehyde (33.97 mg; 0.35 mmol; 1.00 eq.), Hantzsch ester (110.80 mg; 0.44 mmol; 1.25 eq.), TMCS (8.88 μL; 0.07 mmol; 0.20 eq.) and DCM anhydrous (3 mL). Purification by FCC (hexane/EtOAc: gradient and repurification by preparative HPLC (acidic conditions). Isoxazol-4-ylmethyl-[8-(1-methyl-1H-indol-6-yl)-quinoxalin-6-yl]-amine (64.40 mg; yield 48.8%; 94% by HPLC) is obtained as a red powder.

### Example 246

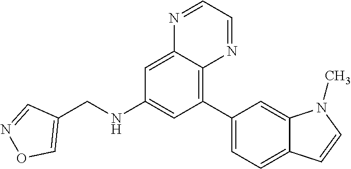 [SMILES: Cn1ccc2ccc(-c3cc(NCc4cnoc4)cc4nccnc34)cc21]

The product is prepared according to General Procedure 23, described in Example 63 with 8-(1-methyl-1H-indol-6-yl)-quinoxalin-6-ylamine (Intermediate 22) (100.00 mg; 0.35 mmol; 1.00 eq.), thiazole-5-carbaldehyde (39.59 mg; 0.35 mmol; 1.00 eq.), Hantzsch ester (110.80 mg; 0.44 mmol; 1.25 eq.), TMCS (8.88 μL; 0.07 mmol; 0.20 eq.) and DCM anhydrous (3 mL). Purification by FCC (DCM/MeOH; gradient). [8-(1-Methyl-1H-indol-6-yl)-quinoxalin-6-yl]-thiazol-5-ylmethyl-amine (86.50 mg; yield 61.4%; 92% by HPLC) is obtained as a yellow powder.

### Example 247

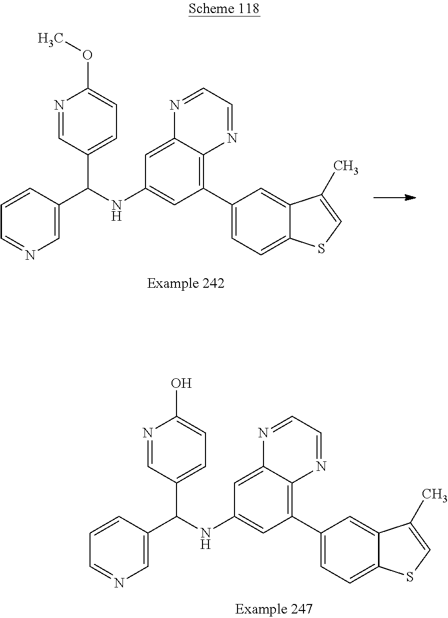 [SMILES: COc1ccc(C(Nc2cc(-c3ccc4scc(C)c4c3)c3nccnc3c2)c2cccnc2)cn1 ; Cc1csc2ccc(-c3cc(NC(c4cccnc4)c4ccc(O)nc4)cc4nccnc34)cc12]

The suspension of [(6-methoxy-pyridin-3-yl)-pyridin-3-yl-methyl]-[8-(3-methyl-benzo[b]thiophen-5-yl)-quinoxalin-6-yl]-amine (Example 242) (40.00 mg; 0.07 mmol; 1.00 eq.) and methylsulfanylsodium (14.60 mg; 0.21 mmol; 3.00 eq.) in DMF (2 mL) is stirred for 48 h at 60° C. TFA (0.5 mL) is added, and the mixture is evaporated to dryness. The yellow oily residue is purified by preparative HPLC (acidic conditions). 5-{[8-(3-Methyl-benzo[b]thiophen-5-yl)-quinoxalin-6-ylamino]-pyridin-3-yl-methyl}-pyridin-2-ol (14.00 mg; yield 42.0%; 99% by HPLC) is obtained as an orange solid.

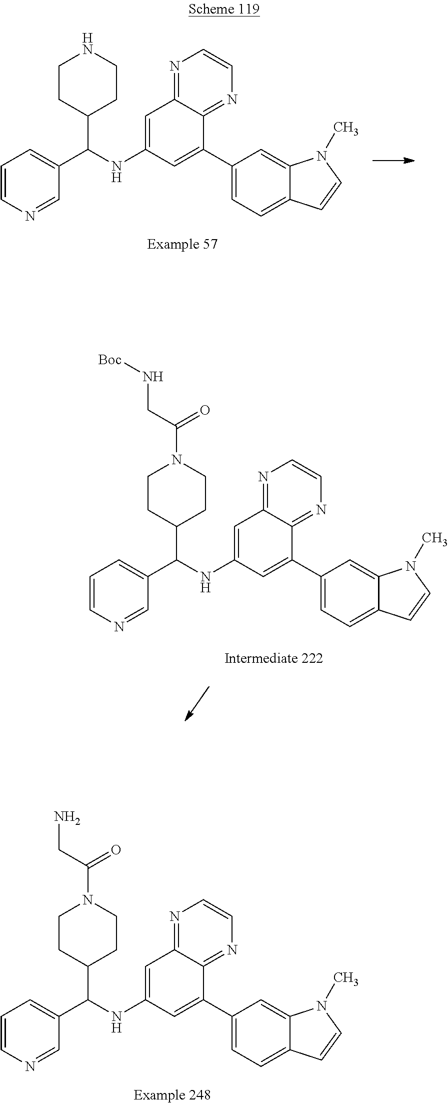 [SMILES: Cn1ccc2ccc(-c3cc(NC(c4cccnc4)C4CCNCC4)cc4nccnc34)cc21 ; Cn1ccc2ccc(-c3cc(NC(c4cccnc4)C4CCN(C(=O)CNC(=O)OC(C)(C)C)CC4)cc4nccnc34)cc21 ; Cn1ccc2ccc(-c3cc(NC(c4cccnc4)C4CCN(C(=O)CN)CC4)cc4nccnc34)cc21]

### Intermediate 222—General Procedure 65

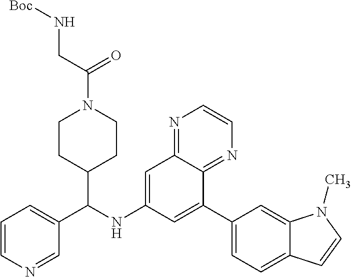 [SMILES: Cn1ccc2ccc(-c3cc(NC(c4cccnc4)C4CCN(C(=O)CNC(=O)OC(C)(C)C)CC4)cc4nccnc34)cc21]

[8-(1-Methyl-1H-indol-6-yl)-quinoxalin-6-yl]-(piperidin-4-yl-pyridin-3-yl-methyl)-amine (Example 57) (50.00 mg; 0.11 mmol; 1.00 eq.) and DCC (25.30 mg; 0.12 mmol; 1.10 eq.) are suspended in DCM anhydrous (3 mL). RM was stirred at 0° C. for 30 min, then tert-Butoxycarbonylamino-acetic acid (21.48 mg; 0.12 mmol; 1.10 eq.) is added at 0° C. The RM is gently warmed up to rt and then stirred at rt for 12h. Progress of reaction is monitored by TLC and UPLC. RM is partitioned between DCM and water. The aqueous layer is separated and extracted with 20% iPrOH/DCM. The combined organic layers are washed with brine and concentrated in vacuo. [2-(4-{[8-(1-Methyl-1H-indol-6-yl)-quinoxalin-6-ylamino]-pyridin-3-yl-methyl}-piperidin-1-yl)-2-oxo-ethyl]-carbamic acid tert-butyl ester (35.00 mg; yield 51.1%; 98% by UPLC) is obtained as a yellow powder.

### Example 248

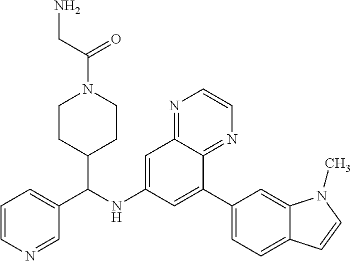 [SMILES: Cn1ccc2ccc(-c3cc(NC(c4cccnc4)C4CCN(C(=O)CN)CC4)cc4nccnc34)cc21]

The product is prepared according to General Procedure 11, described in Example 46 with [2-(4-{[8-(1-methyl-1H-indol-6-yl)-quinoxalin-6-ylamino]-pyridin-3-yl-methyl}-piperidin-1-yl)-2-oxo-ethyl]-carbamic acid tert-butyl ester (Intermediate 222) (35.00 mg; 0.06 mmol; 1.00 eq.), DCM (2 mL) and 2M HCl in Et₂O. Purification by FCC (CN 30 UM column; DCM/MeOH; gradient) and repurification by preparative HPLC (acidic conditions). 2-Amino-1-(4-{[8-(1-methyl-1H-indol-6-yl)-quinoxalin-6-ylamino]-pyridin-3-yl-methyl}-piperidin-1-yl)-ethanone (5.00 mg; yield 17.3%; 99% by HPLC) is obtained as a yellow-orange powder.

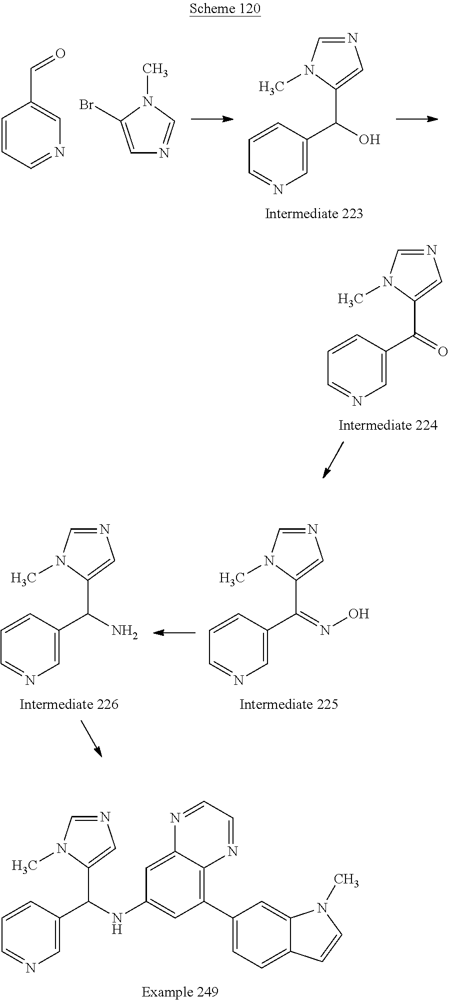

### Intermediate 223

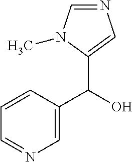

The product is prepared according to General Procedure 60, described for Intermediate 198 with 5-bromo-1-methyl-imidazole (450.94 mg; 2.80 mmol; 2.50 eq.), isopropylmagnesium chloride/LiCl solution 1.3 M in THF (2.15 mL; 2.80 mmol; 2.50 eq.), Pyridine-3-carbaldehyde (120 mg; 1.12 mmol; 1.0 eq.) and anhydrous THF (7 mL). Purification by FCC (DCM/MeOH; gradient). (3-Methyl-3H-imidazol-4-yl)-pyridin-3-yl-methanol (120 mg; yield 32%) is obtained as a brown solid.

### Intermediate 224

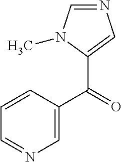

The product is prepared according to General Procedure 62, described for Intermediate 211 with (3-methyl-3H-imidazol-4-yl)-pyridin-3-yl-methanol (Intermediate 223) (150 mg, 0.8 mmol, 1.0 eq.), MnO₂ activated (0.38 g; 3.96 mmol; 5.00 eq.) and toluene. Purification by FCC (DCM/MeOH; gradient). (3-Methyl-3H-imidazol-4-yl)-pyridin-3-yl-methanone (70.00 mg; yield 47.2%) is obtained as a beige solid.

### Intermediate 225

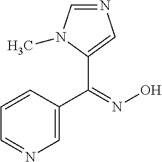

The product is prepared according to General Procedure 17, described for Intermediate 19 with (3-methyl-3H-imidazol-4-yl)-pyridin-3-yl-methanone (Intermediate 224) (90.00 mg; 0.47 mmol; 0.98 eq.), hydroxylamine hydrochloride (164 mg; 2.4 mmol; 5.0 eq.), sodium acetate (194 mg; 2.4 mmol; 5 eq.) and MeOH anhydrous (30 mL). Crude (3-methyl-3H-imidazol-4-yl)-pyridin-3-yl-methanone oxime (90.00 mg; yield 91.2%; light brown oil) is used in the next step.

### Intermediate 226

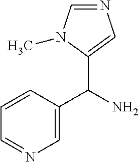

The product is prepared according to General Procedure 18, described for Intermediate 20 with (3-methyl-3H-imidazol-4-yl)-pyridin-3-yl-methanone oxime (Intermediate 225) (90.00 mg; 0.43 mmol; 1.00 eq.), zinc powder (141.15 mg; 2.16 mmol; 5.00 eq.), NH₄OAc (49.92 mg; 0.65 mmol; 1.50 eq.), ammonia 28% (3 mL), EtOH (3 mL) and water (3 mL). Crude C-(3-methyl-3H-imidazol-4-yl)-C-pyridin-3-yl-methylamine (120.00 mg; yield 141.5%; light yellow oil;) is used in to next step.

### Example 249

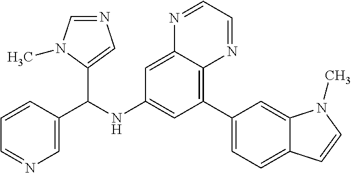 [SMILES: Cn1cncc1C(Nc1cc(-c2ccc3ccn(C)c3c2)c2nccnc2c1)c1cccnc1]

The product is prepared according to General Procedure 2, described in Example 1 with 7-chloro-5-(1-methyl-1H-indol-6-yl)-quinoxaline (Intermediate 4) (60.00 mg; 0.20 mmol; 1.00 eq.), C-(3-methyl-3H-imidazol-4-yl)-C-pyridin-3-yl-methylamine (Intermediate 226) (39.13 mg; 0.20 mmol; 1.00 eq.), NaOtBu (38.28 mg; 0.40 mmol; 2.00 eq.), BINAP (18.60 mg; 0.03 mmol; 0.15 eq.), Pd₂(dba)₃ (9.12 mg; 0.01 mmol; 0.05 eq.), toluene (4 mL) and 1,4-dioxane (1.00 mL). Purification by FCC (hexane/EtOAc; gradient and then EtOAc/MeOH; gradient). [(3-Methyl-3H-imidazol-4-yl)-pyridin-3-yl-methyl]-[8-(1-methyl-1H-indol-6-yl)-quinoxalin-6-yl]-amine (10.00 mg; yield 11.1%; 98% by HPLC) is obtained as a yellow powder.

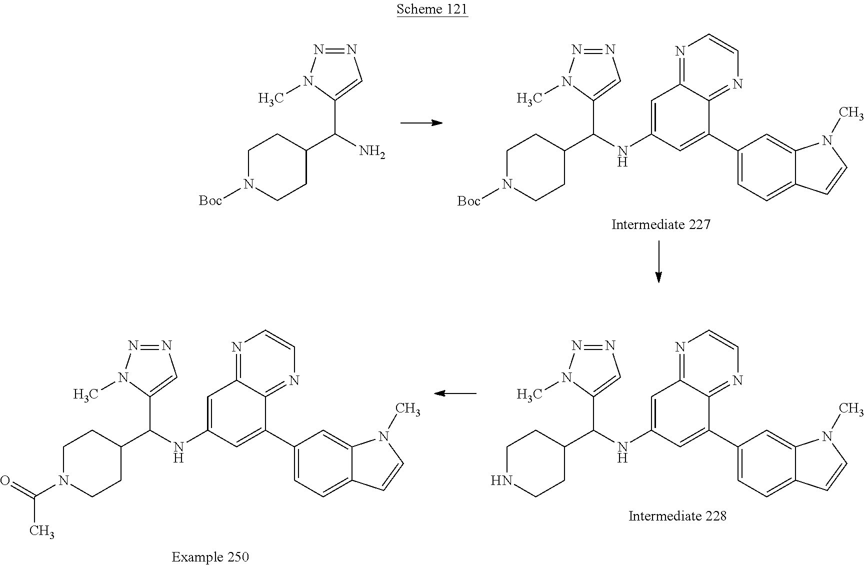 [SMILES: Cn1nncc1C(N)C1CCN(C(=O)OC(C)(C)C)CC1 ; Cn1nncc1C(Nc1cc(-c2ccc3ccn(C)c3c2)c2nccnc2c1)C1CCN(C(=O)OC(C)(C)C)CC1 ; CC(=O)N1CCC(C(Nc2cc(-c3ccc4ccn(C)c4c3)c3nccnc3c2)c2cnnn2C)CC1 ; Cn1nncc1C(Nc1cc(-c2ccc3ccn(C)c3c2)c2nccnc2c1)C1CCNCC1]

### Intermediate 227

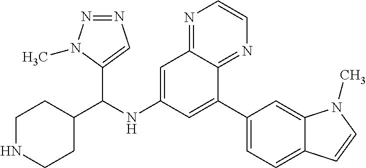 [SMILES: Cn1nncc1C(Nc1cc(-c2ccc3ccn(C)c3c2)c2nccnc2c1)C1CCNCC1]

The product is prepared according to General Procedure 2, described in Example 1 with 7-chloro-5-(1-methyl-1H-indol-6-yl)-quinoxaline (Intermediate 4) (80.00 mg; 0.27 mmol; 1.00 eq.), 4-[amino-(3-methyl-3H-[1,2,3]triazol-4-yl)-methyl]-piperidine-1-carboxylic acid tert-butyl ester (120.67 mg; 0.41 mmol; 1.50 eq.), NaOtBu (52.34 mg; 0.54 mmol; 2.00 eq.), Pd₂(dba)₃ (24.94 mg; 0.03 mmol; 0.10 eq.), BINAP (33.92 mg; 0.05 mmol; 0.20 eq.) and toluene (5 mL). Purification by FCC (DCM/MeOH; gradient). 4-[[8-(1-Methyl-1 H-indol-6-yl)-quinoxalin-6-ylamino]-(3-methyl-3H-[1,2,3]triazol-4-yl)-methyl]-piperidine-1-carboxylic acid tert-butyl ester (120.00 mg; yield 79.7%) is obtained as a yellow oil.

### Intermediate 228

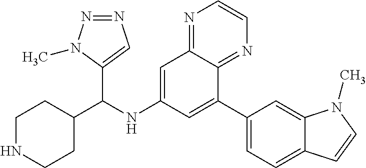 [SMILES: Cn1nncc1C(Nc1cc(-c2ccc3ccn(C)c3c2)c2nccnc2c1)C1CCNCC1]

The product is prepared according to General Procedure 10, described in Example 44 with 4-[[8-(1-methyl-1H-indol-6-yl)-quinoxalin-6-ylamino]-(3-methyl-3H-[1,2,3]triazol-4-yl)-methyl]-piperidine-1-carboxylic acid tert-butyl ester (Intermediate 227) (110.00 mg; 0.20 mmol; 1.00 eq.), TFA (1 mL) and DCM (3 mL). Crude [8-(1-methyl-1H-indol-6-yl)-quinoxalin-6-yl]-[(3-methyl-3H-[1,2,3]triazol-4-yl)-piperidin-4-yl-methyl]-amine (75.00 mg; yield 77.4%) is used in the next step without further purification.

### Example 250

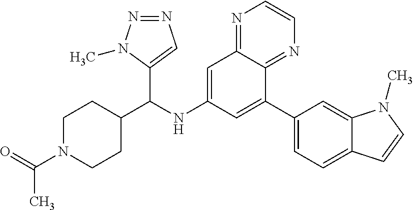 [SMILES: CC(=O)N1CCC(C(Nc2cc(-c3ccc4ccn(C)c4c3)c3nccnc3c2)c2cnnn2C)CC1]

The product is prepared according to General Procedure 16, described for Intermediate 17 with [8-(1-methyl-1H-indol-6-yl)-quinoxalin-6-yl]-[(3-methyl-3H-[1,2,3]triazol-4-yl)-piperidin-4-yl-methyl]-amine (Intermediate 228) (75.00 mg; 0.17 mmol; 1.00 eq.), acetic anhydride (17.23 μL; 0.18 mmol; 1.10 eq.), TEA (53.75 μL; 0.41 mmol; 2.50 eq.) and DCM anhydrous (10 mL). Purification by FCC (DCM/MeOH; gradient) and repurification by preparative HPLC (acidic conditions). 1-{4-[[8-(1-Methyl-1H-indol-6-yl)-quinoxalin-6-ylamino]-(3-methyl-3H-[1,2,3]triazol-4-yl)-methyl]-piperidin-1-yl}-ethanone (15.00 mg; yield 17.3%; 94% by HPLC) is obtained as a yellow powder.

### Example 251

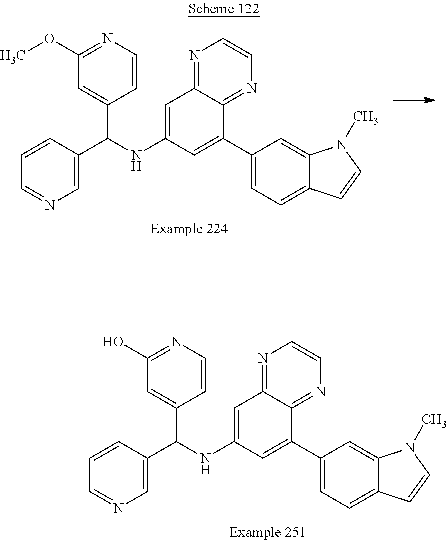 [SMILES: COc1cc(C(Nc2cc(-c3ccc4ccn(C)c4c3)c3nccnc3c2)c2cccnc2)ccn1 ; Cn1ccc2ccc(-c3cc(NC(c4cccnc4)c4ccnc(O)c4)cc4nccnc34)cc21]

The suspension of [(2-methoxy-pyridin-4-yl)-pyridin-3-yl-methyl]-[8-(1-methyl-1H-indol-6-yl)-quinoxalin-6-yl]-amine (Example 224) (290.00 mg; 0.61 mmol; 1.00 eq.) and methylsulfanylsodium (129.04 mg; 1.84 mmol; 3.00 eq.) in DMF (2 mL) is stirred for 48h at 60° C. TFA (0.5 mL) is added, and the mixture is evaporated to dryness. Purification by FCC (DCM/MeOH; gradient). 4-{[8-(1-Methyl-1H-indol-6-yl)-quinoxalin-6-ylamino]-pyridin-3-yl-methyl}-pyridin-2-ol (215.00 mg; yield 74.3%; 97% by HPLC) is obtained as a yellow powder.

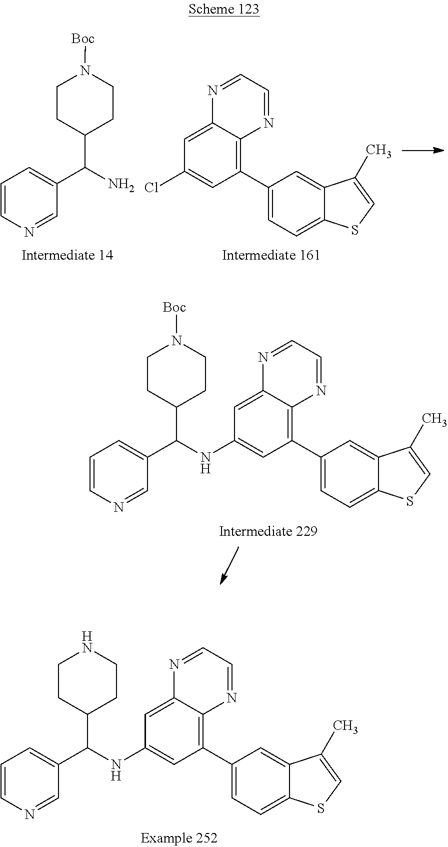

### Intermediate 229

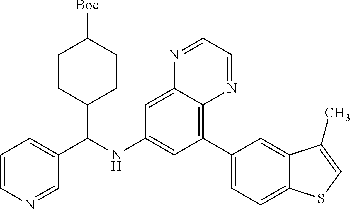 [SMILES: Cc1csc2ccc(-c3cc(NC(c4cccnc4)C4CCC(C(=O)OC(C)(C)C)CC4)cc4nccnc34)cc12]

The product is prepared according to General Procedure 2, described in Example 1 with 4-(amino-pyridin-3-yl-methyl)-piperidine-1-carboxylic acid tert-butyl eater (Intermediate 14) (126.57 mg; 0.43 mmol; 1.50 eq.), 7-chloro-5-(3-methyl-benzo[b]thiophen-5-yl)-quinoxaline (Intermediate 161) (90.00 mg; 0.29 mmol; 1.00 eq.) NaOtBu (55.66 mg; 0.58 mmol; 2.00 eq.), Pd₂(dba)₃ (26.52 mg; 0.03 mmol; 0.10 eq.), BINAP (36.06 mg; 0.06 mmol; 0.20 eq.), and toluene (5 mL). Purification by FCC (DCM/MeOH; gradient). 4-{[8-(3-Methyl-benzo[b]thiophen-5-yl)-quinoxalin-6-ylamino]-pyridin-3-yl-methyl}-piperidine-1-carboxylic acid tert-butyl ester (12.00 mg; yield 63.5%) is obtained as a yellow solid.

### Example 252

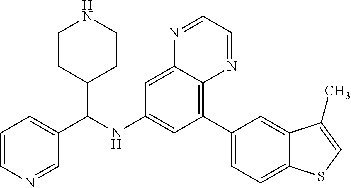 [SMILES: Cc1csc2ccc(-c3cc(NC(c4cccnc4)C4CCNCC4)cc4nccnc34)cc12]

The product is prepared according to General Procedure 10, described in Example 44 with 4-{[8-(3-methyl-benzo[b]thiophen-5-yl)-quinoxalin-6-ylamino]-pyridin-3-yl-methyl}-piperidine-1-carboxylic acid tert-butyl ester (Intermediate 229) (110.00 mg; 0.19 mmol; 1.00 eq.), TFA (1 mL) and DCM (3 mL). Purification by FCC (NH₂ column, DCM/MeOH; gradient). [8-(3-Methyl-benzo[b]thiophen-5-yl)-quinoxalin-6-yl]-(piperidin-4-yl-pyridin-3-yl-methyl)-amine (30.00 mg; yield 32.8%; 99% by HPLC) is obtained as a yellow powder.

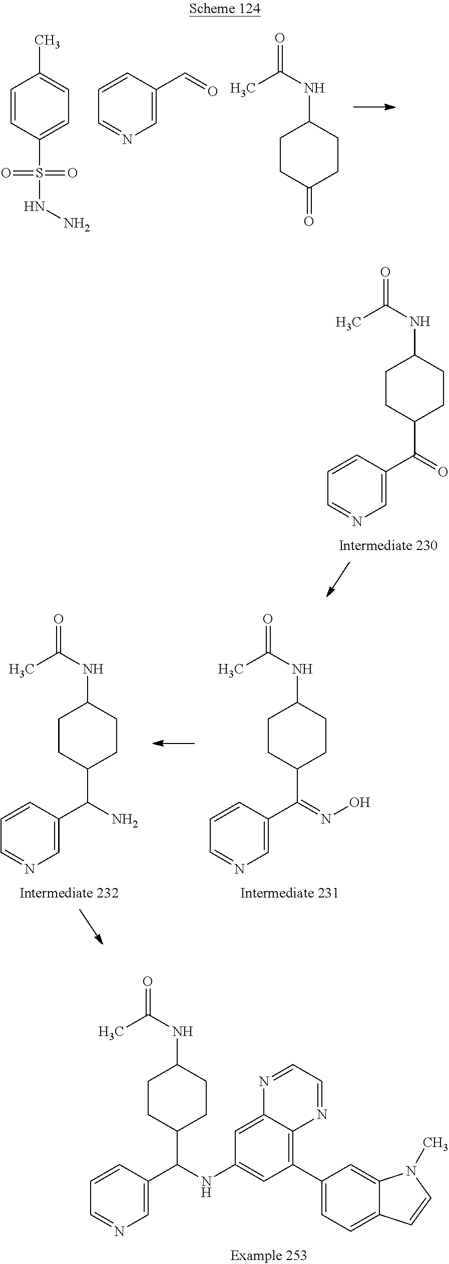

### Intermediate 230

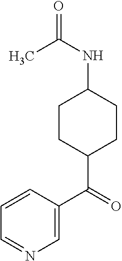 [SMILES: CC(=O)NC1CCC(C(=O)c2cccnc2)CC1]

The product is prepared according to General Procedure 15, described for Intermediate 13 with N-(4-oxo-cyclohexyl)-acetamide (2.00 g; 12.89 mmol; 1.00 eq.), 4-methylbenzenesulfonohydrazide (2.40 g; 12.89 mmol; 1.00 eq.), pyridine-3-carbaldehyde (1.21 mL; 12.89 mmol; 1.00 eq.), Cs₂CO₃ (10.50 g; 32.22 mmol; 2.50 eq.), MeOH (14 mL) and 1,4-dioxane (30 mL). Purification by FCC (DCM/MeOH; gradient). N-[4-(Pyridine-3-carbonyl)-cyclohexyl]-acetamide (1.35 g; yield 37.4%) is obtained as a beige solid.

### Intermediate 231

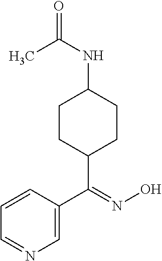 [SMILES: CC(=O)NC1CCC(/C(=N\O)c2cccnc2)CC1]

The product is prepared according to General Procedure 17, described for Intermediate 19 with N-[4-(pyridine-3-carbonyl)-cyclohexyl]-acetamide (Intermediate 230) (1.35 g; 5.48 mmol; 1.00 eq.), hydroxylamine hydrochloride (0.95 g; 13.70 mmol; 2.50 eq.), NaOAc (1.12 g; 13.70 mmol; 2.50 eq.) and MeOH anhydrous (30 mL). Crude N-(4-{[(E)-hydroxyimino]-pyridin-3-yl-methyl}-cyclohexyl)-acetamide (1.00 g; yield 67.0%; pale yellow gum) is used in the next step.

### Intermediate 232

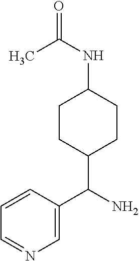 [SMILES: CC(=O)NC1CCC(C(N)c2cccnc2)CC1]

The product is prepared according to General Procedure 18, described for Intermediate 20 with N-(4-{[(E)-hydroxyimino]-pyridin-3-yl-methyl}-cyclohexyl)-acetamide (Intermediate 231) (1.00 g; 3.83 mmol; 1.00 eq.), NH₄OAc (442.45 mg; 5.74 mmol; 1.50 eq.), zinc dust (1.25 g; 19.13 mmol; 5.00 eq.), ammonia 25% (9.00 mL), EtOH (9 mL) and water (9 mL). Purification by FCC (DCM/MeOH; gradient). N-[4-(Amino-pyridin-3-yl-methyl)-cyclohexyl]-acetamide (378.00 mg; yield 65%) is obtained as a yellow oil.

### Example 253

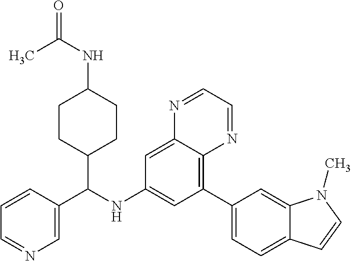 [SMILES: CC(=O)NC1CCC(C(Nc2cc(-c3ccc4ccn(C)c4c3)c3nccnc3c2)c2cccnc2)CC1]

The product is prepared according to General Procedure 2, described in Example 1 with 7-chloro-5-(1-methyl-1H-indol-6-yl)-quinoxaline (Intermediate 4) (100.00 mg; 0.31 mmol; 1.00 eq.), N-[4-(amino-pyridin-3-yl-methyl)-cyclohexyl]-acetamide (Intermediate 232) (252.05 mg; 0.62 mmol; 2.00 eq.), NaOtBu (119.48 mg; 1.24 mmol; 4.00 eq.), Bis(tri-tert-butylphosphine)palladium(0) (15.88 mg; 0.03 mmol; 0.10 eq.) and 1,4-dioxane (2 mL). Purification by FCC (hexane/EtOAc; gradient and next EtOAc/MeOH; gradient). N-(4-{[8-(1-Methyl-1H-indol-6-yl)-quinoxalin-6-ylamino]-pyridin-3-yl-methyl}-cyclohexyl)-acetamide (77.20 mg; yield 47.0%; 93% by HPLC) is obtained as a yellow powder.

### Example 254

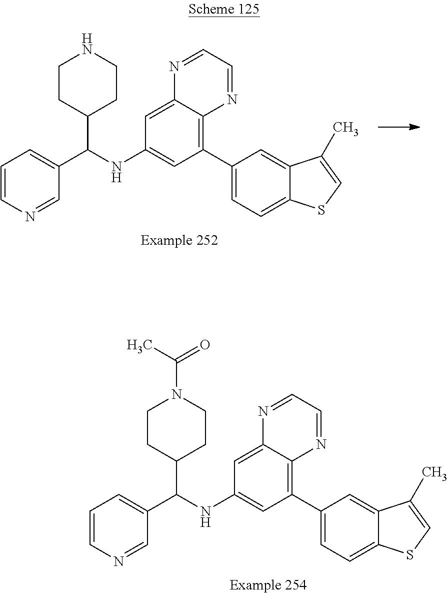 [SMILES: Cc1csc2ccc(-c3cc(NC(c4cccnc4)C4CCNCC4)cc4nccnc34)cc12 ; CC(=O)N1CCC(C(Nc2cc(-c3ccc4scc(C)c4c3)c3nccnc3c2)c2cccnc2)CC1]

The product is prepared according to General Procedure 16, described for Intermediate 17 with [8-(3-methyl-benzo[b]thiophen-5-yl)-quinoxalin-6-yl]-(piperidin-4-yl-pyridin-3-yl-methyl)-amine (Example 252) (40.00 mg; 0.09 mmol; 1.00 eq.), TEA (27.86 μL; 0.21 mmol; 2.50 eq.), acetic anhydride (8.93 μL; 0.09 mmol; 1.10 eq.) and DCM anhydrous (10 mL). Purification by FCC (DCM/MeOH; gradient). 1-(4-{[8-(3-Methyl-benzo[b]thiophen-5-yl)-quinoxalin-6-ylamino]-pyridin-3-yl-methyl}-piperidin-1-yl)-ethanone (15.00 mg; yield 30.7%; 89% by HPLC) is obtained as a light orange powder.

### Example 255 and Example 256

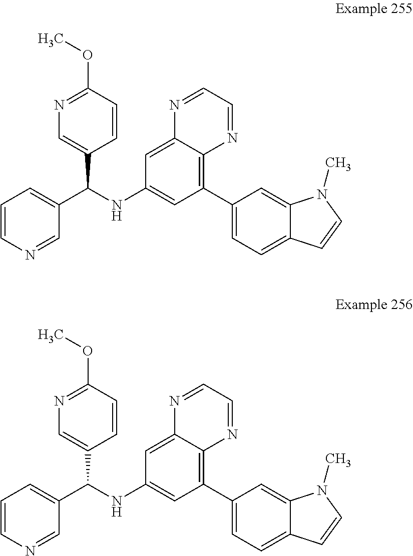 [SMILES: COc1ccc([C@@H](Nc2cc(-c3ccc4ccn(C)c4c3)c3nccnc3c2)c2cccnc2)cn1 ; COc1ccc([C@H](Nc2cc(-c3ccc4ccn(C)c4c3)c3nccnc3c2)c2cccnc2)cn1]

[(6-Methoxy-pyridin-3-yl)-pyridin-3-yl-methyl]-[8-(1-methyl-1H-indol-6-yl)-quinoxalin-6-yl]-amine (Example 242) (170.000 mg; 0.356 mmol; 1.0 eq.) is dissolved in isopropyl alcohol and compound is separated by HPLC (Chiralpak AD-H; 250×20 mm I.D., 5 uM). Both enantiomers: [(R)-(6-methoxy-pyridin-3-yl)-pyridin-3-yl-methyl]-[8-(1-methyl-1H-indol-6-yl)-quinoxalin-6-yl]-amine (Example 256) (70.00 mg; yield 41.6%; yellow solid) and [(S)-(6-methoxy-pyridin-3-yl)-pyridin-3-yl-methyl]-[8-(1-methyl-1H-indol-6-yl)-quinoxalin-6-yl]-amine (Example 255) (75.00 mg; yield 44.6%; yellow solid) are isolated with 99% of optical purity.

### Example 257

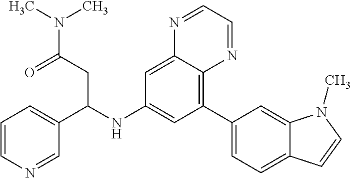 [SMILES: CN(C)C(=O)CC(Nc1cc(-c2ccc3ccn(C)c3c2)c2nccnc2c1)c1cccnc1]

The product is prepared according to General Procedure 6, described for Intermediate 6 with 7-chloro-5-(1-methyl-1H-indol-6-yl)-quinoxaline (Intermediate 4) (50.00 mg; 0.17 mmol; 1.00 eq.), 3-amino-N,N-dimethyl-3-pyridin-3-yl-propionamide (49.34 mg; 0.26 mmol; 1.50 eq.), Cs₂CO₃ (221.83 mg; 0.68 mmol; 4.00 eq.), BINAP (21.20 mg; 0.03 mmol; 0.20 eq.), Pd(OAc)₂ (5.73 mg; 0.03 mmol; 0.15 eq.) and 1,4-dioxane (2 mL). Purification by FCC (DCM/MeOH; gradient). N,N-Dimethyl-3-[8-(1-methyl-1H-indol-6-yl)-quinoxalin-6-ylamino]-3-pyridin-3-yl-propionamide (20.00 mg; yield 25.0%; 95% by HPLC) is obtained as a yellow solid.

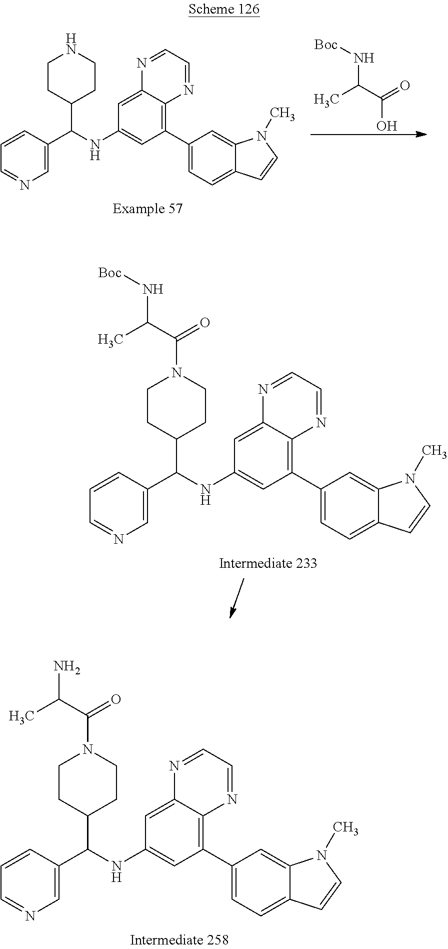 [SMILES: Cn1ccc2ccc(-c3cc(NC(c4cccnc4)C4CCNCC4)cc4nccnc34)cc21 ; CC(NC(=O)OC(C)(C)C)C(=O)O ; CC(NC(=O)OC(C)(C)C)C(=O)N1CCC(C(Nc2cc(-c3ccc4ccn(C)c4c3)c3nccnc3c2)c2cccnc2)CC1 ; CC(N)C(=O)N1CCC(C(Nc2cc(-c3ccc4ccn(C)c4c3)c3nccnc3c2)c2cccnc2)CC1]

### Intermediate 233

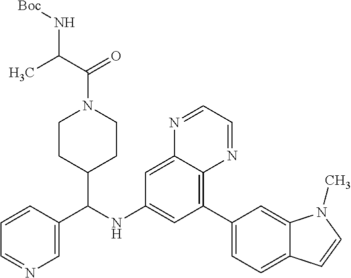 [SMILES: CC(NC(=O)OC(C)(C)C)C(=O)N1CCC(C(Nc2cc(-c3ccc4ccn(C)c4c3)c3nccnc3c2)c2cccnc2)CC1]

The product is prepared according to General Procedure 65, described for Intermediate 222 with 2-tert-butoxycarbonylamino-propionic acid (27.84 mg; 0.15 mmol; 1.10 eq.), [8-(1-methyl-1H-indol-6-yl)-quinoxalin-6-yl]-(piperidin-4-yl-pyridin-3-yl-methyl)-amine (Example 57) (60.00 mg; 0.13 mmol; 1.00 eq.), DCC (30.36 mg; 0.15 mmol; 1.10 eq.) and DCM anhydrous (4 mL). Crude [1-methyl-2-(4-{[8-(1-methyl-1H-indol-6-yl)-quinoxalin-6-ylamino]-pyridin-3-yl-methyl}-piperidin-1-yl)-2-oxo-ethyl]-carbamic acid tert-butyl ester (75.80 mg; yield 90.2%; yellow powder) is used in the next step.

### Example 258

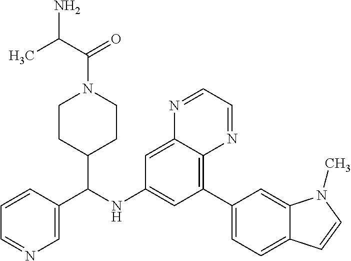 [SMILES: CC(N)C(=O)N1CCC(C(Nc2cc(-c3ccc4ccn(C)c4c3)c3nccnc3c2)c2cccnc2)CC1]

The product is prepared according to General Procedure 11, described in Example 46 with [1-methyl-2-(4-{[8-(1-methyl-1H-indol-6-yl)-quinoxalin-6-ylamino]-pyridin-3-yl-methyl}-piperidin-1-yl)-2-oxo-ethyl]-carbamic acid tert-butyl ester (intermediate 233) (75.80 mg; 0.12 mmol; 1.00 eq.), 2M HCl in Et₂O and DCM (3 mL). Purification by preparative HPLC (acidic conditions). 2-Amino-1-(4-{[8-(1-methyl-1H-indol-6-yl)-quinoxalin-6-ylamino]-pyridin-3-yl-methyl}-piperidin-1-yl)-propan-1-one formic acid (22.00 mg; yield 29.5%; 91% by HPLC) is obtained as a yellow powder.

### Example 259

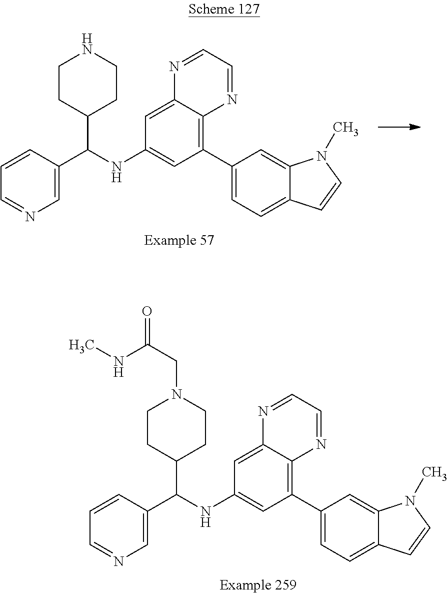 [SMILES: Cn1ccc2ccc(-c3cc(NC(c4cccnc4)C4CCNCC4)cc4nccnc34)cc21 ; CNC(=O)CN1CCC(C(Nc2cc(-c3ccc4ccn(C)c4c3)c3nccnc3c2)c2cccnc2)CC1]

The product is prepared according to General Procedure 35, described in Example 82 with [8-(1-methyl-1H-indol-6-yl)-quinoxalin-6-yl]-(piperidin-4-yl-pyridin-3-yl-methyl)-amine (Example 57) (50.00 mg; 0.11 mmol; 1.00 eq.), Cs₂CO₃ (54.48 mg; 0.17 mmol; 1.50 eq.), 2-chloro-N-methyl-acetamide (11.70 μl; 0.12 mmol; 1.10 eq.) and DCM anhydrous (10 mL). Purification by FCC (DCM/MeOH; gradient). N-Methyl-2-(4-{[8-(1-methyl-1H-indol-6-yl)-quinoxalin-6-ylamino]-pyridin-3-yl-methyl}-piperidin-1-yl)-acetamide (30.00 mg; yield 47.3%; 91% by HPLC) is obtained as a yellow powder.

### Example 260

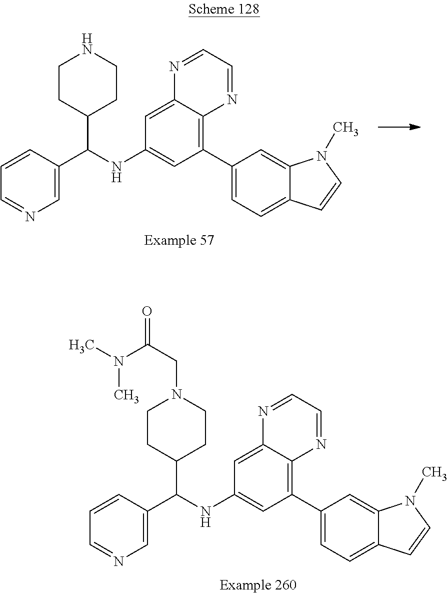 [SMILES: Cn1ccc2ccc(-c3cc(NC(c4cccnc4)C4CCNCC4)cc4nccnc34)cc21 ; CN(C)C(=O)CN1CCC(C(Nc2cc(-c3ccc4ccn(C)c4c3)c3nccnc3c2)c2cccnc2)CC1]

The product is prepared according to General Procedure 35, described in Example 82 with [8-(1-methyl-1H-indol-6-yl)-quinoxalin-6-yl]-(piperidin-4-yl-pyridin-3-yl-methyl)-amine (Example 57) (50.00 mg; 0.11 mmol; 1.00 eq.), Cs₂CO₃ (72.64 mg; 0.22 mmol; 2.00 eq.), 2-chloro-N,N-dimethylacetamide (27.10 mg; 0.22 mmol; 2.00 eq.) and DCM (5 mL). Purification by FCC (DCM/MeOH; gradient). N,N-Dimethyl-2-(4-{[8-(1-methyl-1H-indol-6-yl)-quinoxalin-6-ylamino]-pyridin-3-yl-methyl}-piperidin-1-yl)-acetamide (18.00 mg; yield 29.0%; 95% by HPLC) is obtained as a yellow powder.

### Example 261

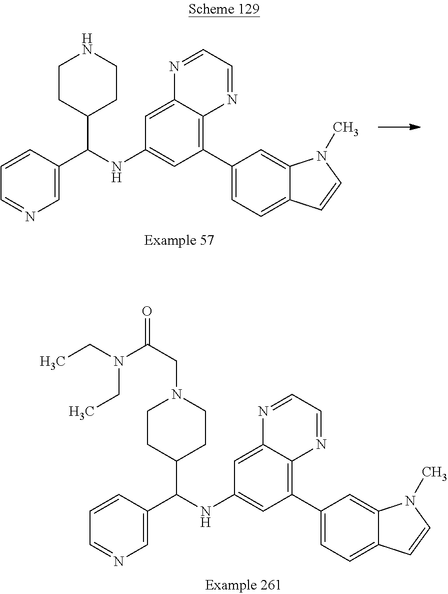 [SMILES: Cn1ccc2ccc(-c3cc(NC(c4cccnc4)C4CCNCC4)cc4nccnc34)cc21 ; CCN(CC)C(=O)CN1CCC(C(Nc2cc(-c3ccc4ccn(C)c4c3)c3nccnc3c2)c2cccnc2)CC1]

The product is prepared according to General Procedure 35, described in Example 82 with [8-(1-methyl-1H-indol-6-yl)-quinoxalin-6-yl]-(piperidin-4-yl-pyridin-3-yl-methyl)-amine (Example 57) (50.00 mg; 0.11 mmol; 1.00 eq.), 2-chloro-N,N-diethyl-acetamide (15.52 μL; 0.12 mmol; 1.10 eq.), TEA (36.15 μL; 0.28 mmol; 2.50 eq.) and DCM anhydrous (10 mL). Purification by FCC (DCM/MeOH; gradient) and repurification by preparative HPLC (acidic conditions). N,N-Diethyl-2-(4-{[8-(1-methyl-1H-indol-6-yl)-quinoxalin-6-ylamino]-pyridin-3-yl-methyl}-piperidin-1-yl)-acetamide (15.00 mg; yield 23.8%; 99% by HPLC) is obtained as a yellow powder.

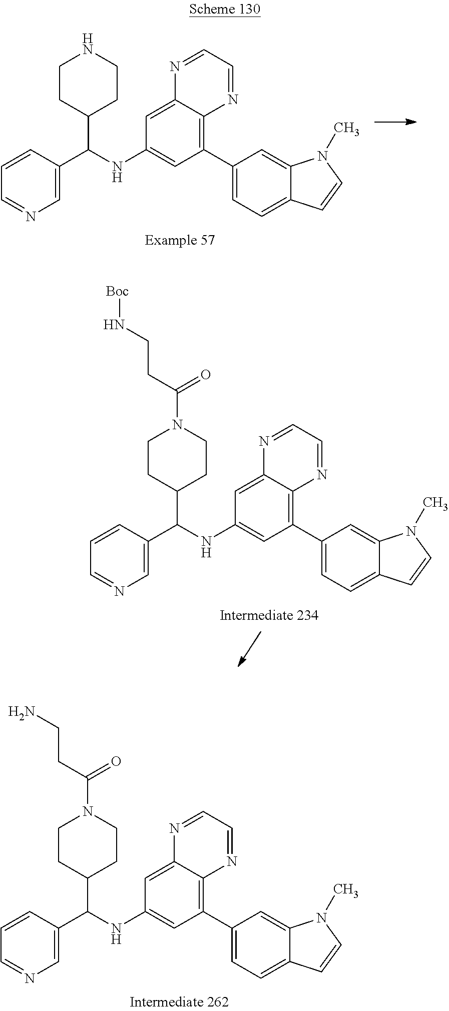 [SMILES: Cn1ccc2ccc(-c3cc(NC(c4cccnc4)C4CCNCC4)cc4nccnc34)cc21 ; Cn1ccc2ccc(-c3cc(NC(c4cccnc4)C4CCN(C(=O)CCNC(=O)OC(C)(C)C)CC4)cc4nccnc34)cc21 ; Cn1ccc2ccc(-c3cc(NC(c4cccnc4)C4CCN(C(=O)CCN)CC4)cc4nccnc34)cc21]

### Intermediate 234

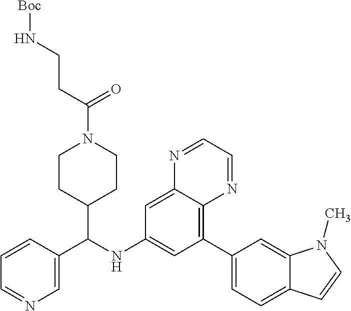 [SMILES: Cn1ccc2ccc(-c3cc(NC(c4cccnc4)C4CCN(C(=O)CCNC(=O)OC(C)(C)C)CC4)cc4nccnc34)cc21]

The product is prepared according to General Procedure 65, described for Intermediate 222 with 3-tert-Butoxycarbonylamino-propionic acid (27.84 mg; 0.15 mmol; 1.10 eq.), [8-(1-methyl-1H-indol-6-yl)-quinoxalin-6-yl]-(piperidin-4-yl-pyridin-3-yl-methyl)-amine (Example 57) (60.00 mg; 0.13 mmol; 1.00 eq.), DCC (30.36 mg; 0.15 mmol; 1.10 eq.) and DCM anhydrous (4 mL). Crude [3-(4-{[8-(1-methyl-1H-indol-6-yl)-quinoxalin-6-ylamino]-pyridin-3-yl-methyl}-piperidin-1-yl)-3-oxo-propyl]-carbamic acid tert-butyl ester (83.74 mg; yield 99.7%; yellow powder) is used in the next step.

### Example 262

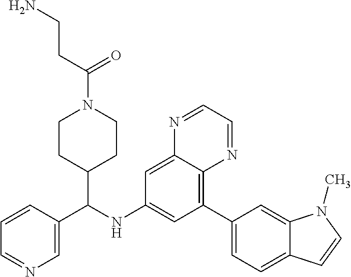 [SMILES: Cn1ccc2ccc(-c3cc(NC(c4cccnc4)C4CCN(C(=O)CCN)CC4)cc4nccnc34)cc21]

The product is prepared according to General Procedure 11, described in Example 46 with [3-(4-{[8-(1-methyl-1H-indol-6-yl)-quinoxalin-6-ylamino]-pyridin-3-yl-methyl}-piperidin-1-yl)-3-oxo-propyl]-carbamic acid tert-butyl ester (Intermediate 234) (88.00 mg; 0.14 mmol; 1.00 eq.), 2M HCl in Et₂O (2 mL) and DCM (3.00 mL). Purification by preparative HPLC (acidic conditions). 3-Amino-1-(4-{[8-(1-methyl-1H-indol-6-yl)-quinoxalin-6-ylamino]-pyridin-3-yl-methyl}-piperidin-1-yl)-propan-1-one formic acid (23.00 mg; yield 24.4%; 84% by HPLC) is obtained as a yellow powder.

### Example 263

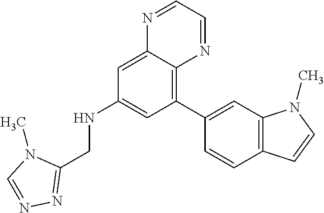 [SMILES: Cn1cnnc1CNc1cc(-c2ccc3ccn(C)c3c2)c2nccnc2c1]

The product is prepared according to General Procedure 6, described for Intermediate 6 with 7-chloro-5-(1-methyl-1H-indol-6-yl)-quinoxaline (Intermediate 4) (50.00 mg; 0.17 mmol; 1.00 eq.), C-(4-methyl-4H-[1,2,4]triazol-3-yl)-methylamine (76.35 mg; 0.68 mmol; 4.00 eq.), Cs₂CO₃ (443.67 mg; 1.36 mmol; 8.00 eq.), BINAP (31.80 mg; 0.05 mmol; 0.30 eq.), Pd(OAc)₂ (5.73 mg; 0.03 mmol; 0.15 eq.) and 1,4-dioxane (5 mL). Purification by FCC (NH₂ column; DCM/MeOH; gradient). [8-(1-Methyl-1H-indol-6-yl)-quinoxalin-6-yl]-(4-methyl-4H-[1,2,4]triazol-3-ylmethyl)-amine (33.00 mg; yield 49.4%; 94% by HPLC).

### Example 264

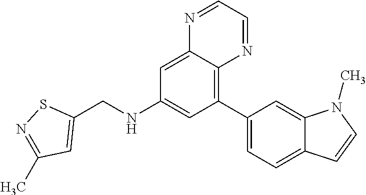 [SMILES: Cc1cc(CNc2cc(-c3ccc4ccn(C)c4c3)c3nccnc3c2)sn1]

The product is prepared according to General Procedure 23, described in Example 63 with 8-(1-methyl-1H-indol-6-yl)-quinoxalin-6-ylamine (Intermediate 22) (70.00 mg; 0.26 mmol; 1.00 eq.), 3-methyl-isothiazole-5-carbaldehyde (25.55 μL; 0.26 mmol; 1.00 eq.), Hantzsch ester (80.79 mg; 0.32 mmol; 1.25 eq.) and TMCS (6.48 μL; 0.05 mmol; 0.20 eq.) and DCM anhydrous (2 mL). Purification by FCC (DCM/MeOH; gradient) and repurification by preparative HPLC (acidic conditions). [8-(1-Methyl-1H-indol-6-yl)-quinoxalin-6-yl]-(3-methyl-isothiazol-5-ylmethyl)-amine (30.00 mg; yield 30.2%; 99% by HPLC) is obtained as a yellow powder.

### Example 265

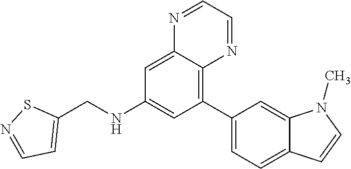 [SMILES: Cn1ccc2ccc(-c3cc(NCc4ccns4)cc4nccnc34)cc21]

The product is prepared according to General Procedure 23, described in Example 63 with 8-(1-methyl-1H-indol-6-yl)-quinoxalin-6-ylamine (Intermediate 22) (60.00 mg; 0.21 mmol; 1.00 eq.), isothiazole-5-carbaldehyde (24.25 mg; 0.21 mmol; 1.00 eq.), Hantzsch ester (67.87 mg; 0.27 mmol; 1.25 eq.), TMCS (5.44 μL; 0.04 mmol; 0.20 eq.) and DCM anhydrous (2.00 ml). Purification by FCC (hexane/EtOAc; gradient). Isothiazol-5-ylmethyl-[8-(1-methyl-1H-indol-6-yl)-quinoxalin-6-yl]-amine (14.30 mg; yield 15.8%; 87% by HPLC).

### Example 266

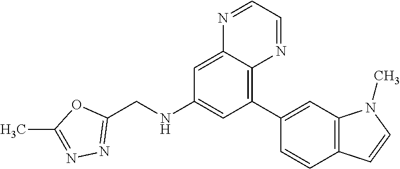 [SMILES: Cc1nnc(CNc2cc(-c3ccc4ccn(C)c4c3)c3nccnc3c2)o1]

The product is prepared according to General Procedure 6, described for Intermediate 6 with 7-chloro-5-(1-methyl-1H-indol-6-yl)-quinoxaline (Intermediate 4) (50.00 mg; 0.17 mmol; 1.00 eq.), C-(5-methyl-[1,3,4]oxadiazol-2-yl)-methylamine (28.88 mg; 0.26 mmol; 1.50 eq.), Cs₂CO₃ (168.06 mg; 0.51 mmol; 3.00 eq.), BINAP (10.81 mg; 0.02 mmol; 0.10 eq.), Pd(OAc)₂ (4.02 mg; 0.02 mmol; 0.10 eq.) and 1,4-dioxane (2 mL). Purification by FCC (DCM/MeOH; gradient) and repurification by preparative HPLC (basic conditions). [8-(1-Methyl-1H-indol-6-yl)-quinoxalin-6-yl]-(5-methyl-[1,3,4]oxadiazol-2-ylmethyl)-amine (13.00 mg; yield 15.9%; 97% by HPLC) is obtained as a yellow powder.

### Example 267

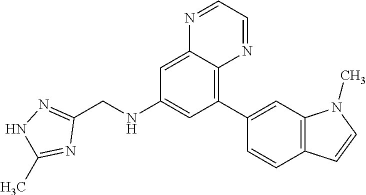 [SMILES: Cc1nc(CNc2cc(-c3ccc4ccn(C)c4c3)c3nccnc3c2)n[nH]1]

The product is prepared according to General Procedure 3, described in Example 18 with 7-chloro-5-(1-methyl-1H-indol-6-yl)-quinoxaline (Intermediate 4) (50.00 mg; 0.17 mmol; 1.00 eq.), C-(5-methyl-1H-[1,2,4]triazol-3-yl)-methylamine (20.53 mg; 0.18 mmol; 1.10 eq.), BrettPhos (4.47 mg; 0.01 mmol; 0.05 eq.), BrettPhos precatalyst (6.65 mg; 0.01 mmol; 0.05 eq.) and LiHMDS 1.0 M in THF (299.64 μL; 0.30 mmol; 1.80 eq.). Purification by FCC (hexane/EtOAc; gradient). [8-(1-Methyl-1H-indol-6-yl)-quinoxalin-6-yl]-(5-methyl-1H-[1,2,4]triazol-3-ylmethyl)-amine (15.00 mg; yield 23.8%; 97% by HPLC) is obtained as a yellow powder.

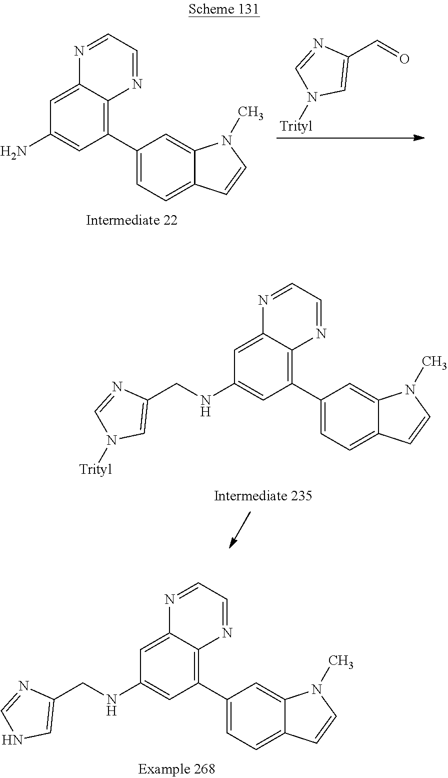

### Intermediate 235

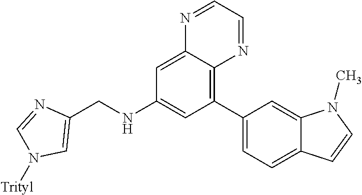

The product is prepared according to General Procedure 23, described in Example 63 with 8-(1-methyl-1H-indol-6-yl)-quinoxalin-6-ylamine (Intermediate 22) (60.00 mg; 0.21 mmol; 1.00 eq.), 3-trityl-3H-imidazole-4-carbaldehyde (72.54 mg; 0.21 mmol; 1.00 eq.), Hantzsch ester (67.87 mg; 0.27 mmol; 1.25 eq.) and TMCS (5.44 μL; 0.04 mmol; 0.20 eq.) and DCM anhydrous (2 mL). Purification by FCC (hexane/EtOAc; gradient). [8-(1-Methyl-1H-indol-6-yl)-quinoxalin-6-yl]-(3-trityl-3H-imidazol-4-ylmethyl)-amine (66.00 mg; yield 51.1%; 100% by UPLC) is obtained as a yellow powder.

### Example 268

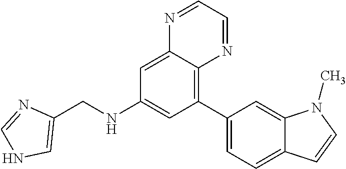 [SMILES: Cn1ccc2ccc(-c3cc(NCc4c[nH]cn4)cc4nccnc34)cc21]

The product is prepared according to General Procedure 10, described in Example 44 with [8-(1-methyl-1H-indol-6-yl)-quinoxalin-6-yl]-(3-trityl-3H-imidazol-4-ylmethyl)-amine (Intermediate 235) (66.00 mg; 0.11 mmol; 1.00 eq.), TFA (2.00 mL; 20.00 mmol; 182.61 eq.) and DCM (3 mL). Purification by FCC (NH₂ column; EtOAc/MeOH; gradient). (3H-Imidazol-4-ylmethyl)-[8-(1-methyl-1H-indol-6-yl)-quinoxalin-6-yl]-amine (4.80 mg; yield 10.7%) is obtained as a yellow powder.

### Example 269

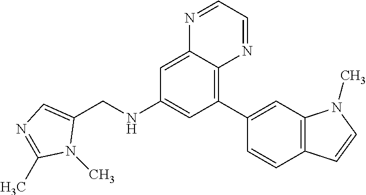 [SMILES: Cc1ncc(CNc2cc(-c3ccc4ccn(C)c4c3)c3nccnc3c2)n1C]

The product is prepared according to General Procedure 23, described in Example 63 with 8-(1-methyl-1H-indol-6-yl)-quinoxalin-6-ylamine (Intermediate 22) (70.00 mg; 0.25 mmol; 1.00 eq.), 2,3-dimethyl-3H-imidazole-4-carbaldehyde (31.04 mg; 0.25 mmol; 1.00 eq.), Hantzsch ester (79.18 mg; 0.31 mmol; 1.25 eq.), TMCS (6.35 μL; 0.05 mmol; 0.20 eq.) and DCM anhydrous (2 mL). Purification by FCC (DCM/MeOH; gradient) and repurification by preparative HPLC (acidic conditions). (2,3-Dimethyl-3H-imidazol-4-ylmethyl)-[8-(1-methyl-1H-indol-6-yl)-quinoxalin-6-yl]-amine formic acid (65.00 mg; yield 60.5%; 99% by HPLC) is obtained as a yellow powder.

### Example 270

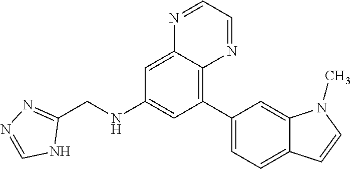 [SMILES: Cn1ccc2ccc(-c3cc(NCc4nnc[nH]4)cc4nccnc34)cc21]

The product is prepared according to General Procedure 23, described in Example 63 with 7-chloro-5-(1-methyl-1H-indol-6-yl)-quinoxaline (75.00 mg; 0.25 mmol; 1.00 eq.), BrettPhos (9.40 mg; 0.02 mmol; 0.07 eq.), BrettPhos precatalyst (13.99 mg; 0.02 mmol; 0.07 eq.), C-(1H-[1,2,4]triazol-3-yl)-methylamine (0.03 mL; 0.30 mmol; 1.20 eq.) and LiHMDS 1M THF solution (600.51 μL; 0.60 mmol; 2.40 eq.). Purification by FCC (column deactivated with 1% Et₃N/DCM and DCM; DCM/MeOH; gradient) and repurification by preparative HPLC (acidic conditions). [8-(1-Methyl-1H-indol-6-yl)-quinoxalin-6-yl]-(1H-[1,2,4]triazol-3-ylmethyl)-amine formic acid (10.50 mg; yield 10.2%; 98% by HPLC) is obtained as a yellow powder.

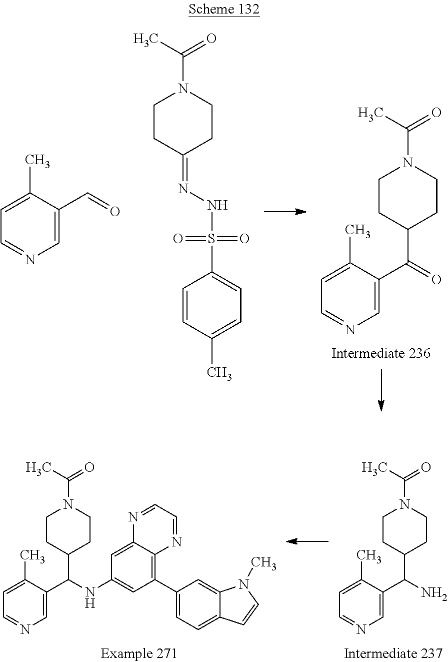

### Intermediate 236

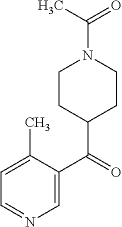 [SMILES: CC(=O)N1CCC(C(=O)c2cnccc2C)CC1]

The product is prepared according to General Procedure 15, described for Intermediate 13 with N-[(1-acetyl-4-piperidylidene)amino]-4-methyl-benzenesulfonamide (450.00 mg; 1.41 mmol; 1.00 eq.), 4-methyl-pyridine-3-carbaldehyde (290.84 mg; 2.40 mmol; 1.70 eq.), Cs₂CO₃ (2760.97 mg; 8.47 mmol; 6.00 eq.). Purification by FCC (DCM/MeOH; gradient). 1-[4-(4-Methyl-pyridine-3-carbonyl)-piperidin-1-yl]-ethanone (85.90 mg; yield 19.2%; 77% by UPLC) is obtained as a yellow oil.

### Intermediate 237

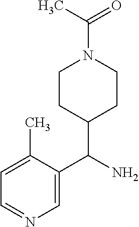 [SMILES: CC(=O)N1CCC(C(N)c2cnccc2C)CC1]

The product is prepared according to General Procedure 14, described for Intermediate 12 with 1-[4-(4-methyl-pyridine-3-carbonyl)-piperidin-1-yl]-ethanone (Intermediate 236) (85.90 mg; 0.27 mmol; 1.00 eq.), TTIP (0.16 mL; 0.54 mmol; 2.00 eq.), NaBH₄ (41.01 mg; 1.08 mmol; 4.00 eq.) and 7M NH₃ in MeOH (4 mL). Crude 1-{4-[amino-(4-methyl-pyridin-3-yl)-methyl]-piperidin-1-yl}-ethanone (80.00 mg; yield 72.8%; white-yellow solid) is used in the next step.

### Example 271

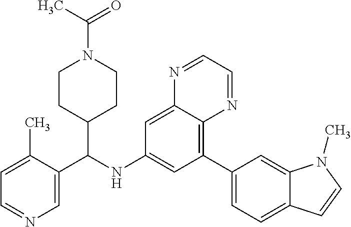 [SMILES: CC(=O)N1CCC(C(Nc2cc(-c3ccc4ccn(C)c4c3)c3nccnc3c2)c2cnccc2C)CC1]

The product is prepared according to General Procedure 12, described in Example 1 with 7-chloro-5-(1-methyl-1H-indol-6-yl)-quinoxaline (Intermediate 4) (50.00 mg; 0.17 mmol; 1.00 eq.), 1-{4-[amino-(4-methyl-pyridin-3-yl)-methyl]-piperidin-1-yl}-ethanone (Intermediate 237) (101.45 mg; 0.25 mmol; 1.50 eq.), NaOtBu (56.11 mg; 0.58 mmol; 3.50 eq.), Pd₂(dba)₃ (30.55 mg; 0.03 mmol; 0.20 eq.), BINAP (41.55 mg; 0.07 mmol; 0.40 eq.) and toluene (3 mL). Purification by FCC (NH₂ column; hexane/EtOAc; gradient then EtOAc/MeOH; gradient). 1-{4-[[8-(1-Methyl-1H-indol-6-yl)-quinoxalin-6-ylamino]-(4-methyl-pyridin-3-yl)-methyl]-piperidin-1-yl}-ethanone (7.50 mg; yield 8.5%; 95% by HPLC) is obtained as a yellow powder.

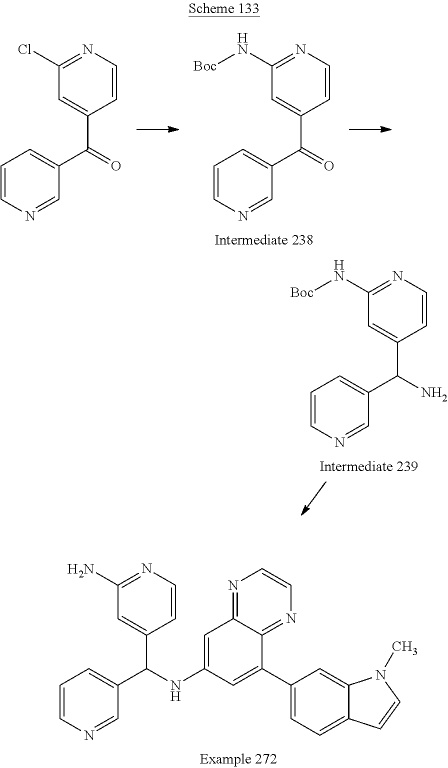 [SMILES: O=C(c1cccnc1)c1ccnc(Cl)c1 ; CC(C)(C)OC(=O)Nc1cc(C(=O)c2cccnc2)ccn1 ; CC(C)(C)OC(=O)Nc1cc(C(N)c2cccnc2)ccn1 ; Cn1ccc2ccc(-c3cc(NC(c4cccnc4)c4ccnc(N)c4)cc4nccnc34)cc21]

### Intermediate 238

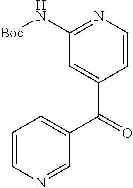 [SMILES: CC(C)(C)OC(=O)Nc1cc(C(=O)c2cccnc2)ccn1]

The product is prepared according to General Procedure 33, described in Example 78 with (2-chloro-pyridin-4-yl)-pyridin-3-yl-methanone (200.00 mg; 0.91 mmol; 1.00 eq.), Cs₂CO₃ (596.09 mg; 1.83 mmol; 2.00 eq.), carbamic acid tert-butyl ester (160.74 mg; 1.37 mmol; 1.50 eq.), Pd₂(dba)₃ (17.63 mg; 0.02 mmol; 0.02 eq.), XantPhoS (31.76 mg; 0.05 mmol; 0.06 eq.) and 1,4-dioxane (1 mL) was added. Purification by FCC (DCM/MeOH; gradient). [4-(Pyridine-3-carbonyl)-pyridin-2-yl]-carbamic acid tert-butyl ester (165.00 mg; yield 60.3%) is obtained as a light yellow oil.

### Intermediate 239

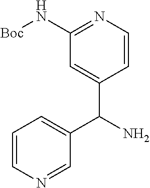 [SMILES: CC(C)(C)OC(=O)Nc1cc(C(N)c2cccnc2)ccn1]

The product is prepared according to General Procedure 14, described for Intermediate 12 with [4-(pyridine-3-carbonyl)-pyridin-2-yl]-carbamic acid tert-butyl ester (Intermediate 238) (165.00 mg; 0.55 mmol; 1.00 eq.), TTIP (0.33 ml; 1.10 mmol; 2.00 eq.), NaBH₄ (83.42 mg; 2.20 mmol; 4.00 eq.) and 7M NH₃ in MeOH (50 mL). Crude product, [4-(amino-pyridin-3-yl-methyl)-pyridin-2-yl]-carbamic acid tert-butyl ester (120.00 mg; yield 64.8%; yellow foam) is used directly in the next step, without further purification.

### Example 272

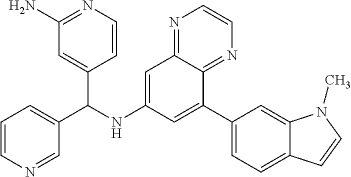 [SMILES: Cn1ccc2ccc(-c3cc(NC(c4cccnc4)c4ccnc(N)c4)cc4nccnc34)cc21]

The product is prepared according to General Procedure 2, described in Example 1 with 7-chloro-5-(1-methyl-1H-indol-6-yl)-quinoxaline (Intermediate 12) (90.00 mg; 0.31 mmol; 1.00 eq.), [4-(amino-pyridin-3-yl-methyl)-pyridin-2-yl]-carbamic acid tert-butyl ester (Intermediate 239) (119.63 mg; 0.40 mmol; 1.30 eq.), NaOtBu (76.47 mg; 0.80 mmol; 2.60 eq.), Pd₂(dba)₃ (28.06 mg; 0.03 mmol; 0.10 eq.), BINAP (38.16 mg; 0.06 mmol; 0.20 eq.) and toluene (5 mL). Purification by FCC (NH₂ column; DCM/MeOH; gradient). [(2-Amino-pyridin-4-yl)-pyridin-3-yl-methyl]-[8-(1-methyl-1H-indol-6-yl)-quinoxalin-6-yl]-amine (12.00 mg; yield 7.3%; 85% by HPLC) is obtained as a dark yellow powder.

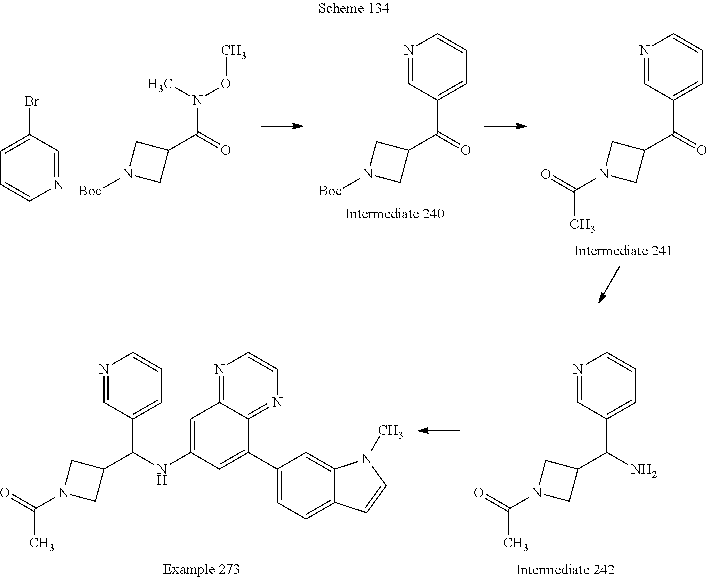

### Intermediate 240

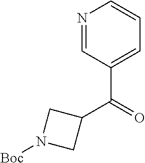 [SMILES: CC(C)(C)OC(=O)N1CC(C(=O)c2cccnc2)C1]

The product is prepared according to General Procedure 60, described for Intermediate 198 with 3-promo-pyridine (0.79 mL; 8.19 mmol; 4.00 eq.), Isopropylmagnesium chloride 2M in THF (4.09 mL; 8.19 mmol; 4.00 eq.), 3-(methoxy-methyl-carbamoyl)-azetidine-1-carboxylic acid tert-butyl ester (500.00 mg; 2.05 mmol; 1.00 eq.) and anhydrous THF (8 mL). Purification by FCC (DCM/MeOH; gradient). 3-(Pyridine-3-carbonyl)-azetidine-1-carboxylic acid tert-butyl ester (0.47 g; yield 85.8%) is obtained as a yellow oil.

### Intermediate 241

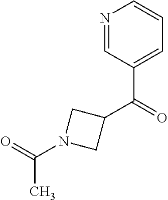

The product is prepared according to General Procedure 10, described in Example 44 with 3-(pyridine-3-carbonyl)-azetidine-1-carboxylic acid tert-butyl ester (Intermediate 240) (480.00 mg; 1.78 mmol; 1.00 eq.), TFA (0.89 mL; 8.88 mmol; 5.00 eq.) and DCM anhydrous (14 mL). Then according to General Procedure 35, described in Example 82 with DIPEA (1.55 mL; 8.88 mmol; 5.00 eq.), acetyl chloride (257.57 μL; 3.55 mmol; 2.00 eq.) and DCM anhydrous (14 mL). Purification by FCC (DCM/MeOH; gradient). 1-[3-(Pyridine-3-carbonyl)-azetidin-1-yl]-ethanone (0.66 g; yield 155.0%) is obtained as a dark oil.

### Intermediate 242

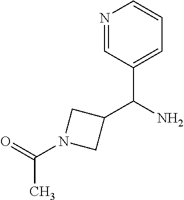

The product is prepared according to General Procedure 14, described for Intermediate 12 with 1-[3-(pyridine-3-carbonyl)-azetidin-1-yl]-ethanone (Intermediate 241) (0.66 g; 2.71 mmol; 1.00 eq.), TTIP (1.61 ml; 5.43 mmol; 2.00 eq.), NaBH₄ (410.81 mg; 10.86 mmol; 4.00 eq.) and 7M NH₃ in MeOH (14.20 mL; 99.38 mmol; 36.61 eq.). Crude 1-[3-(amino-pyridin-3-yl-methyl)-azetidin-1-yl]-ethanone (0.54 g; yield 89.8%; yellow oil) is used in the next step.

### Example 273

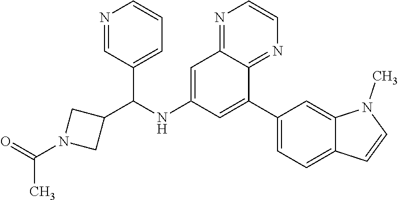 [SMILES: CC(=O)N1CC(C(Nc2cc(-c3ccc4ccn(C)c4c3)c3nccnc3c2)c2cccnc2)C1]

The product is prepared according to General Procedure 2, described in Example 1 with 1-[3-(amino-pyridin-3-yl-methyl)-azetidin-1-yl]-ethanone (Intermediate 242) (0.24 g; 1.08 mmol; 5.27 eq.), 7-chloro-5-(1-methyl-1H-indol-6-yl)-quinoxaline (Intermediate 4) (60.00 mg; 0.20 mmol; 1.00 eq.), NaOtBu (48.00 mg; 0.50 mmol; 2.45 eq.), BINAP (14.80 mg; 0.02 mmol; 0.12 eq.), Pd₂(dba)₃ (11.00 mg; 0.01 mmol; 0.06 eq.) and 1,4-dioxane (1.50 mL). Purification by FCC (DCM/MeOH; gradient). 1-(3-{[8-(1-Methyl-1H-indol-6-yl)-quinoxalin-6-ylamino]-pyridin-3-yl-methyl}-azetidin-1-yl)-ethanone (14.60 mg; yield 15.5%; 99% by HPLC) is obtained as a yellow solid.

### Example 274

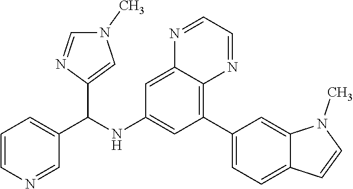 [SMILES: Cn1cnc(C(Nc2cc(-c3ccc4ccn(C)c4c3)c3nccnc3c2)c2cccnc2)c1]

The product is prepared according to General Procedure 2, described in Example 1 with 7-chloro-5-(1-methyl-1H-indol-6-yl)-quinoxaline (Intermediate 4) (50.00 mg; 0.17 mmol; 1.00 eq.), C-(1-methyl-1H-imidazol-4-yl)-C-pyridin-3-yl-methylamine hydrochloride (58.44 mg; 0.22 mmol; 1.30 eq.), NaOtBu (47.99 mg; 0.50 mmol; 3.00 eq.), BINAP (15.55 mg; 0.02 mmol; 0.15 eq.), Pd₂(dba)₃ (7.62 mg; 0.01 mmol; 0.05 eq.), toluene (3 mL) and 1,4-dioxane (0.50 mL). Purification by FCC (hexane/EtOAc; gradient and then EtOAc/MeOH; gradient). [(1-Methyl-1H-imidazol-4-yl)-pyridin-3-yl-methyl]-[8-(1-methyl-1H-indol-6-yl)-quinoxalin-6-yl]-amine (3.00 mg; yield 3.5%; 87% by HPLC) is obtained as a yellow solid.

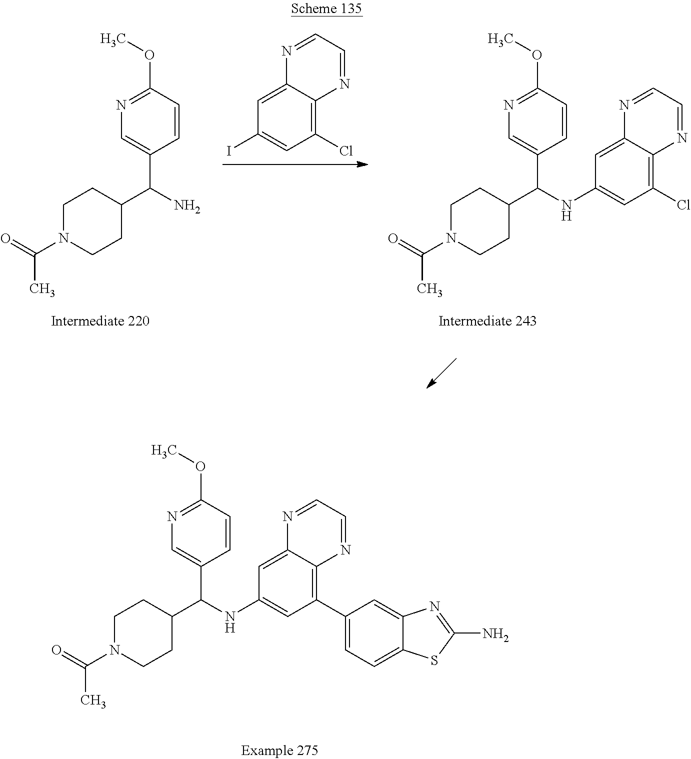 [SMILES: COc1ccc(C(N)C2CCN(C(C)=O)CC2)cn1 ; Clc1cc(I)cc2nccnc12 ; COc1ccc(C(Nc2cc(Cl)c3nccnc3c2)C2CCN(C(C)=O)CC2)cn1 ; COc1ccc(C(Nc2cc(-c3ccc4sc(N)nc4c3)c3nccnc3c2)C2CCN(C(C)=O)CC2)cn1]

### Intermediate 243

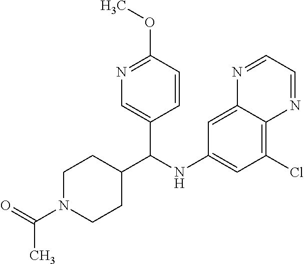 [SMILES: COc1ccc(C(Nc2cc(Cl)c3nccnc3c2)C2CCN(C(C)=O)CC2)cn1]

The product is prepared according to General Procedure 2, described in Example 1 with 7-chloro-5-(1-methyl-1H-indol-6-yl)-quinoxaline (Intermediate 4) (100.00 mg; 0.34 mmol; 1.00 eq.), 1-{4-[amino-(6-methoxy-pyridin-3-yl)-methyl]-piperidin-1-yl}-ethanone (Intermediate 220) (123.62 mg; 0.36 mmol; 1.05 eq.), BINAP (42.87 mg; 0.07 mmol; 0.20 eq.), Pd₂(dba)₃ (31.52 mg; 0.03 mmol; 0.10 eq.), NaOtBu (82.62 mg; 0.86 mmol; 2.50 eq.) and toluene (5 mL). Purification by FCC (hexane/EtOAc; gradient). 1-{4-[(8-Chloro-quinoxalin-6-ylamino)-(6-methoxy-pyridin-3-yl)-methyl]-piperidin-1-yl}-ethanone (66.00 mg; yield 40.0%; 88% by UPLC) is obtained as a yellow powder.

### Example 275

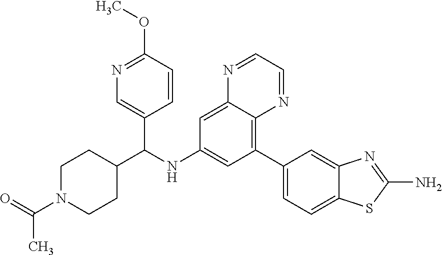 [SMILES: COc1ccc(C(Nc2cc(-c3ccc4sc(N)nc4c3)c3nccnc3c2)C2CCN(C(C)=O)CC2)cn1]

The product is prepared according to General Procedure 28, described in Example 71 with 1-{4-[(8-chloro-quinoxalin-6-ylamino)-(6-methoxy-pyridin-3-yl)-methyl]-piperidin-1-yl}-ethanone (Intermediate 243) (40.00 mg; 0.08 mmol; 1.00 eq.), 5-(4,4,5,5-tetramethyl-[1,3,2]dioxaborolan-2-yl)-benzothiazol-2-ylamine (35.76 mg; 0.09 mmol; 1.10 eq.), Na₂CO₃ (44.30 mg; 0.42 mmol; 5.00 eq.), Pd(PPh₃)₄ (5.08 mg; 0.00 mmol; 0.05 eq.), toluene (2 mL), EtOH (1 mL) and water (1 mL). Purification by FCC (EtOAc/MeOH; gradient). 1-{4-[[8-(2-Amino-benzothiazol-5-yl)-quinoxalin-6-ylamino]-(6-methoxy-pyridin-3-yl)-methyl]-piperidin-1-yl}-ethanone (6.00 mg; yield 11.7%; by HPLC) is obtained as a yellow powder.

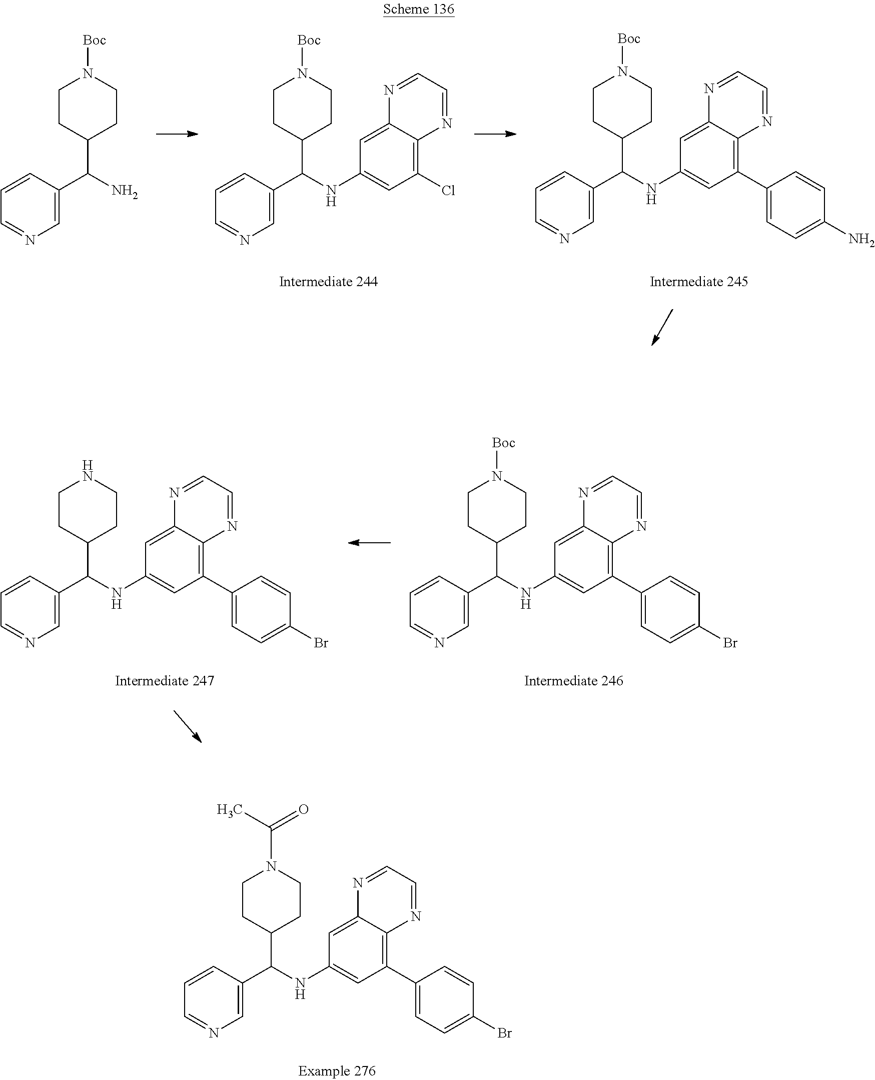 [SMILES: CC(C)(C)OC(=O)N1CCC(C(N)c2cccnc2)CC1 ; CC(C)(C)OC(=O)N1CCC(C(Nc2cc(Cl)c3nccnc3c2)c2cccnc2)CC1 ; CC(C)(C)OC(=O)N1CCC(C(Nc2cc(-c3ccc(N)cc3)c3nccnc3c2)c2cccnc2)CC1 ; Brc1ccc(-c2cc(NC(c3cccnc3)C3CCNCC3)cc3nccnc23)cc1 ; CC(C)(C)OC(=O)N1CCC(C(Nc2cc(-c3ccc(Br)cc3)c3nccnc3c2)c2cccnc2)CC1 ; CC(=O)N1CCC(C(Nc2cc(-c3ccc(Br)cc3)c3nccnc3c2)c2cccnc2)CC1]

### Intermediate 244

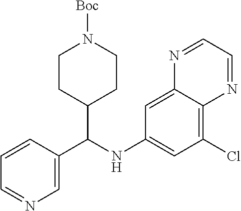 [SMILES: CC(C)(C)OC(=O)N1CCC(C(Nc2cc(Cl)c3nccnc3c2)c2cccnc2)CC1]

The product is prepared according to General Procedure 2, described in Example 1 with 5-chloro-7-iodo-quinoxaline (195.00 mg; 0.66 mmol; 1.00 eq.), 4-(amino-pyridin-3-yl-methyl)-piperidine-1-carboxylic acid tert-butyl ester (Intermediate 14) (228.72 mg; 0.70 mmol; 1.05 eq.), BINAP (82.76 mg; 0.13 mmol; 0.20 eq.), Pd₂(dba)₃ (60.86 mg; 0.07 mmol; 0.10 eq.), NaOtBu (159.50 mg; 1.66 mmol; 2.50 eq.) and toluene (5 mL). Purification by FCC (EtOAc/MeOH; gradient). 4-[(8-Chloro-quinoxalin-6-ylamino)-pyridin-3-yl-methyl]-piperidine-1-carboxylic acid tert-butyl ester (134.00 mg; yield 43.9%; 98.8% by UPLC) is obtained as a yellow powder.

### Intermediate 245

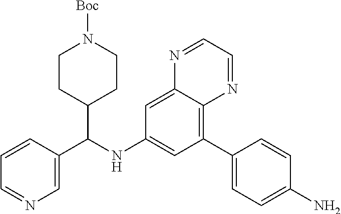 [SMILES: CC(C)(C)OC(=O)N1CCC(C(Nc2cc(-c3ccc(N)cc3)c3nccnc3c2)c2cccnc2)CC1]

The product is prepared according to General Procedure 28, described in Example 71 with 4-[(8-chloro-quinoxalin-6-ylamino)-pyridin-3-yl-methyl]-piperidine-1-carboxylic acid tert-butyl ester (Intermediate 244) (60.00 mg; 0.13 mmol; 1.00 eq.), Na₂CO₃ (70.04 mg; 0.66 mmol; 5.00 eq.), 4-(4,4,5,5-Tetramethyl-[1,3,2]dioxaborolan-2-yl)-phenylamine (43.43 mg; 0.20 mmol; 1.50 eq.), Pd(PPh₃)₄ (7.64 mg; 0.01 mmol; 0.05 eq.), toluene (1 mL), EtOH (0.5 mL) and water (0.5 mL). Purification by FCC (DCM/MeOH; gradient). 4-{[8-(4-Amino-phenyl)-quinoxalin-6-ylamino]-pyridin-3-yl-methyl}-piperidine-1-carboxylic acid tert-butyl ester (130.00 mg; yield 156.6%) is obtained as a yellow powder.

### Intermediate 246

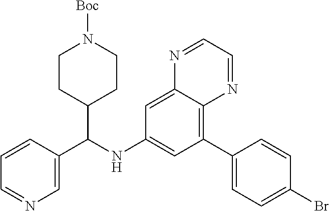 [SMILES: CC(C)(C)OC(=O)N1CCC(C(Nc2cc(-c3ccc(Br)cc3)c3nccnc3c2)c2cccnc2)CC1]

In round bottom flask anhydrous Copper(II) bromide (68.24 mg; 0.31 mmol; 1.20 eq.), tert-butyl nitrite (45.42 μL; 0.38 mmol; 1.50 eq.), anhydrous ACN (5.00 ml) (degassed) are placed. RM is cooled to 0° C. and 4-{[8-(4-amino-phenyl)-quinoxalin-6-ylamino]-pyridin-3-yl-methyl}-piperidine-1-carboxylic acid tert-butyl ester (Intermediate 245) (130.00 mg; 0.25 mmol; 1.00 eq.) in 1,4-dioxane (5 mL) is slowly added. The reaction is stirred at rt for 2 h. The reaction mixture is extracted with DCM. The organic phase is washed with brine, dried (sodium sulfate) and evaporated. Purification by FCC (NH₂ column; DCM/MeOH; gradient). 4-{[8-(4-Bromo-phenyl)-quinoxalin-6-ylamino]-pyridin-3-yl-methyl}-piperidine-1-carboxylic acid tert-butyl ester (60.00 mg; yield 7.8%) is obtained as a yellow powder.

### Intermediate 247

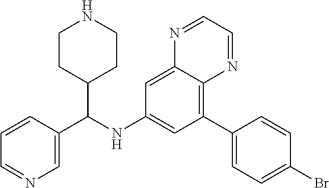 [SMILES: Brc1ccc(-c2cc(NC(c3cccnc3)C3CCNCC3)cc3nccnc23)cc1]

The product is prepared according to General Procedure 10, described in Example 44 with 4-{[8-(4-Bromo-phenyl)-quinoxalin-6-ylamino]-pyridin-3-yl-methyl}-piperidine-1-carboxylic acid tert-butyl ester (Intermediate 246) (60.00 mg; 0.10 mmol; 1.00 eq.), TFA (1 mL) and DCM (3 mL). Crude [8-(4-Bromo-phenyl)-quinoxalin-6-yl]-(piperidin-4-yl-pyridin-3-yl-methyl)-amine (30.00 mg; yield 16.3%; yellow) is used in to the next step without further purification.

### Example 276

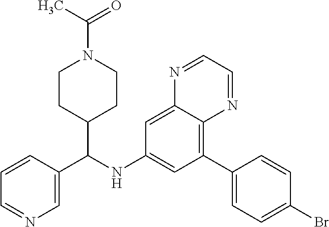 [SMILES: CC(=O)N1CCC(C(Nc2cc(-c3ccc(Br)cc3)c3nccnc3c2)c2cccnc2)CC1]

The product is prepared according to General Procedure 16, described for Intermediate 17 with [8-(4-bromo-phenyl)-quinoxalin-6-yl]-(piperidin-4-yl-pyridin-3-yl-methyl)-amine (Intermediate 247) (30.00 mg; 0.06 mmol; 1.00 eq.), TEA (20.51 μL; 0.16 mmol; 2.50 eq.), acetic anhydride (6.58 μL; 0.07 mmol; 1.10 eq.) and DCM anhydrous (10 mL). Purification by preparative HPLC (acidic conditions). 1-(4-{[8-(4-Bromo-phenyl)-quinoxalin-6-ylamino]-pyridin-3-yl-methyl}-piperidin-1-yl)-ethanone (3.30 mg; yield 10.1%; 93% by HPLC) is obtained as a yellow powder.

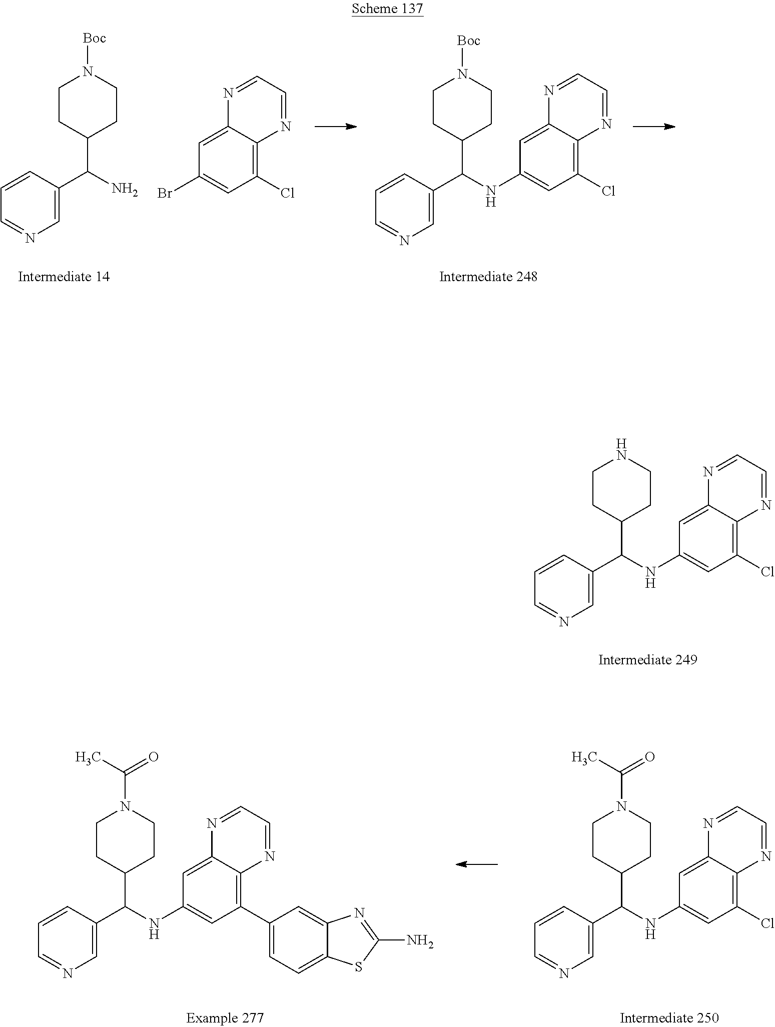 [SMILES: CC(C)(C)OC(=O)N1CCC(C(N)c2cccnc2)CC1 ; Clc1cc(Br)cc2nccnc12 ; CC(C)(C)OC(=O)N1CCC(C(Nc2cc(Cl)c3nccnc3c2)c2cccnc2)CC1 ; Clc1cc(NC(c2cccnc2)C2CCNCC2)cc2nccnc12 ; CC(=O)N1CCC(C(Nc2cc(-c3ccc4sc(N)nc4c3)c3nccnc3c2)c2cccnc2)CC1 ; CC(=O)N1CCC(C(Nc2cc(Cl)c3nccnc3c2)c2cccnc2)CC1]

### Intermediate 248

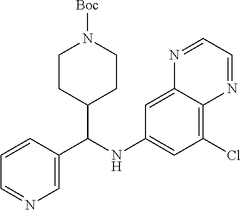 [SMILES: CC(C)(C)OC(=O)N1CCC(C(Nc2cc(Cl)c3nccnc3c2)c2cccnc2)CC1]

The product is prepared according to General Procedure 2, described in Example 1 with 4-(amino-pyridin-3-yl-methyl)-piperidine-1-carboxylic acid tert-butyl ester (Intermediate 14) (161.56 mg; 0.55 mmol; 1.50 eq.), 7-bromo-5-chloro-quinoxaline (Intermediate 3) (90.00 mg; 0.37 mmol; 1.00 eq.), NaOtBu (88.81 mg; 0.92 mmol; 2.50 eq.), BINAP (46.03 mg; 0.07 mmol; 0.20 eq.), Pd₂(dba)₃ (33.85 mg; 0.04 mmol; 0.10 eq.) and toluene (3 mL). Purification by FCC (DCM/MeOH; gradient). 4-[(8-Chloro-quinoxalin-6-ylamino)-pyridin-3-yl-methyl]-piperidine-1-carboxylic acid tert-butyl ester (90.00 mg; yield 53.6%) is obtained as a yellow powder.

### Intermediate 249

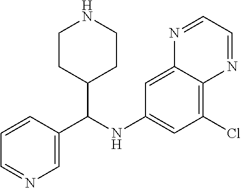 [SMILES: Clc1cc(NC(c2cccnc2)C2CCNCC2)cc2nccnc12]

The product is prepared according to General Procedure 11, described in Example 46 with 4-[(8-chloro-quinoxalin-6-ylamino)-pyridin-3-yl-methyl]-piperidine-1-carboxylic acid tert-butyl ester (Intermediate 248) (75.00 mg; 0.16 mmol; 1.00 eq.), 2M HCl in Et₂O (2 mL) and DCM anhydrous (2 mL). Crude (8-chloro-quinoxalin-6-yl)-(piperidin-4-yl-pyridin-3-yl-methyl)-amine (57.00 mg; yield 98.7%; yellow powder, 100% by UPLC) is used in the next step.

### Intermediate 250

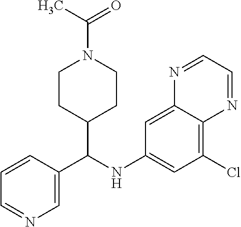 [SMILES: CC(=O)N1CCC(C(Nc2cc(Cl)c3nccnc3c2)c2cccnc2)CC1]

The product is prepared according to General Procedure 65, described for Intermediate 222 with (8-chloro-quinoxalin-6-yl)-(piperidin-4-yl-pyridin-3-yl-methyl)-amine (Intermediate 249) (57.90 mg; 0.16 mmol; 1.00 eq.), DCC (36.77 mg; 0.18 mmol; 1.10 eq.), CH₃COOH (10.20 μL; 0.18 mmol; 1.10 eq.) and DCM anhydrous (3 mL). Crude 1-{4-[(8-chloro-quinoxalin-6-ylamino)-pyridin-3-yl-methyl]-piperidin-1-yl}-ethanone (117.00 mg; yield 180.1%; yellow powder; 98% by UPLC) is used in the next step.

### Example 277

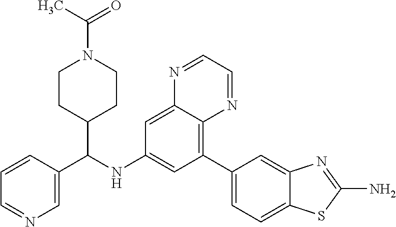 [SMILES: CC(=O)N1CCC(C(Nc2cc(-c3ccc4sc(N)nc4c3)c3nccnc3c2)c2cccnc2)CC1]

The product is prepared according to General Procedure 28, described in Example 71 with 1-{4-[(8-chloro-quinoxalin-6-ylamino)-pyridin-3-yl-methyl]-piperidin-1-yl}-ethanone (Intermediate 250) (40.00 mg; 0.10 mmol; 1.00 eq.), 5-(4,4,5,5-tetramethyl-[1,3,2]dioxaborolan-2-yl)-benzothiazol-2-ylamine (74.43 mg; 0.11 mmol; 1.10 eq.), Na₂CO₃ (51.94 mg; 0.49 mmol; 5.00 eq.), Pd(PPh₃)₄ (11.92 mg; 0.01 mmol; 0.10 eq.), toluene (2 mL), EtOH (1 mL) and water (1 mL). Purification by FCC (DCM/MeOH; gradient). 1-(4-{[8-(2-Amino-benzothiazol-5-yl)-quinoxalin-6-ylamino]-pyridin-3-yl-methyl}-piperidin-1-yl)-ethanone (6.00 mg; yield 11.8%; 91% by HPLC) is obtained as a yellow powder.

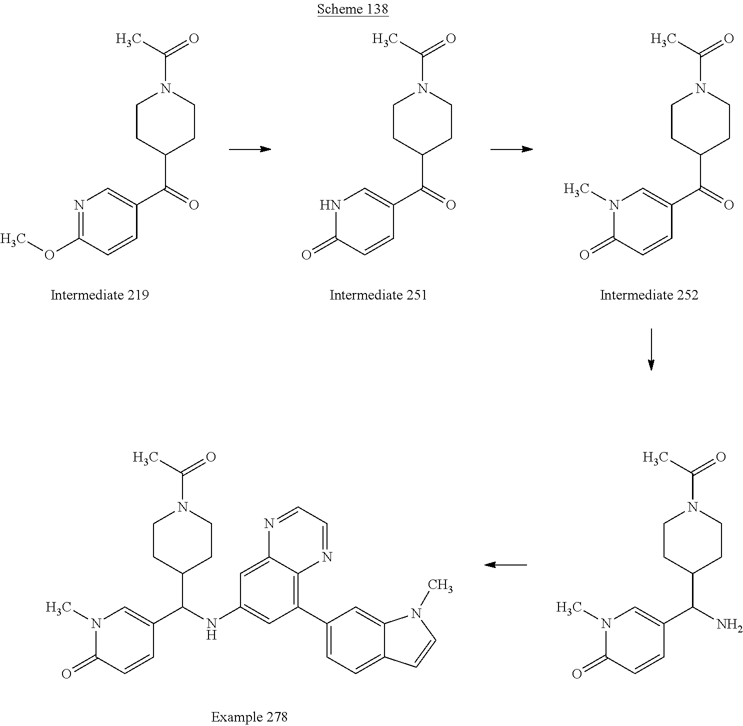 [SMILES: COc1ccc(C(=O)C2CCN(C(C)=O)CC2)cn1 ; CC(=O)N1CCC(C(=O)c2ccc(=O)[nH]c2)CC1 ; CC(=O)N1CCC(C(=O)c2ccc(=O)n(C)c2)CC1 ; CC(=O)N1CCC(C(Nc2cc(-c3ccc4ccn(C)c4c3)c3nccnc3c2)c2ccc(=O)n(C)c2)CC1 ; CC(=O)N1CCC(C(N)c2ccc(=O)n(C)c2)CC1]

### Intermediate 251

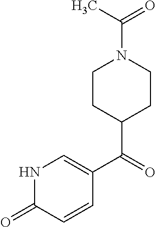 [SMILES: CC(=O)N1CCC(C(=O)c2ccc(=O)[nH]c2)CC1]

The suspension of 1-[4-(6-methoxy-pyridine-3-carbonyl)-piperidin-1-yl]-ethanone (Intermediate 219) (500.00 mg; 1.62 mmol; 1.00 eq.) and methylsulfanylsodium (1.14 g; 16.20 mmol; 10.00 eq.) in DMF (8 mL) is stirred for 48 h at 60° C. TFA (0.5 mL) is added, and the mixture is evaporated to dryness. The yellow oily residue is purified by FCC (EtOAc/MeOH; gradient). 1-[4-(6-Hydroxy-pyridine-3-carbonyl)-piperidin-1-yl]-ethanone (305.00 mg; yield 75.8%; 100% by UPLC) is obtained as a yellow powder.

### Intermediate 252

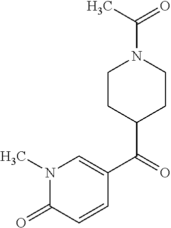 [SMILES: CC(=O)N1CCC(C(=O)c2ccc(=O)n(C)c2)CC1]

The product is prepared according to General Procedure 40 described for Intermediate 38 with 1-[4-(6-hydroxy-pyridine-3-carbonyl)-piperidin-1-yl]-ethanone (Intermediate 251) (150.00 mg; 0.60 mmol; 1.00 eq.), K₂CO₃ (166.99 mg; 1.21 mmol; 2.00 eq.), CH₃I (0.05 mL; 0.66 mmol; 1.10 eq.) and DMA (2 mL). Crude 5-(1-acetyl-piperidine-4-carbonyl)-1-methyl-1H-pyridin-2-one (171.00 mg; yield 95.7%; yellow oil; 88% by UPLC) is used in the next step.

### Intermediate 253

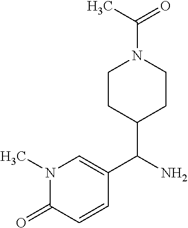 [SMILES: CC(=O)N1CCC(C(N)c2ccc(=O)n(C)c2)CC1]

The product is prepared according to General Procedure 14 described for Intermediate 12 with 5-(1-acetyl-piperidine-4-carbonyl)-1-methyl-1H-pyridin-2-one (140.00 mg; 0.53 mmol; 1.00 eq.), TTIP (0.32 mL; 1.07 mmol; 2.00 eq.), NaBH₄ (80.77 mg; 2.13 mmol; 4.00 eq.) and 7M NH₃ in MeOH (4 mL). Crude 5-[(1-acetyl-piperidin-4-yl)-amino-methyl]-1-methyl-1H-pyridin-2-one (139.00 mg; yield 70.8%; yellow oil) is used in the next step.

### Example 278

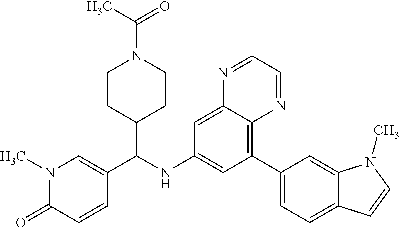 [SMILES: CC(=O)N1CCC(C(Nc2cc(-c3ccc4ccn(C)c4c3)c3nccnc3c2)c2ccc(=O)n(C)c2)CC1]

The product is prepared according to General Procedure 2 described in Example 1 with 7-chloro-5-(1-methyl-1H-indol-6-yl)-quinoxaline (Intermediate 4) (50.00 mg; 0.17 mmol; 1.00 eq.), 5-[(1-acetyl-piperidin-4-yl)-amino-methyl]-1-methyl-1H-pyridin-2-one (Intermediate 253) (92.80 mg; 0.25 mmol; 1.50 eq.), NaOtBu (56.11 mg; 0.58 mmol; 3.50 eq.), BINAP (20.77 mg; 0.03 mmol; 0.20 eq.), Pd₂(dba)₃ (15.27 mg; 0.02 mmol; 0.10 eq.) and toluene (4 mL). Purification by FCC (column deactivated with 1% TEA in DCM, then washed with DCM; DCM/MeOH; gradient). 5-{(1-Acetyl-piperidin-4-yl)-[8-(1-methyl-1H-indol-6-yl)-quinoxalin-6-ylamino]-methyl}-1-methyl-1H-pyridin-2-one (17.00 mg; yield 19.4%; 99% by HPLC) is obtained as a yellow-brown powder.

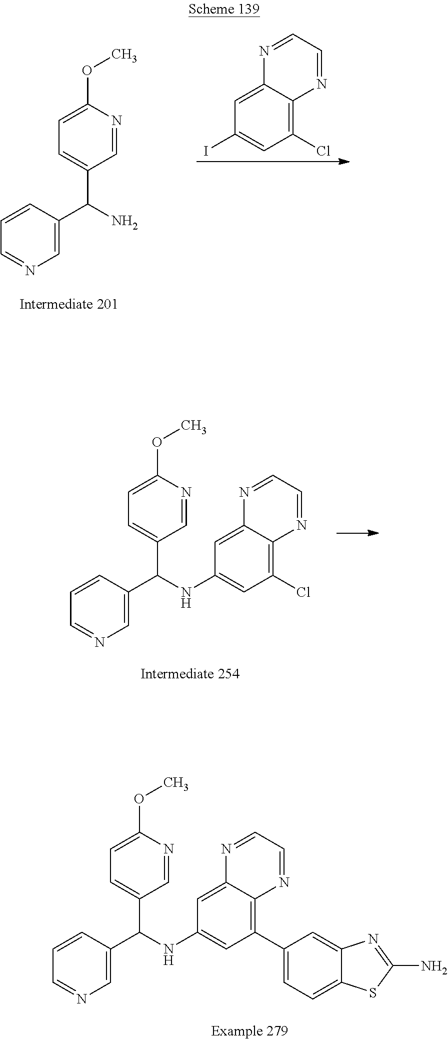 [SMILES: COc1ccc(C(N)c2cccnc2)cn1 ; Clc1cc(I)cc2nccnc12 ; COc1ccc(C(Nc2cc(Cl)c3nccnc3c2)c2cccnc2)cn1 ; COc1ccc(C(Nc2cc(-c3ccc4sc(N)nc4c3)c3nccnc3c2)c2cccnc2)cn1]

### Intermediate 254

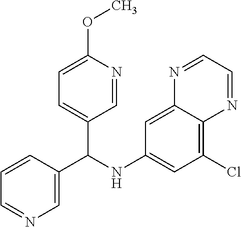 [SMILES: COc1ccc(C(Nc2cc(Cl)c3nccnc3c2)c2cccnc2)cn1]

The product is prepared according to General Procedure 2 described in Example 1 with 5-chloro-7-iodo-quinoxaline (200.00 mg; 0.69 mmol; 1.00 eq.), C-(6-methoxy-pyridin-3-yl)-C-pyridin-3-yl-methylamine (Intermediate 201) (165.54 mg; 0.72 mmol; 1.05 eq.), BINAP (85.74 mg; 0.14 mmol; 0.20 eq.), NaOtBu (165.24 mg; 1.72 mmol; 2.50 eq.), Pd₂(dba)₃ (63.05 mg; 0.07 mmol; 0.10 eq.) and toluene (5 mL). Purification by FCC (DCM/MeOH; gradient). (8-Chloro-quinoxalin-6-yl)-[(6-methoxy-pyridin-3-yl)-pyridin-3-yl-methyl]-amine (174.00 mg; yield 57.2%; 85% by UPLC) is obtained as a yellow powder.

### Example 279

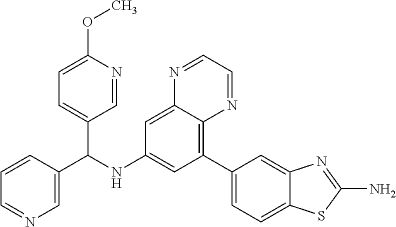 [SMILES: COc1ccc(C(Nc2cc(-c3ccc4sc(N)nc4c3)c3nccnc3c2)c2cccnc2)cn1]

The product is prepared according to General Procedure 28, described in Example 71 with (8-chloro-quinoxalin-6-yl)-[(6-methoxy-pyridin-3-yl)-pyridin-3-yl-methyl]-amine (Intermediate 254) (50.00 mg; 0.11 mmol; 1.00 eq.), 5-(4,4,5,5-tetramethyl-[1,3,2]dioxaborolan-2-yl)-benzothiazol-2-ylamine (46.34 mg; 0.13 mmol; 1.15 eq.), Na₂CO₃ (60.31 mg; 0.57 mmol; 5.00 eq.), Pd(PPh₃)₄ (13.84 mg; 0.01 mmol; 0.10 eq.), toluene (2 mL), EtOH (1 mL) and water (1 mL). Purification by FCC (DCM/MeOH; gradient; column deactivated with 1% TEA in DCM, then washed with DCM). [8-(2-Amino-benzothiazol-5-yl)-quinoxalin-6-yl]-[(6-methoxy-pyridin-3-yl)-pyridin-3-yl-methyl]-amine (30.00 mg; yield 43.9%; 91% by HPLC) is obtained as a yellow-orange powder.

### Example 280

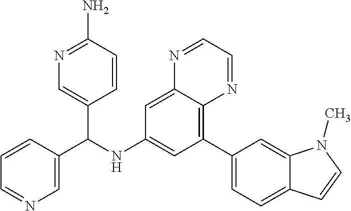 [SMILES: Cn1ccc2ccc(-c3cc(NC(c4cccnc4)c4ccc(N)nc4)cc4nccnc34)cc21]

The product is prepared according to General Procedure 2 described in Example 1 with 7-chloro-5-(1-methyl-1H-indol-6-yl)-quinoxaline (Intermediate 4) (150.00 mg; 0.51 mmol; 1.00 eq.), [5-(amino-pyridin-3-yl-methyl)-pyridin-2-yl]-carbamic acid tert-butyl ester (199.39 mg; 0.66 mmol; 1.30 eq.), NaOtBu (127.46 mg; 1.33 mmol; 2.60 eq.), BINAP (63.59 mg; 0.10 mmol; 0.20 eq.), Pd₂(dba)₃ (46.76 mg; 0.05 mmol; 0.10 eq.) and toluene (5 mL) was added. Purification by FCC (DCM/MeOH; gradient) and repurification by preparative HPLC (acidic conditions). [(6-Amino-pyridin-3-yl)-pyridin-3-yl-methyl]-[8-(1-methyl-1H-indol-6-yl)-quinoxalin-6-yl]-amine (15.00 mg; yield 6.3%; 98% by HPLC) is obtained as a yellow powder.

### Example 281

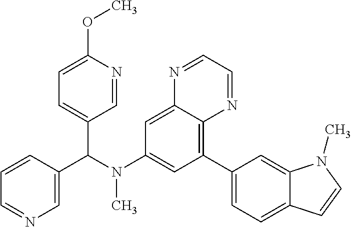 [SMILES: COc1ccc(C(c2cccnc2)N(C)c2cc(-c3ccc4ccn(C)c4c3)c3nccnc3c2)cn1]

The product is prepared according to General Procedure 40 described for Intermediate 38 with [(6-methoxy-pyridin-3-yl)-pyridin-3-yl-methyl]-[8-(1-methyl-1H-indol-6-yl)-quinoxalin-6-yl]-amine (Example 219) (50.00 mg; 0.10 mmol; 1.00 eq.), K₂CO₃ (29.01 mg; 0.21 mmol; 2.00 eq.), CH₃I (0.01 mL; 0.12 mmol; 1.10 eq.) and DMA (2 mL). Purification by FCC (DCM/MeOH; gradient). [(6-Methoxy-pyridin-3-yl)-pyridin-3-yl-methyl]-methyl-[8-(1-methyl-1H-indol-6-yl)-quinoxalin-6-yl]-amine (12.00 mg; yield 19.4%; 82% by HPLC) is obtained as a brown powder.

### Example 282

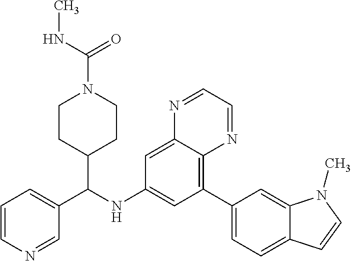 [SMILES: CNC(=O)N1CCC(C(Nc2cc(-c3ccc4ccn(C)c4c3)c3nccnc3c2)c2cccnc2)CC1]

The product is prepared according to General Procedure 35 described in Example 82 with [8-(1-methyl-1H-indol-6-yl)-quinoxalin-6-yl]-(piperidin-4-yl-pyridin-3-yl-methyl)-amine (Example 57) (50.00 mg; 0.11 mmol; 1.00 eq.), DIPEA (0.03 mL; 0.17 mmol; 1.50 eq.), N-methylcarbamoyl chloride (8.80 μL; 0.11 mmol; 1.00 eq.) and DCM (1 mL) was added. Purification by FCC (DCM/MeOH; gradient). 4-{[8-(1-Methyl-1H-indol-6-yl)-quinoxalin-6-ylamino]-pyridin-3-yl-methyl}-piperidine-1-carboxylic acid methylamide (30.00 mg; yield 53.2%; 96% by HPLC) is obtained as a yellow powder.

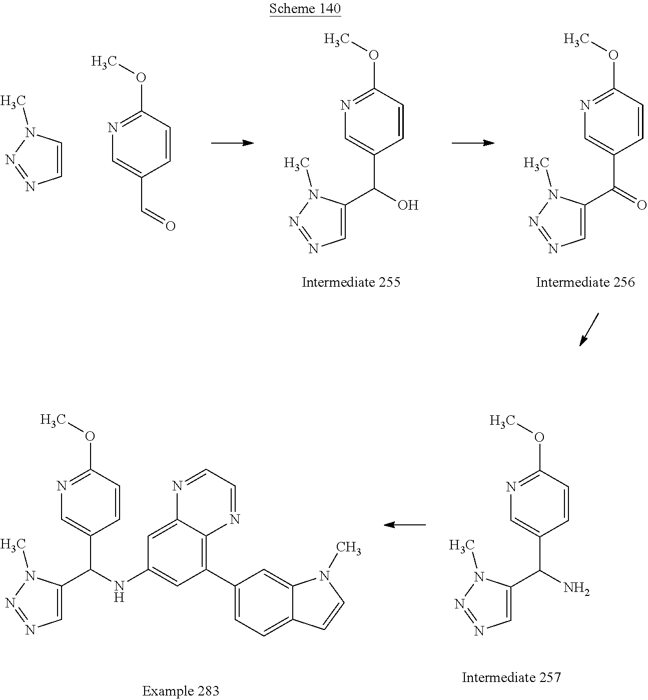

### Intermediate 255

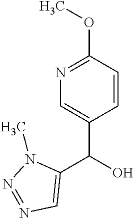 [SMILES: COc1ccc(C(O)c2cnnn2C)cn1]

The product is prepared according to General Procedure 49 described for Intermediate 75 with 1-methyl-1H-[1,2,3]triazole (213.68 μL; 3.01 mmol; 1.00 eq.), nBuLi 2.5M in hexanes (1.08 ml; 2.71 mmol; 0.90 eq.) and THF anhydrous (10 mL). Purification by FCC (DCM/MeOH: gradient). (6-Methoxy-pyridin-3-yl)-(3-methyl-3H-[1,2,3]triazol-4-yl)-methanol (334.00 mg; yield 50.4%) is obtained as a colorless oil.

### Intermediate 256

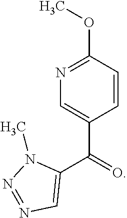 [SMILES: COc1ccc(C(=O)c2cnnn2C)cn1]

The product is prepared according to General Procedure 62 described for Intermediate 211 with (6-methoxy-pyridin-3-yl)-(3-methyl-3H-[1,2,3]triazol-4-yl)-methanol (Intermediate 255) (334.00 mg; 1.52 mmol; 1.00 eq.), MnO₂ (932.35 mg; 3.03 mmol; 2.00 eq.) and THF (5 mL). Crude product (6-methoxy-pyridin-3-yl)-(3-methyl-3H-[1,2,3]triazol-4-yl)-methanone (330.00 mg; yield 98.7%; light pink powder) is used directly in the next step without further purification.

### Intermediate 257

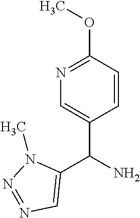 [SMILES: COc1ccc(C(N)c2cnnn2C)cn1]

The product is prepared according to General Procedure 14 described for Intermediate 12 with (6-methoxy-pyridin-3-yl)-(3-methyl-3H-[1,2,3]triazol-4-yl)-methanone (Intermediate 256) (355.00 mg; 1.63 mmol; 1.00 eq.), TTIP (0.96 mL; 3.25 mmol; 2.00 eq.), NaBH₄ (246.20 mg; 6.51 mmol; 4.00 eq.) and 7M NH₃ in MeOH (20 mL). Crude C-(6-methoxy-pyridin-3-yl)-C-(3-methyl-3H-[1,2,3]triazol-4-yl)-methylamine (303.00 mg; yield 85.0%; yellow oil) is used in the next step without further purification.

### Example 283

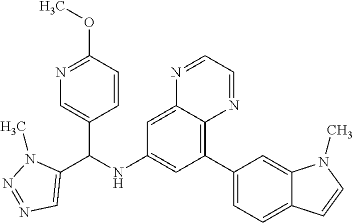 [SMILES: COc1ccc(C(Nc2cc(-c3ccc4ccn(C)c4c3)c3nccnc3c2)c2cnnn2C)cn1]

The product is prepared according to General Procedure 2 described in Example 1 with C-(6-methoxy-pyridin-3-yl)-C-(3-methyl-3H-[1,2,3]triazol-4-yl)-methylamine (Intermediate 257) (111.95 mg; 0.51 mmol; 1.50 eq.), 7-chloro-5-(1-methyl-1H-indol-6-yl)-quinoxaline (intermediate 4) (100.00 mg; 0.34 mmol; 1.00 eq.), NaOtBu (65.43 mg; 0.68 mmol; 2.00 eq.), Pd₂(dba)₃ (31.17 mg; 0.03 mmol; 0.10 eq.), BINAP (42.39 mg; 0.07 mmol; 0.20 eq.) and toluene (5 mL). Purification by FCC (DCM:MeOH; gradient). [(6-Methoxy-pyridin-3-yl)-(3-methyl-3H-[1,2,3]triazol-4-yl)-methyl]-[8-(1-methyl-1H-indol-6-yl)-quinoxalin-6-yl]-amine (70.00 mg; yield 41.6%; 96% by HPLC) is obtained as a yellow powder.

### Example 284

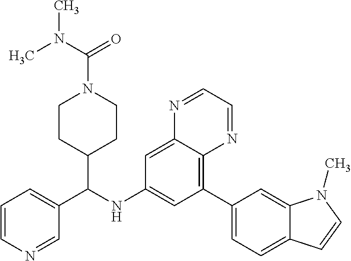 [SMILES: CN(C)C(=O)N1CCC(C(Nc2cc(-c3ccc4ccn(C)c4c3)c3nccnc3c2)c2cccnc2)CC1]

The product is prepared according to General Procedure 35 described in Example 82 with [8-(1-methyl-1H-indol-6-yl)-quinoxalin-6-yl]-(piperidin-4-yl-pyridin-3-yl-methyl)-amine (Example 57) (50.00 mg; 0.11 mmol; 1.00 eq.), N,N-dimethylcarbamoyl chloride (11.99 mg; 0.11 mmol; 1.00 eq.) and DCM (5 mL). Purification by FCC (DCM/MeOH; gradient). 4-{[8-(1-Methyl-1H-indol-6-yl)-quinoxalin-6-ylamino]-pyridin-3-yl-methyl}-piperidine-1-carboxylic acid dimethylamide (27.00 mg; yield 44.7%; 96% by HPLC) is obtained as a yellow powder.

### Example 285

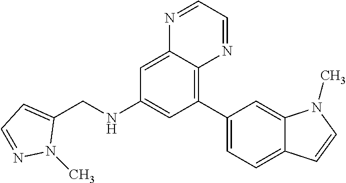 [SMILES: Cn1nccc1CNc1cc(-c2ccc3ccn(C)c3c2)c2nccnc2c1]

The product is prepared according to General Procedure 2 described in Example 1 with 7-chloro-5-(1-methyl-1H-indol-6-yl)-quinoxaline (Intermediate 4) (67.00 mg; 0.6 mmol; 2.00 eq.), C-(2-methyl-2H-pyrazol-3-yl)-methylamine (66.65 mg; 0.58 mmol; 2.00 eq.), NaOtBu (83.43 mg; 0.87 mmol; 3.00 eq.), BINAP (36.04 mg; 0.06 mmol; 0.20 eq.), Pd₂(dba)₃ (26.50 mg; 0.03 mmol; 0.10 eq.) and toluene (3 mL). Purification by FCC (hexane/EtOAc; gradient). [8-(1-Methyl-1H-indol-6-yl)-quinoxalin-6-yl]-(2-methyl-2H-pyrazol-3-ylmethyl)-amine (74.00 mg; yield 66.8%; 96% by HPLC) is obtained as a yellow powder.

### Example 286

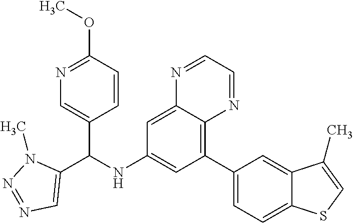 [SMILES: COc1ccc(C(Nc2cc(-c3ccc4scc(C)c4c3)c3nccnc3c2)c2cnnn2C)cn1]

The product is prepared according to General Procedure 2 described in Example 1 with 7-chloro-5-(3-methyl-benzo[b]thiophen-5-yl)-quinoxaline (Intermediate 161) (70.00 mg; 0.23 mmol; 1.00 eq.), C-(6-methoxy-pyridin-3-yl)-C-(3-methyl-3H-[1,2,3]triazol-4-yl)-methylamine (Intermediate 257) (98.76 mg; 0.45 mmol; 2.00 eq.), NaOtBu (86.58 mg; 0.90 mmol; 4.00 eq.), Pd₂(dba)₃ (21.71 mg; 0.02 mmol; 0.10 eq.), BINAP (28.05 mg; 0.05 mmol; 0.20 eq.) and toluene (2 mL). Purification by FCC (hexane/EtOAc; gradient and then EtOAc/MeOH; gradient). [(6-Methoxy-pyridin-3-yl)-(3-methyl-3H-[1,2,3]triazol-4-yl)-methyl]-[8-(3-methyl-benzo[b]thiophen-5-yl)-quinoxalin-6-yl]-amine (50.00 mg; yield 43.7%; 97% by HPLC) is obtained as a yellow powder.

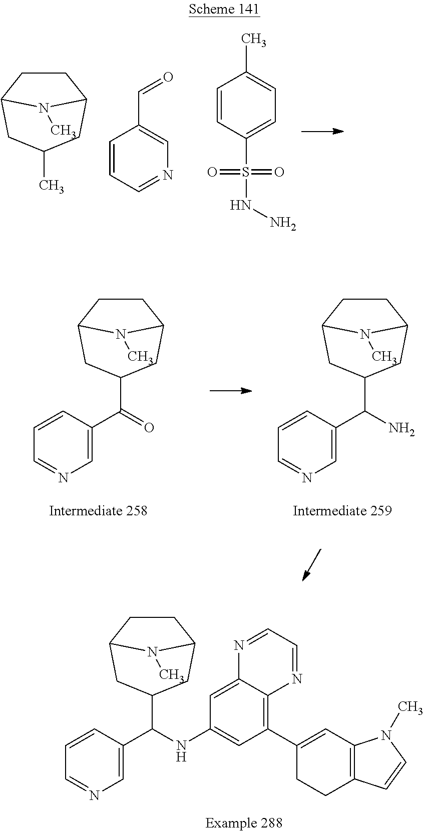

### Intermediate 258

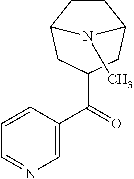 [SMILES: CN1C2CCC1CC(C(=O)c1cccnc1)C2]

The product is prepared according to General Procedure 12 described for Intermediate 10 with 8-methyl-8-aza-bicyclo[3.2.1]octan-3-one (300.00 mg; 2.16 mmol; 1.00 eq.), pyridine-3-carbaldehyde (230.85 mg; 2.16 mmol; 1.00 eq.), 4-methylbenzenesulfonohydrazide (401.38 mg; 2.16 mmol; 1.00 eq.), Cs₂CO₃ (1053.34 mg; 3.23 mmol; 1.50 eq.), MeOH (5 mL) and 1,4-dioxane (5 mL). Purification by FCC (NH₂ column; DCM/MeOH; gradient). (8-Methyl-8-aza-bicyclo[3.2.1]oct-3-yl)-pyridin-3-yl-methanone (203.00 mg; yield 33.0%; 80% by UPLC) is obtained as a yellow oil.

### Intermediate 259

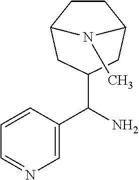 [SMILES: CN1C2CCC1CC(C(N)c1cccnc1)C2]

The product is prepared according to General Procedure 14 described for Intermediate 12 with (8-methyl-8-aza-bicyclo[3.2.1]oct-3-yl)-pyridin-3-yl-methanone (Intermediate 259) (203.00 mg; 0.71 mmol; 1.00 eq.), TTIP (0.42 mL; 1.43 mmol; 2.00 eq.), NaBH₄ (108.05 mg; 2.86 mmol; 4.00 eq.) and 7M NH₃ in MeOH (4 mL). Crude C-(8-methyl-8-aza-bicyclo[3.2.1]oct-3-yl)-C-pyridin-3-yl-methylamine (164.00 mg; yield 80.4%; yellow oil) is used in the next step.

### Example 288

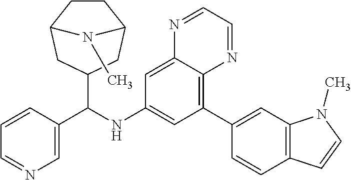 [SMILES: CN1C2CCC1CC(C(Nc1cc(-c3ccc4ccn(C)c4c3)c3nccnc3c1)c1cccnc1)C2]

The product is prepared according to General Procedure 2 described in Example 1 with 7-chloro-5-(1-methyl-1H-indol-6-yl)-quinoxaline (Intermediate 4) (16.00 mg; 0.05 mmol; 1.00 eq.), C-(8-methyl-8-aza-bicyclo[3.2.1]oct-3-yl)-C-pyridin-3-yl-methylamine (22.87 mg; 0.08 mmol; 1.50 eq.), NaOtBu (17.95 mg; 0.19 mmol; 3.50 eq.), BINAP (6.65 mg; 0.01 mmol; 0.20 eq.), Pd₂(dba)₃ (4.89 mg; 0.01 mmol; 0.10 eq.) and toluene (4 mL). Purification by FCC (Puriflash DIOL 50 UM column; DCM/MeOH; gradient). [(8-Methyl-8-aza-bicyclo[3.2.1]oct-3-yl)-pyridin-3-yl-methyl]-[8-(1-methyl-1H-indol-6-yl)-quinoxalin-6-yl]-amine (15.00 mg; yield 50.0%; 87% by HPLC) is obtained as a yellow powder.

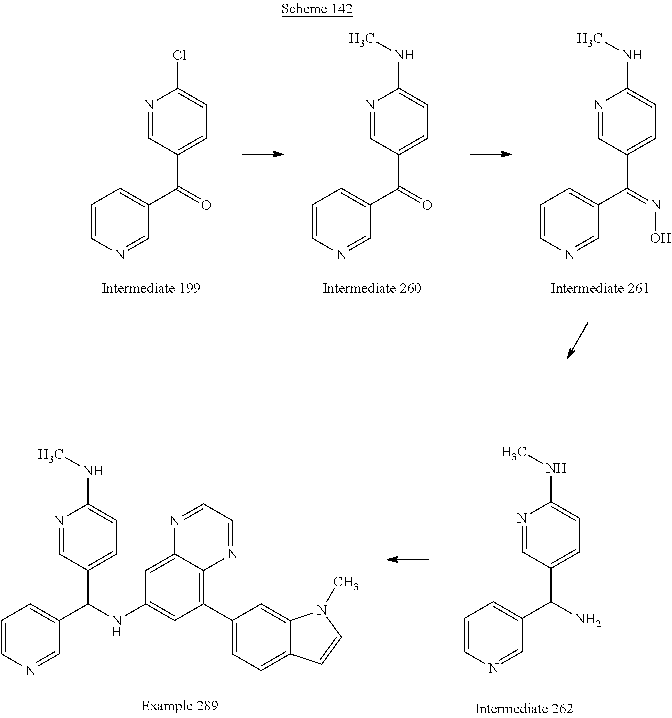 [SMILES: O=C(c1cccnc1)c1ccc(Cl)nc1 ; CNc1ccc(C(=O)c2cccnc2)cn1 ; CNc1ccc(/C(=N/O)c2cccnc2)cn1 ; CNc1ccc(C(Nc2cc(-c3ccc4ccn(C)c4c3)c3nccnc3c2)c2cccnc2)cn1 ; CNc1ccc(C(N)c2cccnc2)cn1]

### Intermediate 260

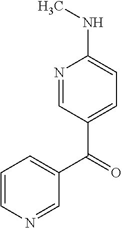 [SMILES: CNc1ccc(C(=O)c2cccnc2)cn1]

Mixture of (6-chloro-pyridin-3-yl)-pyridin-3-yl-methanone (Intermediate 199) (500.00 mg; 2.26 mmol; 1.00 eq.), methylamine 40% solution in water (1 mL; 11.62 mmol; 5.13 eq.) in DMSO (0.5 mL) is stirred at 40° C. for 2 hours. Extraction with mixture of DCM:iPrOH (4:1). Purification by FCC (DCM/MeOH; gradient). (6-Methylamino-pyridin-3-yl)-pyridin-3-yl-methanone (255.00 mg; yield 52.3%) is obtained as a yellow powder.

### Intermediate 261

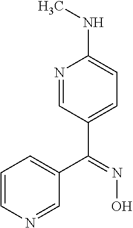 [SMILES: CNc1ccc(/C(=N/O)c2cccnc2)cn1]

The product is prepared according to General Procedure 17 described for Intermediate 19 with (6-methylamino-pyridin-3-yl)-pyridin-3-yl-methanone (Intermediate 260) (225.00 mg; 1.06 mmol; 1.00 eq.), NaOAc (216.40 mg; 2.64 mmol; 2.50 eq.), hydroxylamine hydrochloride (183.31 mg; 2.64 mmol; 2.50 eq.) and MeOH anhydrous (10 mL). Crude (6-methylamino-pyridin-3-yl)-pyridin-3-yl-methanone oxime (240.00 mg; yield 98.7%; pale yellow gum) is used in the next step.

### Intermediate 262

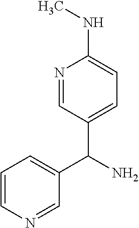 [SMILES: CNc1ccc(C(N)c2cccnc2)cn1]

The product is prepared according to General Procedure 18 described for Intermediate 20 with (6-methylamino-pyridin-3-yl)-pyridin-3-yl-methanone oxime (Intermediate 261) (250.00 mg; 1.10 mmol; 1.00 eq.), NH₄OAc (126.64 mg; 1.64 mmol; 1.50 eq.), ammonia 25% (3 mL), zinc dust (0.36 g; 5.48 mmol; 5.00 eq.), EtOH (3 mL) and water (3 mL). Crude [5-(amino-pyridin-3-yl-methyl)-pyridin-2-yl]-methyl-amine (190.00 mg; yield 79.3%; gummy oil) is used in the next step.

### Example 289

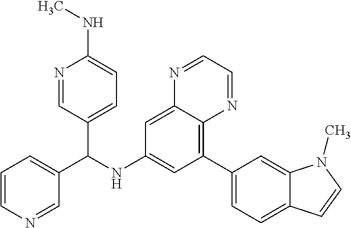 [SMILES: CNc1ccc(C(Nc2cc(-c3ccc4ccn(C)c4c3)c3nccnc3c2)c2cccnc2)cn1]

The product is prepared according to General Procedure 5 described in Example 30 with 7-chloro-5-(1-methyl-1H-indol-6-yl)-quinoxaline (Intermediate 4) (150.00 mg; 0.51 mmol; 1.00 eq.), [5-(amino-pyridin-3-yl-methyl)-pyridin-2-yl]-methyl-amine (Intermediate 262) (131.30 mg; 0.61 mmol; 1.20 eq.), NaOtBu (58.89 mg; 0.61 mmol; 1.20 eq.), BippyPhos (12.93 mg; 0.03 mmol; 0.05 eq.), [(Cinnamyl)PdCl]₂ (3.31 mg; 0.01 mmol; 0.01 eq.) and toluene (0.50 mL). Purification by FCC (Al₂O₃; DCM/MeOH; gradient). Repurification by FCC (DCM/MeOH; gradient). [(6-Methylamino-pyridin-3-yl)-pyridin-3-yl-methyl]-[8-(1-methyl-1H-indol-6-yl)-quinoxalin-6-yl]-amine (48.90 mg; yield 18.4%; 90% by HPLC) is obtained as a pale brown powder.

### Example 290

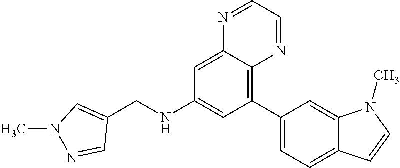 [SMILES: Cn1cc(CNc2cc(-c3ccc4ccn(C)c4c3)c3nccnc3c2)cn1]

The product is prepared according to General Procedure 5 described in Example 30 with 7-chloro-5-(1-methyl-1H-indol-6-yl)-quinoxaline (Intermediate 4) (90.000 mg; 0.28 mmol; 1.00 eq.), C-(1-methyl-1H-pyrazol-4-yl)-methylamine (61.908 mg; 0.56 mmol; 2.00 eq.), BippyPhos (11.287 mg; 0.02 mmol; 0.08 eq.), NaOtBu (80.292 mg; 0.84 mmol; 3.00 eq.), [(Cinnamyl)PdCl]₂ (7.214 mg; 0.01 mmol; 0.05 eq.) and toluene anhydrous (1.5 mL). Purification by FCC (DCM/EtOAc; gradient and then EtOAc/MeOH; gradient). [8-(1-Methyl-1H-indol-6-yl)-quinoxalin-6-yl]-(1-methyl-1H-pyrazol-4-ylmethyl)-amine (30.10 mg; yield 27.7%; 94% by HPLC) is obtained as a yellow solid.

### Example 291

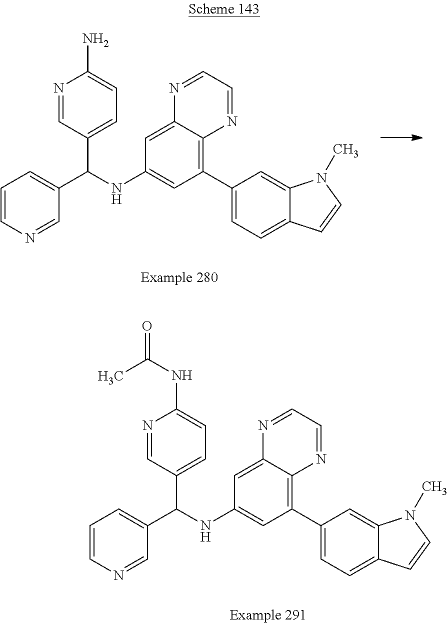 [SMILES: Cn1ccc2ccc(-c3cc(NC(c4cccnc4)c4ccc(N)nc4)cc4nccnc34)cc21 ; CC(=O)Nc1ccc(C(Nc2cc(-c3ccc4ccn(C)c4c3)c3nccnc3c2)c2cccnc2)cn1]

The product is prepared according to General Procedure 65 described for Intermediate 222 with [(6-amino-pyridin-3-yl)-pyridin-3-yl-methyl]-[8-(1-methyl-1H-indol-6-yl)-quinoxalin-6-yl]-amine (Example 280) (30.00 mg; 0.07 mmol; 1.00 eq.), DCC (14.88 mg; 0.07 mmol; 1.10 eq.), CH₃COOH (4.13 μL; 0.07 mmol; 1.10 eq.) and DCM anhydrous (3 mL). Purification by FCC (DCM/MeOH; gradient). N-(5-{[8-(1-Methyl-1H-indol-6-yl)-quinoxalin-6-ylamino]-pyridin-3-yl-methyl}-pyridin-2-yl)-acetamide (2.50 mg; yield 6.9%; 91% by HPLC) is obtained as a yellow powder.

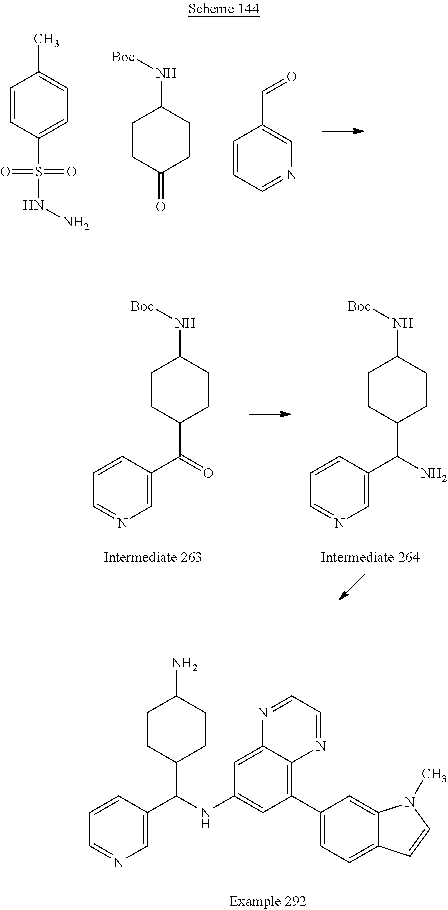

### Intermediate 263

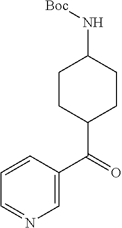 [SMILES: CC(C)(C)OC(=O)NC1CCC(C(=O)c2cccnc2)CC1]

The product is prepared according to General Procedure 15 described for Intermediate 13 with (4-oxo-cyclohexyl)-carbamic acid tert-butyl ester (400.00 mg; 1.88 mmol; 1.00 eq.), pyridine-3-carbaldehyde (0.18 mL; 1.88 mmol; 1.00 eq.), Cs₂CO₃ (458.31 mg; 1.41 mmol; 0.75 eq.), 4-methylbenzenesulfonohydrazide (349.28 mg; 1.88 mmol; 1.00 eq.), MeOH (20 mL) and 1,4-dioxane (20 mL). Purification by FCC (DCM/MeOH; gradient). [4-(Pyridine-3-carbonyl)-cyclohexyl]-carbamic acid tert-butyl ester (350.00 mg; yield 61.3%) is obtained as a yellow oil.

### Intermediate 264

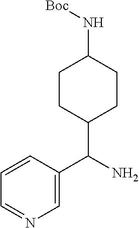 [SMILES: CC(C)(C)OC(=O)NC1CCC(C(N)c2cccnc2)CC1]

The product is prepared according to General Procedure 14 described for Intermediate 12 with [4-(pyridine-3-carbonyl)-cyclohexyl]-carbamic acid tert-butyl ester (Intermediate 263) (350.00 mg; 1.15 mmol; 1.00 eq.), TTIP (0.68 mL; 2.30 mmol; 2.00 eq.), NaBH₄ (174.01 mg; 4.60 mmol; 4.00 eq.) and 7M NH₃ in MeOH (20 mL). Crude [4-(amino-pyridin-3-yl-methyl)-cyclohexyl]-carbamic acid tert-butyl ester (430.00 mg; yield 39.2%; yellow oil) is used in the next step.

### Example 292

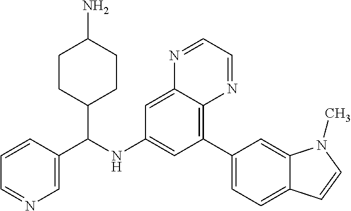 [SMILES: Cn1ccc2ccc(-c3cc(NC(c4cccnc4)C4CCC(N)CC4)cc4nccnc34)cc21]

The product is prepared according to General Procedure 2 described in Example 1 with 7-chloro-5-(1-methyl-1H-indol-6-yl)-quinoxaline (Intermediate 4) (150.00 mg; 0.51 mmol; 1.00 eq.), [4-(amino-pyridin-3-yl-methyl)-cyclohexyl]-carbamic acid tert-butyl ester (Intermediate 264) (233.93 mg; 0.77 mmol; 1.50 eq.), NaOtBu (171.76 mg; 1.79 mmol; 3.50 eq.), BINAP (63.59 mg; 0.10 mmol; 0.20 eq.), Pd₂(dba)₃ (46.76 mg; 0.05 mmol; 0.10 eq.) and toluene (10 mL). Purification by FCC (DCM/MeOH: gradient). Repurification by preparative HPLC (acidic conditions). [(4-Amino-cyclohexyl)-pyridin-3-yl-methyl]-[8-(1-methyl-1H-indol-6-yl)-quinoxalin-6-yl]-amine (30.00 mg; yiled 12.7%; 98% by HPLC) is obtained as a yellow powder.

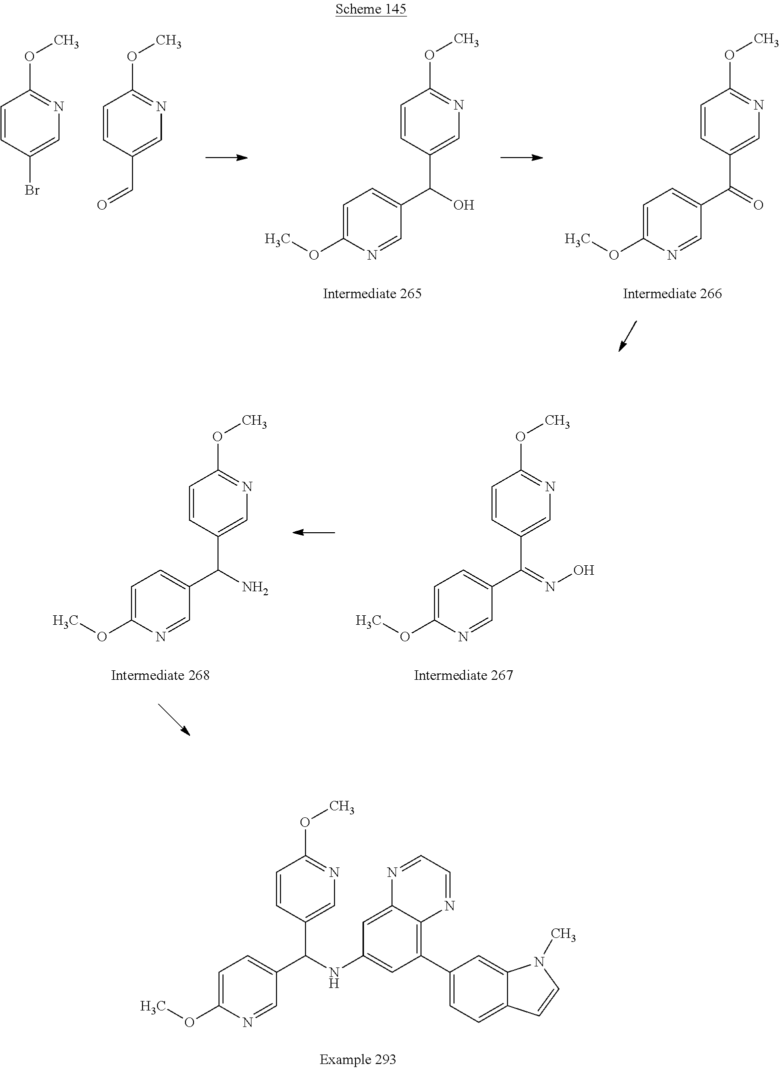

### Intermediate 265

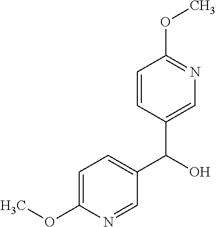

The product is prepared according to General Procedure 60 described for Intermediate 198 with 5-bromo-2-methoxy-pyridine (4.18 mL; 36.46 mmol; 2.50 eq.), 6-methoxy-pyridine-3-carbaldehyde (2.00 g; 14.58 mmol; 1.00 eq.), isopropylmagnesium chloride/LiCl solution 1.3M in THF (28.05 mL; 36.46 mmol; 2.50 eq.) and anhydrous THF (45 mL). Purification by FCC (DCM/MeOH; gradient). Bis-(6-methoxy-pyridin-3-yl)-methanol (1.93 g; yield 44.1%) is obtained as a bright brown oil.

### Intermediate 266

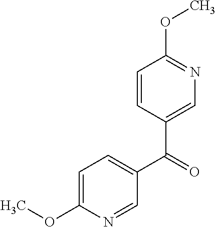

The product is prepared according to General Procedure 62 described for Intermediate 211 with bis-(6-methoxy-pyridin-3-yl)-methanol (Intermediate 265) (1.93 g; 6.43 mmol; 1.00 eq.), MnO₂ (3.95 g; 12.85 mmol; 2.00 eq.) and THF (10 mL). Crude bis-(6-methoxy-pyridin-3-yl)-methanone (1.8 g; yield 96%, 84% by UPLC) is used in the next step.

### Intermediate 267

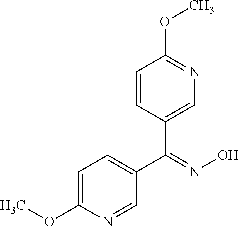 [SMILES: COc1ccc(C(=NO)c2ccc(OC)nc2)cn1]

The product is prepared according to General Procedure 17 described for Intermediate 19 with bis-(6-methoxy-pyridin-3-yl)-methanone (Intermediate 266) (1.80 g; 6.19 mmol; 1.00 eq.), NaOAc (1.21 g; 14.72 mmol; 2.50 eq.), hydroxylamine hydrochloride (1.02 g; 14.72 mmol; 2.50 eq.) and MeOH (30 mL). Purification by FCC (DCM). Bis-(6-methoxy-pyridin-3-yl)-methanone oxime (1.41 g; yield 87.9%) is obtained as a colorless powder.

### Intermediate 268

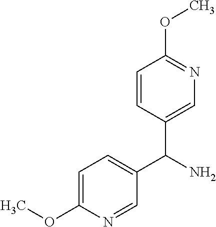

The product is prepared according to General Procedure 18 described for Intermediate 20 with bis-(6-methoxy-pyridin-3-yl)-methanone oxime (Intermediate 267) (1.40 g; 5.40 mmol; 1.00 eq.), NH₄OAc (624.34 mg; 8.10 mmol; 1.50 eq.), zinc dust (1.77 g; 27.00 mmol; 5.00 eq.), ammonia 25% (9 mL), EtOH (9 mL) and water (9 mL). Crude C,C-bis-(6-methoxy-pyridin-3-yl)-methylamine (1.0 g; yield 75%, 98% by UPLC) is used in the next step.

### Example 293

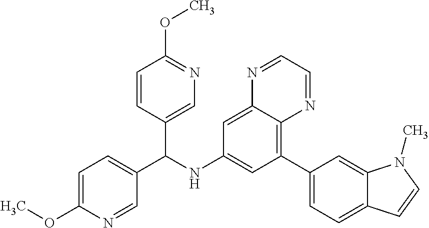 [SMILES: COc1ccc(C(Nc2cc(-c3ccc4ccn(C)c4c3)c3nccnc3c2)c2ccc(OC)nc2)cn1]

The product is prepared according to General Procedure 2 described in Example 1 with 7-chloro-5-(1-methyl-1H-indol-6-yl)-quinoxaline (Intermediate 4) (150.00 mg; 0.47 mmol; 1.00 eq.), C,C-bis-(6-methoxy-pyridin-3-yl)-methylamine (Intermediate 268) (233.37 mg; 0.93 mmol; 2.00 eq.), NaOtBu (179.22 mg; 1.86 mmol; 4.00 eq.), Bis(tri-tert-butylphosphine)palladium(0) (23.83 mg; 0.05 mmol; 0.10 eq.) and 1,4-dioxane (2 mL). Purification by FCC (DCM/MeOH; gradient). [Bis-(6-methoxy-pyridin-3-yl)-methyl]-[8-(1-methyl-1H-indol-6-yl)-quinoxalin-6-yl]-amine (168.00 mg; yield 67.6%; 94% by HPLC) is obtained as a yellow solid.

### Example 294 & Example 295

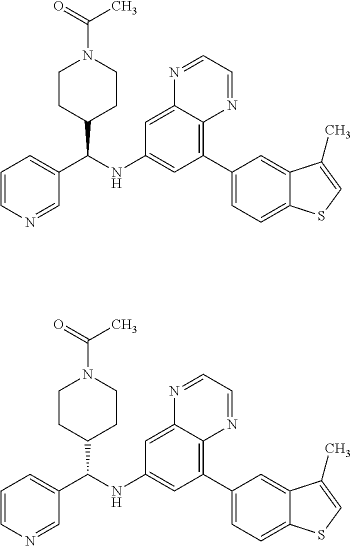 [SMILES: CC(=O)N1CCC([C@@H](Nc2cc(-c3ccc4scc(C)c4c3)c3nccnc3c2)c2cccnc2)CC1 ; CC(=O)N1CCC([C@H](Nc2cc(-c3ccc4scc(C)c4c3)c3nccnc3c2)c2cccnc2)CC1]

1-(4-{[8-(3-Methyl-benzo[b]thiophen-5-yl)-quinoxalin-6-ylamino]-pyridin-3-yl-methyl}-piperidin-1-yl)-ethanone (Example 254) (90.00 mg; 0.17 mmol; 1.00 eq.) is dissolved in isopropyl alcohol and compound is separated by HPLC (HPLC with UV-Vis or DAD detector; column: Chiralpak AYH; (A)EtOH +0.1% DEA, (B)HEXAN +0.1% DEA, gradient 60% (B). Both enantiomers: 1-(4-{(R)-[8-(3-methyl-benzo[b]thiophen-5-yl)-quinoxalin-6-ylamino]-pyridin-3-yl-methyl}-piperidin-1-yl)-ethanone (Example 294) (20.50 mg; yield 22.9%; yellow powder) and 1-(4-{(S)-[8-(3-methyl-benzo[b]thiophen-5-yl)-quinoxalin-6-ylamino]-pyridin-3-yl-methyl}-piperidin-1-yl)-ethanone (Example 295) (28.00 mg; yield 30.6%; orange powder) are isolated with 99% of optical purity.

### Example 296

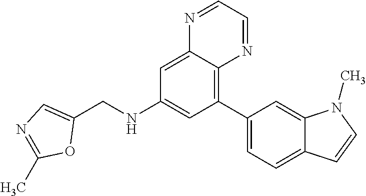 [SMILES: Cc1ncc(CNc2cc(-c3ccc4ccn(C)c4c3)c3nccnc3c2)o1]

The product is prepared according to General Procedure 23 described in Example 63 with 8-(1-methyl-1H-indol-6-yl)-quinoxalin-6-ylamine (Intermediate 22) (80.00 mg; 0.29 mmol; 1.00 eq.), 2-methyl-oxazole-5-carbaldehyde (31.75 mg; 0.29 mmol; 1.00 eq.), Hantzsch ester (90.49 mg; 0.36 mmol; 1.25 eq.), TMCS (7.25 μL; 0.06 mmol; 0.20 eq.) and DCM anhydrous (3 mL). Purification by FCC (DCM/MeOH; gradient). [8-(1-Methyl-1H-indol-6-yl)-quinoxalin-6-yl]-(2-methyl-oxazol-5-ylmethyl)-amine (63.00 mg; yield 59.4%; 99% by HPLC) is obtained as a yellow powder.

### Example 297

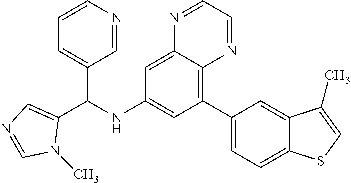 [SMILES: Cc1csc2ccc(-c3cc(NC(c4cccnc4)c4cncn4C)cc4nccnc34)cc12]

The product is prepared according to General Procedure 2 described in Example 63 with 7-chloro-5-(3-methyl-benzo[b]thiophen-5-yl)-quinoxaline (Intermediate 161) (60.00 mg; 0.19 mmol; 1.00 eq.), C-(3-methyl-3H-imidazol-4-yl)-C-pyridin-3-yl-methylamine (Intermediate 226) (62.33 mg; 0.28 mmol; 1.50 eq.), NaOtBu (54.49 mg; 0.57 mmol; 3.00 eq.), BINAP (11.78 mg; 0.02 mmol; 0.10 eq.), Pd₂(dba)₃ (8.66 mg; 0.01 mmol; 0.05 eq.) and toluene (4 mL). Purification by FCC (DCM/MeOH; gradient; column deactivated with 1% Et₃N in DCM and washed with DCM). [8-(3-Methyl-benzo[b]thiophen-5-yl)-quinoxalin-6-yl]-[(3-methyl-3H-imidazol-4-yl)-pyridin-3-yl-methyl]-amine (18.50 mg; yield 19.7%; 93% by HPLC) is obtained as a yellow-brown powder.

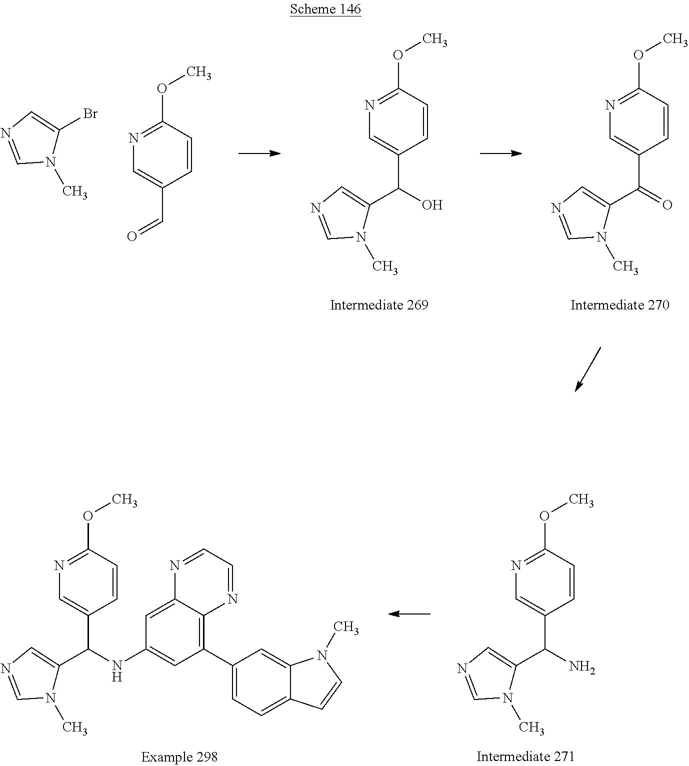

### Intermediate 269

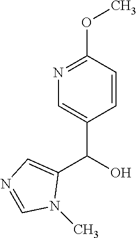 [SMILES: COc1ccc(C(O)c2cncn2C)cn1]

The product is prepared according to General Procedure 60 described for Intermediate 198 with 5-bromo-1-methyl-1H-imidazole (587.01 mg; 3.65 mmol; 2.50 eq.), 6-methoxy-pyridine-3-carbaldehyde (200.00 mg; 1.46 mmol; 1.00 eq.), isopropylmagnesium chloride/LiCl solution 1.3 M in THF (2.80 mL; 3.65 mmol; 2.50 eq.) and THF anhydrous (5 mL). Purification by FCC (DCM/MeOH; gradient). (6-Methoxy-pyridin-3-yl)-(3-methyl-3H-imidazol-4-yl)-methanol (158.00 mg; yield 49.4%; 100% by UPLC) is obtained as a yellow oil.

### Intermediate 270

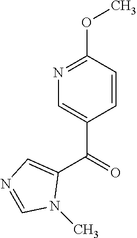 [SMILES: COc1ccc(C(=O)c2cncn2C)cn1]

The product is prepared according to General Procedure 62 described for Intermediate 221 with (6-methoxy-pyridin-3-yl)-(3-methyl-3H-imidazol-4-yl)-methanol (Intermediate 269) (250.00 mg; 0.73 mmol; 1.00 eq.), MnO₂ (897.30 mg; 2.92 mmol; 4.00 eq.) and THF anhydrous (6 mL). Crude (6-methoxy-pyridin-3-yl)-(3-methyl-3H-imidazol-4-yl)-methanone (228.00 mg; yield 130.9%; white powder) is used in the next step without further purification.

### Intermediate 271

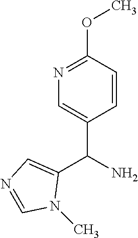 [SMILES: COc1ccc(C(N)c2cncn2C)cn1]

The product is prepared according to General Procedure 14 described for Intermediate 12 with (6-methoxy-pyridin-3-yl)-(3-methyl-3H-imidazol-4-yl)-methanone (Intermediate 270) (150.00 mg; 0.63 mmol; 1.00 eq.), TTIP (0.37 mL; 1.26 mmol; 2.00 eq.), NaBH₄ (95.09 mg; 2.51 mmol; 4.00 eq.) and 7M NH₃ in MeOH (4 mL). Crude C-(6-methoxy-pyridin-3-yl)-C-(3-methyl-3H-imidazol-4-yl)-methylamine (150.00 mg; yield 97.7%; 89% by UPLC; yellow oil) is used in the next step.

### Example 298

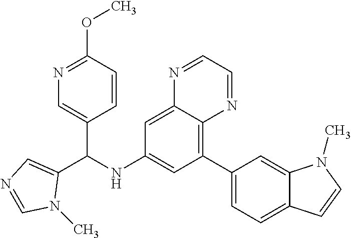 [SMILES: COc1ccc(C(Nc2cc(-c3ccc4ccn(C)c4c3)c3nccnc3c2)c2cncn2C)cn1]

The product is prepared according to General Procedure 6 described for Intermediate 6 with 7-chloro-5-(1-methyl-1H-indol-6-yl)-quinoxaline (Intermediate 4) (55.00 mg; 0.18 mmol; 1.00 eq.), C-(6-methoxy-pyridin-3-yl)-C-(3-methyl-3H-imidazol-4-yl)-methylamine (Intermediate 271) (89.69 mg; 0.37 mmol; 2.00 eq.), Cs₂CO₃ (181.17 mg; 0.55 mmol; 3.00 eq.), BINAP (11.66 mg; 0.02 mmol; 0.10 eq.), Pd(OAc)₂ (4.34 mg; 0.02 mmol; 0.10 eq.) and 1,4-dioxane (3 mL). Purification by FCC (DCM/MeOH; gradient). [(6-Methoxy-pyridin-3-yl)-(3-methyl-3H-imidazol-4-yl)-methyl]-[8-(1-methyl-1H-indol-6-yl)-quinoxalin-6-yl]-amine (59.00 mg; yield 67.1%; 99% by HPLC) is obtained as a yellow solid.

### Example 299

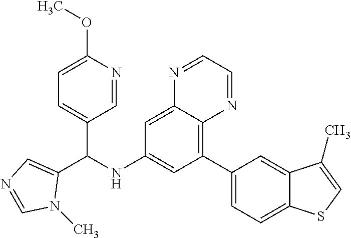 [SMILES: COc1ccc(C(Nc2cc(-c3ccc4scc(C)c4c3)c3nccnc3c2)c2cncn2C)cn1]

The product is prepared according to General Procedure 2 described in Example 1 with 7-chloro-5-(3-methyl-benzo[b]thiophen-5-yl)-quinoxaline (Intermediate 161) (70.00 mg; 0.23 mmol; 1.00 eq.), C-(6-methoxy-pyridin-3-yl)-C-(3-methyl-3H-imidazol-4-yl)-methylamine (Intermediate 271) (98.31 mg; 0.45 mmol; 2.00 eq.), NaOtBu (86.49 mg; 0.90 mmol; 4.00 eq.), Pd₂(dba)₃ (20.62 mg; 0.02 mmol; 0.10 eq.), BINAP (28.05 mg; 0.05 mmol; 0.20 eq.) and toluene (5 mL). Purification by FCC (DCM/MeOH; gradient). [(6-Methoxy-pyridin-3-yl)-(3-methyl-3H-imidazol-4-yl)-methyl]-[8-(3-methyl-benzo[b]thiophen-5-yl)-quinoxalin-6-yl]-amine (85.00 mg; yield 74.4%; 97% by HPLC) is obtained as a brown powder.

### Example 300

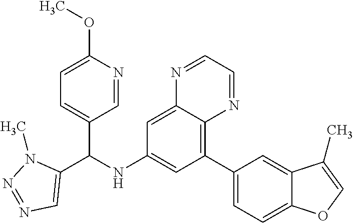 [SMILES: COc1ccc(C(Nc2cc(-c3ccc4occ(C)c4c3)c3nccnc3c2)c2cnnn2C)cn1]

The product is prepared according to General Procedure 2 described in Example 1 with 7-chloro-5-(3-methyl-benzofuran-5-yl)-quinoxaline (Intermediate 60) (70.00 mg; 0.24 mmol; 1.00 eq.), C-(6-methoxy-pyridin-3-yl)-C-(3-methyl-3H-[1,2,3]triazol-4-yl)-methylamine (Intermediate 257) (93.73 mg; 0.43 mmol; 1.80 eq.), NaOtBu (68.47 mg; 0.71 mmol; 3.00 eq.), BINAP (14.79 mg; 0.02 mmol; 0.10 eq.), Pd₂(dba)₃ (21.75 mg; 0.02 mmol; 0.10 eq.) and toluene (3 mL). Purification by FCC (DCM/MeOH; gradient). [(6-Meth-oxy-pyridin-3-yl)-(3-methyl-3H-[1,2,3]triazol-4-yl)-methyl]-[8-(3-methyl-benzofuran-5-yl)-quinoxalin-6-yl]-amine (63.00 mg; yield 53.8%; 96% by HPLC) is obtained as a brown powder.

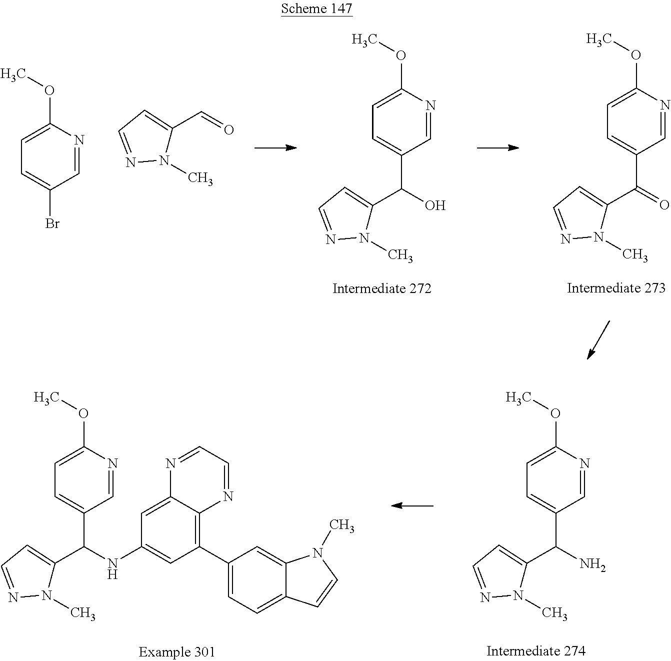

### Intermediate 272

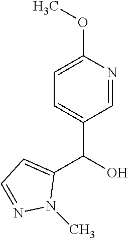 [SMILES: COc1ccc(C(O)c2ccnn2C)cn1]

The product is prepared according to General Procedure 60 described for Intermediate 198 with 5-bromo-2-methoxy-pyridine (0.45 mL; 3.63 mmol; 2.00 eq.), 2-methyl-2H-pyrazole-3-carbaldehyde (0.18 mL; 1.82 mmol; 1.00 eq.), isopropylmagnesium chloride/LiCl solution 1.3 M in THF (2.79 mL; 3.63 mmol; 2.00 eq.) and anhydrous THF (15 mL). Crude (6-methoxy-pyridin-3-yl)-(2-methyl-2H-pyrazol-3-yl)-methanol (700.00 mg; yield 78.1%; light yellow oil) is used in the next step without further purification.

### Intermediate 273

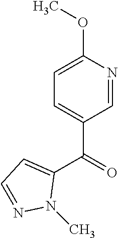 [SMILES: COc1ccc(C(=O)c2ccnn2C)cn1]

The product is prepared according to General Procedure 62 described for Intermediate 211 with (6-methoxy-pyridin-3-yl)-(2-methyl-2H-pyrazol-3-yl)-methanol (Intermediate 272) (700.00 mg; 3.19 mmol; 1.00 eq.), MnO₂ (1962.84 mg; 6.39 mmol; 2.00 eq.) and THF (5 mL). Purification by FCC (DCM/MeOH; gradient). (6-Methoxy-pyridin-3-yl)-(2-methyl-2H-pyrazol-3-yl)-methanone (420.00 mg; yield 60.2%) is obtained as a light yellow oil.

### Intermediate 274

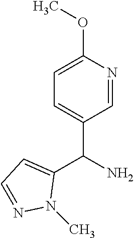 [SMILES: COc1ccc(C(N)c2ccnn2C)cn1]

The product is prepared according to General Procedure 14 described for Intermediate 12 with (6-methoxy-pyridin-3-yl)-(2-methyl-2H-pyrazol-3-yl)-methanone (Intermediate 273) (420.00 mg; 1.93 mmol; 1.00 eq.), TTIP (1.14 mL; 3.87 mmol; 2.00 eq.), NaBH₄ (292.60 mg; 7.73 mmol; 4.00 eq.) and 7M NH₃ in MeOH (4 mL). Crude C-(6-methoxy-pyridin-3-yl)-C-(2-methyl-2H-pyrazol-3-yl)-methylamine (320.00 mg; yield 51.9%; beige solid) is used in the next step.

### Example 301

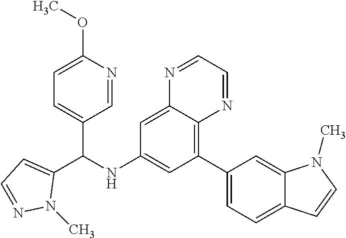 [SMILES: COc1ccc(C(Nc2cc(-c3ccc4ccn(C)c4c3)c3nccnc3c2)c2ccnn2C)cn1]

The product is prepared according to General Procedure 2 described in Example 1 with 7-chloro-5-(1-methyl-1H-indol-6-yl)-quinoxaline (Intermediate 4) (60.00 mg; 0.20 mmol; 1.00 eq.), C-(6-methoxy-pyridin-3-yl)-C-(2-methyl-2H-pyrazol-3-yl)-methylamine (Intermediate 274) (89.16 mg; 0.41 mmol; 2.00 eq.), Pd₂(dba)₃ (18.70 mg; 0.02 mmol; 0.10 eq.), BINAP (25.44 mg; 0.04 mmol; 0.20 eq.), NaOtBu (78.43 mg; 0.82 mmol; 4.00 eq.) in toluene (5 mL). Purification by FCC (DCM/MeOH; gradient). [(6-Methoxy-pyridin-3-yl)-(2-methyl-2H-pyrazol-3-yl)-methyl]-[8-(1-methyl-1H-indol-6-yl)-quinoxalin-6-yl]-amine (75.00 mg; yield 77.2%; 97% by HPLC) is obtained as a brown powder.

### Example 302

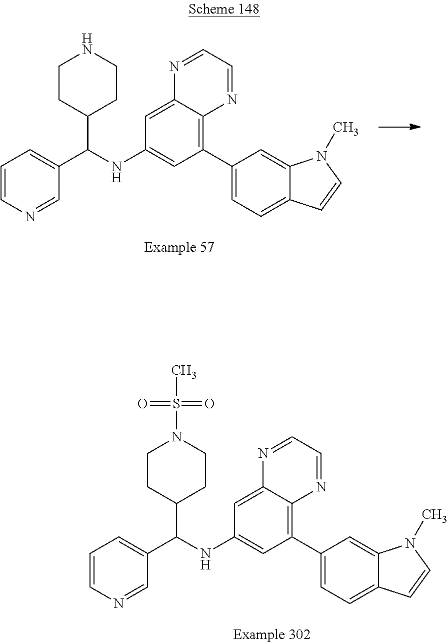 [SMILES: Cn1ccc2ccc(-c3cc(NC(c4cccnc4)C4CCNCC4)cc4nccnc34)cc21 ; Cn1ccc2ccc(-c3cc(NC(c4cccnc4)C4CCN(S(C)(=O)=O)CC4)cc4nccnc34)cc21]

The product is prepared according to General Procedure 35 described in Example 82 with [8-(1-methyl-1H-indol-6-yl)-quinoxalin-6-yl]-(piperidin-4-yl-pyridin-3-yl-methyl)-amine (Example 57) (70.00 mg; 0.16 mmol; 1.00 eq.), methanesulfonyl chloride (0.01 mL; 0.14 mmol; 0.90 eq.), TEA (0.02 mL; 0.14 mmol; 0.90 eq.) and DCM (1 mL). Purification by FCC (Column NH₂; DCM/MeOH; gradient). Repurification by preparative HPLC. [(1-Methanesulfonyl-piperidin-4-yl)-pyridin-3-yl-methyl]-[8-(1-methyl-1H-indol-6-yl)-quinoxalin-6-yl]-amine formic acid (8 mg; yield 7.3%; 81% by HPLC) is obtained as a yellow solid.

### Example 303

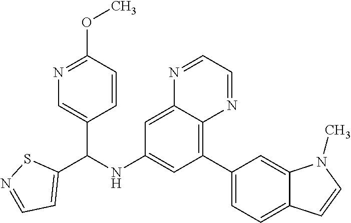 [SMILES: COc1ccc(C(Nc2cc(-c3ccc4ccn(C)c4c3)c3nccnc3c2)c2ccns2)cn1]

The product is prepared according to General Procedure 32 described in Example 1 with 7-chloro-5-(1-methyl-1H-indol-6-yl)-quinoxaline (Intermediate 4) (80.00 mg; 0.27 mmol; 1.00 eq.), C-isothiazol-5-yl-C-(6-methoxy-pyridin-3-yl)-methylamine (90.00 mg; 0.41 mmol; 1.49 eq.), NaOtBu (104.69 mg; 1.09 mmol; 4.00 eq.), bis(tri-tert-butylphosphine)palladium(0) (16.70 mg; 0.03 mmol; 0.12 eq.) and 1,4-dioxane (2 mL). Purification by FCC (DCM/MeOH; gradient). [Isothiazol-5-yl-(6-methoxy-pyridin-3-yl)-methyl]-[8-(1-methyl-1H-indol-6-yl)-quinoxalin-6-yl]-amine (34.00 mg; yield 24.1%; 92% by HPLC) is obtained as a yellow powder.

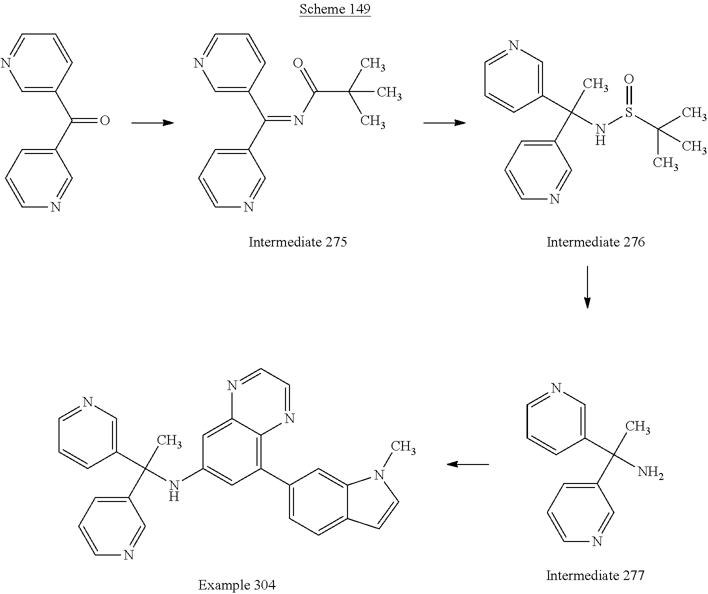 [SMILES: O=C(c1cccnc1)c1cccnc1 ; CC(C)(C)C(=O)N=C(c1cccnc1)c1cccnc1 ; CC(NS(=O)C(C)(C)C)(c1cccnc1)c1cccnc1 ; Cn1ccc2ccc(-c3cc(NC(C)(c4cccnc4)c4cccnc4)cc4nccnc34)cc21 ; CC(N)(c1cccnc1)c1cccnc1]

### Intermediate 275

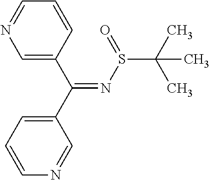 [SMILES: CC(C)(C)S(=O)N=C(c1cccnc1)c1cccnc1]

A sealed tube is charged with di-pyridin-3-yl-methanone (700.00 mg; 2.90 mmol; 1.00 eq.), 2-methyl-propane-2-sulfinic acid amide (527.17 mg; 4.35 mmol; 1.50 eq.) and THF anhydrous (20 mL). Then Ti(OEt)₄ (1.22 mL; 5.80 mmol; 2.00 eq.) is added via syringe and the reaction mixture is stirred at 85° C. for 48 h. The crude product is purified by FCC (DCM/MeOH; gradient). 2-Methyl-propane-2-sulfinic acid di-pyridin-3-yl-methyleneamide (460.00 mg; yield 51.6%) is obtained as a yellow oil.

### Intermediate 276

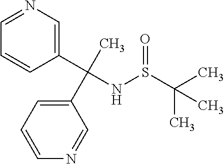 [SMILES: CC(NS(=O)C(C)(C)C)(c1cccnc1)c1cccnc1]

A CH₃BrMg (0.65 mL; 1.96 mmol; 3.00 eq.) is added dropwise at 0° C. to mixture of 2-methyl-propane-2-sulfinic acid di-pyridin-3-yl-methyleneamide (Intermediate 275) (200.00 mg; 0.65 mmol; 1.00 eq.) in dry THF (5 mL). RM is stirred at 0° C. for 1.5 h. The reaction is quenched with water at 0° C., extracted with DCM. Aqueous layer is extracted with iPrOH/DCM(1/4). The combined organic phases are washed with brine and then concentrated in vacuo. 2-Methyl-propane-2-sulfinic acid (1,1-di-pyridin-3-yl-ethyl)-amide (199.00 mg; 81.6%; yellow oil) is used in the next step without purification.

### Intermediate 277

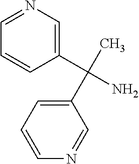

The product is prepared according to General Procedure 11 described in Example 46 with 2-methyl-propane-2-sulfinic acid (1,1-di-pyridin-3-yl-ethyl)-amide (Intermediate 276) (100.00 mg; 0.27 mmol; 1.00 eq.), 2M HCl in Et₂O (4.00 mL; 8.00 mmol; 29.82 eq.) and MeOH (4 mL). Crude 1,1-di-pyridin-3-yl-ethylamine hydrochloride (82.00 mg; yield 118.7%) is used in the next step.

### Example 304

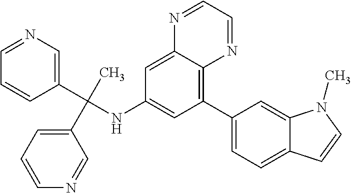 [SMILES: Cn1ccc2ccc(-c3cc(NC(C)(c4cccnc4)c4cccnc4)cc4nccnc34)cc21]

The product is prepared according to General Procedure 6 described for Intermediate 6 with 7-chloro-5-(1-methyl-1H-indol-6-yl)-quinoxaline (Intermediate 4) (36.00 mg; 0.12 mmol; 1.00 eq.), 1,1-di-pyridin-3-yl-ethylamine hydrochloride (Intermediate 277) (45.24 mg; 0.17 mmol; 1.50 eq.), Cs₂CO₃ (229.90 mg; 0.70 mmol; 6.00 eq.), BINAP (29.59 mg; 0.05 mmol; 0.40 eq.), Pd(OAc)₂ (11.01 mg; 0.05 mmol; 0.40 eq.) and 1,4-dioxane (5 mL). Purification by FCC (DCM/MeOH; gradient). Repurification by FCC (NH₂ column; hexane/EtOAc: gradient). (1,1-Di-pyridin-3-yl-ethyl)-[8-(1-methyl-1H-indol-6-yl)-quinoxalin-6-yl]-amine (5.00 mg; yield 9.2%; 97% by HPLC) is obtained as a yellow powder.

### Example 305

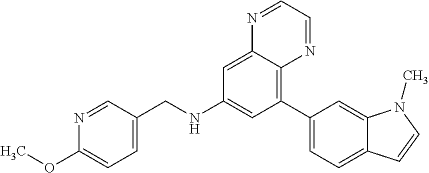 [SMILES: COc1ccc(CNc2cc(-c3ccc4ccn(C)c4c3)c3nccnc3c2)cn1]

The product is prepared according to General Procedure 22 described in Example 61 with 8-(1-methyl-1H-indol-6-yl)-quinoxalin-6-ylamine (Intermediate 22) (50.00 mg; 0.18 mmol; 1.00 eq.) and 6-methoxy-pyridine-3-carbaldehyde (27.99 μL; 0.24 mmol; 1.30 eq.), CH₃COOH (100.18 μL; 1.75 mmol; 9.60 eq.), NaBH(OAc)₃ (49.98 mg; 0.24 mmol; 1.30 eq.) and 1,2-dichloroethane (5 mL). Purification by FCC (DCM/MeOH; gradient). Repurification by preparative HPLC. (6-Methoxy-pyridin-3-ylmethyl)-[8-(1-methyl-1H-indol-6-yl)-quinoxalin-6-yl]-amine (15.00 mg; yield 20.6%; 99% by HPLC) is obtained as an orange powder.

### Example 306

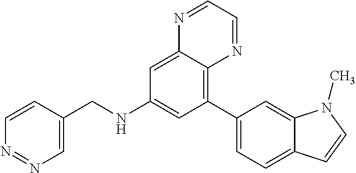 [SMILES: Cn1ccc2ccc(-c3cc(NCc4ccnnc4)cc4nccnc34)cc21]

The product is prepared according to General Procedure 23 described in Example 63 with 8-(1-methyl-1H-indol-6-yl)-quinoxalin-6-ylamine (Intermediate 22) (60.00 mg; 0.21 mmol; 1.00 eq.), pyridazine-4-carbaldehyde (23.17 mg; 0.21 mmol; 1.00 eq.), Hantzsch ester (67.87 mg; 0.27 mmol; 1.25 eq.), TMCS (5.44 μL; 0.04 mmol; 0.20 eq.) and DCM anhydrous (2 mL). Purification by FCC (column NH₂ 30 UM; hexane/EtOAc; gradient). [8-(1-Methyl-1H-indol-6-yl)-quinoxalin-6-yl]-pyridazin-4-ylmethyl-amine (30.00 mg; yield 33.2%; 87% by HPLC) is obtained as a brown powder.

### Example 307 & Example 308

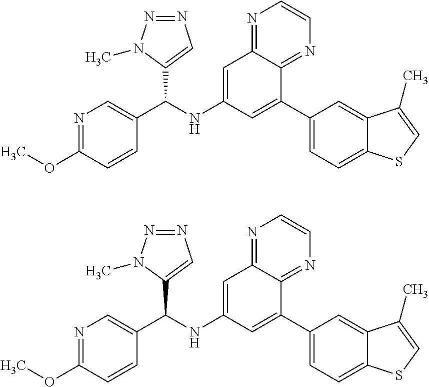 [SMILES: COc1ccc([C@@H](Nc2cc(-c3ccc4scc(C)c4c3)c3nccnc3c2)c2cnnn2C)cn1 ; COc1ccc([C@H](Nc2cc(-c3ccc4scc(C)c4c3)c3nccnc3c2)c2cnnn2C)cn1]

The preparative separation of the racemate: [(6-methoxy-pyridin-3-yl)-(3-methyl-3H-[1,2,3]triazol-4-yl)-methyl]-[8-(3-methyl-benzo[b]thiophen-5-yl)-quinoxalin-6-yl]-amine (Example 286) (30.5 mg) is performed by preparative SFC (Chiralpak AD-H; eluent: CO₂: iPrOH; 60:40). The combined fractions are evaporated to dryness. The oily residues are dissolved in ACN, diluted with water and lyophilized. N—[(R)-(6-methoxypyridin-3-yl)(1-methyl-1H-1,2,3-triazol-5-yl)methyl]-8-(3-methyl-1-benzothiophen-5-yl)quinoxalin-6-amine (Example 307) (11 mg; yield 36%; 99% by HPLC) and N—[(S)-(6-methoxypyridin-3-yl)(1-methyl-1H-1,2,3-triazol-5-yl)methyl]-8-(3-methyl-1-benzothiophen-5-yl)quinoxalin-6-amine (Example 308) (13.0 mg; yield 43%, 99.5% by HPLC) are obtained as yellow powders.

### Example 309 & Example 310

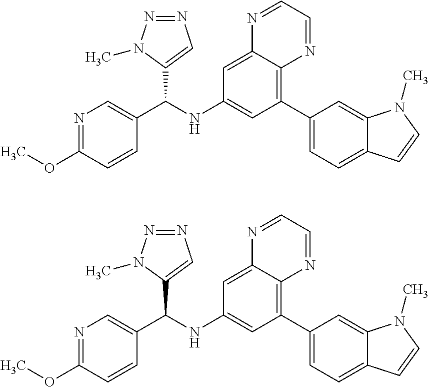 [SMILES: COc1ccc([C@@H](Nc2cc(-c3ccc4ccn(C)c4c3)c3nccnc3c2)c2cnnn2C)cn1 ; COc1ccc([C@H](Nc2cc(-c3ccc4ccn(C)c4c3)c3nccnc3c2)c2cnnn2C)cn1]

The preparative separation of the racemate [(6-methoxy-pyridin-3-yl)-(3-methyl-3H-[1,2,3]triazol-4-yl)-methyl]-[8-(1-methyl-1H-indol-6-yl)-quinoxalin-6-yl]-amine (44 mg) is performed by preparative SFC (column: ChiralPak AD-H; eluent: CO₂:iPrOH—60:40). The combined fractions are evaporated to dryness. The oily residues are dissolved in acetonitrile, diluted with water and lyophilized. N—[(R)-(6-methoxypyridin-3-yl)(1-methyl-1H-1,2,3-triazol-5-yl)methyl]-8-(1-methyl-1H-indol-6-yl)quinoxalin-6-amine (Example 309) (18 mg; yield 44%; 99.5% by HPLC) and N—[(S)-(6-methoxypyridin-3-yl)(1-methyl-1H-1,2,3-triazol-5-yl)methyl]-8-(1-methyl-1H-indol-6-yl)quinoxalin-6-amine (Example 310) (17 mg; yield 39%; 99% by HPLC) are obtained as yellow powders.

### Example 311 & Example 312

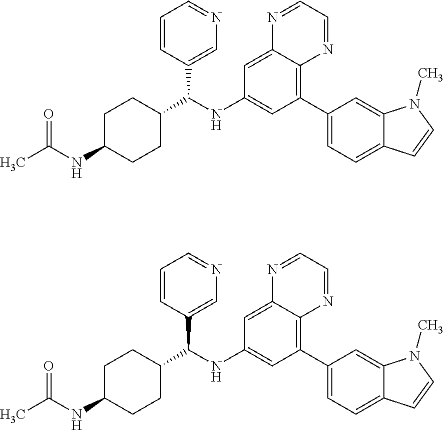 [SMILES: CC(=O)N[C@H]1CC[C@H]([C@@H](Nc2cc(-c3ccc4ccn(C)c4c3)c3nccnc3c2)c2cccnc2)CC1 ; CC(=O)N[C@H]1CC[C@H]([C@H](Nc2cc(-c3ccc4ccn(C)c4c3)c3nccnc3c2)c2cccnc2)CC1]

The preparative separation of the racemate N-(4-{[8-(1-methyl-1H-indol-6-yl)-quinoxalin-6-ylamino]-pyridin-3-yl-methyl}-cyclohexyl)-acetamide (Example 253) (69 mg) was performed by preparative SFC (column: ChiralPak AD-H; eluent: CO₂:EtOH—60:40). The combined fractions were evaporated to dryness. The oily residues were dissolved in acetonitrile, diluted with water and lyophilized. N-[(1R,4r)-4-[(R)-{[8-(1-methyl-1H-indol-6-yl)quinoxalin-6-yl]amino}(pyridin-3-yl)methyl]cyclohexyl]acetamide (Example 311) (24 mg; yield 35%; 100% by HPLC) and N-[(1S,4r)-4-[(S)-{[8-(1-methyl-1H-indol-6-yl)quinoxalin-6-yl]amino}(pyridin-3-yl)methyl]cyclohexyl]acetamide (Example 312) (24 mg; yield 35%; 99% by HPLC) are obtained as yellow powders.

TABLE 1
Analytical data of compounds according to the examples described
hereinabove.

| Cpd. | Ex. | | | | |
|---|---|---|---|---|---|
| No. | No. | MW | IUPAC name | LC-MS | ¹H-NMR |
| 1 | 52 | 404.51 | 8-(1-methyl-1H- | HPLC | 1H NMR (400 MHz, DMSO) δ |
| | | | indol-6-yl)-N-[(1R)- | 98.3%; | 8.63 (d, J = 1.9 Hz, 1H), 8.44 (d, |
| | | | 1,2,3,4- | m/z = 405.3 | J = 1.9 Hz, 1H), 7.64-7.57 (m, |
| | | | tetrahydronaphthalen- | [M + H]⁺ | 2H), 7.46 (d, J = 2.6 Hz, 1H), |
| | | | 1-yl]quinoxalin-6- | | 7.37 (t, 1 = 5.1 Hz, 2H), |
| | | | amine | | 7.27 (dd, J = 8.1, 1.4 Hz, 1H), |
| | | | | | 7.22-7.14 (m, 3H), 7.03-6.96 (m, |
| | | | | | 2H), 6.47 (d, J = 3.0 Hz, 1H), |
| | | | | | 4.87 (dd, J = 13.3, 5.6 Hz, 1H), |
| | | | | | 3.82 (s, 3H), 2.81 (dt, J = 12.3, |
| | | | | | 6.4 Hz, 2H), 2.08-1.81 (m, |
| | | | | | 4H). |
| 2 | 1 | 379.46 | 8-(1-methyl-1H- | HPLC | 1H NMR (400 MHz, DMSO) δ |
| | | | indol-6-yl)-N-[1- | 96.7%; | 8.73 (d, J = 2.0 Hz, 1H), 8.57 (d, |
| | | | (pyridin-3- | m/z = 380.2 | J = 1.9 Hz, 1H), 8.49-8.40 (m, |
| | | | | [M + H]⁺ | 2H), 7.87 (dt, J = 7.9, 1.8 Hz, |
| | | | | | 1H), 7.64-7.57 (m, 2H), |
| | | | | | 7.47 (d, J = 2.6 Hz, 1H), |
| | | | | | 7.42-7.34 (m, 2H), 7.31-7.23 (m, 2H), |
| | | | | | 6.63 (d, J = 2.5 Hz, 1H), 6.48 (d, |
| | | | | | J = 3.0 Hz, 1H), 4.83 (p, J = 6.8 Hz, |
| | | | | | 1H), 3.82 (s, 3H), 1.56 (d, J = 6.8 Hz, |
| | | | | | 3H). |
| 3 | 2 | 379.46 | 8-(1-methyl-1H- | HPLC | 1H NMR (400 MHz, DMSO) δ |
| | | | indol-6-yl)-N-[2- | 97.5%; | 8.65 (d, J = 1.9 Hz, 1H), 8.57 (d, |
| | | | (pyridin-3- | m/z = 380.2 | J = 1.9 Hz, 1H), 8.49-8.42 (m, |
| | | | yl)ethyl]quinoxalin-6- | [M + H]⁺ | 2H), 7.78 (dd, J = 5.8, 3.9 Hz, |
| | | | amine | | 1H), 7.66-7.57 (m, 2H), |
| | | | | | 7.42-7.33 (m, 3H), 7.26 (dd, J = 8.1, |
| | | | | | 1.4 Hz, 1H), 6.87 (d, J = 2.6 Hz, |
| | | | | | 1H), 6.78 (t, J = 5.5 Hz, 1H), |
| | | | | | 6.48 (d, J = 3.0 Hz, 1H), 3.82 (s, |
| | | | | | 3H), 3.52 (dd, J = 12.7, 7.0 Hz, |
| | | | | | 2H), 2.99 (t, J = 7.1 Hz, 2H). |
| 4 | 3 | 379.46 | 8-(1-methyl-1H- | HPLC | 1H NMR (400 MHz, DMSO) δ |
| | | | indol-6-yl)-N-[1- | 97.4%; | 8.57 (d, J = 18 Hz, 1H), 8.54 (d, |
| | | | (pyridin-4- | m/z = 380.2 | J = 5.9 Hz, 2H), 8.44 (d, J = 1.9 Hz, |
| | | | yl)ethyl]quinoxalin-6- | [M + H]⁺ | 1H), 7.65-7.56 (m, 2H), |
| | | | amine | | 7.48 (dd, J = 6.9, 4.3 Hz, 3H), |
| | | | | | 7.39 (d, J = 3.0 Hz, 1H), |
| | | | | | 7.33-7.23 (m, 2H), 6.55 (d, J = 2.4 Hz, |
| | | | | | 1H), 6.48 (d, J = 3.0 Hz, 1H), |
| | | | | | 4.82-4.71 (m, 1H), 3.82 (s, |
| | | | | | 3H), 1.53 (d, J = 6.8 Hz, 3H). |
| 5 | 4 | 379.46 | 8-(1-methyl-1H- | HPLC | 1H NMR (400 MHz, DMSO) δ |
| | | | indol-6-yl)-N-[1- | 99.9%; | 8.60-8.54 (m, 2H), 8.43 (d, J = 1.9 Hz, |
| | | | (pyridin-2- | m/z = 380.2 | 1H), 7.76 (td, J = 7.7, |
| | | | yl)ethyl]quinoxalin-6- | [M + H]⁺ | 1.8 Hz, 1H), 7.60 (dd, J = 4.3, |
| | | | amine | | 3.4 Hz, 2H), 7.48 (dd, J = 5.2, |
| | | | | | 2.6 Hz, 2H), 7.39 (d, J = 3.1 Hz, |
| | | | | | 1H), 7.30-7.23 (m, 3H), |
| | | | | | 6.59 (d, J = 2.5 Hz, 1H), 6.48 (dd, J = 3.1, |
| | | | | | 0.8 Hz, 1H), 4.74 (p, J = 6.7 Hz, |
| | | | | | 1H), 3.82 (s, 3H), 1.56 (d, J = 6.8 Hz, |
| | | | | | 3H). |
| 6 | 5 | 408.50 | N-[(1S)-1-(3- | HPLC | 1H NMR (400 MHz, DMSO) δ |
| | | | methoxyphenyl)ethyl]- | 98%; | 8.57 (d, J = 1.9 Hz, 1H), 8.43 (d, |
| | | | 8-(1-methyl-1H- | m/z = 409.2 | J = 1.9 Hz, 1H), 7.63-7.56 (m, |
| | | | indol-6-yl)quinoxalin- | [M + H]⁺ | 2H), 7.47 (d, J = 2.6 Hz, 1H), |
| | | | 6-amine | | 7.39 (d, J = 3.0 Hz, 1H), |
| | | | | | 7.28-7.24 (m, 2H), 7.08-7.02 (m, |
| | | | | | 2H), 6.83-6.76 (m, 1H), |
| | | | | | 6.61 (d, J = 2.5 Hz, 1H), 6.48 (dd, J = 3.0, |
| | | | | | 0.8 Hz, 1H), 4.68 (s, 1H), |
| | | | | | 3.82 (s, 3H), 3.74 (s, 3H), |
| | | | | | 3.18 (s, 1H), 1.52 (d, J = 6.8 Hz, 3H). |
| 7 | 35 | 406.48 | 2-methoxy-4-(7- | HPLC | 1H NMR (400 MHz, DMSO) δ |
| | | | {[(1R)-1,2,3,4- | 100%; | 8.68 (d, J = 1.9 Hz, 1H), 8.48 (d, |
| | | | tetrahydronaphthalen- | m/z = 407.3 | J = 1.9 Hz, 1H), 7.81 (d, J = 7.9 Hz, |
| | | | 1- | [M + H]+ | 1H), 7.48 (d, J = 2.5 Hz, 1H), |
| | | | yl]amino}quinoxalin- | | 7.42 (s, 1H), 7.32 (dt, J = 8.1, |
| | | | 5-yl)benzonitrile | | 3.9 Hz, 2H), 7.19 (dd, J = 6.2, |
| | | | | | 3.1 Hz, 2H), 7.08 (s, 1H), |
| | | | | | 7.07 (d, J = 2.5 Hz, 1H), 4.89 (s, 1H), |
| | | | | | 3.94 (s, 3H), 2.88-2.72 (m, |
| | | | | | 2H), 2.09-1.72 (m, 5H). |
| 8 | 88 | 405.49 | 8-(1-methyl-1H-1,3- | HPLC | not determined |
| | | | benzodiazol-6-yl)-N- | 97.1%; | |
| | | | [(1R)- | m/z = 406.3 | |
| | | | 1,2,3,4- | [M + H]+ | |
| | | | tetrahydronaphthalen- | | |
| | | | 1-yl]quinoxalin-6- | | |
| | | | amine | | |
| 9 | Intermediate | 309.79 | 8-chloro-N-[(1R)- | HPLC | 1H NMR (400 MHz, DMSO) δ |
| | 31 | | 1,2,3,4- | 100%; | 8.72 (d, J = 1.9 Hz, 1H), 8.56 (d, |
| | | | tetrahydronaphthalen- | m/z = 310.2 | J = 1.9 Hz, 1H), 7.59 (d, J = 2.5 Hz, |
| | | | 1- | [M + H]+ | 1H), 7.30 (d, J = 7.1 Hz, 1H), |
| | | | yl]quinoxalin-6- | | 7.25 z 7.10 (m, 3H), 6.98 (d, J = 2.4 Hz, |
| | | | amine | | 1H), 4.83 (s, 1H), 2.89 z |
| | | | | | 2.70 (m, 2H), 2.04 z 1.93 (m, |
| | | | | | 1H), 1.93 z 1.78 (m, 3H). |
| 10 | 6 | 365.43 | 8-(1-methyl-1H- | HPLC | 1H NMR (400 MHz, DMSO) δ |
| | | | indol-6-yl)-N- | 90.3%; | 8.69 (s, 1H), 8.61 (d, J = 1.9 Hz, |
| | | | (pyridin-3- | m/z = 366.2 | 1H), 8.55-8.34 (m, 3H), |
| | | | ylmethyl)quinoxalin- | [M + H]+ | 7.85 (d, J = 7.8 Hz, 1H), 7.60 (d, J = 9.5 Hz, |
| | | | 6-amine | | 2H), 7.47-7.40 (m, |
| | | | | | 1H), 7.38 (d, J = 3.1 Hz, 2H), |
| | | | | | 7.27 (d, J = 1.4 Hz, 1H), 6.80 (d, |
| | | | | | J = 2.6 Hz, 1H), 6.47 (d, J = 3.8 Hz, |
| | | | | | 1H), 4.53 (s, 2H), 3.81 (s, |
| | | | | | 3H). |
| 11 | 7 | 408.50 | N-[(1R)-1-(3- | HPLC | 1H NMR (400 MHz, DMSO) δ |
| | | | methoxyphenyl)ethyl]- | 95.9%; | 8.56 (d, J = 1.9 Hz, 1H), 8.42 (d, |
| | | | 8-(1-methyl-1H- | m/z = 409.3 | J = 1.9 Hz, 1H), 7.60 (dd, J = 6.8, |
| | | | indol-6-yl)quinoxalin- | [M + H]+ | 1.4 Hz, 2H), 7.45 (d, J = 2.6 Hz, |
| | | | 6-amine | | 1H), 7.39 (d, J = 3.0 Hz, 1H), |
| | | | | | 7.30-7.23 (m, 2H), 7.20 (d, J = 6.7 Hz, |
| | | | | | 1H), 7.05 (dd, J = 4.9, |
| | | | | | 3.3 Hz, 2H), 6.82-6.77 (m, |
| | | | | | 1H), 6.60 (d, J = 2.5 Hz, 1H), |
| | | | | | 6.47 (dd, J = 3.1, 0.8 Hz, 1H), |
| | | | | | 4.71-4.64 (m, 1H), 3.82 (s, |
| | | | | | 3H), 3.74 (s, 3H), 1.51 (d, J = 6.8 Hz, |
| | | | | | 3H). |
| 12 | 83 | 396.48 | 8-(4-amino-3- | HPLC | 1H NMR (400 MHz, DMSO) δ |
| | | | methoxyphenyl)-N- | 100%; | 8.60 (d, J = 1.9 Hz, 1H), 8.43 (d, |
| | | | [(1R)-1,2,3,4- | m/z = 397.2 | J = 1.9 Hz, 1H), 7.34 (dd, J = 10.5, |
| | | | tetrahydronaphthalen- | [M + H]+ | 4.8 Hz, 2H), |
| | | | 1-yl]quinoxalin-6- | | 7.23-7.12 (m, 3H), 7.07 (d, J = 1.8 Hz, |
| | | | amine | | 1H), 6.98 (dd, J = 8.0, 1.8 Hz, |
| | | | | | 1H), 6.95-6.87 (m, 2H), |
| | | | | | 6.70 (d, J = 8.0 Hz, 1H), |
| | | | | | 4.93-4.77 (m, 3H), 3.78 (s, 3H), |
| | | | | | 2.91-2.69 (m, 3H), 2.08-1.69 (m, |
| | | | | | 4H). |
| 13 | 84 | 381.47 | 8-(5-amino-6- | HPLC | 1H NMR (400 MHz, DMSO) δ |
| | | | methylpyridin-3-yl)- | 98.6%; | 8.64 (d, J = 1.9 Hz, 1H), 8.45 (d, |
| | | | N-[(1R)-1,2,3,4- | m/z = 382.3 | J = 1.9 Hz, 1H), 7.83 (s, 1H), |
| | | | tetrahydronaphthalen- | [M + H]+ | 7.37 (d, J = 2.6 Hz, 1H), 7.33 (s, |
| | | | 1-yl]quinoxalin-6- | | 1H), 7.20 z 7.18 (m, 1H), |
| | | | amine | | 7.17 (s, 1H), 7.15 (d, J = 1.9 Hz, 1H), |
| | | | | | 7.05 (d, J = 8.4 Hz, 1H), 7.00 (d, |
| | | | | | J = 2.5 Hz, 1H), 5.10 (s, 2H), |
| | | | | | 4.85 (dd, J = 13.2, 5.5 Hz, 1H), |
| | | | | | 2.82 (s, 2H), 2.33 (s, 3H), |
| | | | | | 1.92 (s, 4H). |
| 14 | 8 | 406.48 | N-(3,4-dihydro-2H-1- | HPLC | 1H NMR (400 MHz, DMSO) δ |
| | | | benzopyran-4-yl)-8- | 97.7%; | 8.65 (d, J = 1.6 Hz, 1H), 8.46 (d, |
| | | | (1-methyl- | m/z = 407.2 | J = 1.7 Hz, 1H), 7.59 (d, J = 10.3 Hz, |
| | | | 1H-indol-6- | [M + H]+ | 2H), 7.44 (d, J = 2.2 Hz, 1H), |
| | | | yl)quinoxalin-6- | | 7.37 (d, J = 2.9 Hz, 1H), 7.33 (d, |
| | | | amine | | J = 7.4 Hz, 1H), 7.25 (d, J = 8.1 Hz, |
| | | | | | 1H), 7.19 (t, J = 7.5 Hz, 1H), |
| | | | | | 7.13 (d, J = 8.0 Hz, 1H), 7.07 (s, |
| | | | | | 1H), 6.91 (t, J = 7.2 Hz, 1H), |
| | | | | | 6.84 (d, J = 8.2 Hz, 1H), 6.46 (d, |
| | | | | | J = 2.7 Hz, 1H), 4.94 (s, 1H), |
| | | | | | 4.37-4.15 (m, 2H), 3.80 (s, |
| | | | | | 3H), 2.22-2.04 (m, J = 22.1, |
| | | | | | 17.7 Hz, 2H). |
| 15 | 9 | 408.50 | N-[1-(4- | HPLC | 1H NMR (400 MHz, DMSO) δ |
| | | | methoxyphenyl)ethyl]- | 98.6%; | 8.55 (d, J = 1.9 Hz, 1H), 8.41 (d, |
| | | | 8-(1-methyl-1H- | m/z = 409.2 | J = 1.9 Hz, 1H), 7.58 (s, 1H), |
| | | | indol-6-yl)quinoxalin- | [M + H]+ | 7.44 (d, J = 2.6 Hz, 1H), |
| | | | 6-amine | | 7.39 (dd, J = 5.9, 2.8 Hz, 3H), |
| | | | | | 7.25 (dd, J = 8.0, 1.6 Hz, 1H), |
| | | | | | 7.16 (d, J = 6.6 Hz, 1H), 6.91 (d, J = 8.8 Hz, |
| | | | | | 2H), 6.59 (d, J = 2.5 Hz, |
| | | | | | 1H), 6.47 (dd, J = 3.1, 0.7 Hz, |
| | | | | | 1H), 4.65 (t, J = 6.7 Hz, 1H), |
| | | | | | 3.81 (s, 3H), 3.72 (s, 3H), |
| | | | | | 1.50 (d, J = 6.7 Hz, 3H). |
| 16 | 11 | 405.49 | 8-(1-methyl-1H- | HPLC | 1H NMR (400 MHz, DMSO) δ |
| | | | indol-6-yl)-N- | 99.4%; | 8.65 (d, J = 1.9 Hz, 1H), 8.52 (s, |
| | | | (5,6,7,8- | m/z = 406.3 | 1H), 8.47 (d, J = 1.9 Hz, 1H), |
| | | | tetrahydroisoquinolin- | [M + H]+ | 8.34 (d, J = 5.1 Hz, 1H), 7.63 (s, |
| | | | 8-yl)quinoxalin-6- | | 1H), 7.60 (d, J = 8.2 Hz, 1H), |
| | | | amine | | 7.45 (d, J = 2.6 Hz, 1H), 7.38 (d, |
| | | | | | J = 3.1 Hz, 1H), 7.27 (dd, J = 8.2, |
| | | | | | 1.4 Hz, 1H), 7.19 (d, J = 5.1 Hz, |
| | | | | | 1H), 7.06 (dd, J = 5.4, 2.8 Hz, |
| | | | | | 2H), 6.47 (dd, J = 3.1, 0.8 Hz, |
| | | | | | 1H), 4.98 (dd, J = 13.1, 5.2 Hz, |
| | | | | | 1H), 3.81 (s, 3H), |
| | | | | | 2.90-2.72 (m, 2H), 1.99 (t, J = 5.2 Hz, |
| | | | | | 2H), 1.94-1.81 (m, 2H). |
| 17 | 85 | 409.48 | 8-(2,3-dihydro-1,4- | HPLC | 1H NMR (400 MHz, DMSO) δ |
| | | | benzodioxin-6-yl)-N- | 94.8%; | 8.62 (d, J = 2.0 Hz, 1H), 8.45 (d, |
| | | | [(1R)- | m/z = 410.2 | J = 1.9 Hz, 1H), 7.37 (d, J = 2.6 Hz, |
| | | | 1,2,3,4- | [M + H]+ | 1H), 7.35-7.30 (m, 1H), |
| | | | tetrahydronaphthalen- | | 7.17 (ddd, J = 12.5, 7.1, 1.9 Hz, |
| | | | 1-yl]quinoxalin-6- | | 3H), 7.11 (d, J = 2.1 Hz, 1H), |
| | | | amine | | 7.04 (dd, J = 8.3, 2.1 Hz, 1H), |
| | | | | | 6.97 (dd, J = 5.3, 2.8 Hz, 2H), |
| | | | | | 6.93 (d, J = 8.3 Hz, 1H), |
| | | | | | 4.84 (dd, J = 13.1, 5.6 Hz, 1H), |
| | | | | | 4.29 (s, 4H), 2.87-2.72 (m, 2H), |
| | | | | | 2.04-1.77 (m, 4H). |
| 18 | 89 | 424.49 | 2-methoxy-4-(7- | HPLC | 1H NMR (400 MHz, DMSO) δ |
| | | | {[(1R)-1,2,3,4- | 92.4%; | 8.66 (d, J = 1.9 Hz, 1H), 8.46 (d, |
| | | | tetrahydronaphthalen- | m/z = 425.3 | J = 1.9 Hz, 1H), 7.87 (d, J = 8.0 Hz, |
| | | | 1- | [M + H]+ | 1H), 7.69 (s, 1H), 7.56 (s, |
| | | | yl]amino}quinoxalin- | | 1H), 7.46 (d, J = 2.6 Hz, 1H), |
| | | | 5-yl)benzamide | | 7.34 (d, J = 7.0 Hz, 1H), 7.32 (d, |
| | | | | | J = 1.4 Hz, 1H), 7.23 (dd, J = 7.9, |
| | | | | | 1.5 Hz, 1H), |
| | | | | | 7.20-7.18 (m, 1H), 7.17 (s, 1H), 7.11 (d, J = 40.3 Hz, |
| | | | | | 1H), 7.04 (d, J = 2.1 Hz, |
| | | | | | 1H), 4.88 (s, 1H), 3.91 (s, |
| | | | | | 3H), 2.87-2.75 (m, 2H), |
| | | | | | 2.05-1.79 (m, 5H). |
| 19 | 12 | 405.49 | 8-(1-methyl-1H- | HPLC | 1H NMR (400 MHz, DMSO) δ |
| | | | indol-6-yl)-N- | 94.6%; | 8.64 (d, J = 1.9 Hz, 1H), 8.46 (d, |
| | | | (5,6,7,8- | m/z = 406.2 | J = 1.9 Hz, 1H), 8.41 (dd, J = 4.7, |
| | | | tetrahydroquinolin- | [M + H]+ | 1.5 Hz, 1H), 7.72 (d, J = 6.6 Hz, |
| | | | 5-yl)quinoxalin-6- | | 1H), 7.61 (d, J = 0.5 Hz, 1H), |
| | | | amine | | 7.59 (d, J = 8.2 Hz, 1H), 7.43 (d, |
| | | | | | J = 2.6 Hz, 1H), 7.38 (d, J = 3.0 Hz, |
| | | | | | 1H), 7.26 (dd, J = 8.2, 1.4 Hz, |
| | | | | | 1H), 7.22 (dd, J = 7.8, 4.7 Hz, |
| | | | | | 1H), 7.04 (t, J = 6.2 Hz, 2H), |
| | | | | | 6.47 (dd, J = 3.1, 0.8 Hz, 1H), |
| | | | | | 4.96 (dd, J = 12.9, 5.5 Hz, 1H), |
| | | | | | 3.81 (s, 3H), 2.90 (d, J = 6.4 Hz, |
| | | | | | 2H), 2.07-1.88 (m, 4H). |
| 20 | 86 | 369.46 | 8-(1,3-dimethyl-1H- | HPLC | 1H NMR (400 MHz, DMSO) δ |
| | | | pyrazol-4-yl)-N-[(1R)- | 97.8%; | 8.62 (d, J = 1.9 Hz, 1H), 8.45 (d, |
| | | | 1,2,3,4- | m/z = 370.2 | J = 1.9 Hz, 1H), 7.97 (s, 1H), |
| | | | tetrahydronaphthalen- | [M + H]+ | 7.38 (d, J = 2.6 Hz, 1H), |
| | | | 1-yl]quinoxalin-6- | | 7.36-7.31 (m, 1H), 7.22-7.15 (m, |
| | | | amine | | 3H), 6.98 (d, J = 8.3 Hz, 1H), |
| | | | | | 6.91 (d, J = 2.5 Hz, 1H), 3.84 (s, |
| | | | | | 3H), 2.85-2.76 (m, 2H), |
| | | | | | 2.19 (s, 3H), 2.01-1.80 (m, 4H). |
| 21 | 14 | 399.49 | 2-{[8-(1-methyl-1H- | HPLC | 1H NMR (400 MHz, DMSO) δ |
| | | | indol-6-yl)quinoxalin- | 97.2%; | 8.64 (d, J = 1.9 Hz, 1H), 8.47 (d, |
| | | | 6- | m/z = 400.2 | J = 1.9 Hz, 1H), 7.63-7.58 (m, |
| | | | yl]amino}-1- | [M + H]+ | 2H), 7.47 (d, J = 2.6 Hz, 1H), |
| | | | (pyrrolidin-1- | | 7.39 (d, J = 3.1 Hz, 1H), |
| | | | yl)propan-1-one | | 7.26 (dd, J = 8.1, 1.5 Hz, 1H), |
| | | | | | 6.82 (d, J = 8.0 Hz, 1H), 6.75 (d, J = 2.6 Hz, |
| | | | | | 1H), 6.48 (dd, J = 3.0, |
| | | | | | 0.8 Hz, 1H), 4.51-4.43 (m, |
| | | | | | 1H), 3.82 (s, 3H), |
| | | | | | 3.78-3.53 (m, 2H), 3.40-3.33 (m, 2H), |
| | | | | | 1.98-1.91 (m, 2H), |
| | | | | | 1.85-1.77 (m, 2H), 1.39 (d, J = 6.7 Hz, |
| | | | | | 3H). |
| 22 | 15 | 386.49 | N-(2,2- | HPLC | ¹H NMR (400 MHz, DMSO) δ |
| | | | dimethyloxan-4-yl)- | 100%; | 8.62 (d, J = 1.9 Hz, 1H), 8.44 (d, |
| | | | 8-(1-methyl-1H- | m/z = 387.3 | J = 1.9 Hz, 1H), 7.64-7.57 (m, |
| | | | indol-6- | [M + H]+ | 2H), 7.37 (dd, J = 13.7, 2.8 Hz, |
| | | | yl)quinoxalin-6- | | 2H), 7.26 (dd, J = 8.1, 1.5 Hz, |
| | | | amine | | 1H), 6.89 (d, J = 2.5 Hz, 1H), |
| | | | | | 6.53-6.45 (m, 2H), 3.81 (s, |
| | | | | | 3H), 3.73 (ddd, J = 12.0, 11.0, |
| | | | | | 3.6 Hz, 2H), 2.05-1.93 (m, |
| | | | | | 2H), 1.37-1.22 (m, 5H), |
| | | | | | 1.19 (s, 3H). |
| 23 | 16 | 372.46 | 8-(1-methyl-1H- | HPLC | 1H NMR (400 MHz, DMSO) δ |
| | | | indol-6-yl)-N-(oxan- | 100%; | 8.63 (d, J = 1.9 Hz, 1H), 8.44 (d, |
| | | | 3- | m/z = 373.2 | J = 1.9 Hz, 1H), 7.64-7.61 (m, |
| | | | ylmethyl)quinoxalin- | [M + H]+ | 2H), 7.60 (dd, J = 8.2, 0.4 Hz, |
| | | | 6-amine | | 1H), 7.38 (dd, J = 4.3, 2.9 Hz, |
| | | | | | 2H), 7.26 (dd, J = 8.1, 1.5 Hz, |
| | | | | | 2H), 6.78 (d, J = 2.6 Hz, 1H), |
| | | | | | 6.71 (s, 1H), 6.47 (dd, J = 3.1, |
| | | | | | 0.8 Hz, 1H), 4.35 (t, J = 5.1 Hz, |
| | | | | | 1H), 3.96-3.90 (m, 1H), |
| | | | | | 3.82 (s, 3H), 3.75 (dt, J = 11.0, 3.6 Hz, |
| | | | | | 2H), 1.98-1.87 (m, 2H), |
| | | | | | 1.61 (s, 1H), 1.58-1.43 (m, |
| | | | | | 1H), 1.42-1.29 (m, 1H). |
| 24 | 87 | 396.48 | 8-(3-amino-4- | HPLC | 1H NMR (400 MHz, DMSO) δ: |
| | | | methoxyphenyl)-N- | 100%; | 8.65 (d, J = 1.9 Hz, 1H), 8.47 (d, |
| | | | [(1R)-1,2,3,4- | m/z = 397.3 | J = 1.9 Hz, 1H), 7.63 (s, 1H), |
| | | | tetrahydronaphthalen- | [M + H]+ | 7.60 (d, J = 8.2 Hz, 1H), 7.47 (s, |
| | | | 1-yl]quinoxalin-6- | | 1H), 7.44 (d, J = 2.6 Hz, 1H), |
| | | | amine | | 7.38 (d, J = 3.1 Hz, 1H), |
| | | | | | 7.28 (dd, J = 8.2, 1.4 Hz, 1H), |
| | | | | | 6.93 (d, J = 2.5 Hz, 1H), 6.73 (d, J = 7.6 Hz, |
| | | | | | 1H), 6.47 (dd, J = 3.1, |
| | | | | | 0.8 Hz, 1H), 5.76 (s, 1H), |
| | | | | | 4.00-3.93 (m, 1H), 3.82 (s, 3H), |
| | | | | | 3.52-3.45 (m, 1H), 3.15 (ddd, J = 12.0, |
| | | | | | 7.0, 1.8 Hz, 1H), 2.36 (t, J = 6.9 Hz, |
| | | | | | 2H), 2.16-2.06 (m, |
| | | | | | 1H), 1.85 (td, J = 15.5, 7.5 Hz, |
| | | | | | 1H). |
| 25 | 32 | 426.47 | 8-(4-methoxy-3- | HPLC | not determined |
| | | | nitrophenyl)-N-[(1R)- | 95.4%; | |
| | | | 1,2,3,4- | m/z = 427.2 | |
| | | | tetrahydronaphthalen- | [M + H]+ | |
| | | | 1-yl]quinoxalin-6- | | |
| | | | amine | | |
| 26 | 97 | 284.74 | 8-chloro-N-[1- | HPLC | 1H NMR (400 MHz, DMSO) δ |
| | | | (pyridin-3- | 96%; | 8.67 (d, J = 10.8 Hz, 2H), |
| | | | yl)ethyl]quinoxalin-6- | m/z = 285.2 | 8.54 (s, 1H), 8.43 (d, J = 6.3 Hz, 1H), |
| | | | amine | [M + H]+ | 7.81 (d, J = 7.9 Hz, 1H), 7.57 (s, |
| | | | | | 1H), 7.35 (d, J = 12.1 Hz, 2H), |
| | | | | | 6.60 (s, 1H), 4.81 (s, 1H), |
| | | | | | 1.53 (d, J = 6.8 Hz, 3H). |
| 27 | 17 | 371.46 | 8-(1-methyl-1H- | HPLC | 1H NMR (400 MHz, DMSO) δ |
| | | | indol-6-yl)-N-(1,3- | 91.5%; | 9.12 (d, J = 2.0 Hz, 1H), 8.63 (d, |
| | | | thiazol-4- | m/z = 372.2 | J = 2.0 Hz, 1H), 8.49 (d, J = 2.0 Hz, |
| | | | ylmethyl)quinoxalin- | [M + H]+ | 1H), 7.65-7.61 (m, 2H), |
| | | | 6-amine | | 7.60 (d, J = 0.6 Hz, 1H), 7.48 (d, |
| | | | | | J = 2.6 Hz, 1H), 7.39 (d, J = 3.1 Hz, |
| | | | | | 1H), 7.26 (dd, J = 8.1, 1.6 Hz, |
| | | | | | 1H), 6.87 (d, J = 2.6 Hz, 1H), |
| | | | | | 6.48 (dd, J = 3.1, 0.7 Hz, 1H), |
| | | | | | 4.63 (s, 2H), 3.82 (s, 3H). |
| 28 | 18 | 457.55 | 3-(1-{[8-(1-methyl- | HPLC | 1H NMR (400 MHz, DMSO) δ |
| | | | 1H-indol-6- | 98%; | 8.56 (d, J = 1.9 Hz, 1H), 8.43 (d, |
| | | | yl)quinoxalin-6- | m/z = 458.2 | J = 1.9 Hz, 1H), 7.96 (s, 1H), |
| | | | yl]amino}ethyl)benzene- | [M + H]+ | 7.72 (dd, J = 7.7, 1.6 Hz, 2H), |
| | | | 1-sulfonamide | | 7.63-7.53 (m, 3H), 7.48 (d, J = 2.5 Hz, |
| | | | | | 1H), 7.40-7.35 (m, |
| | | | | | 3H), 7.32 (d, J = 6.4 Hz, 1H), |
| | | | | | 7.25 (dd, J = 8.3, 1.3 Hz, 1H), |
| | | | | | 6.57 (d, J = 2.4 Hz, 1H), 6.47 (d, |
| | | | | | J = 2.4 Hz, 1H), 4.87-4.76 (m, |
| | | | | | 1H), 3.81 (s, 3H), 1.53 (d, J = 6.7 Hz, |
| | | | | | 3H). |
| 29 | 90 | 421.49 | 1-methyl-6-(7-{[(1R)- | HPLC | 1H NMR (400 MHz, DMSO) δ |
| | | | 1,2,3,4- | 91.3%; | 8.68 (d, J = 2.0 Hz, 1H), 8.40 (d, |
| | | | tetrahydronaphthalen- | m/z = 422.3 | J = 2.0 Hz, 1H), 7.41-7.33 (m, J = 6.5, |
| | | | 1- | [M + H]+ | 5.7 Hz, 4H), |
| | | | yl]amino}quinoxalin- | | 7.23-7.15 (m, J = 15.8, 6.2 Hz, 4H), |
| | | | 5-yl)-1H,6H,7H- | | 7.10 (d, J = 2.5 Hz, 1H), 6.52 (d, J = 5.1 Hz, |
| | | | pyrrolo[2,3- | | 1H), 6.34 (d, J = 2.8 Hz, |
| | | | c]pyridin-7-one | | 1H), 4.89 (d, J = 14.8 Hz, 1H), |
| | | | | | 4.03 (s, 3H), 2.88-2.72 (m, |
| | | | | | 2H), 1.93 (d, J = 48.7 Hz, 4H). |
| 30 | 19 | 354.40 | N-(furan-2- | HPLC | 1H NMR (400 MHz, DMSO) δ |
| | | | ylmethyl)-8-(1- | 97%; | 8.64 (d, J = 1.9 Hz, 1H), 8.47 (d, |
| | | | methyl-1H-indol-6- | m/z = 355.2 | J = 1.9 Hz, 1H), 7.63 (t, J = 1.3 Hz, |
| | | | yl)quinoxalin-6- | [M + H]+ | 1H), 7.62-7.57 (m, 2H), |
| | | | amine | | 7.42 (d, J = 2.6 Hz, 1H), 7.39 (d, |
| | | | | | J = 3.1 Hz, 1H), 7.26 (dd, J = 8.1, |
| | | | | | 1.5 Hz, 1H), 7.14 (t, J = 5.8 Hz, |
| | | | | | 1H), 6.94 (d, J = 2.6 Hz, 1H), |
| | | | | | 6.47 (dd, J = 3.0, 0.8 Hz, 1H), |
| | | | | | 6.43 (d, J = 1.4 Hz, 2H), 4.47 (d, |
| | | | | | J = 5.8 Hz, 2H), 3.81 (s, 3H). |
| 31 | 20 | 447.53 | 1-(4-{[8-(1-methyl- | HPLC | 1H NMR (400 MHz, DMSO) δ |
| | | | 1H-indol-6- | 96.5%; | 8.64 (d, J = 1.9 Hz, 1H), 8.48 (d, |
| | | | yl)quinoxalin-6- | m/z = 448.2 | J = 1.9 Hz, 1H), 7.68-7.58 (m, |
| | | | yl]amino}-1,2,3,4- | [M + H]+ | 3H), 7.49 (d, J = 2.6 Hz, 1H), |
| | | | tetrahydroquinolin- | | 7.39 (d, J = 3.1 Hz, 1H), 7.36 (d, |
| | | | 1-yl)ethan-1- | | J = 7.7 Hz, 1H), 7.30-7.27 (m, |
| | | | one | | 1H), 7.15 (dd, J = 11.9, 7.9 Hz, |
| | | | | | 2H), 6.97 (d, J = 2.4 Hz, 1H), |
| | | | | | 6.92 (d, J = 7.2 Hz, 1H), |
| | | | | | 6.52-6.44 (m, 2H), 3.78 (d, J = 28.6 Hz, |
| | | | | | 3H), 3.17 (d, J = 5.3 Hz, 2H), |
| | | | | | 2.32 (dd, J = 13.3, 5.5 Hz, 2H), |
| | | | | | 1.94 (s, 1H), 1.77 (dt, J = 11.3, |
| | | | | | 5.5 Hz, 1H). |
| 32 | 21 | 364.44 | N-benzyl-8-(1- | HPLC | 1H NMR (400 MHz, DMSO) δ |
| | | | methyl-1H-indol-6- | 96.3%; | 8.59 (d, J = 1.9 Hz, 1H), 8.44 (d, |
| | | | yl)quinoxalin-6- | m/z = 365.2 | J = 1.9 Hz, 1H), 7.61-7.58 (m, |
| | | | amine | [M + H]+ | 2H), 7.47-7.44 (m, 3H), |
| | | | | | 7.38-7.35 (m, 3H), 7.31-7.24 (m, |
| | | | | | 3H), 6.75 (d, J = 2.6 Hz, 1H), |
| | | | | | 6.47 (dd, J = 3.1, 0.7 Hz, 1H), |
| | | | | | 4.49 (d, J = 5.8 Hz, 2H), 3.81 (s, |
| | | | | | 3H). |
| 33 | 115 | 393.48 | 2-methyl-8-(1- | HPLC | 1H NMR (400 MHz, DMSO) δ |
| | | | methyl-1H-indol-6- | 95.2%; | 8.72 (d, J = 1.8 Hz, 1H), 8.50 (s, |
| | | | yl)-N-[1-(pyridin-3- | m/z = 394.4 | 1H), 8.44 (dd, J = 4.7, 1.7 Hz, |
| | | | yl)ethyl]quinoxalin-6- | [M + H]+ | 1H), 7.86 (dt, J = 7.9, 2.0 Hz, |
| | | | amine | | 1H), 7.65-7.62 (m, 1H), |
| | | | | | 7.60 (dd, J = 8.2, 0.5 Hz, 1H), |
| | | | | | 7.41 (d, J = 2.6 Hz, 1H), 7.39 (d, J = 3.1 Hz, |
| | | | | | 1H), 7.37-7.34 (m, |
| | | | | | 1H), 7.26 (dd, J = 8.2, 1.5 Hz, |
| | | | | | 1H), 7.08 (d, J = 6.9 Hz, 1H), |
| | | | | | 6.61 (d, J = 2.6 Hz, 1H), |
| | | | | | 6.47 (dd, J = 3.1, 0.8 Hz, 1H), |
| | | | | | 4.81-4.76 (m, 1H), 3.82 (s, 3H), |
| | | | | | 2.47 (s, 3H). |
| 34 | 114 | 393.48 | 3-methyl-8-(1- | HPLC | 1H NMR (400 MHz, DMSO) δ |
| | | | methyl-1H-indol-6- | 90.9%; | 8.73 (d, J = 1.8 Hz, 1H), |
| | | | yl)-N-[1-(pyridin-3- | m/z = 394.3 | 8.45 (dd, J = 4.8, 1.6 Hz, 1H), |
| | | | yl)ethyl]quinoxalin-6- | [M + H]+ | 8.34 (s, 1H), 7.87 (dd, J = 6.0, 1.9 Hz, |
| | | | amine | | 1H), 7.61-7.56 (m, 2H), |
| | | | | | 7.38 (d, J = 3.1 Hz, 1H), 7.36 (d, |
| | | | | | J = 2.6 Hz, 1H), 7.25 (dd, J = 8.3, |
| | | | | | 1.3 Hz, 1H), 7.18 (d, J = 6.8 Hz, |
| | | | | | 1H), 6.54 (d, J = 2.5 Hz, 1H), |
| | | | | | 6.47 (dd, J = 3.1, 0.7 Hz, 1H), |
| | | | | | 5.76 (s, 1H), 4.83-4.76 (m, |
| | | | | | 1H), 3.81 (s, 3H), 2.52 (s, 3H), |
| | | | | | 1.55 (d, J = 6.8 Hz, 3H), 1.24 (s, |
| | | | | | 1H). |
| 35 | 22 | 379.46 | 8-(1-methyl-1H- | HPLC | 1H NMR (400 MHz, DMSO) δ |
| | | | indol-6-yl)-N-[(1R)-1- | 97.0%; | 8.73 (d, J = 1.5 Hz, 1H), 8.57 (d, |
| | | | (pyridin-3- | m/z = 380.2 | J = 1.9 Hz, 1H), 8.48-8.41 (m, |
| | | | yl)ethyl]quinoxalin-6- | [M + H]+ | 2H), 7.87 (dt, J = 7.9, 1.9 Hz, |
| | | | amine | | 1H), 7.60 (dd, J = 4.1, 3.6 Hz, |
| | | | | | 2H), 7.46 (d, J = 2.6 Hz, 1H), |
| | | | | | 7.41-7.34 (m, 2H), 7.26 (dd, J = 8.2, |
| | | | | | 1.4 Hz, 2H), 6.62 (d, J = 2.6 Hz, |
| | | | | | 1H), 6.48 (dd, J = 3.1, |
| | | | | | 0.8 Hz, 1H), 4.83 (t, J = 6.8 Hz, |
| | | | | | 1H), 3.82 (s, 3H), 1.56 (d, J = 6.8 Hz, |
| | | | | | 3H). |
| 36 | 23 | 379.46 | 8-(1-methyl-1H- | HPLC | 1H NMR (400 MHz, DMSO) δ |
| | | | indol-6-yl)-N-[(1S)-1- | 95.1%; | 8.73 (d, J = 1.9 Hz, 1H), 8.57 (d, |
| | | | (pyridin-3- | m/z = 380.2 | J = 1.9 Hz, 1H), 8.46-8.44 (m, |
| | | | yl)ethyl]quinoxalin-6- | [M + H]+ | 1H), 8.44 (d, J = 1.9 Hz, 1H), |
| | | | amine | | 7.86 (dt, J = 7.9, 1.9 Hz, 1H), |
| | | | | | 7.60 (dd, J = 4.1, 3.6 Hz, 2H), |
| | | | | | 7.46 (d, J = 2.6 Hz, 1H), 7.39 (d, |
| | | | | | J = 3.0 Hz, 1H), 7.38-7.35 (m, |
| | | | | | 1H), 7.29-7.23 (m, 2H), |
| | | | | | 6.62 (d, J = 2.5 Hz, 1H), 6.48 (dd, J = 3.1, |
| | | | | | 0.8 Hz, 1H), 4.83 (t, J = 6.8 Hz, |
| | | | | | 1H), 3.82 (s, 3H), 1.56 (d, J = 6.8 Hz, |
| | | | | | 3H). |
| 37 | 24 | 380.45 | 8-(1-methyl-1H- | HPLC | 1H NMR (400 MHz, DMSO) δ |
| | | | indol-6-yl)-N-[1- | 94.9%; | 8.80 (d, J = 1.5 Hz, 1H), |
| | | | (pyrazin-2- | m/z = 381.2 | 8.66 (dd, J = 2.5, 1.5 Hz, 1H), |
| | | | yl)ethyl]quinoxalin-6- | [M + H]+ | 8.59 (d, J = 1.9 Hz, 1H), 8.55 (d, J = 2.6 Hz, |
| | | | amine | | 1H), 8.45 (d, J = 1.9 Hz, |
| | | | | | 1H), 7.60 (d, J = 7.9 Hz, 2H), |
| | | | | | 7.49 (d, J = 2.6 Hz, 1H), 7.39 (d, |
| | | | | | J = 3.1 Hz, 1H), 7.31 (d, J = 7.0 Hz, |
| | | | | | 1H), 7.26 (dd, J = 8.2, 1.4 Hz, |
| | | | | | 1H), 6.65 (d, J = 2.5 Hz, 1H), |
| | | | | | 6.48 (dd, J = 3.1, 0.6 Hz, 1H), |
| | | | | | 4.89 (p, J = 6.8 Hz, 1H), 3.82 (s, |
| | | | | | 3H), 1.61 (d, J = 6.8 Hz, 3H). |
| 38 | 112 | 275.3 | 8-(1-methyl-1H- | HPLC | 1H NMR (400 MHz, DMSO) δ |
| | | | indol-6-yl)quinoxalin- | 96%, | 10.55 (s, 1H), 8.79 (d, J = 1.8 Hz, |
| | | | 6-ol | m/z = 276.0 | 1H), 8.68 (d, J = 1.8 Hz, 1H), |
| | | | | [M + H]+ | 7.68-7.66 (m, 1H), 7.61 (dd, J = 8.2, |
| | | | | | 0.5 Hz, 1H), 7.46 (d, J = 2.7 Hz, |
| | | | | | 1H), 7.39 (d, J = 3.1 Hz, |
| | | | | | 1H), 7.30 (dd, J = 8.2, 1.5 Hz, |
| | | | | | 1H), 7.27 (d, J = 2.7 Hz, 1H), |
| | | | | | 6.48 (dd, J = 3.1, 0.8 Hz, 1H), |
| | | | | | 3.83 (s, 3H). |
| 39 | 25 | 357.45 | 8-(1-methyl-1H- | HPLC | 1H NMR (400 MHz, DMSO) δ |
| | | | indol-6-yl)-N- | 95.5%; | 8.61 (d, J = 1.9 Hz, 1H), 8.41 (d, |
| | | | (piperidin-3- | m/z = 358.2 | J = 1.9 Hz, 1H), 7.63 (s, 1H), |
| | | | yl)quinoxalin-6- | [M + H]+ | 7.59 (d, J = 8.1 Hz, 1H), 7.38 (d, |
| | | | amine | | J = 3.0 Hz, 1H), 7.36 (d, J = 2.6 Hz, |
| | | | | | 1H), 7.27 (dd, J = 8.2, 1.4 Hz, |
| | | | | | 1H), 6.89 (d, J = 2.5 Hz, 1H), |
| | | | | | 6.54 (d, J = 7.2 Hz, 1H), 6.47 (d, |
| | | | | | J = 3.0 Hz, 1H), 4.33 (d, J = 11.5 Hz, |
| | | | | | 1H), 3.89 (d, J = 12.9 Hz, |
| | | | | | 1H), 3.82 (s, 3H), 3.17 (s, 1H), |
| | | | | | 2.66 (dd, J = 11.0, 9.1 Hz, 2H), |
| | | | | | 2.48-2.30 (m, 2H), 1.99 (s, |
| | | | | | 2H), 1.42 (d, J = 8.8 Hz, 3H). |
| 40 | Intermediate | 274.32 | 8-(1-methyl-1H- | HPLC | 1H NMR (400 MHz, DMSO) δ |
| | 22 | | indol-6-yl)quinoxalin- | 96.7%; | 8.61 (d, J = 1.9 Hz, 1H), 8.44 (d, |
| | | | 6-amine | m/z | J = 1.9 Hz, 1H), 7.64-7.62 (m, |
| | | | | 275.0 | 1H), 7.60 (dd, J = 8.2, 0.5 Hz, |
| | | | | [M + H]+ | 1H), 7.38 (d, J = 3.1 Hz, 1H), |
| | | | | | 7.33 (d, J = 2.5 Hz, 1H), |
| | | | | | 7.26 (dd, J = 8.1, 1.5 Hz, 1H), |
| | | | | | 6.94 (d, J = 2.5 Hz, 1H), 6.47 (dd, J = 3.1, |
| | | | | | 0.8 Hz, 1H), 6.09 (s, 2H), |
| | | | | | 3.82 (s, 3H). |
| 41 | 91 | 366.42 | 8-(1H-indazol-6-yl)- | HPLC | 1H NMR (400 MHz, DMSO) δ |
| | | | N-[1-(pyridin-3- | 98.1%; | 13.14 (s, 1H), 8.73 (s, 1H), |
| | | | yl)ethyl]quinoxalin-6- | m/z = 367.2 | 8.59 (d, J = 1.9 Hz, 1H), 8.45 (d, J = 1.9 Hz, |
| | | | amine | [M + H]⁺ | 1H), 8.13 (s, 1H), |
| | | | | | 7.84 (dd, J = 15.5, 8.2 Hz, 2H), |
| | | | | | 7.73-7.70 (m, 1H), 7.49 (d, J = 2.6 Hz, |
| | | | | | 1H), 7.40-7.25 (m, 4H), |
| | | | | | 6.65 (d, J = 2.5 Hz, 1H), |
| | | | | | 4.87-4.80 (m, 1H), 1.56 (d, J = 6.8 Hz, |
| | | | | | 3H). |
| 42 | 113 | 366.42 | 5-(1-methyl-1H- | HPLC | 1H NMR (400 MHz, DMSO) δ |
| | | | indol-6-yl)-7- | 93.4%, | 8.89 (d, J = 1.9 Hz, 1H), 8.80 (s, |
| | | | (pyridin-3- | m/z = 367.1 | 1H), 8.79 (s, 1H), 8.59 (dd, J = 4.8, |
| | | | ylmethoxy)quinoxaline | [M + H]⁺ | 1.6 Hz, 1H), 8.54-8.43 (m, |
| | | | | | 1H), 8.00 (dt, J = 7.8, 1.8 Hz, |
| | | | | | 1H), 7.74-7.71 (m, 1H), |
| | | | | | 7.65 (d, J = 2.8 Hz, 1H), |
| | | | | | 7.64-7.61 (m, 1H), 7.60 (d, J = 2.8 Hz, |
| | | | | | 1H), 7.48 (ddd, J = 7.8, 4.8, 0.8 Hz, |
| | | | | | 1H), 7.40 (d, J = 3.0 Hz, 1H), |
| | | | | | 7.37 (dd, J = 8.2, 1.5 Hz, 1H), |
| | | | | | 6.48 (dd, J = 3.1, 0.8 Hz, 1H), |
| | | | | | 5.45 (s, 2H), 3.83 (s, 3H). |
| 43 | 101 | 380.45 | 8-{1-methyl-1H- | HPLC | 1H NMR (400 MHz, DMSO) δ |
| | | | pyrrolo[3,2- | 97.2%; | 8.73 (d, J = 1.9 Hz, 1H), 8.60 (d, |
| | | | b]pyridin-6-yl}-N-[1- | m/z = 381.2 | J = 1.9 Hz, 1H), 8.53 (d, J = 1.9 Hz, |
| | | | (pyridin-3- | [M + H]+ | 1H), 8.45 (dd, J = 5.3, 1.8 Hz, |
| | | | yl)ethyl]quinoxalin-6- | | 2H), 8.06 (dd, J = 1.8, 0.8 Hz, |
| | | | amine | | 1H), 7.87 (dt, J = 7.9, 1.9 Hz, |
| | | | | | 1H), 7.70 (dd, J = 9.8, 2.9 Hz, |
| | | | | | 1H), 7.51 (d, J = 2.6 Hz, 1H), |
| | | | | | 7.37 (dd, J = 7.8, 4.7 Hz, 1H), |
| | | | | | 7.31 (d, J = 6.9 Hz, 1H), 6.67 (d, |
| | | | | | J = 2.5 Hz, 1H), 6.61 (dd, J = 3.2, |
| | | | | | 0.8 Hz, 1H), 4.85 (p, J = 6.8 Hz, |
| | | | | | 1H), 3.86 (s, 3H), 1.56 (d, J = 6.8 Hz, |
| | | | | | 3H). |
| 47 | 59 | 442.51 | N-[bis(pyridin-3- | HPLC | 1H NMR (400 MHz, DMSO) δ |
| | | | yl)methyl]-8-(1- | 99.6%; | 8.77 (d, J = 2.0 Hz, 2H), 8.62 (d, |
| | | | methyl-1H-indol-6- | m/z = 443.3 | J = 1.9 Hz, 1H), 8.51 (s, 2H), |
| | | | yl)quinoxalin-6- | [M + H]⁺ | 8.50 (d, J = 1.9 Hz, 1H), |
| | | | amine | | 7.90 (dt, J = 8.0, 1.9 Hz, 2H), |
| | | | | | 7.64-7.57 (m, 4H), 7.46-7.40 (m, |
| | | | | | 2H), 7.39 (d, J = 3.0 Hz, 1H), |
| | | | | | 7.28 (dd, J = 8.2, 1.5 Hz, 1H), |
| | | | | | 6.85 (d, J = 2.5 Hz, 1H), |
| | | | | | 6.48 (dd, J = 3.1, 0.8 Hz, 1H), |
| | | | | | 6.19 (d, J = 7.1 Hz, 1H), 3.81 (s, 3H). |
| 48 | Intermediate | 347.80 | N-[bis(pyridin-3- | HPLC | not determined |
| | 21 | | yl)methyl]-8- | 99.2%; | |
| | | | chloroquinoxalin-6- | m/z = 348.2 | |
| | | | amine | [M + H]⁺ | |
| 49 | 94 | 380.45 | 8-{1-methyl-1H- | HPLC | not determined |
| | | | pyrrolo[2,3- | 100%; | |
| | | | b]pyridin-6-yl}-N-[1- | m/z = 381.3 | |
| | | | (pyridin-3- | [M + H]+ | |
| | | | yl)ethyl]quinoxalin-6- | | |
| | | | amine | | |
| 50 | 44 | 453.36 | 2,2,2-trifluoro-N-[8- | HPLC | 1H NMR (400 MHz, DMSO) δ |
| | | | (1-methyl-1H-indol- | 88.7%; | 8.64 (dd, J = 4.2, 1.8 Hz, 2H), |
| | | | 6- | m/z = 454.3 | 8.44 (d, J = 1.9 Hz, 1H), 8.25 (d, |
| | | | yl)quinoxalin-6-yl]-N- | [M + H]+ | J = 8.2 Hz, 1H), 7.87 (s, 1H), |
| | | | (piperidin-4- | | 7.60 (dd, J = 8.2, 1.4 Hz, 1H), |
| | | | yl)acetamide | | 7.39 (d, J = 2.6 Hz, 1H), 6.88 (d, |
| | | | | | J = 2.5 Hz, 1H), 6.65 (d, J = 7.9 Hz, |
| | | | | | 1H), 3.99 (s, 3H), 3.57 (s, |
| | | | | | 1H), 3.07 (d, J = 12.4 Hz, 2H), |
| | | | | | 2.71 (dd, J = 23.0, 11.3 Hz, 3H), |
| | | | | | 2.02 (dd, J = 25.0, 13.8 Hz, 3H). |
| 51 | 95 | 423.51 | 8-[1-(2- | HPLC | 1H NMR (400 MHz, DMSO) δ |
| | | | methoxyethyl)-1H- | 95.3%; | 8.73 (d, J = 1.9 Hz, 1H), 8.57 (d, |
| | | | indol-6-yl]-N-[1- | m/z = 424.3 | J = 1.9 Hz, 1H), 8.49-8.40 (m, |
| | | | (pyridin-3- | [M + H]+ | 2H), 7.87 (dt, J = 7.9, 1.9 Hz, |
| | | | yl)ethyl]quinoxalin-6- | | 1H), 7.65 (s, 1H), 7.59 (d, J = 8.1 Hz, |
| | | | amine | | 1H), 7.45 (d, J = 2.6 Hz, |
| | | | | | 1H), 7.41 (d, J = 3.1 Hz, 1H), |
| | | | | | 7.37 (dd, J = 7.4, 4.8 Hz, 1H), |
| | | | | | 7.25 (dd, J = 8.1, 1.3 Hz, 2H), |
| | | | | | 6.63 (d, J = 2.5 Hz, 1H), |
| | | | | | 6.48 (dd, J = 3.1, 0.6 Hz, 1H), |
| | | | | | 4.87-4.76 (m, 1H), 4.36 (t, J = 5.3 Hz, |
| | | | | | 2H), 3.68 (t, J = 5.3 Hz, 2H), |
| | | | | | 3.24 (s, 3H), 1.56 (d, J = 6.8 Hz, |
| | | | | | 3H). |
| 52 | 26 | 442.53 | N-[(4- | HPLC | 1H NMR (400 MHz, DMSO) δ |
| | | | methanesulfonylphenyl)methyl]- | 93.7%; | 8.61 (d, J = 1.9 Hz, 1H), 8.47 (d, |
| | | | 8-(1- | m/z = 443.4 | J = 1.9 Hz, 1H), 7.96-7.91 (m, |
| | | | methyl-1H-indol-6- | [M + H]+ | 2H), 7.74-7.70 (m, 2H), |
| | | | yl)quinoxalin-6- | | 7.64-7.59 (m, 2H), 7.46 (d, J = 2.6 Hz, |
| | | | amine | | 1H), 7.39 (d, J = 3.1 Hz, 1H), |
| | | | | | 7.27 (dd, J = 8.2, 1.4 Hz, 1H), |
| | | | | | 6.72 (d, J = 2.6 Hz, 1H), |
| | | | | | 6.48 (dd, J = 3.1, 0.5 Hz, 1H), |
| | | | | | 4.64 (s, 2H), 3.82 (s, 3H), 3.20 (s, |
| | | | | | 3H). |
| 53 | 27 | 366.42 | 8-(1-methyl-1H- | HPLC | 1H NMR (400 MHz, DMSO) δ |
| | | | indol-6-yl)-N- | 97.9%; | 9.17 (dd, J = 4.7, 1.8 Hz, 1H), |
| | | | (pyridazin-3- | m/z = 367.2 | 8.62 (d, J = 1.9 Hz, 1H), 8.47 (d, |
| | | | ylmethyl)quinoxalin- | [M + H]+ | J = 1.9 Hz, 1H), 7.74 (dd, J = 8.5, |
| | | | 6-amine | | 1.8 Hz, 1H), 7.69 (dd, J = 8.5, |
| | | | | | 4.7 Hz, 1H), 7.64-7.57 (m, |
| | | | | | 2H), 7.48 (d, J = 2.6 Hz, 2H), |
| | | | | | 7.39 (d, J = 3.1 Hz, 1H), |
| | | | | | 7.27 (dd, J = 8.1, 1.5 Hz, 1H), |
| | | | | | 6.80 (d, J = 2.6 Hz, 1H), 6.48 (dd, J = 3.1, |
| | | | | | 0.8 Hz, 1H), 4.81 (d, J = 5.7 Hz, |
| | | | | | 2H), 3.82 (s, 3H). |
| 54 | 28 | 442.53 | N-[(3- | HPLC | 1H NMR (400 MHz, DMSO) δ |
| | | | methanesulfonylphenyl)methyl]- | 97.7%; | 9.17 (dd, J = 4.7, 1.8 Hz, 1H), |
| | | | 8-(1- | m/z = 443.4 | 8.62 (d, J = 1.9 Hz, 1H), 8.47 (d, |
| | | | methyl-1H- | [M + H]+ | J = 1.9 Hz, 1H), 7.74 (dd, J = 8.5, |
| | | | indol-6-yl)quinoxalin- | | 1.8 Hz, 1H), 7.69 (dd, J = 8.5, |
| | | | 6-amine | | 4.7 Hz, 1H), 7.64-7.57 (m, |
| | | | | | 2H), 7.48 (d, J = 2.6 Hz, 2H), |
| | | | | | 7.39 (d, J = 3.1 Hz, 1H), |
| | | | | | 7.27 (dd, J = 8.1, 1.5 Hz, 1H), |
| | | | | | 6.80 (d, J = 2.6 Hz, 1H), 6.48 (dd, J = 3.1, |
| | | | | | 0.8 Hz, 1H), 4.81 (d, J = 5.7 Hz, |
| | | | | | 2H), 3.82 (s, 3H). |
| 55 | 29 | 442.53 | N-[(2- | HPLC | 1H NMR (400 MHz, DMSO) δ |
| | | | methanesulfonylphenyl)methyl]- | 98.1%; | 8.62 (d, J = 1.9 Hz, 1H), 8.49 (d, |
| | | | 8-(1- | m/z = 443.3 | J = 1.9 Hz, 1H), 8.04-8.01 (m, |
| | | | methyl-1H- | [M + H]+ | 1H), 7.74-7.68 (m, 2H), |
| | | | indol-6-yl)quinoxalin- | | 7.65-7.55 (m, 4H), 7.51 (d, J = 2.6 Hz, |
| | | | 6-amine | | 1H), 7.39 (d, J = 3.0 Hz, 1H), |
| | | | | | 7.29 (dd, J = 8.2, 1.5 Hz, 1H), |
| | | | | | 6.75 (d, J = 2.6 Hz, 1H), |
| | | | | | 6.48 (dd, J = 3.1, 0.8 Hz, 1H), |
| | | | | | 4.98 (s, 2H), 3.82 (s, 3H), 3.38 (s, |
| | | | | | 3H). |
| 56 | 30 | 371.48 | 8-(1-methyl-1H- | HPLC | 1H NMR (400 MHz, DMSO) δ |
| | | | indol-6-yl)-N- | 95.3%; | 8.62 (d, J = 1.9 Hz, 1H), 8.44 (d, |
| | | | (piperidin-2- | m/z = 372.3 | J = 1.9 Hz, 1H), 7.62 (s, 1H), |
| | | | ylmethyl)quinoxalin- | [M + H]+ | 7.60 (d, J = 8.2 Hz, 1H), |
| | | | 6-amine | | 7.39 (dd, J = 3.8, 2.9 Hz, 2H), |
| | | | | | 7.27 (dd, J = 8.1, 1.5 Hz, 1H), |
| | | | | | 6.82 (d, J = 2.6 Hz, 1H), 6.68 (t, J = 5.6 Hz, |
| | | | | | 1H), 6.47 (dd, J = 3.1, |
| | | | | | 0.7 Hz, 1H), 3.82 (s, 3H), |
| | | | | | 3.20-3.12 (m, 2H), 2.99 (d, J = 12.3 Hz, |
| | | | | | 1H), 2.82-2.72 (m, 2H), |
| | | | | | 1.75 (d, J = 10.9 Hz, 2H), |
| | | | | | 1.52 (s, 2H). |
| 57 | 31 | 371.48 | 8-(1-methyl-1H- | HPLC | 1H NMR (400 MHz, DMSO) δ |
| | | | indol-6-yl)-N- | 95.2%; | 8.73 (d, J = 1.8 Hz, 1H), 8.58 (d, |
| | | | (piperidin-3- | m/z = 372.3 | J = 1.9 Hz, 1H), 7.70 (d, J = 2.8 Hz, |
| | | | ylmethyl)quinoxalin- | [M + H]+ | 1H), 7.67 (s, 1H), 7.61 (d, J = 8.2 Hz, |
| | | | 6-amine | | 1H), 7.39 (d, J = 3.1 Hz, |
| | | | | | 1H), 7.30 (dd, J = 8.2, 1.4 Hz, |
| | | | | | 1H), 7.27 (d, J = 2.8 Hz, 1H), |
| | | | | | 6.90 (s, 1H), 6.48 (dd, J = 3.0, |
| | | | | | 0.7 Hz, 1H), 4.09 (d, J = 11.7 Hz, |
| | | | | | 1H), 3.98 (t, J = 6.6 Hz, 1H), |
| | | | | | 3.83 (s, 3H), 2.92 (t, J = 10.9 Hz, |
| | | | | | 1H), 2.78-2.58 (m, 3H), |
| | | | | | 1.80 (m, 2H), 1.65-1.48 (m, |
| | | | | | 1H), 0.86 (dd, J = 6.9, 5.9 Hz, |
| | | | | | 2H). |
| 58 | 96 | 381.43 | 5-(7-{[1-(pyridin-3- | HPLC | 1H NMR (400 MHz, DMSO): δ |
| | | | yl)ethyl]amino}quinoxalin- | 99.4%; | 8.72 (d, J = 1.9 Hz, 1H), 8.61 (d, |
| | | | 5- | m/z = 382.2 | J = 1.9 Hz, 2H), 8.45 (dd, J = 4.4, |
| | | | yl)-2,3-dihydro-1H- | [M + H]+ | 1.7 Hz, 2H), 7.85 (dt, J = 7.9, |
| | | | isoindol-1-one | | 1.9 Hz, 1H), 7.76 (d, J = 7.4 Hz, |
| | | | | | 2H), 7.69-7.63 (m, 1H), |
| | | | | | 7.47 (d, J = 2.6 Hz, 1H), |
| | | | | | 7.37 (dd, J = 7.4, 4.8 Hz, 1H), |
| | | | | | 7.30 (d, J = 6.9 Hz, 1H), 6.67 (d, J = 2.5 Hz, |
| | | | | | 1H), 4.83 (p, J = 6.7 Hz, |
| | | | | | 1H), 4.45 (s, 2H), 1.55 (d, J = 6.8 Hz, |
| | | | | | 3H). |
| 59 | 32 | 373.45 | 8-(1-methyl-1H- | HPLC | 1H NMR (400 MHz, DMSO) δ |
| | | | indol-6-yl)-N- | 99.5%; | 8.62 (d, J = 1.9 Hz, 1H), 8.44 (d, |
| | | | (morpholin-2- | m/z = 374.5 | J = 1.9 Hz, 1H), 7.71 z 7.54 (m, |
| | | | ylmethyl)quinoxalin- | [M + H]+ | 2H), 7.41 (d, J = 2.6 Hz, 1H), |
| | | | 6-amine | | 7.38 (d, J = 3.1 Hz, 1H), |
| | | | | | 7.26 (dd, J = 8.1, 1.4 Hz, 1H), |
| | | | | | 6.80 (d, J = 2.6 Hz, 1H), 6.69 (t, J = 5.7 Hz, |
| | | | | | 1H), 6.47 (d, J = 3.8 Hz, |
| | | | | | 1H), 3.81 (s, 3H), 3.77 (d, J = 10.8 Hz, |
| | | | | | 1H), 3.69 z 3.58 (m, |
| | | | | | 1H), 3.51 z 3.41 (m, 1H), 3.29 z |
| | | | | | 3.15 (m, 2H), 2.94 (d, J = 2.1 Hz, |
| | | | | | 1H), 2.67 (dd, J = 8.6, 2.7 Hz, |
| | | | | | 2H), 2.48 z 2.42 (m, 1H), |
| | | | | | 1.23 (s, 1H). |
| 60 | 61 | 354.41 | 8-(1-methyl-1H- | HPLC | 1H NMR (400 MHz, DMSO) δ |
| | | | indol-6-yl)-N-(1H- | 99.5%; | 8.63 (d, J = 1.9 Hz, 1H), 8.45 (d, |
| | | | pyrazol-4- | m/z = 355.2 | J = 1.9 Hz, 2H), 7.61 (s, 2H), |
| | | | ylmethyl)quinoxalin- | [M + H]⁺ | 7.59 (s, 1H), 7.42 (d, J = 2.6 Hz, |
| | | | 6-amine | | 1H), 7.38 (d, J = 3.1 Hz, 1H), |
| | | | | | 7.26 (d, J = 1.5 Hz, 1H), 7.24 (d, |
| | | | | | J = 1.5 Hz, 1H), 6.88 (dd, J = 6.8, |
| | | | | | 4.0 Hz, 2H), 6.47 (dd, J = 3.1, |
| | | | | | 0.7 Hz, 1H), 4.31 (d, J = 5.2 Hz, |
| | | | | | 2H), 3.81 (s, 3H). |
| 61 | 97 | 383.47 | 8-(1,3-benzothiazol- | HPLC | 1H NMR (400 MHz, DMSO) δ |
| | | | 6-yl)-N-[1-(pyridin-3- | 90.7%; | 9.45 (s, 1H), 8.72 (s, 1H), |
| | | | yl)ethyl]quinoxalin-6- | m/z = 384.2 | 8.61 (d, J = 1.7 Hz, 1H), |
| | | | amine | [M + H]+ | 8.49-8.41 (m, 2H), 8.37 (s, 1H), 8.17 (d, J = 8.4 Hz, |
| | | | | | 1H), 7.86 (d, J = 8.0 Hz, |
| | | | | | 1H), 7.74 (dd, J = 8.4, 1.5 Hz, |
| | | | | | 1H), 7.50 (d, J = 2.4 Hz, 1H), |
| | | | | | 7.36 (dd, J = 7.7, 4.8 Hz, 1H), |
| | | | | | 7.29 (d, J = 6.8 Hz, 1H), 6.67 (d, |
| | | | | | J = 2.2 Hz, 1H), 4.90-4.79 (m, |
| | | | | | 1H), 1.55 (d, J = 6.7 Hz, 3H). |
| 62 | 23 | 421.45 | 3-{[8-(1-methyl-1H- | HPLC | 1H NMR (400 MHz, DMSO) δ |
| | | | indol-6-yl)quinoxalin- | 98.4%; | 14.26 (s, 1H), 10.90 (s, 1H), |
| | | | 6-yl]amino}-3- | m/z = 422.2 | 10.81 (s, 1H), 9.24 (d, J = 1.7 Hz, |
| | | | (pyridin-3-yl)prop-2- | [M + H]+ | 1H), 9.02 (s, 1H), 8.93 (d, J = 1.8 Hz, |
| | | | enoic acid | | 1H), 8.90 (d, J = 1.8 Hz, |
| | | | | | 1H), 8.86 (dd, J = 4.8, 1.5 Hz, |
| | | | | | 1H), 8.84 (d, J = 1.8 Hz, 1H), |
| | | | | | 8.82 (d, J = 1.8 Hz, 1H), 8.77 (d, |
| | | | | | J = 3.6 Hz, 1H), 8.54 (d, J = 2.3 Hz, |
| | | | | | 1H), 8.50 (d, J = 2.3 Hz, 1H), |
| | | | | | 8.44-8.39 (m, 1H), |
| | | | | | 8.25-8.19 (m, 1H), 8.03 (d, J = 2.4 Hz, |
| | | | | | 1H), 7.95 (d, J = 2.4 Hz, 1H), |
| | | | | | 7.73 (s, 1H), 7.71 (s, 1H), |
| | | | | | 7.67 (d, J = 4.5 Hz, 1H), 7.64 (dd, J = 7.8, |
| | | | | | 3.9 Hz, 1H), 7.43 (d, J = 3.1 Hz, |
| | | | | | 1H), 7.41 (d, J = 3.0 Hz, 1H), |
| | | | | | 7.35 (dd, J = 8.2, 1.5 Hz, 1H), |
| | | | | | 7.32 (dd, J = 8.2, 1.5 Hz, 1H), |
| | | | | | 6.51 (dd, J = 3.1, 0.7 Hz, 1H), |
| | | | | | 6.50 (dd, J = 3.0, 0.7 Hz, 1H), |
| | | | | | 6.16 (s, 1H), 4.36 (s, 1H), |
| | | | | | 3.84 (s, 1H), 3.83 (s, J = 5.4 Hz, 1H). |
| 63 | 103 | 396.49 | 8-[3-(3- | HPLC | 1H NMR (400 MHz, DMSO) δ |
| | | | aminoazetidin-1- | 91.5%; | 8.70 (d, J = 2.0 Hz, 1H), 8.57 (d, |
| | | | yl)phenyl]-N-[1- | m/z = 397.5 | J = 1.9 Hz, 1H), 8.44 (dd, J = 4.7, |
| | | | (pyridin-3- | [M + H]+ | 1.6 Hz, 1H), 8.41 (d, J = 1.9 Hz, |
| | | | yl)ethyl]quinoxalin-6- | | 1H), 7.87-7.76 (m, 1H), |
| | | | amine | | 7.41-7.32 (m, 2H), 7.26 (dd, J = 14.7, |
| | | | | | 7.0 Hz, 2H), 6.90 (d, J = 8.8 Hz, |
| | | | | | 1H), 6.61 (dd, J = 4.9, |
| | | | | | 2.3 Hz, 2H), 6.53 (dd, J = 8.9, |
| | | | | | 2.4 Hz, 1H), 4.80 (t, J = 6.8 Hz, |
| | | | | | 1H), 4.09 (dd, J = 19.5, 12.0 Hz, |
| | | | | | 3H), 3.69 (dd, J = 7.8, 4.6 Hz, |
| | | | | | 2H), 1.91 (s, 1H), 1.55 (d, J = 6.8 Hz, |
| | | | | | 3H). |
| 64 | 102 | 409.48 | 1-[6-(7-{[1-(pyridin- | HPLC | 1H NMR (400 MHz, DMSO) δ |
| | | | 3- | 95.2%; | 8.71 (s, 1H), 8.56 (d, J = 1.8 Hz, |
| | | | yl)ethyl]amino}quinoxalin- | m/z = 410.3 | 1H), 8.44 (d, J = 3.9 Hz, 1H), |
| | | | 5- | [M + H]+ | 8.41 (d, J = 1.8 Hz, 1H), 8.25 (s, |
| | | | yl)-2,3-dihydro-1H- | | 1H), 7.84 (d, J = 7.9 Hz, 1H), |
| | | | indol-1-yl]ethan-1- | | 7.38 (d, J = 2.6 Hz, 1H), |
| | | | one | | 7.36-7.33 (m, 1H), 7.29 (t, J = 7.5 Hz, |
| | | | | | 2H), 7.17 (dd, J = 7.6, 1.4 Hz, |
| | | | | | 1H), 6.60 (d, J = 2.2 Hz, 1H), |
| | | | | | 4.85-4.74 (m, 1H), 4.16 (t, J = 8.5 Hz, |
| | | | | | 2H), 3.20 (t, J = 8.4 Hz, |
| | | | | | 2H), 2.17 (s, 3H), 1.54 (d, J = 6.7 Hz, |
| | | | | | 3H) |
| 65 | 98 | 359.47 | 8- | HPLC | 1H NMR (400 MHz, DMSO) δ |
| | | | {octahydrocyclopenta[c]pyrrol- | 93.3%; | 8.66 (s, 1H), 8.43 (d, J = 1.8 Hz, |
| | | | 2-yl}-N-[1- | m/z = 360.4 | 1H), 8.28 (d, J = 1.7 Hz, 1H), |
| | | | (pyridin-3- | [M + H]+ | 7.79 (d, J = 7.9 Hz, 1H), |
| | | | yl)ethyl]quinoxalin-6- | | 7.33 (dd, J = 7.7, 4.8 Hz, 1H), |
| | | | amine | | 6.92 (d, J = 6.9 Hz, 1H), 6.44 (d, J = 1.7 Hz, |
| | | | | | 1H), 6.10 (d, J = 1.7 Hz, |
| | | | | | 1H), 4.77-4.65 (m, 1H), |
| | | | | | 3.75 (dd, J = 9.5, 7.6 Hz, 1H), |
| | | | | | 3.68 (dd, J = 9.6, 7.6 Hz, 1H), |
| | | | | | 3.27 (dd, J = 12.7, 5.0 Hz, 2H), |
| | | | | | 2.73 (d, J = 2.6 Hz, 2H), 2.50 (s, 3H), |
| | | | | | 1.83-1.74 (m, 2H), 1.70 (dd, J = 12.7, |
| | | | | | 5.9 Hz, 1H), 1.56 (dd, J = 11.7, |
| | | | | | 6.1 Hz, 2H), 1.50 (d, J = 6.7 Hz, |
| | | | | | 2H |
| 66 | 33 | 358.44 | 8-(1-methyl-1H- | HPLC | 1H NMR (400 MHz, DMSO) δ |
| | | | indol-6-yl)-N-(oxan- | 96.4%; | 8.62 (d, J = 1.9 Hz, 1H), 8.44 (d, |
| | | | 4-yl)quinoxalin-6- | m/z = 359.2 | J = 1.9 Hz, 1H), 7.64-7.57 (m, |
| | | | amine | [M + H]+ | 2H), 7.38-7.35 (m, 2H), |
| | | | | | 7.26 (dd, J = 8.1, 1.5 Hz, 1H), |
| | | | | | 6.88 (d, J = 2.6 Hz, 1H), 6.62 (d, J = 7.8 Hz, |
| | | | | | 1H), 6.47 (dd, J = 3.1, |
| | | | | | 0.8 Hz, 1H), 3.91 (dt, J = 11.3, |
| | | | | | 3.4 Hz, 2H), 3.81 (s, 3H), |
| | | | | | 3.73-3.67 (m, 1H), 3.51 (td, J = 11.3, |
| | | | | | 2.0 Hz, 2H), 2.00 (dd, J = 12.8, |
| | | | | | 2.0 Hz, 2H), 1.57-1.41 (m, |
| | | | | | 2H). |
| 67 | 60 | 423.47 | 3-{[8-(1-methyl-1H- | HPLC | 1H NMR (400 MHz, DMSO) δ |
| | | | indol-6-yl)quinoxalin- | 99.5%; | 10.53 (s, 1H), 8.89 (d, J = 1.8 Hz, |
| | | | 6-yl]amino}-3- | m/z = 424.4 | 1H), 8.82 (s, 1H), 8.81 (d, J = 1.8 Hz, |
| | | | (pyridin-3- | [M + H]+ | 1H), 8.69 (d, J = 4.0 Hz, |
| | | | yl)propanoic acid | | 1H), 8.53 (d, J = 2.3 Hz, 1H), |
| | | | | | 8.29 (d, J = 7.5 Hz, 1H), 7.96 (d, |
| | | | | | J = 2.4 Hz, 1H), 7.82-7.75 (m, |
| | | | | | 1H), 7.69 (s, 1H), 7.65 (d, J = 8.2 Hz, |
| | | | | | 1H), 7.42 (d, J = 3.1 Hz, |
| | | | | | 1H), 7.31 (dd, J = 8.2, 1.5 Hz, |
| | | | | | 1H), 6.50 (dd, J = 3.1, 0.8 Hz, |
| | | | | | 1H), 5.31 (t, J = 6.7 Hz, 2H), |
| | | | | | 3.83 (s, 3H), 2.92-2.84 (m, |
| | | | | | 2H). |
| 68 | 99 | 394.43 | 6-(7-{[1-(pyridin-3- | HPLC | 1H NMR (400 MHz, DMSO) δ |
| | | | yl)ethyl]amino}quinoxalin- | 93.9%; | 8.73 (d, J = 1.9 Hz, 1H), 8.62 (d, |
| | | | 5- | m/z = 395.3 | J = 1.9 Hz, 1H), 8.47 (d, J = 1.9 Hz, |
| | | | yl)-4H-chromen-4- | [M + H]+ | 1H), 8.45 (dd, J = 4.7, 1.5 Hz, |
| | | | one | | 1H), 8.37 (d, J = 6.0 Hz, 1H), |
| | | | | | 8.23 (d, J = 2.1 Hz, 1H), |
| | | | | | 8.08 (dd, J = 8.7, 2.3 Hz, 1H), |
| | | | | | 7.86 (dt, J = 7.9, 1.9 Hz, 1H), 7.77 (d, |
| | | | | | J = 8.6 Hz, 1H), 7.55 (d, J = 2.6 Hz, |
| | | | | | 1H), 7.40-7.34 (m, 1H), |
| | | | | | 7.32 (d, J = 6.8 Hz, 1H), 6.67 (d, |
| | | | | | J = 2.5 Hz, 1H), 6.42 (d, J = 6.0 Hz, |
| | | | | | 1H), 4.84 (p, J = 6.8 Hz, 1H), |
| | | | | | 1.56 (d, J = 6.8 Hz, 3H). |
| 69 | 69 | 445.52 | 8-(1-methyl-1H- | HPLC | 1H NMR (400 MHz, DMSO) δ |
| | | | indol-6-yl)-N-{[5-(1- | 95.6%; | 8.74 (d, J = 2.1 Hz, 1H), 8.62 (d, |
| | | | methyl-1H- | m/z = 446.3 | J = 1.9 Hz, 1H), 8.50 (d, J = 1.9 Hz, |
| | | | pyrazol-4-yl)pyridin- | [M + H]+ | 1H), 8.47 (d, J = 1.9 Hz, 1H), |
| | | | 3- | | 8.25 (s, 1H), 8.04 (d, J = 1.9 Hz, |
| | | | yl]methyl}quinoxalin- | | 1H), 7.95 (d, J = 0.4 Hz, 1H), |
| | | | 6- | | 7.62 (s, 1H), 7.60 (d, J = 8.3 Hz, |
| | | | amine | | 1H), 7.46 (d, J = 2.6 Hz, 1H), |
| | | | | | 7.38 (d, J = 3.0 Hz, 1H), |
| | | | | | 7.31-7.23 (m, 2H), 6.85 (d, J = 2.5 Hz, |
| | | | | | 1H), 6.48 (dd, J = 3.0, 0.6 Hz, |
| | | | | | 1H), 4.53 (d, J = 5.6 Hz, 2H), |
| | | | | | 3.88 (s, 3H), 3.81 (s, 3H). |
| 70 | Intermediate 7 | 389.45 | 4-({[8-(1-methyl-1H- | HPLC | 1H NMR (400 MHz, DMSO) δ |
| | | | indol-6-yl)quinoxalin- | 98.6%; | 8.60 (d, J = 2.0 Hz, 1H), 8.46 (d, |
| | | | 6- | m/z = 390.3 | J = 2.0 Hz, 1H), 7.86-7.82 (m, |
| | | | yl]amino}methyl)benzonitrile | [M + H]⁺ | 2H), 7.64 (d, J = 8.6 Hz, 2H), |
| | | | | | 7.61 (s, 1H), 7.61-7.58 (m, |
| | | | | | 1H), 7.44 (d, J = 2.6 Hz, 1H), |
| | | | | | 7.41 (t, J = 6.5 Hz, 1H), 7.39 (d, |
| | | | | | J = 3.1 Hz, 1H), 7.26 (dd, J = 8.1, |
| | | | | | 1.5 Hz, 1H), 6.70 (d, J = 2.6 Hz, |
| | | | | | 1H), 6.47 (dd, J = 3.1, 0.7 Hz, |
| | | | | | 1H), 4.61 (d, J = 6.0 Hz, 2H), |
| | | | | | 3.81 (s, 3H). |
| 71 | Intermediate 6 | 389.45 | 3-({[8-(1-methyl-1H- | HPLC | 1H NMR (400 MHz, DMSO) δ |
| | | | indol-6-yl)quinoxalin- | 98.3%; | 8.61 (d, J = 1.9 Hz, 1H), 8.46 (d, |
| | | | 6- | m/z = 390.3 | J = 1.9 Hz, 1H), 7.91 (s, 1H), |
| | | | yl]amino}methyl)benzonitrile | [M + H]⁺ | 7.80 (d, J = 7.9 Hz, 1H), |
| | | | | | 7.75 (dt, J = 7.7, 1.3 Hz, 1H), |
| | | | | | 7.60 (ddd, J = 15.8, 6.7, 4.2 Hz, 3H), |
| | | | | | 7.44 (d, J = 2.6 Hz, 1H), 7.38 (d, |
| | | | | | J = 3.0 Hz, 1H), 7.35 (t, J = 6.0 Hz, |
| | | | | | 1H), 7.26 (dd, J = 8.1, 1.5 Hz, |
| | | | | | 1H), 6.75 (d, J = 2.6 Hz, 1H), |
| | | | | | 6.47 (dd, J = 3.1, 0.8 Hz, 1H), |
| | | | | | 4.57 (d, J = 6.0 Hz, 2H), 3.82 (s, |
| | | | | | 3H). |
| 72 | 70 | 431.49 | N-{[5-(1H-imidazol- | HPLC | 1H NMR (400 MHz, DMSO): δ |
| | | | 1-yl)pyridin-3- | 90.7%, | 8.88 (d, J = 2.5 Hz, 1H), 8.68 (d, |
| | | | yl]methyl}-8-(1- | m/z = 432.2 | J = 1.7 Hz, 1H), 8.63 (d, J = 1.9 Hz, |
| | | | methyl-1H-indol-6- | [M + H]⁺ | 1H), 8.48 (d, J = 1.9 Hz, 1H), |
| | | | yl)quinoxalin-6- | | 8.36 (s, 1H), 8.25-8.20 (m, |
| | | | amine | | 1H), 7.84 (t, J = 1.3 Hz, 1H), |
| | | | | | 7.63 (s, 1H), 7.60 (d, J = 8.2 Hz, |
| | | | | | 1H), 7.46 (d, J = 2.6 Hz, 1H), |
| | | | | | 7.39 (d, J = 3.1 Hz, 1H), 7.31 (t, |
| | | | | | J = 5.8 Hz, 1H), 7.27 (dd, J = 8.2, |
| | | | | | 1.5 Hz, 1H), 7.17 (s, 1H), |
| | | | | | 6.87 (d, J = 2.6 Hz, 1H), |
| | | | | | 6.48 (dd, J = 3.0, 0.8 Hz, 1H), |
| | | | | | 4.62 (d, J = 5.7 Hz, 2H), 3.81 (s, 3H). |
| 73 | 76 | 458.52 | N-{[5-(2- | HPLC | 1H NMR (400 MHz, DMSO): δ |
| | | | aminopyrimidin-5- | 91.9%, | 8.78 (d, J = 2.2 Hz, 1H), 8.65 (s, |
| | | | yl)pyridin-3- | m/z = 459.3 | 2H), 8.64-8.61 (m, 2H), |
| | | | yl]methyl}-8- | [M + H]⁺ | 8.47 (d, J = 1.9 Hz, 1H), 8.15 (t, J = 2.1 Hz, |
| | | | (1-methyl-1H-indol- | | 1H), 7.62 (s, 1H), |
| | | | 6-yl)quinoxalin-6- | | 7.60 (d, J = 8.2 Hz, 1H), 7.46 (d, J = 2.6 Hz, |
| | | | amine | | 1H), 7.38 (d, J = 3.1 Hz, |
| | | | | | 1H), 7.31-7.25 (m, 2H), |
| | | | | | 6.90 (d, J = 2.8 Hz, 2H), 6.87 (d, J = 2.6 Hz, |
| | | | | | 1H), 6.47 (dd, J = 3.0, |
| | | | | | 0.8 Hz, 1H), 4.57 (d, J = 5.7 Hz, |
| | | | | | 2H), 3.81 (s, 3H). |
| 74 | Intermediate | 409.44 | 8-(1-methyl-1H- | HPLC | 1H NMR (400 MHz, DMSO) δ |
| | 27 | | indol-6-yl)-N-[(4- | 92.9%, | 8.60 (d, J = 1.9 Hz, 1H), 8.46 (d, |
| | | | nitrophenyl)methyl]quinoxalin- | m/z = 410.3 | J = 1.9 Hz, 1H), 8.25 (d, J = 8.8 Hz, |
| | | | 6-amine | [M + H]⁺ | 2H), 7.72 (d, J = 8.8 Hz, 2H), |
| | | | | | 7.64-7.58 (m, 2H), 7.46 (d, J = 2.7 Hz, |
| | | | | | 2H), 7.39 (d, J = 3.1 Hz, |
| | | | | | 1H), 7.27 (dd, J = 8.1, 1.5 Hz, |
| | | | | | 1H), 6.71 (d, J = 2.6 Hz, 1H), |
| | | | | | 6.48 (dd, J = 3.1, 0.7 Hz, 1H), |
| | | | | | 4.67 (d, J = 6.0 Hz, 2H), 3.82 (s, |
| | | | | | 3H). |
| 75 | 80 | 379.46 | N-[(4- | HPLC | not determined |
| | | | aminophenyl)methyl]- | 84.4%, | |
| | | | 8-(1-methyl-1H- | m/z = 380.3 | |
| | | | indol-6- | [M + H]⁺ | |
| | | | yl)quinoxalin-6- | | |
| | | | amine | | |
| 76 | 39 | 409.48 | N-[1-(6- | HPLC | 1H NMR (400 MHz, DMSO) δ |
| | | | methoxypyridin-3- | 96.6%, | 8.58 (d, J = 1.9 Hz, 1H), 8.43 (d, |
| | | | yl)ethyl]-8-(1- | m/z = 410.3 | J = 1.9 Hz, 1H), 8.29 (d, J = 2.4 Hz, |
| | | | methyl-1H- | [M + H]⁺ | 1H), 7.78 (dd, J = 8.6, 2.5 Hz, |
| | | | indol-6-yl)quinoxalin- | | 1H), 7.65-7.56 (m, 2H), |
| | | | 6-amine | | 7.44 (d, J = 2.6 Hz, 1H), 7.39 (d, |
| | | | | | J = 3.0 Hz, 1H), 7.26 (dd, J = 8.2, |
| | | | | | 1.4 Hz, 1H), 7.17 (t, J = 9.8 Hz, |
| | | | | | 1H), 6.88-6.75 (m, 1H), |
| | | | | | 6.65 (d, J = 2.5 Hz, 1H), |
| | | | | | 6.47 (dd, J = 3.1, 0.8 Hz, 1H), |
| | | | | | 4.75 (p, J = 6.8 Hz, 1H), 3.82 (d, J = 0.5 Hz, |
| | | | | | 6H), 3.17 (d, J = 5.2 Hz, |
| | | | | | 1H), 1.53 (d, J = 6.8 Hz, 3H). |
| 77 | Intermediate | 409.44 | 8-(1-methyl-1H- | HPLC | 1H NMR (400 MHz, DMSO) δ |
| | 28 | | indol-6-yl)-N-[(3- | 93.4%, | 8.61 (d, J = 1.9 Hz, 1H), 8.47 (d, |
| | | | nitrophenyl)methyl]quinoxalin- | m/z = 410.3 | J = 1.9 Hz, 1H), 8.34 (s, 1H), |
| | | | 6-amine | [M + H]⁺ | 8.14 (d, J = 8.3 Hz, 1H), 7.94 (d, |
| | | | | | J = 7.5 Hz, 1H), 7.73-7.57 (m, |
| | | | | | 3H), 7.45 (dd, J = 9.7, 4.3 Hz, |
| | | | | | 2H), 7.39 (d, J = 3.1 Hz, 1H), |
| | | | | | 7.27 (dd, J = 8.1, 1.4 Hz, 1H), |
| | | | | | 6.77 (d, J = 2.6 Hz, 1H), 6.48 (d, |
| | | | | | J = 3.0 Hz, 1H), 4.67 (d, J = 5.7 Hz, |
| | | | | | 2H), 3.82 (s, 2H). |
| 78 | 81 | 379.46 | N-[(3- | HPLC | 1H NMR (400 MHz, DMSO) δ |
| | | | aminophenyl)methyl]- | 99.1%, | 8.59 (d, J = 1.9 Hz, 1H), 8.43 (d, |
| | | | 8-(1-methyl-1H- | m/z = 380.4 | J = 1.9 Hz, 1H), 7.60 (dd, J = 4.1, |
| | | | indol-6- | [M + H]⁺ | 3.5 Hz, 2H), 7.43 (d, J = 2.6 Hz, |
| | | | yl)quinoxalin-6- | | 1H), 7.38 (d, J = 3.1 Hz, 1H), |
| | | | amine | | 7.26 (dd, J = 8.2, 1.4 Hz, 1H), |
| | | | | | 7.21 (t, J = 5.8 Hz, 1H), 6.99 (t, |
| | | | | | J = 7.7 Hz, 1H), 6.74 (d, J = 2.6 Hz, |
| | | | | | 1H), 6.64 (d, J = 1.7 Hz, 1H), |
| | | | | | 6.59 (d, J = 7.6 Hz, 1H), |
| | | | | | 6.47 (dd, J = 3.1, 0.8 Hz, 1H), |
| | | | | | 6.45 (dd, J = 7.9, 1.3 Hz, 1H), |
| | | | | | 5.06 (s, 2H), 4.32 (d, J = 5.7 Hz, 2H), |
| | | | | | 3.82 (s, 3H). |
| 79 | 40 | 370.45 | 4-{[8-(1-methyl-1H- | HPLC | 1H NMR (400 MHz, DMSO) δ |
| | | | indol-6-yl)quinoxalin- | 100%, | 8.64 (d, J = 1.9 Hz, 1H), 8.46 (d, |
| | | | 6- | m/z = 371.3 | J = 1.9 Hz, 1H), 7.64-7.62 (m, |
| | | | yl]amino}cyclohexan- | [M + H]⁺ | 1H), 7.60 (dd, J = 8.2, 0.5 Hz, |
| | | | 1-one | | 1H), 7.41 (d, J = 2.6 Hz, 1H), |
| | | | | | 7.38 (d, J = 3.1 Hz, 1H), |
| | | | | | 7.27 (dd, J = 8.2, 1.5 Hz, 1H), |
| | | | | | 6.95 (d, J = 2.5 Hz, 1H), 6.71 (d, J = 7.6 Hz, |
| | | | | | 1H), 6.47 (dd, J = 3.1, |
| | | | | | 0.8 Hz, 1H), 4.06-3.96 (m, |
| | | | | | 1H), 3.82 (s, 3H), |
| | | | | | 2.62-2.52 (m, 2H), 2.38 (dt, J = 9.6, 4.3 Hz, |
| | | | | | 2H), 2.31-2.21 (m, 2H), |
| | | | | | 1.79 (td, J = 13.5, 4.8 Hz, 2H). |
| 80 | 41 | 371.44 | 5-{[8-(1-methyl-1H- | HPLC | 1H NMR (400 MHz, DMSO): δ |
| | | | indol-6-yl)quinoxalin- | 95.0%, | 8.65 (d, J = 1.9 Hz, 1H), 8.47 (d, |
| | | | 6- | m/z = 372.3 | J = 1.9 Hz, 1H), 7.63 (s, 1H), |
| | | | yl]amino}piperidin-2- | [M + H]⁺ | 7.60 (d, J = 8.2 Hz, 1H), 7.47 (s, |
| | | | one | | 1H), 7.44 (d, J = 2.6 Hz, 1H), |
| | | | | | 7.38 (d, J = 3.1 Hz, 1H), |
| | | | | | 7.28 (dd, J = 8.2, 1.4 Hz, 1H), |
| | | | | | 6.93 (d, J = 2.5 Hz, 1H), 6.73 (d, J = 7.6 Hz, |
| | | | | | 1H), 6.47 (dd, J = 3.1, |
| | | | | | 0.8 Hz, 1H), 5.76 (s, 1H), |
| | | | | | 4.00-3.93 (m, 1H), 3.82 (s, 3H), |
| | | | | | 3.52-3.45 (m, 1H), 3.15 (ddd, J = 12.0, |
| | | | | | 7.0, 1.8 Hz, 1H), 2.36 (t, J = 6.9 Hz, |
| | | | | | 2H), 2.16-2.06 (m, |
| | | | | | 1H), 1.85 (td, J = 15.5, 7.5 Hz, |
| | | | | | 1H). |
| 81 | 10 | 393.48 | 8-(1-methyl-1H-indol- | HPLC | 1H NMR (400 MHz, DMSO) δ |
| | | | 6-yl)-N-[2-(pyridin-3- | 100%, | 8.75 (d, J = 1.8 Hz, 1H), 8.51 (d, |
| | | | yl)propan-2-yl]quinoxalin- | m/z = 394.2 | J = 1.9 Hz, 1H), 8.49 (dd, J = 4.7, |
| | | | 6-amine | [M + H]⁺ | 1.6 Hz, 1H), 8.42 (d, J = 1.9 Hz, |
| | | | | | 1H), 7.93-7.87 (m, 1H), |
| | | | | | 7.59 (d, J = 8.2 Hz, 1H), 7.57 (s, |
| | | | | | 1H), 7.46 (d, J = 2.6 Hz, 1H), |
| | | | | | 7.42-7.39 (m, 1H), 7.38 (s, |
| | | | | | 1H), 7.25 (dd, J = 8.1, 1.5 Hz, |
| | | | | | 1H), 7.14 (s, 1H), 6.47 (dd, J = 3.1, |
| | | | | | 0.8 Hz, 1H), 6.17 (d, J = 2.6 Hz, |
| | | | | | 1H), 3.82 (s, 3H), 1.74 (s, |
| | | | | | 6H). |
| 82 | 100 | 379.46 | 8-(1-methyl-1H- | HPLC | 1H NMR (400 MHz, DMSO) δ |
| | | | indol-5-yl)-N-[1- | 99.9%, | 8.70 (d, J = 1.8 Hz, 1H), 8.57 (d, |
| | | | (pyridin-3- | m/z = 380.4 | J = 1.9 Hz, 1H), 8.54 (s, 1H), |
| | | | yl)ethyl]quinoxalin-6- | [M + H]⁺ | 8.46 (s, 1H), 8.31 (d, J = 5.6 Hz, |
| | | | amine | | 1H), 7.66 (s, 1H), |
| | | | | | 7.64-7.59 (m, 2H), 7.40 (d, J = 3.0 Hz, |
| | | | | | 1H), 7.29 (dd, J = 8.2, 1.5 Hz, |
| | | | | | 1H), 7.21 (d, J = 5.6 Hz, 1H), |
| | | | | | 7.05 (d, J = 2.6 Hz, 1H), |
| | | | | | 6.48 (dd, J = 3.1, 0.8 Hz, 1H), |
| | | | | | 3.92 (s, 3H), 3.82 (s, 3H). |
| 84 | 35 | 407.47 | 3-({[8-(1-methyl-1H- | HPLC | 1H NMR (400 MHz, DMSO) δ |
| | | | indol-6-yl)quinoxalin- | 96.3%, | 8.59 (d, J = 1.7 Hz, 1H), 8.44 (d, |
| | | | 6- | m/z = 408.3 | J = 1.7 Hz, 1H), 7.99 (s, 2H), |
| | | | yl]amino}methyl)benzamide | [M + H]⁺ | 7.77 (d, J = 7.8 Hz, 1H), |
| | | | | | 7.66-7.54 (m, J = 7.8 Hz, 3H), |
| | | | | | 7.44 (dd, J = 7.3, 2.4 Hz, 2H), |
| | | | | | 7.38 (d, J = 3.0 Hz, 1H), 7.36 (s, 1H), |
| | | | | | 7.33 (t, J = 5.9 Hz, 1H), |
| | | | | | 7.26 (dd, J = 8.1 Hz, 1H), 6.75 (d, J = 2.4 Hz, |
| | | | | | 1H), 6.47 (d, J = 3.0 Hz, |
| | | | | | 1H), 4.53 (d, J = 5.6 Hz, 2H), |
| | | | | | 3.81 (s, 3H). |
| 85 | 36 | 432.48 | 8-(1-methyl-1H- | HPLC | 1H NMR (400 MHz, DMSO) δ |
| | | | indol-6-yl)-N-{[3-(1H- | 92.7%, | 8.59 (d, J = 1.9 Hz, 1H), 8.45 (d, |
| | | | 1,2,3,4- | m/z = 433.3 | J = 1.9 Hz, 1H), 8.18 (s, 1H), |
| | | | tetrazol-5- | [M + H]⁺ | 7.94 (d, J = 7.6 Hz, 1H), 7.68 (d, |
| | | | yl)phenyl]methyl}quinoxalin- | | J = 7.8 Hz, 1H), 7.64-7.58 (m, J = 8.2, |
| | | | 6-amine | | 7.7 Hz, 3H), 7.47 (d, J = 2.6 Hz, |
| | | | | | 1H), 7.41 (t, J = 5.9 Hz, |
| | | | | | 1H), 7.38 (d, J = 3.0 Hz, 1H), |
| | | | | | 7.26 (dd, J = 8.1, 1.4 Hz, 1H), |
| | | | | | 6.78 (d, J = 2.5 Hz, 1H), 6.47 (d, |
| | | | | | J = 3.0 Hz, 1H), 4.61 (d, J = 5.7 Hz, |
| | | | | | 2H), 3.81 (s, 3H). |
| 86 | 62 | 395.46 | N-[(2- | HPLC | 1H NMR (400 MHz, DMSO): δ |
| | | | methoxypyridin-3- | 95.5%, | 8.61 (d, J = 1.9 Hz, 1H), 8.46 (d, |
| | | | yl)methyl]-8-(1- | m/z = 396.3 | J = 1.9 Hz, 1H), 8.10 (dd, J = 5.0, |
| | | | methyl-1H- | [M + H]⁺ | 1.8 Hz, 1H), 7.68 (dd, J = 7.3, |
| | | | indol-6-yl)quinoxalin- | | 1.8 Hz, 1H), 7.62 (s, 1H), |
| | | | 6-amine | | 7.61 (d, J = 8.2 Hz, 1H), 7.45 (d, |
| | | | | | J = 2.6 Hz, 1H), 7.39 (d, J = 3.0 Hz, |
| | | | | | 1H), 7.26 (dd, J = 8.1, 1.4 Hz, |
| | | | | | 1H), 7.21 (t, J = 6.0 Hz, 1H), |
| | | | | | 6.98 (dd, J = 7.3, 5.0 Hz, 1H), |
| | | | | | 6.68 (d, J = 2.6 Hz, 1H), |
| | | | | | 6.48 (dd, J = 3.1, 0.7 Hz, 1H), |
| | | | | | 4.42 (d, J = 5.8 Hz, 2H), 3.98 (s, 3H), |
| | | | | | 3.82 (s, 3H). |
| 87 | 63 | 381.43 | 3-({[8-(1-methyl-1H- | HPLC | 1H NMR (400 MHz, DMSO): δ |
| | | | indol-6-yl)quinoxalin- | 97.9%, | 11.72 (s, 1H), 8.61 (d, J = 1.9 Hz, |
| | | | 6-yl]amino}methyl)- | m/z = 382.2 | 1H), 8.45 (d, J = 1.9 Hz, 1H), |
| | | | 1,2-dihydropyridin-2- | [M + H]⁺ | 7.64-7.62 (m, 1H), |
| | | | one | | 7.62-7.58 (m, 1H), 7.43 (dd, J = 6.8, |
| | | | | | 2.3 Hz, 2H), 7.39 (d, J = 3.0 Hz, 1H), |
| | | | | | 7.32 (dd, J = 6.5, 2.0 Hz, 1H), |
| | | | | | 7.27 (dd, J = 8.1, 1.5 Hz, 1H), |
| | | | | | 7.10 (t, J = 5.9 Hz, 1H), 6.69 (d, |
| | | | | | J = 2.6 Hz, 1H), 6.48 (dd, J = 3.1, |
| | | | | | 0.8 Hz, 1H), 6.18 (t, J = 6.6 Hz, |
| | | | | | 1H), 4.24 (d, J = 5.8 Hz, 2H), |
| | | | | | 3.82 (s, 3H). |
| 88 | 37 | 407.47 | 4-({[8-(1-methyl-1H- | HPLC | 1H NMR (400 MHz, DMSO) δ |
| | | | indol-6-yl)quinoxalin- | 92.8%, | 8.59 (d, J = 1.9 Hz, 1H), 8.44 (d, |
| | | | 6- | m/z = 408.3 | J = 1.9 Hz, 1H), 7.92 (s, 1H), |
| | | | yl]amino}methyl)benzamide | [M + H]⁺ | 7.86 (d, J = 8.3 Hz, 2H), 7.60 (d, |
| | | | | | J = 8.1 Hz, 2H), 7.51 (d, J = 8.3 Hz, |
| | | | | | 2H), 7.44 (d, J = 2.6 Hz, 1H), |
| | | | | | 7.38 (d, J = 3.1 Hz, 1H), 7.35 (t, |
| | | | | | J = 5.9 Hz, 1H), 7.31 (s, 1H), |
| | | | | | 7.26 (dd, J = 8.2, 1.4 Hz, 1H), |
| | | | | | 6.72 (d, J = 2.6 Hz, 1H), 6.47 (d, |
| | | | | | J = 2.9 Hz, 1H), 4.55 (d, J = 5.8 Hz, |
| | | | | | 2H), 3.81 (s, 3H). |
| 89 | 38 | 432.48 | 8-(1-methyl-1H- | HPLC | 1H NMR (400 MHz, DMSO) δ |
| | | | indol-6-yl)-N-{[4-(1H- | 93.8%, | 8.59 (d, J = 1.9 Hz, 1H), 8.44 (d, |
| | | | 1,2,3,4- | m/z = 433.3 | J = 1.9 Hz, 1H), 8.01 (d, J = 8.3 Hz, |
| | | | tetrazol-5- | [M + H]⁺ | 2H), 7.64-7.55 (m, J = 8.5, |
| | | | yl)phenyl]methyl}quinoxalin- | | 8.0 Hz, 4H), 7.46 (d, J = 2.6 Hz, |
| | | | 6-amine | | 1H), 7.38 (d, J = 3.0 Hz, 1H), |
| | | | | | 7.35 (t, J = 5.9 Hz, 1H), |
| | | | | | 7.27 (dd, J = 8.1, 1.5 Hz, 1H), |
| | | | | | 6.77 (d, J = 2.6 Hz, 1H), 6.47 (dd, J = 3.0, |
| | | | | | 0.8 Hz, 1H), 4.55 (d, J = 5.8 Hz, |
| | | | | | 2H), 3.81 (s, 3H). |
| 90 | 34 | 379.46 | N-methyl-8-(1- | HPLC | 1H NMR (400 MHz, DMSO) δ |
| | | | methyl-1H-indol-6- | 95.6%, | 8.68 (d, J = 1.8 Hz, 1H), 8.56 (d, |
| | | | yl)-N-(pyridin-3- | m/z = 380.3 | J = 1.6 Hz, 1H), 8.52 (d, J = 1.8 Hz, |
| | | | ylmethyl)quinoxalin- | [M + H]⁺ | 1H), 8.47 (dd, J = 4.7, 1.5 Hz, |
| | | | 6-amine | | 1H), 7.69 (d, J = 8.1 Hz, 1H), |
| | | | | | 7.62-7.50 (m, 3H), |
| | | | | | 7.41-7.34 (m, 2H), 7.28 (dd, J = 8.1, 1.5 Hz, |
| | | | | | 1H), 7.04 (d, J = 2.9 Hz, 1H), |
| | | | | | 6.46 (dd, J = 3.1, 0.8 Hz, 1H), |
| | | | | | 4.92 (s, 2H), 3.82 (d, J = 9.3 Hz, |
| | | | | | 3H), 3.30 (s, J = 16.3 Hz, 3H). |
| 91 | 53 | 405.49 | 8-(1-methyl-1H- | HPLC | 1H NMR (400 MHz, DMSO): δ |
| | | | indol-6-yl)-N-[(8S)- | 98.7%, | 8.65 (d, J = 1.9 Hz, 1H), 8.52 (s, |
| | | | 5,6,7,8- | m/z = 406.2 | 1H), 8.47 (d, J = 1.9 Hz, 1H), |
| | | | tetrahydroisoquinolin- | [M + H]⁺ | 8.34 (d, J = 5.1 Hz, 1H), |
| | | | 8-yl]quinoxalin-6- | | 7.65-7.62 (m, 1H), 7.60 (dd, J = 8.2, |
| | | | amine | | 0.6 Hz, 1H), 7.44 (d, J = 2.6 Hz, |
| | | | | | 1H), 7.38 (d, J = 3.1 Hz, 1H), |
| | | | | | 7.27 (dd, J = 8.2, 1.5 Hz, 1H), |
| | | | | | 7.19 (d, J = 5.2 Hz, 1H), |
| | | | | | 7.09-7.04 (m, 2H), 6.47 (dd, J = 3.1, |
| | | | | | 0.8 Hz, 1H), 5.02-4.94 (m, |
| | | | | | 1H), 3.81 (s, 3H), |
| | | | | | 2.89-2.72 (m, 2H), 2.05-1.96 (m, 2H), |
| | | | | | 1.94-1.79 (m, 2H). |
| 92 | 54 | 405.49 | 8-(1-methyl-1H- | HPLC | 1H NMR (400 MHz, DMSO): δ |
| | | | indol-6-yl)-N-[(8R)- | 97.0%, | 8.65 (d, J = 1.9 Hz, 1H), 8.52 (s, |
| | | | 5,6,7,8- | m/z = 406.2 | 1H), 8.47 (d, J = 1.9 Hz, 1H), |
| | | | tetrahydroisoquinolin- | [M + H]⁺ | 8.34 (d, J = 5.1 Hz, 1H), 7.63 (d, |
| | | | 8-yl]quinoxalin-6- | | J = 0.6 Hz, 1H), 7.60 (dd, J = 8.2, |
| | | | amine | | 0.4 Hz, 1H), 7.44 (d, J = 2.6 Hz, |
| | | | | | 1H), 7.38 (d, J = 3.1 Hz, 1H), |
| | | | | | 7.27 (dd, J = 8.2, 1.4 Hz, 1H), |
| | | | | | 7.18 (d, J = 5.1 Hz, 1H), |
| | | | | | 7.10-7.03 (m, 2H), 6.47 (dd, J = 3.1, |
| | | | | | 0.8 Hz, 1H), 4.98 (dt, J = 8.8, |
| | | | | | 5.2 Hz, 1H), 3.81 (s, 3H), |
| | | | | | 2.89-2.71 (m, 2H), 2.05-1.95 (m, |
| | | | | | 2H), 1.95-1.79 (m, 2H). |
| 93 | 104 | 379.46 | 8-(1-methyl-1H- | HPLC | 1H NMR (400 MHz, DMSO) δ |
| | | | indol-4-yl)-N-[1- | 98.3%, | 8.72 (d, J = 1.8 Hz, 1H), 8.56 (d, |
| | | | (pyridin-3- | m/z = 380.3 | J = 1.9 Hz, 1H), 8.46 (dd, J = 4.7, |
| | | | yl)ethyl]quinoxalin-6- | [M + H]⁺ | 1.6 Hz, 1H), 8.34 (d, J = 1.9 Hz, |
| | | | amine | | 1H), 7.86 (dt, J = 7.9, 2.0 Hz, |
| | | | | | 1H), 7.47 (dd, J = 5.3, 4.3 Hz, |
| | | | | | 2H), 7.38 (ddd, J = 7.9, 4.8, |
| | | | | | 0.7 Hz, 1H), 7.29-7.25 (m, |
| | | | | | 2H), 7.25-7.20 (m, 1H), |
| | | | | | 7.11 (dd, J = 7.2, 0.9 Hz, 1H), |
| | | | | | 6.67 (d, J = 2.6 Hz, 1H), 5.95 (d, J = 2.4 Hz, |
| | | | | | 1H), 4.82 (p, J = 6.7 Hz, |
| | | | | | 1H), 3.84 (s, 3H), 1.55 (d, J = 6.8 Hz, |
| | | | | | 3H). |
| 94 | 45 | 371.44 | 4-{[8-(1-methyl-1H- | HPLC | 1H NMR (400 MHz, DMSO) δ |
| | | | indol-6-yl)quinoxalin- | 93.3%, | 8.64 (d, J = 1.9 Hz, 1H), 8.47 (d, |
| | | | 6- | m/z = 372.8 | J = 1.9 Hz, 1H), 7.66-7.57 (m, |
| | | | yl]amino}piperidin-2- | [M + H]⁺ | 3H), 7.39 (t, J = 2.8 Hz, 2H), |
| | | | one | | 7.27 (dd, J = 8.2, 1.5 Hz, 1H), |
| | | | | | 6.89 (d, J = 2.5 Hz, 1H), 6.71 (d, |
| | | | | | J = 7.4 Hz, 1H), 6.47 (dd, J = 3.1, |
| | | | | | 0.8 Hz, 1H), 3.99 (s, 1H), |
| | | | | | 3.82 (s, 3H), 3.28 (t, J = 5.0 Hz, |
| | | | | | 2H), 2.63 (dd, J = 17.2, 4.5 Hz, |
| | | | | | 1H), 2.13 (d, J = 8.7 Hz, 1H), |
| | | | | | 1.69 (dt, J = 15.8, 7.0 Hz, 2H). |
| 95 | 71 | 431.49 | 8-(1-methyl-1H- | HPLC | 1H NMR (400 MHz, DMSO) δ |
| | | | indol-6-yl)-N-{[5-(1H- | 97.7%, | 13.09 (s, 1H), 8.80 (d, J = 1.9 Hz, |
| | | | pyrazol-4- | m/z = 432.3 | 1H), 8.62 (d, J = 1.8 Hz, 1H), |
| | | | yl)pyridin-3- | [M + H]⁺ | 8.48 (dd, J = 13.1, 1.7 Hz, 2H), |
| | | | yl]methyl}quinoxalin- | | 8.32 (s, 1H), 8.09 (s, 1H), |
| | | | 6-amine | | 8.03 (s, 1H), 7.65-7.56 (m, 2H), |
| | | | | | 7.46 (d, J = 2.5 Hz, 1H), 7.38 (d, |
| | | | | | J = 3.0 Hz, 1H), 7.32-7.22 (m, |
| | | | | | 2H), 6.85 (d, J = 2.4 Hz, 1H), |
| | | | | | 6.47 (d, J = 2.7 Hz, 1H), 4.53 (d, |
| | | | | | J = 5.5 Hz, 2H), 3.81 (s, 3H). |
| 96 | Intermediate | 444.33 | N-[(5-bromopyridin- | HPLC | 1H NMR (400 MHz, DMSO) δ |
| | 24 | | 3-yl)methyl]-8-(1- | 87.2%, | 8.68 (s, 1H), 8.62 (t, J = 2.3 Hz, |
| | | | methyl-1H- | m/z = 444.5 | 2H), 8.48 (d, J = 1.9 Hz, 1H), |
| | | | indol-6-yl)quinoxalin- | [M + H]⁺ | 8.12 (s, 1H), 7.67 z 7.56 (m, |
| | | | 6-amine | | 2H), 7.43 (d, J = 2.6 Hz, 1H), |
| | | | | | 7.39 (d, J = 3.1 Hz, 1H), 7.31 (t, |
| | | | | | J = 6.9 Hz, 1H), 7.27 (dd, J = 8.1, |
| | | | | | 1.4 Hz, 1H), 6.82 (d, J = 2.5 Hz, |
| | | | | | 1H), 6.47 (dd, J = 3.0, 0.6 Hz, |
| | | | | | 1H), 4.56 (d, J = 5.9 Hz, 2H), |
| | | | | | 3.82 (s, 3H). |
| 97 | 42 | 357.45 | 8-(1-methyl-1H- | HPLC | 1H NMR (400 MHz, DMSO) δ |
| | | | indol-6-yl)-N- | 98.8%, | 8.75 (d, J = 1.9 Hz, 1H), 8.61 (d, |
| | | | (piperidin-4- | m/z = 358.1 | J = 1.9 Hz, 1H), 7.71 (d, J = 2.8 Hz, |
| | | | yl)quinoxalin-6- | [M + H]⁺ | 1H), 7.67 (s, 1H), 7.61 (s, |
| | | | amine | | 1H), 7.59 (s, 1H), 7.39 (d, J = 3.0 Hz, |
| | | | | | 2H), 7.32 (d, J = 1.5 Hz, |
| | | | | | 1H), 7.29 (s, 1H), 7.28 (d, J = 2.8 Hz, |
| | | | | | 1H), 6.47 (d, J = 3.9 Hz, |
| | | | | | 1H), 4.12 (d, J = 12.8 Hz, 2H), |
| | | | | | 3.83 (s, 3H), 3.02 (t, J = 12.2 Hz, |
| | | | | | 2H), 2.05 (d, J = 11.7 Hz, |
| | | | | | 2H), 1.70 (d, J = 14.7 Hz, 2H). |
| 98 | 109 | 460.53 | 8-(3-methyl-1- | HPLC | 1H NMR (400 MHz, DMSO) δ |
| | | | benzofuran-5-yl)-N- | 96.3%, | 8.70 (d, J = 2.1 Hz, 1H), 8.58 (d, |
| | | | {1-[5-(1-methyl- | m/z = 461.2 | J = 1.9 Hz, 1H), 8.52 (d, J = 2.0 Hz, |
| | | | 1H-pyrazol-4- | [M + H]⁺ | 1H), 8.44 (d, J = 1.9 Hz, 1H), |
| | | | yl)pyridin-3- | | 8.23 (s, 1H), 8.05 (t, J = 2.0 Hz, |
| | | | yl]ethyl}quinoxalin- | | 1H), 7.94 (s, 1H), 7.83 (d, J = 1.3 Hz, |
| | | | 6- | | 1H), 7.74 (d, J = 1.4 Hz, |
| | | | amine | | 1H), 7.61 (d, J = 8.5 Hz, 1H), |
| | | | | | 7.52 (dd, J = 8.5, 1.7 Hz, 1H), |
| | | | | | 7.47 (d, J = 2.6 Hz, 1H), 7.25 (d, |
| | | | | | J = 6.8 Hz, 1H), 6.69 (d, J = 2.4 Hz, |
| | | | | | 1H), 4.86-4.78 (m, 1H), |
| | | | | | 3.87 (s, 3H), 2.23 (d, J = 1.2 Hz, |
| | | | | | 3H), 1.59 (d, J = 6.8 Hz, 3H). |
| 99 | 72 | 443.5 | 8-(1-methyl-1H- | HPLC | 1H NMR (400 MHz, DMSO) δ |
| | | | indol-6-yl)-N-{[5- | 95.1%, | 9.25 (s, 1H), 9.23 (s, 2H), |
| | | | (pyrimidin-5- | m/z = 444.2 | 8.96 (d, J = 2.2 Hz, 1H), 8.79 (d, J = 1.9 Hz, |
| | | | yl)pyridin-3- | [M + H]⁺ | 1H), 8.62 (d, J = 1.9 Hz, |
| | | | yl]methyl}quinoxalin- | | 1H), 8.47 (d, J = 1.9 Hz, 1H), |
| | | | 6-amine | | 8.37 (t, J = 2.0 Hz, 1H), 7.62 (s, |
| | | | | | 1H), 7.60 (d, J = 8.2 Hz, 1H), |
| | | | | | 7.46 (d, J = 2.6 Hz, 1H), 7.39 (d, |
| | | | | | J = 3.0 Hz, 1H), 7.33 (t, J = 5.7 Hz, |
| | | | | | 1H), 7.27 (dd, J = 8.2, 1.4 Hz, |
| | | | | | 1H), 6.88 (d, J = 2.6 Hz, 1H), |
| | | | | | 6.47 (dd, J = 3.0, 0.7 Hz, 1H), |
| | | | | | 4.63 (d, J = 5.7 Hz, 2H), 3.81 (s, |
| | | | | | 3H). |
| 100 | 75 | 380.45 | N-[(5-aminopyridin- | HPLC | 1H NMR (400 MHz, DMSO) δ |
| | | | 3-yl)methyl]-8-(1- | 99.8%, | 8.61 (d, J = 1.9 Hz, 1H), 8.45 (d, |
| | | | methyl-1H- | m/z = 381.4 | J = 1.9 Hz, 1H), 7.83 (dd, J = 7.4, |
| | | | indol-6-yl)quinoxalin- | [M + H]⁺ | 2.2 Hz, 2H), 7.62-7.58 (m, |
| | | | 6-amine | | 2H), 7.42 (d, J = 2.6 Hz, 1H), |
| | | | | | 7.39 (d, J = 3.0 Hz, 1H), |
| | | | | | 7.25 (dt, J = 11.8, 4.1 Hz, 2H), |
| | | | | | 6.96-6.93 (m, 1H), 6.76 (d, J = 2.6 Hz, |
| | | | | | 1H), 6.48 (dd, J = 3.1, 0.8 Hz, |
| | | | | | 1H), 5.32 (s, 2H), 4.37 (d, J = 5.8 Hz, |
| | | | | | 2H), 3.82 (s, 3H). |
| 101 | 73 | 431.49 | 8-(1-methyl-1H- | HPLC | 1H NMR (400 MHz, DMSO) δ |
| | | | indol-6-yl)-N-{[5-(1H- | 92.6%, | 8.93 (s, 1H), 8.61 (d, J = 1.8 Hz, |
| | | | pyrazol-5- | m/z = 432.1 | 2H), 8.46 (d, J = 1.9 Hz, 1H), |
| | | | yl)pyridin-3- | [M + H]⁺ | 8.27 (s, 1H), 7.84 (s, 1H), |
| | | | yl]methyl}quinoxalin- | | 7.62 (d, J = 5.0 Hz, 2H), 7.59 (s, 1H), |
| | | | 6-amine | | 7.46 (d, J = 2.6 Hz, 1H), 7.39 (d, |
| | | | | | J = 3.1 Hz, 1H), 7.27 (dd, J = 8.1, |
| | | | | | 1.5 Hz, 1H), 6.90 (s, 1H), |
| | | | | | 6.86-6.81 (m, 2H), 6.47 (dd, J = 3.1, |
| | | | | | 0.8 Hz, 1H), 4.58 (d, J = 5.9 Hz, |
| | | | | | 2H), 3.81 (s, 3H). |
| 102 | 107 | 359.42 | 8-(3-methyl-1- | HPLC | 1H NMR (400 MHz, DMSO) δ |
| | | | benzofuran-5-yl)-N- | 97.3%, | 8.64 (d, J = 1.9 Hz, 1H), 8.45 (d, |
| | | | (oxan-4- | m/z = 360.2 | J = 1.9 Hz, 1H), 7.83 (d, J = 1.3 Hz, |
| | | | yl)quinoxalin-6- | [M + H]⁺ | 1H), 7.76 (d, J = 1.4 Hz, 1H), |
| | | | amine | | 7.61 (d, J = 8.5 Hz, 1H), |
| | | | | | 7.52 (dd, J = 8.5, 1.7 Hz, 1H), 7.38 (t, |
| | | | | | J = 5.2 Hz, 1H), 6.90 (d, J = 2.4 Hz, |
| | | | | | 1H), 6.64 (d, J = 7.7 Hz, 1H), |
| | | | | | 3.91 (dd, J = 8.1, 3.3 Hz, 2H), |
| | | | | | 3.76-3.67 (m, 1H), |
| | | | | | 3.56-3.47 (m, 2H), 2.24 (d, J = 1.3 Hz, |
| | | | | | 3H), 2.00 (d, J = 10.8 Hz, |
| | | | | | 2H), 1.52-1.43 (m, 2H). |
| 103 | 43 | 399.49 | 1-(4-{[8-(1-methyl- | HPLC | 1H NMR (400 MHz, DMSO) δ |
| | | | 1H-indol-6- | 96.1%, | 8.63 (d, J = 1.9 Hz, 1H), 8.45 (d, |
| | | | yl)quinoxalin-6- | m/z = 400.4 | J = 1.9 Hz, 1H), 7.63 (s, 1H), |
| | | | yl]amino}piperidin-1- | [M + H]⁺ | 7.60 (d, J = 8.2 Hz, 1H), 7.38 (d, |
| | | | yl)ethan-1-one | | J = 3.1 Hz, 1H), 7.37 (d, J = 2.6 Hz, |
| | | | | | 1H), 7.27 (dd, J = 8.2, 1.4 Hz, |
| | | | | | 1H), 6.90 (d, J = 2.6 Hz, 1H), |
| | | | | | 6.62 (d, J = 7.8 Hz, 1H), |
| | | | | | 6.47 (dd, J = 3.1, 0.8 Hz, 1H), |
| | | | | | 4.27 (d, J = 12.2 Hz, 1H), |
| | | | | | 3.88-3.70 (m, 5H), 3.30-3.24 (m, 1H), |
| | | | | | 2.95-2.86 (m, 1H), |
| | | | | | 2.10-1.97 (m, 5H), 1.42 (d, J = 11.0 Hz, |
| | | | | | 1H), 1.31 (d, J = 10.1 Hz, |
| | | | | | 1H). |
| 104 | 46 | 397.5 | N-{7- | HPLC | 1H NMR (400 MHz, DMSO) δ |
| | | | azaspiro[3.5]nonan- | 87.6%, | 8.62 (t, J = 2.1 Hz, 1H), 8.44 (t, |
| | | | 1-yl}-8-(1-methyl-1H- | m/z = 398.3 | J = 1.9 Hz, 1H), 7.66-7.55 (m, |
| | | | indol-6- | [M + H]⁺ | 2H), 7.40 (dd, J = 7.2, 2.8 Hz, |
| | | | yl)quinoxalin-6- | | 2H), 7.27 (dd, J = 8.1, 1.5 Hz, |
| | | | amine | | 1H), 6.89-6.80 (m, 2H), |
| | | | | | 6.48 (dd, J = 3.0, 0.8 Hz, 1H), |
| | | | | | 3.97-3.84 (m, 1H), 3.82 (s, J = 4.3 Hz, |
| | | | | | 3H), 2.90-2.77 (m, 2H), |
| | | | | | 2.67 (dd, J = 11.6, 9.7 Hz, 1H), |
| | | | | | 2.38-2.22 (m, 2H), |
| | | | | | 2.06-1.89 (m, 2H), 1.77 (dd, J = 23.0, |
| | | | | | 10.9 Hz, 3H), 1.60-1.41 (m, |
| | | | | | 3H). |
| 105 | 57 | 448.5 | 8-(1-methyl-1H- | HPLC | 1H NMR (400 MHz, DMSO) δ |
| | | | indol-6-yl)-N- | 79.9%, | 8.70 (d, J = 1.7 Hz, 1H), 8.56 (d, |
| | | | [piperidin-4- | m/z = 449.0 | J = 1.9 Hz, 1H), 8.43 (dd, J = 4.8, |
| | | | yl(pyridin-3- | [M + H]⁺ | 1.6 Hz, 1H), 8.41 (d, J = 1.9 Hz, |
| | | | yl)methyl]quinoxalin- | | 1H), 7.86-7.82 (m, 1H), |
| | | | 6-amine | | 7.61-7.57 (m, 2H), 7.51 (d, J = 2.6 Hz, |
| | | | | | 1H), 7.38 (d, J = 3.1 Hz, |
| | | | | | 1H), 7.38-7.34 (m, 1H), |
| | | | | | 7.27-7.20 (m, 2H), 6.69 (d, J = 2.4 Hz, |
| | | | | | 1H), 6.47 (dd, J = 3.0, 0.8 Hz, |
| | | | | | 1H), 4.46 (t, J = 7.9 Hz, 1H), |
| | | | | | 3.82 (s, 3H), 2.95 (dd, J = 36.5, |
| | | | | | 11.5 Hz, 3H), 2.71-2.64 (m, |
| | | | | | 1H), 2.47-2.31 (m, 2H), |
| | | | | | 2.02-1.71 (m, 4H). |
| 106 | 74 | 450.5 | 8-(1-methyl-1H- | HPLC | — |
| | | | indol-6-yl)-N-{[5- | 91.2%, | |
| | | | (morpholin-4- | m/z = 451.1 | |
| | | | yl)pyridin-3- | [M + H]⁺ | |
| | | | yl]methyl}quinoxalin- | | |
| | | | 6-amine | | |
| 107 | 108 | 374.4 | 8-(3-methyl-1- | HPLC | 1H NMR (400 MHz, DMSO) δ |
| | | | benzofuran-5-yl)-N- | 92.1%, | 8.78 (d, J = 1.9 Hz, 1H), 8.63 (d, |
| | | | (morpholin-2- | m/z = 375.4 | J = 1.9 Hz, 1H), 7.83 (d, J = 1.3 Hz, |
| | | | ylmethyl)quinoxalin- | [M + H]⁺ | 1H), 7.81 (d, J = 1.3 Hz, 1H), |
| | | | 6-amine | | 7.75 (d, J = 2.8 Hz, 1H), |
| | | | | | 7.62 (dd, J = 8.5, 0.5 Hz, 1H), |
| | | | | | 7.55 (dd, J = 8.5, 1.7 Hz, 1H), |
| | | | | | 7.32 (d, J = 2.8 Hz, 1H), 6.90 (s, 1H), |
| | | | | | 4.07-3.95 (m, 2H), 3.89 (d, J = 11.8 Hz, |
| | | | | | 1H), 3.69 (td, J = 11.5, |
| | | | | | 2.6 Hz, 1H), 3.56-3.48 (m, 1H), |
| | | | | | 2.91 (td, J = 12.0, 3.5 Hz, 1H), |
| | | | | | 2.74 (t, J = 7.4 Hz, 1H), |
| | | | | | 2.71-2.67 (m, 1H), 2.62 (dd, J = 12.1, |
| | | | | | 10.6 Hz, 1H), 2.57-2.52 (m, |
| | | | | | 1H), 2.25 (d, J = 1.3 Hz, 3H). |
| 108 | 47 | 379.5 | 8-(1-methyl-1H- | HPLC | 1H NMR (400 MHz, DMSO) δ |
| | | | indol-6-yl)-N-[(4- | 95.5%, | 8.63 (d, J = 1.9 Hz, 1H), 8.51 (s, |
| | | | methylpyridin-3- | m/z = 380.2 | 1H), 8.46 (d, J = 1.9 Hz, 1H), |
| | | | yl)methyl]quinoxalin- | [M + H]⁺ | 8.37 (d, J = 4.9 Hz, 1H), |
| | | | 6-amine | | 7.64-7.62 (m, J = 0.6 Hz, 1H), |
| | | | | | 7.60 (dd, J = 8.2, 0.5 Hz, 1H), |
| | | | | | 7.45 (d, J = 2.6 Hz, 1H), 7.38 (d, J = 3.1 Hz, |
| | | | | | 1H), 7.27 (dd + d, J = 8.1, |
| | | | | | 5.0, 1.5 Hz, 2H), 7.11 (t, J = 5.5 Hz, |
| | | | | | 1H), 6.84 (d, J = 2.6 Hz, 1H), |
| | | | | | 6.47 (dd, J = 3.1, 0.8 Hz, 1H), |
| | | | | | 4.49 (d, J = 5.4 Hz, 2H), 3.81 (s, |
| | | | | | 3H), 2.42 (s, 3H). |
| 109 | 66 | 383.4 | N-[(4-fluoropyridin- | HPLC | 1H NMR (400 MHz, DMSO) δ |
| | | | 3-yl)methyl]-8-(1- | 98.4%, | 8.63 (d, J = 1.9 Hz, 1H), 8.48 (d, |
| | | | methyl-1H- | m/z = 384.3 | J = 1.9 Hz, 1H), 8.17 (d, J = 4.8 Hz, |
| | | | indol-6-yl)quinoxalin- | [M + H]⁺ | 1H), 8.02-7.97 (m, 1H), |
| | | | 6-amine | | 7.63-7.59 (m, 2H), 7.45 (d, J = 2.6 Hz, |
| | | | | | 1H), 7.39 (d, J = 3.1 Hz, |
| | | | | | 1H), 7.37-7.33 (m, 1H), |
| | | | | | 7.32-7.25 (m, 2H), 6.79 (d, J = 2.6 Hz, |
| | | | | | 1H), 6.48 (dd, J = 3.1, 0.8 Hz, |
| | | | | | 1H), 4.54 (d, J = 5.9 Hz, 2H), |
| | | | | | 3.82 (s, 3H). |
| 110 | 68 | 381.4 | 5-({[8-(1-methyl-1H- | HPLC | 1H NMR (400 MHz, DMSO) δ |
| | | | indol-6-yl)quinoxalin- | 98.9, | 9.89 (s, 1H), 8.61 (d, J = 1.9 Hz, |
| | | | 6- | m/z = 382.3 | 1H), 8.46 (d, J = 1.9 Hz, 1H), |
| | | | yl]amino}methyl)pyridin- | [M + H]⁺ | 8.15 (d, J = 1.7 Hz, 1H), 8.02 (d, |
| | | | 3-ol | | J = 2.7 Hz, 1H), 7.60 (d, J = 7.9 Hz, |
| | | | | | 2H), 7.43 (d, J = 2.6 Hz, 1H), |
| | | | | | 7.39 (d, J = 3.0 Hz, 1H), 7.29 (d, |
| | | | | | J = 5.9 Hz, 1H), 7.26 (dd, J = 8.1, |
| | | | | | 1.5 Hz, 1H), |
| | | | | | 7.23-7.18 (m, 1H), 6.78 (d, J = 2.6 Hz, |
| | | | | | 1H), 6.48 (dd, J = 3.0, 0.7 Hz, |
| | | | | | 1H), 4.47 (d, J = 5.8 Hz, 2H), |
| | | | | | 3.82 (s, 3H). |
| 111 | 93 | 405.5 | 3-(7-{[1-(pyridin-3- | HPLC | 1H NMR (400 MHz, DMSO) δ |
| | | | yl)ethyl]amino}quinoxalin- | 99.3%, | 8.72 (d, J = 1.9 Hz, 1H), 8.61 (d, |
| | | | 5- | m/z = 406.1 | J = 1.9 Hz, 1H), 8.44 (dd, J = 5.6, |
| | | | yl)benzene-1- | [M + H]⁺ | 1.8 Hz, 2H), 8.03 (t, J = 1.6 Hz, |
| | | | sulfonamide | | 1H), 7.88 (ddd, J = 7.8, 1.8, |
| | | | | | 1.2 Hz, 1H), 7.85 (dt, J = 7.9, |
| | | | | | 1.9 Hz, 1H), 7.82 (ddd, J = 8.2, |
| | | | | | 1.6, 1.2 Hz, 1H), 7.67 (t, J = 7.9 Hz, |
| | | | | | 1H), 7.47 (d, J = 2.6 Hz, 1H), |
| | | | | | 7.41 (s, 2H), 7.39-7.33 (m, |
| | | | | | 2H), 6.67 (d, J = 2.5 Hz, 1H), |
| | | | | | 4.83 (p, J = 6.7 Hz, 1H), 1.55 (d, |
| | | | | | J = 6.8 Hz, 3H). |
| 112 | 55 | 406.5 | 8-(1-methyl-1H- | HPLC | 1H NMR (400 MHz, DMSO) δ |
| | | | indol-6-yl)-N- | 99.3%, | 8.64 (d, J = 1.9 Hz, 1H), 8.51 (d, |
| | | | (5,6,7,8- | m/z = 407.2 | J = 2.5 Hz, 1H), 8.49 (d, J = 2.4 Hz, |
| | | | tetrahydroquinoxalin- | [M + H]⁺ | 1H), 8.46 (d, J = 1.9 Hz, 1H), |
| | | | 5-yl)quinoxalin-6- | | 7.62 (s, 1H), 7.59 (d, J = 8.2 Hz, |
| | | | amine | | 1H), 7.42 (d, J = 2.6 Hz, 1H), |
| | | | | | 7.38 (d, J = 3.1 Hz, 1H), |
| | | | | | 7.27 (dd, J = 8.2, 1.5 Hz, 1H), |
| | | | | | 7.08 (d, J = 2.5 Hz, 1H), 7.05 (d, J = 7.9 Hz, |
| | | | | | 1H), 6.47 (dd, J = 3.0, |
| | | | | | 0.8 Hz, 1H), 4.98 (d, J = 7.4 Hz, |
| | | | | | 1H), 3.81 (s, 3H), 2.98 (dd, J = 13.7, |
| | | | | | 6.7 Hz, 2H), |
| | | | | | 2.22-2.01 (m, 4H) |
| 113 | 111 | 459.6 | 8-(3-methyl-1- | HPLC | 1H NMR (400 MHz, DMSO) δ |
| | | | benzofuran-5-yl)-N- | 94.1%, | 8.56 (d, J = 1.9 Hz, 1H), 8.42 (d, |
| | | | [(1S)-1-[3-(1- | m/z = 460.2 | J = 1.9 Hz, 1H), 8.10 (s, 1H), |
| | | | methyl-1H-pyrazol-4- | [M + H]⁺ | 7.84 (d, J = 0.8 Hz, 1H), 7.82 (d, |
| | | | yl)phenyl]ethyl]quinoxalin- | | J = 1.3 Hz, 1H), 7.73 (d, J = 1.4 Hz, |
| | | | 6-amine | | 1H), 7.69 (s, 1H), 7.60 (dd, J = 8.5, |
| | | | | | 0.5 Hz, 1H), 7.51 (dd, J = 8.5, |
| | | | | | 1.7 Hz, 1H), 7.47 (d, J = 2.6 Hz, |
| | | | | | 1H), 7.41 (dt, J = 7.1, 1.7 Hz, |
| | | | | | 1H), 7.35-7.26 (m, 2H), |
| | | | | | 7.22 (d, J = 6.6 Hz, 1H), 6.66 (d, |
| | | | | | J = 2.5 Hz, 1H), 4.70 (p, J = 6.5 Hz, |
| | | | | | 1H), 3.86 (s, 3H), 2.23 (d, J = 1.3 Hz, |
| | | | | | 3H), 1.55 (d, J = 6.7 Hz, |
| | | | | | 3H). |
| 114 | 105 | 377.5 | N-[1-(pyridin-3- | HPLC | 1H NMR (400 MHz, DMSO) δ |
| | | | yl)ethyl]-8-(quinolin- | 94.3%, | 8.95 (dd, J = 4.2, 1.7 Hz, 1H), |
| | | | 6- | m/z = 378.2 | 8.74 (d, J = 1.9 Hz, 1H), 8.62 (d, |
| | | | yl)quinoxalin-6- | [M + H]⁺ | J = 1.9 Hz, 1H), 8.47 (d, J = 1.9 Hz, |
| | | | amine | | 1H), 8.46-8.42 (m, 2H), |
| | | | | | 8.17 (d, J = 1.9 Hz, 1H), 8.09 (d, |
| | | | | | J = 8.7 Hz, 1H), 8.00 (dd, J = 8.7, |
| | | | | | 2.0 Hz, 1H), 7.87 (dt, J = 7.9, |
| | | | | | 1.9 Hz, 1H), 7.59 (dd, J = 8.3, |
| | | | | | 4.2 Hz, 1H), 7.56 (d, J = 2.6 Hz, |
| | | | | | 1H), 7.40-7.35 (m, 1H), |
| | | | | | 7.34 (d, J = 6.9 Hz, 1H), 6.69 (d, |
| | | | | | J = 2.5 Hz, 1H), 4.85 (p, J = 6.8 Hz, |
| | | | | | 1H), 1.56 (d, J = 6.8 Hz, 3H). |
| 115 | 56 | 449.6 | 8-(1-methyl-1H- | HPLC | 1H NMR (400 MHz, DMSO) δ |
| | | | indol-6-yl)-N-[oxan- | 97.8%, | 8.73 (s, 1H), 8.57 (d, J = 1.7 Hz, |
| | | | 4-yl(pyridin-3- | m/z = 450.4 | 1H), 8.45 (s, 1H), 8.42 (d, J = 1.8 Hz, |
| | | | yl)methyl]quinoxalin- | [M + H]⁺ | 1H), 7.88 (d, J = 7.9 Hz, |
| | | | 6-amine | | 1H), 7.60 (d, J = 2.0 Hz, 1H), |
| | | | | | 7.58 (s, 1H), 7.50 (d, J = 2.5 Hz, |
| | | | | | 1H), 7.41-7.36 (m, 2H), |
| | | | | | 7.26 (t, J = 8.1 Hz, 2H), 6.71 (d, J = 2.1 Hz, |
| | | | | | 1H), 6.47 (d, J = 2.8 Hz, |
| | | | | | 1H), 5.37 (dd, J = 6.0, 2.0 Hz, |
| | | | | | 1H), 4.52 (t, J = 7.8 Hz, 1H), |
| | | | | | 3.93 (d, J = 8.0 Hz, 1H), 3.82 (s, |
| | | | | | 3H), 3.80-3.75 (m, 1H), |
| | | | | | 3.22 (t, J = 11.1 Hz, 2H), 1.96 (d, J = 9.9 Hz, |
| | | | | | 2H), 1.41 (dd, J = 22.3, |
| | | | | | 8.9 Hz, 2H). |
| 116 | 50 | 385.5 | 8-(1-methyl-1H- | HPLC | 1H NMR (400 MHz, DMSO) δ |
| | | | indol-6-yl)-N-[(1- | 97.5%, | 8.62 (d, J = 1.9 Hz, 1H), 8.44 (d, |
| | | | methylpiperidin-2- | m/z = 386.4 | J = 1.9 Hz, 1H), 7.64 (s, 1H), |
| | | | yl)methyl]quinoxalin- | [M + H]⁺ | 7.61-7.58 (m, 1H), 7.50 (d, J = 2.6 Hz, |
| | | | 6-amine | | 1H), 7.38 (d, J = 3.0 Hz, |
| | | | | | 1H), 7.28 (dd, J = 8.2, 1.5 Hz, |
| | | | | | 1H), 6.78 (d, J = 2.6 Hz, 1H), |
| | | | | | 6.47 (dt, J = 6.5, 3.2 Hz, 2H), |
| | | | | | 3.82 (s, J = 4.8 Hz, 3H), |
| | | | | | 3.42-3.35 (m, 1H), 3.28-3.14 (m, |
| | | | | | 1H), 2.83 (d, J = 11.4 Hz, 1H), |
| | | | | | 2.29 (s, 3H), 2.07 (t, J = 9.7 Hz, |
| | | | | | 1H), 1.73 (dt, J = 11.2, 5.6 Hz, |
| | | | | | 2H), 1.59-1.40 (m, 3H), |
| | | | | | 1.27 (t, J = 9.9 Hz, 2H). |
| 117 | 64 | 381.4 | 5-({[8-(1-methyl-1H- | HPLC | 1H NMR (400 MHz, DMSO) δ |
| | | | indol-6-yl)quinoxalin- | 97.7%, | 11.50 (s, 1H), 8.63 (d, J = 1.9 Hz, |
| | | | 6-yl]amino}methyl)- | m/z = 382.2 | 1H), 8.46 (d, J = 1.9 Hz, 1H), |
| | | | 1,2-dihydropyridin-2- | [M + H]⁺ | 7.60 (d, J = 8.3 Hz, 2H), |
| | | | one | | 7.52 (dd, J = 9.4, 2.6 Hz, 1H), |
| | | | | | 7.45 (s, 1H), 7.39 (t, J = 2.8 Hz, 2H), |
| | | | | | 7.25 (dd, J = 8.1, 1.5 Hz, 1H), |
| | | | | | 7.05 (t, J = 5.7 Hz, 1H), 6.85 (d, |
| | | | | | J = 2.6 Hz, 1H), 6.47 (dd, J = 3.0, |
| | | | | | 0.7 Hz, 1H), 6.35 (d, J = 9.4 Hz, |
| | | | | | 1H), 4.21 (d, J = 5.6 Hz, 2H), |
| | | | | | 3.81 (s, 3H). |
| 118 | 106 | 377.5 | N-[1-(pyridin-3- | HPLC | 1H NMR (400 MHz, DMSO) δ |
| | | | yl)ethyl]-8-(quinolin- | 83.6%, | 8.95 (dd, J = 4.2, 1.7 Hz, 1H), |
| | | | 7- | m/z = 378.1 | 8.73 (d, J = 1.8 Hz, 1H), 8.62 (d, |
| | | | yl)quinoxalin-6- | [M + H]⁺ | J = 1.9 Hz, 1H), 8.47 (d, J = 1.9 Hz, |
| | | | amine | | 1H), 8.44 (td, J = 4.9, 1.4 Hz, |
| | | | | | 2H), 8.20 (t, J = 0.8 Hz, 1H), |
| | | | | | 8.05 (d, J = 8.4 Hz, 1H), |
| | | | | | 7.86 (ddd, J = 10.2, 6.1, 1.7 Hz, 2H), |
| | | | | | 7.61 z 7.54 (m, 2H), 7.37 (dd, J = 7.8, |
| | | | | | 4.9 Hz, 1H), 7.33 (d, J = 6.8 Hz, |
| | | | | | 1H), 6.70 (d, J = 2.4 Hz, |
| | | | | | 1H), 4.89 z 4.81 (m, 1H), |
| | | | | | 1.57 (d, J = 6.8 Hz, 3H). |
| 119 | 13 | 407.5 | 8-(1-methyl-1H- | HPLC | 1H NMR (400 MHz, DMSO) δ |
| | | | indol-6-yl)-N- | 99.6%, | 8.66 (d, J = 1.9 Hz, 1H), 8.47 (d, |
| | | | {2H,3H,4H- | m/z = 408.3 | J = 1.9 Hz, 1H), 8.18 (dd, J = 3.9, |
| | | | pyrano[3,2- | [M + H]⁺ | 2.0 Hz, 1H), |
| | | | b]pyridin-4- | | 7.65-7.55 (m, 2H), 7.46 (d, J = 2.6 Hz, |
| | | | yl}quinoxalin-6- | | 1H), 7.38 (d, J = 3.1 Hz, 1H), |
| | | | amine | | 7.32-7.25 (m, 3H), 7.12 (d, J = 2.5 Hz, |
| | | | | | 2H), 6.47 (dd, J = 3.1, |
| | | | | | 0.8 Hz, 1H), 4.92 (dd, J = 11.7, |
| | | | | | 4.7 Hz, 1H), 4.32 (ddd, J = 20.9, |
| | | | | | 11.0, 4.6 Hz, 2H), 3.81 (s, 3H), |
| | | | | | 2.38-2.26 (m, 1H), |
| | | | | | 2.26-2.12 (m, 1H). |
| 120 | 82 | 413.5 | 1-[2-({[8-(1-methyl- | HPLC | — |
| | | | 1H-indol-5- | 92.6%, | |
| | | | yl)quinoxalin-6- | m/z = 414.2 | |
| | | | yl]amino}methyl)piperidin- | [M + H]⁺ | |
| | | | 1-yl]ethan-1- | | |
| | | | one | | |
| 121 | 65 | 381.4 | N-[(2- | HPLC | 1H NMR (400 MHz, DMSO) δ |
| | | | aminopyrimidin-5- | 96.5%, | 8.63 (d, J = 1.9 Hz, 1H), 8.46 (d, |
| | | | yl)methyl]-8-(1- | m/z = 382.2 | J = 1.9 Hz, 1H), 8.33 (s, 2H), |
| | | | methyl-1H- | [M + H]⁺ | 7.62-7.60 (m, 1H), 7.59 (d, J = 8.6 Hz, |
| | | | indol-6-yl)quinoxalin- | | 1H), 7.39 (d, J = 2.7 Hz, |
| | | | 6-amine | | 1H), 7.38 (d, J = 3.1 Hz, 1H), |
| | | | | | 7.25 (dd, J = 8.1, 1.4 Hz, 1H), |
| | | | | | 7.04 (t, J = 5.5 Hz, 1H), 6.86 (d, |
| | | | | | J = 2.5 Hz, 1H), 6.57 (s, 2H), |
| | | | | | 6.47 (d, J = 3.0 Hz, 1H), 4.26 (d, |
| | | | | | J = 5.4 Hz, 2H), 3.81 (s, 3H). |
| 122 | 110 | 446.5 | 8-(3-methyl-1- | HPLC | 1H NMR (400 MHz, DMSO) δ |
| | | | benzofuran-5-yl)-N- | 93.8%, | 8.74 (d, J = 2.1 Hz, 1H), 8.63 (d, |
| | | | {[5-(1-methyl-1H- | m/z = 447.3 | J = 1.9 Hz, 1H), 8.50 (d, J = 2.0 Hz, |
| | | | pyrazol-4-yl)pyridin- | [M + H]⁺ | 1H), 8.47 (d, J = 1.9 Hz, 1H), |
| | | | 3- | | 8.25 (s, 1H), 8.03 (t, J = 2.1 Hz, |
| | | | yl]methyl}quinoxalin- | | 1H), 7.95 (d, J = 0.8 Hz, 1H), |
| | | | 6-amine | | 7.83 (d, J = 1.3 Hz, 1H), 7.76 (d, |
| | | | | | J = 1.3 Hz, 1H), 7.64-7.59 (m, |
| | | | | | 1H), 7.52 (dd, J = 8.5, 1.7 Hz, |
| | | | | | 1H), 7.45 (d, J = 2.6 Hz, 1H), |
| | | | | | 7.29 (t, J = 5.7 Hz, 1H), 6.86 (d, |
| | | | | | J = 2.6 Hz, 1H), 4.53 (d, J = 5.8 Hz, |
| | | | | | 2H), 3.88 (s, 3H), 2.23 (d, J = 1.3 Hz, |
| | | | | | 3H). |
| 123 | 58 | 490.6 | 1-[4-({[8-(1-methyl- | HPLC | 1H NMR (400 MHz, DMSO) δ |
| | | | 1H-indol-6- | 93.2%, | 8.71 (d, J = 1.8 Hz, 1H), 8.57 (d, |
| | | | yl)quinoxalin-6- | m/z = 491.2 | J = 1.4 Hz, 1H), 8.45 (dd, J = 4.7, |
| | | | yl]amino}(pyridin-3- | [M + H]⁺ | 1.6 Hz, 1H), 8.42 (d, J = 1.9 Hz, |
| | | | yl)methyl)piperidin- | | 1H), 7.86 (d, J = 7.9 Hz, 1H), |
| | | | 1-yl]ethan-1- | | 7.60 (s, 1H), 7.58 (s, 1H), |
| | | | one | | 7.51 (t, J = 2.3 Hz, 1H), 7.38 (dd, J = 6.8, |
| | | | | | 3.9 Hz, 2H), 7.25 (dd, J = 8.2, |
| | | | | | 1.4 Hz, 2H), 6.70 (s, 1H), |
| | | | | | 6.47 (dd, J = 3.1, 0.6 Hz, 1H), |
| | | | | | 4.56 (d, J = 6.6 Hz, 1H), 4.46 (d, |
| | | | | | J = 13.3 Hz, 1H), 4.37 (d, J = 13.6 Hz, |
| | | | | | 1H), 3.90 (d, J = 14.5 Hz, |
| | | | | | 2H), 3.82 (s, 3H), 2.99 (t, J = 21.1 Hz, |
| | | | | | 2H), 2.43 (d, J = 12.8 Hz, |
| | | | | | 1H), 2.01 (s, 1H), 1.99 (s, |
| | | | | | 2H), 1.98 (d, J = 6.3 Hz, 3H). |
| 124 | 67 | 400.9 | N-[(2- | HPLC | 1H NMR (400 MHz, DMSO) δ |
| | | | chloropyrimidin-5- | 92.2%, | 8.87 (s, 2H), 8.64 (d, J = 1.9 Hz, |
| | | | yl)methyl]-8-(1- | m/z = 401.2 | 1H), 8.49 (d, J = 1.9 Hz, 1H), |
| | | | methyl-1H- | [M + H]⁺ | 7.63-7.62 (m, 1H), 7.60 (dd, J = 8.2 Hz, |
| | | | indol-6-yl)quinoxalin- | | 1H), 7.41 (d, J = 2.6 Hz, |
| | | | 6-amine | | 1H), 7.39 (d, J = 3.1 Hz, 1H), |
| | | | | | 7.27 (dd + t, J = 8.2, 6.0, 1.5 Hz, |
| | | | | | 2H), 6.86 (d, J = 2.6 Hz, 1H), |
| | | | | | 6.47 (dd, J = 3.1, 0.8 Hz, 1H), |
| | | | | | 4.58 (d, J = 5.9 Hz, 2H), 3.81 (s, |
| | | | | | 3H). |
| 125 | 48 | 387.5 | 8-(1-methyl-1H- | HPLC | 1H NMR (400 MHz, DMSO) δ |
| | | | indol-6-yl)-N-[(4- | 99.4%, | 8.63 (d, J = 1.9 Hz, 1H), 8.45 (d, |
| | | | methylmorpholin-2- | m/z = 388.3 | J = 1.9 Hz, 1H), 7.62 (s, 1H), |
| | | | yl)methyl]quinoxalin- | [M + H]⁺ | 7.60 (d, J = 8.2 Hz, 1H), 7.41 (d, |
| | | | 6-amine | | J = 2.6 Hz, 1H), 7.39 (d, J = 3.0 Hz, |
| | | | | | 1H), 7.26 (dd, J = 8.1, 1.5 Hz, |
| | | | | | 1H), 6.82 (d, J = 2.6 Hz, 1H), |
| | | | | | 6.74 (t, J = 5.7 Hz, 1H), |
| | | | | | 6.47 (dd, J = 3.1, 0.8 Hz, 1H), |
| | | | | | 3.88-3.78 (m, 4H), 3.72 (dd, J = 11.2, |
| | | | | | 3.5 Hz, 1H), 3.54 (td, J = 11.1, |
| | | | | | 2.3 Hz, 1H), 3.31-3.21 (m, |
| | | | | | 2H), 2.85 (d, J = 11.0 Hz, 1H), |
| | | | | | 2.61 (d, J = 10.9 Hz, 1H), |
| | | | | | 2.20 (s, 3H), 2.01 (td, J = 11.3, 3.2 Hz, |
| | | | | | 1H), 1.89-1.79 (m, 1H). |
| 126 | 77 | 443.5 | 8-(1-methyl-1H- | HPLC | 1H NMR (400 MHz, DMSO): δ |
| | | | indol-6-yl)-N-{[4- | 93.6%, | 10.38 (s, 1H), 8.83 (d, J = 1.8 Hz, |
| | | | (pyrimidin-5- | m/z = 444.1 | 1H), 8.74 (d, J = 1.8 Hz, 1H), |
| | | | yl)pyridin-3- | [M + H]⁺ | 8.36 (s, 1H), 8.17 (d, J = 1.9 Hz, |
| | | | yl]methyl}quinoxalin- | | 1H), 7.84 (s, 1H), 7.82 (d, J = 5.7 Hz, |
| | | | 6-amine | | 1H), 7.80 (s, 1H), |
| | | | | | 7.67 (d, J = 2.6 Hz, 1H), 7.62 (d, J = 8.2 Hz, |
| | | | | | 1H), 7.40 (d, J = 3.1 Hz, |
| | | | | | 1H), 7.37 (ddd, J = 8.8, 4.3, 1.7 Hz, |
| | | | | | 2H), 6.48 (dd, J = 3.0, 0.8 Hz, |
| | | | | | 1H), 3.84 (s, 3H), 3.22 (s, |
| | | | | | 3H), 2.08 (s, 3H). |
| 127 | 78 | 463.6 | 8-(1-methyl-1H- | HPLC | 1H NMR (400 MHz, DMSO) δ |
| | | | indol-6-yl)-N-{[4-(4- | 96.0%, | 8.61 (d, J = 1.9 Hz, 1H), 8.48 (s, |
| | | | methylpiperazin-1- | m/z = 464.3 | 1H), 8.45 (d, J = 1.9 Hz, 1H), |
| | | | yl)pyridin-3- | [M + H]⁺ | 8.30 (d, J = 5.5 Hz, 1H), 7.61 (s, |
| | | | yl]methyl}quinoxalin- | | 1H), 7.60 (d, J = 8.2 Hz, 1H), |
| | | | 6-amine | | 7.40 (d, J = 2.6 Hz, 1H), 7.38 (d, |
| | | | | | J = 3.1 Hz, 1H), 7.32 (t, J = 5.8 Hz, |
| | | | | | 1H), 7.23 (dd, J = 8.1, 1.5 Hz, |
| | | | | | 1H), 7.01 (d, J = 5.5 Hz, 1H), |
| | | | | | 6.74 (d, J = 2.5 Hz, 1H), |
| | | | | | 6.47 (dd, J = 3.1, 0.8 Hz, 1H), |
| | | | | | 4.43 (d, J = 5.6 Hz, 2H), 3.81 (s, 3H), |
| | | | | | 3.06 (s, 4H), 2.55 (s, 4H), |
| | | | | | 2.26 (s, 3H). |
| 128 | 51 | 404.5 | N-{imidazo[1,2- | HPLC | 1H NMR (400 MHz, DMSO) δ |
| | | | a]pyridin-6- | 97.4%, | 8.62 (s, 1H), 8.61 (d, J = 1.9 Hz, |
| | | | ylmethyl}-8-(1- | m/z = 405.3 | 1H), 8.46 (d, J = 1.9 Hz, 1H), |
| | | | methyl- | [M + H]⁺ | 7.96 (s, 1H), 7.61 (s, 1H), |
| | | | 1H-indol-6- | | 7.58 (d, J = 8.4 Hz, 2H), 7.55 (d, J = 1.2 Hz, |
| | | | yl)quinoxalin-6- | | 1H), 7.44 (d, J = 2.6 Hz, |
| | | | amine | | 1H), 7.39 (d, J = 3.1 Hz, 1H), |
| | | | | | 7.32 (dd, J = 9.3, 1.7 Hz, 1H), |
| | | | | | 7.29 (s, 1H), 7.26 (dd, J = 8.2, |
| | | | | | 1.4 Hz, 1H), 6.87 (d, J = 2.6 Hz, |
| | | | | | 1H), 6.49-6.45 (m, 1H), |
| | | | | | 4.51 (d, J = 5.7 Hz, 2H), 3.81 (s, 3H). |
| 129 | 79 | 445.5 | 8-(1-methyl-1H- | HPLC | 1H NMR (400 MHz, DMSO) δ |
| | | | indol-6-yl)-N-{[4-(1- | 94.0%, | 8.62 (d, J = 1.9 Hz, 1H), 8.48 (d, |
| | | | methyl-1H- | m/z = 446.2 | J = 1.9 Hz, 1H), 8.24 (s, 1H), |
| | | | pyrazol-4-yl)pyridin- | [M + H]⁺ | 7.90 (s, 1H), 7.64 (s, 1H), |
| | | | 3- | | 7.60 (d, J = 8.2 Hz, 1H), 7.56 (d, J = 6.3 Hz, |
| | | | yl]methyl}quinoxalin- | | 1H), 7.53 (s, 1H), |
| | | | 6- | | 7.50 (dd, J = 7.2, 3.2 Hz, 1H), |
| | | | amine | | 7.46 (d, J = 2.5 Hz, 1H), 7.39 (d, J = 3.0 Hz, |
| | | | | | 1H), 7.28 (d, J = 1.4 Hz, |
| | | | | | 1H), 7.26 (q, J = 4.5 Hz, 1H), |
| | | | | | 6.72 (d, J = 2.5 Hz, 1H), 6.47 (d, |
| | | | | | J = 2.5 Hz, 1H), 4.57 (d, J = 4.8 Hz, |
| | | | | | 2H), 3.90 (s, 3H), 3.81 (s, |
| | | | | | 3H). |
| 130 | 49 | 415.5 | 1-[2-({[8-(1-methyl- | HPLC | 1H NMR (400 MHz, DMSO) δ |
| | | | 1H-indol-6- | 94.8%, | 8.66-8.59 (m, 1H), 8.45 (d, J = 1.5 Hz, |
| | | | yl)quinoxalin-6- | m/z = 416.2 | 1H), 7.62 (s, 1H), |
| | | | yl]amino}methyl)morpholin- | [M + H]⁺ | 7.59 (d, J = 8.2 Hz, 1H), 7.42 (dd, J = 9.7, |
| | | | 4-yl]ethan-1- | | 2.4 Hz, 1H), 7.37 (d, J = 3.0 Hz, |
| | | | one | | 1H), 7.26 (d, J = 8.2 Hz, 1H), |
| | | | | | 6.87 (dd, J = 19.6, 2.4 Hz, 1H), |
| | | | | | 6.75 (dt, J = 10.8, 5.6 Hz, 1H), |
| | | | | | 6.46 (d, J = 2.6 Hz, 1H), |
| | | | | | 4.40 (ddd, J = 14.6, 12.1, 5.1 Hz, |
| | | | | | 1H), 4.16 (d, J = 13.2 Hz, 1H), |
| | | | | | 3.98-3.86 (m, 2H), 3.81 (s, |
| | | | | | 3H), 3.68 (d, J = 12.2 Hz, 1H), |
| | | | | | 3.49 (td, J = 11.9, 2.9 Hz, 1H), |
| | | | | | 3.22-3.10 (m, 1H), |
| | | | | | 3.08-2.91 (m, 1H), 2.80-2.62 (m, 1H), |
| | | | | | 2.00 (s, 3H). |
| 131 | 118 | 373.5 | 8-(1-methyl-1H- | HPLC | 1H NMR (400 MHz, DMSO) δ |
| | | | indol-6-yl)-N- | 94.2%, | 8.64 (d, J = 1.9 Hz, 1H), 8.45 (d, |
| | | | (morpholin-3- | m/z = 374.3 | J = 1.9 Hz, 1H), 7.62 (s, 1H), |
| | | | ylmethyl)quinoxalin- | [M + H]⁺ | 7.60 (d, J = 8.2 Hz, 1H), 7.39 (d, |
| | | | 6-amine | | J = 3.1 Hz, 1H), 7.37 (d, J = 2.6 Hz, |
| | | | | | 1H), 7.26 (dd, J = 8.1, 1.5 Hz, |
| | | | | | 1H), 6.84 (d, J = 2.6 Hz, 1H), |
| | | | | | 6.67 (t, J = 5.6 Hz, 1H), |
| | | | | | 6.47 (dd, J = 3.1, 0.8 Hz, 1H), |
| | | | | | 3.86 (dd, J = 10.9, 2.9 Hz, 1H), |
| | | | | | 3.82 (s, 3H), 3.67 (d, J = 10.8 Hz, |
| | | | | | 1H), 3.44-3.36 (m, 1H), |
| | | | | | 3.24-3.17 (m, 1H), 3.15 (t, J = 5.8 Hz, |
| | | | | | 2H), 2.85-2.74 (m, 3H), |
| | | | | | 2.70 (d, J = 24.6 Hz, 1H). |
| 132 | 119 | 385.5 | 1-methyl-4-{[8-(1- | HPLC | 1H NMR (400 MHz, DMSO) δ |
| | | | methyl-1H-indol-6- | 93.0%, | 8.64 (d, J = 1.9 Hz, 1H), 8.46 (d, |
| | | | yl)quinoxalin-6- | m/z = 386.3 | J = 1.9 Hz, 1H), 7.69-7.50 (m, |
| | | | yl]amino}piperidin-2- | [M + H]⁺ | 2H), 7.38 (dd, J = 2.8, 1.3 Hz, |
| | | | one | | 2H), 7.27 (dd, J = 8.2, 1.5 Hz, |
| | | | | | 1H), 6.89 (d, J = 2.5 Hz, 1H), |
| | | | | | 6.73 (d, J = 7.4 Hz, 1H), |
| | | | | | 6.47 (dd, J = 3.1, 0.8 Hz, 1H), |
| | | | | | 4.01 (s, 1H), 3.81 (s, 3H), 3.39 (s, |
| | | | | | 2H), 2.85 (s, 3H), 2.69 (dd, J = 16.6, |
| | | | | | 4.1 Hz, 1H), 2.28 (dd, J = 16.9, |
| | | | | | 8.2 Hz, 1H), 2.19 (d, J = 8.6 Hz, |
| | | | | | 1H), 1.88-1.73 (m, |
| | | | | | 1H). |
| 133 | 120 | 385.5 | 1-methyl-5-{[8-(1- | HPLC | 1H NMR (400 MHz, DMSO) δ |
| | | | methyl-1H-indol-6- | 99.0%, | 8.65 (d, J = 1.9 Hz, 1H), 8.48 (d, |
| | | | yl)quinoxalin-6- | m/z = 386.3 | J = 1.9 Hz, 1H), 7.64 (s, 1H), |
| | | | yl]amino}piperidin-2- | [M + H]⁺ | 7.60 (d, J = 8.2 Hz, 1H), 7.43 (d, |
| | | | one | | J = 2.6 Hz, 1H), 7.39 (d, J = 3.1 Hz, |
| | | | | | 1H), 7.28 (dd, J = 8.2, 1.4 Hz, |
| | | | | | 1H), 6.97 (d, J = 2.5 Hz, 1H), |
| | | | | | 6.78 (d, J = 7.7 Hz, 1H), |
| | | | | | 6.47 (dd, J = 3.0, 0.7 Hz, 1H), |
| | | | | | 4.07 (s, 1H), 3.82 (s, 3H), 3.64 (dd, J = 12.1, |
| | | | | | 4.4 Hz, 1H), 3.25 (dd, J = 12.1, |
| | | | | | 6.9 Hz, 1H), 2.84 (s, |
| | | | | | 3H), 2.41 (t, J = 6.7 Hz, 2H), |
| | | | | | 2.17-2.05 (m, 1H), 1.88 (td, J = 15.4, |
| | | | | | 7.4 Hz, 1H). |
| 134 | 121 | 368.5 | N-[(1-methyl-1H- | HPLC | 1H NMR (400 MHz, DMSO) δ |
| | | | imidazol-5- | 96.4% | 8.64 (d, J = 1.9 Hz, 1H), 8.47 (d, |
| | | | yl)methyl]-8-(1- | m/z = 369.5 | J = 1.9 Hz, 1H), 7.61 (d, J = 1.8 Hz, |
| | | | methyl- | | 2H), 7.59 (d, J = 3.2 Hz, 1H), |
| | | | 1H-indol-6- | | 7.42 (d, J = 2.6 Hz, 1H), 7.38 (d, |
| | | | yl)quinoxalin-6- | | J = 3.1 Hz, 1H), 7.25 (dd, J = 8.1, |
| | | | amine | | 1.5 Hz, 1H), |
| | | | | | 7.00-6.94 (m, 3H), 6.47 (dd, J = 3.1, 0.8 Hz, |
| | | | | | 1H), 4.44 (d, J = 5.2 Hz, 2H), |
| | | | | | 3.81 (s, 3H), 3.66 (s, 3H). |
| 135 | Intermediate | 444.3 | N-[(4-bromopyridin- | HPLC | 1H NMR (400 MHz, DMSO) δ |
| | 73 | | 2-yl)methyl]-8-(1- | 95.2%; | 8.62 (d, J = 1.9 Hz, 1H), |
| | | | methyl-1H-indol-6- | m/z | 8.50-8.46 (m, 2H), 7.72 (d, J = 1.6 Hz, |
| | | | yl)quinoxalin-6- | 444.0 | 1H), 7.63 (s, 1H), |
| | | | amine | [M + H]+ | 7.62-7.58 (m, 2H), 7.47 (d, J = 2.6 Hz, |
| | | | | | 1H), 7.41-7.36 (m, 2H), |
| | | | | | 7.27 (dd, J = 8.2, 1.5 Hz, 1H), |
| | | | | | 6.74 (d, J = 2.6 Hz, 1H), |
| | | | | | 6.48 (dd, J = 3.0, 0.8 Hz, 1H), |
| | | | | | 4.59 (d, J = 6.0 Hz, 2H), 3.82 (s, 3H). |
| 136 | 122 | 445.5 | 8-(1-methyl-1H- | HPLC | 1H NMR (400 MHz, DMSO) δ |
| | | | indol-6-yl)-N-{[4-(1- | 94.4%; | 8.60 (d, J = 1.9 Hz, 1H), 8.50 (d, |
| | | | methyl-1H- | m/z = 446.2 | J = 5.2 Hz, 1H), 8.45 (d, J = 1.9 Hz, |
| | | | pyrazol-4-yl)pyridin- | [M + H]+ | 1H), 8.32 (s, 1H), 8.00 (d, J = 0.6 Hz, |
| | | | 2- | | 1H), 7.67-7.65 (m, |
| | | | yl]methyl}quinoxalin- | | 1H), 7.62-7.58 (m, 2H), |
| | | | 6- | | 7.50 (d, J = 2.6 Hz, 1H), 7.48 (dd, J = 5.2, |
| | | | amine | | 1.6 Hz, 1H), 7.38 (d, J = 3.1 Hz, |
| | | | | | 1H), 7.31 (t, J = 5.9 Hz, 1H), |
| | | | | | 7.26 (dd, J = 8.1, 1.5 Hz, 1H), |
| | | | | | 6.80 (d, J = 2.6 Hz, 1H), |
| | | | | | 6.47 (dd, J = 3.1, 0.7 Hz, 1H), |
| | | | | | 4.54 (d, J = 5.7 Hz, 2H), 3.86 (s, 3H), |
| | | | | | 3.81 (s, 3H). |
| 137 | Intermediate | 444.3 | N-[(2-bromopyridin- | HPLC | 1H NMR (400 MHz, DMSO) δ |
| | 74 | | 4-yl)methyl]-8-(1- | 92.3%; | 8.62 (d, J = 1.9 Hz, 1H), 8.48 (d, |
| | | | methyl-1H- | m/z = 444.1 | J = 1.9 Hz, 1H), 8.36 (d, J = 5.1 Hz, |
| | | | indol-6-yl)quinoxalin- | [M + H]+ | 1H), 7.71 (s, 1H), 7.63 (s, |
| | | | 6-amine | | 1H), 7.61 (d, J = 8.2 Hz, 1H), |
| | | | | | 7.50 (dd, J = 5.1, 1.3 Hz, 1H), |
| | | | | | 7.44 (d, J = 2.6 Hz, 1H), |
| | | | | | 7.41-7.37 (m, 2H), 7.27 (dd, J = 8.2, |
| | | | | | 1.4 Hz, 1H), 6.72 (d, J = 2.6 Hz, |
| | | | | | 1H), 6.48 (dd, J = 3.1, 0.7 Hz, |
| | | | | | 1H), 4.58 (d, J = 6.1 Hz, 2H), |
| | | | | | 3.82 (s, 3H). |
| 138 | 123 | 445.5 | 8-(1-methyl-1H- | HPLC | 1H NMR (400 MHz, DMSO) δ |
| | | | indol-6-yl)-N-{[2-(1- | 96.6%, | 8.60 (d, J = 1.9 Hz, 1H), |
| | | | methyl-1H- | m/z = 446.3 | 8.47-8.44 (m, 2H), 8.25 (s, 1H), |
| | | | pyrazol-4-yl)pyridin- | [M + H]+ | 7.97 (d, J = 0.5 Hz, 1H), 7.72 (s, 1H), |
| | | | 4- | | 7.63-7.58 (m, 2H), 7.46 (d, J = 2.6 Hz, |
| | | | yl]methyl}quinoxalin- | | 1H), 7.38 (d, J = 3.1 Hz, |
| | | | 6- | | 1H), 7.37-7.34 (m, 1H), |
| | | | amine | | 7.27 (dd, J = 8.1, 1.4 Hz, 1H), |
| | | | | | 7.23 (dd, J = 5.1, 1.4 Hz, 1H), |
| | | | | | 6.74 (d, J = 2.6 Hz, 1H), 6.47 (dd, J = 3.0, |
| | | | | | 0.7 Hz, 1H), 4.53 (d, J = 5.9 Hz, |
| | | | | | 2H), 3.88 (s, 3H), 3.80 (s, |
| | | | | | 3H). |
| 139 | 124 | 446.5 | N-[(1-methyl-1H- | HPLC | 1H NMR (400 MHz, DMSO) δ |
| | | | 1,2,3-triazol-5- | 92%; | 8.81 (d, J = 1.6 Hz, 1H), 8.65 (d, |
| | | | yl)(pyridin-3- | m/z = 447.3 | J = 1.9 Hz, 1H), 8.57 (dd, J = 4.6, |
| | | | yl)methyl]-8-(1- | [M + H]+ | 1.3 Hz, 1H), 8.51 (d, J = 1.9 Hz, |
| | | | methyl-1H-indol-6- | | 1H), 8.00-7.87 (m, 1H), |
| | | | yl)quinoxalin-6- | | 7.65-7.57 (m, J = 10.9, 7.9 Hz, |
| | | | amine | | 3H), 7.52 (d, J = 2.6 Hz, 1H), |
| | | | | | 7.48 (dd, J = 7.7, 4.6 Hz, 1H), |
| | | | | | 7.39 (d, J = 3.1 Hz, 1H), 7.35 (s, |
| | | | | | 1H), 7.26 (dd, J = 8.2, 1.4 Hz, |
| | | | | | 1H), 6.97 (d, J = 2.5 Hz, 1H), |
| | | | | | 6.47 (d, J = 3.0 Hz, 1H), 6.43 (d, |
| | | | | | J = 7.8 Hz, 1H), 4.06 (s, 3H), |
| | | | | | 3.81 (s, 3H). |
| 140 | 125 | 462.6 | 8-(1-methyl-1H- | HPLC | 1H NMR (400 MHz, DMSO) δ |
| | | | indol-6-yl)-N-[(1- | 96.6%; | 8.71 (d, J = 1.8 Hz, 1H), 8.56 (d, |
| | | | methylpiperidin-4- | m/z = 463.5 | J = 1.9 Hz, 1H), 8.43 (dd, J = 4.7, |
| | | | yl)(pyridin-3- | [M + H]+ | 1.6 Hz, 1H), 8.41 (d, J = 1.9 Hz, |
| | | | yl)methyl]quinoxalin- | | 1H), 7.85 (dt, J = 7.8, 1.9 Hz, |
| | | | 6-amine | | 1H), 7.61-7.57 (m, 2H), |
| | | | | | 7.50 (d, J = 2.6 Hz, 1H), 7.38 (d, |
| | | | | | J = 3.0 Hz, 1H), 7.38-7.34 (m, |
| | | | | | 1H), 7.26 (d, J = 5.3 Hz, 1H), |
| | | | | | 7.24 (d, J = 1.4 Hz, 1H), 6.69 (d, |
| | | | | | J = 2.4 Hz, 1H), 6.47 (dd, J = 3.0, |
| | | | | | 0.7 Hz, 1H), 4.47 (t, J = 8.0 Hz, |
| | | | | | 1H), 3.81 (s, 3H), 2.78 (dd, J = 39.6, |
| | | | | | 11.1 Hz, 2H), 2.13 (s, |
| | | | | | 3H), 1.89-1.59 (m, 4H), |
| | | | | | 1.50-1.24 (m, 3H) |
| 141 | 126 | 463.6 | N-[(4- | HPLC | 1H NMR (400 MHz, DMSO) δ |
| | | | benzylmorpholin-3- | 96.9%; | 8.64 (d, J = 1.9 Hz, 1H), 8.45 (d, |
| | | | yl)methyl]-8-(1- | m/z = 464.3 | J = 1.9 Hz, 1H), 7.63 (s, 1H), |
| | | | methyl-1H- | [M + H] | 7.60 (d, J = 8.2 Hz, 1H), 7.44 (d, |
| | | | indol-6-yl)quinoxalin- | | J = 2.5 Hz, 1H), 7.41 (s, 1H), |
| | | | 6-amine | | 7.39 (d, J = 2.8 Hz, 2H), 7.32 (t, |
| | | | | | J = 7.5 Hz, 2H), 7.27 (dd, J = 8.2, |
| | | | | | 1.3 Hz, 1H), 7.23 (t, J = 7.3 Hz, |
| | | | | | 1H), 6.84 (d, J = 2.5 Hz, 1H), |
| | | | | | 6.60 (s, 1H), 6.48 (d, J = 2.6 Hz, |
| | | | | | 1H), 4.13 (d, J = 13.6 Hz, 1H), |
| | | | | | 3.82 (s, 3H), 3.64-3.51 (m, |
| | | | | | 4H), 3.43 (d, J = 13.5 Hz, 2H), |
| | | | | | 2.75 (s, 2H), 2.67 (s, 2H). |
| 142 | 127 | 451.5 | 8-(1-methyl-1H- | HPLC | 1H NMR (400 MHz, DMSO) δ |
| | | | indol-6-yl)-N-{[4- | 92.0%; | 8.64 (d, J = 1.9 Hz, 1H), 8.62 (s, |
| | | | (pyrimidin-5- | m/z = 452.4 | 1H), 8.53 (s, 2H), 8.46 (d, J = 1.9 Hz, |
| | | | yl)morpholin-2- | [M + H] | 1H), 7.66-7.55 (m, |
| | | | yl]methyl}quinoxalin- | | 2H), 7.45 (d, J = 2.6 Hz, 1H), |
| | | | 6-amine | | 7.38 (d, J = 3.1 Hz, 1H), |
| | | | | | 7.27 (dd, J = 8.2, 1.4 Hz, 1H), |
| | | | | | 6.91 (d, J = 2.6 Hz, 1H), 6.79 (t, J = 5.8 Hz, |
| | | | | | 1H), 6.47 (d, J = 3.9 Hz, |
| | | | | | 1H), 3.82 (s, 3H), 3.70 (t, J = 10.3 Hz, |
| | | | | | 2H), 3.52-3.33 (m, |
| | | | | | 3H), 2.86 (dd, J = 12.3, 9.0 Hz, |
| | | | | | 1H), 2.75-2.65 (m, 2H). |
| 143 | 128 | 389.46 | 3-({[8-(1-methyl-1H- | HPLC | 1H NMR (400 MHz, DMSO) δ |
| | | | indol-5-yl)quinoxalin- | 95.0%; | 8.60 (d, J = 1.9 Hz, 1H), 8.46 (d, |
| | | | 6- | m/z = 390.3 | J = 1.9 Hz, 1H), 7.91 (s, 1H), |
| | | | yl]amino}methyl)benzonitrile | [M + H]⁺ | 7.80 (d, J = 7.8 Hz, 1H), 7.75 (d, |
| | | | | | J = 8.4 Hz, 2H), 7.59 (t, J = 7.7 Hz, |
| | | | | | 1H), 7.51 (d, J = 8.6 Hz, 1H), |
| | | | | | 7.42 (d, J = 2.6 Hz, 1H), |
| | | | | | 7.39 (dd, J = 9.6, 2.4 Hz, 2H), 7.33 (t, |
| | | | | | J = 6.0 Hz, 1H), 6.73 (d, J = 2.6 Hz, |
| | | | | | 1H), 6.49 (d, J = 3.0 Hz, 1H), |
| | | | | | 4.56 (d, J = 5.9 Hz, 2H), 3.84 (s, |
| | | | | | J = 8.5 Hz, 3H). |
| 144 | 129 | 407.48 | 3-({[8-(1-methyl-1H- | HPLC | 1H NMR (400 MHz, DMSO) δ |
| | | | indol-5-yl)quinoxalin- | 98.2%; | 8.59 (d, J = 1.9 Hz, 1H), 8.44 (d, |
| | | | 6- | m/z = 408.4 | J = 1.9 Hz, 1H), 7.99 (s, 2H), |
| | | | yl]amino}methyl)benzamide | [M + H]+ | 7.77 (t, J = 5.2 Hz, 2H), 7.60 (d, |
| | | | | | J = 7.5 Hz, 1H), 7.50 (d, J = 8.5 Hz, |
| | | | | | 1H), 7.42 (td, J = 11.3, 4.6 Hz, |
| | | | | | 3H), 7.37 (d, J = 3.1 Hz, 2H), |
| | | | | | 7.31 (t, J = 5.8 Hz, 1H), 6.73 (d, |
| | | | | | J = 2.6 Hz, 1H), 6.49 (d, J = 3.0 Hz, |
| | | | | | 1H), 4.52 (d, J = 5.8 Hz, 2H), |
| | | | | | 3.84 (s, 3H). |
| 145 | 130 | 393.49 | N-[4-(1- | HPLC | 1H NMR (400 MHz, DMSO) δ |
| | | | aminoethyl)phenyl]- | 96.6%; | 8.85 (s, 1H), 8.70 (d, J = 1.8 Hz, |
| | | | 8-(1-methyl-1H- | m/z = 394.3 | 1H), 8.56 (d, J = 1.9 Hz, 1H), |
| | | | indol-5-yl)quinoxalin- | [M + H]+ | 7.81 (d, J = 1.0 Hz, 1H), 7.57 (d, |
| | | | 6-amine | | J = 2.6 Hz, 1H), 7.53 (d, J = 8.5 Hz, |
| | | | | | 1H), 7.43 (dd, J = 8.5, 1.5 Hz, |
| | | | | | 1H), 7.38 (dd, J = 6.9, 3.8 Hz, |
| | | | | | 4H), 7.27 (d, J = 8.4 Hz, 2H), |
| | | | | | 6.50 (d, J = 3.0 Hz, 1H), 3.98 (q, |
| | | | | | J = 6.5 Hz, 1H), 3.85 (s, 3H), |
| | | | | | 1.83 (s, 2H), 1.26 (d, J = 6.6 Hz, |
| | | | | | 3H). |
| 146 | 131 | 399.50 | 1-(4-{[8-(1-methyl- | HPLC | 1H NMR (400 MHz, DMSO) δ |
| | | | 1H-indol-5- | 96.2%; | 8.62 (d, J = 1.9 Hz, 1H), 8.44 (d, |
| | | | yl)quinoxalin-6- | m/z = 400.4 | J = 1.9 Hz, 1H), 7.76 (d, J = 1.0 Hz, |
| | | | yl]amino}piperidin-1- | [M + H]⁺ | 1H), 7.50 (d, J = 8.5 Hz, 1H), |
| | | | yl)ethan-1-one | | 7.37 (td, J = 10.0, 2.0 Hz, 3H), |
| | | | | | 6.88 (d, J = 2.5 Hz, 1H), 6.59 (d, |
| | | | | | J = 7.7 Hz, 1H), 6.50-6.46 (m, |
| | | | | | 1H), 4.31-4.20 (m, 1H), |
| | | | | | 3.88-3.70 (m, 5H), 3.31-3.20 (m, |
| | | | | | 1H), 2.97-2.84 (m, 1H), |
| | | | | | 2.12-1.95 (m, 5H), 1.47-1.28 (m, |
| | | | | | 2H). |
| 147 | 132 | 413.53 | 1-(4-{[8-(1-ethyl-1H- | HPLC | 1H NMR (400 MHz, DMSO) δ |
| | | | indol-6-yl)quinoxalin- | 95.1%; | 8.63 (d, J = 1.9 Hz, 1H), 8.44 (d, |
| | | | 6-yl]amino}piperidin- | m/z = 414.4 | J = 1.9 Hz, 1H), 7.65 (s, 1H), |
| | | | 1-yl)ethan-1-one | [M + H]⁺ | 7.59 (d, J = 8.2 Hz, 1H), 7.45 (d, |
| | | | | | J = 3.1 Hz, 1H), 7.37 (d, J = 2.6 Hz, |
| | | | | | 1H), 7.26 (dd, J = 8.2, 1.4 Hz, |
| | | | | | 1H), 6.90 (d, J = 2.5 Hz, 1H), |
| | | | | | 6.62 (d, J = 7.7 Hz, 1H), 6.48 (d, |
| | | | | | J = 3.1 Hz, 1H), 4.34-4.18 (m, |
| | | | | | 3H), 3.84 (d, J = 14.2 Hz, 1H), |
| | | | | | 3.74 (s, 1H), 3.27 (t, J = 7.1 Hz, |
| | | | | | 1H), 2.90 (t, J = 11.0 Hz, 1H), |
| | | | | | 2.13-1.95 (m, 5H), |
| | | | | | 1.46-1.27 (m, 5H). |
| 148 | 133 | 413.53 | 1-(4-{[8-(1-ethyl-1H- | HPLC | 1H NMR (400 MHz, DMSO) δ |
| | | | indol-5-yl)quinoxalin- | 99.5%; | 8.62 (d, J = 1.9 Hz, 1H), 8.44 (d, |
| | | | 6-yl]amino}piperidin- | m/z = 414.5 | J = 1.9 Hz, 1H), 7.75 (d, J = 1.1 Hz, |
| | | | 1-yl)ethan-1-one | [M + H]⁺ | 1H), 7.54 (d, J = 8.6 Hz, 1H), |
| | | | | | 7.44 (d, J = 3.1 Hz, 1H), |
| | | | | | 7.37 (dd, J = 8.5, 1.6 Hz, 1H), |
| | | | | | 7.34 (d, J = 2.6 Hz, 1H), 6.88 (d, J = 2.5 Hz, |
| | | | | | 1H), 6.59 (d, J = 7.8 Hz, |
| | | | | | 1H), 6.49 (d, J = 2.6 Hz, 1H), |
| | | | | | 4.26 (q, J = 7.1 Hz, 3H), 3.83 (d, |
| | | | | | J = 14.1 Hz, 1H), |
| | | | | | 3.78-3.67 (m, 1H), 3.31-3.22 (m, 1H), |
| | | | | | 2.90 (t, J = 10.9 Hz, 1H), |
| | | | | | 2.13-1.96 (m, 5H), 1.40 (t, J = 7.2 Hz, |
| | | | | | 4H), 1.36-1.21 (m, 1H). |
| 149 | 134 | 475.60 | 1-(4-{[8-(1-benzyl- | HPLC | 1H NMR (400 MHz, DMSO) δ |
| | | | 1H-indol-5- | 97.9%; | 8.61 (d, J = 1.9 Hz, 1H), 8.43 (d, |
| | | | yl)quinoxalin-6- | m/z = 476.3 | J = 1.9 Hz, 1H), 7.76 (d, J = 1.1 Hz, |
| | | | yl]amino}piperidin-1- | [M + H]⁺ | 1H), 7.57 (d, J = 3.1 Hz, 1H), |
| | | | yl)ethan-1-one | | 7.50 (d, J = 8.6 Hz, 1H), |
| | | | | | 7.38-7.29 (m, 4H), 7.29-7.19 (m, |
| | | | | | 3H), 6.87 (d, J = 2.5 Hz, 1H), |
| | | | | | 6.62-6.50 (m, 2H), 5.47 (s, |
| | | | | | 2H), 4.30-4.20 (m, 1H), |
| | | | | | 3.87-3.78 (m, 1H), 3.78-3.67 (m, |
| | | | | | 1H), 3.31-3.22 (m, 1H), |
| | | | | | 2.95-2.84 (m, 1H), 2.10-1.94 (m, |
| | | | | | 5H), 1.46-1.35 (m, 1H), |
| | | | | | 1.34-1.24 (m, 1H). |
| 153 | 138 | 385.47 | 1-[(3S)-3-{[8-(1- | HPLC | 1H NMR (400 MHz, DMSO) δ |
| | | | methyl-1H-indol-6- | 100%; | 8.66 (d, J = 1.2 Hz, 1H), |
| | | | yl)quinoxalin-6- | m/z = 386.3 | 8.52-8.45 (m, 1H), 7.67-7.54 (m, |
| | | | yl]amino}pyrrolidin- | [M + H]⁺ | 2H), 7.38 (t, J = 3.4 Hz, 2H), |
| | | | 1-yl]ethan-1-one | | 7.28 (ddd, J = 8.2, 2.8, 1.4 Hz, |
| | | | | | 1H), 6.89 (ddd, J = 13.0, 8.6, |
| | | | | | 4.6 Hz, 2H), 6.48 (d, J = 3.0 Hz, |
| | | | | | 1H), 4.32-4.17 (m, 1H), |
| | | | | | 4.11-3.98 (m, 1H), 3.82 (s, 3H), |
| | | | | | 3.63 (ddd, J = 12.3, 10.2, 5.3 Hz, |
| | | | | | 2H), 3.44-3.36 (m, 1H), |
| | | | | | 2.41-2.11 (m, 2H), 2.08-1.89 (m, |
| | | | | | 5H), 1.18 (dd, J = 9.1, 5.2 Hz, |
| | | | | | 1H). |
| 154 | 139 | 385.47 | 1-[(3R)-3-{[8-(1- | HPLC | 1H NMR (400 MHz, DMSO) δ |
| | | | methyl-1H-indol-6- | 99.0%; | 8.66 (d, J = 1.3 Hz, 1H), 8.48 (t, |
| | | | yl)quinoxalin-6- | m/z = 386.2 | J = 1.6 Hz, 1H), 7.66-7.55 (m, |
| | | | yl]amino}pyrrolidin- | [M + H]⁺ | 2H), 7.38 (t, J = 3.6 Hz, 2H), |
| | | | 1-yl]ethan-1-one | | 7.27 (ddd, J = 8.2, 2.9, 1.4 Hz, |
| | | | | | 1H), 6.96-6.83 (m, 2H), |
| | | | | | 6.47 (d, J = 3.0 Hz, 1H), |
| | | | | | 4.32-4.16 (m, 1H), 3.88 (dd, J = 10.4, 5.8 Hz, |
| | | | | | 0.5H), 3.81 (s, 3H), |
| | | | | | 3.68-3.57 (m, 1.5H), 3.50-3.44 (m, |
| | | | | | 1H), 3.39 (dd, J = 11.1, 3.5 Hz, |
| | | | | | 1H), 2.35-2.15 (m, 1H), |
| | | | | | 2.07-1.89 (m, 4H). Mixture of two |
| | | | | | conformers in 1:1 ratio |
| 155 | 140 | 371.44 | 1-(3-{[8-(1-methyl- | HPLC | 1H NMR (400 MHz, DMSO) δ |
| | | | 1H-indol-6- | 99.9%; | 8.67 (d, J = 1.9 Hz, 1H), 8.51 (d, |
| | | | yl)quinoxalin-6- | m/z = 372.2 | J = 1.9 Hz, 1H), 7.63 (s, 1H), |
| | | | yl]amino}azetidin-1- | [M + H]⁺ | 7.61 (d, J = 8.2 Hz, 1H), 7.39 (d, |
| | | | yl)ethan-1-one | | J = 3.0 Hz, 1H), 7.34 (d, J = 2.6 Hz, |
| | | | | | 1H), 7.32-7.24 (m, 2H), |
| | | | | | 6.71 (d, J = 2.5 Hz, 1H), 6.47 (d, |
| | | | | | J = 3.0 Hz, 1H), 4.59 (t, J = 7.8 Hz, |
| | | | | | 1H), 4.45-4.35 (m, 1H), |
| | | | | | 4.33-4.25 (m, 1H), 3.97 (dd, J = 8.5, |
| | | | | | 4.7 Hz, 1H), 3.81 (s, 3H), |
| | | | | | 3.76 (dd, J = 9.6, 4.8 Hz, 1H), |
| | | | | | 1.80 (s, 3H). |
| 156 | 141 | 399.50 | 1-[(3S)-3-{[8-(1- | HPLC | 1H NMR (400 MHz, DMSO) δ |
| | | | methyl-1H-indol-6- | 94.1%; | 8.69-8.63 (m, 1H), 8.47 (dd, J = 3.4, |
| | | | yl)quinoxalin-6- | m/z = 400.3 | 1.9 Hz, 1H), 7.64 (d, J = 4.3 Hz, |
| | | | yl]amino}piperidin-1- | [M + H]⁺ | 1H), 7.60 (dd, J = 8.2, |
| | | | yl]ethan-1-one | | 2.2 Hz, 1H), 7.47 (d, J = 2.5 Hz, |
| | | | | | 1H), 7.39 (dd, J = 5.2, 2.0 Hz, |
| | | | | | 2H), 7.28 (ddd, J = 8.1, 3.8, 1.4 Hz, |
| | | | | | 1H), 6.93 (dd, J = 8.5, 2.5 Hz, |
| | | | | | 1H), 6.65 (dd, J = 17.5, 7.5 Hz, |
| | | | | | 1H), 6.48 (d, J = 3.0 Hz, 1H), |
| | | | | | 4.55 (d, J = 10.7 Hz, 1H), |
| | | | | | 3.82 (d, J = 2.3 Hz, 3H), |
| | | | | | 3.81-3.64 (m, 2H), 3.41 (dd, J = 17.6, 10.0 Hz, |
| | | | | | 1H), 3.26-3.08 (m, 3H), |
| | | | | | 2.58 (dd, J = 12.5, 9.5 Hz, 1H), |
| | | | | | 2.13-1.95 (m, 4H), |
| | | | | | 1.88-1.71 (m, 1H), 1.70-1.42 (m, |
| | | | | | 2H). Mixture of two |
| | | | | | conformers |
| 157 | 142 | 399.50 | 1-[(3R)-3-{[8-(1- | HPLC | 1H NMR (400 MHz, DMSO) δ |
| | | | methyl-1H-indol-6- | 98.1%; | 8.67-8.61 (m, 1H), 8.46 (dd, J = 3.4, |
| | | | yl)quinoxalin-6- | m/z = 400.3 | 1.9 Hz, 1H), 7.64 (d, J = 4.1 Hz, |
| | | | yl]amino}piperidin-1- | [M + H]⁺ | 1H), 7.60 (dd, J = 8.2, |
| | | | yl]ethan-1-one | | 2.2 Hz, 1H), 7.49-7.35 (m, |
| | | | | | 2H), 7.27 (ddd, J = 8.2, 3.8, 1.3 Hz, |
| | | | | | 1H), 6.92 (dd, J = 8.4, 2.4 Hz, |
| | | | | | 1H), 6.64 (dd, J = 17.4, 7.7 Hz, |
| | | | | | 1H), 6.47 (d, J = 3.0 Hz, 1H), |
| | | | | | 4.58-4.51 (m, 0.5H), |
| | | | | | 3.85-3.66 (m, 5H), 3.46-3.38 (m, |
| | | | | | 0.5H), 3.24-3.08 (m, |
| | | | | | 1H + MeOH), 2.57 (dd, J = 12.5, |
| | | | | | 9.8 Hz, 1H), 2.13-1.94 (m, |
| | | | | | 4H), 1.85-1.72 (m, 1H), |
| | | | | | 1.66-1.44 (m, 2H). |
| 158 | 143 | 343.43 | 8-(1-methyl-1H- | HPLC | 1H NMR (400 MHz, DMSO) δ |
| | | | indol-6-yl)-N- | 94.2%; | 8.62 (d, J = 1.8 Hz, 1H), 8.44 (d, |
| | | | (pyrrolidin-3- | m/z = 344.2 | J = 1.8 Hz, 1H), 7.65-7.56 (m, |
| | | | yl)quinoxalin-6- | [M + H]⁺ | 2H), 7.36 (dd, J = 13.5, 2.7 Hz, |
| | | | amine | | 2H), 7.26 (dd, J = 8.1, 1.1 Hz, |
| | | | | | 1H), 6.77 (d, J = 2.3 Hz, 1H), |
| | | | | | 6.71 (d, J = 6.3 Hz, 1H), 6.47 (d, |
| | | | | | J = 2.9 Hz, 1H), 4.02-3.92 (m, |
| | | | | | 1H), 3.81 (s, 3H), 3.11 (dd, J = 11.2, |
| | | | | | 6.2 Hz, 1H), |
| | | | | | 2.96-2.87 (m, 1H), 2.87-2.78 (m, 1H), |
| | | | | | 2.73 (dd, J = 11.3, 3.7 Hz, 1H), |
| | | | | | 2.09 (dt, J = 20.2, 7.5 Hz, 1H), |
| | | | | | 1.74-1.61 (m, 1H). |
| 159 | 144 | 420.52 | 8-(1-methyl-1H- | HPLC | 1H NMR (400 MHz, DMSO) δ |
| | | | indol-6-yl)-N-[(3S)-1- | 99.3%; | 8.66 (d, J = 1.9 Hz, 1H), 8.47 (d, |
| | | | (pyridin-2- | m/z = 421.2 | J = 1.9 Hz, 1H), 8.06 (dd, J = 4.9, |
| | | | yl)pyrrolidin-3- | [M + H]⁺ | 1.2 Hz, 1H), 7.63 (s, 1H), |
| | | | yl]quinoxalin-6- | | 7.59 (d, J = 8.2 Hz, 1H), |
| | | | amine | | 7.48 (ddd, J = 8.8, 7.1, 1.9 Hz, 1H), |
| | | | | | 7.39 (d, J = 2.6 Hz, 1H), 7.37 (d, |
| | | | | | J = 3.1 Hz, 1H), 7.27 (dd, J = 8.2, |
| | | | | | 1.4 Hz, 1H), 6.94 (d, J = 6.4 Hz, |
| | | | | | 1H), 6.91 (d, J = 2.5 Hz, 1H), |
| | | | | | 6.54 (dd, J = 6.7, 5.2 Hz, 1H), |
| | | | | | 6.52-6.44 (m, 2H), |
| | | | | | 4.39-4.30 (m, 1H), 3.87-3.75 (m, 4H), |
| | | | | | 3.65-3.45 (m, 3H), |
| | | | | | 2.44-2.34 (m, 1H), 2.15-2.04 (m, 1H). |
| 160 | 145 | 448.53 | 8-(1-methyl-1H- | HPLC | Not determined |
| | | | indol-6-yl)-N-[(3S)-1- | 98.7%; | |
| | | | (pyridine-2- | m/z = 449.3 | |
| | | | carbonyl)pyrrolidin- | [M + H]⁺ | |
| | | | 3-yl]quinoxalin-6- | | |
| | | | amine | | |
| 161 | 146 | 459.56 | N-[(3S)-1-(1H-1,3- | HPLC | 1H NMR (400 MHz, DMSO) ? |
| | | | benzodiazol-2- | 99.8%; | 11.20 (s, 1H), 8.67 (d, J = 1.9 Hz, |
| | | | yl)pyrrolidin-3-yl]-8- | m/z = 460.3 | 1H), 8.49 (d, J = 1.9 Hz, 1H), |
| | | | (1-methyl-1H-indol- | [M + H]⁺ | 7.63 (s, 1H), 7.59 (d, J = 8.2 Hz, |
| | | | 6-yl)quinoxalin-6- | | 1H), 7.40 (d, J = 2.5 Hz, 1H), |
| | | | amine | | 7.37 (d, J = 3.0 Hz, 1H), |
| | | | | | 7.28 (dd, J = 8.2, 1.4 Hz, 1H), |
| | | | | | 7.22-7.08 (m, 2H), 6.99 (d, J = 6.4 Hz, |
| | | | | | 1H), 6.95-6.78 (m, 3H), |
| | | | | | 6.46 (dd, J = 3.0, 0.5 Hz, 1H), |
| | | | | | 4.43-4.32 (m, 1H), 3.90 (dd, J = 10.3, |
| | | | | | 5.7 Hz, 1H), 3.80 (s, |
| | | | | | 3H), 3.76-3.60 (m, 2H), |
| | | | | | 3.57 (dd, J = 10.2, 3.1 Hz, 1H), |
| | | | | | 2.47-2.36 (m, 1H), 2.12 (ddd, J = 11.5, |
| | | | | | 7.6, 4.4 Hz, 1H). |
| 162 | 147 | 411.51 | N-(1- | HPLC | 1H NMR (400 MHz, DMSO) δ |
| | | | cyclopropanecarbonylpyrrolidin- | 97.6%; | 8.66 (s, 2H), 8.49 (s, 2H), |
| | | | 3-yl)-8- | m/z = 412.2 | 7.64 (s, 2H), 7.61 (d, J = 8.0 Hz, 2H), |
| | | | (1-methyl-1H-indol- | [M + H]⁺ | 7.40 (dd, J = 7.0, 2.5 Hz, 4H), |
| | | | 6-yl)quinoxalin-6- | | 7.28 (dd, J = 8.0, 2.2 Hz, 2H), |
| | | | amine | | 7.00-6.86 (m, 4H), 6.48 (d, J = 2.8 Hz, |
| | | | | | 2H), 4.41-4.29 (m, |
| | | | | | 1H), 4.27-4.18 (m, 1H), |
| | | | | | 4.06 (dd, J = 10.2, 5.8 Hz, 1H), |
| | | | | | 3.82 (s, 8H), 3.72-3.58 (m, 2H), |
| | | | | | 3.56-3.39 (m, 3H), 2.35 (dd, J = 13.6, |
| | | | | | 4.8 Hz, 1H), |
| | | | | | 2.29-2.17 (m, 1H), 2.13-2.02 (m, 1H), |
| | | | | | 2.01-1.88 (m, 1H), |
| | | | | | 1.87-1.73 (m, 2H), 0.79-0.68 (m, |
| | | | | | 8H). Mixture 1:1 of |
| | | | | | diastereomeric conformers |
| 163 | 148 | 421.52 | N-(1- | HPLC | 1H NMR (400 MHz, DMSO) δ |
| | | | methanesulfonylpyrrolidin- | 98.6%; | 8.67 (d, J = 1.9 Hz, 1H), 8.50 (d, |
| | | | 3-yl)-8-(1- | m/z = 422.1 | J = 1.9 Hz, 1H), 7.64 (s, 1H), |
| | | | methyl-1H-indol-6- | [M + H]⁺ | 7.61 (d, J = 8.2 Hz, 1H), 7.39 (d, |
| | | | yl)quinoxalin-6- | | J = 3.1 Hz, 2H), 7.28 (dd, J = 8.2, |
| | | | amine | | 1.4 Hz, 1H), 6.90 (t, J = 4.9 Hz, |
| | | | | | 2H), 6.48 (dd, J = 3.0, 0.7 Hz, |
| | | | | | 1H), 4.33 -4.25 (m, 1H), |
| | | | | | 3.82 (s, 3H), 3.68 (dd, J = 10.4, |
| | | | | | 5.9 Hz, 1H), 3.52-3.38 (m, |
| | | | | | 2H), 3.24 (dd, J = 10.3, 3.8 Hz, |
| | | | | | 1H), 2.94 (s, 3H), |
| | | | | | 2.40-2.29 (m, 1H), 2.03-1.96 (m, 1H). |
| 164 | 149 | 399.50 | 1-(3-{[8-(1-methyl- | HPLC | 1H NMR (400 MHz, DMSO) δ |
| | | | 1H-indol-6- | 93.0%; | 8.65 (d, J = 1.6 Hz, 2H), 8.48 (t, |
| | | | yl)quinoxalin-6- | m/z = 400.2 | J = 1.7 Hz, 2H), 7.63 (s, 2H), |
| | | | yl]amino}pyrrolidin- | [M + H]⁺ | 7.60 (d, J = 8.2 Hz, 2H), |
| | | | 1-yl)propan-1-one | | 7.43-7.32 (m, 4H), 7.27 (ddd, J = 8.2, |
| | | | | | 2.6, 1.5 Hz, 2H), 6.94-6.79 (m, |
| | | | | | 4H), 6.47 (d, J = 2.9 Hz, 2H), |
| | | | | | 4.33-4.25 (m, 1H), |
| | | | | | 4.22-4.15 (m, 1H), 3.86 (dd, J = 10.5, 5.8 Hz, |
| | | | | | 1H), 3.81 (s, 6H), 3.66 (dd, J = 12.1, |
| | | | | | 6.0 Hz, 1H), |
| | | | | | 3.61-3.37 (m, 6H), 2.34-2.15 (m, 6H), |
| | | | | | 2.07-1.98 (m, 1H), |
| | | | | | 1.98-1.89 (m, 1H), 1.06-0.92 (2 × t, J = 7.5-7.6 Hz, |
| | | | | | 6H). Mixture of two |
| | | | | | diasteroisomeric conformers |
| 165 | 150 | 447.54 | N-(1- | HPLC | 1H NMR (400 MHz, DMSO) δ |
| | | | benzoylpyrrolidin-3- | 99.8%; | 8.65 (2 × d, J = 1.7 Hz, 2H), |
| | | | yl)-8-(1-methyl-1H- | m/z = 448.2 | 8.48 (2 × d, J = 1.8 Hz, 2H), |
| | | | indol-6-yl)quinoxalin- | [M + H]⁺ | 7.69-7.32 (m, 18H), 7.31-7.23 (m, |
| | | | 6-amine | | 2H), 7.05-6.77 (m, 4H), |
| | | | | | 6.52-6.44 (m, 2H), 4.35-4.20 (m, |
| | | | | | 2H), 3.94-3.87 (m, 2H), |
| | | | | | 3.86-3.78 (2 × s, 6H), 3.78-3.50 (m, |
| | | | | | 5H), 3.41-3.35 (m, 1H), |
| | | | | | 2.36-2.21 (m, 2H), 2.09-1.94 (m, |
| | | | | | 2H). |
| 166 | 151 | 413.53 | 2-methyl-1-(3-{[8-(1- | HPLC | 1H NMR (400 MHz, DMSO) ? |
| | | | methyl-1H-indol-6- | 97.2%; | 8.72-8.60 (m, 2H), 8.49 (t, J = 2.2 Hz, |
| | | | yl)quinoxalin-6- | m/z = 414.2 | 2H), 7.70-7.52 (m, |
| | | | yl]amino}pyrrolidin- | [M + H]⁺ | 4H), 7.39 (dd, J = 6.7, 3.1 Hz, |
| | | | 1-yl)propan-1-one | | 4H), 7.28 (ddd, J = 8.2, 2.4, 1.5 Hz, |
| | | | | | 2H), 6.96-6.80 (m, 4H), |
| | | | | | 6.48 (d, J = 3.0 Hz, 2H), |
| | | | | | 4.31 (td, J = 10.9, 6.4 Hz, 1H), |
| | | | | | 4.20 (td, J = 10.2, 5.8 Hz, 1H), |
| | | | | | 3.93 (dd, J = 10.5, 5.8 Hz, 1H), |
| | | | | | 3.82 (s, 6H), 3.75-3.62 (m, 3H), |
| | | | | | 3.55-3.38 (m, 4H), |
| | | | | | 2.75-2.62 (m, 2H), 2.37-2.16 (m, 2H), |
| | | | | | 2.08-1.90 (m, 2H), |
| | | | | | 1.06-0.94 (m, 12H). Mixture of two |
| | | | | | diastereoisomeric conformers. |
| 167 | 152 | 420.52 | 8-(1-methyl-1H- | HPLC | 1H NMR (400 MHz, DMSO) ? |
| | | | indol-6-yl)-N-[1- | 93.9%; | 8.66 (d, J = 1.9 Hz, 1H), 8.48 (d, |
| | | | (pyridin-3- | m/z = 421.2 | J = 1.9 Hz, 1H), 7.99 (d, J = 2.9 Hz, |
| | | | yl)pyrrolidin-3- | [M + H]⁺ | 1H), 7.85 (dd, J = 4.5, 1.0 Hz, |
| | | | yl]quinoxalin-6- | | 1H), 7.63 (s, 1H), 7.59 (d, J = 8.2 Hz, |
| | | | amine | | 1H), 7.38 (dd, J = 5.4, |
| | | | | | 2.8 Hz, 2H), 7.27 (dd, J = 8.2, |
| | | | | | 1.4 Hz, 1H), 7.16 (dd, J = 8.4, |
| | | | | | 4.6 Hz, 1H), 7.00-6.89 (m, |
| | | | | | 3H), 6.46 (dd, J = 3.0, 0.6 Hz, |
| | | | | | 1H), 4.44-4.36 (m, 1H), |
| | | | | | 3.80 (s, 3H), 3.75 (dd, J = 9.9, 5.9 Hz, |
| | | | | | 1H), 3.55-3.47 (m, 1H), |
| | | | | | 3.47-3.38 (m, 1H), |
| | | | | | 2.45-2.36 (m, 1H), 2.16-2.06 (m, 1H) + |
| | | | | | peak partially covered by |
| | | | | | signal of water of 1H |
| 168 | 153 | 413.53 | 1-(4-{[8-(1-methyl- | HPLC | 1H NMR (400 MHz, DMSO) δ |
| | | | 1H-indol-6- | 97.2%; | 8.61 (ddd, J = 6.6, 5.7, 2.0 Hz, |
| | | | yl)quinoxalin-6- | m/z = 414.2 | 4H), 7.73 (dd, J = 8.2, 1.0 Hz, |
| | | | yl]amino}azepan-1- | [M + H]⁺ | 2H), 7.60 (s, 2H), 7.36 (dd, J = 8.2, |
| | | | yl)ethan-1-one | | 1.4 Hz, 2H), |
| | | | | | 7.23-7.19 (m, 2H), 7.13 (dd, J = 3.1, 1.7 Hz, |
| | | | | | 2H), 7.00 (s, 2H), |
| | | | | | 6.57-6.53 (m, 2H), 4.37 (s, 1H), |
| | | | | | 4.04-3.93 (m, 1H), 3.85 (s, 6H), |
| | | | | | 3.69 (ddd, J = 14.0, 11.7, 7.2 Hz, |
| | | | | | 4H), 3.60-3.47 (m, 3H), |
| | | | | | 3.43-3.33 (m, 1H), |
| | | | | | 2.44-2.28 (m, 2H), 2.28-2.15 (m, |
| | | | | | 8H), 2.08-2.00 (m, 2H), |
| | | | | | 1.80 (ddd, J = 19.0, 9.0, 4.6 Hz, 4H), |
| | | | | | 1.72-1.60 (m, 4H). |
| | | | | | Compound is a mixture of 2 |
| | | | | | diastereoisomeric conformers |
| 169 | 154 | 439.56 | N-(1- | HPLC | 1H NMR (400 MHz, δ) δ |
| | | | cyclopropanecarbonylazepan- | 96.2%; | 8.62 (dd, J = 8.1, 1.9 Hz, 2H), |
| | | | 4-yl)-8-(1- | m/z = 440.2 | 8.59 (dd, J = 3.7, 2.0 Hz, 2H), |
| | | | methyl-1H-indol-6- | [M + H]⁺ | 7.73 (d, J = 8.1 Hz, 2H), 7.60 (s, 2H), |
| | | | yl)quinoxalin-6- | | 7.36 (dd, J = 8.2, 1.4 Hz, 2H), |
| | | | amine | | 7.20 (dd, J = 7.1, 2.6 Hz, 2H), |
| | | | | | 7.13 (d, J = 3.0 Hz, 2H), 7.00 (d, |
| | | | | | J = 5.6 Hz, 2H), 6.55 (d, J = 3.0 Hz, |
| | | | | | 2H), 4.37 (s, 2H), |
| | | | | | 4.04-3.89 (m, 2H), 3.85 (s, 6H), |
| | | | | | 3.83-3.70 (m, 5H), 3.69-3.57 (m, |
| | | | | | 2H), 3.47-3.36 (m, 1H), |
| | | | | | 2.50-1.62 (m, 14H), 1.15-0.96 (m, |
| | | | | | 4H), 0.87-0.78 (m, 4H). |
| | | | | | Compound is a mixture of 2 |
| | | | | | diastereoisomeric conformers |
| 170 | 155 | 400.49 | 2-amino-1-[(3S)-3- | HPLC | 1H NMR (400 MHz, DMSO) δ |
| | | | {[8-(1-methyl-1H- | 94.3%; | 8.67 (d, J = 1.4 Hz, 1H), |
| | | | indol-6-yl)quinoxalin- | m/z = 401.3 | 8.53-8.37 (m, 1H), 7.64 (s, 1H), |
| | | | 6- | [M + H]⁺ | 7.60 (d, J = 8.2 Hz, 1H), |
| | | | yl]amino}pyrrolidin- | | 7.40-7.36 (m, 2H), 7.28 (dd, J = 7.8, 1.9 Hz, |
| | | | 1-yl]ethan-1-one | | 1H), 6.94-6.86 (m, 2H), |
| | | | | | 6.48 (d, J = 3.0 Hz, 1H), |
| | | | | | 4.34-4.18 (m, 1H), 3.86-3.79 (m, |
| | | | | | 3H), 3.71 (dd, J = 13.4, 6.2 Hz, |
| | | | | | 1H), 3.60-3.38 (m, 4H), |
| | | | | | 2.32-2.17 (m, 1H), 2.10-1.91 (m, |
| | | | | | 1H). Amine NH2 protons are |
| | | | | | not visible. Mixture of two, |
| | | | | | conformers |
| 171 | 156 | 421.51 | 8-(1-methyl-1H- | HPLC | 1H NMR (400 MHz, DMSO) δ |
| | | | indol-6-yl)-N-[(3S)-1- | 93.5%; | 8.67 (d, J = 1.9 Hz, 1H), 8.49 (d, |
| | | | (pyrimidin-5- | m/z = 422.2 | J = 1.9 Hz, 1H), 8.47 (s, 1H), |
| | | | yl)pyrrolidin-3- | [M + H]⁺ | 8.18 (s, 2H), 7.64 (s, 1H), |
| | | | yl]quinoxalin-6- | | 7.60 (d, J = 8.2 Hz, 1H), |
| | | | amine | | 7.40-7.37 (m, 2H), 7.28 (dd, J = 8.2, 1.4 Hz, |
| | | | | | 1H), 6.97-6.93 (m, 2H), |
| | | | | | 6.47 (dd, J = 3.0, 0.7 Hz, 1H), |
| | | | | | 4.45-4.39 (m, 1H), |
| | | | | | 3.83-3.76 (m, 4H), 3.57-3.42 (m, |
| | | | | | 2H), 3.38-3.33 (m, 42H), |
| | | | | | 2.45-2.36 (m, 1H), 2.13 (td, J = 12.1, |
| | | | | | 4.8 Hz, 1H). |
| 172 | 157 | 357.46 | 8-(1-methyl-1H- | HPLC | 1H NMR (400 MHz, DMSO) δ |
| | | | indol-6-yl)-N-[(3S)-1- | 95.2%; | 8.63 (d, J = 1.9 Hz, 1H), 8.45 (d, |
| | | | methylpyrrolidin-3- | m/z = 358.2 | J = 1.9 Hz, 1H), 7.64-7.58 (m, |
| | | | yl]quinoxalin-6- | [M + H]⁺ | 2H), 7.38 (t, J = 3.0 Hz, 2H), |
| | | | amine | | 7.26 (dd, J = 8.1, 1.4 Hz, 1H), |
| | | | | | 6.82 (d, J = 6.6 Hz, 1H), 6.74 (d, |
| | | | | | J = 2.5 Hz, 1H), 6.48 (d, J = 3.1 Hz, |
| | | | | | 1H), 4.13-4.03 (m, 1H), |
| | | | | | 3.82 (s, 2H), 2.81 (dd, J = 9.3, |
| | | | | | 6.7 Hz, 1H), 2.66 (dd, J = 11.3, |
| | | | | | 5.0 Hz, 1H), 2.55-2.52 (m, |
| | | | | | 1H), 2.42 (dd, J = 14.6, 8.1 Hz, |
| | | | | | 1H), 2.38-2.32 (m, 1H), |
| | | | | | 2.37-2.31 (m, 1H), 2.29 (s, 2H), |
| | | | | | 1.72 (dt, J = 12.7, 7.8 Hz, 1H). |
| 173 | 158 | 413.52 | N-[(1,4-cis)-4-{[8-(1- | HPLC | 1H NMR (400 MHz, DMSO) ? |
| | | | methyl-1H-indol-6- | 99.7%; | 8.62 (d, J = 1.8 Hz, 1H), 8.43 (d, |
| | | | yl)quinoxalin-6- | m/z = 414.3 | J = 1.8 Hz, 1H), 7.77 (d, J = 6.9 Hz, |
| | | | yl]amino}cyclohexyl]acetamide | [M + H]⁺ | 1H), 7.65-7.58 (m, 2H), |
| | | | | | 7.47 (d, J = 2.5 Hz, 1H), 7.39 (d, |
| | | | | | J = 3.0 Hz, 1H), 7.27 (d, J = 9.3 Hz, |
| | | | | | 1H), 6.80 (d, J = 2.3 Hz, 1H), |
| | | | | | 6.55 (d, J = 6.3 Hz, 1H), 6.48 (d, |
| | | | | | J = 2.8 Hz, 1H), 3.82 (s, 3H), |
| | | | | | 3.71 (dd, J = 11.0, 5.6 Hz, 1H), |
| | | | | | 3.58 (dd, J = 11.0, 6.5 Hz, 1H), |
| | | | | | 1.83 (s, 3H), 1.81-1.72 (m, 4H), |
| | | | | | 1.70-1.60 (m, 4H). |
| 178 | 162 | 434.55 | 8-(1-methyl-1H- | HPLC | 1H NMR (400 MHz, DMSO) δ |
| | | | indol-6-yl)-N-[(3S)-1- | 97.8%; | 8.65 (d, J = 1.8 Hz, 1H), 8.47 (d, |
| | | | (3-methylpyridin-2- | m/z = 435.3 | J = 1.9 Hz, 1H), 7.99-7.95 (m, |
| | | | yl)pyrrolidin-3- | [M + H]⁺ | 1H), 7.67-7.53 (m, 1H), |
| | | | yl]quinoxalin-6- | | 7.40 (dd, J = 9.9, 2.8 Hz, 1H), |
| | | | amine | | 7.35 (d, J = 6.2 Hz, 1H), 7.27 (dd, J = 8.2, |
| | | | | | 1.4 Hz, 1H), 6.92 (d, J = 6.4 Hz, |
| | | | | | 1H), 6.89 (d, J = 2.4 Hz, 1H), |
| | | | | | 6.66 (dd, J = 7.2, 4.9 Hz, 1H), |
| | | | | | 6.48 (d, J = 3.0 Hz, 1H), |
| | | | | | 4.27 (dd, J = 10.4, 5.5 Hz, 1H), |
| | | | | | 3.94 (dd, J = 10.6, 5.9 Hz, 1H), |
| | | | | | 3.81 (s, 3H), 3.73 (q, J = 7.3 Hz, 1H), |
| | | | | | 3.61 (dd, J = 14.4, 8.9 Hz, 1H), |
| | | | | | 3.50 (dd, J = 10.7, 4.1 Hz, 1H), |
| | | | | | 2.38-2.27 (m, 4H), |
| | | | | | 2.05-1.94 (m, 1H). |
| 179 | 163 | 421.51 | 8-(1-methyl-1H- | HPLC | Not determined |
| | | | indol-6-yl)-N-[(3S)-1- | 94.6%; | |
| | | | (pyrazin-2- | m/z = 422.3 | |
| | | | yl)pyrrolidin-3- | [M + H]⁺ | |
| | | | yl]quinoxalin-6- | | |
| | | | amine | | |
| 180 | 164 | 435.54 | 8-(1-methyl-1H- | HPLC | 1H NMR (400 MHz, DMSO) δ |
| | | | indol-6-yl)-N-[(3S)-1- | 95.5%; | 8.67 (d, J = 1.9 Hz, 1H), 8.49 (d, |
| | | | (2-methylpyrimidin- | m/z = 436.3 | J = 1.9 Hz, 1H), 8.06 (d, J = 5.9 Hz, |
| | | | 4-yl)pyrrolidin-3- | [M + H]⁺ | 1H), 7.63 (s, 1H), 7.60 (d, J = 8.2 Hz, |
| | | | yl]quinoxalin-6- | | 1H), 7.38 (d, J = 3.0 Hz, |
| | | | amine | | 2H), 7.28 (dd, J = 8.2, 1.5 Hz, |
| | | | | | 1H), 6.98-6.90 (m, 2H), |
| | | | | | 6.47 (dd, J = 3.1, 0.8 Hz, 1H), |
| | | | | | 6.36 (d, J = 6.0 Hz, 1H), |
| | | | | | 4.44-4.27 (m, 1H), 3.89-3.75 (m, |
| | | | | | 4H), 3.74-3.44 (m, 3H), |
| | | | | | 2.36 (s, 3H), 2.30-2.03 (m, 2H). |
| | | | | | Signals coming from |
| | | | | | pyrrolidine moiety are given as |
| | | | | | broader multiples. |
| 181 | 165 | 421.51 | 8-(1-methyl-1H- | HPLC | 1H NMR (400 MHz, DMSO) δ |
| | | | indol-6-yl)-N-[(3S)-1- | 99.2%; | 8.67 (d, J = 1.9 Hz, 1H), 8.49 (d, |
| | | | (pyrimidin-4- | m/z = 422.2 | J = 1.8 Hz, 2H), 8.16 (d, J = 6.1 Hz, |
| | | | yl)pyrrolidin-3- | [M + H]⁺ | 1H), 7.64 (s, 1H), 7.60 (d, J = 8.2 Hz, |
| | | | yl]quinoxalin-6- | | 1H), 7.42-7.35 (m, |
| | | | amine | | 2H), 7.28 (dd, J = 8.2, 1.3 Hz, |
| | | | | | 1H), 6.95 (dd, J = 11.9, 4.2 Hz, |
| | | | | | 2H), 6.58 (d, J = 6.2 Hz, 1H), |
| | | | | | 6.47 (d, J = 3.0 Hz, 1H), |
| | | | | | 4.48-4.31 (m, 1H), 3.95-3.80 (m, |
| | | | | | 4H), 3.79-3.48 (m, 3H), |
| | | | | | 2.45-2.29 (m, 1H), 2.21-2.04 (m, |
| | | | | | 1H). Signals coming from |
| | | | | | pyrrolidine moiety are given as |
| | | | | | broader multiples. |
| 182 | 166 | 421.51 | 8-(1-methyl-1H- | HPLC | 1H NMR (400 MHz, DMSO) δ |
| | | | indol-6-yl)-N-[(3S)-1- | 94.5%; | 8.63 (d, J = 1.7 Hz, 2H), |
| | | | (pyrimidin-2- | m/z = 422.2 | 8.49-8.46 (m, 2H), 7.27 (t, J = 2.7 Hz, |
| | | | yl)pyrrolidin-3- | [M + H]⁺ | 2H), 6.99 (s, 2H), 6.94 (d, J = 8.0 Hz, |
| | | | yl]quinoxalin-6- | | 2H), 6.85-6.76 (m, |
| | | | amine | | 4H), 6.74 (d, J = 8.0 Hz, 2H), |
| | | | | | 5.34 (s, 2H), 4.30-4.21 (m, |
| | | | | | 1H), 4.21-4.13 (m, 1H), |
| | | | | | 3.87 (dd, J = 10.5, 5.8 Hz, 1H), |
| | | | | | 3.67-3.57 (m, 3H), 3.50-3.43 (m, |
| | | | | | 2H), 3.38 (dd, J = 10.9, 3.6 Hz, |
| | | | | | 2H), 3.15-3.09 (m, 4H), |
| | | | | | 3.06 (t, J = 5.7 Hz, 4H), 2.83 (s, 6H), |
| | | | | | 2.32-2.25 (m, 1H), |
| | | | | | 2.25-2.15 (m, 1H), 2.05-1.87 (m, |
| | | | | | 8H), 1.83-1.75 (m, 4H). |
| 183 | 167 | 437.51 | 4-[(3S)-3-{[8-(1- | HPLC | 1H NMR (400 MHz, DMSO) δ |
| | | | methyl-1H-indol-6- | 94.3%; | 10.44 (bs, 1H), 8.67 (d, J = 1.9 Hz, |
| | | | yl)quinoxalin-6- | m/z = 438.3 | 1H), 8.49 (d, J = 1.8 Hz, 1H), |
| | | | yl]amino}pyrrolidin- | [M + H]⁺ | 7.64 (s, 1H), 7.60 (d, J = 8.2 Hz, |
| | | | 1-yl]pyrimidin-2-ol | | 1H), 7.47-7.41 (m, 1H), |
| | | | | | 7.38 (d, J = 2.9 Hz, 2H), 7.28 (d, J = 8.2 Hz, |
| | | | | | 1H), 6.97-6.92 (m, |
| | | | | | 2H), 6.48 (d, J = 3.0 Hz, 1H), |
| | | | | | 5.77 (d, J = 5.2 Hz, 1H), |
| | | | | | 4.41-4.25 (m, 1H), 3.88-3.74 (m, |
| | | | | | 4H), 3.68-3.56 (m, 3H), |
| | | | | | 3.46-3.38 (m, 1H), 2.41-2.25 (m, |
| | | | | | 1H), 2.14-1.99 (m, 1H). |
| 184 | 168 | 435.54 | 8-(1-methyl-1H- | HPLC | 1H NMR (400 MHz, DMSO) δ |
| | | | indol-6-yl)-N-[1- | 99.7%; | 8.64 (d, J = 1.9 Hz, 1H), 8.45 (d, |
| | | | (pyrimidin-2- | m/z = 436.2 | J = 1.9 Hz, 1H), 8.37 (d, J = 4.7 Hz, |
| | | | yl)piperidin-4- | [M + H]⁺ | 2H), 7.63 (s, 1H), 7.60 (d, J = 8.2 Hz, |
| | | | yl]quinoxalin-6- | | 1H), 7.38 (d, J = 3.1 Hz, |
| | | | amine | | 1H), 7.37 (d, J = 2.6 Hz, 1H), |
| | | | | | 7.27 (dd, J = 8.2, 1.4 Hz, 1H), |
| | | | | | 6.93 (d, J = 2.5 Hz, 1H), |
| | | | | | 6.65-6.59 (m, 2H), 6.48 (dd, J = 3.1, |
| | | | | | 0.7 Hz, 1H), 4.59 (dt, J = 6.6, |
| | | | | | 3.6 Hz, 2H), 3.88-3.76 (m, 4H), |
| | | | | | 3.30-3.21 (m, 2H), 2.10 (dd, J = 12.7, |
| | | | | | 2.7 Hz, 2H), 1.49-1.36 (m, |
| | | | | | 2H). |
| 185 | 169 | 419.53 | 8-(1-methyl-1H- | HPLC | 1H NMR (400 MHz, DMSO) δ |
| | | | indol-6-yl)-N-(1- | 95.5%; | 8.66 (d, J = 1.9 Hz, 1H), 8.48 (d, |
| | | | phenylpyrrolidin-3- | m/z = 420.2 | J = 1.9 Hz, 1H), 7.63 (s, 1H), |
| | | | yl)quinoxalin-6- | [M + H]⁺ | 7.60 (d, J = 8.2 Hz, 1H), 7.40 (d, |
| | | | amine | | J = 2.6 Hz, 1H), 7.38 (d, J = 3.1 Hz, |
| | | | | | 1H), 7.28 (dd, J = 8.2, 1.4 Hz, |
| | | | | | 1H), 7.18 (dd, J = 8.6, 7.3 Hz, |
| | | | | | 2H), 6.96 (d, J = 6.6 Hz, 1H), |
| | | | | | 6.92 (d, J = 2.6 Hz, 1H), |
| | | | | | 6.63-6.54 (m, 3H), 6.47 (dd, J = 3.1, |
| | | | | | 0.8 Hz, 1H), 4.43-4.31 (m, |
| | | | | | 1H), 3.81 (s, 3H), 3.72 (dd, J = 9.7, |
| | | | | | 6.0 Hz, 1H), |
| | | | | | 3.52-3.41 (m, 1H), 3.41-3.34 (m, 1H), |
| | | | | | 3.27 (dd, J = 9.7, 3.3 Hz, 1H), |
| | | | | | 2.45-2.30 (m, 1H), |
| | | | | | 2.17-2.03 (m, 1H). |
| 186 | 170 | 421.51 | 8-(3-methyl-1H- | HPLC | 1H NMR (400 MHz, DMSO) δ |
| | | | indol-5-yl)-N-[(3S)-1- | 93.8%; | 8.67 (d, J = 1.9 Hz, 1H), 8.49 (t, |
| | | | (pyrimidin-4- | m/z = 422.2 | J = 4.4 Hz, 2H), 8.15 (d, J = 5.7 Hz, |
| | | | yl)pyrrolidin-3- | [M + H]⁺ | 1H), 7.64 (s, 1H), 7.60 (d, J = 8.2 Hz, |
| | | | yl]quinoxalin-6- | | 1H), 7.41-7.35 (m, |
| | | | amine | | 2H), 7.28 (dd, J = 8.2, 1.4 Hz, |
| | | | | | 1H), 6.99-6.90 (m, 2H), |
| | | | | | 6.56 (d, J = 6.1 Hz, 1H), 6.47 (d, J = 3.0 Hz, |
| | | | | | 1H), 4.47-4.31 (m, |
| | | | | | 1H), 3.90-3.76 (m, 4H), |
| | | | | | 3.76-3.49 (m, 2H), 2.44-2.31 (m, |
| | | | | | 1H), 2.19-2.06 (m, 1H). |
| | | | | | Signals coming from |
| | | | | | pyrrolidine moiety are given as |
| | | | | | broader multiples. |
| 187 | 171 | 435.54 | 8-(1,3-dimethyl-1H- | HPLC | 1H NMR (400 MHz, DMSO) ? |
| | | | indol-5-yl)-N-[(3S)-1- | 99.5%; | 8.66 (d, J = 1.9 Hz, 1H), 8.48 (d, |
| | | | (pyrimidin-2- | m/z = 436.3 | J = 1.9 Hz, 1H), 8.35 (d, J = 4.7 Hz, |
| | | | yl)pyrrolidin-3- | [M + H]⁺ | 2H), 7.68 (d, J = 1.4 Hz, 1H), |
| | | | yl]quinoxalin-6- | | 7.46-7.35 (m, 3H), 7.13 (d, J = 1.0 Hz, |
| | | | amine | | 1H), 6.94 (d, J = 6.3 Hz, |
| | | | | | 1H), 6.89 (d, J = 2.5 Hz, 1H), |
| | | | | | 6.61 (t, J = 4.8 Hz, 1H), |
| | | | | | 4.40-4.28 (m, 1H), 3.88 (dd, J = 11.5, |
| | | | | | 5.7 Hz, 1H), 3.77 (s, 3H), |
| | | | | | 3.72-3.57 (m, 3H), 2.38 (dt, J = 13.9, |
| | | | | | 8.0 Hz, 1H), 2.27 (d, J = 0.9 Hz, |
| | | | | | 3H), 2.10 (td, J = 11.6, 5.5 Hz, |
| | | | | | 1H). |
| 188 | 172 | 381.44 | N-[(2- | HPLC | 1H NMR (400 MHz, DMSO) δ |
| | | | aminopyrimidin-5- | 98.4%; | 8.62 (d, J = 1.9 Hz, 1H), 8.46 (d, |
| | | | yl)methyl]-8-(1- | m/z = 382.2 | J = 1.9 Hz, 1H), 8.32 (s, 2H), |
| | | | methyl-1H-indol-5- | [M + H]⁺ | 7.75 (d, J = 1.0 Hz, 1H), 7.50 (d, |
| | | | yl)quinoxalin-6- | | J = 8.5 Hz, 1H), 7.41-7.33 (m, |
| | | | amine | | 3H), 7.01 (t, J = 5.4 Hz, 1H), |
| | | | | | 6.84 (d, J = 2.6 Hz, 1H), 6.58 (s, |
| | | | | | 2H), 6.48 (d, J = 3.0 Hz, 1H), |
| | | | | | 4.25 (d, J = 5.4 Hz, 2H), 3.84 (s, |
| | | | | | 3H). |
| 189 | 173 | 445.32 | N-[(5-bromopyridin- | HPLC | 1H NMR (400 MHz, DMSO) δ |
| | | | 3-yl)methyl]-8-(1- | 97.8%; | 8.69 (d, J = 1.7 Hz, 1H), |
| | | | methyl-1H-indazol-6- | m/z = 455.2 | 8.64 (dd, J = 11.7, 2.1 Hz, 2H), |
| | | | yl)quinoxalin-6- | [M + H]⁺ | 8.48 (d, J = 1.9 Hz, 1H), 8.13 (t, J = 2.0 Hz, |
| | | | amine | | 1H), 8.10 (d, J = 0.8 Hz, |
| | | | | | 1H), 7.83-7.77 (m, 2H), |
| | | | | | 7.46 (d, J = 2.6 Hz, 1H), |
| | | | | | 7.39-7.32 (m, 2H), 6.86 (d, J = 2.6 Hz, |
| | | | | | 1H), 4.57 (d, J = 5.9 Hz, 2H), |
| | | | | | 4.07 (s, 3H). |
| 190 | 174 | 389.50 | 1-[4-({8-[3- | HPLC | 1H NMR (400 MHz, DMSO) δ |
| | | | (dimethylamino)phenyl]quinoxalin- | 97.9%; | 8.62 (d, J = 1.6 Hz, 1H), 8.43 (d, |
| | | | 6- | m/z = 390.5 | J = 1.8 Hz, 1H), 7.29 (d, J = 2.4 Hz, |
| | | | yl}amino)piperidin-1- | [M + H]⁺ | 1H), 7.25 (t, J = 7.9 Hz, 1H), |
| | | | yl]ethan-1-one | | 6.93-6.86 (m, 2H), 6.84 (d, J = 7.6 Hz, |
| | | | | | 1H), 6.77 (dd, J = 8.4, |
| | | | | | 2.1 Hz, 1H), 6.60 (d, J = 7.6 Hz, |
| | | | | | 1H), 4.26 (d, J = 13.0 Hz, 1H), |
| | | | | | 3.82 (d, J = 13.6 Hz, 1H), |
| | | | | | 3.79-3.62 (m, 1H), 3.32-3.20 (m, |
| | | | | | 1H), 2.96-2.82 (m, 7H), |
| | | | | | 2.12-1.94 (m, 5H), 1.46-1.25 (m, |
| | | | | | 2H). |
| 191 | 175 | 375.43 | 3-{7-[(1- | HPLC | 1H NMR (400 MHz, DMSO) δ |
| | | | acetylpyrrolidin-3- | 99.8%; | 8.69 (d, J = 1.5 Hz, 1H), |
| | | | yl)amino]quinoxalin- | m/z = 376.2 | 8.54-8.48 (m, 1H), 8.11 (s, 1H), |
| | | | 5-yl}benzamide | [M + H]⁺ | 8.03 (s, 1H), 7.93 (d, J = 7.4 Hz, 1H), |
| | | | | | 7.77 (d, J = 7.6 Hz, 1H), 7.55 (t, |
| | | | | | J = 7.6 Hz, 1H), 7.44-7.34 (m, |
| | | | | | 2H), 6.93 (dt, J = 9.0, 5.3 Hz, |
| | | | | | 2H), 4.36-4.16 (m, 1H), |
| | | | | | 3.92-3.57 (m, 2H), 3.49-3.43 (m, |
| | | | | | 1H), 3.42-3.39 (m, 1H), |
| | | | | | 2.34-2.15 (m, 1H), 2.08-1.85 (m, |
| | | | | | 4H). Mixture of two |
| | | | | | conformers |
| 192 | 176 | 389.46 | 1-(5-{7-[(1- | HPLC | 1H NMR (400 MHz, DMSO) δ |
| | | | acetylpiperidin-4- | 93.3%; | 8.95 (d, J = 1.8 Hz, 1H), 8.71 (d, |
| | | | yl)amino]quinoxalin- | m/z = 390.2 | J = 1.7 Hz, 1H), 8.49 (d, J = 1.7 Hz, |
| | | | 5-yl}pyridin-2- | [M + H]⁺ | 1H), 8.22 (dd, J = 8.1, 2.0 Hz, |
| | | | yl)ethan-1-one | | 1H), 8.08 (d, J = 8.1 Hz, 1H), |
| | | | | | 7.45 (d, J = 2.3 Hz, 1H), 7.02 (d, |
| | | | | | J = 2.3 Hz, 1H), 6.74 (d, J = 7.7 Hz, |
| | | | | | 1H), 4.28 (d, J = 12.4 Hz, |
| | | | | | 1H), 3.90-3.71 (m, 2H), |
| | | | | | 3.31-3.22 (m, 1H), 2.97-2.83 (m, |
| | | | | | 1H), 2.71 (s, 3H), |
| | | | | | 2.09-1.97 (m, 5H), 1.46-1.27 (m, 2H). |
| 193 | 177 | 389.50 | 1-[(3S)-3-({8-[3- | HPLC | 1H NMR (400 MHz, DMSO) δ |
| | | | (dimethylamino)-4- | 96.6%; | 8.66 (d, J = 1.6 Hz, 1H), |
| | | | methylphenyl]quinoxalin- | m/z = 390.2 | 8.47 (dd, J = 1.8, 1.0 Hz, 1H), 7.31 (t, |
| | | | 6- | [M + H]⁺ | J = 2.9 Hz, 1H), 7.22 (d, J = 7.3 Hz, |
| | | | yl}amino)pyrrolidin- | | 2H), 7.19-7.13 (m, 1H), |
| | | | 1-yl]ethan-1-one | | 6.93-6.82 (m, 2H), |
| | | | | | 4.31-4.15 (m, 1H), 3.88 (dd, J = 10.5, |
| | | | | | 5.9 Hz, 1H), 3.69-3.56 (m, |
| | | | | | 1H), 3.46 (t, J = 7.0 Hz, 1H), |
| | | | | | 3.39 (dd, J = 11.2, 3.5 Hz, 1H), |
| | | | | | 2.68 (s, 6H), 2.56-2.52 (m, |
| | | | | | 1H), 2.33 (s, 3H), |
| | | | | | 2.32-2.15 (m, 1H), 2.06-1.88 (m, 4H). |
| | | | | | Mixture of two diastereomeric |
| | | | | | conformers. |
| 194 | 178 | 405.50 | 1-[(3S)-3-({8-[3- | HPLC | 1H NMR (400 MHz, DMSO) δ |
| | | | (dimethylamino)-4- | 95.8%; | 8.65 (d, J = 1.7 Hz, 1H), |
| | | | methoxyphenyl]quinoxalin- | m/z = 406.2 | 8.48 (dd, J = 1.8, 0.9 Hz, 1H), 7.30 (t, |
| | | | 6- | [M + H]⁺ | J = 2.9 Hz, 1H), 7.18 (dt, J = 8.3, |
| | | | yl}amino)pyrrolidin- | | 2.1 Hz, 1H), 7.09 (d, J = 1.3 Hz, |
| | | | 1-yl]ethan-1-one | | 1H), 7.02 (d, J = 8.3 Hz, 1H), |
| | | | | | 6.85 (dt, J = 8.3, 5.2 Hz, 2H), |
| | | | | | 4.32-4.14 (m, 1H), |
| | | | | | 3.91-3.83 (m, 3.5H), 3.69-3.56 (m, |
| | | | | | 1.5H), 3.47 (dd, J = 10.1, 4.9 Hz, |
| | | | | | 1H), 3.39 (dd, J = 11.1, 3.6 Hz, |
| | | | | | 1H), 2.73 (s, 6H), |
| | | | | | 2.56-2.52 (m, 1H), 2.32-2.14 (m, |
| | | | | | 1H), 2.07-1.88 (m, 4H). |
| | | | | | mixture of two conformers |
| 195 | 179 | 375.48 | 1-[(3S)-3-({8-[4- | HPLC | 1H NMR (400 MHz, DMSO) δ |
| | | | methyl-3- | 98.7%; | 8.64 (d, J = 1.6 Hz, 1H), |
| | | | (methylamino)phenyl]quinoxalin- | m/z = 376.2 | 8.46 (dd, J = 1.8, 1.1 Hz, 1H), |
| | | | 6- | [M + H]⁺ | 7.33-7.28 (m, 1H), 7.03 (d, J = 7.5 Hz, |
| | | | yl}amino)pyrrolidin- | | 1H), 6.91-6.80 (m, 1H), |
| | | | 1-yl]ethan-1-one | | 6.72 (dd, J = 7.4, 2.1 Hz, 1H), |
| | | | | | 6.66 (s, 1H), 5.07 (d, J = 5.1 Hz, |
| | | | | | 1H), 4.22 (d, J = 30.8 Hz, 1H), |
| | | | | | 3.88 (dd, J = 10.6, 5.8 Hz, 1H), |
| | | | | | 3.69-3.56 (m, 1H), 3.46 (t, J = 6.2 Hz, |
| | | | | | 1H), 3.42-3.35 (m, |
| | | | | | 1H), 2.28-2.17 (m, 1H), |
| | | | | | 2.14 (s, 1H), 2.07-1.88 (m, 1H). |
| | | | | | Mixture of two conformers |
| 196 | 180 | 403.53 | 1-[(3S)-3-[(8-{3- | HPLC | Not determined |
| | | | [ethyl(methyl)amino]- | 94.2%; | |
| | | | 4- | m/z = 404.3 | |
| | | | methylphenyl}quinoxalin- | [M + H]⁺ | |
| | | | 6- | | |
| | | | yl)amino]pyrrolidin- | | |
| | | | 1-yl]ethan-1-one | | |
| 197 | 181 | 377.45 | 1-(4-{[8-(2- | HPLC | 1H NMR (400 MHz, DMSO) δ |
| | | | methoxypyridin-4- | 96.5%; | 8.68 (d, J = 1.9 Hz, 1H), 8.48 (d, |
| | | | yl)quinoxalin-6- | m/z = 378.2 | J = 1.9 Hz, 1H), 8.25 (d, J = 5.3 Hz, |
| | | | yl]amino}piperidin-1- | [M + H]⁺ | 1H), 7.39 (d, J = 2.5 Hz, 1H), |
| | | | yl)ethan-1-one | | 7.20 (dd, J = 5.3, 1.3 Hz, 1H), |
| | | | | | 7.03 (s, 1H), 6.98 (d, J = 2.4 Hz, |
| | | | | | 1H), 6.67 (d, J = 7.8 Hz, 1H), |
| | | | | | 4.26 (d, J = 13.3 Hz, 1H), |
| | | | | | 3.90 (d, J = 13.6 Hz, 3H), 3.79 (dd, J = 27.8, |
| | | | | | 11.1 Hz, 2H), 3.26 (d, J = 11.2 Hz, |
| | | | | | 1H), 2.90 (t, J = 10.9 Hz, |
| | | | | | 1H), 2.11-1.93 (m, 5H), |
| | | | | | 1.35 (ddd, J = 46.8, 23.3, 13.3 Hz, |
| | | | | | 3H). |
| 198 | 182 | 387.49 | 1-[(3S)-3-{[8-(1- | HPLC | 1H NMR (400 MHz, DMSO) δ |
| | | | methyl-2,3-dihydro- | 95.4%; | 8.65 (dd, J = 1.9, 0.8 Hz, 1H), |
| | | | 1H-indol-6- | m/z = 388.2 | 8.47 (dd, J = 2.0, 1.2 Hz, 1H), |
| | | | yl)quinoxalin-6- | [M + H]⁺ | 7.28 (dd, J = 3.9, 2.6 Hz, 1H), |
| | | | yl]amino}pyrrolidin- | | 7.11 (dd, J = 7.5, 1.2 Hz, 1H), |
| | | | 1-yl]ethan-1-one | | 6.92-6.77 (m, 3H), |
| | | | | | 6.69-6.65 (m, 1H), 4.32-4.11 (m, |
| | | | | | 1H), 3.88 (dd, J = 10.5, 5.8 Hz, |
| | | | | | 0.5H), 3.67-3.55 (m, 1.5H), |
| | | | | | 3.50-3.43 (m, 1H), 3.38 (dd, J = 11.2, |
| | | | | | 3.6 Hz, 1H), |
| | | | | | 3.32-3.25 (m, 2H), 2.93 (t, J = 8.1 Hz, 2H), |
| | | | | | 2.72 (d, J = 0.7 Hz, 3H), |
| | | | | | 2.33-2.14 (m, 1H), 2.06-1.88 (m, |
| | | | | | 4H). Mixture of two |
| | | | | | conformers in 1:1 ratio |
| 199 | 183 | 401.51 | 1-(4-{[8-(1-methyl- | HPLC | 1H NMR (400 MHz, DMSO) δ |
| | | | 2,3-dihydro-1H- | 95.5%; | 8.62 (d, J = 1.9 Hz, 1H), 8.43 (d, |
| | | | indol-6-yl)quinoxalin- | m/z = 402.2 | J = 2.0 Hz, 1H), 7.27 (d, J = 2.6 Hz, |
| | | | 6-yl]amino}piperidin- | [M + H]⁺ | 1H), 7.11 (d, J = 7.5 Hz, 1H), |
| | | | 1-yl)ethan-1-one | | 6.88 (d, J = 2.6 Hz, 1H), |
| | | | | | 6.78 (dd, J = 7.4, 1.5 Hz, 1H), |
| | | | | | 6.66 (d, J = 1.4 Hz, 1H), 6.59 (d, J = 7.7 Hz, |
| | | | | | 1H), 4.27 (d, J = 13.3 Hz, |
| | | | | | 1H), 3.92-3.79 (m, 1H), |
| | | | | | 3.79-3.67 (m, 1H), |
| | | | | | 3.34-3.25 (m, 1H), 2.98-2.86 (m, |
| | | | | | 3H), 2.54-2.52 (m, 1H), |
| | | | | | 2.10-1.94 (m, 2H), 1.44-1.26 (m, J = 43.8, |
| | | | | | 10.8 Hz, 2H). |
| 200 | 184 | 401.51 | 1-(3-{[8-(1-methyl- | HPLC | 1H NMR (400 MHz, DMSO) δ |
| | | | 1,2,3,4- | 98.5%; | 8.64 (s, 2H), 8.45 (dd, J = 1.8, |
| | | | tetrahydroquinolin- | m/z = 402.2 | 1.0 Hz, 2H), 7.31-7.24 (m, |
| | | | 7-yl)quinoxalin-6- | [M + H]⁺ | 2H), 6.95 (d, J = 7.5 Hz, 2H), |
| | | | yl]amino}pyrrolidin- | | 6.91-6.79 (m, 4H), |
| | | | 1-yl)ethan-1-one | | 6.76-6.69 (m, 4H), 4.30-4.21 (m, 1H), |
| | | | | | 4.20-4.12 (m, 1H), 3.87 (dd, J = 10.5, |
| | | | | | 5.8 Hz, 1H), |
| | | | | | 3.68-3.54 (m, 3H), 3.50-3.34 (m, 4H), |
| | | | | | 3.22 (t, J = 5.6 Hz, 4H), 2.83 (s, |
| | | | | | 6H), 2.75 (t, J = 6.4 Hz, 4H), |
| | | | | | 2.34-2.14 (m, 2H), |
| | | | | | 2.05-1.87 (m, 12H). Mixtute of two |
| | | | | | diastereomeric conformers. |
| 201 | 185 | 415.54 | 1-(4-{[8-(1-methyl- | HPLC | 1H NMR (400 MHz, DMSO) δ |
| | | | 1,2,3,4- | 99.1%; | 8.61 (d, J = 1.7 Hz, 1H), 8.42 (d, |
| | | | tetrahydroquinolin- | m/z = 416.3 | J = 1.9 Hz, 1H), 7.27 (d, J = 2.6 Hz, |
| | | | 7-yl)quinoxalin-6- | [M + H]⁺ | 1H), 6.95 (d, J = 7.5 Hz, 1H), |
| | | | yl]amino}piperidin-1- | | 6.86 (d, J = 2.0 Hz, 1H), |
| | | | yl)ethan-1-one | | 6.76-6.67 (m, 2H), 6.57 (d, J = 7.8 Hz, |
| | | | | | 1H), 4.33-4.21 (m, 1H), |
| | | | | | 3.87-3.77 (m, 1H), |
| | | | | | 3.78-3.66 (m, 1H), 3.30-3.17 (m, 3H), |
| | | | | | 2.98-2.81 (m, 4H), 2.75 (t, J = 6.4 Hz, |
| | | | | | 2H), 2.11-1.88 (m, |
| | | | | | 7H), 1.40 (td, J = 13.2, 4.0 Hz, |
| | | | | | 1H), 1.34-1.24 (td, J = 13.2, |
| | | | | | 4.0 Hz, 1H). |
| 202 | 186 | 402.50 | 1-[(3S)-3-{[8-(4- | HPLC | 1H NMR (400 MHz, DMSO) δ |
| | | | methyl-1,2,3,4- | 97.4%; | 8.61 (d, J = 1.7 Hz, 2H), |
| | | | tetrahydroquinoxalin- | m/z = 403.2 | 8.47-8.45 (m, 2H), 7.24 (t, J = 2.8 Hz, |
| | | | 6-yl)quinoxalin-6- | [M + H]⁺ | 2H), 6.83-6.72 (m, 8H), |
| | | | yl]amino}pyrrolidin- | | 6.45 (d, J = 7.8 Hz, 2H), 5.70 (s, 2H), |
| | | | 1-yl]ethan-1-one | | 4.30-4.20 (m, 1H), |
| | | | | | 4.20-4.11 (m, 1H), 3.87 (dd, J = 10.6, |
| | | | | | 5.9 Hz, 1H), 3.67-3.56 (m, |
| | | | | | 3H), 3.46 (ddd, J = 9.5, 6.6, 3.0 Hz, |
| | | | | | 2H), 3.42-3.38 (m, J = 6.4 Hz, |
| | | | | | 5H), 3.38-3.35 (m, 1H), |
| | | | | | 3.19-3.14 (m, 4H), 2.79 (s, |
| | | | | | 6H), 2.32-2.23 (m, 1H), |
| | | | | | 2.23-2.14 (m, 1H), 2.05-1.88 (m, |
| | | | | | 8H). Compound is a |
| | | | | | diastereomeric conformers |
| | | | | | mixture in ca 1:1 ratio. |
| 203 | 187 | 416.53 | 1-[(3S)-3-{[8-(5- | HPLC | 1H NMR (400 MHz, DMSO) δ |
| | | | methyl-2,3,4,5- | 98.0%; | 8.63 (d, J = 1.7 Hz, 2H), |
| | | | tetrahydro-1H-1,5- | m/z = 417.2 | 8.49-8.46 (m, 2H), 7.27 (t, J = 2.7 Hz, |
| | | | benzodiazepin-7- | [M + H]⁺ | 2H), 6.99 (s, 2H), 6.94 (d, J = 8.0 Hz, |
| | | | yl)quinoxalin-6- | | 2H), 6.85-6.76 (m, |
| | | | yl]amino}pyrrolidin- | | 4H), 6.74 (d, J = 8.0 Hz, 2H), |
| | | | 1-yl]ethan-1-one | | 5.34 (s, 2H), 4.30-4.21 (m, |
| | | | | | 1H), 4.21-4.13 (m, 1H), |
| | | | | | 3.87 (dd, J = 10.5, 5.8 Hz, 1H), |
| | | | | | 3.67-3.57 (m, 3H), 3.50-3.43 (m, |
| | | | | | 2H), 3.38 (dd, J = 10.9, 3.6 Hz, |
| | | | | | 2H), 3.15-3.09 (m, 4H), |
| | | | | | 3.06 (t, J = 5.7 Hz, 4H), 2.83 (s, 6H), |
| | | | | | 2.32-2.25 (m, 1H), |
| | | | | | 2.25-2.15 (m, 1H), 2.05-1.87 (m, |
| | | | | | 8H), 1.83-1.75 (m, 4H). Cpd. |
| | | | | | is a diastereomeric conformers |
| | | | | | mixture in ca 1:1 ratio. |
| 204 | 188 | 386.46 | 1-(4-{[8-(1H-1,3- | HPLC | 1H NMR (400 MHz, DMSO) δ |
| | | | benzodiazol-6- | 94.1%; | 12.55 (s, 1H), 8.65 (d, J = 1.9 Hz, |
| | | | yl)quinoxalin-6- | m/z = 387.2 | 1H), 8.46 (d, J = 1.9 Hz, 1H), |
| | | | yl]amino}piperidin-1- | [M + H]⁺ | 8.27 (s, 1H), 7.87-7.56 (m, |
| | | | yl)ethan-1-one | | 2H), 7.47-7.39 (m, 1H), |
| | | | | | 7.38 (d, J = 2.5 Hz, 1H), 6.91 (d, J = 2.5 Hz, |
| | | | | | 1H), 6.62 (d, J = 7.7 Hz, |
| | | | | | 1H), 4.27 (d, J = 12.9 Hz, 1H), |
| | | | | | 3.83 (d, J = 13.9 Hz, 1H), |
| | | | | | 3.79-3.70 (m, 1H), 3.32-2.87 (m, |
| | | | | | 3H), 2.96-2.87 (m, 1H), |
| | | | | | 2.12-1.97 (m, 5H), 1.47-1.27 (m, |
| | | | | | 2H |
| 205 | 189 | 385.47 | 1-(4-{[8-(1H-indol-6- | HPLC | 1H NMR (400 MHz, DMSO) δ |
| | | | yl)quinoxalin-6- | 97.6%; | 11.17 (s, 1H), 8.63 (d, J = 1.7 Hz, |
| | | | yl]amino}piperidin-1- | m/z = 386.2 | 1H), 8.45 (d, J = 1.8 Hz, 1H), |
| | | | yl)ethan-1-one | [M + H]⁺ | 7.65 (s, 1H), 7.60 (d, J = 8.2 Hz, |
| | | | | | 1H), 7.46-7.29 (m, 2H), |
| | | | | | 7.22 (dd, J = 8.1, 1.2 Hz, 1H), |
| | | | | | 6.88 (d, J = 2.3 Hz, 1H), 6.60 (d, J = 7.8 Hz, |
| | | | | | 1H), 6.47 (s, 1H), |
| | | | | | 4.33-4.19 (m, 1H), 3.88-3.67 (m, |
| | | | | | 2H), 3.30-3.21 (m, 1H-part. |
| | | | | | covered by peak of res. water), |
| | | | | | 2.97-2.84 (m, 1H), |
| | | | | | 2.12-1.94 (m, 5H), 1.54-1.24 (m, 2H). |
| 206 | 190 | 400.49 | 1-(4-{[8-(1-methyl- | HPLC | 1H NMR (400 MHz, DMSO) δ |
| | | | 1H-1,3-benzodiazol- | 95.5%; | 8.64 (d, J = 1.8 Hz, 1H), 8.45 (d, |
| | | | 6-yl)quinoxalin-6- | m/z = 401.3 | J = 1.9 Hz, 1H), 8.23 (s, 1H), |
| | | | yl]amino}piperidin-1- | [M + H]⁺ | 7.75 (s, 1H), 7.70 (d, J = 8.3 Hz, |
| | | | yl)ethan-1-one | | 1H), 7.43 (dd, J = 8.4, 1.5 Hz, |
| | | | | | 1H), 7.37 (d, J = 2.5 Hz, 1H), |
| | | | | | 6.92 (d, J = 2.5 Hz, 1H), 6.64 (d, |
| | | | | | J = 7.8 Hz, 1H), 4.31-4.19 (m, |
| | | | | | 1H), 3.86 (s, J = 17.8 Hz, 2H), |
| | | | | | 3.85-3.67 (m, 1H), |
| | | | | | 3.32-3.21 (m, 1H), 2.95-2.83 (m, |
| | | | | | 1H), 2.11-1.95 (m, 2H), |
| | | | | | 1.46-1.25 (m, 1H). |
| 207 | 191 | 402.52 | 1-(3-{[8-(3-methyl-1- | HPLC | 1H NMR (400 MHz, DMSO) δ |
| | | | benzothiophen-5- | 99.1%; | 8.69 (d, J = 1.2 Hz, 2H), |
| | | | yl)quinoxalin-6- | m/z = 403.1 | 8.52-8.46 (m, 2H), 8.04 (dd, J = 8.3, |
| | | | yl]amino}pyrrolidin- | [M + H]⁺ | 0.7 Hz, 2H), 7.94 (d, J = 1.4 Hz, |
| | | | 1-yl)ethan-1-one | | 2H), 7.65-7.59 (m, 2H), |
| | | | | | 7.46 (s, 2H), 7.42 (dd, J = 3.8, 2.7 Hz, |
| | | | | | 2H), 7.00-6.88 (m, 4H), |
| | | | | | 4.36-4.16 (m, 2H), 3.90 (dd, J = 10.5, |
| | | | | | 5.9 Hz, 1H), |
| | | | | | 3.71-3.56 (m, 3H), 3.50-3.37 (m, 5H), |
| | | | | | 2.43 (s, 6H), 2.36-2.16 (m, |
| | | | | | 2H), 2.09-1.89 (m, 8H). |
| | | | | | Mixture of two |
| | | | | | diastereoisomeric conformers |
| 208 | 192 | 386.46 | 1-[(3S)-3-{[8-(1- | HPLC | 1H NMR (400 MHz, DMSO-d6) |
| | | | methyl-1H-indazol-6- | 94.5%; | δ 8.69 (dd, J = 2.0, 0.8 Hz, 1H), |
| | | | yl)quinoxalin-6- | m/z = 387.2 | 8.49 (t, J = 1.7 Hz, 1H), 8.10 (d, |
| | | | yl]amino}pyrrolidin- | [M + H]⁺ | J = 0.9 Hz, 1H), 7.81 (dd, J = 8.0, |
| | | | 1-yl]ethan-1-one | | 1.1 Hz, 2H), 7.41 (dd, J = 4.3, |
| | | | | | 2.6 Hz, 1H), 7.36 (ddd, J = 8.2, |
| | | | | | 2.6, 1.4 Hz, 1H), 6.94 (ddd, |
| | | | | | J = 13.2, 8.8, 4.6 Hz, 2H), |
| | | | | | 4.36-4.17 (m, 1H), 4.08 (s, 3H), |
| | | | | | 3.89 (dd, J = 10.6, 5.8 Hz, 0.5H), |
| | | | | | 3.70-3.55 (m, 1.5H), |
| | | | | | 3.53-3.36 (m, 2H), 2.32-2.16 (m, |
| | | | | | 1H), 2.10-1.88 (m, 4H). |
| | | | | | Mixture of two conformers in |
| | | | | | 1:1 ratio |
| 209 | 193 | 372.43 | 1-(3-{[8-(1-methyl- | HPLC | ¹H NMR (400 MHz, DMSO-d₆) δ |
| | | | 1H-indazol-6- | 97.7%; | 8.70 (d, J = 1.9 Hz, 1H), 8.52 (d, |
| | | | yl)quinoxalin-6- | m/z = 373.1 | J = 1.9 Hz, 1H), 8.10 (d, J = 0.9 Hz, |
| | | | yl]amino}azetidin-1- | [M + H]⁺ | 1H), 7.83-7.78 (m, 2H), |
| | | | yl)ethan-1-one | | 7.38-7.36 (m, 1H), |
| | | | | | 7.35-7.32 (m, 1H), 6.76 (d, J = 2.6 Hz, |
| | | | | | 1H), 4.59 (t, J = 7.8 Hz, 1H), |
| | | | | | 4.42 (dd, J = 12.4, 6.2 Hz, 1H), |
| | | | | | 4.34-4.26 (m, 1H), 4.07 (s, |
| | | | | | 3H), 3.97 (dd, J = 8.6, 4.7 Hz, |
| | | | | | 1H), 3.76 (dd, J = 9.7, 4.8 Hz, |
| | | | | | 1H), 1.80 (s, 3H). |
| 210 | 194 | 400.49 | 1-[(3S)-3-{[8-(1- | HPLC | 1H NMR (400 MHz, DMSO-d6) |
| | | | methyl-1H-indazol-6- | 99.6%; | δ 8.67 (t, J = 2.2 Hz, 1H), |
| | | | yl)quinoxalin-6- | m/z = 401.2 | 8.48 (dd, J = 3.0, 1.9 Hz, 1H), 8.10 (t, |
| | | | yl]amino}piperidin-1- | [M + H]⁺ | J = 1.1 Hz, 1H), 7.85-7.76 (m, |
| | | | yl]ethan-1-one | | 2H), 7.50 (d, J = 2.6 Hz, 0.5H), |
| | | | | | 7.43 (d, J = 2.6 Hz, 0.5H), |
| | | | | | 7.36 (ddd, J = 8.5, 5.8, 1.3 Hz, 1H), |
| | | | | | 6.98 (dd, J = 9.4, 2.6 Hz, 1H), |
| | | | | | 6.71 (d, J = 7.8 Hz, 0.5H), |
| | | | | | 6.67 (d, J = 7.5 Hz, 0.5H), |
| | | | | | 4.59-4.48 (m, 0.5H), 4.08 (d, J = 2.4 Hz, |
| | | | | | 3H), 3.86-3.67 (m, 2H), |
| | | | | | 3.50-3.37 (m, 0.5H), |
| | | | | | 3.27-3.08 (m, 1.5H), 2.59 (dd, J = 12.5, |
| | | | | | 9.5 Hz, 0.5H), |
| | | | | | 2.13-1.95 (m, 4H), 1.86-1.70 (m, 1H), |
| | | | | | 1.70-1.41 (m, 2H). |
| | | | | | Mixture of two conformers |
| 211 | 195 | 400.49 | 1-(4-{[8-(1-methyl- | HPLC | 1H NMR (400 MHz, DMSO) δ |
| | | | 1H-indazol-6- | 99.6%; | 8.65 (d, J = 1.8 Hz, 1H), 8.45 (d, |
| | | | yl)quinoxalin-6- | m/z = 401.4 | J = 1.9 Hz, 1H), 8.09 (d, J = 0.5 Hz, |
| | | | yl]amino}piperidin-1- | [M + H]⁺ | 1H), 7.83-7.76 (m, 2H), |
| | | | yl)ethan-1-one | | 7.40 (d, J = 2.5 Hz, 1H), |
| | | | | | 7.35 (dd, J = 8.6, 0.9 Hz, 1H), |
| | | | | | 6.95 (d, J = 2.4 Hz, 1H), 6.67 (d, J = 7.8 Hz, |
| | | | | | 1H), 4.32-4.21 (m, |
| | | | | | 1H), 4.07 (s, 3H), |
| | | | | | 3.87-3.69 (m, 2H), 3.32-3.21 (m, 1H), |
| | | | | | 2.96-2.84 (m, 1H), |
| | | | | | 2.12-1.94 (m, 5H), 1.41 (td, J = 13.6, 3.6 Hz, |
| | | | | | 1H), 1.30 (td, J = 14.4, 4.1 Hz, |
| | | | | | 1H). |
| 212 | 196 | 400.49 | 1-(4-{[8-(2-methyl- | HPLC | 1H NMR (400 MHz, DMSO) δ |
| | | | 2H-indazol-5- | 99.0%; | 8.64 (d, J = 1.9 Hz, 1H), 8.45 (d, |
| | | | yl)quinoxalin-6- | m/z = 401.3 | J = 1.9 Hz, 1H), 8.40 (s, 1H), |
| | | | yl]amino}piperidin-1- | [M + H]⁺ | 7.88 (s, 1H), 7.63 (d, J = 8.9 Hz, |
| | | | yl)ethan-1-one | | 1H), 7.47 (dd, J = 8.9, 1.6 Hz, |
| | | | | | 1H), 7.36 (d, J = 2.5 Hz, 1H), |
| | | | | | 6.90 (d, J = 2.5 Hz, 1H), 6.61 (d, |
| | | | | | J = 7.8 Hz, 1H), 4.31-4.16 (m, |
| | | | | | 4H), 3.88-3.68 (m, 2H), |
| | | | | | 3.32-3.21 (m, 1H), 2.96-2.85 (m, |
| | | | | | 1H), 2.11-1.95 (m, 5H), |
| | | | | | 1.41 (td, J = 13.9, 4.1 Hz, 1H), |
| | | | | | 1.29 (td, J = 14.2, 4.1 Hz, 1H). |
| 213 | 197 | 372.43 | 1-[(3S)-3-{[8-(1H-1,3- | HPLC | Not determined |
| | | | benzodiazol-2- | 100%; | |
| | | | yl)quinoxalin-6- | m/z = 373.1 | |
| | | | yl]amino}pyrrolidin- | [M + H]⁺ | |
| | | | 1-yl]ethan-1-one | | |
| 214 | 198 | 399.50 | 1-(4-{[8-(1-methyl- | HPLC | 1H NMR (400 MHz, DMSO-d6) |
| | | | 1H-indol-2- | 99.7%; | δ 7.60 (dt, J = 7.8, 0.9 Hz, 1H), |
| | | | yl)quinoxalin-6- | m/z = 400.2 | 7.50 (dd, J = 8.2, 1.0 Hz, 1H), |
| | | | yl]amino}piperidin-1- | [M + H]⁺ | 7.41 (d, J = 2.6 Hz, 1H), |
| | | | yl)ethan-1-one | | 7.21 (ddd, J = 8.2, 7.1, 1.2 Hz, 1H), |
| | | | | | 7.09 (ddd, J = 7.9, 7.1, 1.0 Hz, |
| | | | | | 1H), 7.02 (d, J = 2.6 Hz, 1H), |
| | | | | | 6.74 (d, J = 7.7 Hz, 1H), 6.56 (d, |
| | | | | | J = 0.7 Hz, 1H), 4.29 (d, J = 13.5 Hz, |
| | | | | | 1H), 3.84 (d, J = 14.0 Hz, |
| | | | | | 1H), 3.77 (d, J = 9.8 Hz, 1H), |
| | | | | | 3.48 (s, 3H), 2.90 (t, J = 11.3 Hz, |
| | | | | | 1H), 2.04 (s, 5H), |
| | | | | | 1.53-1.24 (m, 3H). |
| 215 | 199 | 382.47 | 1-(3-{[8-(naphthalen- | HPLC | 1H NMR (400 MHz, DMSO) ? |
| | | | 2-yl)quinoxalin-6- | 99.4%; | 8.69 (d, J = 1.1 Hz, 2H), |
| | | | yl]amino}pyrrolidin- | m/z = 383.2 | 8.54-8.47 (m, 2H), 8.12 (s, 2H), |
| | | | 1-yl)ethan-1-one | [M + H]⁺ | 8.02-7.93 (m, 6H), 7.81-7.73 (m, |
| | | | | | 2H), 7.61-7.52 (m, 4H), |
| | | | | | 7.45 (dd, J = 4.1, 2.6 Hz, 2H), |
| | | | | | 7.01-6.89 (m, 4H), 4.35-4.26 (m, |
| | | | | | 1H), 4.27-4.18 (m, 1H), |
| | | | | | 3.89 (dd, J = 10.6, 5.9 Hz, 1H), |
| | | | | | 3.68-3.56 (m, 3H), 3.51-3.37 (m, |
| | | | | | 4H), 2.37-2.16 (m, 2H), |
| | | | | | 2.07-1.88 (m, 8H). Mixture of two |
| | | | | | diastereomeric conformers |
| 216 | 200 | 428.50 | 6-[(1- | HPLC | 1H NMR (400 MHz, DMSO) δ |
| | | | acetylpyrrolidin-3- | 99.4%; | 9.20 (d, J = 1.6 Hz, 2H), 7.79 (s, |
| | | | yl)amino]-8-(1- | m/z = 429.2 | 2H), 7.73-7.62 (m, 4H), |
| | | | methyl-1H-indol-6- | [M + H]⁺ | 7.52 (t, J = 2.9 Hz, 2H), |
| | | | yl)quinoxaline-2- | | 7.44-7.33 (m, 4H), 7.30 (dd, J = 12.7, 6.5 Hz, |
| | | | carboxamide | | 2H), 7.23 (s, 2H), 6.95 (dd, J = 13.5, |
| | | | | | 2.4 Hz, 2H), 6, 49 (d, J = 3.0 Hz, |
| | | | | | 2H), 4.31 (ddd, J = 32.4, |
| | | | | | 10.0, 5.0 Hz, 2H), 3.91 (dd, J = 10.6, |
| | | | | | 5.8 Hz, 1H), 3.84 (s, 6H), |
| | | | | | 3.71-3.59 (m, 3H), |
| | | | | | 3.52-3.39 (m, 5H), 2.38-2.16 (m, |
| | | | | | 2H), 2.11-1.92 (m, 8H). |
| | | | | | Mixture of two |
| | | | | | diastereoisomeric conformers |
| | | | | | of 1-acetylpyrolidine |
| 217 | 201 | 410.48 | 6-[(1- | HPLC | 1H NMR (400 MHz, DMSO) δ |
| | | | acetylpyrrolidin-3- | 88.8%; | 9.02 (d, J = 1.0 Hz, 1H), |
| | | | yl)amino]-8-(1- | m/z = 411.2 | 7.68-7.59 (m, 3H), 7.53-7.49 (m, |
| | | | methyl-1H-indol-6- | [M + H]⁺ | 1H), 7.43 (d, J = 2.5 Hz, 1H), |
| | | | yl)quinoxaline-2- | compound | 7.30-7.23 (m, 1H), 6.94 (dd, J = 14.2, |
| | | | carbonitrile | contains | 2.5 Hz, 1H), 6.50 (d, J = 3.0 Hz, |
| | | | | 9.5% of | 1H), 4.42-4.26 (m, |
| | | | | 3- | 1H), 3.90 (dd, J = 10.7, 5.9 Hz, |
| | | | | carbonitrile | 0.5H), 3.83 (s, 3H), |
| | | | | derivative. | 3.69-3.58 (m, 1.5H), 3.51-3.39 (m, 2H), |
| | | | | | 2.34-2.19 (m, 1H), |
| | | | | | 2.07-1.91 (m, 4H). Mixture of |
| | | | | | diastereoisomeric conformers |
| 218 | 202 | 400.87 | N-[(2- | HPLC | 1H NMR (400 MHz, DMSO) δ |
| | | | chloropyrimidin-5- | 94.1%; | 8.87 (s, 2H), 8.64 (d, J = 1.9 Hz, |
| | | | yl)methyl]-8-(1- | m/z = 401.5 | 1H), 8.49 (d, J = 1.9 Hz, 1H), |
| | | | methyl-1H-indol-5- | [M + H]⁺ | 7.77 (d, J = 1.1 Hz, 1H), 7.51 (d, |
| | | | yl)quinoxalin-6- | | J = 8.5 Hz, 1H), 7.42-7.34 (m, |
| | | | amine | | 3H), 7.26 (t, J = 5.9 Hz, 1H), |
| | | | | | 6.83 (d, J = 2.6 Hz, 1H), 6.49 (d, |
| | | | | | J = 3.1 Hz, 1H), 4.57 (d, J = 5.8 Hz, |
| | | | | | 2H), 3.84 (s, 3H). |
| 219 | 203 | 520.61 | N-(4- | HPLC | 1H NMR (400 MHz, DMSO) d |
| | | | methanesulfonylpyridin- | 96.9%; | 8.92 (d, J = 1.7 Hz, 1H), 8.89 (d, |
| | | | 2-yl)-8-(1- | m/z = 521.3 | J = 1.7 Hz, 1H), 8.62 (s, 1H), |
| | | | methyl-1H-indol-6- | [M + H] | 8.56 (d, J = 5.2 Hz, 1H), 8.42 (d, |
| | | | yl)-N-[(pyridin-3- | | J = 3.5 Hz, 1H), 8.03 (d, J = 2.5 Hz, |
| | | | yl)methyl]quinoxalin- | | 1H), 7.95 (d, J = 2.5 Hz, 1H), |
| | | | 6-amine | | 7.82 (d, J = 7.9 Hz, 1H), 7.65 (s, |
| | | | | | 1H), 7.61 (d, J = 8.2 Hz, 1H), |
| | | | | | 7.47 (s, 1H), 7.40 (d, J = 3.0 Hz, |
| | | | | | 1H), 7.36-7.34 (m, 1H), |
| | | | | | 7.34-7.30 (m, 2H), 6.48 (d, J = 3.0 Hz, |
| | | | | | 1H), 5.61 (s, 2H), 3.82 (s, |
| | | | | | 3H), 3.28 (s, 3H). |
| 220 | 204 | 520.61 | N-(4- | HPLC | 1H NMR (400 MHz, DMSO) d |
| | | | methanesulfonylpyridin- | 95.5%; | 8.95 (d, J = 5.1 Hz, 1H), 8.81 (s, |
| | | | 3-yl)-8-(1- | m/z = 521.3 | 1H), 8.75 (d, J = 1.8 Hz, 1H), |
| | | | methyl-1H-indol-6- | [M + H] | 8.70 (d, J = 1.7 Hz, 1H), 8.66 (d, |
| | | | yl)-N-[(pyridin-3- | | J = 1.8 Hz, 1H), 8.51 (dd, J = 4.7, |
| | | | yl)methyl | | 1.2 Hz, 1H), 8.10 (d, J = 5.1 Hz, |
| | | | | | 1H), 7.96-7.90 (m, 1H), |
| | | | | | 7.56 (d, J = 8.2 Hz, 1H), 7.45 (s, |
| | | | | | 1H), 7.42-7.36 (m, 2H), |
| | | | | | 7.28 (d, J = 2.8 Hz, 1H), 7.18 (dd, J = 8.2, |
| | | | | | 1.4 Hz, 1H), 7.10 (d, J = 2.8 Hz, |
| | | | | | 1H), 6.45 (d, J = 3.0 Hz, 1H), |
| | | | | | 5.24 (s, 2H), 3.77 (s, 3H), |
| | | | | | 3.36 (s, 3H). |
| 221 | 205 | 442.526 | 8-(1-methyl-1H- | HPLC | 1H NMR (400 MHz, DMSO) d |
| | | | indol-6-yl)-N- | 96.8%; | 8.76 (d, J = 1.8 Hz, 1H), 8.72 (d, |
| | | | (pyridin-2-yl)-N- | m/z = 443.4 | J = 2.6 Hz, 1H), 8.67 (d, J = 1.8 Hz, |
| | | | [(pyridin-3- | [M + H] | 1H), 8.54 (dd, J = 4.5, 1.6 Hz, |
| | | | yl)methyl]quinoxalin- | | 2H), 8.38 (dd, J = 4.7, 1.3 Hz, |
| | | | 6-amine | | 1H), 7.90 (ddd, J = 8.3, 2.7, |
| | | | | | 1.4 Hz, 1H), 7.58 (d, J = 8.3 Hz, |
| | | | | | 1H), 7.55 (s, 1H), 7.50 (d, J = 2.8 Hz, |
| | | | | | 1H), 7.49-7.43 (m, |
| | | | | | 3H), 7.38 (d, J = 3.1 Hz, 1H), |
| | | | | | 7.31 (d, J = 2.8 Hz, 1H), |
| | | | | | 7.21 (dd, J = 8.1, 1.4 Hz, 1H), |
| | | | | | 6.46 (d, J = 3.1 Hz, 1H), 5.37 (s, 2H), |
| | | | | | 3.78 (s, 3H). |
| 222 | 206 | 442.526 | 8-(1-methyl-1H- | HPLC | 1H NMR (400 MHz, DMSO) d |
| | | | indol-6-yl)-N- | 98.296; | 8.86 (d, J = 1.8 Hz, 1H), 8.81 (d, |
| | | | (pyridin-2-yl)-N- | m/z = 443.4 | J = 1.8 Hz, 1H), 8.62 (d, J = 1.8 Hz, |
| | | | [(pyridin-3- | [M + H] | 1H), 8.42 (dd, J = 4.8, 1.6 Hz, |
| | | | yl)methyl]quinoxalin- | | 1H), 8.31 (dd, J = 4.9, 1.2 Hz, |
| | | | 6-amine | | 1H), 7.84-7.78 (m, J = 10.8, |
| | | | | | 2.5 Hz, 3H), |
| | | | | | 7.68-7.62 (m, 2H), 7.61 (d, J = 8.2 Hz, |
| | | | | | 1H), 7.40 (d, J = 3.1 Hz, 1H), |
| | | | | | 7.33 (ddd, J = 7.9, 4.8, 0.5 Hz, |
| | | | | | 1H), 7.29 (dd, J = 8.2, 1.4 Hz, |
| | | | | | 1H), 7.15 (d, J = 8.5 Hz, 1H), |
| | | | | | 6.93 (dd, J = 6.8, 5.3 Hz, 1H), |
| | | | | | 6.47 (dd, J = 3.0, 0.6 Hz, 1H), |
| | | | | | 5.55 (s, 2H), 3.82 (s, 3H). |
| 223 | 207 | 446.518 | N-(1-methyl-1H- | HPLC | 1H NMR (400 MHz, DMSO) d |
| | | | 1,2,3-triazol-5-yl)-8- | 97.2%; | 8.80 (d, J = 1.8 Hz, 1H), 8.70 (d, |
| | | | (1-methyl-1H-indol- | m/z = 447.4 | J = 1.8 Hz, 1H), 8.68 (d, J = 1.7 Hz, |
| | | | 6-yl)-N-[(pyridin-3- | [M + H] | 1H), 8.51 (dd, J = 4.6, 1.3 Hz, |
| | | | yl)methyl]quinoxalin- | | 1H), 7.92 (s, 1H), |
| | | | 6-amine | | 7.89-7.85 (m, 1H), 7.59 (d, J = 8.2 Hz, |
| | | | | | 1H), 7.55 (s, 1H), |
| | | | | | 7.42-7.38 (m, 2H), 7.26 (d, J = 2.8 Hz, |
| | | | | | 1H), 7.21 (dd, J = 8.2, 1.4 Hz, |
| | | | | | 1H), 7.13 (d, J = 2.8 Hz, 1H), |
| | | | | | 6.47 (d, J = 3.0 Hz, 1H), 5.22 (s, |
| | | | | | 2H), 3.84 (s, 3H), 3.80 (s, 3H). |
| 224 | 208 | 490.611 | 1-[3-({[8-(1-methyl- | HPLC | 1H NMR (400 MHz, DMSO) d |
| | | | 1H-indol-6- | 97.8%; | 8.79 (dd, J = 4.1, 1.7 Hz, 1H), |
| | | | yl)quinoxalin-6- | m/z = 491.2 | 8.67 (dd, J = 3.3, 1.9 Hz, 1H), |
| | | | yl](pyridin-3- | [M + H] | 8.62 (m, 1H), 8.38 (dd, J = 4.5, |
| | | | yl)amino}methyl)piperidin- | | 0.9 Hz, 1H), 7.85-7.76 (m, |
| | | | 1-yl]ethan-1- | | 1H), 7.60 (t, J = 8.9 Hz, 2H), |
| | | | one | | 7.48-7.33 (m, 4H), |
| | | | | | 7.25-7.19 (m, 1H), 6.46 (d, J = 3.0 Hz, |
| | | | | | 1H), 4.45 (d, J = 10.7 Hz, |
| | | | | | 1H), 4.16 (d, J = 12.4 Hz, 1H), |
| | | | | | 4.02-3.83 (m, 2H), 3.80 (s, |
| | | | | | 3H), 3.70 (d, J = 12.9 Hz, 1H), |
| | | | | | 2.98 (t, J = 11.7 Hz, 1H), 2.60 (t, |
| | | | | | J = 11.9 Hz, 1H), |
| | | | | | 2.01-1.77 (m, 6H), 1.71-1.58 (m, 1H). |
| 225 | 209 | 520.61 | N-(5- | HPLC | 1H NMR (400 MHz, DMSO)d |
| | | | methanesulfonylpyridin- | 95.5%; | 8.89 (d, J = 2.6 Hz, 1H), 8.84 (d, |
| | | | 3-yl)-8-(1- | m/z = 521.3 | J = 1.8 Hz, 1H), 8.78 (d, J = 1.8 Hz, |
| | | | methyl-1H-indol-6- | [M + H] | 1H), 8.73-8.67 (m, J = 2.2 Hz, |
| | | | yl)-N-[(pyridin-3- | | 2H), 8.47 (dd, J = 4.8, 1.4 Hz, |
| | | | yl)methyl]quinoxalin- | | 1H), 8.24-8.20 (m, 1H), |
| | | | 6-amine | | 7.89 (d, J = 7.9 Hz, 1H), 7.76 (d, |
| | | | | | J = 2.7 Hz, 1H), 7.71 (d, J = 2.7 Hz, |
| | | | | | 1H), 7.63-7.58 (m, J = 7.8 Hz, |
| | | | | | 2H), 7.42-7.35 (m, J = 7.9, |
| | | | | | 4.0 Hz, 2H), 7.28 (dd, J = 8.3, |
| | | | | | 1.3 Hz, 1H), 6.47 (d, J = 3.1 Hz, |
| | | | | | 1H), 5.50 (s, 2H), 3.80 (s, 3H), |
| | | | | | 3.34 (s, 3H). |
| 226 | 210 | 520.61 | N-(2- | HPLC | 1H NMR (400 MHz, DMSO) d |
| | | | methanesulfonylpyridin- | 96.0%; | 8.97 (d, J = 1.8 Hz, 1H), 8.95 (d, |
| | | | 4-yl)-8-(1- | m/z = 521.3 | J = 1.8 Hz, 1H), 8.64 (d, J = 1.8 Hz, |
| | | | methyl-1H-indol-6- | [M + H] | 1H), 8.48 (dd, J = 4.8, 1.4 Hz, |
| | | | yl)-N-[(pyridin-3- | | 1H), 8.42 (d, J = 5.8 Hz, 1H), |
| | | | yl)methyl]quinoxalin- | | 8.10 (d, J = 2.5 Hz, 1H), 7.97 (d, |
| | | | 6-amine | | J = 2.5 Hz, 1H), 7.84 (dt, J = 7.7, |
| | | | | | 1.8 Hz, 1H), 7.70 (s, 1H), |
| | | | | | 7.63 (d, J = 8.2 Hz, 1H), 7.52 (d, J = 2.5 Hz, |
| | | | | | 1H), 7.41 (d, J = 3.1 Hz, |
| | | | | | 1H), 7.37 (dt, J = 8.3, 2.9 Hz, |
| | | | | | 2H), 7.22 (dd, J = 5.9, 2.6 Hz, |
| | | | | | 1H), 6.49 (d, J = 3.0 Hz, 1H), |
| | | | | | 5.46 (s, 2H), 3.83 (s, 3H), |
| | | | | | 3.23 (s, 3H). |
| 227 | 211 | 485.551 | 3-{[8-(1-methyl-1H- | HPLC | 1H NMR (400 MHz, DMSO) d |
| | | | indol-6-yl)quinoxalin- | 92.4%; | 8.72 (d, J = 1.7 Hz, 1H), 8.70 (d, |
| | | | 6-yl][(pyridin-3- | m/z = 486.3 | J = 1.8 Hz, 1H), 8.66 (s, 1H), |
| | | | yl)methyl]amino}pyridine- | [M + H] | 8.62 (d, J = 4.9 Hz, 1H), 8.60 (d, |
| | | | 4-carboxamide | | J = 1.8 Hz, 1H), 8.48 (dd, J = 4.7, |
| | | | | | 1.5 Hz, 1H), 8.10 (s, 1H), |
| | | | | | 7.94 (ddd, J = 8.1, 1.8 Hz, 1H), |
| | | | | | 7.70 (s, 1H), 7.56 (d, J = 4.9 Hz, |
| | | | | | 1H), 7.54 (d, J = 8.2 Hz, 1H), |
| | | | | | 7.47-7.44 (m, 1H), |
| | | | | | 7.37 (d + ddd, J = 8.2, 4.8, 3.1, 0.6 Hz, |
| | | | | | 2H), 7.21 (d, J = 2.9 Hz, 1H), |
| | | | | | 7.16 (dd, J = 8.2, 1.5 Hz, 1H), |
| | | | | | 7.04 (d, J = 2.8 Hz, 1H), |
| | | | | | 6.44 (dd, J = 3.1, 0.8 Hz, 1H), |
| | | | | | 5.17 (s, 2H), 3.76 (s, 3H). |
| 228 | 212 | 448.57 | 8-(1-methyl-1H- | HPLC | not determined |
| | | | indol-6-yl)-N- | 96.2%; | |
| | | | [piperidin-4- | m/z = 449.6 | |
| | | | yl(pyridin-4- | [M + H] | |
| | | | yl)methyl]quinoxalin- | | |
| | | | 6-amine | | |
| 229 | 213 | 449.56 | 8-(1-methyl-1H- | HPLC | not determined |
| | | | indol-6-yl)-N- | 100%; | |
| | | | [piperidin-4- | m/z = 494.7 | |
| | | | yl(pyridazin-3- | [M − H] | |
| | | | yl)methyl]quinoxalin- | | |
| | | | 6-amine | | |
| 230 | 214 | 380.46 | N-[(4-aminopyridin- | HPLC 98%; | 1H NMR (400 MHz; DMSO) δ |
| | | | 3-yl)methyl]-8-(1- | m/z = 381.1 | 8.80 (d, J = 1.8 Hz, 1H), 8.70 (d, |
| | | | methyl-1H- | [M + H] | J = 2.5 Hz, 1H), 8.67 (d, J = 1.8 Hz, |
| | | | indol-6-yl)quinoxalin- | | 1H), 8.23 (dd, J = 5.0, 1.8 Hz, |
| | | | 6-amine | | 1H), 7.89 (d, J = 2.5 Hz, 1H), |
| | | | | | 7.71 (s, 1H), 7.64 (d, J = 8.1 Hz, |
| | | | | | 1H), 7.59 (dd, J = 7.3, 1.7 Hz, |
| | | | | | 1H), 7.40 (d, J = 3.1 Hz, 1H), |
| | | | | | 7.32 (dd, J = 8.2, 1.4 Hz, 1H), |
| | | | | | 6.90 (dd, J = 7.2, 5.0 Hz, 1H), |
| | | | | | 6.49 (dd, J = 3.1, 0.7 Hz, 1H), |
| | | | | | 3.93 (s, 2H), 3.84 (s, 3H) |
| 231 | 215 | 395.47 | N-[(4- | HPLC 99%; | 1H NMR (400 MHz, DMSO) δ |
| | | | methoxypyridin-3- | m/z = 396.4 | 8.62 (d, J = 1.9 Hz, 1H), 8.46 (d, |
| | | | yl)methyl]-8-(1- | [M + H] | J = 1.9 Hz, 1H), 8.40 (d, J = 4.6 Hz, |
| | | | methyl-1H- | | 2H), 7.62 (s, 1H), 7.60 (d, J = 8.2 Hz, |
| | | | indol-6-yl)quinoxalin- | | 1H), 7.44 (d, J = 2.6 Hz, |
| | | | 6-amine | | 1H), 7.38 (d, J = 3.0 Hz, 1H), |
| | | | | | 7.26 (dd, J = 8.1, 1.4 Hz, 1H), |
| | | | | | 7.11 (d, J = 5.8 Hz, 2H), 6.77 (d, |
| | | | | | J = 2.5 Hz, 1H), 6.47 (d, J = 3.0 Hz, |
| | | | | | 1H), 4.44 (d, J = 5.7 Hz, 2H), |
| | | | | | 3.94 (s, 3H), 3.81 (s, 3H). |
| 232 | 216 | 491.60 | 1-{4-[3-({[8-(1- | HPLC 94%; | 1H NMR (400 MHz, DMSO) δ |
| | | | methyl-1H-indol-6- | m/z = 492.3 | 8.62 (d, J = 1.9 Hz, 1H), 8.51 (s, |
| | | | yl)quinoxalin-6- | [M + H] | 1H), 8.46 (d, J = 1.9 Hz, 1H), |
| | | | yl]amino}methyl)pyridin- | | 8.33 (d, J = 5.5 Hz, 1H), |
| | | | 4-yl]piperazin-1- | | 7.63-7.58 (m, 2H), 7.42 (d, J = 2.5 Hz, |
| | | | yl}ethan-1- | | 1H), 7.39 (d, J = 3.0 Hz, 1H), |
| | | | one | | 7.33 (d, J = 5.8 Hz, 1H), 7.24 (d, |
| | | | | | J = 8.1 Hz, 1H), 7.02 (d, J = 5.4 Hz, |
| | | | | | 1H), 6.79 (d, J = 2.3 Hz, 1H), |
| | | | | | 6.47 (d, J = 3.0 Hz, 1H), 4.48 (d, |
| | | | | | J = 5.5 Hz, 2H), 3.81 (s, 3H), |
| | | | | | 3.65 (s, 4H), 3.04 (d, J = 28.3 Hz, |
| | | | | | 4H), 2.05 (s, 3H). |
| 233 | 217 | 491.60 | 1-[4-({[8-(3-methyl- | HPLC 95%; | 1H NMR (400 MHz, DMSO) δ |
| | | | 1-benzofuran-5- | m/z = 492.3 | 8.71 (d, J = 1.8 Hz, 1H), 8.57 (s, |
| | | | yl)quinoxalin-6- | [M + H] | 1H), 8.44 (dd, J = 8.5, 1.6 Hz, |
| | | | yl]amino}(pyridin-3- | | 2H), 7.85 (d, J = 7.8 Hz, 1H), |
| | | | yl)methyl)piperidin- | | 7.82 (d, J = 1.1 Hz, 1H), 7.74 (s, |
| | | | 1-yl]ethan-1- | | 1H), 7.60 (d, J = 8.5 Hz, 1H), |
| | | | one | | 7.52 (dd, J = 6.0, 2.3 Hz, 2H), |
| | | | | | 7.37 (dd, J = 7.7, 4.8 Hz, 1H), |
| | | | | | 7.26 (d, J = 7.9 Hz, 1H), 6.70 (s, |
| | | | | | 1H), 4.49 (ddd, J = 47.3, 24.3, |
| | | | | | 10.9 Hz, 3H), 3.85 (dd, J = 30.5, |
| | | | | | 15.7 Hz, 1H), 2.97 (dd, J = 28.1, |
| | | | | | 13.8 Hz, 2H), 2.43 (d, J = 14.0 Hz, |
| | | | | | 1H), 2.24 (d, J = 1.0 Hz, 3H), |
| | | | | | 2.03 (d, J = 8.1 Hz, 1H), 1.98 (d, |
| | | | | | J = 1.8 Hz, 3H), 1.92 (d, J = 14.2 Hz, |
| | | | | | 1H). |
| 234 | 218 | 451.578 | N-[(1-methyl-1H- | HPLC 70%; | 1H NMR (400 MHz, DMSO) δ |
| | | | imidazol-4- | m/z = 452.3 | 8.57 (d, J = 1.8 Hz, 1H), 8.39 (d, |
| | | | yl)(piperidin-4- | [M + H] | J = 1.9 Hz, 1H), 7.61-7.57 (m, |
| | | | yl)methyl]-8-(1- | | 2H), 7.52 (d, J = 2.5 Hz, 1H), |
| | | | methyl-1H-indol-6- | | 7.49 (s, 1H), 7.38 (d, J = 3.1 Hz, |
| | | | yl)quinoxalin-6- | | 1H), 7.25 (dd, J = 8.3, 1.3 Hz, |
| | | | amine | | 1H), 6.99 (d, J = 1.3 Hz, 1H), |
| | | | | | 6.78 (dd, J = 9.6, 5.2 Hz, 2H), |
| | | | | | 6.47 (d, J = 3.0 Hz, 1H), |
| | | | | | 4.32-4.25 (m, 1H), 3.81 (s, J = 2.7 Hz, |
| | | | | | 3H), 3.58 (s, 3H), 3.51 (s, |
| | | | | | 1H), 3.43-3.38 (m, 1H), |
| | | | | | 3.01-2.91 (m, 2H), 1.98-1.79 (m, |
| | | | | | 4H), 1.44 (d, J = 11.2 Hz, 2H). |
| 235 | 219 | 472.55 | N-[(6- | HPLC | 1H NMR (400 MHz, DMSO) δ |
| | | | methoxypyridin-3- | 95.2%; | 8.74 (d, J = 2.0 Hz, 1H), 8.62 (d, |
| | | | yl)(pyridin-3- | m/z = 473.3 | J = 1.9 Hz, 1H), 8.53-8.46 (m, |
| | | | yl)methyl]-8- | [M + H] | 2H), 8.33 (d, J = 2.4 Hz, 1H), |
| | | | (1-methyl-1H-indol- | | 7.87 (dt, J = 7.9, 1.8 Hz, 1H), |
| | | | 6-yl)quinoxalin-6- | | 7.79 (dd, J = 8.6, 2.5 Hz, 1H), |
| | | | amine | | 7.63-7.57 (m, 3H), 7.52 (d, J = 7.0 Hz, |
| | | | | | 1H), 7.44-7.37 (m, |
| | | | | | 2H), 7.28 (dd, J = 8.1, 1.4 Hz, |
| | | | | | 1H), 6.86 (d, J = 8.6 Hz, 1H), |
| | | | | | 6.83 (d, J = 2.5 Hz, 1H), 6.47 (d, |
| | | | | | J = 3.0 Hz, 1H), 6.09 (d, J = 7.0 Hz, |
| | | | | | 1H), 3.82 (d, J = 8.2 Hz, 6H). |
| 236 | 220 | 381.44 | 3-({[8-(1-methyl-1H- | HPLC | 1H NMR (400 MHz, DMSO) δ |
| | | | indol-6-yl)quinoxalin- | 89.1%; | 8.63 (d, J = 1.8 Hz, 1H), 8.48 (d, |
| | | | 6- | m/z = 382.2 | J = 1.9 Hz, 1H), 8.31 (s, 1H), |
| | | | yl]amino}methyl)- | [M + H] | 8.12 (td, J = 3.9, 1.9 Hz, 1H), |
| | | | 1λ⁵-pyridin-1-one | | 7.65-7.58 (m, 2H), 7.42 (m, |
| | | | | | 3H), 7.39 (d, J = 3.0 Hz, 1H), |
| | | | | | 7.34 (t, J = 6.2 Hz, 1H), |
| | | | | | 7.27 (dd, J = 8.2, 1.4 Hz, 1H), |
| | | | | | 6.80 (d, J = 2.5 Hz, 1H), 6.48 (d, J = 3.0 Hz, |
| | | | | | 1H), 4.52 (d, J = 6.1 Hz, |
| | | | | | 2H), 3.82 (s, 3H). |
| 237 | 221 | 518.67 | 2-methyl-1-[4-({[8- | HPLC | 1H NMR (400 MHz, DMSO) δ |
| | | | (1-methyl-1H-indol- | 97.9%; | 8.72 (s, 1H), 8.57 (d, J = 1.9 Hz, |
| | | | 6- | m/z = 519.3 | 1H), 8.45 (d, J = 3.4 Hz, 1H), |
| | | | yl)quinoxalin-6- | [M + H] | 8.42 (d, J = 1.9 Hz, 1H), 7.87 (d, J = 7.9 Hz, |
| | | | yl]amino}(pyridin-3- | | 1H), 7.60 (d, J = 7.7 Hz, |
| | | | yl)methyl)piperidin- | | 2H), 7.50 (d, J = 2.3 Hz, 1H), |
| | | | 1-yl]propan-1-one | | 7.38 (dd, J = 7.0, 3.9 Hz, 2H), |
| | | | | | 7.26 (dd, J = 13.1, 4.8 Hz, 2H), |
| | | | | | 6.71 (d, J = 2.2 Hz, 1H), 6.48 (d, J = 3.1 Hz, |
| | | | | | 1H), 4.61-4.53 (m, |
| | | | | | 1H), 4.48 (d, 1H), 4.39 (d, 1H), |
| | | | | | 4.09-4.00 (m, 1H), |
| | | | | | 4.00-3.91 (m, 1H), 3.82 (s, 3H), |
| | | | | | 3.08-2.93 (m, J = 22.5 Hz, 1H), |
| | | | | | 2.93-2.78 (m, J = 7.8 Hz, 1H), |
| | | | | | 2.12-2.02 (m, 2H), 1.39-1.27 (m, 2H), |
| | | | | | 1.05-0.93 (m, J = 11.6, 6.3 Hz, |
| | | | | | 6H). |
| 238 | 222 | 504.64 | 1-[4-({[8-(1-methyl- | HPLC | 1H NMR (400 MHz, DMSO) δ |
| | | | 1H-indol-6- | 96.2%; | 8.72 (d, J = 1.7 Hz, 1H), 8.57 (d, |
| | | | yl)quinoxalin-6- | m/z = 505.3 | J = 1.9 Hz, 1H), 8.45 (dd, J = 4.7, |
| | | | yl]amino}(pyridin-3- | [M + H] | 1.6 Hz, 1H), 8.42 (d, J = 1.9 Hz, |
| | | | yl)methyl)piperidin- | | 1H), 7.86 (d, J = 7.9 Hz, 1H), |
| | | | 1- | | 7.60 (d, J = 7.8 Hz, 2H), 7.51 (s, |
| | | | yl]propan-1-one | | 1H), 7.38 (dd, J = 7.3, 3.9 Hz, |
| | | | | | 2H), 7.29-7.23 (m, 2H), |
| | | | | | 6.70 (s, 1H), 6.48 (dd, J = 3.1, 0.7 Hz, |
| | | | | | 1H), 4.56 (d, J = 7.6 Hz, 1H), |
| | | | | | 4.48 (d, J = 12.7 Hz, 1H), |
| | | | | | 4.40 (d, J = 11.6 Hz, 1H), |
| | | | | | 3.97-3.84 (m, 2H), 3.82 (s, 3H), 2.95 (dd, |
| | | | | | J = 31.7, 12.5 Hz, 1H), |
| | | | | | 2.35-2.24 (m, 3H), 1.39-1.26 (m, |
| | | | | | 3H), 0.97 (t, J = 7.4, 6.1 Hz, |
| | | | | | 3H). |
| 239 | 223 | 487.61 | 2-[4-({[8-(1-methyl- | HPLC | 1H NMR (400 MHz, DMSO) δ |
| | | | 1H-indol-6- | 95.5%; | 8.72 (d, J = 1.7 Hz, 1H), 8.57 (d, |
| | | | yl)quinoxalin-6- | m/z = 488.3 | J = 1.9 Hz, 1H), 8.44 (dd, J = 4.7, |
| | | | yl]amino}(pyridin-3- | [M + H] | 1.5 Hz, 1H), 8.42 (d, J = 1.9 Hz, |
| | | | yl)methyl)piperidin- | | 1H), 7.88-7.85 (m, 1H), |
| | | | 1- | | 7.62-7.57 (m, 2H), 7.50 (d, J = 2.6 Hz, |
| | | | yl]acetonitrile | | 1H), 7.38 (dd, J = 7.9, |
| | | | | | 3.9 Hz, 2H), 7.25 (dd, J = 8.3, |
| | | | | | 1.3 Hz, 2H), 6.70 (d, J = 2.4 Hz, |
| | | | | | 1H), 6.48 (dd, J = 3.1, 0.7 Hz, |
| | | | | | 1H), 4.51 (t, J = 8.0 Hz, 1H), |
| | | | | | 3.82 (s, 3H), 3.70 (s, 2H), |
| | | | | | 2.83 (dd, J = 39.8, 10.9 Hz, 3H), |
| | | | | | 2.18-2.05 (m, J = 24.2, 9.7 Hz, 4H), |
| | | | | | 1.75 (d, J = 7.6 Hz, 1H), 1.29 (d, |
| | | | | | J = 10.9 Hz, 1H). |
| 240 | 224 | 472.55 | N-[(2- | HPLC | 1H NMR (400 MHz, DMSO) δ |
| | | | methoxypyridin-4- | 91.4%; | 8.77 (d, J = 2.0 Hz, 1H), 8.63 (d, |
| | | | yl)(pyridin-3- | m/z = 473.2 | J = 1.9 Hz, 1H), 8.52 (dd, J = 4.8, |
| | | | yl)methyl]-8- | [M + H] | 1.6 Hz, 1H), 8.50 (d, J = 1.9 Hz, |
| | | | (1-methyl-1H-indol- | | 1H), 8.17 (d, J = 5.4 Hz, 1H), |
| | | | 6-yl)quinoxalin-6- | | 7.88 (dt, J = 7.9, 1.9 Hz, 1H), |
| | | | amine | | 7.64-7.59 (m, 3H), 7.57 (d, J = 7.4 Hz, |
| | | | | | 1H), 7.43 (dd, J = 8.1, |
| | | | | | 5.0 Hz, 1H), 7.39 (d, J = 3.0 Hz, |
| | | | | | 1H), 7.28 (dd, J = 8.2, 1.4 Hz, |
| | | | | | 1H), 7.15 (dd, J = 5.3, 1.3 Hz, |
| | | | | | 1H), 7.00 (s, 1H), 6.83 (d, J = 2.5 Hz, |
| | | | | | 1H), 6.48 (dd, J = 3.0, |
| | | | | | 0.7 Hz, 1H), 6.09 (d, J = 7.2 Hz, |
| | | | | | 1H), 3.83 (s, 3H), 3.82 (s, 3H). |
| 241 | 225 | 459.56 | 8-(1-methyl-1H- | HPLC | 1H NMR (400 MHz, DMSO) δ |
| | | | indol-6-yl)-N-{1-[4- | 97.7%; | 8.74 (s, 1H), 8.53 (d, J = 1.9 Hz, |
| | | | (1-methyl-1H- | m/z = 460.3 | 1H), 8.41 (d, J = 1.9 Hz, 1H), |
| | | | pyrazol-4-yl)pyridin- | [M + H] | 8.38 (d, J = 5.1 Hz, 1H), 8.23 (s, |
| | | | 3- | | 1H), 7.90 (s, 1H), |
| | | | yl]ethyl}quinoxalin- | | 7.61-7.57 (m, 2H), 7.45 (d, J = 6.5 Hz, |
| | | | 6-amine | | 1H), 7.42 (d, J = 5.1 Hz, 1H), |
| | | | | | 7.39 (d, J = 3.1 Hz, 2H), |
| | | | | | 7.21 (dd, J = 8.3, 1.2 Hz, 1H), |
| | | | | | 6.47 (d, J = 3.1 Hz, 1H), 6.20 (s, 1H), |
| | | | | | 4.97-4.90 (m, 1H), 3.97 (s, |
| | | | | | 3H), 3.81 (s, 3H), 1.66 (d, J = 6.7 Hz, |
| | | | | | 3H). |
| 242 | 226 | 477.62 | 8-(1-methyl-1H- | HPLC | 1H NMR (400 MHz, DMSO) δ |
| | | | indol-6-yl)-N-{1-[4- | 97.5%; | 8.59 (d, J = 1.9 Hz, 3H), 8.41 (d, |
| | | | (4- | m/z = 478.3 | J = 1.9 Hz, 1H), 8.25 (d, J = 5.5 Hz, |
| | | | methylpiperazin-1- | [M + H] | 1H), 7.57 (d, J = 8.4 Hz, 1H), |
| | | | yl)pyridin-3- | | 7.38 (d, J = 3.0 Hz, 1H), 7.35 (d, |
| | | | yl]ethyl}quinoxalin- | | J = 2.5 Hz, 1H), 7.30 (d, J = 7.4 Hz, |
| | | | 6- | | 1H), 7.19 (d, J = 9.3 Hz, 1H), |
| | | | amine | | 7.02 (d, J = 5.5 Hz, 1H), 6.88 (d, |
| | | | | | J = 2.4 Hz, 1H), 6.46 (d, J = 3.0 Hz, |
| | | | | | 1H), 4.91-4.83 (m, 1H), |
| | | | | | 3.80 (s, 3H), 3.13-3.06 (m, |
| | | | | | 2H), 2.98 (s, 2H), 2.63 (s, 4H), |
| | | | | | 2.28 (s, 3H), 1.66 (d, J = 6.8 Hz, |
| | | | | | 3H). |
| 243 | 227 | 369.43 | N-[(1-methyl-1H- | HPLC | 1H NMR (400 MHz, DMSO) δ |
| | | | 1,2,3-triazol-5- | 94.2%; | 8.66 (d, J = 1.9 Hz, 1H), 8.50 (d, |
| | | | yl)methyl]-8-(1- | m/z = 370.1 | J = 1.9 Hz, 1H), 7.71 (s, 1H), |
| | | | methyl-1H-indol-6- | [M + H] | 7.65-7.58 (m, 2H), 7.40 (dd, J = 13.6, |
| | | | yl)quinoxalin-6- | | 2.8 Hz, 2H), 7.26 (d, J = 1.4 Hz, |
| | | | amine | | 1H), 7.16 (t, J = 5.6 Hz, |
| | | | | | 1H), 6.95 (d, J = 2.6 Hz, 1H), |
| | | | | | 6.47 (d, J = 0.6 Hz, 1H), 4.64 (d, |
| | | | | | J = 5.6 Hz, 2H), 4.07 (s, 3H), |
| | | | | | 3.81 (s, 3H). |
| 244 | 228 | 458.53 | 5-({[8-(1-methyl-1H- | HPLC | 1H NMR (400 MHz, DMSO) δ |
| | | | indol-6-yl)quinoxalin- | 95.0%; | 8.88 (s, 1H), 8.69 (s, 1H), |
| | | | 6- | m/z = 459.2 | 8.64 (d, J = 1.9 Hz, 1H), 8.52 (d, J = 1.9 Hz, |
| | | | yl]amino}(pyridin-3- | [M + H] | 1H), 8.25 (d, J = 8.1 Hz, |
| | | | yl)methyl)-1,2- | | 1H), 7.76 (dd, J = 7.9, 5.2 Hz, |
| | | | dihydropyridin-2- | | 1H), 7.61 (d, J = 8.2 Hz, 2H), |
| | | | one | | 7.57 (d, J = 2.6 Hz, 1H), |
| | | | | | 7.53 (dd, J = 9.5, 2.7 Hz, 1H), 7.48 (t, |
| | | | | | J = 5.5 Hz, 2H), 7.40 (d, J = 3.1 Hz, |
| | | | | | 1H), 7.28 (dd, J = 8.1, 1.5 Hz, |
| | | | | | 1H), 6.84 (d, J = 2.5 Hz, 1H), |
| | | | | | 6.48 (dd, J = 3.1, 0.8 Hz, 1H), |
| | | | | | 6.40 (d, J = 9.5 Hz, 1H), 6.01 (d, |
| | | | | | J = 5.8 Hz, 1H), 3.82 (s, 3H). |
| 245 | 229 | 516.65 | N-[(1- | HPLC | 1H NMR (400 MHz, DMSO) δ |
| | | | cyclopropanecarbonylpiperidin- | 96.5%; | 8.72 (d, J = 1.8 Hz, 1H), 8.57 (d, |
| | | | 4- | m/z = 517.2 | J = 1.8 Hz, 1H), 8.45 (dd, J = 4.8, |
| | | | yl)(pyridin-3- | [M + H] | 1.5 Hz, 1H), 8.42 (d, J = 1.9 Hz, |
| | | | yl)methyl]-8-(1- | | 1H), 7.86 (d, J = 7.9 Hz, 1H), |
| | | | methyl-1H-indol-6- | | 7.60 (d, J = 7.7 Hz, 2H), 7.51 (s, |
| | | | yl)quinoxalin-6- | | 1H), 7.38 (dd, J = 7.1, 4.0 Hz, |
| | | | amine | | 2H), 7.26 (d, J = 8.2 Hz, 2H), |
| | | | | | 6.71 (s, 1H), 6.48 (d, J = 3.1 Hz, |
| | | | | | 1H), 4.56 (d, J = 7.0 Hz, 1H), |
| | | | | | 4.44 (dd, J = 36.1, 12.6 Hz, 2H), |
| | | | | | 3.98-3.85 (m, 2H), 3.82 (s, |
| | | | | | 3H), 3.05-2.88 (m, 2H), |
| | | | | | 2.26 (dd, J = 15.0, 7.5 Hz, 2H), |
| | | | | | 1.50 (dd, J = 14.7, 7.4 Hz, 2H), |
| | | | | | 0.91-0.85 (m, 4H). |
| 246 | 230 | 442.53 | 8-(1-methyl-1H- | HPLC | 1H NMR (400 MHz, DMSO) δ |
| | | | indol-6-yl)-N- | 97.8%; | 8.77 (d, J = 1.9 Hz, 1H), 8.62 (d, |
| | | | [pyridin-3-yl(pyridin- | m/z = 443.1 | J = 1.9 Hz, 1H), 8.59 (d, J = 5.9 Hz, |
| | | | 4- | [M + H] | 2H), 8.54-8.51 (m, 1H), |
| | | | yl)methyl]quinoxalin- | | 8.50 (d, J = 1.9 Hz, 1H), 7.88 (d, |
| | | | 6-amine | | J = 8.0 Hz, 1H), 7.61 (d, J = 9.2 Hz, |
| | | | | | 4H), 7.55 (d, J = 6.0 Hz, 2H), |
| | | | | | 7.44 (dd, J = 8.0, 4.8 Hz, 1H), |
| | | | | | 7.39 (d, J = 3.1 Hz, 1H), |
| | | | | | 7.28 (dd, J = 8.2, 1.4 Hz, 1H), |
| | | | | | 6.83 (d, J = 2.5 Hz, 1H), 6.48 (d, J = 2.9 Hz, |
| | | | | | 1H), 6.15 (d, J = 7.1 Hz, |
| | | | | | 1H), 3.82 (s, 3H). |
| 247 | 231 | 504.64 | 1-[4-({[8-(1-methyl- | HPLC | 1H NMR (400 MHz, DMSO) δ |
| | | | 1H-indol-6- | 95.0%; | 8.71 (d, J = 1.9 Hz, 1H), 8.57 (d, |
| | | | yl)quinoxalin-6- | m/z = 505.2 | J = 1.9 Hz, 1H), 8.44 (dd, J = 4.7, |
| | | | yl]amino}(pyridin-3- | [M + H] | 1.5 Hz, 1H), 8.42 (d, J = 1.9 Hz, |
| | | | yl)methyl)piperidin- | | 1H), 7.86 (d, J = 8.0 Hz, 1H), |
| | | | 1- | | 7.62-7.58 (m, 2H), 7.51 (d, J = 2.6 Hz, |
| | | | yl]propan-2-one | | 1H), 7.40-7.35 (m, |
| | | | | | 2H), 7.25 (dd, J = 8.3, 1.1 Hz, |
| | | | | | 2H), 6.70 (d, J = 2.4 Hz, 1H), |
| | | | | | 6.48 (d, J = 3.0 Hz, 1H), 4.52 (t, |
| | | | | | J = 7.9 Hz, 1H), 3.82 (s, 3H), |
| | | | | | 3.11 (s, 2H), 2.86 (d, J = 11.0 Hz, |
| | | | | | 1H), 2.76 (d, J = 11.2 Hz, |
| | | | | | 1H), 2.07 (s, 3H), 2.00 (t, J = 10.5 Hz, |
| | | | | | 2H), 1.91 (t, J = 10.6 Hz, |
| | | | | | 1H), 1.72 (s, 1H), |
| | | | | | 1.48-1.34 (m, 3H). |
| 248 | 232 | 518.67 | 1-[4-({[8-(1-methyl- | HPLC | 1H NMR (400 MHz, DMSO) δ |
| | | | 1H-indol-6- | 95.7%; | 8.72 (d, J = 1.8 Hz, 1H), 8.57 (d, |
| | | | yl)quinoxalin-6- | m/z = 519.3 | J = 1.8 Hz, 1H), 8.45 (dd, J = 4.8, |
| | | | yl]amino}(pyridin-3- | [M + H] | 1.5 Hz, 1H), 8.42 (d, J = 1.9 Hz, |
| | | | yl)methyl)piperidin- | | 1H), 7.86 (d, J = 7.9 Hz, 1H), |
| | | | 1-yl]butan-1- | | 7.60 (d, J = 7.7 Hz, 2H), 7.51 (s, |
| | | | one | | 1H), 7.38 (dd, J = 7.1, 4.0 Hz, |
| | | | | | 2H), 7.26 (d, J = 8.2 Hz, 2H), |
| | | | | | 6.71 (s, 1H), 6.48 (d, J = 3.1 Hz, |
| | | | | | 1H), 4.56 (d, J = 7.0 Hz, 1H), |
| | | | | | 4.49 (d, J = 12.8 Hz, 1H), |
| | | | | | 4.40 (d, J = 12.5 Hz, 1H), |
| | | | | | 4.01-3.84 (m, 1H), 3.82 (s, 3H), |
| | | | | | 3.03-2.85 (m, 2H), 2.26 (dd, J = 15.0, |
| | | | | | 7.5 Hz, 2H), 2.01 (s, 2H), |
| | | | | | 1.50 (dd, J = 14.7, 7.4 Hz, 2H), |
| | | | | | 1.32 (d, J = 13.7 Hz, 2H), 0.86 (dt, J = 11.3, |
| | | | | | 7.4 Hz, 3H). |
| 249 | 233 | 490.61 | 1-[3-({[8-(1-methyl- | HPLC | 1H NMR (400 MHz, DMSO) δ |
| | | | 1H-indol-6- | 88.2%; | 8.75 (d, J = 15.9 Hz, 1H), |
| | | | yl)quinoxalin-6- | m/z = 491.2 | 8.58 (s, 1H), 8.43 (s, 2H), 7.90 (dd, J = 13.4, |
| | | | yl]amino}(pyridin-3- | [M + H] | 7.9 Hz, 1H), 7.59 (d, J = 7.4 Hz, |
| | | | yl)methyl)piperidin- | | 2H), 7.50-7.45 (m, |
| | | | 1-yl]ethan-1- | | 1H), 7.36 (dt, J = 25.2, 7.4 Hz, |
| | | | one | | 3H), 7.25 (d, J = 8.2 Hz, 1H), |
| | | | | | 6.81 (d, J = 7.4 Hz, 1H), 6.47 (d, |
| | | | | | J = 3.0 Hz, 1H), 4.58 (q, J = 8.8 Hz, |
| | | | | | 1H), 3.97 (dd, J = 27.7, 12.9 Hz, |
| | | | | | 1H), 3.85-3.79 (m, 3H), |
| | | | | | 3.74 (d, J = 13.1 Hz, 1H), |
| | | | | | 3.29-3.14 (m, 1H), 2.94 (dd, J = 24.1, |
| | | | | | 11.3 Hz, 1H), 2.57 (d, J = 11.9 Hz, |
| | | | | | 1H), 2.06-1.99 (m, 3H), |
| | | | | | 1.95-1.59 (m, 2H), |
| | | | | | 1.43-1.29 (m, 2H). |
| 250 | 234 | 490.61 | 1-[3-({[8-(1-methyl- | HPLC | 1H NMR (400 MHz, DMSO) δ |
| | | | 1H-indol-6- | 90.1%; | 8.79 (d, J = 6.6 Hz, 1H), 8.58 (s, |
| | | | yl)quinoxalin-6- | m/z = 491.2 | 1H), 8.52 (d, J = 4.8 Hz, 1H), |
| | | | yl]amino}(pyridin-3- | [M + H] | 8.44 (dd, J = 4.0, 1.8 Hz, 1H), |
| | | | yl)methyl)piperidin- | | 8.01 (d, J = 7.7 Hz, 1H), 7.59 (d, |
| | | | 1-yl]ethan-1- | | J = 8.0 Hz, 2H), 7.55-7.44 (m, |
| | | | one | | 2H), 7.38 (d, J = 3.0 Hz, 1H), |
| | | | | | 7.31-7.18 (m, 2H), 6.77 (s, |
| | | | | | 1H), 6.47 (d, J = 3.0 Hz, 1H), |
| | | | | | 4.63 (t, J = 8.4 Hz, 1H), 4.30 (d, |
| | | | | | J = 12.5 Hz, 1H), 3.98 (d, J = 10.2 Hz, |
| | | | | | 1H), 3.81 (s, 3H), |
| | | | | | 3.74 (s, 1H), 3.49 (d, J = 12.8 Hz, |
| | | | | | 2H), 2.97 (dd, J = 26.5, 12.3 Hz, |
| | | | | | 1H), 2.23 (dd, J = 61.6, 13.3 Hz, |
| | | | | | 1H), 1.97 (s, 3H), |
| | | | | | 1.55-1.33 (m, 2H). |
| 251 | 235 | 390.45 | 3-({[8-(1-methyl-1H- | HPLC | 1H NMR (400 MHz, DMSO) δ |
| | | | indol-6-yl)quinoxalin- | 98.7%; | 8.94 (s, 1H), 8.76 (d, J = 5.0 Hz, |
| | | | 6- | m/z = 391.1 | 1H), 8.65 (d, J = 1.8 Hz, 1H), |
| | | | yl]amino}methyl)pyridine- | [M + H] | 8.51 (d, J = 1.9 Hz, 1H), 7.93 (d, |
| | | | 4-carbonitrile | | J = 4.9 Hz, 1H), 7.64 (s, 1H), |
| | | | | | 7.61 (d, J = 8.2 Hz, 1H), 7.47 (d, |
| | | | | | J = 2.6 Hz, 1H), 7.39 (d, J = 3.1 Hz, |
| | | | | | 1H), 7.36 (d, J = 5.7 Hz, 1H), |
| | | | | | 7.29 (dd, J = 8.2, 1.4 Hz, 1H), |
| | | | | | 6.87 (d, J = 2.6 Hz, 1H), |
| | | | | | 6.48 (dd, J = 2.9, 0.4 Hz, 1H), |
| | | | | | 4.73 (d, J = 5.6 Hz, 2H), 3.82 (s, 3H). |
| 252 | 236 | 506.61 | 2-[4-({[8-(1-methyl- | HPLC | 1H NMR (400 MHz, DMSO) δ |
| | | | 1H-indol-6- | 90.5%; | 8.73-8.69 (m, J = 1.6 Hz, 1H), |
| | | | yl)quinoxalin-6- | m/z = 507.2 | 8.56 (d, J = 1.9 Hz, 1H), |
| | | | yl]amino}(pyridin-3- | [M + H] | 8.43 (dd, J = 4.7, 1.5 Hz, 1H), |
| | | | yl)methyl)piperidin- | | 8.41 (d, J = 1.9 Hz, 1H), 7.85 (d, J = 7.9 Hz, |
| | | | 1- | | 1H), 7.58 (d, J = 8.1 Hz, |
| | | | yl]acetic acid | | 2H), 7.51 (d, J = 2.5 Hz, 1H), |
| | | | | | 7.37 (dd, J = 8.6, 3.9 Hz, 2H), |
| | | | | | 7.29 (d, J = 7.8 Hz, 1H), |
| | | | | | 7.27-7.22 (m, 1H), 6.69 (d, J = 2.2 Hz, |
| | | | | | 1H), 6.46 (d, J = 3.1 Hz, 1H), |
| | | | | | 4.50 (t, J = 7.9 Hz, 1H), 3.81 (s, |
| | | | | | 3H), 3.05 (dd, J = 34.8, 12.4 Hz, |
| | | | | | 2H), 2.93 (s, 2H), |
| | | | | | 2.30-2.12 (m, 3H), 1.99 (d, J = 11.7 Hz, |
| | | | | | 1H), 1.56-1.38 (m, J = 34.9, |
| | | | | | 8.9 Hz, 3H). |
| 253 | 237 | 505.63 | 2-[4-({[8-(1-methyl- | HPLC | not determined |
| | | | 1H-indol-6- | 89.2%; | |
| | | | yl)quinoxalin-6- | m/z = 506.3 | |
| | | | yl]amino}(pyridin-3- | [M + H] | |
| | | | yl)methyl)piperidin- | | |
| | | | 1- | | |
| | | | yl]acetamide | | |
| 254 | 238 | 520.64 | 1-{4-[(6- | HPLC | not determined |
| | | | methoxypyridin-3- | 93.5%; | |
| | | | yl)({[8-(1-methyl-1H- | m/z = 521.2 | |
| | | | indol-6-yl)quinoxalin- | [M + H] | |
| | | | 6- | | |
| | | | yl]amino})methyl]piperidin- | | |
| | | | 1- | | |
| | | | yl}ethan-1-one | | |
| 255 | 239 | 520.64 | 2-methoxy-1-[4-({[8- | HPLC | 1H NMR (400 MHz, DMSO) δ |
| | | | (1-methyl-1H-indol- | 95.7%; | 8.72 (s, 1H), 8.57 (d, J = 1.9 Hz, |
| | | | 6- | m/z = 521.2 | 1H), 8.45 (dd, J = 4.8, 1.4 Hz, |
| | | | yl)quinoxalin-6- | [M + H] | 1H), 8.42 (d, J = 1.9 Hz, 1H), |
| | | | yl]amino}(pyridin-3- | | 7.86 (d, J = 7.8 Hz, 1H), 7.60 (d, |
| | | | yl)methyl)piperidin- | | J = 7.7 Hz, 2H), 7.50 (s, 1H), |
| | | | 1-yl]ethan-1-one | | 7.38 (dt, J = 7.0, 3.6 Hz, 2H), |
| | | | | | 7.25 (dd, J = 8.3, 1.2 Hz, 2H), |
| | | | | | 6.71 (s, 1H), 6.48 (d, J = 3.0 Hz, |
| | | | | | 1H), 4.57 (d, J = 7.8 Hz, 1H), |
| | | | | | 4.38 (dd, J = 34.8, 11.7 Hz, 2H), |
| | | | | | 4.12-3.99 (m, 3H), |
| | | | | | 3.87-3.76 (m, 4H), 3.27 (s, J = 6.0 Hz, |
| | | | | | 3H), 3.05-2.85 (m, 1H), |
| | | | | | 2.03 (s, 2H), 1.38-1.29 (m, J = 10.9 Hz, |
| | | | | | 2H). |
| 257 | 241 | 443.51 | 8-(1-methyl-1H- | HPLC | 1H NMR (400 MHz, DMSO) δ |
| | | | indol-6-yl)-N- | 98.0%; | 8.90 (d, J = 1.8 Hz, 1H), 8.80 (d, |
| | | | [pyridin-3- | m/z = 444.2 | J = 1.8 Hz, 1H), 8.73 (s, 1H), |
| | | | yl(pyrimidin-5- | [M + H] | 8.63 (d, J = 4.0 Hz, 1H), 8.49 (s, |
| | | | yl)methyl]quinoxalin- | | 1H), 8.00 (d, J = 8.0 Hz, 1H), |
| | | | 6-amine | | 7.92 (d, J = 2.7 Hz, 1H), 7.77 (s, |
| | | | | | 1H), 7.71 (d, J = 2.7 Hz, 1H), |
| | | | | | 7.64 (d, J = 8.2 Hz, 1H), |
| | | | | | 7.58 (dd, J = 7.9, 4.9 Hz, 1H), |
| | | | | | 7.44-7.38 (m, 2H), 6.52-6.47 (m, |
| | | | | | 1H), 4.88 (s, 1H), 4.86 (d, J = 12.5 Hz, |
| | | | | | 1H), 4.45 (d, J = 12.4 Hz, |
| | | | | | 1H), 3.86 (s, 3H). |
| 258 | 242 | 489.60 | N-[(6- | HPLC | 1H NMR (400 MHz, DMSO) δ |
| | | | methoxypyridin-3- | 98.2%; | 8.92 (s, 1H), 8.71 (s, 1H), |
| | | | yl)(pyridin-3- | m/z = 490.1 | 8.66 (d, J = 1.9 Hz, 1H), 8.53 (d, J = 1.9 Hz, |
| | | | yl)methyl]-8- | [M + H] | 1H), 8.34 (d, J = 2.4 Hz, |
| | | | (3-methyl-1- | | 1H), 8.29 (d, J = 7.9 Hz, 1H), |
| | | | benzothiophen-5- | | 8.05 (d, J = 8.3 Hz, 1H), 7.92 (d, |
| | | | yl)quinoxalin-6- | | J = 1.4 Hz, 1H), 7.82 (dd, J = 8.6, |
| | | | amine | | 2.5 Hz, 1H), 7.78 (dd, J = 7.7, |
| | | | | | 5.3 Hz, 1H), 7.62 (dd, J = 7.3, |
| | | | | | 2.1 Hz, 3H), 7.47 (d, J = 1.1 Hz, |
| | | | | | 1H), 6.92-6.85 (m, 2H), |
| | | | | | 6.25 (d, J = 5.6 Hz, 1H), 3.85 (s, |
| | | | | | 3H), 2.43 (d, J = 1.0 Hz, 3H). |
| 259 | 243 | 355.40 | 8-(1-methyl-1H- | HPLC | 1H NMR (400 MHz, DMSO) δ |
| | | | indol-6-yl)-N-(1,3- | 92.4%; | 8.98 (s, 1H), 8.67 (s, 1H), |
| | | | oxazol-5- | m/z = 356.1 | 8.66 (d, J = 1.9 Hz, 1H), 8.49 (d, J = 1.9 Hz, |
| | | | ylmethyl)quinoxalin- | [M + H] | 1H), 7.61 (d, J = 8.3 Hz, |
| | | | 6-amine | | 2H), 7.41 (d, J = 2.6 Hz, 1H), |
| | | | | | 7.39 (d, J = 3.1 Hz, 1H), |
| | | | | | 7.27 (dd, J = 8.1, 1.5 Hz, 1H), |
| | | | | | 6.91 (d, J = 2.6 Hz, 1H), 6.48 (dd, J = 3.1, |
| | | | | | 0.7 Hz, 1H), 4.38 (s, 2H), |
| | | | | | 3.82 (s, 3H). |
| 260 | 244 | 371.46 | 8-(1-methyl-1H- | HPLC | 1H NMR (400 MHz, DMSO) δ |
| | | | indol-6-yl)-N-(1,2- | 99.2%; | 8.98 (s, 1H), 8.65 (s, 1H), |
| | | | thiazol-4- | m/z = 372.1 | 8.64 (d, J = 1.9 Hz, 1H), 8.48 (d, J = 1.9 Hz, |
| | | | ylmethyl)quinoxalin- | [M + H] | 1H), 7.61 (d, J = 8.3 Hz, |
| | | | 6-amine | | 2H), 7.43 (d, J = 2.6 Hz, 1H), |
| | | | | | 7.39 (d, J = 3.0 Hz, 1H), |
| | | | | | 7.27 (dd, J = 8.1, 1.4 Hz, 1H), |
| | | | | | 7.24-7.12 (m, 1H), 6.88 (d, J = 2.6 Hz, |
| | | | | | 1H), 6.48 (d, J = 3.0 Hz, 1H), |
| | | | | | 4.60 (s, 2H), 3.82 (s, 3H). |
| 261 | 245 | 355.40 | 8-(1-methyl-1H- | HPLC | 1H NMR (400 MHz, DMSO) δ |
| | | | indol-6-yl)-N-(1,2- | 94.2%; | 8.66 (d, J = 1.9 Hz, 1H), 8.49 (d, |
| | | | oxazol-4- | m/z = 356.1 | J = 1.9 Hz, 1H), 8.34 (s, 1H), |
| | | | ylmethyl)quinoxalin- | [M + H] | 7.62 (s, 1H), 7.61 (d, J = 8.3 Hz, |
| | | | 6-amine | | 1H), 7.41 (d, J = 2.6 Hz, 1H), |
| | | | | | 7.39 (d, J = 3.1 Hz, 1H), |
| | | | | | 7.26 (dd, J = 8.1, 1.4 Hz, 1H), |
| | | | | | 7.20 (s, 1H), 7.17 (s, 1H), 6.98 (d, J = 2.6 Hz, |
| | | | | | 1H), 6.51-6.45 (m, |
| | | | | | 1H), 4.59 (s, 2H), 3.82 (s, 3H). |
| 262 | 246 | 371.46 | 8-(1-methyl-1H- | HPLC | 1H NMR (400 MHz, DMSO) δ |
| | | | indol-6-yl)-N-(1,3- | 92.3%; | 9.00 (d, J = 0.7 Hz, 1H), 8.65 (d, |
| | | | thiazol-5- | m/z = 372.1 | J = 1.9 Hz, 1H), 8.49 (d, J = 1.9 Hz, |
| | | | ylmethyl)quinoxalin- | [M + H] | 1H), 7.98 (d, J = 0.6 Hz, 1H), |
| | | | 6-amine | | 7.61 (d, J = 8.3 Hz, 2H), 7.41 (d, |
| | | | | | J = 2.6 Hz, 1H), 7.39 (d, J = 3.1 Hz, |
| | | | | | 1H), 7.29 (d, J = 5.9 Hz, 1H), |
| | | | | | 7.26 (dd, J = 8.1, 1.5 Hz, 1H), |
| | | | | | 6.95 (d, J = 2.6 Hz, 1H), |
| | | | | | 6.48 (dd, J = 3.0, 0.6 Hz, 1H), |
| | | | | | 4.77 (d, J = 5.9 Hz, 2H), 3.82 (s, 3H). |
| 263 | 247 | 475.57 | 5-({[8-(3-methyl-1- | HPLC | 1H NMR (400 MHz, DMSO) δ |
| | | | benzothiophen-5- | 99%%; | 8.88 (d, J = 1.6 Hz, 1H), |
| | | | yl)quinoxalin-6- | m/z = 476.1 | 8.68 (dd, J = 11.6, 3.0 Hz, 2H), |
| | | | yl]amino}(pyridin-3- | [M + H] | 8.53 (d, J = 1.9 Hz, 1H), 8.25 (d, J = 8.0 Hz, |
| | | | yl)methyl)-1,2- | | 1H), 8.05 (d, J = 8.3 Hz, |
| | | | dihydropyridin-2- | | 1H), 7.92 (d, J = 1.2 Hz, 1H), |
| | | | one | | 7.75 (dd, J = 7.9, 5.2 Hz, 1H), |
| | | | | | 7.66-7.58 (m, 2H), 7.53 (dd, J = 9.5, |
| | | | | | 2.7 Hz, 2H), |
| | | | | | 7.49-7.44 (m, 2H), 6.88 (d, J = 2.5 Hz, |
| | | | | | 1H), 6.40 (d, J = 9.5 Hz, 1H), |
| | | | | | 6.02 (d, J = 6.1 Hz, 1H), 2.43 (d, |
| | | | | | J = 1.0 Hz, 3H). |
| 264 | 248 | 505.63 | 2-amino-1-[4-({[8-(1- | HPLC | not determined |
| | | | methyl-1H-indol-6- | 99.6%; | |
| | | | yl)quinoxalin-6- | m/z = 506.3 | |
| | | | yl]amino}(pyridin-3- | [M + H] | |
| | | | yl)methyl)piperidin- | | |
| | | | 1-yl]ethan-1-one | | |
| 265 | 249 | 445.53 | N-[(1-methyl-1H- | HPLC | 1H NMR (400 MHz, DMSO) δ |
| | | | imidazol-5- | 98.4%; | 8.81 (d, J = 2.0 Hz, 1H), 8.62 (d, |
| | | | yl)(pyridin-3- | m/z = 446.2 | J = 1.9 Hz, 1H), 8.54 (dd, J = 4.7, |
| | | | yl)methyl]-8-(1- | [M + H] | 1.6 Hz, 1H), 8.48 (d, J = 1.9 Hz, |
| | | | methyl-1H-indol-6- | | 1H), 7.96 (dt, J = 7.9, 1.9 Hz, |
| | | | yl)quinoxalin-6- | | 1H), 7.69 (s, 1H), |
| | | | amine | | 7.62-7.58 (m, 2H), 7.55 (d, J = 2.6 Hz, |
| | | | | | 1H), 7.52 (d, J = 7.6 Hz, 1H), |
| | | | | | 7.45 (dd, J = 7.8, 4.8 Hz, 1H), |
| | | | | | 7.39 (d, J = 3.1 Hz, 1H), |
| | | | | | 7.26 (dd, J = 8.3, 1.3 Hz, 1H), |
| | | | | | 6.91 (d, J = 2.5 Hz, 1H), 6.48 (dd, J = 3.0, |
| | | | | | 0.6 Hz, 1H), 6.33 (s, 1H), |
| | | | | | 6.18 (d, J = 7.5 Hz, 1H), 3.81 (s, |
| | | | | | 3H), 3.68 (s, 3H). |
| 266 | 250 | 494.60 | 1-{4-[(1-methyl-1H- | HPLC | 1H NMR (400 MHz, DMSO) δ |
| | | | 1,2,3-triazol-5- | 94.8%; | 8.62 (s, 1H), 8.47 (d, J = 1.9 Hz, |
| | | | yl)({[8-(1- | m/z = 495.2 | 1H), 7.73 (s, 1H), |
| | | | methyl-1H-indol-6- | [M + H] | 7.64-7.57 (m, 2H), 7.48 (s, 1H), 7.38 (d, J = 3.1 Hz, |
| | | | yl)quinoxalin-6- | | 1H), 7.26 (dd, J = 8.2, |
| | | | yl]amino})methyl]piperidin- | | 1.4 Hz, 1H), 7.05 (d, J = 4.9 Hz, |
| | | | 1-yl}ethan-1- | | 1H), 6.85 (d, J = 4.0 Hz, 1H), |
| | | | one | | 6.47 (d, J = 2.6 Hz, 1H), 4.94 (d, |
| | | | | | J = 3.4 Hz, 1H), 4.43 (dd, J = 24.3, |
| | | | | | 13.3 Hz, 1H), 4.12 (s, 3H), |
| | | | | | 3.94-3.85 (m, 1H), 3.81 (s, |
| | | | | | 3H), 3.03 (d, J = 12.3 Hz, 1H), |
| | | | | | 2.54 (s, 1H), 2.15 (s, 2H), |
| | | | | | 1.98 (s, 4H), 1.41 (d, J = 12.5 Hz, |
| | | | | | 2H). |
| 267 | 251 | 458.53 | 4-({[8-(1-methyl-1H- | HPLC | 1H NMR (400 MHz, DMSO) δ |
| | | | indol-6-yl)quinoxalin- | 97.2%; | 11.54 (s, 1H), 8.77 (d, J = 1.9 Hz, |
| | | | 6- | m/z = 459.2 | 1H), 8.64 (d, J = 1.9 Hz, 1H), |
| | | | yl]amino}(pyridin-3- | [M + H] | 8.53 (dt, J = 4.9, 2.5 Hz, 1H), |
| | | | yl)methyl)-1,2- | | 8.50 (d, J = 1.9 Hz, 1H), |
| | | | dihydropyridin-2- | | 7.92-7.87 (m, 1H), 7.63 (s, 1H), |
| | | | one | | 7.60 (dd, J = 5.4, 2.7 Hz, 2H), |
| | | | | | 7.49-7.42 (m, 2H), 7.37 (dd, J = 7.8, |
| | | | | | 4.9 Hz, 2H), 7.28 (dd, J = 8.2, |
| | | | | | 1.5 Hz, 1H), 6.87 (d, J = 2.5 Hz, |
| | | | | | 1H), 6.52 (d, J = 0.8 Hz, 1H), |
| | | | | | 6.47 (dd, J = 3.1, 0.8 Hz, 1H), |
| | | | | | 6.28 (dd, J = 6.8, 1.7 Hz, 1H), |
| | | | | | 5.89 (d, J = 7.3 Hz, 1H), 3.81 (s, |
| | | | | | 3H). |
| 268 | 252 | 465.62 | 8-(3-methyl-1- | HPLC | not determined |
| | | | benzothiophen-5-yl)- | 99.0%; | |
| | | | N-[piperidin-4- | m/z = 466.2 | |
| | | | yl(pyridin-3- | [M + H] | |
| | | | yl)methyl]quinoxalin- | | |
| | | | 6-amine | | |
| 269 | 253 | 504.64 | N-[4-({[8-(1-methyl- | HPLC | not determined |
| | | | 1H-indol-6- | 93.1%; | |
| | | | yl)quinoxalin-6- | m/z = 505.2 | |
| | | | yl]amino}(pyridin-3- | [M + H] | |
| | | | yl)methyl)cyclohexyl]acetamide | | |
| 270 | 254 | 507.66 | 1-[4-({[8-(3-methyl- | HPLC | 1H NMR (400 MHz, CDCl3) δ |
| | | | 1-benzothiophen-5- | 89.2%; | 8.69 (s, 1H), 8.59 (s, 1H), |
| | | | yl)quinoxalin-6- | m/z = 508.2 | 8.55 (d, J = 3.7 Hz, 1H), 8.52 (d, J = 1.8 Hz, |
| | | | yl]amino}(pyridin-3- | [M + H] | 1H), 7.93 (d, J = 8.2 Hz, |
| | | | yl)methyl)piperidin- | | 1H), 7.88 (s, 1H), 7.71 (d, J = 7.8 Hz, |
| | | | 1-yl]ethan-1-one | | 1H), 7.57 (d, J = 8.3 Hz, |
| | | | | | 1H), 7.29 (ddd, J = 6.3, 5.8, 3.3 Hz, |
| | | | | | 2H), 7.12 (s, 1H), 6.82 (t, J = 2.3 Hz, |
| | | | | | 1H), 4.74 (dd, J = 34.1, |
| | | | | | 16.5 Hz, 2H), 4.48 (s, 1H), |
| | | | | | 3.88 (m, 1H), 3.03 (dd, J = 23.8, 11.3 Hz, |
| | | | | | 1H), 2.49 (m, 5H), 2.08 (s, |
| | | | | | 4H), 1.98 (dd, J = 17.9, 9.9 Hz, |
| | | | | | 3H). |
| 271 | 255 | 472.55 | N-[(S)-(6- | HPLC | 1H NMR (400 MHz, DMSO) δ |
| | | | methoxypyridin-3- | 96.6%; | 8.75 (d, J = 2.0 Hz, 1H), 8.62 (d, |
| | | | yl)(pyridin-3- | 473.2 | J = 1.9 Hz, 1H), 8.52-8.48 (m, |
| | | | yl)methyl]-8-(1- | m/z = [M + H] | 2H), 8.33 (d, J = 2.3 Hz, 1H), |
| | | | methyl-1H-indol-6- | | 7.88 (d, J = 8.0 Hz, 1H), |
| | | | yl)quinoxalin-6- | | 7.79 (dd, J = 8.6, 2.5 Hz, 1H), |
| | | | amine | | 7.63-7.59 (m, 3H), 7.51 (d, J = 7.0 Hz, |
| | | | | | 1H), 7.42 (dd, J = 7.9, 4.8 Hz, |
| | | | | | 1H), 7.39 (d, J = 3.0 Hz, 1H), |
| | | | | | 7.28 (dd, J = 8.2, 1.3 Hz, 1H), |
| | | | | | 6.86 (d, J = 8.6 Hz, 1H), 6.83 (d, |
| | | | | | J = 2.5 Hz, 1H), 6.48 (d, J = 3.0 Hz, |
| | | | | | 1H), 6.10 (d, J = 6.9 Hz, 1H), |
| | | | | | 3.84 (s, 3H), 3.82 (s, 3H). |
| 272 | 256 | 472.55 | N-[(R)-(6- | HPLC | 1H NMR (400 MHz, DMSO) δ |
| | | | methoxypyridin-3- | 98.2%; | 8.75 (d, J = 1.9 Hz, 1H), 8.62 (d, |
| | | | yl)(pyridin-3- | m/z = 473.2 | J = 1.8 Hz, 1H), 8.52-8.48 (m, |
| | | | yl)methyl]-8-(1- | [M + H] | 2H), 8.33 (d, J = 2.3 Hz, 1H), |
| | | | methyl-1H-indol-6- | | 7.88 (d, J = 7.9 Hz, 1H), |
| | | | yl)quinoxalin-6- | | 7.79 (dd, J = 8.6, 2.5 Hz, 1H), |
| | | | amine | | 7.61 (d, J = 8.3 Hz, 3H), 7.51 (d, J = 7.0 Hz, |
| | | | | | 1H), 7.42 (dd, J = 7.8, |
| | | | | | 4.9 Hz, 1H), 7.39 (d, J = 3.0 Hz, |
| | | | | | 1H), 7.28 (dd, J = 8.2, 1.4 Hz, |
| | | | | | 1H), 6.86 (d, J = 8.6 Hz, 1H), |
| | | | | | 6.83 (d, J = 2.4 Hz, 1H), 6.48 (d, |
| | | | | | J = 3.0 Hz, 1H), 6.10 (d, J = 6.9 Hz, |
| | | | | | 1H), 3.84 (s, 3H), 3.82 (s, |
| | | | | | 3H). |
| 273 | 257 | 450.55 | N,N-dimethyl-3-{[8- | HPLC | 1H NMR (400 MHz, DMSO) δ |
| | | | (1-methyl-1H-indol- | 95.9%; | 8.75 (s, 1H), 8.58 (d, J = 1.8 Hz, |
| | | | 6- | m/z = 451.2 | 1H), 8.44 (d, J = 1.8 Hz, 2H), |
| | | | yl)quinoxalin-6- | [M + H] | 7.91 (d, J = 7.8 Hz, 1H), 7.59 (d, |
| | | | yl]amino}-3-(pyridin- | | J = 8.5 Hz, 2H), 7.38 (m, 3H), |
| | | | 3- | | 7.24 (dd, J = 12.7, 8.4 Hz, 2H), |
| | | | yl)propanamide | | 6.67 (d, J = 2.2 Hz, 1H), 6.47 (d, |
| | | | | | J = 2.8 Hz, 1H), 5.12 (m, 1H), |
| | | | | | 3.81 (s, 3H), 3.06 (dd, J = 15.9, |
| | | | | | 7.7 Hz, 1H), 2.96 (s, 3H), |
| | | | | | 2.84 (m, 4H). |
| 274 | 258 | 519.65 | 2-amino-1-[4-({[8-(1- | HPLC | not determined |
| | | | methyl-1H-indol-6- | 91.7%; | |
| | | | yl)quinoxalin-6- | m/z = 520.3 | |
| | | | yl]amino}(pyridin-3- | [M + H] | |
| | | | yl)methyl)piperidin- | | |
| | | | 1-yl]propan-1-one | | |
| 275 | 259 | 519.65 | N-methyl-2-[4-({[8- | HPLC | 1H NMR (400 MHz, DMSO) δ |
| | | | (1-methyl-1H-indol- | 91.3%; | 8.71 (s, 1H), 8.57 (d, J = 1.9 Hz, |
| | | | 6- | m/z = 520.3 | 1H), 8.43 (m, 2H), 7.86 (d, J = 7.9 Hz, |
| | | | yl)quinoxalin-6- | [M + H] | 1H), 7.59 (dd, J = 8.9, |
| | | | yl]amino}(pyridin-3- | | 5.5 Hz, 3H), 7.50 (d, J = 2.6 Hz, |
| | | | yl)methyl)piperidin- | | 1H), 7.38 (dd, J = 7.7, 3.9 Hz, |
| | | | 1-yl]acetamide | | 2H), 7.25 (dd, J = 8.2, 1.2 Hz, |
| | | | | | 2H), 6.70 (d, J = 2.4 Hz, 1H), |
| | | | | | 6.48 (dd, J = 3.0, 0.7 Hz, 1H), |
| | | | | | 4.47 (t, J = 7.8 Hz, 1H), 3.82 (s, |
| | | | | | 3H), 2.86 (m, 3H), 2.78 (d, J = 11.1 Hz, |
| | | | | | 1H), 2.60 (d, J = 4.7 Hz, |
| | | | | | 3H), 2.00 (m, 3H), 1.74 (s, |
| | | | | | 1H), 1.39 (m, 3H). |
| 276 | 260 | 533.68 | N,N-dimethyl-2-[4- | HPLC | 1H NMR (400 MHz, DMSO) δ |
| | | | ({[8-(1-methyl-1H- | 95.9%; | 8.71 (d, J = 1.6 Hz, 1H), 8.56 (d, |
| | | | indol-6- | m/z = 534.3 | J = 1.9 Hz, 1H), 8.43 (m, 2H), |
| | | | yl)quinoxalin-6- | [M + H] | 7.85 (d, J = 8.0 Hz, 1H), |
| | | | yl]amino}(pyridin-3- | | 7.60 (m, 2H), 7.51 (d, J = 2.6 Hz, |
| | | | yl)methyl)piperidin- | | 1H), 7.37 (m, 2H), 7.25 (m, 2H), |
| | | | 1-yl]acetamide | | 6.70 (d, J = 2.3 Hz, 1H), 6.48 (d, |
| | | | | | J = 3.1 Hz, 1H), 4.51 (t, J = 7.9 Hz, |
| | | | | | 1H), 3.82 (s, 3H), 3.08 (s, |
| | | | | | 2H), 3.01 (s, 3H), 2.86 (m, 5H), |
| | | | | | 1.95 (dd, J = 28.5, 17.3 Hz, 3H), |
| | | | | | 1.72 (s, 1H), 1.35 (m, 3H). |
| 277 | 261 | 561.73 | N,N-diethyl-2-[4-({[8- | HPLC | 1H NMR (400 MHz, DMSO) δ |
| | | | (1-methyl-1H-indol- | 99.2%; | 8.72 (d, J = 1.7 Hz, 1H), 8.57 (d, |
| | | | 6- | m/z = 562.4 | J = 1.9 Hz, 1H), 8.43 (m, 2H), |
| | | | yl)quinoxalin-6- | [M + H] | 7.86 (d, J = 7.9 Hz, 1H), |
| | | | yl]amino}(pyridin-3- | | 7.59 (m, 2H), 7.50 (d, J = 2.6 Hz, |
| | | | yl)methyl)piperidin- | | 1H), 7.37 (m, 2H), 7.25 (m, 2H), |
| | | | 1-yl]acetamide | | 6.71 (d, J = 2.4 Hz, 1H), 6.47 (d, |
| | | | | | J = 3.1 Hz, 1H), 4.50 (t, J = 8.2 Hz, |
| | | | | | 1H), 4.05 (dt, J = 11.9, 10.4 Hz, |
| | | | | | 1H), 3.82 (s, 3H), 3.07 (s, |
| | | | | | 2H), 2.86 (dd, J = 40.0, 11.2 Hz, |
| | | | | | 3H), 1.97 (m, 5H), 1.72 (s, 1H), |
| | | | | | 1.36 (m, 3H), 1.11 (t, J = 7.0 Hz, |
| | | | | | 3H), 0.99 (t, J = 7.0 Hz, 3H). |
| 278 | 262 | 519.65 | 3-amino-1-[4-({[8-(1- | HPLC | not determined |
| | | | methyl-1H-indol-6- | 84.2%; | |
| | | | yl)quinoxalin-6- | m/z = 520.3 | |
| | | | yl]amino}(pyridin-3- | [M + H] | |
| | | | yl)methyl)piperidin- | | |
| | | | 1-yl]propan-1-one | | |
| 279 | 263 | 369.43 | 8-(1-methyl-1H- | HPLC | 1H NMR (400 MHz, DMSO) δ |
| | | | indol-6-yl)-N-[(4- | 94.1%; | 8.66 (d, J = 1.9 Hz, 1H), 8.49 (d, |
| | | | methyl-4H-1,2,4- | m/z = 370.2 | J = 1.9 Hz, 1H), 8.44 (s, 1H), |
| | | | triazol-3- | [M + H] | 7.60 (m, 2H), 7.44 (d, J = 2.6 Hz, |
| | | | yl)methyl]quinoxalin- | | 1H), 7.38 (d, J = 3.1 Hz, 1H), |
| | | | 6-amine | | 7.26 (dd, J = 8.1, 1.4 Hz, 1H), |
| | | | | | 7.20 (t, J = 5.6 Hz, 1H), 7.08 (d, |
| | | | | | J = 2.6 Hz, 1H), 6.47 (d, J = 3.1 Hz, |
| | | | | | 1H), 4.65 (d, J = 5.5 Hz, 2H), |
| | | | | | 3.81 (s, 3H), 3.71 (s, 3H). |
| 280 | 264 | 385.49 | N-[(3-methyl-1,2-thiazol- | HPLC | 1H NMR (400 MHz, DMSO) δ |
| | | | 5-yl)methyl]-8-(1- | 99.0%; | 8.64 (d, J = 1.9 Hz, 1H), 8.49 (d, |
| | | | methyl- | m/z = 386.2 | J = 1.9 Hz, 1H), 7.61 (m, 2H), |
| | | | 1H-indol-6-yl)quinoxalin- | [M + H] | 7.41 (m, 3H), 7.26 (dd, J = 8.1, |
| | | | 6-amine | | 1.4 Hz, 1H), 7.20 (s, 1H), |
| | | | | | 6.86 (d, J = 2.6 Hz, 1H), 6.48 (m, |
| | | | | | 1H), 4.81 (d, J = 5.9 Hz, 2H), |
| | | | | | 3.82 (s, 3H), 2.39 (s, 3H). |
| 281 | 265 | 371.46 | 8-(1-methyl-1H-indol- | HPLC | 1H NMR (400 MHz, DMSO) δ |
| | | | 6-yl)-N-[(1,2-thiazol- | 87.7%; | 8.65 (d, J = 1.9 Hz, 1H), 8.51 (t, |
| | | | 5- | m/z = 372.1 | J = 1.6 Hz, 2H), 7.63 (s, 1H), |
| | | | yl)methyl]quinoxalin- | [M + H] | 7.61 (d, J = 8.2 Hz, 1H), 7.45 (d, |
| | | | 6-amine | | J = 1.6 Hz, 2H), 7.43 (d, J = 2.6 Hz, |
| | | | | | 1H), 7.39 (d, J = 3.1 Hz, 1H), |
| | | | | | 7.27 (dd, J = 8.1, 1.4 Hz, 1H), |
| | | | | | 6.89 (d, J = 2.6 Hz, 1H), |
| | | | | | 6.49-6.47 (m, 1H), 4.89 (d, J = 6.0 Hz, |
| | | | | | 2H), 3.82 (s, 3H). |
| 282 | 266 | 370.42 | N-[(5-methyl-1,3,4- | HPLC | 1H NMR (400 MHz, DMSO) δ |
| | | | oxadiazol-2- | 97.5%; | 8.68 (d, J = 1.8 Hz, 1H), 8.52 (d, |
| | | | yl)methyl]-8-(1- | m/z = 371.2 | J = 1.9 Hz, 1H), 7.63 (s, 1H), |
| | | | methyl-1H-indol-6- | [M + H] | 7.61 (d, J = 8.2 Hz, 1H), 7.43 (d, |
| | | | yl)quinoxalin-6- | | J = 2.6 Hz, 1H), 7.39 (d, J = 3.0 Hz, |
| | | | amine | | 1H), 7.35 (t, J = 6.4 Hz, 1H), |
| | | | | | 7.27 (dd, J = 8.2, 1.4 Hz, 1H), |
| | | | | | 6.99 (d, J = 2.6 Hz, 1H), 6.48 (d, |
| | | | | | J = 3.1 Hz, 1H), 4.75 (d, J = 6.2 Hz, |
| | | | | | 2H), 3.82 (s, 3H), 2.49 (s, |
| | | | | | 3H). |
| 283 | 267 | 369.43 | N-[(5-methyl-1H- | HPLC | 1H NMR (400 MHz, DMSO) δ |
| | | | 1,2,4-triazol-3- | 97.7%; | 13.59 (s, 1H), 13.43 (s, 1H), |
| | | | yl)methyl]-8-(1- | m/z = 370.3 | 8.65 (d, J = 1.9 Hz, 1H), 8.63 (d, |
| | | | methyl-1H-indol-6- | [M + H] | J = 1.9 Hz, 1H), 8.49 (d, J = 1.9 Hz, |
| | | | yl)quinoxalin-6- | | 1H), 8.45 (d, J = 1.9 Hz, 1H), |
| | | | amine | | 7.45 (d, J = 2.6 Hz, 1H), 7.44 (d, |
| | | | | | J = 2.7 Hz, 1H), 7.40 (s, 1H), |
| | | | | | 7.38 (d, J = 3.1 Hz, 1H), |
| | | | | | 7.28-7.20 (m, 2H), 7.12 (t, J = 5.8 Hz, |
| | | | | | 1H), 6.94 (d, J = 2.6 Hz, 1H), |
| | | | | | 6.84 (d, J = 2.5 Hz, 1H), 6.48 (d, |
| | | | | | J = 2.5 Hz, 1H), 4.53 (d, J = 5.5 Hz, |
| | | | | | 1H), 4.39 (d, J = 5.8 Hz, 2H), |
| | | | | | 3.82 (s, 3H), 2.34 (s, 3H), |
| | | | | | 2.24 (s, 1H). |
| 284 | 268 | 354.42 | N-(1H-imidazol-4- | HPLC | not determined |
| | | | ylmethyl)-8-(1- | 86.4%; | |
| | | | methyl-1H-indol-6- | m/z = 355.3 | |
| | | | yl)quinoxalin-6- | [M + H] | |
| | | | amine | | |
| 285 | 269 | 382.47 | N-[(1,2-dimethyl-1H- | HPLC | 1H NMR (400 MHz, DMSO) δ |
| | | | imidazol-5-yl)methyl]- | 99.7%; | 8.65 (d, J = 1.9 Hz, 1H), 8.48 (d, |
| | | | 8-(1- | m/z = 383.3 | J = 1.9 Hz, 1H), 8.15 (s, 1H), |
| | | | methyl-1H-indol-6-yl)quinoxalin- | [M + H] | 7.60 (d, J = 8.3 Hz, 2H), 7.42 (d, |
| | | | 6-amine | | J = 2.6 Hz, 1H), 7.39 (d, J = 3.1 Hz, |
| | | | | | 1H), 7.26 (dd, J = 8.1, 1.5 Hz, |
| | | | | | 1H), 6.98 (d, J = 2.6 Hz, 1H), |
| | | | | | 6.89 (s, 1H), 6.48 (dd, J = 3.0, |
| | | | | | 0.6 Hz, 1H), 4.41 (d, J = 5.1 Hz, |
| | | | | | 2H), 3.82 (s, 3H), 3.56 (s, 3H). |
| 286 | 270 | 355.41 | 8-(1-methyl-1H- | HPLC | 1H NMR (400 MHz, DMSO) δ |
| | | | indol-6-yl)-N-(4H- | 98.0%; | 13.93 (s, 1H), 8.64 (d, J = 1.8 Hz, |
| | | | 1,2,4-triazol-3- | m/z = 356.3 | 1H), 8.47 (d, J = 1.8 Hz, 1H), |
| | | | ylmethyl)quinoxalin- | [M + H] | 7.61 (d, J = 2.8 Hz, 1H), 7.60 (s, |
| | | | 6-amine | | 1H), 7.45 (d, J = 2.6 Hz, 1H), |
| | | | | | 7.39 (d, J = 3.0 Hz, 1H), |
| | | | | | 7.26 (dd, J = 8.3, 1.3 Hz, 1H), |
| | | | | | 7.22 (s, 1H), 6.91 (s, 1H), |
| | | | | | 6.51-6.46 (m, 1H), 4.53 (s, 2H), 3.82 (s, |
| | | | | | 3H). |
| 287 | 271 | 504.64 | 1-[4-({[8-(1-methyl- | HPLC | 1H NMR (400 MHz, DMSO) δ |
| | | | 1H-indol-6- | 95.9%; | 8.57 (d, J = 3.0 Hz, 2H), 8.42 (d, |
| | | | yl)quinoxalin-6- | m/z = 505.2 | J = 1.8 Hz, 1H), 8.28 (d, J = 4.9 Hz, |
| | | | yl]amino}(4- | [M + H] | 1H), 7.60 (d, J = 8.3 Hz, 2H), |
| | | | methylpyridin-3- | | 7.51 (s, 1H), 7.39 (d, J = 3.1 Hz, |
| | | | yl)methyl)piperidin- | | 1H), 7.27 (d, J = 1.4 Hz, 1H), |
| | | | 1- | | 7.24 (d, J = 7.2 Hz, 1H), 7.21 (d, |
| | | | yl]ethan-1-one | | J = 5.0 Hz, 1H), 6.55 (s, 1H), |
| | | | | | 6.48 (d, J = 3.0 Hz, 1H), 4.67 (d, |
| | | | | | J = 5.4 Hz, 1H), 4.51 (d, J = 12.5 Hz, |
| | | | | | 1H), 4.41 (d, J = 13.1 Hz, |
| | | | | | 1H), 3.94 (s, 1H), 3.82 (s, 3H), |
| | | | | | 3.51 (s, 1H), 2.98 (s, 1H), |
| | | | | | 2.55 (d, J = 3.8 Hz, 3H), 1.99 (d, J = 6.5 Hz, |
| | | | | | 3H), 1.40 (dd, J = 38.7, |
| | | | | | 18.9 Hz, 4H) |
| 288 | 272 | 457.54 | N-[(2-aminopyridin- | HPLC | 1H NMR (400 MHz, DMSO) δ |
| | | | 4-yl)(pyridin-3- | 85.8%; | 8.74 (d, J = 1.9 Hz, 1H), 8.62 (d, |
| | | | yl)methyl]-8-(1- | m/z = 458.3 | J = 1.9 Hz, 1H), 8.52 (dd, J = 4.8, |
| | | | methyl-1H-indol-6- | [M + H] | 1.6 Hz, 1H), 8.49 (d, J = 1.9 Hz, |
| | | | yl)quinoxalin-6- | | 1H), 7.89 (d, J = 5.3 Hz, 1H), |
| | | | amine | | 7.85 (d, J = 8.0 Hz, 1H), 7.61 (t, |
| | | | | | J = 3.8 Hz, 3H), 7.48 (d, J = 6.8 Hz, |
| | | | | | 1H), 7.43 (dd, J = 7.9, 4.8 Hz, |
| | | | | | 1H), 7.39 (d, J = 3.1 Hz, 1H), |
| | | | | | 7.28 (dd, J = 8.3, 1.3 Hz, 1H), |
| | | | | | 6.81 (d, J = 2.5 Hz, 1H), 6.60 (d, |
| | | | | | J = 5.3 Hz, 1H), 6.54 (s, 1H), |
| | | | | | 6.48 (d, J = 3.1 Hz, 1H), 5.97 (s, |
| | | | | | 2H), 5.86 (d, J = 6.7 Hz, 1H), |
| | | | | | 3.82 (s, 3H). |
| 289 | 273 | 462.56 | 1-[3-({[8-(1-methyl- | HPLC | 1H NMR (400 MHz, DMSO) δ |
| | | | 1H-indol-6- | 99.6%; | 8.83 (dd, J = 6.4, 1.9 Hz, 2H), |
| | | | yl)quinoxalin-6- | m/z = 463.1 | 8.60 (d, J = 1.9 Hz, 2H), |
| | | | yl]amino}(pyridin-3- | [M + H] | 8.50-8.43 (m, 4H), 7.96 (dt, J = 7.9, |
| | | | yl)methyl)azetidin-1- | | 1.8 Hz, 2H), 7.60 (d, J = 8.2 Hz, |
| | | | yl]ethan-1- | | 4H), 7.45 (d, J = 2.5 Hz, 2H), |
| | | | one | | 7.42-7.37 (m, 4H), 7.34 (dd, J = 8.2, |
| | | | | | 1.2 Hz, 2H), 7.26 (d, J = 8.3 Hz, |
| | | | | | 2H), 6.81 (t, J = 2.1 Hz, |
| | | | | | 2H), 6.48 (d, J = 3.0 Hz, 2H), |
| | | | | | 4.99 (dd, J = 17.9, 8.8 Hz, 2H), |
| | | | | | 4.29 (t, J = 8.3 Hz, 1H), |
| | | | | | 4.12-3.88 (m, 4H), 3.82 (s, 6H), |
| | | | | | 3.78 (dd, J = 9.7, 5.4 Hz, 1H), |
| | | | | | 3.74-3.62 (m, 2H), 3.08-2.98 (m, |
| | | | | | 2H), 1.76 (d, J = 7.9 Hz, 6H). |
| 290 | 274 | 445.53 | N-[(1-methyl-1H- | HPLC | not determined |
| | | | imidazol-4- | 87.7%; | |
| | | | yl)(pyridin-3- | m/z = 446.3 | |
| | | | yl)methyl]-8-(1- | [M + H] | |
| | | | methyl-1H-indol-6- | | |
| | | | yl)quinoxalin-6- | | |
| | | | amine | | |
| 291 | 275 | 539.66 | 1-[4-({[8-(2-amino- | HPLC | 1H NMR (400 MHz, DMSO) δ |
| | | | 1,3-benzothiazol-5- | 87.6%; | 8.58 (s, 1H), 8.42 (d, J = 1.9 Hz, |
| | | | yl)quinoxalin-6- | m/z = 540.1 | 1H), 8.27 (d, J = 2.2 Hz, 1H), |
| | | | yl]amino}(6- | [M + H] | 7.77-7.74 (m, 1H), 7.72 (d, J = 8.1 Hz, |
| | | | methoxypyridin-3- | | 1H), 7.52 (s, 2H), |
| | | | yl)methyl)piperidin- | | 7.47 (s, 1H), 7.33 (s, 1H), 7.21 (dd, J = 8.1, |
| | | | 1-yl]ethan-1-one | | 1.7 Hz, 1H), 7.17 (d, J = 8.3 Hz, |
| | | | | | 1H), 6.80 (d, J = 8.5 Hz, |
| | | | | | 1H), 6.71 (s, 1H), 4.46 (s, 1H), |
| | | | | | 4.39-4.34 (m, 1H),, 3.81 (s, |
| | | | | | 3H), 2.68 (d, J = 1.8 Hz, 1H), |
| | | | | | 2.33 (s, 1H), 1.98 (d, J = 5.8 Hz, |
| | | | | | 3H), 1.91 (s, 1H), 1.30 (s, 1H), |
| | | | | | 1.25-1.15 (m, 4H). |
| 292 | 276 | 516.44 | 1-[4-({[8-(4-bromophenyl)quinoxalin- | HPLC | not determined |
| | | | 6- | 93.9%; | |
| | | | yl]amino}(pyridin-3-yl)methyl)piperidin- | m/z = 516.3 | |
| | | | 1- | [M + H] | |
| | | | yl]ethan-1- | | |
| | | | one | | |
| 293 | 277 | 509.63 | 1-[4-({[8-(2-amino- | HPLC | 1H NMR (400 MHz, DMSO) δ |
| | | | 1,3-benzothiazol-5- | 91.3%; | 8.71 (d, J = 1.7 Hz, 1H), 8.58 (d, |
| | | | yl)quinoxalin-6- | m/z = 510.1 | J = 1.6 Hz, 1H), 8.44 (dd, J = 4.7, |
| | | | yl]amino}(pyridin-3- | [M + H] | 1.5 Hz, 1H), 8.42 (d, J = 1.9 Hz, |
| | | | yl)methyl)piperidin- | | 1H), 7.85 (d, J = 8.1 Hz, 1H), |
| | | | 1-yl]ethan-1-one | | 7.72 (d, J = 8.1 Hz, 1H), 7.52 (s, |
| | | | | | 2H), 7.50 (s, 1H), 7.50 (s, 1H), |
| | | | | | 7.37 (dd, J = 7.7, 4.9 Hz, 1H), |
| | | | | | 7.24 (d, J = 7.9 Hz, 1H), |
| | | | | | 7.22-7.18 (m, 1H), 6.71 (s, 1H), |
| | | | | | 4.56 (d, J = 7.5 Hz, 1H), 4.40 (d, J = 34.4 Hz, |
| | | | | | 2H), 3.94-3.79 (m, |
| | | | | | 2H), 3.51 (s, 1H), 3.00 (s, 2H), |
| | | | | | 1.98 (d, J = 6.0 Hz, 3H), 1.83 (s, |
| | | | | | 1H), 1.33 (s, 1H). |
| 294 | 278 | 520.64 | 5-[(1-acetylpiperidin- | HPLC | 1H NMR (400 MHz, DMSO) δ |
| | | | 4-yl)({[8-(1-methyl- | 99.1%; | 8.60 (d, J = 1.7 Hz, 1H), 8.44 (d, |
| | | | 1H-indol-6- | m/z = 521.4 | J = 1.8 Hz, 1H), 7.82 (s, 1H), |
| | | | yl)quinoxalin-6- | [M + H] | 7.62 (s, 1H), 7.60 (d, J = 8.3 Hz, |
| | | | yl]amino})methyl]-1- | | 1H), 7.51 (dd, J = 9.4, 2.3 Hz, |
| | | | methyl-1,2- | | 1H), 7.47 (s, 1H), 7.39 (d, J = 3.0 Hz, |
| | | | dihydropyridin-2-one | | 1H), 7.26 (dd, J = 8.1, |
| | | | | | 1.2 Hz, 1H), 7.05 (d, J = 7.8 Hz, |
| | | | | | 1H), 6.78 (s, 1H), 6.48 (d, J = 2.8 Hz, |
| | | | | | 1H), 6.41 (d, J = 9.3 Hz, |
| | | | | | 1H), 4.48-4.37 (m, 1H), |
| | | | | | 4.21 (d, J = 4.6 Hz, 1H), 3.88 (d, J = 14.8 Hz, |
| | | | | | 1H), 3.82 (s, 3H), |
| | | | | | 3.42 (s, 3H), 2.99 (dd, J = 23.6, 11.9 Hz, |
| | | | | | 1H), 2.05 (s, 1H), 1.99 (d, J = 3.0 Hz, |
| | | | | | 3H), 1.91 (s, 2H), |
| | | | | | 1.46 (d, J = 11.9 Hz, 1H), 1.14 (dd, J = 23.3, |
| | | | | | 11.6 Hz, 2H). |
| 295 | 279 | 491.57 | 8-(2-amino-1,3- | HPLC | 1H NMR (400 MHz, DMSO) δ |
| | | | benzothiazol-5-yl)-N- | 81.9%; | 8.75 (s, 1H), 8.63 (d, J = 1.9 Hz, |
| | | | [(6- | m/z = 492.3 | 1H), 8.49 (d, J = 1.9 Hz, 2H), |
| | | | methoxypyridin-3- | [M + H] | 8.32 (d, J = 2.4 Hz, 1H), 7.87 (s, |
| | | | yl)(pyridin-3- | | 1H), 7.78 (dd, J = 8.7, 2.5 Hz, |
| | | | yl)methyl]quinoxalin- | | 1H), 7.73 (d, J = 8.1 Hz, 1H), |
| | | | 6-amine | | 7.57 (d, J = 2.7 Hz, 1H), 7.53 (s, |
| | | | | | 3H), 7.43 (s, 1H), 7.23 (dd, J = 8.0, |
| | | | | | 1.7 Hz, 1H), 6.85 (d, J = 8.8 Hz, |
| | | | | | 1H), 6.83 (d, J = 3.9 Hz, 1H), |
| | | | | | 6.10 (d, J = 6.7 Hz, 1H), 3.83 (s, |
| | | | | | 3H), 1.91 (s, 1H). |
| 296 | 280 | 457.54 | N-[(6-aminopyridin- | HPLC | 1H NMR (400 MHz, DMSO) δ |
| | | | 3-yl)(pyridin-3- | 98.5%; | 8.71 (d, J = 2.2 Hz, 1H), 8.60 (d, |
| | | | yl)methyl]-8-(1- | m/z = 458.4 | J = 1.9 Hz, 1H), 8.48 (dd, J = 6.0, |
| | | | methyl-1H-indol-6- | [M + H] | 1.8 Hz, 2H), 8.02 (d, J = 2.4 Hz, |
| | | | yl)quinoxalin-6- | | 1H), 7.85 (dt, J = 8.0, 2.0 Hz, |
| | | | amine | | 1H), 7.59 (m, 3H), 7.43 (d, J = 2.5 Hz, |
| | | | | | 1H), 7.41 (d, J = 2.8 Hz, |
| | | | | | 1H), 7.39 (d, J = 3.5 Hz, 1H), |
| | | | | | 7.38 (s, 1H), 7.27 (dd, J = 8.1, |
| | | | | | 1.5 Hz, 1H), 6.78 (d, J = 2.6 Hz, |
| | | | | | 1H), 6.47 (dd, J = 3.1, 0.8 Hz, |
| | | | | | 1H), 6.45 (d, J = 8.6 Hz, 1H), |
| | | | | | 5.95 (s, 2H), 5.87 (d, J = 6.7 Hz, |
| | | | | | 1H), 3.81 (s, 3H). |
| 297 | 281 | 486.58 | N-[(6- | HPLC 82%; | 1H NMR (400 MHz, DMSO-d6) |
| | | | methoxypyridin-3- | m/z = 487.3 | δ 9.16 (s, 1H), 8.93 (d, J = 6.0 Hz, |
| | | | yl)(pyridin-3- | [M + H] | 1H), 8.66 (d, J = 1.9 Hz, 1H), |
| | | | yl)methyl]-N- | | 8.54 (d, J = 1.9 Hz, 1H), |
| | | | methyl-8-(1-methyl- | | 8.35-8.33 (m, 1H), 8.16 (dd, J = 8.2, |
| | | | 1H-indol-6- | | 6.0 Hz, 1H), 7.84 (dd, J = 8.7, |
| | | | yl)quinoxalin-6- | | 2.5 Hz, 1H), 7.64-7.61 (m, |
| | | | amine | | 2H), 7.60 (s, 1H), 7.40 (d, J = 3.1 Hz, |
| | | | | | 1H), 7.29 (dd, J = 8.0, |
| | | | | | 1.6 Hz, 1H), 6.91 (d, J = 8.6 Hz, |
| | | | | | 1H), 6.85 (d, J = 2.6 Hz, 1H), |
| | | | | | 6.48 (dd, J = 3.1, 0.8 Hz, 1H), |
| | | | | | 6.31 (d, J = 6.6 Hz, 1H), 4.38 (s, |
| | | | | | 3H), 3.86 (s, 3H), 3.82 (s, 3H). |
| 298 | 282 | 505.63 | N-methyl-4-({[8-(1- | HPLC | 1H NMR (400 MHz, DMSO) δ |
| | | | methyl-1H-indol-6- | 96.0%; | 8.71 (m, 1H), 8.57 (d, J = 1.9 Hz, |
| | | | yl)quinoxalin-6- | m/z = 506.4 | 1H), 8.44 (m, 1H), 8.42 (d, J = 1.9 Hz, |
| | | | yl]amino}(pyridin-3- | [M + H] | 1H), 7.86 (dt, J = 8.0, |
| | | | yl)methyl)piperidine- | | 2.0 Hz, 1H), 7.60 (m, 2H), |
| | | | 1- | | 7.51 (d, J = 2.6 Hz, 1H), 7.38 (m, |
| | | | carboxamide | | 2H), 7.25 (m, 2H), 6.70 (d, J = 2.6 Hz, |
| | | | | | 1H), 6.47 (dd, J = 3.1, |
| | | | | | 0.8 Hz, 1H), 6.36 (t, J = 4.4 Hz, |
| | | | | | 1H), 4.54 (t, J = 7.9 Hz, 1H), |
| | | | | | 3.97 (dd, J = 39.3, 13.3 Hz, 3H), |
| | | | | | 3.82 (s, 3H), 2.61 (q, J = 12.3, |
| | | | | | 11.7 Hz, 3H), 2.55 (d, J = 4.3 Hz, |
| | | | | | 3H), 1.96 (d, J = 14.1 Hz, |
| | | | | | 3H). |
| 299 | 283 | 476.54 | N-[(6- | HPLC | 1H NMR (400 MHz, DMSO) δ |
| | | | methoxypyridin-3- | 96.3%; | 8.66 (d, J = 1.9 Hz, 1H), 8.52 (d, |
| | | | yl)(1-methyl-1H- | m/z = 477.4 | J = 1.9 Hz, 1H), 8.39 (d, J = 2.4 Hz, |
| | | | 1,2,3- | [M + H] | 1H), 7.85 (dd, J = 8.6, 2.5 Hz, |
| | | | triazol-5-yl)methyl]- | | 1H), 7.61 (m, 2H), 7.55 (d, J = 7.8 Hz, |
| | | | 8-(1-methyl-1H- | | 1H), 7.52 (d, J = 2.6 Hz, |
| | | | indol-6- | | 1H), 7.39 (m, 2H), 7.27 (dd, |
| | | | yl)quinoxalin-6- | | J = 8.2, 1.5 Hz, 1H), 6.98 (d, J = 2.6 Hz, |
| | | | amine | | 1H), 6.91 (dd, J = 8.6, |
| | | | | | 0.7 Hz, 1H), 6.48 (dd, J = 3.1, |
| | | | | | 0.8 Hz, 1H), 6.34 (d, J = 7.7 Hz, |
| | | | | | 1H), 4.05 (s, 3H), 3.86 (s, 3H), |
| | | | | | 3.82 (s, 3H). |
| 300 | 284 | 519.65 | N,N-dimethyl-4-({[8- | HPLC | 1H NMR (400 MHz, DMSO-d6) |
| | | | (1-methyl-1H-indol- | 95.9%; | δ 8.72 (m, 1H), 8.57 (d, J = 2.0 Hz, |
| | | | 6- | m/z = 520.4 | 1H), 8.44 (dd, J = 4.7, 1.6 Hz, |
| | | | yl)quinoxalin-6- | [M + H] | 1H), 8.42 (d, J = 1.9 Hz, 1H), |
| | | | yl]amino}(pyridin-3- | | 7.87 (dt, J = 7.9, 2.0 Hz, 1H), |
| | | | yl)methyl)piperidine- | | 7.60 (s, 1H), 7.58 (d, J = 0.7 Hz, |
| | | | 1-carboxamide | | 1H), 7.50 (d, J = 2.6 Hz, 1H), |
| | | | | | 7.39 (d, J = 3.0 Hz, 1H), |
| | | | | | 7.36 (m, 1H), 7.29 (s, 1H), 7.24 (d, J = 1.4 Hz, |
| | | | | | 1H), 6.72 (d, J = 2.6 Hz, |
| | | | | | 1H), 6.48 (dd, J = 3.1, 0.8 Hz, |
| | | | | | 1H), 4.55 (t, J = 8.1 Hz, 1H), |
| | | | | | 3.82 (s, 3H), 3.63 (d, J = 12.7 Hz, |
| | | | | | 1H), 3.53 (d, J = 16.0 Hz, |
| | | | | | 1H), 2.72 (s, 6H), 2.67 (s, 2H), |
| | | | | | 2.63 (d, J = 4.5 Hz, 1H), 2.58 (d, |
| | | | | | J = 13.1 Hz, 1H), 2.04 (d, J = 12.9 Hz, |
| | | | | | 1H), 1.91 (s, 1H), |
| | | | | | 1.36 (m, 1H). |
| 301 | 285 | 368.444 | 8-(1-methyl-1H-indol- | HPLC | 1H NMR (400 MHz, DMSO-d6) |
| | | | 6-yl)-N-[(1-methyl-1H- | 96.2%; | δ 8.64 (d, J = 1.9 Hz, 1H), |
| | | | pyrazol-5- | m/z = 369.4 | 8.48 (d, J = 1.9 Hz, 1H), |
| | | | yl)methyl]quinoxalin- | [M + H] | 7.65-7.56 (m, 2H), 7.44 (d, J = 2.6 Hz, |
| | | | 6-amine | | 1H), 7.38 (d, J = 3.1 Hz, 1H), |
| | | | | | 7.34 (d, J = 1.8 Hz, 1H), |
| | | | | | 7.26 (dd, J = 8.2, 1.5 Hz, 1H), |
| | | | | | 7.11 (s, 1H), 6.94 (d, J = 2.6 Hz, 1H), |
| | | | | | 6.47 (d, J = 3.0 Hz, 1H), 6.29 (d, |
| | | | | | J = 1.8 Hz, 1H), 4.53 (d, J = 5.4 Hz, |
| | | | | | 2H), 3.87 (s, 3H), 3.81 (s, |
| | | | | | 3H). |
| 302 | 286 | 493.59 | N-[(6- | HPLC | 1H NMR (400 MHz, DMSO-d6) |
| | | | methoxypyridin-3- | 97.2%; | δ 8.68 (d, J = 1.9 Hz, 1H), |
| | | | yl)(1-methyl-1H- | m/z = 494.3 | 8.53 (d, J = 1.9 Hz, 1H), 8.39 (d, J = 2.5 Hz, |
| | | | 1,2,3- | [M + H] | 1H), 8.05 (dd, J = 8.4, |
| | | | triazol-5-yl)methyl]- | | 0.6 Hz, 1H), 7.91 (m, 1H), |
| | | | 8-(3-methyl-1- | | 7.85 (dd, J = 8.6, 2.5 Hz, 1H), |
| | | | benzothiophen-5- | | 7.61 (m, 2H), 7.55 (d, J = 2.6 Hz, |
| | | | yl)quinoxalin-6- | | 1H), 7.46 (d, J = 1.3 Hz, 1H), |
| | | | amine | | 7.41 (s, 1H), 7.01 (d, J = 2.5 Hz, |
| | | | | | 1H), 6.90 (m, 1H), 6.35 (d, J = 7.7 Hz, |
| | | | | | 1H), 4.05 (s, 3H), |
| | | | | | 3.86 (s, 3H), 2.43 (d, J = 1.2 Hz, 3H). |
| 304 | 288 | 488.64 | 8-(1-methyl-1H- | HPLC | 1H NMR (400 MHz, DMSO-d6) |
| | | | indol-6-yl)-N-({8- | 86.9%; | δ 8.69 (d, J = 2.1 Hz, 1H), |
| | | | methyl-8- | m/z = 489.4 | 8.56 (d, J = 1.9 Hz, 1H), 8.43 (d, J = 1.6 Hz, |
| | | | azabicyclo[3.2.1]octan- | [M + H] | 1H), 8.41 (d, J = 1.9 Hz, |
| | | | 3-yl}(pyridin-3- | | 1H), 7.84 (dt, J = 8.0, 2.0 Hz, |
| | | | yl)methyl)quinoxalin- | | 1H), 7.62-7.60 (m, 1H), |
| | | | 6-amine | | 7.58 (s, 1H), 7.48 (d, J = 2.6 Hz, 1H), |
| | | | | | 7.39 (d, J = 3.1 Hz, 1H), |
| | | | | | 7.35 (dd, J = 7.9, 4.8 Hz, 1H), |
| | | | | | 7.25 (dd, J = 8.2, 1.4 Hz, 1H), |
| | | | | | 7.19 (d, J = 7.9 Hz, 1H), 6.67 (d, J = 2.5 Hz, |
| | | | | | 1H), 6.47 (d, J = 3.1 Hz, |
| | | | | | 1H), 3.82 (s, 3H), 3.11 (s, 1H), |
| | | | | | 3.02 (d, J = 5.2 Hz, 1H), 2.14 (s, |
| | | | | | 3H), 2.09-1.80 (m, 10H). |
| 305 | 289 | 471.57 | 8-(1-methyl-1H- | HPLC | 1H NMR (400 MHz, DMSO-d6) |
| | | | indol-6-yl)-N-{[6- | 90.7%; | δ 8.87 (d, J = 1.8 Hz, 1H), |
| | | | (methylamino)pyridin- | m/z = 472.5 | 8.79 (d, J = 1.8 Hz, 1H), 8.63 (d, J = 2.3 Hz, |
| | | | 3-yl](pyridin-3- | [M + H] | 1H), 8.41 (dd, J = 4.8, |
| | | | yl)methyl}quinoxalin- | | 1.7 Hz, 1H), 8.32 (d, J = 2.4 Hz, |
| | | | 6-amine | | 1H), 7.83-7.76 (m, 2H), |
| | | | | | 7.72 (d, J = 2.6 Hz, 1H), |
| | | | | | 7.69-7.58 (m, 3H), 7.39 (d, J = 3.0 Hz, |
| | | | | | 1H), 7.32 (ddd, J = 8.2, 4.7, 1.1 Hz, |
| | | | | | 2H), 7.10 (d, J = 8.6 Hz, 1H), |
| | | | | | 6.47 (dd, J = 3.0, 0.8 Hz, 1H), |
| | | | | | 5.14 (s, 1H), 3.80 (s, 3H), |
| | | | | | 3.62 (s, 3H), 2.44 (s, 2H). |
| 306 | 290 | 368.44 | 8-(1-methyl-1H- | HPLC | 1H NMR (400 MHz, DMSO-d6) |
| | | | indol-6-yl)-N-[(1- | 94.5%; | δ 8.62 (d, J = 1.7 Hz, 1H), |
| | | | methyl-1H-pyrazol-4- | m/z = 369.4 | 8.45 (d, J = 1.7 Hz, 1H), 7.71 (s, 1H), |
| | | | yl)methyl]quinoxalin- | [M + H] | 7.65-7.57 (m, 2H), 7.47 (s, |
| | | | 6-amine | | 1H), 7.39 (dd, J = 10.1, 2.7 Hz, |
| | | | | | 2H), 7.30-7.20 (m, 1H), |
| | | | | | 6.92 (t, J = 5.4 Hz, 1H), 6.86 (d, J = 2.4 Hz, |
| | | | | | 1H), 6.47 (d, J = 2.9 Hz, |
| | | | | | 1H), 4.28 (d, J = 5.3 Hz, 2H), |
| | | | | | 3.81 (s, 6H). |
| 307 | 291 | 499.58 | N-[5-({[8-(1-methyl- | HPLC | not determined |
| | | | 1H-indol-6- | 90.9%; | |
| | | | yl)quinoxalin-6- | m/z = 498.7 | |
| | | | yl]amino}(pyridin-3- | [M + H] | |
| | | | yl)methyl)pyridin-2- | | |
| | | | yl]acetamide | | |
| 308 | 292 | 462.60 | N-[(4- | HPLC | 1H NMR (400 MHz, DMSO-d6) |
| | | | aminocyclohexyl)(pyridin- | 98.0%; | δ 8.69 (d, J = 2.2 Hz, |
| | | | 3-yl)methyl]-8- | m/z = 463.4 | 1H), 8.56 (d, J = 1.9 Hz, 1H), |
| | | | (1- | [M + H] | 8.43 (dd, J = 4.8, 1.7 Hz, 1H), 8.41 (d, J = 1.9 Hz, |
| | | | methyl-1H-indol-6- | | 1H), 7.83 (dt, J = 7.9, 2.0 Hz, 1H), |
| | | | yl)quinoxalin-6- | | 7.60 (m, 2H), 7.50 (t, J = 3.0 Hz, 1H), |
| | | | amine | | 7.38 (m, 2H), 7.25 (dd, J = 8.3, 1.4 Hz, |
| | | | | | 1H), 7.22 (d, J = 7.8 Hz, 1H), |
| | | | | | 6.66 (d, J = 2.6 Hz, 1H), 6.47 (dd, J = 3.0, 0.8 Hz, |
| | | | | | 1H), 4.43 (t, J = 7.7 Hz, 1H), |
| | | | | | 3.82 (s, 3H), 2.08 (d, J = 12.1 Hz, 1H), |
| | | | | | 1.83 (d, J = 13.1 Hz, 1H), |
| | | | | | 1.75 (d, J = 12.3 Hz, 1H), 1.69 (d, J = 8.5 Hz, |
| | | | | | 1H), 1.51 (s, 1H), 1.34 (d, J = 12.0 Hz, |
| | | | | | 1H), 1.25 (d, J = 10.2 Hz, 1H), |
| | | | | | 1.16 (t, J = 11.5 Hz, 2H), 2H) |
| | | | | | 1.12-1.03 (m, 1H) |
| 309 | 293 | 502.58 | N-[bis(6- | HPLC | 1H NMR (400 MHz, DMSO-d6) |
| | | | methoxypyridin-3- | 94.3%; | δ 8.62 (d, J = 1.9 Hz, 1H), |
| | | | yl)methyl]-8-(1- | m/z = 503.4 | 8.49 (d, J = 1.9 Hz, 1H), 8.31 (d, J = 2.4 Hz, |
| | | | methyl- | [M + H] | 2H), 7.77 (dd, J = 8.6, |
| | | | 1H-indol-6- | | 2.5 Hz, 2H), 7.64-7.56 (m, |
| | | | yl)quinoxalin-6- | | 3H), 7.44 (d, J = 6.9 Hz, 1H), |
| | | | amine | | 7.39 (d, J = 3.1 Hz, 1H), |
| | | | | | 7.28 (dd, J = 8.1, 1.5 Hz, 1H), |
| | | | | | 6.85 (dd, J = 8.6, 0.7 Hz, 2H), |
| | | | | | 6.82 (d, J = 2.6 Hz, 1H), 6.48 (dd, J = 3.0, |
| | | | | | 0.8 Hz, 1H), 6.00 (d, J = 6.8 Hz, |
| | | | | | 1H), 3.84 (s, 6H), 3.82 (s, |
| | | | | | 3H) |
| 310 | 294 | 507.66 | 1-{4-[(R)-{[8-(3- | HPLC | 1H NMR (400 MHz, DMSO-d6) |
| | | | methyl-1- | 97.0%; | δ 8.72 (d, J = 2.2 Hz, 1H), |
| | | | benzothiophen-5- | m/z = 508.4 | 8.61-8.58 (m, 1H), 8.45 (dd, J = 4.8, |
| | | | yl)quinoxalin-6- | [M + H] | 1.5 Hz, 1H), 8.43 (d, J = 1.9 Hz, |
| | | | yl]amino}(pyridin-3- | | 1H), 8.03 (d, J = 8.3 Hz, 1H), |
| | | | yl)methyl]piperidin- | | 7.90 (d, J = 1.6 Hz, 1H), 7.87 (d, |
| | | | 1-yl}ethan-1-one | | J = 7.7 Hz, 1H), 7.59 (dd, J = 8.3, |
| | | | | | 1.6 Hz, 1H), |
| | | | | | 7.57-7.53 (m, 1H), 7.46 (d, J = 1.4 Hz, |
| | | | | | 1H), 7.38 (dd, J = 7.8, 4.8 Hz, |
| | | | | | 1H), 7.30 (d, J = 7.9 Hz, 1H), |
| | | | | | 6.74 (s, 1H), 4.58 (q, J = 7.3 Hz, |
| | | | | | 1H), 4.49-4.37 (m, 1H), |
| | | | | | 3.86 (dd, J = 29.5, 13.7 Hz, 1H), |
| | | | | | 3.03-2.92 (m, 1H), 2.44 (d, J = 1.2 Hz, |
| | | | | | 3H), 2.04 (d, J = 22.6 Hz, |
| | | | | | 2H), 1.98 (d, J = 6.2 Hz, 3H), |
| | | | | | 1.32 (d, J = 17.8 Hz, 4H) |
| 311 | 295 | 507.66 | 1-{4-[(S)-{[8-(3- | HPLC | 1H NMR (400 MHz, DMSO-d6) |
| | | | methyl-1- | 94.9%; | δ 8.72 (d, J = 2.2 Hz, 1H), |
| | | | benzothiophen-5- | m/z = 508.4 | 8.61-8.58 (m, 1H), 8.45 (dd, J = 4.8, |
| | | | yl)quinoxalin-6- | [M + H] | 1.6 Hz, 1H), 8.43 (d, J = 1.9 Hz, |
| | | | yl]amino}(pyridin-3- | | 1H), 8.03 (d, J = 8.3 Hz, 1H), |
| | | | yl)methyl]piperidin- | | 7.90 (d, J = 1.6 Hz, 1H), |
| | | | 1-yl}ethan-1-one | | 7.86 (dd, J = 7.9, 2.1 Hz, 1H), |
| | | | | | 7.59 (dd, J = 8.3, 1.6 Hz, 1H), 7.55 (t, |
| | | | | | J = 2.5 Hz, 1H), 7.46 (d, J = 1.4 Hz, |
| | | | | | 1H), 7.38 (dd, J = 7.8, 4.7 Hz, |
| | | | | | 1H), 7.30 (d, J = 7.9 Hz, 1H), |
| | | | | | 6.74 (s, 1H), 4.58 (q, J = 7.2 Hz, |
| | | | | | 1H), 4.48-4.36 (m, 1H), |
| | | | | | 3.85 (dd, J = 17.8, 11.2 Hz, 1H), |
| | | | | | 3.03-2.93 (m, 1H), 2.44 (d, J = 1.2 Hz, |
| | | | | | 3H), 2.04 (d, J = 22.8 Hz, |
| | | | | | 2H), 1.98 (d, J = 6.2 Hz, 3H), |
| | | | | | 1.44-1.28 (m, 4H). |
| 312 | 296 | 369.43 | N-[(2-methyl-1,3- | HPLC | 1H NMR (400 MHz, DMSO-d6) |
| | | | oxazol-5-yl)methyl]- | 99.5%; | δ 8.66 (d, J = 1.9 Hz, 1H), |
| | | | 8-(1-methyl- | m/z = 370.3 | 8.49 (d, J = 1.9 Hz, 1H), |
| | | | 1H-indol-6- | [M + H] | 7.63-7.62 (m, 1H), 7.59 (d, J = 0.7 Hz, |
| | | | yl)quinoxalin-6- | | 1H), 7.41 (d, J = 2.6 Hz, 1H), |
| | | | amine | | 7.39 (d, J = 3.1 Hz, 1H), |
| | | | | | 7.26 (dd, J = 8.1, 1.5 Hz, 1H), 7.14 (t, |
| | | | | | J = 5.8 Hz, 1H), 7.03 (s, 1H), |
| | | | | | 6.96 (d, J = 2.6 Hz, 1H), |
| | | | | | 6.48 (dd, J = 3.0, 0.9 Hz, 1H), |
| | | | | | 4.50 (d, J = 5.7 Hz, 2H), 3.82 (s, 3H), |
| | | | | | 2.39 (s, 3H). |
| 313 | 297 | 462.58 | 8-(3-methyl-1- | HPLC | 1H NMR (400 MHz, DMSO-d6) |
| | | | benzothiophen-5-yl)- | 93.4%; | δ 8.81 (d, J = 2.2 Hz, 1H), |
| | | | N-[(1-methyl-1H- | m/z = 463.3 | 8.64 (d, J = 2.0 Hz, 1H), 8.55 (dd, J = 4.8, |
| | | | imidazol-5- | [M + H] | 1.6 Hz, 1H), 8.49 (d, J = 1.9 Hz, |
| | | | yl)(pyridin-3- | | 1H), 8.04 (d, J = 8.3 Hz, 1H), |
| | | | yl)methyl]quinoxalin- | | 7.97 (dt, J = 8.0, 2.0 Hz, 1H), |
| | | | 6- | | 7.90 (d, J = 1.6 Hz, 1H), 7.70 (d, |
| | | | amine | | J = 1.1 Hz, 1H), 7.62-7.59 (m, |
| | | | | | 1H), 7.58 (d, J = 2.6 Hz, 1H), |
| | | | | | 7.56 (s, 1H), 7.47 (d, J = 4.2 Hz, |
| | | | | | 1H), 7.45 (d, J = 4.9 Hz, 1H), |
| | | | | | 6.95 (d, J = 2.6 Hz, 1H), 6.33 (s, |
| | | | | | 1H), 6.20 (d, J = 7.5 Hz, 1H), |
| | | | | | 3.68 (s, 3H), 1.91 (s, 3H). |
| 314 | 298 | 475.56 | N-[(6- | HPLC | 1H NMR (400 MHz, DMSO-d6) |
| | | | methoxypyridin-3- | 99.3%; | δ 8.62 (d, J = 1.9 Hz, 1H), |
| | | | yl)(1-methyl-1H- | m/z = 476.3 | 8.48 (d, J = 1.9 Hz, 1H), 8.38 (d, J = 2.4 Hz, |
| | | | imidazol-5- | [M + H] | 1H), 7.87 (dd, J = 8.6, |
| | | | yl)methyl]-8-(1- | | 2.5 Hz, 1H), 7.68 (d, J = 1.1 Hz, |
| | | | methyl-1H-indol-6- | | 1H), 7.60 (dd, J = 4.7, 0.8 Hz, |
| | | | yl)quinoxalin-6- | | 1H), 7.59 (s, 1H), 7.46 (d, J = 7.6 Hz, |
| | | | amine | | 1H), 7.39 (d, J = 3.0 Hz, |
| | | | | | 1H), 7.26 (dd, J = 8.3, 1.4 Hz, |
| | | | | | 1H), 6.92 (d, J = 2.6 Hz, 1H), |
| | | | | | 6.88 (dd, J = 8.5, 0.7 Hz, 1H), |
| | | | | | 6.47 (dd, J = 3.1, 0.8 Hz, 1H), |
| | | | | | 6.39 (d, J = 1.0 Hz, 1H), 6.09 (d, |
| | | | | | J = 7.6 Hz, 1H), 3.85 (s, 3H), |
| | | | | | 3.81 (s, 3H), 3.66 (s, 3H), |
| | | | | | 3.17 (d, J = 5.2 Hz, 1H). |
| 315 | 299 | 492.60 | N-[(6- | HPLC | 1H NMR (400 MHz, DMSO-d6) |
| | | | methoxypyridin-3- | 97.1%; | δ 8.65 (d, J = 1.9 Hz, 1H), |
| | | | yl)(1-methyl-1H- | m/z = 493.3 | 8.49 (d, J = 1.9 Hz, 1H), 8.38 (d, J = 2.4 Hz, |
| | | | imidazol-5- | [M + H] | 1H), 8.04 (dd, J = 8.3, |
| | | | yl)methyl]-8-(3- | | 0.6 Hz, 1H), 7.90 (d, J = 1.6 Hz, |
| | | | methyl-1- | | 1H), 7.87 (dd, J = 8.6, 2.5 Hz, |
| | | | benzothiophen-5- | | 1H), 7.68 (d, J = 1.0 Hz, 1H), |
| | | | yl)quinoxalin-6- | | 7.60 (dd, J = 8.2, 1.6 Hz, 1H), |
| | | | amine | | 7.56 (d, J = 2.6 Hz, 1H), 7.49 (d, |
| | | | | | J = 7.6 Hz, 1H), 7.46 (d, J = 1.3 Hz, |
| | | | | | 1H), 6.96 (d, J = 2.5 Hz, 1H), |
| | | | | | 6.88 (m, 1H), 6.39 (d, J = 0.9 Hz, |
| | | | | | 1H), 6.10 (d, J = 7.5 Hz, 1H), |
| | | | | | 3.85 (s, 3H), 3.66 (s, 3H), |
| | | | | | 2.43 (d, J = 1.2 Hz, 3H). |
| 316 | 300 | 477.53 | N-[(6- | HPLC | 1H NMR (400 MHz, DMSO-d6) |
| | | | methoxypyridin-3- | 96.8%; | δ 8.67 (d, J = 1.9 Hz, 1H), |
| | | | yl)(1-methyl-1H- | m/z = 478.1 | 8.53 (d, J = 1.9 Hz, 1H), 8.39 (d, J = 2.4 Hz, |
| | | | 1,2,3- | [M + H] | 1H), 7.86-7.82 (m, |
| | | | triazol-5-yl)methyl]- | | 2H), 7.76 (d, J = 1.7 Hz, 1H), |
| | | | 8-(3-methyl-1- | | 7.62 (dd, J = 8.5, 0.6 Hz, 1H), |
| | | | benzofuran-5- | | 7.57-7.50 (m, 3H), 7.40 (s, |
| | | | yl)quinoxalin-6- | | 1H), 6.99 (d, J = 2.6 Hz, 1H), |
| | | | amine | | 6.93-6.88 (m, 1H), 6.34 (d, J = 7.7 Hz, |
| | | | | | 1H), 4.04 (s, 3H), |
| | | | | | 3.86 (s, 3H), 2.24 (d, J = 1.3 Hz, 3H). |
| 317 | 301 | 475.56 | N-[(6- | HPLC | 1H NMR (400 MHz, DMSO-d6) |
| | | | methoxypyridin-3- | 97.2%; | δ 8.64 (d, J = 1.9 Hz, 1H), |
| | | | yl)(1-methyl-1H- | m/z = 476.1 | 8.49 (d, J = 1.9 Hz, 1H), 8.37 (d, J = 2.5 Hz, |
| | | | pyrazol-5- | [M + H] | 1H), 7.83 (dd, J = 8.6, |
| | | | yl)methyl]-8-(1- | | 2.5 Hz, 1H), 7.62-7.59 (m, |
| | | | methyl-1H-indol-6- | | 2H), 7.55 (d, J = 2.6 Hz, 1H), |
| | | | yl)quinoxalin-6- | | 7.48 (d, J = 7.6 Hz, 1H), 7.39 (d, |
| | | | amine | | J = 3.0 Hz, 1H), 7.35 (d, J = 1.9 Hz, |
| | | | | | 1H), 7.27 (dd, J = 8.2, 1.4 Hz, |
| | | | | | 1H), 6.95 (d, J = 2.5 Hz, 1H), |
| | | | | | 6.88 (dd, J = 8.5, 0.7 Hz, 1H), |
| | | | | | 6.48 (dd, J = 3.1, 0.8 Hz, 1H), |
| | | | | | 6.20 (d, J = 7.6 Hz, 1H), |
| | | | | | 5.94 (dd, J = 1.8, 0.5 Hz, 1H), |
| | | | | | 3.86 (s, 3H), 3.85 (s, 3H), 3.82 (s, |
| | | | | | 3H). |
| 318 | 302 | 526.66 | N-[(1-methanesulfonylpiperidin- | HPLC | 1H NMR (400 MHz, DMSO-d6) |
| | | | 4-yl)(pyridin- | 81.3%; | δ 8.73 (s, 1H), 8.64-8.55 (m, |
| | | | 3- | m/z = 527.7 | 1H), 8.44 (dd, J = 11.4, 3.1 Hz, |
| | | | yl)methyl]-8-(1-methyl- | [M + H] | 2H), 7.94-7.68 (m, 2H), |
| | | | 1H-indol-6-yl)quinoxalin- | | 7.68-7.34 (m, 4H), 7.34-7.14 (m, |
| | | | 6- | | 2H), 6.91-6.66 (m, 2H), |
| | | | amine | | 4.78-4.53 (m, 1H), 3.92 (d, J = 31.0 Hz, |
| | | | | | 1H), 3.83 (d, J = 3.4 Hz, 1H), |
| | | | | | 3.61 (dd, J = 36.1, 11.3 Hz, 2H), |
| | | | | | 2.86 (d, J = 2.6 Hz, 3H), |
| | | | | | 2.75-2.60 (m, 3H), 2.11 (m, 1H), |
| | | | | | 1.90 (m, 1H), 1.45 (m, J = 31.6 Hz, |
| | | | | | 3H), 1.20 (d, J = 35.5 Hz, |
| | | | | | 1H). |
| 319 | 303 | 478.57 | N-[(6- | HPLC | 1H NMR (400 MHz, DMSO-d6) |
| | | | methoxypyridin-3- | 92.4%; | δ 8.66 (d, J = 1.9 Hz, 1H), |
| | | | yl)(1,2-thiazol-5- | m/z = 479.1 | 8.52 (dd, J = 4.3, 1.8 Hz, 2H), |
| | | | yl)methyl]-8-(1- | [M + H] | 8.43 (d, J = 2.4 Hz, 1H), 7.88 (dd, J = 8.6, |
| | | | methyl-1H-indol-6- | | 2.5 Hz, 1H), 7.71 (d, J = 7.8 Hz, |
| | | | yl)quinoxalin-6- | | 1H), 7.64-7.59 (m, 2H), |
| | | | amine | | 7.56 (d, J = 2.6 Hz, 1H), 7.39 (d, |
| | | | | | J = 3.0 Hz, 1H), 7.32 (dd, J = 1.7, |
| | | | | | 0.9 Hz, 1H), 7.27 (dd, J = 8.2, |
| | | | | | 1.5 Hz, 1H), 7.02 (d, J = 2.6 Hz, |
| | | | | | 1H), 6.89 (dd, J = 8.6, 0.7 Hz, |
| | | | | | 1H), 6.54 (d, J = 7.8 Hz, 1H), |
| | | | | | 6.48 (dd, J = 3.0, 0.9 Hz, 1H), |
| | | | | | 3.85 (s, 3H), 3.82 (s, 3H). |
| 320 | 304 | 456.553 | N-[1,1-bis(pyridin-3- | HPLC | not determined |
| | | | yl)ethyl]-8-(1- | 97.3%; | |
| | | | methyl-1H- | m/z = 457.2 | |
| | | | indol-6-yl)quinoxalin- | [M + H] | |
| | | | 6-amine | | |
| 321 | 305 | 395.466 | N-[(6- | HPLC | 1H NMR (400 MHz, DMSO-d6) |
| | | | methoxypyridin-3- | 99.1%; | δ 8.62 (d, J = 1.9 Hz, 1H), |
| | | | yl)methyl]-8-(1- | m/z = 396.3 | 8.46 (d, J = 1.9 Hz, 1H), |
| | | | methyl-1H- | [M + H] | 8.29-8.26 (m, 1H), 7.78 (dd, J = 8.5, 2.5 Hz, |
| | | | indol-6-yl)quinoxalin- | | 1H), 7.62-7.59 (m, 2H), |
| | | | 6-amine | | 7.42 (d, J = 2.6 Hz, 1H), 7.39 (d, |
| | | | | | J = 3.1 Hz, 1H), 7.26 (dd, J = 8.1, |
| | | | | | 1.5 Hz, 1H), 7.21 (t, J = 5.8 Hz, |
| | | | | | 1H), 6.85-6.81 (m, 2H), |
| | | | | | 6.48 (dd, J = 3.1, 0.8 Hz, 1H), |
| | | | | | 4.43 (d, J = 5.7 Hz, 2H), 3.84 (s, |
| | | | | | 3H), 3.82 (s, 3H). |
| 322 | 306 | 366.428 | 8-(1-methyl-1H- | HPLC | 1H NMR (400 MHz, DMSO) δ |
| | | | indol-6-yl)-N- | 87.0%; | 9.33 (s, 1H), 9.18 (dd, J = 5.3, |
| | | | (pyridazin-4- | m/z = 367.2 | 1.2 Hz, 1H), 8.62 (d, J = 1.9 Hz, |
| | | | ylmethyl)quinoxalin- | [M + H] | 1H), 8.49 (d, J = 1.9 Hz, 1H), |
| | | | 6-amine | | 7.69 (dd, J = 5.3, 2.3 Hz, 1H), |
| | | | | | 7.63 (s, 1H), 7.61 (d, J = 8.2 Hz, |
| | | | | | 1H), 7.46 (d, J = 2.6 Hz, 1H), |
| | | | | | 7.42-7.37 (m, 2H), 7.28 (dd, J = 8.1, |
| | | | | | 1.4 Hz, 1H), 6.75 (d, J = 2.6 Hz, |
| | | | | | 1H), 6.50-6.47 (m, |
| | | | | | 1H), 4.64 (d, J = 6.1 Hz, 2H), |
| | | | | | 3.82 (s, 3H). |
| 323 | 307 | 493.59 | N-[(R)-(6- | HPLC | ¹H NMR (500 MHz, DMSO-d₆) δ |
| | | | methoxypyridin-3- | 99%; | 8.67 (d, J = 1.9 Hz, 1H), 8.52 (d, |
| | | | yl)(1-methyl-1H- | m/z = 494.2 | J = 1.9 Hz, 1H), 8.38 (d, J = 2.4 Hz, |
| | | | 1,2,3- | [M + H] | 1H), 8.03 (d, J = 8.3 Hz, 1H), |
| | | | triazol-5-yl)methyl]- | | 7.92-7.90 (m, 1H), 7.84 (dd, J = 8.6, |
| | | | 8-(3-methyl-1- | | 2.5 Hz, 1H), 7.61 (dd, J = 8.3, |
| | | | benzothiophen-5- | | 1.7 Hz, 1H), 7.57-7.54 (m, |
| | | | yl)quinoxalin-6- | | 2H), 7.46-7.44 (m, 1H), 7.40 (s, |
| | | | amine | | 1H), 7.01 (d, J = 2.6 Hz, 1H), |
| | | | | | 6.89 (d, J = 8.6 Hz, 1H), 6.34 (d, |
| | | | | | J = 7.7 Hz, 1H), 4.04 (s, 3H), |
| | | | | | 3.85 (s, 3H), 2.42 (d, J = 1.3 Hz, |
| | | | | | 3H). |
| 324 | 308 | 493.59 | N-[(S)-(6- | HPLC | ¹H NMR (500 MHz, DMSO-d₆) δ |
| | | | methoxypyridin-3- | 99.5%; | 8.67 (d, J = 1.9 Hz, 1H), 8.52 (d, |
| | | | yl)(1-methyl-1H- | m/z = 494.2 | J = 1.9 Hz, 1H), 8.38 (d, J = 2.4 Hz, |
| | | | 1,2,3- | [M + H] | 1H), 8.03 (d, J = 8.3 Hz, 1H), |
| | | | triazol-5-yl)methyl]- | | 7.91-7.90 (m, 1H), 7.84 (dd, J = 8.6, |
| | | | 8-(3-methyl-1- | | 2.5 Hz, 1H), 7.61 (dd, J = 8.3, |
| | | | benzothiophen-5- | | 1.6 Hz, 1H), 7.57-7.54 (m, |
| | | | yl)quinoxalin-6- | | 2H), 7.46-7.44 (m, 1H), 7.40 (s, |
| | | | amine | | 1H), 7.01 (d, J = 2.5 Hz, 1H), |
| | | | | | 6.90 (d, J = 8.8 Hz, 1H), 6.34 (d, |
| | | | | | J = 7.7 Hz, 1H), 4.04 (s, 3H), |
| | | | | | 3.85 (s, 3H), 2.43 (d, J = 1.1 Hz, |
| | | | | | 3H) |
| 325 | 309 | 476.544 | N-[(R)-(6- | HPLC | ¹H NMR (500 MHz, DMSO-d₆) δ |
| | | | methoxypyridin-3- | 99.5%; | 8.65 (d, J = 1.9 Hz, 1H), 8.51 (d, |
| | | | yl)(1-methyl-1H- | m/z = 477.2 | J = 1.9 Hz, 1H), 8.38 (d, J = 2.4 Hz, |
| | | | 1,2,3- | [M + H] | 1H), 7.84 (dd, J = 8.6, 2.5 Hz, |
| | | | triazol-5-yl)methyl]- | | 1H), 7.62-7.58 (m, 2H), |
| | | | 8-(1-methyl-1H- | | 7.53-7.50 (m, 2H), |
| | | | indol-6- | | 7.40-7.39 (m, 1H), 7.38 (d, J = 3.1 Hz, |
| | | | yl)quinoxalin-6- | | 1H), 7.27 (dd, J = 8.0, 1.5 Hz, |
| | | | amine | | 1H), 6.97 (d, J = 2.6 Hz, 1H), |
| | | | | | 6.89 (d, J = 8.6 Hz, 1H), |
| | | | | | 6.48-6.46 (m, 1H), 6.32 (d, J = 7.7 Hz, |
| | | | | | 1H), 4.04 (s, 3H), 3.85 (s, |
| | | | | | 3H), 3.81 (s, 3H) |
| 326 | 310 | 476.544 | N-[(S)-(6- | HPLC 99%; | ¹H NMR (500 MHz, DMSO-d₆) δ |
| | | | methoxypyridin-3- | m/z = 477.2 | 8.65 (d, J = 1.9 Hz, 1H), 8.51 (d, |
| | | | yl)(1-methyl-1H- | [M + H] | J = 1.8 Hz, 1H), 8.38 (d, J = 2.4 Hz, |
| | | | 1,2,3- | | 1H), 7.84 (dd, J = 8.6, 2.5 Hz, |
| | | | triazol-5-yl)methyl]- | | 1H), 7.61-7.58 (m, 2H), |
| | | | 8-(1-methyl-1H- | | 7.53-7.50 (m, 2H), |
| | | | indol-6- | | 7.40-7.39 (m, 1H), 7.38 (d, J = 3.1 Hz, |
| | | | yl)quinoxalin-6- | | 1H), 7.27 (dd, J = 8.1, 1.6 Hz, |
| | | | amine | | 1H), 6.97 (d, J = 2.6 Hz, 1H), |
| | | | | | 6.89 (d, J = 8.6 Hz, 1H), |
| | | | | | 6.48-6.46 (m, 1H), 6.32 (d, J = 7.7 Hz, |
| | | | | | 1H), 4.04 (s, 3H), 3.85 (s, |
| | | | | | 3H), 3.81 (s, 3H) |
| 327 | 311 | 504.638 | N-[(1R,4r)-4-[(R)-{[8- | HPLC | ¹H NMR (500 MHz, DMSO-d₆) δ |
| | | | (1-methyl-1H-indol- | 100%; | 8.69 (d, J = 2.3 Hz, 1H), 8.55 (d, |
| | | | 6- | m/z = 253.2 | J = 1.9 Hz, 1H), 8.43 (dd, J = 4.7, |
| | | | yl)quinoxalin-6- | [M + H] | 1.6 Hz, 1H), 8.41 (d, J = 1.9 Hz, |
| | | | yl]amino}(pyridin-3- | | 1H), 7.85-7.82 (m, 1H), |
| | | | yl)methyl]cyclohexyl]acetamide | | 7.67 (d, J = 7.8 Hz, 1H), |
| | | | | | 7.61-7.57 (m, 2H), 7.50 (d, J = 2.6 Hz, |
| | | | | | 1H), 7.39-7.34 (m, 2H), |
| | | | | | 7.25 (dd, J = 8.0, 1.5 Hz, 1H), |
| | | | | | 7.17 (d, J = 7.6 Hz, 1H), 6.65 (d, |
| | | | | | J = 2.5 Hz, 1H), 6.48-6.46 (m, |
| | | | | | 1H), 4.46 (t, J = 7.6 Hz, 1H), |
| | | | | | 3.81 (s, 3H), 3.52-3.44 (m, 1H), |
| | | | | | 2.11-2.04 (m, 1H), |
| | | | | | 1.89-1.83 (m, 1H), 1.80-1.68 (m, 5H), |
| | | | | | 1.43-1.37 (m, 1H), |
| | | | | | 1.30-1.03 (m, 4H) |
| 328 | 312 | 504.638 | N-[(1S,4r)-4-[(S)-{[8- | HPLC 99%; | ¹H NMR (500 MHz, DMSO-d₆) δ |
| | | | (1-methyl-1H-indol- | m/z = 253.2 | 8.69 (d, J = 2.1 Hz, 1H), 8.55 (d, |
| | | | 6- | [M + H] | J = 1.9 Hz, 1H), 8.43 (dd, J = 4.7, |
| | | | yl)quinoxalin-6- | | 1.6 Hz, 1H), 8.41 (d, J = 1.9 Hz, |
| | | | yl]amino}(pyridin-3- | | 1H), 7.85-7.82 (m, 1H), |
| | | | yl)methyl]cyclohexyl]acetamide | | 7.67 (d, J = 7.8 Hz, 1H), |
| | | | | | 7.61-7.57 (m, 2H), 7.50 (d, J = 2.6 Hz, |
| | | | | | 1H), 7.38-7.34 (m, 2H), |
| | | | | | 7.25 (dd, J = 8.1, 1.5 Hz, 1H), |
| | | | | | 7.17 (d, J = 7.7 Hz, 1H), 6.65 (d, |
| | | | | | J = 2.5 Hz, 1H), 6.48-6.46 (m, |
| | | | | | 1H), 4.46 (t, J = 7.6 Hz, 1H), |
| | | | | | 3.81 (s, 3H), 3.52-3.44 (m, 1H), |
| | | | | | 2.11-2.04 (m, 1H), |
| | | | | | 1.89-1.83 (m, 1H), 1.80-1.68 (m, 5H), |
| | | | | | 1.43-1.36 (m, 1H), |
| | | | | | 1.29-1.04 (m, 4H) |

### Biological Activity

Biological activity of the compounds of the present invention is determined utilizing the assays described hereinbelow.

### PFKFB3 IC50 Determination Assay

In vitro kinase assay used to determine IC₅₀ values for tested inhibitors is based on a modified ADP-Glo™ system (Promega) and consists of two parts:

1. Kinase reaction—performed in optimized conditions. At this step PFKFB3 phosphorylates its substrate fructose-6-phosphate using ATP as a source of phosphate to produce fructose-2,6-bisphosphate and ADP. Reaction is performed at Km values for ATP and substrate using optimized buffer composition and time of the reaction. Human recombinant His-tagged PFKFB3 (PFKFB3 BATCH II SEC) with confirmed activity is produced and purified in-house.

2. Detection of ADP as a product of the reaction using ADP-Glo™ system. This part is conducted by using the commercially available kit ADP-Glo™ Kinase Assay (Promega, cat. No# V9103) according to manufacturer's instruction, modified by 5× dilutions of assay reagents (both ADP-Glo™ Reagent and Kinase Detection Solution). Reproducibility and reliability of this modification is confirmed in an optimization process.

Test compounds are dissolved in DMSO and then transferred to the V-bottom 96-well plate. For IC50 determination ten 10× serial dilutions starting from 100 μM are prepared.

Two mixes are prepared on ice: Mix 1—containing appropriate kinase amount in 2× reaction buffer (100 mM TRIS pH 8.0) and Mix 2—containing 2.31× concentrated substrate (Fructose-6-phosphate) and ATP in MilliQ water. 15 μL per well of Mix 1 is transferred to assay wells of 96-well white plate. Next, 2 μl of 15× concentrated test compound in DMSO is added to Mix 1 for 20 min pre-incubation, followed by addition of Mix 2 (13 μl/well). Total reaction volume is 30 μL per well. Samples are tested in duplicates. Final concentration of DMSO in the reaction is 6.7%. Conditions needed for performing PFKFB3 (PFKFB3 BATCH II SEC) in vitro kinase assay are given below:

| | Final concentration/ | |
|---|---|---|
| Reagent/condition | final condition | |
| Buffer | 100 mM Tris, pH 8.0 | |
| MgCl₂ | 5 | mM |
| KF | 20 | mM |
| DTT | 1 | mM |
| KH₂PO₄ | 5 | mM |
| BSA | 0.02% | |
| Tween-20 | 0.005% | |
| ATP (Km) | 20 | μM |
| (ultrapure, from | | |
| ADP-Glo 1™ kit) | | |
| Substrate Fructose- | 2 | μM |
| 6-phosphate (Km) | | |
| Sigma cat no. | | |
| F3627 | | |
| In-house produced | 25 | nM |
| human recombinant | | |
| PFKFB3 (PFKFB3 | | |
| BATCH II SEC) | | |
| Time of reaction | 2 | h |
| Temperature of | rt | |
| reaction | | |

This protocol is based on Technical Bulletin, ADP-Glo™ Kinase Assay (Promega) and is adapted to 96-well plate containing 30 μL reaction mixture:

30 μL of 5× diluted ADP-Glo™ Reagent is added to each well of 96-well plate containing 30 μL of reaction mixture. The plate is incubated for 90 minutes on a shaker at rt. 60 μL of 5× diluted Kinase Detection Solution is added to each well of 96-well plate containing 60 μL of the solution (ratio of kinase reaction volume to ADP-Glo™ Reagent volume to Kinase Detection Solution volume is maintained at 1:1:2). Plate is incubated for 40 minutes on a shaker at rt, protected from light. Luminescence is measured in the plate reader Synergy 2 (BioTek).

Luminescent readouts for compounds tested in 10 concentrations (routinely from 100 μM to 1 nM, 10-fold serial dilutions) in duplicates, as well as for positive control, are first normalized to no-substrate negative control by its subtraction. In the next step, % of normalized positive control is calculated for each data point and plotted against test compound concentration:

$$\% of control = 100 \% \times \frac{( {Lum}_{cpd} - {Lum}_{neg} )}{( {Lum}_{pos} - {Lum}_{neg} )}$$

% of control—percent of positive control normalized to no-substrate negative control
Lum_{cpd}—luminescence of test compound
Lum_{neg}—luminescence of no-substrate negative control
Lumₚₒₛ—luminescence of positive control

IC₅₀ parameter is determined by the GraphPad Prism 5.0 software

[log(inhibitor) vs. response−Variable slope (four parameters)].

IC₅₀ values of compounds of the present invention are shown in Table 2 below.

### BRK (PTK6 Protein Tyrosine Kinase 6) IC50 Determination Assay

In vitro kinase assay used to determine IC₅₀ values for tested inhibitors is based on a modified ADP-Glo™ Max Assay system (Promega) and consists of two parts:

1. Kinase reaction—performed in optimized conditions. At this step BRK phosphorylates its substrate Poly(Glu, Tyr) sodium salt [Glu:Tyr (4:1)] using ATP as a source of phosphate to produce phosphorylated substrate and ADP. Reaction is performed at Km values for ATP and substrate using optimized buffer composition and time of the reaction. BRK (PTK6 protein tyrosine kinase 6) used in the experiments is commercially available (Carna Bioscience, cat. No. 08-165).

2. Detection of ADP as a product of the reaction using ADP-Glo™ system. This part is conducted by using the commercially available kit ADP-Glo™ Max Assay (Promega, cat. No. V7001) according to manufacturer's instruction. Reproducibility and reliability of this modification is confirmed in an optimization process.

Test compounds are dissolved in DMSO and then transferred to the V-bottom 96-well plate. For IC50 determination nine 10× serial dilutions starting from 66.7 μM are prepared.

Two mixes are prepared on ice: Mix 1—containing appropriate kinase amount in 2× reaction buffer and Mix 2—containing concentrated substrate (Poly(Glu, Tyr) sodium salt) and ATP in MilliQ water. 15 μL per well of Mix 1 is transferred to assay wells of 96-well white plate. Next, 2 μl of 15× concentrated test compound in DMSO is added to Mix 1 for 20 min pre-incubation, followed by addition of Mix 2 (13 μl/well). Total reaction volume is 30 μL per well. Samples are tested in duplicates. Final concentration of DMSO in the reaction is 6.7%. Conditions needed for performing BRK in vitro kinase assay are given below:

| | Final | |
|---|---|---|
| | concentration/ | |
| Reagent/condition | final condition | |
| Buffer | 50 mM Tris, | |
| | pH 7.5 | |
| MgCl₂ | 40 | mM |
| NaCl | 25 | mM |
| DTT | 1 | mM |
| ATP (Km) (ultrapure, from | 1600 | uM |
| ADP-Glo ™ kit) | | |
| Poly(Glu, Tyr) sodium salt [Glu:Tyr | 160 | uM |
| (4:1)] (Sigma, cat. No.P0275-500MG) | | |
| PTK6 protein tyrosine kinase 6) | 75 | nM |
| (Carna Bioscience, cat. No. 08-165) | | |
| Time of reaction | 45 | min |

| | |
|---|---|
| Temperature of reaction | RT |

This protocol is based on Technical Bulletin, ADP-Glo™ Kinase Assay (Promega) and is adapted to 96-well plate containing 30 μL reaction mixture. 30 μL of ADP-Glo™ Reagent is added to each well of 96-well plate containing 30 μL of reaction mixture. The plate is incubated for 90 minutes on a shaker at RT. 60 μL of ADP-Glo™ Max Detection Solution is added to each well of 96-well plate containing 60 μL of the solution (ratio of kinase reaction volume to ADP-Glo™ Reagent volume to Kinase Detection Solution volume is maintained at 1:1:2). Plate is incubated for 40 minutes on a shaker at RT, protected from light. Luminescence is measured in the plate reader Synergy 2 (BioTek). Luminescent readouts for compounds tested in 8 concentrations (routinely from 66.7 μM in 5-fold serial dilution step) in duplicates, as well as for high control (complete reaction mix w/DMSO vehicle control), are first normalized to no-substrate low control by its subtraction. In the next step, % of normalized positive control is calculated for each data point and plotted against test compound concentration:

$$\% of control = 100 \% \times \frac{( {Lum}_{cpd} - {Lum}_{neg} )}{( {Lum}_{pos} - {Lum}_{neg} )}$$

% of control—percent of positive control normalized to no-substrate negative control
Lumcpd—luminescence of test compound
Lumneg—luminescence of no-substrate negative control
Lumpos—luminescence of positive control

IC₅₀ parameter is determined by the GraphPad Prism 6.0 software using 4-parameter model.

Compounds are classified according to their IC₅₀ values in the assays described above into three groups:

Group A IC₅₀ is in the range of ≥1 nM to <1 μM

Group B IC₅₀ is in the range of ≥1 μM to <10 μM

Group C IC₅₀ is in the range of ≥10 μM to ≤100 μM

TABLE 2

| Compound No. | Example No. | PFKFB3 IC50 | BRK IC50 |
|---|---|---|---|
| 1 | 52 | A | B |
| 2 | 1 | A | A |
| 3 | 2 | B | A |
| 4 | 3 | A | A |
| 5 | 4 | B | A |
| 8 | 88 | B | C |
| 10 | 6 | A | A |
| 11 | 7 | B | B |
| 12 | 83 | B | B |
| 14 | 8 | A | B |
| 15 | 9 | B | B |
| 16 | 11 | A | A |
| 19 | 12 | A | A |
| 21 | 14 | B | B |
| 22 | 15 | A | A |
| 23 | 16 | A | A |
| 25 | Intermediate 32 | B | C |
| 26 | Intermediate 37 | C | B |
| 27 | 17 | B | A |
| 28 | 18 | B | A |
| 30 | 19 | B | A |
| 31 | 20 | A | A |
| 32 | 21 | B | A |
| 33 | 115 | B | A |
| 34 | 114 | A | C |
| 35 | 22 | A | B |
| 36 | 23 | A | A |
| 37 | 24 | A | A |
| 39 | 25 | B | A |
| 42 | 113 | A | B |
| 44 | 92 | B | A |
| 45 | 117 | A | C |
| 46 | 116 | C | B |
| 47 | 59 | A | B |
| 49 | 94 | A | B |
| 52 | 26 | B | A |
| 53 | 27 | B | A |
| 54 | 28 | B | A |
| 55 | 29 | B | A |
| 56 | 30 | A | A |
| 57 | 31 | B | A |
| 59 | 32 | A | A |
| 60 | 61 | B | A |
| 61 | 97 | B | A |
| 62 | Intermediate 23 | C | B |
| 64 | 102 | C | B |
| 65 | 98 | C | B |
| 66 | 33 | A | A |
| 67 | 60 | C | B |
| 69 | 69 | A | A |
| 70 | Intermediate 7 | B | A |
| 71 | Intermediate 6 | B | A |
| 72 | 70 | A | A |
| 73 | 76 | B | A |
| 74 | Intermediate 27 | C | B |
| 75 | 80 | C | B |
| 76 | 39 | B | B |
| 77 | Intermediate 28 | C | A |
| 78 | 81 | B | B |
| 79 | 40 | A | A |
| 80 | 41 | B | A |
| 81 | 10 | A | C |
| 82 | 100 | C | B |
| 84 | 35 | B | B |
| 85 | 36 | B | B |
| 86 | 62 | B | A |
| 87 | 63 | B | A |
| 88 | 37 | B | A |
| 89 | 38 | B | A |
| 90 | 34 | A | A |
| 91 | 53 | A | B |
| 92 | 54 | A | A |
| 93 | 104 | B | B |
| 94 | 45 | B | A |
| 95 | 71 | A | A |
| 96 | Intermediate 24 | B | A |
| 97 | 42 | B | B |
| 98 | 109 | A | B |
| 99 | 72 | B | A |
| 100 | 75 | B | A |
| 101 | 73 | B | A |
| 102 | 107 | A | A |
| 103 | 43 | A | A |
| 104 | 46 | A | A |
| 105 | 57 | A | A |
| 106 | 74 | A | A |
| 107 | 108 | A | B |
| 108 | 47 | A | A |
| 109 | 66 | B | A |
| 110 | 68 | A | A |
| 112 | 55 | A | A |
| 113 | 111 | C | C |
| 115 | 56 | A | B |
| 116 | 50 | B | A |
| 117 | 64 | B | A |
| 119 | 13 | A | A |
| 120 | 82 | C | B |
| 121 | 65 | B | A |
| 122 | 110 | A | B |
| 123 | 58 | A | A |
| 124 | 67 | B | A |
| 125 | 48 | A | A |
| 126 | 77 | A | B |
| 127 | 78 | A | A |
| 128 | 51 | B | A |
| 129 | 79 | A | A |
| 130 | 49 | B | A |
| 131 | 118 | B | A |
| 132 | 119 | A | A |
| 133 | 120 | B | A |
| 134 | 121 | A | A |
| 135 | Intermediate 73 | B | B |
| 136 | 122 | B | B |
| 137 | Intermediate 74 | B | A |
| 138 | 123 | B | B |
| 139 | 124 | A | B |
| 140 | 125 | A | B |
| 141 | 126 | B | B |
| 142 | 127 | B | B |
| 143 | 128 | C | |
| 144 | 129 | C | |
| 145 | 130 | C | |
| 146 | 131 | C | |
| 147 | 132 | B | |
| 148 | 133 | C | |
| 149 | 134 | C | |
| 150 | 135 | C | |
| 151 | 136 | C | |
| 152 | 137 | B | |
| 153 | 138 | B | |
| 154 | 139 | B | |
| 155 | 140 | B | |
| 156 | 141 | B | |
| 157 | 142 | A | |
| 158 | 143 | B | |
| 159 | 144 | C | |
| 160 | 145 | B | |
| 161 | 146 | B | |
| 162 | 147 | B | |
| 163 | 148 | A | |
| 164 | 149 | B | |
| 165 | 150 | B | |
| 166 | 151 | B | |
| 167 | 152 | B | |
| 168 | 153 | A | |
| 169 | 154 | A | |
| 170 | 155 | C | |
| 171 | 156 | B | |
| 172 | 157 | B | |
| 173 | 158 | B | |
| 177 | 162 | C | |
| 178 | 163 | C | |
| 179 | 164 | B | |
| 180 | 165 | B | |
| 181 | 166 | B | |
| 182 | 167 | B | |
| 183 | 168 | B | |
| 184 | 169 | C | |
| 185 | 170 | B | |
| 186 | 171 | C | |
| 187 | 172 | C | |
| 188 | 173 | C | |
| 189 | 174 | C | |
| 190 | 175 | C | |
| 191 | 176 | C | |
| 192 | 177 | C | |
| 193 | 178 | C | |
| 194 | 179 | B | |
| 195 | 180 | C | |
| 196 | 181 | C | |
| 197 | 182 | C | |
| 198 | 183 | C | |
| 199 | 184 | C | |
| 200 | 185 | C | |
| 201 | 186 | C | |
| 202 | 187 | C | |
| 203 | 188 | C | |
| 204 | 189 | C | |
| 205 | 190 | C | |
| 206 | 191 | A | |
| 207 | 192 | C | |
| 208 | 193 | C | |
| 209 | 194 | C | |
| 210 | 195 | C | |
| 211 | 196 | C | |
| 212 | 197 | C | |
| 213 | 198 | C | |
| 214 | 199 | C | |
| 215 | 200 | C | |
| 216 | 201 | C | |
| 217 | 202 | C | |
| 219 | 203 | A | |
| 220 | 204 | C | |
| 221 | 205 | A | |
| 222 | 206 | B | |
| 223 | 207 | B | |
| 224 | 208 | A | |
| 225 | 209 | A | |
| 226 | 210 | A | |
| 227 | 211 | B | |
| 228 | 212 | C | |
| 229 | 213 | C | |
| 230 | 214 | B | |
| 231 | 215 | A | |
| 232 | 216 | A | |
| 233 | 217 | A | |
| 234 | 218 | B | |
| 235 | 219 | B | |
| 236 | 220 | B | |
| 237 | 221 | A | |
| 238 | 222 | A | |
| 239 | 223 | A | |
| 240 | 224 | A | |
| 241 | 225 | A | |
| 242 | 226 | A | |
| 243 | 227 | A | A |
| 244 | 228 | A | A |
| 245 | 229 | A | A |
| 246 | 230 | A | A |
| 247 | 231 | A | A |
| 248 | 232 | A | A |
| 249 | 233 | A | B |
| 250 | 234 | A | A |
| 251 | 235 | B | C |
| 252 | 236 | B | |
| 253 | 237 | A | |
| 254 | 238 | B | |
| 255 | 239 | A | |
| 257 | 241 | B | |
| 258 | 242 | A | |
| 259 | 243 | A | |
| 260 | 244 | A | |
| 261 | 245 | B | |
| 262 | 246 | B | |
| 263 | 247 | A | |
| 264 | 248 | B | |
| 265 | 249 | A | |
| 266 | 250 | A | |
| 267 | 251 | A | |
| 268 | 252 | A | |
| 269 | 253 | A | |
| 270 | 254 | A | |
| 271 | 255 | A | |
| 272 | 256 | B | |
| 273 | 257 | B | |
| 274 | 258 | B | |
| 275 | 259 | A | |
| 276 | 260 | A | |
| 277 | 261 | B | |
| 278 | 262 | B | |
| 279 | 263 | B | |
| 280 | 264 | B | |
| 281 | 265 | A | |
| 282 | 266 | B | |
| 283 | 267 | B | |
| 284 | 268 | C | |
| 285 | 269 | B | |
| 286 | 270 | 6 | |
| 287 | 271 | A | |
| 288 | 272 | A | |
| 289 | 273 | B | |
| 290 | 274 | B | |
| 291 | 275 | C | |
| 292 | 276 | C | |
| 293 | 277 | B | |
| 294 | 278 | B | |
| 295 | 279 | A | |
| 296 | 280 | A | |
| 297 | 281 | C | |
| 298 | 282 | A | |
| 299 | 283 | A | |
| 300 | 284 | A | |
| 301 | 285 | B | |
| 302 | 286 | A | |
| 304 | 288 | A | |
| 305 | 289 | B | |
| 306 | 290 | B | |
| 307 | 291 | C | |
| 308 | 292 | A | |
| 309 | 293 | B | |
| 310 | 294 | B | |
| 311 | 295 | A | |
| 312 | 296 | B | |
| 313 | 297 | A | |
| 314 | 298 | A | |
| 315 | 299 | A | |
| 316 | 300 | A | |
| 317 | 301 | B | |
| 318 | 302 | A | |
| 319 | 303 | A | |
| 320 | 304 | A | |
| 321 | 305 | B | |
| 322 | 306 | A | |
| 323 | 307 | A | |
| 324 | 308 | A | |
| 325 | 309 | A | |
| 326 | 310 | A | |
| 327 | 311 | B | |
| 328 | 312 | A | |

1. Compound of formula (I) 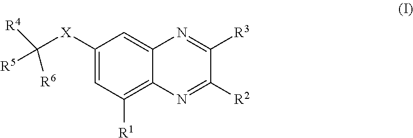
wherein
X denotes N—R⁷ or O;
R¹ denotes Ar^{X}, Ar^{X}—Ar^{Y}, Ar^{X}-Hetar^{Y}, Ar^{X}-Hetcyc^{Y}, Ar^{X}-LA^{Z}-Ar^{Y}, Ar^{X}-LA^{Z}-Hetar^{Y}, Ar^{X}-LA^{Z}-Hetcyc^{Y}, Hetar^{X}, Hetar^{X}-Ar^{Y}, Hetar^{X}-Hetar^{Y}, Hetar^{X}-Hetcyc^{Y}, Hetar^{X}-LA^{Z}-Ar^{Y}, Hetar^{X}-LA^{Z}-Hetar^{Y}, Hetar^{X}-LA^{Z}-Hetcyc^{Y}, Hetcyc^{X}, Hetcyc^{X}-Ar^{Y}, Hetcyc^{X}-Hetar^{Y}, Hetcyc^{X}-Hetcyc^{Y}, Hetcyc^{X}-LA^{Z}-Ar^{Y}, Hetcyc^{X}-LA^{Z}-Hetar^{Y}, Hetcyc^{X}-LA^{Z}-Hetcyc^{Y}, CA^{X};
R² and R³ denote independently from each other H, OH, SH, unsubstituted straight-chain or branched —C₁₋₆-alkyl, straight-chain or branched —C₂₋₆-alkenyl, unsubstituted straight-chain or branched —O—C₁₋₆-alkyl, straight-chain or branched —S—C₁₋₆-alkyl, Hal, —CN, —C(═O)—NH₂, —C(═O)—NH(C₁₋₄-alkyl), —C(═O)—N(C₁₋₄-alkyl)₂, —NH₂, —NH(C₁₋₄-alkyl), —N(C₁₋₄-alkyl)₂ which C₁₋₄-alkyl substituents may be the same or different and may be straight-chain or branched;
R⁴ denotes Ar^{X}, Ar^{X}—Ar^{Y}, Ar^{X}-Hetar^{Y}, Ar^{X}-Hetcyc^{Y}, Ar^{X}-LA^{Z}-Ar^{Y}, Ar^{X}-LA^{Z}-Hetar^{Y}, Ar^{X}-LA^{Z}-Hetcyc^{Y}, Hetar^{X}, Hetar^{X}-Ar^{Y}, Hetar^{X}-Hetar^{Y}, Hetar^{X}-Hetcyc^{Y}, Hetar^{X}-LA^{Z}-Ar^{Y}, Hetar^{X}-LA^{Z}-Hetar^{Y}, Hetar^{X}-LA^{Z}-Hetcyc^{Y}, Hetcyc^{X}, Hetcyc^{X}-Ar^{Y}, Hetcyc^{X}-Hetar^{Y}, Hetcyc^{X}-Hetcyc^{Y}, Hetcyc^{X}-LA^{Z}-Ar^{Y}, Hetcyc^{X}-LA^{Z}-Hetar^{Y}, Hetcyc^{X}-LA^{Z}-Hetcyc^{Y}, LA^{X}, LA^{Z}-Ar^{Y}, LA^{Z}-Hetar^{Y}, LA^{Z}-Hetcyc^{Y}, CA^{X};
R⁵ denotes H, Ar^{X}, Ar^{X}—Ar^{Y}, Ar^{X}-Hetar^{Y}, Ar^{X}-Hetcyc^{Y}, Ar^{X}-LA^{Z}-Ar^{Y}, Ar^{X}-LA^{Z}-Hetar^{Y}, Ar^{X}-LA^{Z}-Hetcyc^{Y}, Hetar^{X}, Hetar^{X}-Ar^{Y}, Hetar^{X}-Hetar^{Y}, Hetar^{X}-Hetcyc^{Y}, Hetar^{X}-LA^{Z}-Ar^{Y}, Hetar^{X}-LA^{Z}-Hetar^{Y}, Hetar^{X}-LA^{Z}-Hetcyc^{Y}, Hetcyc^{X}, Hetcyc^{X}-Ar^{Y}, Hetcyc^{X}-Hetar^{Y}, Hetcyc^{X}-Hetcyc^{Y}, Hetcyc^{X}-LA^{Z}-Ar^{Y}, Hetcyc^{X}-LA^{Z}-Hetar^{Y}, Hetcyc^{X}-LA^{Z}-Hetcyc^{Y}, LA^{X}, LA^{Z}-Ar^{Y}, LA^{Z}-Hetar^{Y}, LA^{Z}-Hetcyc^{Y}, CA^{X}, —CN, —NO₂, —SO₂NH₂, —SO₂NHR^{X7}, —SO₂NR^{X7}R^{X8}, —NH—SO₂—R^{X9}, —NR^{X7}—SO₂—R^{X9}, —SO₂—R^{X9}, —NH₂, —NHR^{X7}, —NR^{X7}R^{X8}, —OH, —O—R^{X9}, —CHO, —C(═O)—R^{X9}, —NH—(C₁₋₃-alkylene)-C(═O)—NH₂, —NH—(C₁₋₃-alkylene)-C(═O)—NHR^{X7}, —NH—(C₁₋₃-alkylene)-C(═O)—NR^{X7}R^{X8}, —NH—C(═O)—R^{X9}, —NR^{X7}—C(═O)—R^{X9}, —C(═O)—NH₂, —C(═O)—NHR^{X7}, —C(═O)—NR^{X7}R^{X8}, —C(═O)OH, —C(═O)OR^{X9};
or
R⁴ and R⁵ form together with the carbon atom to which they are attached to a saturated or partially unsaturated ring system A which ring system A is mono- or bicyclic and has 3, 4, 5, 6, 7, 8, 9, 10, 11 ring atoms and may contain no hetero ring atom or 1, 2, 3 hetero ring atom(s) independently from each other selected from N, O and/or S that ring system A may be unsubstituted or mono-, di- or trisubstituted with independently from each other R^{A1}, R^{A2}, R^{A3};
R⁶ denotes denotes H, Ar^{X}, Ar^{X}—Ar^{Y}, Ar^{X}-Hetar^{Y}, Ar^{X}-Hetcyc^{Y}, Ar^{X}-LA^{Z}-Ar^{Y}, Ar^{X}-LA^{Z}-Hetar^{Y}, Ar^{X}-LA^{Z}-Hetcyc^{Y}, Hetar^{X}, Hetar^{X}-Ar^{Y}, Hetar^{X}-Hetar^{Y}, Hetar^{X}-Hetcyc^{Y}, Hetar^{X}-LA^{Z}-Ar^{Y}, Hetar^{X}-LA^{Z}-Hetar^{Y}, Hetar^{X}-LA^{Z}-Hetcyc^{Y}, Hetcyc^{X}, Hetcyc^{X}-Ar^{Y}, Hetcyc^{X}-Hetar^{Y}, Hetcyc^{X}-Hetcyc^{Y}, Hetcyc^{X}-LA^{Z}-Ar^{Y}, Hetcyc^{X}-LA^{Z}-Hetar^{Y}, Hetcyc^{X}-LA^{Z}-Hetcyc^{Y}, LA^{X}, LA^{Z}-Ar^{Y}, LA^{Z}-Hetar^{Y}, LA^{Z}-Hetcyc^{Y}, CA^{X};
or
R⁵ and R⁶ form together with the carbon atom to which they are attached to a saturated or partially unsaturated ring system D which ring system D is mono- or bicyclic and has 3, 4, 5, 6, 7, 8, 9, 10, 11 ring atoms and may contain no hetero ring atom or 1, 2, 3 hetero ring atom(s) independently from each other selected from N, O and/or S that ring system D may be unsubstituted or mono-, di- or trisubstituted with independently from each other R^{D1}, R^{D2}, R^{D3};
or
R⁵ and R⁶ form together with the carbon atom to which they are attached to a C═CHR^{D4} moiety;
R⁷ denotes H, Hetar^{X}, Hetcyc^{X}, LA^{X}, CA^{X};
Ar^{X} denotes a mono-, bi- or tricyclic aromatic ring system with 5, 6, 7, 8, 9, 10, 11, 12, 13, 14 ring carbon atoms which ring system may be unsubstituted or mono-, di- or trisubstituted with independently from each other R^{X1}, R^{X2}, R^{X3};
Ar^{Y} denotes a mono-, bi- or tricyclic aromatic ring system with 5, 6, 7, 8, 9, 10, 11, 12, 13, 14 ring carbon atoms which ring system may be unsubstituted or mono-, di- or trisubstituted with independently from each other R^{Y1}, R^{Y2}, R^{Y3};
Hetar^{X} denotes a mono, bi- or tricyclic aromatic ring system with 5, 6, 7, 8, 9, 10, 11, 12, 13, 14 ring atoms wherein 1, 2, 3, 4, 5 of said ring atoms is/are a hetero atom(s) selected from N, O and/or S and the remaining are carbon atoms, wherein that aromatic ring system may be unsubstituted or mono-, di- or trisubstituted with independently from each other R^{X1}, R^{X2}, R^{X3};
Hetar^{Y} denotes a mono, bi- or tricyclic aromatic ring system with 5, 6, 7, 8, 9, 10, 11, 12, 13, 14 ring atoms wherein 1, 2, 3, 4, 5 of said ring atoms is/are a hetero atom(s) selected from N, O and/or S and the remaining are carbon atoms, wherein that aromatic ring system may be unsubstituted or mono-, di- or trisubstituted with independently from each other R^{Y1}, R^{Y2}, R^{Y3};
Hetcyc^{X} denotes a saturated or partially unsaturated mono-, bi- or tricyclic heterocycle with 3, 4, 5, 6, 7, 8, 9, 10, 11, 12, 13, 14 ring atoms wherein 1, 2, 3, 4, 5 ring atom(s) is/are heteroatom(s) selected from N, O and/or S and the remaining ring atoms are carbon atoms, wherein that heterocycle may be unsubstituted or mono-, di- or trisubstituted with R^{X4}, R^{X5}, R^{X6};
Hetcyc^{Y} denotes a saturated or partially unsaturated mono-, bi- or tricyclic heterocycle with 3, 4, 5, 6, 7, 8, 9, 10, 11, 12, 13, 14 ring atoms wherein 1, 2, 3, 4, 5 ring atom(s) is/are heteroatom(s) selected from N, O and/or S and the remaining ring atoms are carbon atoms, wherein that heterocycle may be unsubstituted or mono-, di- or trisubstituted with R^{Y4}, R^{Y5}, R^{Y6};
R^{X1}, R^{X2}, R^{X3} denote independently from each other other H, Hal, LA^{X}, CA^{X}, —CN, —NO₂, —SO₂NH₂, —SO₂NHR^{X7}, —SO₂NR^{X7}R^{X8}, —NH—SO₂—R^{X9}, —NR^{X7}—SO₂—R^{X9}, —S—R^{X9}, —S(═O)—R^{X9}, —SO₂—R^{X9}, —NH₂, —NHR^{X7}, —NR^{X7}R^{X8}, —OH, —O—R^{X9}, —CHO, —C(═O)—R^{X9}, —COOH, —C(═O)—O—R^{X9}, —C(═O)—NH₂, —C(═O)—NHR^{X7}, —C(═O)—NR^{X7}R^{X8}, —NH—(C₁₋₃-alkylene)-C(═O)—NH₂, —NH—(C₁₋₃-alkylene)-C(═O)—NHR^{X7}, —NH—(C₁₋₃-alkylene)-C(═O)—NR^{X7}R^{X8}, —NH—C(═O)—R^{X9}, —NR^{X7}—C(═O)—R^{X9},
or
two of R^{X1}, R^{X2}, R^{X3} form a divalent alkylene chain with 3, 4, 5 chain carbon atoms wherein 1 or 2 of non-adjacent CH₂ groups of the divalent alkylene chain may be replaced independently from each other by —N(H)—, —N(C₁₋₆-alkyl)-, —N(—C(═O)—C₁₋₄-alkyl)-, —O— —wherein that C₁₋₆-alkyl and C₁₋₄-alkyl radicals may be straight-chain or branched—and wherein 2 adjacent CH₂ groups may together be replaced by a —CH═CH— moiety, which divalent alkylene chain may be unsubstituted or mono- or di-substituted with independently from each other straight-chain or branched C₁₋₆-alkyl or ═O (oxo);
R^{X4}, R^{X5}, R^{X6} denote independently from each other H, Hal, LA^{X}, CA^{X}, —CN, —NO₂, —SO₂NH₂, —SO₂NHR^{X7}, —SO₂NR^{X7}R^{X8}, —NH—SO₂—R^{X9}, —NR^{X7}—SO₂—R^{X9}, —S—R^{X9}, —S(═O)—R^{X9}, —SO₂—R^{X9}, —NH₂, —NHR^{X7}, —NR^{X7}R^{X8}, —OH, —O—R^{X9}, —CHO, —C(═O)—R^{X9}, —COOH, —C(═O)—O—R^{X9}, —C(═O)—NH₂, —C(═O)—NHR^{X7}, —C(═O)—NR^{X7}R^{X8}, —NH—(C₁₋₃-alkylene)-C(═O)—NH₂, —NH—(C₁₋₃-alkylene)-C(═O)—NHR^{X7}, —NH—(C₁₋₃-alkylene)-C(═O)—NR^{X7}R^{X8}, —NH—C(═O)—R^{X9}, —NR^{X7}—C(═O)—R^{X9}, oxo (═O);
R^{Y1}, R^{Y2}, R^{Y3} denote independently from each other H, Hal, LA^{Y}, CA^{Y}, —CN, —NO₂, —SO₂NH₂, —SO₂NHR^{Y7}, —SO₂NR^{Y7}R^{Y8}, —NH—SO₂—R^{Y9}, —NR^{Y7}—SO₂—R^{Y9}, —S—R^{Y9}, —S(═O)—R^{Y9}, —SO₂—R^{Y9}, —NH₂, —NHR^{Y7}, —NR^{Y7}R^{Y8}, —OH, —O—R^{Y9}, —CHO, —C(═O)—R^{Y9}, —COOH, —C(═O)—O—R^{Y9}, —C(═O)—NH₂, —C(═O)—NHR^{Y7}, —C(═O)—NR^{Y7}R^{Y8}, —NH—(C₁₋₃-alkylene)-C(═O)—NH₂, —NH—(C₁₋₃-alkylene)-C(═O)—NHR^{Y7}, —NH—(C₁₋₃-alkylene)-C(═O)—NR^{Y7}R^{Y8}, —NH—C(═O)—R^{Y9}, —NR^{Y7}—C(═O)—R^{Y9}
or
two of R^{Y1}, R^{Y2}, R^{Y3} form a divalent alkylene chain with 3, 4, 5 chain carbon atoms wherein 1 or 2 non-adjacent CH₂ groups of the divalent alkylene chain may be replaced independently from each other by —N(H)—, —N(C₁₋₆-alkyl)-, —N(—C(═O)—C₁₋₄-alkyl), —O— —wherein that C₁₋₆-alkyl and C₁₋₄-alkyl radicals may be straight-chain or branched—and wherein 2 adjacent CH₂ groups may together be replaced by a —CH═CH— moiety, which divalent alkylene chain may be unsubstituted or mono- or di-substituted with independently from each other straight-chain or branched —C₁₋₆-alkyl or ═O (oxo);
R^{Y4}, R^{Y5}, R^{Y6} denote independently from each other H, Hal, LA^{Y}, CA^{Y}, —CN, —NO₂, —SO₂NH₂, —SO₂NHR^{Y7}, —SO₂NR^{Y7}R^{Y8}, —NH—SO₂—R^{Y9}, —NR^{Y7}—SO₂—R^{Y9}, —S—R^{Y9}, —S(═O)—R^{Y9}, —SO₂—R^{Y9}, —NH₂, —NHR^{Y7}, —NR^{Y7}R^{Y8}, —OH, —O—R^{Y9}, —CHO, —C(═O)—R^{Y9}, —COOH, —C(═O)—O—R^{Y9}, —C(═O)—NH₂, —C(═O)—NHR^{Y7}, —C(═O)—NR^{Y7}R^{Y8}, —NH—(C₁₋₃-alkylene)-C(═O)—NH₂, —NH—(C₁₋₃-alkylene)-C(═O)—NHR^{Y7}, —NH—(C₁₋₃-alkylene)-C(═O)—NR^{Y7}R^{Y8}, —NH—C(═O)—R^{Y9}, —NR^{Y7}—C(═O)—R^{Y9}, Oxo (═O);
LA^{X} denotes straight-chain or branched C₁₋₆-alkyl which may be unsubstituted or mono-, di- or trisubstituted with independently from each other Hal, —CN, —NO₂, —SO₂NH₂, —SO₂NHR^{X7}, —SO₂NR^{X7}R^{X8}, —NH—SO₂—R^{X9}, —NR^{X7}—SO₂—R^{X9}, —S—R^{X9}, —S(═O)—R^{X9}, —SO₂—R^{X9}, —NH₂, —NHR^{X7}, —NR^{X7}R^{X8}, —OH, —O—R^{X9}, —CHO, —C(═O)—R^{X9}, —COOH, —C(═O)—O—R^{X9}, —C(═O)—NH₂, —C(═O)—NHR^{X7}, —C(═O)—NR^{X7}R^{X8}, —NH—(C₁₋₃-alkylene)-C(═O)—NH₂, —NH—(C₁₋₃-alkylene)-C(═O)—NHR^{X7}, —NH—(C₁₋₃-alkylene)-C(═O)—NR^{X7}R^{X8}, —NH—C(═O)—R^{X9}, —NR^{X7}—C(═O)—R^{X9}, oxo (═O), wherein 1 or 2 non-adjacent CH₂ groups of the C₁₋₆-alkyl radical may independently from each other be replaced by O, S, N(H) or N—R^{X7} and/or 1 or 2 non-adjacent CH groups of the C₁₋₆-alkyl radical may independently from each other be replaced by N;
LA^{Y} denotes straight-chain or branched C₁₋₆-alkyl which may be unsubstituted or mono-, di- or trisubstituted with independently from each other Hal, —CN, —NO₂, —SO₂NH₂, —SO₂NHR^{Y7}, —SO₂NR^{Y7}R^{Y8}, —NH—SO₂—R^{Y9}, —NR^{Y7}—SO₂—R^{Y9}, —S—R^{Y9}, S(═O)—R^{Y9}, —SO₂—R^{Y9}, —NH₂, —NHR^{Y7}, —NR^{Y7}R^{Y8}, —OH, —O—R^{Y9}, —CHO, —C(═O)—R^{Y9}, —COOH, —C(═O)—O—R^{Y9}, —C(═O)—NH₂, —C(═O)—NHR^{Y7}, —C(═O)—NR^{Y7}R^{Y8}, —NH—(C₁₋₃-alkylene)-C(═O)—NH₂, —NH—(C₁₋₃-alkylene)-C(═O)—NHR^{Y7}, —NH—(C₁₋₃-alkylene)-C(═O)—NR^{Y7}R^{Y8}, —NH—C(═O)—R^{Y9}, —NR^{Y7}—C(═O)—R^{Y9}, oxo (═O), wherein 1 or 2 non-adjacent CH₂ groups of the C₁₋₆-alkyl radical may independently from each other be replaced by O, S, N(H) or N—R^{Y7} and/or 1 or 2 non-adjacent CH groups of the C₁₋₆-alkyl radical may independently from each other be replaced by N;
LA^{Z} denotes a divalent straight-chain or branched C₁₋₆-alkylene radical which alkylene radical may be unsubstituted or mono-, di- or trisubstituted with independently from each other Hal, —CN, —NO₂, —SO₂NH₂, —SO₂NHR^{Z7}, —SO₂NR^{Z7}R^{Z8}, —NH—SO₂—R^{Z9}, —NR^{Z7}—SO₂—R^{Z9}, —S—R^{Z9}, —S(═O)—R^{Z9}, —SO₂—R^{Z9}, —NH₂, —NHR^{Z7}, —NR^{Z7}R^{Z8}, —OH, —O—R^{Z9}, —CHO, —C(═O)—R^{Z9}, —COOH, —C(═O)—O—R^{Z9}, —C(═O)—NH₂, —C(═O)—NHR^{Z7}, —C(═O)—NR^{Z7}R^{Z8}, —NH—(C₁₋₃-alkylene)-C(═O)—NH₂, —NH—(C₁₋₃-alkylene)-C(═O)—NHR^{Z7}, —NH—(C₁₋₃-alkylene)-C(═O)—NR^{Z7}R^{Z8}, —NH—C(═O)—R^{Z9}, —NR^{Z7}—C(═O)—R^{Z9}, oxo (═O), wherein 1 or 2 non-adjacent CH₂ groups of that divalent alkylene radical may be replaced independently from each other by O, S or —N(H) and/or 1 or 2 non-adjacent CH groups of that divalent alkylene radical may be replaced by N;
R^{X7}, R^{X8}, R^{Y7}, R^{Y8}, R^{Z7}, R^{Z8} denote independently from each other straight-chain or branched —C₁₋₆-alkyl, phenyl, a mono- or bicyclic aromatic ring system with 5, 6, 7, 8, 9, 10, 11 ring atoms wherein 1, 2, 3, 4 of said ring atoms is/are a hetero atom(s) selected from N, O and/or S and the remaining are carbon atoms and wherein that aromatic ring system may be unsubstituted or mono- or disubstituted with independently from each other straight-chain or branched C₁₋₆-alkyl or —O—C₁₋₆-alkyl or —NH₂, or a saturated monocyclic carbocycle with 3, 4, 5, 6, 7 carbon atoms
or
each pair R^{X7} and R^{X8}; R^{Y7} and R^{Y8}; R^{Z7} and R^{Z8} form independently from each other pair together with the nitrogen atom to which they are attached to a 3, 4, 5, 6 or 7 membered heterocycle wherein that heterocycle may not contain any further heteroatom or may contain besides said nitrogen atom one further hetero ring atom selected from N, O and S, wherein, if that further hetero atom is N, that further N may be substituted with H or straight-chain or branched —C₁₋₆-alkyl;
R^{X9}, R^{Y9}, R^{Z9} denote independently from each other straight-chain or branched —C₁₋₆-alkyl, which may be unsubstituted or mono-, di- or trisubstituted with Hal, phenyl, a mono- or bicyclic aromatic ring system with 5, 6, 7, 8, 9, 10, 11 ring atoms wherein 1, 2, 3, 4 of said ring atoms is/are a hetero atom(s) selected from N, O and/or S and the remaining are carbon atoms and wherein that aromatic ring system may be unsubstituted or mono- or disubstituted with independently from each other straight-chain or branched C₁₋₆-alkyl or —O—C₁₋₆-alkyl or —NH₂, or a saturated monocyclic carbocycle with 3, 4, 5, 6, 7 carbon atoms;
R^{A1}, R^{A2}, R^{A3} denote independently from each other H, Hal, Ar^{X}, Hetar^{X}, Hetcyc^{X}, LA^{X}, CA^{X}, —CN, —NO₂, —SO₂NH₂, —SO₂NHR^{X7}, —SO₂NR^{X7}R^{X8}, —NH—SO₂—R^{X9}, —NR^{X7}—SO₂—R^{X9}, —S—R^{X9}, —S(═O)—R^{X9}, —SO₂—R^{X9}, —NH₂, —NHR^{X7}, —NR^{X7}R^{X8}, —OH, —O—R^{X9}, —CHO, —C(═O)—R^{X9}, —COOH, —C(═O)—O—R^{X9}, —C(═O)—NH₂, —C(═O)—NHR^{X7}, —C(═O)—NR^{X7}R^{X8}, —NH—(C₁₋₃-alkylene)-C(═O)—NH₂, —NH—(C₁₋₃-alkylene)-C(═O)—NHR^{X7}, —NH—(C₁₋₃-alkylene)-C(═O)—NR^{X7}R^{X8}, —NH—C(═O)—R^{X9}, —NR^{X7}—C(═O)—R^{X9}, oxo (═O);
or
two of R^{A1}, R^{A2} and R^{A3} form together with one carbon atom of that ring system A to which they both are attached to a saturated or partially unsaturated ring system E which ring system E is mono- or bicyclic and has 3, 4, 5, 6, 7, 8, 9, 10 ring atoms and may contain no hetero atom or 1, 2, 3 hetero atom(s) independently from each other selected from N, O and/or S that ring system E may be unsubstituted or mono-, di- or trisubstituted with independently from each other R^{E1}, R^{E2}, R^{E3};
R^{D1}, R^{D2}, R^{D3}, R^{E1}, R^{E2}, R^{E3} denote independently from each other H, Hal, Ar^{X}, Hetar^{X}, Hetcyc^{X}, LA^{X}, CA^{X}, —CN, —NO₂, —SO₂NH₂, —SO₂NHR^{X7}, —SO₂NR^{X7}R^{X8}, —NH—SO₂—R^{X9}, —NR^{X7}—SO₂—R^{X9}, —S—R^{X9}, S(═O)—R^{X9}, —SO₂—R^{X9}, —NH₂, —NHR^{X7}, —NR^{X7}R^{X8}, —OH, —O—R^{X9}, —CHO, —C(═O)—R^{X9}, —COOH, —C(═O)—O—R^{X9}, —C(═O)—NH₂, —C(═O)—NHR^{X7}, —C(═O)—NR^{X7}R^{X8}, —NH—(C₁₋₃-alkylene)-C(═O)—NH₂, —NH—(C₁₋₃-alkylene)-C(═O)—NHR^{X7}, —NH—(C₁₋₃-alkylene)-C(═O)—NR^{X7}R^{X8}, —NH—C(═O)—R^{X9}, —NR^{X7}—C(═O)—R^{X9}, oxo (═O);
R^{D4} denotes H, Hal, Ar^{X}, Hetar^{X}, Hetcyc^{X}, LA^{X}, CA^{X}, —CN, —NO₂, —SO₂NH₂, —SO₂NHR^{X7}, —SO₂NR^{X7}R^{X8}, —NH—SO₂—R^{X9}, —NR^{X7}—SO₂—R^{X9}, —S—R^{X9}, S(═O)—R^{X9}, —SO₂—R^{X9}, —NH₂, —NHR^{X7}, —NR^{X7}R^{X8}, —OH, —O—R^{X9}, —CHO, —C(═O)—R^{X9}, —COOH, —C(═O)—O—R^{X9}, —C(═O)—NH₂, —C(═O)—NHR^{X7}, —C(═O)—NR^{X7}R^{X8}, —NH—(C₁₋₃-alkylene)-C(═O)—NH₂, —NH—(C₁₋₃-alkylene)-C(═O)—NHR^{X7}, —NH—(C₁₋₃-alkylene)-C(═O)—NR^{X7}R^{X8}, —NH—C(═O)—R^{X9}, —NR^{X7}—C(═O)—R^{X9};
CA^{X}, CA^{Y} denote independently from each other a saturated monocyclic carbocycle with 3, 4, 5, 6, 7 carbon atoms which carbocycle may be unsubstituted or mono- or disubstituted with independently from each other R^{CA1}, R^{CA2};
R^{CA1}, R^{CA2} denote independently from each other H, Hal, Ar^{X}, Ar^{X}—Ar^{Y}, Ar^{X}-Hetar^{Y}, Ar^{X}-Hetcyc^{Y}, Ar^{X}-LA^{Z}-Ar^{Y}, Ar^{X}-LA^{Z}-Hetar^{Y}, Ar^{X}-LA^{Z}-Hetcyc^{Y}, Hetar^{X}, Hetar^{X}-Ar^{Y}, Hetar^{X}-Hetar^{Y}, Hetar^{X}-Hetcyc^{Y}, Hetar^{X}-LA^{Z}-Ar^{Y}, Hetar^{X}-LA^{Z}-Hetar^{Y}, Hetar^{X}-LA^{Z}-Hetcyc^{Y}, Hetcyc^{X}, Hetcyc^{X}-Ar^{Y}, Hetcyc^{X}-Hetar^{Y}, Hetcyc^{X}-Hetcyc^{Y}, Hetcyc^{X}-LA^{Z}-Ar^{Y}, Hetcyc^{X}-LA^{Z}-Hetar^{Y}, Hetcyc^{X}-LA^{Z}-Hetcyc^{Y}, LA^{X}, LA^{Z}-Ar^{Y}, LA^{Z}-Hetar^{Y}, LA^{Z}-Hetcyc^{Y}, —CN, —NO₂, —SO₂NH₂, —SO₂NHR^{X7}, —SO₂NR^{X7}R^{X8}, —NH—SO₂—R^{X9}, —NR^{X7}—SO₂—R^{X9}, —S—R^{X9}, S(═O)—R^{X9}, —SO₂—R^{X9}, —NH₂, —NHR^{X7}, —NR^{X7}R^{X8}, —OH, —O—R^{X9}, —CHO, —C(═O)—R^{X9}, —COOH, —C(═O)—O—R^{X9}, —C(═O)—NH₂, —C(═O)—NHR^{X7}, —C(═O)—NR^{X7}R^{X8}, —NH—(C₁₋₃-alkylene)-C(═O)—NH₂, —NH—(C₁₋₃-alkylene)-C(═O)—NHR^{X7}, —NH—(C₁₋₃-alkylene)-C(═O)—NR^{X7}R^{X8}, —NH—C(═O)—R^{X9}, —NR^{X7}—C(═O)—R^{X9}, Oxo (═O),
with the proviso that if R^{CA1} or R^{CA2} denotes Ar^{X}, Ar^{X}—Ar^{Y}, Ar^{X}-Hetar^{Y}, Ar^{X}-Hetcyc^{Y}, Ar^{X}-LA^{Z}-Ar^{Y}, Ar^{X}-LA^{Z}-Hetar^{Y}, Ar^{X}-LA^{Z}-Hetcyc^{Y}, Hetar^{X}, Hetar^{X}-Ar^{Y}, Hetar^{X}-Hetar^{Y}, Hetar^{X}-Hetcyc^{Y}, Hetar^{X}-LA^{Z}-Ar^{Y}, Hetar^{X}-LA^{Z}-Hetar^{Y}, Hetar^{X}-LA^{Z}-Hetcyc^{Y}, Hetcyc^{X}, Hetcyc^{X}-Ar^{Y}, Hetcyc^{X}-Hetar^{Y}, Hetcyc^{X}-Hetcyc^{Y}, Hetcyc^{X}-LA^{Z}-Ar^{Y}, Hetcyc^{X}-LA^{Z}-Hetar^{Y}, Hetcyc^{X}-LA^{Z}-Hetcyc^{Y}, LA^{Z}-Ar^{Y}, LA^{Z}-Hetar^{Y}, LA^{Z}-Hetcyc^{Y}, then Ar^{X}, Ar^{Y}, Hetar^{X}, Hetar^{Y}, Hetcyc^{X}, Hetcyc^{Y} may not be substituted with CA^{X} or CA^{Y};
Hal denotes F, Cl, Br, I;
or derivatives, N-oxides, prodrugs, solvates, tautomers or stereoisomers thereof as well as the physiologically acceptable salts of each of the foregoing, including mixtures thereof in all ratios.

2. Compound according to claim 1, or derivatives, prodrugs, solvates, tautomers or stereoisomers thereof as well as the physiologically acceptable salts of each of the foregoing, including mixtures thereof in all ratios,
wherein
X denotes N—R⁷ or O;
R¹ denotes Ar^{X}, Ar^{X}-Hetar^{Y}, Ar^{X}-Hetcyc^{Y}, Hetar^{X}, Hetcyc^{X}, Hetar^{X}-LA^{Z}-Ar^{Y};
R² and R³ denote independently from each other H, —OH, unsubstituted straight-chain or branched —C₁₋₆-alkyl, unsubstituted straight-chain or branched —O—C₁₋₆-alkyl, Hal, —CN, —C(═O)—NH₂;
R⁴ denotes Ar^{X}, Ar^{X}—Ar^{Y}, Ar^{X}-Hetar^{Y}, Ar^{X}-Hetcyc^{Y}, Hetar^{X}, Hetar^{X}-Ar^{Y}, Hetar^{X}-Hetar^{Y}, Hetar^{X}-Hetcyc^{Y}, Hetcyc^{X}, Hetcyc^{X}-Hetar^{Y}, Hetcyc^{X}-LA^{Z}-Ar^{Y}, LA^{X}, LA^{Z}-Hetar^{Y}, LA^{Z}-Hetcyc^{Y};
R⁵ denotes H, Hetar^{X}, Hetcyc^{X}, LA^{X}, CA^{X}, —C(═O)—NR^{X7}R^{X8};
or
R⁴ and R⁵ form together with the carbon atom to which they are attached to a saturated or partially unsaturated ring system A which ring system A is mono- or bicyclic and has 3, 4, 5, 6, 7, 8, 9, 10 ring atoms and may contain no hetero ring atom or 1, 2, 3 hetero ring atom(s) independently from each other selected from N, O and/or S that ring system A may be unsubstituted or mono-, di- or trisubstituted with independently from each other R^{A1}, R^{A2}, R^{A3};
R⁶ denotes denotes H, Hetar^{X}, Hetcyc^{X}, LA^{X};
or
R⁵ and R⁶ form together with the carbon atom to which they are attached to a saturated or partially unsaturated ring system D which ring system D is mono- or bicyclic and has 3, 4, 5, 6, 7, 8, 9, 10 ring atoms and may contain no hetero ring atom or 1, 2, 3 hetero ring atom(s) independently from each other selected from N, O and/or S that ring system D may be unsubstituted or mono-, di- or trisubstituted with independently from each other R^{D1}, R^{D2}R^{D3};
or
R⁵ and R⁶ form together with the carbon atom to which they are attached to a C═CHR^{D4} moiety;
R⁷ denotes H, Hetar^{X}, Hetcyc^{X}, LA^{X};
Ar^{X} denotes a mono-, bi- or tricyclic aromatic ring system with 5, 6, 7, 8, 9, 10, 11, 12, 13, 14 ring carbon atoms which ring system may be unsubstituted or mono-, di- or trisubstituted with independently from each other R^{X1}, R^{X2}, R^{X3};
Ar^{Y} denotes a mono-, bi- or tricyclic aromatic ring system with 5, 6, 7, 8, 9, 10, 11, 12, 13, 14 ring carbon atoms which ring system may be unsubstituted or mono-, di- or trisubstituted with independently from each other R^{Y1}, R^{Y2}, R^{Y3};
Hetar^{X} denotes a mono, bi- or tricyclic aromatic ring system with 5, 6, 7, 8, 9, 10, 11, 12, 13, 14 ring atoms wherein 1, 2, 3, 4, 5 of said ring atoms is/are a hetero atom(s) selected from N, O and/or S and the remaining are carbon atoms, wherein that aromatic ring system may be unsubstituted or mono-, di- or trisubstituted with independently from each other R^{X1}, R^{X2}, R^{X3};
Hetar^{Y} denotes a mono, bi- or tricyclic aromatic ring system with 5, 6, 7, 8, 9, 10, 11, 12, 13, 14 ring atoms wherein 1, 2, 3, 4, 5 of said ring atoms is/are a hetero atom(s) selected from N, O and/or S and the remaining are carbon atoms, wherein that aromatic ring system may be unsubstituted or mono-, di- or trisubstituted with independently from each other R^{Y1}, R^{Y2}, R^{Y3};
Hetcyc^{X} denotes a saturated or partially unsaturated mono-, bi- or tricyclic heterocycle with 3, 4, 5, 6, 7, 8, 9, 10, 11, 12, 13, 14 ring atoms wherein 1, 2, 3, 4, 5 ring atom(s) is/are heteroatom(s) selected from N, O and/or S and the remaining ring atoms are carbon atoms, wherein that heterocycle may be unsubstituted or mono-, di- or trisubstituted with R^{X4}, R^{X5}, R^{X6};
Hetcyc^{Y} denotes a saturated or partially unsaturated mono-, bi- or tricyclic heterocycle with 3, 4, 5, 6, 7, 8, 9, 10, 11, 12, 13, 14 ring atoms wherein 1, 2, 3, 4, 5 ring atom(s) is/are heteroatom(s) selected from N, O and/or S and the remaining ring atoms are carbon atoms, wherein that heterocycle may be unsubstituted or mono-, di- or trisubstituted with R^{Y4}, R^{Y5}, R^{Y6};
R^{X1}, R^{X2}, R^{X3} denote independently from each other H, Hal, LA^{X}-CN, —NO₂, —SO₂NH₂, —SO₂NHR^{X7}, —SO₂NR^{X7}R^{X8}, —NH—SO₂—R^{X9}, —NR^{X7}—SO₂—R^{X9}, —SO₂—R^{X9}, —NH₂, —NHR^{X7}, —NR^{X7}R^{X8}, —OH, —O—R^{X9}, —CHO, —C(═O)—R^{X9}, —COOH, —C(═O)—O—R^{X9}, —C(═O)—NH₂, —C(═O)—NHR^{X7}, —C(═O)—NR^{X7}R^{X8}, —NH—(C₁₋₃-alkylene)-C(═O)—NH₂, —NH—(C₁₋₃-alkylene)-C(═O)—NHR^{X7}, —NH—(C₁₋₃-alkylene)-C(═O)—NR^{X7}R^{X8}, —NH—C(═O)—R^{X9}, —NR^{X7}—C(═O)—R^{X9}
or
two of R^{X1}, R^{X2}, R^{X3} form a divalent alkylene chain with 3, 4, 5 chain carbon atoms wherein 1 or 2 of non-adjacent CH₂ groups of the divalent alkylene chain may be replaced independently from each other by —N(H)—, —N(C₁₋₆-alkyl)-, —N(—C(═O)—C₁₋₄-alkyl)-, —O— —wherein that C₁₋₆-alkyl and C₁₋₄-alkyl radicals may be straight-chain or branched—and wherein 2 adjacent CH₂ groups may together be replaced by a —CH═CH— moiety, which divalent alkylene chain may be unsubstituted or mono- or di-substituted with independently from each other straight-chain or branched —C₁₋₆-alkyl or ═O (oxo);
R^{X4}, R^{X5}, R^{X6} denote independently from each other H, Hal, LA^{X}, —CN, —NO₂, —SO₂NH₂, —SO₂NHR^{X7}, —SO₂NR^{X7}R^{X8}, —NH—SO₂—R^{X9}, —NR^{X7}—SO₂—R^{X9}, —SO₂—R^{X9}, —NH₂, —NHR^{X7}, —NR^{X7}R^{X8}, —OH, —O—R^{X9}, —CHO, —C(═O)—R^{X9}, —COOH, —C(═O)—O—R^{X9}, —C(═O)—NH₂, —C(═O)—NHR^{X7}, —C(═O)—NR^{X7}R^{X8}, —NH—(C₁₋₃-alkylene)-C(═O)—NH₂, —NH—(C₁₋₃-alkylene)-C(═O)—NHR^{X7}, —NH—(C₁₋₃-alkylene)-C(═O)—NR^{X7}R^{X8}, —NH—C(═O)—R^{X9}, —NR^{X7}—C(═O)—R^{X9}, oxo (═O);
R^{Y1}, R^{Y2}, R^{Y3} denote independently from each other H, Hal, LA^{Y}, —CN, —NO₂, —SO₂NH₂, —SO₂NHR^{Y7}, —SO₂NR^{Y7}R^{Y8}, —NH—SO₂—R^{Y9}, —NR^{Y7}—SO₂—R^{Y9}, —SO₂—R^{Y9}, —NH₂, —NHR^{Y7}, —NR^{Y7}R^{Y8}, —OH, —O—R^{Y9}, —CHO, —C(═O)—R^{Y9}, —COOH, —C(═O)—O—R^{Y9}, —C(═O)—NH₂, —C(═O)—NHR^{Y7}, —C(═O)—NR^{Y7}R^{Y8}, —NH—(C₁₋₃-alkylene)-C(═O)—NH₂, —NH—(C₁₋₃-alkylene)-C(═O)—NHR^{Y7}, —NH—(C₁₋₃-alkylene)-C(═O)—NR^{Y7}R^{Y8}, —NH—C(═O)—R^{Y9}, —NR^{Y7}—C(═O)—R^{Y9}
or
two of R^{Y1}, R^{Y2}, R^{Y3} form a divalent alkylene chain with 3, 4, 5 chain carbon atoms wherein 1 or 2 of non-adjacent CH₂ groups of the divalent alkylene chain may be replaced independently from each other by —N(H)—, —N(C₁₋₆-alkyl)-, —N(—C(═O)—C₁₋₄-alkyl)-, —O— —wherein that C₁₋₆-alkyl and C₁₋₄-alkyl radicals may be straight-chain or branched—and wherein 2 adjacent CH₂ groups may together be replaced by a —CH═CH— moiety, which divalent alkylene chain may be unsubstituted or mono- or di-substituted with independently from each other straight-chain or branched C₁₋₆-alkyl or ═O (oxo);
R^{Y4}, R^{Y5}, R^{Y6} denote independently from each other H, Hal, LA^{Y}, —CN, —NO₂, —SO₂NH₂, —SO₂NHR^{Y7}, —SO₂NR^{Y7}R^{Y8}, —NH—SO₂—R^{Y9}, —NR^{Y7}—SO₂—R^{Y9}, —SO₂—R^{Y9}, —NH₂, —NHR^{Y7}, —NR^{Y7}R^{Y8}, —OH, —O—R^{Y9}, —CHO, —C(═O)—R^{Y9}, —COOH, —C(═O)—O—R^{Y9}, —C(═O)—NH₂, —C(═O)—NHR^{Y7}, —C(═O)—NR^{Y7}R^{Y8}, —NH—(C₁₋₃-alkylene)-C(═O)—NH₂, —NH—(C₁₋₃-alkylene)-C(═O)—NHR^{Y7}, —NH—(C₁₋₃-alkylene)-C(═O)—NR^{Y7}R^{Y8}, —NH—C(═O)—R^{Y9}, —NR^{Y7}—C(═O)—R^{Y9}, oxo (═O);
LA^{X} denotes straight-chain or branched C₁₋₆-alkyl which may be unsubstituted or mono-, di- or trisubstituted with independently from each other Hal, —CN, —NO₂, —SO₂NH₂, —SO₂NHR^{X7}, —SO₂NR^{X7}R^{X8}, —NH—SO₂—R^{X9}, —NR^{X7}—SO₂—R^{X9}, —SO₂—R^{X9}, —NH₂, —NHR^{X7}, —NR^{X7}R^{X8}, —OH, —O—R^{X9}, —CHO, —C(═O)—R^{X9}, —COOH, —C(═O)—O—R^{X9}, —C(═O)—NH₂, —C(═O)—NHR^{X7}, —C(═O)—NR^{X7}R^{X8}, —NH—(C₁₋₃-alkylene)-C(═O)—NH₂, —NH—(C₁₋₃-alkylene)-C(═O)—NHR^{X7}, —NH—(C₁₋₃-alkylene)-C(═O)—NR^{X7}R^{X8}, —NH—C(═O)—R^{X9}, —NR^{X7}—C(═O)—R^{X9}, oxo (═O), wherein 1 or 2 non-adjacent CH₂ groups of the C₁₋₆-alkyl radical may independently from each other be replaced by O, S, N(H) or N—R^{X7} and/or 1 or 2 non-adjacent CH groups of the C₁₋₆-alkyl radical may independently from each other be replaced by N;
LA^{Y} denotes straight-chain or branched C₁₋₆-alkyl which may be unsubstituted or mono-, di- or trisubstituted with independently from each other Hal, —CN, —NO₂, —SO₂NH₂, —SO₂NHR^{Y7}, —SO₂NR^{Y7}R^{Y8}, —NH—SO₂—R^{Y9}, —NR^{Y7}—SO₂—R^{Y9}, —SO₂—R^{Y9}, —NH₂, —NHR^{Y7}, —NR^{Y7}R^{Y8}, —OH, —O—R^{Y9}, —CHO, —C(═O)—R^{Y9}, —COOH, —C(═O)—O—R^{Y9}, —C(═O)—NH₂, —C(═O)—NHR^{Y7}, —C(═O)—NR^{Y7}R^{Y8}, —NH—(C₁₋₃-alkylene)-C(═O)—NH₂, —NH—(C₁₋₃-alkylene)-C(═O)—NHR^{Y7}, —NH—(C₁₋₃-alkylene)-C(═O)—NR^{Y7}R^{Y8}, —NH—C(═O)—R^{Y9}, —NR^{Y7}—C(═O)—R^{Y9}, oxo (═O), wherein 1 or 2 non-adjacent CH₂ groups of the alkyl chain may independently from each other be replaced by O, S, N(H) or N—R^{Y7} and/or 1 or 2 non-adjacent CH groups of the alkyl chain may independently from each other be replaced by N;
LA^{Z} denotes a divalent straight-chain or branched C₁₋₆-alkylene radical which divalent alkylene radical may be unsubstituted or mono-, di- or trisubstituted with independently from each other Hal, —CN, —NO₂, —SO₂NH₂, —SO₂NHR^{Z7}, —SO₂NR^{Z7}R^{Z8}, —NH—SO₂—R^{Z9}, —NR^{Z7}—SO₂—R^{Z9}, —SO₂—R^{Z9}, —NH₂, —NHR^{Z7}, —NR^{Z7}R^{Z8}, —OH, —O—R^{Z9}, —CHO, —C(═O)—R^{Z9}, —COOH, —C(═O)—O—R^{Z9}, —C(═O)—NH₂, —C(═O)—NHR^{Z7}, —C(═O)—NR^{Z7}R^{Z8}, —NH—(C₁₋₃-alkylene)-C(═O)—NH₂, —NH—(C₁₋₃-alkylene)-C(═O)—NHR^{Z7}, —NH—(C₁₋₃-alkylene)-C(═O)—NR^{Z7}R^{Z8}, —NH—C(═O)—R^{Z9}, —NR^{Z7}—C(═O)—R^{Z9}, oxo (═O), wherein 1 or 2 non-adjacent CH₂ groups of the divalent alkylene radical may be replaced independently from each other by O, S or N(H) and/or 1 or 2 non-adjacent CH groups of the divalent alkylene radical may be replaced by N;
CA^{X} denotes a saturated monocyclic carbocycle with 3, 4, 5, 6, 7 carbon atoms which carbocycle may be unsubstituted or mono- or disubstituted with independently from each other R^{CA1}, R^{CA2};
R^{X7}, R^{X8}, R^{Y7}, R^{Y8}R^{Z7}, R^{Z8} denote independently from each other straight-chain or branched C₁₋₆-alkyl, phenyl, a monocyclic aromatic ring system with 5, 6, 7 ring atoms wherein 1, 2, 3, 4 of said ring atoms is/are a hetero atom(s) selected from N, O and/or S and the remaining are carbon atoms and wherein that aromatic ring system may be unsubstituted or mono- or disubstituted with independently from each other straight-chain or branched C₁₋₆-alkyl, or a saturated monocyclic carbocycle with 3, 4, 5, 6, 7 carbon atoms
or
each pair R^{X7} and R^{X8}; R^{Y7} and R^{Y8}; R^{Z7} and R^{Z8} form independently from each other pair together with the nitrogen atom to which they are attached to a 3, 4, 5, 6 or 7 membered heterocycle wherein that heterocycle may not contain any further heteroatom or may contain besides said nitrogen atom one further hetero ring atom selected from N, O and S, wherein, if that further hetero atom is N, that further N may be substituted with H or straight-chain or branched C₁₋₆-alkyl;
R^{X9}, R^{Y9}, R^{Z9} denote independently from each other straight-chain or branched —C₁₋₆-alkyl, which may be unsubstituted or mono-, di- or trisubstituted with Hal, phenyl, a monocyclic aromatic ring system with 5, 6, 7 ring atoms wherein 1, 2, 3, 4 of said ring atoms is/are a hetero atom(s) selected from N, O and/or S and the remaining are carbon atoms and wherein that aromatic ring system may be unsubstituted or mono- or disubstituted with independently from each other straight-chain or branched C₁₋₆-alkyl, or a saturated monocyclic carbocycle with 3, 4, 5, 6, 7 carbon atoms;
R^{A1}, R^{A2}, R^{A3} denote independently from each other H, Hal, LA^{X}, Ar^{X}, Hetar^{X}, —CN, —NO₂, —SO₂NH₂, —SO₂NHR^{X7}, —SO₂NR^{X7}R^{X8}, —NH—SO₂—R^{X9}, —NR^{X7}—SO₂—R^{X9}, —SO₂—R^{X9}, —NH₂, —NHR^{X7}, —NR^{X7}R^{X8}, —OH, —O—R^{X9}, —CHO, —C(═O)—R^{X9}, —COOH, —C(═O)—O—R^{X9}, —C(═O)—NH₂, —C(═O)—NHR^{X7}, —C(═O)—NR^{X7}R^{X8}, —NH—(C₁₋₃-alkylene)-C(═O)—NH₂, —NH—(C₁₋₃-alkylene)-C(═O)—NHR^{X7}, —NH—(C₁₋₃-alkylene)-C(═O)—NR^{X7}R^{X8}, —NH—C(═O)—R^{X9}, —NR^{X7}—C(═O)—R^{X9}, oxo (═O);
or
two of R^{A1}, R^{A2} and R^{A3} form together with one carbon atom of that ring system A to which they both are attached to a saturated or partially unsaturated ring system E which ring system E is mono- or bicyclic and has 3, 4, 5, 6, 7, 8, 9, 10 ring atoms and may contain no hetero atom or 1, 2, 3 hetero atom(s) independently from each other selected from N, O and/or S that ring system E may be unsubstituted or mono-, di- or trisubstituted with independently from each other R^{E1}, R^{E2}, R^{E3};
R^{D1}, R^{D2}, R^{D3}, R^{E1}, R^{E2}, R^{E3} denote independently from each other H, Hal, LA^{X}, —CN, —NO₂, —SO₂NH₂, —SO₂NHR^{X7}, —SO₂NR^{X7}R^{X8}, —NH—SO₂—R^{X9}, —NR^{X7}—SO₂—R^{X9}, —SO₂—R^{X9}, —NH₂, —NHR^{X7}, —NR^{X7}R^{X8}, —OH, —O—R^{X9}, —CHO, —C(═O)—R^{X9}, —COOH, —C(═O)—O—R^{X9}, —C(═O)—NH₂, —C(═O)—NHR^{X7}, —C(═O)—NR^{X7}R^{X8}, —NH—(C₁₋₃-alkylene)-C(═O)—NH₂, —NH—(C₁₋₃-alkylene)-C(═O)—NHR^{X7}, —NH—(C₁₋₃-alkylene)-C(═O)—NR^{X7}R^{X8}, —NH—C(═O)—R^{X9}, —NR^{X7}—C(═O)—R^{X9}, oxo (═O);
R^{D4} denotes —COOH;
R^{CA1}, R^{CA2} denote independently from each other H, Hal, R^{X9}, —CN, —NO₂, —SO₂NH₂, —SO₂NHR^{X7}, —SO₂NR^{X7}R^{X8}, —NH—SO₂—R^{X9}, —NR^{X7}—SO₂—R^{X9}, —S—R^{X9}, S(═O)—R^{X9}, —SO₂—R^{X9}, —NH₂, —NHR^{X7}, —NR^{X7}R^{X8}, —OH, —O—R^{X9}, —CHO, —C(═O)—R^{X9}, —COOH, —C(═O)—O—R^{X9}, —C(═O)—NH₂, —C(═O)—NHR^{X7}, —C(═O)—NR^{X7}R^{X8}, —NH—(C₁₋₃-alkylene)-C(═O)—NH₂, —NH—(C₁₋₃-alkylene)-C(═O)—NHR^{X7}, —NH—(C₁₋₃-alkylene)-C(═O)—NR^{X7}R^{X8}, —NH—C(═O)—R^{X9}, —NR^{X7}—C(═O)—R^{X9}, oxo (═O);
Hal denotes F, Cl, Br, I;
or derivatives, N-oxides, prodrugs, solvates, tautomers or stereoisomers thereof as well as the physiologically acceptable salts of each of the foregoing, including mixtures thereof in all ratios.

3. Compound according to any one of claims 1 or 2, or derivatives, N-oxides, prodrugs, solvates, tautomers or stereoisomers thereof as well as the physiologically acceptable salts of each of the foregoing, including mixtures thereof in all ratios,
wherein
R² denotes H, unsubstituted straight-chain or branched —C₁₋₆-alkyl, —OH, —CN;
R³ denotes H, unsubstituted straight-chain or branched —C₁₋₆-alkyl, —OH.

4. Compound according to claim 3, or derivatives, N-oxides, prodrugs, solvates, tautomers or stereoisomers thereof as well as the physiologically acceptable salts of each of the foregoing, including mixtures thereof in all ratios,
wherein
R² denotes H;
R³ denotes H.

5. Compound according to any one of claims 1 to 4, or derivatives, N-oxides, prodrugs, solvates, tautomers or stereoisomers thereof as well as the physiologically acceptable salts of each of the foregoing, including mixtures thereof in all ratios,
wherein
X denotes N—R⁷ or O;
R⁷ denotes H or straight-chain or branched —C₁₋₆-alkyl or Hetar^{X}.

6. Compound according to claim 5, or derivatives, N-oxides, prodrugs, solvates, tautomers or stereoisomers thereof as well as the physiologically acceptable salts of each of the foregoing, including mixtures thereof in all ratios,
wherein
X denotes N—R⁷;
R⁷ denotes H or Hetar^{X7}, preferably H;
Hetar^{X7} denotes a monocyclic aromatic ring system with 5, 6, 7 ring atoms wherein 1, 2, 3, 4 of said ring atoms is/are a hetero atom(s) selected from N, O and/or S and the remaining are carbon atoms, wherein that aromatic ring system may be unsubstituted or mono- or disubstituted with independently from each other R^{X71a}, R^{X72a}; preferably triazolyl or pyridinyl, each of which is unsubstituted or monosubstituted with R^{X79}, —C(═O)—NH₂, —SO₂—R^{X79};
R^{X71a}, R^{X72a} denotes independently from each other Hal, R^{X79}, N, —NO₂, —SO₂NH₂, —SO₂NHR^{X77}, —SO₂NR^{X77}R^{X78}, —NH—SO₂—R^{X79}, —NR^{X77}—SO₂—R^{X79}, —SO₂—R^{X79}, —NH₂, —NHR^{X77}, —NR^{X77}R^{X78}, —OH, —O—R^{X79}, —CHO, —C(═O)—R^{X79}, —COOH, —C(═O)—O—R^{X79}, —C(═O)—NH₂, —C(═O)—NHR^{X77}, —C(═O)—NR^{X77}R^{X78}, —NH—C(═O)—R^{X79}, —NR^{X77}—C(═O)—R^{X79};
R^{X77}, R^{X78}, R^{X79} denote independently from each other straight-chain or branched C₁₋₆-alkyl or a saturated monocyclic carbocycle with 3, 4, 5, 6, 7 carbon atoms
or
R^{X77} and R^{X78} form together with the nitrogen atom to which they are attached to a 3, 4, 5, 6 or 7 membered heterocycle wherein that heterocycle may not contain any further heteroatom or may contain besides said nitrogen atom one further hetero ring atom selected from N, O and S, wherein, if that further hetero atom is N, that further N may be substituted with H or straight-chain or branched C₁₋₆-alkyl.

7. Compound according to any one of claims 1 to 6, or derivatives, N-oxides, prodrugs, solvates, tautomers or stereoisomers thereof as well as the physiologically acceptable salts of each of the foregoing, including mixtures thereof in all ratios,
wherein
R¹ denotes Ar^{X}, Hetar^{X} or Hetar^{X}-LA^{Z}-Ar^{Y}.

8. Compound according to any one of claims 1 to 7, or derivatives, N-oxides, prodrugs, solvates, tautomers or stereoisomers thereof as well as the physiologically acceptable salts of each of the foregoing, including mixtures thereof in all ratios,
wherein
R¹ denotes Ar^{X1}, Hetar^{X1} or Hetar^{X1}-LA^{Z1}-Ar^{Y1};
Ar^{X1} denotes a mono- or bicyclic aromatic ring system with 6, 7, 8, 9, 10 ring carbon atoms which ring system may be unsubstituted or mono-, di- or trisubstituted with independently from each other R^{X1a}, R^{X2a}, R^{X3a};
Ar^{Y1} denotes a mono- or bicyclic aromatic ring system with 6, 7, 8, 9, 10 ring carbon atoms which ring system may be unsubstituted or mono-, di- or trisubstituted with independently from each other R^{Y1a}, R^{Y2a}, R^{Y3a};
Hetar^{X1} denotes a mono or bicyclic aromatic ring system with 5, 6, 8, 9, 10 ring atoms wherein 1, 2, 3 of said ring atoms is/are a hetero atom(s) selected from N, O and/or S and the remaining are carbon atoms, wherein that aromatic ring system may be unsubstituted or mono-, di- or trisubstituted with independently from each other R^{X1b}, R^{X2b}, R^{X3b};
LA^{Z1} denotes a divalent straight-chain or branched C₁₋₆-alkylene radical;
R^{X1a}, R^{X2a}, R^{X3a}, R^{X1b}, R^{X2b}, R^{X3b}, R^{Y1a}, R^{Y2a}, R^{Y3a} denote independently from each other LA^{X1}, Br, —CN, —C(═O)—NH₂, —C(═O)—R^{X9a}, —NH₂, —NHR^{X7a}, —NR^{X7a}R^{X8a}, —NO₂, —OR^{X9a}
or
two of R^{X1a}, R^{X2a}, R^{X3a} form a divalent alkylene chain with 3, 4 or 5 chain carbon atoms wherein 1 or 2 of non-adjacent CH₂ groups of the divalent alkylene chain may be replaced independently from each other by —N(H)—, —N(C₁₋₆-alkyl)-, —N(—C(═O)—C₁₋₄-alkyl)-, —O— —wherein that C₁₋₆-alkyl and C₁₋₄-alkyl radicals may be straight-chain or branched—and wherein 2 adjacent CH₂ groups may together be replaced by a —CH═CH— moiety, which divalent alkylene chain may be unsubstituted or mono- or di-substituted with straight-chain or branched C₁₋₆-alkyl and/or mono-substituted with ═O (oxo);
LA^{X1} denotes straight-chain or branched —C₁₋₆-alkyl which may be unsubstituted or mono-substituted with —OR^{X9a};
R^{X7a}, R^{X8a} denote independently from each other straight-chain or branched —C₁₋₆-alkyl or form together with the nitrogen atom to which they are attached to a 3, 4, 5, 6 or 7 membered heterocycle wherein that heterocycle may not contain any further heteroatom or may contain besides said nitrogen atom one further hetero ring atom selected from N, O and S, wherein, if that further hetero atom is N, that further N may be substituted with H or straight-chain or branched —C₁₋₆-alkyl;
R^{X9a} denotes straight-chain or branched —C₁₋₆-alkyl.

9. Compound according to any one of claims 1 to 8, or derivatives, N-oxides, prodrugs, solvates, tautomers or stereoisomers thereof as well as the physiologically acceptable salts of each of the foregoing, including mixtures thereof in all ratios,
wherein
R¹ denotes Ar^{X1}, Hetar^{X1} or Hetar^{X1}-LA^{Z1}-Ar^{Y1};
Ar^{X1} denotes phenyl or naphthyl which may be unsubstituted or mono- or disubstituted with R^{X1a}, R^{X2a};
Hetar^{X1} denotes (a) a monocyclic aromatic ring system with 6 ring atoms wherein 1 of said ring atoms is a nitrogen atom and the remaining are carbon atoms; or (b) a bicyclic aromatic ring system with 9 ring atoms wherein (i) 1 of said ring atoms is a nitrogen atom or an oxygen atom or a sulfur atom and the remaining are carbon atoms; or (ii) 2 of said ring atoms are nitrogen atoms and the remaining are carbon atoms; or (iii) 1 of said ring atoms is a nitrogen atom and 1 of said ring atoms is a sulfur atom and the remaining ring atoms are carbon atoms, wherein that mono- or bicyclic aromatic ring system may be unsubstituted or mono-substituted with straight-chain or branched C₁₋₄-alkyl or R^{X1b} or disubstituted with independently from each other straight-chain or branched C₁₋₄-alkyl;
Ar^{Y1} denotes phenyl;
LA^{Z1} denotes a divalent straight-chain or branched C₁₋₄-alkylene radical, preferably CH₂;
R^{X1a}, R^{X2a} denote independently from each other straight-chain or branched —C₁₋₆-alkyl or —O—C₁₋₆-alkyl, —NH₂, —NHR^{X7a}, —NR^{X7a}R^{X8a} or form together a divalent alkylene chain with 3, 4, 5 chain carbon atoms wherein 1 or 2 of non-adjacent CH₂ groups of the divalent alkylene chain may be replaced independently from each other by —N(H)—, —N(C₁₋₆-alkyl)-, —N(—C(═O)—C₁₋₄-alkyl)-, —O— —wherein that C₁₋₆-alkyl and C₁₋₄-alkyl radicals may be straight-chain or branched—, which divalent alkylene chain may be unsubstituted or mono- or di-substituted with independently from each other straight-chain or branched C₁₋₆-alkyl or ═O (oxo);
R^{X1b} denotes —O-methyl, —NH₂, —C(═O)-methyl;
R^{X7a}, R^{X8a} denote independently from each other straight-chain or branched —C₁₋₄-alkyl.

10. Compound according to claim 9, or derivatives, N-oxides, prodrugs, solvates, tautomers or stereoisomers thereof as well as the physiologically acceptable salts of each of the foregoing, including mixtures thereof in all ratios,
wherein
Ar^{X1} denotes 3-(methylamino)-4-methylphenyl, 3-(dimethylamino)-4-methylphenyl, 3-(dimethylamino)-4-methoxyphenyl, 1-methyl-2,3-dihydro-1H-indol-6-yl (phenyl with R^{X1a} in 3-position and R^{X2a} in 4-position, R^{X1a} and R^{X2a} forming together a —N(CH₃)—CH₂—CH₂-chain), 1-methyl-1,2,3,4-tetrahydroquinolin-7-yl (phenyl with R^{X1a} in 3-position and R^{X2a} in 4-position, R^{X1a} and R^{X2a} forming together a —N(CH₃)—CH₂—CH₂—CH₂— chain), 4-methyl-1,2,3,4-tetrahydroquinoxalin-6-yl (phenyl with with R^{X1a} in 3-position and R^{X2a} in 4-position, R^{X1a} and R^{X2a} forming together a —N(CH₃)—CH₂—CH₂—NH— chain), 5-methyl-2,3,4,5-tetrahydro-1H-1,5-benzodiazepin-7-yl, naphthyl;
Hetar^{X1} denotes 1H-indol-6-yl, N-methyl-indol-6-yl (1-methyl-1H-indol-6-yl), 1-methyl-1H-indol-5-yl, 3-methyl-1H-indol-5-yl, 1,3-dimethyl-1H-indol-5-yl, 1-ethyl-1H-indol-6-yl, 1-ethyl-1H-indol-5-yl, 3-methyl-1-benzofuran-5-yl, 3-methyl-1-benzothiophen-5-yl, 1-methyl-1H-indazol-6-yl, 2-amino-1,3-benzothiazol-5-yl, 1-methyl-1H-pyrrolo[2,3-b]pyrdin-6-yl.

11. Compound according to any one of claims 1 to 10, or derivatives, N-oxides, prodrugs, solvates, tautomers or stereoisomers thereof as well as the physiologically acceptable salts of each of the foregoing, including mixtures thereof in all ratios,
wherein
R⁵ and R⁶ both denote H.

12. Compound according to claim 11, or derivatives, N-oxides, prodrugs, solvates, tautomers or stereoisomers thereof as well as the physiologically acceptable salts of each of the foregoing, including mixtures thereof in all ratios,
wherein
R⁴ denotes Ar^{X}, Ar^{X}-Hetar^{Y}, Ar^{X}-Hetcyc^{Y}, Hetar^{X}, Hetar^{X}-Hetar^{Y}, Hetar^{X}-Hetcyc^{Y}, Hetcyc^{X}, Hetcyc^{X}-Hetar^{Y}, LA^{Z}-Hetar^{Y}.

13. Compound according to any one of claims 11 to 12, or derivatives, N-oxides, prodrugs, solvates, tautomers or stereoisomers thereof as well as the physiologically acceptable salts of each of the foregoing, including mixtures thereof in all ratios,
wherein
R⁴ denotes Ar^{X4}, Ar^{X4}-Hetar^{Y4}, Hetar^{X4}, Hetar^{X4}-Hetar^{Y4}, Hetar^{X4}-Hetcyc^{Y4}, Hetcyc^{X4}, LA^{Z4}-Hetar^{Y4};
Ar^{X4} denotes phenyl which may be unsubstituted or mono- or di-substituted with independently from each other R^{X1c}, R^{X2c};
Hetar^{X4} denotes a mono- or bicyclic aromatic ring system with 5, 6, 8, 9, 10 ring atoms wherein 1, 2, 3 of said ring atoms is/are a hetero atom(s) selected from N, O and/or S and the remaining are carbon atoms, wherein that aromatic ring system may be unsubstituted or mono- or di-substituted with independently from each other R^{X1d}, R^{X2d};
Hetcyc^{X4} denotes a saturated or partially unsaturated mono-cyclic heterocycle with 3, 4, 5, 6, 7 ring atoms wherein
(i) 1 ring atom is a heteroatom selected from N, O; or (ii) 1 ring atom is N and 1 ring atom is O; or (iii) 2 ring atoms are N;
and the remaining ring atoms are carbon atoms,
wherein that heterocycle may be unsubstituted or mono-substituted with R^{X4a};
Hetar^{Y4} denotes a monocyclic aromatic ring system with 5 or 6 ring atoms wherein 1, 2, 3, 4 of said ring atoms are N and the remaining are carbon atoms. wherein that aromatic ring system may be unsubstituted or mono-substituted with R^{Y4a};
Hetcyc^{Y4} denotes a saturated or partially unsaturated mono-cyclic heterocycle with 3, 4, 5, 6, 7 ring atoms wherein
(i) 1 ring atom is a heteroatom selected from N, O; or (ii) 1 ring atom is N and 1 ring atom is O; or (iii) 2 ring atoms are N;
and the remaining ring atoms are carbon atoms,
wherein that heterocycle may be unsubstituted or mono-substituted with R^{Y4b};
LA^{Z4} denotes a divalent straight-chain or branched —C₁₋₆-alkylene radical;
R^{X1c}, R^{X2c}, R^{X1d}, R^{X2d} denote independently from each other Hal, R^{X9b}, —CN, —NO₂, —SO₂NH₂, SO₂—R^{X9b}, —NH₂, —OH, —O—R^{X9b}, —C(═O)—NH₂
or
R^{X1d} and R^{X2d} form a divalent alkylene chain with 3 or 4 carbon atoms wherein 1 or 2 of non-adjacent CH₂ groups of the divalent alkylene chain may be replaced independently from each other by —N(H)—, —O— which divalent alkylene chain may be unsubstituted or mono-substituted with ═O (oxo);
R^{X4a} denotes ═O (oxo), straight-chain or branched —C₁₋₆-alkyl;
R^{X9b} denotes straight-chain or branched —C₁₋₆-alkyl;
R^{Y4a} denotes NH₂, straight-chain or branched —C₁₋₆-alkyl;
R^{Y4b} denotes straight-chain or branched —C₁₋₆-alkyl, —C(═O)—R^{X9b};
Hal denotes F, Cl, Br, I.

14. Compound according to any one of claims 11 to 13, or derivatives, N-oxides, prodrugs, solvates, tautomers or stereoisomers thereof as well as the physiologically acceptable salts of each of the foregoing, including mixtures thereof in all ratios,
wherein
R⁴ denotes pyrdin-3-yl-methyl, pyridinyl, oxanyl, thiazol-4-yl, thiazol-5-yl, 1,2-thiazolyl, 1,3-thiazolyl, methylthiazolyl, 3-methyl-1,2-thiazol-5-yl, 5-(1-methyl-1H-pyrazol-4-yl)pyridin-3-yl, 4-benzonitrile, 3-benzonitrile, 1-methyl-1H-imidazol-5-yl, dimethylimidazolyl, 1,2-dimethyl-1H-imidazol-5-yl, triazolyl, 4H-1,2,4-triazol-3-yl, methyltriazolyl, 4-methyl-4H-1,2,4-triazol-3-yl, 1-methyl-1H-1,2,3-triazol-5-yl, 5-methyl-1H-1,2,4-triazol-3-yl, oxazolyl (1,3-oxazolyl), methyloxazolyl, 2-methyl-1,3-oxazol-5-yl, isoxazolyl (1,2-oxazolyl), methyloxadiazolyl, 2-methyl-1,3,4-oxydiazol-5-yl, 5-(1H-imidazol-1-yl)pyridin-3-yl, 5-(2-aminopyrimidin-5-yl)pyridin-3-yl, 5-(1H-pyrazol-4-yl)pyridin-3-yl, 4-(1-methyl-1H-pyrazol-4-yl)pyridin-2-yl, 2-(1-methyl-1H-pyrazol-4-yl)pyridin-4-yl, 4-(1H-1,2,3,4-tetrazol-5-yl)phenyl, 3-(1H-1,2,3,4-tetrazol-5-yl)phenyl, 3-benzamide, 3-aminophenyl, phenyl, furan-2-yl, piperindin-3-yl, morpholin-2-yl, 1H-pyrazol-4-yl, methylpyrazolyl, 1-methyl-1H-pyrazol-5-yl, 1-methyl-1H-pyrazol-4-yl, 2-methansulfonylphenyl, 4-methansulfonylphenyl, 3-methansulfonylphenyl, piperidin-2-yl, pyridazin-3-yl, pyridazin-4-yl, methoxypyridinyl, 4-methoxypyridin-3-yl, 4-bromo-pyridin-2-yl, 2-bromopyridin-4-yl, 5-bromopyridin-3-yl, cyanopyridinyl, 4-cyanopyridin-3-yl, 5-(pyrimidin-5-yl)pyridin-3-yl, aminopyridinyl, 5-aminopyridin-3-yl, 4-amino-pyridin-3-yl, 5-(1H-pyrazol-5-yl)pyridin-3-yl, N-acetylpiperazinyl-pyridinyl, 4-(4-acetylpiperazin-1-yl)pyridin-3-yl, acetylmorpholinyl, pyrazolylpyridin-3-yl, imidazopyridinyl, methylpiperazinylpyridinyl, pyrimidinylpyridinyl, methylmorpholinyl, pyrimidinyl, chloropyrimidinyl, aminopyrimidinyl, acetylpiperidinyl, pyridinonyl (hydroxypyridinyl), methylpiperidinyl, hydroxypyridinyl, fluoropyridinyl, methylpyridinyl, methoxypyridinyl, morpholinylpyridinyl.

15. Compound according to any one of claims 11 to 14, or derivatives, N-oxides, prodrugs, solvates, tautomers or stereoisomers thereof as well as the physiologically acceptable salts of each of the foregoing, including mixtures thereof in all ratios,
R⁴ denotes pyridin-3-yl, 3-bromopyridin-3-yl, oxan-3-yl, 1,2-thiazol-4-yl, 1,2-thiazol-5-yl, 1,3-thiazol-5-yl, 1-methyl-1H-imidazol-5-yl, 4H-1,2,4-triazol-3-yl, 1-methyl-1H-1,2,3-triazol-5-yl, 1,2-oxazol-4-yl, 1,3-oxazol-5-yl, 5-(1-methyl-1H-pyrazol-4-yl)pyridin-3-yl, 5-(1H-imidazol-1-yl)pyridin-3-yl, 5-(2-aminopyrimidin-5-yl)pyridin-3-yl, 5-(1H-pyrazol-4-yl)pyridin-3-yl, morpholin-2-yl, piperidin-2-yl, 4-acetylmorpholin-2-yl, methylpyrazolylpyridin-3-yl, 4-(1-methyl-1H-pyrazol-4-yl)-pyridin-3-yl, imidazo[1,2-a]pyridin-6-yl, 4-(4-methylpiperazinyl)pyridin-3-yl, 4-(pyrimidin-5-yl)pyridin-3-yl, 4-(4-acetylpiperazin-1-yl)pyridin-3-yl, 4-methylmorpholin-2-yl, 4-methoxypyridin-3-yl, 2-chloro-pyrimidin-5-yl, 5-bromopyridin-3-yl, 2-aminopyrimidin-5-yl, N-acetylpiperidin-2-yl, 1,2-dihydropyridin-2-on-5-yl (2-hydroxypyridin-5-yl), N-methylpiperidin-2-yl, 3-hydroxypyridinyl, 4-fluoropyridin-3-yl, 4-methylpyridin-3-yl, 3-N-morpholinylpyridin-5-yl.

16. Compound according to any one of claims 1 to 10, or derivatives, N-oxides, prodrugs, solvates, tautomers or stereoisomers thereof as well as the physiologically acceptable salts of each of the foregoing, including mixtures thereof in all ratios,
wherein
R⁵ denotes Hetar^{X}, Hetcyc^{X}, LA^{X}, CA^{X};
R⁶ denotes H.

17. Compound according to claim 16, or derivatives, N-oxides, prodrugs, solvates, tautomers or stereoisomers thereof as well as the physiologically acceptable salts of each of the foregoing, including mixtures thereof in all ratios,
wherein
R⁵ denotes Hetar^{X5}, Hetcyc^{X5}, LA^{X5}, CA^{X5}
Hetar^{X5} denotes a mono- or bicyclic aromatic ring system with 5, 6, 8, 9, 10 ring atoms wherein 1, 2, 3, 4 of said ring atoms is/are a hetero atom(s) selected from N, O and/or S and the remaining are carbon atoms, wherein that aromatic ring system may be unsubstituted or mono- or di-substituted with independently from each other R^{X1e}, R^{X2e};
Hetcyc^{X5} denotes a saturated monocyclic heterocycle with 3, 4, 5, 6, 7 ring atoms wherein 1 or 2 ring atom(s) is/are heteroatom(s) selected from N and/or O and the remaining ring atoms are carbon atoms, wherein that heterocycle may be unsubstituted or mono-substituted with R^{X4a};
LA^{X5} denotes straight-chain or branched —C₁₋₆-alkyl which may be unsubstituted or mono-, di- or trisubstituted with independently from each other Hal or —CN, or mono-substituted with —C(═O)—R^{X9c}, —COOH, —C(═O)—O—R^{X9c}, —C(═O)—NH₂, —C(═O)—NHR^{X7c}, —C(═O)—NR^{X7c}R^{X8c};
CA^{X5} denotes a saturated monocyclic carbocycle with 3, 4, 5, 6, 7 carbon atoms which carbocycle may be unsubstituted or monosubstituted with —OH, —NH₂, —NH—C(═O)—R^{X9c};
R^{X1e}, R^{X2e} denote independently from each other Hal, R^{X9c}, —CN, —NO₂, —SO₂NH₂, —SO₂—R^{X9c}, —NH₂, —NHR^{X7c}, —NR^{X7c}R^{X8c}, —OH, —O—R^{X9c}, —C(═O)—NH₂;
R^{X4a} denotes H, LA^{X5a}, Hal, R^{X9c}, —SO₂—R^{X9c}, —CHO, —C(═O)—R^{X9c}, —COOH, —C(═O)—O—R^{X9c}, —C(═O)—NH₂, —C(═O)—NHR^{X7c}, —C(═O)—NR^{X7c}R^{X8c}, oxo (═O);
LA^{X5a} denotes straight-chain or branched —C₁₋₆-alkyl which may be unsubstituted or mono-, di- or trisubstituted with independently from each other Hal or mono- or disubstituted with independently from each other Hal, —CN, oxo,
O—R^{X9c}, —NH₂, —NHR^{X7c}, —NR^{X7c}R^{X8c}, —COOH, —C(═O)—O—R^{X9c}, —C(═O)—NH₂, —C(═O)—NHR^{X7c}, —C(═O)—NR^{X7c}R^{X8c} or —C(═O)—R^{X9c};
R^{X7c}, R^{X8c} denote independently from each other straight-chain or branched —C₁₋₆-alkyl or form together with the nitrogen atom to which they are attached to a 3, 4, 5, 6 or 7 membered heterocycle wherein that heterocycle may not contain any further heteroatom or may contain besides said nitrogen atom one further hetero ring atom selected from N, O and S, wherein, if that further hetero atom is N, that further N may be substituted with H or straight-chain or branched —C₁₋₆-alkyl;
R^{X9c} denotes straight-chain or branched —C₁₋₆-alkyl or a saturated monocyclic carbocycle with 3, 4, 5, 6, 7 carbon atoms;
Hal denotes F, Cl, Br, I.

18. Compound according to any of claims 16 or 17, or derivatives, N-oxides, prodrugs, solvates, tautomers or stereoisomers thereof as well as the physiologically acceptable salts of each of the foregoing, including mixtures thereof in all ratios,
wherein
R⁵ denotes LA^{X5}, CA^{X5}, Hetar^{X5} or Hetcyc^{X5};
Hetar^{X5} denotes a monocyclic aromatic ring system with 5 or 6 ring atoms wherein 1, 2, 3 or 4 of said ring atoms is/are a nitrogen atom(s), 0 or 1 of said ring atoms is an oxygen or a sulfur atom and the remaining are carbon atoms, wherein that aromatic ring system may be unsubstituted or monosubstituted with R^{X1e};
Hetcyc^{X5} denotes a saturated monocyclic heterocycle with 3, 4, 5, 6, 7 ring atoms wherein 1 or 2 ring atom(s) is/are heteroatom(s) selected from N and/or O and the remaining ring atoms are carbon atoms, wherein that heterocycle may be unsubstituted or mono-substituted with R^{X4a};
LA^{X5} denotes straight-chain or branched —C₁₋₆-alkyl which may be unsubstituted or monosubstituted with —C(═O)—NH₂, —C(═O)—NHR^{X7c}, —C(═O)—NR^{X7c}R^{X8c};
CA^{X5} denotes a saturated monocyclic carbocycle with 3, 4, 5, 6, 7 carbon atoms which carbocycle may be unsubstituted or monosubstituted with —OH, —NH₂, —NH—C(═O)—R^{X9c};
R^{X1e} denotes R^{X9c};
R^{X4a} denotes H, LA^{X5a}, R^{X9c}, —SO₂—R^{X9c}, —C(═O)—R^{X9c}, —C(═O)—NHR^{X7c}, —C(═O)—NR^{X7c}R^{X8c}, oxo (═O);
LA^{X5a} denotes straight-chain or branched —C₁₋₆-alkyl which may be unsubstituted or monosubstituted with —CN, oxo, —COOH, —C(═O)—NH₂, —C(═O)—NHR^{X7c}, —C(═O)—NR^{X7c}R^{X8c} or —C(═O)—R^{X9c} or disubstituted with oxo and —O—R^{X9c} or —NH₂;
R^{X7c}, R^{X8c} denote independently from each other straight-chain or branched —C₁₋₆-alkyl or form together with the nitrogen atom to which they are attached to a 3, 4, 5, 6 or 7 membered heterocycle wherein that heterocycle may not contain any further heteroatom or may contain besides said nitrogen atom one further hetero ring atom selected from N, O and S, wherein, if that further hetero atom is N, that further N may be substituted with H or straight-chain or branched —C₁₋₆-alkyl;
R^{X9c} denotes straight-chain or branched —C₁₋₆-alkyl or a saturated monocyclic carbocycle with 3, 4, 5, 6, 7 carbon atoms.

19. Compound according to any one of claims 16 to 18, or derivatives, N-oxides, prodrugs, solvates, tautomers or stereoisomers thereof as well as the physiologically acceptable salts of each of the foregoing, including mixtures thereof in all ratios,
wherein
R⁴ denotes Ar^{X}, Ar^{X}-Hetar^{Y}, Hetar^{X}, Hetar^{X}-Hetar^{Y}, Hetar^{X}-Hetcyc^{Y}, LA^{Z}-Hetcyc^{Y} or Hetcyc^{X}.

20. Compound according to any one of claims 16 to 19, or derivatives, N-oxides, prodrugs, solvates, tautomers or stereoisomers thereof as well as the physiologically acceptable salts of each of the foregoing, including mixtures thereof in all ratios,
wherein
R⁴ denotes Ar^{X4}, Ar^{X4}-Hetar^{Y4}, Hetar^{X4}, Hetar^{X4}-Hetar^{Y4}, Hetar^{X4}-Hetcyc^{Y4}, Hetcyc^{X4};
Ar^{X4} denotes phenyl which may be unsubstituted or mono- or di-substituted with independently from each other R^{X1f}, R^{X2f};
Hetar^{X4} denotes a mono- or bicyclic aromatic ring system with 5, 6, 8, 9, 10 ring atoms wherein 1, 2, 3 of said ring atoms is/are a hetero atom(s) selected from N, O and/or S and the remaining are carbon atoms, wherein that aromatic ring system may be unsubstituted or mono- or di-substituted with independently from each other R^{X1g}, R^{X2g};
Hetar^{Y4} denotes a monocyclic aromatic ring system with 5, 6 ring atoms wherein 1, 2, 3, 4 of said ring atoms are N and the remaining are carbon atoms. wherein that aromatic ring system may be unsubstituted or mono-substituted with R^{Y4b};
Hetcyc^{X4} denotes a partially unsaturated monocyclic heterocycle with 5, 6, 7, 8 ring atoms wherein 1, 2, 3, 4 ring atom(s) is/are heteroatom(s) selected from N, O and/or S and the remaining ring atoms are carbon atoms, wherein that heterocycle may be unsubstituted or mono- or disubstituted with R^{X4b}, R^{X5b};
Hetcyc^{Y4} denotes a saturated monocyclic heterocycle with 3, 4, 5, 6, 7 ring atoms wherein 1 or 2 ring atom(s) is/are heteroatom(s) selected from N and/or O and the remaining ring atoms are carbon atoms, wherein that heterocycle may be unsubstituted or mono-substituted with R^{Y4b};
R^{X1f}, R^{X2f}, R^{X1g}, R^{X2g} denote independently from each other Hal, R^{X9d}, —CN, —NO₂, —SO₂NH₂, —SO₂—R^{X9d}, —NH₂, —NHR^{X7d}, —NR^{X7d}R^{X8d}, —NH—C(═O)—R^{X9d}, —OH, —O—R^{X9d}, —C(═O)—NH₂
R^{X4b}, R^{X5b} denote independently from each other oxo (═O), R^{X9d};
R^{Y4b} denotes NH₂, straight-chain or branched —C₁₋₆-alkyl;
R^{X7d}, R^{X8d}, R^{X9d} denote independently from each other straight-chain or branched —C₁₋₆-alkyl;
Hal denotes F, Cl, Br, I.

21. Compound according to any one of claims 16 to 20, or derivatives, N-oxides, prodrugs, solvates, tautomers or stereoisomers thereof as well as the physiologically acceptable salts of each of the foregoing, including mixtures thereof in all ratios,
wherein
R⁴ denotes pyridinyl, pyrazinyl, pyrimidinyl, methylpyridinyl, 4-methylpyridin-3-yl, methoxypyridinyl, 2-methoxy-pyridin-4-yl, 4-methoxy-pyridin-3-yl, 6-methoxy-pyridin-3-yl, aminopyridinyl, 2-amino-pyridin-4-yl, 6-aminopyridin-3-yl, methylaminopyridinyl, 6-methylaminopyridin-3-yl, methylpiperazinylpyridinyl, 4-(1-methylpiperazin-4-yl)pyridin-3-yl, methylpyrazolylpyridinyl, 4-(1-methyl-1H-pyrazol-4-yl)pyridin-3-yl, 5-(1-methyl-1H-pyrazolyl)pyridinyl, methylimidazolyl, 1-methyl-1H-imidazol-4-yl, 1-methyl-1H-imidazol-5-yl, methyltriazolyl, phenyl, 3-methoxyphenyl, 4-methoxyphenyl, 3-(SO₂NH₂)-phenyl (3-aminosulfonylphenyl), methyl-dihydropyridinonyl, 1-methyl-1,2-dihydropyridin-2-on-5-yl;
R⁵ denotes methyl, —CH₂—C(═O)—N(CH₃)₂, hydroxycyclohex-4-yl, aminocyclohex-4-yl, CH₃—C(═O)—NH-cyclohex-4-yl, acetylazetidinyl, 1-acetylazetidin-3-yl, piperidinyl, methylpiperidinyl, acetylpiperidinyl, N-cyanomethylpiperidinyl, N—(CH₃CH₂C(═O)-)piperidinyl, N—((CH₃)₂CH—C(═O)-)piperidinyl, 1-(2-methoxy-ethan-1-onyl)-piperdin-4-yl (1-(CH₃O—CH₂—C(═O)-)piperidin-4-yl), 1-(butan-1-on-1-yl)piperidin-4-yl, 1-(propan-2-on-1-yl)piperidin-4-yl (1-(CH₃—C(═O)—CH₂-)piperidin-4-yl, 1-(HOOC—CH₂-)piperidin-4-yl, 1-(CH₃—NH—C(═O)-)piperidin-4-yl, 1-((CH₃)₂N—C(═O)-)piperidin-4-yl, 1-(NH₂—C(═O)—CH₂)piperidin-4-yl, 1-(CH₃—NH—C(═O)—CH₂)piperidin-4-yl, 1-((CH₃)₂N—C(═O)—CH₂)piperidin-4-yl, 1-((CH₃CH₂)₂N—C(═O)—CH₂)piperidin-4-yl, 1-cyclopropanecarbonylpiperidin-4-yl, 1-(NH₂—CH₂—C(═O)-)piperidin-4-yl, 1-(CH₃—CH(—NH₂)—C(═O)-)piperidin-4-yl, 1-methanesulfonylpiperidin-4-yl, dihydropyridinonyl, 1-(NH₂—CH₂CH₂—C(═O)-)piperidin-4-yl, 1,2-dihydropyridin-2-on-5-yl (6-hydroxypyridin-3-yl), 1,2-dihydropyridin-2-on-4-yl (2-hydroxypyridin-4-yl), oxanyl, imidazolyl, methylimidazolyl, 1-methyl-1H-imidazol-5-yl, pyrazolyl, methylpyrazolyl, 1-methyl-1H-pyrazol-5-yl, triazolyl, methyltriazolyl, 1-methyl-1H-1,2,3-triazol-5-yl, tetrazolyl, methyltetrazolyl, 1-methyl-1H-1,2,3,4-tetrazol-5-yl or pyridinyl.

22. Compound according to any one of claims 16 to 21, or derivatives, N-oxides, prodrugs, solvates, tautomers or stereoisomers thereof as well as the physiologically acceptable salts of each of the foregoing, including mixtures thereof in all ratios,
wherein
R⁴ denotes pyridin-3-yl, pyridin-4-yl, pyrazin-2-yl, 4-methylpyridin-3-yl, 2-methoxy-pyridin-4-yl, 6-methoxy-pyridin-3-yl, 2-aminopyridin-4-yl, 6-aminopyridin-3-yl, 4-(1-methylpiperazin-4-yl)pyridin-3-yl, 4-(1-methyl-1H-pyrazol-4-yl)pyridin-3-yl, 5-(1-methyl-1H-pyrazol-4-yl)pyridin-3-yl, 1-methyl-1H-imidazol-5-yl, 1-methyl-1H-1,2,3-triazol-5-yl;
R⁵ denotes methyl, aminocyclohex-4-yl, CH₃—C(═O)—NH—cyclohex-4-yl, piperidin-4-yl, 1-acetylpiperidin-3-yl, N-acetylpiperidin-4-yl, N-methylpiperidin-4-yl, 1-cyanomethylpiperidin-4-yl, 1-(CH₃CH₂C(═O)-)piperidin-4-yl (1-(ethylcarbonyl)piperidin-4-yl), 1-((CH₃)₂CH—C(═O)-)piperidin-4-yl, 1-(2-methoxy-ethan-1-onyl)-piperdin-4-yl (1-(CH₃O—CH₂—C(═O)-)piperidin-4-yl), 1-(butan-1-on-1-yl)piperidin-4-yl, 1-(propan-2-on-1-yl)piperidin-4-yl, 1-cyclopropanecarbonylpiperidin-4-yl, 1-(CH₃—NH—C(═O)-)piperidin-4-yl, 1-((CH₃)₂N—C(═O)-)piperidin-4-yl, 1-(NH₂—C(═O)—CH₂)piperidin-4-yl, 1-(CH₃—NH—C(═O)—CH₂)piperidin-4-yl, 1-((CH₃)₂N—C(═O)—CH₂)piperidin-4-yl, 1,2-dihydropyridin-2-on-5-yl (6-hydroxypyridin-3-yl), 1,2-dihydropyridin-2-on-4-yl, 1-methyl-1H-imidazol-5-yl, 1-methyl-1H-1,2,3-triazol-5-yl, oxan-4-yl or pyridin-3-yl.

23. Compound according to any one of claims 1 to 10, or derivatives, N-oxides, prodrugs, solvates, tautomers or stereoisomers thereof as well as the physiologically acceptable salts of each of the foregoing, including mixtures thereof in all ratios,
wherein
R⁵, R⁶ both denote independently from each other Ar^{X}, Hetar^{X}, Hetcyc^{X}, LA^{X} or
R⁵ and R⁶ form together with the carbon atom to which they are attached to a saturated ring system D which ring system D is mono- or bicyclic and has 3, 4, 5, 6, 7, 8, 9, 10 ring atoms and may contain no hetero ring atom or 1, 2, 3 hetero ring atom(s) independently from each other selected from N, O and/or S that ring system D may be unsubstituted or mono-, di- or trisubstituted with independently from each other R^{D1}, R^{D2}, R^{D3};
R^{D1}, R^{D2}, R^{D3} are as defined in claim 1.

24. Compound according to claim 23, or derivatives, N-oxides, prodrugs, solvates, tautomers or stereoisomers thereof as well as the physiologically acceptable salts of each of the foregoing, including mixtures thereof in all ratios,
wherein
R⁵ denotes LA^{X5};
R⁶ denotes LA^{X6};
or
R⁵ and R⁶ form together with the carbon atom to which they are attached to a saturated ring system D which ring system D is mono- or bicyclic and has 3, 4, 5, 6, 7, ring atoms and may contain no hetero ring atom or 1 hetero ring atom selected from N, O and/or S that ring system D may be unsubstituted or mono-substituted with straight-chain or branched C₁₋₆-alkyl;
LA^{X5}, LA^{X6} denote independently from each other straight-chain or branched —C₁₋₆-alkyl.

25. Compound according to any of claims 23 or 24, or derivatives, N-oxides, prodrugs, solvates, tautomers or stereoisomers thereof as well as the physiologically acceptable salts of each of the foregoing, including mixtures thereof in all ratios,
wherein
R⁵ and R⁶ both have the same meaning.

26. Compound according to any of claims 23 to 25, or derivatives, N-oxides, prodrugs, solvates, tautomers or stereoisomers thereof as well as the physiologically acceptable salts of each of the foregoing, including mixtures thereof in all ratios,
wherein
R⁵ and R⁶ both denote methyl;
or
R⁵ and R⁶ form together with the carbon atom to which they are attached to a saturated ring system D which ring system D is selected from 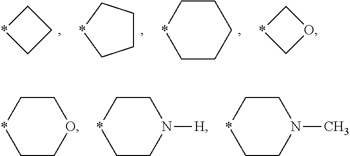
wherein the “*” denotes the carbon atom to which R⁵ and R⁶ are attached to.

27. Compound according to any one of claims 1 to 10, or derivatives, N-oxides, prodrugs, solvates, tautomers or stereoisomers thereof as well as the physiologically acceptable salts of each of the foregoing, including mixtures thereof in all ratios,
wherein
R⁴ and R⁵ form together with the carbon atom to which they are attached to a saturated or partially unsaturated ring system A which ring system A is mono- or bicyclic and has 3, 4, 5, 6, 7, 8, 9, 10 ring atoms and may contain no hetero ring atom or 1, 2, 3 hetero ring atom(s) independently from each other selected from N, O and/or S that ring system A may be unsubstituted or mono-, di- or trisubstituted with independently from each other R^{A1}, R^{A2}, R^{A3}
R^{A1}, R^{A2}, R^{A3} are as defined in claim 1.

28. Compound according to claim 27, or derivatives, N-oxides, prodrugs, solvates, tautomers or stereoisomers thereof as well as the physiologically acceptable salts of each of the foregoing, including mixtures thereof in all ratios,
wherein
R⁴ and R⁵ form together with the carbon atom to which they are attached to a saturated or partially unsaturated ring system A which ring system A is mono- or bicyclic and has 4, 5, 6, 7, 9, 10 ring atoms and may contain no hetero ring atom or 1, 2, 3 hetero ring atom(s) independently from each other selected from N, O and/or S that ring system A may be unsubstituted or mono-, di-substituted with independently from each other R^{A1a}, R^{A2a};
R^{A1a}, R^{A2a} denote independently from each other LA^{XA}, —C(═O)—R^{X9A}, oxo (═O), —NH—C(═O)—R^{X9A}, —SO₂—R^{X9A}, phenyl, pyridinyl, methylpyridinyl, pyrimidinyl, hydroxypyrimidinyl, methylpyrimidinyl, pyrazinyl, benzodiazolyl or form together with one carbon atom of ring system A to which they both are attached to a saturated ring system E which ring system E is mono-cyclic and has 3, 4, 5, 6, 7 ring atoms and may contain no hetero atom or 1 hetero atom selected from N and O, that ring system E may be unsubstituted or mono- or di-substituted with independently from each other R^{E1a}, R^{E1b};
LA^{XA}, R^{E1a}, R^{E1b} denote independently from each other straight-chain or branched —C₁₋₆-alkyl;
R^{X9A} denotes straight-chain or branched —C₁₋₆-alkyl, which may be unsubstituted or monosubstituted with —NH₂, a saturated monocyclic carbocycle with 3, 4, 5, 6, 7 carbon atoms, phenyl or pyridinyl.

29. Compound according to any of claims 27 or 28, or derivatives, N-oxides, prodrugs, solvates, tautomers or stereoisomers thereof as well as the physiologically acceptable salts of each of the foregoing, including mixtures thereof in all ratios,
wherein
R⁴ and R⁵ form together with the carbon atom to which they are attached to
(i) a saturated or partially unsaturated monocyclic ring system A with 4, 5, 6 or 7 ring atoms which may contain no hetero ring atom or 1 hetero ring atom selected from N and O that ring system A may be unsubstituted or mono-, di-substituted with independently from each other R^{A1a}, R^{A2a}, or
(ii) a saturated or partially unsaturated bicyclic ring system A with 9 or 10 ring atoms which may contain no hetero ring atom or 1 hetero ring atom selected from N and O that ring system A may be unsubstituted or mono-, di-substituted with independently from each other R^{A1a}, R^{A2a};
R^{A1a}, R^{A2a} denote independently from each other methyl, —C(═O)-methyl, —C(═O)-ethyl, —C(═O)—CH(CH₃)₂, —C(═O)-(cyclo-C₃H₅), —C(═O)-phenyl, —C(═O)-pyridinyl, —C(═O)—CH₂NH₂, oxo (═O), —NH—C(═O)-methyl, —SO₂-methyl, phenyl, pyridin-2-yl, pyridin-3-yl, 3-methylpyridin-2-yl, pyrimidin-2-yl, pyrimidin-4-yl, pyrimidin-5-yl, 2-hydroxypyrimidin-4-yl, 2-methylpyrimidin-4-yl, pyrazin-2-yl, 1H-1,3-benzodiazol-2-yl or form together with one carbon atom of ring system A to which they both are attached to a saturated ring 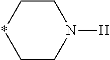 [SMILES: *1CCNCC1]
wherein the “*” denotes the carbon atom to which R^{A1a} and R^{A2a} are attached to.

30. Compound according to any of claims 27 to 29, or derivatives, N-oxides, prodrugs, solvates, tautomers or stereoisomers thereof as well as the physiologically acceptable salts of each of the foregoing, including mixtures thereof in all ratios,
wherein
R⁴ and R⁵ form together with the carbon atom to which they are attached to oxanyl, dimethyloxanyl, tetrahydronaphthalenyl, tetrahydroquinolinyl, N-acetyltetrahydroquinolinyl, dihydrobenzopyranyl, azetidinyl, N-acetylazetidinyl, pyrrolidinyl, N-methylpyrrolidinyl, N-phenylpyrrolidinyl, N-acetylpyrrolidinyl, N-ethylcarbonylpyrrolidinyl, N—((CH₃)₂—CH—C(═O)-)pyrrolidinyl, N-cyclopropanecarbonylpyrrolidinyl, N-benzoylpyrrolidinyl, N-(pyridinylcarbonyl)pyrrolidinyl, N-(aminomethylcarbonyl)pyrrolidinyl, N-methanesulfonylpyrrolidinyl, N-(pyridinyl)pyrrolidinyl, N-(methylpyridinyl)pyrrolidinyl, N-(pyrimidinyl)pyrrolidinyl, N-(hydroxypyrimidinyl)pyrrolidinyl, N-(methylpyrimidinyl)pyrrolidinyl, N-(pyranzinyl)pyrrolidinyl, piperidinyl, N-acetylpiperidinyl, N-(pyrimidinyl)piperidinyl, N-(benzodiazolyl)pyrrolidinyl, azepanyl, N-acetylazepanyl, N-cyclopropanecarbonylazepanyl, 7-azaspiro[3.5]nonan-1-yl, (CH₃—C(═O)—NH—)cyclohexyl, cyclohexanonyl, piperidinonyl, 2H,3H,4H-pyrano[3,2-b]pyridin-4-yl, 5,6,7,8-tetrahydroquinoxalin-5-yl.

31. Compound according to any of claims 27 to 29, or derivatives, N-oxides, prodrugs, solvates, tautomers or stereoisomers thereof as well as the physiologically acceptable salts of each of the foregoing, including mixtures thereof in all ratios,
wherein
R⁴ and R⁵ form together with the carbon atom to which they are attached to oxan-4-yl, 2,3-dimethyloxan-4-yl, 1,2,3,4-tetrahydronaphthalen-1-yl, 5,6,7,8-tetrahydroquinolin-5-yl, 5,6,7,8-tetrahydroquinolin-8-yl, N-acetyl-1,2,3,4-tetrahydroquinolin-4-yl, 3,4-dihydro-2H-1-benzopyran-4-yl, cyclohexan-4-onyl, 2H,3H,4H-pyrano[3,2-b]pyridin-4-yl, 5,6,7,8-tetrahydroquinoxalin-5-yl, 1-acetylazetidin-3-yl, pyrrolidin-3-yl, 1-methylpyrroldin-3-yl, 1-phenylpyrrolidin-3-yl, 1-acetylpyrrolidin-3-yl, 1-(ethylcarbonyl)pyrrolidin-3-yl, 1-((CH₃)₂—CH—C(═O)-)pyrrolidin-3-yl, 1-cyclopropanecarbonylpyrrolidin-3-yl, 1-benzoylpyrrolidin-3-yl, 1-(pyridin-2-ylcarbonyl)pyrrolidin-3-yl, 1-(aminomethylcarbonyl)pyrrolidin-3-yl, 1-methanesulfonylpyrrolidin-3-yl, 1-(pyridin-2-yl)pyrrolidin-3-yl, 1-(pyridin-3-yl)pyrrolidin-3-yl, 1-(3-methylpyridin-2-yl)pyrrolidin-3-yl, 1-(pyrimidin-2-yl)pyrroldin-3-yl, 1-(pyrimidin-4-yl)pyrroldin-3-yl, 1-(pyrimidin-5-yl)pyrroldin-3-yl, 1-(2-hydroxypyrimidin-4-yl)pyrrolidin-3-yl, 1-(2-methylpyrimidin-4-yl)pyrrolidin-3-yl, 1-(pyranzin-2-yl)pyrrolidin-3-yl, 1-(1H-1,3-benzodiazol-2-yl)pyrrolidin-3-yl, 1-acetylpiperidin-3-yl, 1-acetylpiperidin-4-yl, 1-(pyrimidin-2-yl)piperidin-4-yl, 1-acetylazepan-4-yl, 1-(cyclopropanecarbonyl)azepan-4-yl, 1-(CH₃—C(═O)—NH—)cyclohex-4-yl.

32. Compound according to any of claims 1 to 31, or the N-oxides and/or physiologically acceptable salts thereof selected from the group consisting of:
8-(1-methyl-1H-indol-6-yl)-N-[(1R)-1,2,3,4-tetrahydronaphthalen-1-yl]quinoxalin-6-amine
8-(1-methyl-1H-indol-6-yl)-N-[1-(pyridin-3-yl)ethyl]quinoxalin-6-amine
8-(1-methyl-1H-indol-6-yl)-N-[2-(pyridin-3-yl)ethyl]quinoxalin-6-amine
8-(1-methyl-1H-indol-6-yl)-N-[1-(pyridin-4-yl)ethyl]quinoxalin-6-amine
8-(1-methyl-1H-indol-6-yl)-N-[1-(pyridin-2-yl)ethyl]quinoxalin-6-amine
N-[(1 S)-1-(3-methoxyphenyl)ethyl]-8-(1-methyl-1H-indol-6-yl)quinoxalin-6-amine
2-methoxy-4-(7-{[(1R)-1,2,3,4-tetrahydronaphthalen-1-yl]amino}quinoxalin-5-yl)benzonitrile
8-(1-methyl-1H-1,3-benzodiazol-6-yl)-N-[(1R)-1,2,3,4-tetrahydronaphthalen-1-yl]quinoxalin-6-amine
8-chloro-N-[(1R)-1,2,3,4-tetrahydronaphthalen-1-yl]quinoxalin-6-amine
8-(1-methyl-1H-indol-6-yl)-N-(pyridin-3-ylmethyl)quinoxalin-6-amine
N-[(1R)-1-(3-methoxyphenyl)ethyl]-8-(1-methyl-1H-indol-6-yl)quinoxalin-6-amine
8-(4-amino-3-methoxyphenyl)-N-[(1R)-1,2,3,4-tetrahydronaphthalen-1-yl]quinoxalin-6-amine
8-(5-amino-6-methylpyridin-3-yl)-N-[(1R)-1,2,3,4-tetrahydronaphthalen-1-yl]quinoxalin-6-amine
N-(3,4-dihydro-2H-1-benzopyran-4-yl)-8-(1-methyl-1H-indol-6-yl)quinoxalin-6-amine
N-[1-(4-methoxyphenyl)ethyl]-8-(1-methyl-1H-indol-6-yl)quinoxalin-6-amine
8-(1-methyl-1H-indol-6-yl)-N-(5,6,7,8-tetrahydroisoquinolin-8-yl)quinoxalin-6-amine
8-(2,3-dihydro-1,4-benzodioxin-6-yl)-N-[(1R)-1,2,3,4-tetrahydronaphthalen-1-yl]quinoxalin-6-amine
2-methoxy-4-(7-{[(1R)-1,2,3,4-tetrahydronaphthalen-1-yl]amino}quinoxalin-5-yl)benzamide
8-(1-methyl-1H-indol-6-yl)-N-(5,6,7,8-tetrahydroquinolin-5-yl)quinoxalin-6-amine
8-(1,3-dimethyl-1H-pyrazol-4-yl)-N-[(1R)-1,2,3,4-tetrahydronaphthalen-1-yl]quinoxalin-6-amine
2-{[8-(1-methyl-1H-indol-6-yl)quinoxalin-6-yl]amino}-1-(pyrrolidin-1-yl)-propan-1-one
N-(2,2-dimethyloxan-4-yl)-8-(1-methyl-1H-indol-6-yl)quinoxalin-6-amine
8-(1-methyl-1H-indol-6-yl)-N-(oxan-3-ylmethyl)quinoxalin-6-amine
8-(3-amino-4-methoxyphenyl)-N-[(1R)-1,2,3,4-tetrahydronaphthalen-1-yl]quinoxalin-6-amine
8-(4-methoxy-3-nitrophenyl)-N-[(1R)-1,2,3,4-tetrahydronaphthalen-1-yl]-quinoxalin-6-amine
8-chloro-N-[1-(pyridin-3-yl)ethyl]quinoxalin-6-amine
8-(1-methyl-1H-indol-6-yl)-N-(1,3-thiazol-4-ylmethyl)quinoxalin-6-amine
3-(1-{[8-(1-methyl-1H-indol-6-yl)quinoxalin-6-yl]amino}ethyl)benzene-1-sulfonamide
1-methyl-6-(7-{[(1R)-1,2,3,4-tetrahydronaphthalen-1-yl]amino}quinoxalin-5-yl)-1H,6H,7H-pyrrolo[2,3-c]pyridin-7-one
N-(furan-2-ylmethyl)-8-(1-methyl-1H-indol-6-yl)quinoxalin-6-amine
1-(4-{[8-(1-methyl-1H-indol-6-yl)quinoxalin-6-yl]amino}-1,2,3,4-tetrahydroquinolin-1-yl)ethan-1-one
N-benzyl-8-(1-methyl-1H-indol-6-yl)quinoxalin-6-amine
2-methyl-8-(1-methyl-1H-indol-6-yl)-N-[1-(pyridin-3-yl)ethyl]quinoxalin-6-amine
3-methyl-8-(1-methyl-1H-indol-6-yl)-N-[1-(pyridin-3-yl)ethyl]quinoxalin-6-amine
8-(1-methyl-1H-indol-6-yl)-N-[(1R)-1-(pyridin-3-yl)ethyl]quinoxalin-6-amine
8-(1-methyl-1H-indol-6-yl)-N-[(1 S)-1-(pyridin-3-yl)ethyl]quinoxalin-6-amine
8-(1-methyl-1H-indol-6-yl)-N-[1-(pyrazin-2-yl)ethyl]quinoxalin-6-amine
8-(1-methyl-1H-indol-6-yl)quinoxalin-6-ol
8-(1-methyl-1H-indol-6-yl)-N-(piperidin-3-yl)quinoxalin-6-amine
8-(1-methyl-1H-indol-6-yl)quinoxalin-6-amine
8-(1-methyl-1H-indol-6-yl)-N-[1-(pyrimidin-5-yl)ethyl]quinoxalin-6-amine
8-(1H-indazol-6-yl)-N-[1-(pyridin-3-yl)ethyl]quinoxalin-6-amine
5-(1-methyl-1H-indol-6-yl)-7-(pyridin-3-ylmethoxy)quinoxaline
8-{1-methyl-1H-pyrrolo[3,2-b]pyridin-6-yl}-N-[1-(pyridin-3-yl)ethyl]quinoxalin-6-amine
8-(1H-indol-6-yl)-N-[1-(pyridin-3-yl)ethyl]quinoxalin-6-amine
8-(1-methyl-1H-indol-6-yl)-6-{[1-(pyridin-3-yl)ethyl]amino}quinoxalin-2-ol
5-(1-methyl-1H-indol-6-yl)-7-{[1-(pyridin-3-yl)ethyl]amino}quinoxalin-2-ol
N-[bis(pyridin-3-yl)methyl]-8-(1-methyl-1H-indol-6-yl)quinoxalin-6-amine
N-[bis(pyridin-3-yl)methyl]-8-chloroquinoxalin-6-amine
8-{1-methyl-1H-pyrrolo[2,3-b]pyridin-6-yl}-N-[1-(pyridin-3-yl)ethyl]quinoxalin-6-amine
2,2,2-trifluoro-N-[8-(1-methyl-1H-indol-6-yl)quinoxalin-6-yl]-N-(piperidin-4-yl)acetamide
8-[1-(2-methoxyethyl)-1H-indol-6-yl]-N-[1-(pyridin-3-yl)ethyl]quinoxalin-6-amine
N-[(4-methanesulfonylphenyl)methyl]-8-(1-methyl-1H-indol-6-yl)quinoxalin-6-amine
8-(1-methyl-1H-indol-6-yl)-N-(pyridazin-3-ylmethyl)quinoxalin-6-amine
N-[(3-methanesulfonylphenyl)methyl]-8-(1-methyl-1H-indol-6-yl)quinoxalin-6-amine
N-[(2-methanesulfonylphenyl)methyl]-8-(1-methyl-1H-indol-6-yl)quinoxalin-6-amine
8-(1-methyl-1H-indol-6-yl)-N-(piperidin-2-ylmethyl)quinoxalin-6-amine
8-(1-methyl-1H-indol-6-yl)-N-(piperidin-3-ylmethyl)quinoxalin-6-amine
5-(7-{[1-(pyridin-3-yl)ethyl]amino}quinoxalin-5-yl)-2,3-dihydro-1H-isoindol-1-one
8-(1-methyl-1H-indol-6-yl)-N-(morpholin-2-ylmethyl)quinoxalin-6-amine
8-(1-methyl-1H-indol-6-yl)-N-(1H-pyrazol-4-ylmethyl)quinoxalin-6-amine
8-(1,3-benzothiazol-6-yl)-N-[1-(pyridin-3-yl)ethyl]quinoxalin-6-amine
3-{[8-(1-methyl-1H-indol-6-yl)quinoxalin-6-yl]amino}-3-(pyridin-3-yl)prop-2-enoic acid
8-[3-(3-aminoazetidin-1-yl)phenyl]-N-[1-(pyridin-3-yl)ethyl]quinoxalin-6-amine
1-[6-(7-{[1-(pyridin-3-yl)ethyl]amino}quinoxalin-5-yl)-2,3-dihydro-1H-indol-1-yl]ethan-1-one
8-{octahydrocyclopenta[c]pyrrol-2-yl}-N-[1-(pyridin-3-yl)ethyl]quinoxalin-6-amine
8-(1-methyl-1H-indol-6-yl)-N-(oxan-4-yl)quinoxalin-6-amine
3-{[8-(1-methyl-1H-indol-6-yl)quinoxalin-6-yl]amino}-3-(pyridin-3-yl)propanoic acid
6-(7-{[1-(pyridin-3-yl)ethyl]amino}quinoxalin-5-yl)-4H-chromen-4-one
8-(1-methyl-1H-indol-6-yl)-N-{[5-(1-methyl-1H-pyrazol-4-yl)pyridin-3-yl]methyl}quinoxalin-6-amine
4-({[8-(1-methyl-1H-indol-6-yl)quinoxalin-6-yl]amino}methyl)benzonitrile
3-({[8-(1-methyl-1H-indol-6-yl)quinoxalin-6-yl]amino}methyl)benzonitrile
N-{[5-(1H-imidazol-1-yl)pyridin-3-yl]methyl}-8-(1-methyl-1H-indol-6-yl)quinoxalin-6-amine
N-{[5-(2-aminopyrimidin-5-yl)pyridin-3-yl]methyl}-8-(1-methyl-1H-indol-6-yl)quinoxalin-6-amine
8-(1-methyl-1H-indol-6-yl)-N-[(4-nitrophenyl)methyl]quinoxalin-6-amine
N-[(4-aminophenyl)methyl]-8-(1-methyl-1H-indol-6-yl)quinoxalin-6-amine
N-[1-(6-methoxypyridin-3-yl)ethyl]-8-(1-methyl-1H-indol-6-yl)quinoxalin-6-amine
8-(1-methyl-1H-indol-6-yl)-N-[(3-nitrophenyl)methyl]quinoxalin-6-amine
N-[(3-aminophenyl)methyl]-8-(1-methyl-1H-indol-6-yl)quinoxalin-6-amine
4-{[8-(1-methyl-1H-indol-6-yl)quinoxalin-6-yl]amino}cyclohexan-1-one
5-{[8-(1-methyl-1H-indol-6-yl)quinoxalin-6-yl]amino}piperidin-2-one
8-(1-methyl-1H-indol-6-yl)-N-[2-(pyridin-3-yl)propan-2-yl]quinoxalin-6-amine
8-(1-methyl-1H-indol-5-yl)-N-[1-(pyridin-3-yl)ethyl]quinoxalin-6-amine
3-({[8-(1-methyl-1H-indol-6-yl)quinoxalin-6-yl]amino}methyl)benzamide
8-(1-methyl-1H-indol-6-yl)-N-{[3-(1H-1,2,3,4-tetrazol-5-yl)phenyl]methyl}quinoxalin-6-amine
N-[(2-methoxypyridin-3-yl)methyl]-8-(1-methyl-1H-indol-6-yl)quinoxalin-6-amine
3-({[8-(1-methyl-1H-indol-6-yl)quinoxalin-6-yl]amino}methyl)-1,2-dihydropyridin-2-one
4-({[8-(1-methyl-1H-indol-6-yl)quinoxalin-6-yl]amino}methyl)benzamide
8-(1-methyl-1H-indol-6-yl)-N-{[4-(1H-1,2,3,4-tetrazol-5-yl)phenyl]methyl}quinoxalin-6-amine
N-methyl-8-(1-methyl-1H-indol-6-yl)-N-(pyridin-3-ylmethyl)quinoxalin-6-amine
8-(1-methyl-1H-indol-6-yl)-N-[(8S)-5,6,7,8-tetrahydroisoquinolin-8-yl]quinoxalin-6-amine
8-(1-methyl-1H-indol-6-yl)-N-[(8R)-5,6,7,8-tetrahydroisoquinolin-8-yl]quinoxalin-6-amine
8-(1-methyl-1H-indol-4-yl)-N-[1-(pyridin-3-yl)ethyl]quinoxalin-6-amine
4-{[8-(1-methyl-1H-indol-6-yl)quinoxalin-6-yl]amino}piperidin-2-one
8-(1-methyl-1H-indol-6-yl)-N-{[5-(1H-pyrazol-4-yl)pyridin-3-yl]methyl}quinoxalin-6-amine
N-[(5-bromopyridin-3-yl)methyl]-8-(1-methyl-1H-indol-6-yl)quinoxalin-6-amine
8-(1-methyl-1H-indol-6-yl)-N-(piperid in-4-yl)quinoxalin-6-amine
8-(3-methyl-1-benzofuran-5-yl)-N-{1-[5-(1-methyl-1H-pyrazol-4-yl)pyridin-3-yl]ethyl}quinoxalin-6-amine
8-(1-methyl-1H-indol-6-yl)-N-{[5-(pyrimidin-5-yl)pyridin-3-yl]methyl}quinoxalin-6-amine
N-[(5-aminopyridin-3-yl)methyl]-8-(1-methyl-1H-indol-6-yl)quinoxalin-6-amine
8-(1-methyl-1H-indol-6-yl)-N-{[5-(1H-pyrazol-5-yl)pyridin-3-yl]methyl}quinoxalin-6-amine
8-(3-methyl-1-benzofuran-5-yl)-N-(oxan-4-yl)quinoxalin-6-amine
1-(4-{[8-(1-methyl-1H-indol-6-yl)quinoxalin-6-yl]amino}piperidin-1-yl)ethan-1-one
N-{7-azaspiro[3.5]nonan-1-yl}-8-(1-methyl-1H-indol-6-yl)quinoxalin-6-amine
8-(1-methyl-1H-indol-6-yl)-N-[piperidin-4-yl(pyridin-3-yl)methyl]quinoxalin-6-amine
8-(1-methyl-1H-indol-6-yl)-N-{[5-(morpholin-4-yl)pyridin-3-yl]methyl}quinoxalin-6-amine
8-(3-methyl-1-benzofuran-5-yl)-N-(morpholin-2-ylmethyl)quinoxalin-6-amine
8-(1-methyl-1H-indol-6-yl)-N-[(4-methylpyridin-3-yl)methyl]quinoxalin-6-amine
N-[(4-fluoropyridin-3-yl)methyl]-8-(1-methyl-1H-indol-6-yl)quinoxalin-6-amine
5-({[8-(1-methyl-1H-indol-6-yl)quinoxalin-6-yl]amino}methyl)pyridin-3-ol
3-(7-{[1-(pyridin-3-yl)ethyl]amino}quinoxalin-5-yl)benzene-1-sulfonamide
8-(1-methyl-1H-indol-6-yl)-N-(5,6,7,8-tetrahydroquinoxalin-5-yl)quinoxalin-6-amine
8-(3-methyl-1-benzofuran-5-yl)-N-[(1 S)-1-[3-(1-methyl-1H-pyrazol-4-yl)phenyl]ethyl]quinoxalin-6-amine
N-[1-(pyridin-3-yl)ethyl]-8-(quinolin-6-yl)quinoxalin-6-amine
8-(1-methyl-1H-indol-6-yl)-N-[oxan-4-yl(pyridin-3-yl)methyl]quinoxalin-6-amine
8-(1-methyl-1H-indol-6-yl)-N-[(1-methylpiperidin-2-yl)methyl]quinoxalin-6-amine
5-({[8-(1-methyl-1H-indol-6-yl)quinoxalin-6-yl]amino}methyl)-1,2-dihydropyridin-2-one
N-[1-(pyridin-3-yl)ethyl]-8-(quinolin-7-yl)quinoxalin-6-amine
8-(1-methyl-1H-indol-6-yl)-N-{2H,3H,4H-pyrano[3,2-b]pyridin-4-yl}quinoxalin-6-amine
1-[2-({[8-(1-methyl-1H-indol-5-yl)quinoxalin-6-yl]amino}methyl)piperidin-1-yl]ethan-1-one
N-[(2-aminopyrimidin-5-yl)methyl]-8-(1-methyl-1H-indol-6-yl)quinoxalin-6-amine
8-(3-methyl-1-benzofuran-5-yl)-N-{[5-(1-methyl-1H-pyrazol-4-yl)pyridin-3-yl]methyl}quinoxalin-6-amine
1-[4-({[8-(1-methyl-1H-indol-6-yl)quinoxalin-6-yl]amino}(pyridin-3-yl)methyl)piperidin-1-yl]ethan-1-one
N-[(2-chloropyrimidin-5-yl)methyl]-8-(1-methyl-1H-indol-6-yl)quinoxalin-6-amine
8-(1-methyl-1H-indol-6-yl)-N-[(4-methylmorpholin-2-yl)methyl]quinoxalin-6-amine
8-(1-methyl-1H-indol-6-yl)-N-{[4-(pyrimidin-5-yl)pyridin-3-yl]methyl}quinoxalin-6-amine
8-(1-methyl-1H-indol-6-yl)-N-{[4-(4-methylpiperazin-1-yl)pyridin-3-yl]methyl}quinoxalin-6-amine
N-{imidazo[1,2-a]pyridin-6-ylmethyl}-8-(1-methyl-1H-indol-6-yl)quinoxalin-6-amine
8-(1-methyl-1H-indol-6-yl)-N-{[4-(1-methyl-1H-pyrazol-4-yl)pyridin-3-yl]methyl}quinoxalin-6-amine
1-[2-({[8-(1-methyl-1H-indol-6-yl)quinoxalin-6-yl]amino}methyl)morpholin-4-yl]ethan-1-one
8-(1-methyl-1H-indol-6-yl)-N-(morpholin-3-ylmethyl)quinoxalin-6-amine
1-methyl-4-{[8-(1-methyl-1H-indol-6-yl)quinoxalin-6-yl]amino}piperidin-2-one
1-methyl-5-{[8-(1-methyl-1H-indol-6-yl)quinoxalin-6-yl]amino}piperidin-2-one
N-[(1-methyl-1H-imidazol-5-yl)methyl]-8-(1-methyl-1H-indol-6-yl)quinoxalin-6-amine
N-[(4-bromopyridin-2-yl)methyl]-8-(1-methyl-1H-indol-6-yl)quinoxalin-6-amine
8-(1-methyl-1H-indol-6-yl)-N-{[4-(1-methyl-1H-pyrazol-4-yl)pyridin-2-yl]methyl}quinoxalin-6-amine
N-[(2-bromopyridin-4-yl)methyl]-8-(1-methyl-1H-indol-6-yl)quinoxalin-6-amine
8-(1-methyl-1H-indol-6-yl)-N-{[2-(1-methyl-1H-pyrazol-4-yl)pyridin-4-yl]methyl}quinoxalin-6-amine
N-[(1-methyl-1H-1,2,3-triazol-5-yl)(pyridin-3-yl)methyl]-8-(1-methyl-1H-indol-6-yl)quinoxalin-6-amine
8-(1-methyl-1H-indol-6-yl)-N-[(1-methylpiperidin-4-yl)(pyridin-3-yl)methyl]quinoxalin-6-amine
N-[(4-benzylmorpholin-3-yl)methyl]-8-(1-methyl-1H-indol-6-yl)quinoxalin-6-amine
8-(1-methyl-1H-indol-6-yl)-N-{[4-(pyrimidin-5-yl)morpholin-2-yl]methyl}quinoxalin-6-amine
8-(1-methyl-1H-indol-6-yl)-N-[piperidin-4-yl(pyridin-4-yl)methyl]-quinoxalin-6-amine
8-(1-methyl-1H-indol-6-yl)-N-[piperidin-4-yl(pyridazin-3-yl)methyl]-quinoxalin-6-amine
N-[(4-aminopyridin-3-yl)methyl]-8-(1-methyl-1H-indol-6-yl)quinoxalin-6-amine
N-[(4-methoxypyridin-3-yl)methyl]-8-(1-methyl-1H-indol-6-yl)quinoxalin-6-amine
1-{4-[3-({[8-(1-methyl-1H-indol-6-yl)quinoxalin-6-yl]amino}methyl)-pyridin-4-yl]piperazin-1-yl}ethan-1-one
1-[4-({[8-(3-methyl-1-benzofuran-5-yl)quinoxalin-6-yl]amino}(pyridin-3-yl)methyl)piperidin-1-yl]ethan-1-one
N-[(1-methyl-1H-imidazol-4-yl)(piperidin-4-yl)methyl]-8-(1-methyl-1H-indol-6-yl)quinoxalin-6-amine
N-[(6-methoxypyridin-3-yl)(pyridin-3-yl)methyl]-8-(1-methyl-1H-indol-6-yl)quinoxalin-6-amine
2-methyl-1-[4-({[8-(1-methyl-1H-indol-6-yl)quinoxalin-6-yl]amino}-(pyridin-3-yl)methyl)piperidin-1-yl]propan-1-one
1-[4-({[8-(1-methyl-1H-indol-6-yl)quinoxalin-6-yl]amino}(pyridin-3-yl)methyl)piperidin-1-yl]propan-1-one
2-[4-({[8-(1-methyl-1H-indol-6-yl)quinoxalin-6-yl]amino}(pyridin-3-yl)methyl)piperidin-1-yl]acetonitrile
N-[(2-methoxypyridin-4-yl)(pyridin-3-yl)methyl]-8-(1-methyl-1H-indol-6-yl)quinoxalin-6-amine
8-(1-methyl-1H-indol-6-yl)-N-{1-[4-(1-methyl-1H-pyrazol-4-yl)pyridin-3-yl]ethyl}quinoxalin-6-amine
8-(1-methyl-1H-indol-6-yl)-N-{1-[4-(4-methylpiperazin-1-yl)pyridin-3-yl]ethyl}quinoxalin-6-amine
N-[(1-methyl-1H-1,2,3-triazol-5-yl)methyl]-8-(1-methyl-1H-indol-6-yl)quinoxalin-6-amine
5-({[8-(1-methyl-1H-indol-6-yl)quinoxalin-6-yl]amino}(pyridin-3-yl)methyl)-1,2-dihydropyridin-2-one
N-[(1-cyclopropanecarbonylpiperidin-4-yl)(pyridin-3-yl)methyl]-8-(1-methyl-1H-indol-6-yl)quinoxalin-6-amine
8-(1-methyl-1H-indol-6-yl)-N-[pyridin-3-yl(pyridin-4-yl)methyl]quinoxalin-6-amine
1-[4-({[8-(1-methyl-1H-indol-6-yl)quinoxalin-6-yl]amino}(pyridin-3-yl)methyl)piperidin-1-yl]propan-2-one
1-[4-({[8-(1-methyl-1H-indol-6-yl)quinoxalin-6-yl]amino}(pyridin-3-yl)methyl)piperidin-1-yl]butan-1-one
1-[3-((S){[8-(1-methyl-1H-indol-6-yl)quinoxalin-6-yl]amino}(pyridin-3-yl)methyl)piperidin-1-yl]ethan-1-one
1-[3-((R){[8-(1-methyl-1H-indol-6-yl)quinoxalin-6-yl]amino}(pyridin-3-yl)methyl)piperidin-1-yl]ethan-1-one
3-({[8-(1-methyl-1H-indol-6-yl)quinoxalin-6-yl]amino}methyl)pyridine-4-carbonitrile
2-[4-({[8-(1-methyl-1H-indol-6-yl)quinoxalin-6-yl]amino}(pyridin-3-yl)methyl)piperidin-1-yl]acetic acid
2-[4-({[8-(1-methyl-1H-indol-6-yl)quinoxalin-6-yl]amino}(pyridin-3-yl)methyl)piperidin-1-yl]acetamide
1-{4-[(6-methoxypyridin-3-yl)({[8-(1-methyl-1H-indol-6-yl)quinoxalin-6-yl]amino})methyl]piperidin-1-yl}ethan-1-one
2-methoxy-1-[4-({[8-(1-methyl-1H-indol-6-yl)quinoxalin-6-yl]amino}(pyridin-3-yl)methyl)piperidin-1-yl]ethan-1-one
8-(1-methyl-1H-indol-6-yl)-N-[pyridin-3-yl(pyrimidin-5-yl)methyl]-quinoxalin-6-amine
N-[(6-methoxypyridin-3-yl)(pyridin-3-yl)methyl]-8-(3-methyl-1-benzothiophen-5-yl)quinoxalin-6-amine
8-(1-methyl-1H-indol-6-yl)-N-(1,3-oxazol-5-ylmethyl)quinoxalin-6-amine
8-(1-methyl-1H-indol-6-yl)-N-(1,2-thiazol-4-ylmethyl)quinoxalin-6-amine
8-(1-methyl-1H-indol-6-yl)-N-(1,2-oxazol-4-ylmethyl)quinoxalin-6-amine
8-(1-methyl-1H-indol-6-yl)-N-(1,3-thiazol-5-ylmethyl)quinoxalin-6-amine
5-({[8-(3-methyl-1-benzothiophen-5-yl)quinoxalin-6-yl]amino}(pyridin-3-yl)methyl)-1,2-dihydropyridin-2-one
2-amino-1-[4-({[8-(1-methyl-1H-indol-6-yl)quinoxalin-6-yl]amino}(pyridin-3-yl)methyl)piperidin-1-yl]ethan-1-one
N-[(1-methyl-1H-imidazol-5-yl)(pyridin-3-yl)methyl]-8-(1-methyl-1H-indol-6-yl)quinoxalin-6-amine
1-{4-[(1-methyl-1H-1,2,3-triazol-5-yl)({[8-(1-methyl-1H-indol-6-yl)-quinoxalin-6-yl]amino})methyl]piperidin-1-yl}ethan-1-one
4-({[8-(1-methyl-1H-indol-6-yl)quinoxalin-6-yl]amino}(pyridin-3-yl)methyl)-1,2-dihydropyridin-2-one
8-(3-methyl-1-benzothiophen-5-yl)-N-[piperidin-4-yl(pyridin-3-yl)methyl]quinoxalin-6-amine
N-[4-({[8-(1-methyl-1H-indol-6-yl)quinoxalin-6-yl]amino}(pyridin-3-yl)methyl)cyclohexyl]acetamide
1-[4-({[8-(3-methyl-1-benzothiophen-5-yl)quinoxalin-6-yl]amino}(pyridin-3-yl)methyl)piperidin-1-yl]ethan-1-one
N—[(S)-(6-methoxypyridin-3-yl)(pyridin-3-yl)methyl]-8-(1-methyl-1H-indol-6-yl)quinoxalin-6-amine
N—[(R)-(6-methoxypyridin-3-yl)(pyridin-3-yl)methyl]-8-(1-methyl-1H-indol-6=yl)quinoxalin-6-amine
N,N-dimethyl-3-{[8-(1-methyl-1H-indol-6-yl)quinoxalin-6-yl]amino}-3-(pyridin-3-yl)propanamide
2-amino-1-[4-({[8-(1-methyl-1H-indol-6-yl)quinoxalin-6-yl]amino}(pyridin-3-yl)methyl)piperidin-1-yl]propan-1-one
N-methyl-2-[4-({[8-(1-methyl-1H-indol-6-yl)quinoxalin-6-yl]amino}-(pyridin-3-yl)methyl)piperidin-1-yl]acetamide
N,N-dimethyl-2-[4-({[8-(1-methyl-1H-indol-6-yl)quinoxalin-6-yl]amino}-(pyridin-3-yl)methyl)piperidin-1-yl]acetamide
N,N-diethyl-2-[4-({[8-(1-methyl-1H-indol-6-yl)quinoxalin-6-yl]amino}-(pyridin-3-yl)methyl)piperidin-1-yl]acetamide
3-amino-1-[4-({[8-(1-methyl-1H-indol-6-yl)quinoxalin-6-yl]amino}(pyridin-3-yl)methyl)piperidin-1-yl]propan-1-one
8-(1-methyl-1H-indol-6-yl)-N-[(4-methyl-4H-1,2,4-triazol-3-yl)methyl]-quinoxalin-6-amine
N-[(3-methyl-1,2-thiazol-5-yl)methyl]-8-(1-methyl-1H-indol-6-yl)-quinoxalin-6-amine
8-(1-methyl-1H-indol-6-yl)-N-[(1,2-thiazol-5-yl)methyl]quinoxalin-6-amine
N-[(5-methyl-1,3,4-oxadiazol-2-yl)methyl]-8-(1-methyl-1H-indol-6-yl)quinoxalin-6-amine
N-[(5-methyl-1H-1,2,4-triazol-3-yl)methyl]-8-(1-methyl-1H-indol-6-yl)quinoxalin-6-amine
N-(1H-imidazol-4-ylmethyl)-8-(1-methyl-1H-indol-6-yl)quinoxalin-6-amine
N-[(1,2-dimethyl-1H-imidazol-5-yl)methyl]-8-(1-methyl-1H-indol-6-yl)-quinoxalin-6-amine
8-(1-methyl-1H-indol-6-yl)-N-(4H-1,2,4-triazol-3-ylmethyl)quinoxalin-6-amine
1-[4-({[8-(1-methyl-1H-indol-6-yl)quinoxalin-6-yl]amino}(4-methylpyridin-3-yl)methyl)piperidin-1-yl]ethan-1-one
N-[(2-aminopyridin-4-yl)(pyridin-3-yl)methyl]-8-(1-methyl-1H-indol-6-yl)quinoxalin-6-amine
1-[3-({[8-(1-methyl-1H-indol-6-yl)quinoxalin-6-yl]amino}(pyridin-3-yl)methyl)azetidin-1-yl]ethan-1-one
N-[(1-methyl-1H-imidazol-4-yl)(pyridin-3-yl)methyl]-8-(1-methyl-1H-indol-6-yl)quinoxalin-6-amine
1-[4-({[8-(2-amino-1,3-benzothiazol-5-yl)quinoxalin-6-yl]amino}(6-methoxypyridin-3-yl)methyl)piperidin-1-yl]ethan-1-one
1-[4-({[8-(4-bromophenyl)quinoxalin-6-yl]amino}(pyridin-3-yl)methyl)-piperidin-1-yl]ethan-1=one
1-[4-({[8-(2-amino-1,3-benzothiazol-5-yl)quinoxalin-6-yl]amino}(pyridin-3-yl)methyl)piperidin-1-yl]ethan-1-one
5-[(1-acetylpiperidin-4-yl)({[8-(1-methyl-1H-indol-6-yl)quinoxalin-6-yl]amino})methyl]-1-methyl-1,2-dihydropyridin-2-one
8-(2-amino-1,3-benzothiazol-5-yl)-N-[(6-methoxypyridin-3-yl)(pyridin-3-yl)methyl]quinoxalin-6-amine
N-[(6-aminopyridin-3-yl)(pyridin-3-yl)methyl]-8-(1-methyl-1H-indol-6-yl)quinoxalin-6-amine
N-[(6-methoxypyridin-3-yl)(pyridin-3-yl)methyl]-N-methyl-8-(1-methyl-1H-indol-6-yl)quinoxalin-6-amine
N-methyl-4-({[8-(1-methyl-1H-indol-6-yl)quinoxalin-6-yl]amino}(pyridin-3-yl)methyl)piperidine-1-carboxamide
N-[(6-methoxypyridin-3-yl)(1-methyl-1H-1,2,3-triazol-5-yl)methyl]-8-(1-methyl-1H-indol-6-yl)quinoxalin-6-amine
N,N-dimethyl-4-({[8-(1-methyl-1H-indol-6-yl)quinoxalin-6-yl]amino}-(pyridin-3-yl)methyl)piperidine-1-carboxamide
3-({[8-(1-methyl-1H-indol-5-yl)quinoxalin-6-yl]amino}methyl)benzonitrile
3-({[8-(1-methyl-1H-indol-5-yl)quinoxalin-6-yl]amino}methyl)benzamide
1-(4-{[8-(1-ethyl-1H-indol-6-yl)quinoxalin-6-yl]amino}piperidin-1-yl)ethan-1-one
1-(4-{[8-(1-methyl-1H-indol-5-yl)quinoxalin-6-yl]amino}piperidin-1-yl)ethan-1-one
1-(4-{[8-(1-ethyl-1H-indol-5-yl)quinoxalin-6-yl]amino}piperidin-1-yl)ethan-1-one
1-[4-({8-[3-(dimethylamino)phenyl]quinoxalin-6-yl}amino)piperidin-1-yl]ethan-1-one
N-[(2-chloropyrimidin-5-yl)methyl]-8-(1-methyl-1H-indol-5-yl)quinoxalin-6-amine
1-(4-{[8-(1-benzyl-1H-indol-5-yl)quinoxalin-6-yl]amino}piperidin-1-yl)ethan-1-one
1-(4-{[8-(1-benzyl-1H-indol-6-yl)quinoxalin-6-yl]amino}piperidin-1-yl)ethan-1-one
1-[4-({8-[1-(propan-2-yl)-1H-indol-6-yl]quinoxalin-6-yl}amino)piperidin-1-yl]ethan-1-one
1-(4-{[8-(1-methyl-1H-indazol-6-yl)quinoxalin-6-yl]amino}piperid in-1-yl)ethan-1-one
1-(3-{[8-(1-methyl-1H-indol-6-yl)quinoxalin-6-yl]amino}pyrrolidin-1-yl)ethan-1-one
1-(3-{[8-(1-methyl-1H-indol-6-yl)quinoxalin-6-yl]amino}azetidin-1-yl)ethan-1-one
1-(4-{[8-(1-methyl-1H-1,3-benzodiazol-6-yl)quinoxalin-6-yl]amino}-piperidin-1-yl)ethan-1-one
1-(4-{[8-(2-methyl-2H-indazol-5-yl)quinoxalin-6-yl]amino}piperidin-1-yl)ethan-1-one
N-[(2-aminopyrimidin-5-yl)methyl]-8-(1-methyl-1H-indol-5-yl)quinoxalin-6-amine
1-[(3R)-3-{[8-(1-methyl-1H-indol-6-yl)quinoxalin-6-yl]amino}piperidin-1-yl]ethan-1-one
1-(5-{7-[(1-acetylpiperidin-4-yl)amino]quinoxalin-5-yl}pyridin-2-yl)ethan-1-one
N-[(5-bromopyridin-3-yl)methyl]-8-(1-methyl-1H-indazol-6-yl)quinoxalin-6-amine
1-[(3S)-3-{[8-(1-methyl-1H-indol-6-yl)quinoxalin-6-yl]amino}pyrrolidin-1-yl]ethan-1-one
1-[(3S)-3-{[8-(1-methyl-1H-indol-6-yl)quinoxalin-6-yl]amino}piperidin-1-yl]ethan-1-one
1-[(3S)-3-{[8-(1-methyl-1H-indazol-6-yl)quinoxalin-6-yl]amino}piperidin-1-yl]ethan-1-one
1-(4-{[8-(1H-1,3-benzodiazol-6-yl)quinoxalin-6-yl]amino}piperidin-1-yl)ethan-1-one
1-[(3R)-3-{[8-(1-methyl-1H-indol-6-yl)quinoxalin-6-yl]amino}pyrrolidin-1-yl]ethan-1-one
8-(1-methyl-1H-indol-6-yl)-N-(pyrrolidin-3-yl)quinoxalin-6-amine
1-[(3S)-3-{[8-(1-methyl-1H-indazol-6-yl)quinoxalin-6-yl]amino}pyrrolidin-1-yl]ethan-1-one
1-(4-{[8-(1H-indol-6-yl)quinoxalin-6-yl]amino}piperidin-1-yl)ethan-1-one
1-(4-{[8-(1-methyl-1H-indol-2-yl)quinoxalin-6-yl]amino}piperidin-1-yl)ethan-1-one
3-{7-[(1-acetylpyrrolidin-3-yl)amino]quinoxalin-5-yl}benzamide
1-(4-{[8-(2-methoxypyridin-4-yl)quinoxalin-6-yl]amino}piperidin-1-yl)ethan-1-one
1-(3-{[8-(1-methyl-1H-indol-6-yl)quinoxalin-6-yl]amino}pyrrolidin-1-yl)propan-1-one
1-(3-{[8-(1-methyl-1H-indazol-6-yl)quinoxalin-6-yl]amino}azetidin-1-yl)ethan-1-one
1-[(3S)-3-{[8-(1-methyl-2,3-dihydro-1H-indol-6-yl)quinoxalin-6-yl]amino}pyrrolidin-1-yl]ethan-1-one
1-(3-{[8-(3-methyl-1-benzothiophen-5-yl)quinoxalin-6-yl]amino}pyrrolidin-1-yl)ethan-1-one
1-(4-{[8-(1-methyl-2,3-dihydro-1H-indol-6-yl)quinoxalin-6-yl]amino}-piperidin-1-yl)ethan-1-one
N-(1-benzoylpyrrolidin-3-yl)-8-(1-methyl-1H-indol-6-yl)quinoxalin-6-amine
N-(1-methanesulfonylpyrrolidin-3-yl)-8-(1-methyl-1H-indol-6-yl)quinoxalin-6-amine
2-methyl-1-(3-{[8-(1-methyl-1H-indol-6-yl)quinoxalin-6-yl]amino}pyrrolidin-1-yl)propan-1-one
6-[(1-acetylpyrrolidin-3-yl)amino]-8-(1-methyl-1H-indol-6-yl)quinoxaline-2-carbonitrile
N-(1-cyclopropanecarbonylpyrrolidin-3-yl)-8-(1-methyl-1H-indol-6-yl)quinoxalin-6-amine
1-(3-{[8-(naphthalen-2-yl)quinoxalin-6-yl]amino}pyrrolidin-1-yl)ethan-1-one
1-(3-{[8-(1-methyl-1,2,3,4-tetrahydroquinolin-7-yl)quinoxalin-6-yl]amino}pyrrolidin-1-yl)ethan-1-one
1-[(3S)-3-({8-[3-(dimethylamino)-4-methylphenyl]quinoxalin-6-yl}amino)pyrrolidin-1-yl]ethan-1-one
1-(4-{[8-(1-methyl-1H-indol-6-yl)quinoxalin-6-yl]amino}azepan-1-yl)ethan-1-one
N-(1-cyclopropanecarbonylazepan-4-yl)-8-(1-methyl-1H-indol-6-yl)quinoxalin-6-amine
1-[(3S)-3-({8-[4-methyl-3-(methylamino)phenyl]quinoxalin-6-yl}amino)-pyrrolidin-1-yl]ethan-1-one
1-[(3S)-3-{[8-(1H-1,3-benzodiazol-2-yl)quinoxalin-6-yl]amino}pyrrolidin-1-yl]ethan-1-one
1-(4-{[8-(1-methyl-1,2,3,4-tetrahydroquinolin-7-yl)quinoxalin-6-yl]amino}-piperidin-1-yl)ethan-1-one
8-(1-methyl-1H-indol-6-yl)-N-[1-(pyridin-3-yl)pyrrolidin-3-yl]quinoxalin-6-amine
8-(1-methyl-1H-indol-6-yl)-N-[(3S)-1-(pyrimidin-4-yl)pyrrolidin-3-yl]-quinoxalin-6-amine
8-(1-methyl-1H-indol-6-yl)-N-[(3S)-1-(pyrimidin-2-yl)pyrrolidin-3-yl]-quinoxalin-6-amine
1-[(3S)-3-{[8-(5-methyl-2,3,4,5-tetrahydro-1H-1,5-benzodiazepin-7-yl)quinoxalin-6-yl]amino}pyrrolidin-1-yl]ethan-1-one
1-[(3S)-3-{[8-(4-methyl-1,2,3,4-tetrahydroquinoxalin-6-yl)quinoxalin-6-yl]amino}pyrrolidin-1-yl]ethan-1-one
1-[(3S)-3-({8-[3-(dimethylamino)-4-methoxyphenyl]quinoxalin-6-yl}amino)pyrrolidin-1-yl]ethan-1-one
8-(1-methyl-1H-indol-6-yl)-N-[(3S)-1-(pyridin-2-yl)pyrrolidin-3-yl]-quinoxalin-6-amine
8-(1-methyl-1H-indol-6-yl)-N-[(3S)-1-(pyrimidin-5-yl)pyrrolidin-3-yl]-quinoxalin-6-amine
4-[(3S)-3-{[8-(1-methyl-1H-indol-6-yl)quinoxalin-6-yl]amino}pyrrolidin-1-yl]pyrimidin-2-ol
8-(1-methyl-1H-indol-6-yl)-N-(1-phenylpyrrolidin-3-yl)quinoxalin-6-amine
8-(1-methyl-1H-indol-6-yl)-N-[1-(pyrimidin-2-yl)piperidin-4-yl]quinoxalin-6-amine
8-(1-methyl-1H-indol-6-yl)-N-[(3S)-1-methylpyrrolidin-3-yl]quinoxalin-6-amine
2-amino-1-[(3S)-3-{[8-(1-methyl-1H-indol-6-yl)quinoxalin-6-yl]amino}-pyrrolidin-1-yl]ethan-1-one
8-(1-methyl-1H-indol-6-yl)-N-[(3S)-1-(3-methylpyridin-2-yl)pyrrolidin-3-yl]quinoxalin-6-amine
1-[(3S)-3-[(8-{3-[ethyl(methyl(methyl)amino]-4-methylphenyl}quinoxalin-6-yl)amino]pyrrolidin-1-yl]ethan-1-one
8-(3-methyl-1H-indol-5-yl)-N-[(3S)-1-(pyrimidin-4-yl)pyrrolidin-3-yl]quinoxalin-6-amine
8-(1,3-dimethyl-1H-indol-5-yl)-N-[(3S)-1-(pyrimidin-2-yl)pyrrolidin-3-yl]quinoxalin-6-amine
8-(1-methyl-1H-indol-6-yl)-N-[(3S)-1-(pyrazin-2-yl)pyrrolidin-3-yl]-quinoxalin-6-amine
8-(1-methyl-1H-indol-6-yl)-N-[(3S)-1-(2-methylpyrimidin-4-yl)pyrrolidin-3-yl]quinoxalin-6-amine
8-(1-methyl-1H-indol-6-yl)-N-[(3S)-1-(pyridine-2-carbonyl)pyrrolidin-3-yl]quinoxalin-6-amine
N-[(3S)-1-(1H-1,3-benzodiazol-2-yl)pyrrolidin-3-yl]-8-(1-methyl-1H-indol-6-yl)quinoxalin-6-amine
N-[(1,4-cis)-4-{[8-(1-methyl-1H-indol-6-yl)quinoxalin-6-yl]amino}cyclohexyl]acetamide
N-(4-methanesulfonylpyridin-2-yl)-8-(1-methyl-1H-indol-6-yl)-N-[(pyridin-3-yl)methyl]quinoxalin-6-amine
N-(4-methanesulfonylpyridin-3-yl)-8-(1-methyl-1H-indol-6-yl)-N-[(pyridin-3-yl)methyl]quinoxalin-6-amine
8-(1-methyl-1H-indol-6-yl)-N-(pyridin-3-yl)-N-[(pyridin-4-yl)methyl]quinoxalin-6-amine
N-(1-methyl-1H-1,2,3-triazol-5-yl)-8-(1-methyl-1H-indol-6-yl)-N-[(pyridin-3-yl)methyl]quinoxalin-6-amine
1-[3-({[8-(1-methyl-1H-indol-6-yl)quinoxalin-6-yl](pyridin-3-yl)amino}methyl)piperidin-1-yl]ethan-1-one
N-(5-methanesulfonylpyridin-3-yl)-8-(1-methyl-1H-indol-6-yl)-N-[(pyridin-3-yl)methyl]quinoxalin-6-amine
N-(2-methanesulfonylpyridin-4-yl)-8-(1-methyl-1H-indol-6-yl)-N-[(pyridin-3-yl)methyl]quinoxalin-6-amine
3-{[8-(1-methyl-1H-indol-6-yl)quinoxalin-6-yl][(pyridin-3-yl)methyl]amino}pyridine-4-carboxamide
8-(1-methyl-1H-indol-6-yl)-N-[(1-methyl-1H-pyrazol-5-yl)methyl]-quinoxalin-6-amine
N-[(6-methoxypyridin-3-yl)(1-methyl-1H-1,2,3-triazol-5-yl)methyl]-8-(3-methyl-1-benzothiophen-5-yl)quinoxalin-6-amine
8-(1-methyl-1H-indol-6-yl)-N-({8-methyl-8-azabicyclo[3.2.1]octan-3-yl}(pyridin-3-yl)methyl)quinoxalin-6-amine
8-(1-methyl-1H-indol-6-yl)-N-{[6-(methylamino)pyridin-3-yl](pyridin-3-yl)methyl}quinoxalin-6-amine
8-(1-methyl-1H-indol-6-yl)-N-[(1-methyl-1H-pyrazol-4-yl)methyl]quinoxalin-6-amine
N-[5-({[8-(1-methyl-1H-indol-6-yl)quinoxalin-6-yl]amino}(pyridin-3-yl)methyl)pyridin-2-yl]acetamide
N-[(4-aminocyclohexyl)(pyridin-3-yl)methyl]-8-(1-methyl-1H-indol-6-yl)quinoxalin-6-amine
N-[bis(6-methoxypyridin-3-yl)methyl]-8-(1-methyl-1H-indol-6-yl)quinoxalin-6-amine
1-{4-[(R)-{[8-(3-methyl-1-benzothiophen-5-yl)quinoxalin-6-yl]amino}-(pyridin-3-yl)methyl]piperidin-1-yl}ethan-1-one
1-{4-[(S)-{[8-(3-methyl-1-benzothiophen-5-yl)quinoxalin-6-yl]amino}-(pyridin-3-yl)methyl]piperidin-1-yl}ethan-1-one
N-[(2-methyl-1,3-oxazol-5-yl)methyl]-8-(1-methyl-1H-indol-6-yl)quinoxalin-6-amine
8-(3-methyl-1-benzothiophen-5-yl)-N-[(1-methyl-1H-imidazol-5-yl)-(pyridin-3-yl)methyl]quinoxalin-6-amine
N-[(6-methoxypyridin-3-yl)(1-methyl-1H-imidazol-5-yl)methyl]-8-(1-methyl-1H-indol-6-yl)quinoxalin-6-amine
N-[(6-methoxypyridin-3-yl)(1-methyl-1H-imidazol-5-yl)methyl]-8-(3-methyl-1-benzothiophen-5-yl)quinoxalin-6-amine
N-[(6-methoxypyridin-3-yl)(1-methyl-1H-1,2,3-triazol-5-yl)methyl]-8-(3-methyl-1-benzofuran-5-yl)quinoxalin-6-amine
N-[(6-methoxypyridin-3-yl)(1-methyl-1H-pyrazol-5-yl)methyl]-8-(1-methyl-1H-indol-6-yl)quinoxalin-6-amine
N-[(1-methanesulfonylpiperidin-4-yl)(pyridin-3-yl)methyl]-8-(1-methyl-1H-indol-6-yl)quinoxalin-6-amine
N-[(6-methoxypyridin-3-yl)(1,2-thiazol-5-yl)methyl]-8-(1-methyl-1H-indol-6-yl)quinoxalin-6-amine
8-(1-methyl-1H-indol-6-yl)-N-{[2-(methylamino)pyridin-4-yl](pyridin-3-yl) methyl}quinoxalin-6-amine
1-methyl-5-({[8-(1-methyl-1H-indol-6-yl)quinoxalin-6-yl]amino}-(pyridin-3-yl)methyl)-1,2-dihydropyridin-2-one
1-[4-(2-{[8-(1-methyl-1H-indol-6-yl)quinoxalin-6-yl]amino}-2-(pyridin-3-yl)ethyl)piperidin-1-yl]ethan-1-one
N-[(6-methoxypyridin-3-yl)(1,3-oxazol-5-yl)methyl]-8-(1-methyl-1H-indol-6-yl)quinoxalin-6-amine
8-(1-methyl-1H-indol-6-yl)-N-[2-(1-methylpyrrolidin-3-yl)-1-(pyridin-3-yl)-ethyl]quinoxalin-6-amine
4-({[8-(1-methyl-1H-indol-6-yl)quinoxalin-6-yl]amino}(pyridin-3-yl)methyl)cyclohexan-1-ol
N-[1,1-bis(pyridin-3-yl)ethyl]-8-(1-methyl-1H-indol-6-yl)quinoxalin-6-amine
N-[4-({[8-(3-methyl-1-benzothiophen-5-yl)quinoxalin-6-yl]amino}(pyridin-3-yl)methyl)pyridin-2-yl]acetamide
N-[(6-methoxypyridin-3-yl)methyl]-8-(1-methyl-1H-indol-6-yl)quinoxalin-6-amine
N-[(6-methoxypyridin-3-yl)(1-methyl-1H-1,2,3,4-tetrazol-5-yl)methyl]-8-(1-methyl-1H-indol-6-yl)quinoxalin-6-amine
N-[(6-methoxypyridin-3-yl)methyl]-8-(1-methyl-1H-indol-6-yl)quinoxalin-6-amine
8-(1-methyl-1H-indol-6-yl)-N-(pyridazin-4-ylmethyl)quinoxalin-6-amine
N—[(R)-(6-methoxypyridin-3-yl)(1-methyl-1H-1,2,3-triazol-5-yl)methyl]-8-(3-methyl-1-benzothiophen-5-yl)quinoxalin-6-amine
N—[(S)-(6-methoxypyridin-3-yl)(1-methyl-1H-1,2,3-triazol-5-yl)methyl]-8-(3-methyl-1-benzothiophen-5-yl)quinoxalin-6-amine
N—[(R)-(6-methoxypyridin-3-yl)(1-methyl-1H-1,2,3-triazol-5-yl)methyl]-8-(1-methyl-1H-indol-6-yl)quinoxalin-6-amine
N—[(S)-(6-methoxypyridin-3-yl)(1-methyl-1H-1,2,3-triazol-5-yl)methyl]-8-(1-methyl-1H-indol-6-yl)quinoxalin-6-amine
N-[(1R,4r)-4-[(R)-{[8-(1-methyl-1H-indol-6-yl)quinoxalin-6-yl]amino}-(pyridin-3-yl)methyl]cyclohexyl]acetamide
N-[(1S,4r)-4-[(S)-{[8-(1-methyl-1H-indol-6-yl)quinoxalin-6-yl]amino}-(pyridin-3-yl)methyl]cyclohexyl]acetamide
[8-(1-Methyl-1H-indol-6-yl)-quinoxalin-6-yl]-(1-oxy-pyridin-3-ylmethyl)-amine

33. A pharmaceutical composition comprising at least one compound of formula (I) as defined in any one of claims 1 to 32, or its derivatives, N-oxides, prodrugs, solvates, tautomers or stereoisomers thereof as well as the physiologically acceptable salts of each of the foregoing, including mixtures thereof in all ratios, as active ingredient, together with a pharmaceutically acceptable carrier.

34. The pharmaceutical composition according to claim 33 that further comprises a second active ingredient or its derivatives, N-oxides, prodrugs, solvates, tautomers or stereoisomers thereof as well as the physiologically acceptable salts of each of the foregoing, including mixtures thereof in all ratios, wherein that second active ingredient is other than a compound of formula (I) as defined in any one of claims 1 to 32.

35. Medicament comprising at least one compound of formula (I) as defined in any one of claims 1 to 32, or its derivatives, N-oxides, prodrugs, solvates, tautomers or stereoisomers thereof as well as the physiologically acceptable salts of each of the foregoing, including mixtures thereof in all ratios.

36. A compound of formula (I) as defined in any one of claims 1 to 32, or its derivatives, N-oxides, prodrugs, solvates, tautomers or stereoisomers thereof as well as the physiologically acceptable salts of each of the foregoing, including mixtures thereof in all ratios, for use in the prevention and/or treatment of medical conditions that are affected by inhibiting 6-phosphofructo-2-kinase/fructose-2,6-bisphosphatase (PFKFB), in particular PFKFB3.

37. A compound of formula (I) as defined in any one of claims 1 to 32, or its derivatives, N-oxides, prodrugs, solvates, tautomers or stereoisomers thereof as well as the physiologically acceptable salts of each of the foregoing, including mixtures thereof in all ratios, for use in the prevention and/or treatment of cancer, in particular adipose cancer, anogenital cancer, bladder cancer, breast cancer, central nervous system cancer, cervical cancer, colon cancer, connective tissue cancer, glioblastoma, glioma, kidney cancer, leukemia, lung cancer, lymphoid cancer, ovarian cancer, pancreatic cancer, prostate cancer, retinal cancer, skin cancer, stomach cancer, uterine cancer.

38. Set (kit) comprising separate packs of
a) an effective amount of a compound of formula (I) as defined in any one of claims 1 to 32, or its derivatives, N-oxides, prodrugs, solvates, tautomers or stereoisomers thereof as well as the physiologically acceptable salts of each of the foregoing, including mixtures thereof in all ratios; and
b) an effective amount of a further active ingredient that further active ingredient not being a compound of formula (I) as defined in any one of claims 1 to 32.

39. Process for manufacturing a compound according to any one of claims 1 to 32, or derivatives, N-oxides, prodrugs, solvates, tautomers or stereoisomers thereof as well as the physiologically acceptable salts of each of the foregoing, the process being characterized in that
(a) a compound of formula (II) 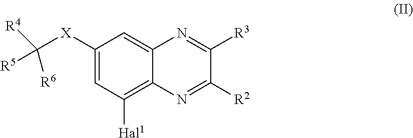
wherein
Hal¹ denotes Cl, Br or I;
R², R³, R⁴, R⁵, R⁶, X have the same meaning as defined in claims 1 to 31 for compounds of formula (I);
is reacted under C-C coupling reaction conditions which conditions may utilize one or more suitable C-C coupling reaction reagents including catalysts
with a compound R¹—RG^{a}
wherein
R¹ have the same meaning as defined in claims 1 to 31 for compounds of formula (I);
RG^{a} denotes a chemical moiety being reactive under the particular C-C coupling reaction conditions utilized;
or
(b) a compound of formula (III) 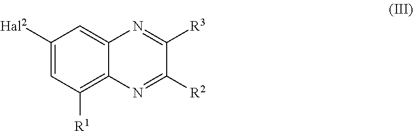
wherein
Hal² denotes Cl, Br or I;
R¹, R², R³ have the same meaning as defined in claims 1 to 31 for compounds of formula (I);
is reacted under C—N coupling reaction conditions which conditions may utilize one or more suitable C—N coupling reaction reagents including catalysts
with a compound R⁴R⁵R⁶C—NR⁷
wherein
X denotes N—R⁷;
R⁴, R⁵, R⁶, R⁷ have the same meaning as defined in claims 1 to 31 for compounds of formula (I);
RG^{b} denotes a chemical moiety being reactive under the particular C—N coupling reaction conditions utilized;
or
(c) a compound of formula (III) 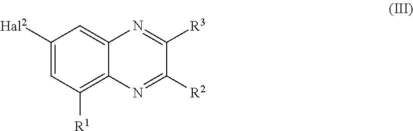
wherein
Hal² denotes Cl, Br or I;
R¹, R², R³ have the same meaning as defined in claims 1 to 31 for compounds of formula (I);
is reacted under C—O coupling reaction conditions which conditions may utilize one or more suitable C—O coupling reaction reagents including catalysts
with a compound R⁴R⁵R⁶C—OH
wherein
X denotes O;
R⁴, R⁵, R⁶ have the same meaning as defined in claims 1 to 31 for compounds of formula (I).

40. Compound of formula (II) or (III) 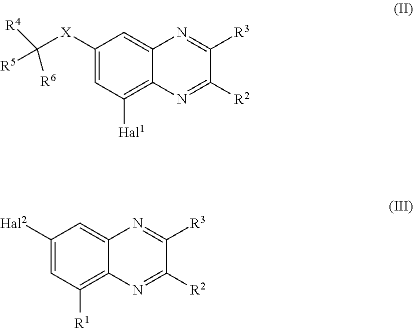
or salts thereof,
wherein
Hal¹ and Hal² denote independently from each other Cl, Br or I;
R¹, R², R³, R⁴, R⁵, R⁶, X have the same meaning as defined in claims 1 to 31 for compounds of formula (I)
with the proviso that
7-chloro-5-[2-fluoro-4-methyl-5-(2,2,2-trifluoroethanesulfinyl)phenyl]-quinoxaline and
7-chloro-5-{2-fluoro-4-methyl-5-[(2,2,2-trifluoroethyl)sulfanyl]-phenyl}quinoxaline are excluded.
